(19) United States
(12) Patent Application Publication
(10) Pub. No.: US 2026/0190613 A1
(43) Pub. Date: Jul. 2, 2026

(54) Organic Compound And Organic Light-Emitting Device Comprising Same

(71) Applicant: P&H TECH Co., Ltd, Gyeonggi-do (KR)

(72) Inventors: Seung Jae LEE, Gyeonggi-do (KR); Gwan-hee PARK, Gyeonggi-do (KR); In-Ho LEE, Gyeonggi-do (KR)

(73) Assignee: P&H TECH Co., Ltd, Gyeonggi-do (KR)

(21) Appl. No.: 18/998,118

(22) PCT Filed: Jul. 27, 2023
(86) PCT No.: PCT/KR2023/010894

§ 371 Date: Nov. 11, 2025

(30) Foreign Application Priority Data
Jul. 27, 2022 (KR) 10-2022-0093011

(51) Int. Cl. H10K 50/18; C07B 59/00; C07D 307/91; H10K 85/60

(52) U.S. Cl. CPC H10K 50/181; C07B 59/002; C07D 307/91; H10K 85/622; H10K 85/626; H10K 85/633; H10K 85/636; H10K 85/6572; H10K 85/6574; H10K 85/6576; C07B 2200/05

(57) ABSTRACT

Disclosed are an organic compound employed as an organic layer material, such as an electron-blocking layer in an organic light-emitting device; and an organic light-emitting device comprising same. When the compound according to the present invention is employed for an electron-blocking layer in a device, it is possible to implement an organic light-emitting device having significantly improved low voltage driving characteristics and device characteristics such as long lifespan and luminous efficiency, and thus the present invention can be industrially useful for various lighting devices and display devices.

### CROSS-REFERENCE TO RELATED APPLICATIONS

This application claims priority to and the benefit of Korean Patent Application No. 10-2022-0093011 filed in the Korean Intellectual Property Office on Jul. 27, 2022, the entire contents of which are incorporated herein by reference.

### TECHNICAL FIELD

The present invention relates to an organic compound, and more particularly, to an organic compound that is employed to organic layers such as an electron blocking layer, etc. provided in an organic light emitting device and an organic light emitting device that employs the same, thus achieving greatly improved device characteristics such as low-voltage driving of the device, long lifespan, and excellent luminous efficiency.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

However, in order for such an organic light emitting device to exhibit the aforementioned characteristics, the materials constituting an organic layer in the device, such as hole injecting materials, hole transport materials, hole blocking materials, light emitting materials, electron transport materials, electron injecting materials, and electron blocking materials, are prerequisites for the support by stable and efficient materials. However, the development of a stable and efficient organic layer material for an organic light emitting device has not yet been sufficiently made.

Therefore, there is a continuous need for developing a new material capable of improving the luminescent characteristics and an organic layer structure in the device.

### SUMMARY OF THE INVENTION

Thus, the present invention has been made in an effort to provide a novel organic compound which may be employed in organic layers such as an electron blocking layer, etc. provided in an organic light-emitting device to implement excellent luminescent properties such as low-voltage driving of the device, long lifespan, and excellent luminous efficiency, and an organic light-emitting device including the same.

An aspect of the present invention provides an organic compound represented by [Formula I] below and an organic light-emitting device in which the organic compound is included in the device.

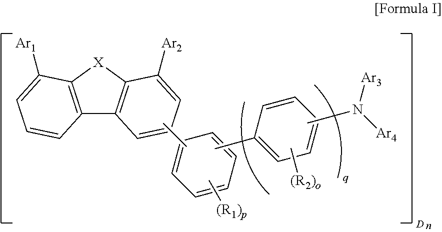

The characteristic structure of [Formula I] above and the definitions of compounds implemented thereby, X, R₁ to R₂, Ar₁ to Ar₄, o, p, q, D (deuterium), and n will be described below.

An organic light-emitting device employing the organic compound according to the present invention in an organic layer such as an electron blocking layer has significantly excellent device characteristics such as low-voltage driving, long lifespan characteristics, and luminous efficiency compared to conventional devices, and thus, can be usefully used in various lighting devices and display devices.

### DETAILED DESCRIPTION

Hereinafter, the present invention will be described in more detail.

The present invention relates to an organic compound represented by the following [Formula I], and when the organic compound is employed in various organic layers, preferably as an electron blocking layer, in an organic light-emitting device, it is possible to implement an organic light-emitting device having significantly improved device characteristics such as low-voltage driving, long lifespan, and luminous efficiency.

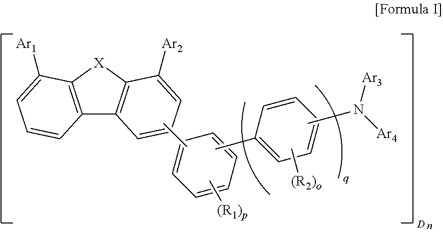

In [Formula I],
X is O or S.
Ar₁ and Ar₂ are each independently selected from hydrogen, a substituted or unsubstituted aryl group having 6 to 20 carbon atoms, and a substituted or unsubstituted heteroaryl group having 3 to 30 carbon atoms.

In this case, it is characterized in that the case where Ar₁ and Ar₂ are all hydrogen is excluded, that is, at least one of Ar₁ and Ar₂ is a substituted or unsubstituted aryl group/heteroaryl group.

Ar₃ and Ar₄ are each independently selected from a substituted or unsubstituted aryl group having 6 to 20 carbon atoms and a substituted or unsubstituted heteroaryl group having 3 to 30 carbon atoms.

R₁ and R₂ are the same as or different from each other, and are each independently hydrogen or deuterium or selected from a substituted or unsubstituted aryl group having 6 to 20 carbon atoms and a substituted or unsubstituted heteroaryl group having 3 to 30 carbon atoms.

p and o are each an integer from 0 to 4, and when p and o are 2 or higher, a plurality of R₁ and R₂ are each the same as or different from each other.

q is an integer from 0 to 2, and when q is 2, the structures in a plurality of ( ) are the same as or different from each other.

D is deuterium, n means the number of hydrogen atoms in [Formula I] above, which are replaced with deuterium atoms (D), and n is an integer from 0 to 60.

In [Formula I] above, not only the backbone structure but also R₁ to R₂ introduced therein may be each deuterium, or a compound in which R₁ to R₂ and Ar₁ to Ar₄ are each partially substituted with deuterium (D), that is, R₁ to R₂ and Ar₁ to Ar₄ each may include at least one deuterium as a substituent.

Therefore, according to an exemplary embodiment of the present invention, the deuterium (D) substitution rate in [Formula I] according to the present invention may be 10 to 90%.

As described above, the compound according to the present invention may be a compound containing at least one deuterium atom by replacing some hydrogen atoms in the [Formula I] structure with deuterium, and thus makes it possible to implement an organic light-emitting device having longevity by compensating for the short service life disadvantage of an organic light emitting device confirmed by a conventional moiety structure.

Meanwhile, in the definitions of R₁, R₂, and Ar₁ to Ar₄ above, the ‘substituted or unsubstituted’ means substitution of R₁, R₂, and Ar₁ to Ar₄ above with one or at least two substituents selected from the group consisting of deuterium, a cyano group, a halogen group, a hydroxy group, a nitro group, an alkyl group, an alkoxy group, a halogenated alkoxy group, a cycloalkyl group, a heterocycloalkyl group, an aryl group, a fluorenyl group, a heteroaryl group, and a silyl group, substitution with a substituent to which two or more of the substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

Furthermore, according to an exemplary embodiment of the present invention, the compound according to the present invention may include at least one deuterium atom in the structure of [Formula I].

Accordingly, R₁ and R₂ may each be deuterium, and Ar₁ to Ar₄ may each be a substituent substituted with at least one deuterium.

In an embodiment of the present invention, examples of the substituents will be described in detail below, but are not limited thereto.

In an embodiment of the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

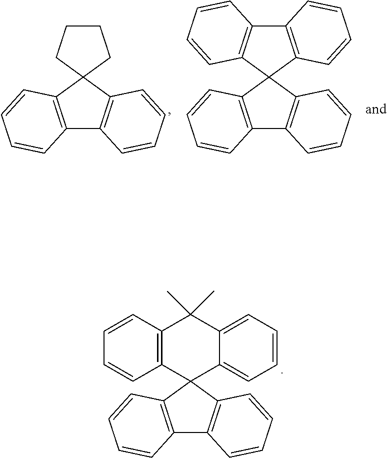

In an embodiment of the present invention, the fluorenyl groups include open structures in which one of the two cyclic organic compounds linked through one atom is cleaved, and examples thereof include

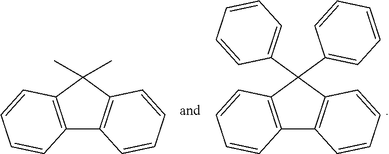

In addition, carbon atoms of the ring may be substituted with any one or more heteroatoms selected from among N, S and O, and examples thereof include

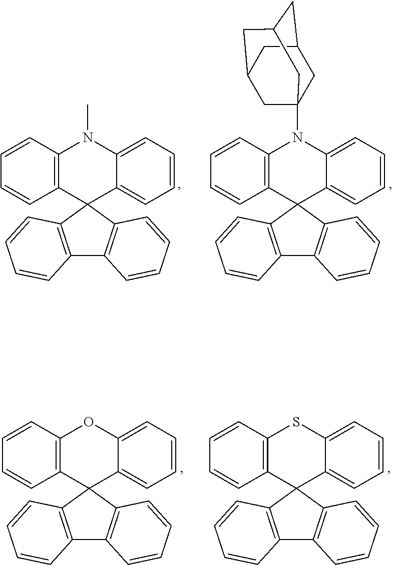

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (Cl), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

In an embodiment of the present invention, the amine group may be —NH₂, an alkylamine group, an arylamine group, an arylheteroarylamine group, etc., the arylamine group refers to amine substituted with an aryl group, the alkylamine group refers to amine substituted with an alkyl group, and the arylheteroarylamine group refers to amine substituted with aryl and heteroaryl groups. Examples of the arylamine group include a substituted or unsubstituted monoarylamine group, a substituted or unsubstituted diarylamine group, or a substituted or unsubstituted triarylamine group. The aryl group and the heteroaryl group in the arylamine group and the arylheteroarylamine group may be a monocyclic aryl group, a monocyclic heteroaryl group, a polycyclic aryl group, or a polycyclic heteroaryl group, and the arylamine group and the arylheteroarylamine group including two or more aryl groups and heteroaryl groups may include a monocyclic aryl group (heteroaryl group), a polycyclic aryl group (heteroaryl group), or both a monocyclic aryl group (heteroaryl group) and a polycyclic aryl group (heteroaryl group). In addition, the aryl group and the heteroaryl group in the arylamine group and the arylheteroarylamine group may be selected from examples of the above-mentioned aryl group and heteroaryl group.

Furthermore, various specific examples of the substituent according to the present invention can be clearly confirmed in the specific compounds described below.

The organic compound according to the present invention represented by [Formula I] above may be used as an organic layer of an organic light-emitting device due to its structural specificity as described above, and more specifically, may be used as a material for an electron blocking layer, and the like of the organic layer depending on the characteristics of various substituents to be introduced.

Preferred specific examples of the organic compound represented by Formula I according to the present invention include the following compounds but are not limited thereto.

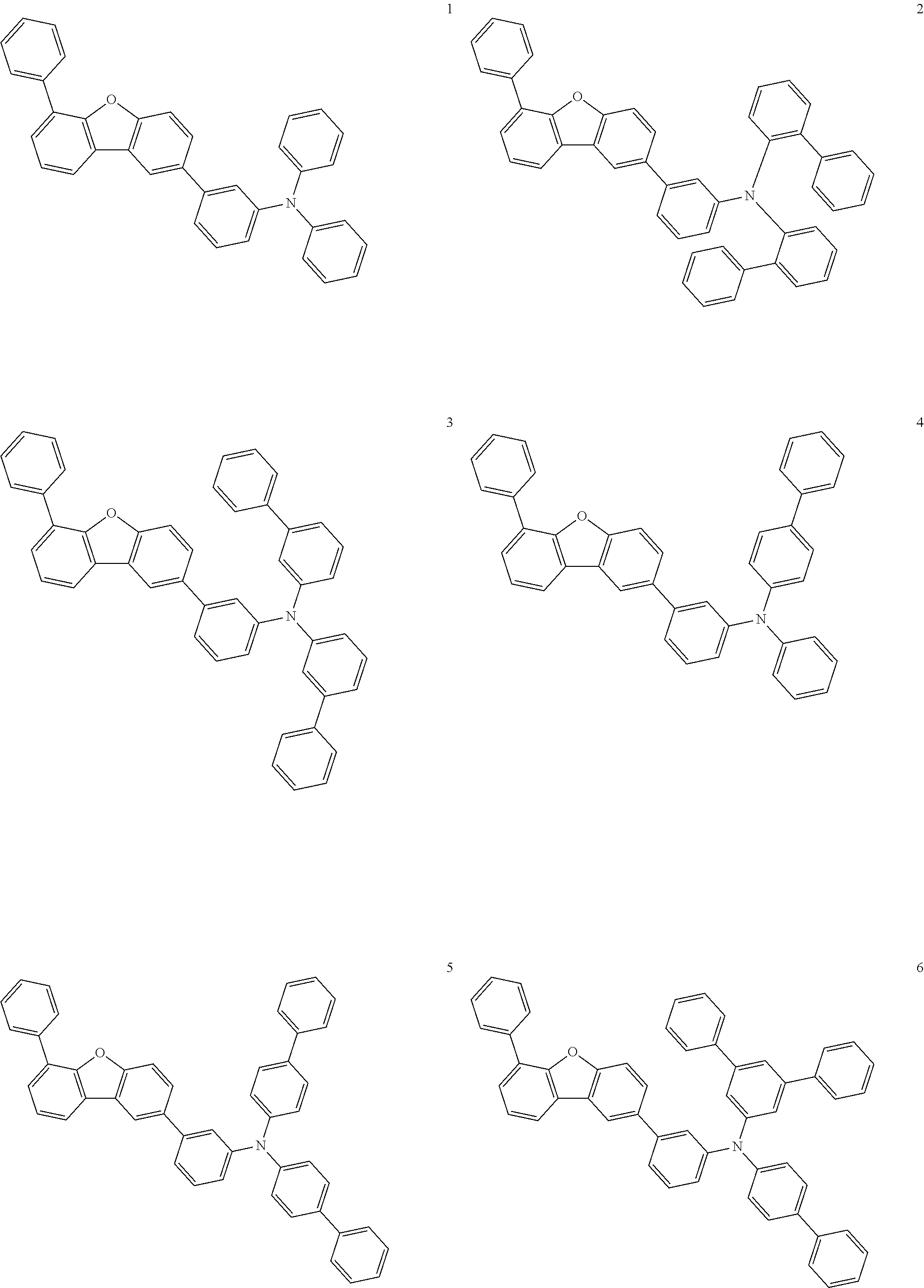 [SMILES: c1ccc(-c2cccc3c2oc2ccc(-c4cccc(N(c5ccccc5)c5ccccc5)c4)cc23)cc1 ; c1ccc(-c2ccccc2N(c2cccc(-c3ccc4oc5c(-c6ccccc6)cccc5c4c3)c2)c2ccccc2-c2ccccc2)cc1 ; c1ccc(-c2cccc(N(c3cccc(-c4ccccc4)c3)c3cccc(-c4ccc5oc6c(-c7ccccc7)cccc6c5c4)c3)c2)cc1 ; c1ccc(-c2ccc(N(c3ccccc3)c3cccc(-c4ccc5oc6c(-c7ccccc7)cccc6c5c4)c3)cc2)cc1 ; c1ccc(-c2ccc(N(c3ccc(-c4ccccc4)cc3)c3cccc(-c4ccc5oc6c(-c7ccccc7)cccc6c5c4)c3)cc2)cc1 ; c1ccc(-c2ccc(N(c3cccc(-c4ccc5oc6c(-c7ccccc7)cccc6c5c4)c3)c3cc(-c4ccccc4)cc(-c4ccccc4)c3)cc2)cc1]

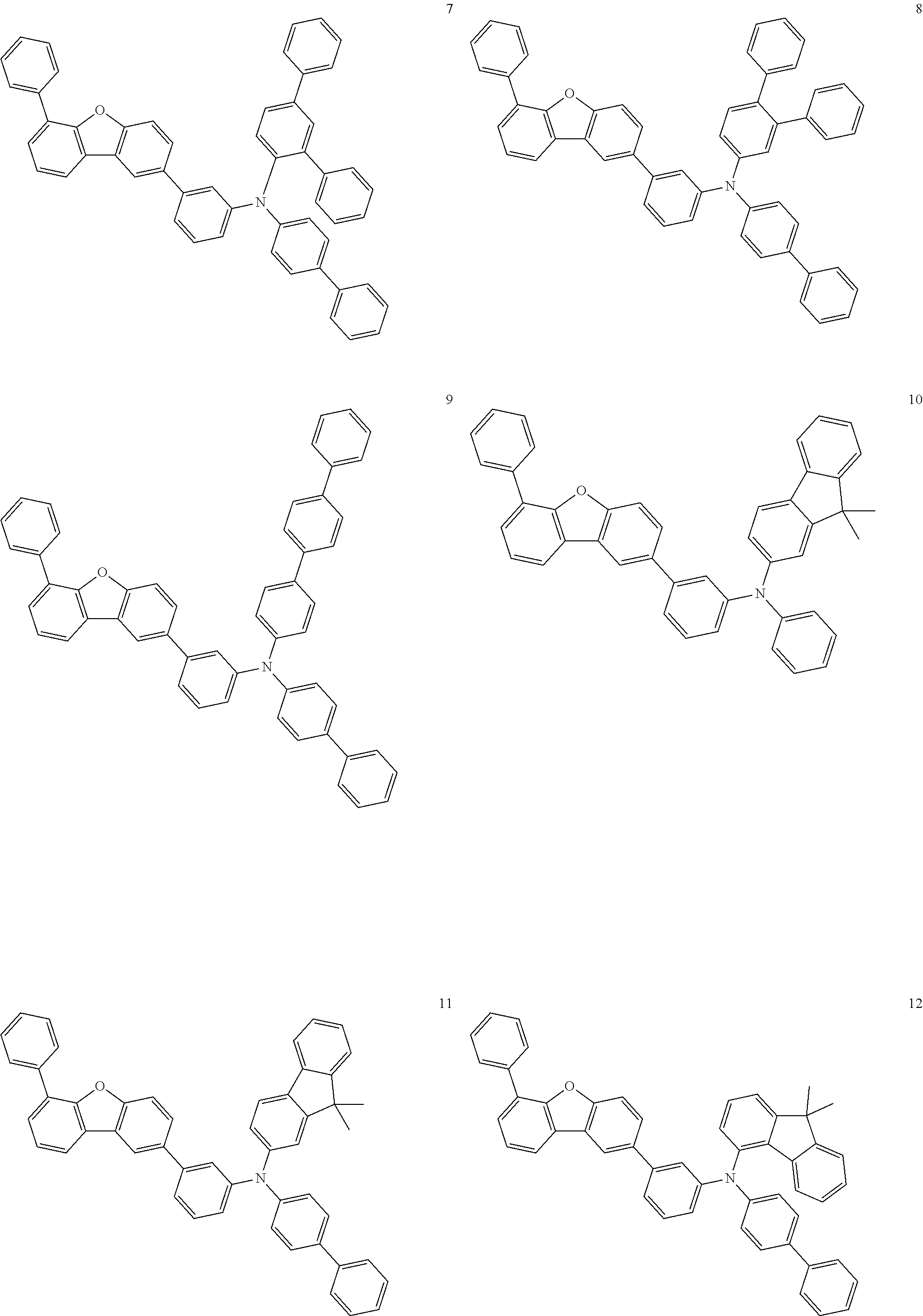

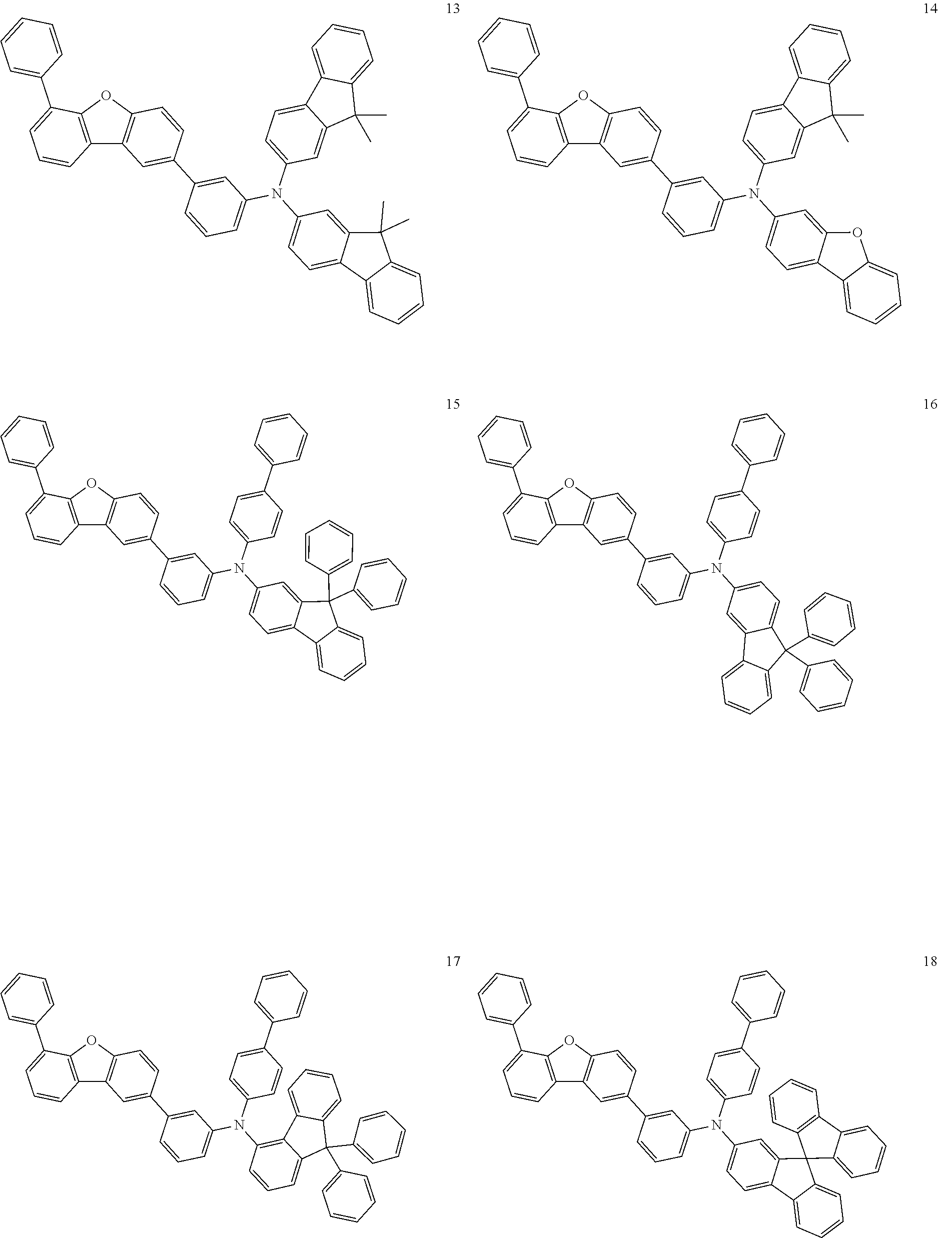

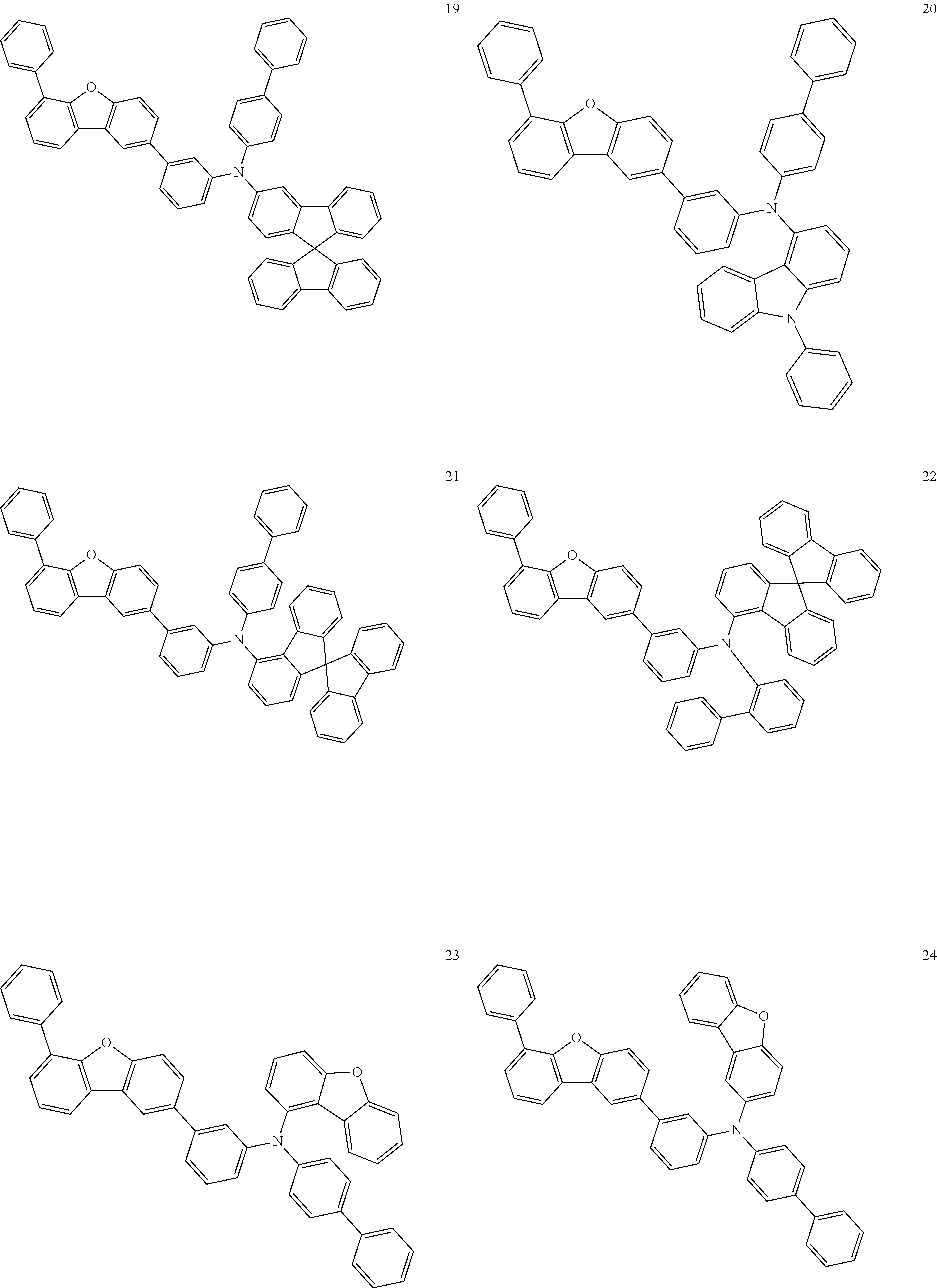

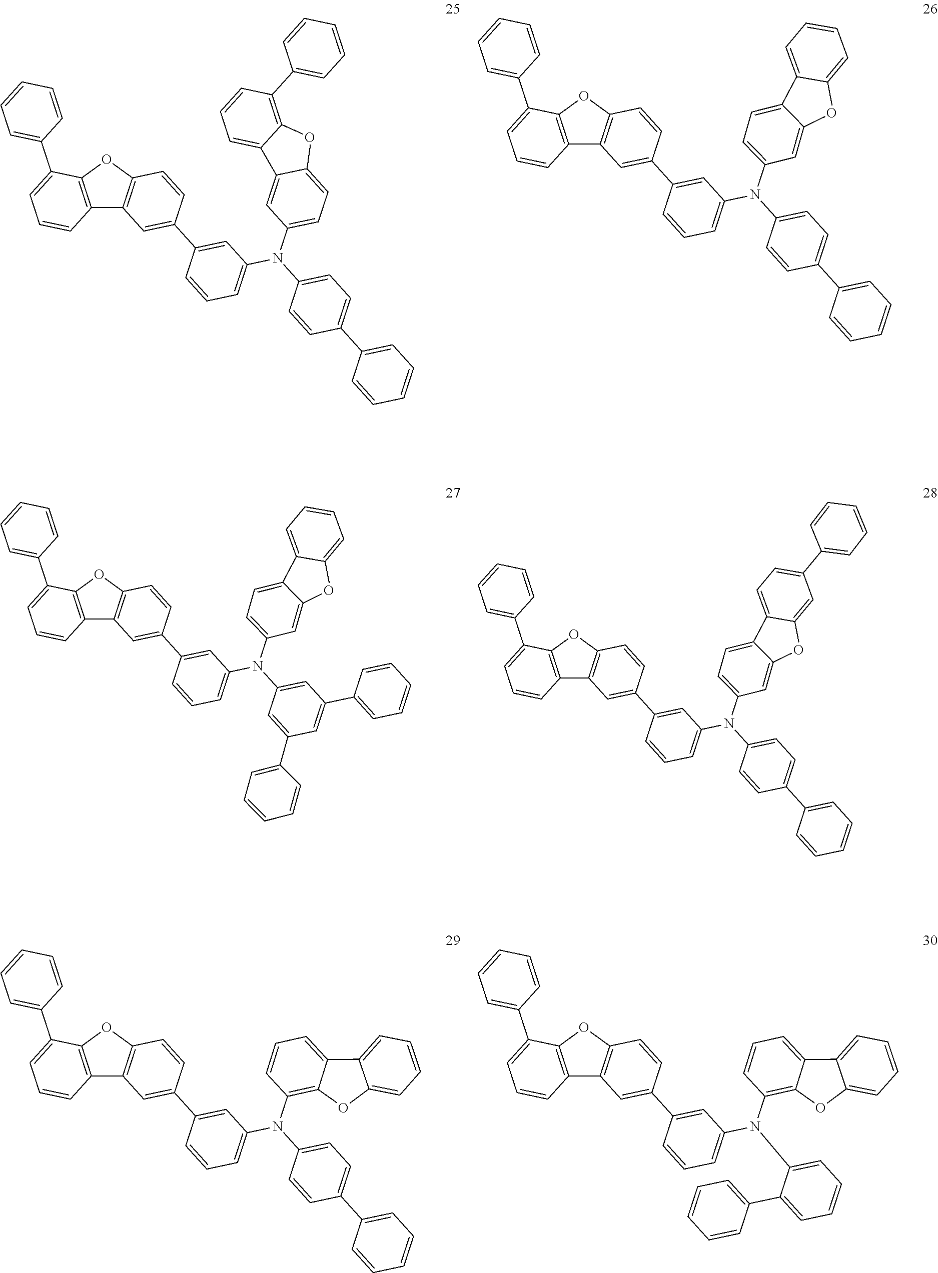

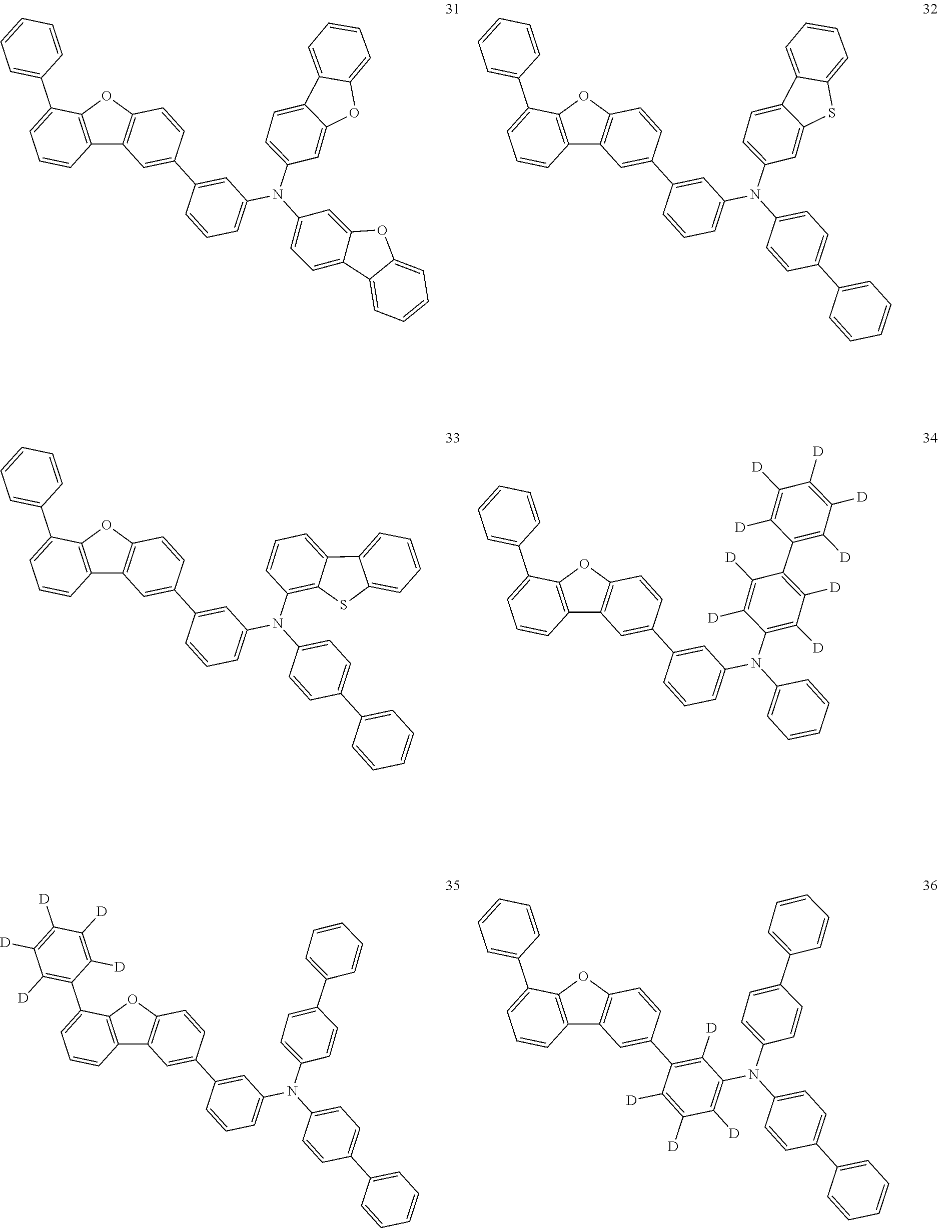

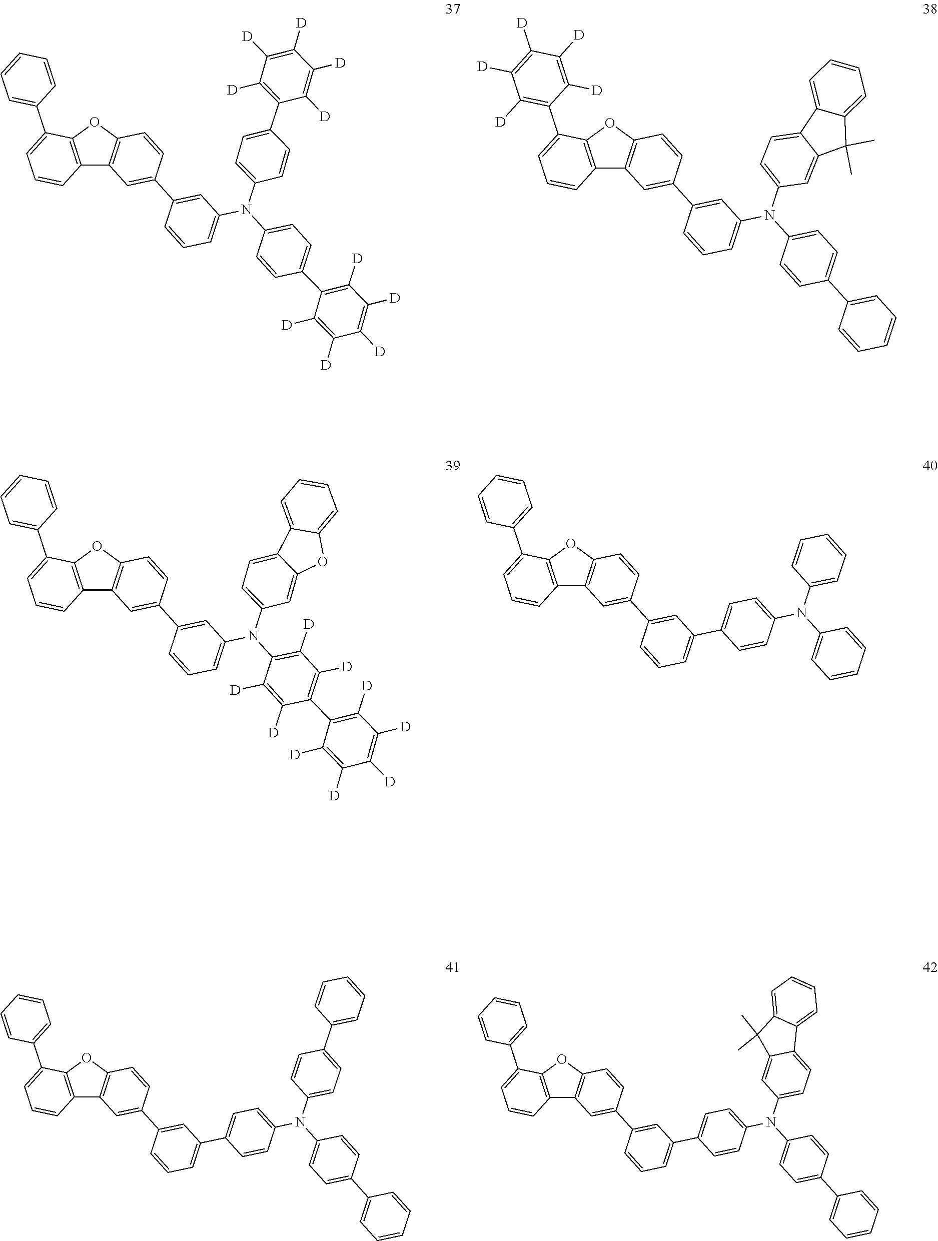

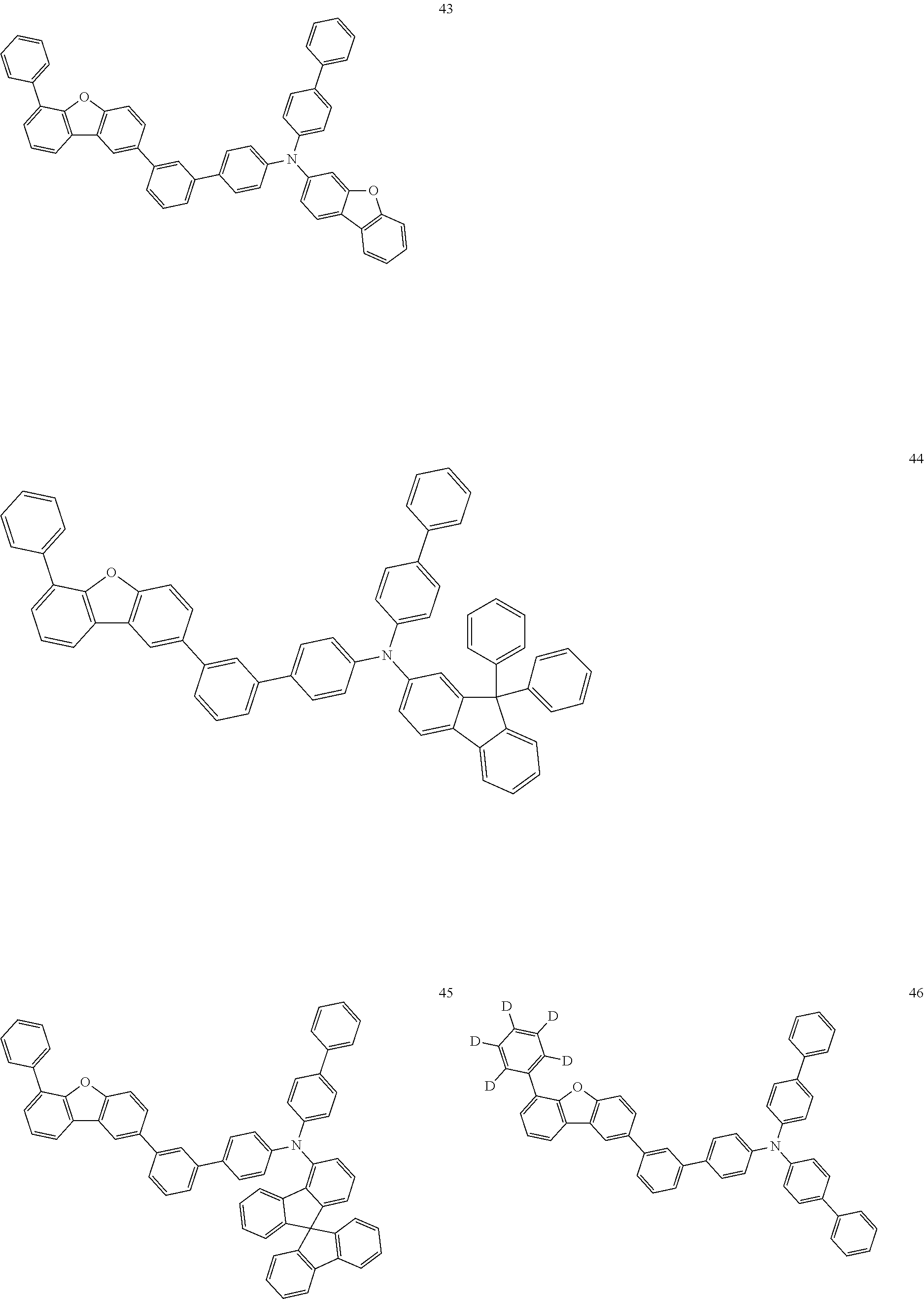 [SMILES: c1ccc(-c2ccc(N(c3ccc(-c4cccc(-c5ccc6oc7c(-c8ccccc8)cccc7c6c5)c4)cc3)c3ccc4c(c3)oc3ccccc34)cc2)cc1 ; c1ccc(-c2ccc(N(c3ccc(-c4cccc(-c5ccc6oc7c(-c8ccccc8)cccc7c6c5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6oc7c(-c8ccccc8)cccc7c6c5)c4)cc3)c3cccc4c3-c3ccccc3C43c4ccccc4-c4ccccc43)cc2)cc1 ; [2H]c1c([2H])c([2H])c(-c2cccc3c2oc2ccc(-c4cccc(-c5ccc(N(c6ccc(-c7ccccc7)cc6)c6ccc(-c7ccccc7)cc6)cc5)c4)cc23)c([2H])c1[2H]]

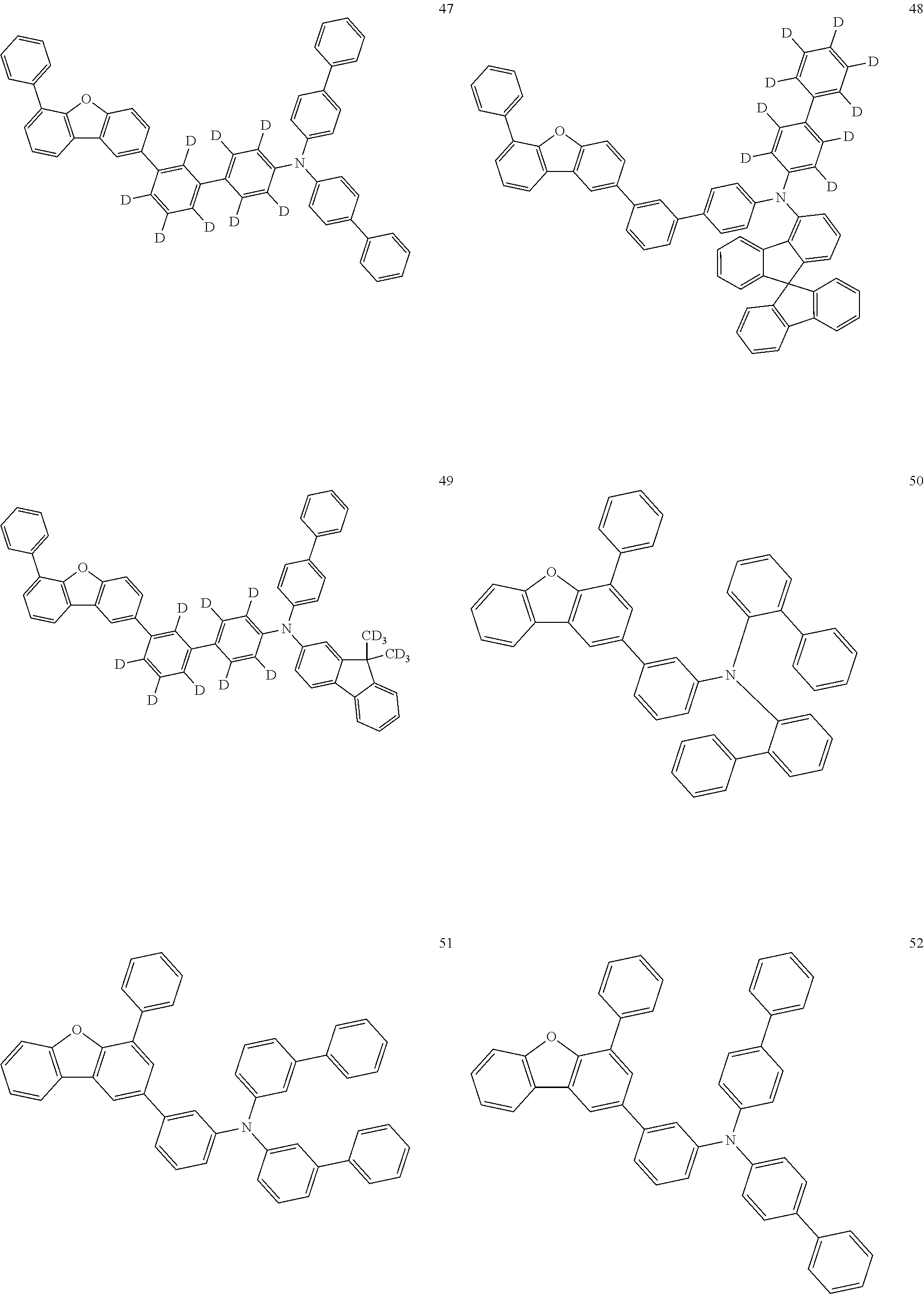

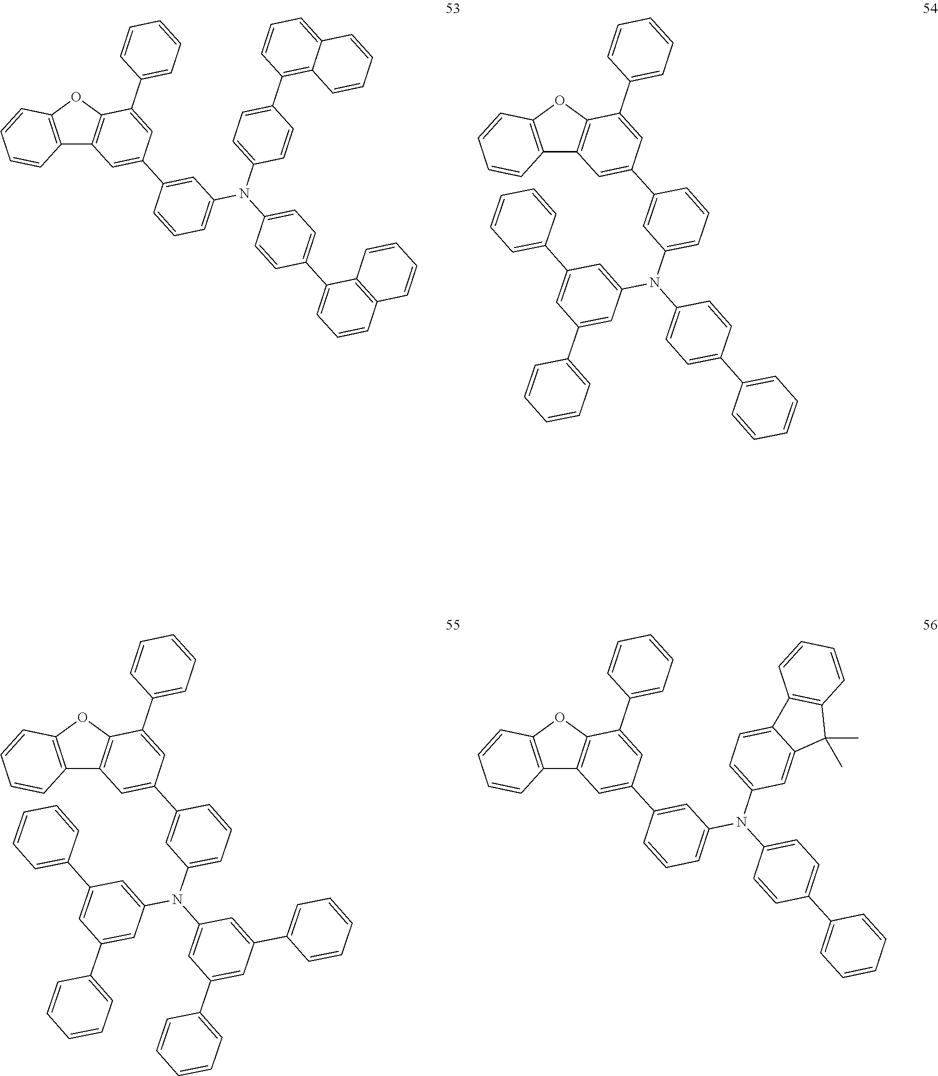 [SMILES: c1ccc(-c2cc(-c3cccc(N(c4ccc(-c5cccc6ccccc56)cc4)c4ccc(-c5cccc6ccccc56)cc4)c3)cc3c2oc2ccccc23)cc1 ; c1ccc(-c2ccc(N(c3cccc(-c4cc(-c5ccccc5)c5oc6ccccc6c5c4)c3)c3cc(-c4ccccc4)cc(-c4ccccc4)c3)cc2)cc1 ; c1ccc(-c2cc(-c3ccccc3)cc(N(c3cccc(-c4cc(-c5ccccc5)c5oc6ccccc6c5c4)c3)c3cc(-c4ccccc4)cc(-c4ccccc4)c3)c2)cc1 ; CC1(C)c2ccccc2-c2ccc(N(c3ccc(-c4ccccc4)cc3)c3cccc(-c4cc(-c5ccccc5)c5oc6ccccc6c5c4)c3)cc21]

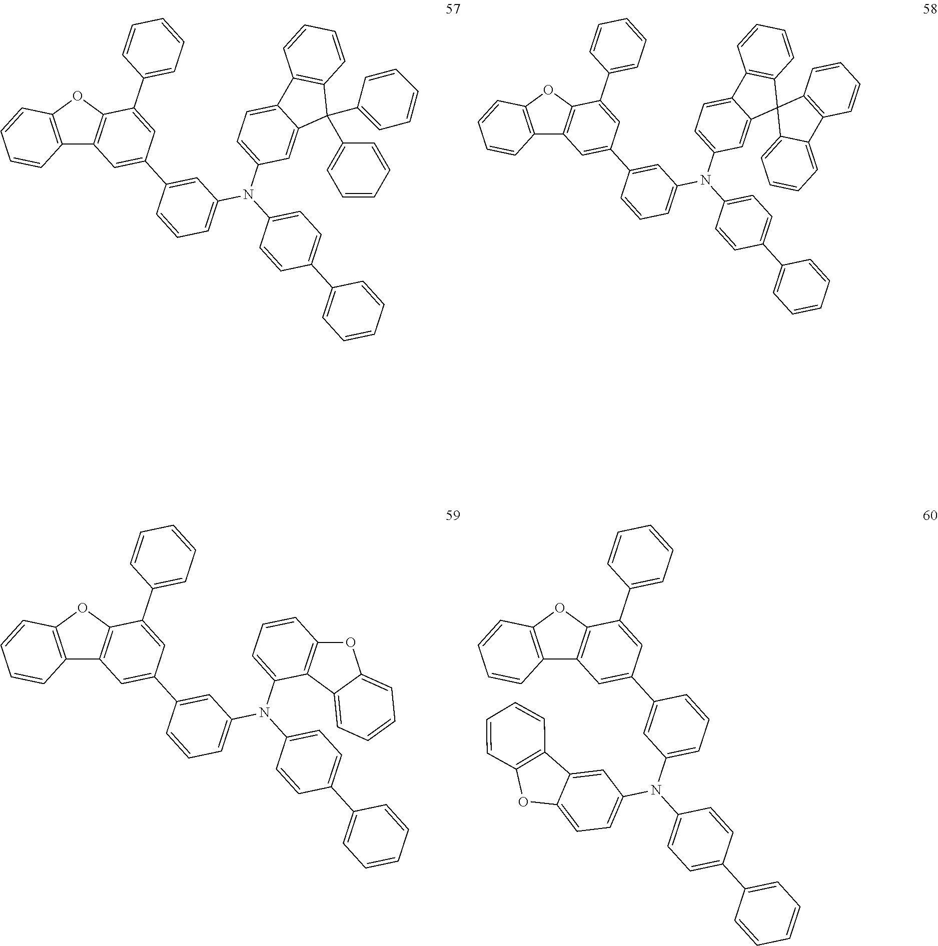 [SMILES: c1ccc(-c2ccc(N(c3cccc(-c4cc(-c5ccccc5)c5oc6ccccc6c5c4)c3)c3ccc4c(c3)C(c3ccccc3)(c3ccccc3)c3ccccc3-4)cc2)cc1 ; c1ccc(-c2ccc(N(c3cccc(-c4cc(-c5ccccc5)c5oc6ccccc6c5c4)c3)c3ccc4c(c3)C3(c5ccccc5-c5ccccc53)c3ccccc3-4)cc2)cc1 ; c1ccc(-c2ccc(N(c3cccc(-c4cc(-c5ccccc5)c5oc6ccccc6c5c4)c3)c3cccc4oc5ccccc5c34)cc2)cc1 ; c1ccc(-c2ccc(N(c3cccc(-c4cc(-c5ccccc5)c5oc6ccccc6c5c4)c3)c3ccc4oc5ccccc5c4c3)cc2)cc1]

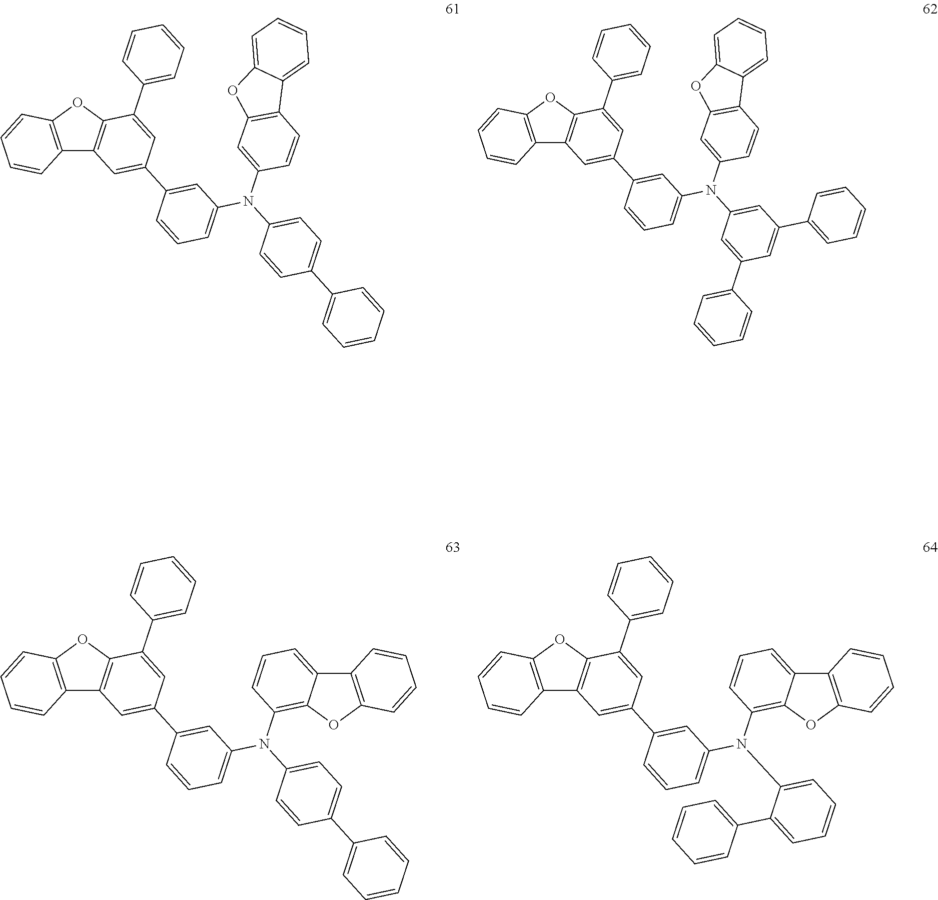 [SMILES: c1ccc(-c2ccc(N(c3cccc(-c4cc(-c5ccccc5)c5oc6ccccc6c5c4)c3)c3ccc4c(c3)oc3ccccc34)cc2)cc1 ; c1ccc(-c2cc(-c3ccccc3)cc(N(c3cccc(-c4cc(-c5ccccc5)c5oc6ccccc6c5c4)c3)c3ccc4c(c3)oc3ccccc34)c2)cc1 ; c1ccc(-c2ccc(N(c3cccc(-c4cc(-c5ccccc5)c5oc6ccccc6c5c4)c3)c3cccc4c3oc3ccccc34)cc2)cc1 ; c1ccc(-c2ccccc2N(c2cccc(-c3cc(-c4ccccc4)c4oc5ccccc5c4c3)c2)c2cccc3c2oc2ccccc23)cc1]

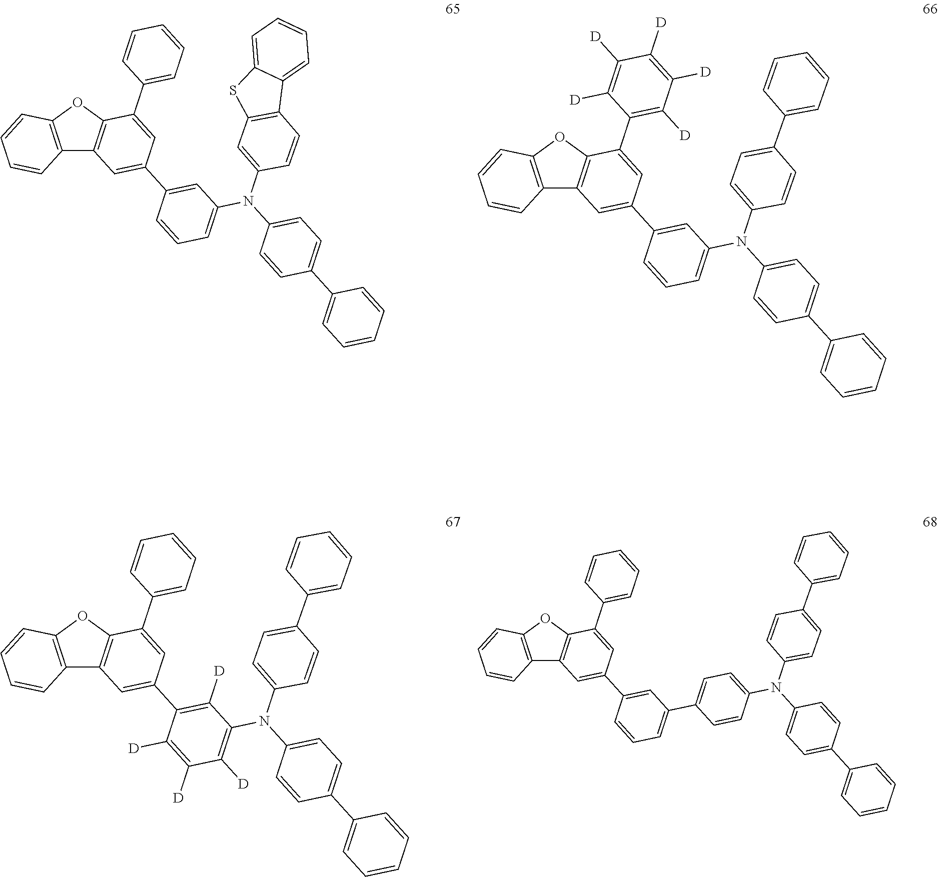 [SMILES: c1ccc(-c2ccc(N(c3cccc(-c4cc(-c5ccccc5)c5oc6ccccc6c5c4)c3)c3ccc4c(c3)sc3ccccc34)cc2)cc1 ; [2H]c1c([2H])c([2H])c(-c2cc(-c3cccc(N(c4ccc(-c5ccccc5)cc4)c4ccc(-c5ccccc5)cc4)c3)cc3c2oc2ccccc23)c([2H])c1[2H] ; [2H]c1c([2H])c(-c2cc(-c3ccccc3)c3oc4ccccc4c3c2)c([2H])c(N(c2ccc(-c3ccccc3)cc2)c2ccc(-c3ccccc3)cc2)c1[2H] ; c1ccc(-c2ccc(N(c3ccc(-c4ccccc4)cc3)c3ccc(-c4cccc(-c5cc(-c6ccccc6)c6oc7ccccc7c6c5)c4)cc3)cc2)cc1]

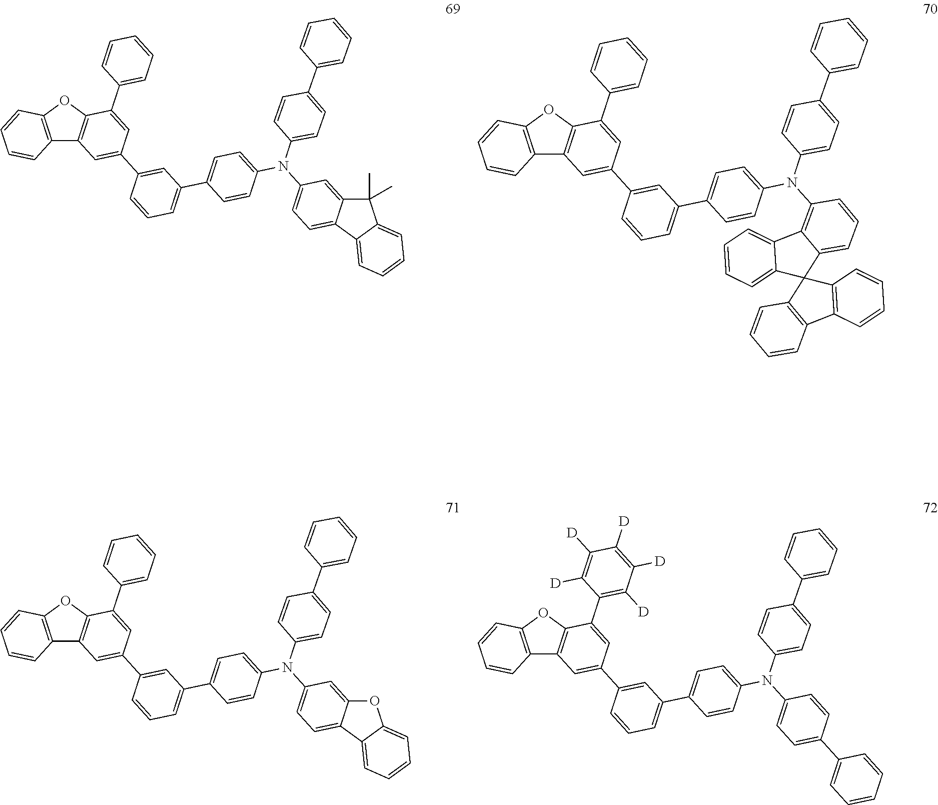 [SMILES: CC1(C)c2ccccc2-c2ccc(N(c3ccc(-c4ccccc4)cc3)c3ccc(-c4cccc(-c5cc(-c6ccccc6)c6oc7ccccc7c6c5)c4)cc3)cc21 ; c1ccc(-c2ccc(N(c3ccc(-c4cccc(-c5cc(-c6ccccc6)c6oc7ccccc7c6c5)c4)cc3)c3cccc4c3-c3ccccc3C43c4ccccc4-c4ccccc43)cc2)cc1 ; c1ccc(-c2ccc(N(c3ccc(-c4cccc(-c5cc(-c6ccccc6)c6oc7ccccc7c6c5)c4)cc3)c3ccc4c(c3)oc3ccccc34)cc2)cc1 ; [2H]c1c([2H])c([2H])c(-c2cc(-c3cccc(-c4ccc(N(c5ccc(-c6ccccc6)cc5)c5ccc(-c6ccccc6)cc5)cc4)c3)cc3c2oc2ccccc23)c([2H])c1[2H]]

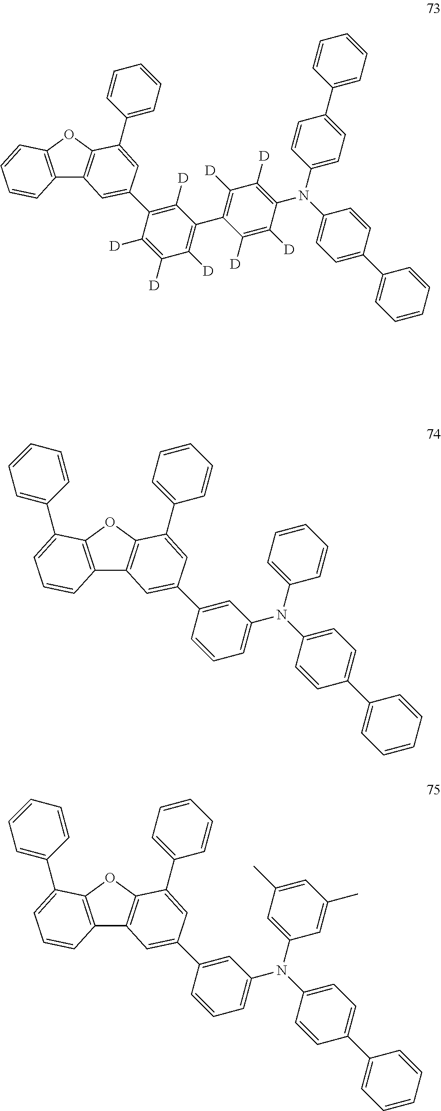 [SMILES: [2H]c1c([2H])c(-c2cc(-c3ccccc3)c3oc4ccccc4c3c2)c([2H])c(-c2c([2H])c([2H])c(N(c3ccc(-c4ccccc4)cc3)c3ccc(-c4ccccc4)cc3)c([2H])c2[2H])c1[2H] ; c1ccc(-c2ccc(N(c3ccccc3)c3cccc(-c4cc(-c5ccccc5)c5oc6c(-c7ccccc7)cccc6c5c4)c3)cc2)cc1 ; Cc1cc(C)cc(N(c2ccc(-c3ccccc3)cc2)c2cccc(-c3cc(-c4ccccc4)c4oc5c(-c6ccccc6)cccc5c4c3)c2)c1]

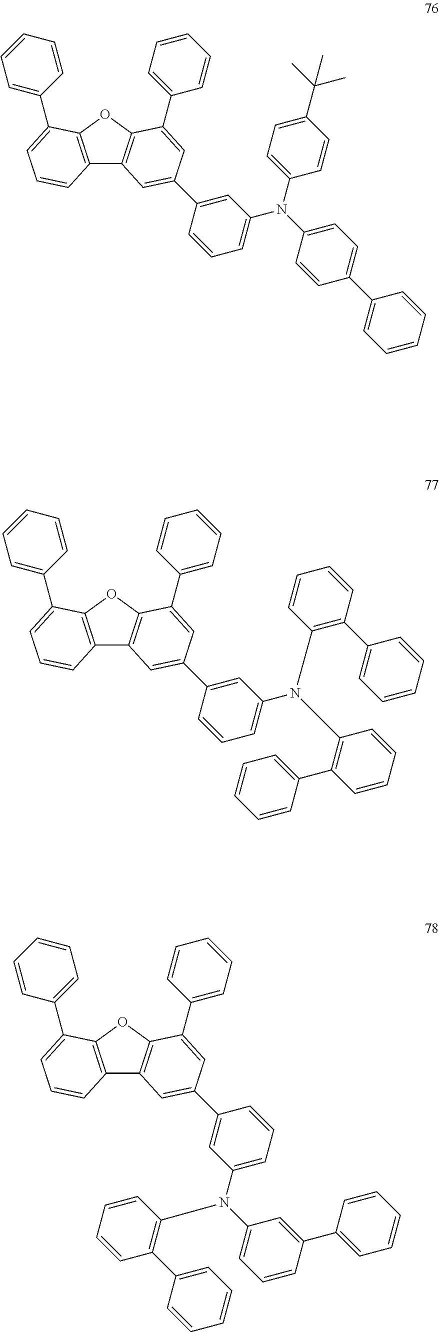 [SMILES: CC(C)(C)c1ccc(N(c2ccc(-c3ccccc3)cc2)c2cccc(-c3cc(-c4ccccc4)c4oc5c(-c6ccccc6)cccc5c4c3)c2)cc1 ; c1ccc(-c2ccccc2N(c2cccc(-c3cc(-c4ccccc4)c4oc5c(-c6ccccc6)cccc5c4c3)c2)c2ccccc2-c2ccccc2)cc1 ; c1ccc(-c2cccc(N(c3cccc(-c4cc(-c5ccccc5)c5oc6c(-c7ccccc7)cccc6c5c4)c3)c3ccccc3-c3ccccc3)c2)cc1]

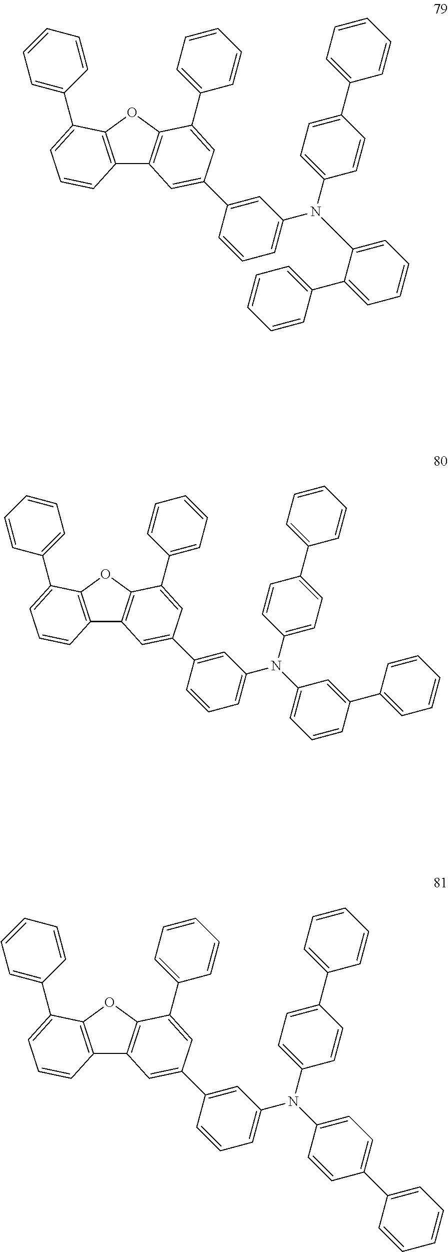 [SMILES: c1ccc(-c2ccc(N(c3cccc(-c4cc(-c5ccccc5)c5oc6c(-c7ccccc7)cccc6c5c4)c3)c3ccccc3-c3ccccc3)cc2)cc1 ; c1ccc(-c2ccc(N(c3cccc(-c4ccccc4)c3)c3cccc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cccc(-c4cc(-c5ccccc5)c5oc6c(-c7ccccc7)cccc6c5c4)c3)cc2)cc1]

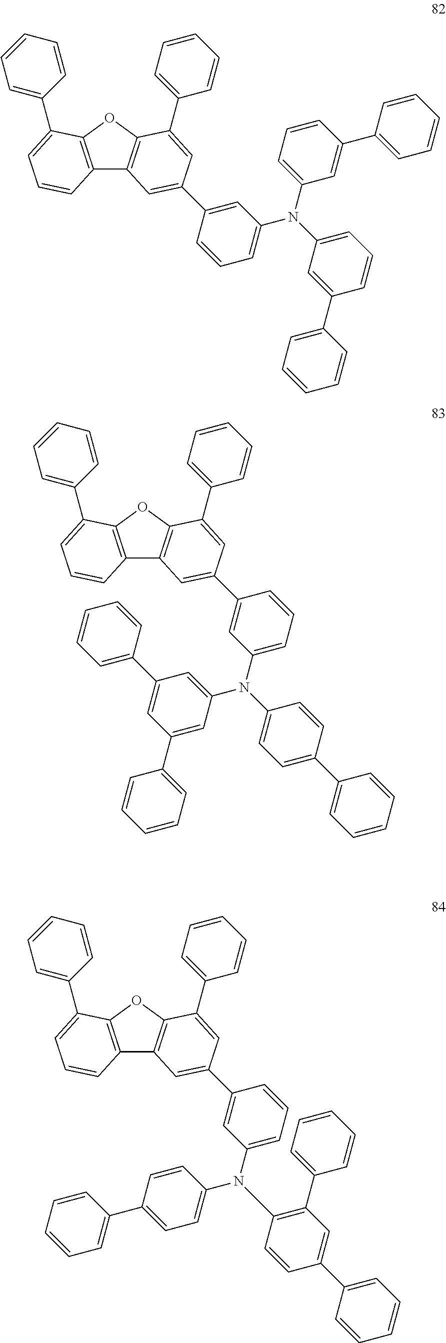 [SMILES: c1ccc(-c2cccc(N(c3cccc(-c4ccccc4)c3)c3cccc(-c4cc(-c5ccccc5)c5oc6c(-c7ccccc7)cccc6c5c4)c3)c2)cc1 ; c1ccc(-c2ccc(N(c3cccc(-c4cc(-c5ccccc5)c5oc6c(-c7ccccc7)cccc6c5c4)c3)c3cc(-c4ccccc4)cc(-c4ccccc4)c3)cc2)cc1 ; c1ccc(-c2ccc(N(c3cccc(-c4cc(-c5ccccc5)c5oc6c(-c7ccccc7)cccc6c5c4)c3)c3ccc(-c4ccccc4)cc3-c3ccccc3)cc2)cc1]

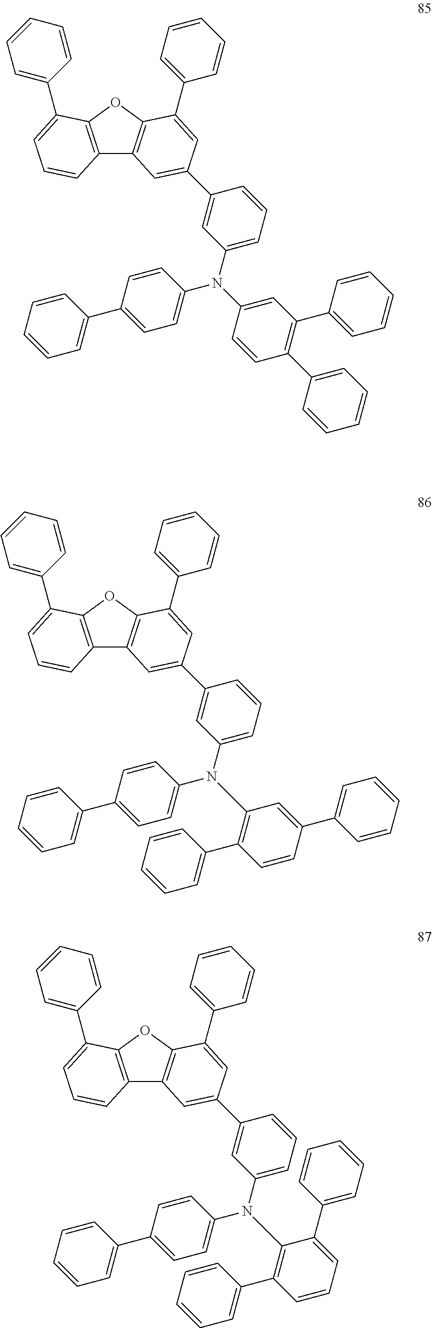 [SMILES: c1ccc(-c2ccc(N(c3cccc(-c4cc(-c5ccccc5)c5oc6c(-c7ccccc7)cccc6c5c4)c3)c3ccc(-c4ccccc4)c(-c4ccccc4)c3)cc2)cc1 ; c1ccc(-c2ccc(N(c3cccc(-c4cc(-c5ccccc5)c5oc6c(-c7ccccc7)cccc6c5c4)c3)c3cc(-c4ccccc4)ccc3-c3ccccc3)cc2)cc1 ; c1ccc(-c2ccc(N(c3cccc(-c4cc(-c5ccccc5)c5oc6c(-c7ccccc7)cccc6c5c4)c3)c3c(-c4ccccc4)cccc3-c3ccccc3)cc2)cc1]

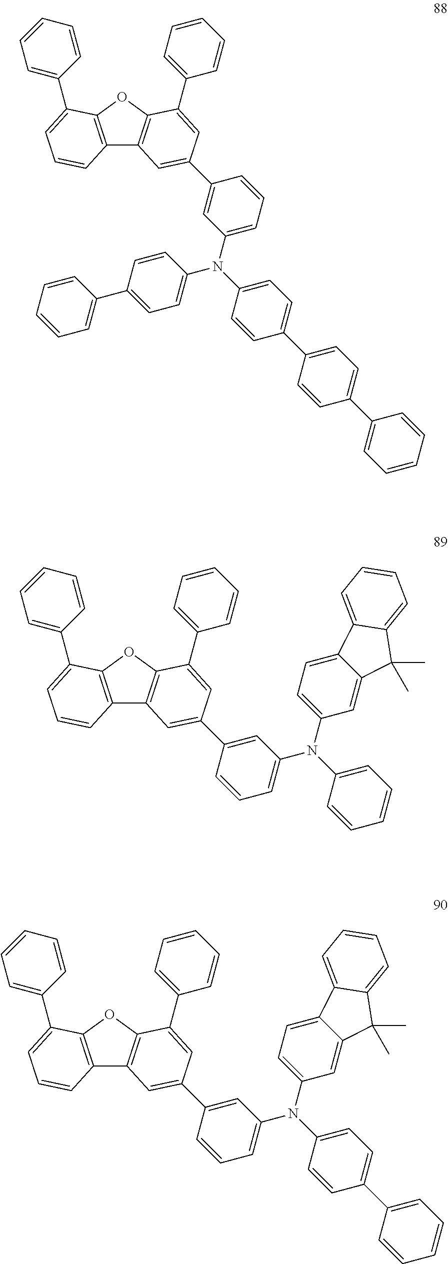 [SMILES: c1ccc(-c2ccc(-c3ccc(N(c4ccc(-c5ccccc5)cc4)c4cccc(-c5cc(-c6ccccc6)c6oc7c(-c8ccccc8)cccc7c6c5)c4)cc3)cc2)cc1 ; CC1(C)c2ccccc2-c2ccc(N(c3ccccc3)c3cccc(-c4cc(-c5ccccc5)c5oc6c(-c7ccccc7)cccc6c5c4)c3)cc21 ; CC1(C)c2ccccc2-c2ccc(N(c3ccc(-c4ccccc4)cc3)c3cccc(-c4cc(-c5ccccc5)c5oc6c(-c7ccccc7)cccc6c5c4)c3)cc21]

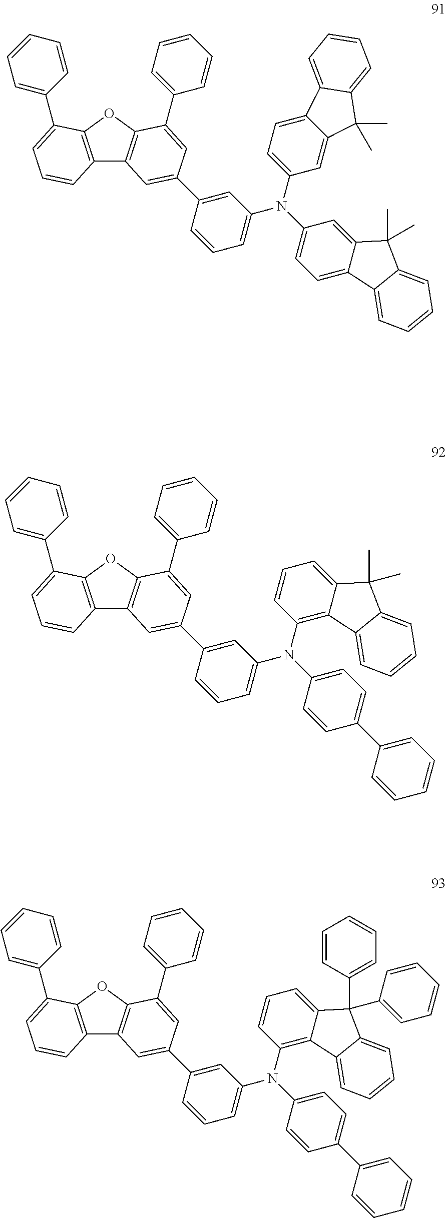 [SMILES: CC1(C)c2ccccc2-c2ccc(N(c3cccc(-c4cc(-c5ccccc5)c5oc6c(-c7ccccc7)cccc6c5c4)c3)c3ccc4c(c3)C(C)(C)c3ccccc3-4)cc21 ; CC1(C)c2ccccc2-c2c(N(c3ccc(-c4ccccc4)cc3)c3cccc(-c4cc(-c5ccccc5)c5oc6c(-c7ccccc7)cccc6c5c4)c3)cccc21 ; c1ccc(-c2ccc(N(c3cccc(-c4cc(-c5ccccc5)c5oc6c(-c7ccccc7)cccc6c5c4)c3)c3cccc4c3-c3ccccc3C4(c3ccccc3)c3ccccc3)cc2)cc1]

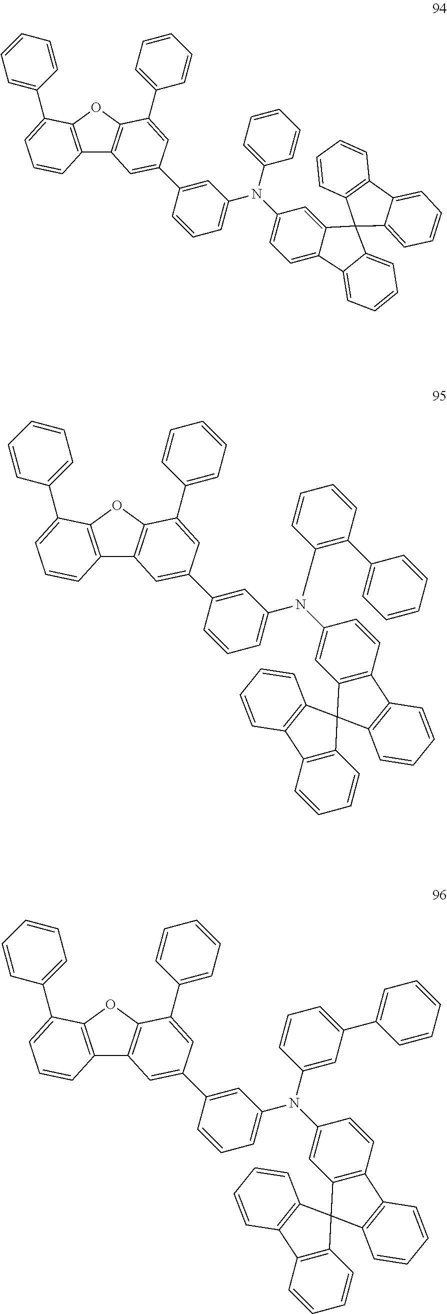 [SMILES: c1ccc(-c2cccc3c2oc2c(-c4ccccc4)cc(-c4cccc(N(c5ccccc5)c5ccc6c(c5)C5(c7ccccc7-c7ccccc75)c5ccccc5-6)c4)cc23)cc1 ; c1ccc(-c2ccccc2N(c2cccc(-c3cc(-c4ccccc4)c4oc5c(-c6ccccc6)cccc5c4c3)c2)c2ccc3c(c2)C2(c4ccccc4-c4ccccc42)c2ccccc2-3)cc1 ; c1ccc(-c2cccc(N(c3cccc(-c4cc(-c5ccccc5)c5oc6c(-c7ccccc7)cccc6c5c4)c3)c3ccc4c(c3)C3(c5ccccc5-c5ccccc53)c3ccccc3-4)c2)cc1]

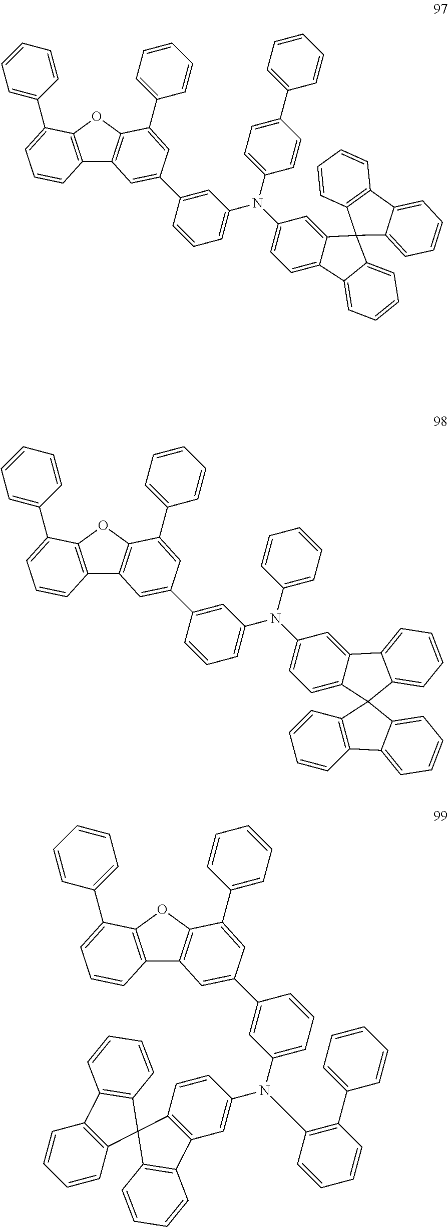 [SMILES: c1ccc(-c2ccc(N(c3cccc(-c4cc(-c5ccccc5)c5oc6c(-c7ccccc7)cccc6c5c4)c3)c3ccc4c(c3)C3(c5ccccc5-c5ccccc53)c3ccccc3-4)cc2)cc1 ; c1ccc(-c2cccc3c2oc2c(-c4ccccc4)cc(-c4cccc(N(c5ccccc5)c5ccc6c(c5)-c5ccccc5C65c6ccccc6-c6ccccc65)c4)cc23)cc1 ; c1ccc(-c2ccccc2N(c2cccc(-c3cc(-c4ccccc4)c4oc5c(-c6ccccc6)cccc5c4c3)c2)c2ccc3c(c2)-c2ccccc2C32c3ccccc3-c3ccccc32)cc1]

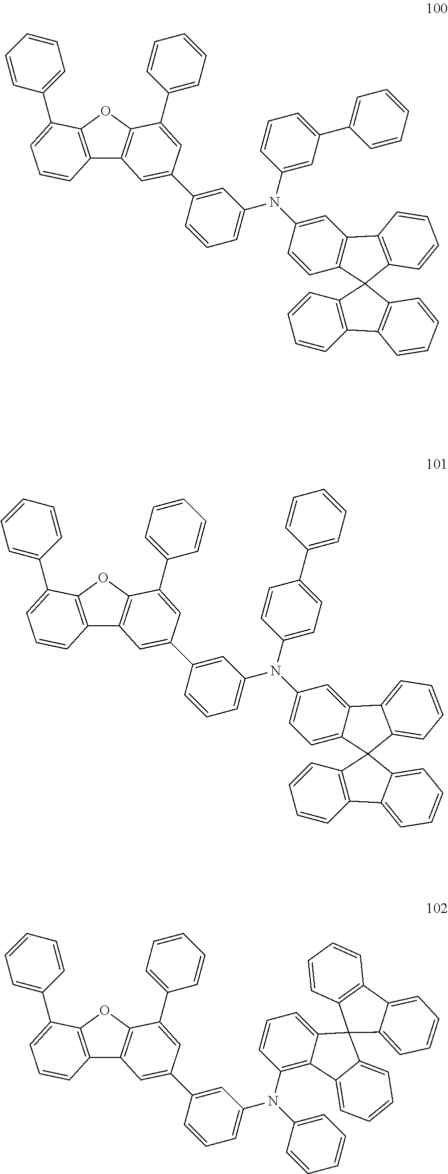 [SMILES: c1ccc(-c2cccc(N(c3cccc(-c4cc(-c5ccccc5)c5oc6c(-c7ccccc7)cccc6c5c4)c3)c3ccc4c(c3)-c3ccccc3C43c4ccccc4-c4ccccc43)c2)cc1 ; c1ccc(-c2ccc(N(c3cccc(-c4cc(-c5ccccc5)c5oc6c(-c7ccccc7)cccc6c5c4)c3)c3ccc4c(c3)-c3ccccc3C43c4ccccc4-c4ccccc43)cc2)cc1 ; c1ccc(-c2cccc3c2oc2c(-c4ccccc4)cc(-c4cccc(N(c5ccccc5)c5cccc6c5-c5ccccc5C65c6ccccc6-c6ccccc65)c4)cc23)cc1]

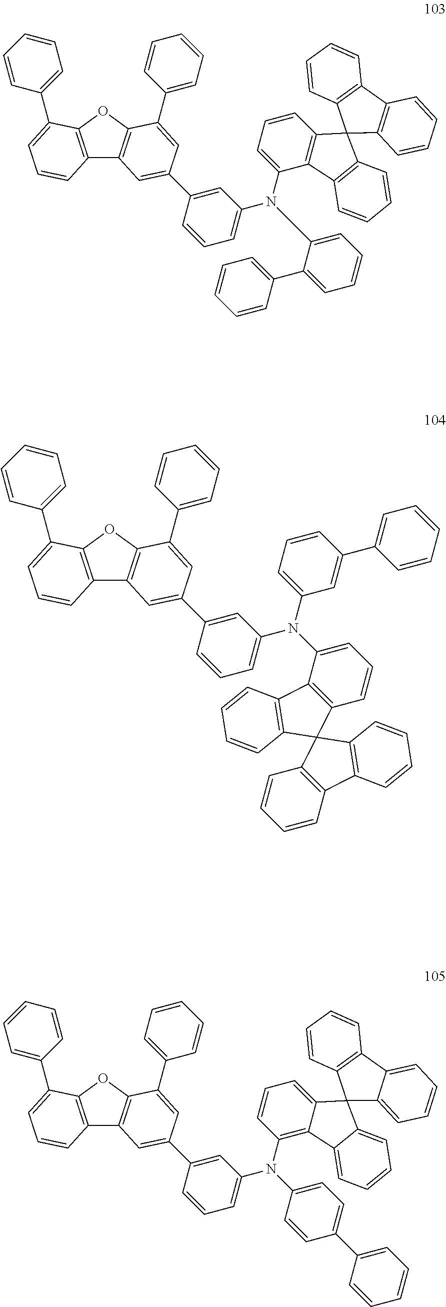 [SMILES: c1ccc(-c2ccccc2N(c2cccc(-c3cc(-c4ccccc4)c4oc5c(-c6ccccc6)cccc5c4c3)c2)c2cccc3c2-c2ccccc2C32c3ccccc3-c3ccccc32)cc1 ; c1ccc(-c2cccc(N(c3cccc(-c4cc(-c5ccccc5)c5oc6c(-c7ccccc7)cccc6c5c4)c3)c3cccc4c3-c3ccccc3C43c4ccccc4-c4ccccc43)c2)cc1 ; c1ccc(-c2ccc(N(c3cccc(-c4cc(-c5ccccc5)c5oc6c(-c7ccccc7)cccc6c5c4)c3)c3cccc4c3-c3ccccc3C43c4ccccc4-c4ccccc43)cc2)cc1]

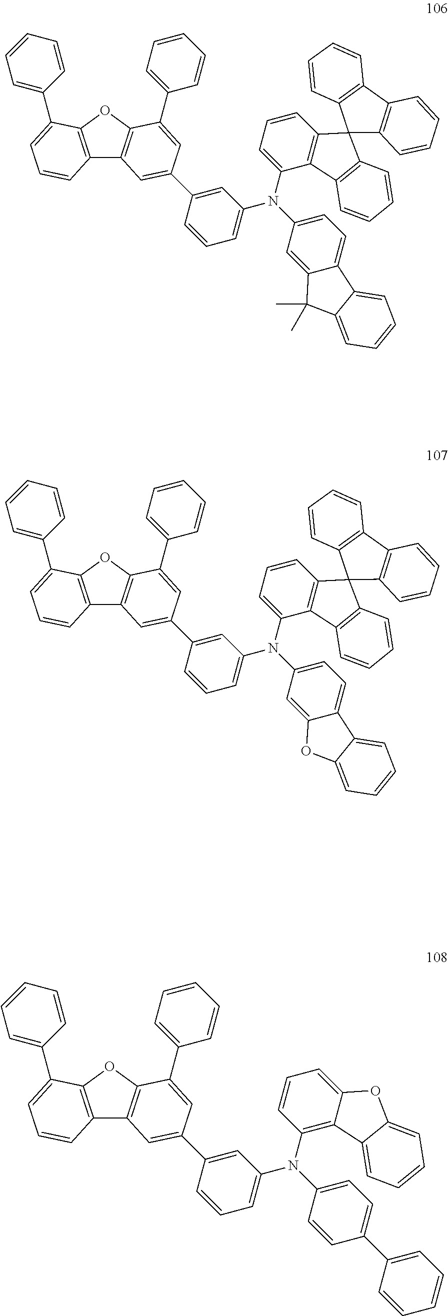 [SMILES: CC1(C)c2ccccc2-c2ccc(N(c3cccc(-c4cc(-c5ccccc5)c5oc6c(-c7ccccc7)cccc6c5c4)c3)c3cccc4c3-c3ccccc3C43c4ccccc4-c4ccccc43)cc21 ; c1ccc(-c2cccc3c2oc2c(-c4ccccc4)cc(-c4cccc(N(c5ccc6c(c5)oc5ccccc56)c5cccc6c5-c5ccccc5C65c6ccccc6-c6ccccc65)c4)cc23)cc1 ; c1ccc(-c2ccc(N(c3cccc(-c4cc(-c5ccccc5)c5oc6c(-c7ccccc7)cccc6c5c4)c3)c3cccc4oc5ccccc5c34)cc2)cc1]

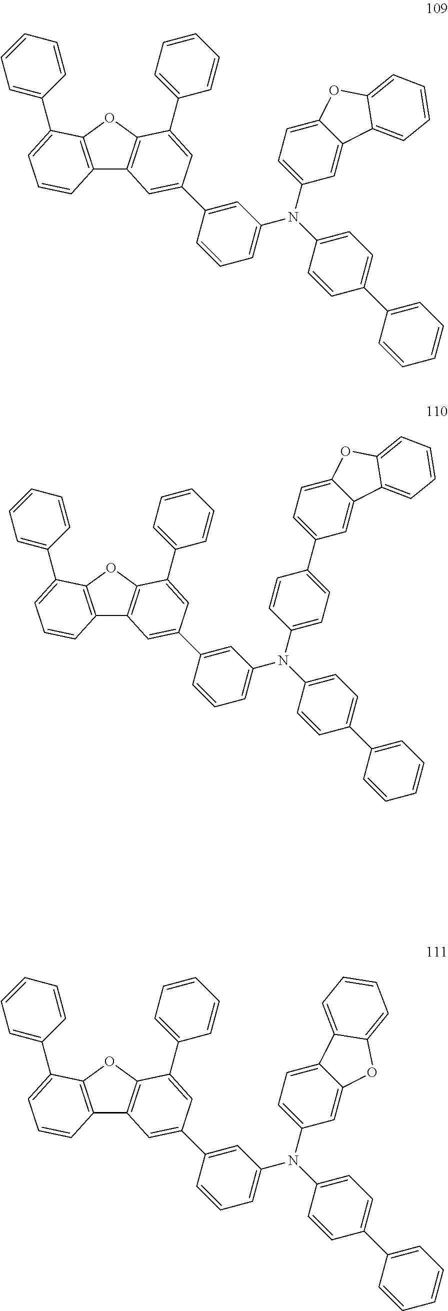 [SMILES: c1ccc(-c2ccc(N(c3cccc(-c4cc(-c5ccccc5)c5oc6c(-c7ccccc7)cccc6c5c4)c3)c3ccc4oc5ccccc5c4c3)cc2)cc1 ; c1ccc(-c2ccc(N(c3ccc(-c4ccc5oc6ccccc6c5c4)cc3)c3cccc(-c4cc(-c5ccccc5)c5oc6c(-c7ccccc7)cccc6c5c4)c3)cc2)cc1 ; c1ccc(-c2ccc(N(c3cccc(-c4cc(-c5ccccc5)c5oc6c(-c7ccccc7)cccc6c5c4)c3)c3ccc4c(c3)oc3ccccc34)cc2)cc1]

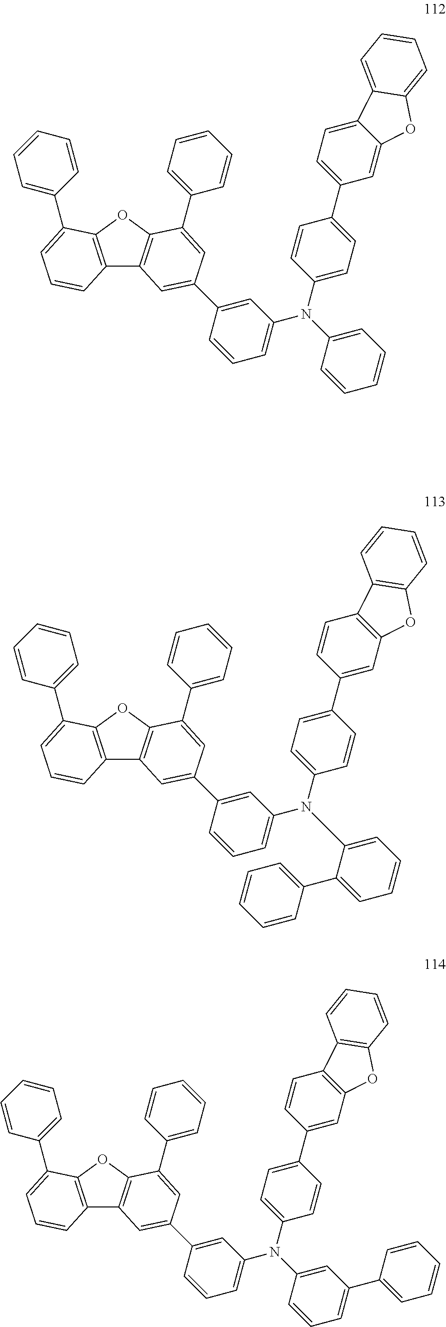 [SMILES: c1ccc(-c2cccc3c2oc2c(-c4ccccc4)cc(-c4cccc(N(c5ccccc5)c5ccc(-c6ccc7c(c6)oc6ccccc67)cc5)c4)cc23)cc1 ; c1ccc(-c2ccccc2N(c2ccc(-c3ccc4c(c3)oc3ccccc34)cc2)c2cccc(-c3cc(-c4ccccc4)c4oc5c(-c6ccccc6)cccc5c4c3)c2)cc1 ; c1ccc(-c2cccc(N(c3ccc(-c4ccc5c(c4)oc4ccccc45)cc3)c3cccc(-c4cc(-c5ccccc5)c5oc6c(-c7ccccc7)cccc6c5c4)c3)c2)cc1]

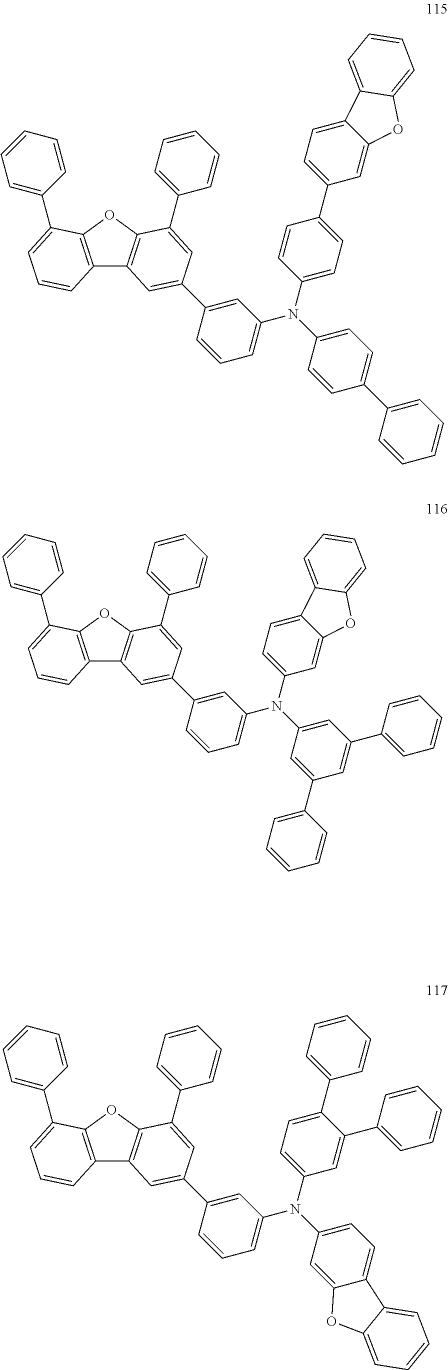 [SMILES: c1ccc(-c2ccc(N(c3ccc(-c4ccc5c(c4)oc4ccccc45)cc3)c3cccc(-c4cc(-c5ccccc5)c5oc6c(-c7ccccc7)cccc6c5c4)c3)cc2)cc1 ; c1ccc(-c2cc(-c3ccccc3)cc(N(c3cccc(-c4cc(-c5ccccc5)c5oc6c(-c7ccccc7)cccc6c5c4)c3)c3ccc4c(c3)oc3ccccc34)c2)cc1 ; c1ccc(-c2ccc(N(c3cccc(-c4cc(-c5ccccc5)c5oc6c(-c7ccccc7)cccc6c5c4)c3)c3ccc4c(c3)oc3ccccc34)cc2-c2ccccc2)cc1]

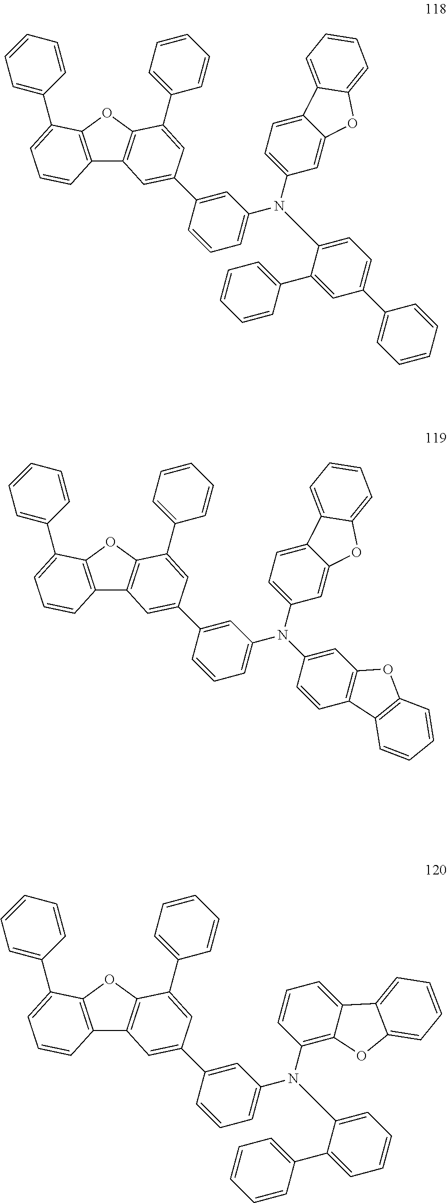 [SMILES: c1ccc(-c2ccc(N(c3cccc(-c4cc(-c5ccccc5)c5oc6c(-c7ccccc7)cccc6c5c4)c3)c3ccc4c(c3)oc3ccccc34)c(-c3ccccc3)c2)cc1 ; c1ccc(-c2cccc3c2oc2c(-c4ccccc4)cc(-c4cccc(N(c5ccc6c(c5)oc5ccccc56)c5ccc6c(c5)oc5ccccc56)c4)cc23)cc1 ; c1ccc(-c2ccccc2N(c2cccc(-c3cc(-c4ccccc4)c4oc5c(-c6ccccc6)cccc5c4c3)c2)c2cccc3c2oc2ccccc23)cc1]

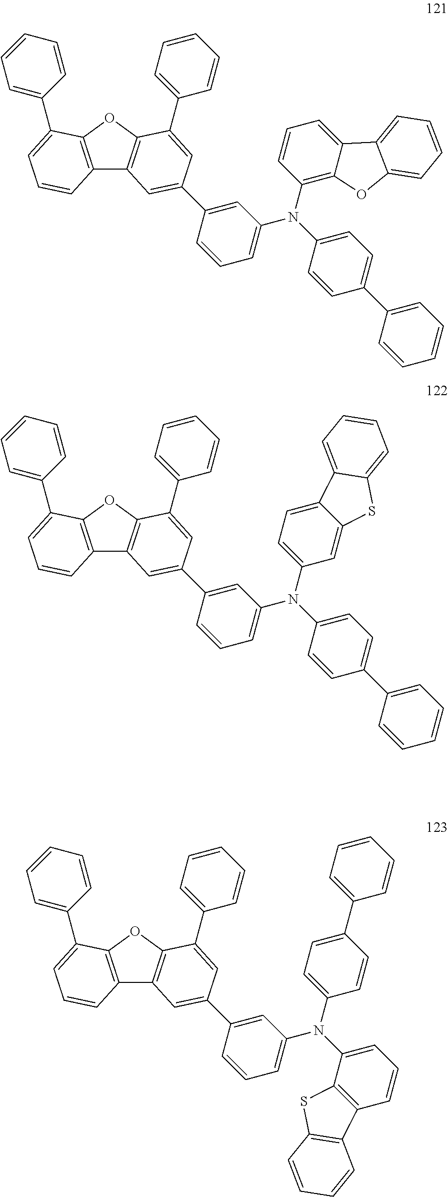 [SMILES: c1ccc(-c2ccc(N(c3cccc(-c4cc(-c5ccccc5)c5oc6c(-c7ccccc7)cccc6c5c4)c3)c3cccc4c3oc3ccccc34)cc2)cc1 ; c1ccc(-c2ccc(N(c3cccc(-c4cc(-c5ccccc5)c5oc6c(-c7ccccc7)cccc6c5c4)c3)c3ccc4c(c3)sc3ccccc34)cc2)cc1 ; c1ccc(-c2ccc(N(c3cccc(-c4cc(-c5ccccc5)c5oc6c(-c7ccccc7)cccc6c5c4)c3)c3cccc4c3sc3ccccc34)cc2)cc1]

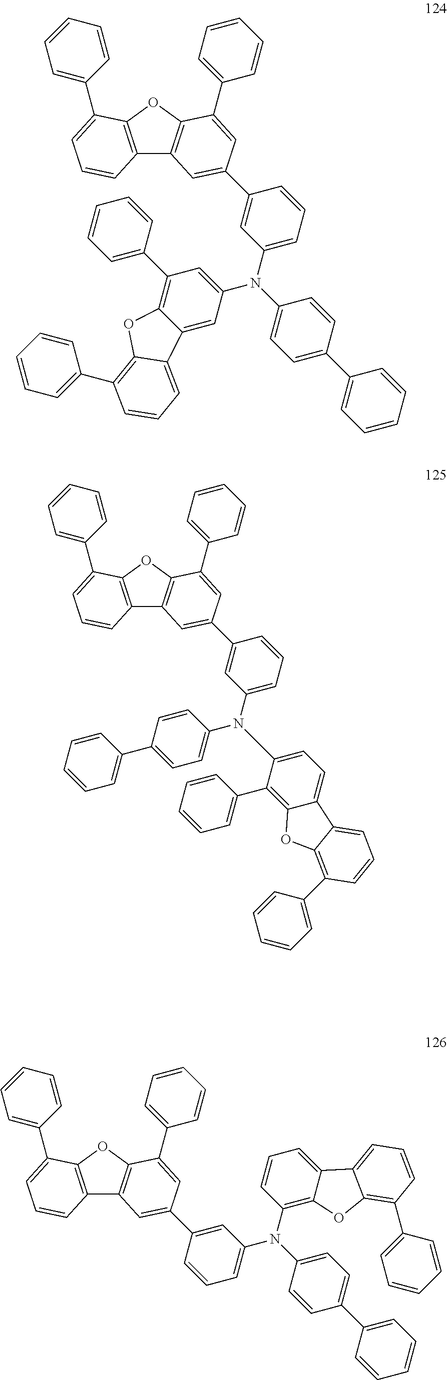 [SMILES: c1ccc(-c2ccc(N(c3cccc(-c4cc(-c5ccccc5)c5oc6c(-c7ccccc7)cccc6c5c4)c3)c3cc(-c4ccccc4)c4oc5c(-c6ccccc6)cccc5c4c3)cc2)cc1 ; c1ccc(-c2ccc(N(c3cccc(-c4cc(-c5ccccc5)c5oc6c(-c7ccccc7)cccc6c5c4)c3)c3ccc4c(oc5c(-c6ccccc6)cccc54)c3-c3ccccc3)cc2)cc1 ; c1ccc(-c2ccc(N(c3cccc(-c4cc(-c5ccccc5)c5oc6c(-c7ccccc7)cccc6c5c4)c3)c3cccc4c3oc3c(-c5ccccc5)cccc34)cc2)cc1]

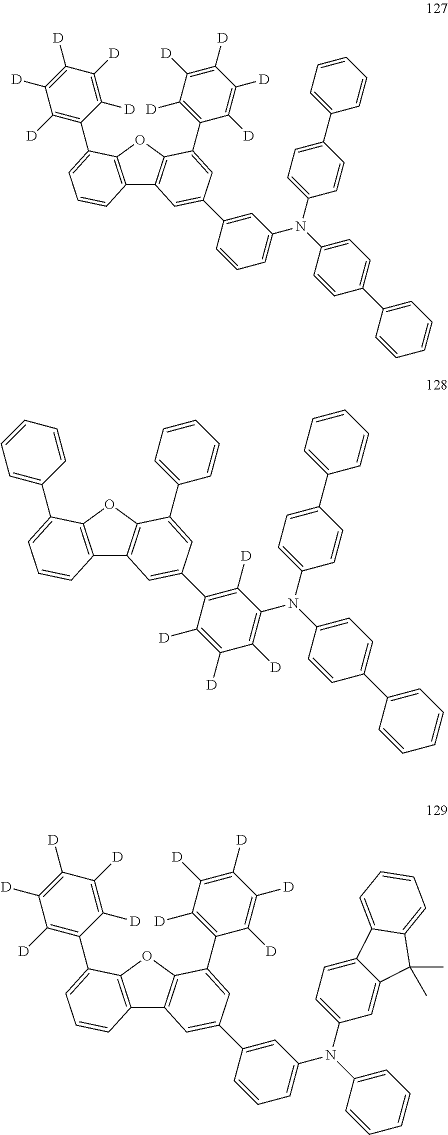 [SMILES: [2H]c1c([2H])c([2H])c(-c2cccc3c2oc2c(-c4c([2H])c([2H])c([2H])c([2H])c4[2H])cc(-c4cccc(N(c5ccc(-c6ccccc6)cc5)c5ccc(-c6ccccc6)cc5)c4)cc23)c([2H])c1[2H] ; [2H]c1c([2H])c(-c2cc(-c3ccccc3)c3oc4c(-c5ccccc5)cccc4c3c2)c([2H])c(N(c2ccc(-c3ccccc3)cc2)c2ccc(-c3ccccc3)cc2)c1[2H] ; [2H]c1c([2H])c([2H])c(-c2cccc3c2oc2c(-c4c([2H])c([2H])c([2H])c([2H])c4[2H])cc(-c4cccc(N(c5ccccc5)c5ccc6c(c5)C(C)(C)c5ccccc5-6)c4)cc23)c([2H])c1[2H]]

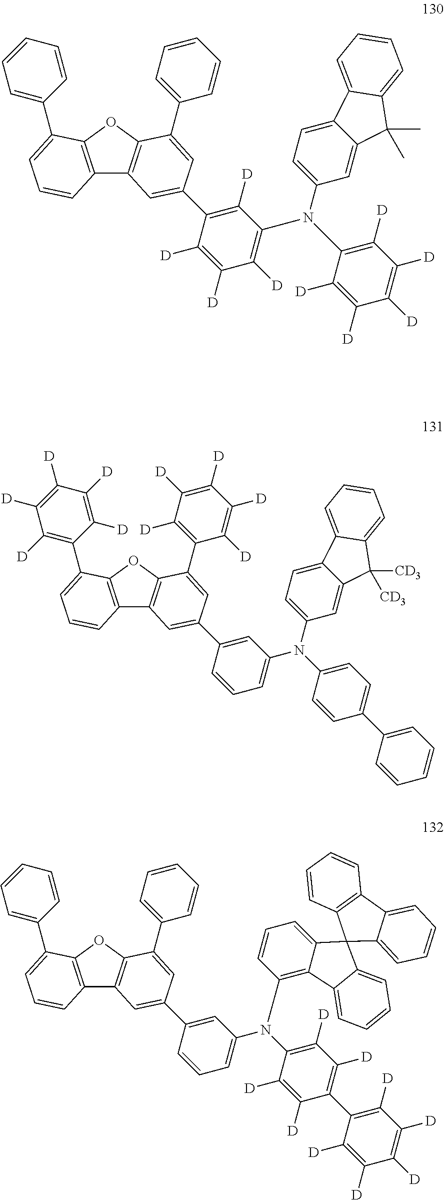 [SMILES: [2H]c1c([2H])c([2H])c(N(c2ccc3c(c2)C(C)(C)c2ccccc2-3)c2c([2H])c([2H])c([2H])c(-c3cc(-c4ccccc4)c4oc5c(-c6ccccc6)cccc5c4c3)c2[2H])c([2H])c1[2H] ; [2H]c1c([2H])c([2H])c(-c2cccc3c2oc2c(-c4c([2H])c([2H])c([2H])c([2H])c4[2H])cc(-c4cccc(N(c5ccc(-c6ccccc6)cc5)c5ccc6c(c5)C(C([2H])([2H])[2H])(C([2H])([2H])[2H])c5ccccc5-6)c4)cc23)c([2H])c1[2H] ; [2H]c1c([2H])c([2H])c(-c2c([2H])c([2H])c(N(c3cccc(-c4cc(-c5ccccc5)c5oc6c(-c7ccccc7)cccc6c5c4)c3)c3cccc4c3-c3ccccc3C43c4ccccc4-c4ccccc43)c([2H])c2[2H])c([2H])c1[2H]]

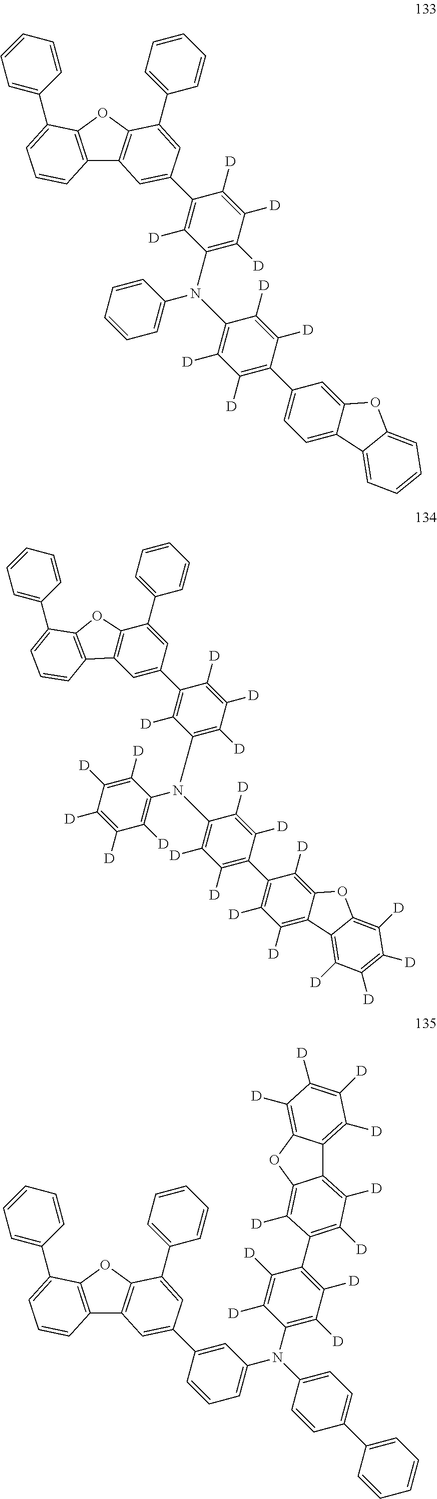

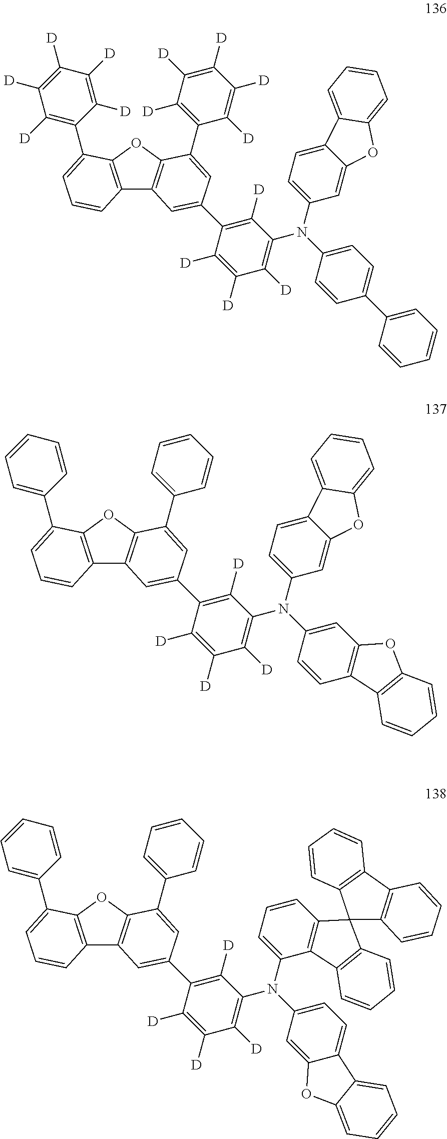 [SMILES: [2H]c1c([2H])c([2H])c(-c2cccc3c2oc2c(-c4c([2H])c([2H])c([2H])c([2H])c4[2H])cc(-c4c([2H])c([2H])c([2H])c(N(c5ccc(-c6ccccc6)cc5)c5ccc6c(c5)oc5ccccc56)c4[2H])cc23)c([2H])c1[2H] ; [2H]c1c([2H])c(-c2cc(-c3ccccc3)c3oc4c(-c5ccccc5)cccc4c3c2)c([2H])c(N(c2ccc3c(c2)oc2ccccc23)c2ccc3c(c2)oc2ccccc23)c1[2H] ; [2H]c1c([2H])c(-c2cc(-c3ccccc3)c3oc4c(-c5ccccc5)cccc4c3c2)c([2H])c(N(c2ccc3c(c2)oc2ccccc23)c2cccc3c2-c2ccccc2C32c3ccccc3-c3ccccc32)c1[2H]]

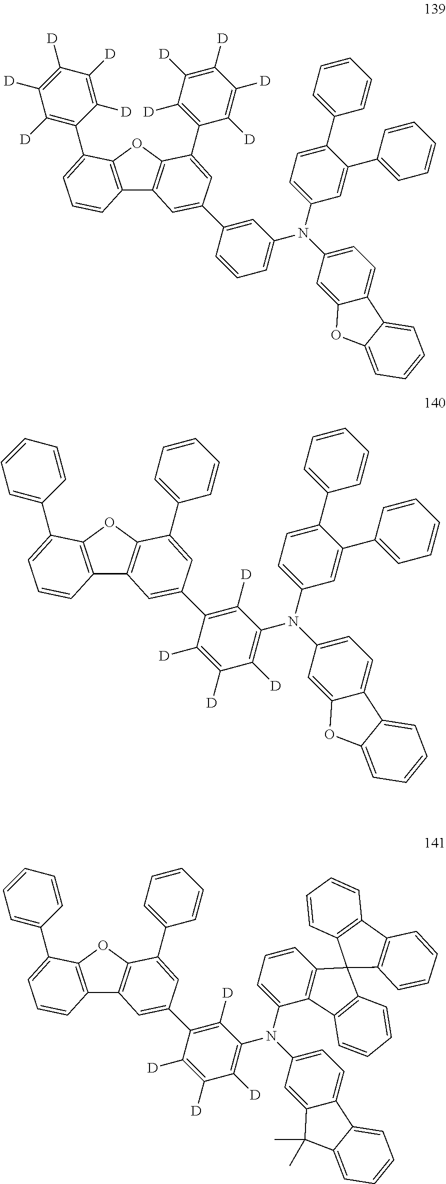 [SMILES: [2H]c1c([2H])c([2H])c(-c2cccc3c2oc2c(-c4c([2H])c([2H])c([2H])c([2H])c4[2H])cc(-c4cccc(N(c5ccc(-c6ccccc6)c(-c6ccccc6)c5)c5ccc6c(c5)oc5ccccc56)c4)cc23)c([2H])c1[2H] ; [2H]c1c([2H])c(-c2cc(-c3ccccc3)c3oc4c(-c5ccccc5)cccc4c3c2)c([2H])c(N(c2ccc(-c3ccccc3)c(-c3ccccc3)c2)c2ccc3c(c2)oc2ccccc23)c1[2H] ; [2H]c1c([2H])c(-c2cc(-c3ccccc3)c3oc4c(-c5ccccc5)cccc4c3c2)c([2H])c(N(c2ccc3c(c2)C(C)(C)c2ccccc2-3)c2cccc3c2-c2ccccc2C32c3ccccc3-c3ccccc32)c1[2H]]

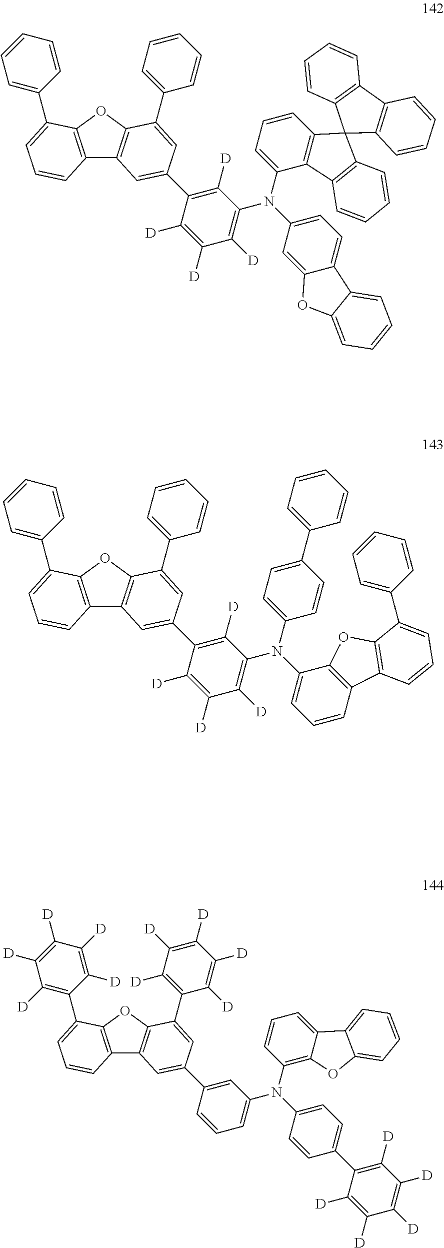 [SMILES: [2H]c1c([2H])c(-c2cc(-c3ccccc3)c3oc4c(-c5ccccc5)cccc4c3c2)c([2H])c(N(c2ccc3c(c2)oc2ccccc23)c2cccc3c2-c2ccccc2C32c3ccccc3-c3ccccc32)c1[2H] ; [2H]c1c([2H])c(-c2cc(-c3ccccc3)c3oc4c(-c5ccccc5)cccc4c3c2)c([2H])c(N(c2ccc(-c3ccccc3)cc2)c2cccc3c2oc2c(-c4ccccc4)cccc23)c1[2H] ; [2H]c1c([2H])c([2H])c(-c2ccc(N(c3cccc(-c4cc(-c5c([2H])c([2H])c([2H])c([2H])c5[2H])c5oc6c(-c7c([2H])c([2H])c([2H])c([2H])c7[2H])cccc6c5c4)c3)c3cccc4c3oc3ccccc34)cc2)c([2H])c1[2H]]

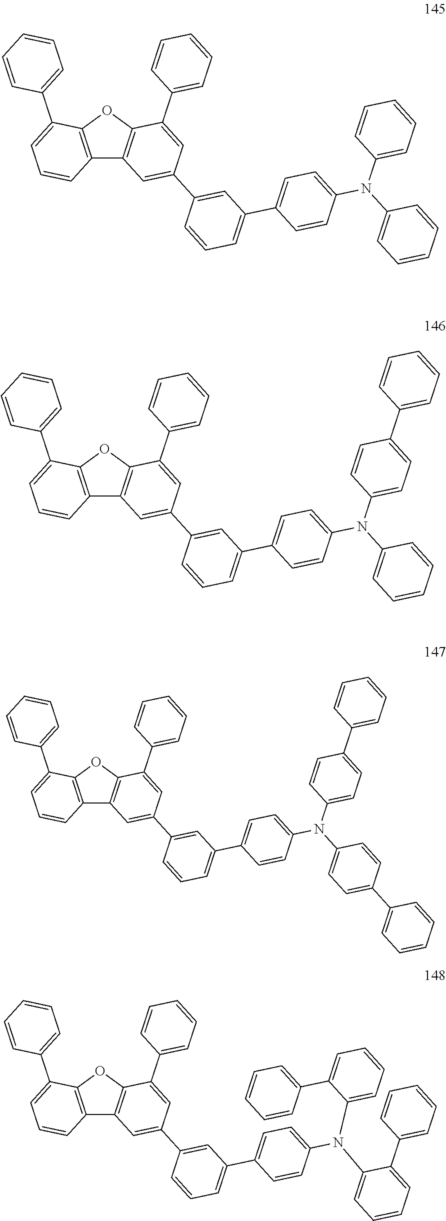 [SMILES: c1ccc(-c2cccc3c2oc2c(-c4ccccc4)cc(-c4cccc(-c5ccc(N(c6ccccc6)c6ccccc6)cc5)c4)cc23)cc1 ; c1ccc(-c2ccc(N(c3ccccc3)c3ccc(-c4cccc(-c5cc(-c6ccccc6)c6oc7c(-c8ccccc8)cccc7c6c5)c4)cc3)cc2)cc1 ; c1ccc(-c2ccc(N(c3ccc(-c4ccccc4)cc3)c3ccc(-c4cccc(-c5cc(-c6ccccc6)c6oc7c(-c8ccccc8)cccc7c6c5)c4)cc3)cc2)cc1 ; c1ccc(-c2ccccc2N(c2ccc(-c3cccc(-c4cc(-c5ccccc5)c5oc6c(-c7ccccc7)cccc6c5c4)c3)cc2)c2ccccc2-c2ccccc2)cc1]

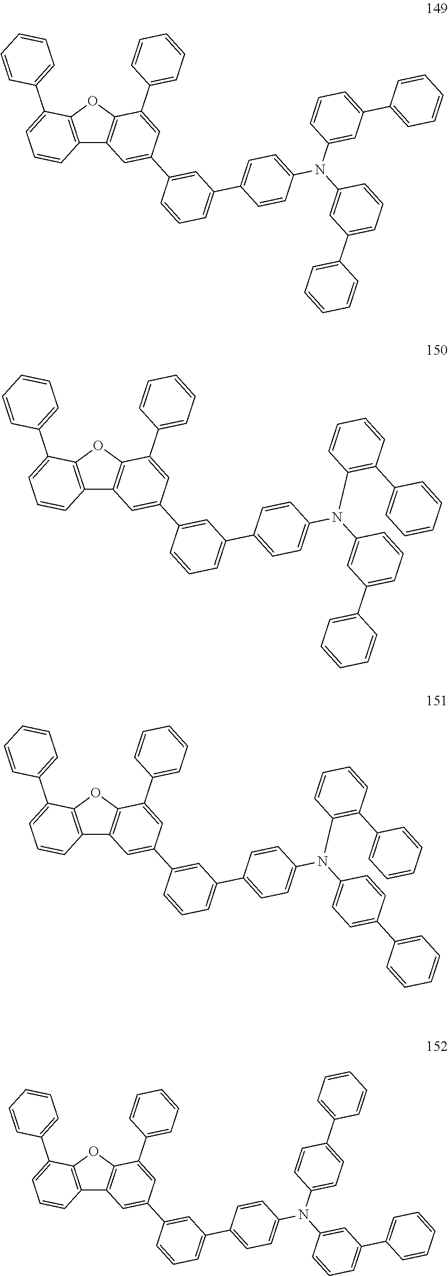 [SMILES: c1ccc(-c2cccc(N(c3ccc(-c4cccc(-c5cc(-c6ccccc6)c6oc7c(-c8ccccc8)cccc7c6c5)c4)cc3)c3cccc(-c4ccccc4)c3)c2)cc1 ; c1ccc(-c2cccc(N(c3ccc(-c4cccc(-c5cc(-c6ccccc6)c6oc7c(-c8ccccc8)cccc7c6c5)c4)cc3)c3ccccc3-c3ccccc3)c2)cc1 ; c1ccc(-c2ccc(N(c3ccc(-c4cccc(-c5cc(-c6ccccc6)c6oc7c(-c8ccccc8)cccc7c6c5)c4)cc3)c3ccccc3-c3ccccc3)cc2)cc1 ; c1ccc(-c2ccc(N(c3ccc(-c4cccc(-c5cc(-c6ccccc6)c6oc7c(-c8ccccc8)cccc7c6c5)c4)cc3)c3cccc(-c4ccccc4)c3)cc2)cc1]

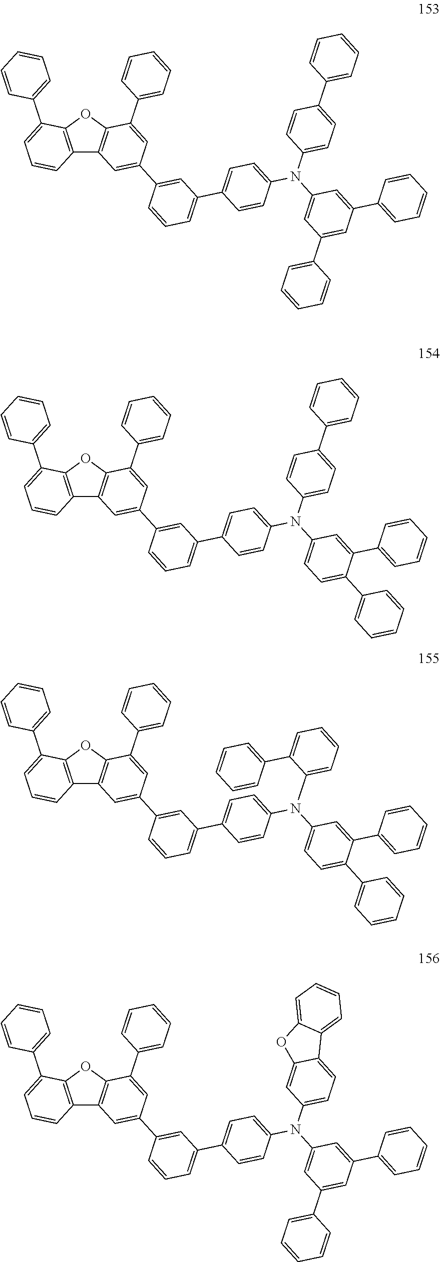 [SMILES: c1ccc(-c2ccc(N(c3ccc(-c4cccc(-c5cc(-c6ccccc6)c6oc7c(-c8ccccc8)cccc7c6c5)c4)cc3)c3cc(-c4ccccc4)cc(-c4ccccc4)c3)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc(-c4ccccc4)c(-c4ccccc4)c3)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccccc3-c3ccccc3)cc2-c2ccccc2)cc1 ; c1ccc(-c2cc(-c3ccccc3)cc(N(c3ccc(-c4cccc(-c5cc(-c6ccccc6)c6oc7c(-c8ccccc8)cccc7c6c5)c4)cc3)c3ccc4c(c3)oc3ccccc34)c2)cc1]

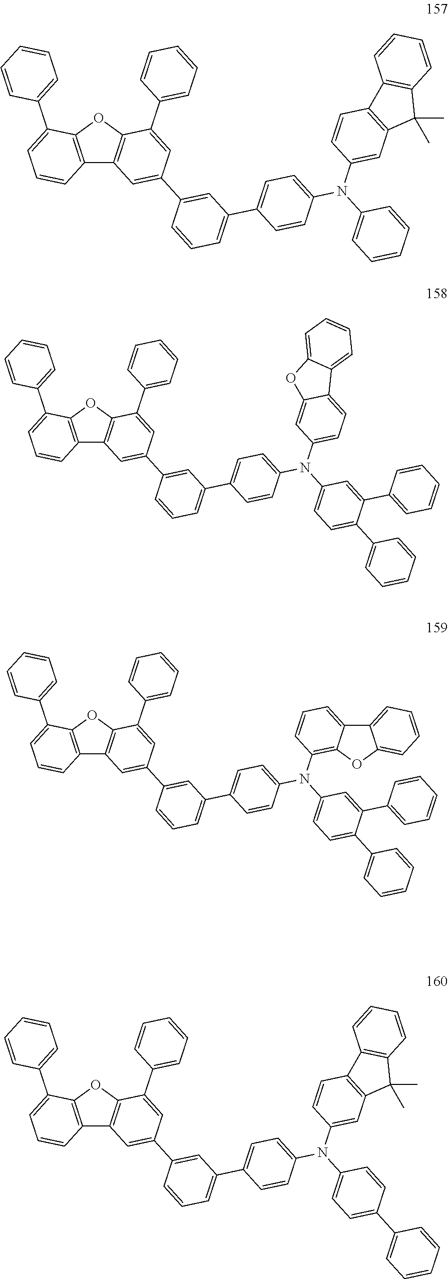 [SMILES: CC1(C)c2ccccc2-c2ccc(N(c3ccccc3)c3ccc(-c4cccc(-c5cc(-c6ccccc6)c6oc7c(-c8ccccc8)cccc7c6c5)c4)cc3)cc21 ; c1ccc(-c2ccc(N(c3ccc(-c4cccc(-c5cc(-c6ccccc6)c6oc7c(-c8ccccc8)cccc7c6c5)c4)cc3)c3ccc4c(c3)oc3ccccc34)cc2-c2ccccc2)cc1 ; c1ccc(-c2ccc(N(c3ccc(-c4cccc(-c5cc(-c6ccccc6)c6oc7c(-c8ccccc8)cccc7c6c5)c4)cc3)c3cccc4c3oc3ccccc34)cc2-c2ccccc2)cc1 ; CC1(C)c2ccccc2-c2ccc(N(c3ccc(-c4ccccc4)cc3)c3ccc(-c4cccc(-c5cc(-c6ccccc6)c6oc7c(-c8ccccc8)cccc7c6c5)c4)cc3)cc21]

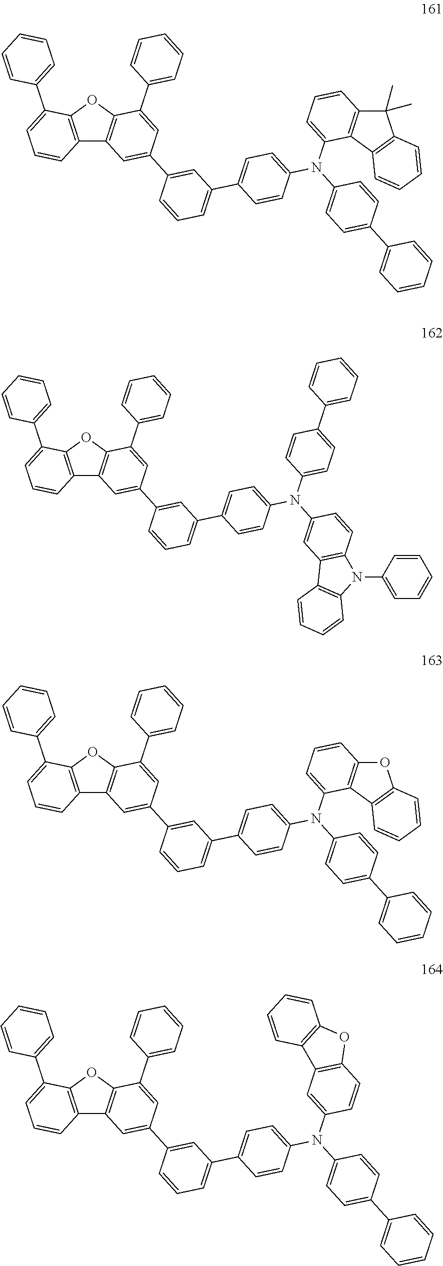 [SMILES: CC1(C)c2ccccc2-c2c(N(c3ccc(-c4ccccc4)cc3)c3ccc(-c4cccc(-c5cc(-c6ccccc6)c6oc7c(-c8ccccc8)cccc7c6c5)c4)cc3)cccc21 ; c1ccc(-c2ccc(N(c3ccc(-c4cccc(-c5cc(-c6ccccc6)c6oc7c(-c8ccccc8)cccc7c6c5)c4)cc3)c3ccc4c(c3)c3ccccc3n4-c3ccccc3)cc2)cc1 ; c1ccc(-c2ccc(N(c3ccc(-c4cccc(-c5cc(-c6ccccc6)c6oc7c(-c8ccccc8)cccc7c6c5)c4)cc3)c3cccc4oc5ccccc5c34)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4oc5ccccc5c4c3)cc2)cc1]

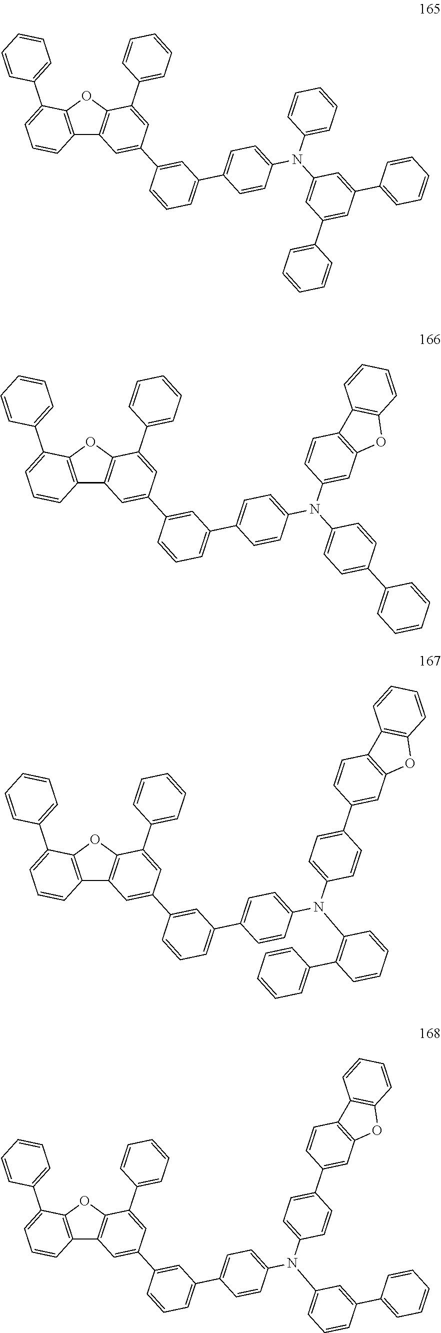 [SMILES: c1ccc(-c2cc(-c3ccccc3)cc(N(c3ccccc3)c3ccc(-c4cccc(-c5cc(-c6ccccc6)c6oc7c(-c8ccccc8)cccc7c6c5)c4)cc3)c2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)oc3ccccc34)cc2)cc1 ; c1ccc(-c2ccccc2N(c2ccc(-c3cccc(-c4cc(-c5ccccc5)c5oc6c(-c7ccccc7)cccc6c5c4)c3)cc2)c2ccc(-c3ccc4c(c3)oc3ccccc34)cc2)cc1 ; c1ccc(-c2cccc(N(c3ccc(-c4cccc(-c5cc(-c6ccccc6)c6oc7c(-c8ccccc8)cccc7c6c5)c4)cc3)c3ccc(-c4ccc5c(c4)oc4ccccc45)cc3)c2)cc1]

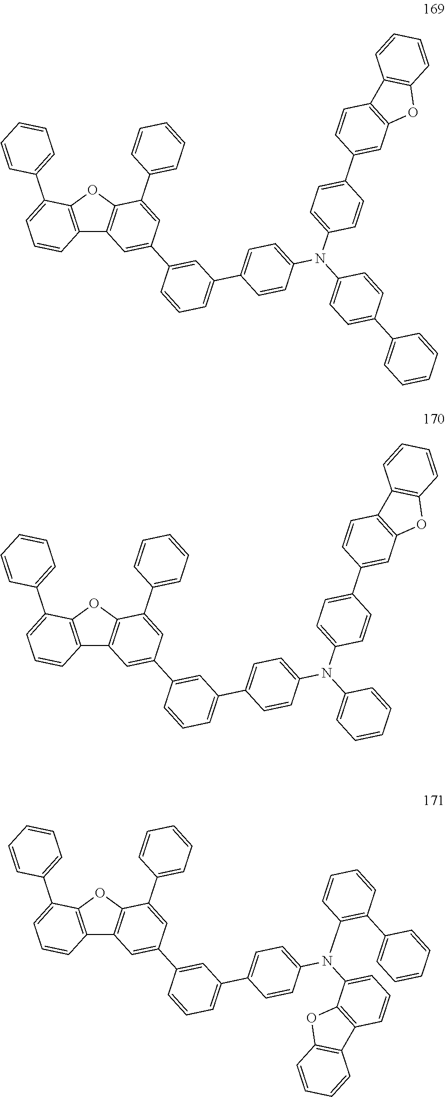 [SMILES: c1ccc(-c2ccc(N(c3ccc(-c4cccc(-c5cc(-c6ccccc6)c6oc7c(-c8ccccc8)cccc7c6c5)c4)cc3)c3ccc(-c4ccc5c(c4)oc4ccccc45)cc3)cc2)cc1 ; c1ccc(-c2cccc3c2oc2c(-c4ccccc4)cc(-c4cccc(-c5ccc(N(c6ccccc6)c6ccc(-c7ccc8c(c7)oc7ccccc78)cc6)cc5)c4)cc23)cc1 ; c1ccc(-c2ccccc2N(c2ccc(-c3cccc(-c4cc(-c5ccccc5)c5oc6c(-c7ccccc7)cccc6c5c4)c3)cc2)c2cccc3c2oc2ccccc23)cc1]

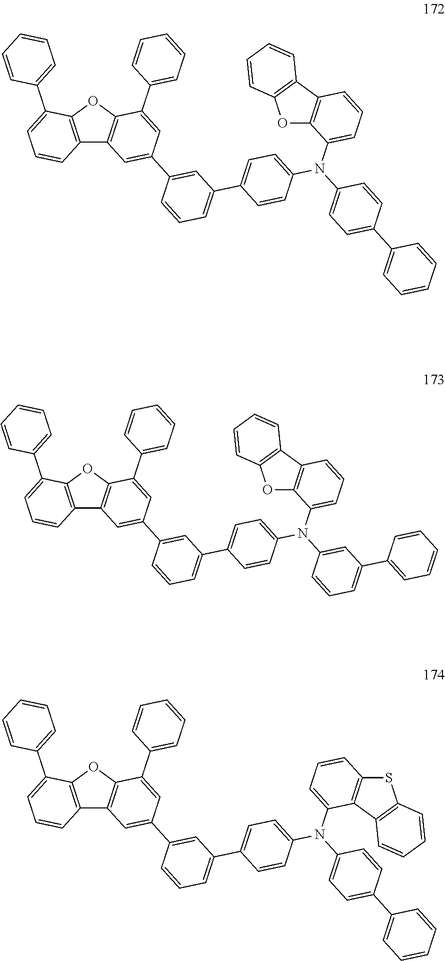 [SMILES: c1ccc(-c2ccc(N(c3ccc(-c4cccc(-c5cc(-c6ccccc6)c6oc7c(-c8ccccc8)cccc7c6c5)c4)cc3)c3cccc4c3oc3ccccc34)cc2)cc1 ; c1ccc(-c2cccc(N(c3ccc(-c4cccc(-c5cc(-c6ccccc6)c6oc7c(-c8ccccc8)cccc7c6c5)c4)cc3)c3cccc4c3oc3ccccc34)c2)cc1 ; c1ccc(-c2ccc(N(c3ccc(-c4cccc(-c5cc(-c6ccccc6)c6oc7c(-c8ccccc8)cccc7c6c5)c4)cc3)c3cccc4sc5ccccc5c34)cc2)cc1]

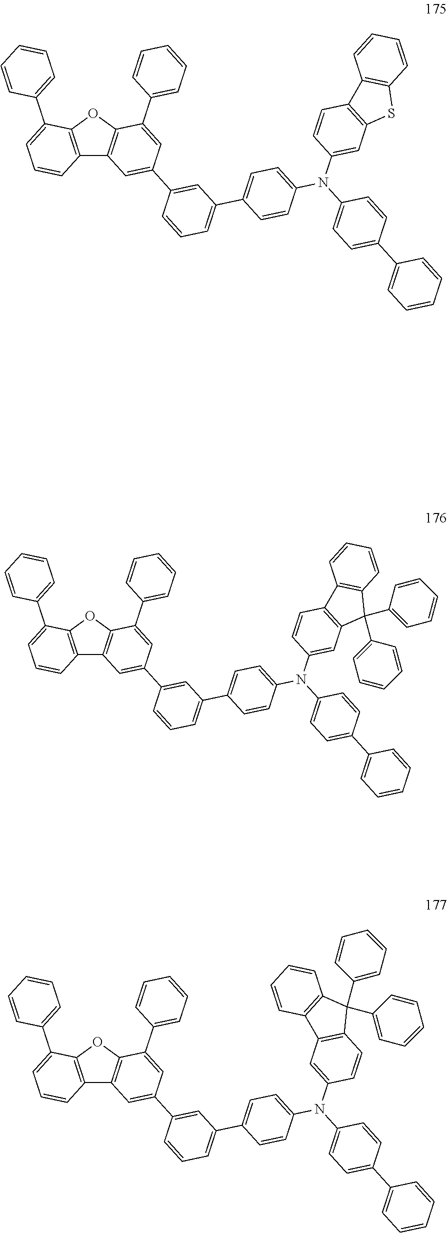 [SMILES: c1ccc(-c2ccc(N(c3ccc(-c4cccc(-c5cc(-c6ccccc6)c6oc7c(-c8ccccc8)cccc7c6c5)c4)cc3)c3ccc4c(c3)sc3ccccc34)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)-c3ccccc3C4(c3ccccc3)c3ccccc3)cc2)cc1]

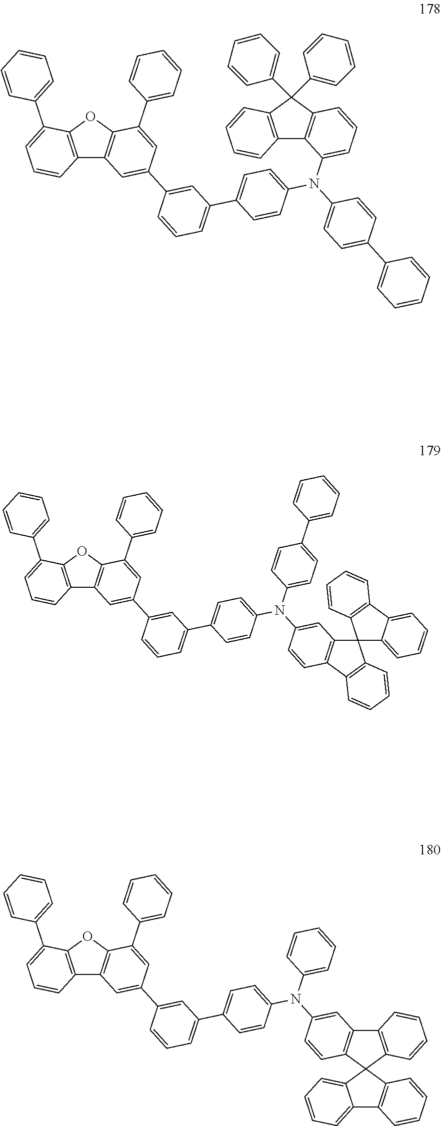 [SMILES: c1ccc(-c2ccc(N(c3ccc(-c4cccc(-c5cc(-c6ccccc6)c6oc7c(-c8ccccc8)cccc7c6c5)c4)cc3)c3cccc4c3-c3ccccc3C4(c3ccccc3)c3ccccc3)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)C3(c5ccccc5-c5ccccc53)c3ccccc3-4)cc2)cc1 ; c1ccc(-c2cccc3c2oc2c(-c4ccccc4)cc(-c4cccc(-c5ccc(N(c6ccccc6)c6ccc7c(c6)-c6ccccc6C76c7ccccc7-c7ccccc76)cc5)c4)cc23)cc1]

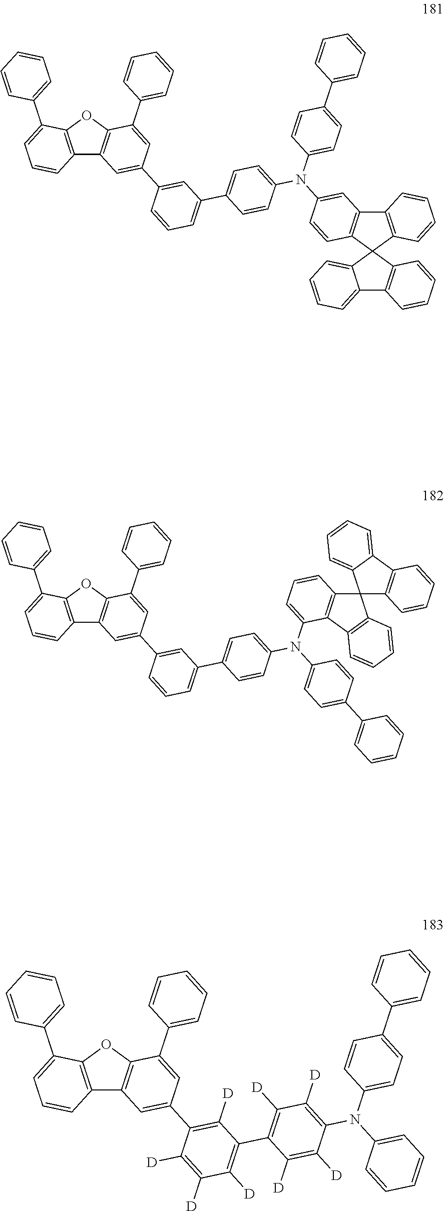 [SMILES: c1ccc(-c2ccc(N(c3ccc(-c4cccc(-c5cc(-c6ccccc6)c6oc7c(-c8ccccc8)cccc7c6c5)c4)cc3)c3ccc4c(c3)-c3ccccc3C43c4ccccc4-c4ccccc43)cc2)cc1 ; c1ccc(-c2ccc(N(c3ccc(-c4cccc(-c5cc(-c6ccccc6)c6oc7c(-c8ccccc8)cccc7c6c5)c4)cc3)c3cccc4c3-c3ccccc3C43c4ccccc4-c4ccccc43)cc2)cc1 ; [2H]c1c([2H])c(-c2cc(-c3ccccc3)c3oc4c(-c5ccccc5)cccc4c3c2)c([2H])c(-c2c([2H])c([2H])c(N(c3ccccc3)c3ccc(-c4ccccc4)cc3)c([2H])c2[2H])c1[2H]]

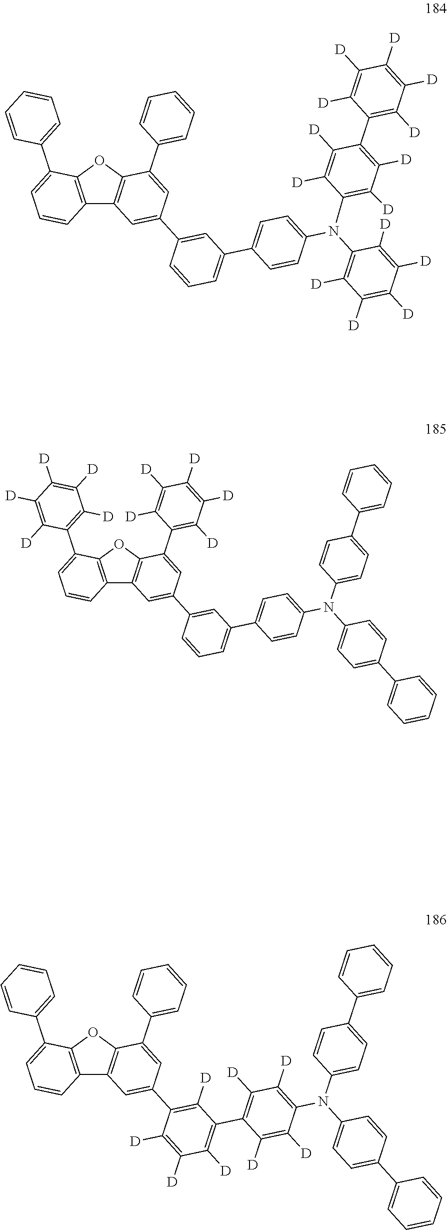 [SMILES: [2H]c1c([2H])c([2H])c(-c2c([2H])c([2H])c(N(c3ccc(-c4cccc(-c5cc(-c6ccccc6)c6oc7c(-c8ccccc8)cccc7c6c5)c4)cc3)c3c([2H])c([2H])c([2H])c([2H])c3[2H])c([2H])c2[2H])c([2H])c1[2H] ; [2H]c1c([2H])c([2H])c(-c2cccc3c2oc2c(-c4c([2H])c([2H])c([2H])c([2H])c4[2H])cc(-c4cccc(-c5ccc(N(c6ccc(-c7ccccc7)cc6)c6ccc(-c7ccccc7)cc6)cc5)c4)cc23)c([2H])c1[2H] ; [2H]c1c([2H])c(-c2cc(-c3ccccc3)c3oc4c(-c5ccccc5)cccc4c3c2)c([2H])c(-c2c([2H])c([2H])c(N(c3ccc(-c4ccccc4)cc3)c3ccc(-c4ccccc4)cc3)c([2H])c2[2H])c1[2H]]

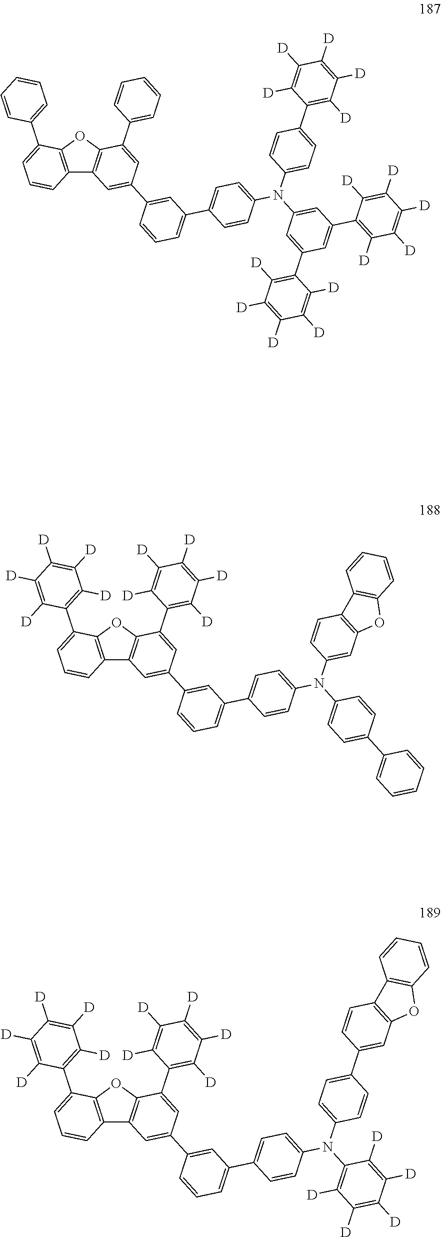

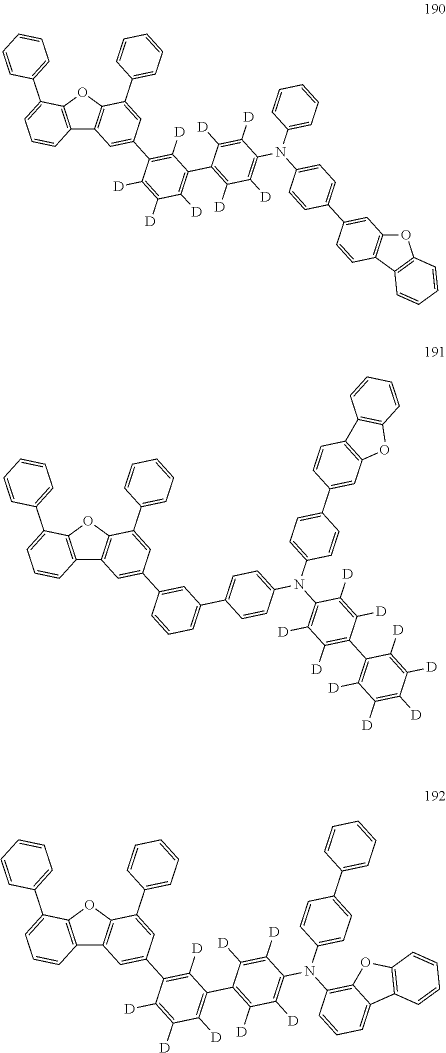 [SMILES: [2H]c1c([2H])c(-c2cc(-c3ccccc3)c3oc4c(-c5ccccc5)cccc4c3c2)c([2H])c(-c2c([2H])c([2H])c(N(c3ccccc3)c3ccc(-c4ccc5c(c4)oc4ccccc45)cc3)c([2H])c2[2H])c1[2H] ; [2H]c1c([2H])c([2H])c(-c2c([2H])c([2H])c(N(c3ccc(-c4cccc(-c5cc(-c6ccccc6)c6oc7c(-c8ccccc8)cccc7c6c5)c4)cc3)c3ccc(-c4ccc5c(c4)oc4ccccc45)cc3)c([2H])c2[2H])c([2H])c1[2H] ; [2H]c1c([2H])c(-c2cc(-c3ccccc3)c3oc4c(-c5ccccc5)cccc4c3c2)c([2H])c(-c2c([2H])c([2H])c(N(c3ccc(-c4ccccc4)cc3)c3cccc4c3oc3ccccc34)c([2H])c2[2H])c1[2H]]

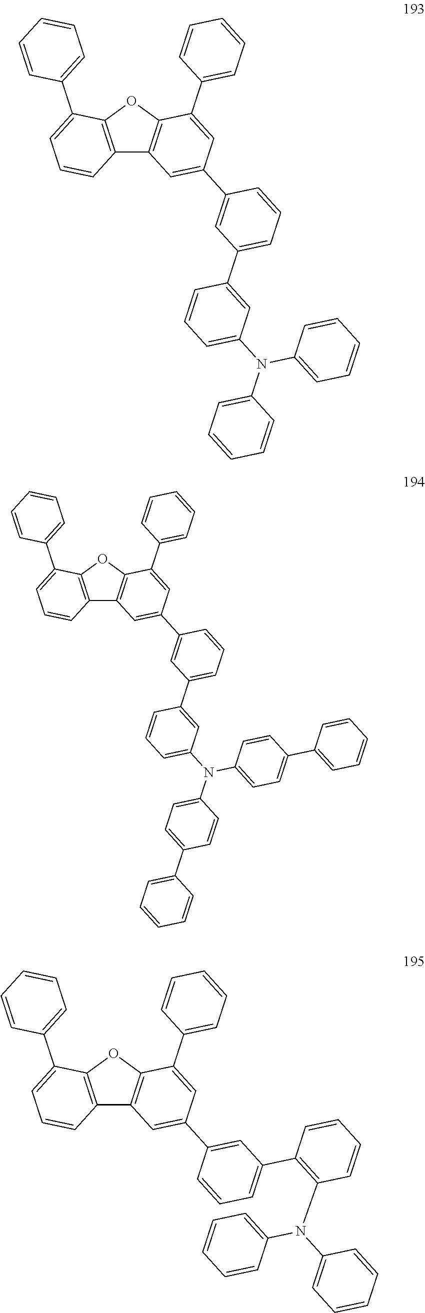 [SMILES: c1ccc(-c2cccc3c2oc2c(-c4ccccc4)cc(-c4cccc(-c5cccc(N(c6ccccc6)c6ccccc6)c5)c4)cc23)cc1 ; c1ccc(-c2ccc(N(c3ccc(-c4ccccc4)cc3)c3cccc(-c4cccc(-c5cc(-c6ccccc6)c6oc7c(-c8ccccc8)cccc7c6c5)c4)c3)cc2)cc1 ; c1ccc(-c2cccc3c2oc2c(-c4ccccc4)cc(-c4cccc(-c5ccccc5N(c5ccccc5)c5ccccc5)c4)cc23)cc1]

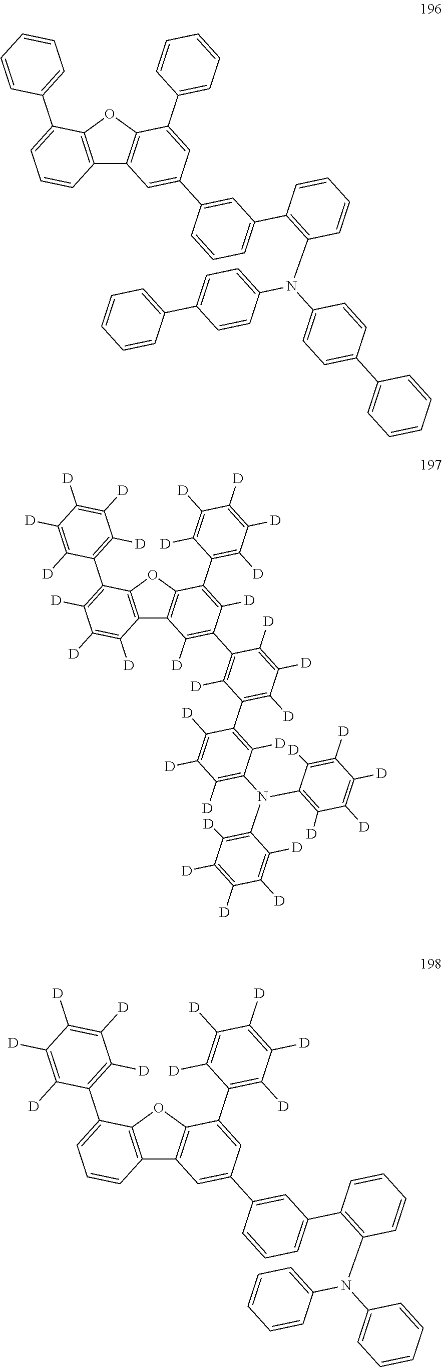

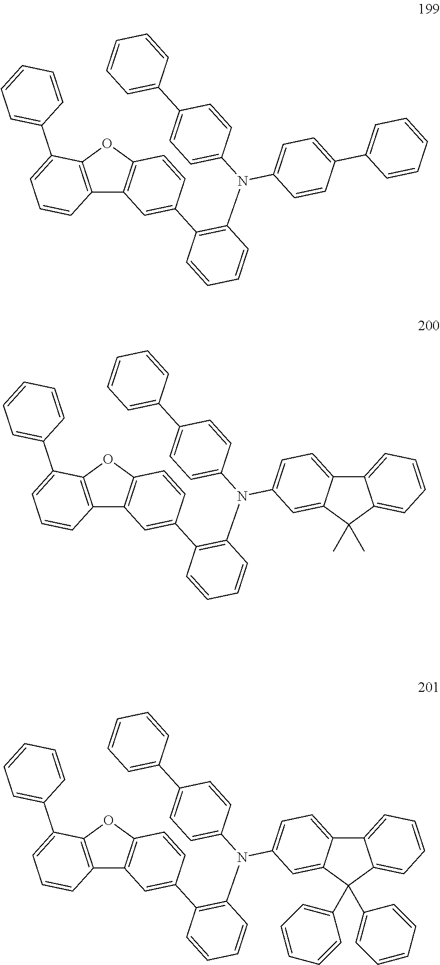 [SMILES: c1ccc(-c2ccc(N(c3ccc(-c4ccccc4)cc3)c3ccccc3-c3ccc4oc5c(-c6ccccc6)cccc5c4c3)cc2)cc1 ; CC1(C)c2ccccc2-c2ccc(N(c3ccc(-c4ccccc4)cc3)c3ccccc3-c3ccc4oc5c(-c6ccccc6)cccc5c4c3)cc21 ; c1ccc(-c2ccc(N(c3ccc4c(c3)C(c3ccccc3)(c3ccccc3)c3ccccc3-4)c3ccccc3-c3ccc4oc5c(-c6ccccc6)cccc5c4c3)cc2)cc1]

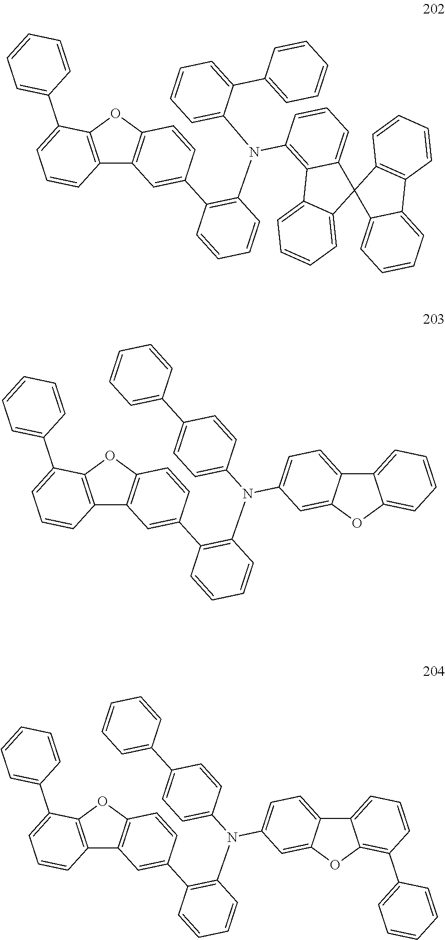 [SMILES: c1ccc(-c2ccccc2N(c2ccccc2-c2ccc3oc4c(-c5ccccc5)cccc4c3c2)c2cccc3c2-c2ccccc2C32c3ccccc3-c3ccccc32)cc1 ; c1ccc(-c2ccc(N(c3ccc4c(c3)oc3ccccc34)c3ccccc3-c3ccc4oc5c(-c6ccccc6)cccc5c4c3)cc2)cc1 ; c1ccc(-c2ccc(N(c3ccc4c(c3)oc3c(-c5ccccc5)cccc34)c3ccccc3-c3ccc4oc5c(-c6ccccc6)cccc5c4c3)cc2)cc1]

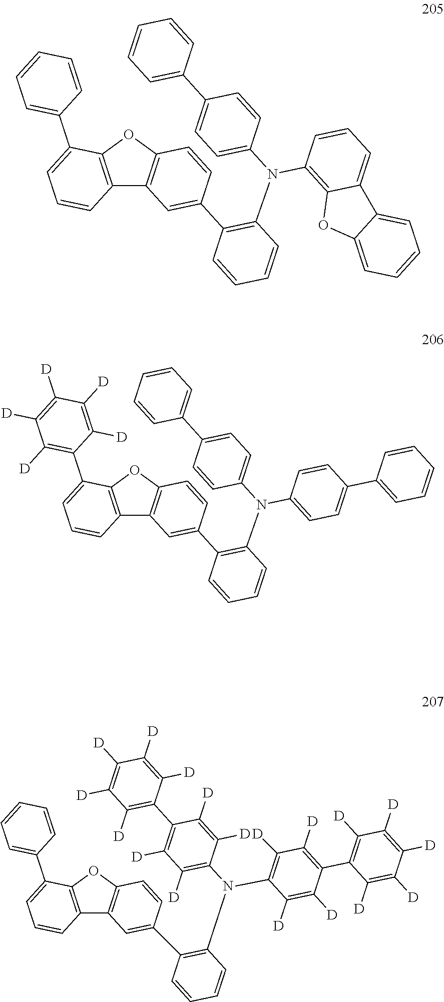 [SMILES: c1ccc(-c2ccc(N(c3ccccc3-c3ccc4oc5c(-c6ccccc6)cccc5c4c3)c3cccc4c3oc3ccccc34)cc2)cc1 ; [2H]c1c([2H])c([2H])c(-c2cccc3c2oc2ccc(-c4ccccc4N(c4ccc(-c5ccccc5)cc4)c4ccc(-c5ccccc5)cc4)cc23)c([2H])c1[2H] ; [2H]c1c([2H])c([2H])c(-c2c([2H])c([2H])c(N(c3ccccc3-c3ccc4oc5c(-c6ccccc6)cccc5c4c3)c3c([2H])c([2H])c(-c4c([2H])c([2H])c([2H])c([2H])c4[2H])c([2H])c3[2H])c([2H])c2[2H])c([2H])c1[2H]]

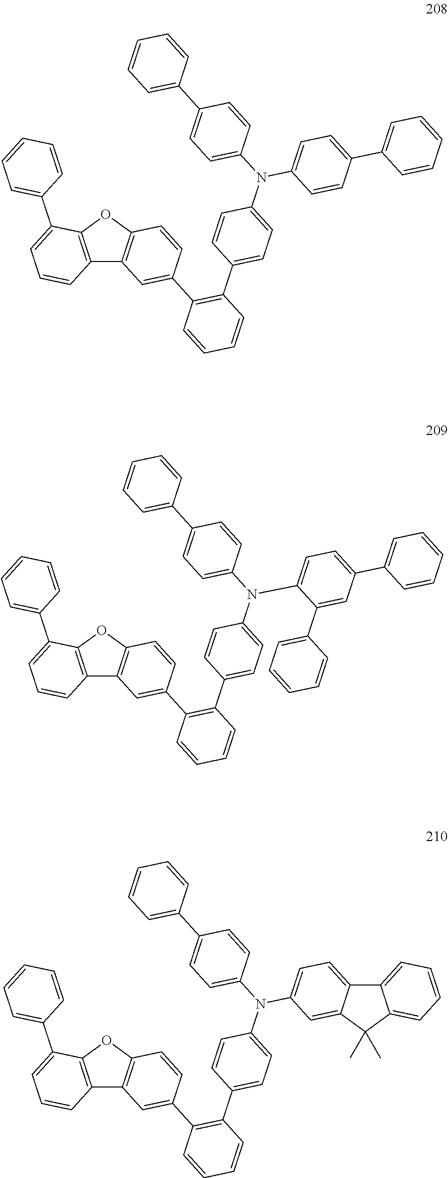 [SMILES: c1ccc(-c2ccc(N(c3ccc(-c4ccccc4)cc3)c3ccc(-c4ccccc4-c4ccc5oc6c(-c7ccccc7)cccc6c5c4)cc3)cc2)cc1 ; c1ccc(-c2ccc(N(c3ccc(-c4ccccc4-c4ccc5oc6c(-c7ccccc7)cccc6c5c4)cc3)c3ccc(-c4ccccc4)cc3-c3ccccc3)cc2)cc1 ; CC1(C)c2ccccc2-c2ccc(N(c3ccc(-c4ccccc4)cc3)c3ccc(-c4ccccc4-c4ccc5oc6c(-c7ccccc7)cccc6c5c4)cc3)cc21]

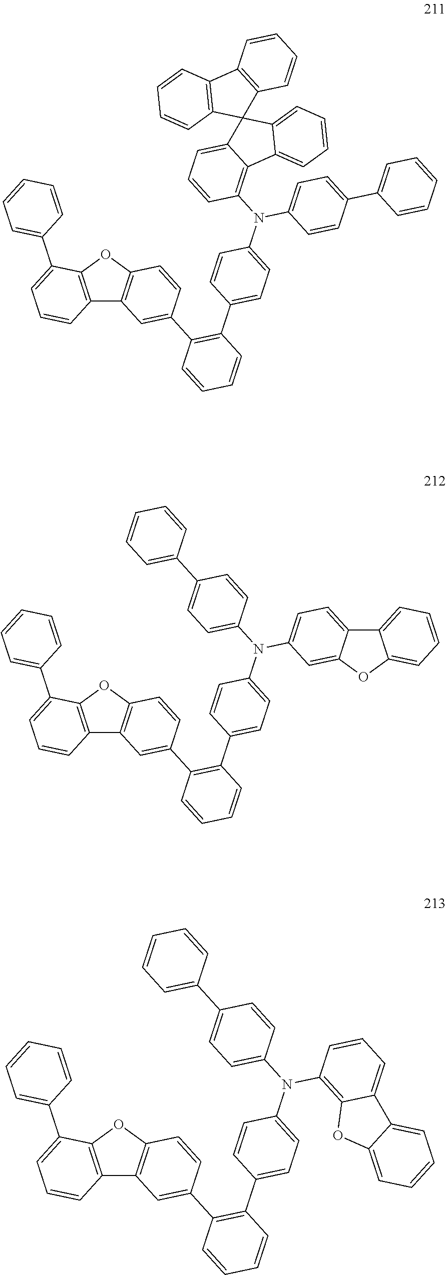 [SMILES: c1ccc(-c2ccc(N(c3ccc(-c4ccccc4-c4ccc5oc6c(-c7ccccc7)cccc6c5c4)cc3)c3cccc4c3-c3ccccc3C43c4ccccc4-c4ccccc43)cc2)cc1 ; c1ccc(-c2ccc(N(c3ccc(-c4ccccc4-c4ccc5oc6c(-c7ccccc7)cccc6c5c4)cc3)c3ccc4c(c3)oc3ccccc34)cc2)cc1 ; c1ccc(-c2ccc(N(c3ccc(-c4ccccc4-c4ccc5oc6c(-c7ccccc7)cccc6c5c4)cc3)c3cccc4c3oc3ccccc34)cc2)cc1]

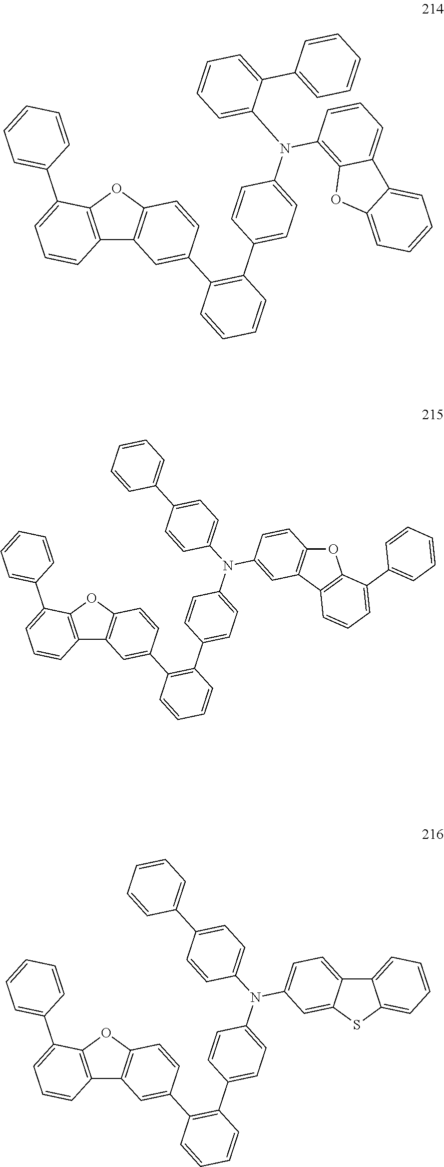 [SMILES: c1ccc(-c2ccccc2N(c2ccc(-c3ccccc3-c3ccc4oc5c(-c6ccccc6)cccc5c4c3)cc2)c2cccc3c2oc2ccccc23)cc1 ; c1ccc(-c2ccc(N(c3ccc(-c4ccccc4-c4ccc5oc6c(-c7ccccc7)cccc6c5c4)cc3)c3ccc4oc5c(-c6ccccc6)cccc5c4c3)cc2)cc1 ; c1ccc(-c2ccc(N(c3ccc(-c4ccccc4-c4ccc5oc6c(-c7ccccc7)cccc6c5c4)cc3)c3ccc4c(c3)sc3ccccc34)cc2)cc1]

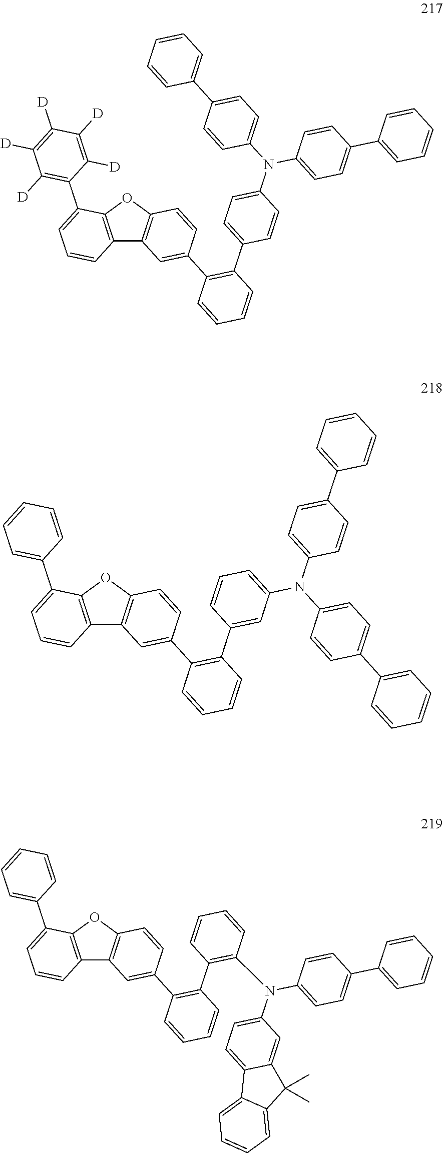 [SMILES: [2H]c1c([2H])c([2H])c(-c2cccc3c2oc2ccc(-c4ccccc4-c4ccc(N(c5ccc(-c6ccccc6)cc5)c5ccc(-c6ccccc6)cc5)cc4)cc23)c([2H])c1[2H] ; c1ccc(-c2ccc(N(c3ccc(-c4ccccc4)cc3)c3cccc(-c4ccccc4-c4ccc5oc6c(-c7ccccc7)cccc6c5c4)c3)cc2)cc1 ; CC1(C)c2ccccc2-c2ccc(N(c3ccc(-c4ccccc4)cc3)c3ccccc3-c3ccccc3-c3ccc4oc5c(-c6ccccc6)cccc5c4c3)cc21]

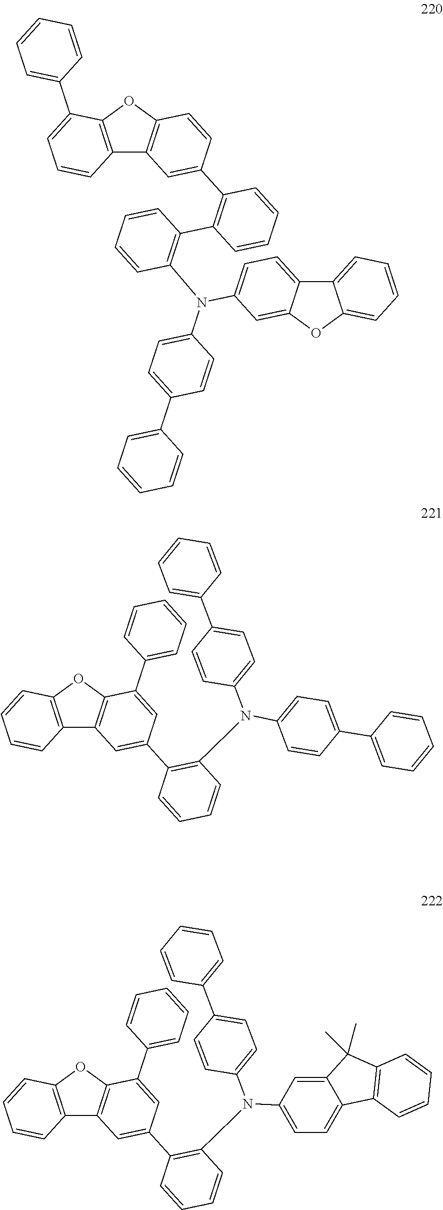 [SMILES: c1ccc(-c2ccc(N(c3ccc4c(c3)oc3ccccc34)c3ccccc3-c3ccccc3-c3ccc4oc5c(-c6ccccc6)cccc5c4c3)cc2)cc1 ; c1ccc(-c2ccc(N(c3ccc(-c4ccccc4)cc3)c3ccccc3-c3cc(-c4ccccc4)c4oc5ccccc5c4c3)cc2)cc1 ; CC1(C)c2ccccc2-c2ccc(N(c3ccc(-c4ccccc4)cc3)c3ccccc3-c3cc(-c4ccccc4)c4oc5ccccc5c4c3)cc21]

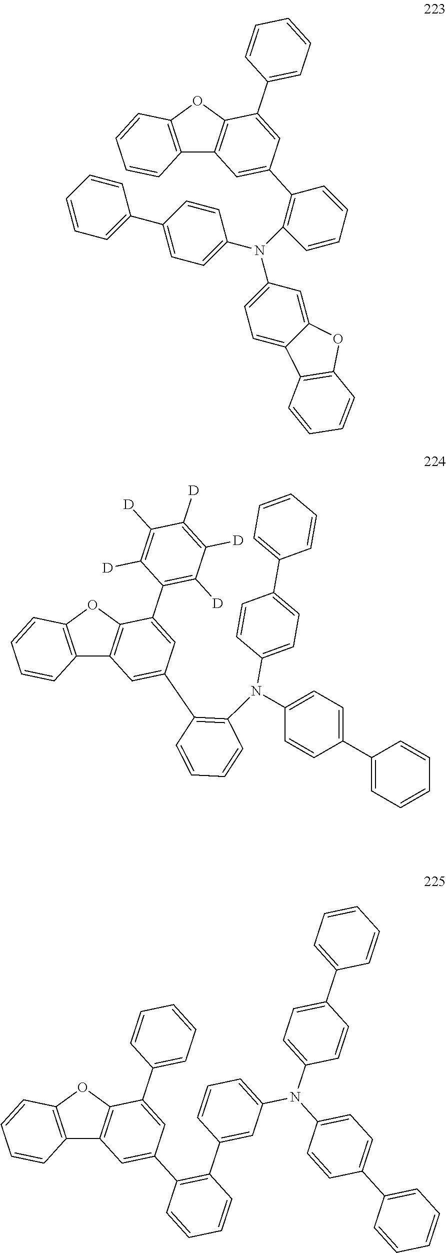 [SMILES: c1ccc(-c2ccc(N(c3ccc4c(c3)oc3ccccc34)c3ccccc3-c3cc(-c4ccccc4)c4oc5ccccc5c4c3)cc2)cc1 ; [2H]c1c([2H])c([2H])c(-c2cc(-c3ccccc3N(c3ccc(-c4ccccc4)cc3)c3ccc(-c4ccccc4)cc3)cc3c2oc2ccccc23)c([2H])c1[2H] ; c1ccc(-c2ccc(N(c3ccc(-c4ccccc4)cc3)c3cccc(-c4ccccc4-c4cc(-c5ccccc5)c5oc6ccccc6c5c4)c3)cc2)cc1]

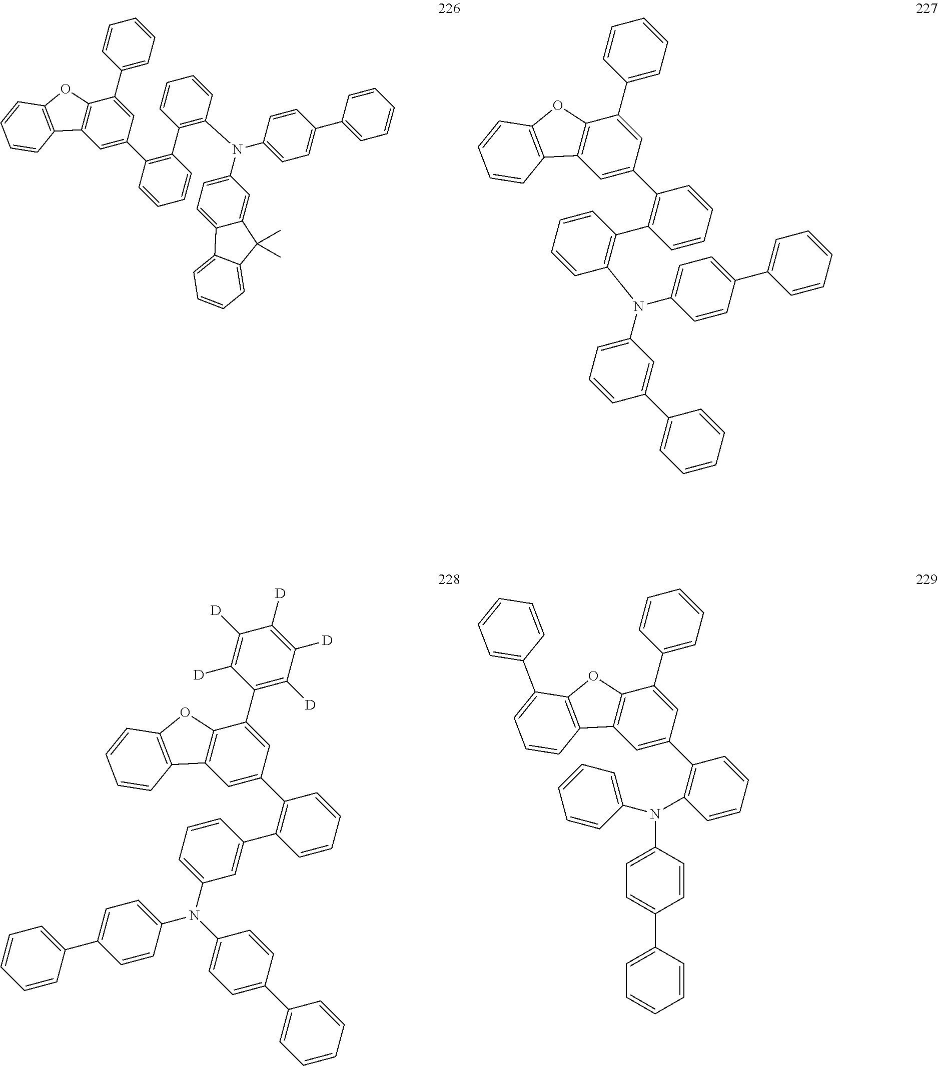 [SMILES: CC1(C)c2ccccc2-c2ccc(N(c3ccc(-c4ccccc4)cc3)c3ccccc3-c3ccccc3-c3cc(-c4ccccc4)c4oc5ccccc5c4c3)cc21 ; c1ccc(-c2ccc(N(c3cccc(-c4ccccc4)c3)c3ccccc3-c3ccccc3-c3cc(-c4ccccc4)c4oc5ccccc5c4c3)cc2)cc1 ; [2H]c1c([2H])c([2H])c(-c2cc(-c3ccccc3-c3cccc(N(c4ccc(-c5ccccc5)cc4)c4ccc(-c5ccccc5)cc4)c3)cc3c2oc2ccccc23)c([2H])c1[2H] ; c1ccc(-c2ccc(N(c3ccccc3)c3ccccc3-c3cc(-c4ccccc4)c4oc5c(-c6ccccc6)cccc5c4c3)cc2)cc1]

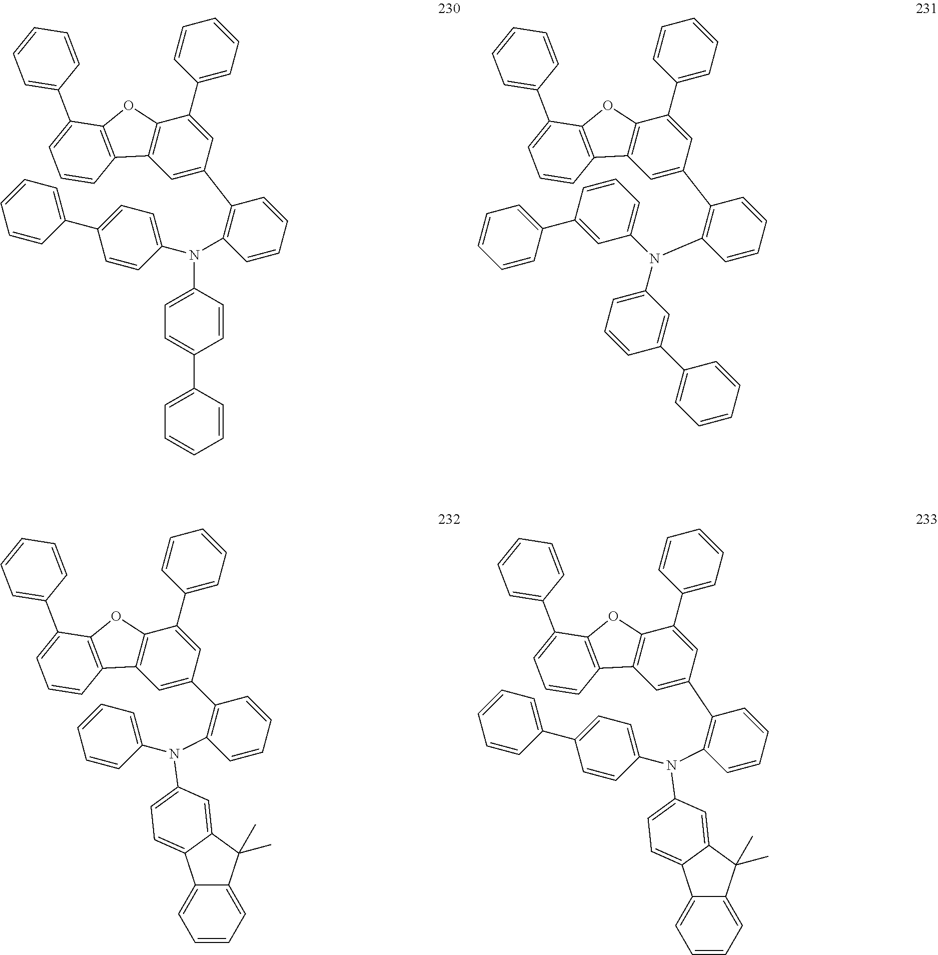 [SMILES: c1ccc(-c2ccc(N(c3ccc(-c4ccccc4)cc3)c3ccccc3-c3cc(-c4ccccc4)c4oc5c(-c6ccccc6)cccc5c4c3)cc2)cc1 ; c1ccc(-c2cccc(N(c3cccc(-c4ccccc4)c3)c3ccccc3-c3cc(-c4ccccc4)c4oc5c(-c6ccccc6)cccc5c4c3)c2)cc1 ; CC1(C)c2ccccc2-c2ccc(N(c3ccccc3)c3ccccc3-c3cc(-c4ccccc4)c4oc5c(-c6ccccc6)cccc5c4c3)cc21 ; CC1(C)c2ccccc2-c2ccc(N(c3ccc(-c4ccccc4)cc3)c3ccccc3-c3cc(-c4ccccc4)c4oc5c(-c6ccccc6)cccc5c4c3)cc21]

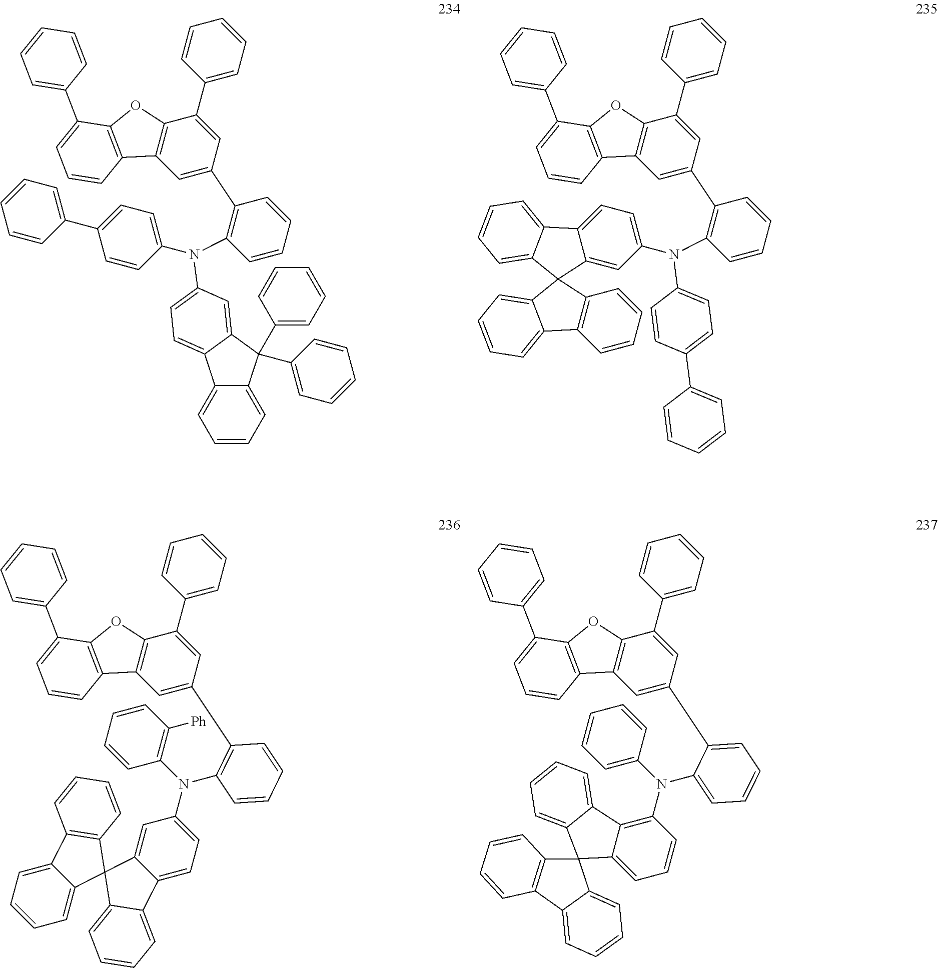 [SMILES: c1ccc(-c2ccc(N(c3ccc4c(c3)C(c3ccccc3)(c3ccccc3)c3ccccc3-4)c3ccccc3-c3cc(-c4ccccc4)c4oc5c(-c6ccccc6)cccc5c4c3)cc2)cc1 ; c1ccc(-c2ccc(N(c3ccc4c(c3)C3(c5ccccc5-c5ccccc53)c3ccccc3-4)c3ccccc3-c3cc(-c4ccccc4)c4oc5c(-c6ccccc6)cccc5c4c3)cc2)cc1 ; c1ccc(-c2ccccc2N(c2ccc3c(c2)C2(c4ccccc4-c4ccccc42)c2ccccc2-3)c2ccccc2-c2cc(-c3ccccc3)c3oc4c(-c5ccccc5)cccc4c3c2)cc1 ; c1ccc(-c2cccc3c2oc2c(-c4ccccc4)cc(-c4ccccc4N(c4ccccc4)c4cccc5c4-c4ccccc4C54c5ccccc5-c5ccccc54)cc23)cc1]

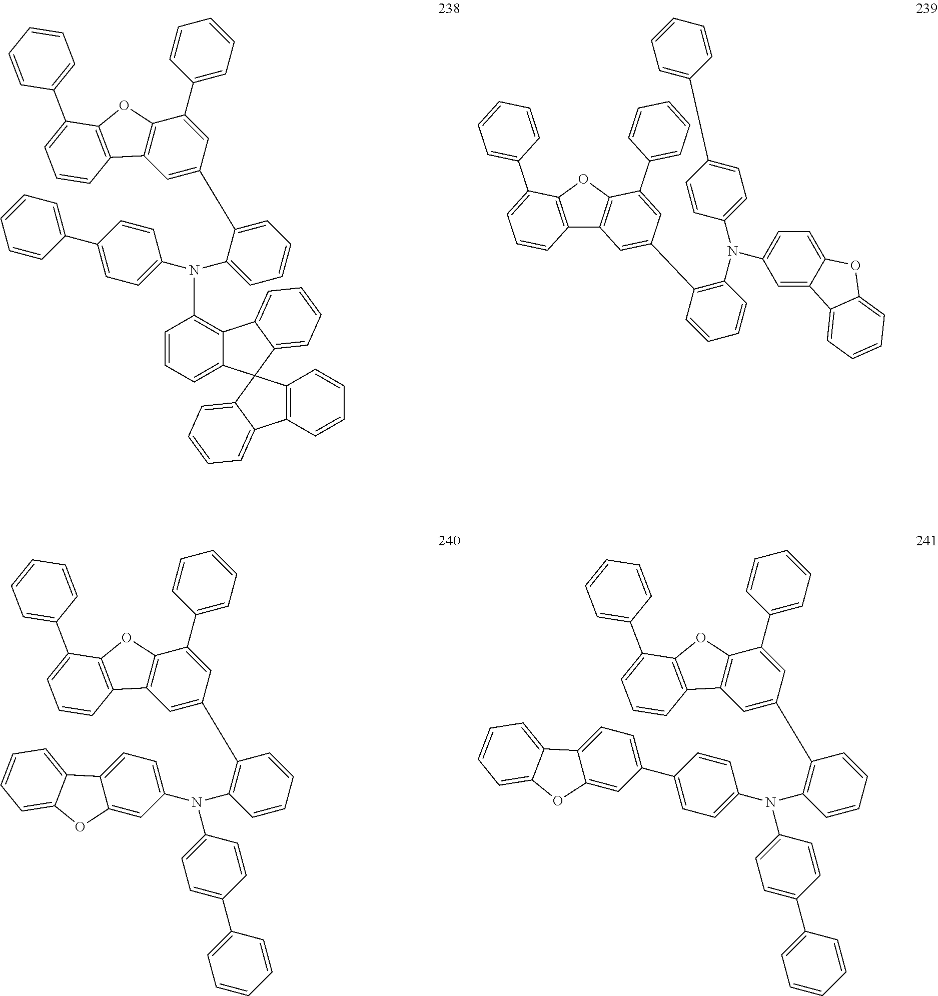 [SMILES: c1ccc(-c2ccc(N(c3ccccc3-c3cc(-c4ccccc4)c4oc5c(-c6ccccc6)cccc5c4c3)c3cccc4c3-c3ccccc3C43c4ccccc4-c4ccccc43)cc2)cc1 ; c1ccc(-c2ccc(N(c3ccc4oc5ccccc5c4c3)c3ccccc3-c3cc(-c4ccccc4)c4oc5c(-c6ccccc6)cccc5c4c3)cc2)cc1 ; c1ccc(-c2ccc(N(c3ccc4c(c3)oc3ccccc34)c3ccccc3-c3cc(-c4ccccc4)c4oc5c(-c6ccccc6)cccc5c4c3)cc2)cc1 ; c1ccc(-c2ccc(N(c3ccc(-c4ccc5c(c4)oc4ccccc45)cc3)c3ccccc3-c3cc(-c4ccccc4)c4oc5c(-c6ccccc6)cccc5c4c3)cc2)cc1]

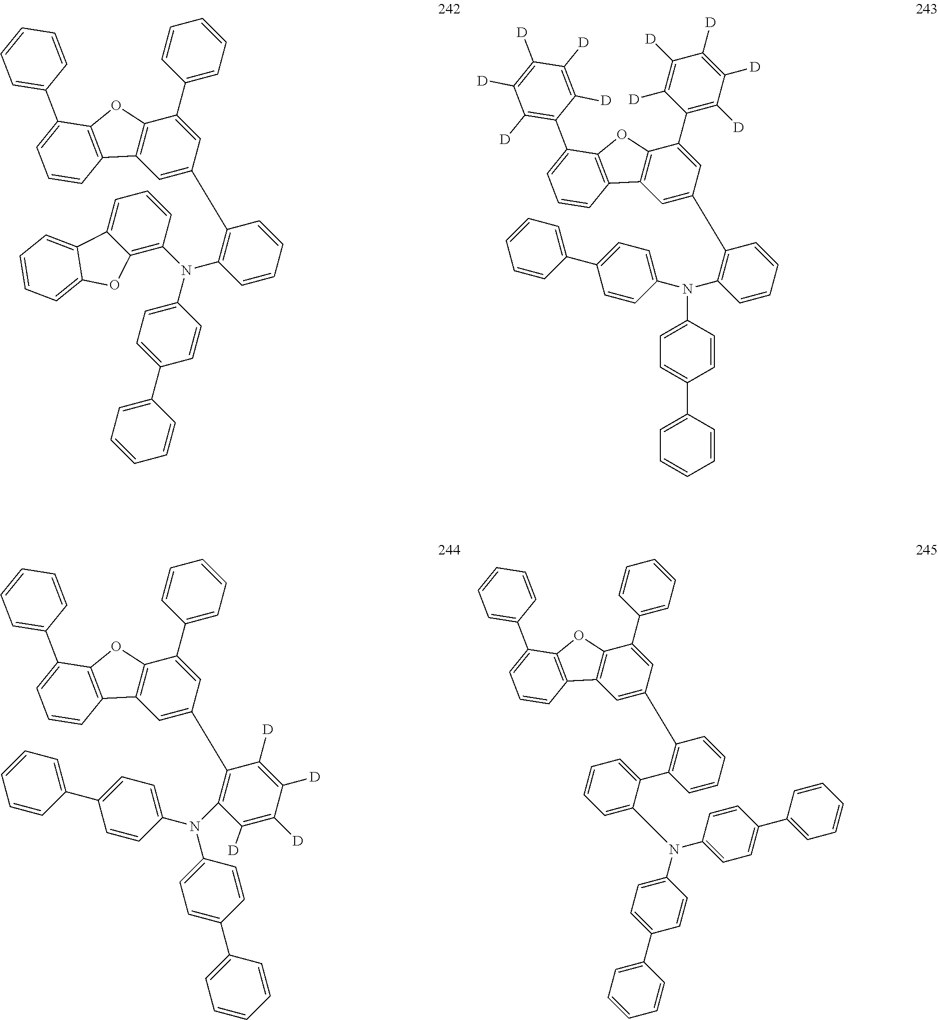 [SMILES: c1ccc(-c2ccc(N(c3ccccc3-c3cc(-c4ccccc4)c4oc5c(-c6ccccc6)cccc5c4c3)c3cccc4c3oc3ccccc34)cc2)cc1 ; [2H]c1c([2H])c([2H])c(-c2cccc3c2oc2c(-c4c([2H])c([2H])c([2H])c([2H])c4[2H])cc(-c4ccccc4N(c4ccc(-c5ccccc5)cc4)c4ccc(-c5ccccc5)cc4)cc23)c([2H])c1[2H] ; [2H]c1c([2H])c([2H])c(N(c2ccc(-c3ccccc3)cc2)c2ccc(-c3ccccc3)cc2)c(-c2cc(-c3ccccc3)c3oc4c(-c5ccccc5)cccc4c3c2)c1[2H] ; c1ccc(-c2ccc(N(c3ccc(-c4ccccc4)cc3)c3ccccc3-c3ccccc3-c3cc(-c4ccccc4)c4oc5c(-c6ccccc6)cccc5c4c3)cc2)cc1]

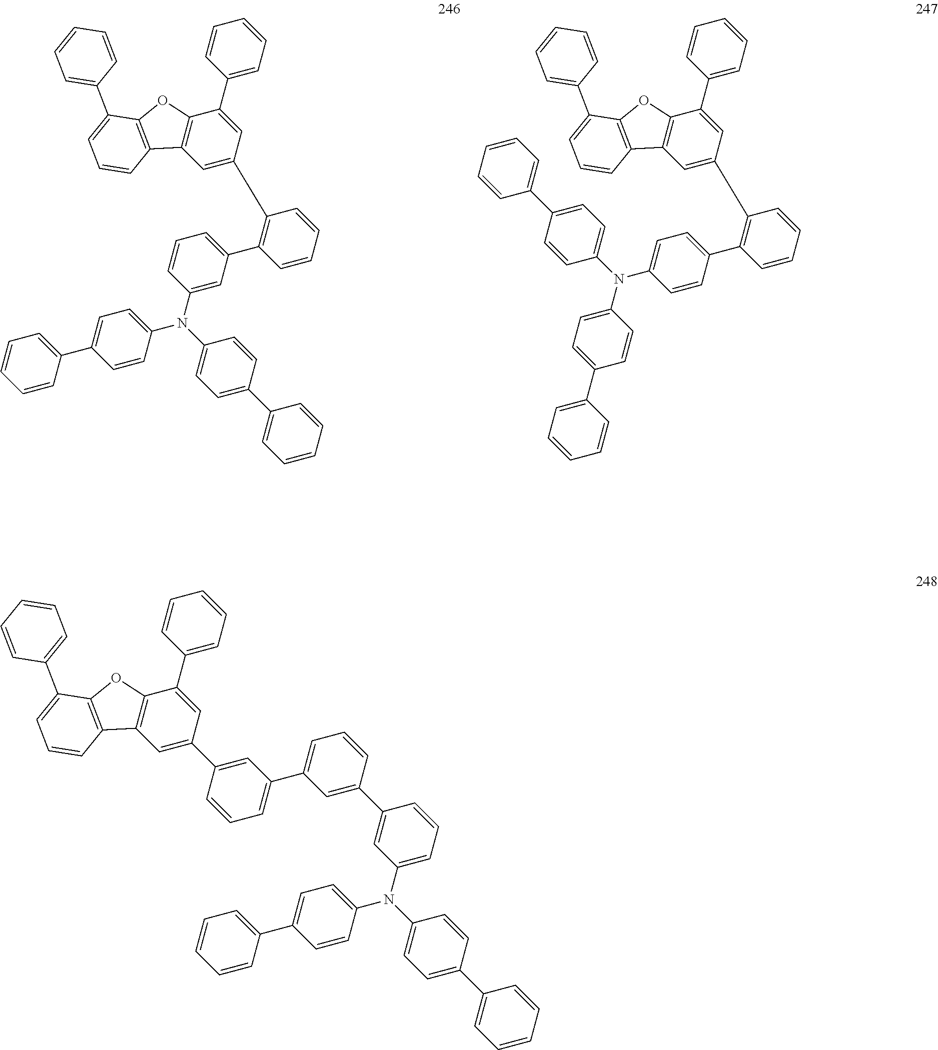 [SMILES: c1ccc(-c2ccc(N(c3ccc(-c4ccccc4)cc3)c3cccc(-c4ccccc4-c4cc(-c5ccccc5)c5oc6c(-c7ccccc7)cccc6c5c4)c3)cc2)cc1 ; c1ccc(-c2ccc(N(c3ccc(-c4ccccc4)cc3)c3ccc(-c4ccccc4-c4cc(-c5ccccc5)c5oc6c(-c7ccccc7)cccc6c5c4)cc3)cc2)cc1 ; c1ccc(-c2ccc(N(c3ccc(-c4ccccc4)cc3)c3cccc(-c4cccc(-c5cccc(-c6cc(-c7ccccc7)c7oc8c(-c9ccccc9)cccc8c7c6)c5)c4)c3)cc2)cc1]

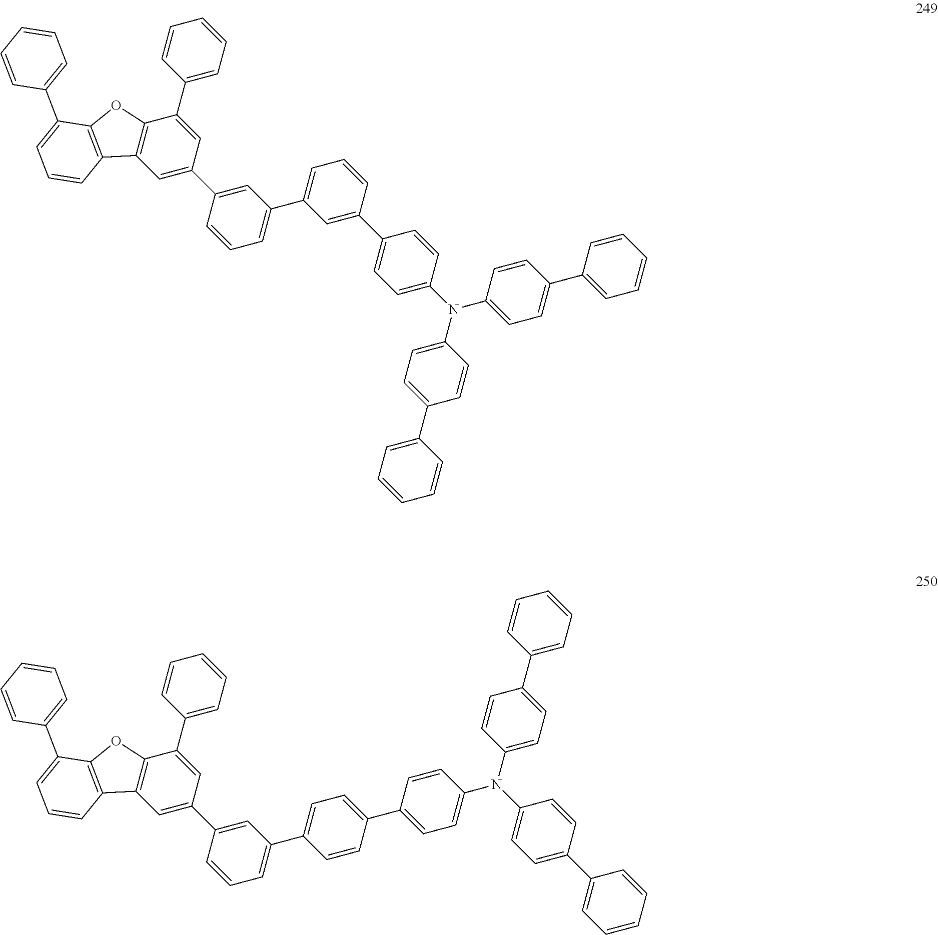 [SMILES: c1ccc(-c2ccc(N(c3ccc(-c4ccccc4)cc3)c3ccc(-c4cccc(-c5cccc(-c6cc(-c7ccccc7)c7oc8c(-c9ccccc9)cccc8c7c6)c5)c4)cc3)cc2)cc1 ; c1ccc(-c2ccc(N(c3ccc(-c4ccccc4)cc3)c3ccc(-c4ccc(-c5cccc(-c6cc(-c7ccccc7)c7oc8c(-c9ccccc9)cccc8c7c6)c5)cc4)cc3)cc2)cc1]

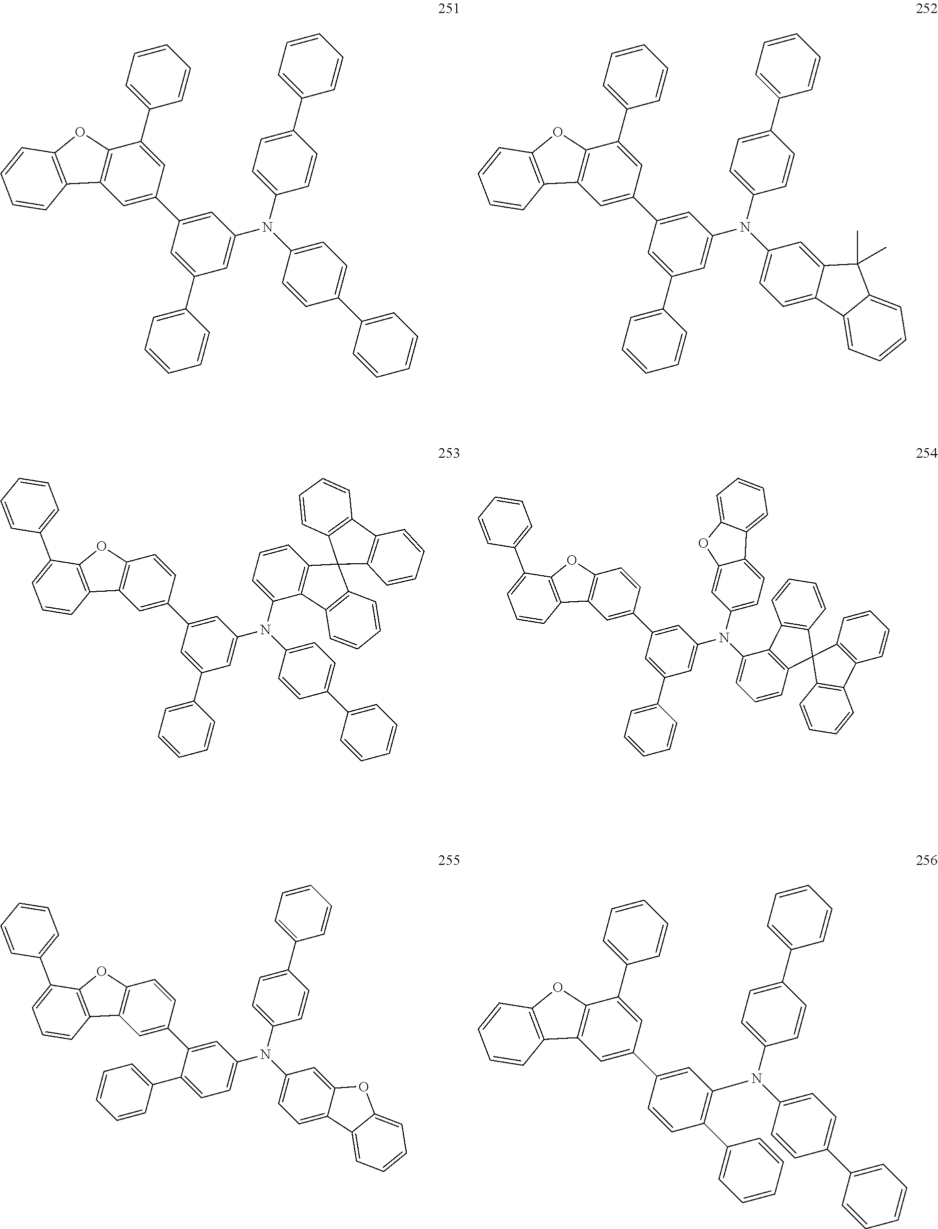

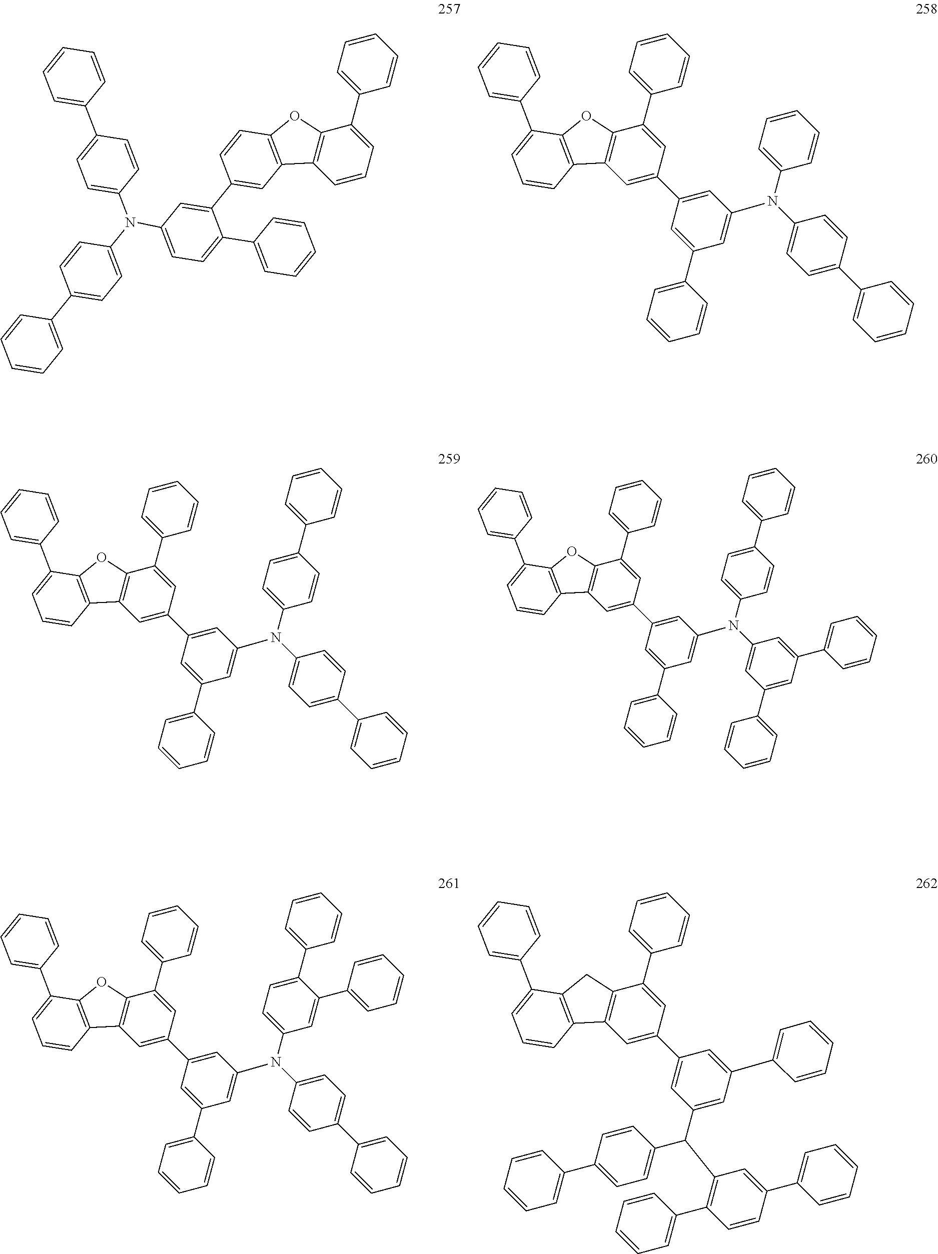

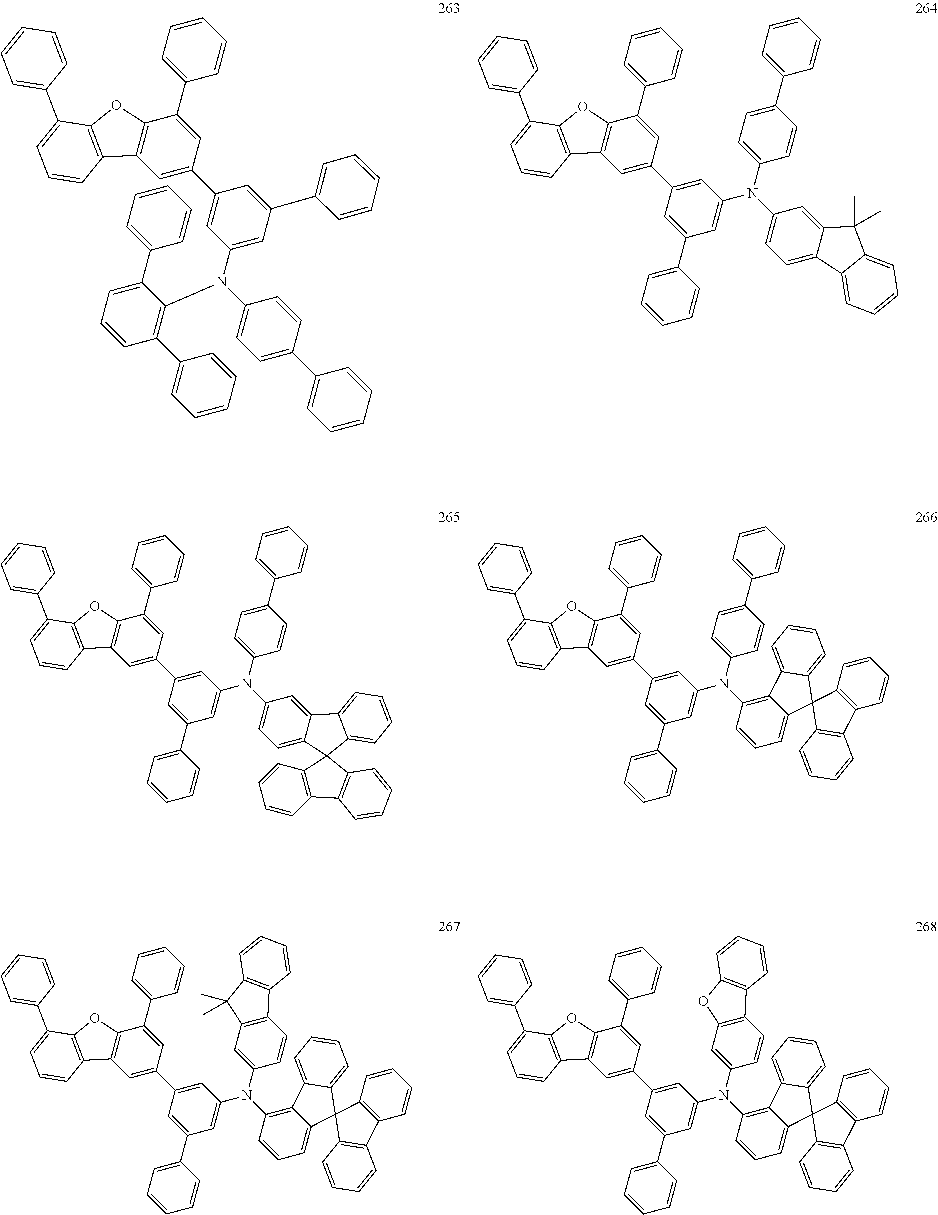

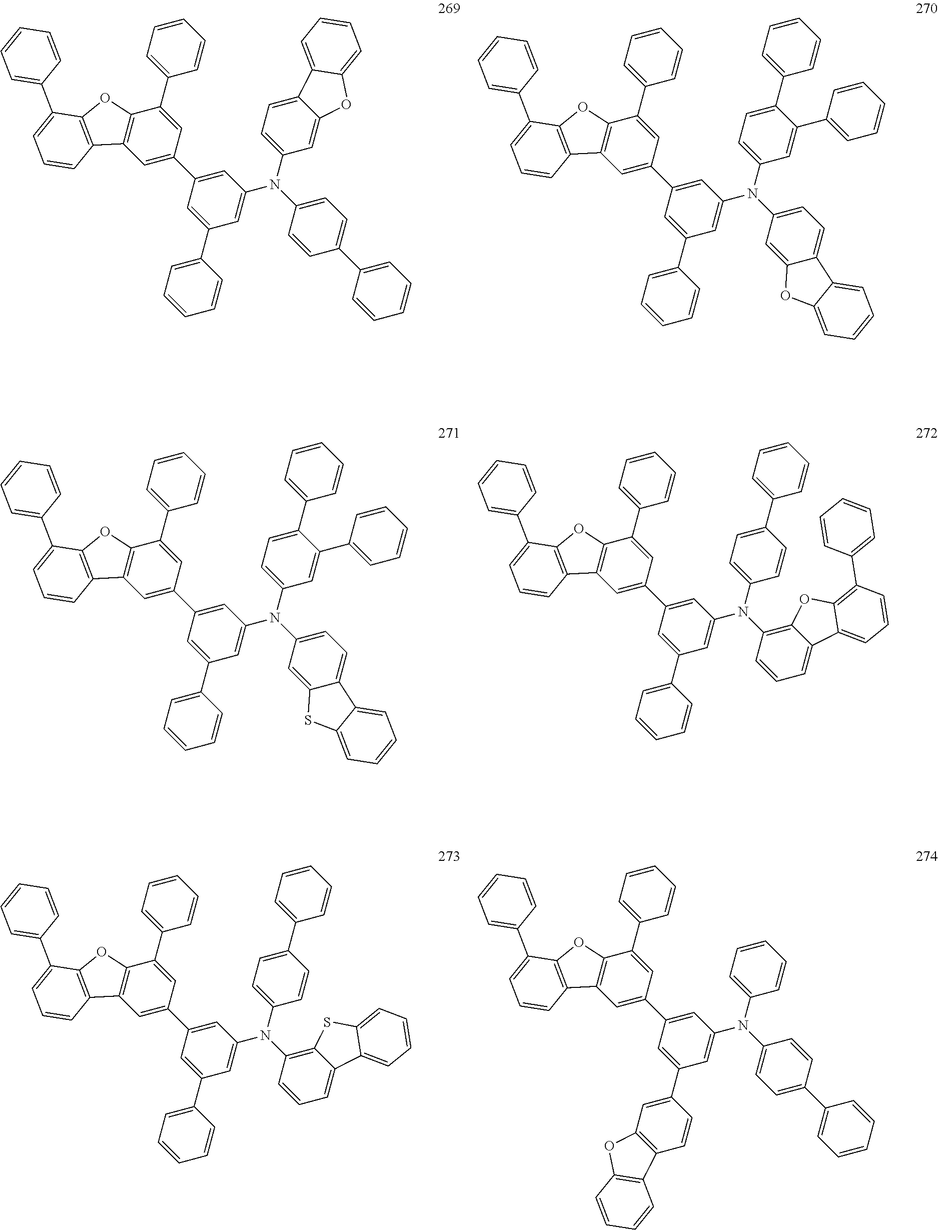

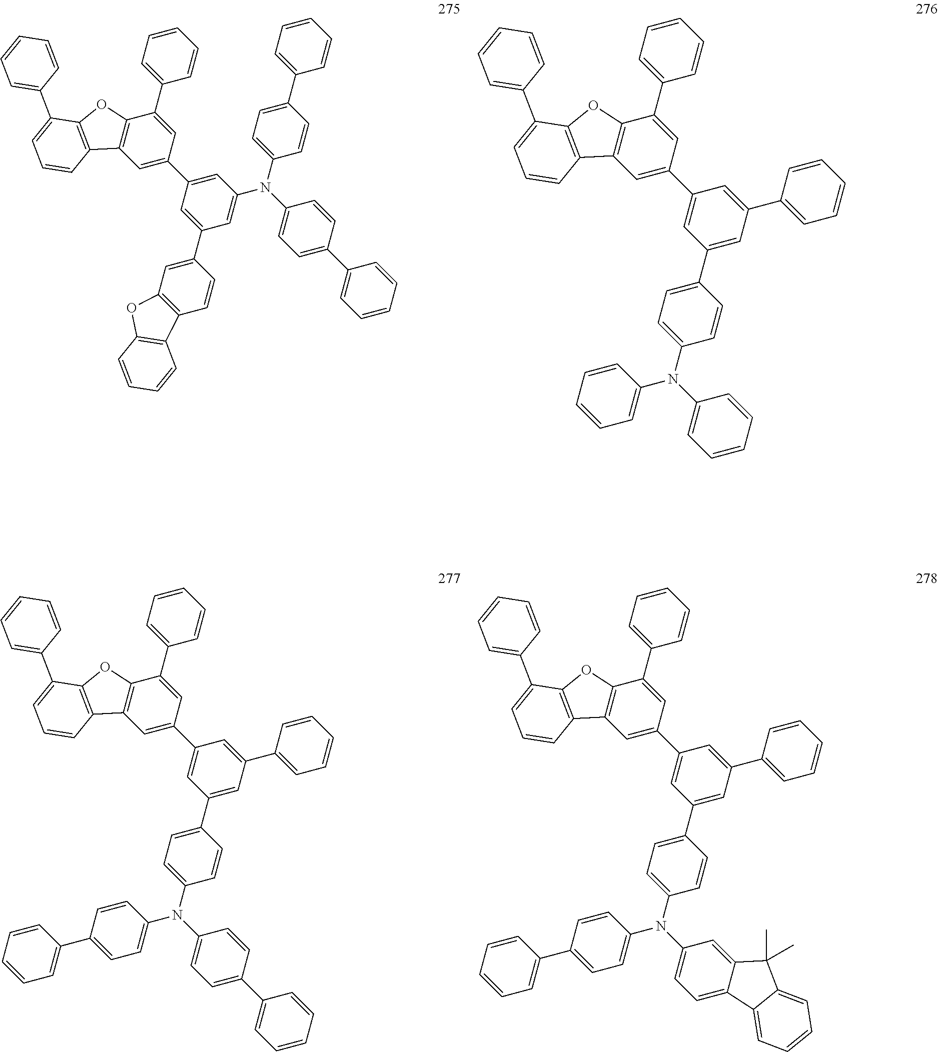 [SMILES: c1ccc(-c2ccc(N(c3ccc(-c4ccccc4)cc3)c3cc(-c4ccc5c(c4)oc4ccccc45)cc(-c4cc(-c5ccccc5)c5oc6c(-c7ccccc7)cccc6c5c4)c3)cc2)cc1 ; c1ccc(-c2cc(-c3ccc(N(c4ccccc4)c4ccccc4)cc3)cc(-c3cc(-c4ccccc4)c4oc5c(-c6ccccc6)cccc5c4c3)c2)cc1 ; c1ccc(-c2ccc(N(c3ccc(-c4ccccc4)cc3)c3ccc(-c4cc(-c5ccccc5)cc(-c5cc(-c6ccccc6)c6oc7c(-c8ccccc8)cccc7c6c5)c4)cc3)cc2)cc1 ; CC1(C)c2ccccc2-c2ccc(N(c3ccc(-c4ccccc4)cc3)c3ccc(-c4cc(-c5ccccc5)cc(-c5cc(-c6ccccc6)c6oc7c(-c8ccccc8)cccc7c6c5)c4)cc3)cc21]

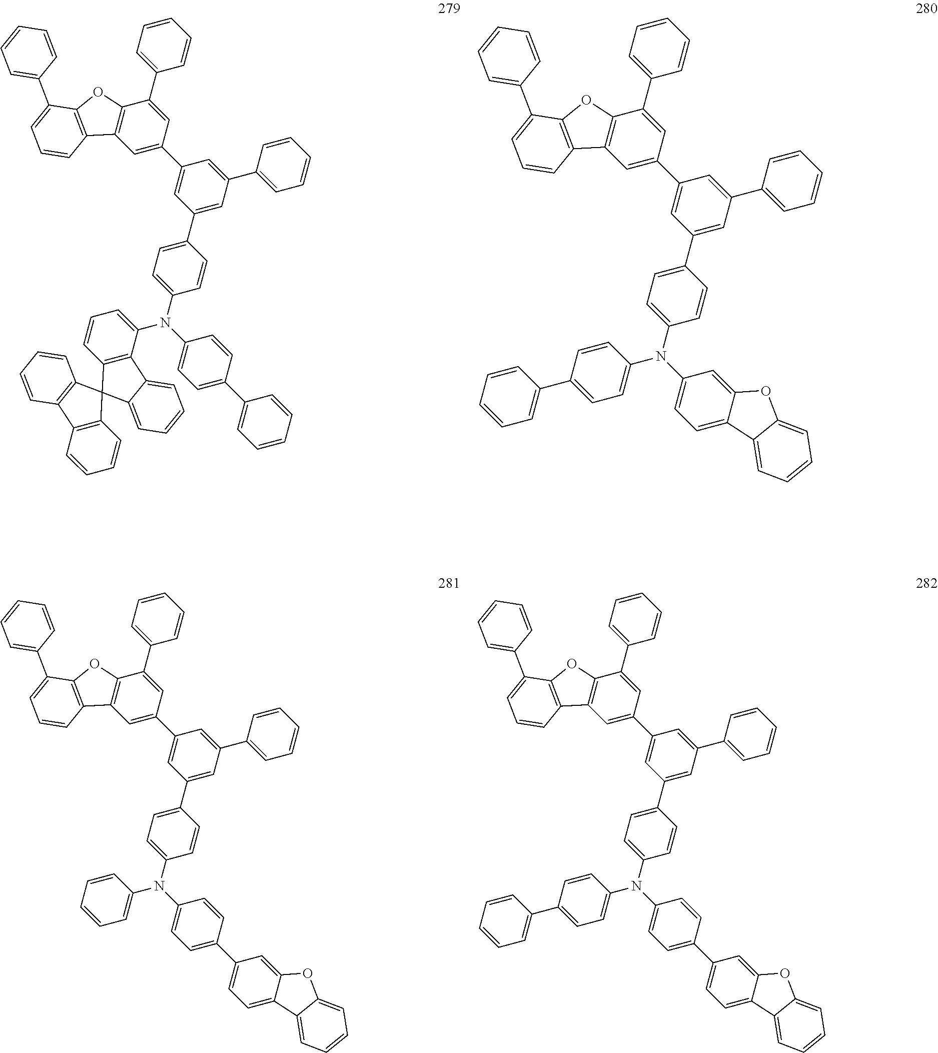

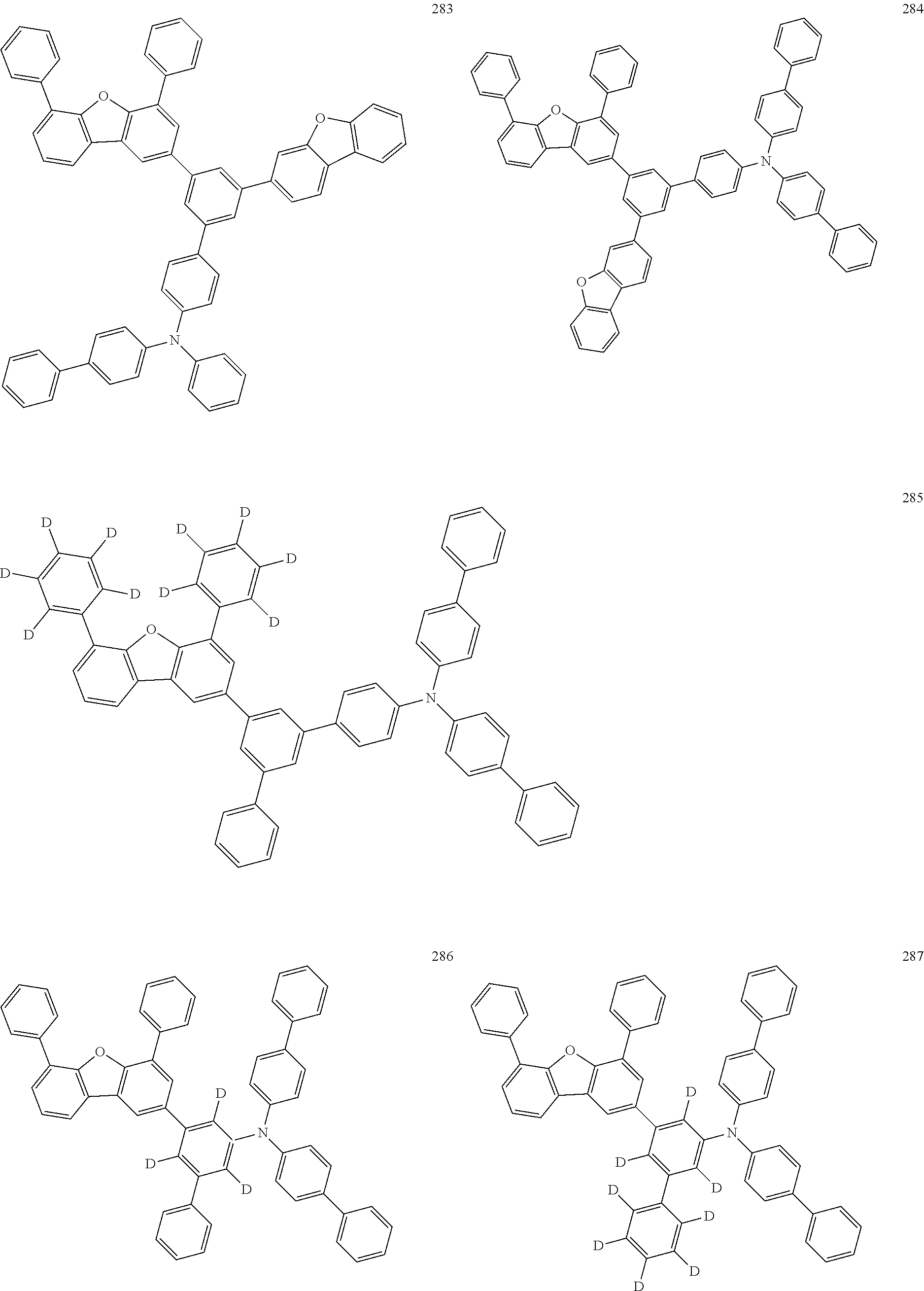

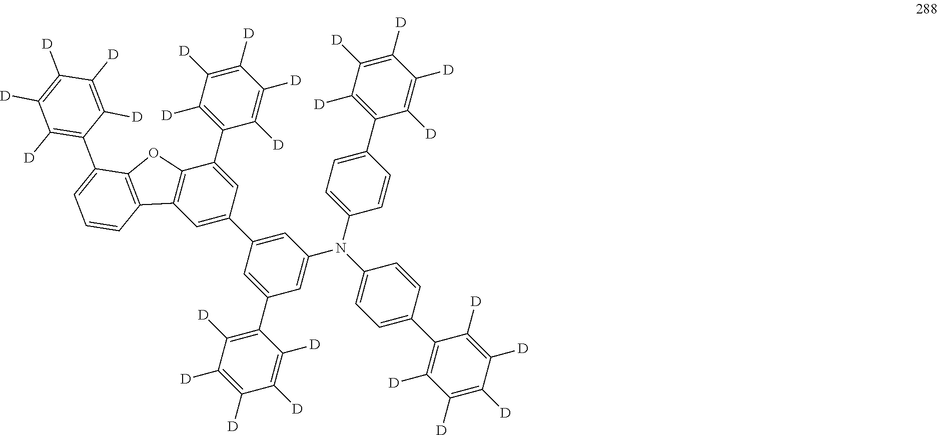 [SMILES: [2H]c1c([2H])c([2H])c(-c2ccc(N(c3ccc(-c4c([2H])c([2H])c([2H])c([2H])c4[2H])cc3)c3cc(-c4cc(-c5c([2H])c([2H])c([2H])c([2H])c5[2H])c5oc6c(-c7c([2H])c([2H])c([2H])c([2H])c7[2H])cccc6c5c4)cc(-c4c([2H])c([2H])c([2H])c([2H])c4[2H])c3)cc2)c([2H])c1[2H]]

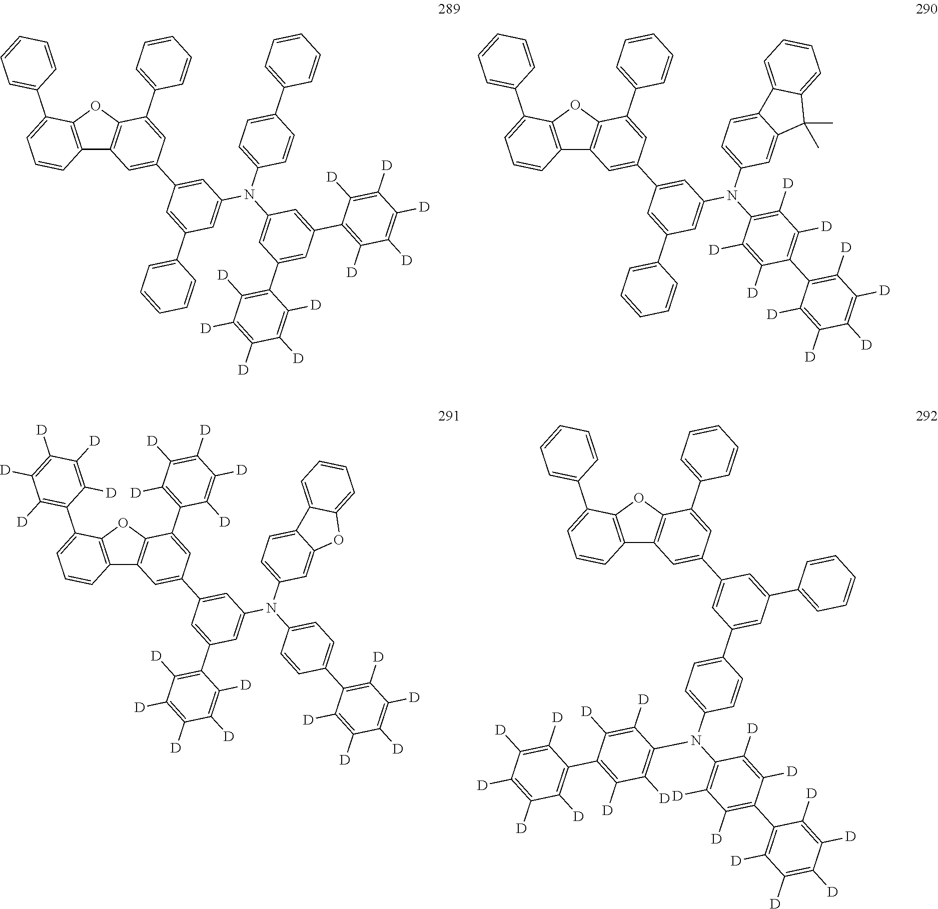

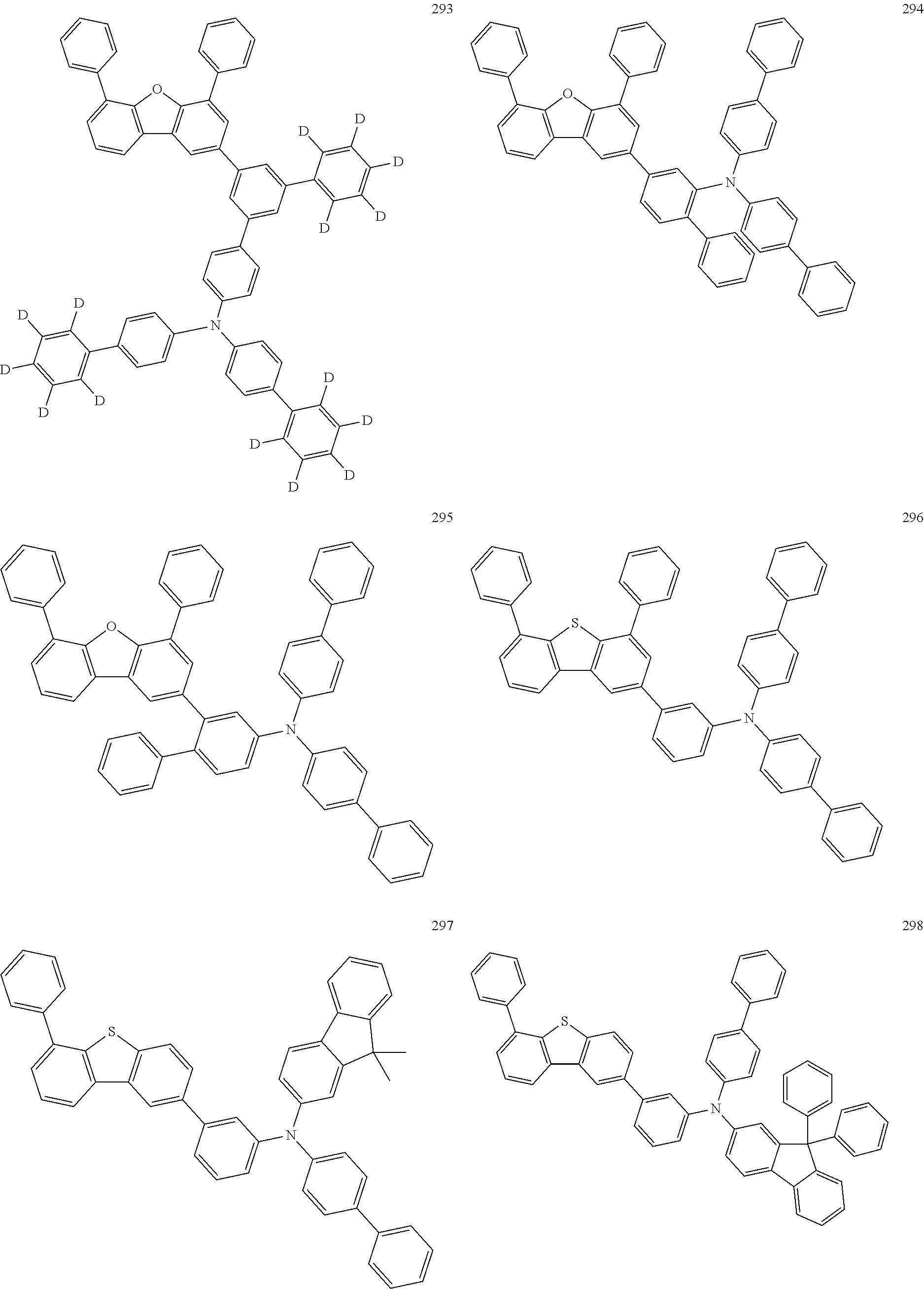

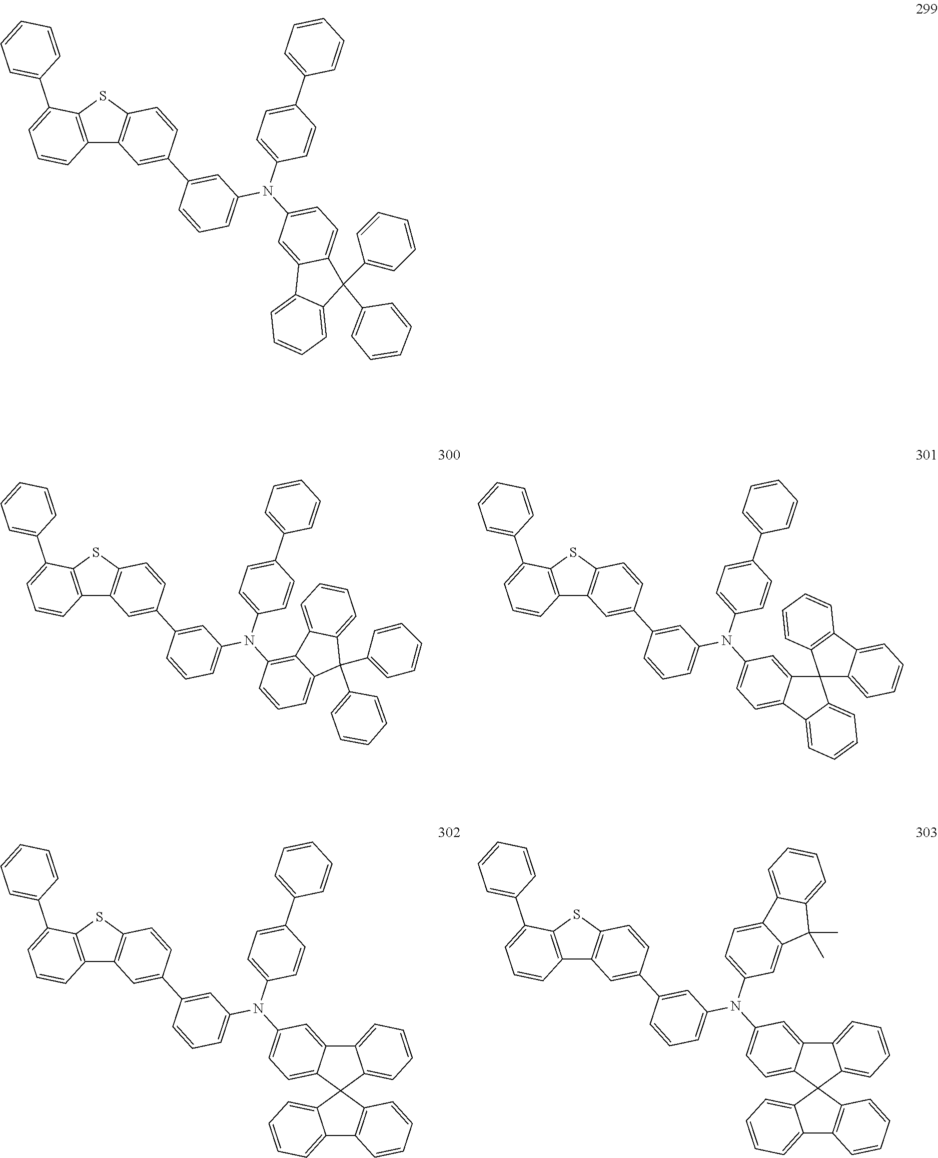

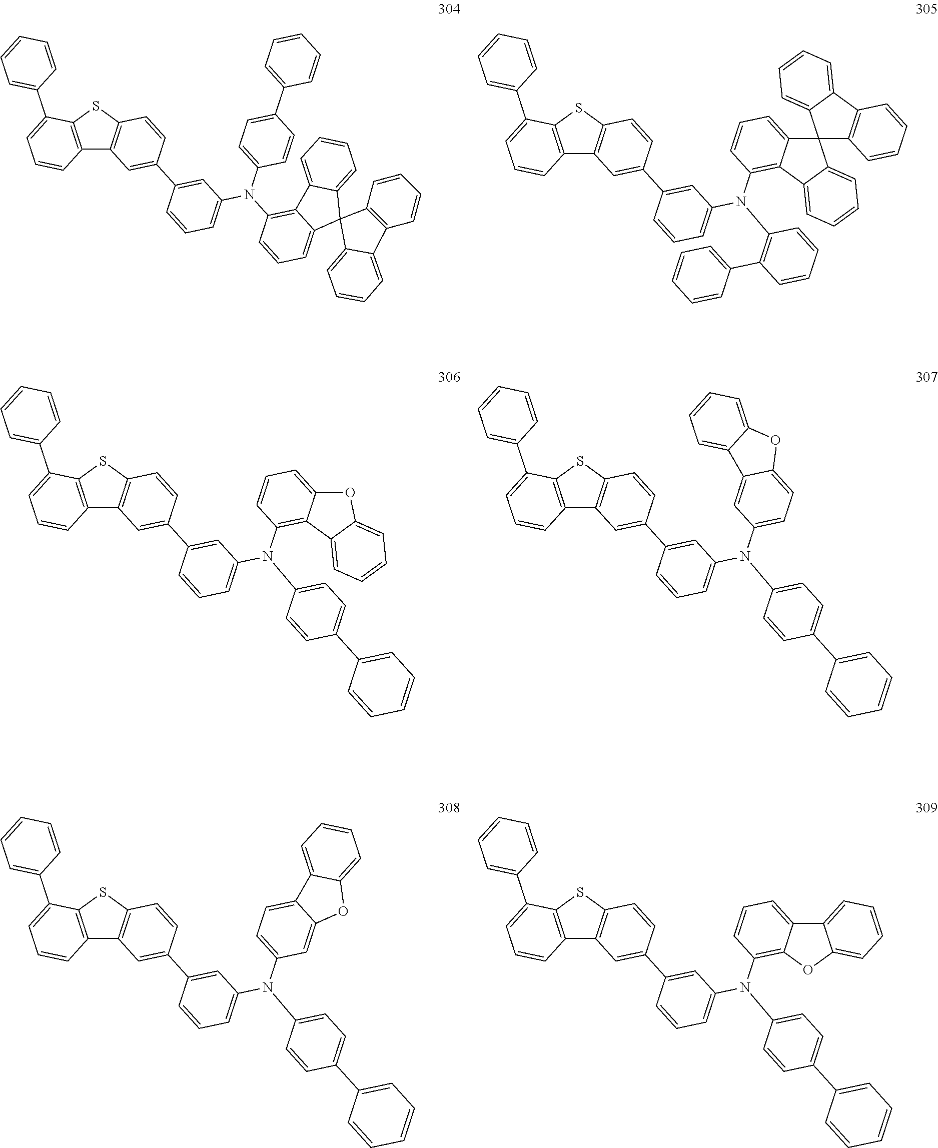

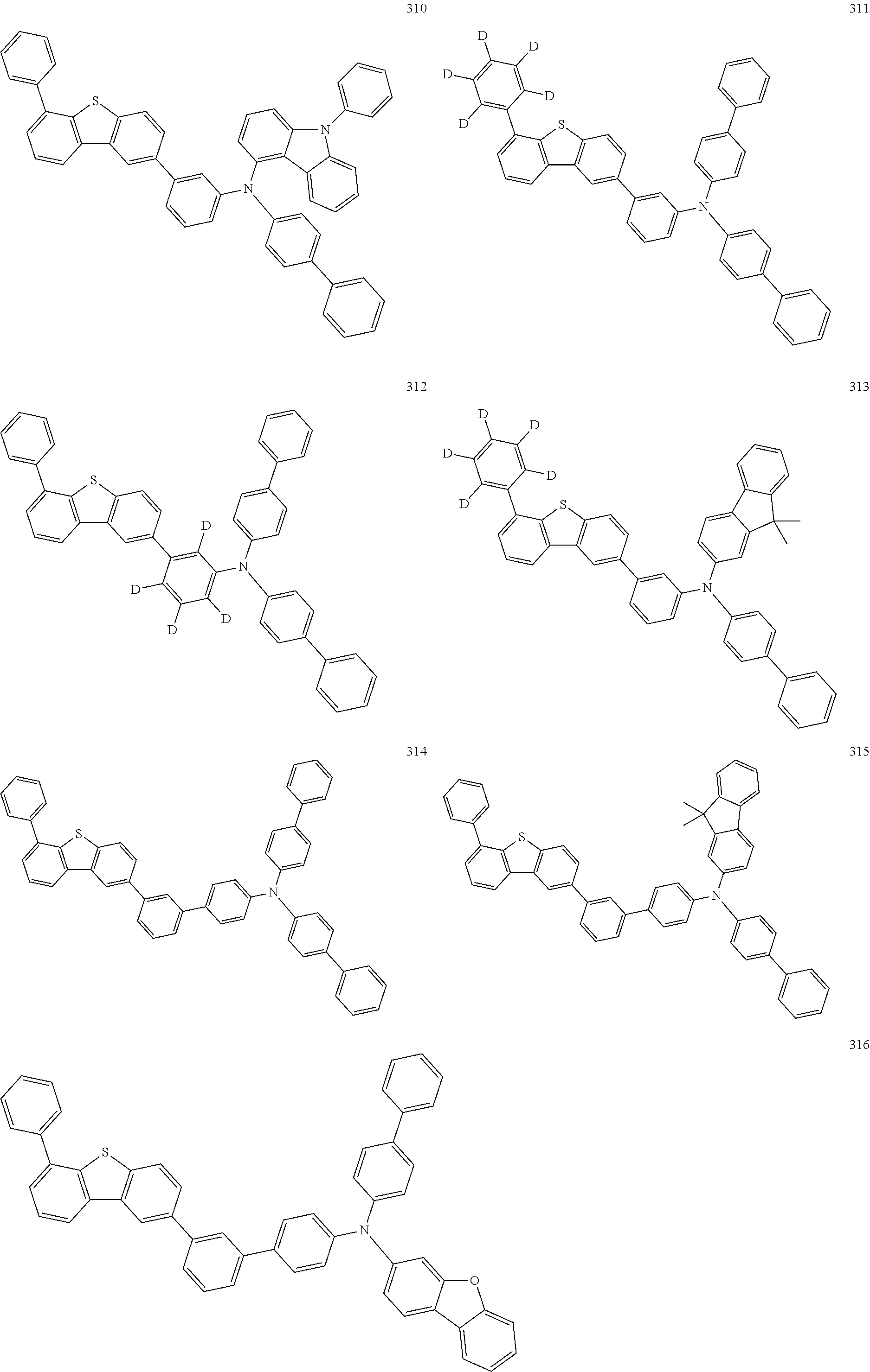

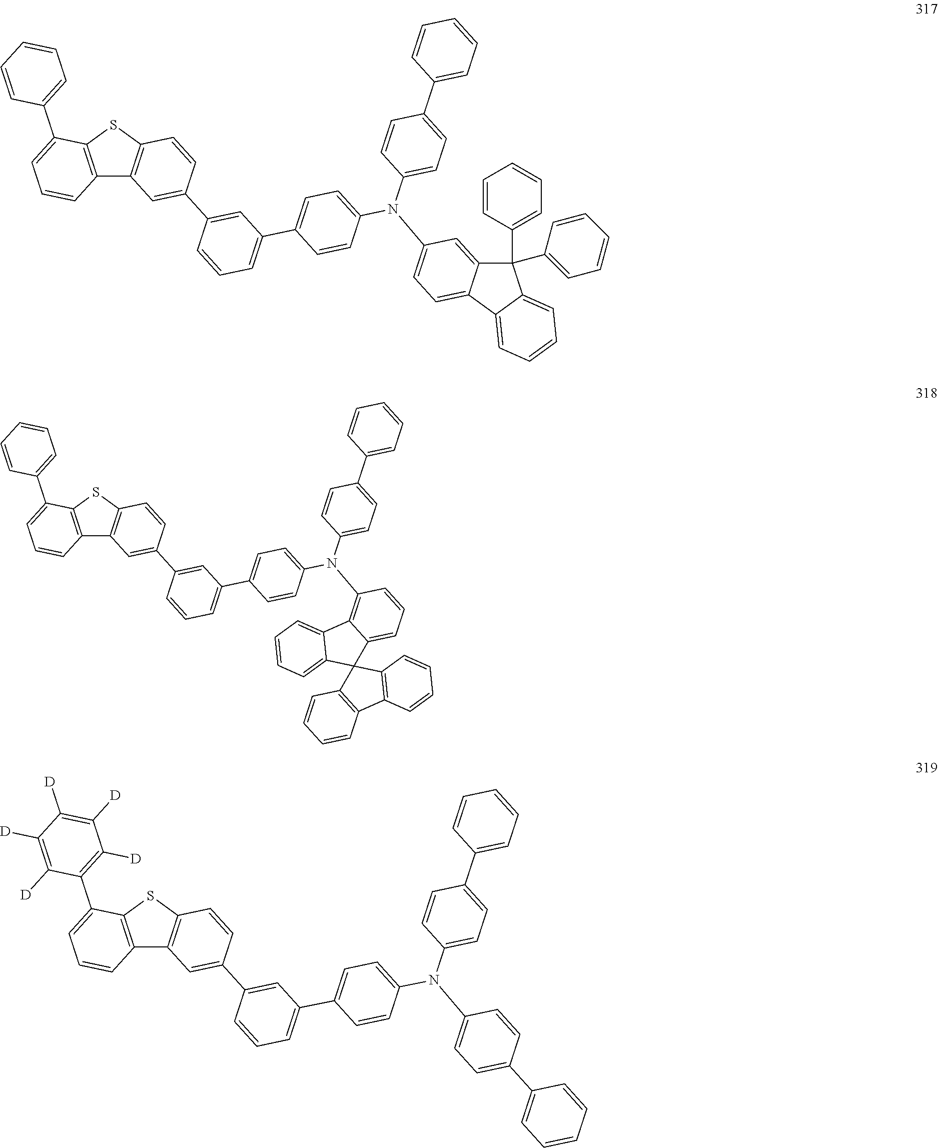 [SMILES: c1ccc(-c2ccc(N(c3ccc(-c4cccc(-c5ccc6sc7c(-c8ccccc8)cccc7c6c5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6sc7c(-c8ccccc8)cccc7c6c5)c4)cc3)c3cccc4c3-c3ccccc3C43c4ccccc4-c4ccccc43)cc2)cc1 ; [2H]c1c([2H])c([2H])c(-c2cccc3c2sc2ccc(-c4cccc(-c5ccc(N(c6ccc(-c7ccccc7)cc6)c6ccc(-c7ccccc7)cc6)cc5)c4)cc23)c([2H])c1[2H]]

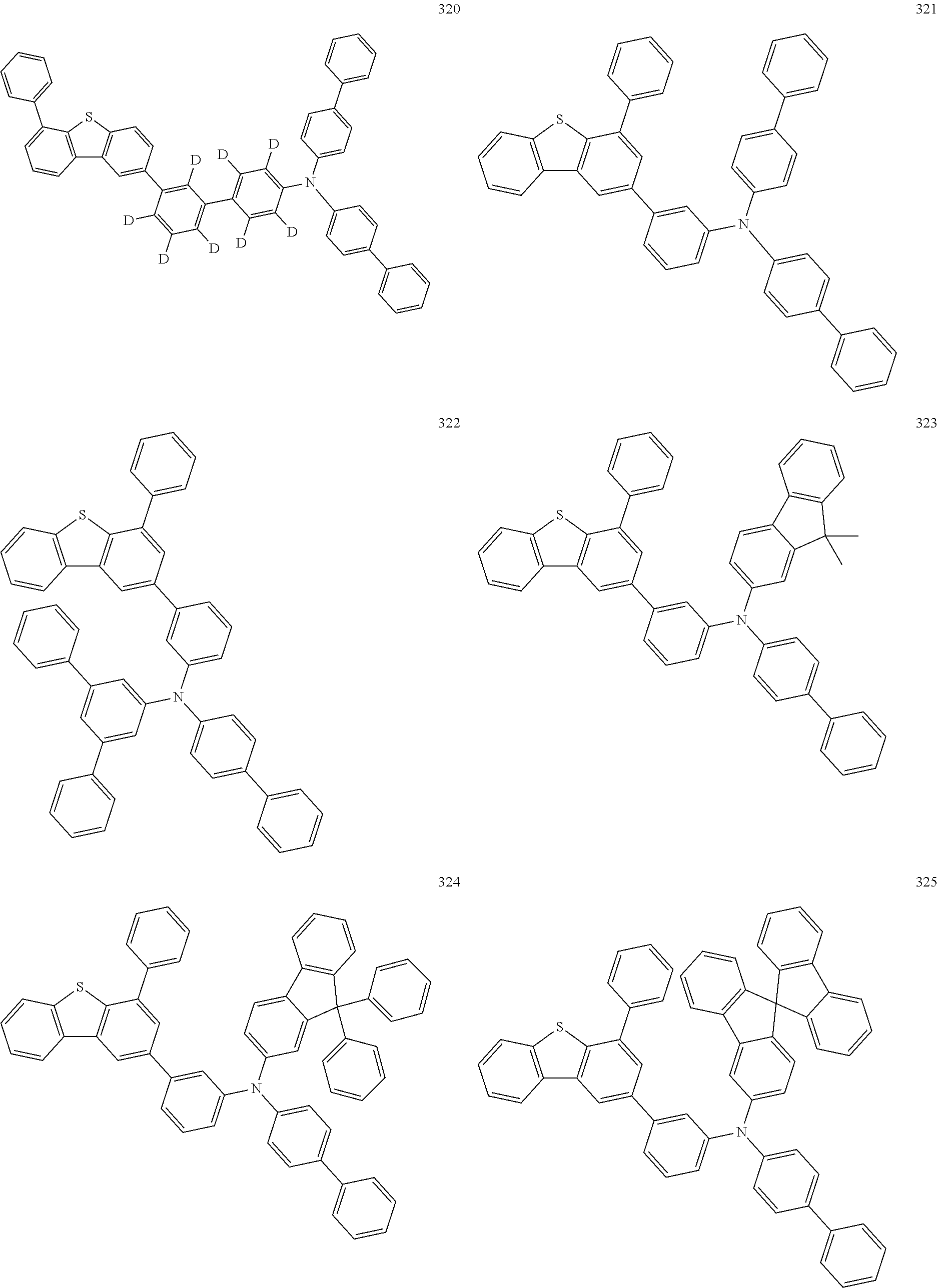

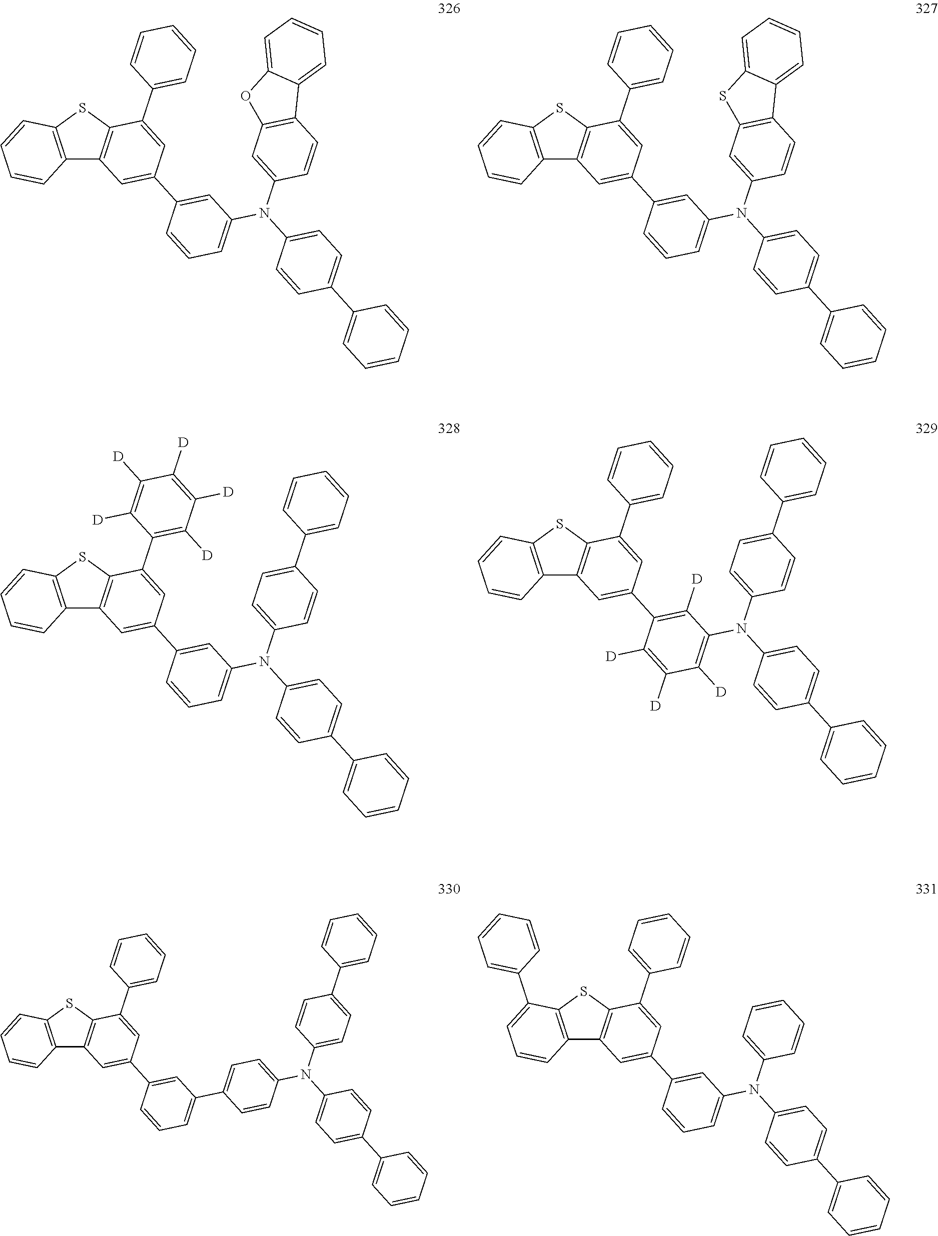

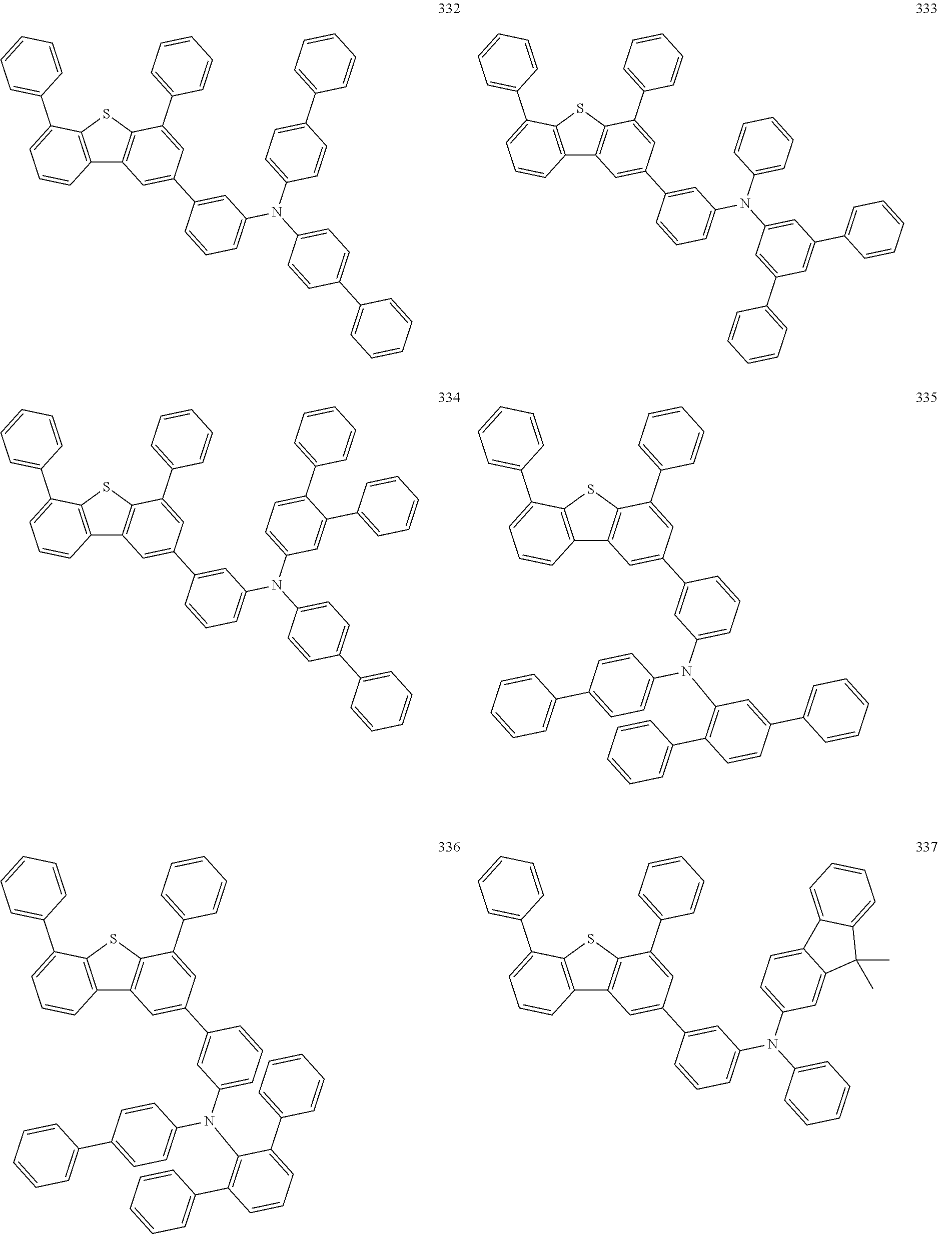

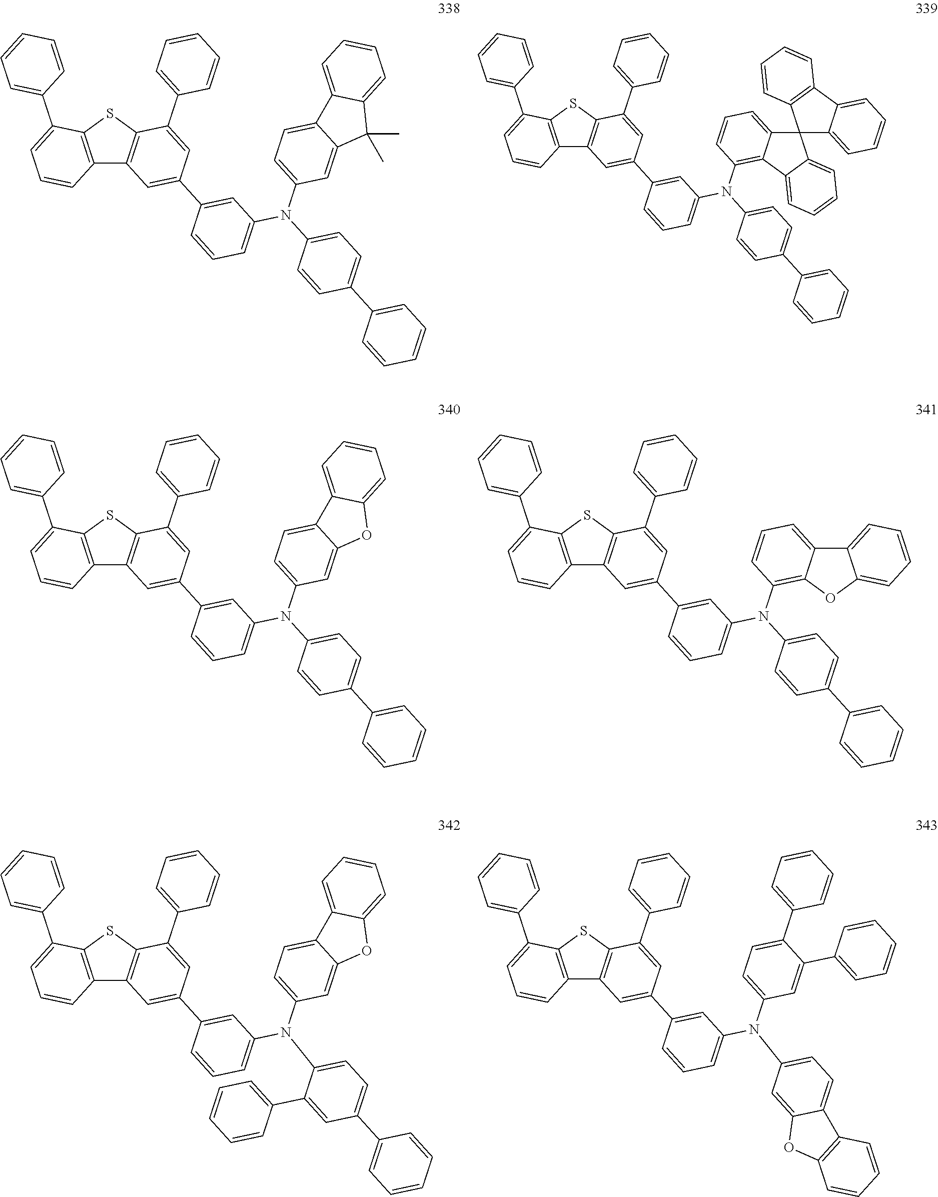

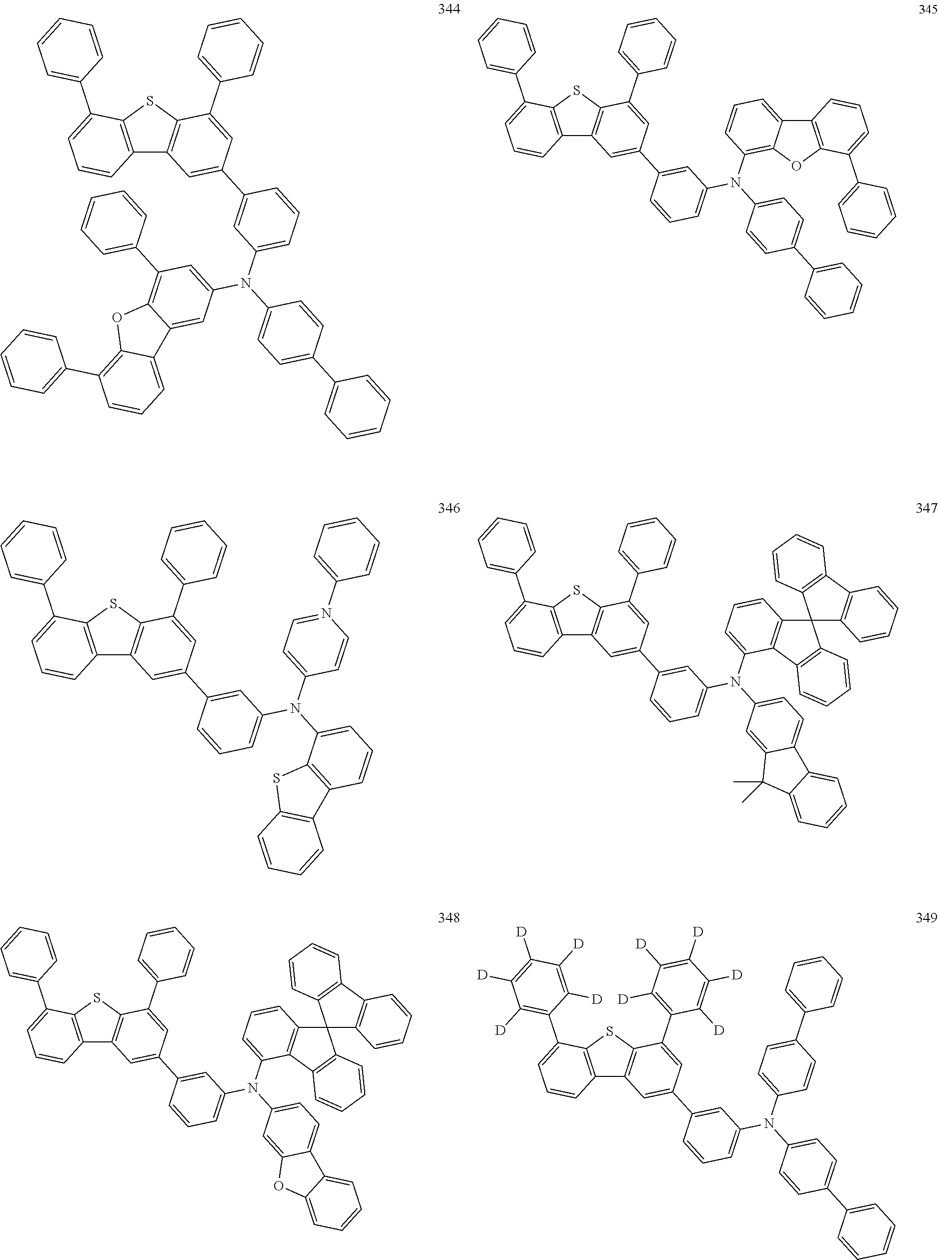

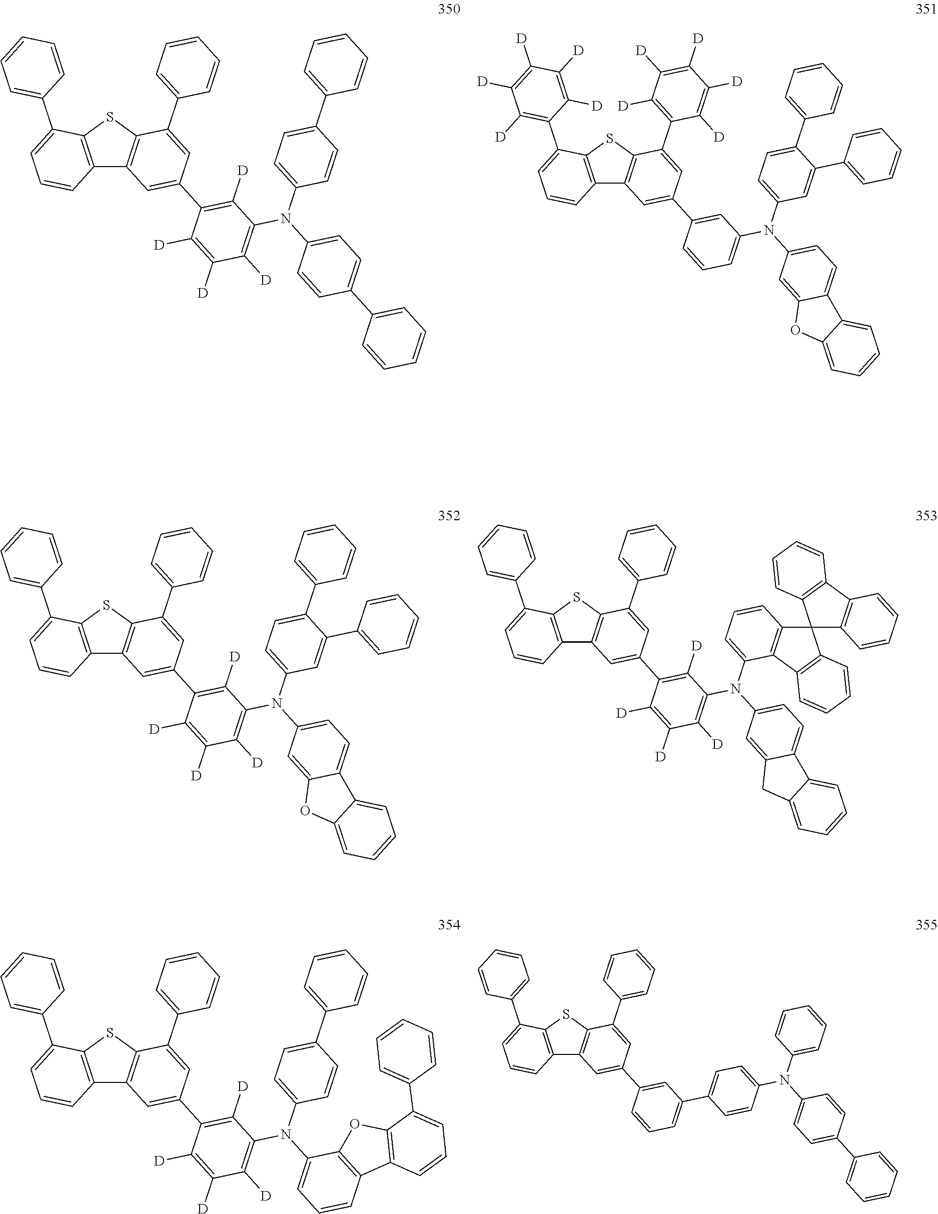

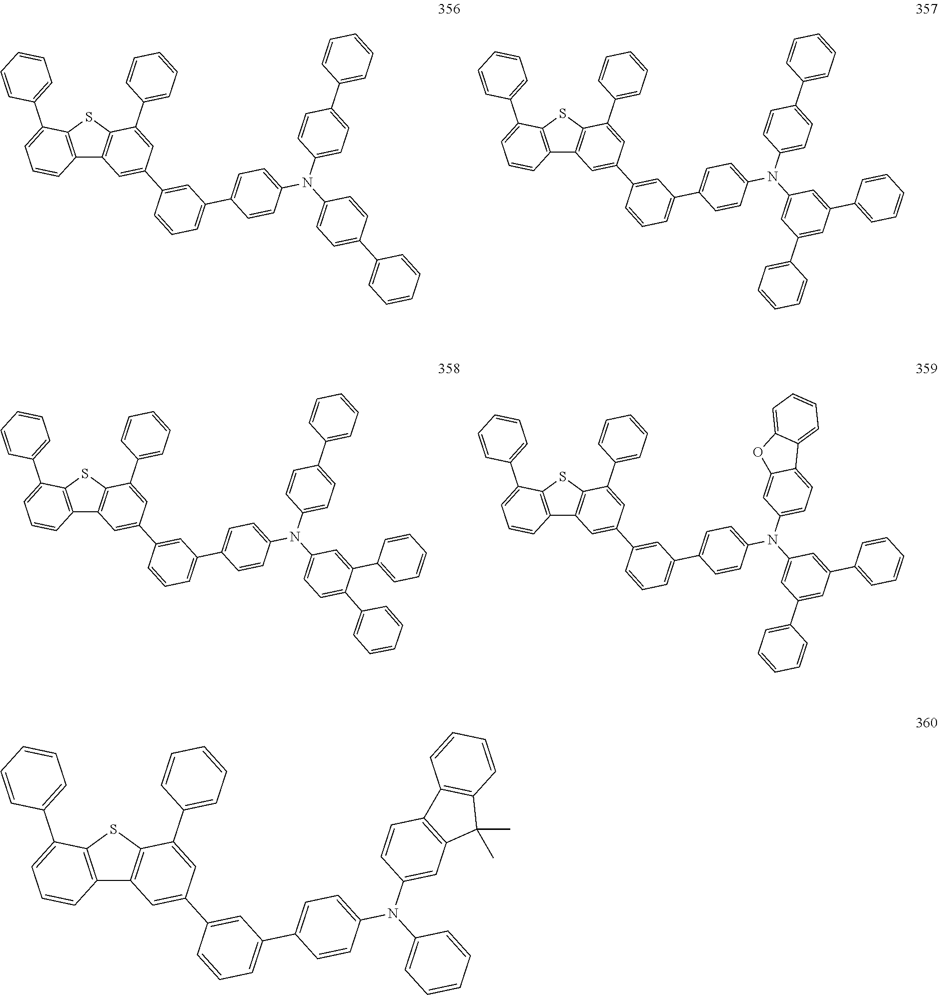

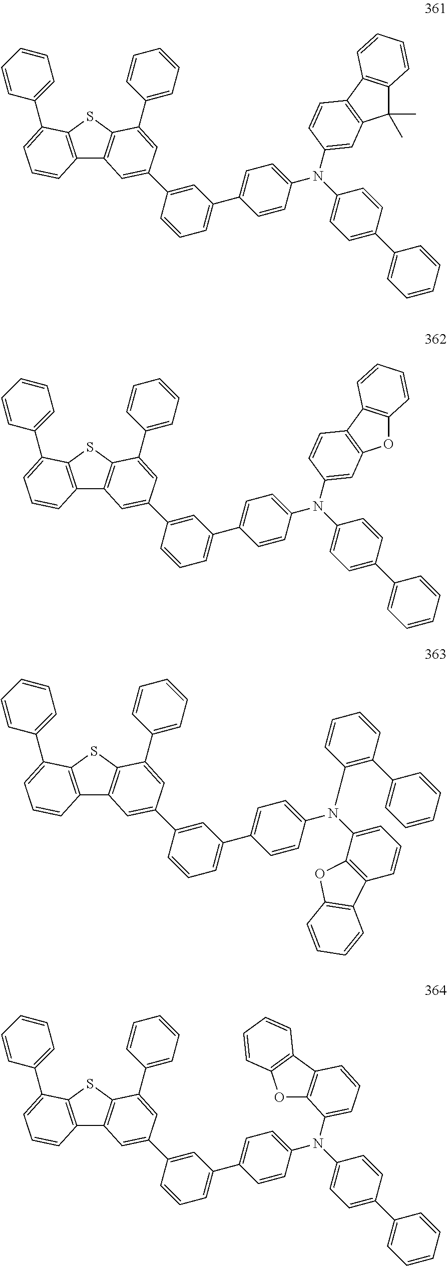 [SMILES: CC1(C)c2ccccc2-c2ccc(N(c3ccc(-c4ccccc4)cc3)c3ccc(-c4cccc(-c5cc(-c6ccccc6)c6sc7c(-c8ccccc8)cccc7c6c5)c4)cc3)cc21 ; c1ccc(-c2ccc(N(c3ccc(-c4cccc(-c5cc(-c6ccccc6)c6sc7c(-c8ccccc8)cccc7c6c5)c4)cc3)c3ccc4c(c3)oc3ccccc34)cc2)cc1 ; c1ccc(-c2ccccc2N(c2ccc(-c3cccc(-c4cc(-c5ccccc5)c5sc6c(-c7ccccc7)cccc6c5c4)c3)cc2)c2cccc3c2oc2ccccc23)cc1 ; c1ccc(-c2ccc(N(c3ccc(-c4cccc(-c5cc(-c6ccccc6)c6sc7c(-c8ccccc8)cccc7c6c5)c4)cc3)c3cccc4c3oc3ccccc34)cc2)cc1]

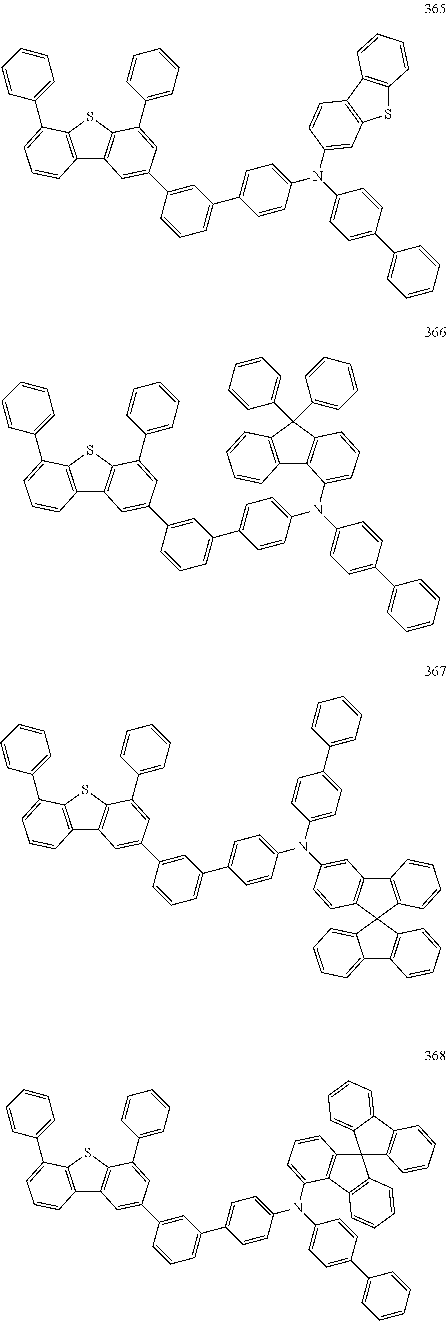 [SMILES: c1ccc(-c2ccc(N(c3ccc(-c4cccc(-c5cc(-c6ccccc6)c6sc7c(-c8ccccc8)cccc7c6c5)c4)cc3)c3ccc4c(c3)sc3ccccc34)cc2)cc1 ; c1ccc(-c2ccc(N(c3ccc(-c4cccc(-c5cc(-c6ccccc6)c6sc7c(-c8ccccc8)cccc7c6c5)c4)cc3)c3cccc4c3-c3ccccc3C4(c3ccccc3)c3ccccc3)cc2)cc1 ; c1ccc(-c2ccc(N(c3ccc(-c4cccc(-c5cc(-c6ccccc6)c6sc7c(-c8ccccc8)cccc7c6c5)c4)cc3)c3ccc4c(c3)-c3ccccc3C43c4ccccc4-c4ccccc43)cc2)cc1 ; c1ccc(-c2ccc(N(c3ccc(-c4cccc(-c5cc(-c6ccccc6)c6sc7c(-c8ccccc8)cccc7c6c5)c4)cc3)c3cccc4c3-c3ccccc3C43c4ccccc4-c4ccccc43)cc2)cc1]

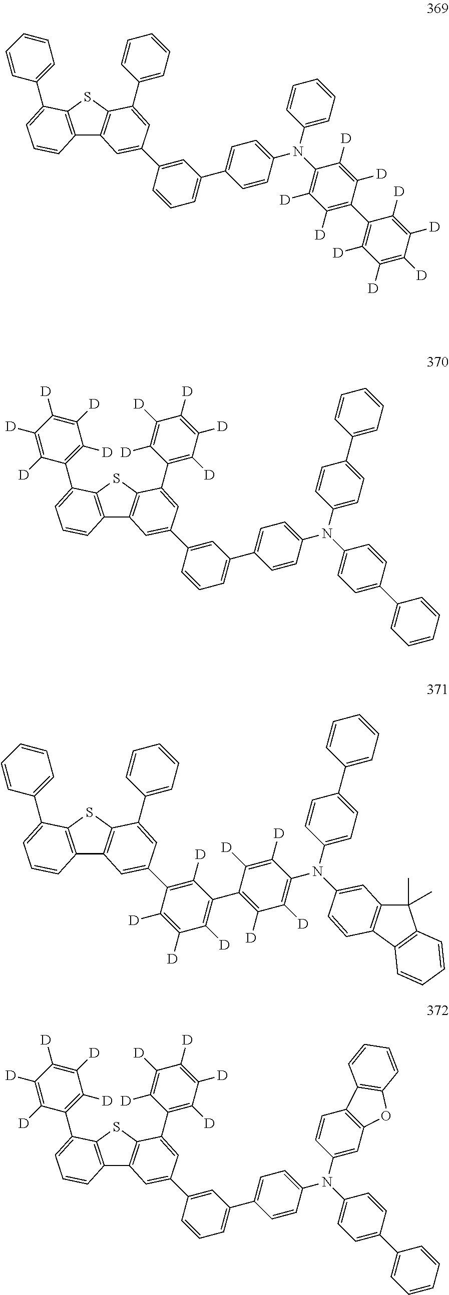

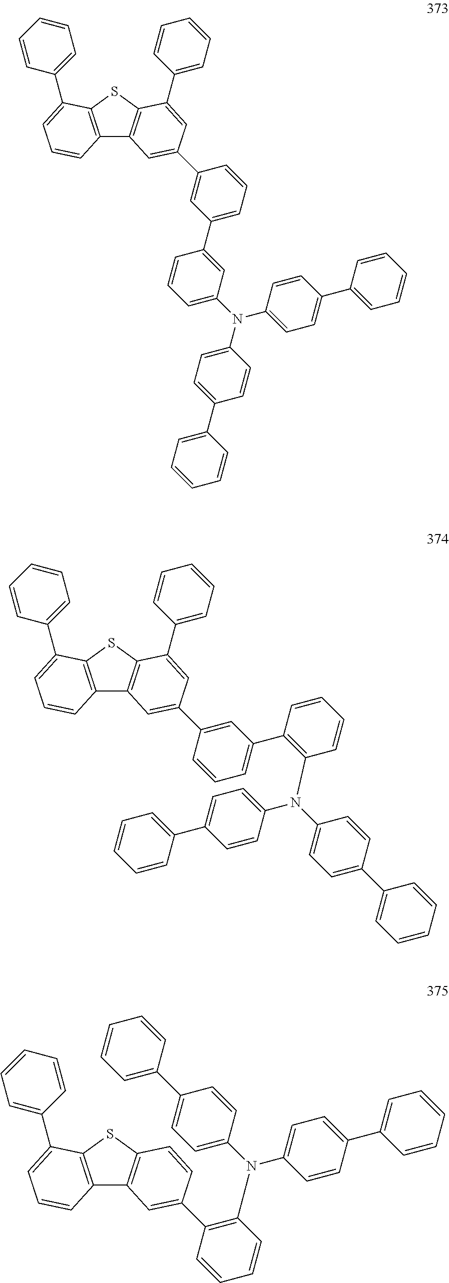 [SMILES: c1ccc(-c2ccc(N(c3ccc(-c4ccccc4)cc3)c3cccc(-c4cccc(-c5cc(-c6ccccc6)c6sc7c(-c8ccccc8)cccc7c6c5)c4)c3)cc2)cc1 ; c1ccc(-c2ccc(N(c3ccc(-c4ccccc4)cc3)c3ccccc3-c3cccc(-c4cc(-c5ccccc5)c5sc6c(-c7ccccc7)cccc6c5c4)c3)cc2)cc1 ; c1ccc(-c2ccc(N(c3ccc(-c4ccccc4)cc3)c3ccccc3-c3ccc4sc5c(-c6ccccc6)cccc5c4c3)cc2)cc1]

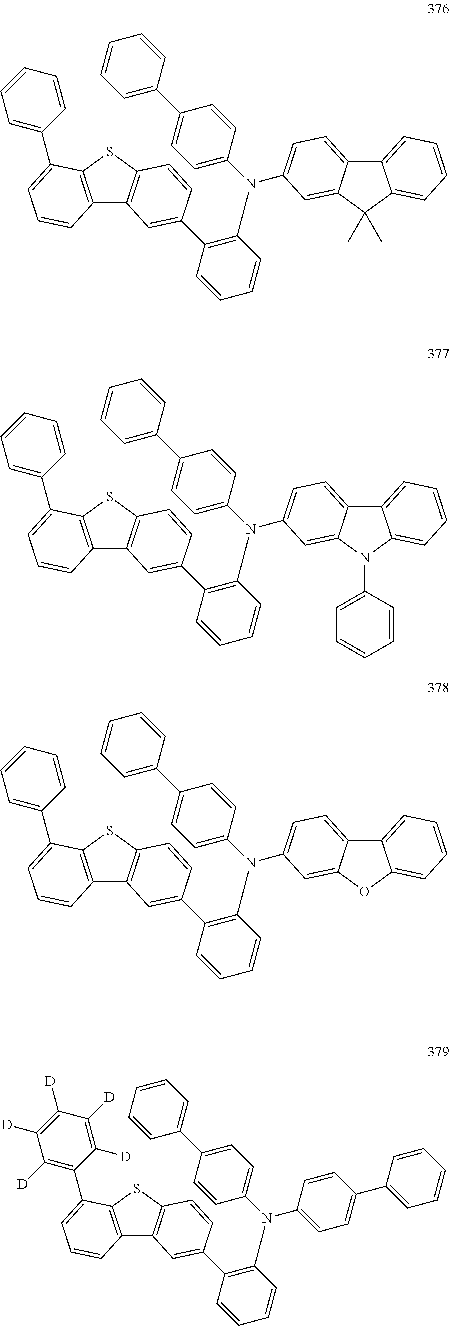 [SMILES: CC1(C)c2ccccc2-c2ccc(N(c3ccc(-c4ccccc4)cc3)c3ccccc3-c3ccc4sc5c(-c6ccccc6)cccc5c4c3)cc21 ; c1ccc(-c2ccc(N(c3ccc4c5ccccc5n(-c5ccccc5)c4c3)c3ccccc3-c3ccc4sc5c(-c6ccccc6)cccc5c4c3)cc2)cc1 ; c1ccc(-c2ccc(N(c3ccc4c(c3)oc3ccccc34)c3ccccc3-c3ccc4sc5c(-c6ccccc6)cccc5c4c3)cc2)cc1 ; [2H]c1c([2H])c([2H])c(-c2cccc3c2sc2ccc(-c4ccccc4N(c4ccc(-c5ccccc5)cc4)c4ccc(-c5ccccc5)cc4)cc23)c([2H])c1[2H]]

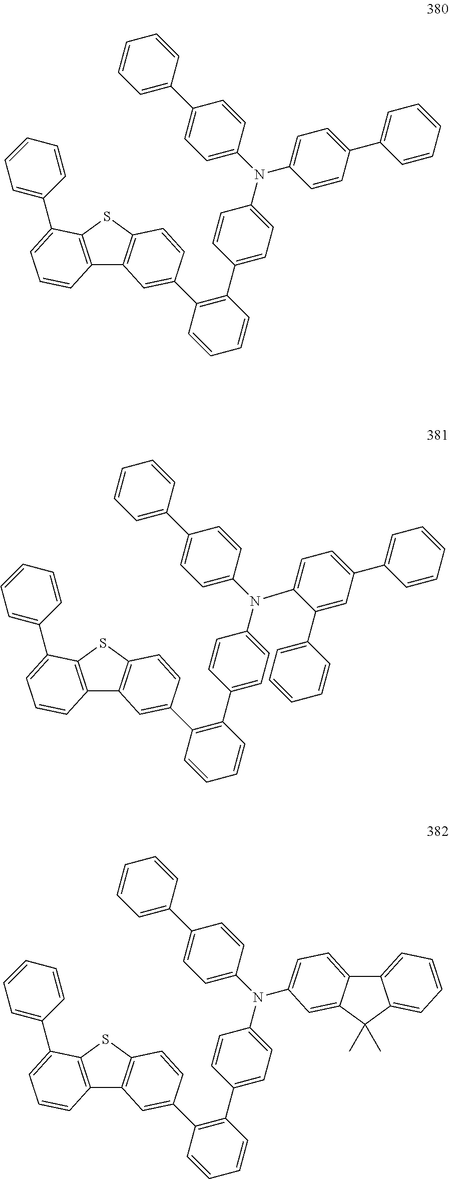 [SMILES: c1ccc(-c2ccc(N(c3ccc(-c4ccccc4)cc3)c3ccc(-c4ccccc4-c4ccc5sc6c(-c7ccccc7)cccc6c5c4)cc3)cc2)cc1 ; c1ccc(-c2ccc(N(c3ccc(-c4ccccc4-c4ccc5sc6c(-c7ccccc7)cccc6c5c4)cc3)c3ccc(-c4ccccc4)cc3-c3ccccc3)cc2)cc1 ; CC1(C)c2ccccc2-c2ccc(N(c3ccc(-c4ccccc4)cc3)c3ccc(-c4ccccc4-c4ccc5sc6c(-c7ccccc7)cccc6c5c4)cc3)cc21]

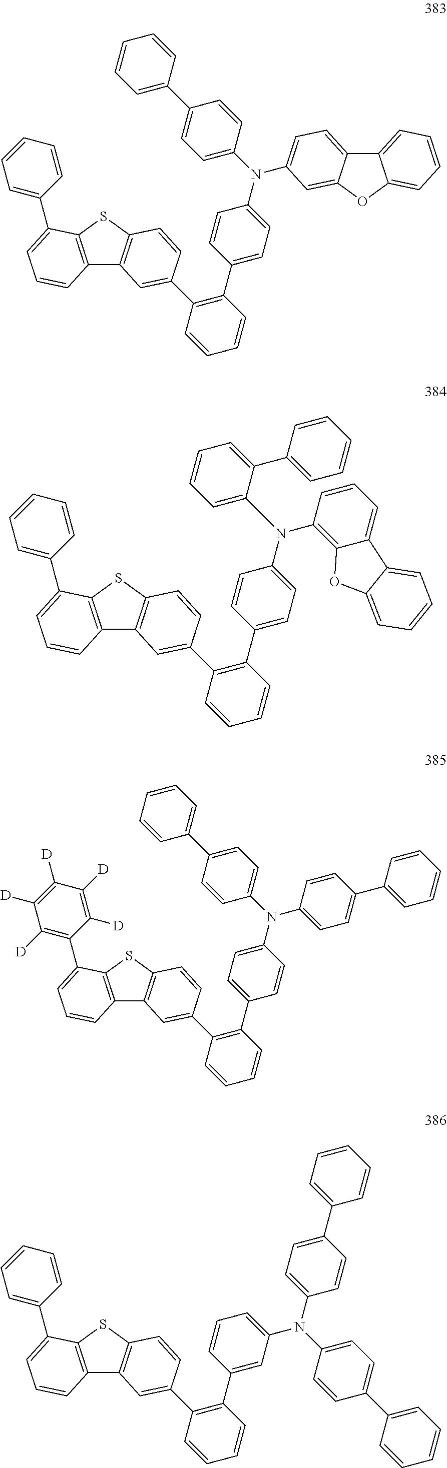 [SMILES: c1ccc(-c2ccc(N(c3ccc(-c4ccccc4-c4ccc5sc6c(-c7ccccc7)cccc6c5c4)cc3)c3ccc4c(c3)oc3ccccc34)cc2)cc1 ; c1ccc(-c2ccccc2N(c2ccc(-c3ccccc3-c3ccc4sc5c(-c6ccccc6)cccc5c4c3)cc2)c2cccc3c2oc2ccccc23)cc1 ; [2H]c1c([2H])c([2H])c(-c2cccc3c2sc2ccc(-c4ccccc4-c4ccc(N(c5ccc(-c6ccccc6)cc5)c5ccc(-c6ccccc6)cc5)cc4)cc23)c([2H])c1[2H] ; c1ccc(-c2ccc(N(c3ccc(-c4ccccc4)cc3)c3cccc(-c4ccccc4-c4ccc5sc6c(-c7ccccc7)cccc6c5c4)c3)cc2)cc1]

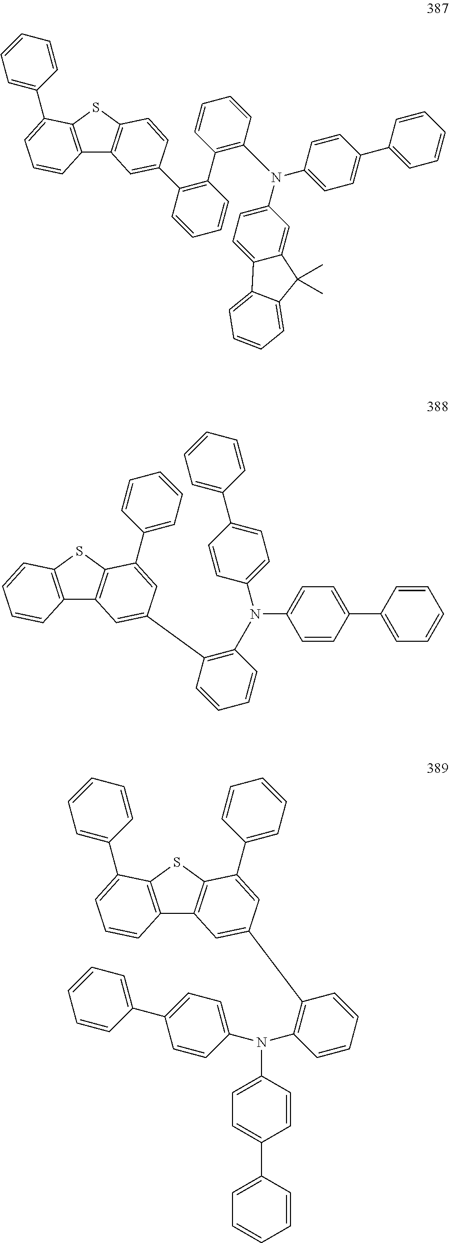 [SMILES: CC1(C)c2ccccc2-c2ccc(N(c3ccc(-c4ccccc4)cc3)c3ccccc3-c3ccccc3-c3ccc4sc5c(-c6ccccc6)cccc5c4c3)cc21 ; c1ccc(-c2ccc(N(c3ccc(-c4ccccc4)cc3)c3ccccc3-c3cc(-c4ccccc4)c4sc5ccccc5c4c3)cc2)cc1 ; c1ccc(-c2ccc(N(c3ccc(-c4ccccc4)cc3)c3ccccc3-c3cc(-c4ccccc4)c4sc5c(-c6ccccc6)cccc5c4c3)cc2)cc1]

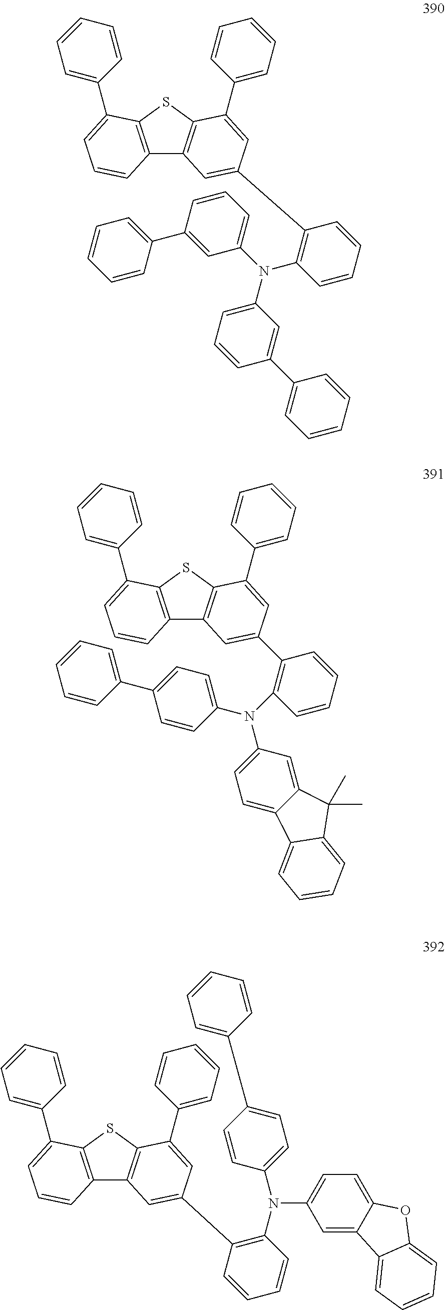 [SMILES: c1ccc(-c2cccc(N(c3cccc(-c4ccccc4)c3)c3ccccc3-c3cc(-c4ccccc4)c4sc5c(-c6ccccc6)cccc5c4c3)c2)cc1 ; CC1(C)c2ccccc2-c2ccc(N(c3ccc(-c4ccccc4)cc3)c3ccccc3-c3cc(-c4ccccc4)c4sc5c(-c6ccccc6)cccc5c4c3)cc21 ; c1ccc(-c2ccc(N(c3ccc4oc5ccccc5c4c3)c3ccccc3-c3cc(-c4ccccc4)c4sc5c(-c6ccccc6)cccc5c4c3)cc2)cc1]

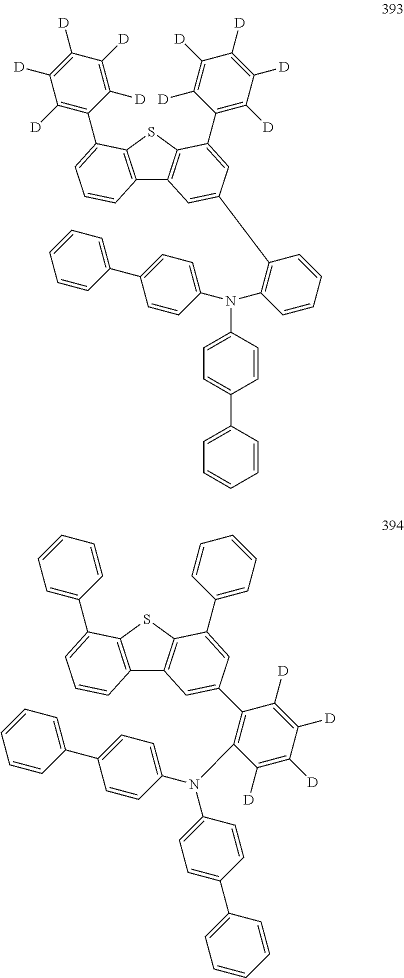 [SMILES: [2H]c1c([2H])c([2H])c(-c2cccc3c2sc2c(-c4c([2H])c([2H])c([2H])c([2H])c4[2H])cc(-c4ccccc4N(c4ccc(-c5ccccc5)cc4)c4ccc(-c5ccccc5)cc4)cc23)c([2H])c1[2H] ; [2H]c1c([2H])c([2H])c(N(c2ccc(-c3ccccc3)cc2)c2ccc(-c3ccccc3)cc2)c(-c2cc(-c3ccccc3)c3sc4c(-c5ccccc5)cccc4c3c2)c1[2H]]

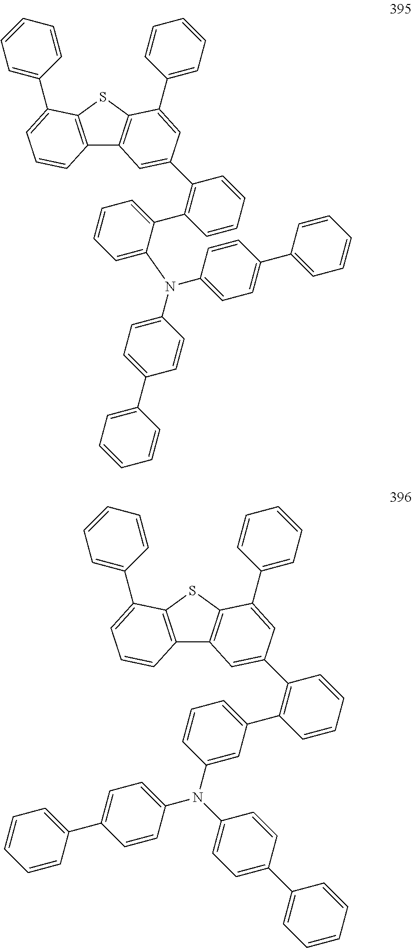

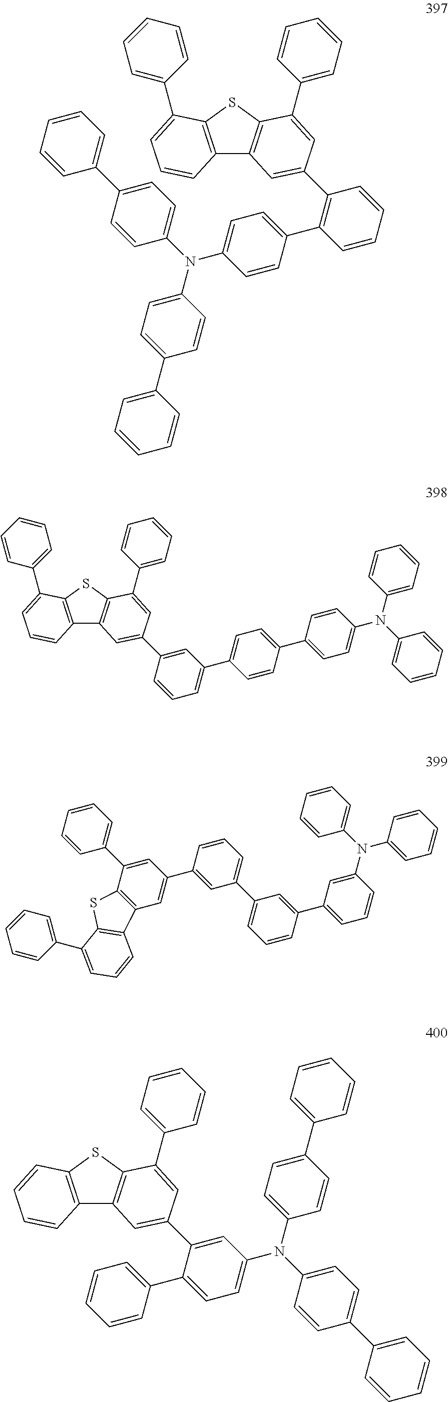 [SMILES: c1ccc(-c2ccc(N(c3ccc(-c4ccccc4)cc3)c3ccc(-c4ccccc4-c4cc(-c5ccccc5)c5sc6c(-c7ccccc7)cccc6c5c4)cc3)cc2)cc1 ; c1ccc(-c2cccc3c2sc2c(-c4ccccc4)cc(-c4cccc(-c5ccc(-c6ccc(N(c7ccccc7)c7ccccc7)cc6)cc5)c4)cc23)cc1 ; c1ccc(-c2cccc3c2sc2c(-c4ccccc4)cc(-c4cccc(-c5cccc(-c6cccc(N(c7ccccc7)c7ccccc7)c6)c5)c4)cc23)cc1 ; c1ccc(-c2ccc(N(c3ccc(-c4ccccc4)cc3)c3ccc(-c4ccccc4)c(-c4cc(-c5ccccc5)c5sc6ccccc6c5c4)c3)cc2)cc1]

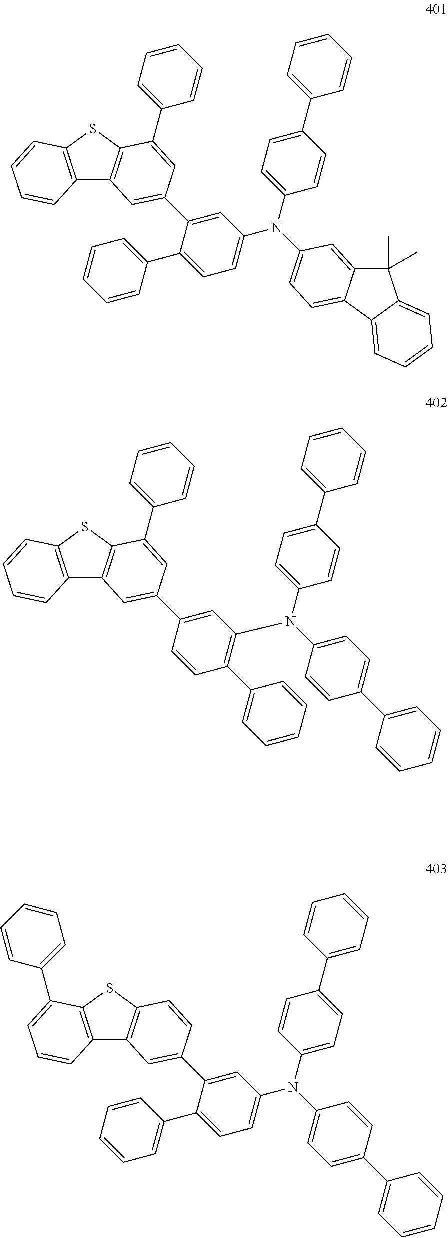 [SMILES: CC1(C)c2ccccc2-c2ccc(N(c3ccc(-c4ccccc4)cc3)c3ccc(-c4ccccc4)c(-c4cc(-c5ccccc5)c5sc6ccccc6c5c4)c3)cc21 ; c1ccc(-c2ccc(N(c3ccc(-c4ccccc4)cc3)c3cc(-c4cc(-c5ccccc5)c5sc6ccccc6c5c4)ccc3-c3ccccc3)cc2)cc1 ; c1ccc(-c2ccc(N(c3ccc(-c4ccccc4)cc3)c3ccc(-c4ccccc4)c(-c4ccc5sc6c(-c7ccccc7)cccc6c5c4)c3)cc2)cc1]

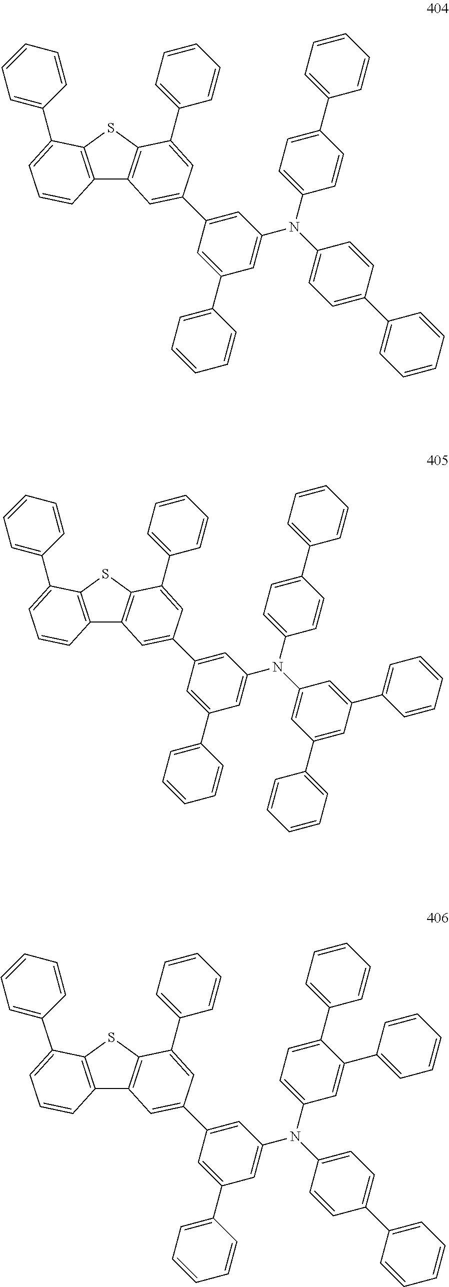 [SMILES: c1ccc(-c2ccc(N(c3ccc(-c4ccccc4)cc3)c3cc(-c4ccccc4)cc(-c4cc(-c5ccccc5)c5sc6c(-c7ccccc7)cccc6c5c4)c3)cc2)cc1 ; c1ccc(-c2ccc(N(c3cc(-c4ccccc4)cc(-c4ccccc4)c3)c3cc(-c4ccccc4)cc(-c4cc(-c5ccccc5)c5sc6c(-c7ccccc7)cccc6c5c4)c3)cc2)cc1 ; c1ccc(-c2ccc(N(c3cc(-c4ccccc4)cc(-c4cc(-c5ccccc5)c5sc6c(-c7ccccc7)cccc6c5c4)c3)c3ccc(-c4ccccc4)c(-c4ccccc4)c3)cc2)cc1]

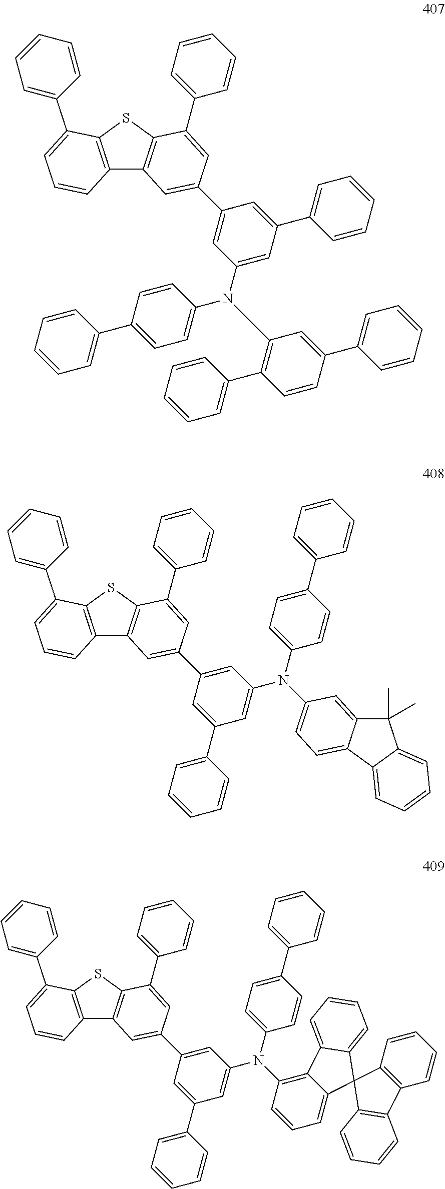 [SMILES: c1ccc(-c2ccc(N(c3cc(-c4ccccc4)cc(-c4cc(-c5ccccc5)c5sc6c(-c7ccccc7)cccc6c5c4)c3)c3cc(-c4ccccc4)ccc3-c3ccccc3)cc2)cc1 ; CC1(C)c2ccccc2-c2ccc(N(c3ccc(-c4ccccc4)cc3)c3cc(-c4ccccc4)cc(-c4cc(-c5ccccc5)c5sc6c(-c7ccccc7)cccc6c5c4)c3)cc21 ; c1ccc(-c2ccc(N(c3cc(-c4ccccc4)cc(-c4cc(-c5ccccc5)c5sc6c(-c7ccccc7)cccc6c5c4)c3)c3cccc4c3-c3ccccc3C43c4ccccc4-c4ccccc43)cc2)cc1]

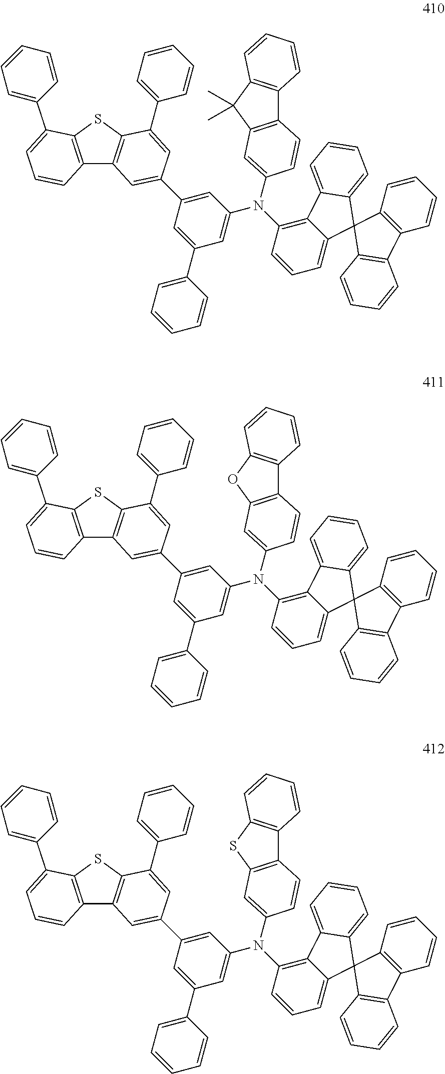 [SMILES: CC1(C)c2ccccc2-c2ccc(N(c3cc(-c4ccccc4)cc(-c4cc(-c5ccccc5)c5sc6c(-c7ccccc7)cccc6c5c4)c3)c3cccc4c3-c3ccccc3C43c4ccccc4-c4ccccc43)cc21 ; c1ccc(-c2cc(-c3cc(-c4ccccc4)c4sc5c(-c6ccccc6)cccc5c4c3)cc(N(c3ccc4c(c3)oc3ccccc34)c3cccc4c3-c3ccccc3C43c4ccccc4-c4ccccc43)c2)cc1 ; c1ccc(-c2cc(-c3cc(-c4ccccc4)c4sc5c(-c6ccccc6)cccc5c4c3)cc(N(c3ccc4c(c3)sc3ccccc34)c3cccc4c3-c3ccccc3C43c4ccccc4-c4ccccc43)c2)cc1]

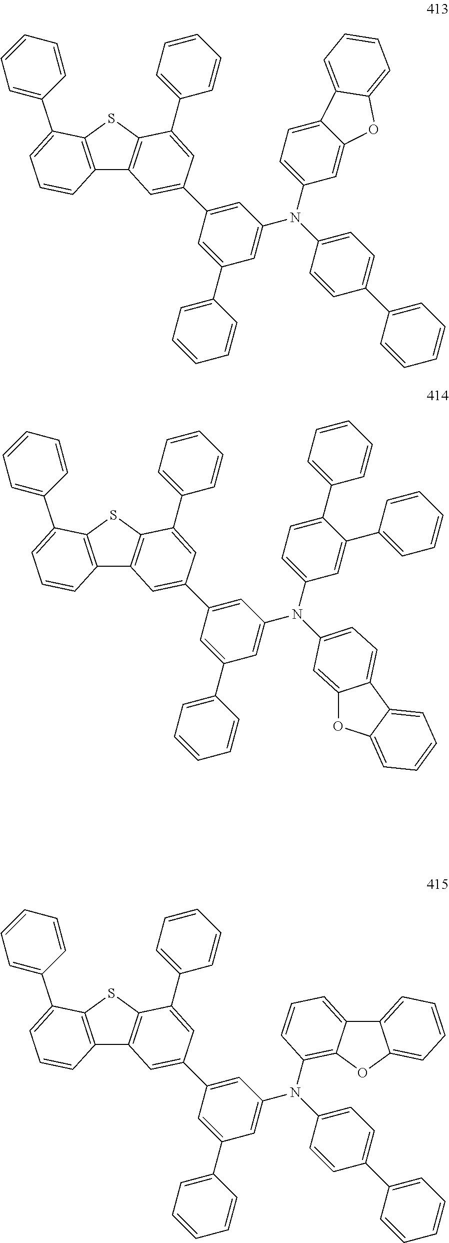 [SMILES: c1ccc(-c2ccc(N(c3cc(-c4ccccc4)cc(-c4cc(-c5ccccc5)c5sc6c(-c7ccccc7)cccc6c5c4)c3)c3ccc4c(c3)oc3ccccc34)cc2)cc1 ; c1ccc(-c2cc(-c3cc(-c4ccccc4)c4sc5c(-c6ccccc6)cccc5c4c3)cc(N(c3ccc(-c4ccccc4)c(-c4ccccc4)c3)c3ccc4c(c3)oc3ccccc34)c2)cc1 ; c1ccc(-c2ccc(N(c3cc(-c4ccccc4)cc(-c4cc(-c5ccccc5)c5sc6c(-c7ccccc7)cccc6c5c4)c3)c3cccc4c3oc3ccccc34)cc2)cc1]

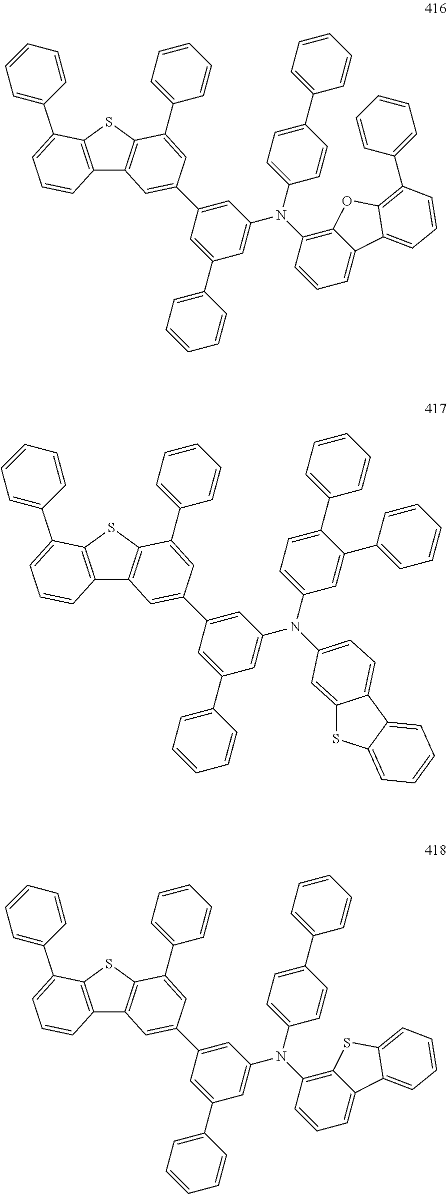 [SMILES: c1ccc(-c2ccc(N(c3cc(-c4ccccc4)cc(-c4cc(-c5ccccc5)c5sc6c(-c7ccccc7)cccc6c5c4)c3)c3cccc4c3oc3c(-c5ccccc5)cccc34)cc2)cc1 ; c1ccc(-c2cc(-c3cc(-c4ccccc4)c4sc5c(-c6ccccc6)cccc5c4c3)cc(N(c3ccc(-c4ccccc4)c(-c4ccccc4)c3)c3ccc4c(c3)sc3ccccc34)c2)cc1 ; c1ccc(-c2ccc(N(c3cc(-c4ccccc4)cc(-c4cc(-c5ccccc5)c5sc6c(-c7ccccc7)cccc6c5c4)c3)c3cccc4c3sc3ccccc34)cc2)cc1]

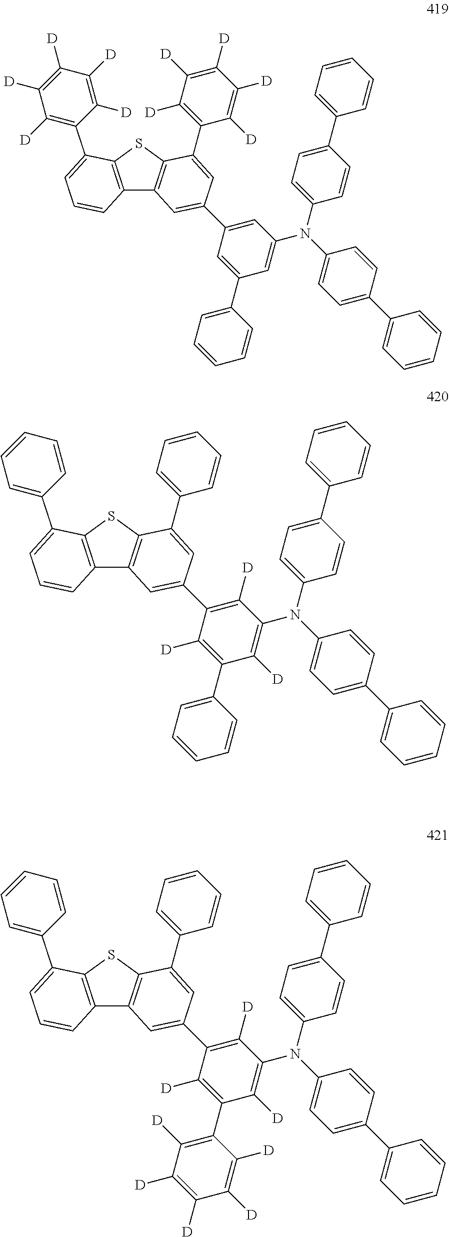 [SMILES: [2H]c1c([2H])c([2H])c(-c2cccc3c2sc2c(-c4c([2H])c([2H])c([2H])c([2H])c4[2H])cc(-c4cc(-c5ccccc5)cc(N(c5ccc(-c6ccccc6)cc5)c5ccc(-c6ccccc6)cc5)c4)cc23)c([2H])c1[2H] ; [2H]c1c(-c2ccccc2)c([2H])c(N(c2ccc(-c3ccccc3)cc2)c2ccc(-c3ccccc3)cc2)c([2H])c1-c1cc(-c2ccccc2)c2sc3c(-c4ccccc4)cccc3c2c1 ; [2H]c1c([2H])c([2H])c(-c2c([2H])c(-c3cc(-c4ccccc4)c4sc5c(-c6ccccc6)cccc5c4c3)c([2H])c(N(c3ccc(-c4ccccc4)cc3)c3ccc(-c4ccccc4)cc3)c2[2H])c([2H])c1[2H]]

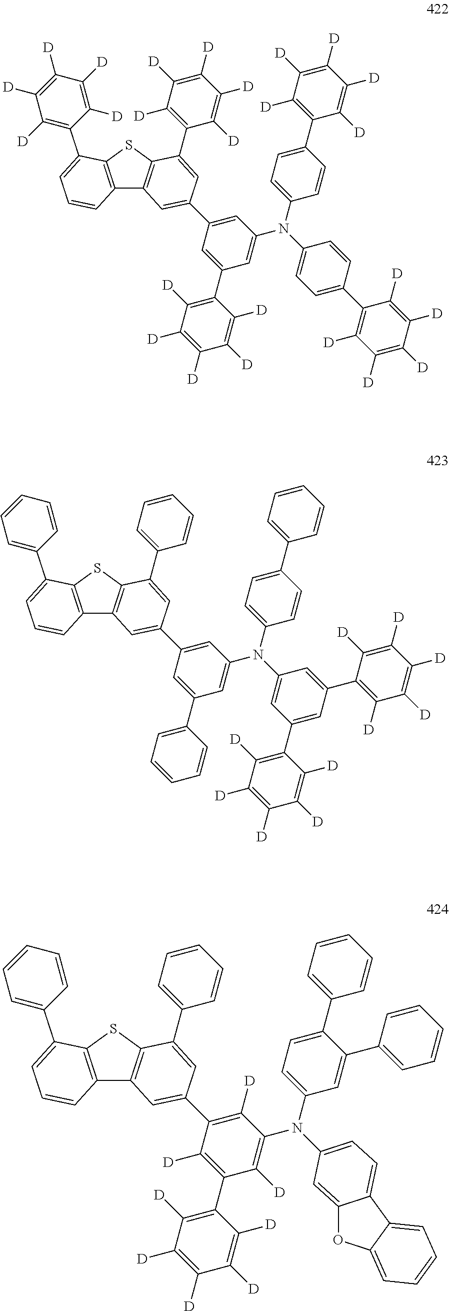

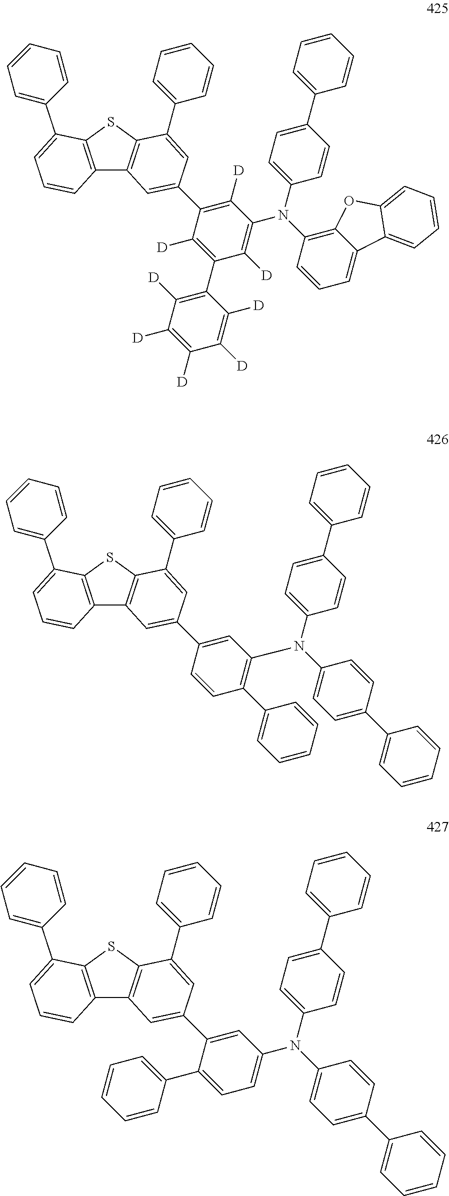 [SMILES: [2H]c1c([2H])c([2H])c(-c2c([2H])c(-c3cc(-c4ccccc4)c4sc5c(-c6ccccc6)cccc5c4c3)c([2H])c(N(c3ccc(-c4ccccc4)cc3)c3cccc4c3oc3ccccc34)c2[2H])c([2H])c1[2H] ; c1ccc(-c2ccc(N(c3ccc(-c4ccccc4)cc3)c3cc(-c4cc(-c5ccccc5)c5sc6c(-c7ccccc7)cccc6c5c4)ccc3-c3ccccc3)cc2)cc1 ; c1ccc(-c2ccc(N(c3ccc(-c4ccccc4)cc3)c3ccc(-c4ccccc4)c(-c4cc(-c5ccccc5)c5sc6c(-c7ccccc7)cccc6c5c4)c3)cc2)cc1]

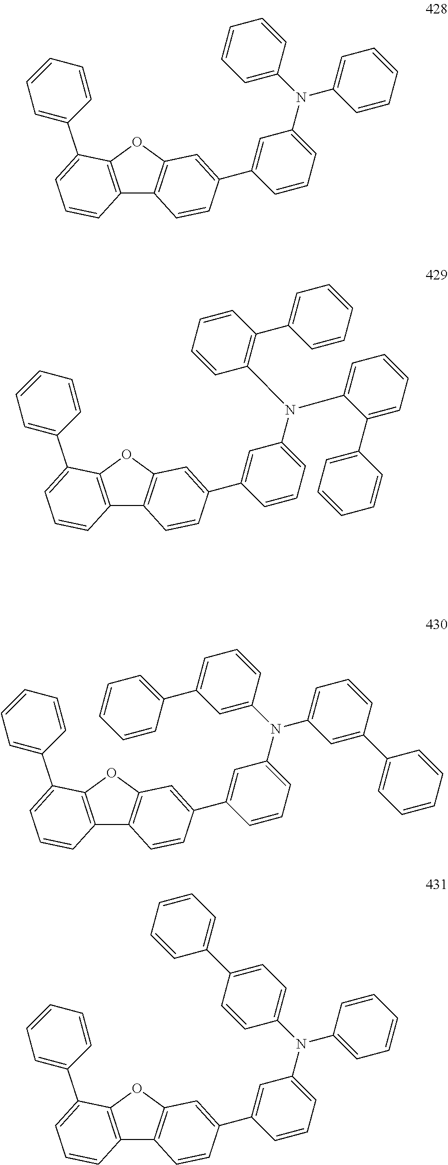 [SMILES: c1ccc(-c2cccc3c2oc2cc(-c4cccc(N(c5ccccc5)c5ccccc5)c4)ccc23)cc1 ; c1ccc(-c2ccccc2N(c2cccc(-c3ccc4c(c3)oc3c(-c5ccccc5)cccc34)c2)c2ccccc2-c2ccccc2)cc1 ; c1ccc(-c2cccc(N(c3cccc(-c4ccccc4)c3)c3cccc(-c4ccc5c(c4)oc4c(-c6ccccc6)cccc45)c3)c2)cc1 ; c1ccc(-c2ccc(N(c3ccccc3)c3cccc(-c4ccc5c(c4)oc4c(-c6ccccc6)cccc45)c3)cc2)cc1]

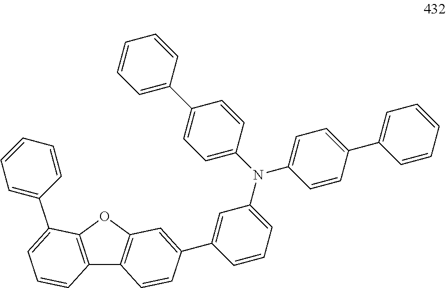 [SMILES: c1ccc(-c2ccc(N(c3ccc(-c4ccccc4)cc3)c3cccc(-c4ccc5c(c4)oc4c(-c6ccccc6)cccc45)c3)cc2)cc1]

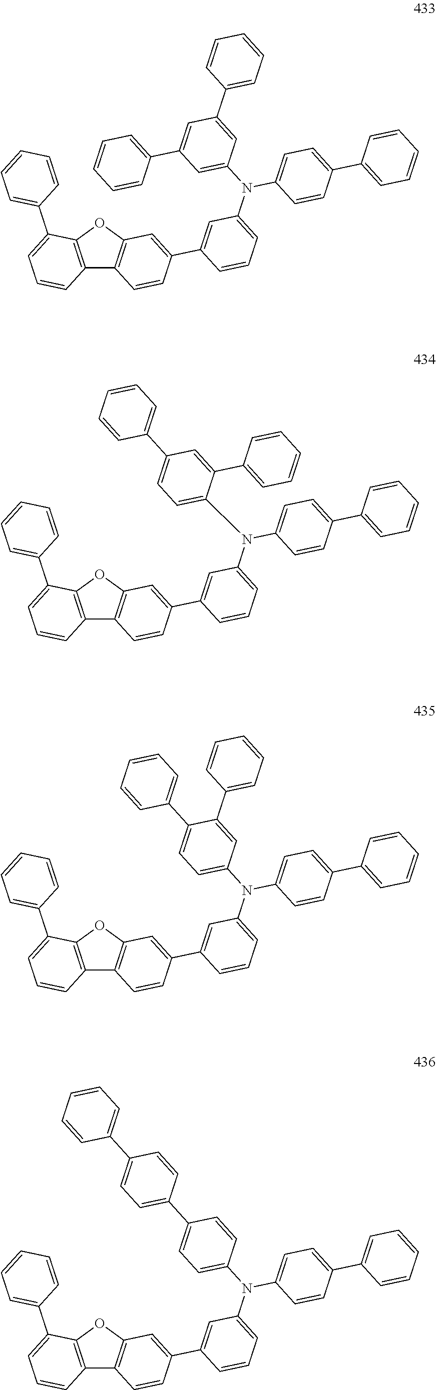 [SMILES: c1ccc(-c2ccc(N(c3cccc(-c4ccc5c(c4)oc4c(-c6ccccc6)cccc45)c3)c3cc(-c4ccccc4)cc(-c4ccccc4)c3)cc2)cc1 ; c1ccc(-c2ccc(N(c3cccc(-c4ccc5c(c4)oc4c(-c6ccccc6)cccc45)c3)c3ccc(-c4ccccc4)cc3-c3ccccc3)cc2)cc1 ; c1ccc(-c2ccc(N(c3cccc(-c4ccc5c(c4)oc4c(-c6ccccc6)cccc45)c3)c3ccc(-c4ccccc4)c(-c4ccccc4)c3)cc2)cc1 ; c1ccc(-c2ccc(-c3ccc(N(c4ccc(-c5ccccc5)cc4)c4cccc(-c5ccc6c(c5)oc5c(-c7ccccc7)cccc56)c4)cc3)cc2)cc1]

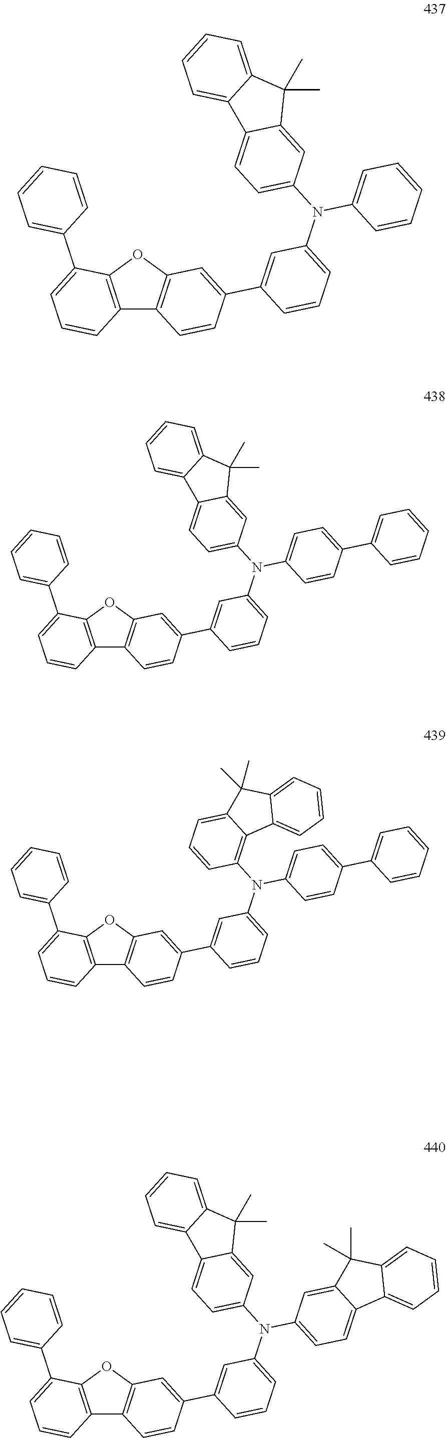 [SMILES: CC1(C)c2ccccc2-c2ccc(N(c3ccccc3)c3cccc(-c4ccc5c(c4)oc4c(-c6ccccc6)cccc45)c3)cc21 ; CC1(C)c2ccccc2-c2ccc(N(c3ccc(-c4ccccc4)cc3)c3cccc(-c4ccc5c(c4)oc4c(-c6ccccc6)cccc45)c3)cc21 ; CC1(C)c2ccccc2-c2c(N(c3ccc(-c4ccccc4)cc3)c3cccc(-c4ccc5c(c4)oc4c(-c6ccccc6)cccc45)c3)cccc21 ; CC1(C)c2ccccc2-c2ccc(N(c3cccc(-c4ccc5c(c4)oc4c(-c6ccccc6)cccc45)c3)c3ccc4c(c3)C(C)(C)c3ccccc3-4)cc21]

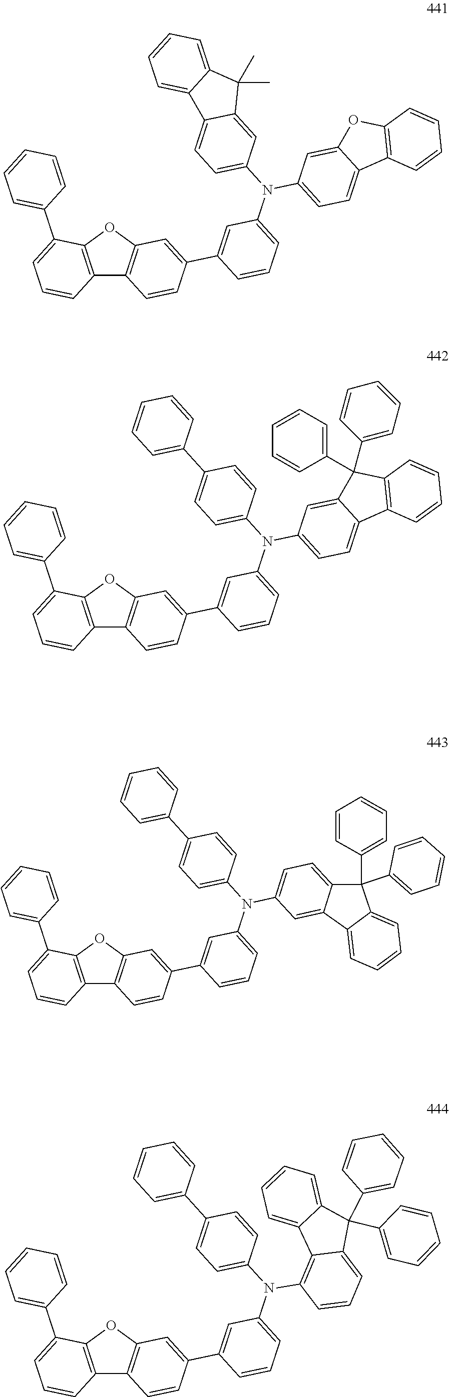 [SMILES: CC1(C)c2ccccc2-c2ccc(N(c3cccc(-c4ccc5c(c4)oc4c(-c6ccccc6)cccc45)c3)c3ccc4c(c3)oc3ccccc34)cc21 ; c1ccc(-c2ccc(N(c3cccc(-c4ccc5c(c4)oc4c(-c6ccccc6)cccc45)c3)c3ccc4c(c3)C(c3ccccc3)(c3ccccc3)c3ccccc3-4)cc2)cc1 ; c1ccc(-c2ccc(N(c3cccc(-c4ccc5c(c4)oc4c(-c6ccccc6)cccc45)c3)c3ccc4c(c3)-c3ccccc3C4(c3ccccc3)c3ccccc3)cc2)cc1 ; c1ccc(-c2ccc(N(c3cccc(-c4ccc5c(c4)oc4c(-c6ccccc6)cccc45)c3)c3cccc4c3-c3ccccc3C4(c3ccccc3)c3ccccc3)cc2)cc1]

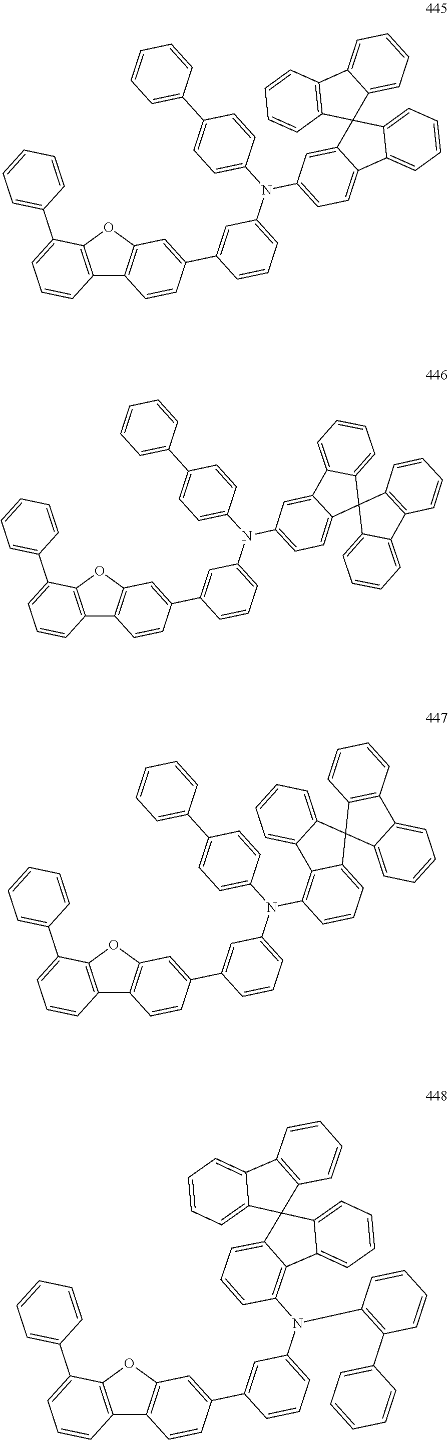 [SMILES: c1ccc(-c2ccc(N(c3cccc(-c4ccc5c(c4)oc4c(-c6ccccc6)cccc45)c3)c3ccc4c(c3)C3(c5ccccc5-c5ccccc53)c3ccccc3-4)cc2)cc1 ; c1ccc(-c2ccc(N(c3cccc(-c4ccc5c(c4)oc4c(-c6ccccc6)cccc45)c3)c3ccc4c(c3)-c3ccccc3C43c4ccccc4-c4ccccc43)cc2)cc1 ; c1ccc(-c2ccc(N(c3cccc(-c4ccc5c(c4)oc4c(-c6ccccc6)cccc45)c3)c3cccc4c3-c3ccccc3C43c4ccccc4-c4ccccc43)cc2)cc1 ; c1ccc(-c2ccccc2N(c2cccc(-c3ccc4c(c3)oc3c(-c5ccccc5)cccc34)c2)c2cccc3c2-c2ccccc2C32c3ccccc3-c3ccccc32)cc1]

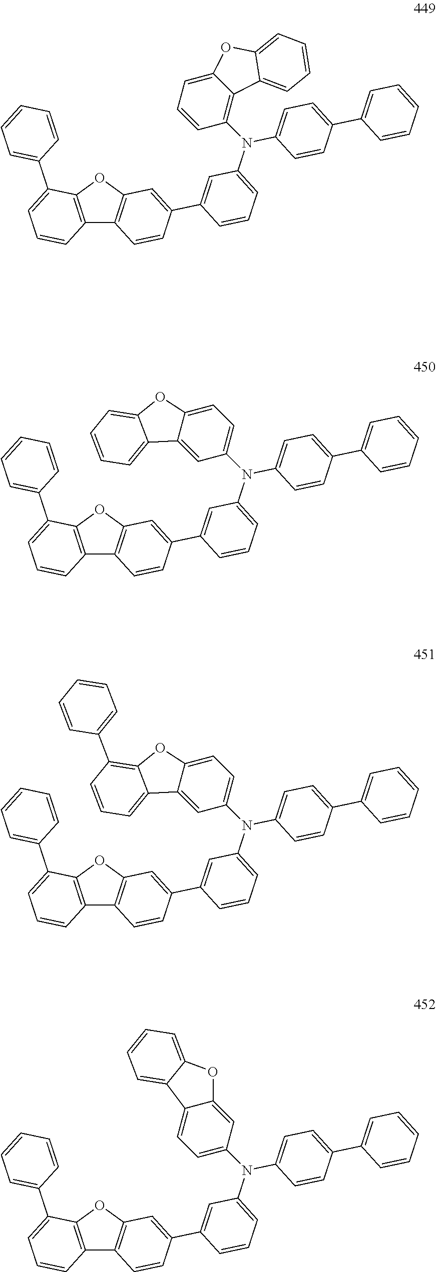 [SMILES: c1ccc(-c2ccc(N(c3cccc(-c4ccc5c(c4)oc4c(-c6ccccc6)cccc45)c3)c3cccc4oc5ccccc5c34)cc2)cc1 ; c1ccc(-c2ccc(N(c3cccc(-c4ccc5c(c4)oc4c(-c6ccccc6)cccc45)c3)c3ccc4oc5ccccc5c4c3)cc2)cc1 ; c1ccc(-c2ccc(N(c3cccc(-c4ccc5c(c4)oc4c(-c6ccccc6)cccc45)c3)c3ccc4oc5c(-c6ccccc6)cccc5c4c3)cc2)cc1 ; c1ccc(-c2ccc(N(c3cccc(-c4ccc5c(c4)oc4c(-c6ccccc6)cccc45)c3)c3ccc4c(c3)oc3ccccc34)cc2)cc1]

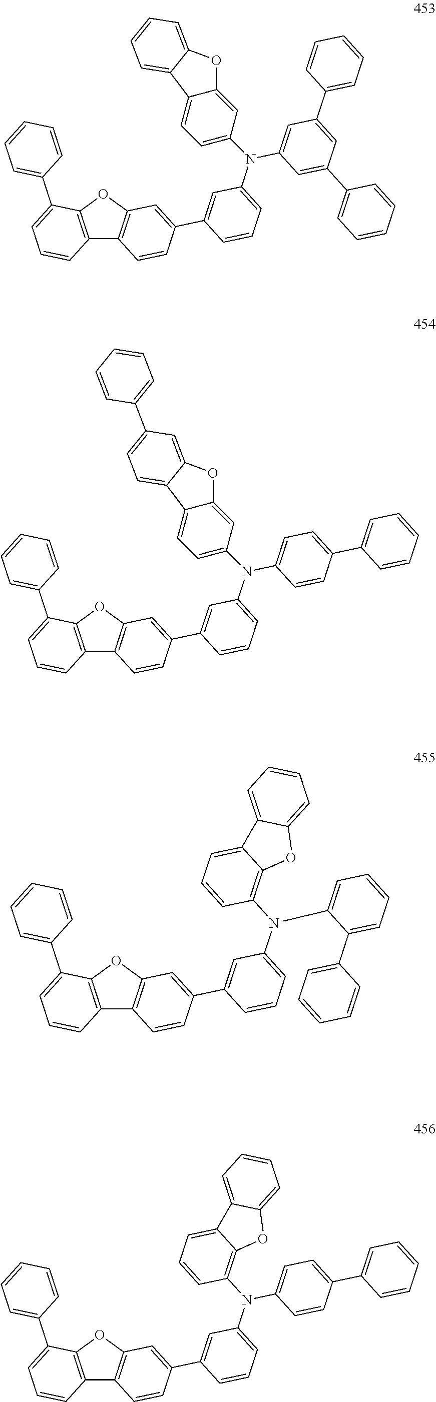 [SMILES: c1ccc(-c2cc(-c3ccccc3)cc(N(c3cccc(-c4ccc5c(c4)oc4c(-c6ccccc6)cccc45)c3)c3ccc4c(c3)oc3ccccc34)c2)cc1 ; c1ccc(-c2ccc(N(c3cccc(-c4ccc5c(c4)oc4c(-c6ccccc6)cccc45)c3)c3ccc4c(c3)oc3cc(-c5ccccc5)ccc34)cc2)cc1 ; c1ccc(-c2ccccc2N(c2cccc(-c3ccc4c(c3)oc3c(-c5ccccc5)cccc34)c2)c2cccc3c2oc2ccccc23)cc1 ; c1ccc(-c2ccc(N(c3cccc(-c4ccc5c(c4)oc4c(-c6ccccc6)cccc45)c3)c3cccc4c3oc3ccccc34)cc2)cc1]

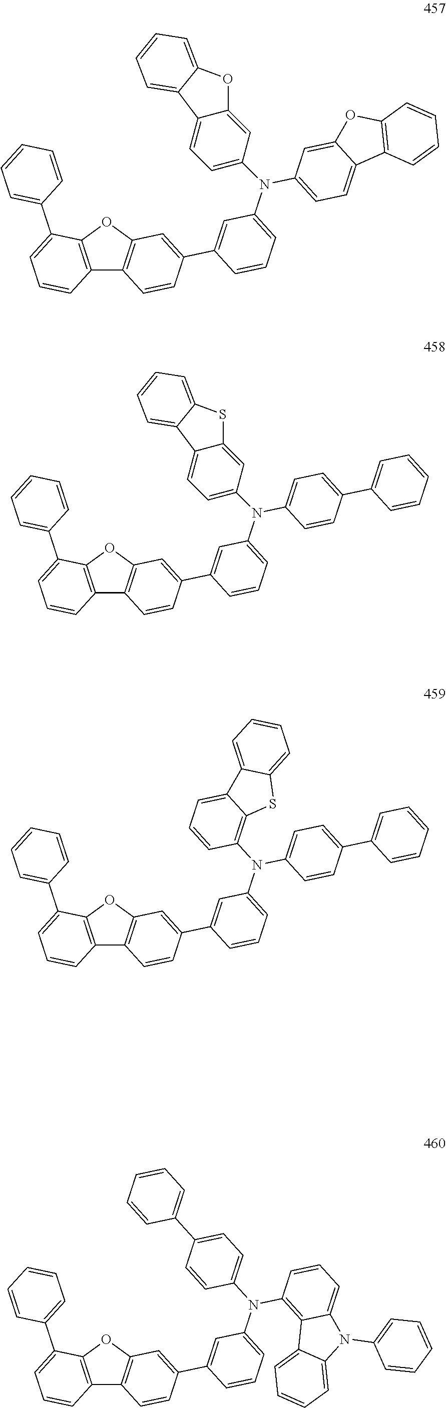 [SMILES: c1ccc(-c2cccc3c2oc2cc(-c4cccc(N(c5ccc6c(c5)oc5ccccc56)c5ccc6c(c5)oc5ccccc56)c4)ccc23)cc1 ; c1ccc(-c2ccc(N(c3cccc(-c4ccc5c(c4)oc4c(-c6ccccc6)cccc45)c3)c3ccc4c(c3)sc3ccccc34)cc2)cc1 ; c1ccc(-c2ccc(N(c3cccc(-c4ccc5c(c4)oc4c(-c6ccccc6)cccc45)c3)c3cccc4c3sc3ccccc34)cc2)cc1 ; c1ccc(-c2ccc(N(c3cccc(-c4ccc5c(c4)oc4c(-c6ccccc6)cccc45)c3)c3cccc4c3c3ccccc3n4-c3ccccc3)cc2)cc1]

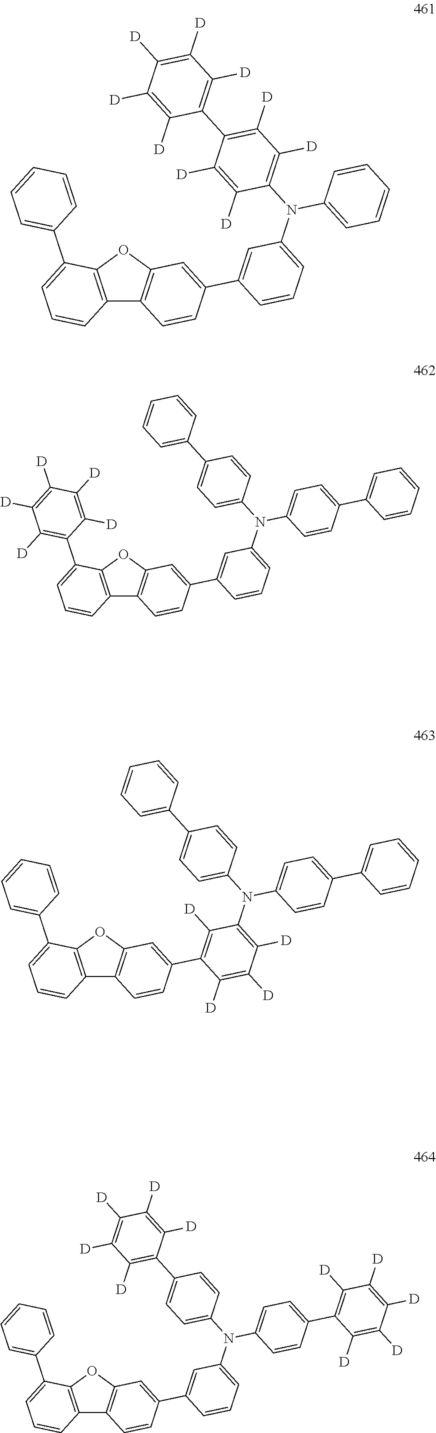 [SMILES: [2H]c1c([2H])c([2H])c(-c2c([2H])c([2H])c(N(c3ccccc3)c3cccc(-c4ccc5c(c4)oc4c(-c6ccccc6)cccc45)c3)c([2H])c2[2H])c([2H])c1[2H] ; [2H]c1c([2H])c([2H])c(-c2cccc3c2oc2cc(-c4cccc(N(c5ccc(-c6ccccc6)cc5)c5ccc(-c6ccccc6)cc5)c4)ccc23)c([2H])c1[2H] ; [2H]c1c([2H])c(-c2ccc3c(c2)oc2c(-c4ccccc4)cccc23)c([2H])c(N(c2ccc(-c3ccccc3)cc2)c2ccc(-c3ccccc3)cc2)c1[2H] ; [2H]c1c([2H])c([2H])c(-c2ccc(N(c3ccc(-c4c([2H])c([2H])c([2H])c([2H])c4[2H])cc3)c3cccc(-c4ccc5c(c4)oc4c(-c6ccccc6)cccc45)c3)cc2)c([2H])c1[2H]]

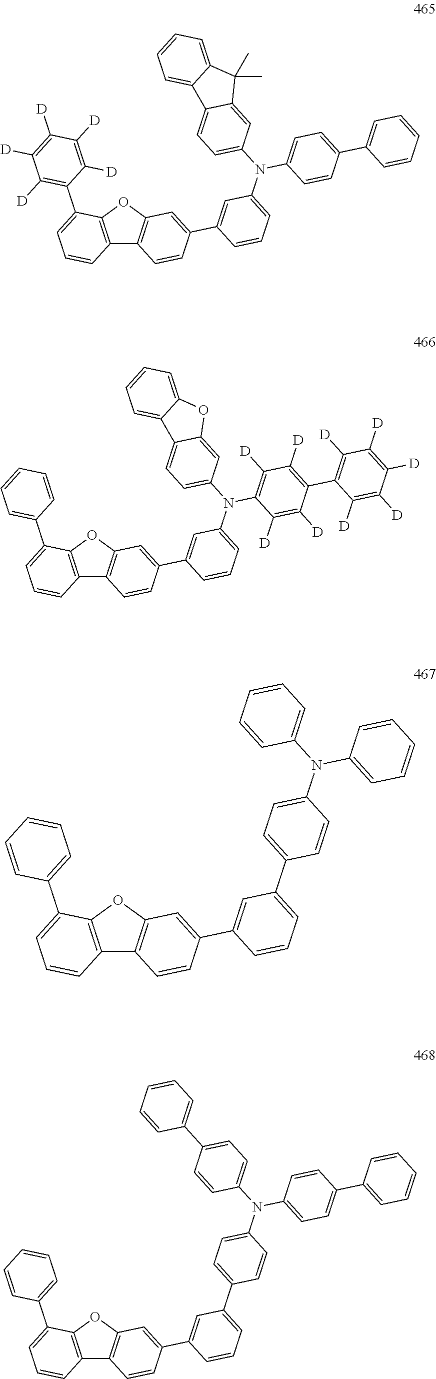 [SMILES: [2H]c1c([2H])c([2H])c(-c2cccc3c2oc2cc(-c4cccc(N(c5ccc(-c6ccccc6)cc5)c5ccc6c(c5)C(C)(C)c5ccccc5-6)c4)ccc23)c([2H])c1[2H] ; [2H]c1c([2H])c([2H])c(-c2c([2H])c([2H])c(N(c3cccc(-c4ccc5c(c4)oc4c(-c6ccccc6)cccc45)c3)c3ccc4c(c3)oc3ccccc34)c([2H])c2[2H])c([2H])c1[2H] ; c1ccc(-c2cccc3c2oc2cc(-c4cccc(-c5ccc(N(c6ccccc6)c6ccccc6)cc5)c4)ccc23)cc1 ; c1ccc(-c2ccc(N(c3ccc(-c4ccccc4)cc3)c3ccc(-c4cccc(-c5ccc6c(c5)oc5c(-c7ccccc7)cccc56)c4)cc3)cc2)cc1]

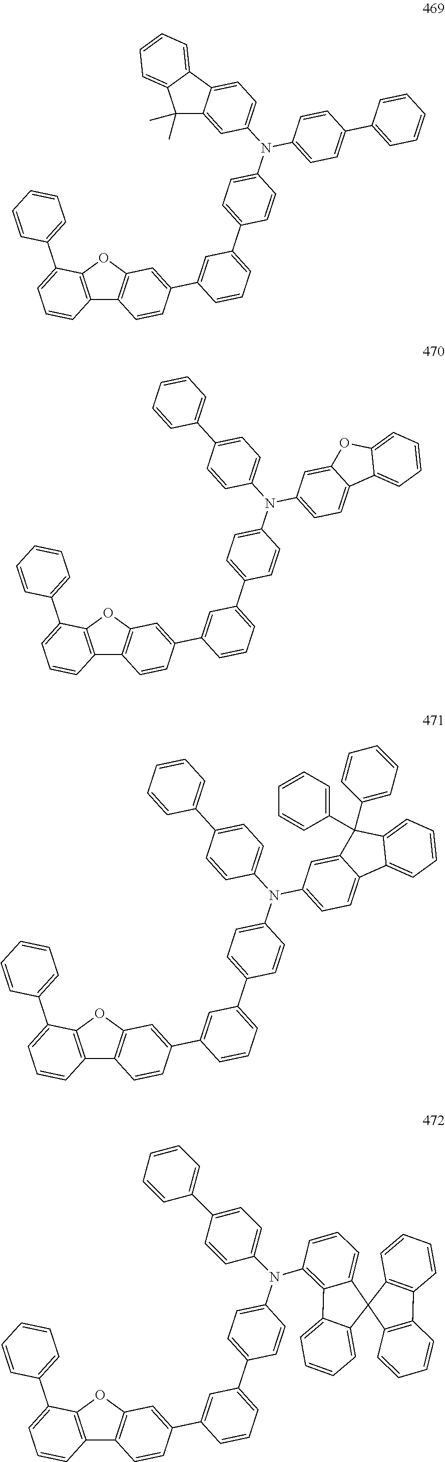 [SMILES: CC1(C)c2ccccc2-c2ccc(N(c3ccc(-c4ccccc4)cc3)c3ccc(-c4cccc(-c5ccc6c(c5)oc5c(-c7ccccc7)cccc56)c4)cc3)cc21 ; c1ccc(-c2ccc(N(c3ccc(-c4cccc(-c5ccc6c(c5)oc5c(-c7ccccc7)cccc56)c4)cc3)c3ccc4c(c3)oc3ccccc34)cc2)cc1 ; c1ccc(-c2ccc(N(c3ccc(-c4cccc(-c5ccc6c(c5)oc5c(-c7ccccc7)cccc56)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c(c5)oc5c(-c7ccccc7)cccc56)c4)cc3)c3cccc4c3-c3ccccc3C43c4ccccc4-c4ccccc43)cc2)cc1]

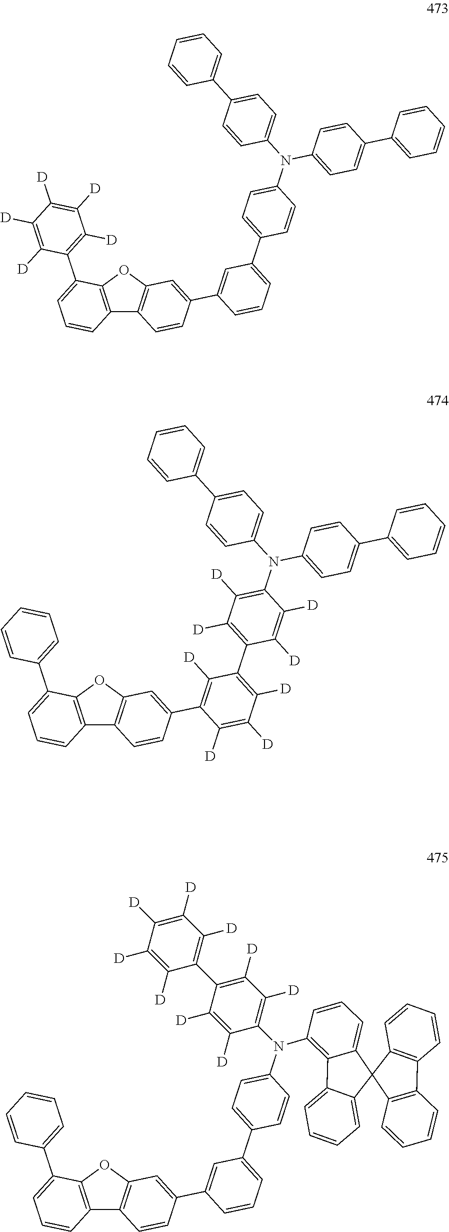 [SMILES: [2H]c1c([2H])c([2H])c(-c2cccc3c2oc2cc(-c4cccc(-c5ccc(N(c6ccc(-c7ccccc7)cc6)c6ccc(-c7ccccc7)cc6)cc5)c4)ccc23)c([2H])c1[2H] ; [2H]c1c([2H])c(-c2ccc3c(c2)oc2c(-c4ccccc4)cccc23)c([2H])c(-c2c([2H])c([2H])c(N(c3ccc(-c4ccccc4)cc3)c3ccc(-c4ccccc4)cc3)c([2H])c2[2H])c1[2H] ; [2H]c1c([2H])c([2H])c(-c2c([2H])c([2H])c(N(c3ccc(-c4cccc(-c5ccc6c(c5)oc5c(-c7ccccc7)cccc56)c4)cc3)c3cccc4c3-c3ccccc3C43c4ccccc4-c4ccccc43)c([2H])c2[2H])c([2H])c1[2H]]

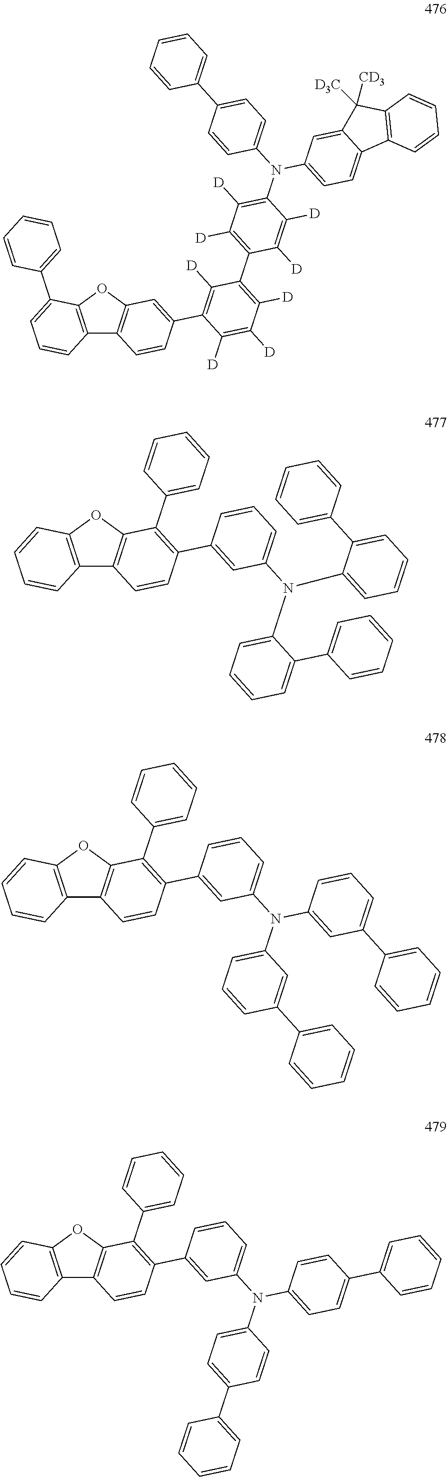 [SMILES: [2H]c1c([2H])c(-c2ccc3c(c2)oc2c(-c4ccccc4)cccc23)c([2H])c(-c2c([2H])c([2H])c(N(c3ccc(-c4ccccc4)cc3)c3ccc4c(c3)C(C([2H])([2H])[2H])(C([2H])([2H])[2H])c3ccccc3-4)c([2H])c2[2H])c1[2H] ; c1ccc(-c2ccccc2N(c2cccc(-c3ccc4c(oc5ccccc54)c3-c3ccccc3)c2)c2ccccc2-c2ccccc2)cc1 ; c1ccc(-c2cccc(N(c3cccc(-c4ccccc4)c3)c3cccc(-c4ccc5c(oc6ccccc65)c4-c4ccccc4)c3)c2)cc1 ; c1ccc(-c2ccc(N(c3ccc(-c4ccccc4)cc3)c3cccc(-c4ccc5c(oc6ccccc65)c4-c4ccccc4)c3)cc2)cc1]

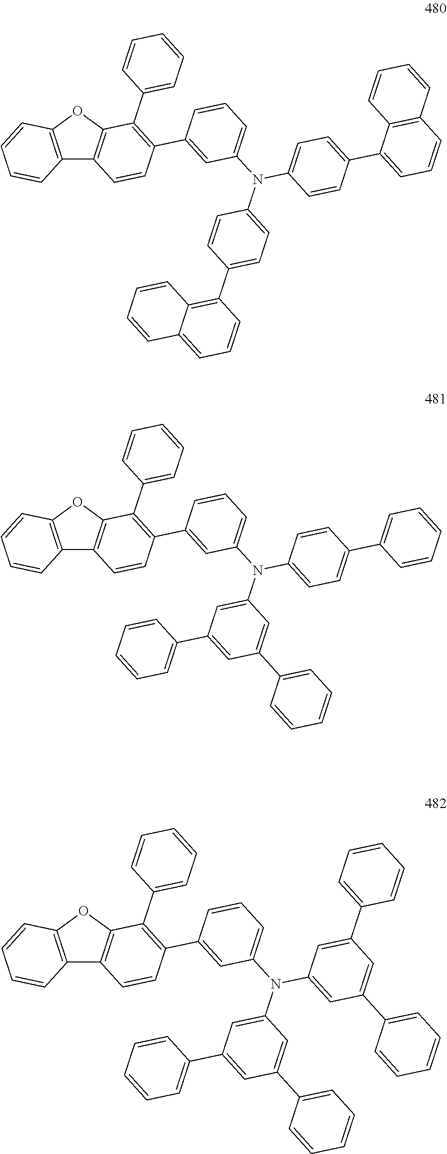 [SMILES: c1ccc(-c2c(-c3cccc(N(c4ccc(-c5cccc6ccccc56)cc4)c4ccc(-c5cccc6ccccc56)cc4)c3)ccc3c2oc2ccccc23)cc1 ; c1ccc(-c2ccc(N(c3cc(-c4ccccc4)cc(-c4ccccc4)c3)c3cccc(-c4ccc5c(oc6ccccc65)c4-c4ccccc4)c3)cc2)cc1 ; c1ccc(-c2cc(-c3ccccc3)cc(N(c3cc(-c4ccccc4)cc(-c4ccccc4)c3)c3cccc(-c4ccc5c(oc6ccccc65)c4-c4ccccc4)c3)c2)cc1]

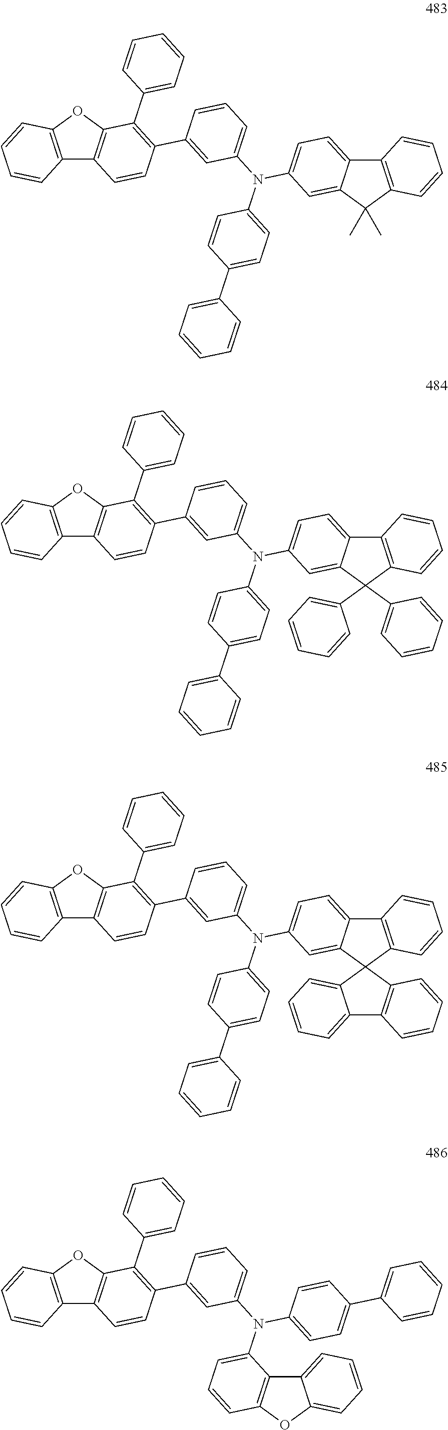 [SMILES: CC1(C)c2ccccc2-c2ccc(N(c3ccc(-c4ccccc4)cc3)c3cccc(-c4ccc5c(oc6ccccc65)c4-c4ccccc4)c3)cc21 ; c1ccc(-c2ccc(N(c3cccc(-c4ccc5c(oc6ccccc65)c4-c4ccccc4)c3)c3ccc4c(c3)C(c3ccccc3)(c3ccccc3)c3ccccc3-4)cc2)cc1 ; c1ccc(-c2ccc(N(c3cccc(-c4ccc5c(oc6ccccc65)c4-c4ccccc4)c3)c3ccc4c(c3)C3(c5ccccc5-c5ccccc53)c3ccccc3-4)cc2)cc1 ; c1ccc(-c2ccc(N(c3cccc(-c4ccc5c(oc6ccccc65)c4-c4ccccc4)c3)c3cccc4oc5ccccc5c34)cc2)cc1]

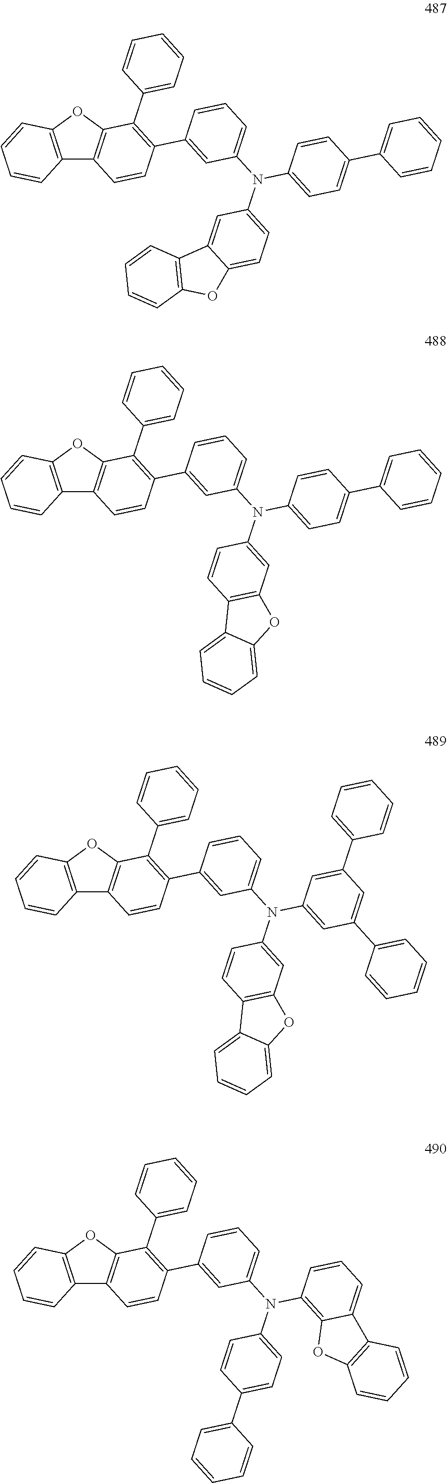 [SMILES: c1ccc(-c2ccc(N(c3cccc(-c4ccc5c(oc6ccccc65)c4-c4ccccc4)c3)c3ccc4oc5ccccc5c4c3)cc2)cc1 ; c1ccc(-c2ccc(N(c3cccc(-c4ccc5c(oc6ccccc65)c4-c4ccccc4)c3)c3ccc4c(c3)oc3ccccc34)cc2)cc1 ; c1ccc(-c2cc(-c3ccccc3)cc(N(c3cccc(-c4ccc5c(oc6ccccc65)c4-c4ccccc4)c3)c3ccc4c(c3)oc3ccccc34)c2)cc1 ; c1ccc(-c2ccc(N(c3cccc(-c4ccc5c(oc6ccccc65)c4-c4ccccc4)c3)c3cccc4c3oc3ccccc34)cc2)cc1]

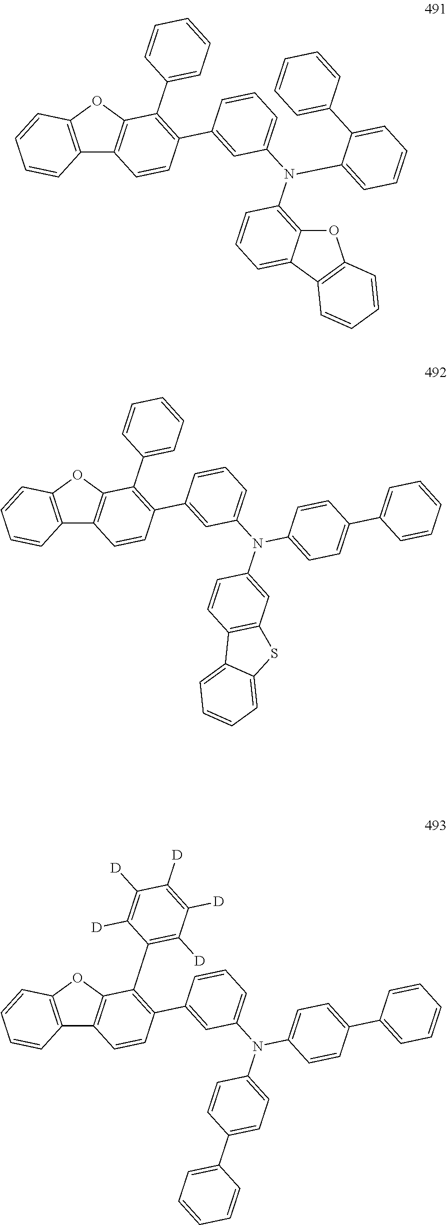 [SMILES: c1ccc(-c2ccccc2N(c2cccc(-c3ccc4c(oc5ccccc54)c3-c3ccccc3)c2)c2cccc3c2oc2ccccc23)cc1 ; c1ccc(-c2ccc(N(c3cccc(-c4ccc5c(oc6ccccc65)c4-c4ccccc4)c3)c3ccc4c(c3)sc3ccccc34)cc2)cc1 ; [2H]c1c([2H])c([2H])c(-c2c(-c3cccc(N(c4ccc(-c5ccccc5)cc4)c4ccc(-c5ccccc5)cc4)c3)ccc3c2oc2ccccc23)c([2H])c1[2H]]

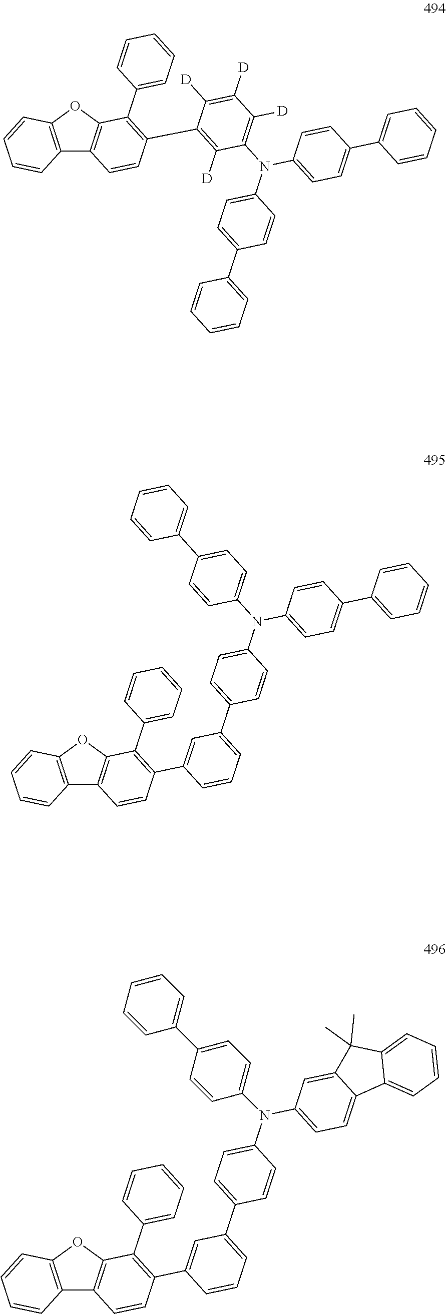 [SMILES: [2H]c1c([2H])c(-c2ccc3c(oc4ccccc43)c2-c2ccccc2)c([2H])c(N(c2ccc(-c3ccccc3)cc2)c2ccc(-c3ccccc3)cc2)c1[2H] ; c1ccc(-c2ccc(N(c3ccc(-c4ccccc4)cc3)c3ccc(-c4cccc(-c5ccc6c(oc7ccccc76)c5-c5ccccc5)c4)cc3)cc2)cc1 ; CC1(C)c2ccccc2-c2ccc(N(c3ccc(-c4ccccc4)cc3)c3ccc(-c4cccc(-c5ccc6c(oc7ccccc76)c5-c5ccccc5)c4)cc3)cc21]

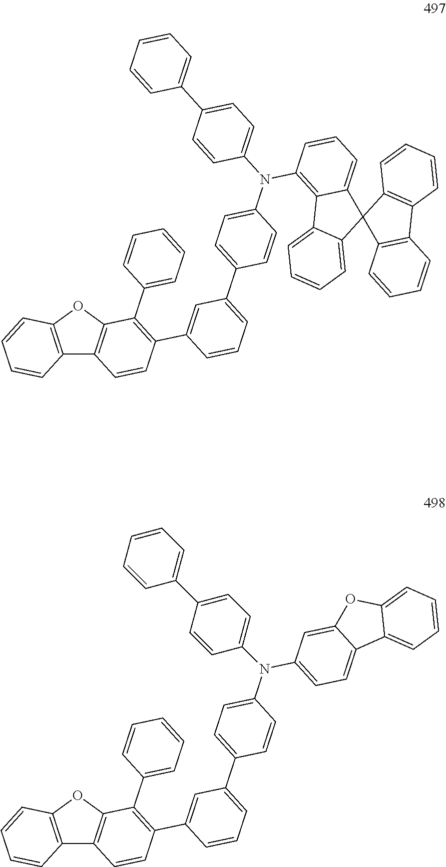 [SMILES: c1ccc(-c2ccc(N(c3ccc(-c4cccc(-c5ccc6c(oc7ccccc76)c5-c5ccccc5)c4)cc3)c3cccc4c3-c3ccccc3C43c4ccccc4-c4ccccc43)cc2)cc1 ; c1ccc(-c2ccc(N(c3ccc(-c4cccc(-c5ccc6c(oc7ccccc76)c5-c5ccccc5)c4)cc3)c3ccc4c(c3)oc3ccccc34)cc2)cc1]

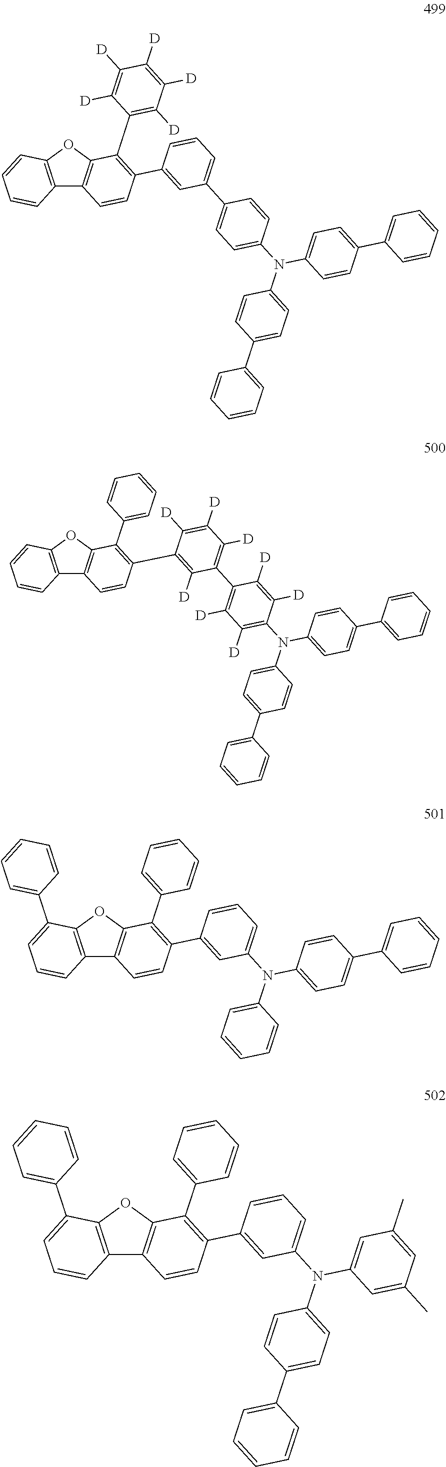 [SMILES: [2H]c1c([2H])c([2H])c(-c2c(-c3cccc(-c4ccc(N(c5ccc(-c6ccccc6)cc5)c5ccc(-c6ccccc6)cc5)cc4)c3)ccc3c2oc2ccccc23)c([2H])c1[2H] ; [2H]c1c([2H])c(-c2ccc3c(oc4ccccc43)c2-c2ccccc2)c([2H])c(-c2c([2H])c([2H])c(N(c3ccc(-c4ccccc4)cc3)c3ccc(-c4ccccc4)cc3)c([2H])c2[2H])c1[2H] ; c1ccc(-c2ccc(N(c3ccccc3)c3cccc(-c4ccc5c(oc6c(-c7ccccc7)cccc65)c4-c4ccccc4)c3)cc2)cc1 ; Cc1cc(C)cc(N(c2ccc(-c3ccccc3)cc2)c2cccc(-c3ccc4c(oc5c(-c6ccccc6)cccc54)c3-c3ccccc3)c2)c1]

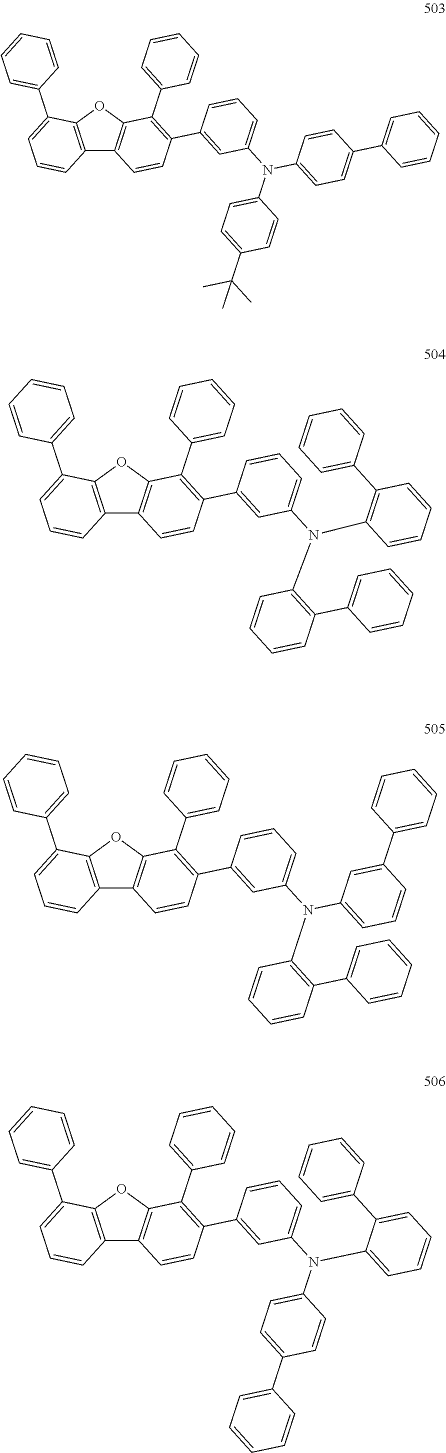 [SMILES: CC(C)(C)c1ccc(N(c2ccc(-c3ccccc3)cc2)c2cccc(-c3ccc4c(oc5c(-c6ccccc6)cccc54)c3-c3ccccc3)c2)cc1 ; c1ccc(-c2ccccc2N(c2cccc(-c3ccc4c(oc5c(-c6ccccc6)cccc54)c3-c3ccccc3)c2)c2ccccc2-c2ccccc2)cc1 ; c1ccc(-c2cccc(N(c3cccc(-c4ccc5c(oc6c(-c7ccccc7)cccc65)c4-c4ccccc4)c3)c3ccccc3-c3ccccc3)c2)cc1 ; c1ccc(-c2ccc(N(c3cccc(-c4ccc5c(oc6c(-c7ccccc7)cccc65)c4-c4ccccc4)c3)c3ccccc3-c3ccccc3)cc2)cc1]

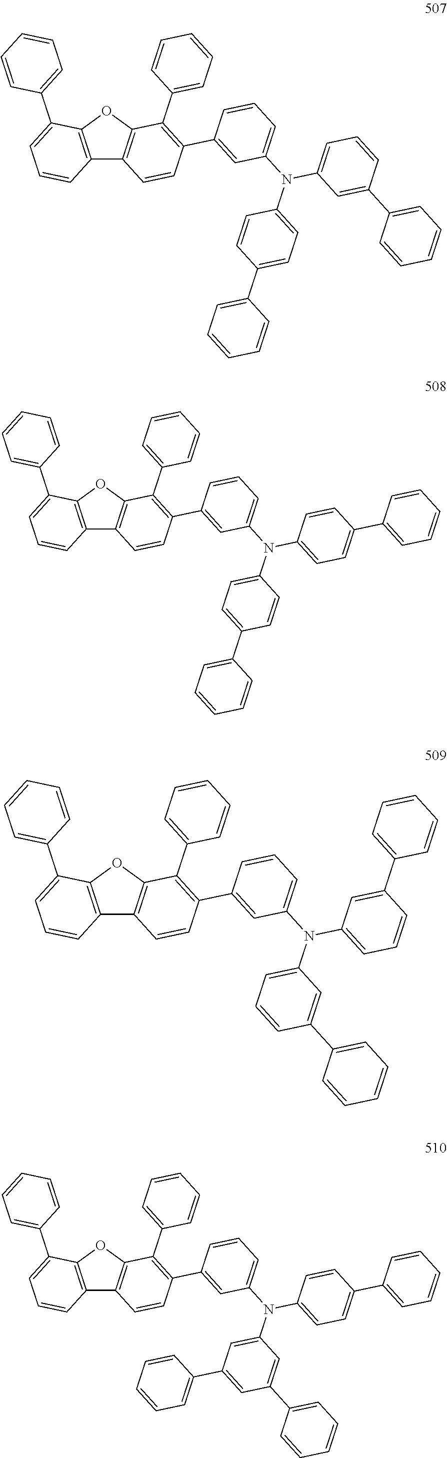 [SMILES: c1ccc(-c2ccc(N(c3cccc(-c4ccccc4)c3)c3cccc(-c4ccc5c(oc6c(-c7ccccc7)cccc65)c4-c4ccccc4)c3)cc2)cc1 ; c1ccc(-c2ccc(N(c3ccc(-c4ccccc4)cc3)c3cccc(-c4ccc5c(oc6c(-c7ccccc7)cccc65)c4-c4ccccc4)c3)cc2)cc1 ; c1ccc(-c2cccc(N(c3cccc(-c4ccccc4)c3)c3cccc(-c4ccc5c(oc6c(-c7ccccc7)cccc65)c4-c4ccccc4)c3)c2)cc1 ; c1ccc(-c2ccc(N(c3cc(-c4ccccc4)cc(-c4ccccc4)c3)c3cccc(-c4ccc5c(oc6c(-c7ccccc7)cccc65)c4-c4ccccc4)c3)cc2)cc1]

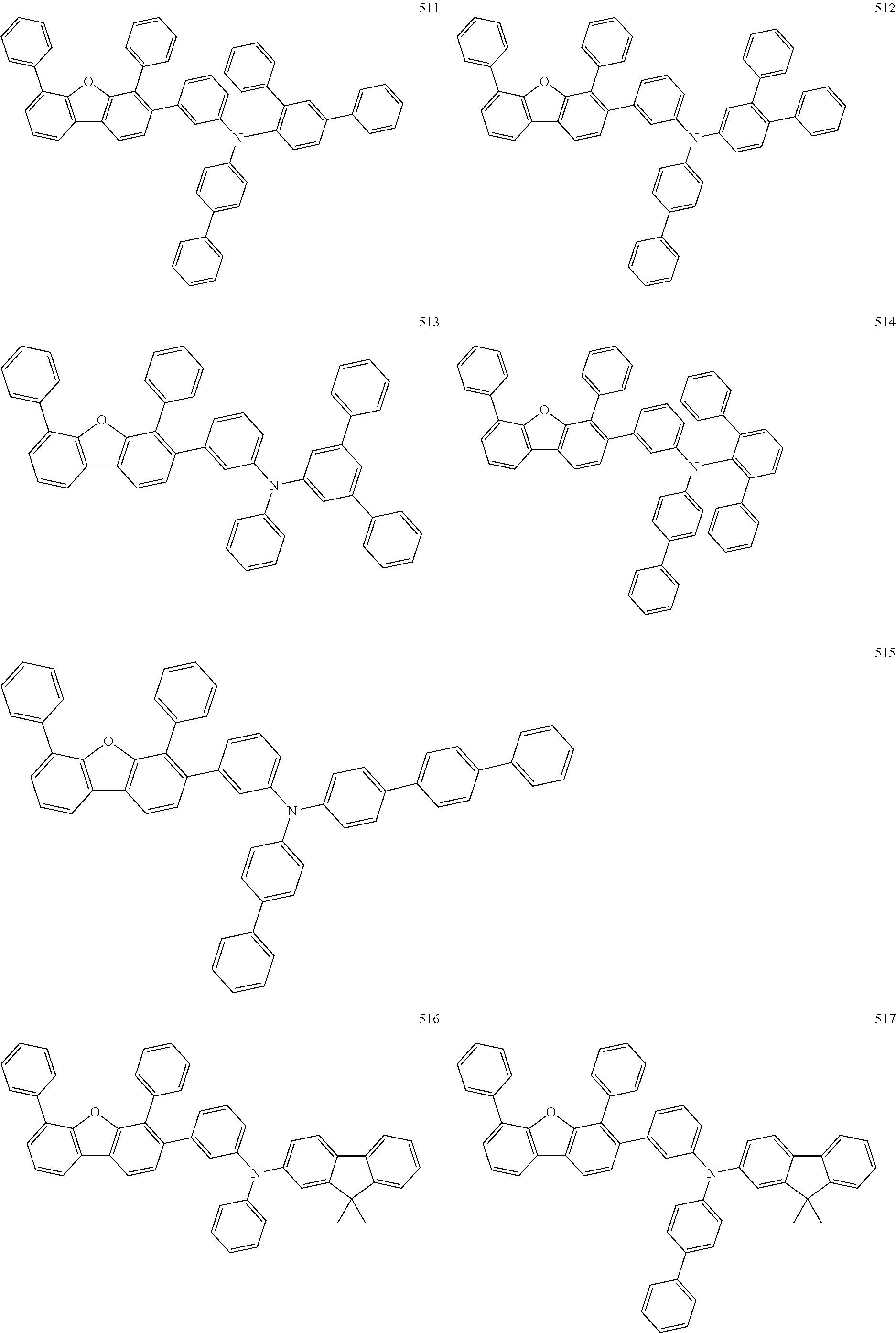

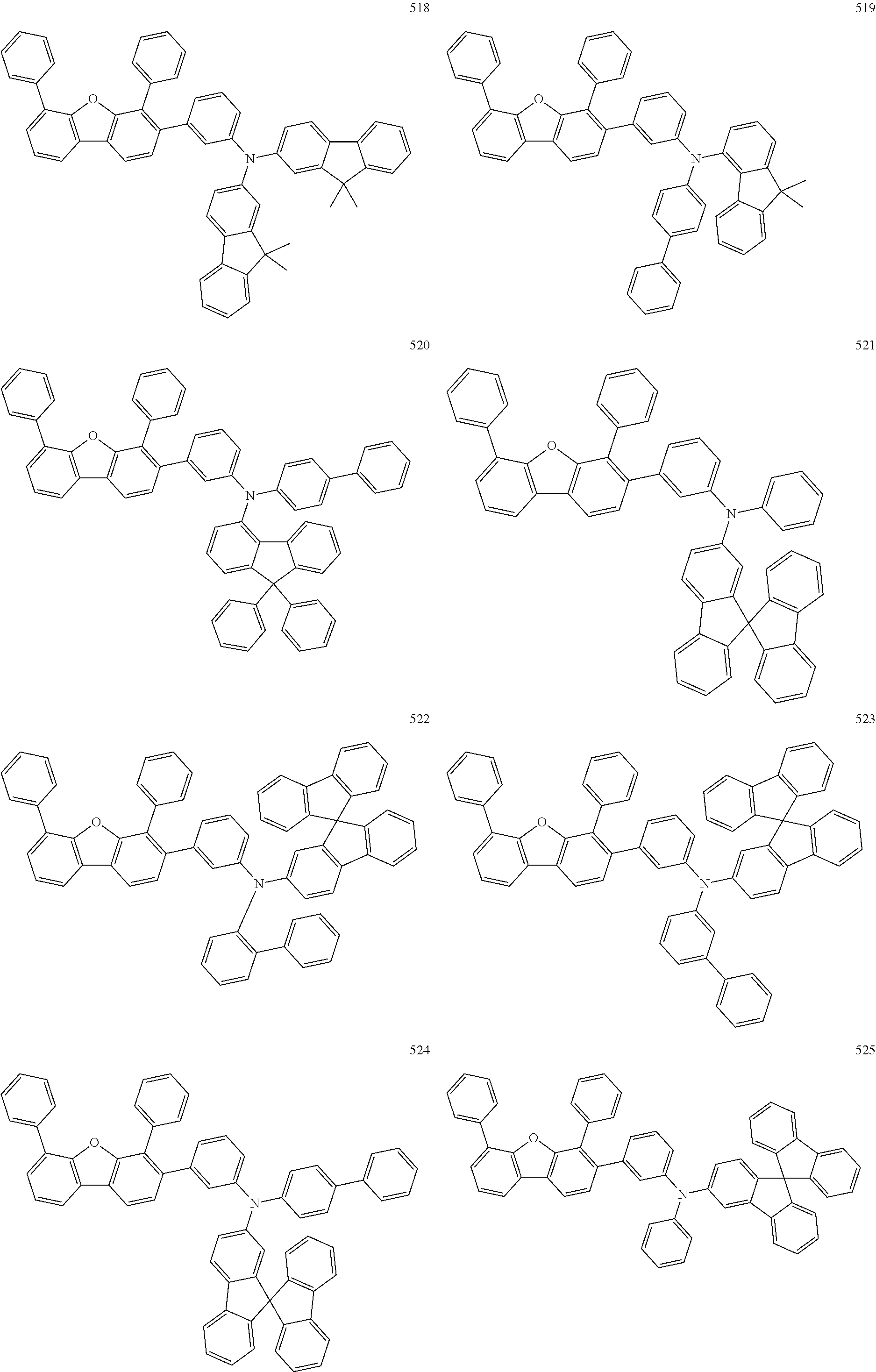

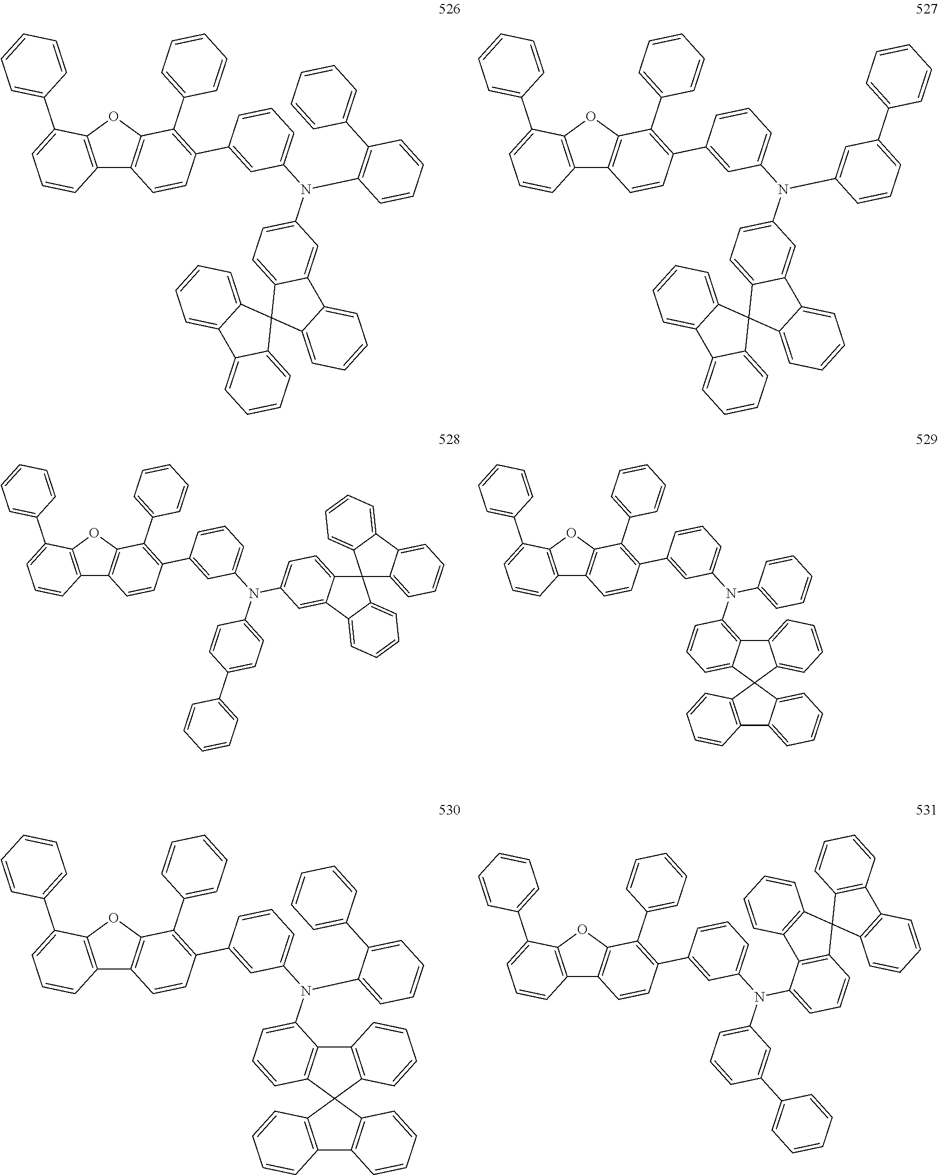

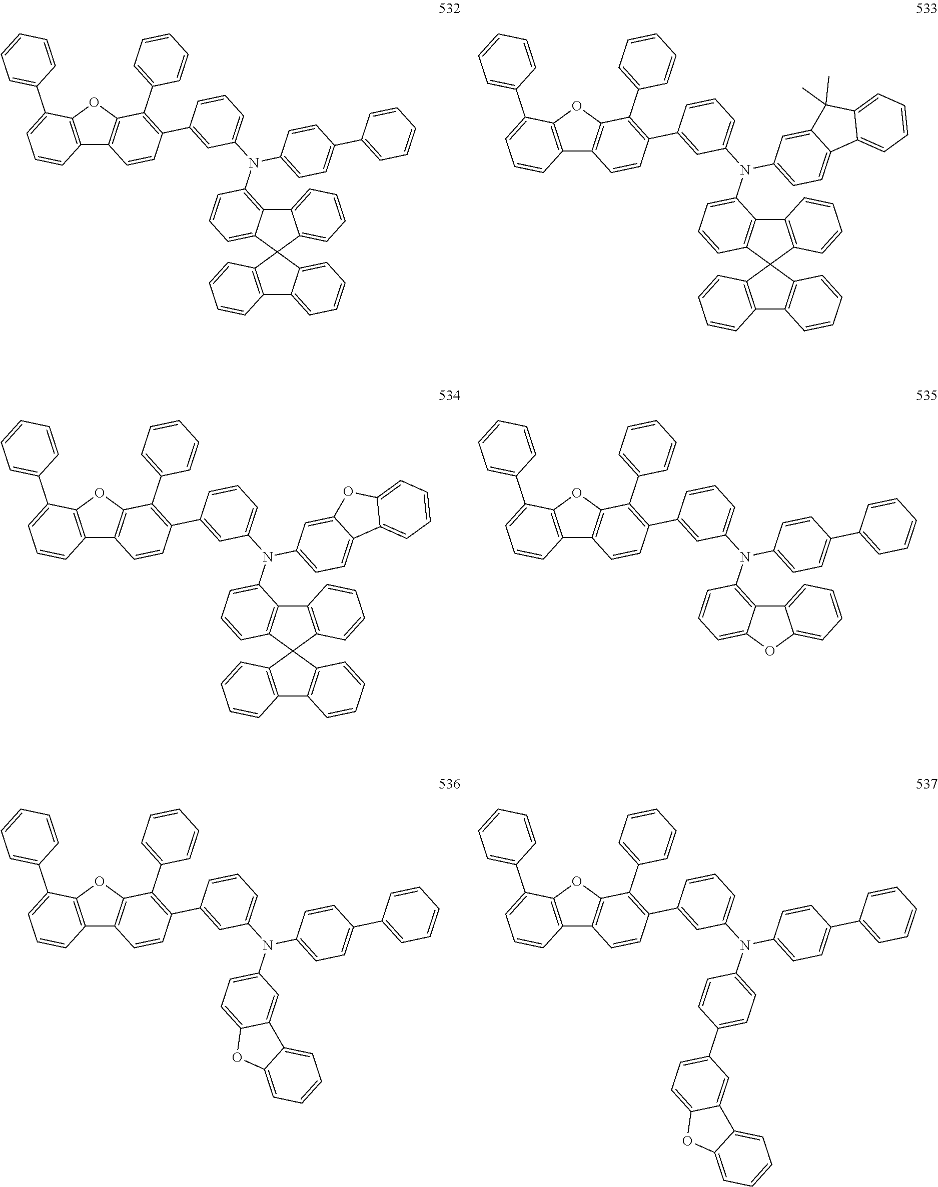

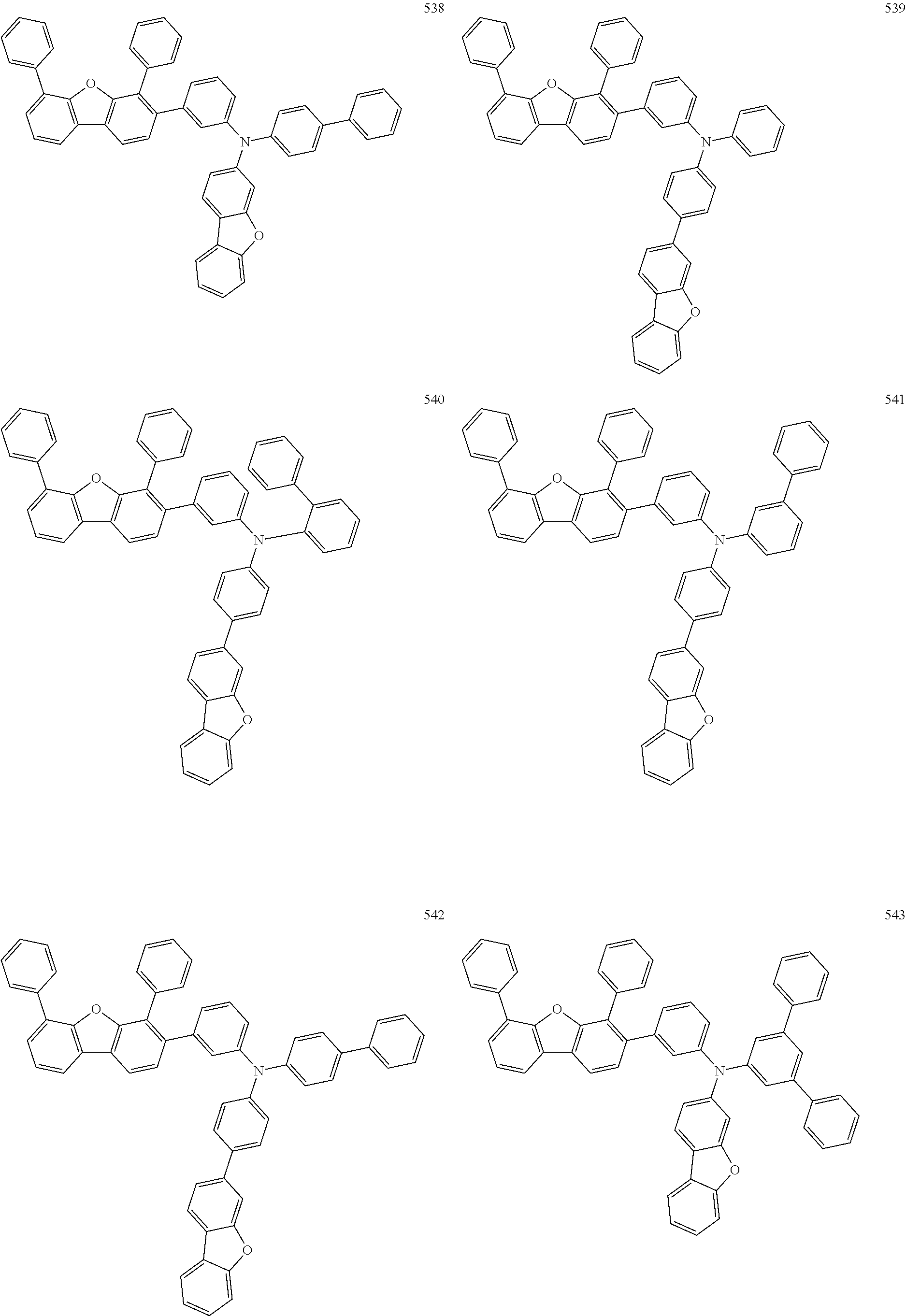

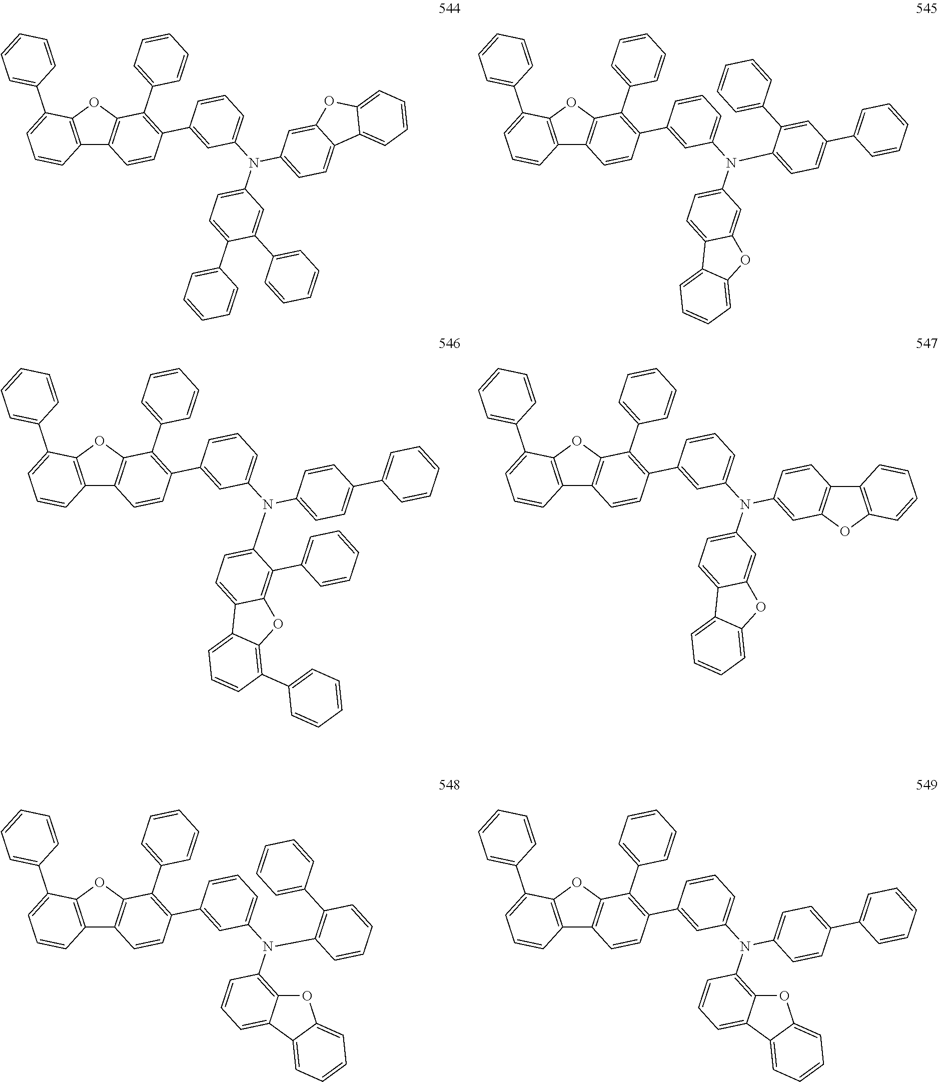

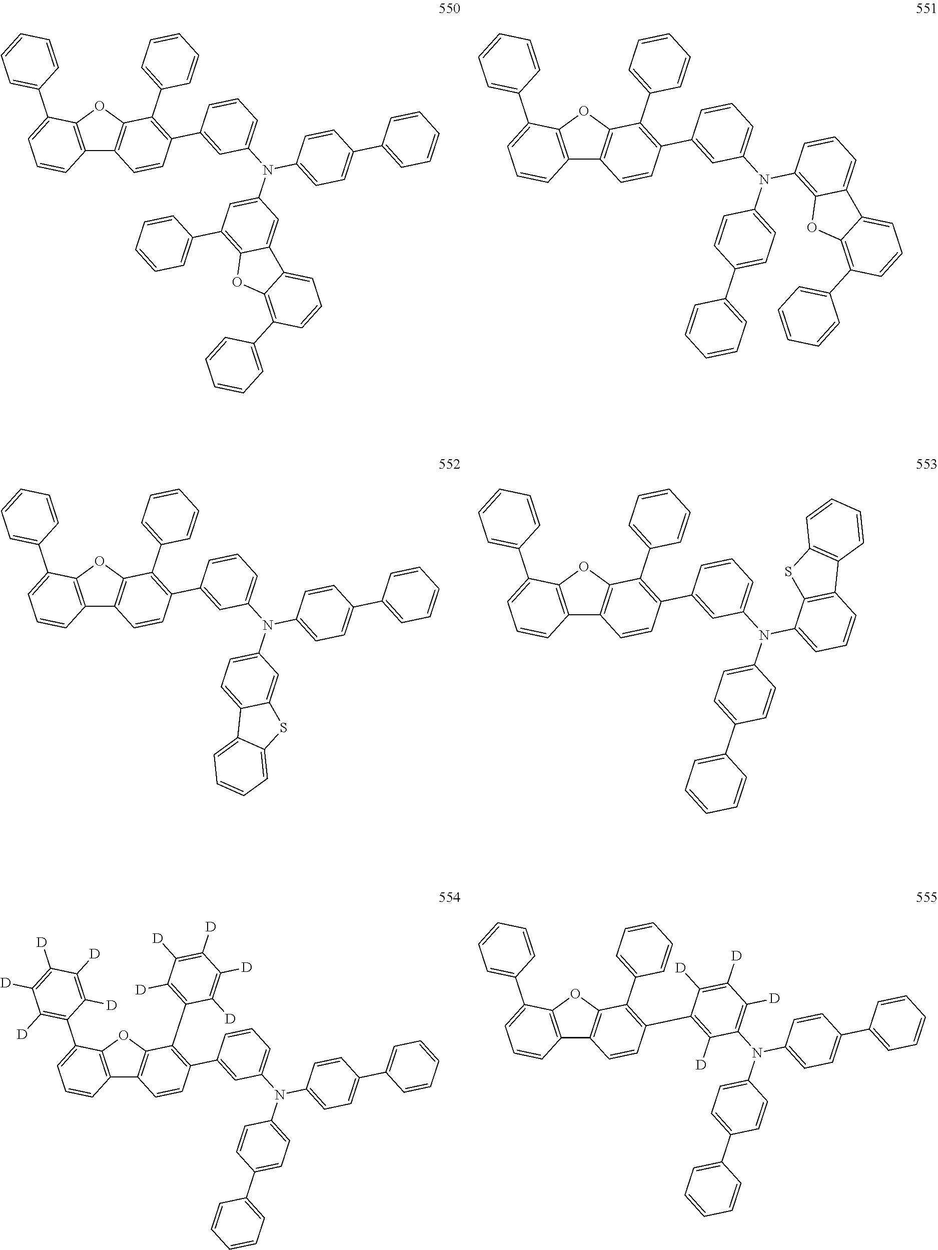

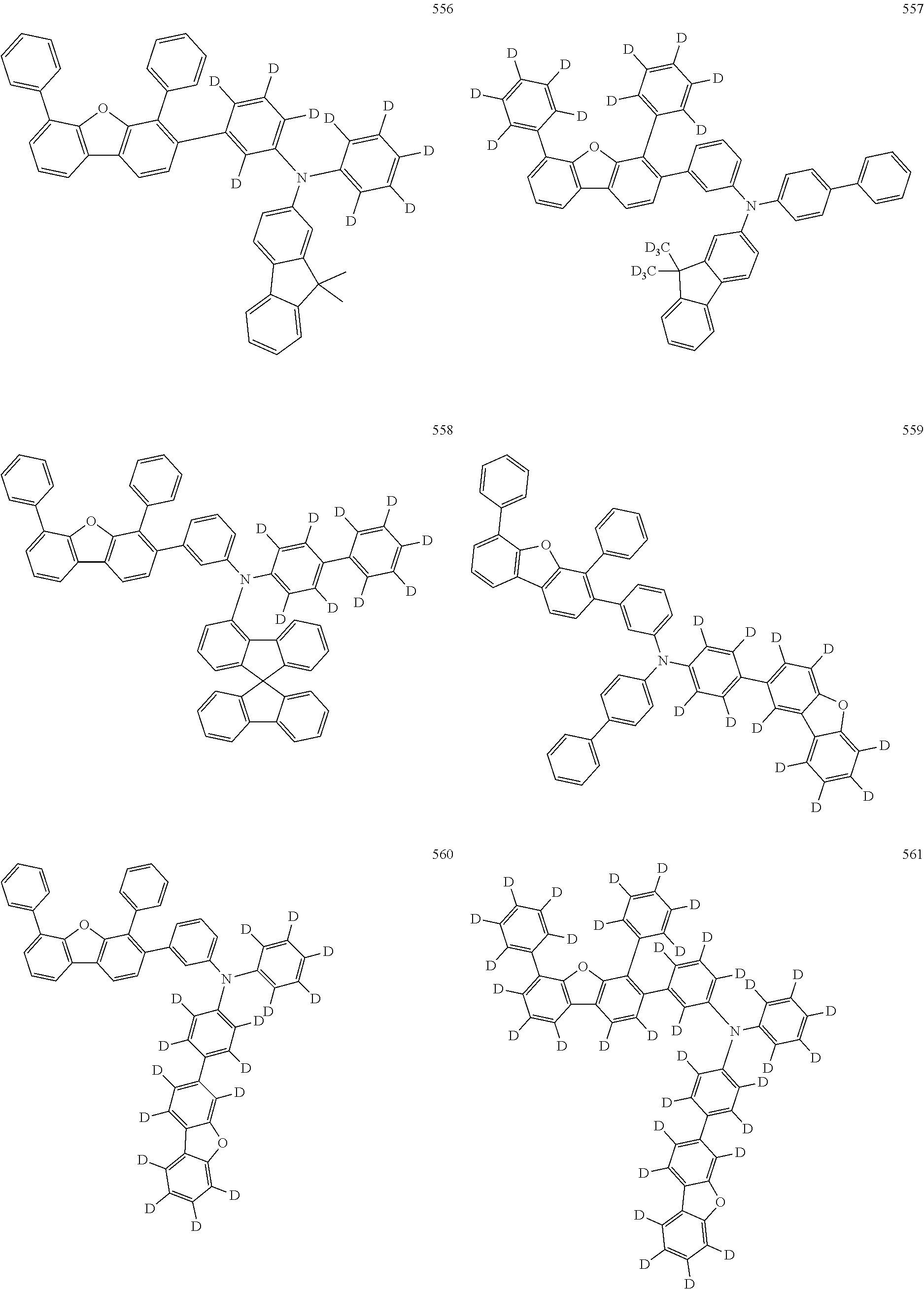

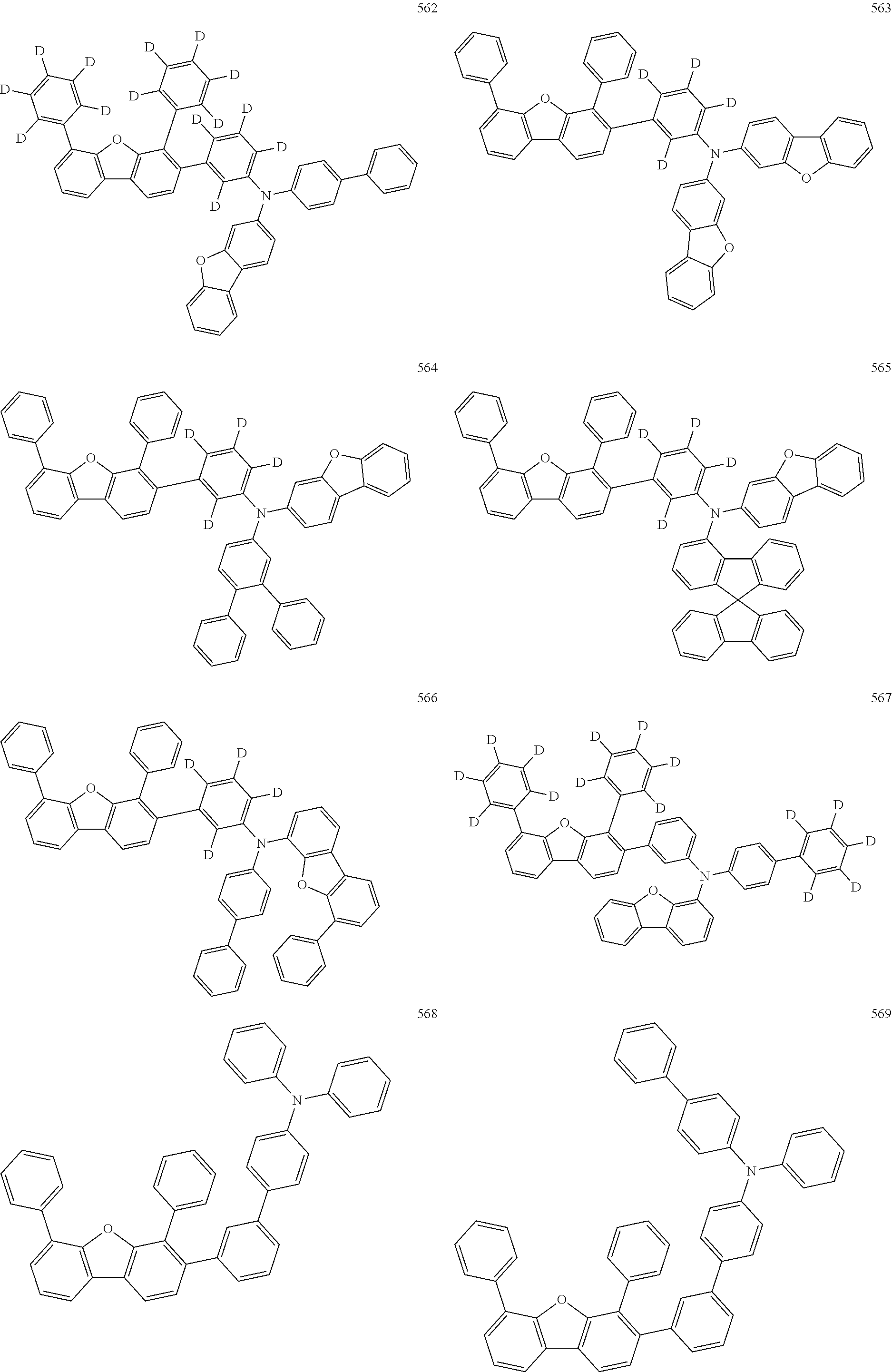

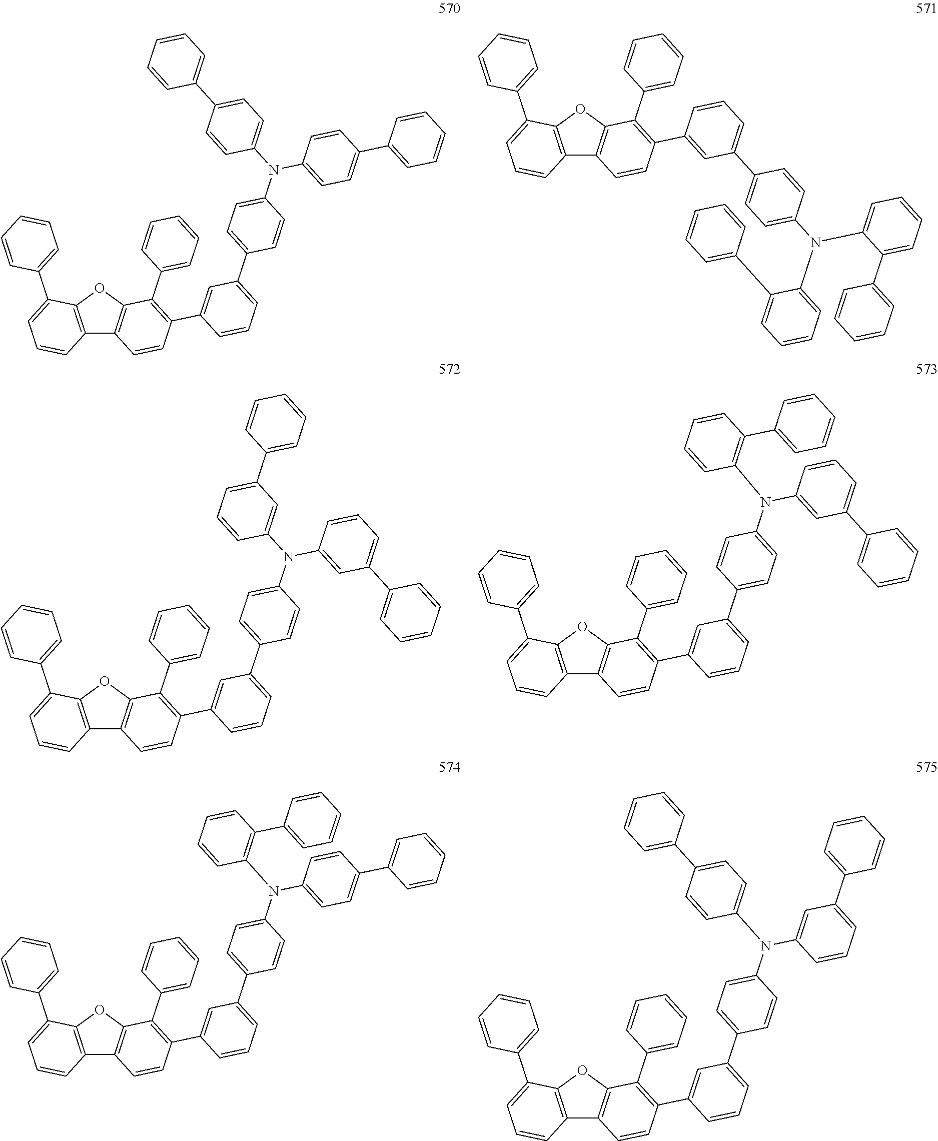

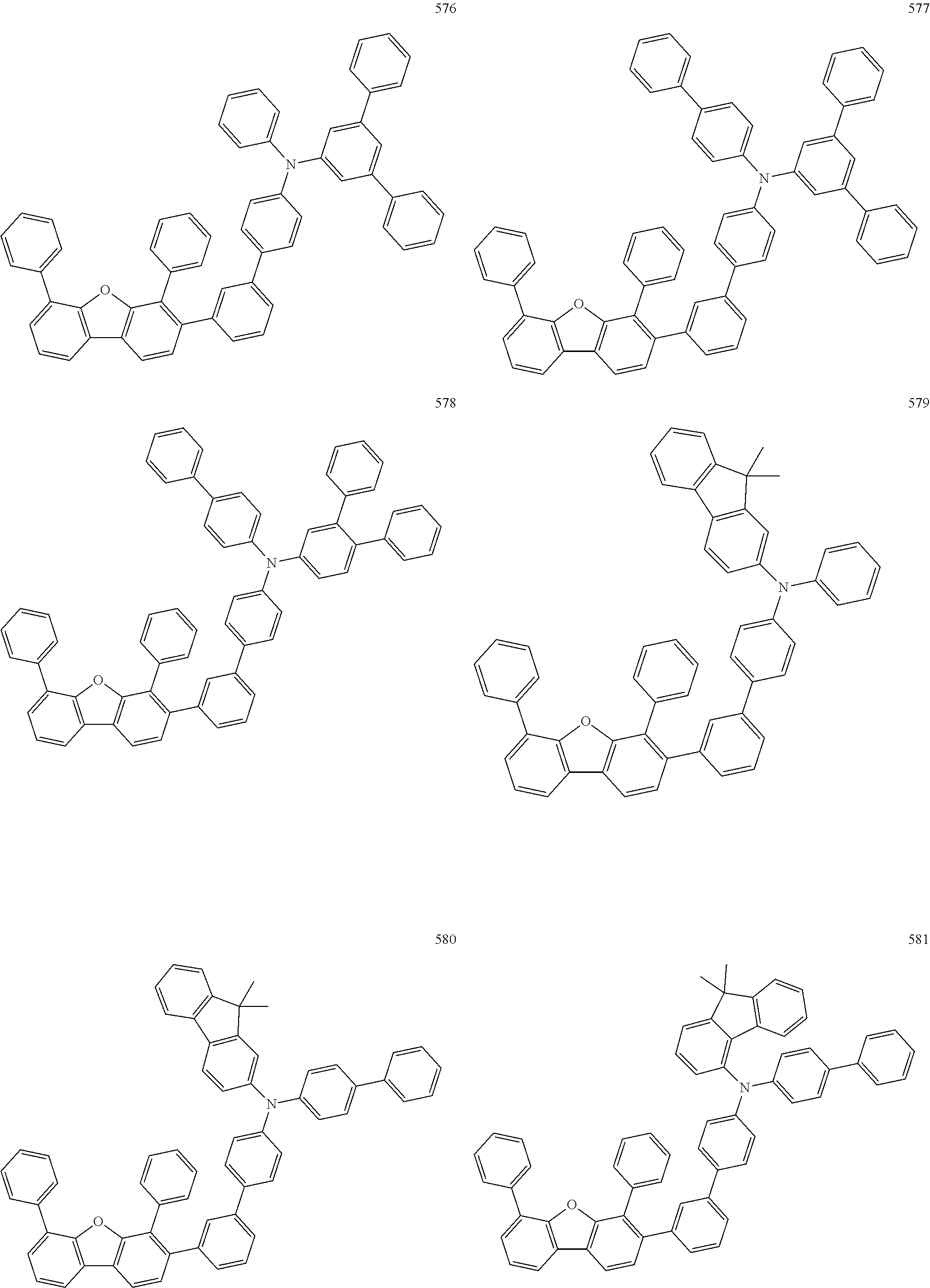

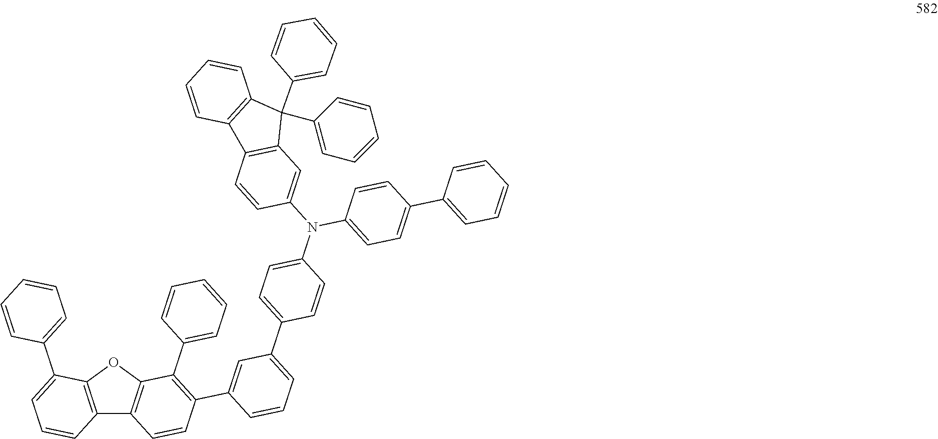 [SMILES: c1ccc(-c2ccc(N(c3ccc(-c4cccc(-c5ccc6c(oc7c(-c8ccccc8)cccc76)c5-c5ccccc5)c4)cc3)c3ccc4c(c3)C(c3ccccc3)(c3ccccc3)c3ccccc3-4)cc2)cc1]

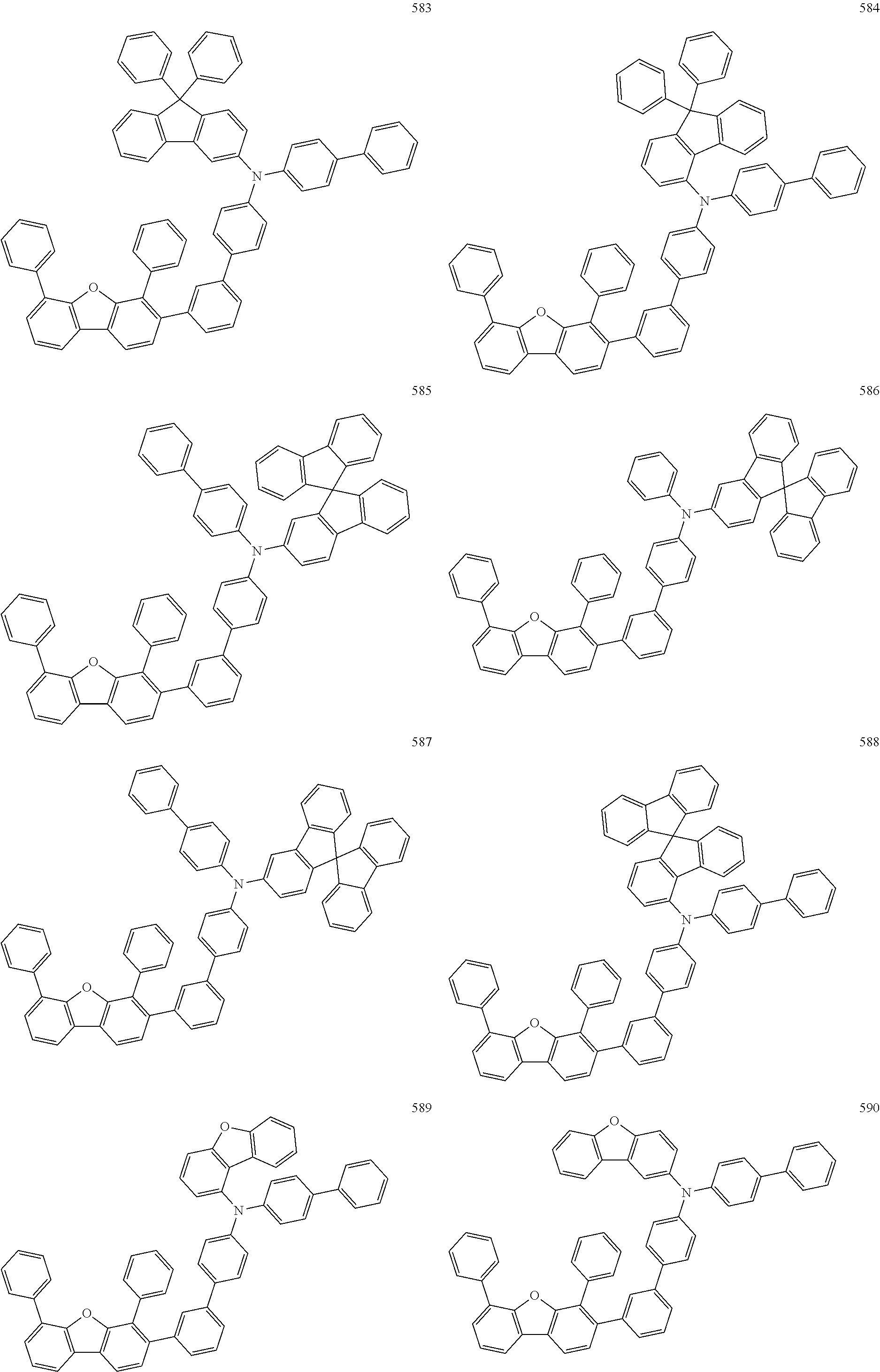

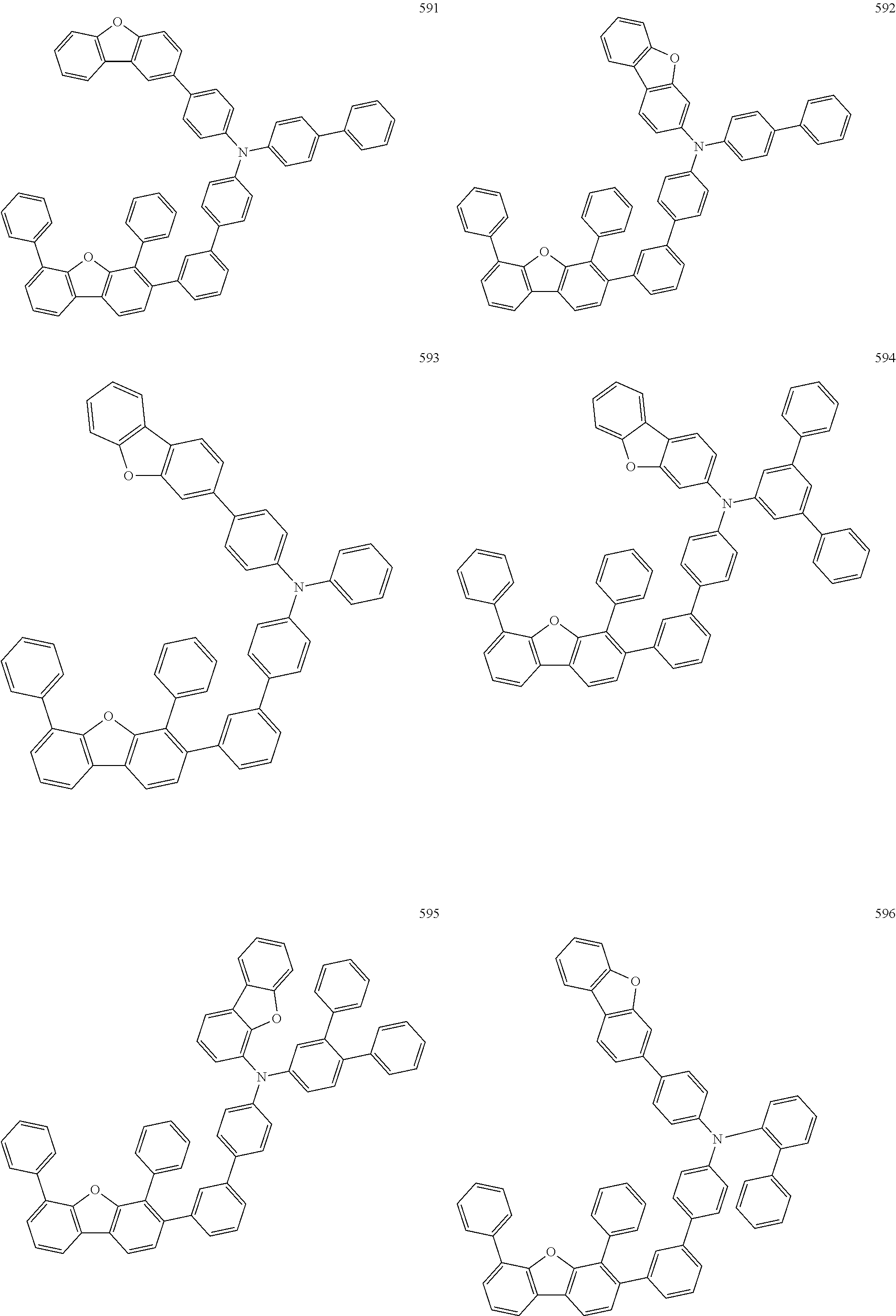

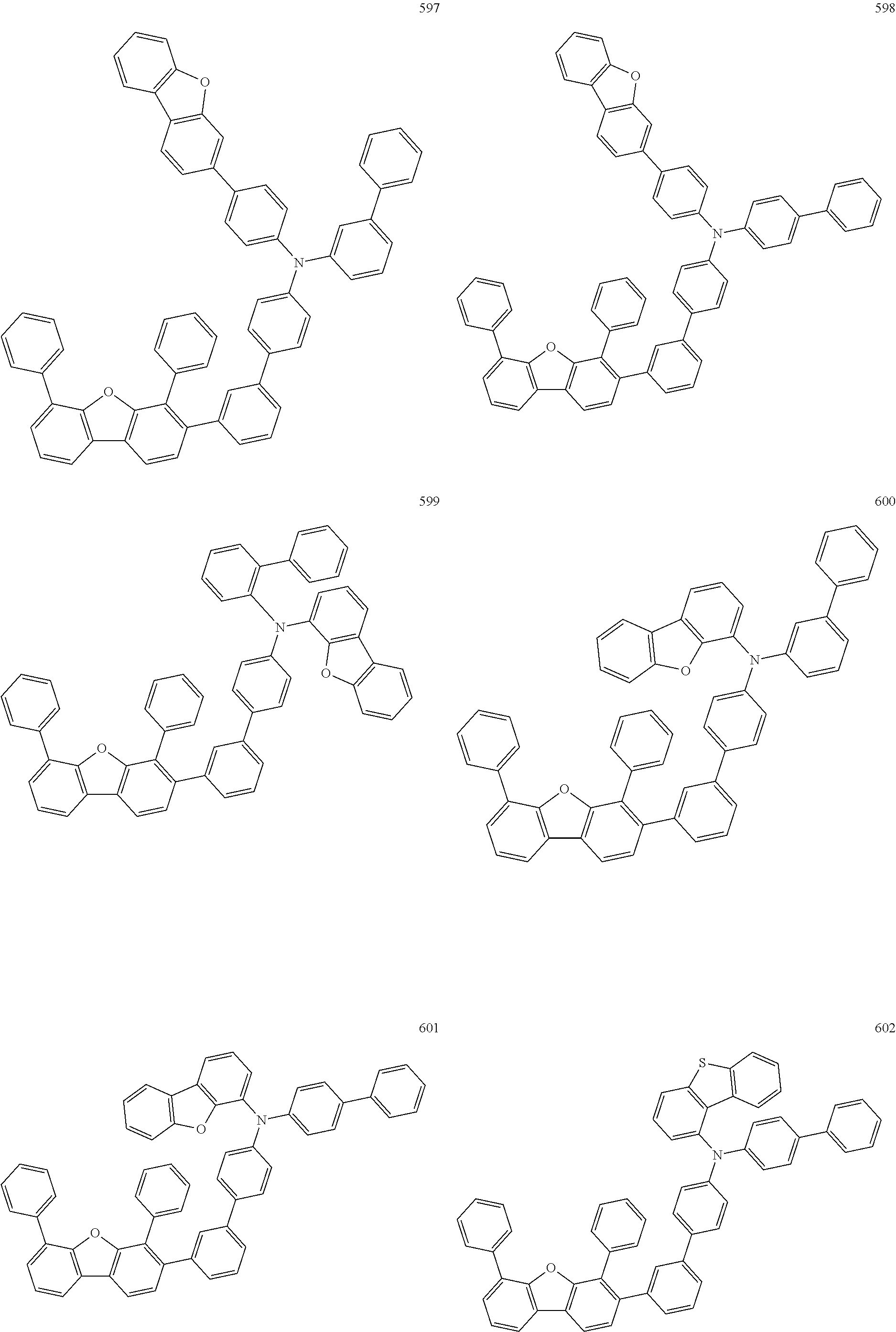

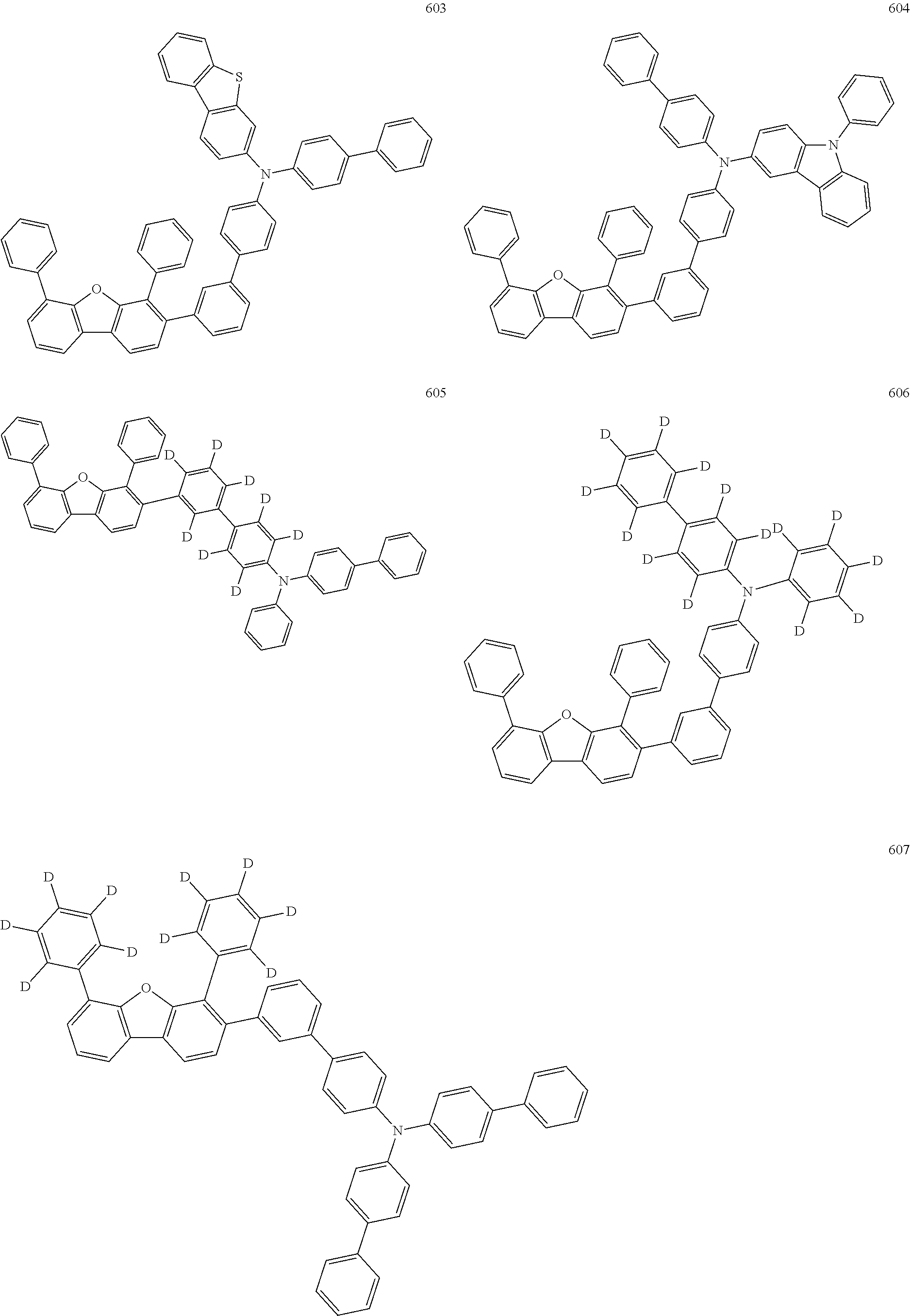

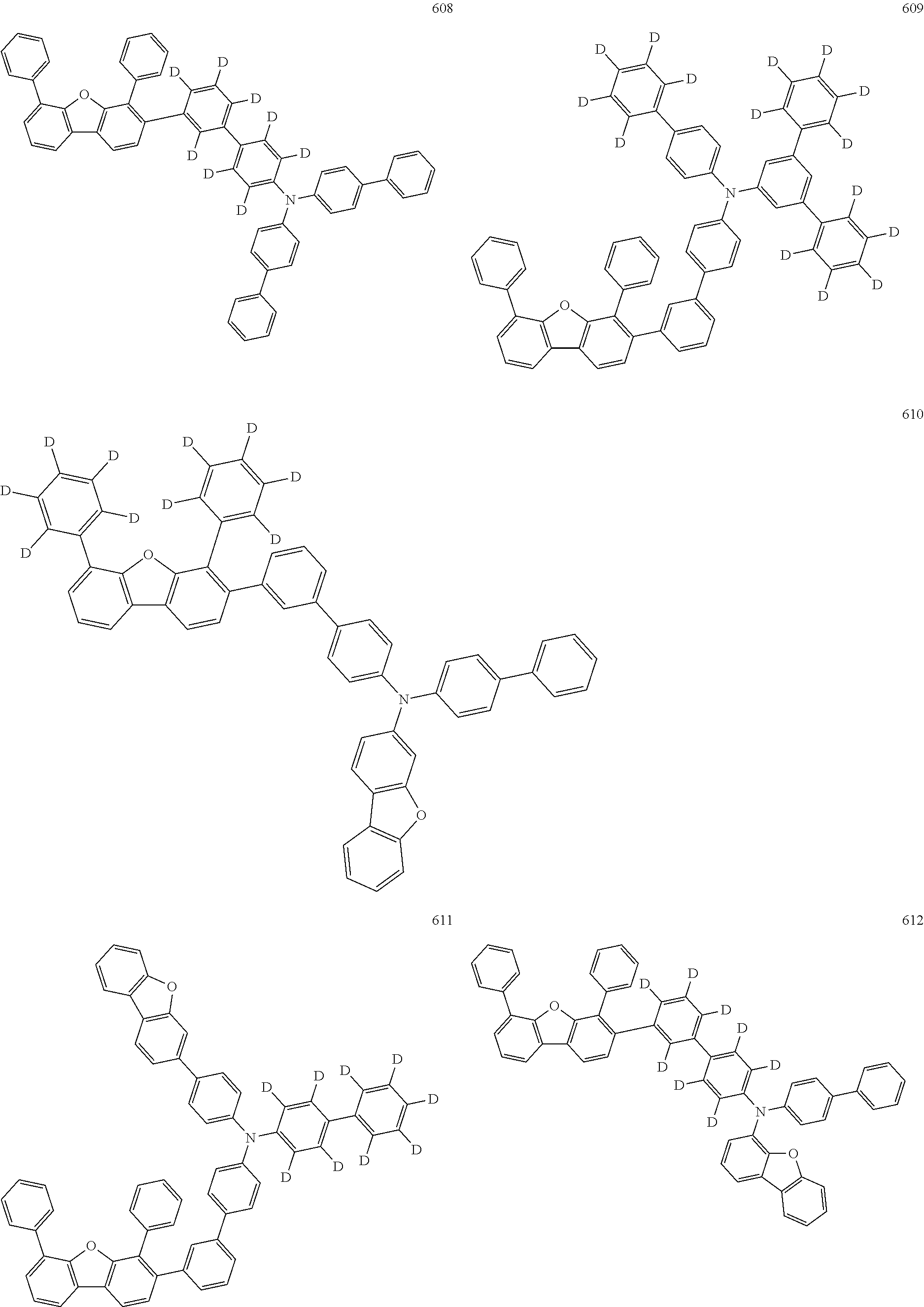

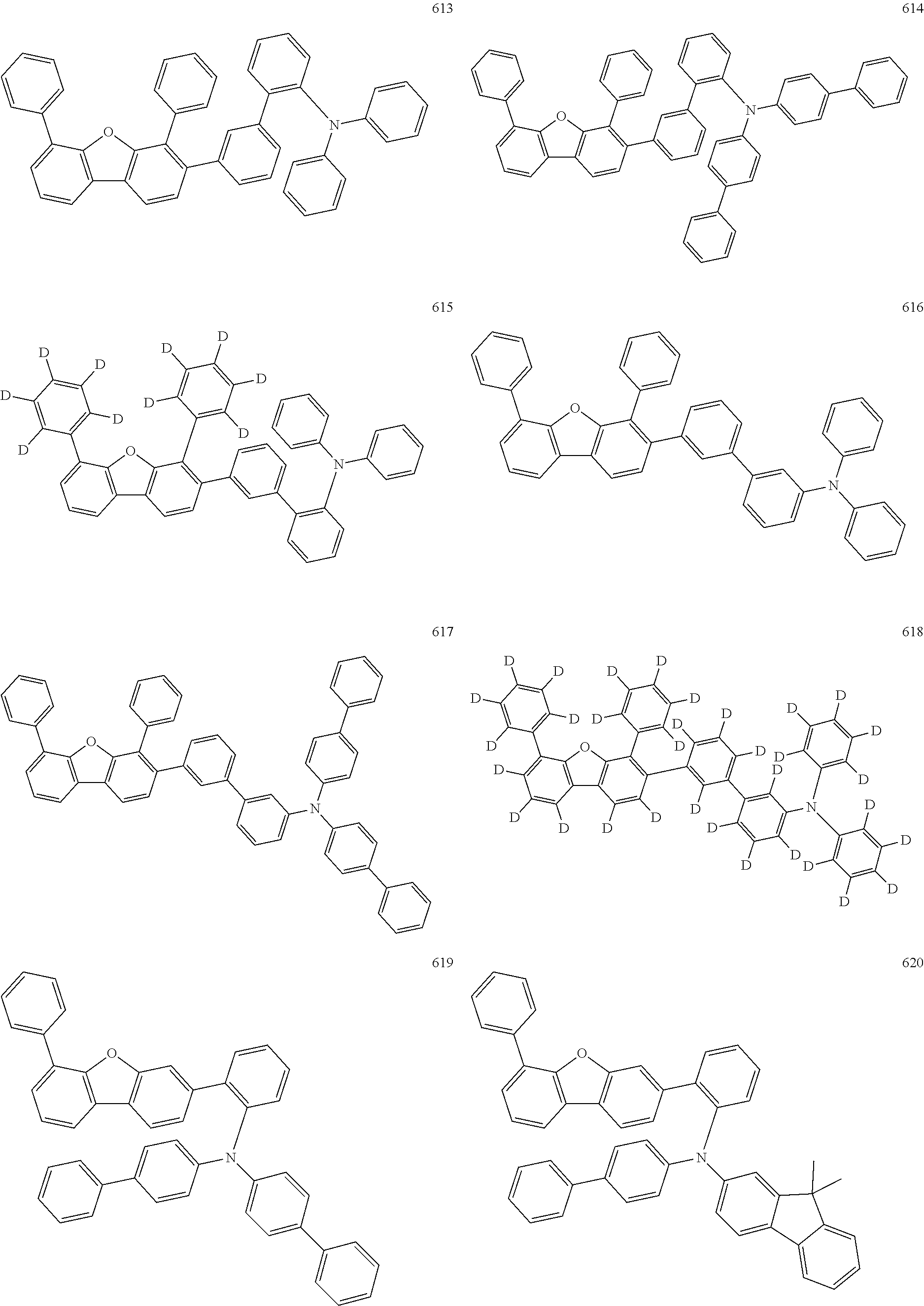

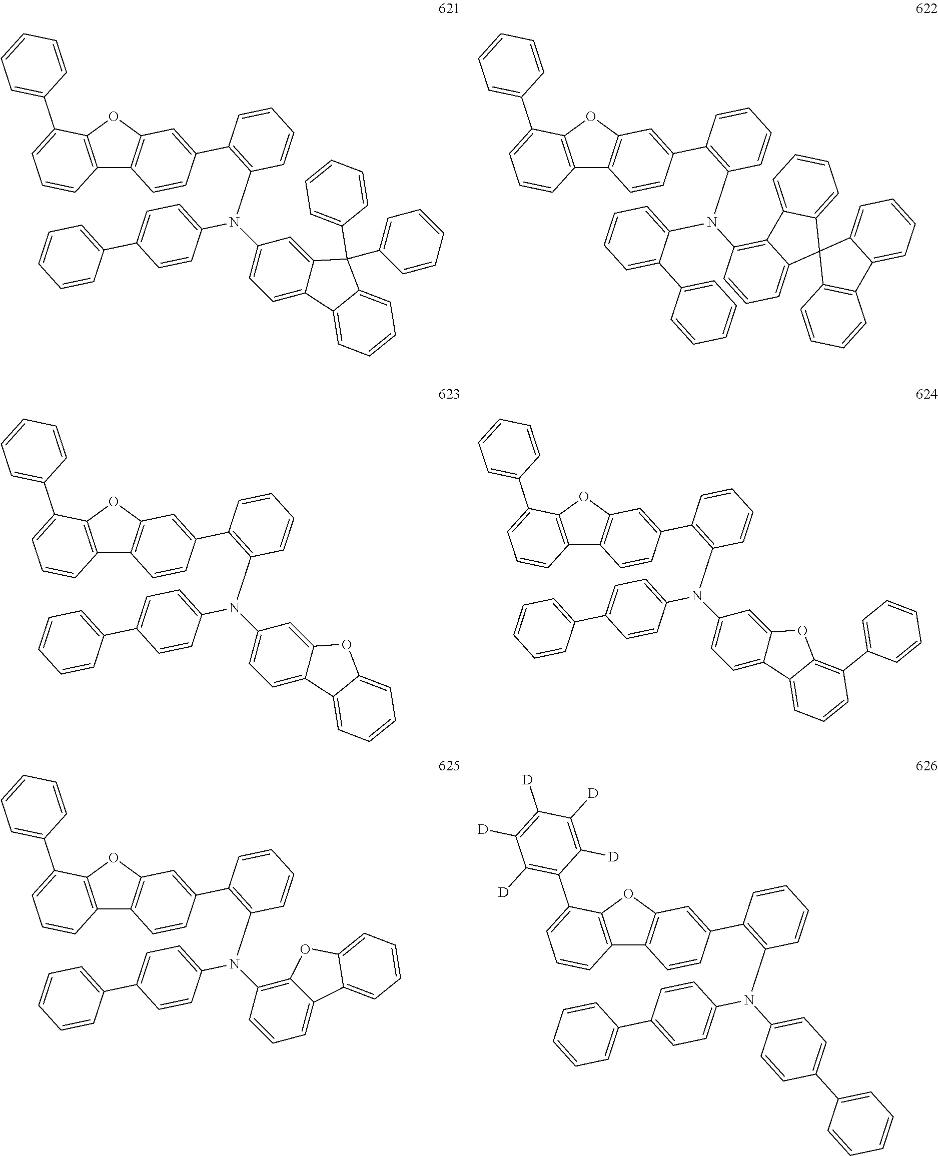

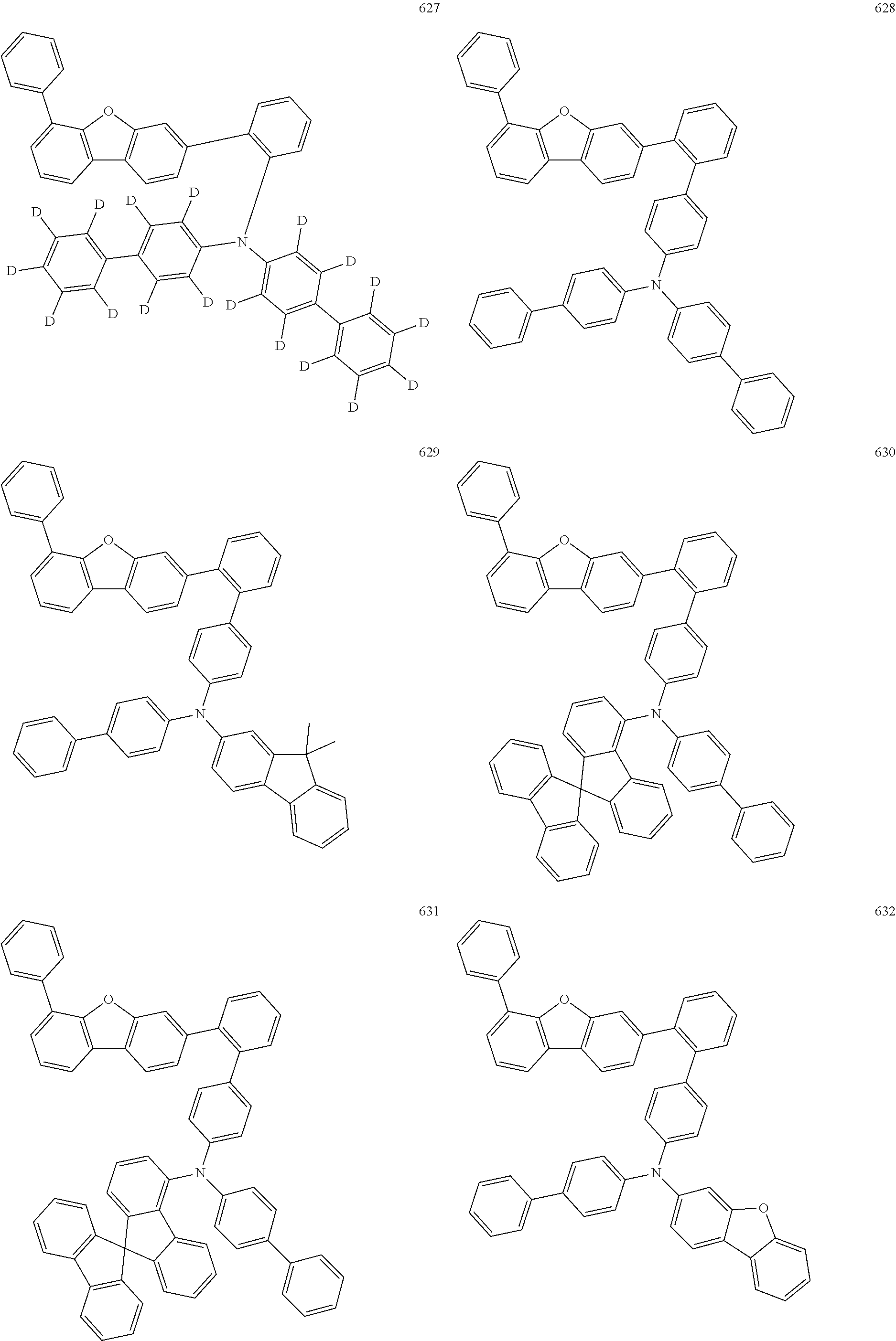

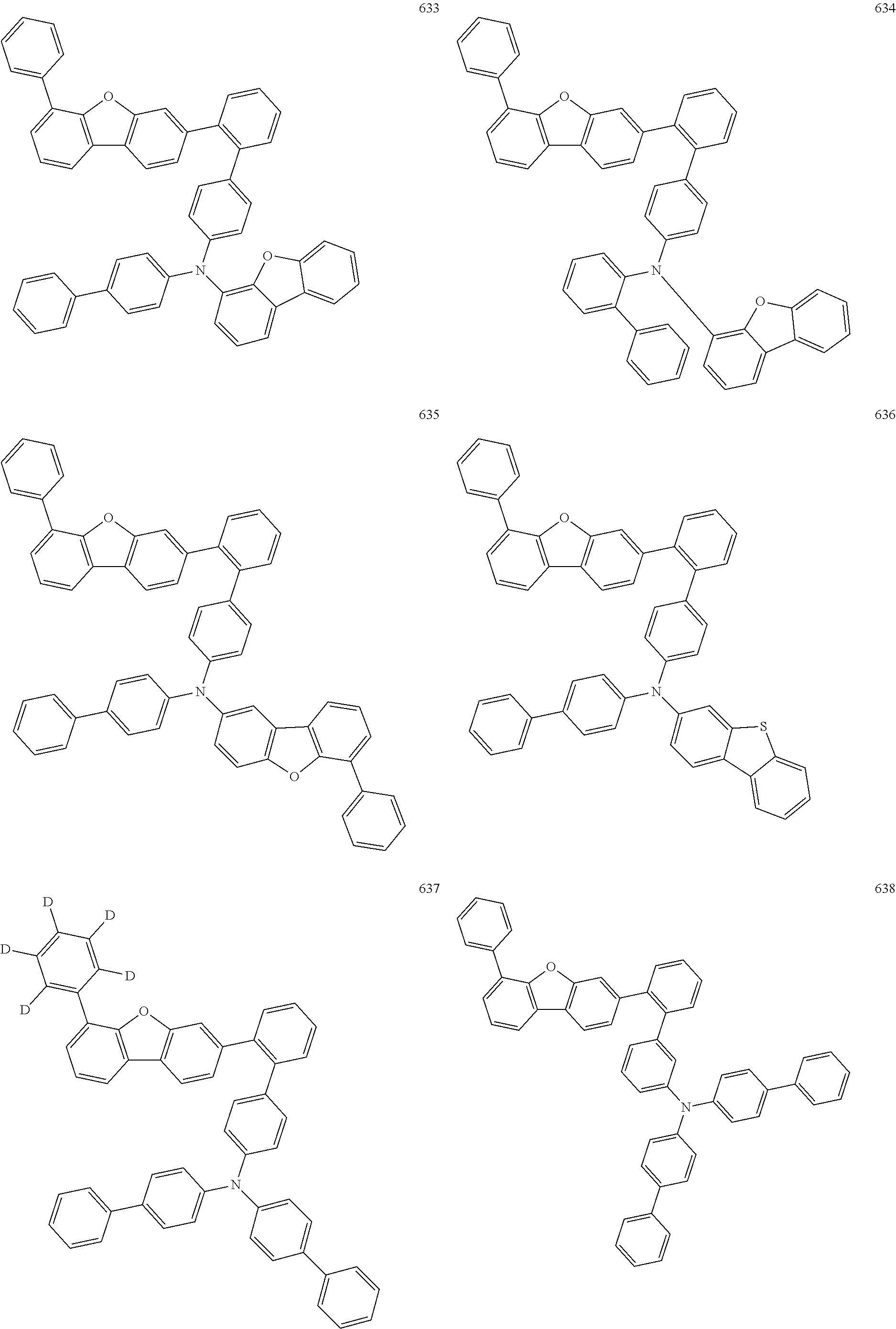

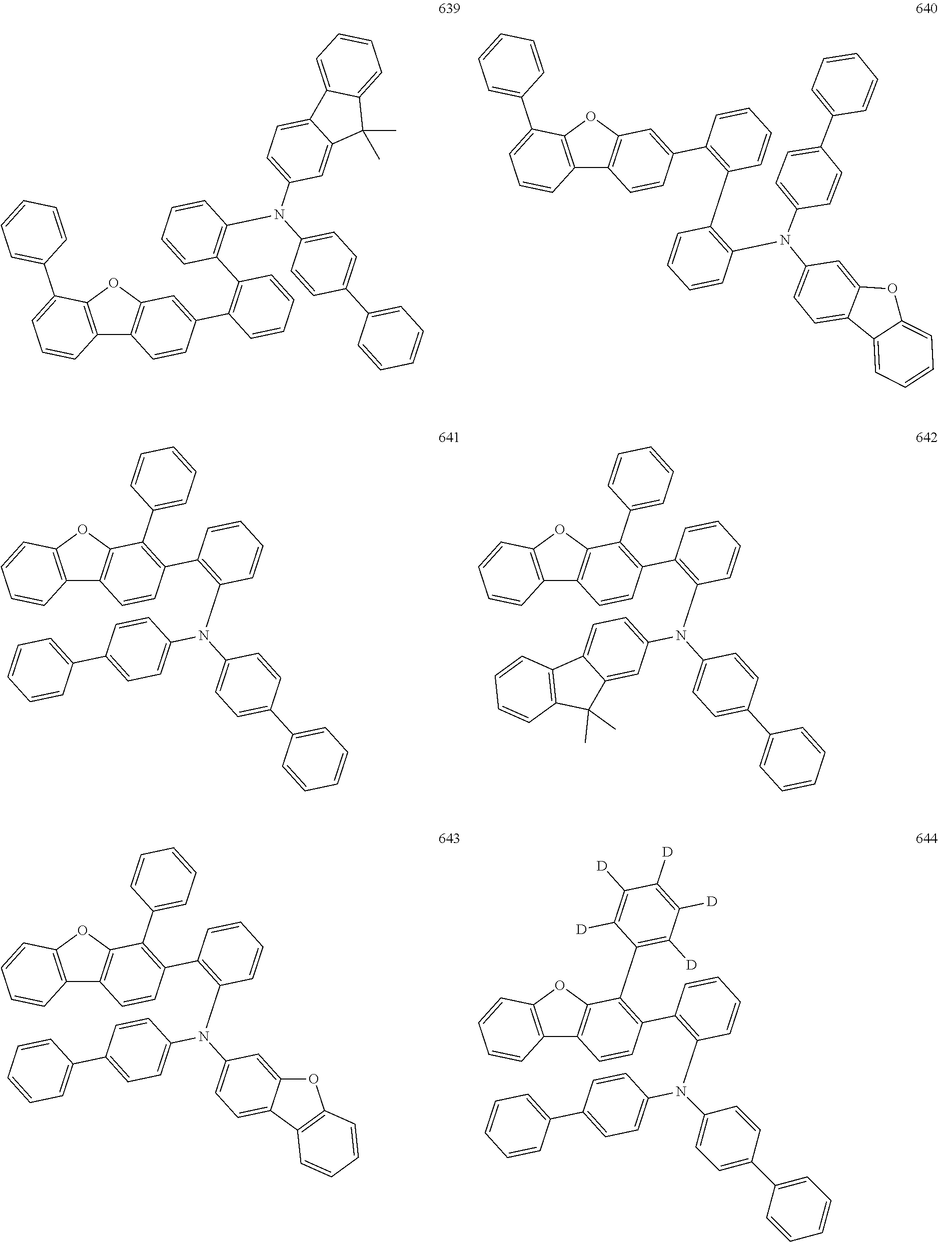

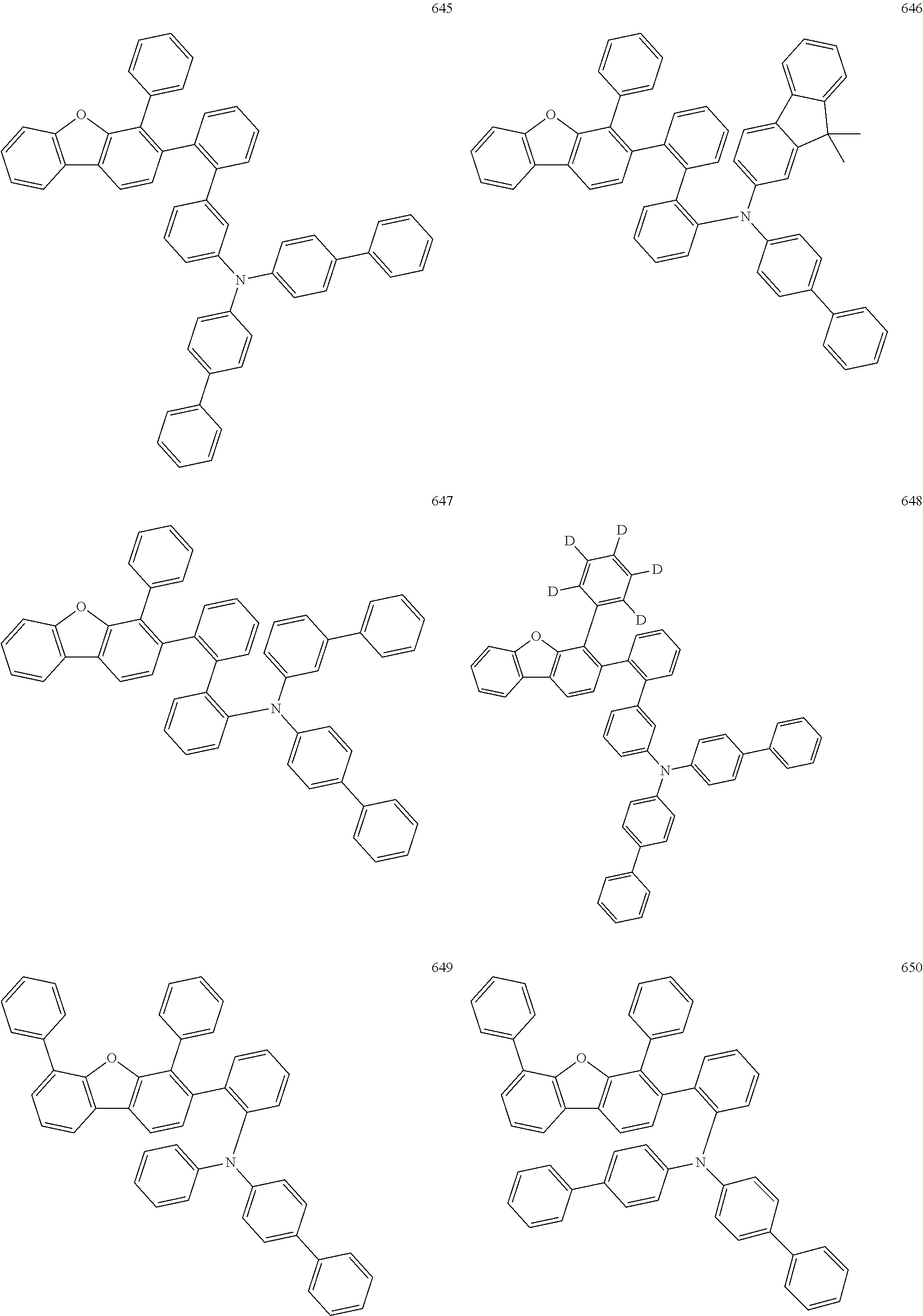

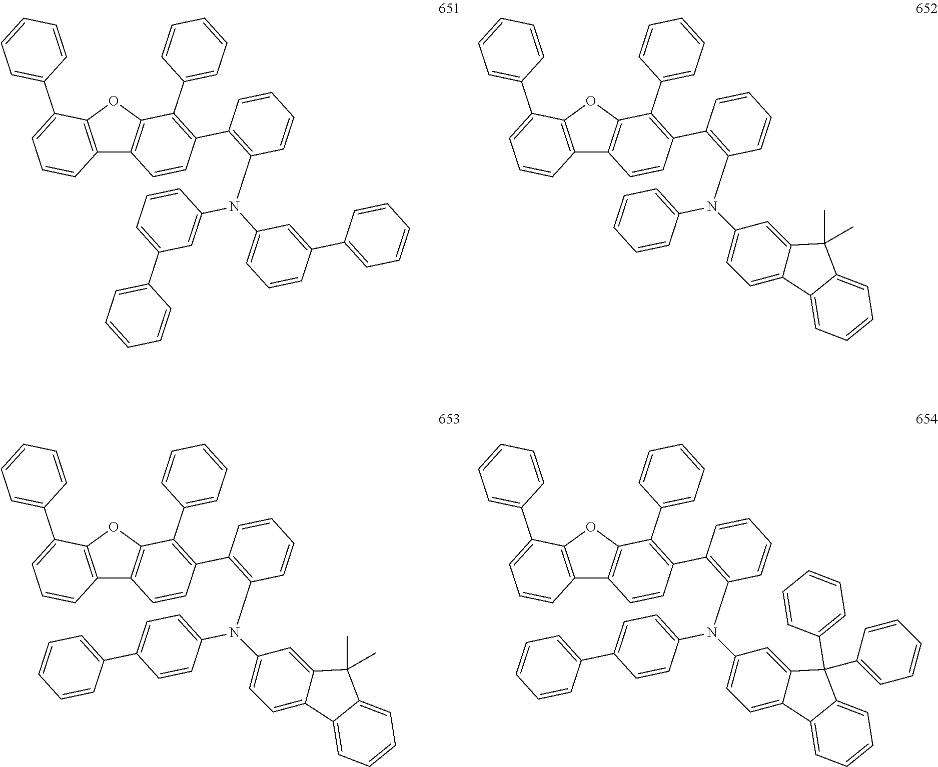 [SMILES: c1ccc(-c2cccc(N(c3cccc(-c4ccccc4)c3)c3ccccc3-c3ccc4c(oc5c(-c6ccccc6)cccc54)c3-c3ccccc3)c2)cc1 ; CC1(C)c2ccccc2-c2ccc(N(c3ccccc3)c3ccccc3-c3ccc4c(oc5c(-c6ccccc6)cccc54)c3-c3ccccc3)cc21 ; CC1(C)c2ccccc2-c2ccc(N(c3ccc(-c4ccccc4)cc3)c3ccccc3-c3ccc4c(oc5c(-c6ccccc6)cccc54)c3-c3ccccc3)cc21 ; c1ccc(-c2ccc(N(c3ccc4c(c3)C(c3ccccc3)(c3ccccc3)c3ccccc3-4)c3ccccc3-c3ccc4c(oc5c(-c6ccccc6)cccc54)c3-c3ccccc3)cc2)cc1]

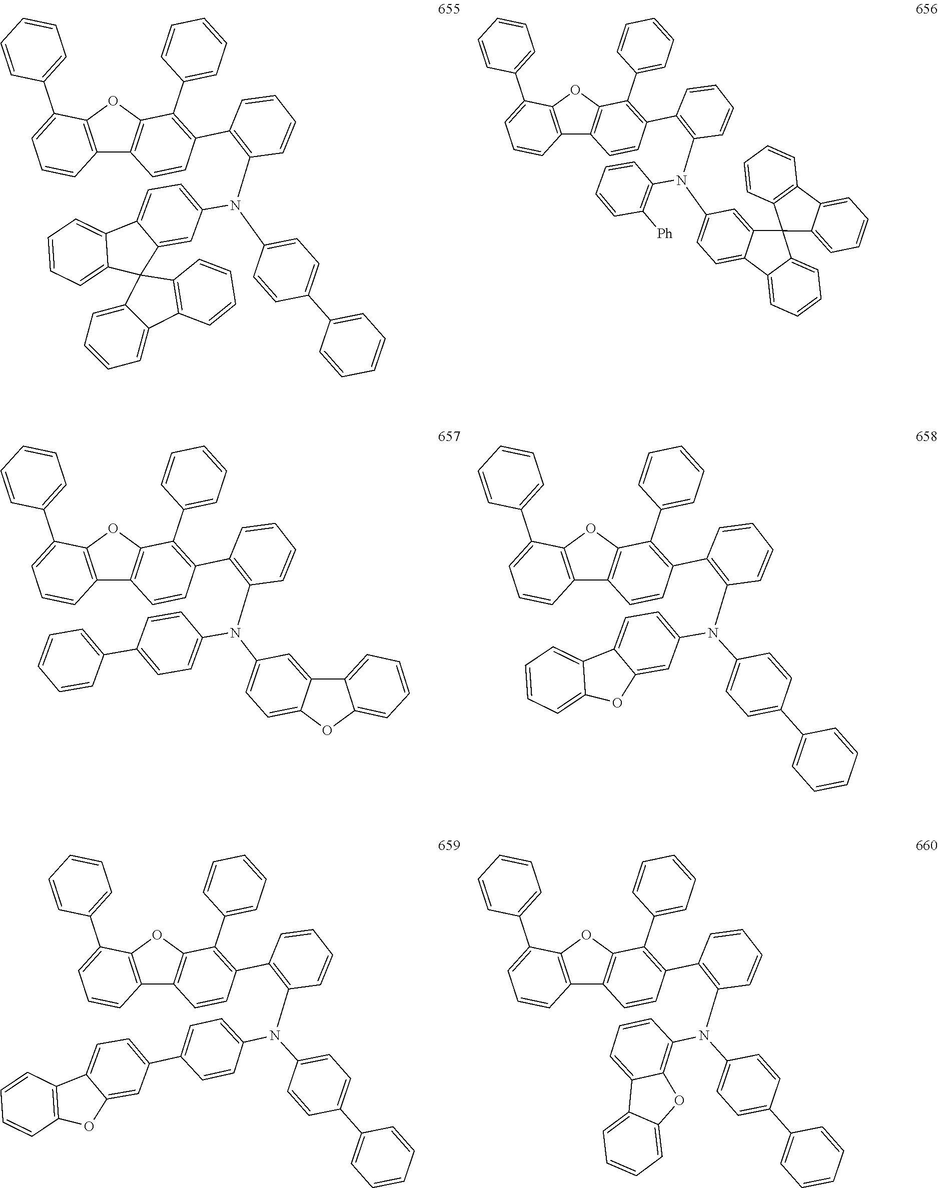

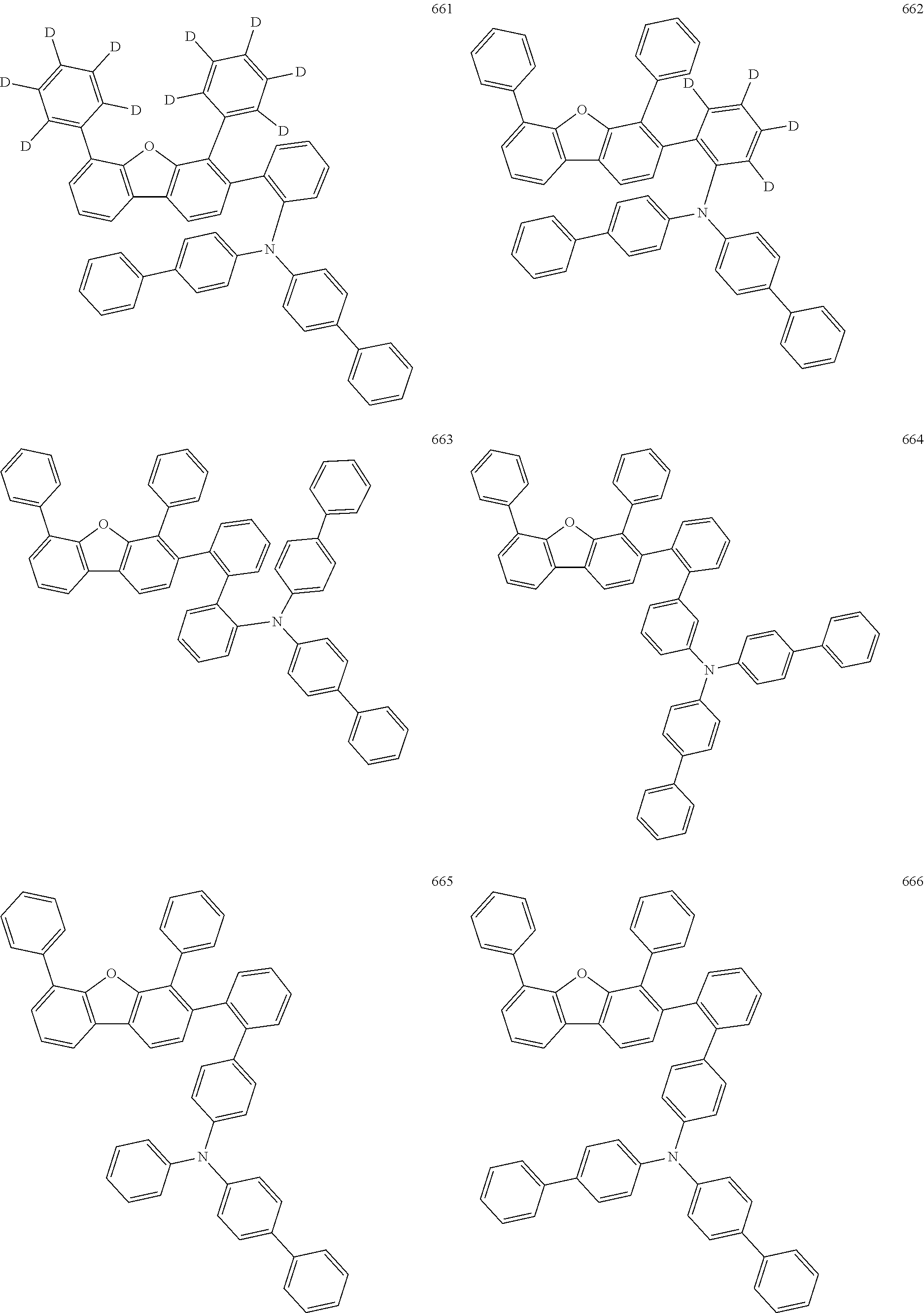

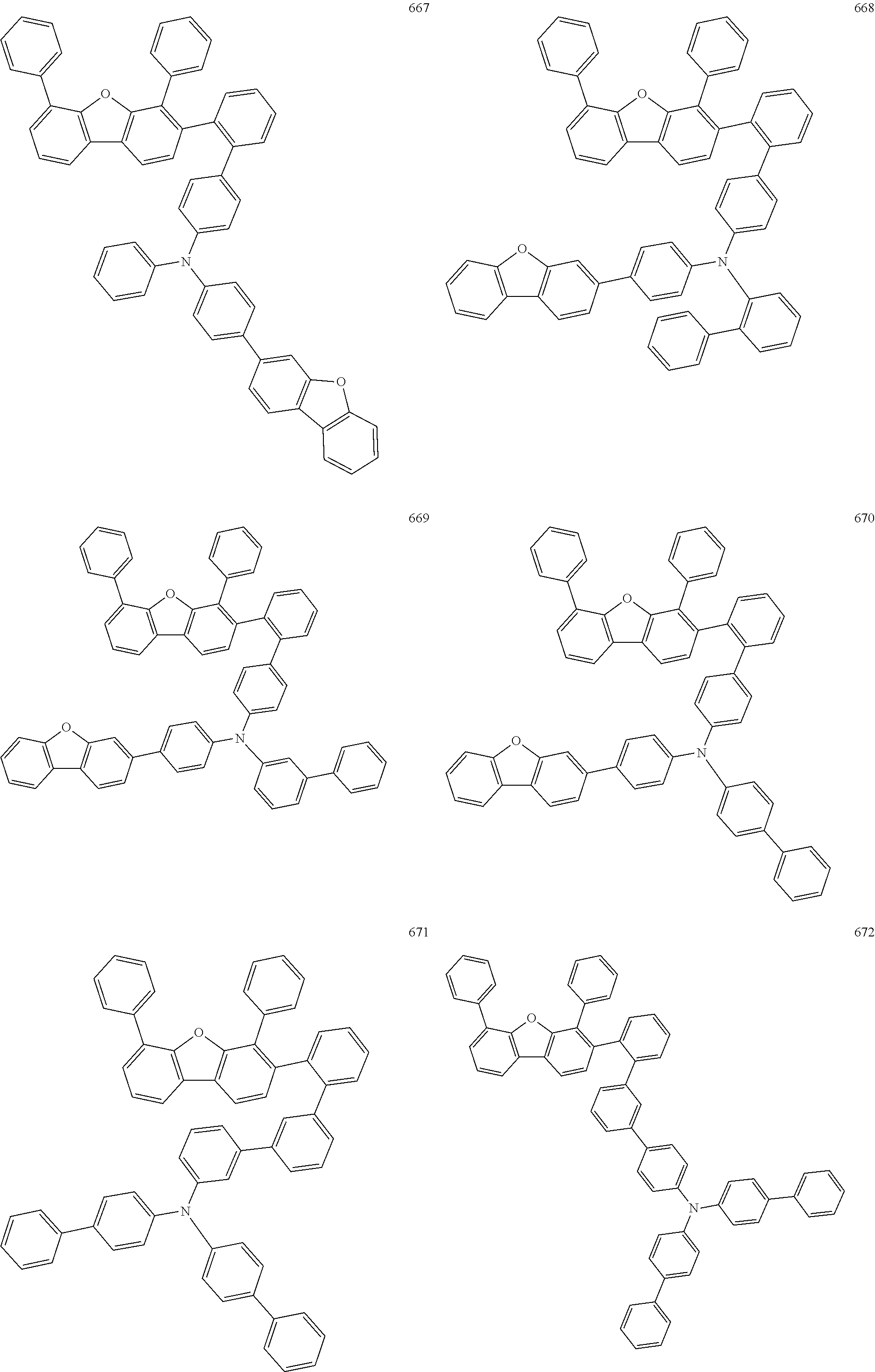

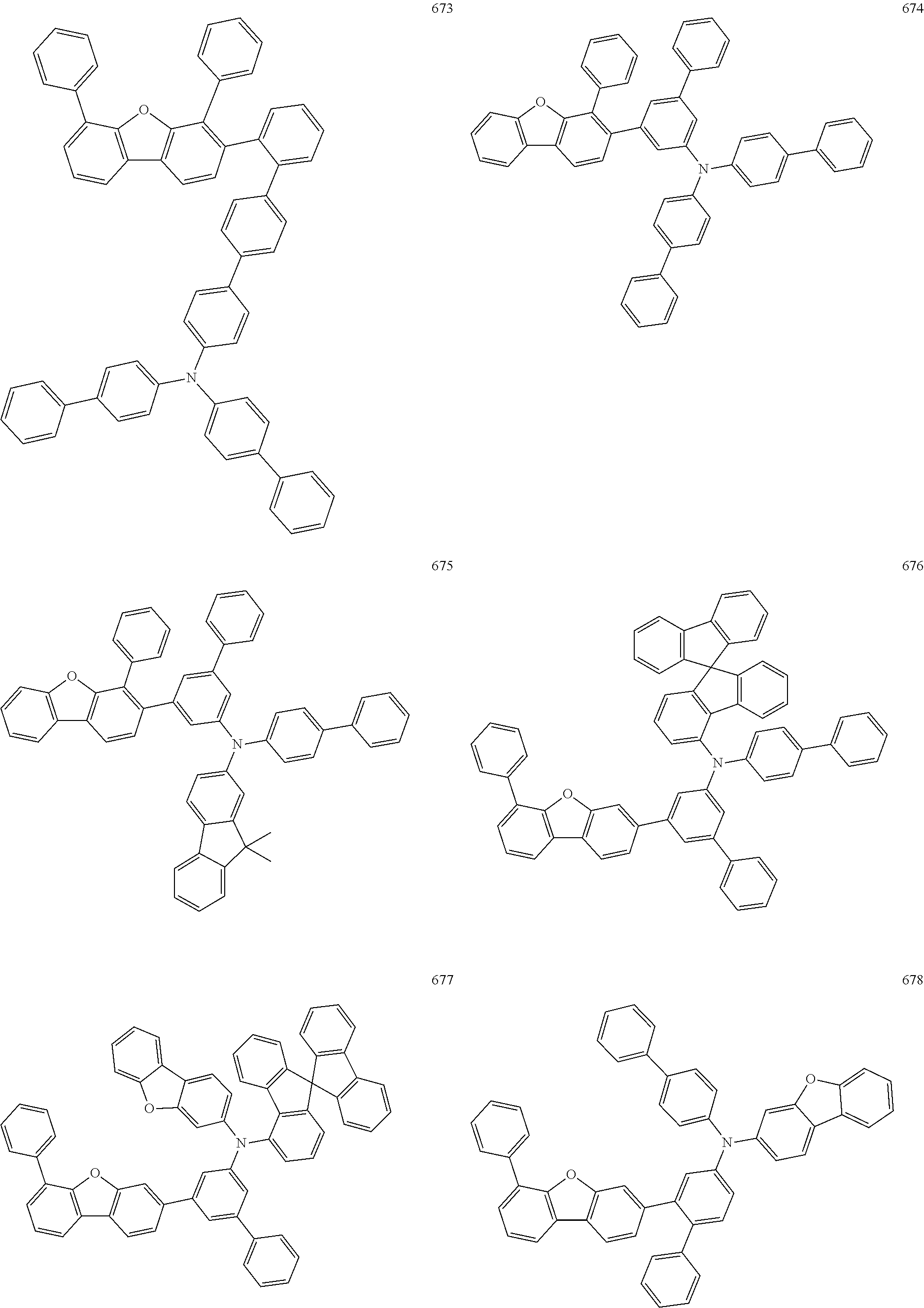

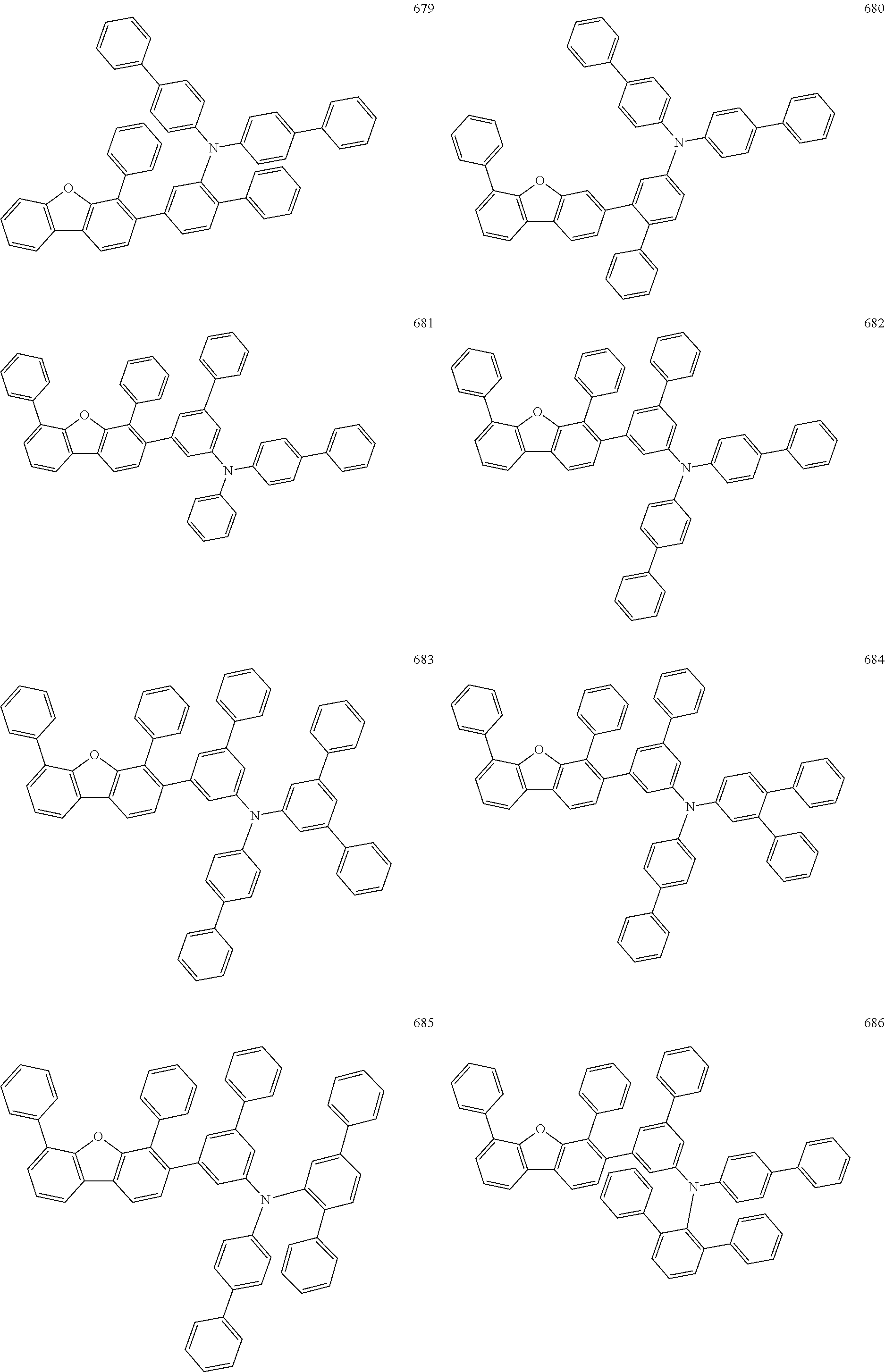

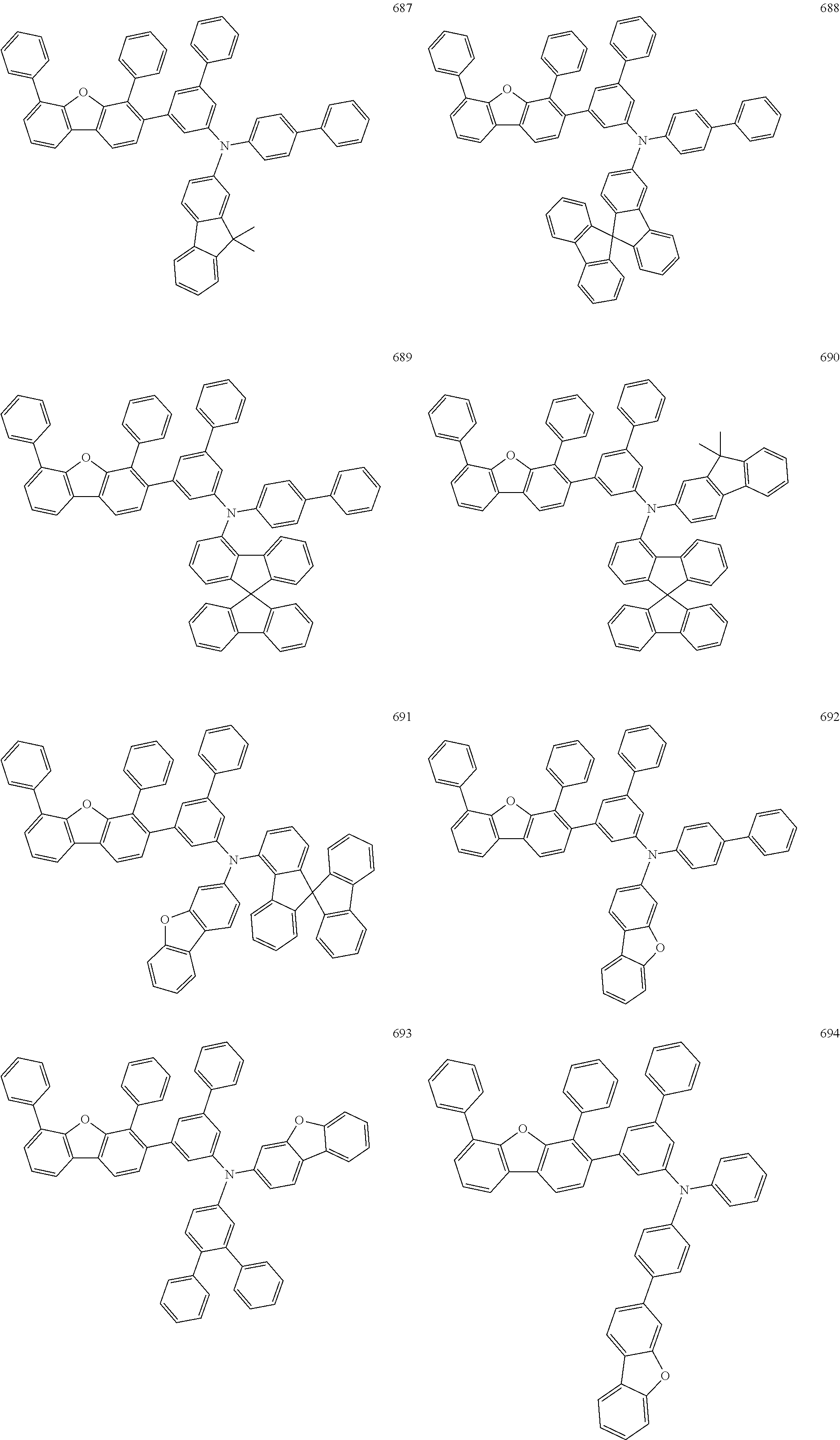

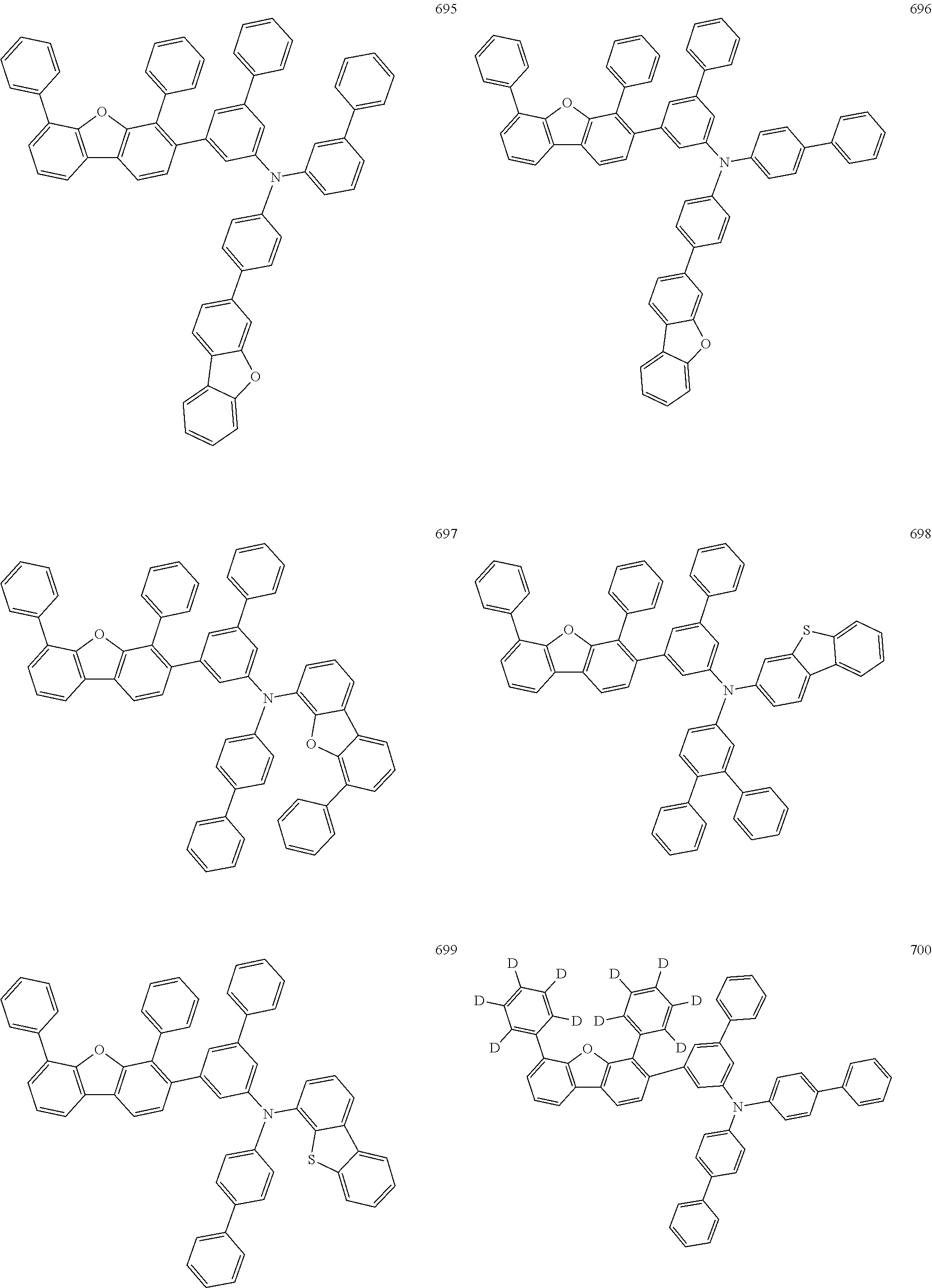

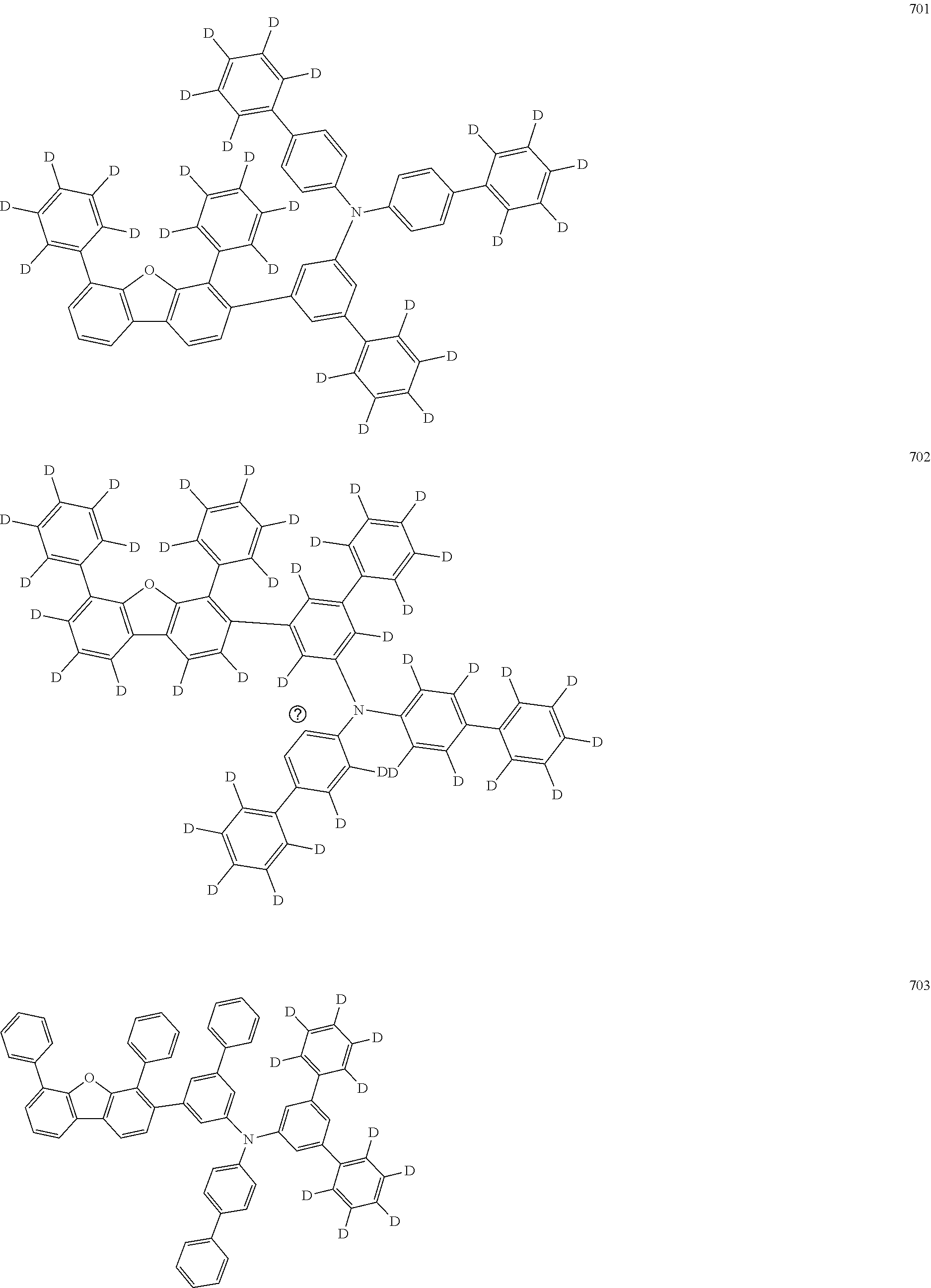

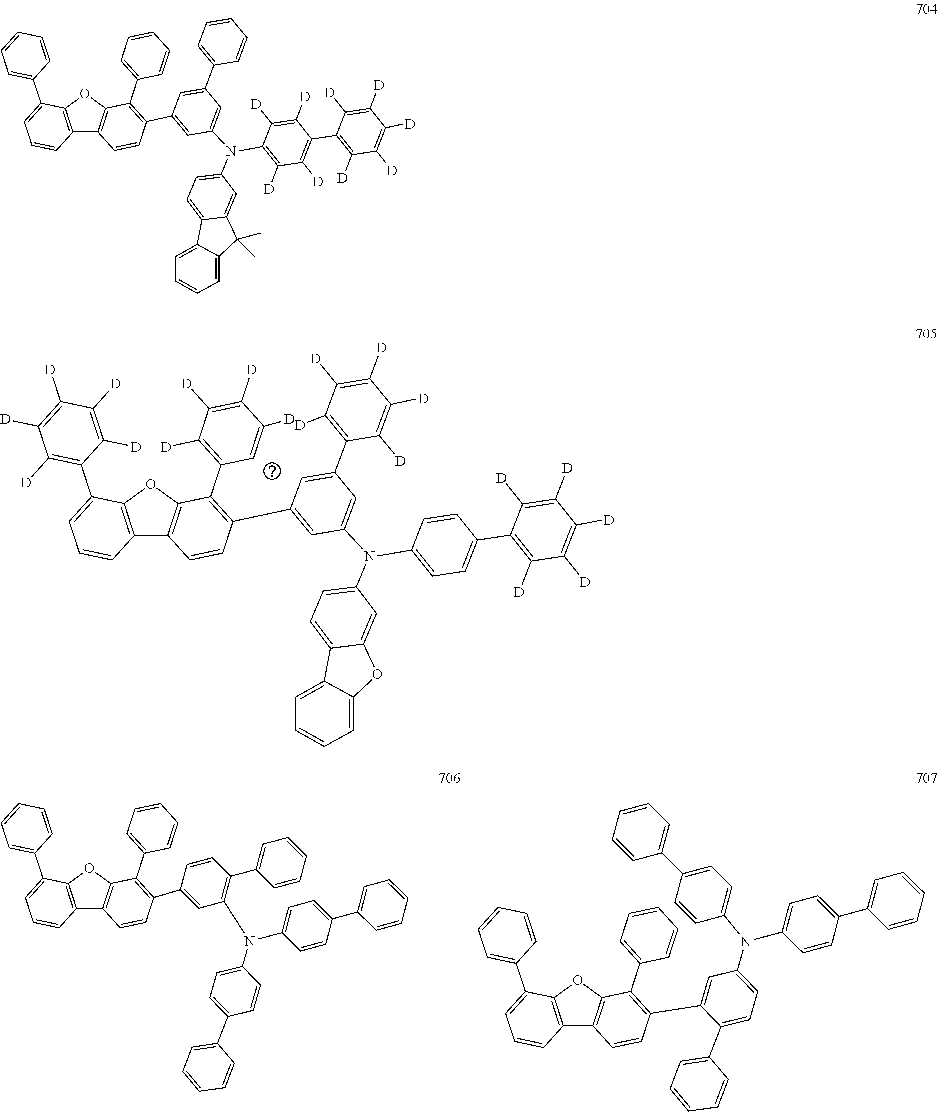

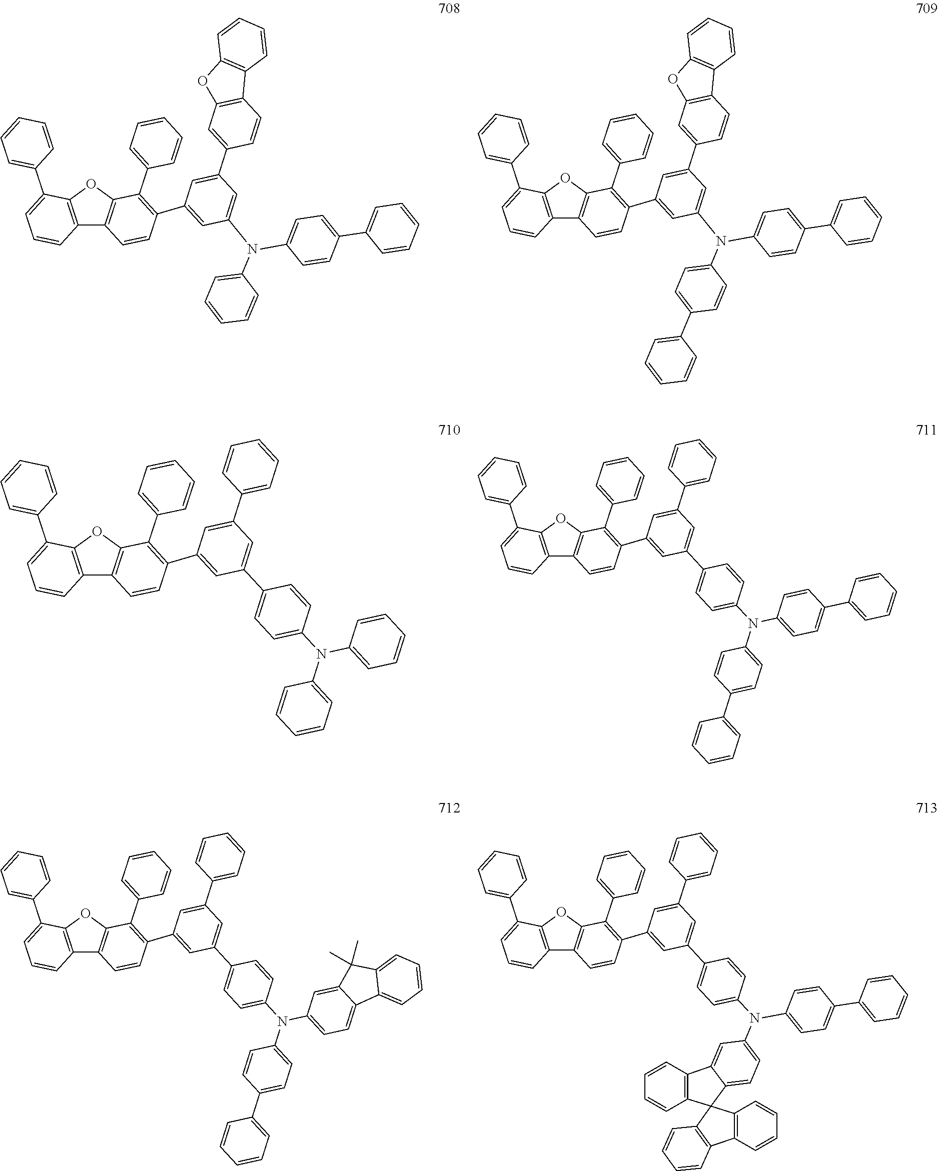

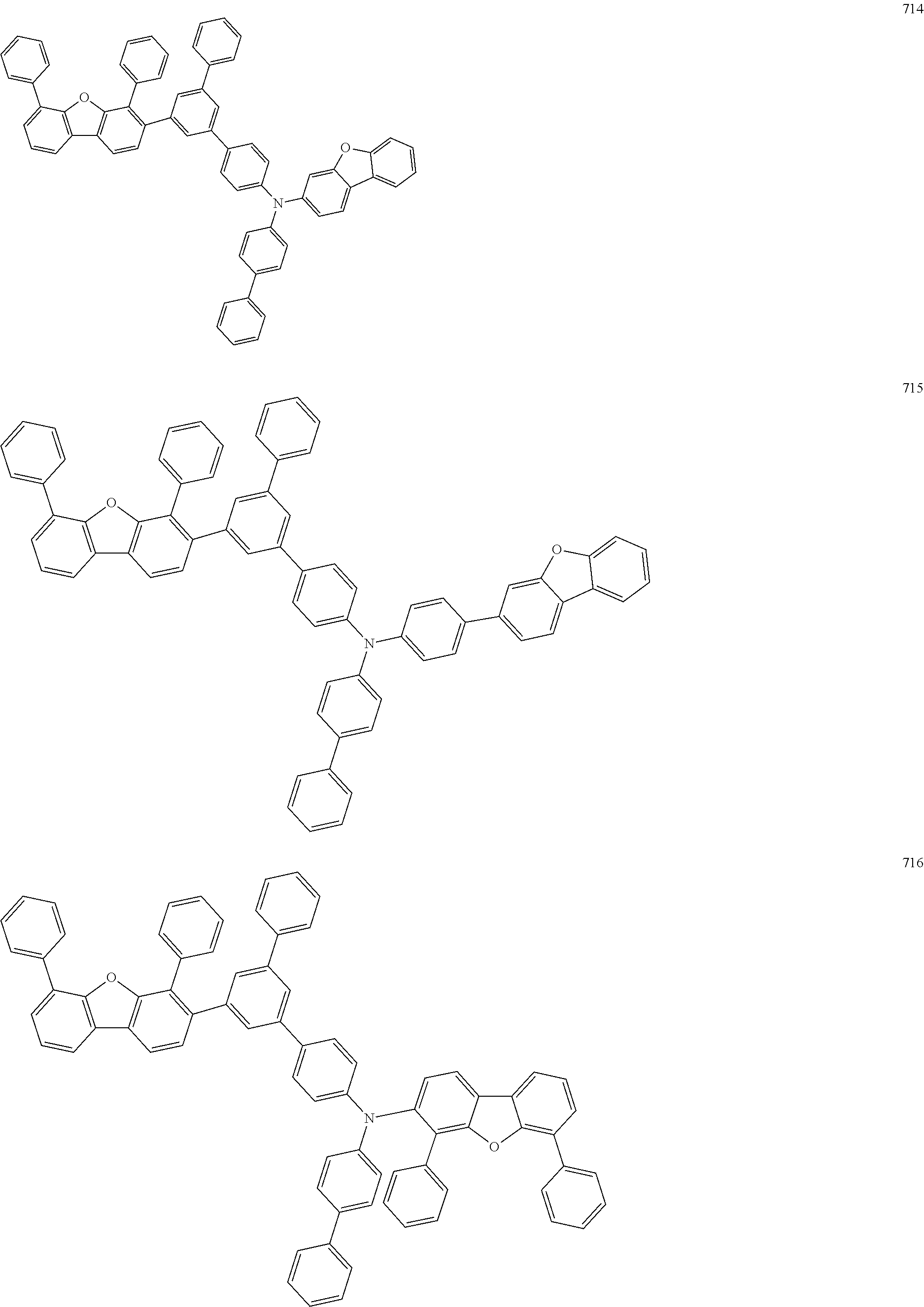

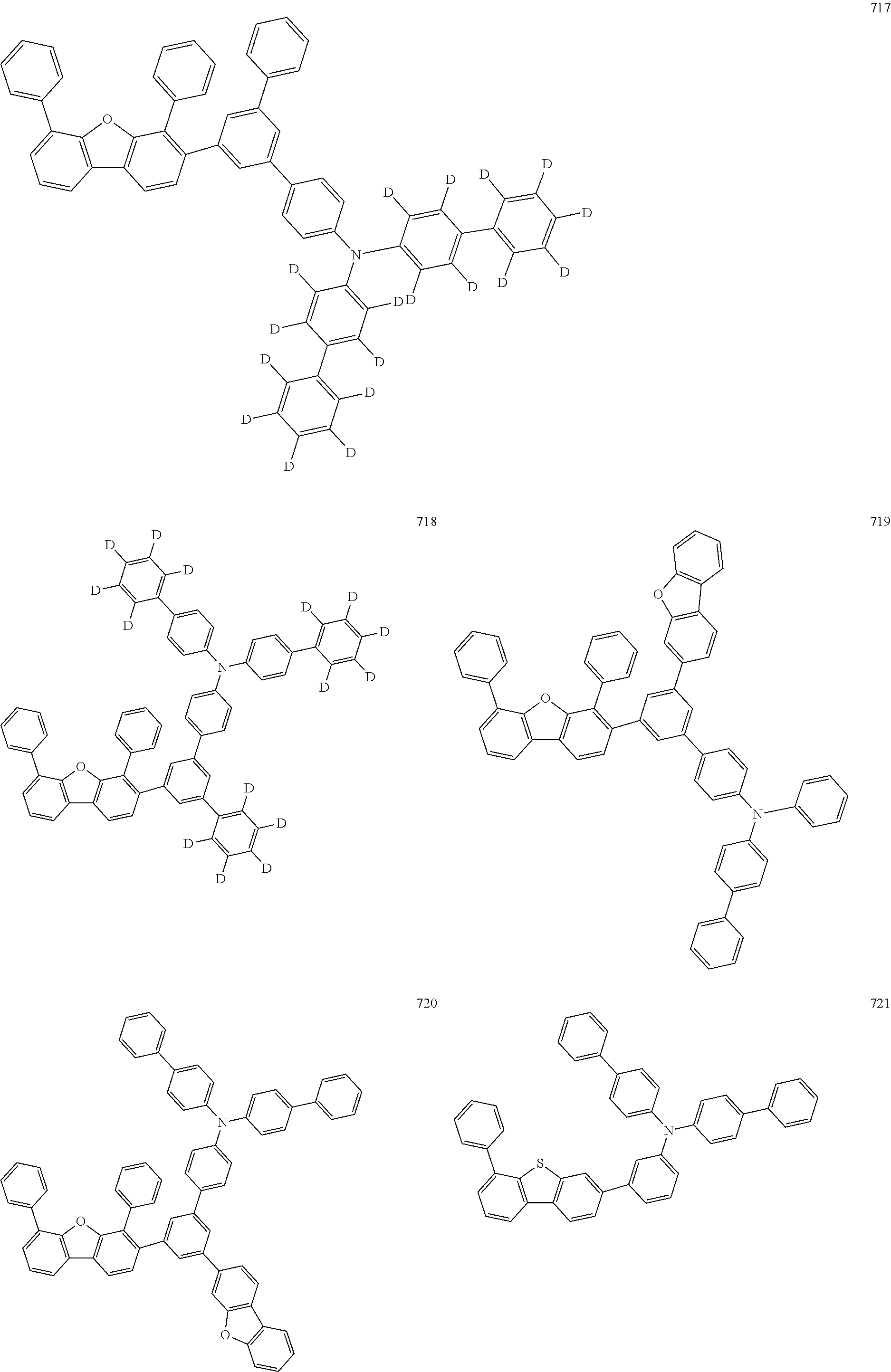

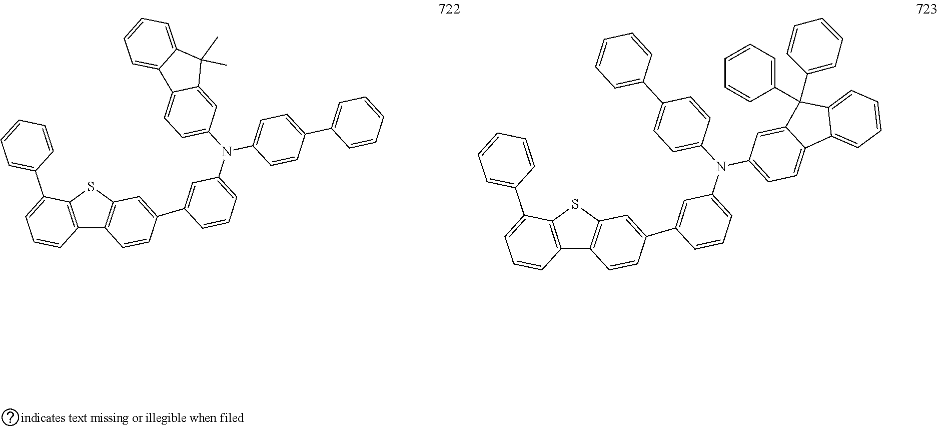 [SMILES: CC1(C)c2ccccc2-c2ccc(N(c3ccc(-c4ccccc4)cc3)c3cccc(-c4ccc5c(c4)sc4c(-c6ccccc6)cccc45)c3)cc21 ; c1ccc(-c2ccc(N(c3cccc(-c4ccc5c(c4)sc4c(-c6ccccc6)cccc45)c3)c3ccc4c(c3)C(c3ccccc3)(c3ccccc3)c3ccccc3-4)cc2)cc1]

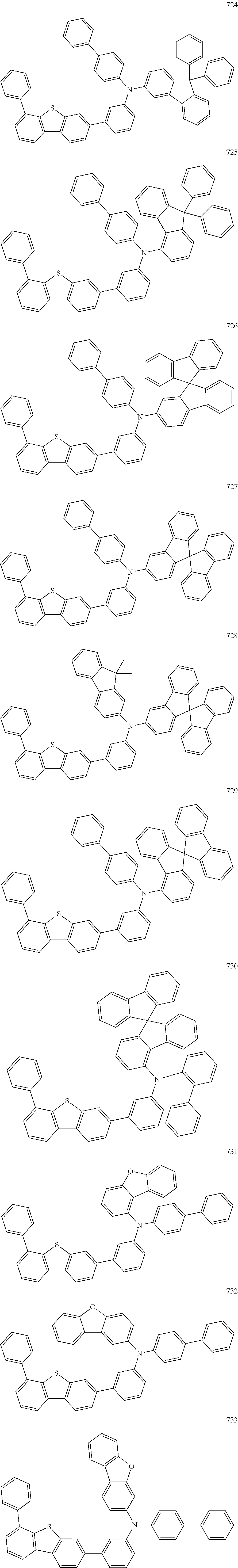

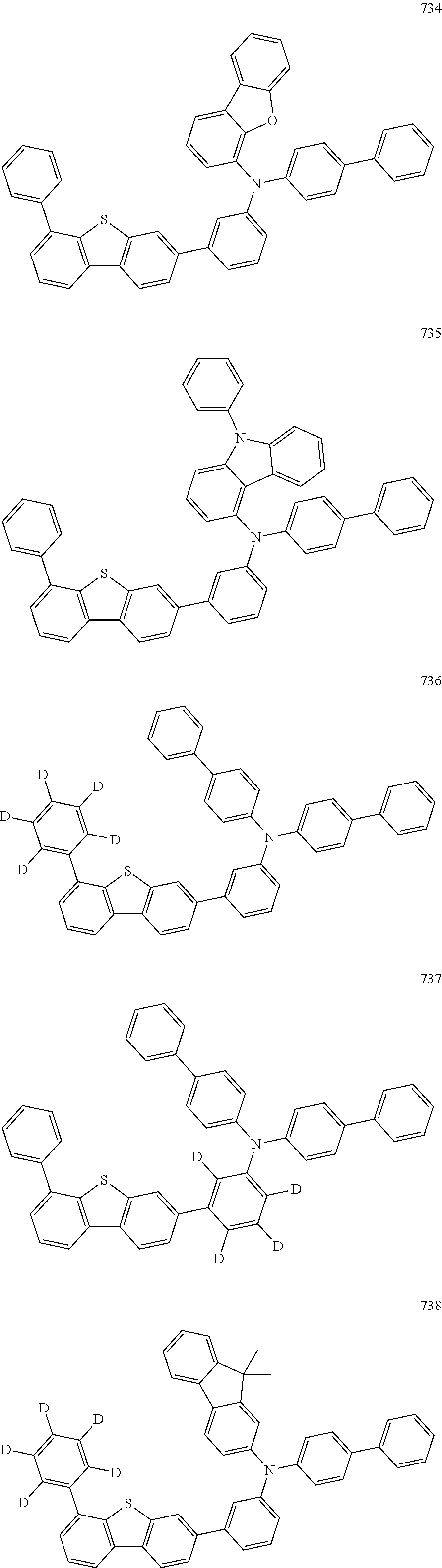

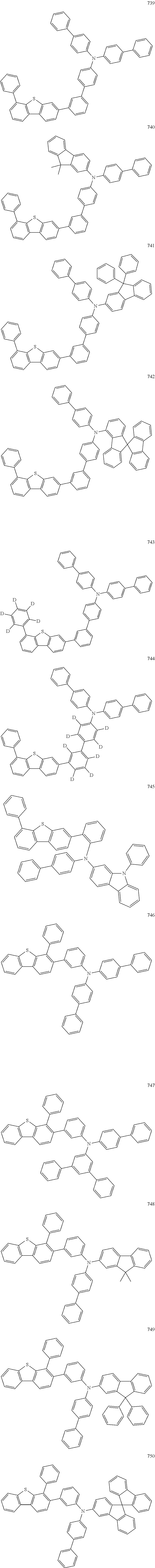

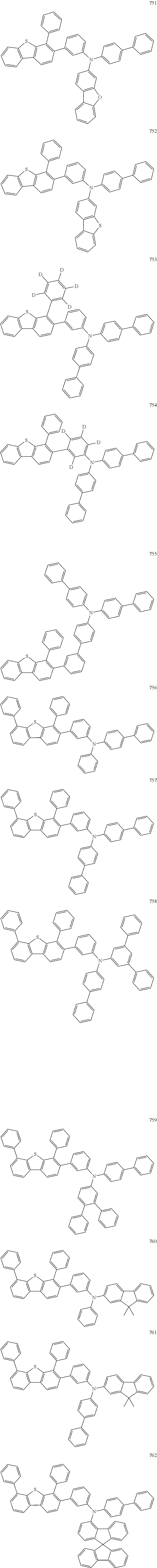

[chemical structure]

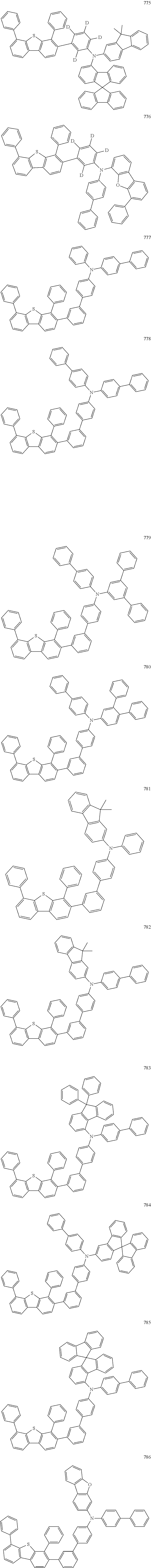

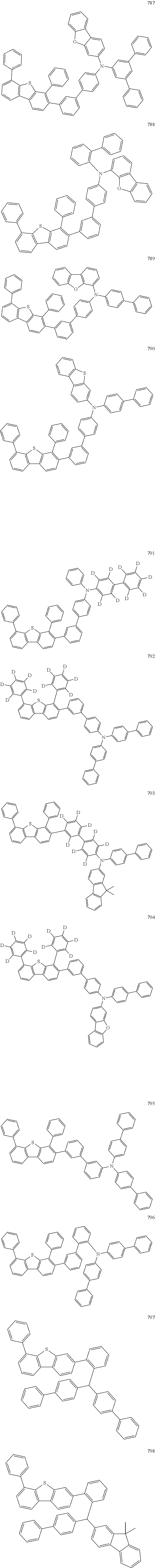

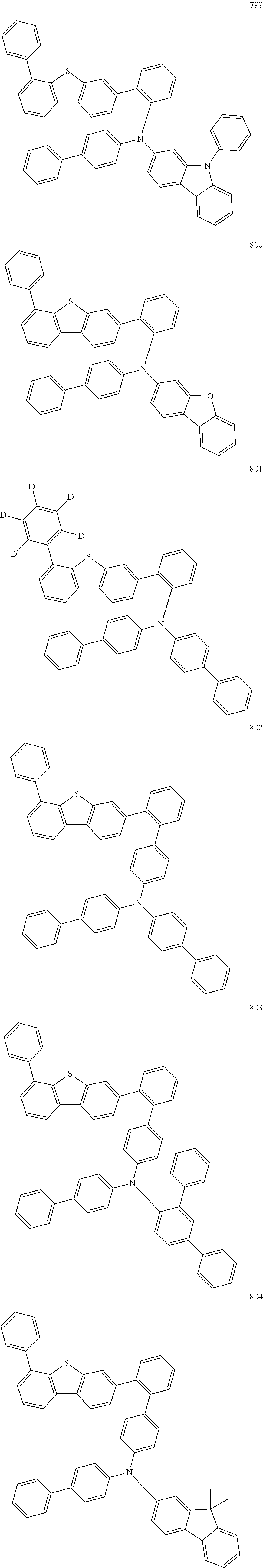

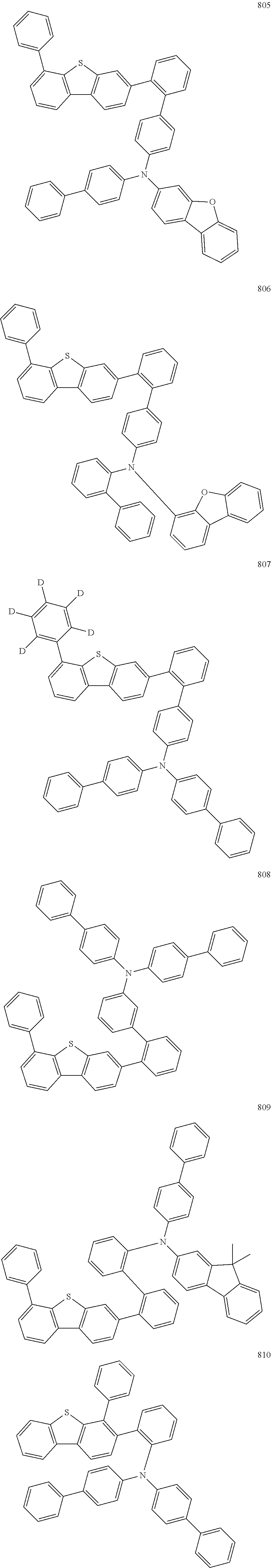

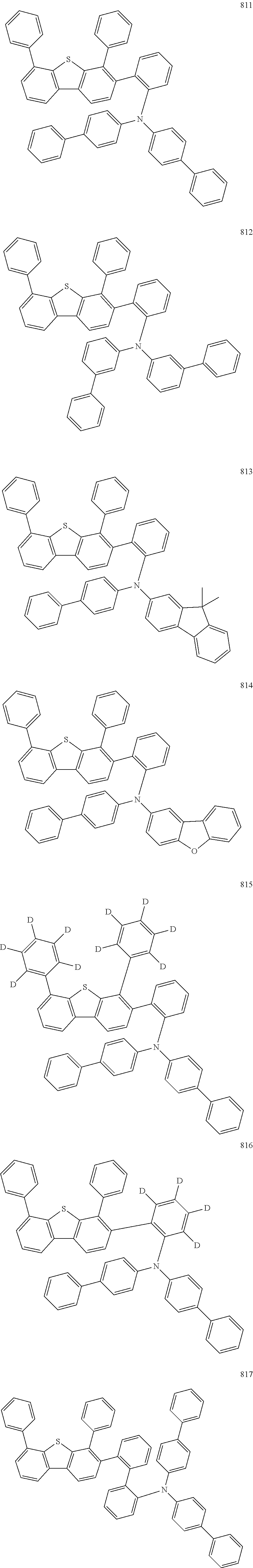

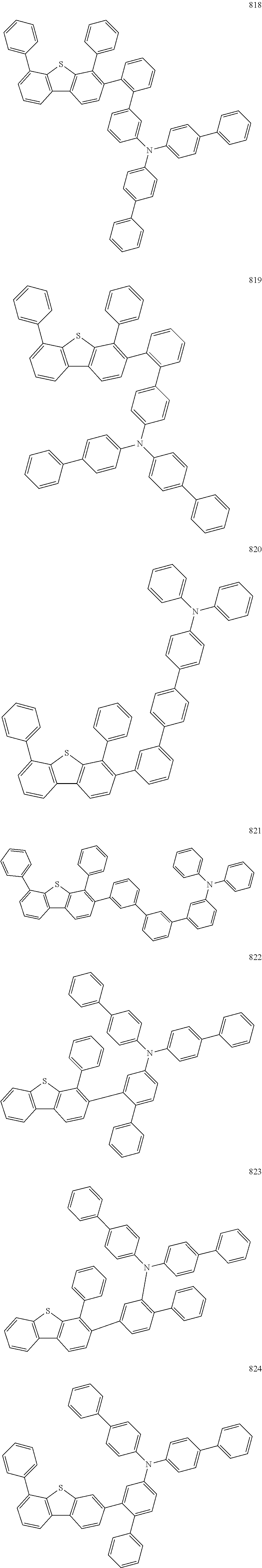

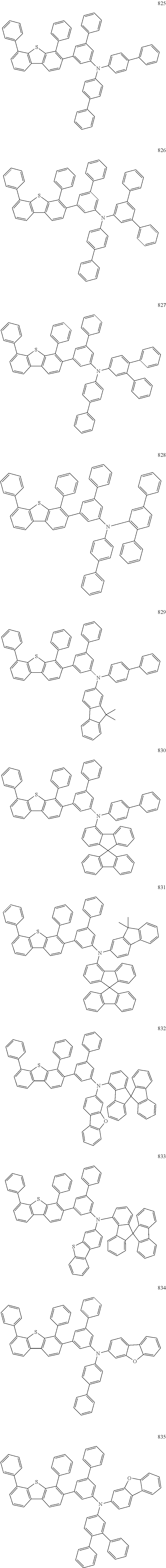

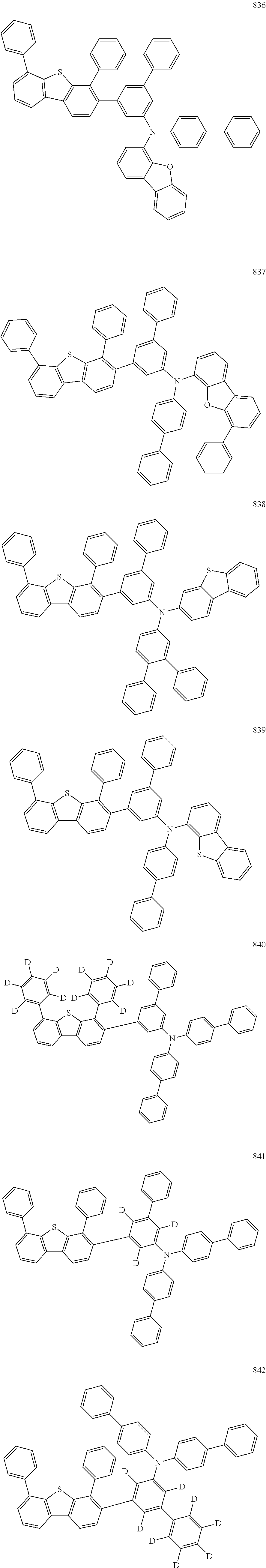

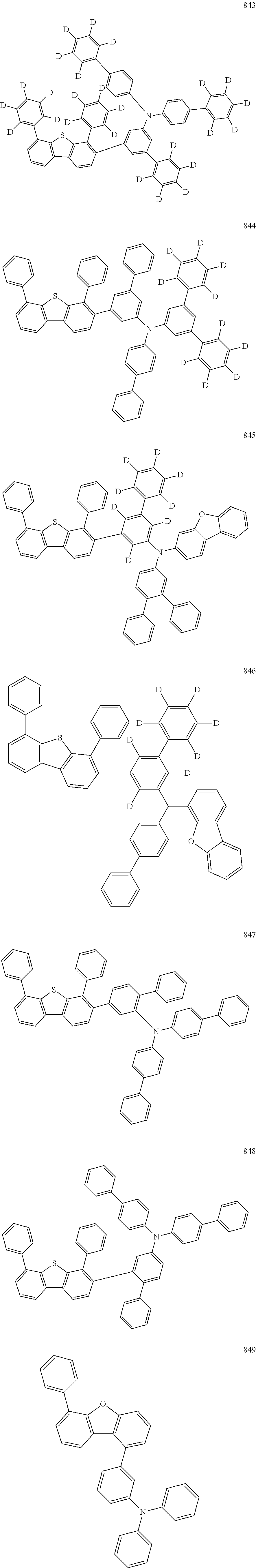

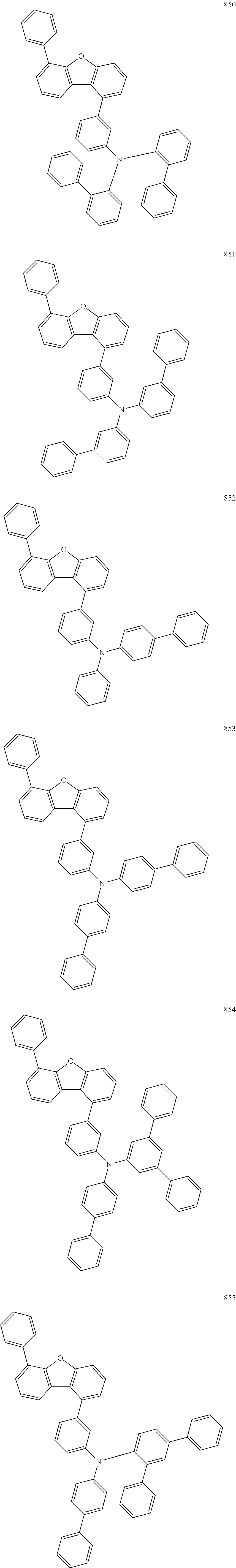

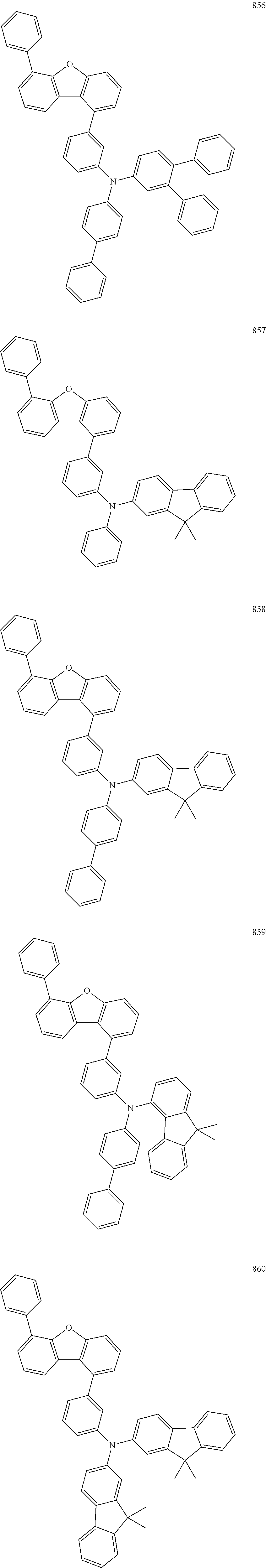 [SMILES: c1ccc(-c2ccc(N(c3cccc(-c4cccc5oc6c(-c7ccccc7)cccc6c45)c3)c3ccc(-c4ccccc4)c(-c4ccccc4)c3)cc2)cc1 ; CC1(C)c2ccccc2-c2ccc(N(c3ccccc3)c3cccc(-c4cccc5oc6c(-c7ccccc7)cccc6c45)c3)cc21 ; CC1(C)c2ccccc2-c2ccc(N(c3ccc(-c4ccccc4)cc3)c3cccc(-c4cccc5oc6c(-c7ccccc7)cccc6c45)c3)cc21 ; CC1(C)c2ccccc2-c2c(N(c3ccc(-c4ccccc4)cc3)c3cccc(-c4cccc5oc6c(-c7ccccc7)cccc6c45)c3)cccc21 ; CC1(C)c2ccccc2-c2ccc(N(c3cccc(-c4cccc5oc6c(-c7ccccc7)cccc6c45)c3)c3ccc4c(c3)C(C)(C)c3ccccc3-4)cc21]

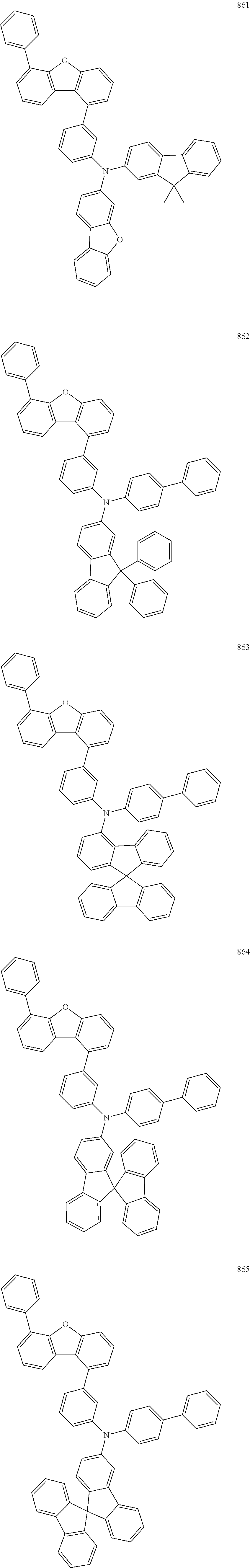

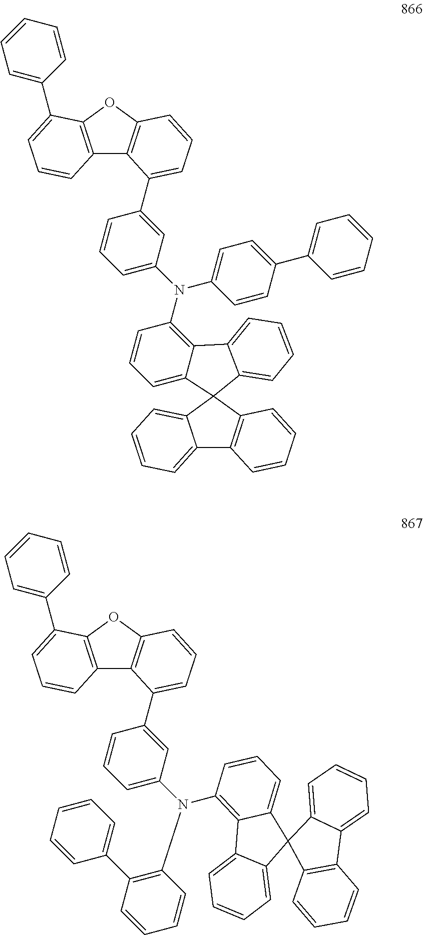 [SMILES: c1ccc(-c2ccc(N(c3cccc(-c4cccc5oc6c(-c7ccccc7)cccc6c45)c3)c3cccc4c3-c3ccccc3C43c4ccccc4-c4ccccc43)cc2)cc1 ; c1ccc(-c2ccccc2N(c2cccc(-c3cccc4oc5c(-c6ccccc6)cccc5c34)c2)c2cccc3c2-c2ccccc2C32c3ccccc3-c3ccccc32)cc1]

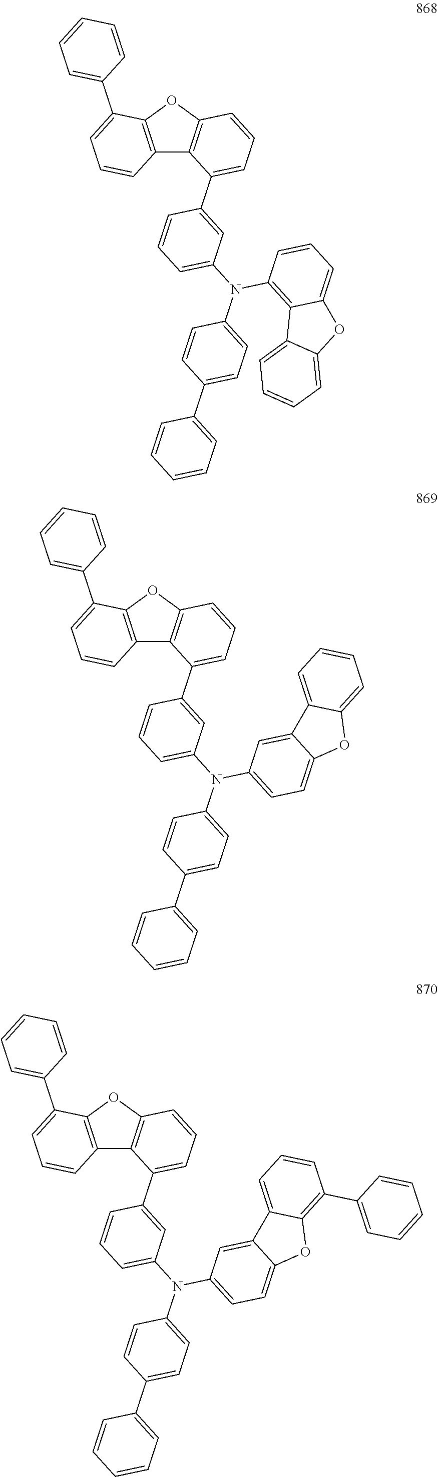 [SMILES: c1ccc(-c2ccc(N(c3cccc(-c4cccc5oc6c(-c7ccccc7)cccc6c45)c3)c3cccc4oc5ccccc5c34)cc2)cc1 ; c1ccc(-c2ccc(N(c3cccc(-c4cccc5oc6c(-c7ccccc7)cccc6c45)c3)c3ccc4oc5ccccc5c4c3)cc2)cc1 ; c1ccc(-c2ccc(N(c3cccc(-c4cccc5oc6c(-c7ccccc7)cccc6c45)c3)c3ccc4oc5c(-c6ccccc6)cccc5c4c3)cc2)cc1]

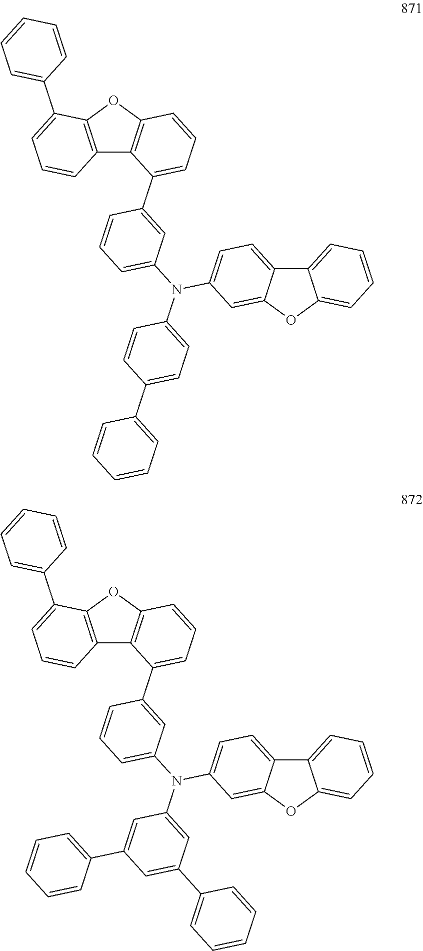 [SMILES: c1ccc(-c2ccc(N(c3cccc(-c4cccc5oc6c(-c7ccccc7)cccc6c45)c3)c3ccc4c(c3)oc3ccccc34)cc2)cc1 ; c1ccc(-c2cc(-c3ccccc3)cc(N(c3cccc(-c4cccc5oc6c(-c7ccccc7)cccc6c45)c3)c3ccc4c(c3)oc3ccccc34)c2)cc1]

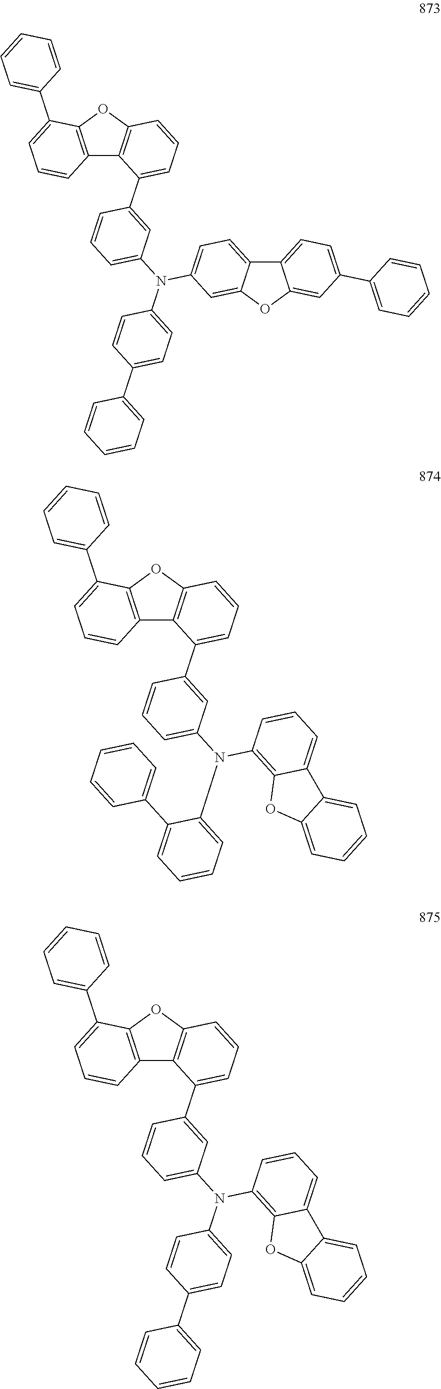

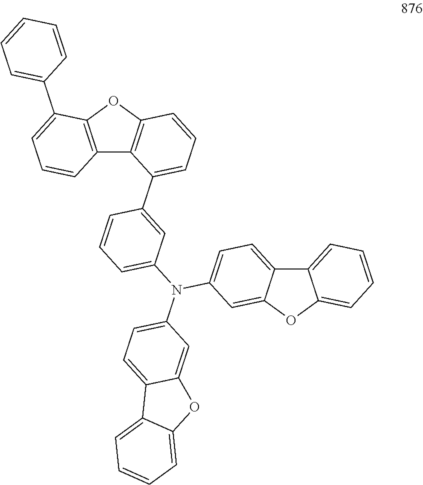 [SMILES: c1ccc(-c2cccc3c2oc2cccc(-c4cccc(N(c5ccc6c(c5)oc5ccccc56)c5ccc6c(c5)oc5ccccc56)c4)c23)cc1]

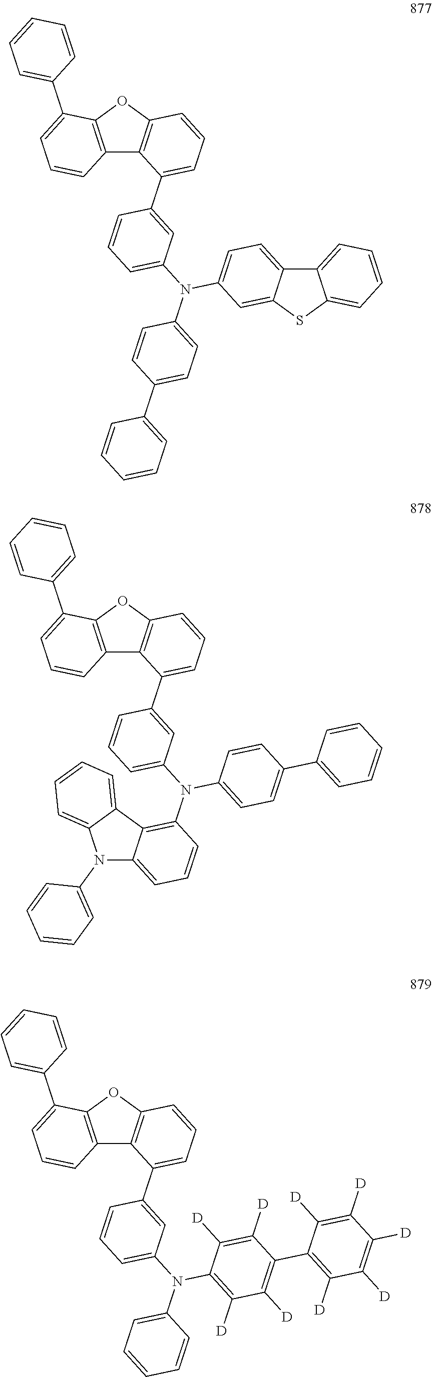 [SMILES: c1ccc(-c2ccc(N(c3cccc(-c4cccc5oc6c(-c7ccccc7)cccc6c45)c3)c3ccc4c(c3)sc3ccccc34)cc2)cc1 ; c1ccc(-c2ccc(N(c3cccc(-c4cccc5oc6c(-c7ccccc7)cccc6c45)c3)c3cccc4c3c3ccccc3n4-c3ccccc3)cc2)cc1 ; [2H]c1c([2H])c([2H])c(-c2c([2H])c([2H])c(N(c3ccccc3)c3cccc(-c4cccc5oc6c(-c7ccccc7)cccc6c45)c3)c([2H])c2[2H])c([2H])c1[2H]]

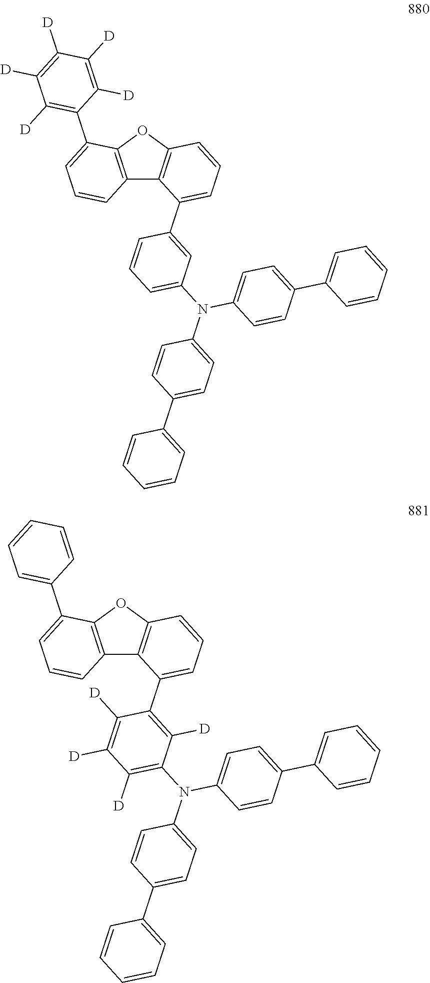 [SMILES: [2H]c1c([2H])c([2H])c(-c2cccc3c2oc2cccc(-c4cccc(N(c5ccc(-c6ccccc6)cc5)c5ccc(-c6ccccc6)cc5)c4)c23)c([2H])c1[2H] ; [2H]c1c([2H])c(-c2cccc3oc4c(-c5ccccc5)cccc4c23)c([2H])c(N(c2ccc(-c3ccccc3)cc2)c2ccc(-c3ccccc3)cc2)c1[2H]]

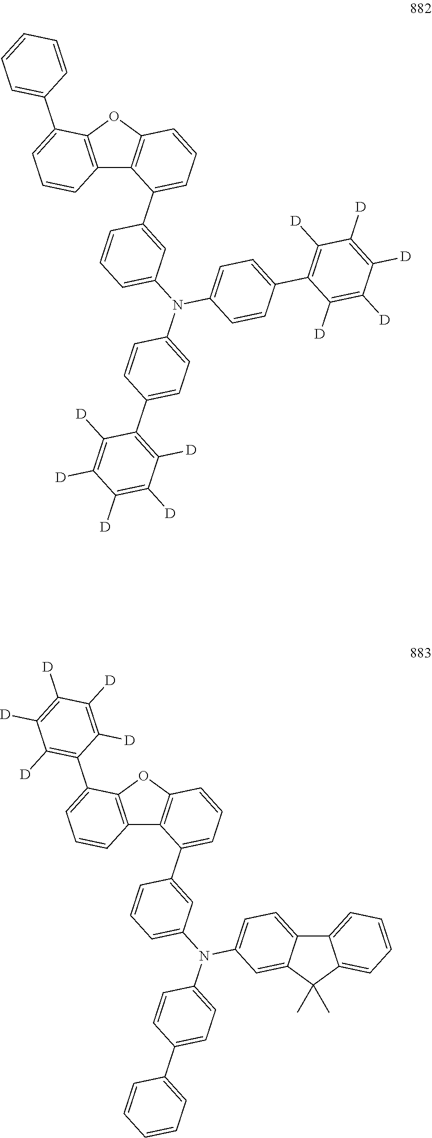 [SMILES: [2H]c1c([2H])c([2H])c(-c2ccc(N(c3ccc(-c4c([2H])c([2H])c([2H])c([2H])c4[2H])cc3)c3cccc(-c4cccc5oc6c(-c7ccccc7)cccc6c45)c3)cc2)c([2H])c1[2H] ; [2H]c1c([2H])c([2H])c(-c2cccc3c2oc2cccc(-c4cccc(N(c5ccc(-c6ccccc6)cc5)c5ccc6c(c5)C(C)(C)c5ccccc5-6)c4)c23)c([2H])c1[2H]]

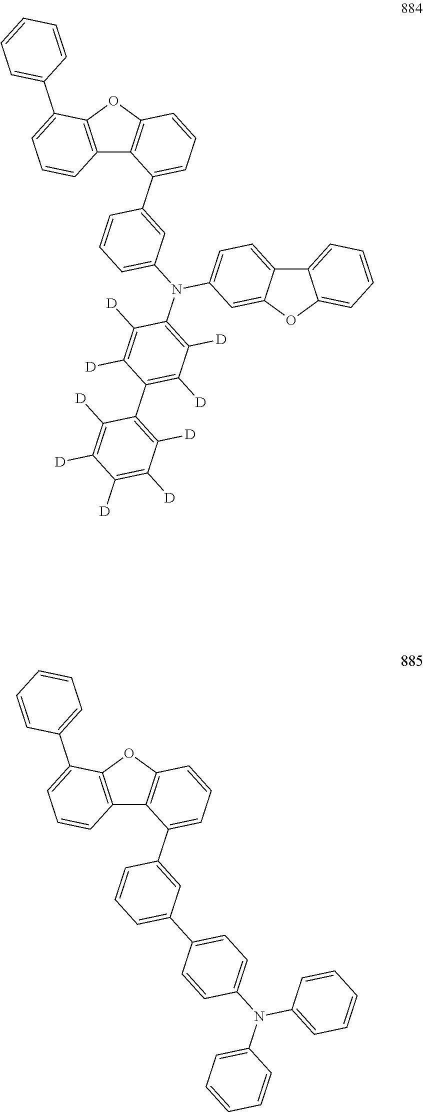 [SMILES: [2H]c1c([2H])c([2H])c(-c2c([2H])c([2H])c(N(c3cccc(-c4cccc5oc6c(-c7ccccc7)cccc6c45)c3)c3ccc4c(c3)oc3ccccc34)c([2H])c2[2H])c([2H])c1[2H] ; c1ccc(-c2cccc3c2oc2cccc(-c4cccc(-c5ccc(N(c6ccccc6)c6ccccc6)cc5)c4)c23)cc1]

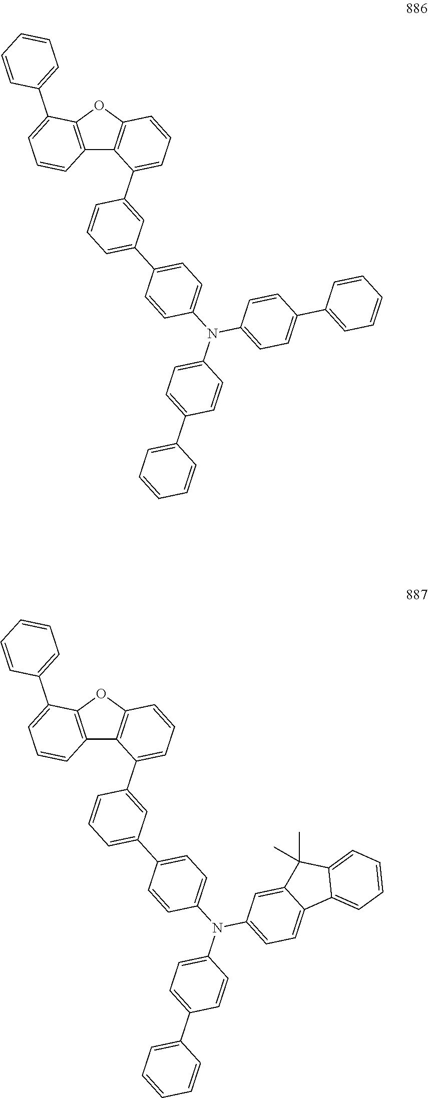 [SMILES: c1ccc(-c2ccc(N(c3ccc(-c4ccccc4)cc3)c3ccc(-c4cccc(-c5cccc6oc7c(-c8ccccc8)cccc7c56)c4)cc3)cc2)cc1 ; CC1(C)c2ccccc2-c2ccc(N(c3ccc(-c4ccccc4)cc3)c3ccc(-c4cccc(-c5cccc6oc7c(-c8ccccc8)cccc7c56)c4)cc3)cc21]

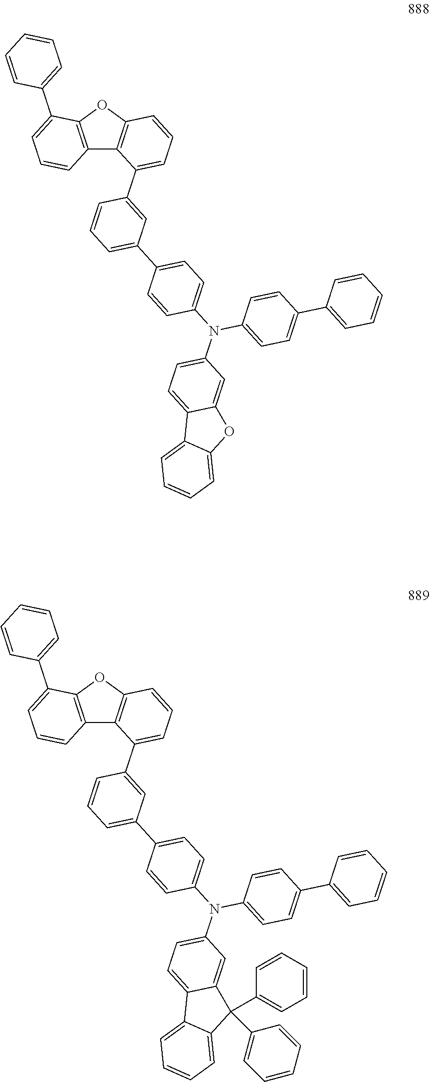 [SMILES: c1ccc(-c2ccc(N(c3ccc(-c4cccc(-c5cccc6oc7c(-c8ccccc8)cccc7c56)c4)cc3)c3ccc4c(c3)oc3ccccc34)cc2)cc1 ; c1ccc(-c2ccc(N(c3ccc(-c4cccc(-c5cccc6oc7c(-c8ccccc8)cccc7c56)c4)cc3)c3ccc4c(c3)C(c3ccccc3)(c3ccccc3)c3ccccc3-4)cc2)cc1]

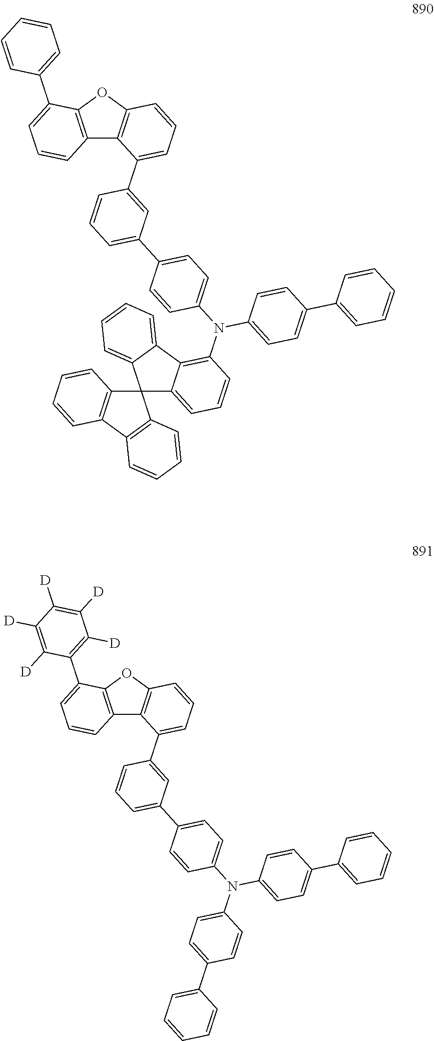 [SMILES: c1ccc(-c2ccc(N(c3ccc(-c4cccc(-c5cccc6oc7c(-c8ccccc8)cccc7c56)c4)cc3)c3cccc4c3-c3ccccc3C43c4ccccc4-c4ccccc43)cc2)cc1 ; [2H]c1c([2H])c([2H])c(-c2cccc3c2oc2cccc(-c4cccc(-c5ccc(N(c6ccc(-c7ccccc7)cc6)c6ccc(-c7ccccc7)cc6)cc5)c4)c23)c([2H])c1[2H]]

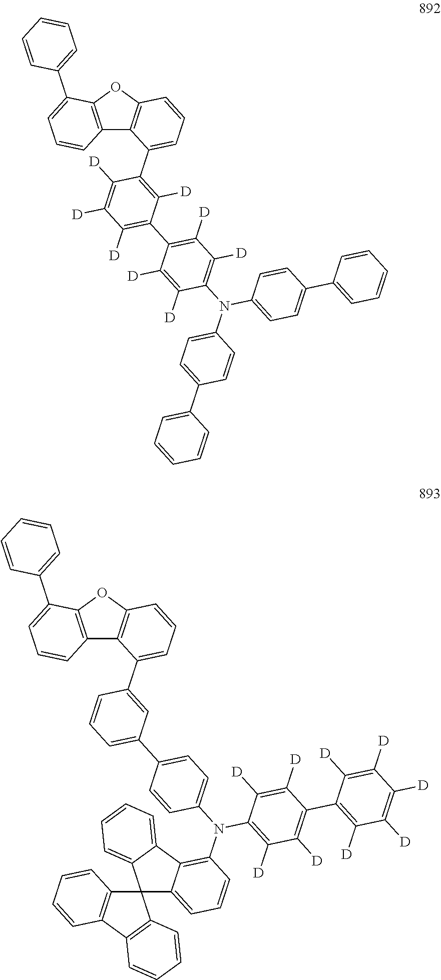 [SMILES: [2H]c1c([2H])c(-c2c([2H])c([2H])c(N(c3ccc(-c4ccccc4)cc3)c3ccc(-c4ccccc4)cc3)c([2H])c2[2H])c([2H])c(-c2cccc3oc4c(-c5ccccc5)cccc4c23)c1[2H] ; [2H]c1c([2H])c([2H])c(-c2c([2H])c([2H])c(N(c3ccc(-c4cccc(-c5cccc6oc7c(-c8ccccc8)cccc7c56)c4)cc3)c3cccc4c3-c3ccccc3C43c4ccccc4-c4ccccc43)c([2H])c2[2H])c([2H])c1[2H]]

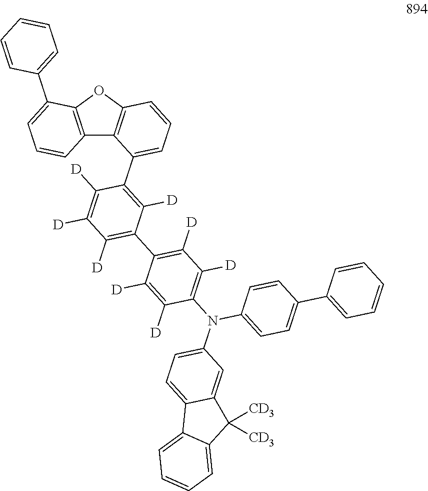 [SMILES: [2H]c1c([2H])c(-c2c([2H])c([2H])c(N(c3ccc(-c4ccccc4)cc3)c3ccc4c(c3)C(C([2H])([2H])[2H])(C([2H])([2H])[2H])c3ccccc3-4)c([2H])c2[2H])c([2H])c(-c2cccc3oc4c(-c5ccccc5)cccc4c23)c1[2H]]

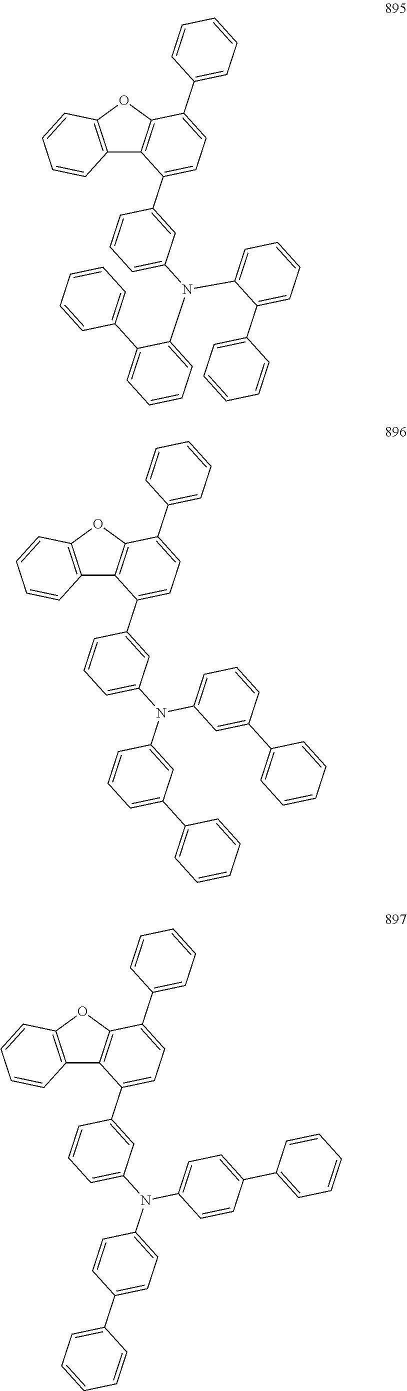

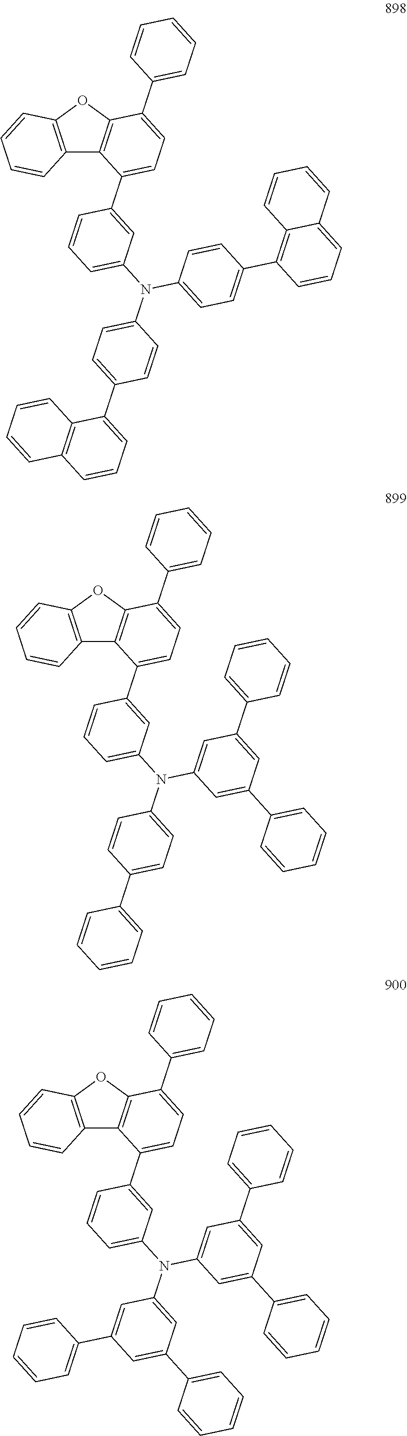 [SMILES: c1ccc(-c2ccc(-c3cccc(N(c4ccc(-c5cccc6ccccc56)cc4)c4ccc(-c5cccc6ccccc56)cc4)c3)c3c2oc2ccccc23)cc1 ; c1ccc(-c2ccc(N(c3cc(-c4ccccc4)cc(-c4ccccc4)c3)c3cccc(-c4ccc(-c5ccccc5)c5oc6ccccc6c45)c3)cc2)cc1 ; c1ccc(-c2cc(-c3ccccc3)cc(N(c3cc(-c4ccccc4)cc(-c4ccccc4)c3)c3cccc(-c4ccc(-c5ccccc5)c5oc6ccccc6c45)c3)c2)cc1]

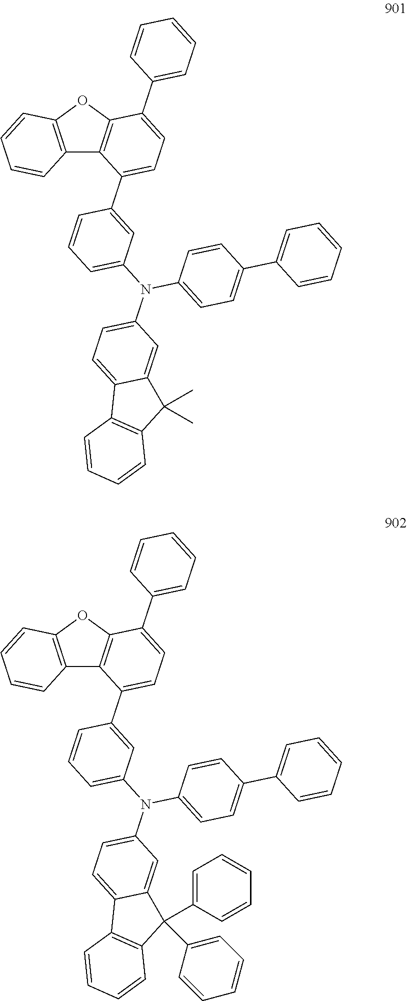 [SMILES: CC1(C)c2ccccc2-c2ccc(N(c3ccc(-c4ccccc4)cc3)c3cccc(-c4ccc(-c5ccccc5)c5oc6ccccc6c45)c3)cc21 ; c1ccc(-c2ccc(N(c3cccc(-c4ccc(-c5ccccc5)c5oc6ccccc6c45)c3)c3ccc4c(c3)C(c3ccccc3)(c3ccccc3)c3ccccc3-4)cc2)cc1]

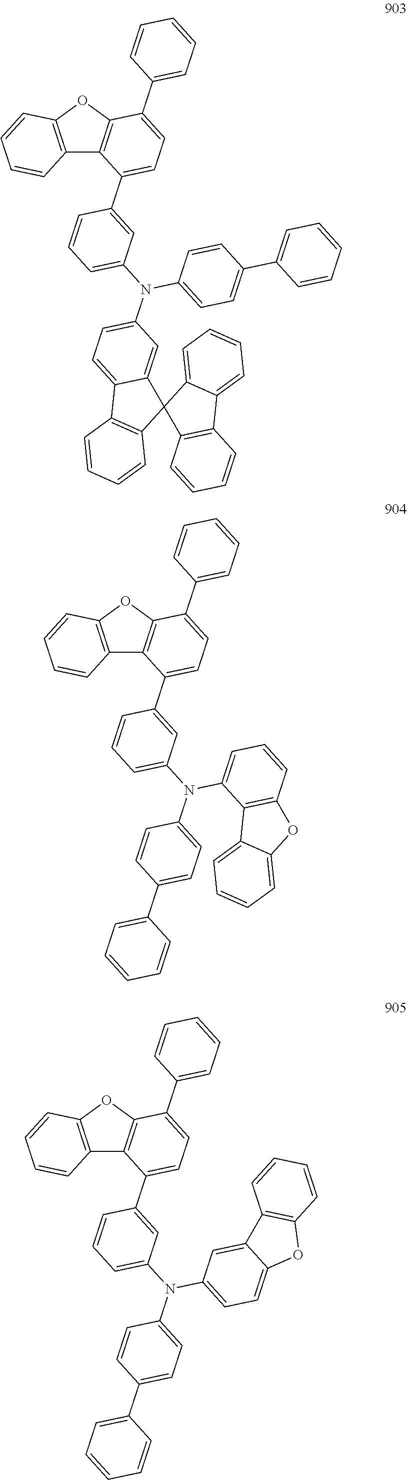

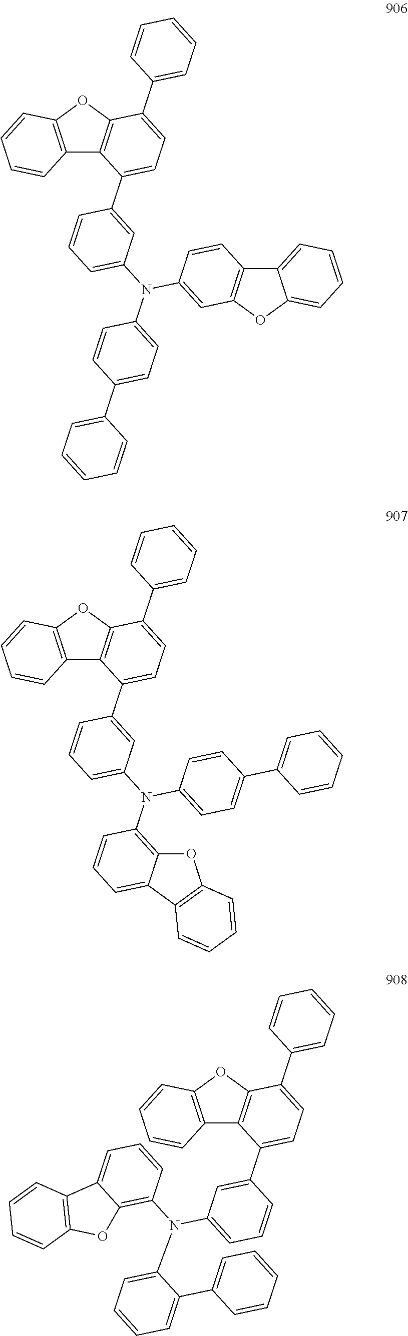 [SMILES: c1ccc(-c2ccc(N(c3cccc(-c4ccc(-c5ccccc5)c5oc6ccccc6c45)c3)c3ccc4c(c3)oc3ccccc34)cc2)cc1 ; c1ccc(-c2ccc(N(c3cccc(-c4ccc(-c5ccccc5)c5oc6ccccc6c45)c3)c3cccc4c3oc3ccccc34)cc2)cc1 ; c1ccc(-c2ccccc2N(c2cccc(-c3ccc(-c4ccccc4)c4oc5ccccc5c34)c2)c2cccc3c2oc2ccccc23)cc1]

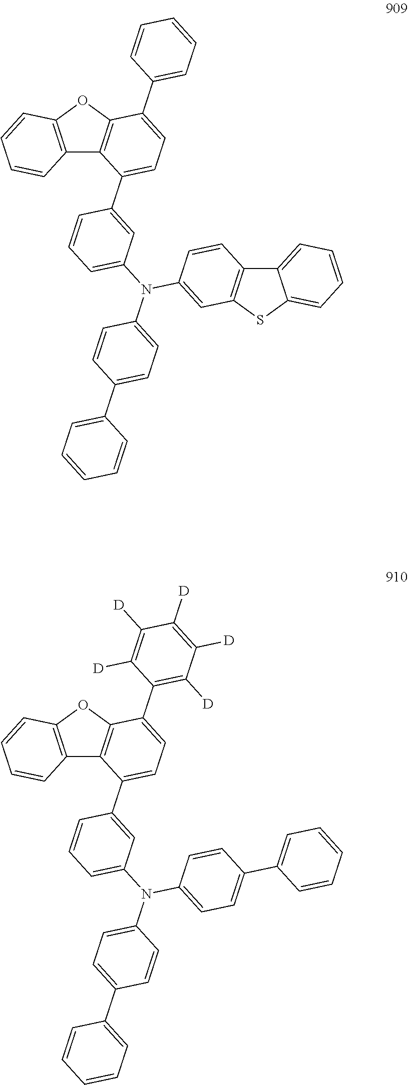 [SMILES: c1ccc(-c2ccc(N(c3cccc(-c4ccc(-c5ccccc5)c5oc6ccccc6c45)c3)c3ccc4c(c3)sc3ccccc34)cc2)cc1 ; [2H]c1c([2H])c([2H])c(-c2ccc(-c3cccc(N(c4ccc(-c5ccccc5)cc4)c4ccc(-c5ccccc5)cc4)c3)c3c2oc2ccccc23)c([2H])c1[2H]]

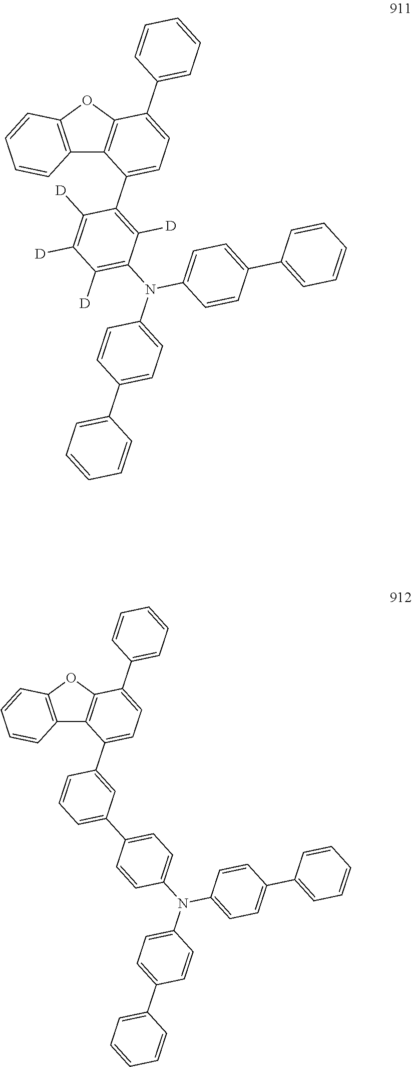 [SMILES: [2H]c1c([2H])c(-c2ccc(-c3ccccc3)c3oc4ccccc4c23)c([2H])c(N(c2ccc(-c3ccccc3)cc2)c2ccc(-c3ccccc3)cc2)c1[2H] ; c1ccc(-c2ccc(N(c3ccc(-c4ccccc4)cc3)c3ccc(-c4cccc(-c5ccc(-c6ccccc6)c6oc7ccccc7c56)c4)cc3)cc2)cc1]

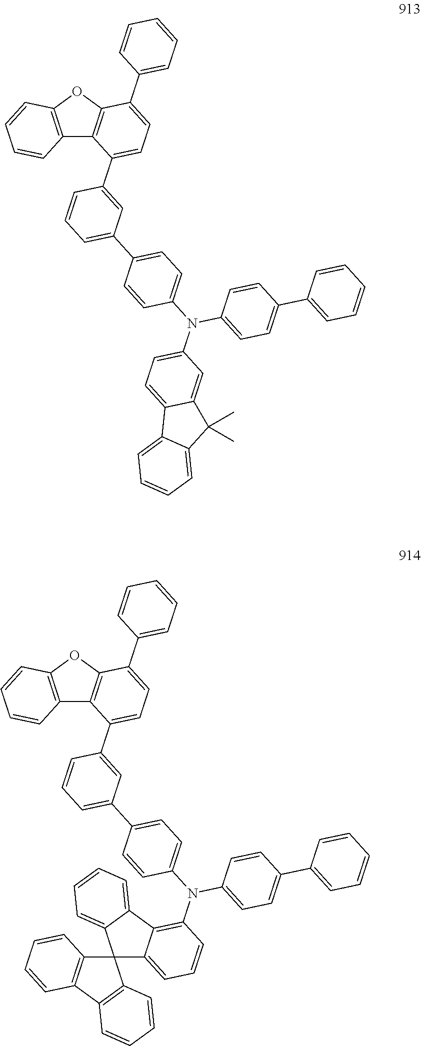 [SMILES: CC1(C)c2ccccc2-c2ccc(N(c3ccc(-c4ccccc4)cc3)c3ccc(-c4cccc(-c5ccc(-c6ccccc6)c6oc7ccccc7c56)c4)cc3)cc21 ; c1ccc(-c2ccc(N(c3ccc(-c4cccc(-c5ccc(-c6ccccc6)c6oc7ccccc7c56)c4)cc3)c3cccc4c3-c3ccccc3C43c4ccccc4-c4ccccc43)cc2)cc1]

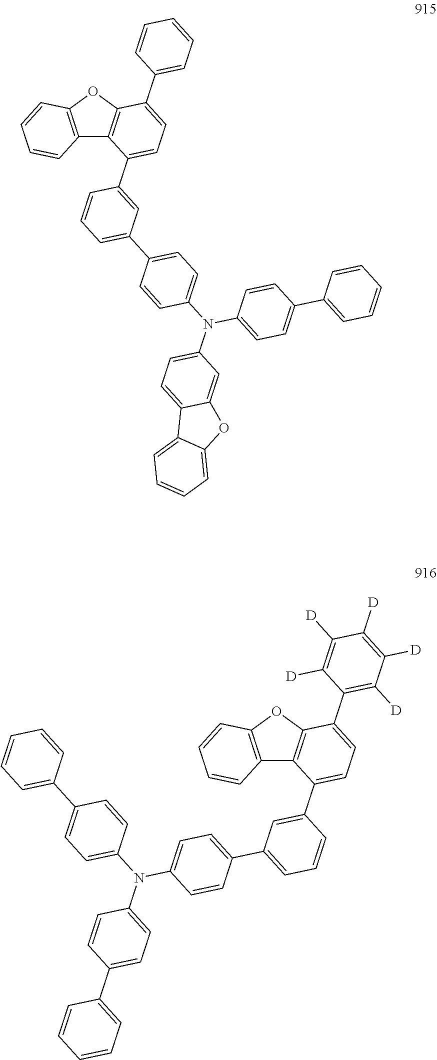 [SMILES: c1ccc(-c2ccc(N(c3ccc(-c4cccc(-c5ccc(-c6ccccc6)c6oc7ccccc7c56)c4)cc3)c3ccc4c(c3)oc3ccccc34)cc2)cc1 ; [2H]c1c([2H])c([2H])c(-c2ccc(-c3cccc(-c4ccc(N(c5ccc(-c6ccccc6)cc5)c5ccc(-c6ccccc6)cc5)cc4)c3)c3c2oc2ccccc23)c([2H])c1[2H]]

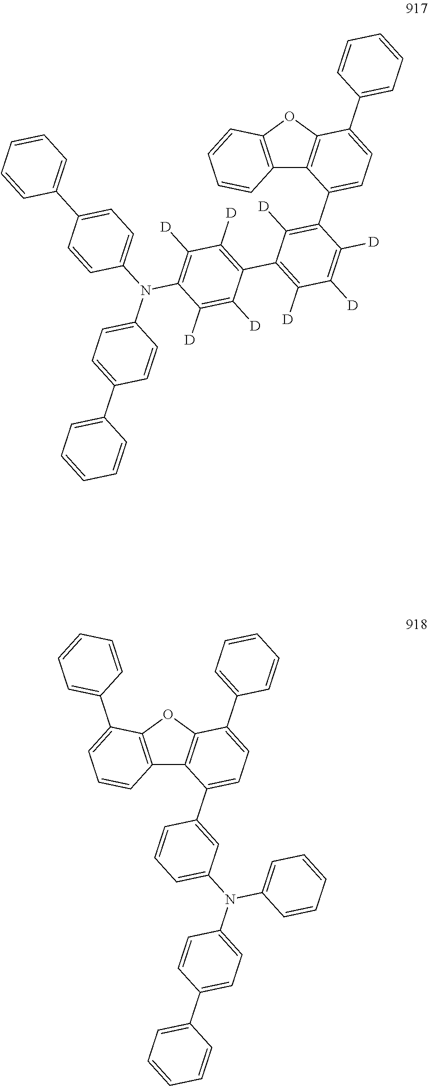 [SMILES: [2H]c1c([2H])c(-c2c([2H])c([2H])c(N(c3ccc(-c4ccccc4)cc3)c3ccc(-c4ccccc4)cc3)c([2H])c2[2H])c([2H])c(-c2ccc(-c3ccccc3)c3oc4ccccc4c23)c1[2H] ; c1ccc(-c2ccc(N(c3ccccc3)c3cccc(-c4ccc(-c5ccccc5)c5oc6c(-c7ccccc7)cccc6c45)c3)cc2)cc1]

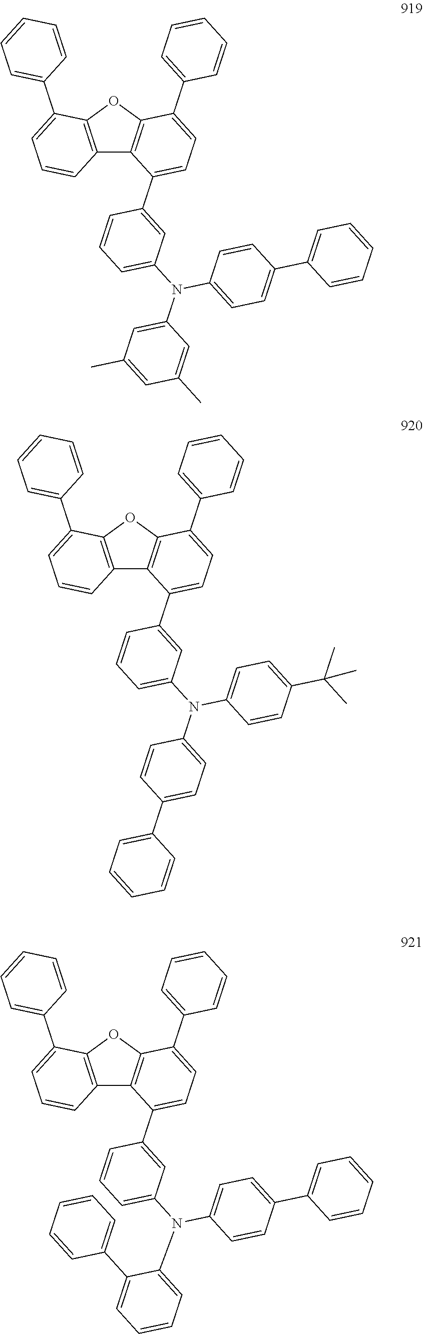

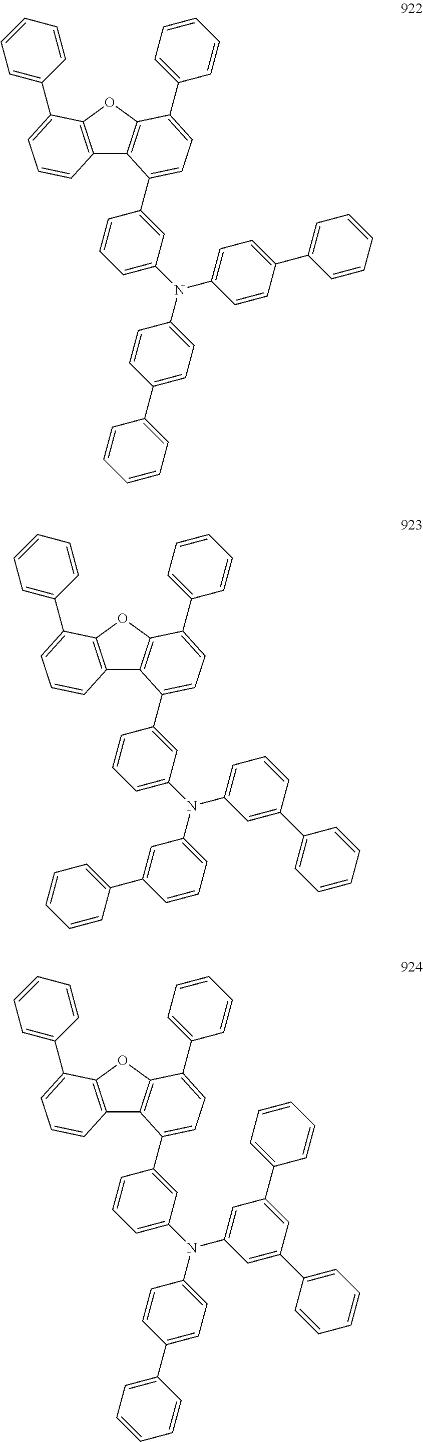 [SMILES: c1ccc(-c2ccc(N(c3ccc(-c4ccccc4)cc3)c3cccc(-c4ccc(-c5ccccc5)c5oc6c(-c7ccccc7)cccc6c45)c3)cc2)cc1 ; c1ccc(-c2cccc(N(c3cccc(-c4ccccc4)c3)c3cccc(-c4ccc(-c5ccccc5)c5oc6c(-c7ccccc7)cccc6c45)c3)c2)cc1 ; c1ccc(-c2ccc(N(c3cc(-c4ccccc4)cc(-c4ccccc4)c3)c3cccc(-c4ccc(-c5ccccc5)c5oc6c(-c7ccccc7)cccc6c45)c3)cc2)cc1]

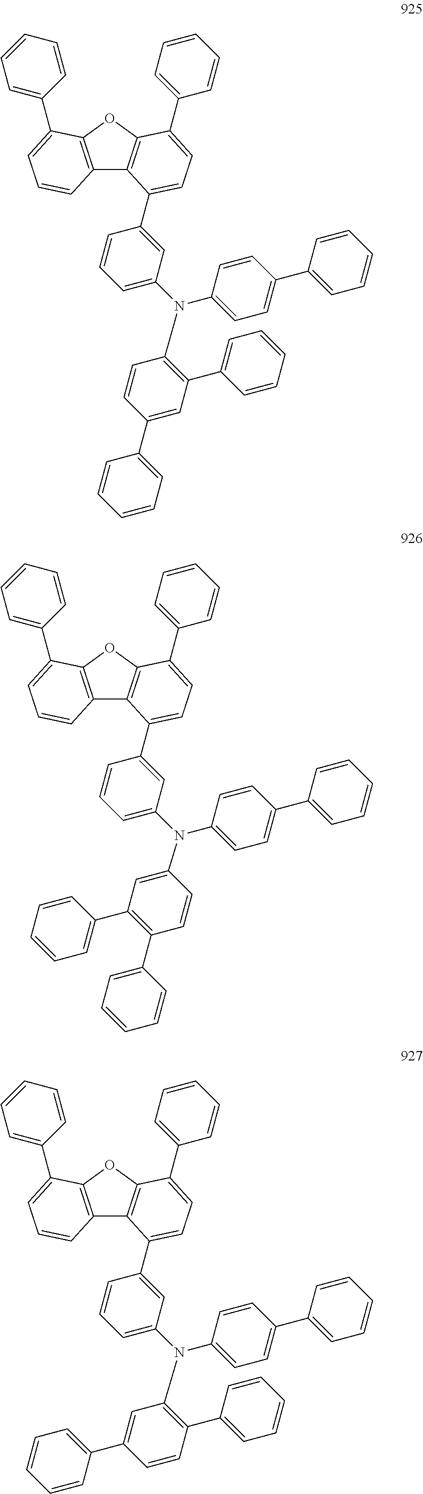 [SMILES: c1ccc(-c2ccc(N(c3cccc(-c4ccc(-c5ccccc5)c5oc6c(-c7ccccc7)cccc6c45)c3)c3ccc(-c4ccccc4)cc3-c3ccccc3)cc2)cc1 ; c1ccc(-c2ccc(N(c3cccc(-c4ccc(-c5ccccc5)c5oc6c(-c7ccccc7)cccc6c45)c3)c3ccc(-c4ccccc4)c(-c4ccccc4)c3)cc2)cc1 ; c1ccc(-c2ccc(N(c3cccc(-c4ccc(-c5ccccc5)c5oc6c(-c7ccccc7)cccc6c45)c3)c3cc(-c4ccccc4)ccc3-c3ccccc3)cc2)cc1]

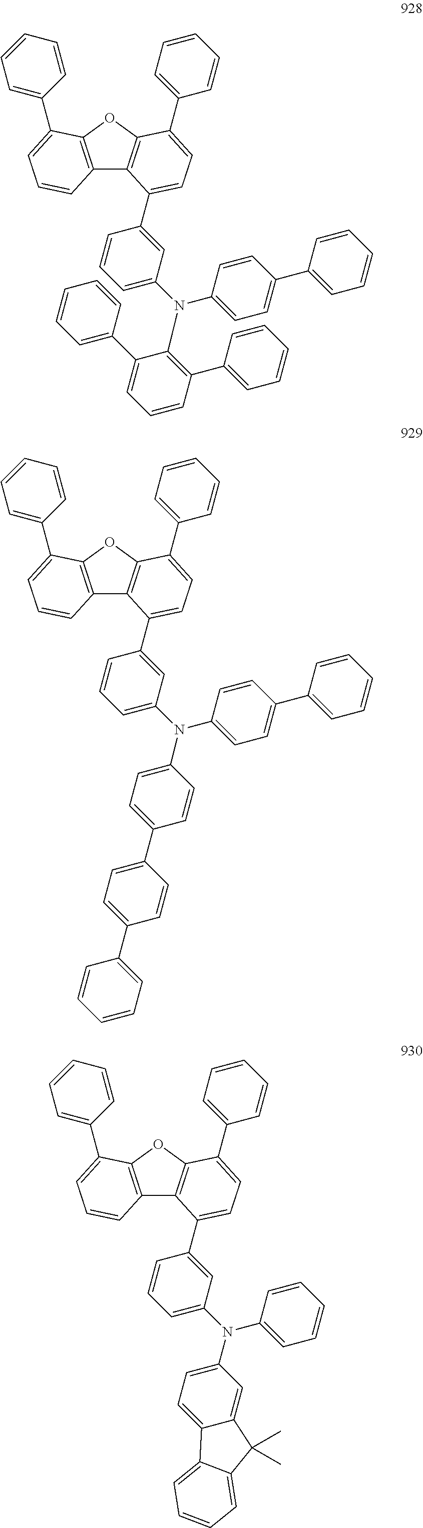

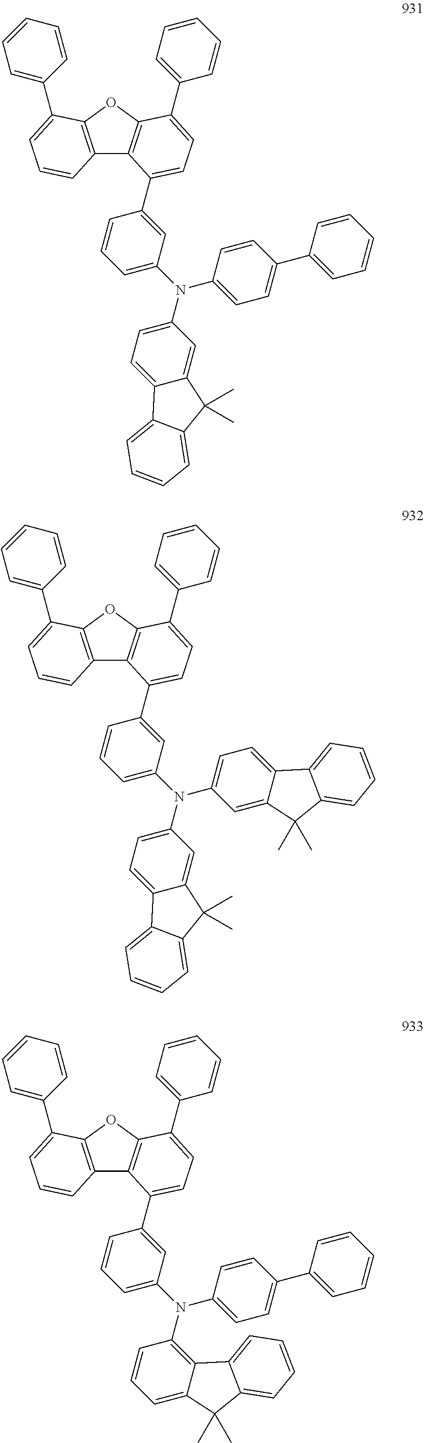 [SMILES: CC1(C)c2ccccc2-c2ccc(N(c3ccc(-c4ccccc4)cc3)c3cccc(-c4ccc(-c5ccccc5)c5oc6c(-c7ccccc7)cccc6c45)c3)cc21 ; CC1(C)c2ccccc2-c2ccc(N(c3cccc(-c4ccc(-c5ccccc5)c5oc6c(-c7ccccc7)cccc6c45)c3)c3ccc4c(c3)C(C)(C)c3ccccc3-4)cc21 ; CC1(C)c2ccccc2-c2c(N(c3ccc(-c4ccccc4)cc3)c3cccc(-c4ccc(-c5ccccc5)c5oc6c(-c7ccccc7)cccc6c45)c3)cccc21]

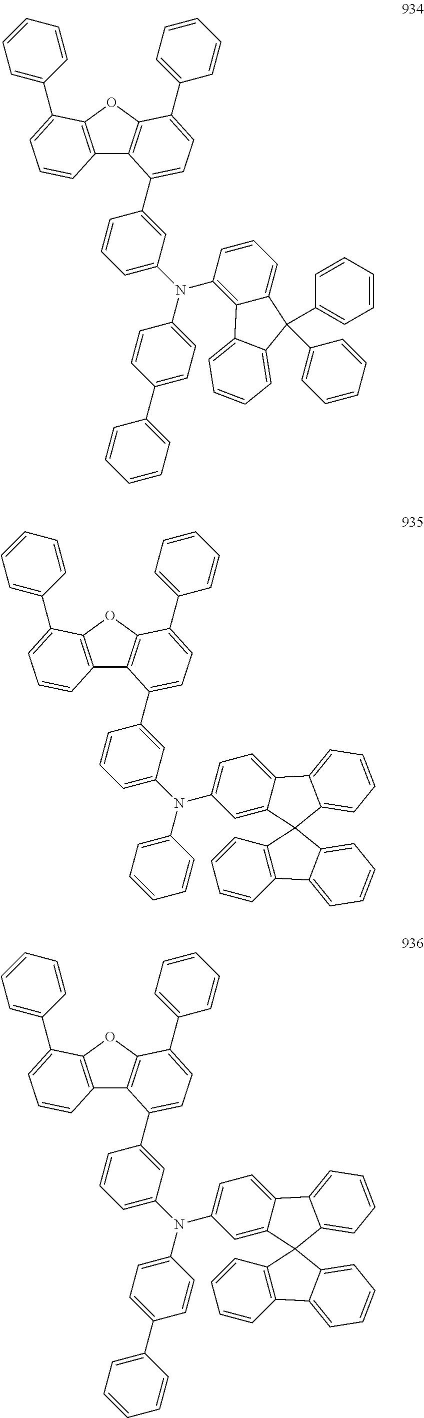 [SMILES: c1ccc(-c2ccc(N(c3cccc(-c4ccc(-c5ccccc5)c5oc6c(-c7ccccc7)cccc6c45)c3)c3cccc4c3-c3ccccc3C4(c3ccccc3)c3ccccc3)cc2)cc1 ; c1ccc(-c2cccc3c2oc2c(-c4ccccc4)ccc(-c4cccc(N(c5ccccc5)c5ccc6c(c5)C5(c7ccccc7-c7ccccc75)c5ccccc5-6)c4)c23)cc1 ; c1ccc(-c2ccc(N(c3cccc(-c4ccc(-c5ccccc5)c5oc6c(-c7ccccc7)cccc6c45)c3)c3ccc4c(c3)C3(c5ccccc5-c5ccccc53)c3ccccc3-4)cc2)cc1]

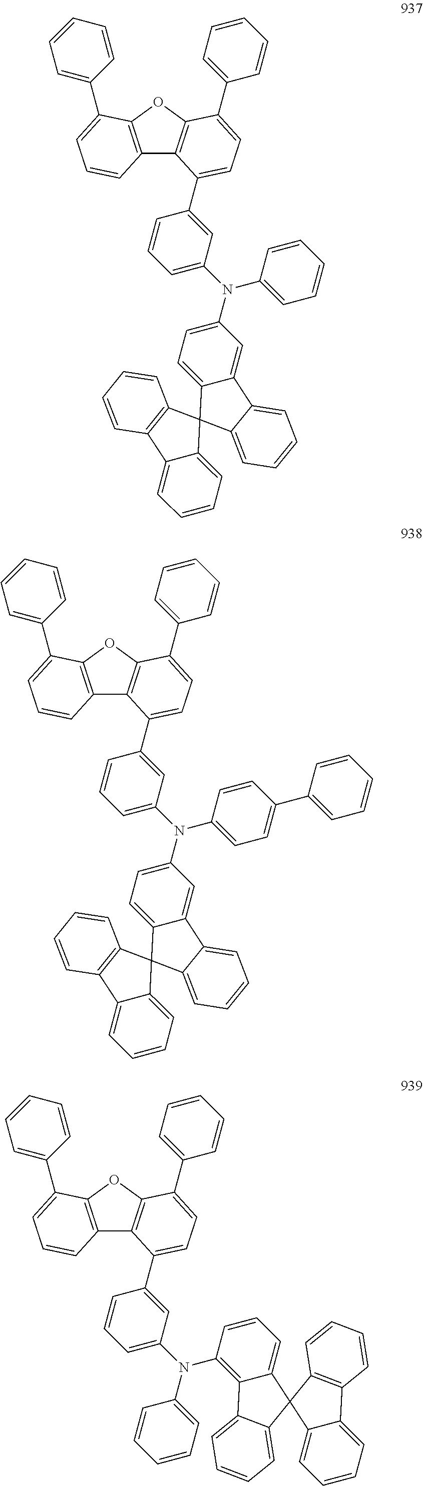

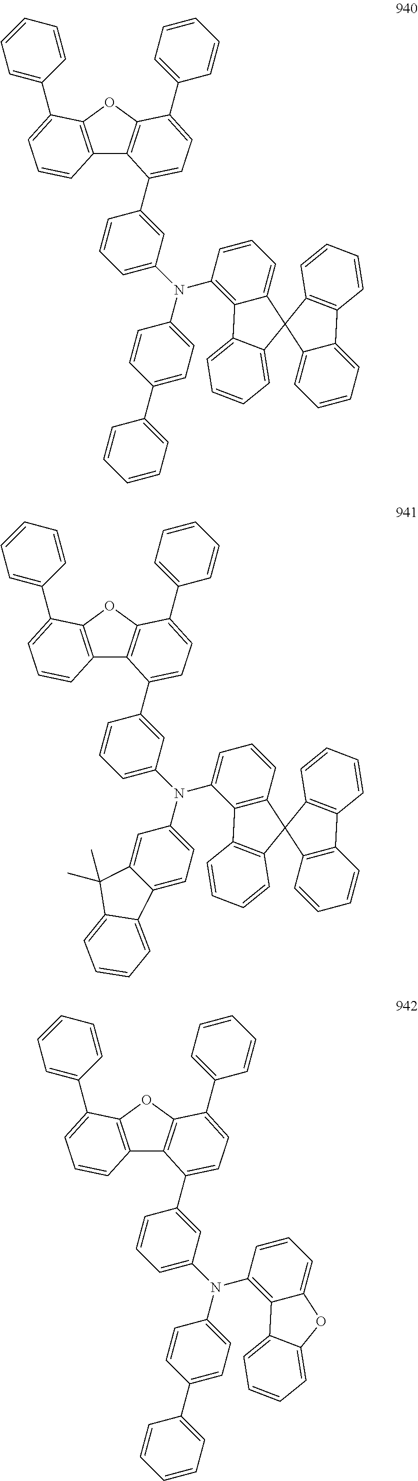 [SMILES: c1ccc(-c2ccc(N(c3cccc(-c4ccc(-c5ccccc5)c5oc6c(-c7ccccc7)cccc6c45)c3)c3cccc4c3-c3ccccc3C43c4ccccc4-c4ccccc43)cc2)cc1 ; CC1(C)c2ccccc2-c2ccc(N(c3cccc(-c4ccc(-c5ccccc5)c5oc6c(-c7ccccc7)cccc6c45)c3)c3cccc4c3-c3ccccc3C43c4ccccc4-c4ccccc43)cc21 ; c1ccc(-c2ccc(N(c3cccc(-c4ccc(-c5ccccc5)c5oc6c(-c7ccccc7)cccc6c45)c3)c3cccc4oc5ccccc5c34)cc2)cc1]

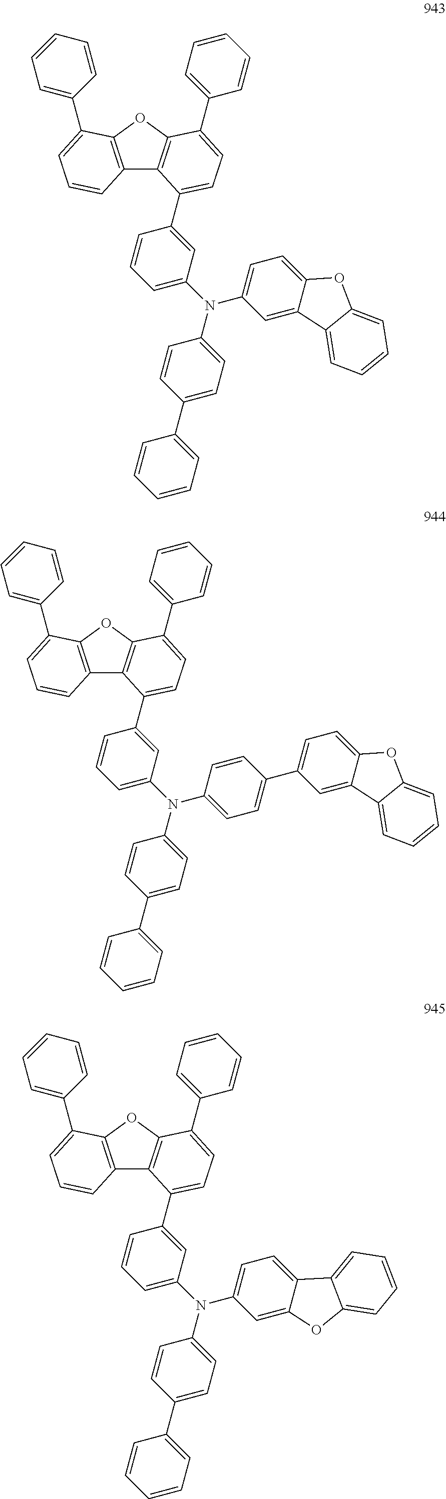 [SMILES: c1ccc(-c2ccc(N(c3cccc(-c4ccc(-c5ccccc5)c5oc6c(-c7ccccc7)cccc6c45)c3)c3ccc4oc5ccccc5c4c3)cc2)cc1 ; c1ccc(-c2ccc(N(c3ccc(-c4ccc5oc6ccccc6c5c4)cc3)c3cccc(-c4ccc(-c5ccccc5)c5oc6c(-c7ccccc7)cccc6c45)c3)cc2)cc1 ; c1ccc(-c2ccc(N(c3cccc(-c4ccc(-c5ccccc5)c5oc6c(-c7ccccc7)cccc6c45)c3)c3ccc4c(c3)oc3ccccc34)cc2)cc1]

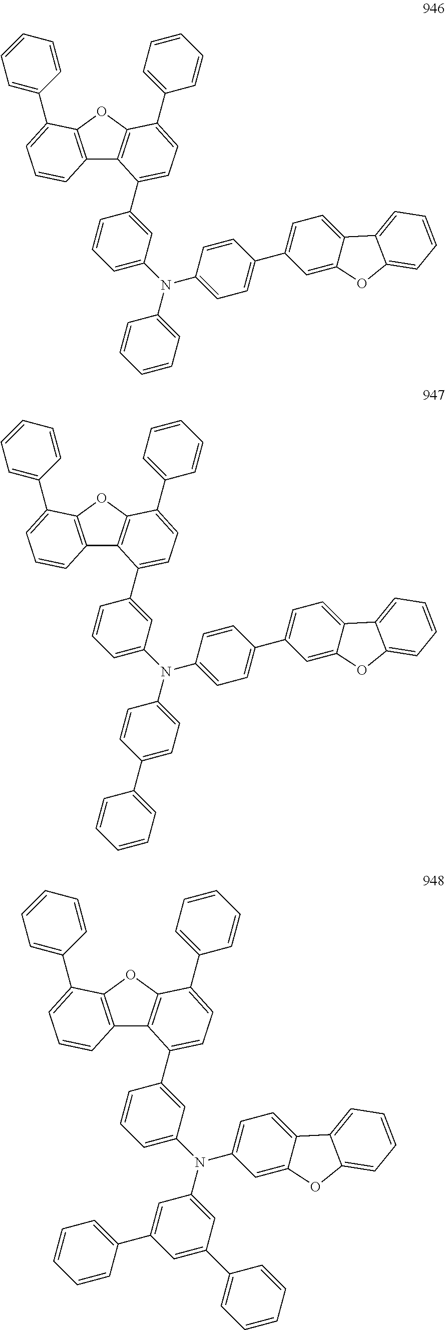 [SMILES: c1ccc(-c2cccc3c2oc2c(-c4ccccc4)ccc(-c4cccc(N(c5ccccc5)c5ccc(-c6ccc7c(c6)oc6ccccc67)cc5)c4)c23)cc1 ; c1ccc(-c2ccc(N(c3ccc(-c4ccc5c(c4)oc4ccccc45)cc3)c3cccc(-c4ccc(-c5ccccc5)c5oc6c(-c7ccccc7)cccc6c45)c3)cc2)cc1 ; c1ccc(-c2cc(-c3ccccc3)cc(N(c3cccc(-c4ccc(-c5ccccc5)c5oc6c(-c7ccccc7)cccc6c45)c3)c3ccc4c(c3)oc3ccccc34)c2)cc1]

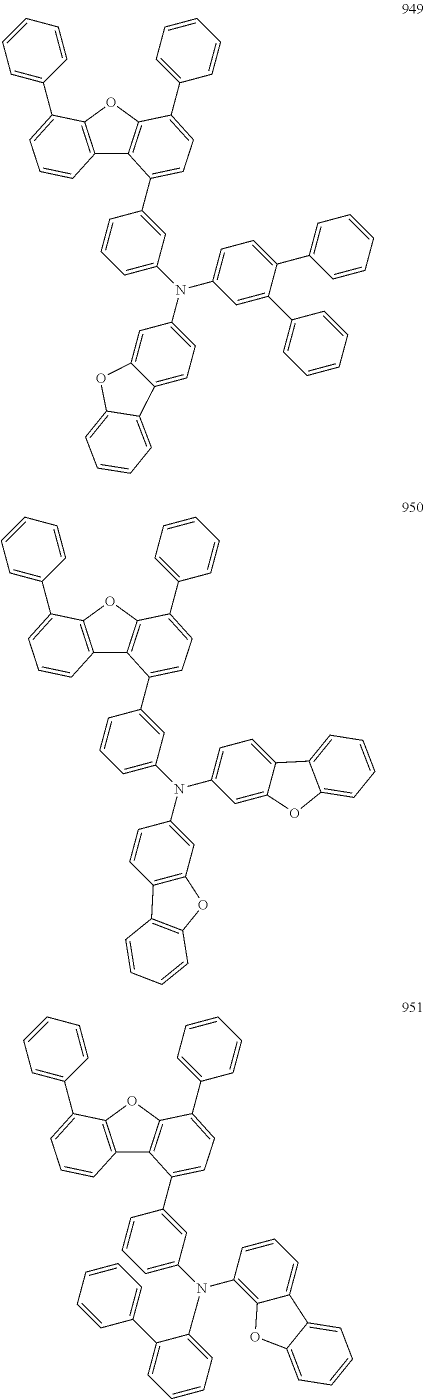 [SMILES: c1ccc(-c2ccc(N(c3cccc(-c4ccc(-c5ccccc5)c5oc6c(-c7ccccc7)cccc6c45)c3)c3ccc4c(c3)oc3ccccc34)cc2-c2ccccc2)cc1 ; c1ccc(-c2cccc3c2oc2c(-c4ccccc4)ccc(-c4cccc(N(c5ccc6c(c5)oc5ccccc56)c5ccc6c(c5)oc5ccccc56)c4)c23)cc1 ; c1ccc(-c2ccccc2N(c2cccc(-c3ccc(-c4ccccc4)c4oc5c(-c6ccccc6)cccc5c34)c2)c2cccc3c2oc2ccccc23)cc1]

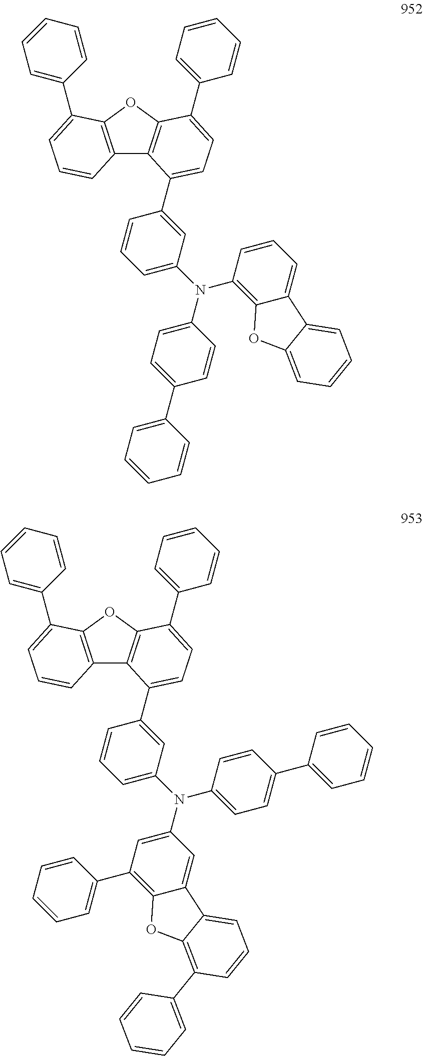 [SMILES: c1ccc(-c2ccc(N(c3cccc(-c4ccc(-c5ccccc5)c5oc6c(-c7ccccc7)cccc6c45)c3)c3cccc4c3oc3ccccc34)cc2)cc1 ; c1ccc(-c2ccc(N(c3cccc(-c4ccc(-c5ccccc5)c5oc6c(-c7ccccc7)cccc6c45)c3)c3cc(-c4ccccc4)c4oc5c(-c6ccccc6)cccc5c4c3)cc2)cc1]

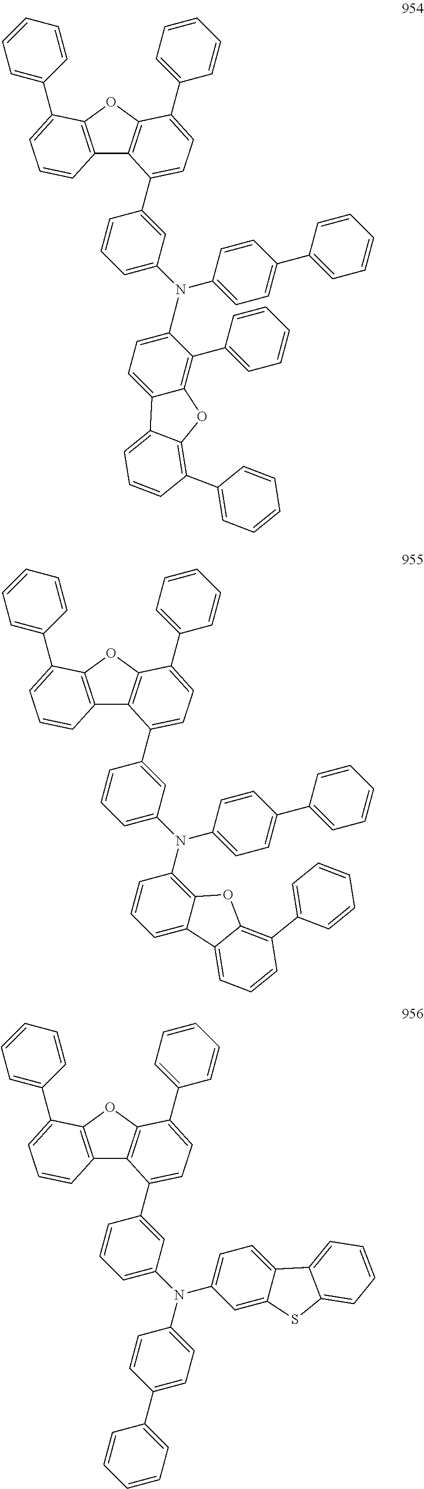 [SMILES: c1ccc(-c2ccc(N(c3cccc(-c4ccc(-c5ccccc5)c5oc6c(-c7ccccc7)cccc6c45)c3)c3ccc4c(oc5c(-c6ccccc6)cccc54)c3-c3ccccc3)cc2)cc1 ; c1ccc(-c2ccc(N(c3cccc(-c4ccc(-c5ccccc5)c5oc6c(-c7ccccc7)cccc6c45)c3)c3cccc4c3oc3c(-c5ccccc5)cccc34)cc2)cc1 ; c1ccc(-c2ccc(N(c3cccc(-c4ccc(-c5ccccc5)c5oc6c(-c7ccccc7)cccc6c45)c3)c3ccc4c(c3)sc3ccccc34)cc2)cc1]

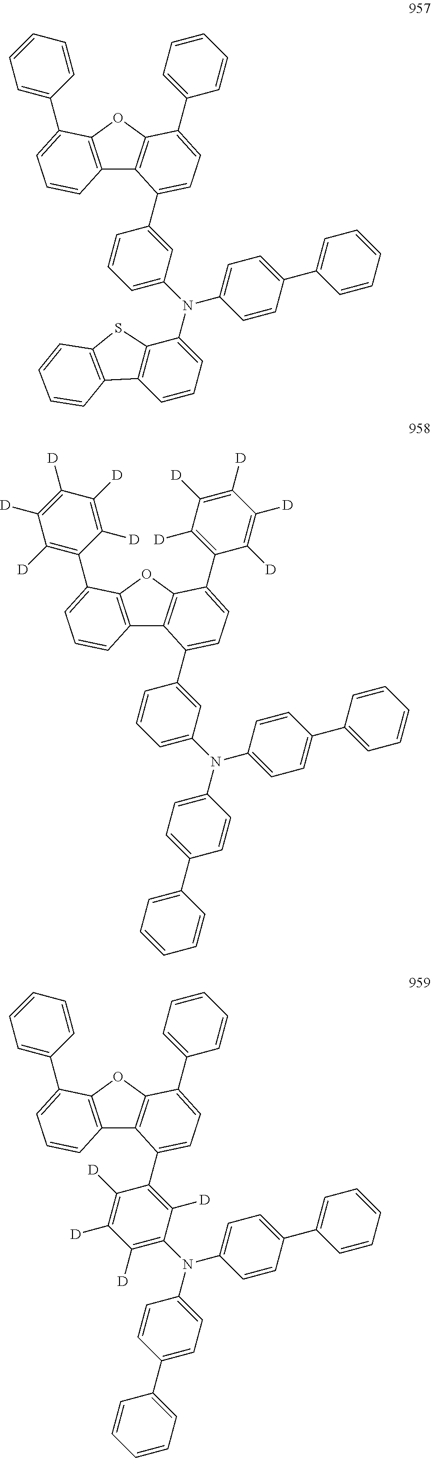 [SMILES: c1ccc(-c2ccc(N(c3cccc(-c4ccc(-c5ccccc5)c5oc6c(-c7ccccc7)cccc6c45)c3)c3cccc4c3sc3ccccc34)cc2)cc1 ; [2H]c1c([2H])c([2H])c(-c2cccc3c2oc2c(-c4c([2H])c([2H])c([2H])c([2H])c4[2H])ccc(-c4cccc(N(c5ccc(-c6ccccc6)cc5)c5ccc(-c6ccccc6)cc5)c4)c23)c([2H])c1[2H] ; [2H]c1c([2H])c(-c2ccc(-c3ccccc3)c3oc4c(-c5ccccc5)cccc4c23)c([2H])c(N(c2ccc(-c3ccccc3)cc2)c2ccc(-c3ccccc3)cc2)c1[2H]]

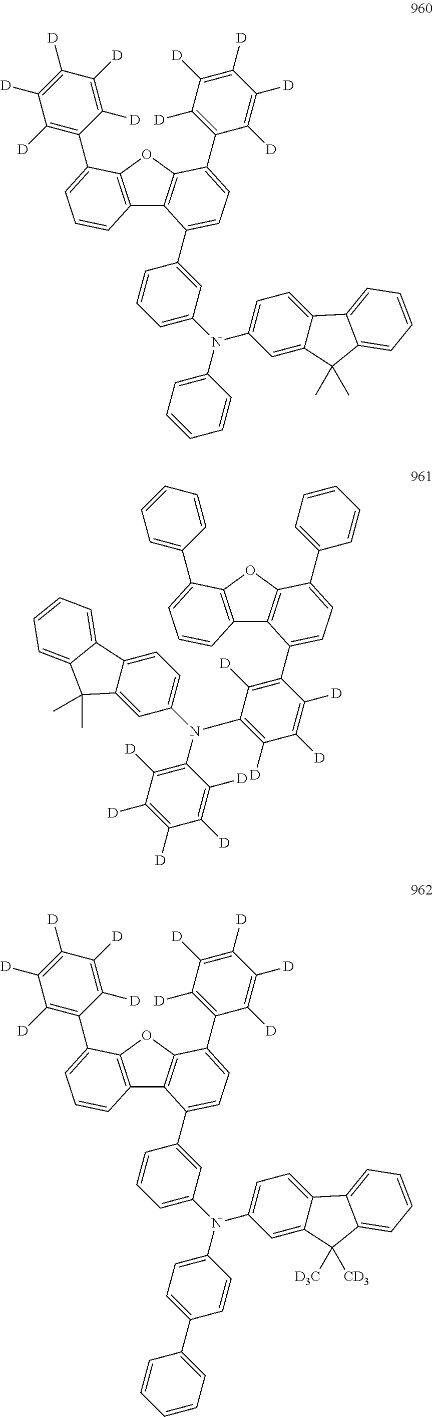 [SMILES: [2H]c1c([2H])c([2H])c(-c2cccc3c2oc2c(-c4c([2H])c([2H])c([2H])c([2H])c4[2H])ccc(-c4cccc(N(c5ccccc5)c5ccc6c(c5)C(C)(C)c5ccccc5-6)c4)c23)c([2H])c1[2H] ; [2H]c1c([2H])c([2H])c(N(c2ccc3c(c2)C(C)(C)c2ccccc2-3)c2c([2H])c([2H])c([2H])c(-c3ccc(-c4ccccc4)c4oc5c(-c6ccccc6)cccc5c34)c2[2H])c([2H])c1[2H] ; [2H]c1c([2H])c([2H])c(-c2cccc3c2oc2c(-c4c([2H])c([2H])c([2H])c([2H])c4[2H])ccc(-c4cccc(N(c5ccc(-c6ccccc6)cc5)c5ccc6c(c5)C(C([2H])([2H])[2H])(C([2H])([2H])[2H])c5ccccc5-6)c4)c23)c([2H])c1[2H]]

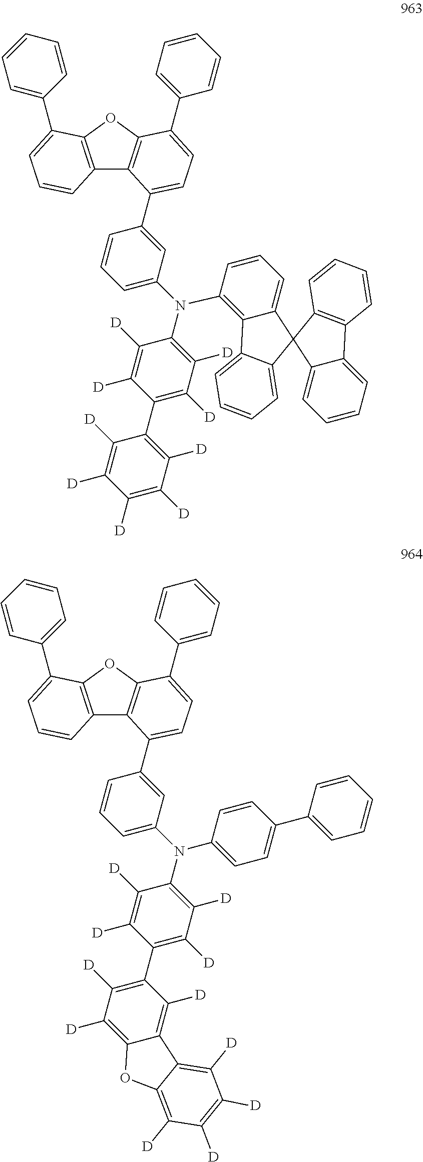 [SMILES: [2H]c1c([2H])c([2H])c(-c2c([2H])c([2H])c(N(c3cccc(-c4ccc(-c5ccccc5)c5oc6c(-c7ccccc7)cccc6c45)c3)c3cccc4c3-c3ccccc3C43c4ccccc4-c4ccccc43)c([2H])c2[2H])c([2H])c1[2H] ; [2H]c1c([2H])c(N(c2ccc(-c3ccccc3)cc2)c2cccc(-c3ccc(-c4ccccc4)c4oc5c(-c6ccccc6)cccc5c34)c2)c([2H])c([2H])c1-c1c([2H])c([2H])c2oc3c([2H])c([2H])c([2H])c([2H])c3c2c1[2H]]

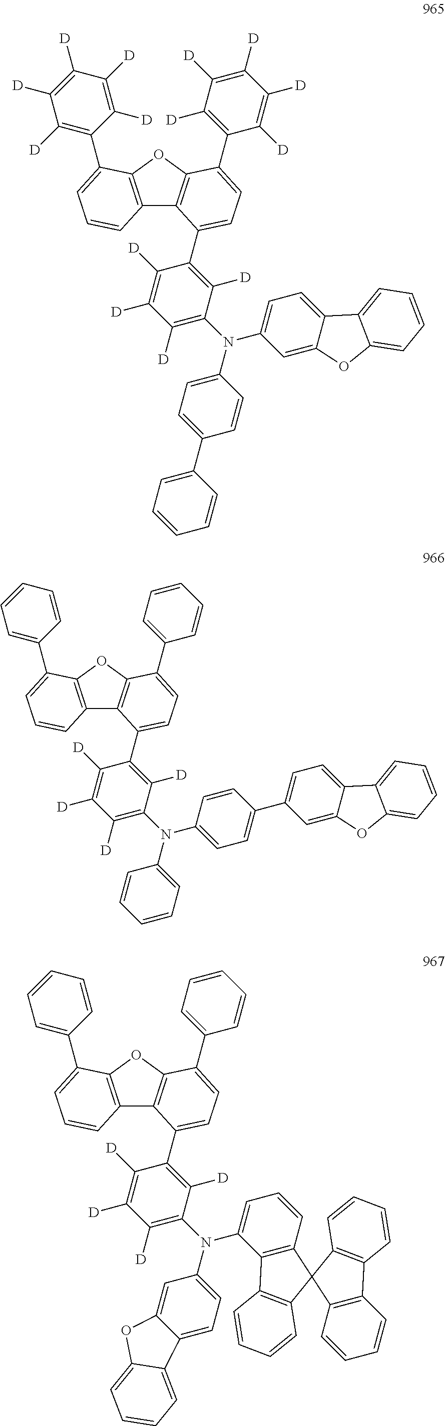 [SMILES: [2H]c1c([2H])c([2H])c(-c2cccc3c2oc2c(-c4c([2H])c([2H])c([2H])c([2H])c4[2H])ccc(-c4c([2H])c([2H])c([2H])c(N(c5ccc(-c6ccccc6)cc5)c5ccc6c(c5)oc5ccccc56)c4[2H])c23)c([2H])c1[2H] ; [2H]c1c([2H])c(-c2ccc(-c3ccccc3)c3oc4c(-c5ccccc5)cccc4c23)c([2H])c(N(c2ccccc2)c2ccc(-c3ccc4c(c3)oc3ccccc34)cc2)c1[2H] ; [2H]c1c([2H])c(-c2ccc(-c3ccccc3)c3oc4c(-c5ccccc5)cccc4c23)c([2H])c(N(c2ccc3c(c2)oc2ccccc23)c2cccc3c2-c2ccccc2C32c3ccccc3-c3ccccc32)c1[2H]]

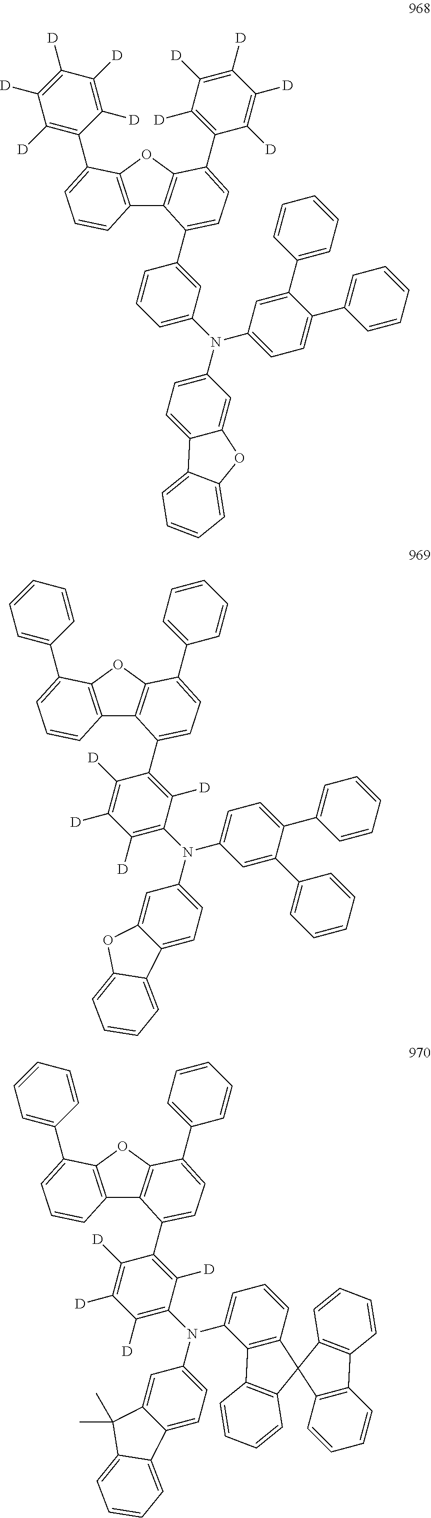

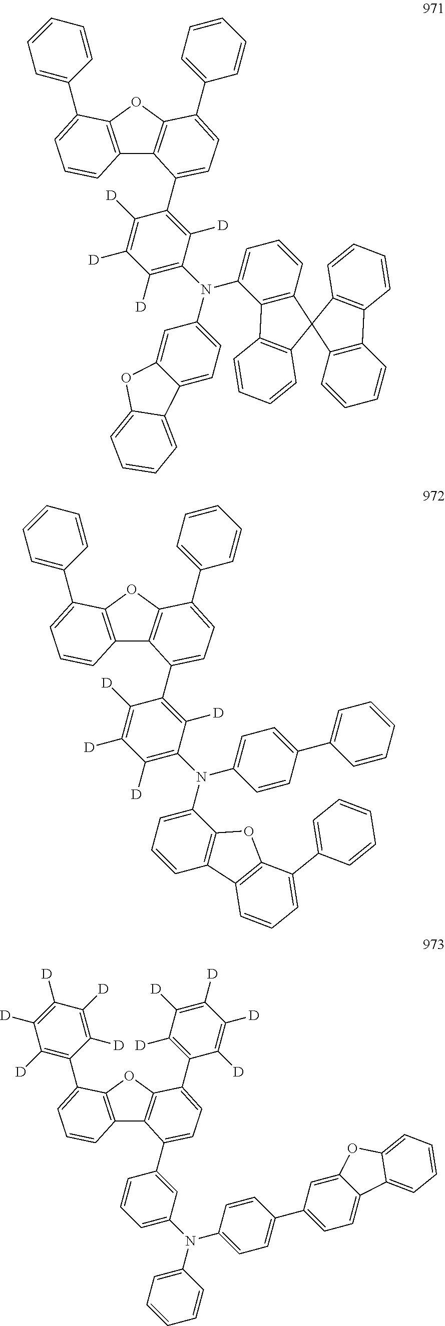 [SMILES: [2H]c1c([2H])c(-c2ccc(-c3ccccc3)c3oc4c(-c5ccccc5)cccc4c23)c([2H])c(N(c2ccc3c(c2)oc2ccccc23)c2cccc3c2-c2ccccc2C32c3ccccc3-c3ccccc32)c1[2H] ; [2H]c1c([2H])c(-c2ccc(-c3ccccc3)c3oc4c(-c5ccccc5)cccc4c23)c([2H])c(N(c2ccc(-c3ccccc3)cc2)c2cccc3c2oc2c(-c4ccccc4)cccc23)c1[2H] ; [2H]c1c([2H])c([2H])c(-c2cccc3c2oc2c(-c4c([2H])c([2H])c([2H])c([2H])c4[2H])ccc(-c4cccc(N(c5ccccc5)c5ccc(-c6ccc7c(c6)oc6ccccc67)cc5)c4)c23)c([2H])c1[2H]]

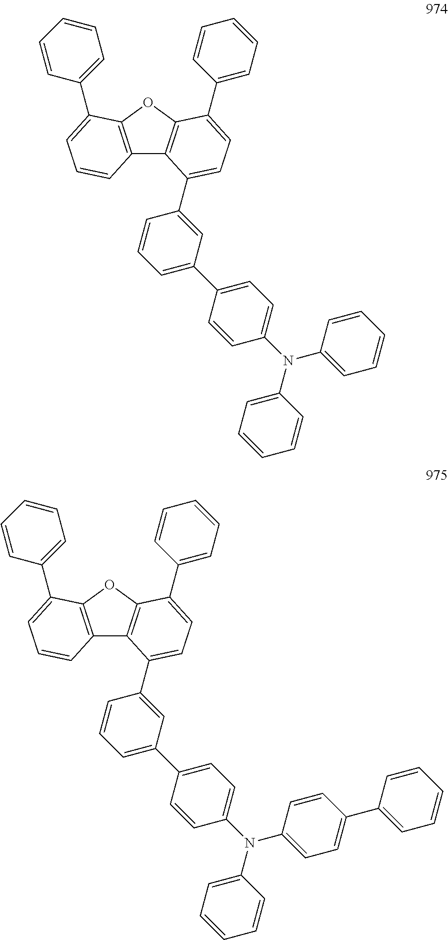 [SMILES: c1ccc(-c2cccc3c2oc2c(-c4ccccc4)ccc(-c4cccc(-c5ccc(N(c6ccccc6)c6ccccc6)cc5)c4)c23)cc1 ; c1ccc(-c2ccc(N(c3ccccc3)c3ccc(-c4cccc(-c5ccc(-c6ccccc6)c6oc7c(-c8ccccc8)cccc7c56)c4)cc3)cc2)cc1]

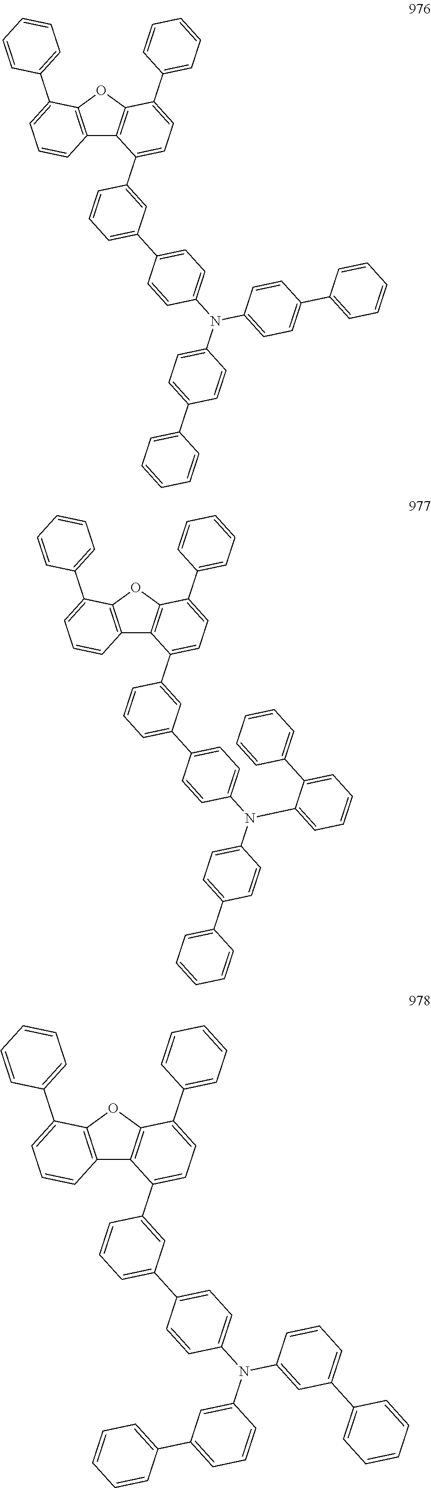

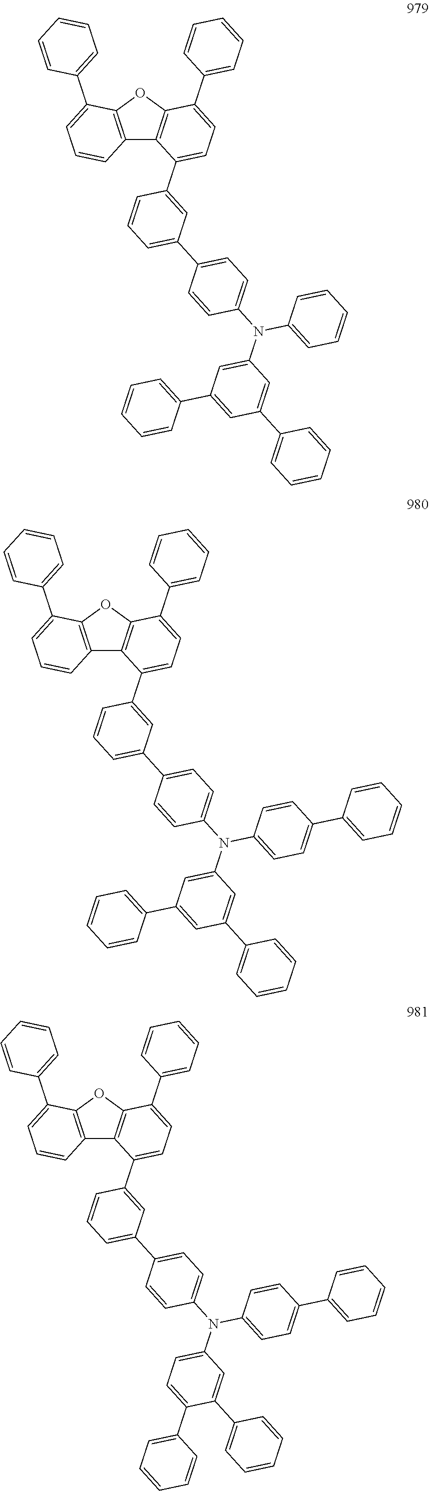 [SMILES: c1ccc(-c2cc(-c3ccccc3)cc(N(c3ccccc3)c3ccc(-c4cccc(-c5ccc(-c6ccccc6)c6oc7c(-c8ccccc8)cccc7c56)c4)cc3)c2)cc1 ; c1ccc(-c2ccc(N(c3ccc(-c4cccc(-c5ccc(-c6ccccc6)c6oc7c(-c8ccccc8)cccc7c56)c4)cc3)c3cc(-c4ccccc4)cc(-c4ccccc4)c3)cc2)cc1 ; c1ccc(-c2ccc(N(c3ccc(-c4cccc(-c5ccc(-c6ccccc6)c6oc7c(-c8ccccc8)cccc7c56)c4)cc3)c3ccc(-c4ccccc4)c(-c4ccccc4)c3)cc2)cc1]

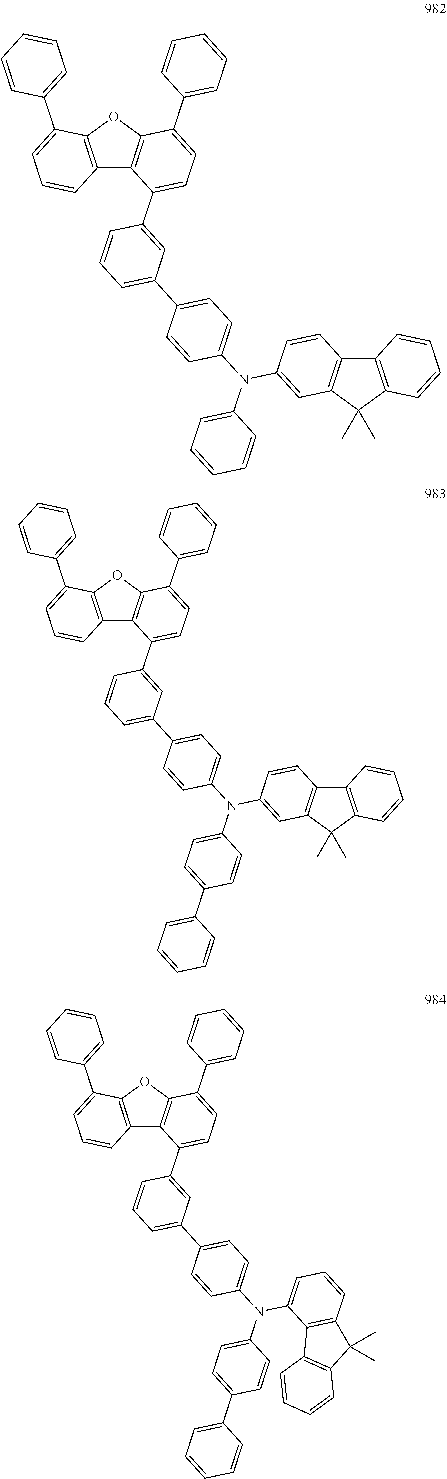

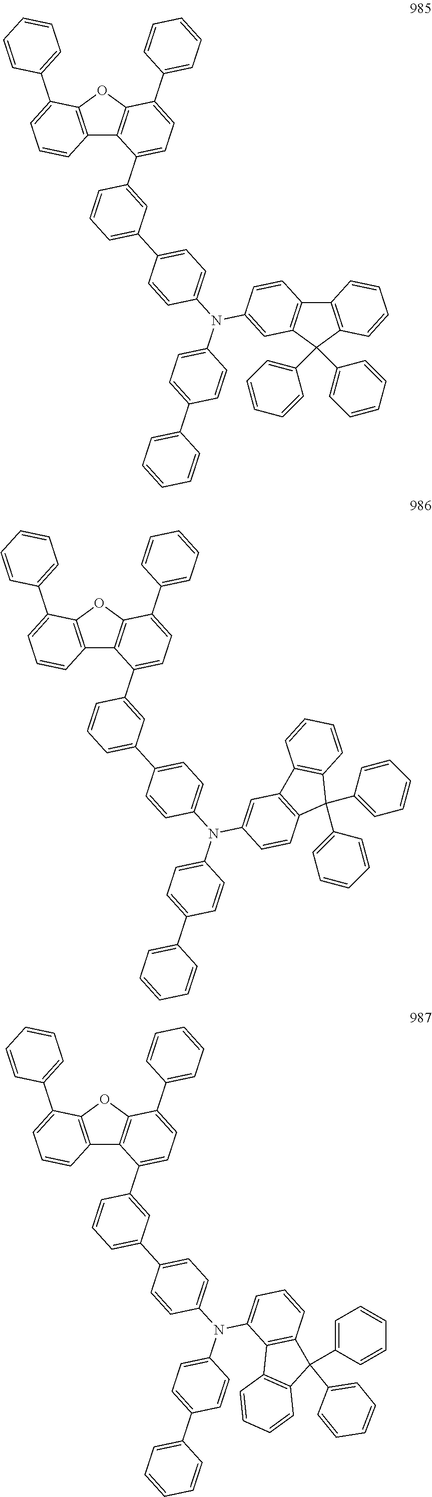

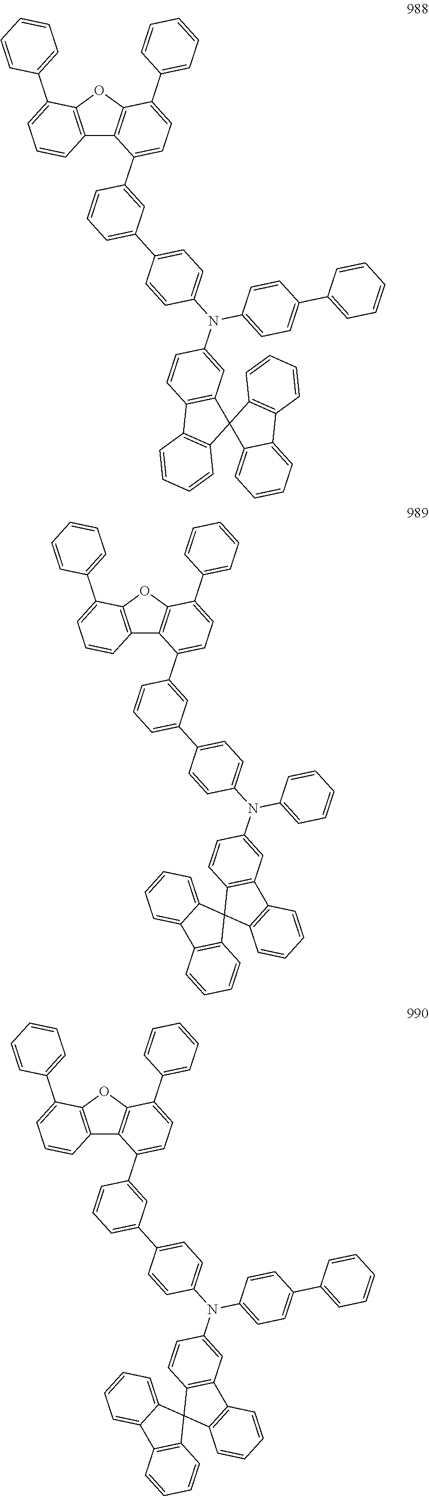

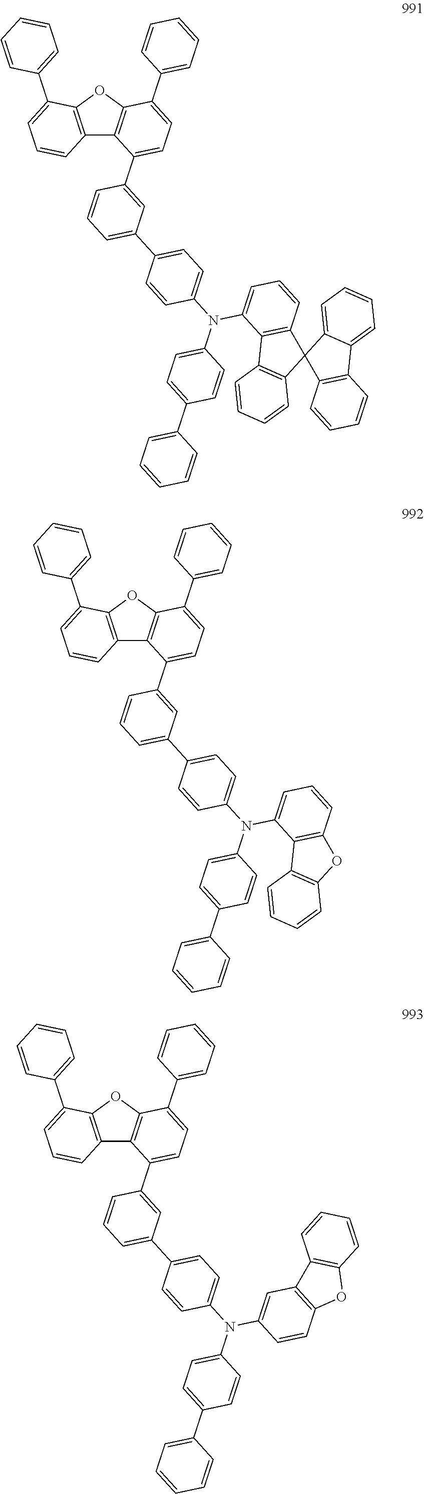

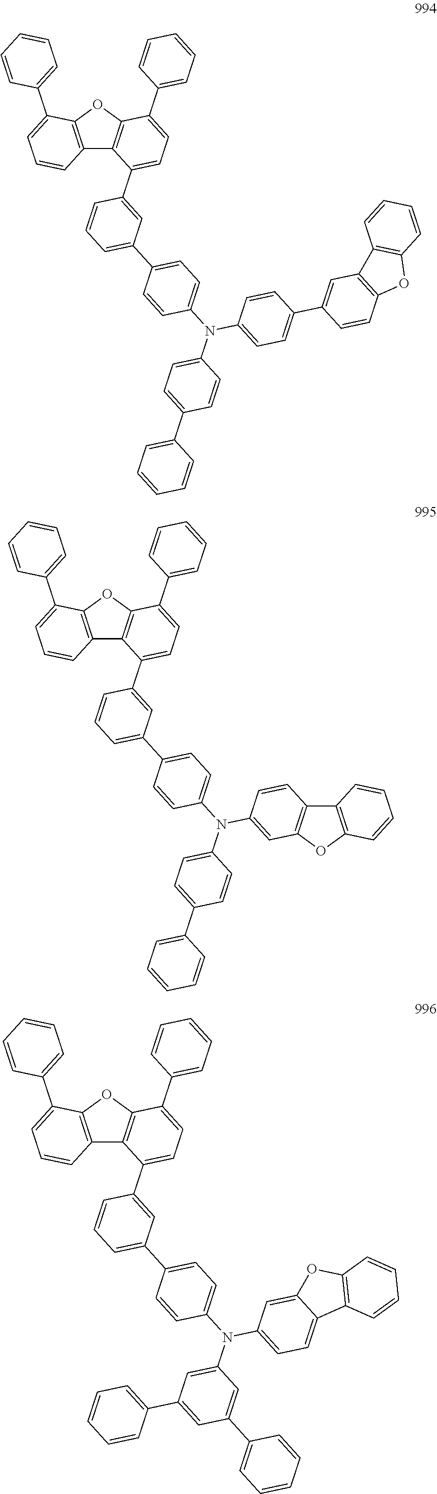

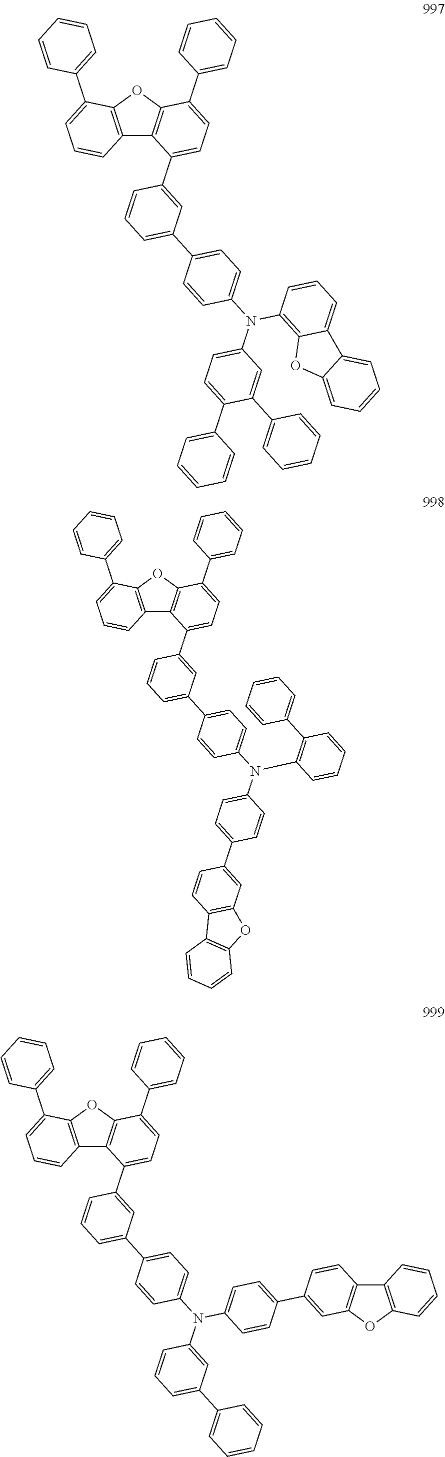

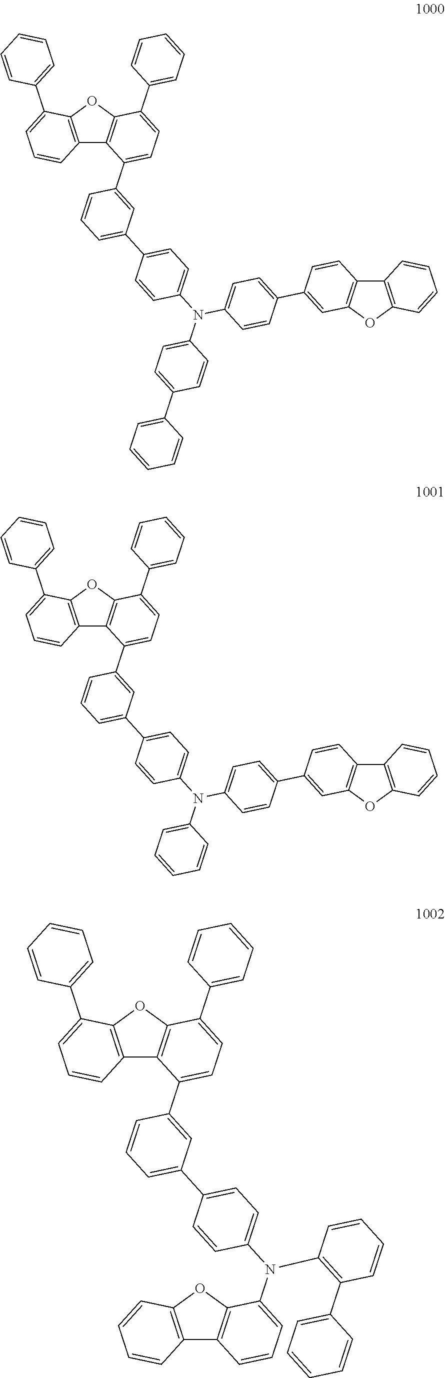 [SMILES: c1ccc(-c2ccc(N(c3ccc(-c4cccc(-c5ccc(-c6ccccc6)c6oc7c(-c8ccccc8)cccc7c56)c4)cc3)c3ccc(-c4ccc5c(c4)oc4ccccc45)cc3)cc2)cc1 ; c1ccc(-c2cccc3c2oc2c(-c4ccccc4)ccc(-c4cccc(-c5ccc(N(c6ccccc6)c6ccc(-c7ccc8c(c7)oc7ccccc78)cc6)cc5)c4)c23)cc1 ; c1ccc(-c2ccccc2N(c2ccc(-c3cccc(-c4ccc(-c5ccccc5)c5oc6c(-c7ccccc7)cccc6c45)c3)cc2)c2cccc3c2oc2ccccc23)cc1]

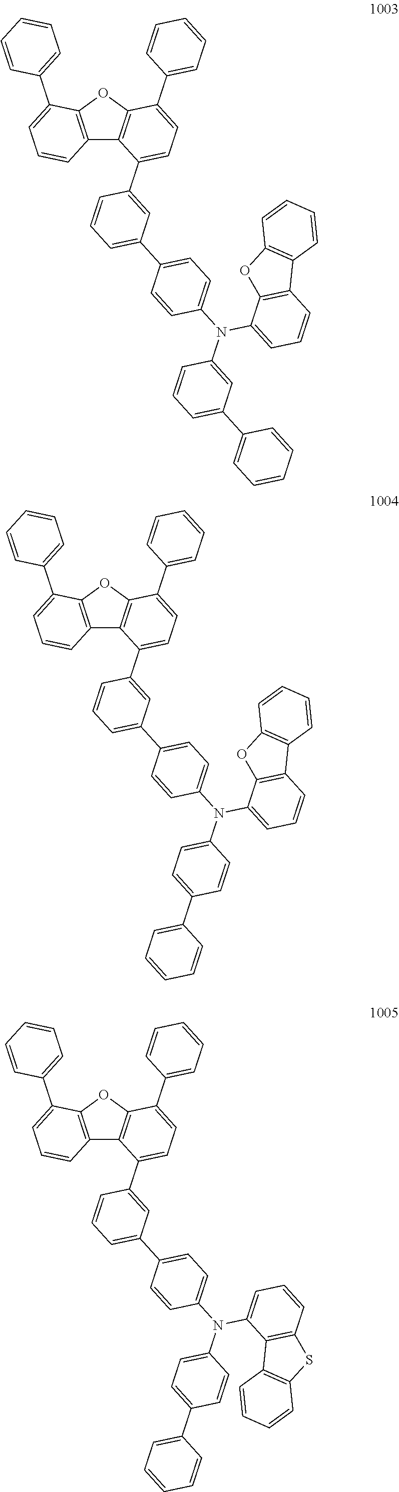 [SMILES: c1ccc(-c2cccc(N(c3ccc(-c4cccc(-c5ccc(-c6ccccc6)c6oc7c(-c8ccccc8)cccc7c56)c4)cc3)c3cccc4c3oc3ccccc34)c2)cc1 ; c1ccc(-c2ccc(N(c3ccc(-c4cccc(-c5ccc(-c6ccccc6)c6oc7c(-c8ccccc8)cccc7c56)c4)cc3)c3cccc4c3oc3ccccc34)cc2)cc1 ; c1ccc(-c2ccc(N(c3ccc(-c4cccc(-c5ccc(-c6ccccc6)c6oc7c(-c8ccccc8)cccc7c56)c4)cc3)c3cccc4sc5ccccc5c34)cc2)cc1]

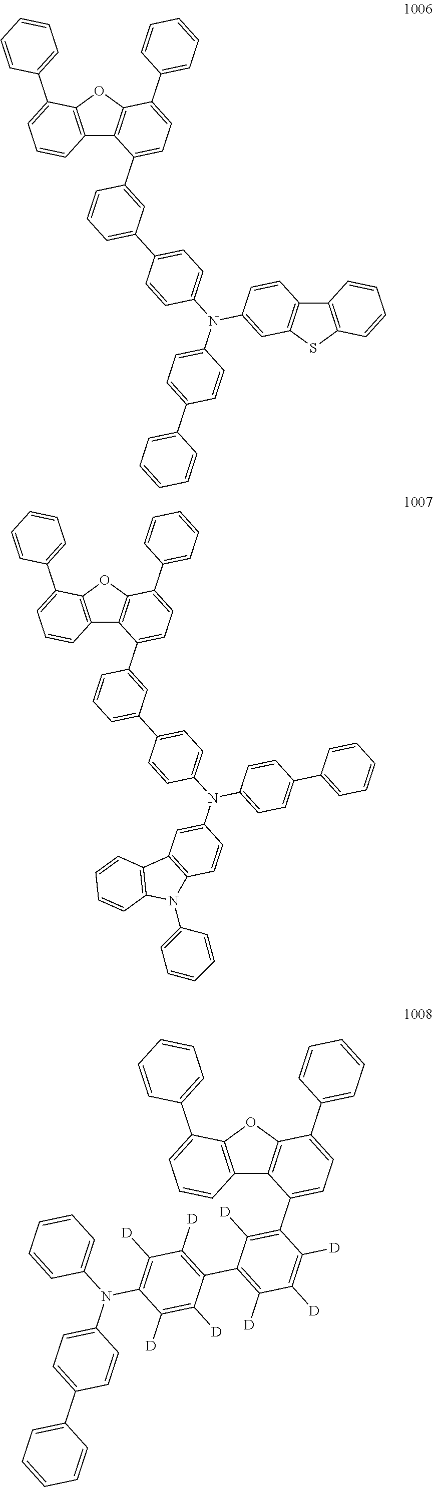

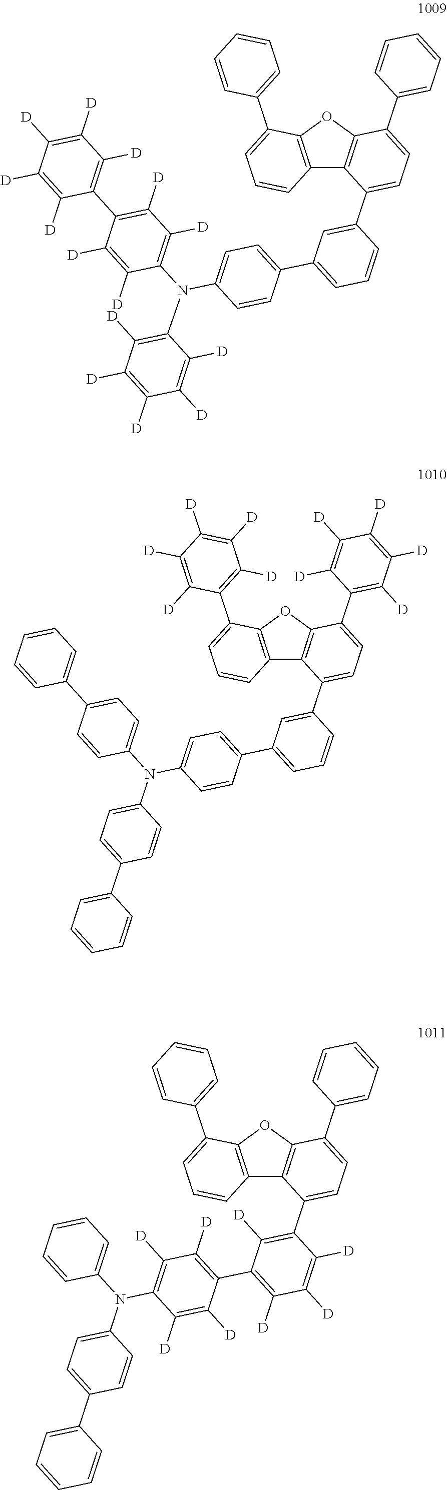 [SMILES: [2H]c1c([2H])c([2H])c(-c2c([2H])c([2H])c(N(c3ccc(-c4cccc(-c5ccc(-c6ccccc6)c6oc7c(-c8ccccc8)cccc7c56)c4)cc3)c3c([2H])c([2H])c([2H])c([2H])c3[2H])c([2H])c2[2H])c([2H])c1[2H] ; [2H]c1c([2H])c([2H])c(-c2cccc3c2oc2c(-c4c([2H])c([2H])c([2H])c([2H])c4[2H])ccc(-c4cccc(-c5ccc(N(c6ccc(-c7ccccc7)cc6)c6ccc(-c7ccccc7)cc6)cc5)c4)c23)c([2H])c1[2H] ; [2H]c1c([2H])c(-c2c([2H])c([2H])c(N(c3ccccc3)c3ccc(-c4ccccc4)cc3)c([2H])c2[2H])c([2H])c(-c2ccc(-c3ccccc3)c3oc4c(-c5ccccc5)cccc4c23)c1[2H]]

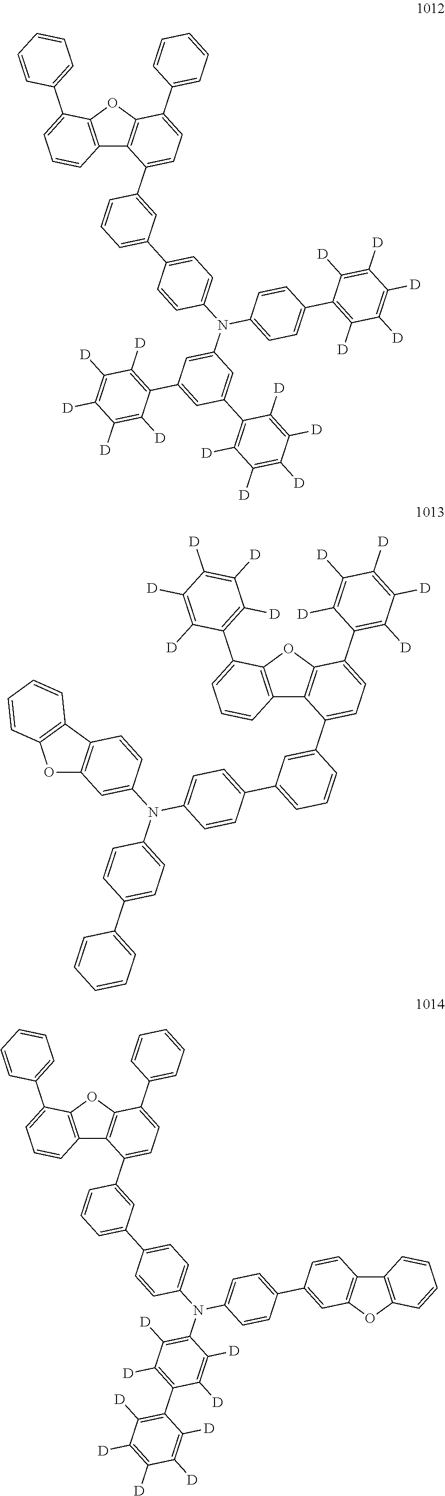

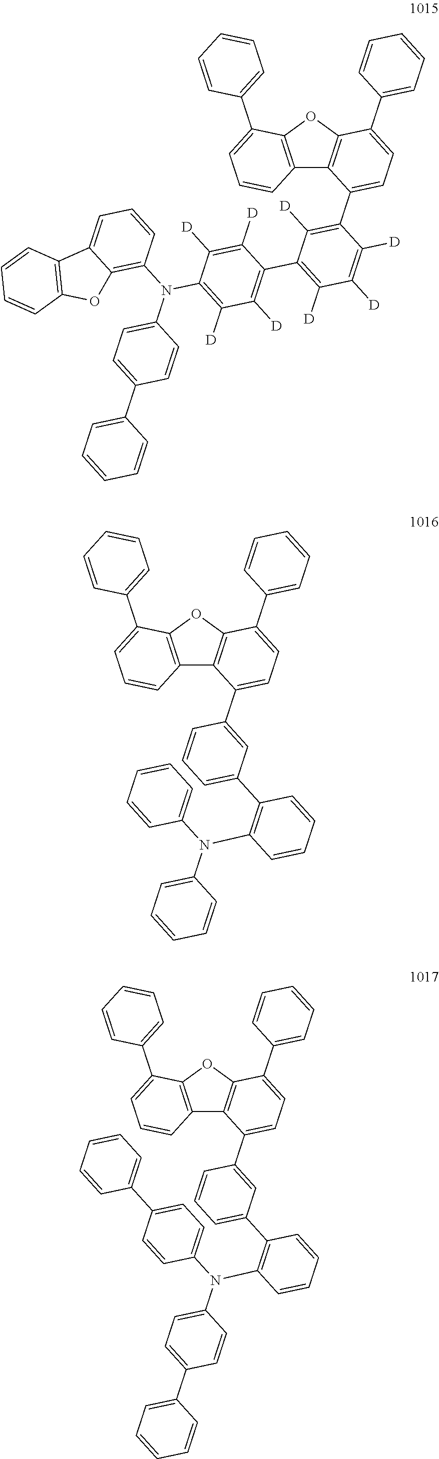 [SMILES: [2H]c1c([2H])c(-c2c([2H])c([2H])c(N(c3ccc(-c4ccccc4)cc3)c3cccc4c3oc3ccccc34)c([2H])c2[2H])c([2H])c(-c2ccc(-c3ccccc3)c3oc4c(-c5ccccc5)cccc4c23)c1[2H] ; c1ccc(-c2cccc3c2oc2c(-c4ccccc4)ccc(-c4cccc(-c5ccccc5N(c5ccccc5)c5ccccc5)c4)c23)cc1 ; c1ccc(-c2ccc(N(c3ccc(-c4ccccc4)cc3)c3ccccc3-c3cccc(-c4ccc(-c5ccccc5)c5oc6c(-c7ccccc7)cccc6c45)c3)cc2)cc1]

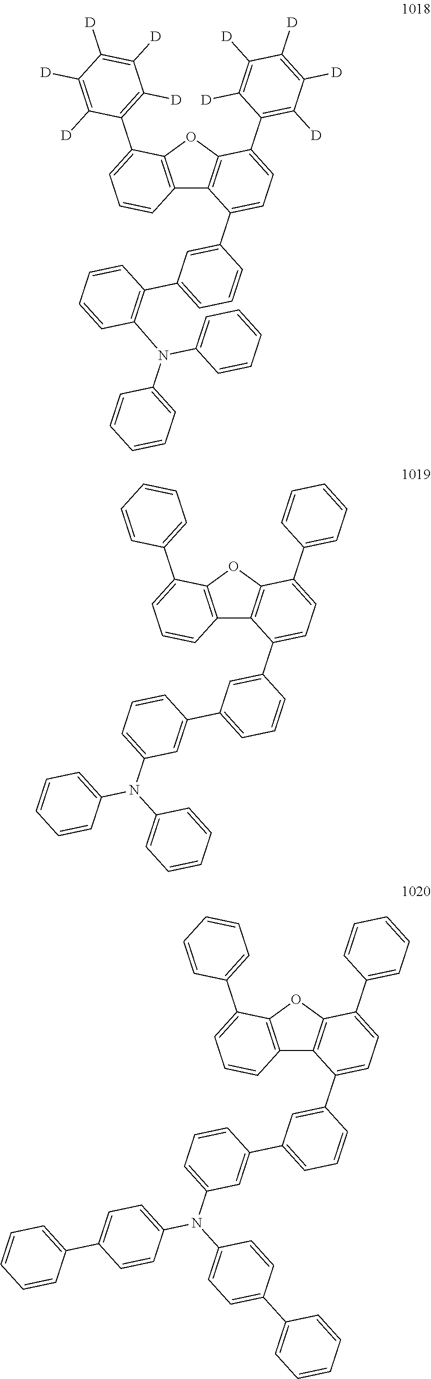

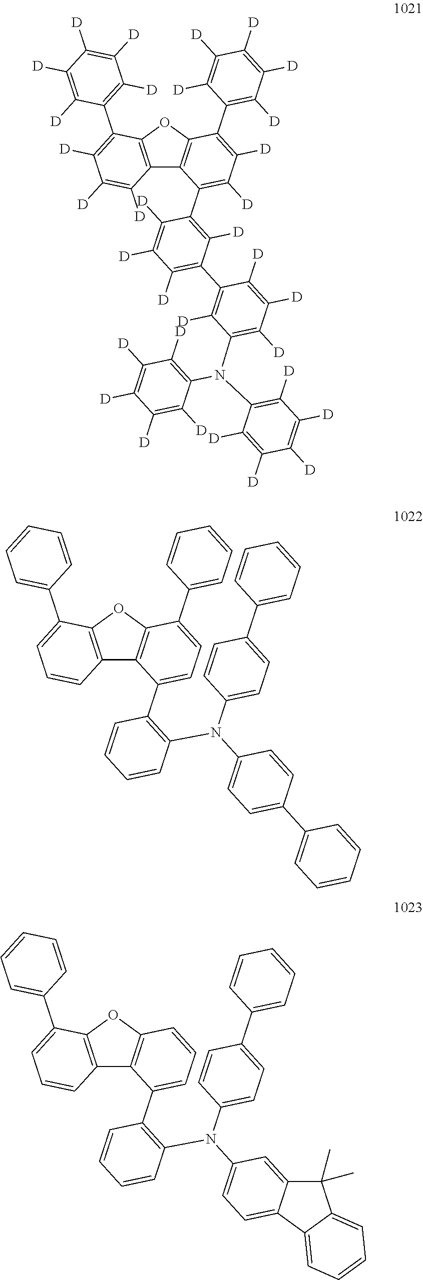 [SMILES: [2H]c1c([2H])c([2H])c(-c2c([2H])c([2H])c([2H])c3c2oc2c(-c4c([2H])c([2H])c([2H])c([2H])c4[2H])c([2H])c([2H])c(-c4c([2H])c([2H])c([2H])c(-c5c([2H])c([2H])c([2H])c(N(c6c([2H])c([2H])c([2H])c([2H])c6[2H])c6c([2H])c([2H])c([2H])c([2H])c6[2H])c5[2H])c4[2H])c23)c([2H])c1[2H] ; c1ccc(-c2ccc(N(c3ccc(-c4ccccc4)cc3)c3ccccc3-c3ccc(-c4ccccc4)c4oc5c(-c6ccccc6)cccc5c34)cc2)cc1 ; CC1(C)c2ccccc2-c2ccc(N(c3ccc(-c4ccccc4)cc3)c3ccccc3-c3cccc4oc5c(-c6ccccc6)cccc5c34)cc21]

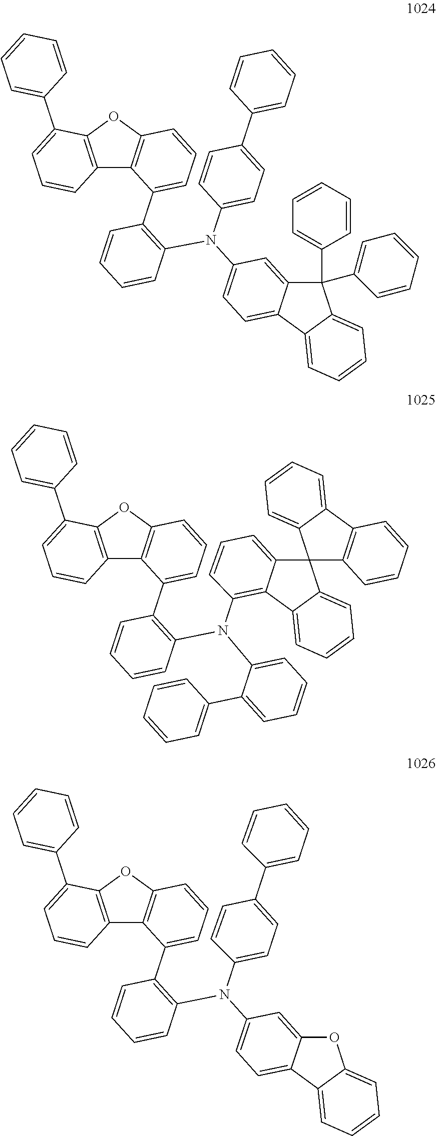 [SMILES: c1ccc(-c2ccc(N(c3ccc4c(c3)C(c3ccccc3)(c3ccccc3)c3ccccc3-4)c3ccccc3-c3cccc4oc5c(-c6ccccc6)cccc5c34)cc2)cc1 ; c1ccc(-c2ccccc2N(c2ccccc2-c2cccc3oc4c(-c5ccccc5)cccc4c23)c2cccc3c2-c2ccccc2C32c3ccccc3-c3ccccc32)cc1 ; c1ccc(-c2ccc(N(c3ccc4c(c3)oc3ccccc34)c3ccccc3-c3cccc4oc5c(-c6ccccc6)cccc5c34)cc2)cc1]

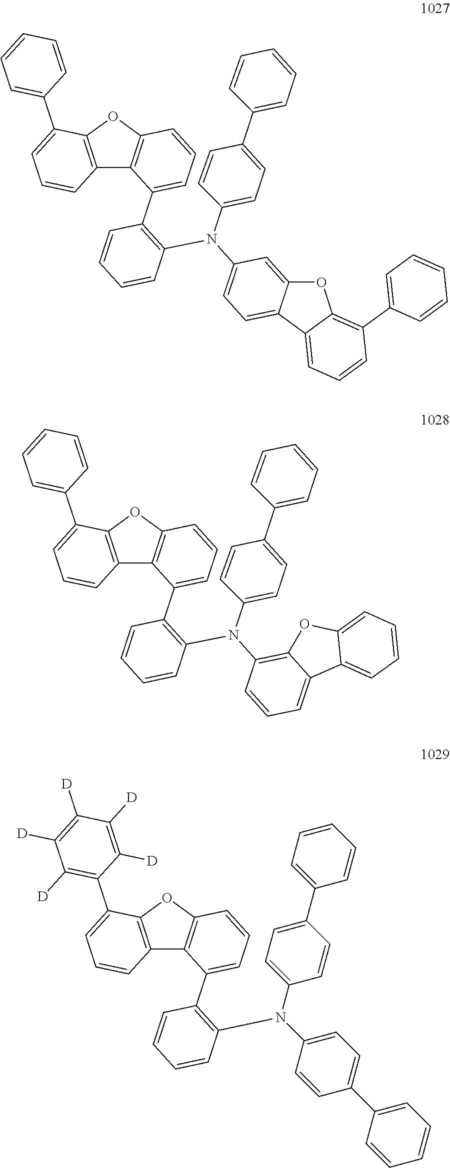 [SMILES: c1ccc(-c2ccc(N(c3ccc4c(c3)oc3c(-c5ccccc5)cccc34)c3ccccc3-c3cccc4oc5c(-c6ccccc6)cccc5c34)cc2)cc1 ; c1ccc(-c2ccc(N(c3ccccc3-c3cccc4oc5c(-c6ccccc6)cccc5c34)c3cccc4c3oc3ccccc34)cc2)cc1 ; [2H]c1c([2H])c([2H])c(-c2cccc3c2oc2cccc(-c4ccccc4N(c4ccc(-c5ccccc5)cc4)c4ccc(-c5ccccc5)cc4)c23)c([2H])c1[2H]]

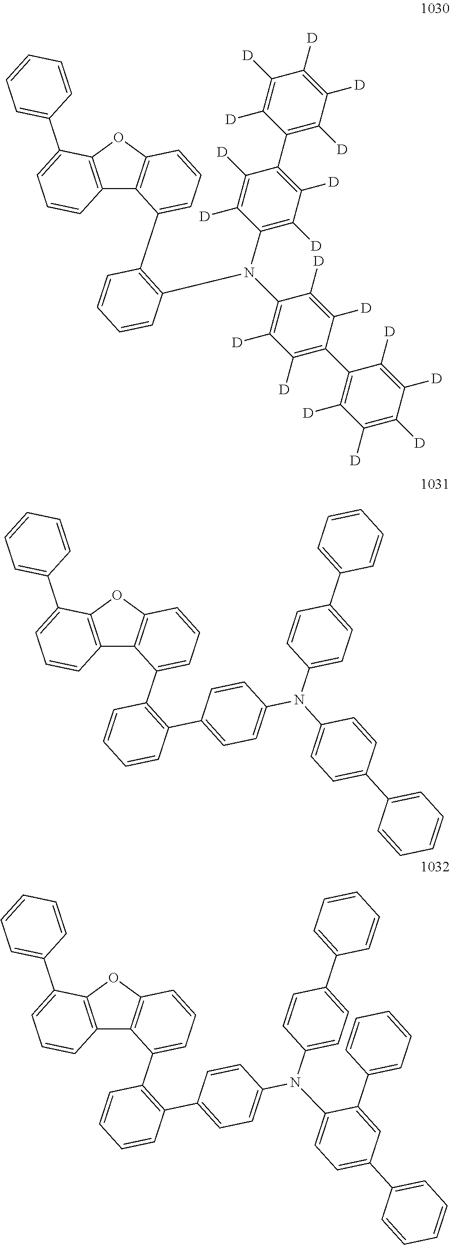 [SMILES: [2H]c1c([2H])c([2H])c(-c2c([2H])c([2H])c(N(c3ccccc3-c3cccc4oc5c(-c6ccccc6)cccc5c34)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3ccc(-c4ccccc4)cc3)c3ccc(-c4ccccc4-c4cccc5oc6c(-c7ccccc7)cccc6c45)cc3)cc2)cc1 ; c1ccc(-c2ccc(N(c3ccc(-c4ccccc4-c4cccc5oc6c(-c7ccccc7)cccc6c45)cc3)c3ccc(-c4ccccc4)cc3-c3ccccc3)cc2)cc1]

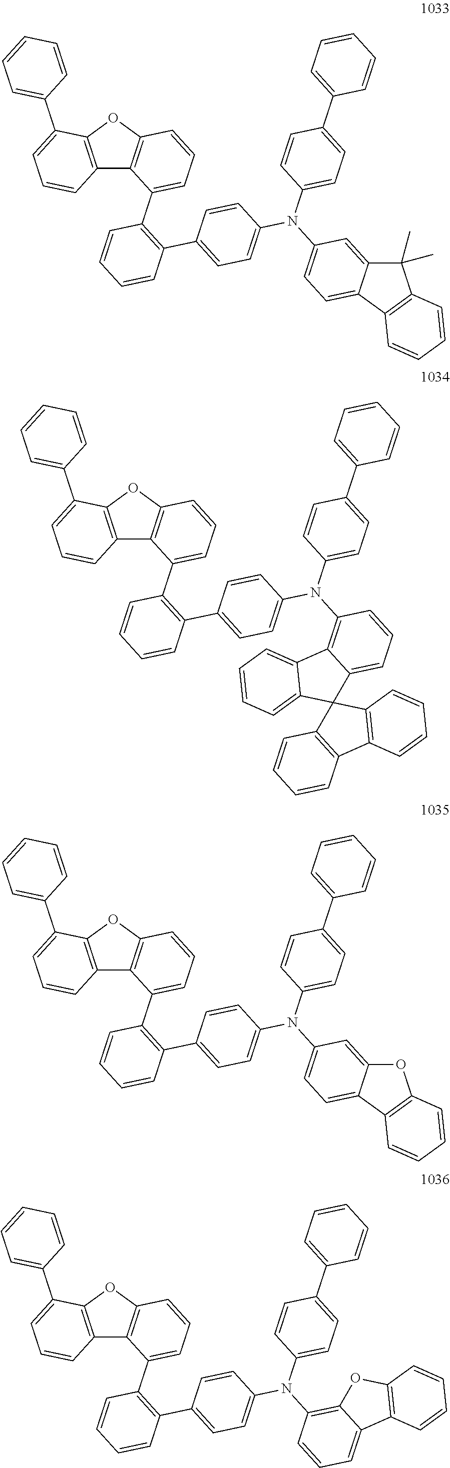 [SMILES: CC1(C)c2ccccc2-c2ccc(N(c3ccc(-c4ccccc4)cc3)c3ccc(-c4ccccc4-c4cccc5oc6c(-c7ccccc7)cccc6c45)cc3)cc21 ; c1ccc(-c2ccc(N(c3ccc(-c4ccccc4-c4cccc5oc6c(-c7ccccc7)cccc6c45)cc3)c3cccc4c3-c3ccccc3C43c4ccccc4-c4ccccc43)cc2)cc1 ; c1ccc(-c2ccc(N(c3ccc(-c4ccccc4-c4cccc5oc6c(-c7ccccc7)cccc6c45)cc3)c3ccc4c(c3)oc3ccccc34)cc2)cc1 ; c1ccc(-c2ccc(N(c3ccc(-c4ccccc4-c4cccc5oc6c(-c7ccccc7)cccc6c45)cc3)c3cccc4c3oc3ccccc34)cc2)cc1]

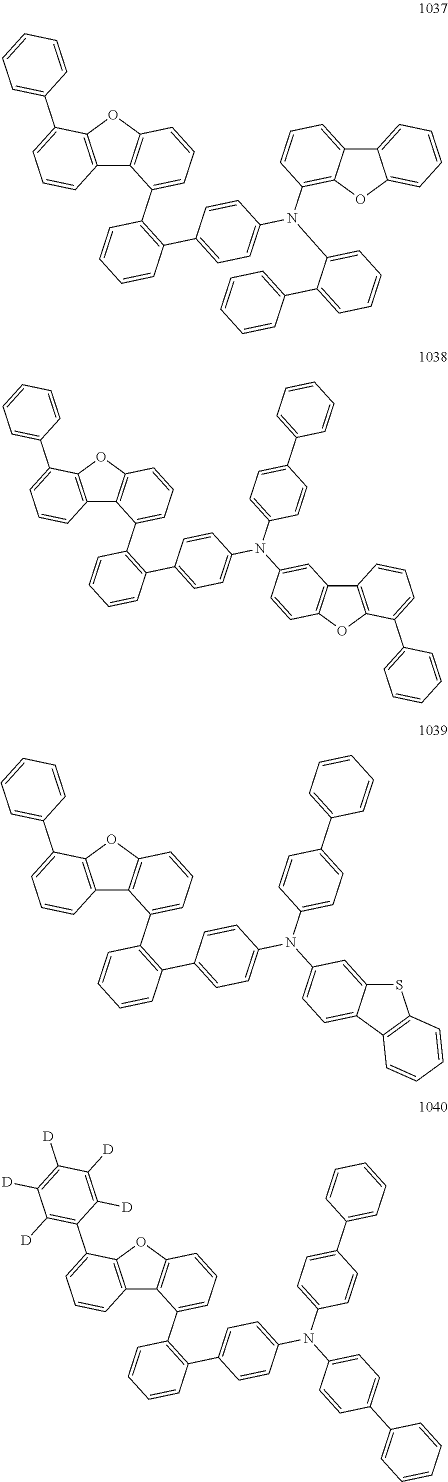 [SMILES: c1ccc(-c2ccccc2N(c2ccc(-c3ccccc3-c3cccc4oc5c(-c6ccccc6)cccc5c34)cc2)c2cccc3c2oc2ccccc23)cc1 ; c1ccc(-c2ccc(N(c3ccc(-c4ccccc4-c4cccc5oc6c(-c7ccccc7)cccc6c45)cc3)c3ccc4oc5c(-c6ccccc6)cccc5c4c3)cc2)cc1 ; c1ccc(-c2ccc(N(c3ccc(-c4ccccc4-c4cccc5oc6c(-c7ccccc7)cccc6c45)cc3)c3ccc4c(c3)sc3ccccc34)cc2)cc1 ; [2H]c1c([2H])c([2H])c(-c2cccc3c2oc2cccc(-c4ccccc4-c4ccc(N(c5ccc(-c6ccccc6)cc5)c5ccc(-c6ccccc6)cc5)cc4)c23)c([2H])c1[2H]]

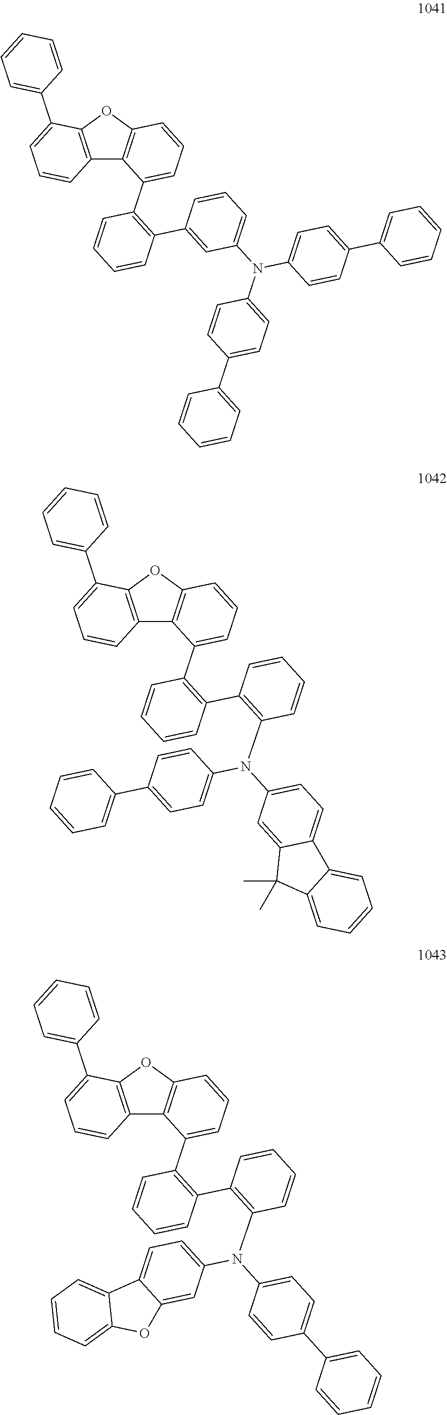 [SMILES: c1ccc(-c2ccc(N(c3ccc(-c4ccccc4)cc3)c3cccc(-c4ccccc4-c4cccc5oc6c(-c7ccccc7)cccc6c45)c3)cc2)cc1 ; CC1(C)c2ccccc2-c2ccc(N(c3ccc(-c4ccccc4)cc3)c3ccccc3-c3ccccc3-c3cccc4oc5c(-c6ccccc6)cccc5c34)cc21 ; c1ccc(-c2ccc(N(c3ccc4c(c3)oc3ccccc34)c3ccccc3-c3ccccc3-c3cccc4oc5c(-c6ccccc6)cccc5c34)cc2)cc1]

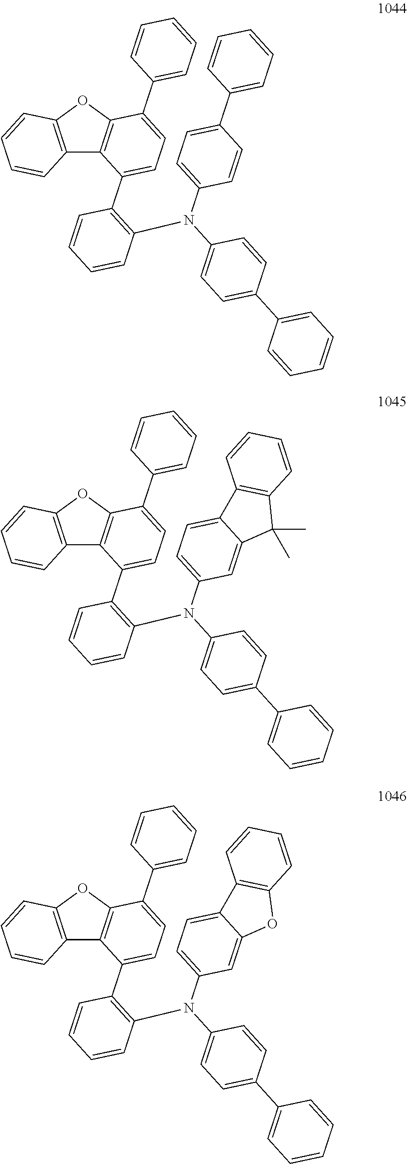 [SMILES: c1ccc(-c2ccc(N(c3ccc(-c4ccccc4)cc3)c3ccccc3-c3ccc(-c4ccccc4)c4oc5ccccc5c34)cc2)cc1 ; CC1(C)c2ccccc2-c2ccc(N(c3ccc(-c4ccccc4)cc3)c3ccccc3-c3ccc(-c4ccccc4)c4oc5ccccc5c34)cc21 ; c1ccc(-c2ccc(N(c3ccc4c(c3)oc3ccccc34)c3ccccc3-c3ccc(-c4ccccc4)c4oc5ccccc5c34)cc2)cc1]

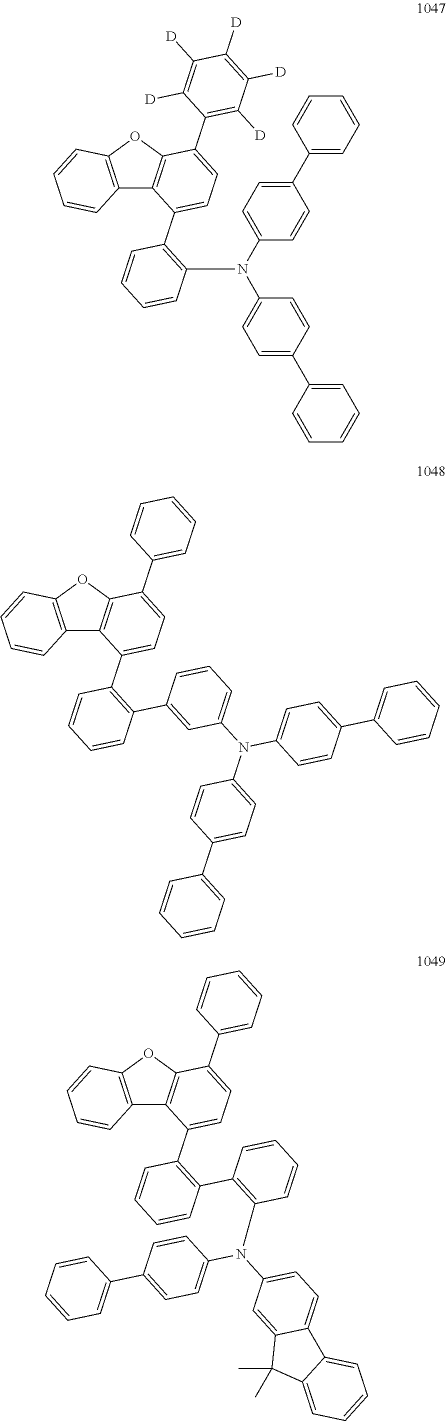 [SMILES: [2H]c1c([2H])c([2H])c(-c2ccc(-c3ccccc3N(c3ccc(-c4ccccc4)cc3)c3ccc(-c4ccccc4)cc3)c3c2oc2ccccc23)c([2H])c1[2H] ; c1ccc(-c2ccc(N(c3ccc(-c4ccccc4)cc3)c3cccc(-c4ccccc4-c4ccc(-c5ccccc5)c5oc6ccccc6c45)c3)cc2)cc1 ; CC1(C)c2ccccc2-c2ccc(N(c3ccc(-c4ccccc4)cc3)c3ccccc3-c3ccccc3-c3ccc(-c4ccccc4)c4oc5ccccc5c34)cc21]

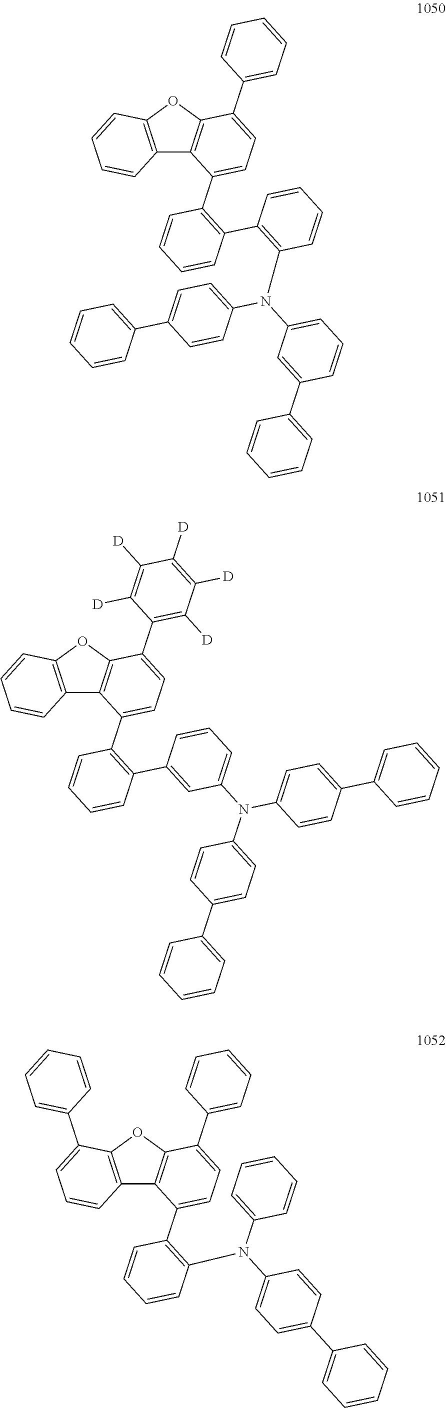 [SMILES: c1ccc(-c2ccc(N(c3cccc(-c4ccccc4)c3)c3ccccc3-c3ccccc3-c3ccc(-c4ccccc4)c4oc5ccccc5c34)cc2)cc1 ; [2H]c1c([2H])c([2H])c(-c2ccc(-c3ccccc3-c3cccc(N(c4ccc(-c5ccccc5)cc4)c4ccc(-c5ccccc5)cc4)c3)c3c2oc2ccccc23)c([2H])c1[2H] ; c1ccc(-c2ccc(N(c3ccccc3)c3ccccc3-c3ccc(-c4ccccc4)c4oc5c(-c6ccccc6)cccc5c34)cc2)cc1]

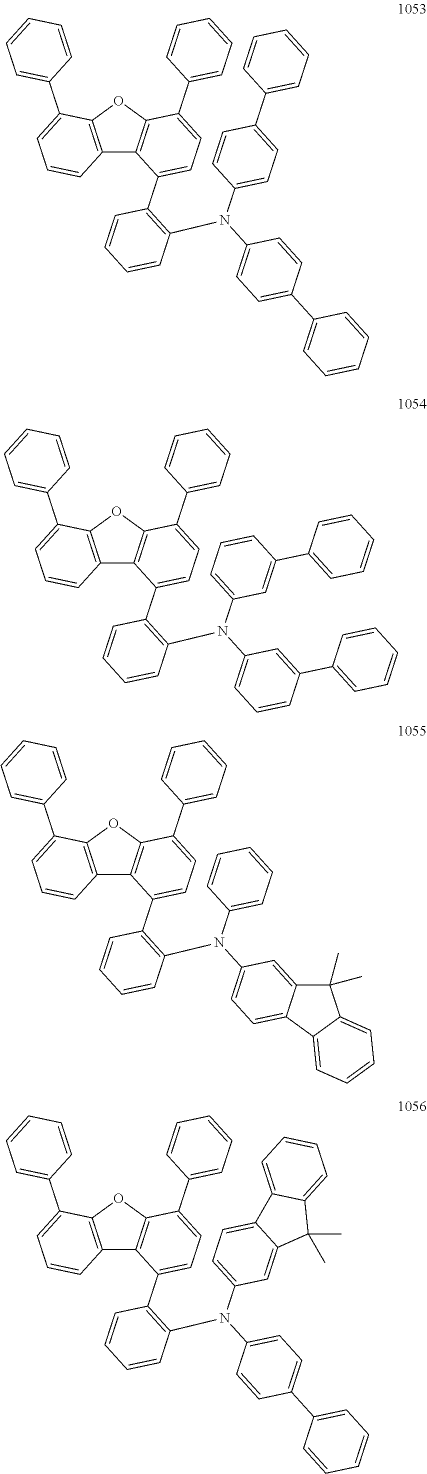 [SMILES: c1ccc(-c2ccc(N(c3ccc(-c4ccccc4)cc3)c3ccccc3-c3ccc(-c4ccccc4)c4oc5c(-c6ccccc6)cccc5c34)cc2)cc1 ; c1ccc(-c2cccc(N(c3cccc(-c4ccccc4)c3)c3ccccc3-c3ccc(-c4ccccc4)c4oc5c(-c6ccccc6)cccc5c34)c2)cc1 ; CC1(C)c2ccccc2-c2ccc(N(c3ccccc3)c3ccccc3-c3ccc(-c4ccccc4)c4oc5c(-c6ccccc6)cccc5c34)cc21 ; CC1(C)c2ccccc2-c2ccc(N(c3ccc(-c4ccccc4)cc3)c3ccccc3-c3ccc(-c4ccccc4)c4oc5c(-c6ccccc6)cccc5c34)cc21]

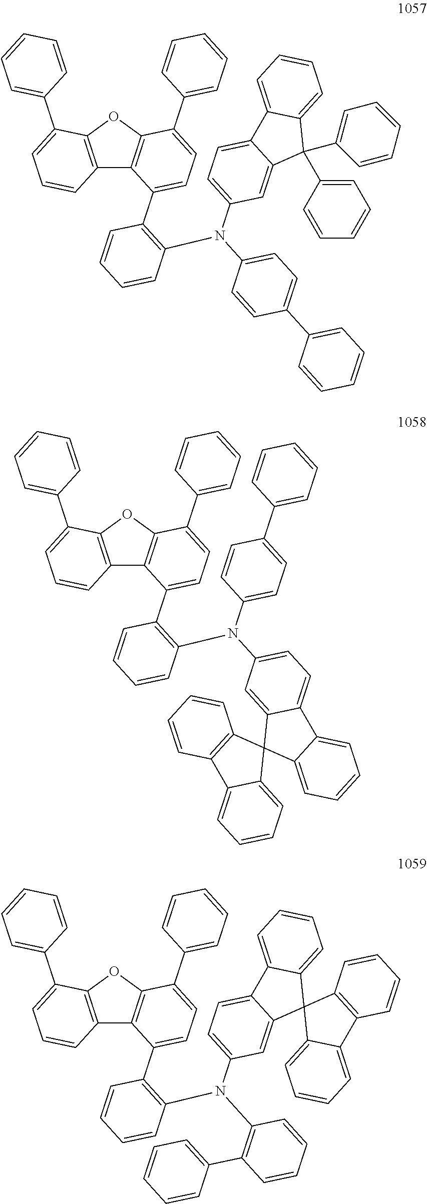 [SMILES: c1ccc(-c2ccc(N(c3ccc4c(c3)C(c3ccccc3)(c3ccccc3)c3ccccc3-4)c3ccccc3-c3ccc(-c4ccccc4)c4oc5c(-c6ccccc6)cccc5c34)cc2)cc1 ; c1ccc(-c2ccc(N(c3ccc4c(c3)C3(c5ccccc5-c5ccccc53)c3ccccc3-4)c3ccccc3-c3ccc(-c4ccccc4)c4oc5c(-c6ccccc6)cccc5c34)cc2)cc1 ; c1ccc(-c2ccccc2N(c2ccc3c(c2)C2(c4ccccc4-c4ccccc42)c2ccccc2-3)c2ccccc2-c2ccc(-c3ccccc3)c3oc4c(-c5ccccc5)cccc4c23)cc1]

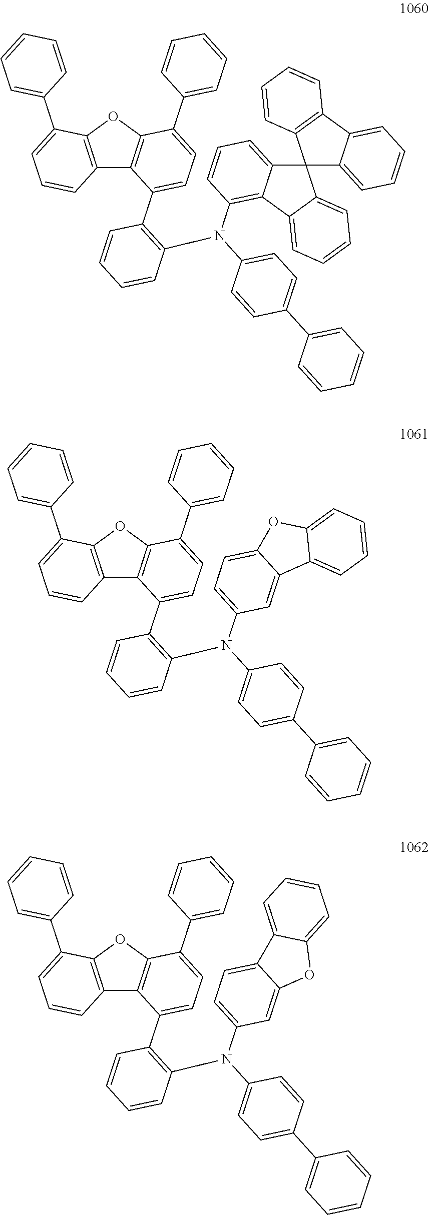 [SMILES: c1ccc(-c2ccc(N(c3ccccc3-c3ccc(-c4ccccc4)c4oc5c(-c6ccccc6)cccc5c34)c3cccc4c3-c3ccccc3C43c4ccccc4-c4ccccc43)cc2)cc1 ; c1ccc(-c2ccc(N(c3ccc4oc5ccccc5c4c3)c3ccccc3-c3ccc(-c4ccccc4)c4oc5c(-c6ccccc6)cccc5c34)cc2)cc1 ; c1ccc(-c2ccc(N(c3ccc4c(c3)oc3ccccc34)c3ccccc3-c3ccc(-c4ccccc4)c4oc5c(-c6ccccc6)cccc5c34)cc2)cc1]

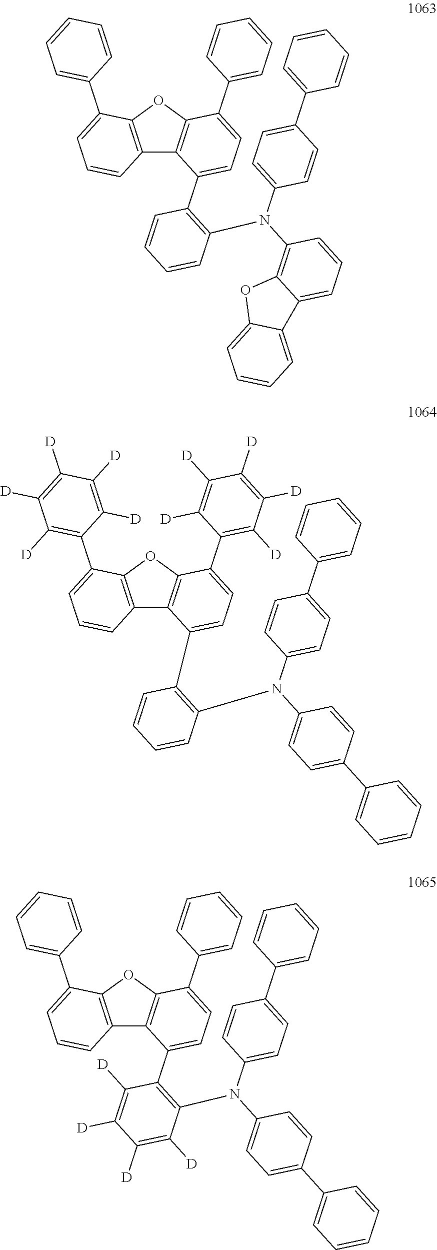 [SMILES: c1ccc(-c2ccc(N(c3ccccc3-c3ccc(-c4ccccc4)c4oc5c(-c6ccccc6)cccc5c34)c3cccc4c3oc3ccccc34)cc2)cc1 ; [2H]c1c([2H])c([2H])c(-c2cccc3c2oc2c(-c4c([2H])c([2H])c([2H])c([2H])c4[2H])ccc(-c4ccccc4N(c4ccc(-c5ccccc5)cc4)c4ccc(-c5ccccc5)cc4)c23)c([2H])c1[2H] ; [2H]c1c([2H])c([2H])c(N(c2ccc(-c3ccccc3)cc2)c2ccc(-c3ccccc3)cc2)c(-c2ccc(-c3ccccc3)c3oc4c(-c5ccccc5)cccc4c23)c1[2H]]

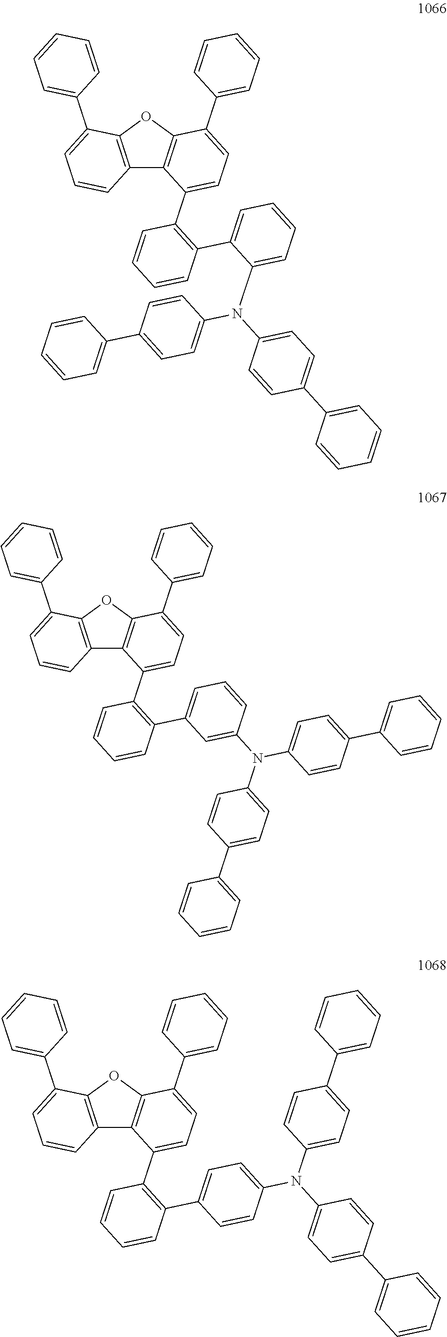 [SMILES: c1ccc(-c2ccc(N(c3ccc(-c4ccccc4)cc3)c3ccccc3-c3ccccc3-c3ccc(-c4ccccc4)c4oc5c(-c6ccccc6)cccc5c34)cc2)cc1 ; c1ccc(-c2ccc(N(c3ccc(-c4ccccc4)cc3)c3cccc(-c4ccccc4-c4ccc(-c5ccccc5)c5oc6c(-c7ccccc7)cccc6c45)c3)cc2)cc1 ; c1ccc(-c2ccc(N(c3ccc(-c4ccccc4)cc3)c3ccc(-c4ccccc4-c4ccc(-c5ccccc5)c5oc6c(-c7ccccc7)cccc6c45)cc3)cc2)cc1]

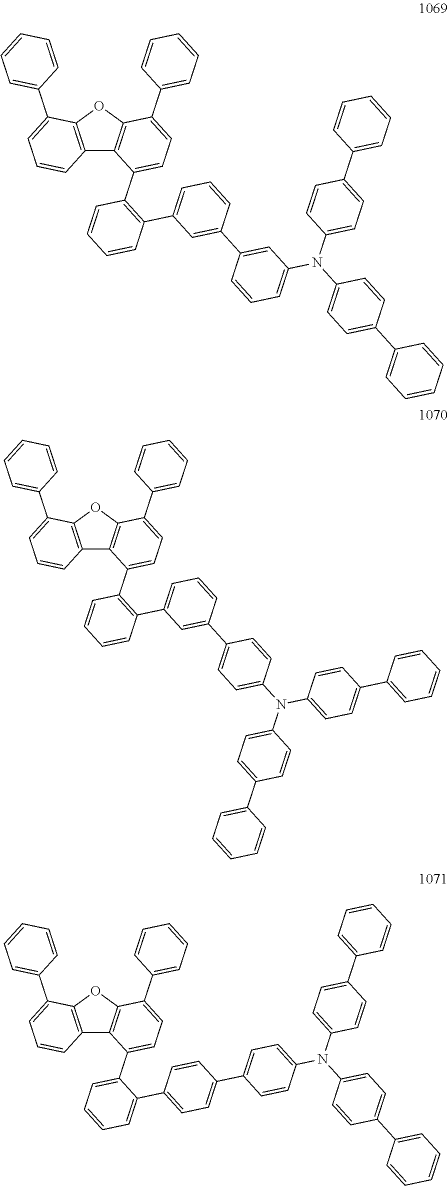 [SMILES: c1ccc(-c2ccc(N(c3ccc(-c4ccccc4)cc3)c3cccc(-c4cccc(-c5ccccc5-c5ccc(-c6ccccc6)c6oc7c(-c8ccccc8)cccc7c56)c4)c3)cc2)cc1 ; c1ccc(-c2ccc(N(c3ccc(-c4ccccc4)cc3)c3ccc(-c4cccc(-c5ccccc5-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4ccc(-c5ccccc5-c5ccc(-c6ccccc6)c6oc7c(-c8ccccc8)cccc7c56)cc4)cc3)cc2)cc1]

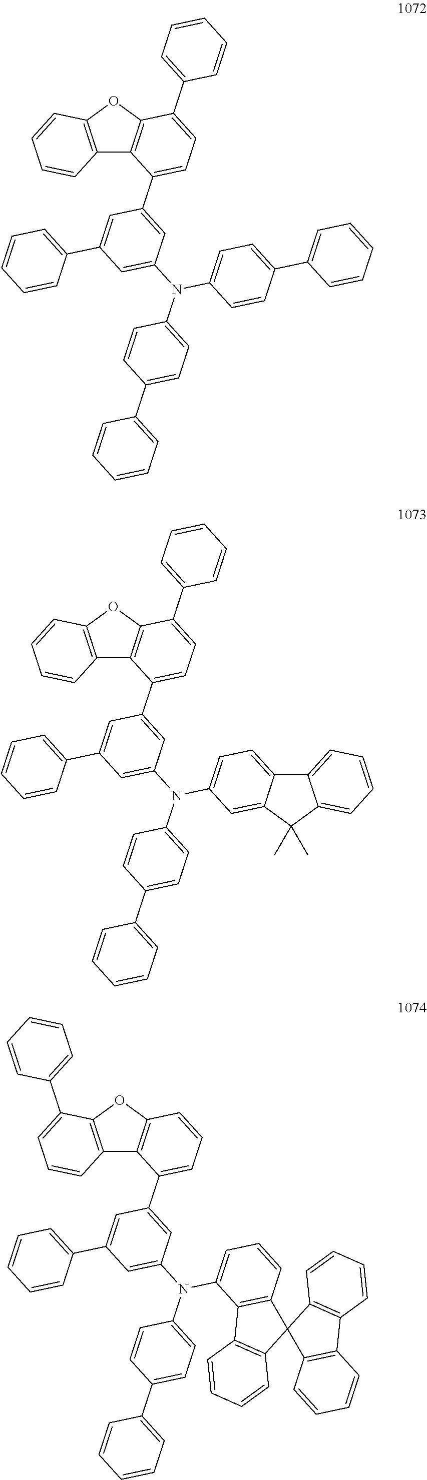 [SMILES: c1ccc(-c2ccc(N(c3ccc(-c4ccccc4)cc3)c3cc(-c4ccccc4)cc(-c4ccc(-c5ccccc5)c5oc6ccccc6c45)c3)cc2)cc1 ; CC1(C)c2ccccc2-c2ccc(N(c3ccc(-c4ccccc4)cc3)c3cc(-c4ccccc4)cc(-c4ccc(-c5ccccc5)c5oc6ccccc6c45)c3)cc21 ; c1ccc(-c2ccc(N(c3cc(-c4ccccc4)cc(-c4cccc5oc6c(-c7ccccc7)cccc6c45)c3)c3cccc4c3-c3ccccc3C43c4ccccc4-c4ccccc43)cc2)cc1]

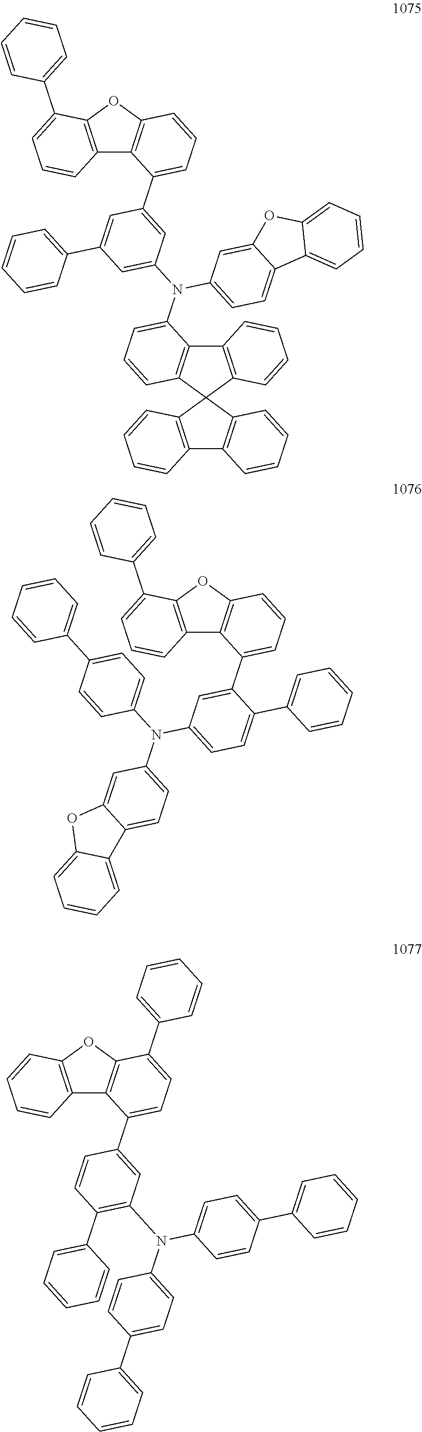

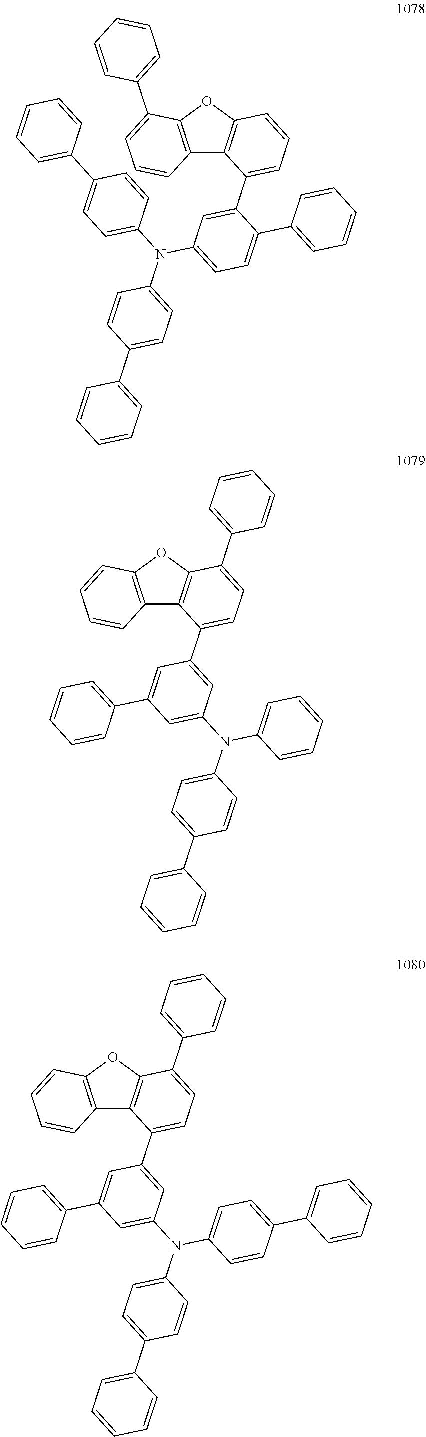 [SMILES: c1ccc(-c2ccc(N(c3ccc(-c4ccccc4)cc3)c3ccc(-c4ccccc4)c(-c4cccc5oc6c(-c7ccccc7)cccc6c45)c3)cc2)cc1 ; c1ccc(-c2ccc(N(c3ccccc3)c3cc(-c4ccccc4)cc(-c4ccc(-c5ccccc5)c5oc6ccccc6c45)c3)cc2)cc1 ; c1ccc(-c2ccc(N(c3ccc(-c4ccccc4)cc3)c3cc(-c4ccccc4)cc(-c4ccc(-c5ccccc5)c5oc6ccccc6c45)c3)cc2)cc1]

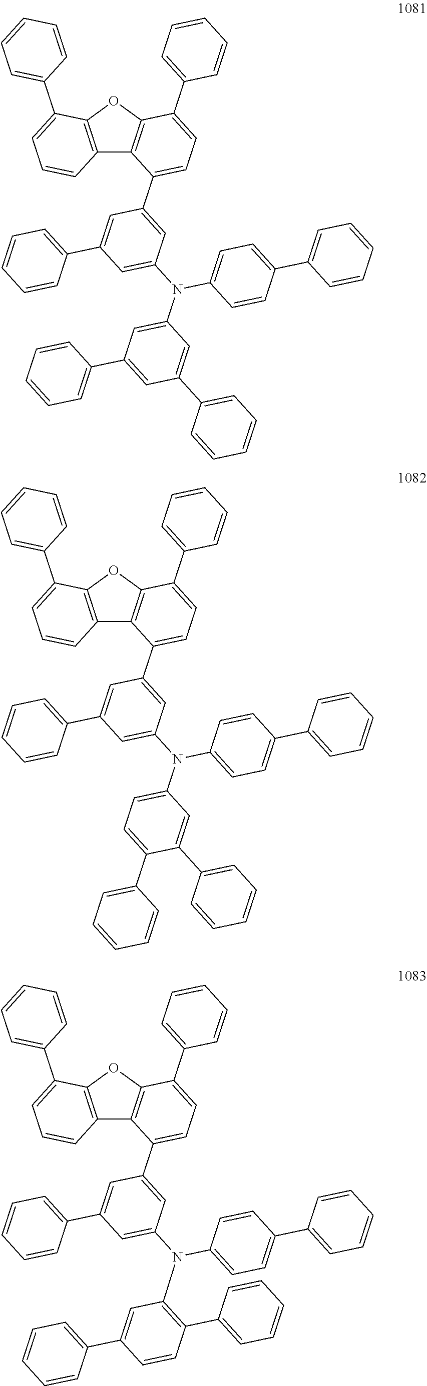

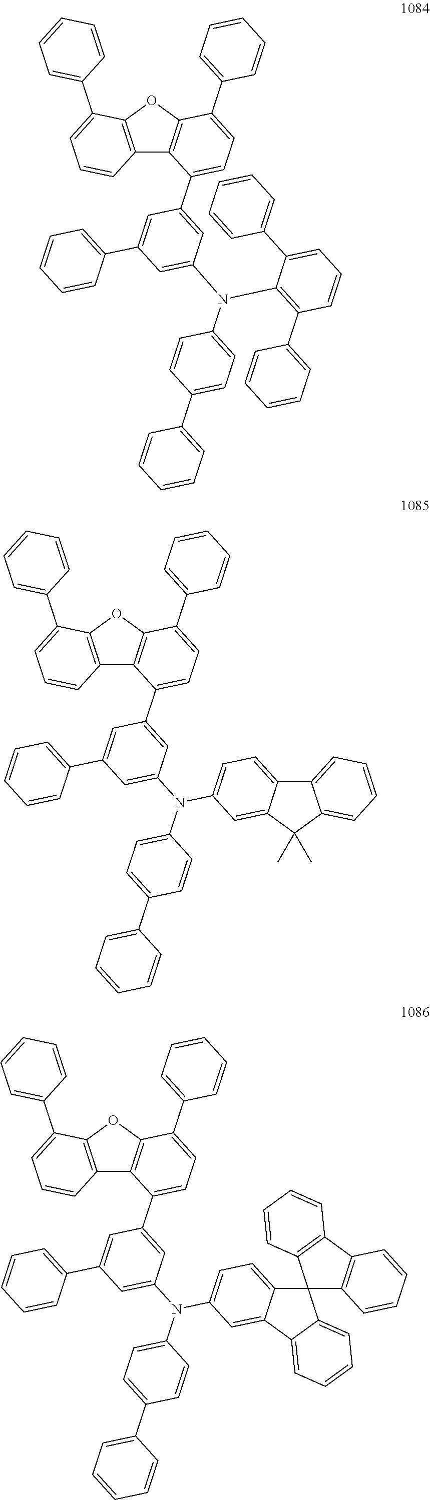 [SMILES: c1ccc(-c2ccc(N(c3cc(-c4ccccc4)cc(-c4ccc(-c5ccccc5)c5oc6c(-c7ccccc7)cccc6c45)c3)c3c(-c4ccccc4)cccc3-c3ccccc3)cc2)cc1 ; CC1(C)c2ccccc2-c2ccc(N(c3ccc(-c4ccccc4)cc3)c3cc(-c4ccccc4)cc(-c4ccc(-c5ccccc5)c5oc6c(-c7ccccc7)cccc6c45)c3)cc21 ; c1ccc(-c2ccc(N(c3cc(-c4ccccc4)cc(-c4ccc(-c5ccccc5)c5oc6c(-c7ccccc7)cccc6c45)c3)c3ccc4c(c3)-c3ccccc3C43c4ccccc4-c4ccccc43)cc2)cc1]

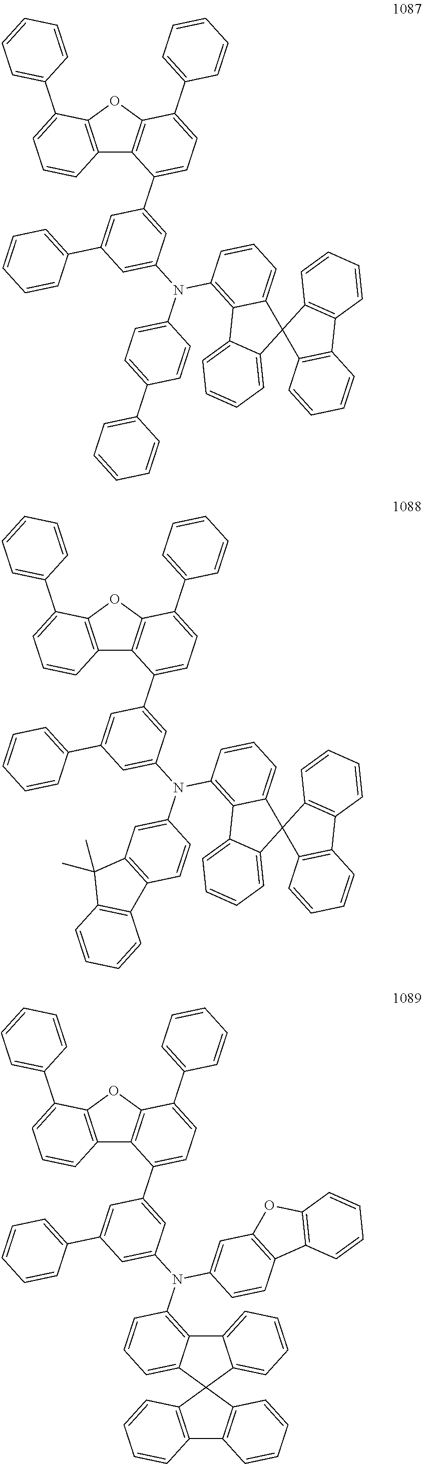 [SMILES: c1ccc(-c2ccc(N(c3cc(-c4ccccc4)cc(-c4ccc(-c5ccccc5)c5oc6c(-c7ccccc7)cccc6c45)c3)c3cccc4c3-c3ccccc3C43c4ccccc4-c4ccccc43)cc2)cc1 ; CC1(C)c2ccccc2-c2ccc(N(c3cc(-c4ccccc4)cc(-c4ccc(-c5ccccc5)c5oc6c(-c7ccccc7)cccc6c45)c3)c3cccc4c3-c3ccccc3C43c4ccccc4-c4ccccc43)cc21 ; c1ccc(-c2cc(-c3ccc(-c4ccccc4)c4oc5c(-c6ccccc6)cccc5c34)cc(N(c3ccc4c(c3)oc3ccccc34)c3cccc4c3-c3ccccc3C43c4ccccc4-c4ccccc43)c2)cc1]

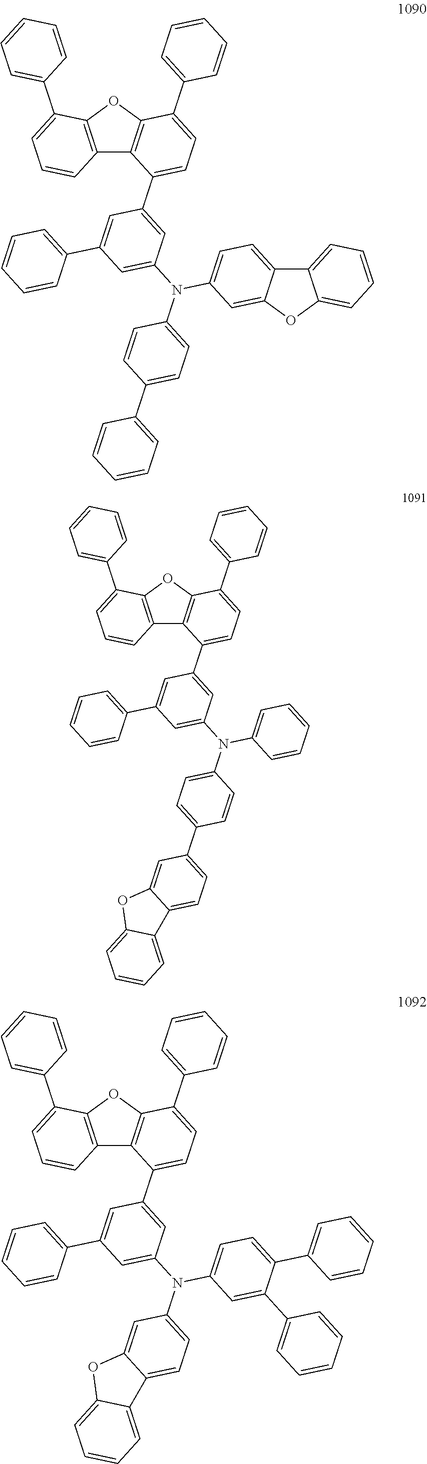

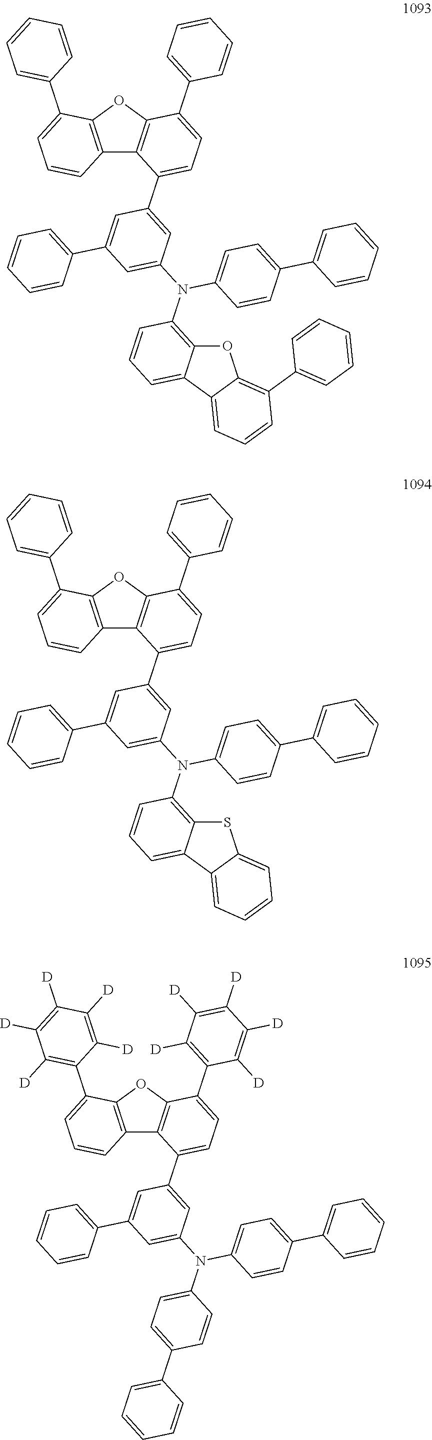 [SMILES: c1ccc(-c2ccc(N(c3cc(-c4ccccc4)cc(-c4ccc(-c5ccccc5)c5oc6c(-c7ccccc7)cccc6c45)c3)c3cccc4c3oc3c(-c5ccccc5)cccc34)cc2)cc1 ; c1ccc(-c2ccc(N(c3cc(-c4ccccc4)cc(-c4ccc(-c5ccccc5)c5oc6c(-c7ccccc7)cccc6c45)c3)c3cccc4c3sc3ccccc34)cc2)cc1 ; [2H]c1c([2H])c([2H])c(-c2cccc3c2oc2c(-c4c([2H])c([2H])c([2H])c([2H])c4[2H])ccc(-c4cc(-c5ccccc5)cc(N(c5ccc(-c6ccccc6)cc5)c5ccc(-c6ccccc6)cc5)c4)c23)c([2H])c1[2H]]

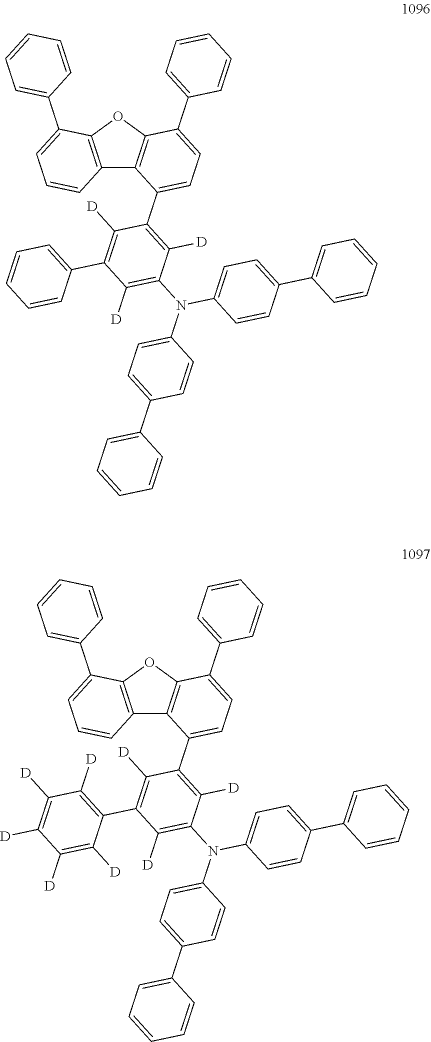 [SMILES: [2H]c1c(-c2ccccc2)c([2H])c(N(c2ccc(-c3ccccc3)cc2)c2ccc(-c3ccccc3)cc2)c([2H])c1-c1ccc(-c2ccccc2)c2oc3c(-c4ccccc4)cccc3c12 ; [2H]c1c([2H])c([2H])c(-c2c([2H])c(-c3ccc(-c4ccccc4)c4oc5c(-c6ccccc6)cccc5c34)c([2H])c(N(c3ccc(-c4ccccc4)cc3)c3ccc(-c4ccccc4)cc3)c2[2H])c([2H])c1[2H]]

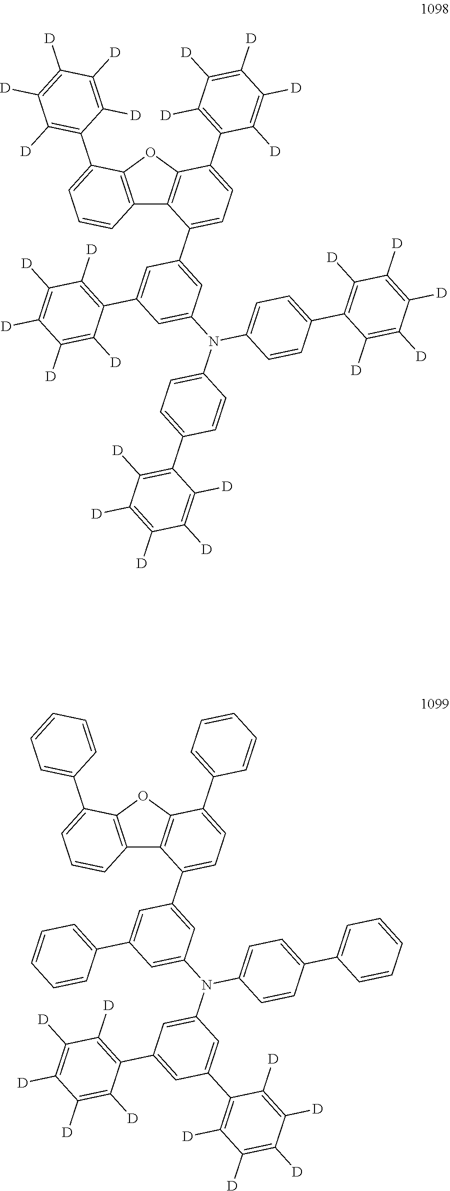 [SMILES: [2H]c1c([2H])c([2H])c(-c2ccc(N(c3ccc(-c4c([2H])c([2H])c([2H])c([2H])c4[2H])cc3)c3cc(-c4c([2H])c([2H])c([2H])c([2H])c4[2H])cc(-c4ccc(-c5c([2H])c([2H])c([2H])c([2H])c5[2H])c5oc6c(-c7c([2H])c([2H])c([2H])c([2H])c7[2H])cccc6c45)c3)cc2)c([2H])c1[2H] ; [2H]c1c([2H])c([2H])c(-c2cc(-c3c([2H])c([2H])c([2H])c([2H])c3[2H])cc(N(c3ccc(-c4ccccc4)cc3)c3cc(-c4ccccc4)cc(-c4ccc(-c5ccccc5)c5oc6c(-c7ccccc7)cccc6c45)c3)c2)c([2H])c1[2H]]

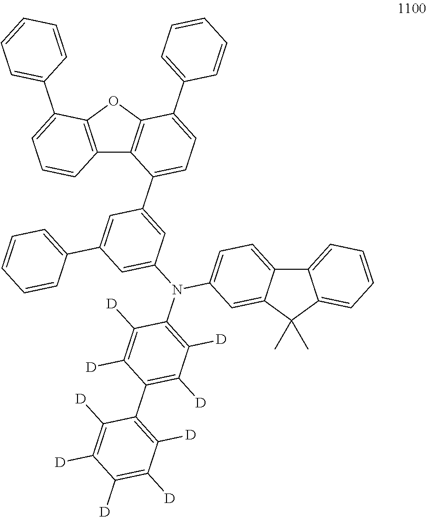 [SMILES: [2H]c1c([2H])c([2H])c(-c2c([2H])c([2H])c(N(c3cc(-c4ccccc4)cc(-c4ccc(-c5ccccc5)c5oc6c(-c7ccccc7)cccc6c45)c3)c3ccc4c(c3)C(C)(C)c3ccccc3-4)c([2H])c2[2H])c([2H])c1[2H]]

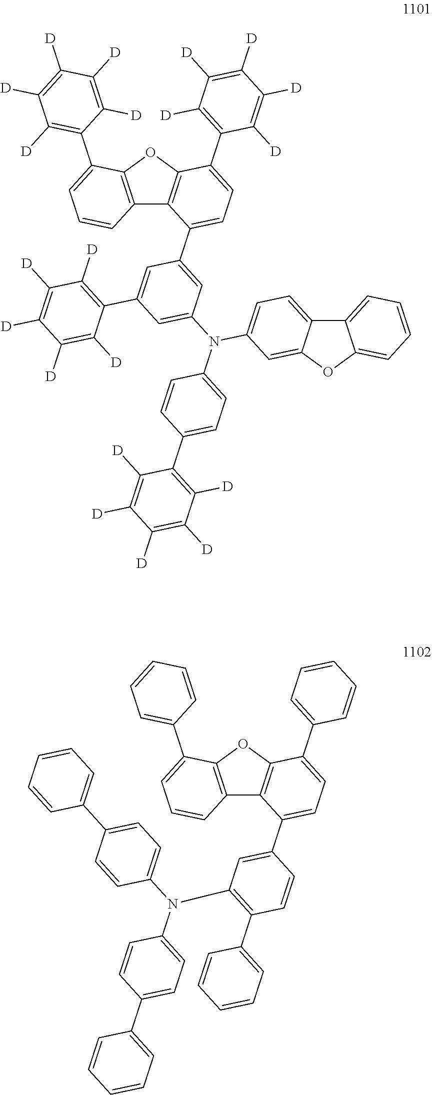 [SMILES: [2H]c1c([2H])c([2H])c(-c2ccc(N(c3cc(-c4c([2H])c([2H])c([2H])c([2H])c4[2H])cc(-c4ccc(-c5c([2H])c([2H])c([2H])c([2H])c5[2H])c5oc6c(-c7c([2H])c([2H])c([2H])c([2H])c7[2H])cccc6c45)c3)c3ccc4c(c3)oc3ccccc34)cc2)c([2H])c1[2H] ; c1ccc(-c2ccc(N(c3ccc(-c4ccccc4)cc3)c3cc(-c4ccc(-c5ccccc5)c5oc6c(-c7ccccc7)cccc6c45)ccc3-c3ccccc3)cc2)cc1]

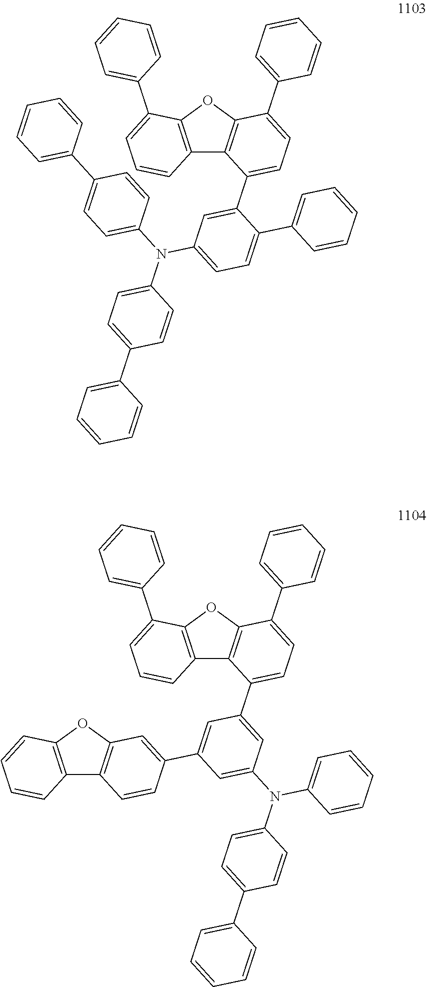 [SMILES: c1ccc(-c2ccc(N(c3ccc(-c4ccccc4)cc3)c3ccc(-c4ccccc4)c(-c4ccc(-c5ccccc5)c5oc6c(-c7ccccc7)cccc6c45)c3)cc2)cc1 ; c1ccc(-c2ccc(N(c3ccccc3)c3cc(-c4ccc5c(c4)oc4ccccc45)cc(-c4ccc(-c5ccccc5)c5oc6c(-c7ccccc7)cccc6c45)c3)cc2)cc1]

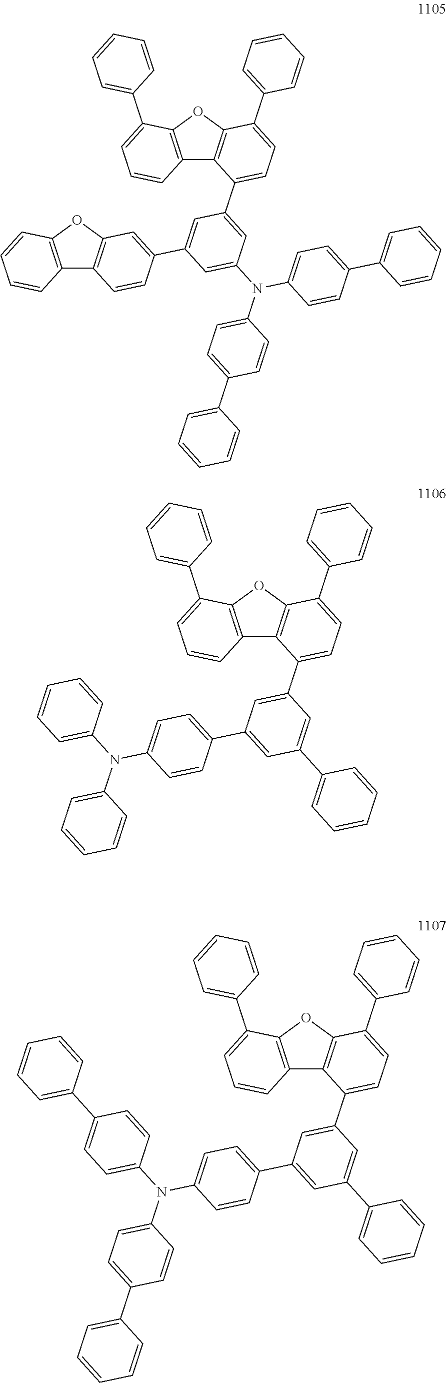

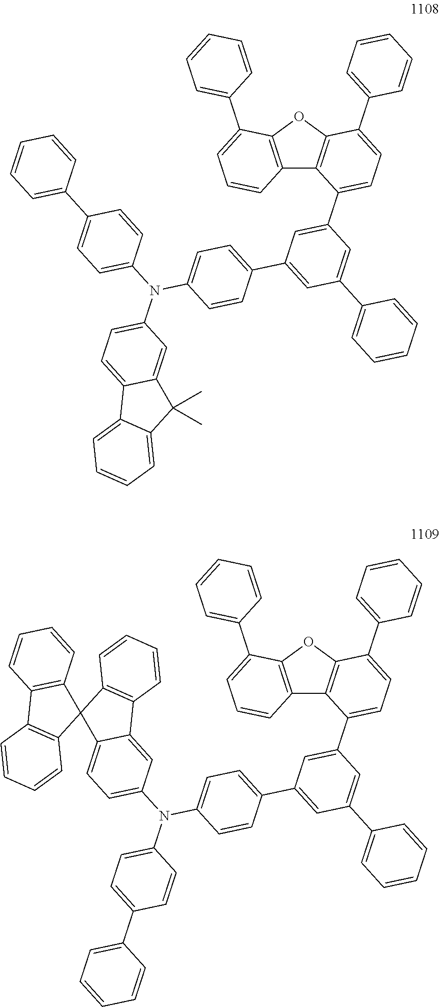 [SMILES: CC1(C)c2ccccc2-c2ccc(N(c3ccc(-c4ccccc4)cc3)c3ccc(-c4cc(-c5ccccc5)cc(-c5ccc(-c6ccccc6)c6oc7c(-c8ccccc8)cccc7c56)c4)cc3)cc21 ; c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc4c(c3)-c3ccccc3C43c4ccccc4-c4ccccc43)cc2)cc1]

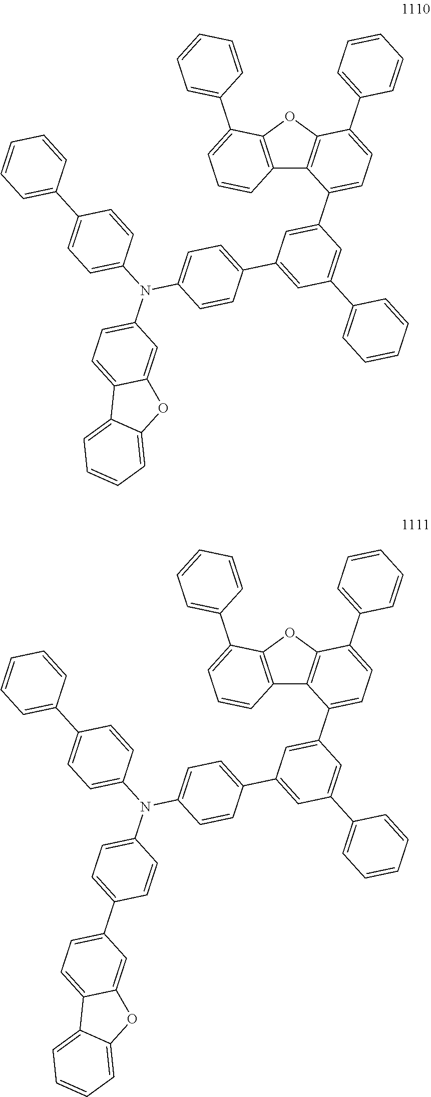 [SMILES: c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc4c(c3)oc3ccccc34)cc2)cc1 ; c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc(-c4ccc5c(c4)oc4ccccc45)cc3)cc2)cc1]

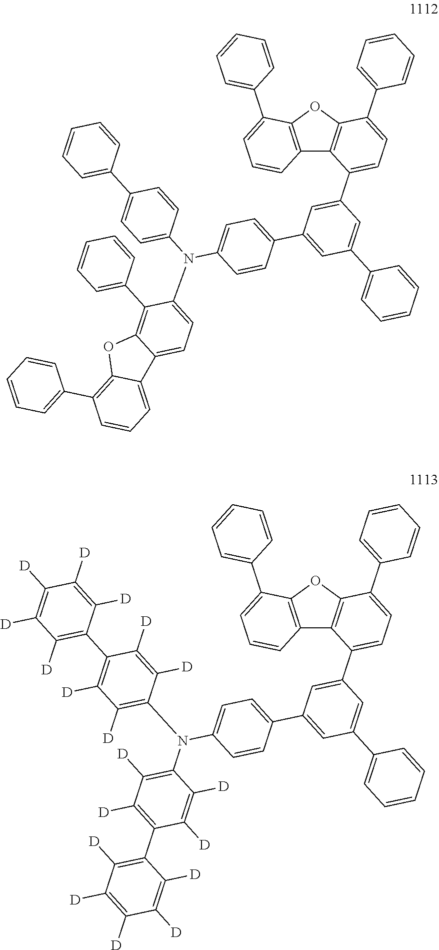 [SMILES: c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc4c(oc5c(-c6ccccc6)cccc54)c3-c3ccccc3)cc2)cc1 ; [2H]c1c([2H])c([2H])c(-c2c([2H])c([2H])c(N(c3ccc(-c4cc(-c5ccccc5)cc(-c5ccc(-c6ccccc6)c6oc7c(-c8ccccc8)cccc7c56)c4)cc3)c3c([2H])c([2H])c(-c4c([2H])c([2H])c([2H])c([2H])c4[2H])c([2H])c3[2H])c([2H])c2[2H])c([2H])c1[2H]]

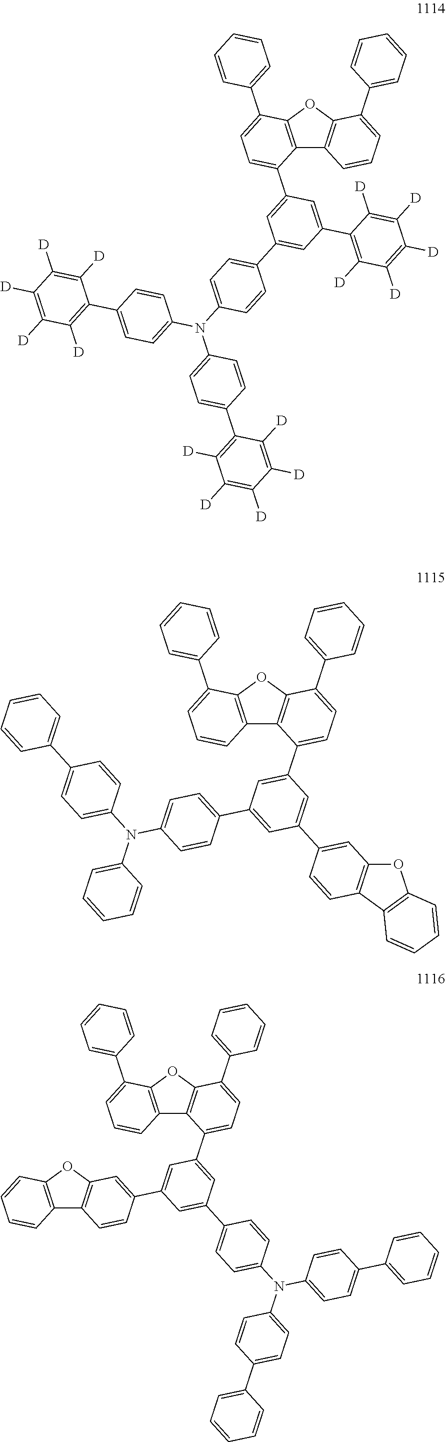 [SMILES: [2H]c1c([2H])c([2H])c(-c2ccc(N(c3ccc(-c4cc(-c5c([2H])c([2H])c([2H])c([2H])c5[2H])cc(-c5ccc(-c6ccccc6)c6oc7c(-c8ccccc8)cccc7c56)c4)cc3)c3ccc(-c4c([2H])c([2H])c([2H])c([2H])c4[2H])cc3)cc2)c([2H])c1[2H] ; c1ccc(-c2ccc(N(c3ccccc3)c3ccc(-c4cc(-c5ccc6c(c5)oc5ccccc56)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5ccc6c(c5)oc5ccccc56)cc(-c5ccc(-c6ccccc6)c6oc7c(-c8ccccc8)cccc7c56)c4)cc3)cc2)cc1]

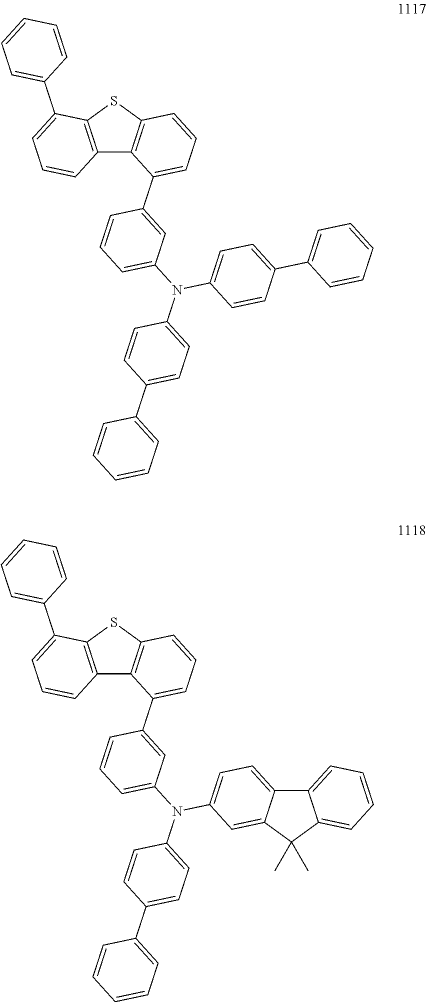 [SMILES: c1ccc(-c2ccc(N(c3ccc(-c4ccccc4)cc3)c3cccc(-c4cccc5sc6c(-c7ccccc7)cccc6c45)c3)cc2)cc1 ; CC1(C)c2ccccc2-c2ccc(N(c3ccc(-c4ccccc4)cc3)c3cccc(-c4cccc5sc6c(-c7ccccc7)cccc6c45)c3)cc21]

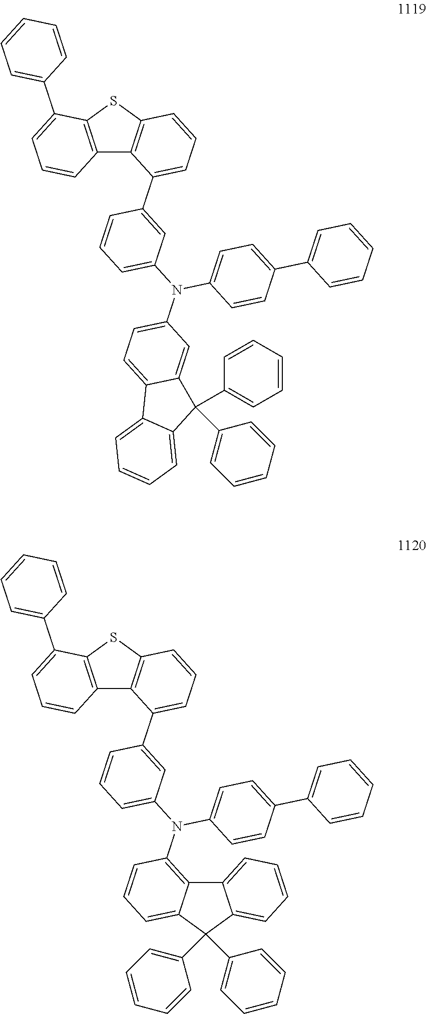 [SMILES: c1ccc(-c2ccc(N(c3cccc(-c4cccc5sc6c(-c7ccccc7)cccc6c45)c3)c3ccc4c(c3)C(c3ccccc3)(c3ccccc3)c3ccccc3-4)cc2)cc1 ; c1ccc(-c2ccc(N(c3cccc(-c4cccc5sc6c(-c7ccccc7)cccc6c45)c3)c3cccc4c3-c3ccccc3C4(c3ccccc3)c3ccccc3)cc2)cc1]

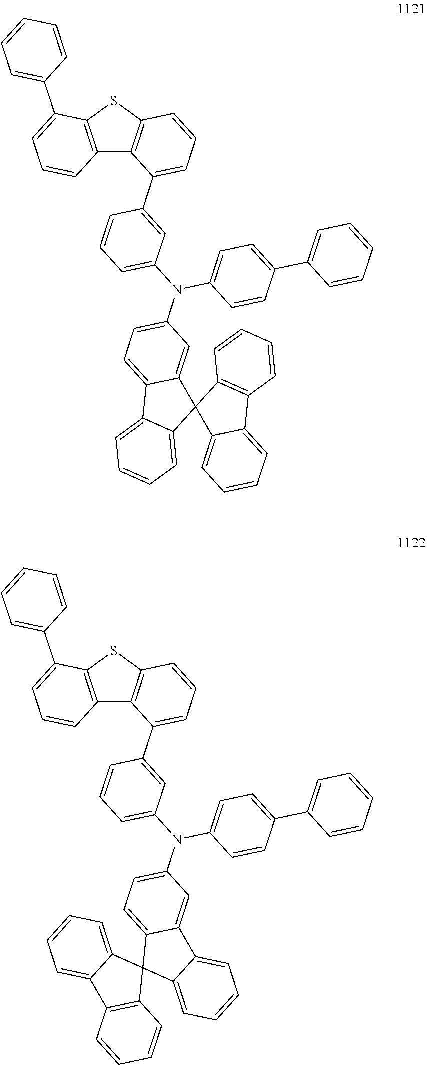 [SMILES: c1ccc(-c2ccc(N(c3cccc(-c4cccc5sc6c(-c7ccccc7)cccc6c45)c3)c3ccc4c(c3)C3(c5ccccc5-c5ccccc53)c3ccccc3-4)cc2)cc1 ; c1ccc(-c2ccc(N(c3cccc(-c4cccc5sc6c(-c7ccccc7)cccc6c45)c3)c3ccc4c(c3)-c3ccccc3C43c4ccccc4-c4ccccc43)cc2)cc1]

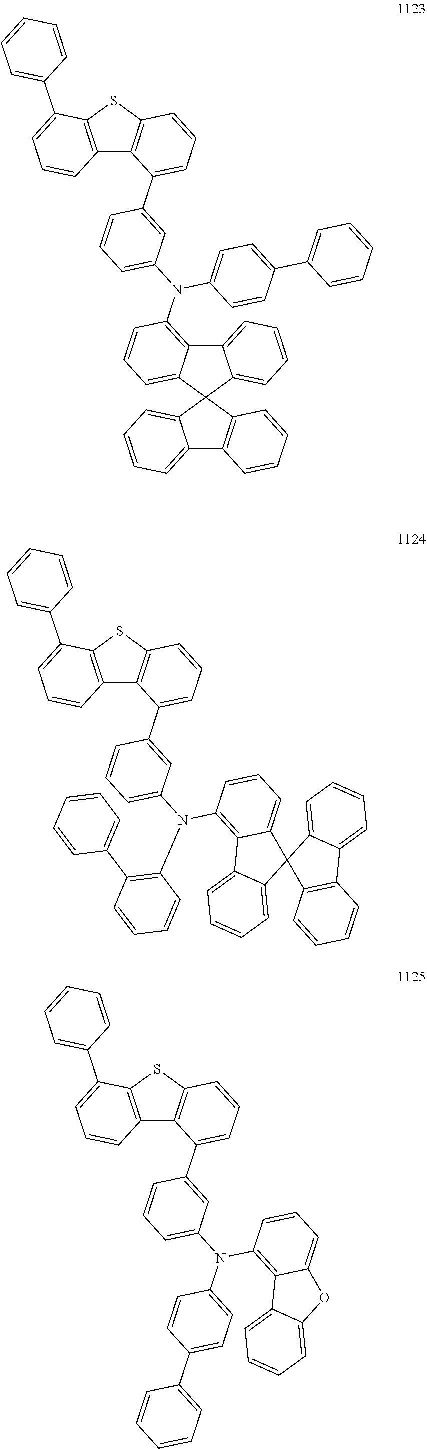 [SMILES: c1ccc(-c2ccc(N(c3cccc(-c4cccc5sc6c(-c7ccccc7)cccc6c45)c3)c3cccc4c3-c3ccccc3C43c4ccccc4-c4ccccc43)cc2)cc1 ; c1ccc(-c2ccccc2N(c2cccc(-c3cccc4sc5c(-c6ccccc6)cccc5c34)c2)c2cccc3c2-c2ccccc2C32c3ccccc3-c3ccccc32)cc1 ; c1ccc(-c2ccc(N(c3cccc(-c4cccc5sc6c(-c7ccccc7)cccc6c45)c3)c3cccc4oc5ccccc5c34)cc2)cc1]

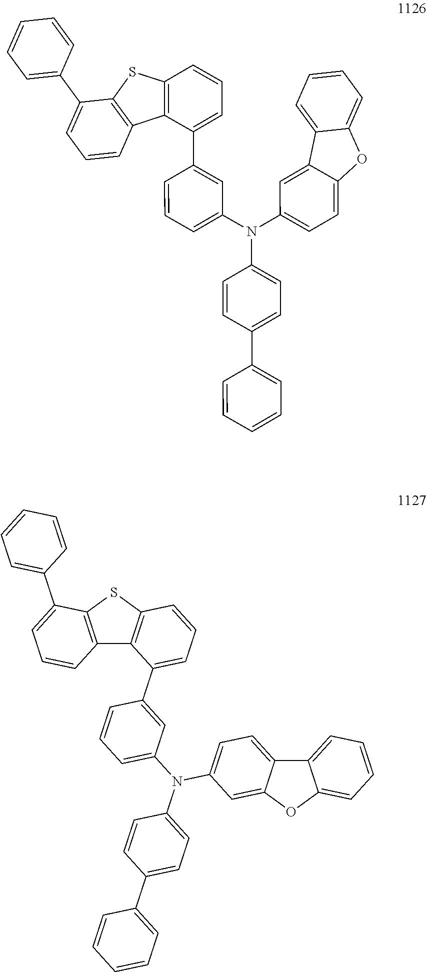 [SMILES: c1ccc(-c2ccc(N(c3cccc(-c4cccc5sc6c(-c7ccccc7)cccc6c45)c3)c3ccc4oc5ccccc5c4c3)cc2)cc1 ; c1ccc(-c2ccc(N(c3cccc(-c4cccc5sc6c(-c7ccccc7)cccc6c45)c3)c3ccc4c(c3)oc3ccccc34)cc2)cc1]

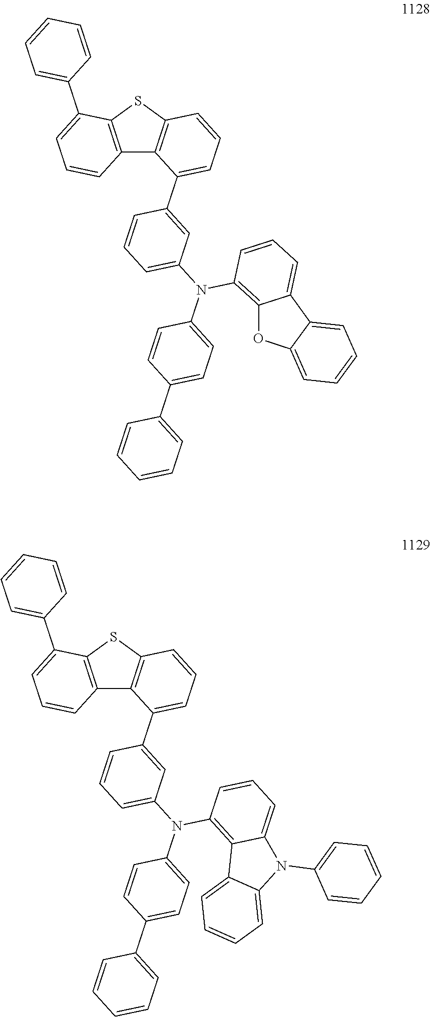 [SMILES: c1ccc(-c2ccc(N(c3cccc(-c4cccc5sc6c(-c7ccccc7)cccc6c45)c3)c3cccc4c3oc3ccccc34)cc2)cc1 ; c1ccc(-c2ccc(N(c3cccc(-c4cccc5sc6c(-c7ccccc7)cccc6c45)c3)c3cccc4c3c3ccccc3n4-c3ccccc3)cc2)cc1]

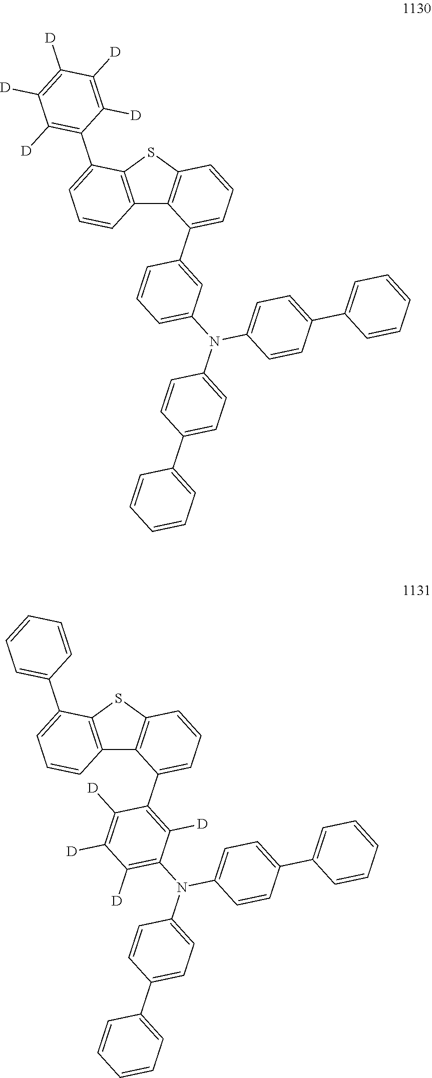 [SMILES: [2H]c1c([2H])c([2H])c(-c2cccc3c2sc2cccc(-c4cccc(N(c5ccc(-c6ccccc6)cc5)c5ccc(-c6ccccc6)cc5)c4)c23)c([2H])c1[2H] ; [2H]c1c([2H])c(-c2cccc3sc4c(-c5ccccc5)cccc4c23)c([2H])c(N(c2ccc(-c3ccccc3)cc2)c2ccc(-c3ccccc3)cc2)c1[2H]]

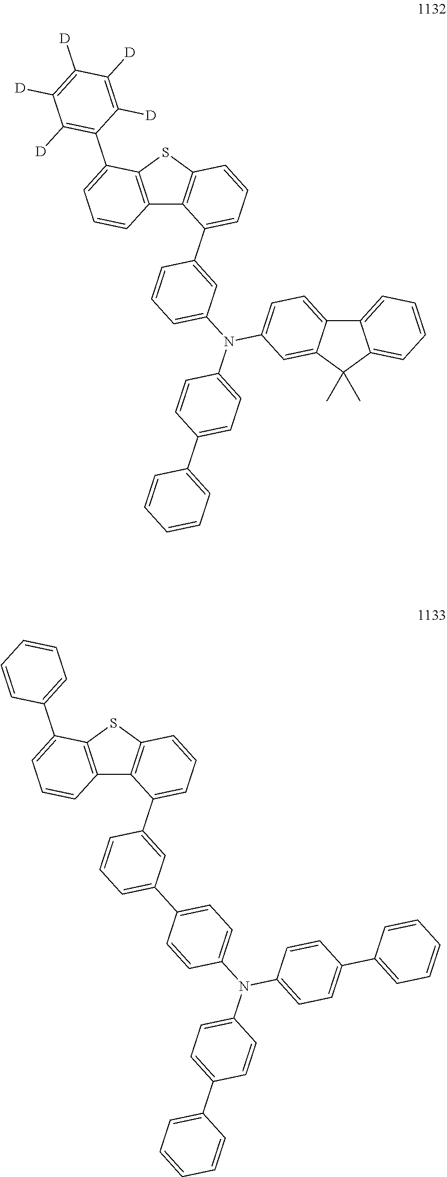 [SMILES: [2H]c1c([2H])c([2H])c(-c2cccc3c2sc2cccc(-c4cccc(N(c5ccc(-c6ccccc6)cc5)c5ccc6c(c5)C(C)(C)c5ccccc5-6)c4)c23)c([2H])c1[2H] ; c1ccc(-c2ccc(N(c3ccc(-c4ccccc4)cc3)c3ccc(-c4cccc(-c5cccc6sc7c(-c8ccccc8)cccc7c56)c4)cc3)cc2)cc1]

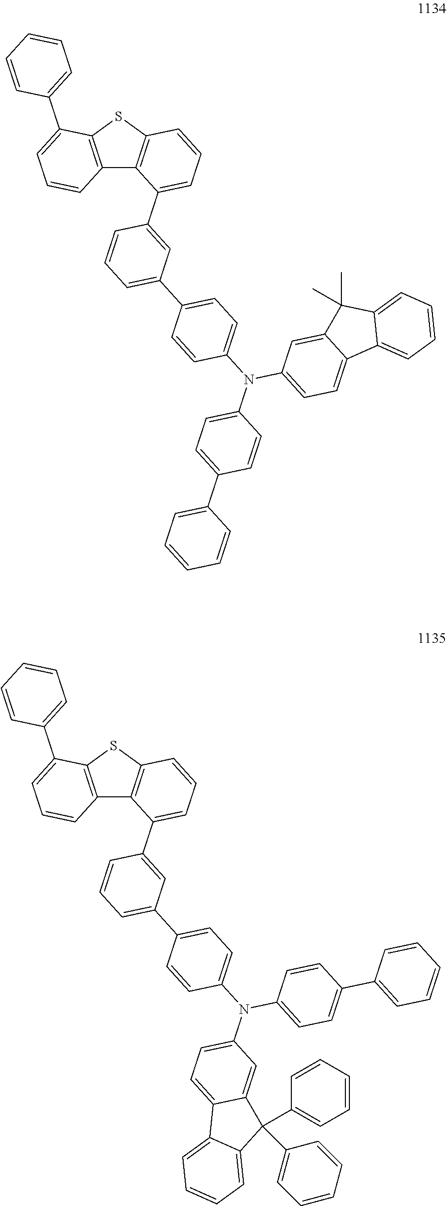 [SMILES: CC1(C)c2ccccc2-c2ccc(N(c3ccc(-c4ccccc4)cc3)c3ccc(-c4cccc(-c5cccc6sc7c(-c8ccccc8)cccc7c56)c4)cc3)cc21 ; c1ccc(-c2ccc(N(c3ccc(-c4cccc(-c5cccc6sc7c(-c8ccccc8)cccc7c56)c4)cc3)c3ccc4c(c3)C(c3ccccc3)(c3ccccc3)c3ccccc3-4)cc2)cc1]

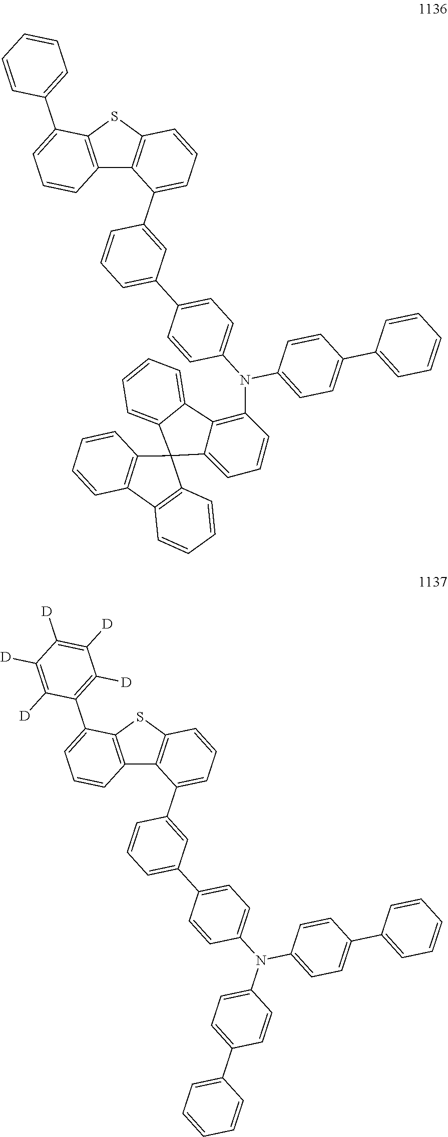 [SMILES: c1ccc(-c2ccc(N(c3ccc(-c4cccc(-c5cccc6sc7c(-c8ccccc8)cccc7c56)c4)cc3)c3cccc4c3-c3ccccc3C43c4ccccc4-c4ccccc43)cc2)cc1 ; [2H]c1c([2H])c([2H])c(-c2cccc3c2sc2cccc(-c4cccc(-c5ccc(N(c6ccc(-c7ccccc7)cc6)c6ccc(-c7ccccc7)cc6)cc5)c4)c23)c([2H])c1[2H]]

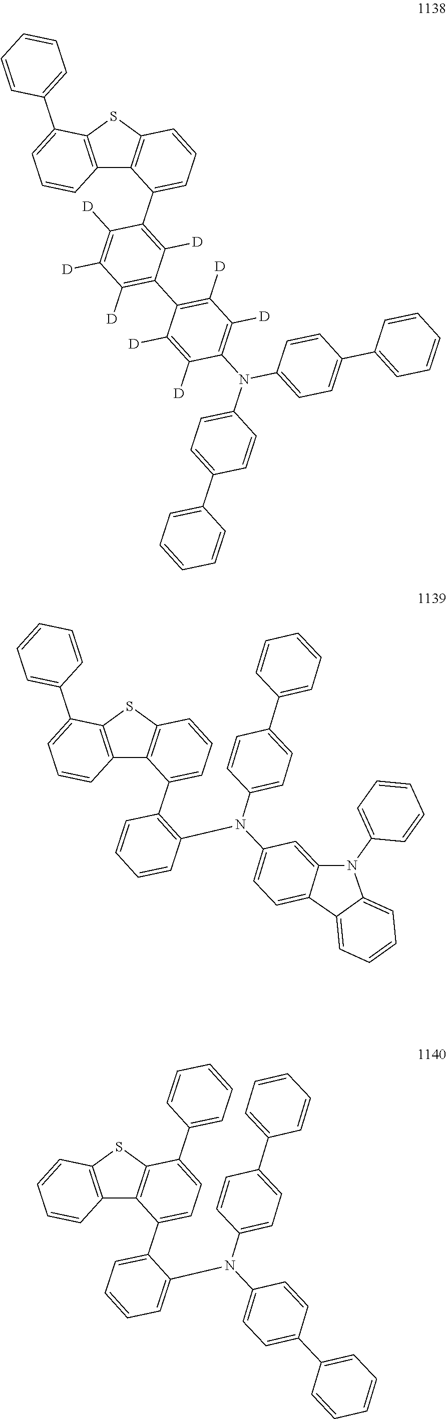 [SMILES: [2H]c1c([2H])c(-c2c([2H])c([2H])c(N(c3ccc(-c4ccccc4)cc3)c3ccc(-c4ccccc4)cc3)c([2H])c2[2H])c([2H])c(-c2cccc3sc4c(-c5ccccc5)cccc4c23)c1[2H] ; c1ccc(-c2ccc(N(c3ccc4c5ccccc5n(-c5ccccc5)c4c3)c3ccccc3-c3cccc4sc5c(-c6ccccc6)cccc5c34)cc2)cc1 ; c1ccc(-c2ccc(N(c3ccc(-c4ccccc4)cc3)c3ccccc3-c3ccc(-c4ccccc4)c4sc5ccccc5c34)cc2)cc1]

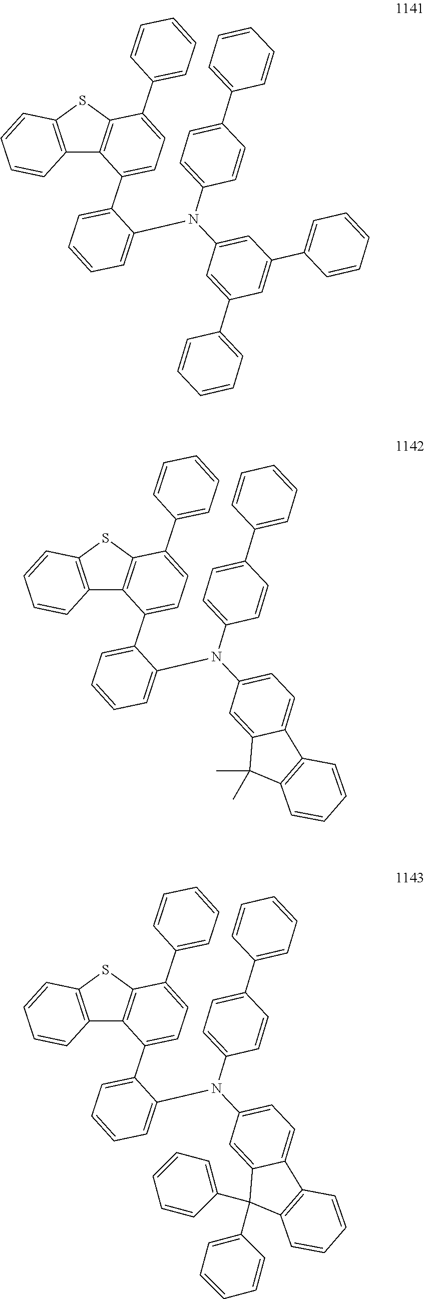 [SMILES: c1ccc(-c2ccc(N(c3cc(-c4ccccc4)cc(-c4ccccc4)c3)c3ccccc3-c3ccc(-c4ccccc4)c4sc5ccccc5c34)cc2)cc1 ; CC1(C)c2ccccc2-c2ccc(N(c3ccc(-c4ccccc4)cc3)c3ccccc3-c3ccc(-c4ccccc4)c4sc5ccccc5c34)cc21 ; c1ccc(-c2ccc(N(c3ccc4c(c3)C(c3ccccc3)(c3ccccc3)c3ccccc3-4)c3ccccc3-c3ccc(-c4ccccc4)c4sc5ccccc5c34)cc2)cc1]

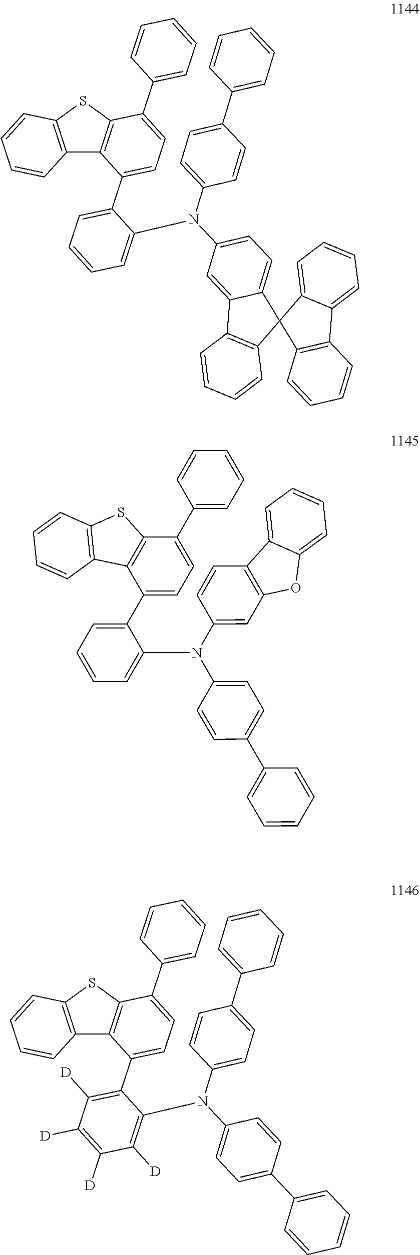 [SMILES: c1ccc(-c2ccc(N(c3ccc4c(c3)-c3ccccc3C43c4ccccc4-c4ccccc43)c3ccccc3-c3ccc(-c4ccccc4)c4sc5ccccc5c34)cc2)cc1 ; c1ccc(-c2ccc(N(c3ccc4c(c3)oc3ccccc34)c3ccccc3-c3ccc(-c4ccccc4)c4sc5ccccc5c34)cc2)cc1 ; [2H]c1c([2H])c([2H])c(N(c2ccc(-c3ccccc3)cc2)c2ccc(-c3ccccc3)cc2)c(-c2ccc(-c3ccccc3)c3sc4ccccc4c23)c1[2H]]

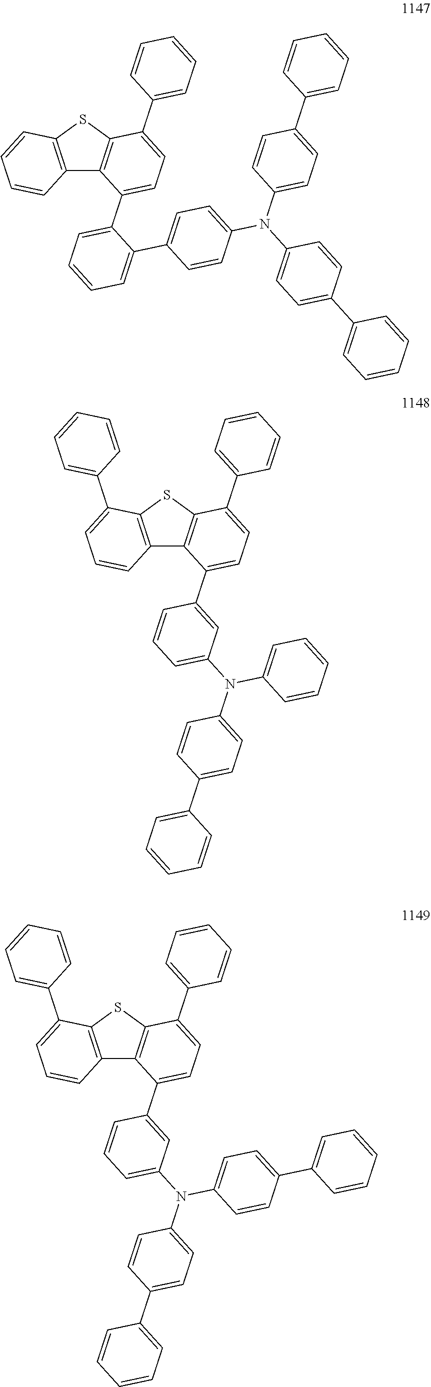 [SMILES: c1ccc(-c2ccc(N(c3ccc(-c4ccccc4)cc3)c3ccc(-c4ccccc4-c4ccc(-c5ccccc5)c5sc6ccccc6c45)cc3)cc2)cc1 ; c1ccc(-c2ccc(N(c3ccccc3)c3cccc(-c4ccc(-c5ccccc5)c5sc6c(-c7ccccc7)cccc6c45)c3)cc2)cc1 ; c1ccc(-c2ccc(N(c3ccc(-c4ccccc4)cc3)c3cccc(-c4ccc(-c5ccccc5)c5sc6c(-c7ccccc7)cccc6c45)c3)cc2)cc1]

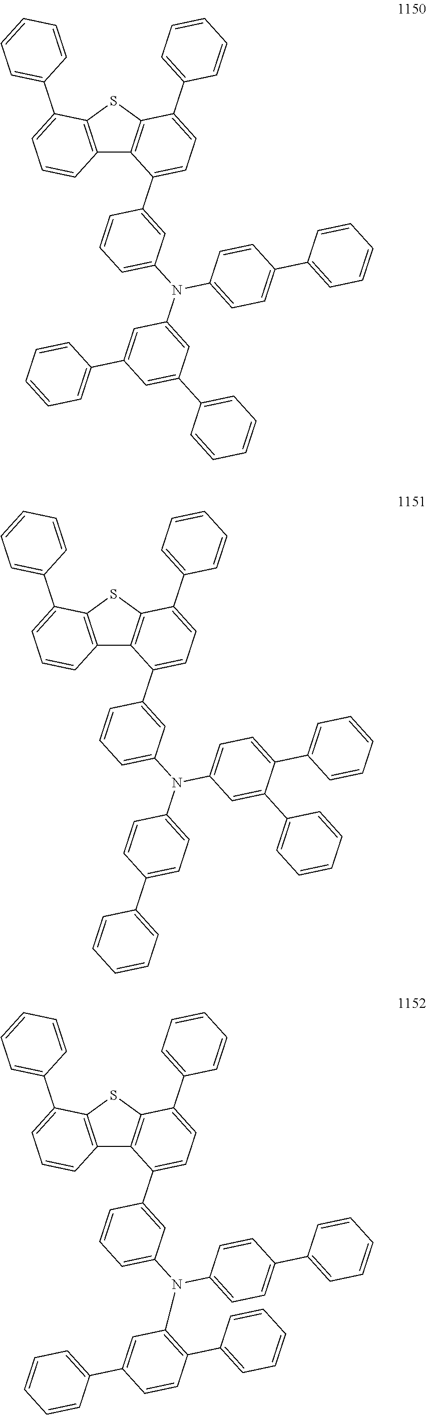 [SMILES: c1ccc(-c2ccc(N(c3cc(-c4ccccc4)cc(-c4ccccc4)c3)c3cccc(-c4ccc(-c5ccccc5)c5sc6c(-c7ccccc7)cccc6c45)c3)cc2)cc1 ; c1ccc(-c2ccc(N(c3cccc(-c4ccc(-c5ccccc5)c5sc6c(-c7ccccc7)cccc6c45)c3)c3ccc(-c4ccccc4)c(-c4ccccc4)c3)cc2)cc1 ; c1ccc(-c2ccc(N(c3cccc(-c4ccc(-c5ccccc5)c5sc6c(-c7ccccc7)cccc6c45)c3)c3cc(-c4ccccc4)ccc3-c3ccccc3)cc2)cc1]

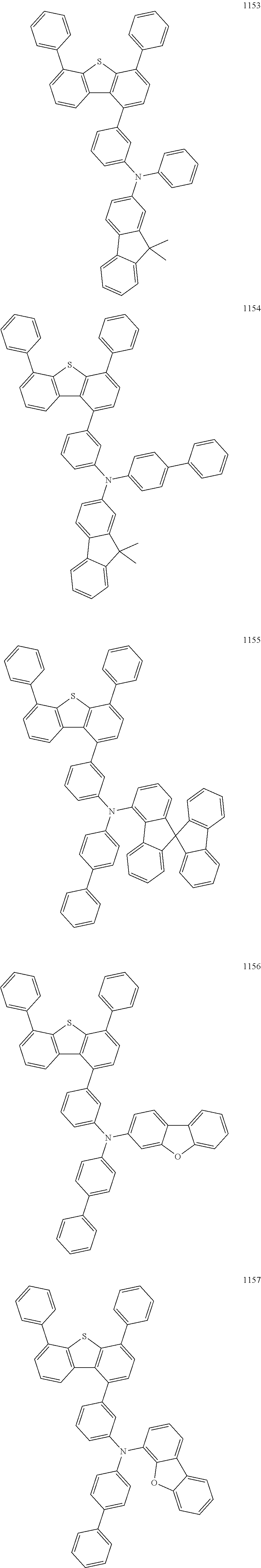

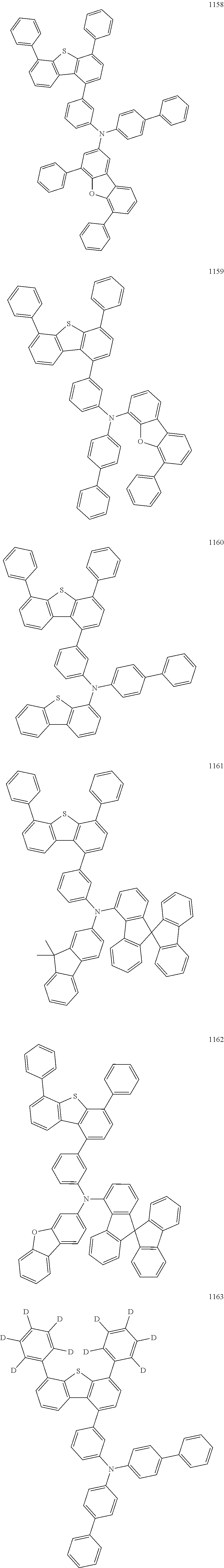

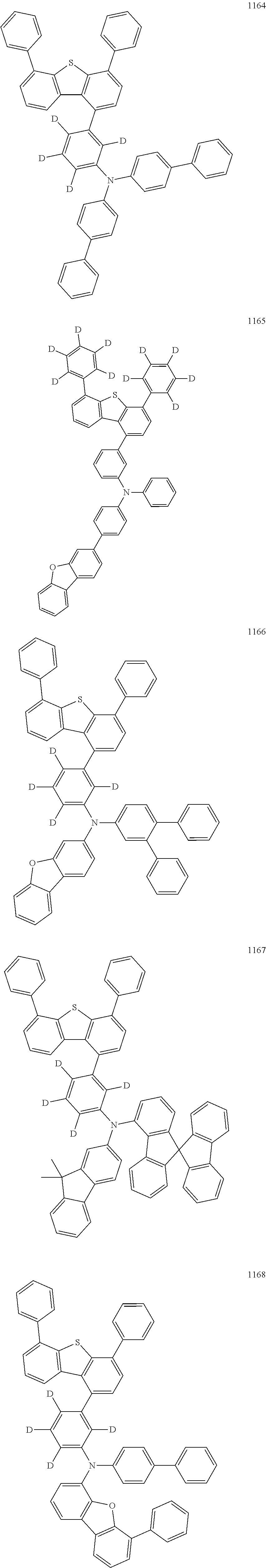

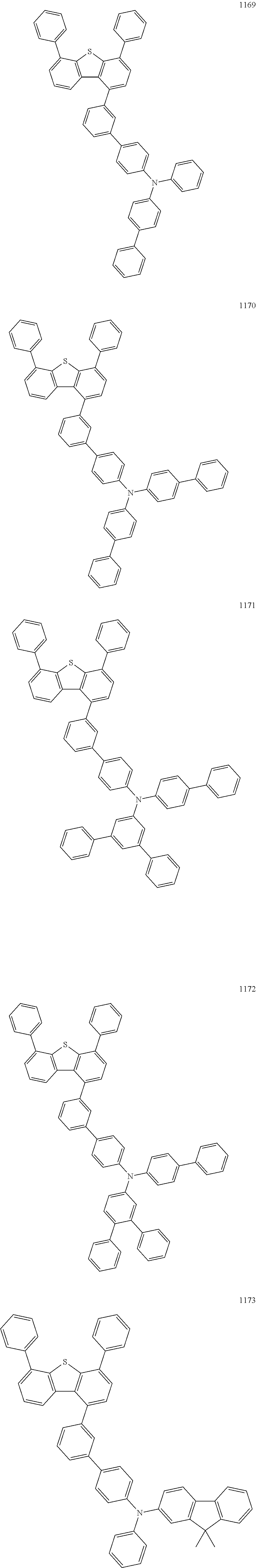

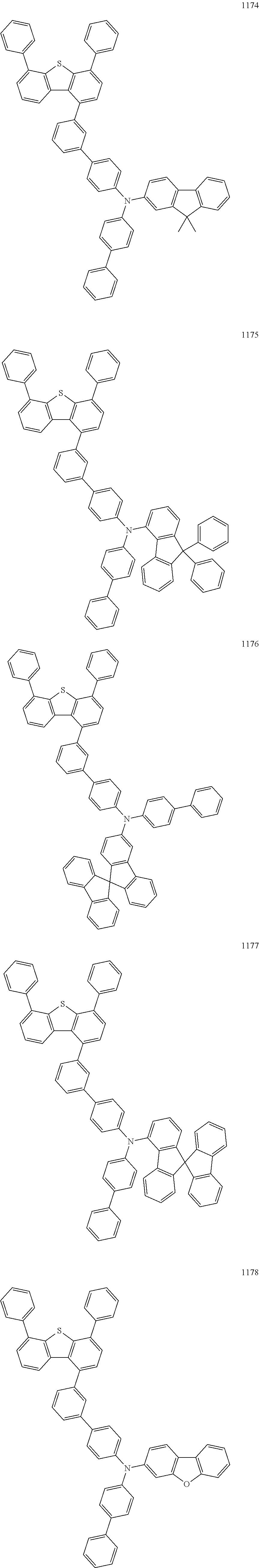

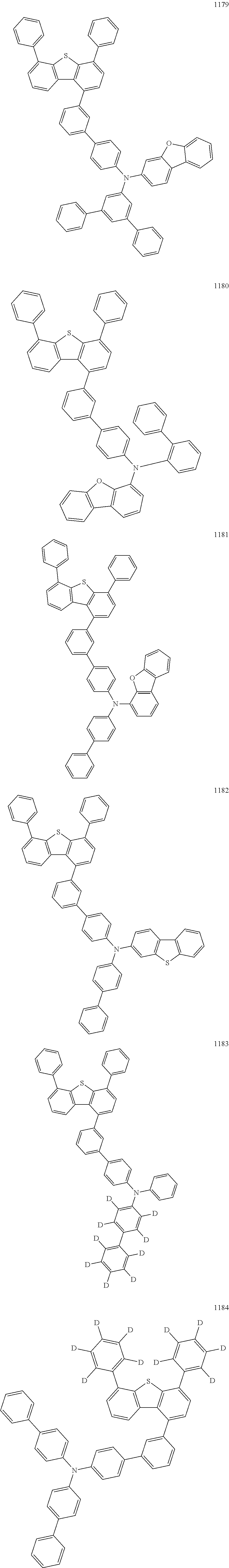

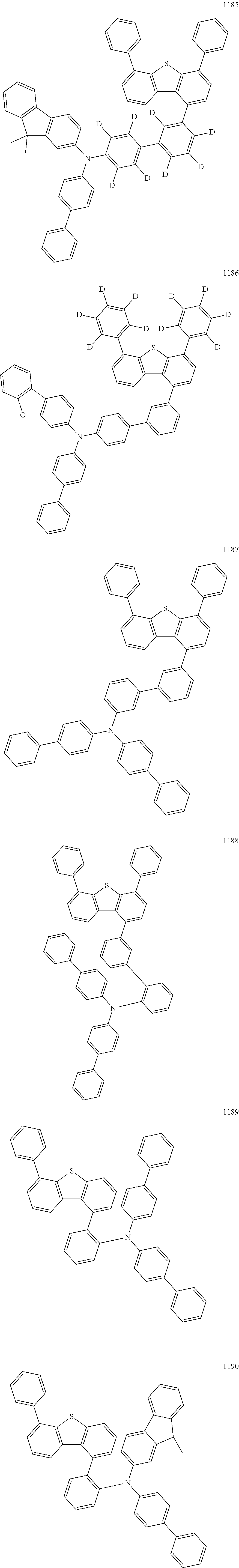

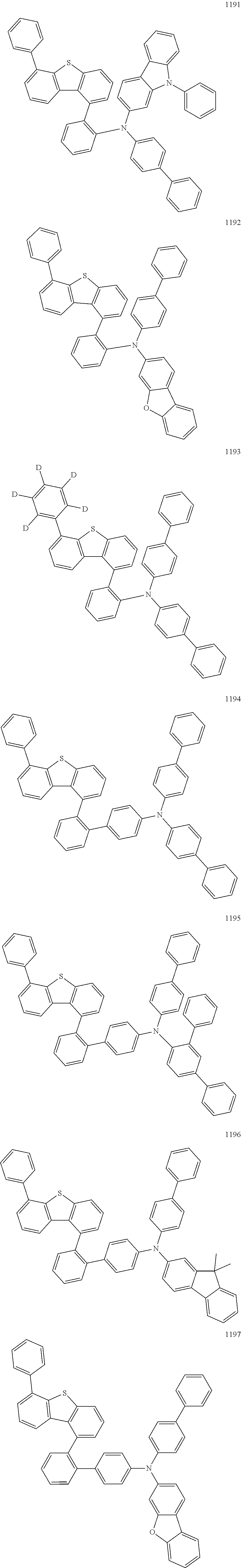

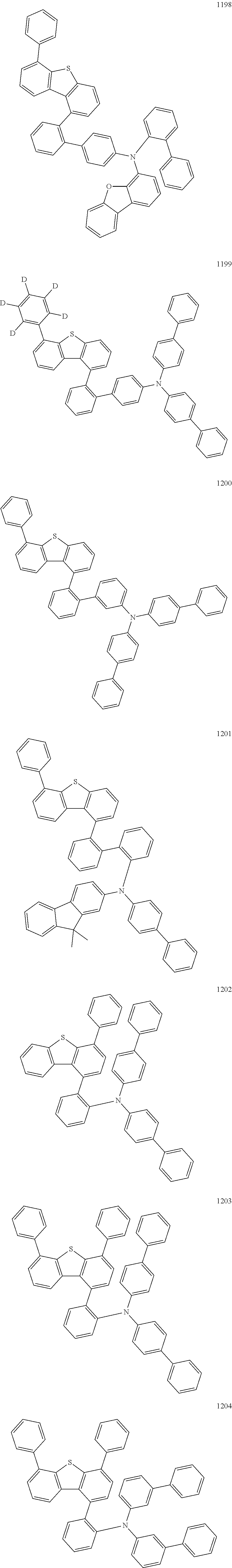

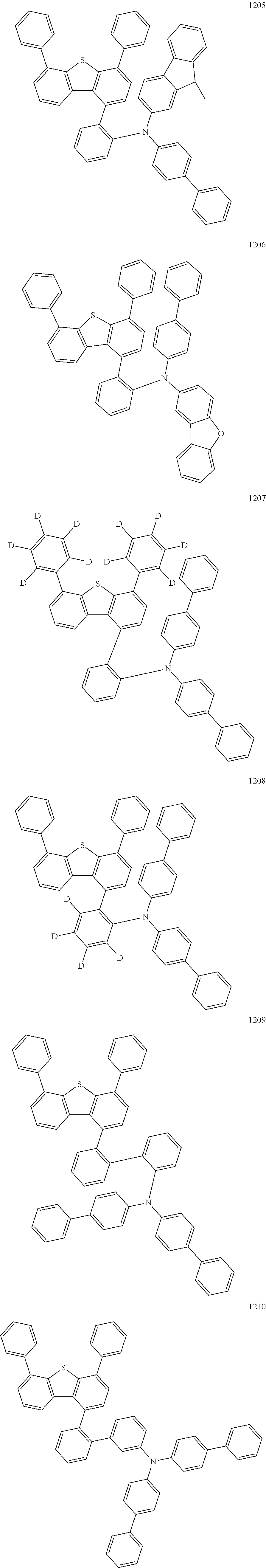

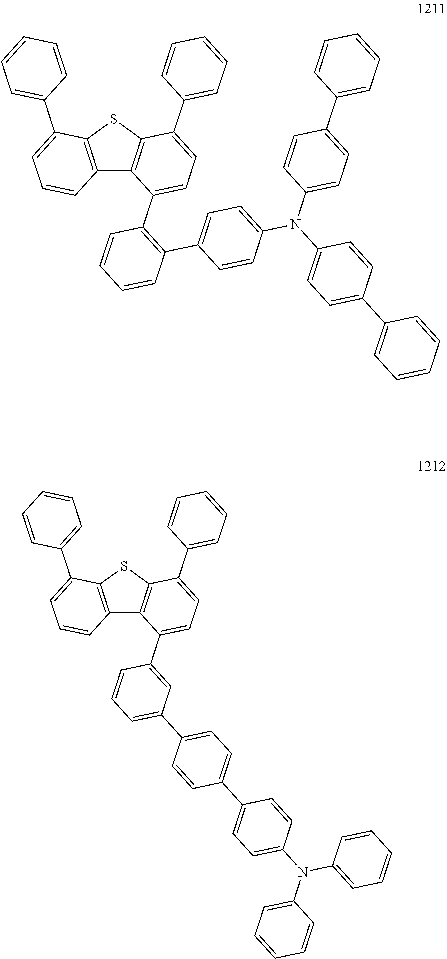 [SMILES: c1ccc(-c2ccc(N(c3ccc(-c4ccccc4)cc3)c3ccc(-c4ccccc4-c4ccc(-c5ccccc5)c5sc6c(-c7ccccc7)cccc6c45)cc3)cc2)cc1 ; c1ccc(-c2cccc3c2sc2c(-c4ccccc4)ccc(-c4cccc(-c5ccc(-c6ccc(N(c7ccccc7)c7ccccc7)cc6)cc5)c4)c23)cc1]

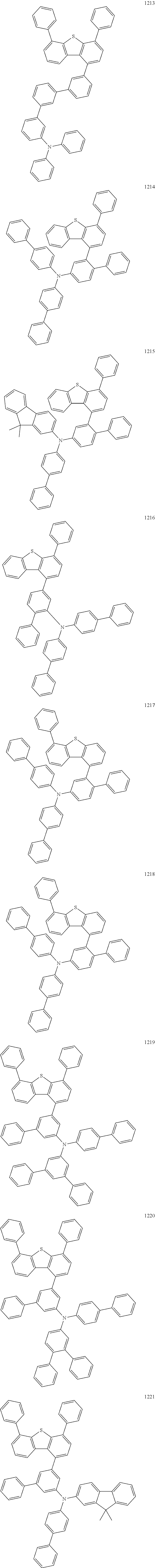

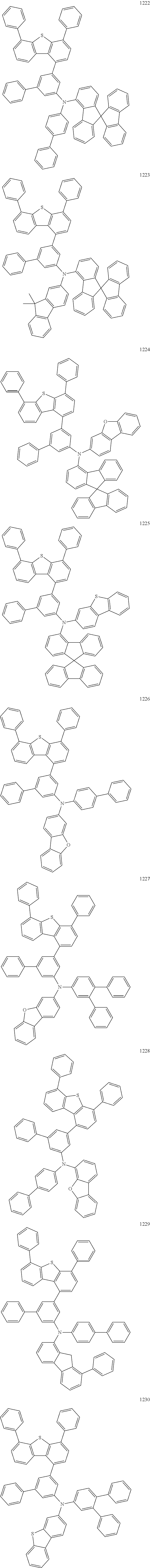

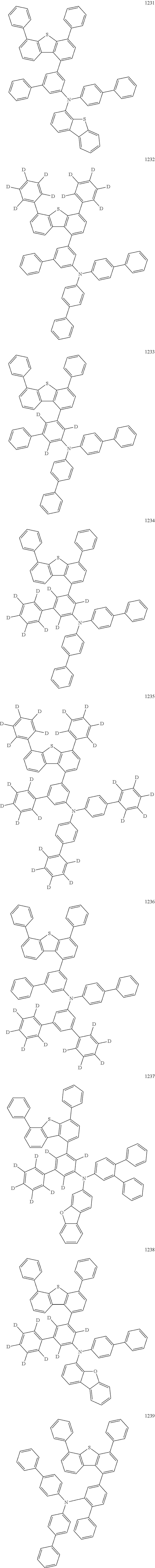

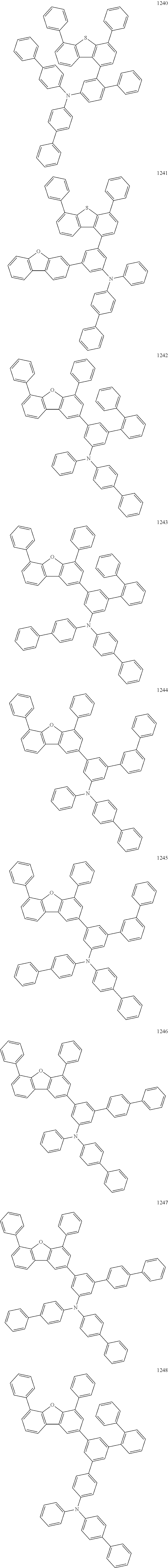

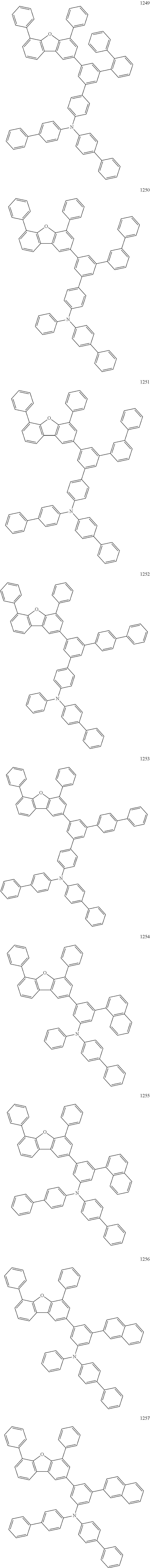

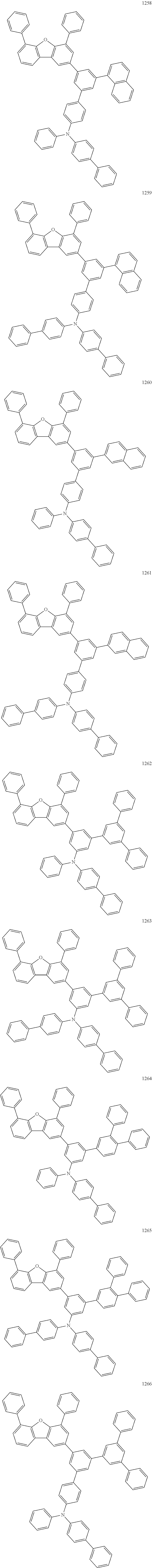

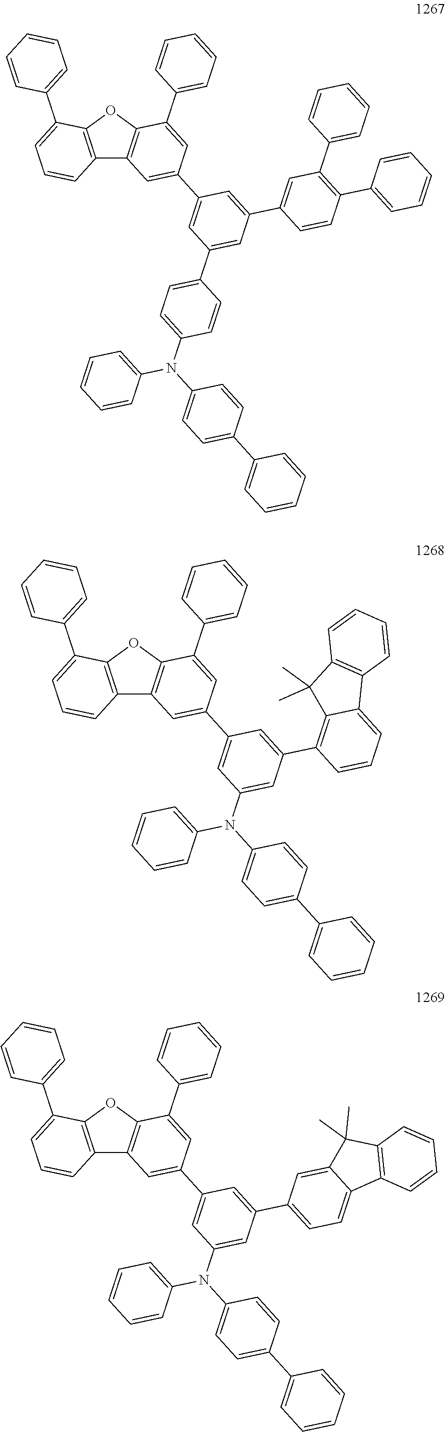 [SMILES: c1ccc(-c2ccc(N(c3ccccc3)c3ccc(-c4cc(-c5ccc(-c6ccccc6)c(-c6ccccc6)c5)cc(-c5cc(-c6ccccc6)c6oc7c(-c8ccccc8)cccc7c6c5)c4)cc3)cc2)cc1 ; CC1(C)c2ccccc2-c2cccc(-c3cc(-c4cc(-c5ccccc5)c5oc6c(-c7ccccc7)cccc6c5c4)cc(N(c4ccccc4)c4ccc(-c5ccccc5)cc4)c3)c21 ; CC1(C)c2ccccc2-c2ccc(-c3cc(-c4cc(-c5ccccc5)c5oc6c(-c7ccccc7)cccc6c5c4)cc(N(c4ccccc4)c4ccc(-c5ccccc5)cc4)c3)cc21]

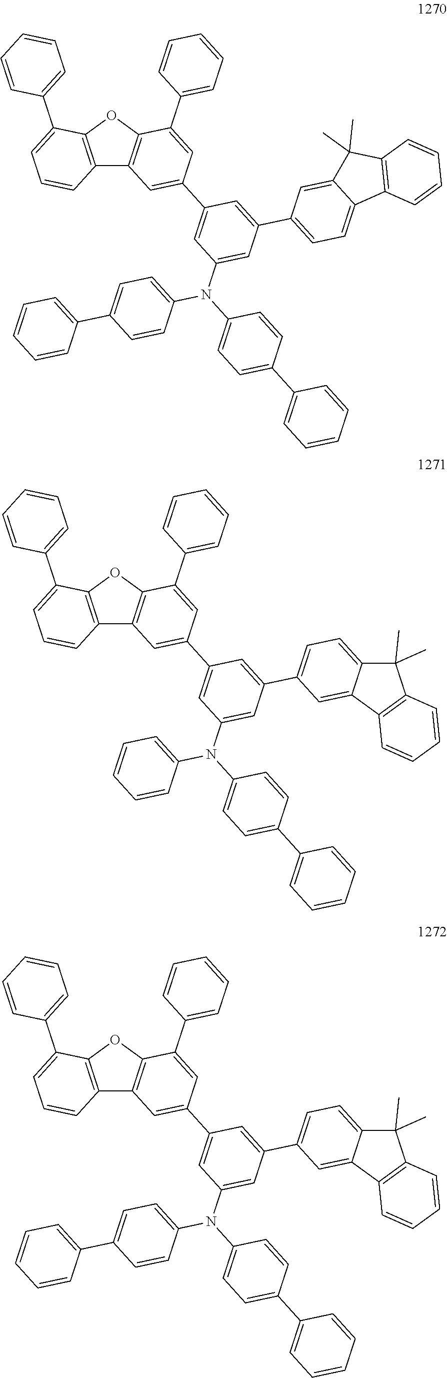 [SMILES: CC1(C)c2ccccc2-c2ccc(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)cc21 ; CC1(C)c2ccccc2-c2cc(-c3cc(-c4cc(-c5ccccc5)c5oc6c(-c7ccccc7)cccc6c5c4)cc(N(c4ccccc4)c4ccc(-c5ccccc5)cc4)c3)ccc21 ; CC1(C)c2ccccc2-c2cc(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)ccc21]

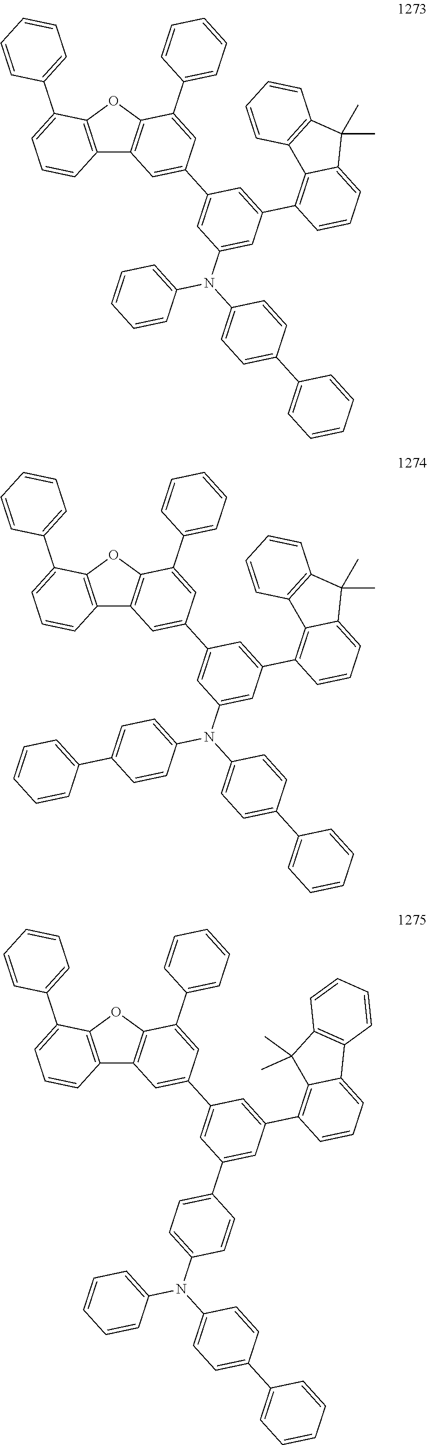 [SMILES: CC1(C)c2ccccc2-c2c(-c3cc(-c4cc(-c5ccccc5)c5oc6c(-c7ccccc7)cccc6c5c4)cc(N(c4ccccc4)c4ccc(-c5ccccc5)cc4)c3)cccc21 ; CC1(C)c2ccccc2-c2c(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)cccc21 ; CC1(C)c2ccccc2-c2cccc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)c21]

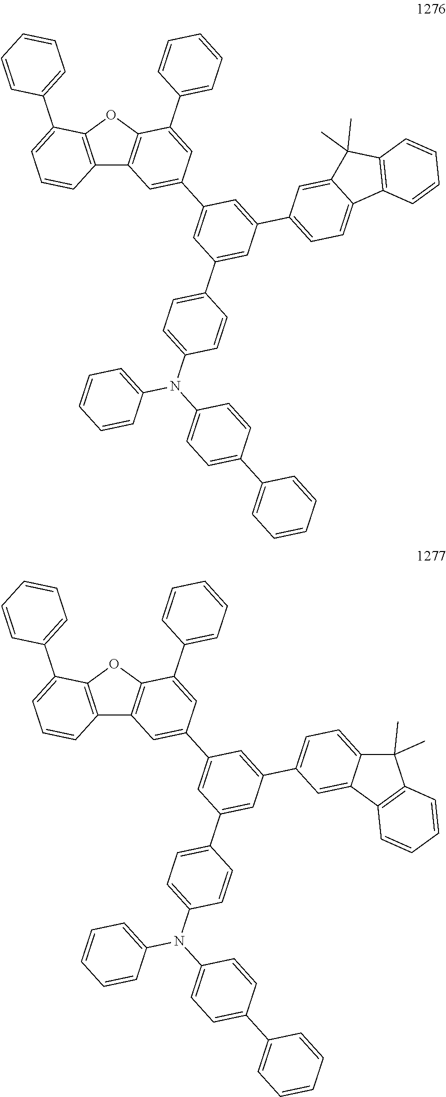 [SMILES: CC1(C)c2ccccc2-c2ccc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)cc21 ; CC1(C)c2ccccc2-c2cc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)ccc21]

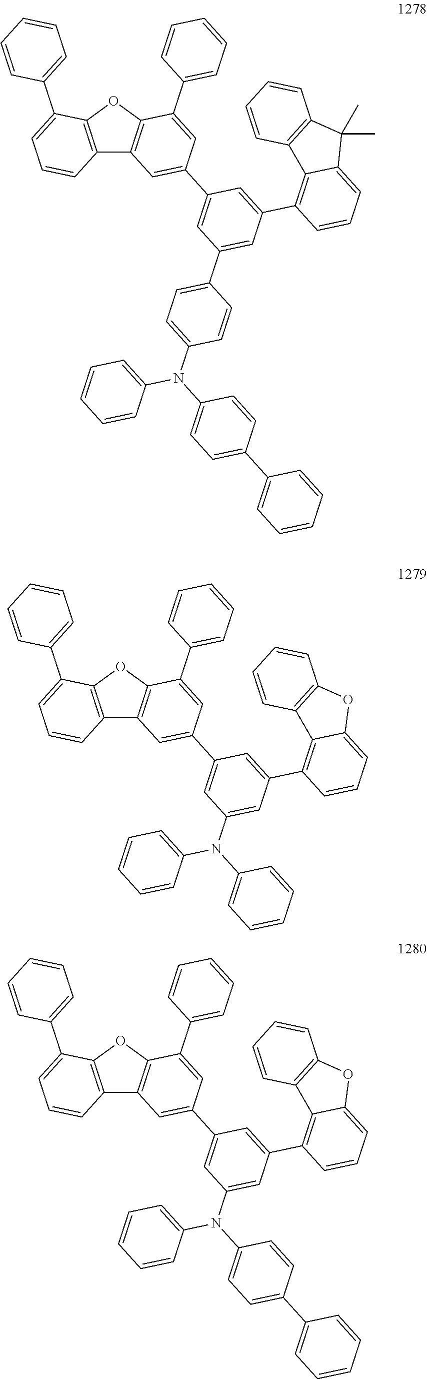 [SMILES: CC1(C)c2ccccc2-c2c(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)cccc21 ; c1ccc(-c2cccc3c2oc2c(-c4ccccc4)cc(-c4cc(-c5cccc6oc7ccccc7c56)cc(N(c5ccccc5)c5ccccc5)c4)cc23)cc1 ; c1ccc(-c2ccc(N(c3ccccc3)c3cc(-c4cc(-c5ccccc5)c5oc6c(-c7ccccc7)cccc6c5c4)cc(-c4cccc5oc6ccccc6c45)c3)cc2)cc1]

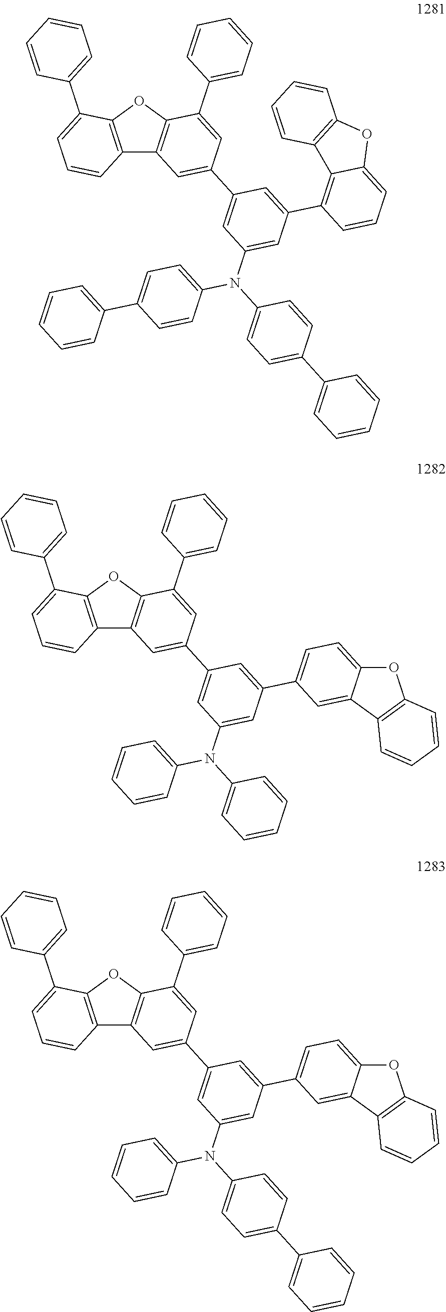 [SMILES: c1ccc(-c2ccc(N(c3ccc(-c4ccccc4)cc3)c3cc(-c4cc(-c5ccccc5)c5oc6c(-c7ccccc7)cccc6c5c4)cc(-c4cccc5oc6ccccc6c45)c3)cc2)cc1 ; c1ccc(-c2cccc3c2oc2c(-c4ccccc4)cc(-c4cc(-c5ccc6oc7ccccc7c6c5)cc(N(c5ccccc5)c5ccccc5)c4)cc23)cc1 ; c1ccc(-c2ccc(N(c3ccccc3)c3cc(-c4ccc5oc6ccccc6c5c4)cc(-c4cc(-c5ccccc5)c5oc6c(-c7ccccc7)cccc6c5c4)c3)cc2)cc1]

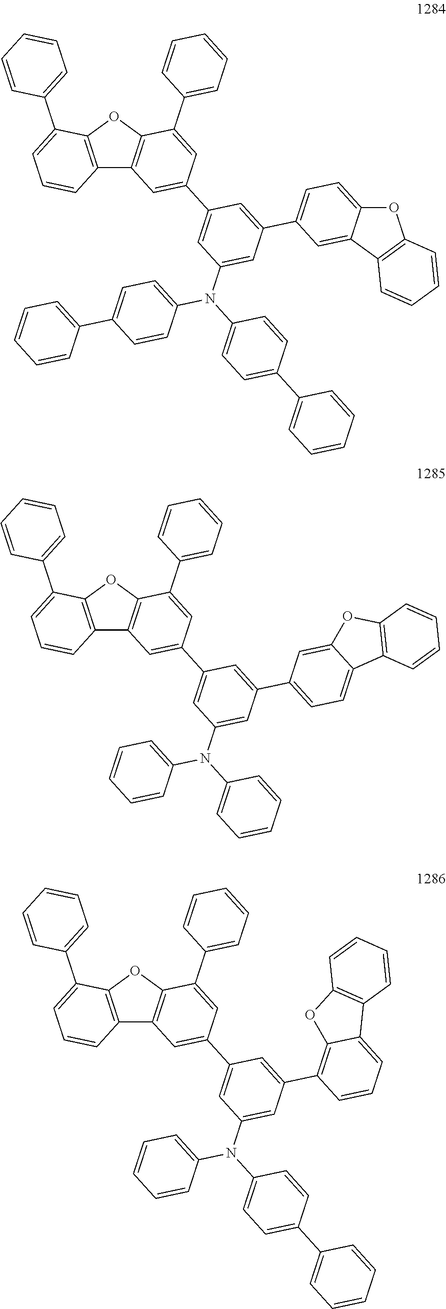 [SMILES: c1ccc(-c2ccc(N(c3ccc(-c4ccccc4)cc3)c3cc(-c4ccc5oc6ccccc6c5c4)cc(-c4cc(-c5ccccc5)c5oc6c(-c7ccccc7)cccc6c5c4)c3)cc2)cc1 ; c1ccc(-c2cccc3c2oc2c(-c4ccccc4)cc(-c4cc(-c5ccc6c(c5)oc5ccccc56)cc(N(c5ccccc5)c5ccccc5)c4)cc23)cc1 ; c1ccc(-c2ccc(N(c3ccccc3)c3cc(-c4cc(-c5ccccc5)c5oc6c(-c7ccccc7)cccc6c5c4)cc(-c4cccc5c4oc4ccccc45)c3)cc2)cc1]

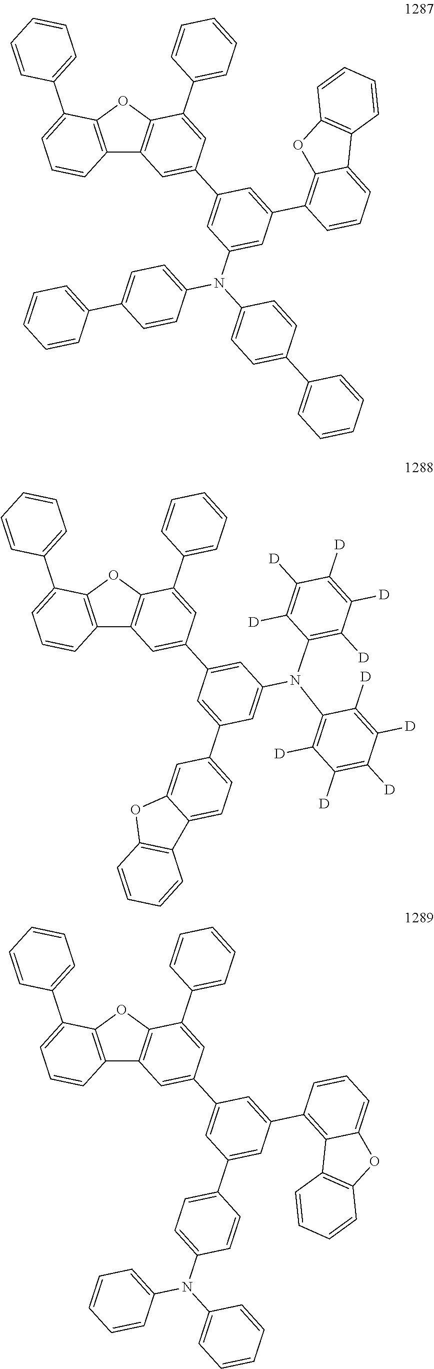

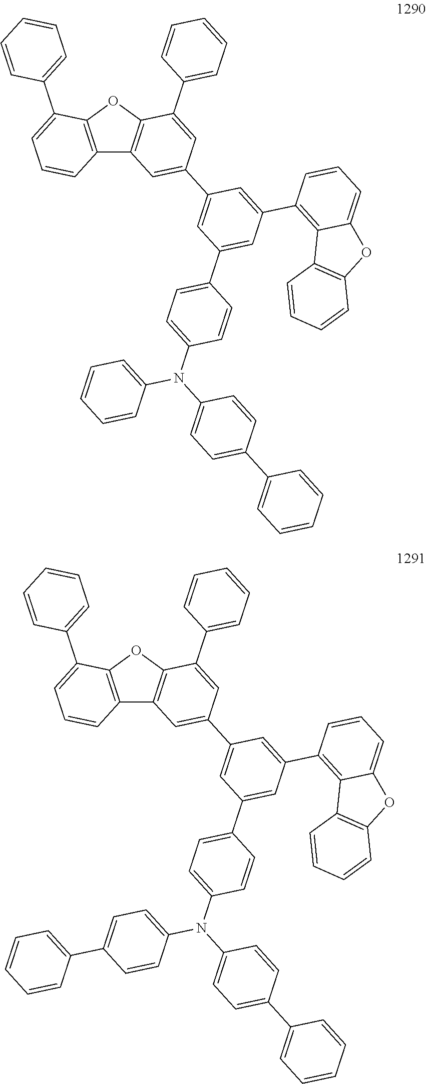 [SMILES: c1ccc(-c2ccc(N(c3ccccc3)c3ccc(-c4cc(-c5cc(-c6ccccc6)c6oc7c(-c8ccccc8)cccc7c6c5)cc(-c5cccc6oc7ccccc7c56)c4)cc3)cc2)cc1 ; c1ccc(-c2ccc(N(c3ccc(-c4ccccc4)cc3)c3ccc(-c4cc(-c5cc(-c6ccccc6)c6oc7c(-c8ccccc8)cccc7c6c5)cc(-c5cccc6oc7ccccc7c56)c4)cc3)cc2)cc1]

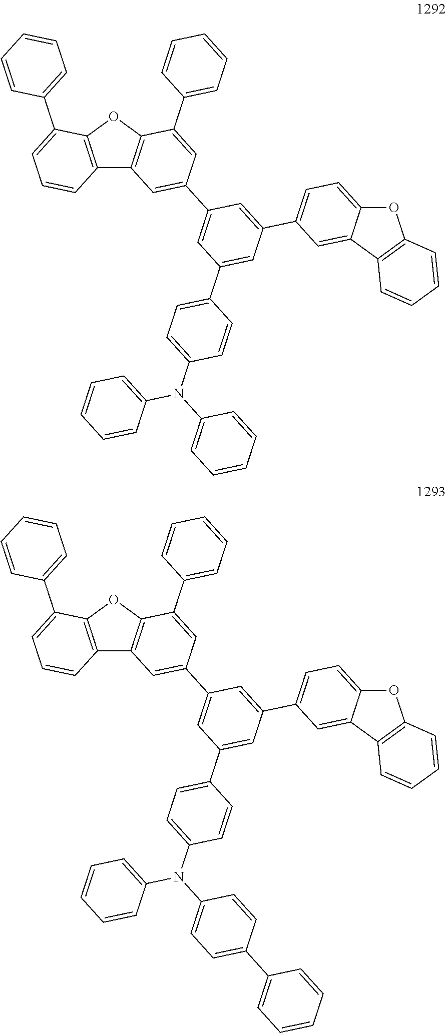

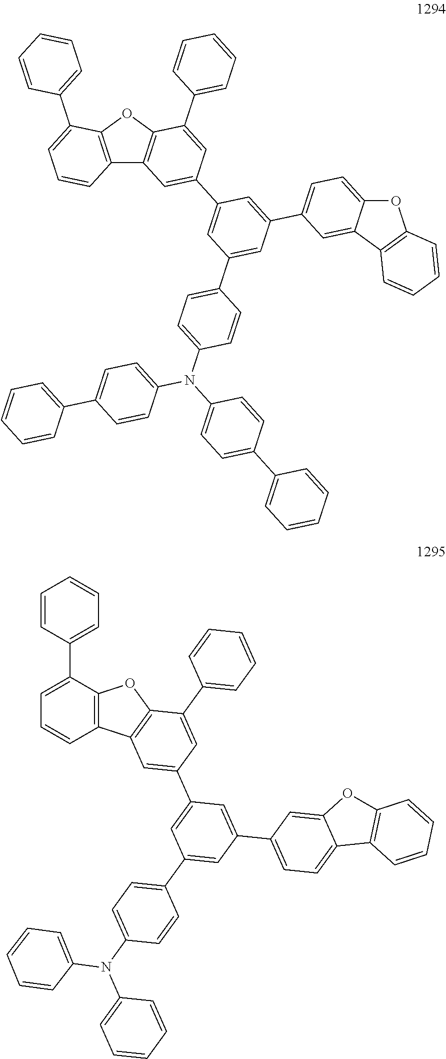 [SMILES: c1ccc(-c2ccc(N(c3ccc(-c4ccccc4)cc3)c3ccc(-c4cc(-c5ccc6oc7ccccc7c6c5)cc(-c5cc(-c6ccccc6)c6oc7c(-c8ccccc8)cccc7c6c5)c4)cc3)cc2)cc1 ; c1ccc(-c2cccc3c2oc2c(-c4ccccc4)cc(-c4cc(-c5ccc(N(c6ccccc6)c6ccccc6)cc5)cc(-c5ccc6c(c5)oc5ccccc56)c4)cc23)cc1]

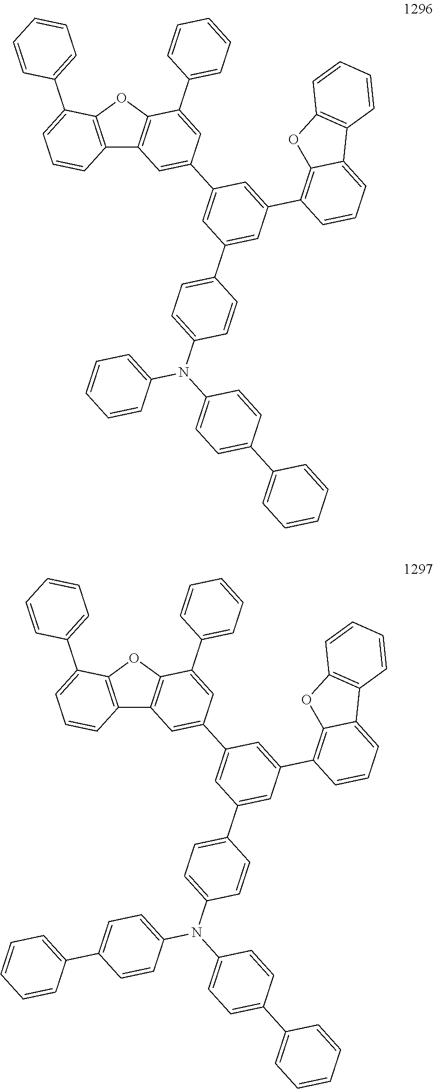 [SMILES: c1ccc(-c2ccc(N(c3ccccc3)c3ccc(-c4cc(-c5cc(-c6ccccc6)c6oc7c(-c8ccccc8)cccc7c6c5)cc(-c5cccc6c5oc5ccccc56)c4)cc3)cc2)cc1 ; c1ccc(-c2ccc(N(c3ccc(-c4ccccc4)cc3)c3ccc(-c4cc(-c5cc(-c6ccccc6)c6oc7c(-c8ccccc8)cccc7c6c5)cc(-c5cccc6c5oc5ccccc56)c4)cc3)cc2)cc1]

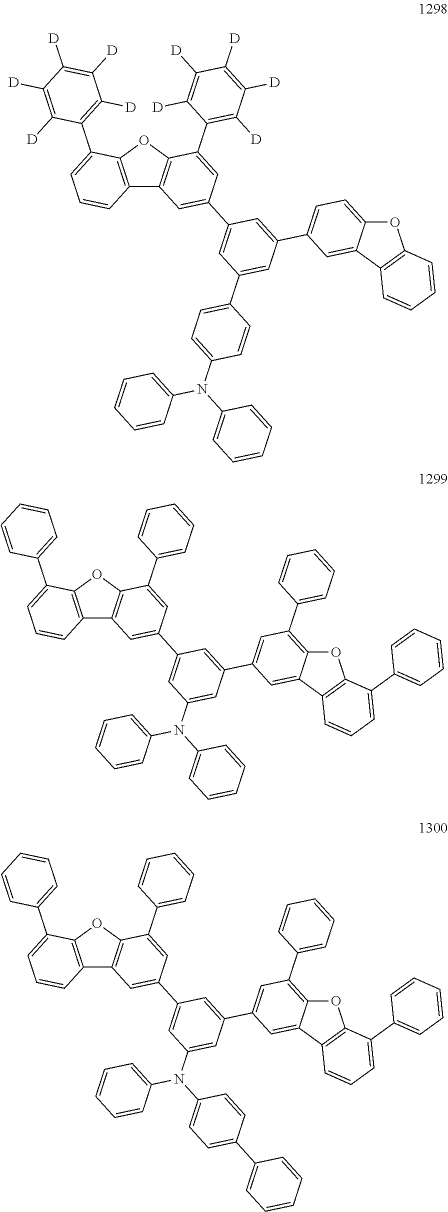 [SMILES: [2H]c1c([2H])c([2H])c(-c2cccc3c2oc2c(-c4c([2H])c([2H])c([2H])c([2H])c4[2H])cc(-c4cc(-c5ccc(N(c6ccccc6)c6ccccc6)cc5)cc(-c5ccc6oc7ccccc7c6c5)c4)cc23)c([2H])c1[2H] ; c1ccc(-c2cccc3c2oc2c(-c4ccccc4)cc(-c4cc(-c5cc(-c6ccccc6)c6oc7c(-c8ccccc8)cccc7c6c5)cc(N(c5ccccc5)c5ccccc5)c4)cc23)cc1 ; c1ccc(-c2ccc(N(c3ccccc3)c3cc(-c4cc(-c5ccccc5)c5oc6c(-c7ccccc7)cccc6c5c4)cc(-c4cc(-c5ccccc5)c5oc6c(-c7ccccc7)cccc6c5c4)c3)cc2)cc1]

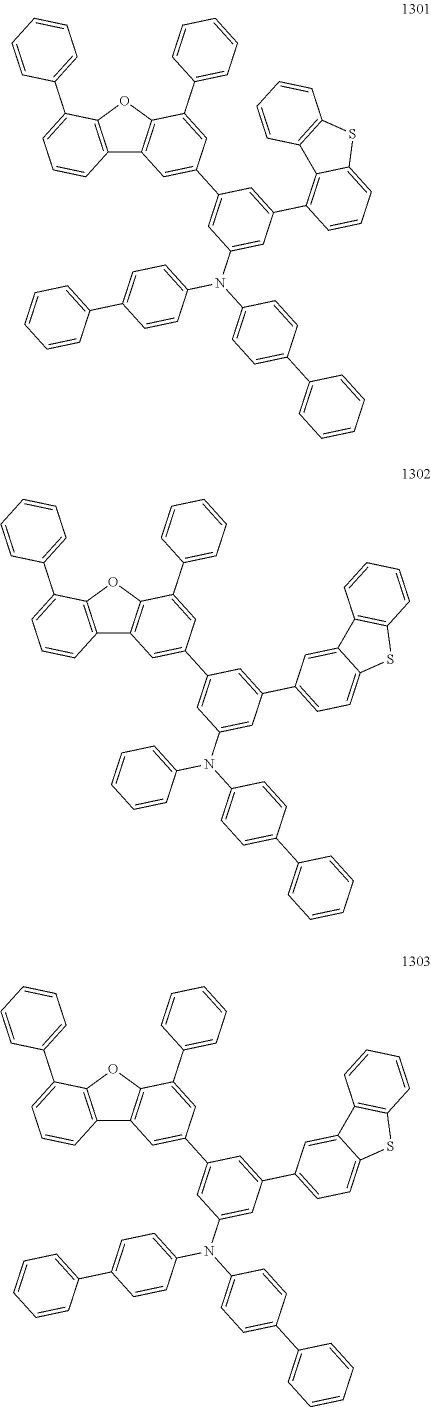 [SMILES: c1ccc(-c2ccc(N(c3ccc(-c4ccccc4)cc3)c3cc(-c4cc(-c5ccccc5)c5oc6c(-c7ccccc7)cccc6c5c4)cc(-c4cccc5sc6ccccc6c45)c3)cc2)cc1 ; c1ccc(-c2ccc(N(c3ccccc3)c3cc(-c4ccc5sc6ccccc6c5c4)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4ccc5sc6ccccc6c5c4)cc(-c4cc(-c5ccccc5)c5oc6c(-c7ccccc7)cccc6c5c4)c3)cc2)cc1]

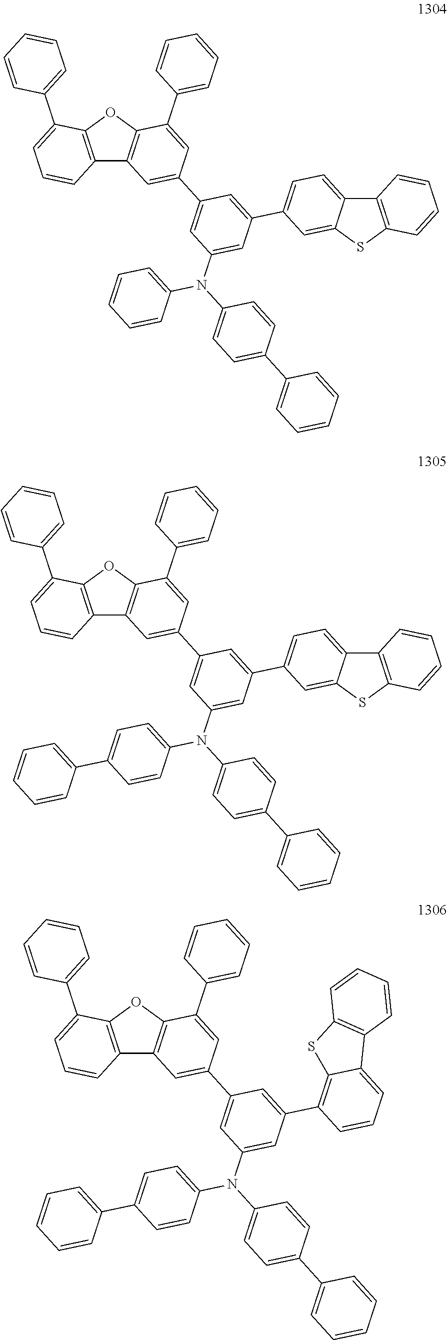 [SMILES: c1ccc(-c2ccc(N(c3ccccc3)c3cc(-c4ccc5c(c4)sc4ccccc45)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4ccc5c(c4)sc4ccccc45)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4cc(-c5ccccc5)c5oc6c(-c7ccccc7)cccc6c5c4)cc(-c4cccc5c4sc4ccccc45)c3)cc2)cc1]

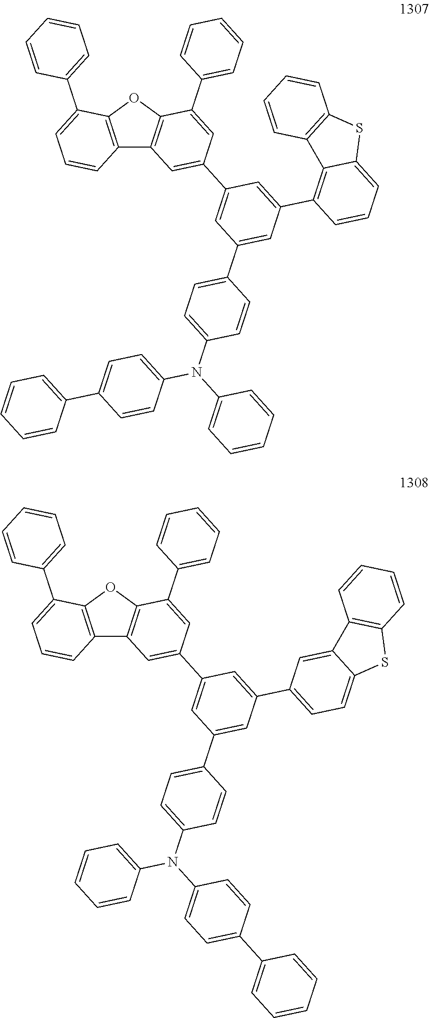 [SMILES: c1ccc(-c2ccc(N(c3ccccc3)c3ccc(-c4cc(-c5cc(-c6ccccc6)c6oc7c(-c8ccccc8)cccc7c6c5)cc(-c5cccc6sc7ccccc7c56)c4)cc3)cc2)cc1 ; c1ccc(-c2ccc(N(c3ccccc3)c3ccc(-c4cc(-c5ccc6sc7ccccc7c6c5)cc(-c5cc(-c6ccccc6)c6oc7c(-c8ccccc8)cccc7c6c5)c4)cc3)cc2)cc1]

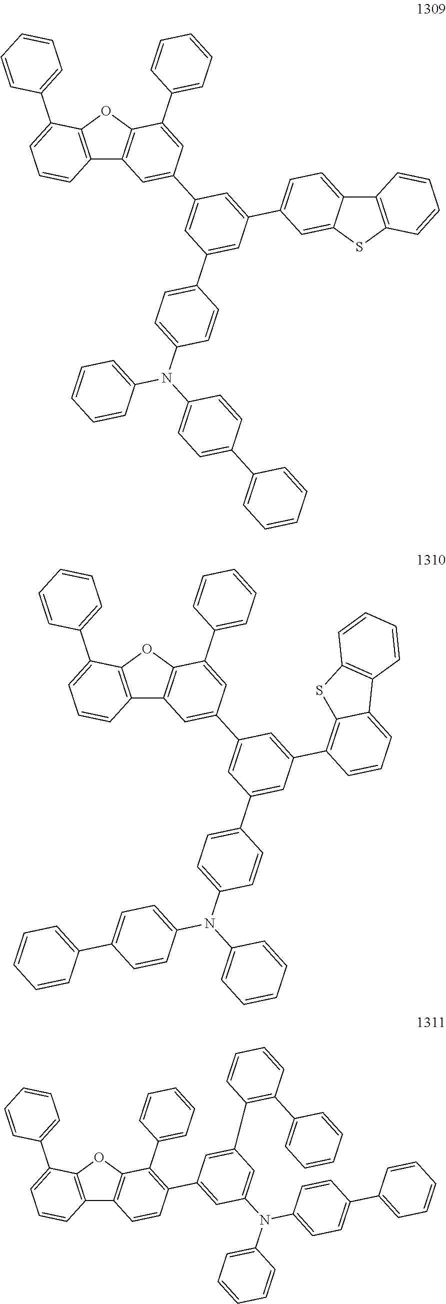 [SMILES: c1ccc(-c2ccc(N(c3ccccc3)c3ccc(-c4cc(-c5ccc6c(c5)sc5ccccc56)cc(-c5cc(-c6ccccc6)c6oc7c(-c8ccccc8)cccc7c6c5)c4)cc3)cc2)cc1 ; c1ccc(-c2ccc(N(c3ccccc3)c3ccc(-c4cc(-c5cc(-c6ccccc6)c6oc7c(-c8ccccc8)cccc7c6c5)cc(-c5cccc6c5sc5ccccc56)c4)cc3)cc2)cc1 ; c1ccc(-c2ccc(N(c3ccccc3)c3cc(-c4ccccc4-c4ccccc4)cc(-c4ccc5c(oc6c(-c7ccccc7)cccc65)c4-c4ccccc4)c3)cc2)cc1]

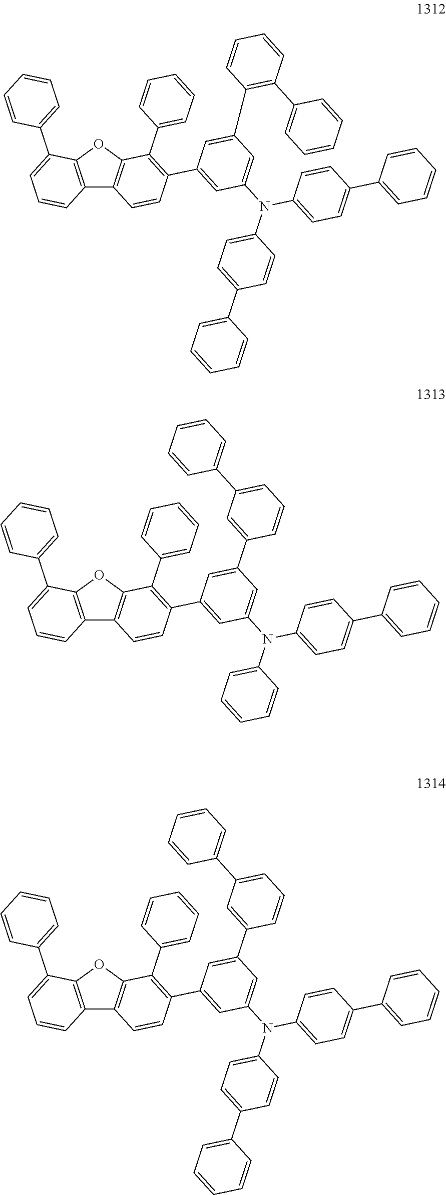 [SMILES: c1ccc(-c2ccc(N(c3ccc(-c4ccccc4)cc3)c3cc(-c4ccccc4-c4ccccc4)cc(-c4ccc5c(oc6c(-c7ccccc7)cccc65)c4-c4ccccc4)c3)cc2)cc1 ; c1ccc(-c2ccc(N(c3ccccc3)c3cc(-c4cccc(-c5ccccc5)c4)cc(-c4ccc5c(oc6c(-c7ccccc7)cccc65)c4-c4ccccc4)c3)cc2)cc1 ; c1ccc(-c2ccc(N(c3ccc(-c4ccccc4)cc3)c3cc(-c4cccc(-c5ccccc5)c4)cc(-c4ccc5c(oc6c(-c7ccccc7)cccc65)c4-c4ccccc4)c3)cc2)cc1]

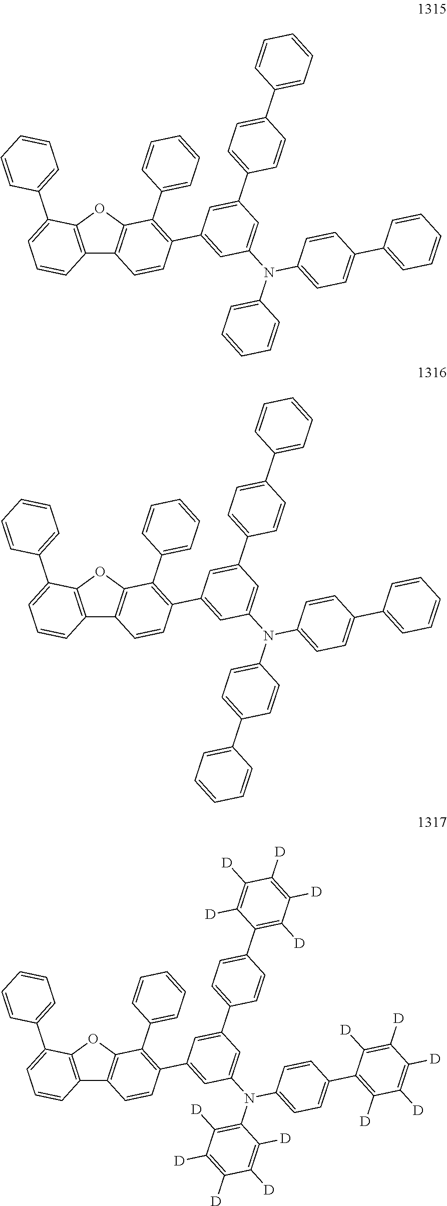 [SMILES: c1ccc(-c2ccc(-c3cc(-c4ccc5c(oc6c(-c7ccccc7)cccc65)c4-c4ccccc4)cc(N(c4ccccc4)c4ccc(-c5ccccc5)cc4)c3)cc2)cc1 ; c1ccc(-c2ccc(-c3cc(-c4ccc5c(oc6c(-c7ccccc7)cccc65)c4-c4ccccc4)cc(N(c4ccc(-c5ccccc5)cc4)c4ccc(-c5ccccc5)cc4)c3)cc2)cc1 ; [2H]c1c([2H])c([2H])c(-c2ccc(-c3cc(-c4ccc5c(oc6c(-c7ccccc7)cccc65)c4-c4ccccc4)cc(N(c4ccc(-c5c([2H])c([2H])c([2H])c([2H])c5[2H])cc4)c4c([2H])c([2H])c([2H])c([2H])c4[2H])c3)cc2)c([2H])c1[2H]]

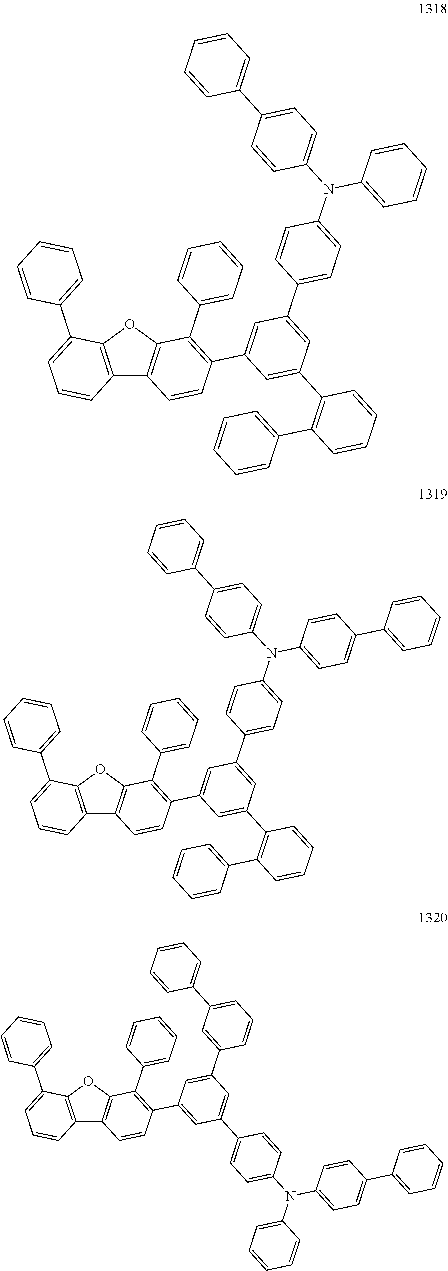 [SMILES: c1ccc(-c2ccc(N(c3ccccc3)c3ccc(-c4cc(-c5ccccc5-c5ccccc5)cc(-c5ccc6c(oc7c(-c8ccccc8)cccc76)c5-c5ccccc5)c4)cc3)cc2)cc1 ; c1ccc(-c2ccc(N(c3ccc(-c4ccccc4)cc3)c3ccc(-c4cc(-c5ccccc5-c5ccccc5)cc(-c5ccc6c(oc7c(-c8ccccc8)cccc76)c5-c5ccccc5)c4)cc3)cc2)cc1 ; c1ccc(-c2ccc(N(c3ccccc3)c3ccc(-c4cc(-c5cccc(-c6ccccc6)c5)cc(-c5ccc6c(oc7c(-c8ccccc8)cccc76)c5-c5ccccc5)c4)cc3)cc2)cc1]

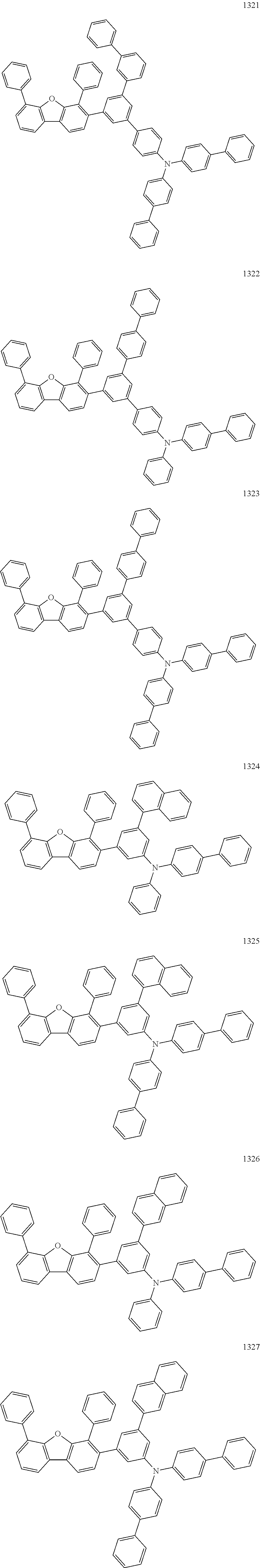

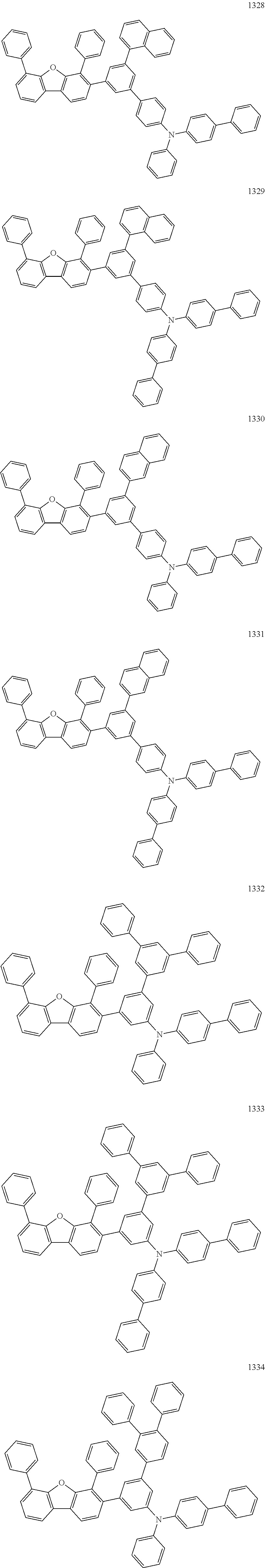

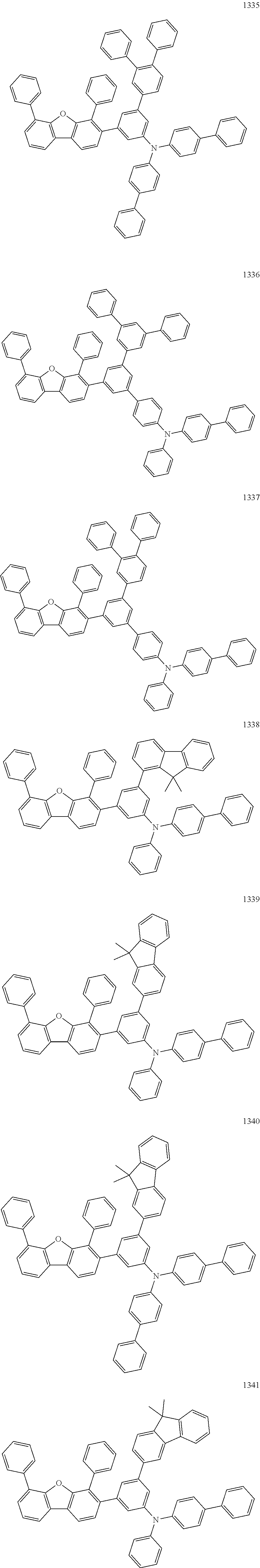

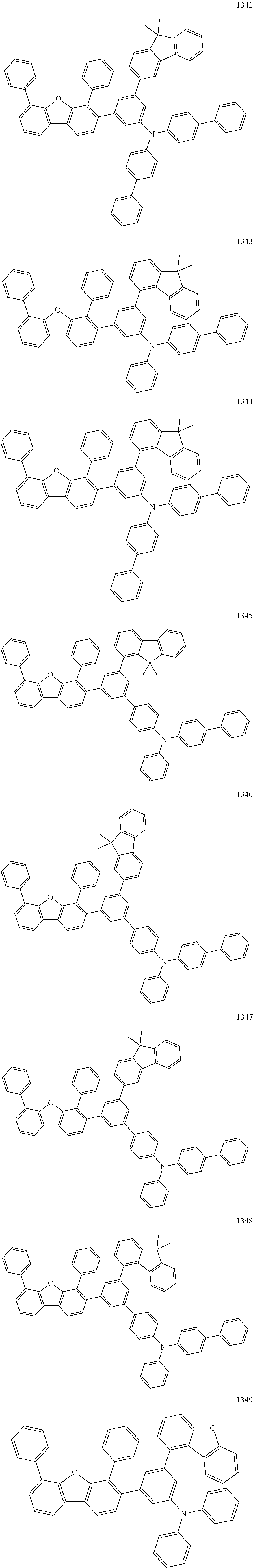

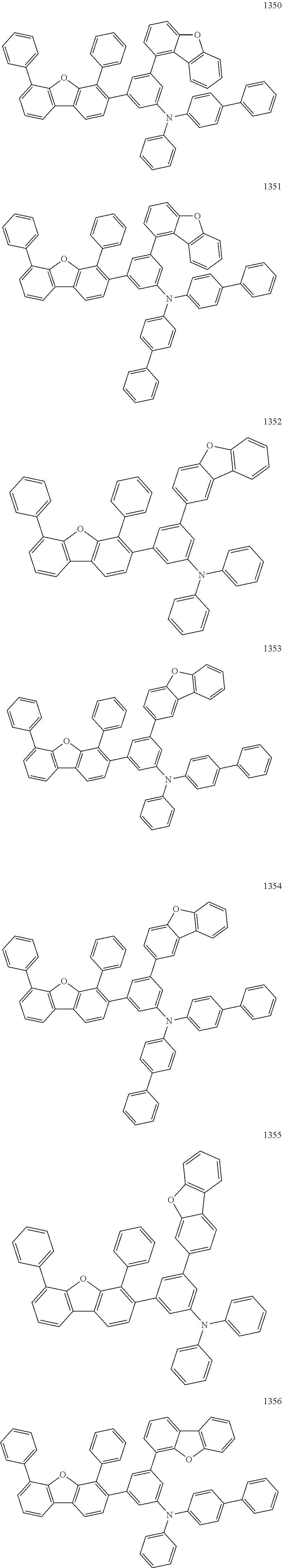

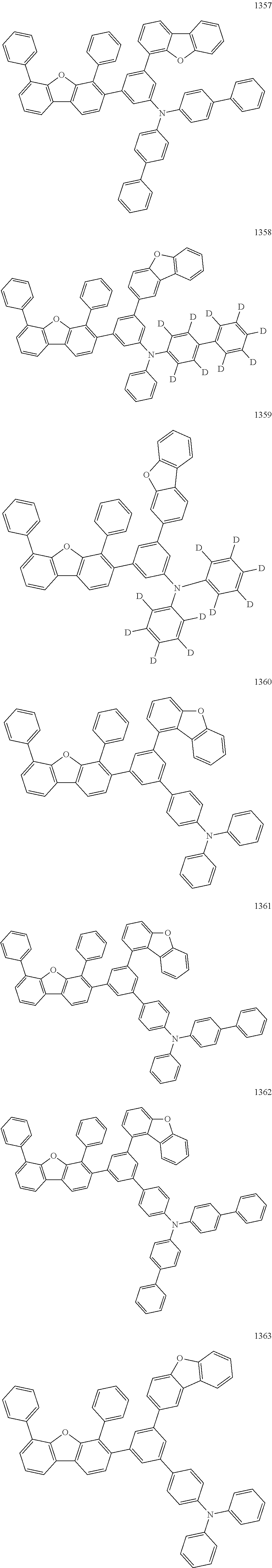

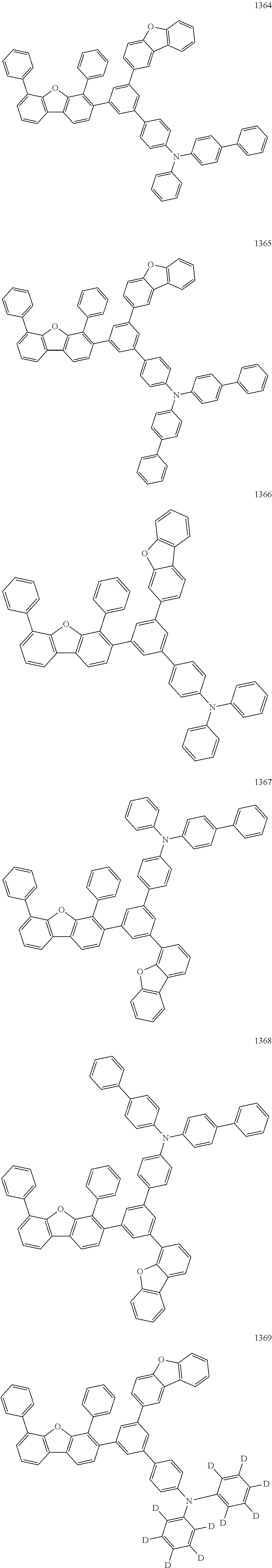

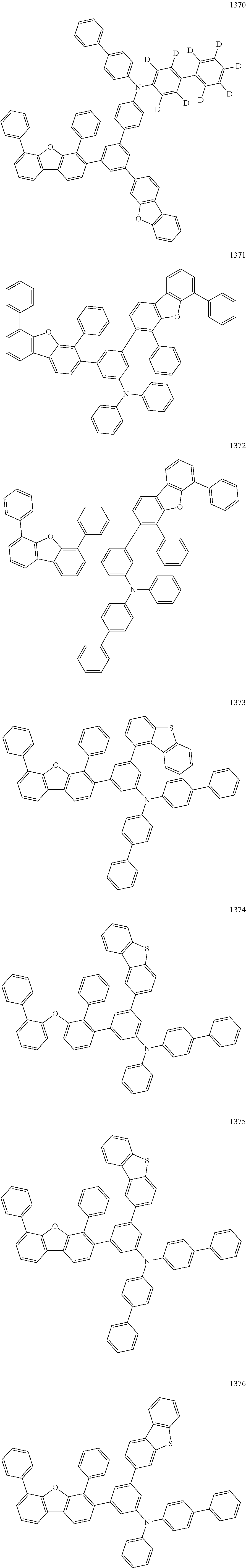

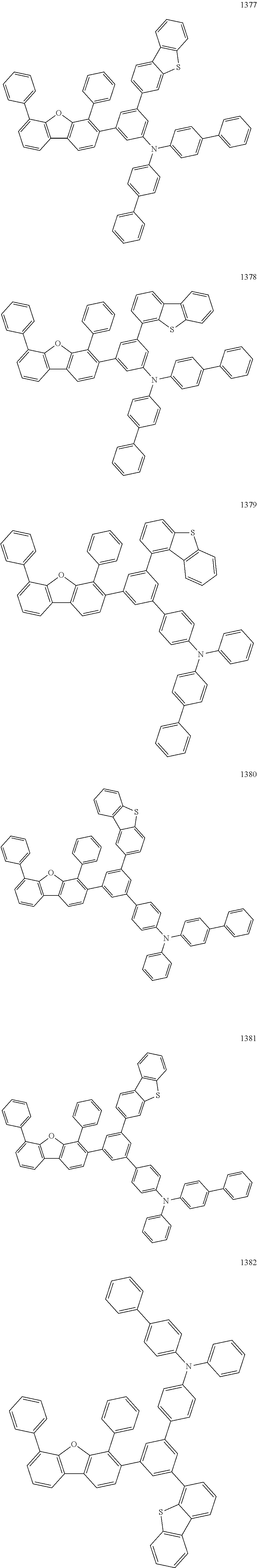

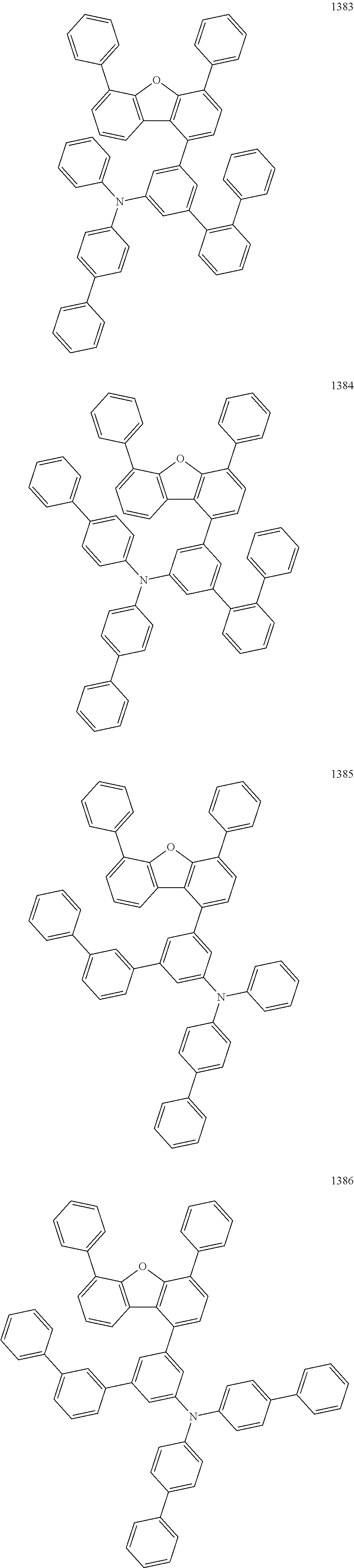 [SMILES: c1ccc(-c2ccc(N(c3ccccc3)c3cc(-c4ccccc4-c4ccccc4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4ccccc4-c4ccccc4)cc(-c4ccc(-c5ccccc5)c5oc6c(-c7ccccc7)cccc6c45)c3)cc2)cc1 ; c1ccc(-c2ccc(N(c3ccccc3)c3cc(-c4cccc(-c5ccccc5)c4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4cccc(-c5ccccc5)c4)cc(-c4ccc(-c5ccccc5)c5oc6c(-c7ccccc7)cccc6c45)c3)cc2)cc1]

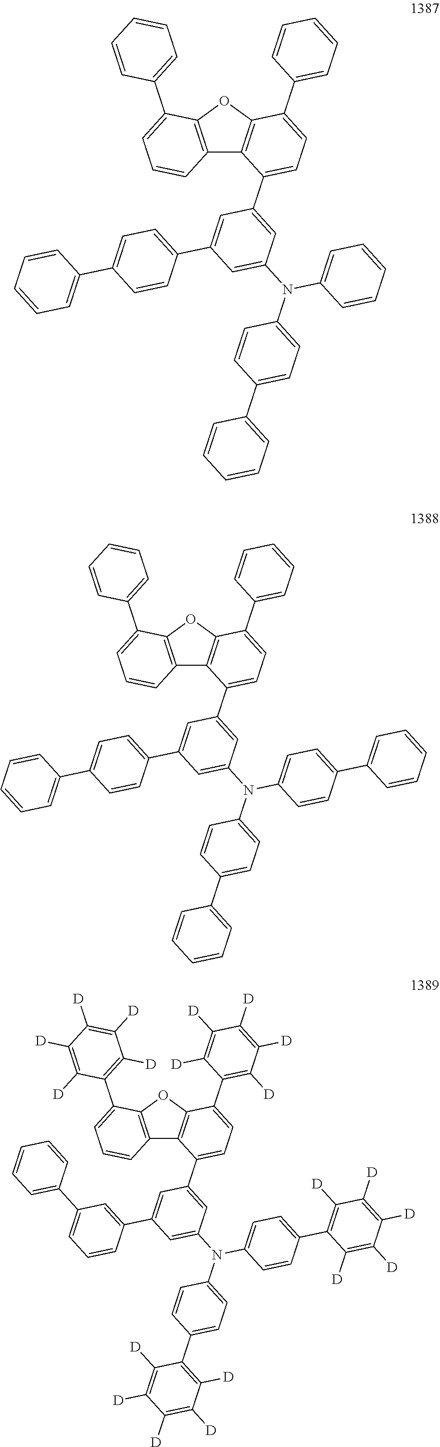 [SMILES: c1ccc(-c2ccc(-c3cc(-c4ccc(-c5ccccc5)c5oc6c(-c7ccccc7)cccc6c45)cc(N(c4ccccc4)c4ccc(-c5ccccc5)cc4)c3)cc2)cc1 ; c1ccc(-c2ccc(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)cc2)cc1 ; [2H]c1c([2H])c([2H])c(-c2ccc(N(c3ccc(-c4c([2H])c([2H])c([2H])c([2H])c4[2H])cc3)c3cc(-c4cccc(-c5ccccc5)c4)cc(-c4ccc(-c5c([2H])c([2H])c([2H])c([2H])c5[2H])c5oc6c(-c7c([2H])c([2H])c([2H])c([2H])c7[2H])cccc6c45)c3)cc2)c([2H])c1[2H]]

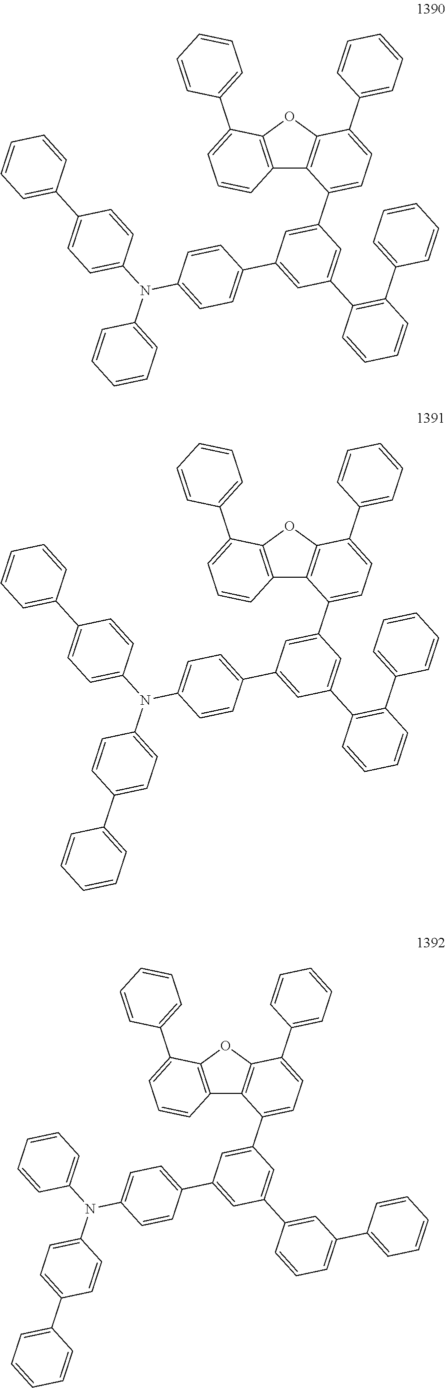 [SMILES: c1ccc(-c2ccc(N(c3ccccc3)c3ccc(-c4cc(-c5ccccc5-c5ccccc5)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5ccccc5-c5ccccc5)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5cccc(-c6ccccc6)c5)cc(-c5ccc(-c6ccccc6)c6oc7c(-c8ccccc8)cccc7c56)c4)cc3)cc2)cc1]

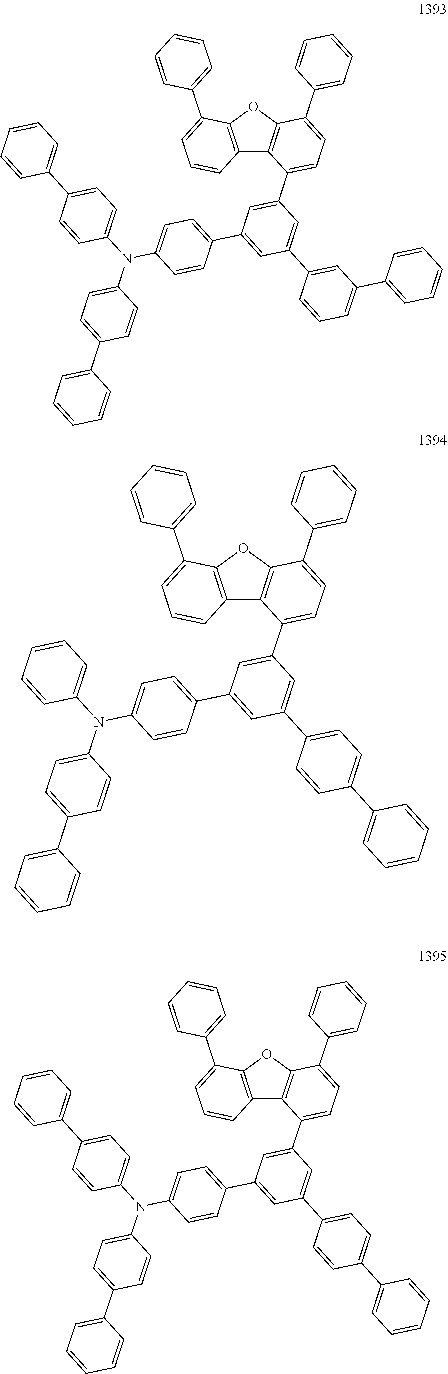 [SMILES: c1ccc(-c2ccc(N(c3ccc(-c4ccccc4)cc3)c3ccc(-c4cc(-c5cccc(-c6ccccc6)c5)cc(-c5ccc(-c6ccccc6)c6oc7c(-c8ccccc8)cccc7c56)c4)cc3)cc2)cc1 ; c1ccc(-c2ccc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)cc2)cc1 ; c1ccc(-c2ccc(-c3cc(-c4ccc(N(c5ccc(-c6ccccc6)cc5)c5ccc(-c6ccccc6)cc5)cc4)cc(-c4ccc(-c5ccccc5)c5oc6c(-c7ccccc7)cccc6c45)c3)cc2)cc1]

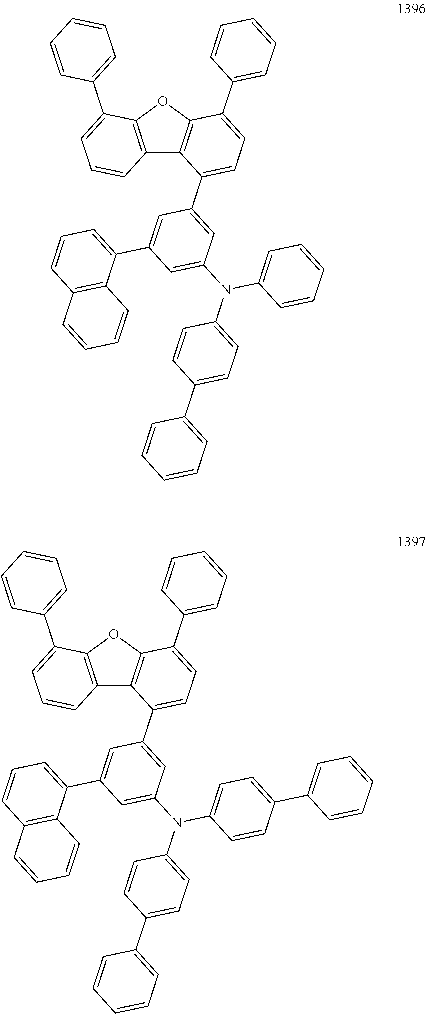 [SMILES: c1ccc(-c2ccc(N(c3ccccc3)c3cc(-c4cccc5ccccc45)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4cccc5ccccc45)cc(-c4ccc(-c5ccccc5)c5oc6c(-c7ccccc7)cccc6c45)c3)cc2)cc1]

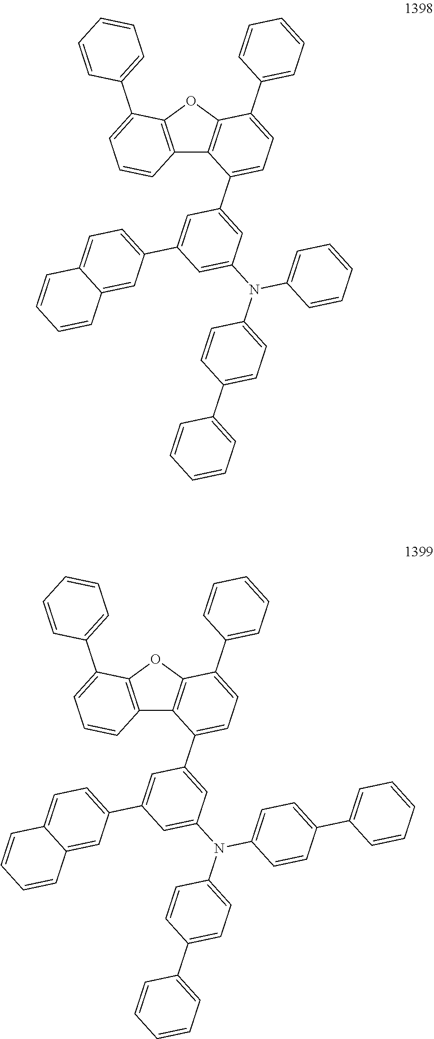 [SMILES: c1ccc(-c2ccc(N(c3ccccc3)c3cc(-c4ccc5ccccc5c4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4ccc5ccccc5c4)cc(-c4ccc(-c5ccccc5)c5oc6c(-c7ccccc7)cccc6c45)c3)cc2)cc1]

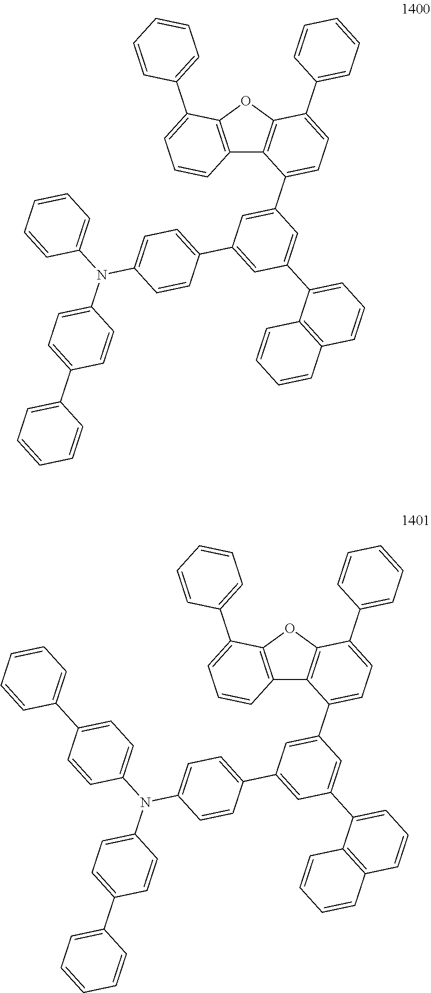 [SMILES: c1ccc(-c2ccc(N(c3ccccc3)c3ccc(-c4cc(-c5cccc6ccccc56)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5cccc6ccccc56)cc(-c5ccc(-c6ccccc6)c6oc7c(-c8ccccc8)cccc7c56)c4)cc3)cc2)cc1]

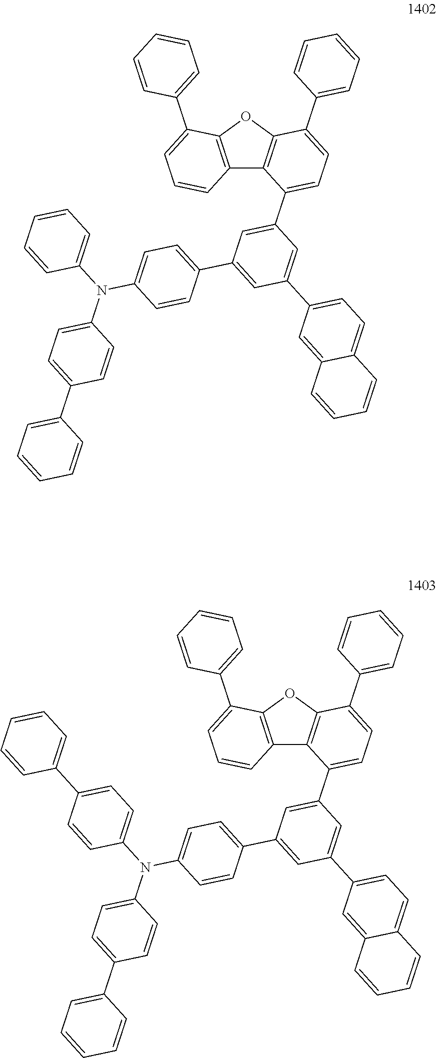 [SMILES: c1ccc(-c2ccc(N(c3ccccc3)c3ccc(-c4cc(-c5ccc6ccccc6c5)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5ccc6ccccc6c5)cc(-c5ccc(-c6ccccc6)c6oc7c(-c8ccccc8)cccc7c56)c4)cc3)cc2)cc1]

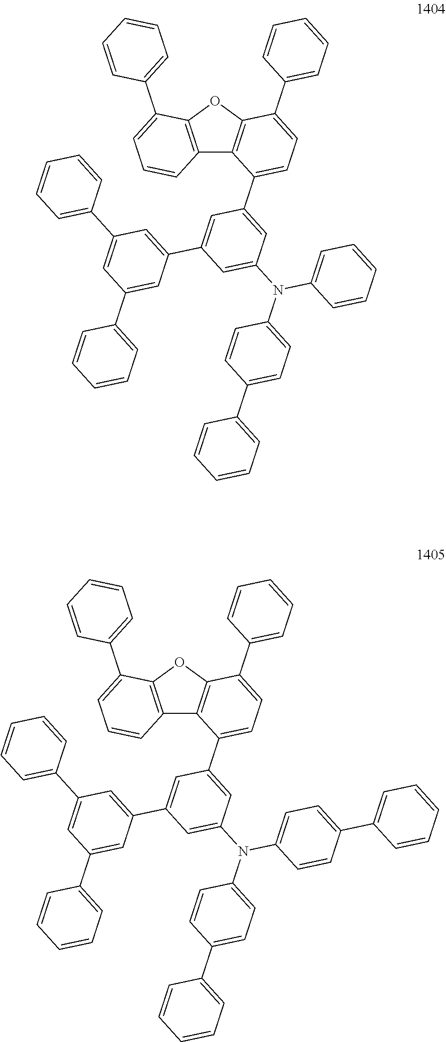 [SMILES: c1ccc(-c2ccc(N(c3ccccc3)c3cc(-c4cc(-c5ccccc5)cc(-c5ccccc5)c4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4cc(-c5ccccc5)cc(-c5ccccc5)c4)cc(-c4ccc(-c5ccccc5)c5oc6c(-c7ccccc7)cccc6c45)c3)cc2)cc1]

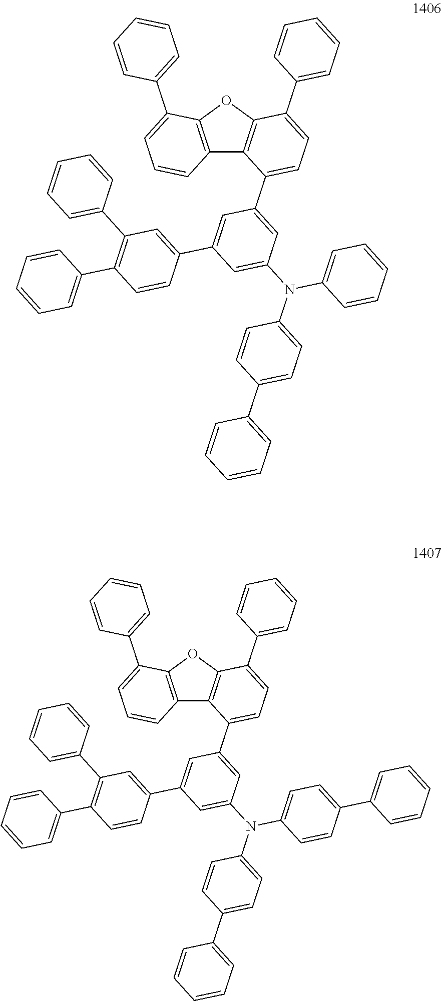 [SMILES: c1ccc(-c2ccc(N(c3ccccc3)c3cc(-c4ccc(-c5ccccc5)c(-c5ccccc5)c4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4ccc(-c5ccccc5)c(-c5ccccc5)c4)cc(-c4ccc(-c5ccccc5)c5oc6c(-c7ccccc7)cccc6c45)c3)cc2)cc1]

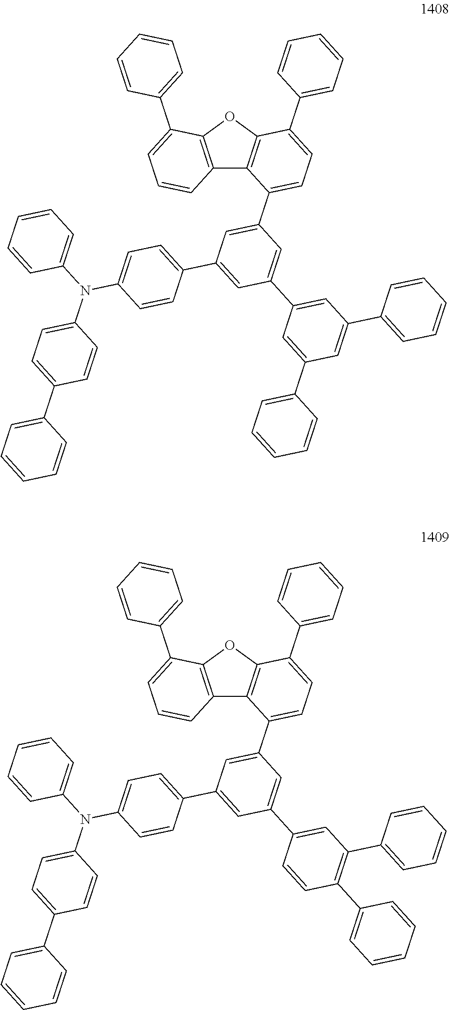 [SMILES: c1ccc(-c2ccc(N(c3ccccc3)c3ccc(-c4cc(-c5cc(-c6ccccc6)cc(-c6ccccc6)c5)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5ccc(-c6ccccc6)c(-c6ccccc6)c5)cc(-c5ccc(-c6ccccc6)c6oc7c(-c8ccccc8)cccc7c56)c4)cc3)cc2)cc1]

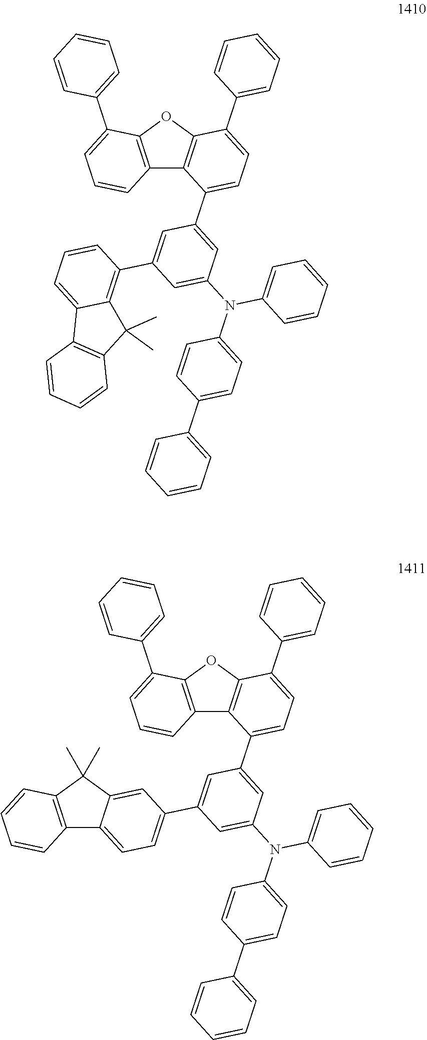 [SMILES: CC1(C)c2ccccc2-c2cccc(-c3cc(-c4ccc(-c5ccccc5)c5oc6c(-c7ccccc7)cccc6c45)cc(N(c4ccccc4)c4ccc(-c5ccccc5)cc4)c3)c21 ; CC1(C)c2ccccc2-c2ccc(-c3cc(-c4ccc(-c5ccccc5)c5oc6c(-c7ccccc7)cccc6c45)cc(N(c4ccccc4)c4ccc(-c5ccccc5)cc4)c3)cc21]

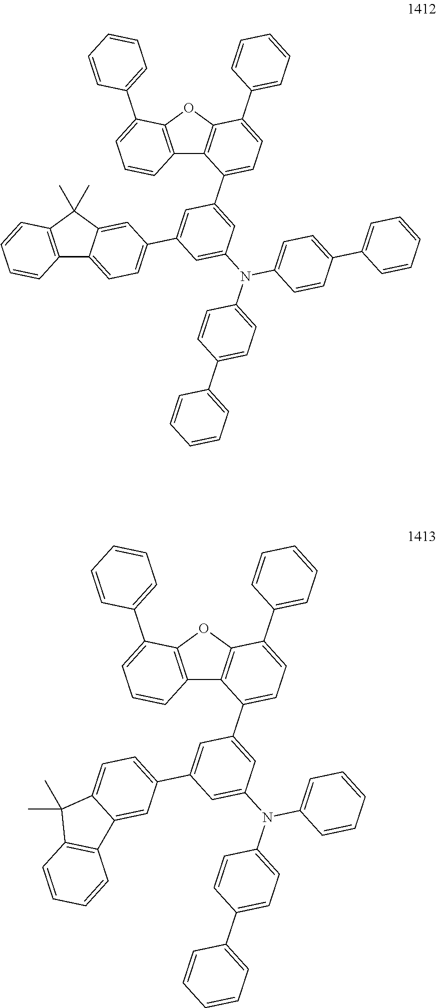 [SMILES: CC1(C)c2ccccc2-c2ccc(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)cc21 ; CC1(C)c2ccccc2-c2cc(-c3cc(-c4ccc(-c5ccccc5)c5oc6c(-c7ccccc7)cccc6c45)cc(N(c4ccccc4)c4ccc(-c5ccccc5)cc4)c3)ccc21]

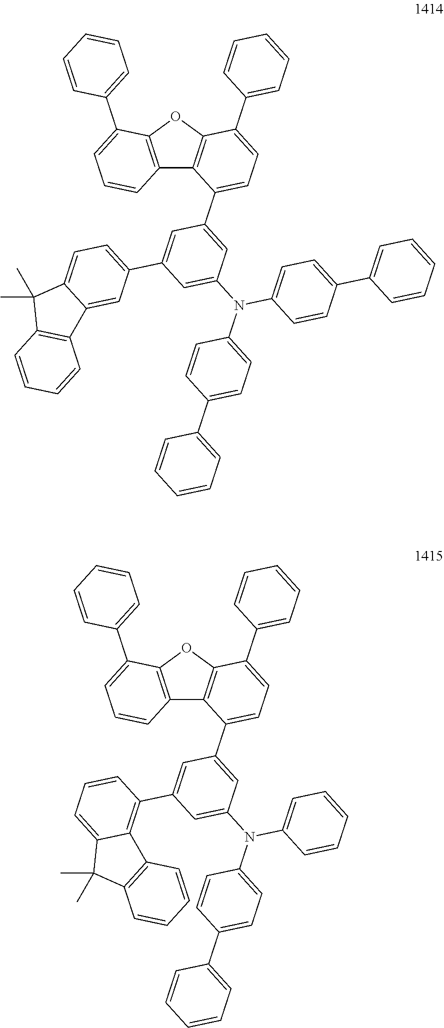 [SMILES: CC1(C)c2ccccc2-c2cc(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)ccc21 ; CC1(C)c2ccccc2-c2c(-c3cc(-c4ccc(-c5ccccc5)c5oc6c(-c7ccccc7)cccc6c45)cc(N(c4ccccc4)c4ccc(-c5ccccc5)cc4)c3)cccc21]

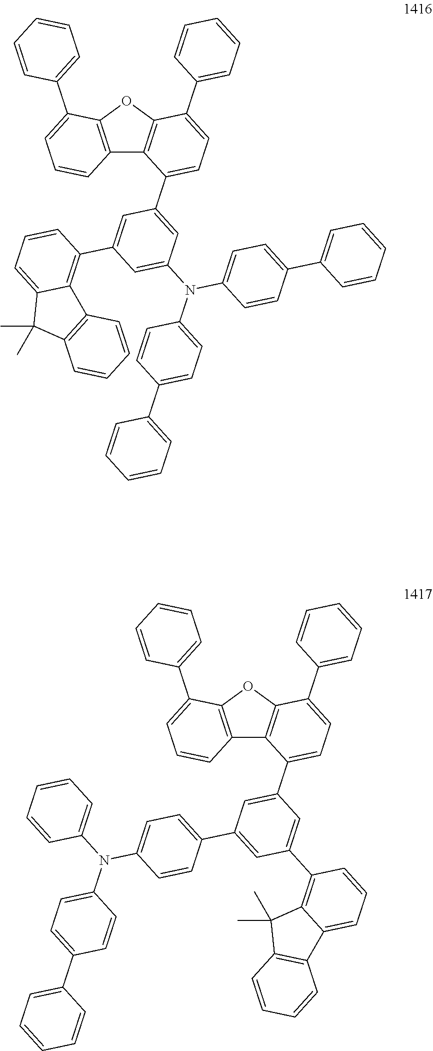 [SMILES: CC1(C)c2ccccc2-c2c(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)cccc21 ; CC1(C)c2ccccc2-c2cccc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)c21]

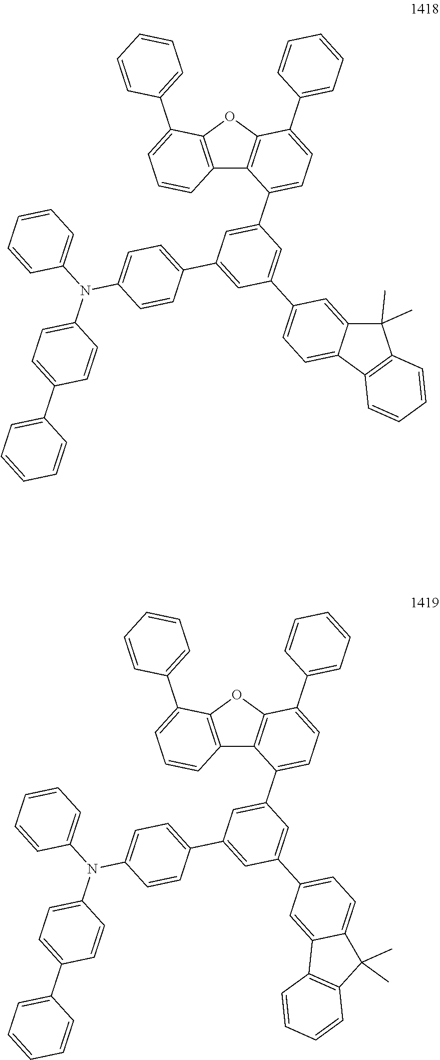 [SMILES: CC1(C)c2ccccc2-c2ccc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)cc21 ; CC1(C)c2ccccc2-c2cc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)ccc21]

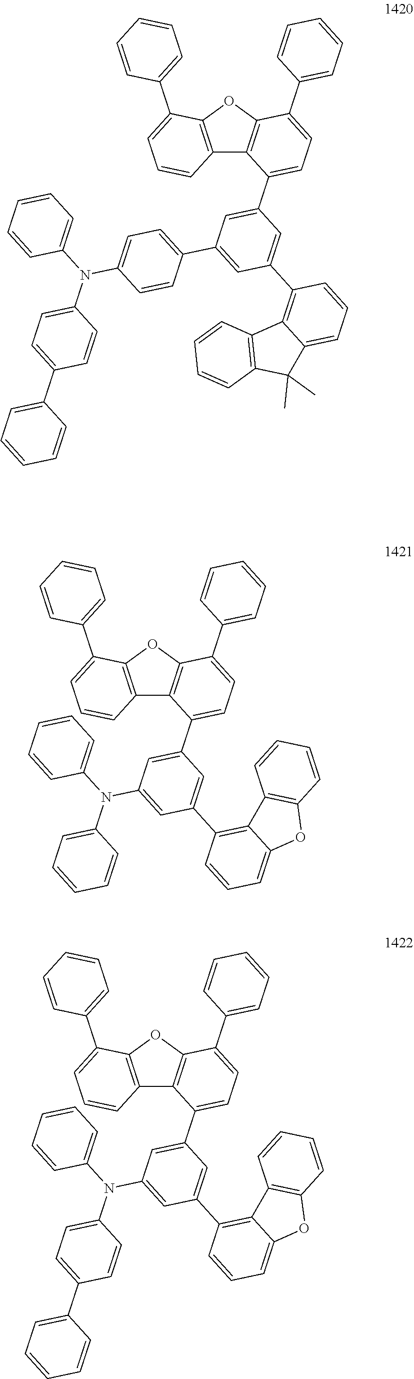 [SMILES: CC1(C)c2ccccc2-c2c(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)cccc21 ; c1ccc(-c2cccc3c2oc2c(-c4ccccc4)ccc(-c4cc(-c5cccc6oc7ccccc7c56)cc(N(c5ccccc5)c5ccccc5)c4)c23)cc1 ; c1ccc(-c2ccc(N(c3ccccc3)c3cc(-c4cccc5oc6ccccc6c45)cc(-c4ccc(-c5ccccc5)c5oc6c(-c7ccccc7)cccc6c45)c3)cc2)cc1]

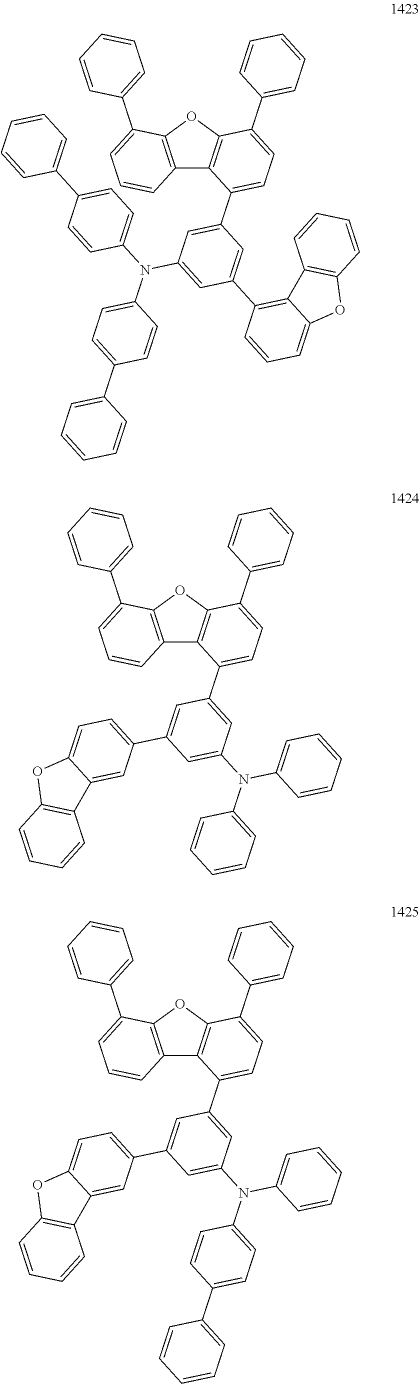 [SMILES: c1ccc(-c2ccc(N(c3ccc(-c4ccccc4)cc3)c3cc(-c4cccc5oc6ccccc6c45)cc(-c4ccc(-c5ccccc5)c5oc6c(-c7ccccc7)cccc6c45)c3)cc2)cc1 ; c1ccc(-c2cccc3c2oc2c(-c4ccccc4)ccc(-c4cc(-c5ccc6oc7ccccc7c6c5)cc(N(c5ccccc5)c5ccccc5)c4)c23)cc1 ; c1ccc(-c2ccc(N(c3ccccc3)c3cc(-c4ccc5oc6ccccc6c5c4)cc(-c4ccc(-c5ccccc5)c5oc6c(-c7ccccc7)cccc6c45)c3)cc2)cc1]

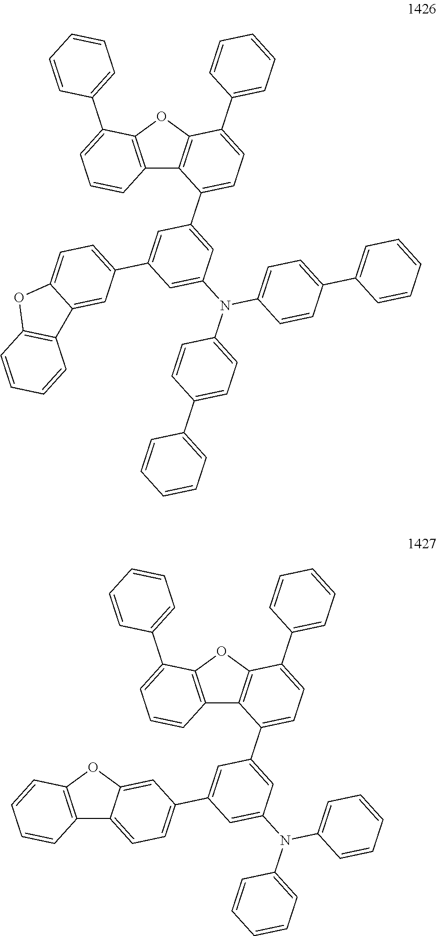 [SMILES: c1ccc(-c2ccc(N(c3ccc(-c4ccccc4)cc3)c3cc(-c4ccc5oc6ccccc6c5c4)cc(-c4ccc(-c5ccccc5)c5oc6c(-c7ccccc7)cccc6c45)c3)cc2)cc1 ; c1ccc(-c2cccc3c2oc2c(-c4ccccc4)ccc(-c4cc(-c5ccc6c(c5)oc5ccccc56)cc(N(c5ccccc5)c5ccccc5)c4)c23)cc1]

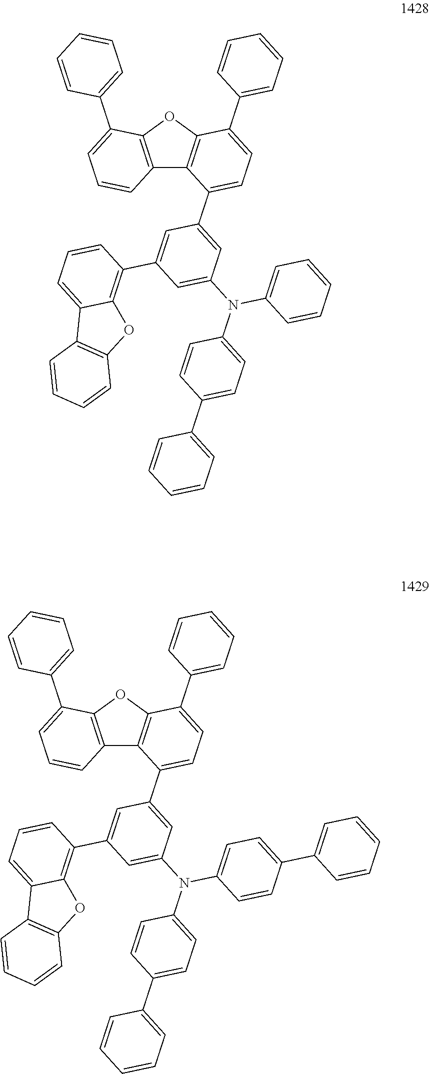 [SMILES: c1ccc(-c2ccc(N(c3ccccc3)c3cc(-c4cccc5c4oc4ccccc45)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4cccc5c4oc4ccccc45)cc(-c4ccc(-c5ccccc5)c5oc6c(-c7ccccc7)cccc6c45)c3)cc2)cc1]

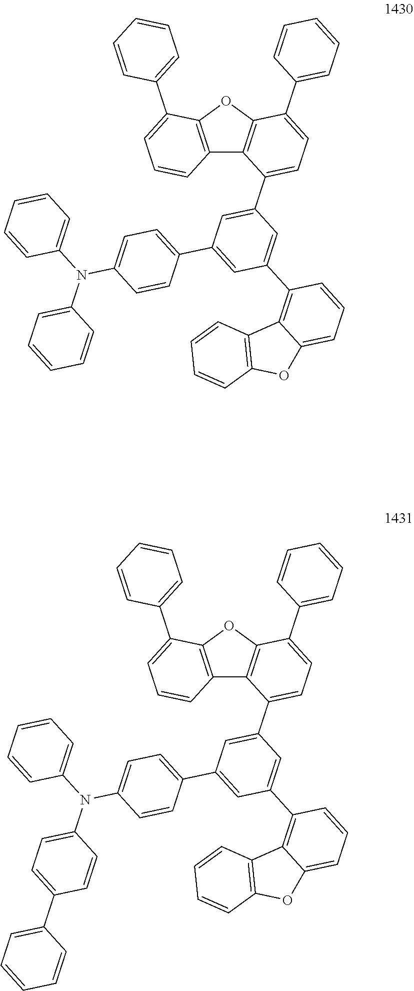 [SMILES: c1ccc(-c2cccc3c2oc2c(-c4ccccc4)ccc(-c4cc(-c5ccc(N(c6ccccc6)c6ccccc6)cc5)cc(-c5cccc6oc7ccccc7c56)c4)c23)cc1 ; c1ccc(-c2ccc(N(c3ccccc3)c3ccc(-c4cc(-c5cccc6oc7ccccc7c56)cc(-c5ccc(-c6ccccc6)c6oc7c(-c8ccccc8)cccc7c56)c4)cc3)cc2)cc1]

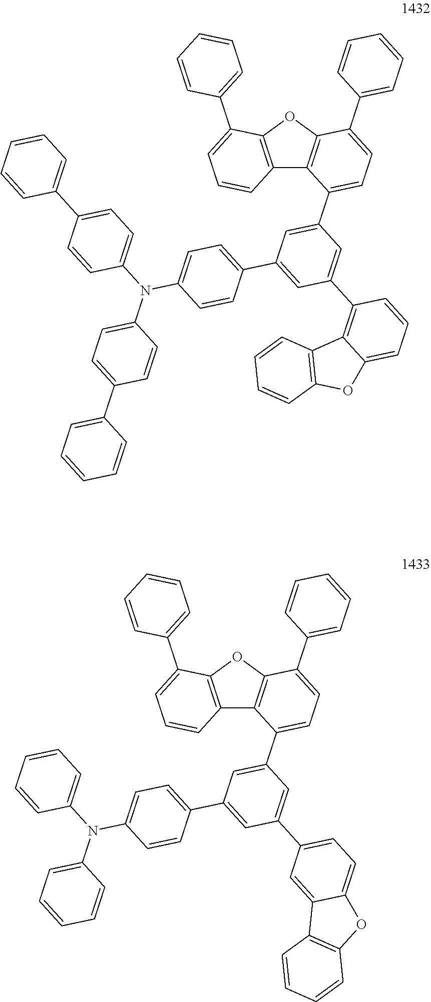 [SMILES: c1ccc(-c2ccc(N(c3ccc(-c4ccccc4)cc3)c3ccc(-c4cc(-c5cccc6oc7ccccc7c56)cc(-c5ccc(-c6ccccc6)c6oc7c(-c8ccccc8)cccc7c56)c4)cc3)cc2)cc1 ; c1ccc(-c2cccc3c2oc2c(-c4ccccc4)ccc(-c4cc(-c5ccc(N(c6ccccc6)c6ccccc6)cc5)cc(-c5ccc6oc7ccccc7c6c5)c4)c23)cc1]

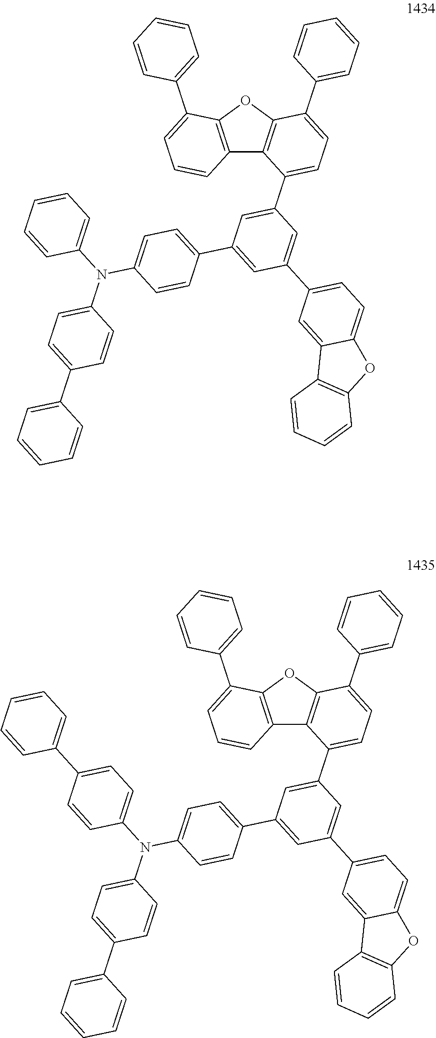 [SMILES: c1ccc(-c2ccc(N(c3ccccc3)c3ccc(-c4cc(-c5ccc6oc7ccccc7c6c5)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5ccc6oc7ccccc7c6c5)cc(-c5ccc(-c6ccccc6)c6oc7c(-c8ccccc8)cccc7c56)c4)cc3)cc2)cc1]

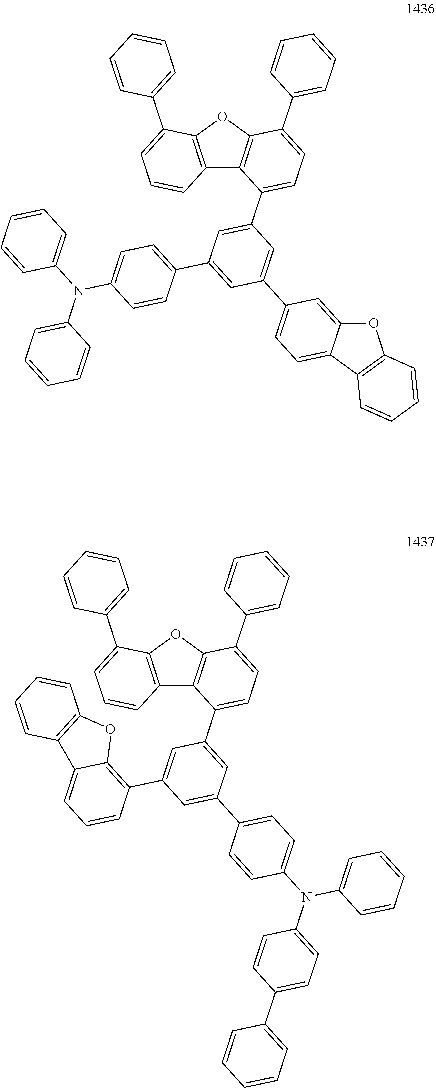 [SMILES: c1ccc(-c2cccc3c2oc2c(-c4ccccc4)ccc(-c4cc(-c5ccc(N(c6ccccc6)c6ccccc6)cc5)cc(-c5ccc6c(c5)oc5ccccc56)c4)c23)cc1 ; c1ccc(-c2ccc(N(c3ccccc3)c3ccc(-c4cc(-c5cccc6c5oc5ccccc56)cc(-c5ccc(-c6ccccc6)c6oc7c(-c8ccccc8)cccc7c56)c4)cc3)cc2)cc1]

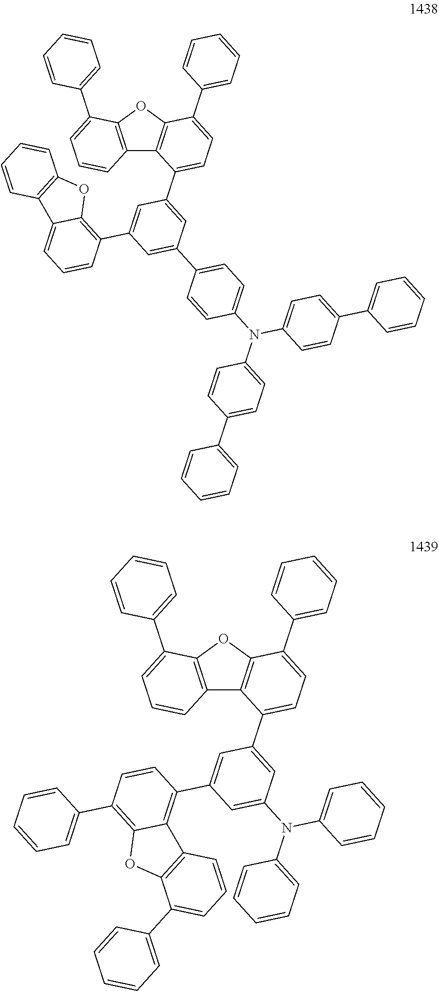 [SMILES: c1ccc(-c2ccc(N(c3ccc(-c4ccccc4)cc3)c3ccc(-c4cc(-c5cccc6c5oc5ccccc56)cc(-c5ccc(-c6ccccc6)c6oc7c(-c8ccccc8)cccc7c56)c4)cc3)cc2)cc1 ; c1ccc(-c2cccc3c2oc2c(-c4ccccc4)ccc(-c4cc(-c5ccc(-c6ccccc6)c6oc7c(-c8ccccc8)cccc7c56)cc(N(c5ccccc5)c5ccccc5)c4)c23)cc1]

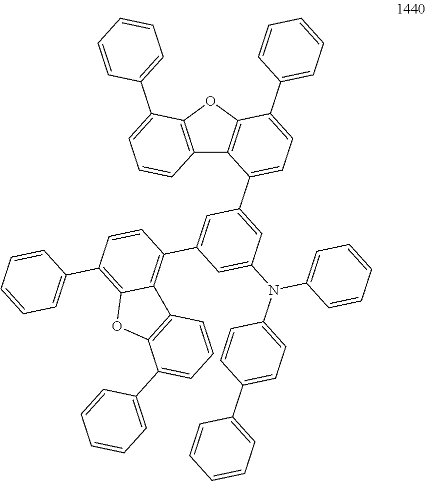 [SMILES: c1ccc(-c2ccc(N(c3ccccc3)c3cc(-c4ccc(-c5ccccc5)c5oc6c(-c7ccccc7)cccc6c45)cc(-c4ccc(-c5ccccc5)c5oc6c(-c7ccccc7)cccc6c45)c3)cc2)cc1]

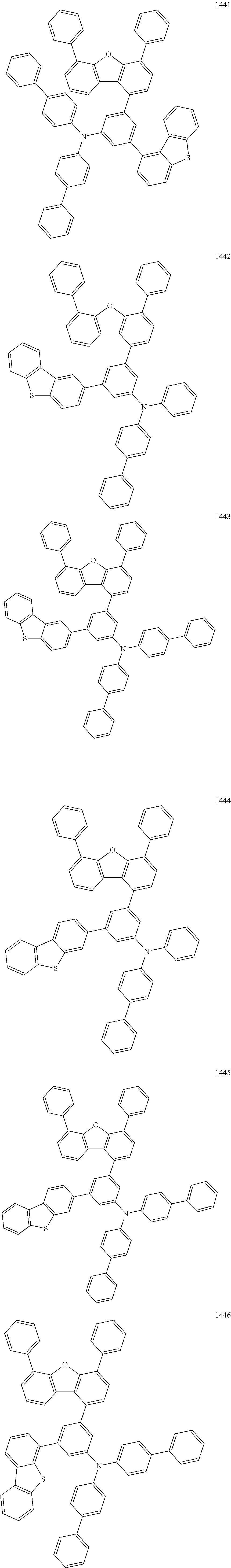

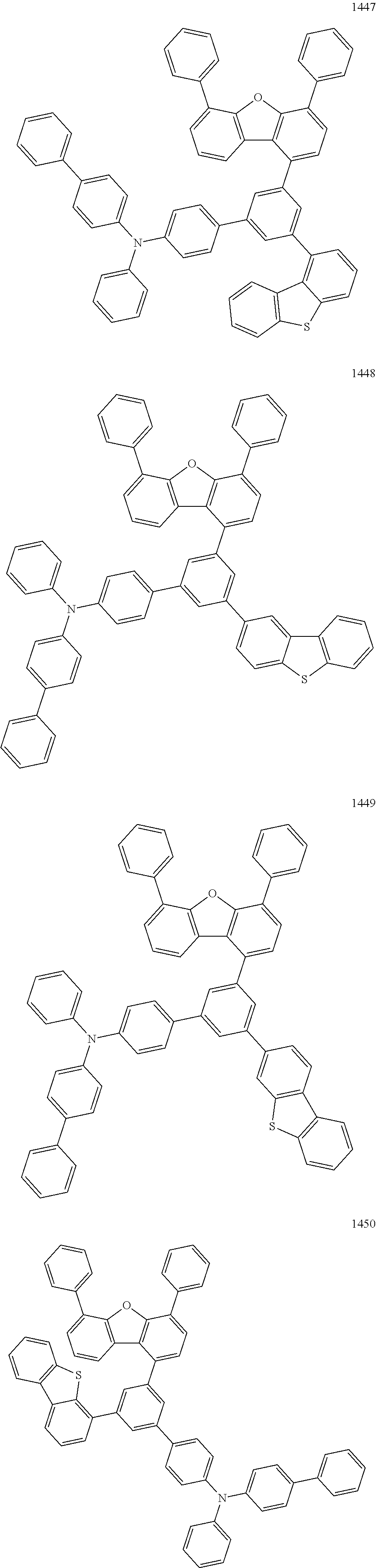 [SMILES: c1ccc(-c2ccc(N(c3ccccc3)c3ccc(-c4cc(-c5cccc6sc7ccccc7c56)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5ccc6sc7ccccc7c6c5)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5ccc6c(c5)sc5ccccc56)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5cccc6c5sc5ccccc56)cc(-c5ccc(-c6ccccc6)c6oc7c(-c8ccccc8)cccc7c56)c4)cc3)cc2)cc1]

Through the characteristic backbone structure and substituents as described above, an organic compound having the inherent characteristics of the backbone structure and the substituents may be synthesized, and for example, when an organic light-emitting device is manufactured, it is possible to manufacture an organic light-emitting compound material that satisfies the conditions required by each organic layer such as a hole transport layer and an electron blocking layer, and in particular, when the compound of [Formula I] according to the present invention is used in an electron blocking layer, and the like, the device characteristics such as the luminous efficiency of the device may be further improved.

The organic light-emitting compound according to the present invention may be used and applied to an organic light-emitting device by a typical manufacturing method. The organic light emitting device according to an exemplary embodiment of the present invention may be composed of a structure including a first electrode, a second electrode and an organic layer disposed therebetween, and may be manufactured using typical device manufacturing methods and materials, except that the organic compound according to the present invention is used in an organic layer of the device.

The organic layer of the organic light-emitting device according to the present invention may be composed of a single-layered structure, but may also be composed of a multi-layered structure in which two or more organic layers are stacked. For example, the organic layer may have a structure including a hole injecting layer, a hole transport layer, a light emitting layer, an electron transport layer, an electron injecting layer, an electron blocking layer, and the like. However, the structure of the organic layer is not limited thereto, and may include a fewer or greater number of the organic layers.

Therefore, in the organic light emitting device according to the present invention, the organic layer may include an electron blocking layer, and at least one layer of the layers may include the organic compound represented by [Formula I].

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, an electron transport layer, an electron injecting layer, and an electron blocking layer hereon, and depositing a cathode material thereon.

In addition to the above methods, the organic light emitting device may be fabricated by depositing a cathode material, organic layer materials, and an anode material in this order on a substrate. The organic layers may have a multilayer structure including a hole injecting layer, a hole transport layer, a light emitting layer, an electron transport layer, an electron injecting layer, and an electron blocking layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO: Al and SnO₂:Sb; and conductive polymers such as poly(3-methylthiophene), poly[3,4-(ethylene-1,2-dioxy)thiophene] (PEDT), polypyrrole, and polyaniline.

As the cathode, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting layer is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport layer is a material that may receive holes transported from the anode or the hole injecting layer and may transfer the holes to the light emitting layer. A material with high hole mobility is suitable. Specific examples thereof include, but are not limited to, arylamine-based organic materials, conductive polymers, and block copolymers consisting of conjugated and non-conjugated segments.

The electron blocking layer is a layer that blocks the movement of electrons and may be formed on the hole transport layer, and may be used to block the movement of electrons without affecting the transport of holes. In addition, on the electron blocking layer, the light-emitting layer may be formed, and the hole blocking layer, the electron transport layer, and the electron injecting layer may be formed.

The hole blocking layer may be used to block the movement of holes without affecting the transport of electrons, and examples of the hole blocking layer are 1,3,5-tri(1-phenyl-1H-benzo[d]imidazol-2-yl)phenyl (TPBi), 2,9-dimethyl4,7-diphenyl-1,10-phenanthroline (BCP), 4,4-bis(N-carbazolyl)-1,1′-biphenyl (CBP), 2-(4-biphenyl)-5-(4-t-butylphenyl)-1,3,4-oxadiazole (PBD), bisbenzimidazo[2,1-a: 1′,2-b′]anthra[2,1,9-def:6,5,10-d′e′f′]diisoguinoline-10,21-dione (PTCBI), 4,7-diphenyl-1,10-phenanthroline (BPhen), or the like, but are not limited thereto.

The light emitting layer is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron injecting layer may be used to have high injection efficiency of electrons transferred from the cathode. Examples of such an electron injecting layer include lithium quinolate (Liq), etc., but are not limited thereto.

The electron transport layer is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light emitting device.

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 5

### (1) Preparation Example 1: Synthesis of Intermediate 5-1

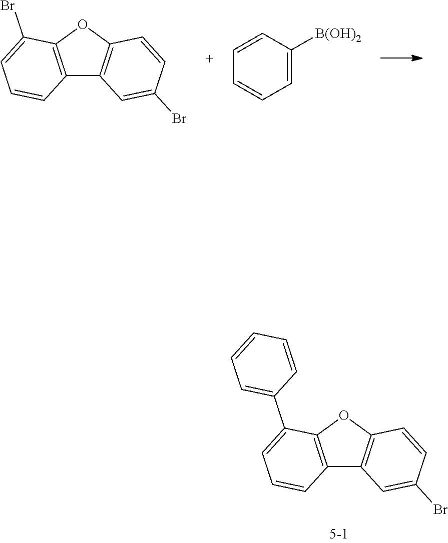 [SMILES: Brc1ccc2oc3c(Br)cccc3c2c1 ; OB(O)c1ccccc1 ; Brc1ccc2oc3c(-c4ccccc4)cccc3c2c1]

200 mL of toluene, 50 mL of ethanol, and 50 mL of H₂O were added to 2,6-dibromodibenzofuran (10.0 g, 0.031 mol), phenylboronic acid (4.5 g, 0.037 mol), K₂CO₃ (12.7 g, 0.092 mol), and Pd(PPh₃)₄ (0.7 g, 0.0006 mol), and the resulting mixture was reacted under stirring at 80° C. for 6 hours. After completion of the reaction, the resulting product was extracted and concentrated, and then subjected to column chromatography to obtain 4.8 g (yield 48.4%) of <Intermediate 5-1>.

### (2) Preparation Example 2: Synthesis of Intermediate 5-2

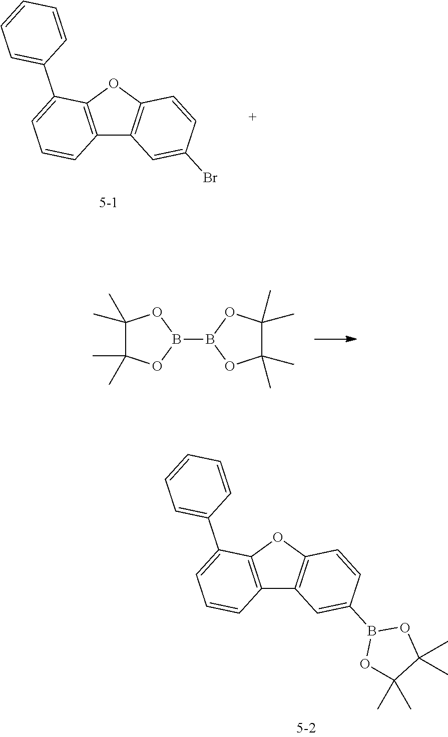

200 mL of dioxane was added to Intermediate 5-1 (10.0 g, 0.031 mol), bis(pinacolato)diboron (9.4 g, 0.037 mol), KOAc (9.1 g, 0.093 mol), and Pd(dppf)Cl₂ (1.1 g, 1.5 mmol), and the resulting mixture was reacted under stirring at 100° C. for 12 hours. After completion of the reaction, the resulting product was extracted and concentrated, and then subjected to column chromatography to obtain 8.4 g (yield 73.3%) of <Intermediate 5-2>.

### (3) Preparation Example 3: Synthesis of Intermediate 5-3

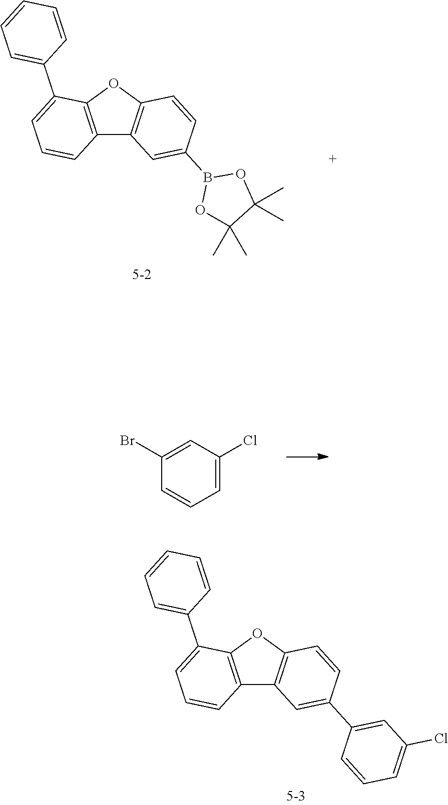 [SMILES: CC1(C)OB(c2ccc3oc4c(-c5ccccc5)cccc4c3c2)OC1(C)C ; Clc1cccc(Br)c1 ; Clc1cccc(-c2ccc3oc4c(-c5ccccc5)cccc4c3c2)c1]

200 mL of toluene, 50 mL of ethanol, and 50 mL of H₂O were added to Intermediate 5-2 (10.0 g, 0.027 mol), 1-bromo-3-chlorobenzene (6.2 g, 0.032 mol), K₂CO₃ (11.2 g, 0.081 mol), and Pd(PPh₃)₄ (0.6 g, 0.0005 mol), and the resulting mixture was reacted under stirring at 80° C. for 6 hours. After completion of the reaction, the resulting product was extracted and concentrated, and then subjected to column chromatography and recrystallization to obtain 7.1 g (yield 74.1%) of <Intermediate 5-3>.

### (4) Preparation Example 4: Synthesis of Compound 5

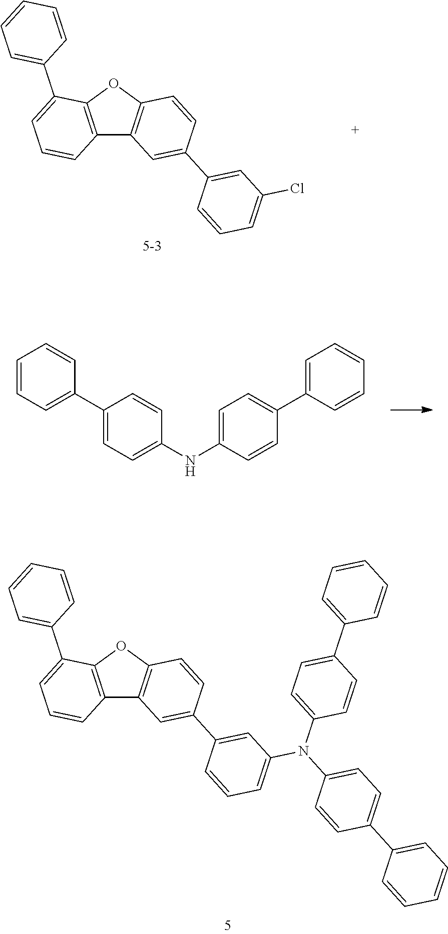 [SMILES: Clc1cccc(-c2ccc3oc4c(-c5ccccc5)cccc4c3c2)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5oc6c(-c7ccccc7)cccc6c5c4)c3)cc2)cc1]

150 mL of xylene was added to Intermediate 5-3 (10.0 g, 0.028 mol), N-[1,1′-biphenyl]-4-yl[1,1′-biphenyl]-4-amine (13.6 g, 0.042 mol), NaOtBu (8.1 g, 0.085 mol), Pd(dba)₂ (0.7 g, 1.1 mmol), and t-Bu₃P (0.5 g, 2.3 mmol), and the resulting mixture was reacted under stirring at 120° C. for 4 hours. After completion of the reaction, the resulting product was extracted and concentrated, and then subjected to column chromatography and recrystallization to obtain 13.3 g (yield 73.8%) of <Compound 5>.

LC/MS: m/z=639 [(M)⁺]

### Synthesis Example 2: Synthesis of Compound 11

### (1) Preparation Example 1: Synthesis of Compound 11

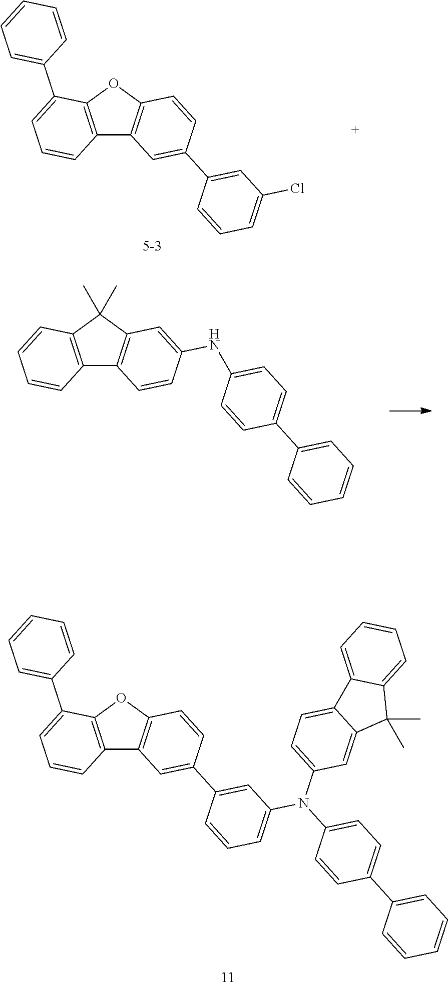 [SMILES: Clc1cccc(-c2ccc3oc4c(-c5ccccc5)cccc4c3c2)c1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3cccc(-c4ccc5oc6c(-c7ccccc7)cccc6c5c4)c3)cc21]

150 mL of xylene was added to Intermediate 5-3 (10.0 g, 0.028 mol), N-[1,1′-biphenyl]-4-yl-9,9-dimethyl-9H-fluoren-2-amine (15.3 g, 0.042 mol), NaOtBu (8.1 g, 0.085 mol), Pd(dba)₂ (0.7 g, 1.1 mmol), and t-Bu₃P (0.5 g, 2.3 mmol), and the resulting mixture was reacted under stirring at 120° C. for 4 hours. After completion of the reaction, the resulting product was extracted and concentrated, and then subjected to column chromatography and recrystallization to obtain 13.8 g (yield 72.0%) of <Compound 11>.

LC/MS: m/z=679 [(M)⁺]

### Synthesis Example 3: Synthesis of Compound 26

### (1) Preparation Example 1: Synthesis of Compound 26

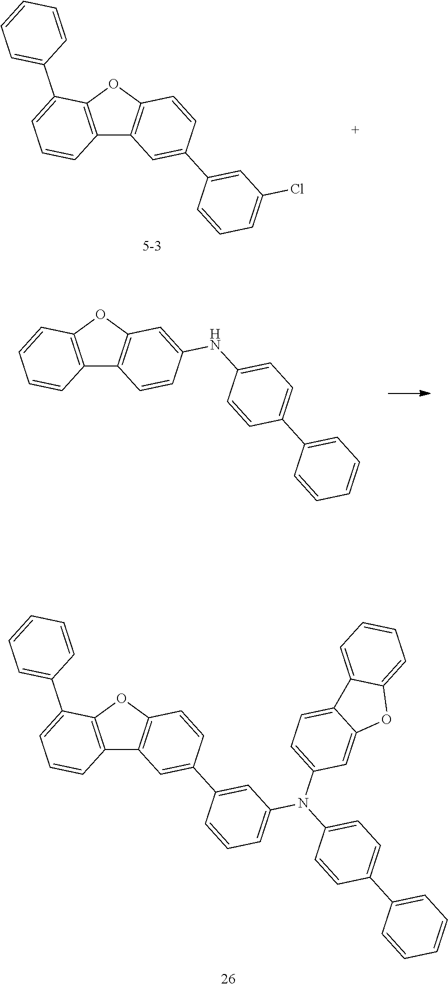 [SMILES: Clc1cccc(-c2ccc3oc4c(-c5ccccc5)cccc4c3c2)c1 ; c1ccc(-c2ccc(Nc3ccc4c(c3)oc3ccccc34)cc2)cc1 ; c1ccc(-c2ccc(N(c3cccc(-c4ccc5oc6c(-c7ccccc7)cccc6c5c4)c3)c3ccc4c(c3)oc3ccccc34)cc2)cc1]

150 mL of xylene was added to Intermediate 5-3 (10.0 g, 0.028 mol), N-[1,1′-biphenyl]-4-yl-3-dibenzofuranamine (14.2 g, 0.042 mol), NaOtBu (8.1 g, 0.085 mol), Pd(dba)₂ (0.7 g, 1.1 mmol), and t-Bu₃P (0.5 g, 2.3 mmol), and the resulting mixture was reacted under stirring at 120° C. for 4 hours. After completion of the reaction, the resulting product was extracted and concentrated, and then subjected to column chromatography and recrystallization to obtain 12.9 g (yield 70.0%) of <Compound 26>.

LC/MS: m/z=653 [(M)⁺]

### Synthesis Example 4: Synthesis of Compound 41

### (1) Preparation Example 1: Synthesis of Compound 41

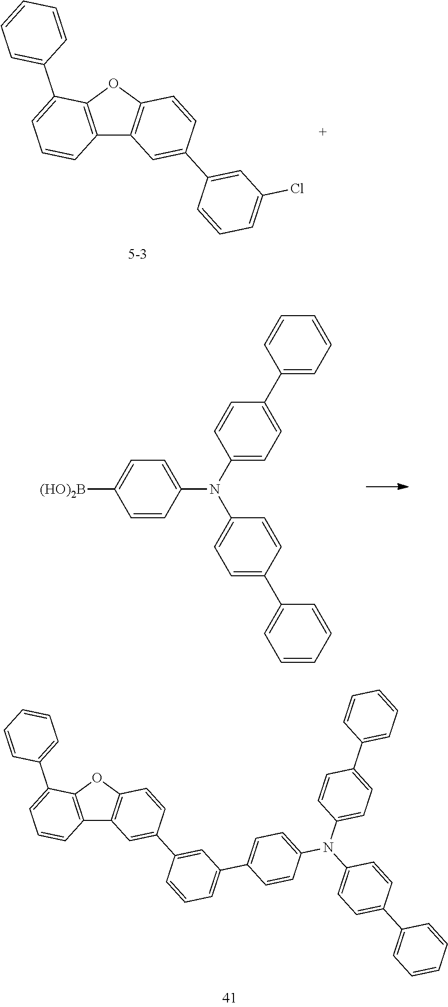 [SMILES: Clc1cccc(-c2ccc3oc4c(-c5ccccc5)cccc4c3c2)c1 ; OB(O)c1ccc(N(c2ccc(-c3ccccc3)cc2)c2ccc(-c3ccccc3)cc2)cc1 ; c1ccc(-c2ccc(N(c3ccc(-c4ccccc4)cc3)c3ccc(-c4cccc(-c5ccc6oc7c(-c8ccccc8)cccc7c6c5)c4)cc3)cc2)cc1]

Intermediate 5-3 (10.0 g, 0.028 mol), B-[4-[bis([1,1′-biphenyl]-4-yl)amino]phenyl]boronic acid (14.9 g, 0.034 mol), K₂CO₃ (11.7 g, 0.085 mol), Pd(OAc)₂ (1.6 g, 1.4 mmol), X-Phos (1.3 g, 2.8 mmol), 200 mL of THF, and 50 mL of H₂O were put into a container, and the resulting mixture was reacted under stirring at 120° C. for 6 hours. After completion of the reaction, the resulting product was extracted and concentrated, and then subjected to column chromatography and recrystallization to obtain 12.1 g (yield 60.0%) of <Compound 41>.

LC/MS: m/z=715 [(M)⁺]

### Synthesis Example 5: Synthesis of Compound 54

### (1) Preparation Example 1: Synthesis of Intermediate 54-1

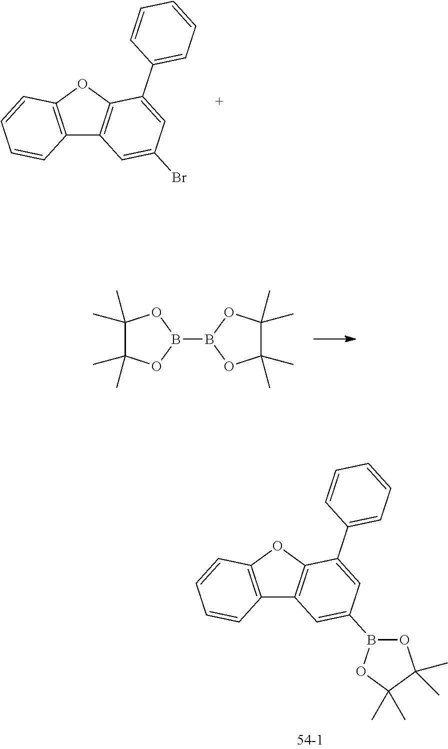

200 mL of dioxane was added to 2-bromo-4-phenyldibenzofuran (10.0 g, 0.031 mol), bis(pinacolato)diboron (9.4 g, 0.037 mol), KOAc (9.1 g, 0.093 mol), and Pd(dppf)Cl₂ (1.1 g, 1.5 mmol), and the resulting mixture was reacted under stirring at 100° C. for 12 hours. After completion of the reaction, the resulting product was extracted and concentrated, and then subjected to column chromatography to obtain 8.1 g (yield 70.7%) of <Intermediate 54-1>.

### (2) Preparation Example 2: Synthesis of Intermediate 54-2

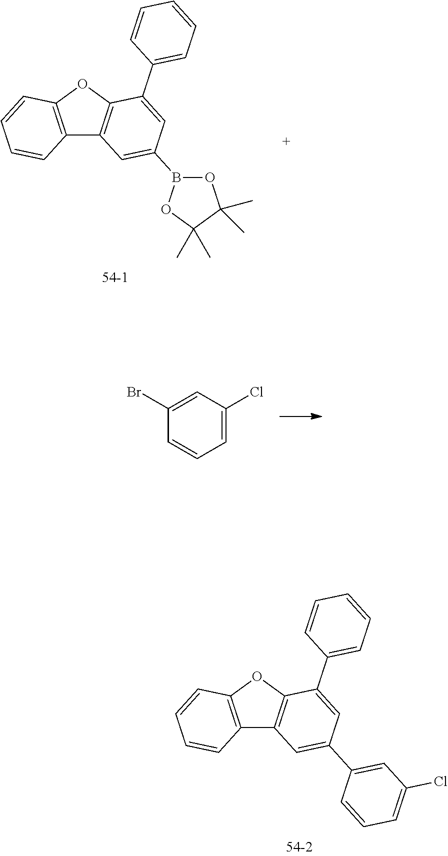 [SMILES: CC1(C)OB(c2cc(-c3ccccc3)c3oc4ccccc4c3c2)OC1(C)C ; Clc1cccc(Br)c1 ; Clc1cccc(-c2cc(-c3ccccc3)c3oc4ccccc4c3c2)c1]

200 mL of toluene, 50 mL of ethanol, and 50 mL of H₂O were added to Intermediate 54-1 (10.0 g, 0.027 mol), 1-bromo-3-chlorobenzene (6.2 g, 0.032 mol), K₂CO₃ (11.2 g, 0.081 mol), and Pd(PPh₃)₄ (0.6 g, 0.0005 mol), and the resulting mixture was reacted under stirring at 80° C. for 6 hours. After completion of the reaction, the resulting product was extracted and concentrated, and then subjected to column chromatography and recrystallization to obtain 6.2 g (yield 64.7%) of <Intermediate 54-2>.

### (3) Preparation Example 3: Synthesis of Compound 54

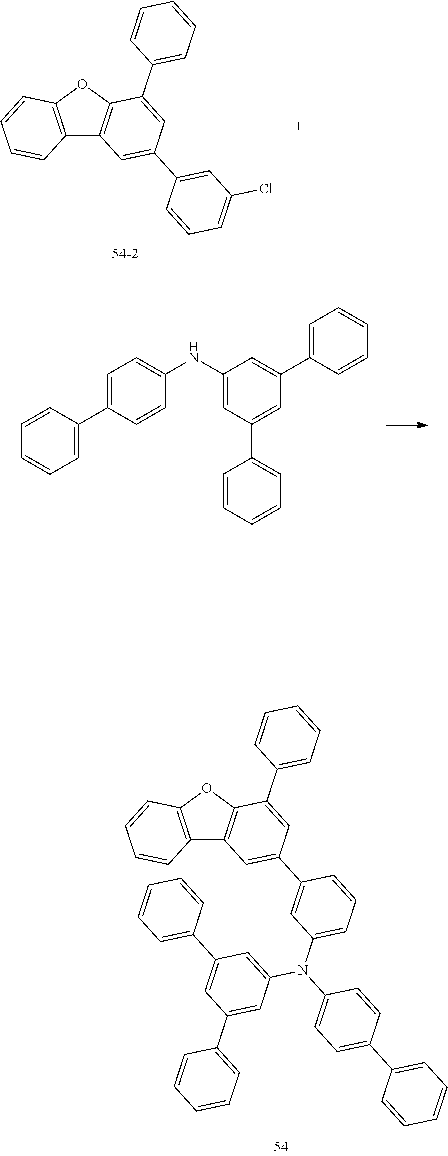 [SMILES: Clc1cccc(-c2cc(-c3ccccc3)c3oc4ccccc4c3c2)c1 ; c1ccc(-c2ccc(Nc3cc(-c4ccccc4)cc(-c4ccccc4)c3)cc2)cc1 ; c1ccc(-c2ccc(N(c3cccc(-c4cc(-c5ccccc5)c5oc6ccccc6c5c4)c3)c3cc(-c4ccccc4)cc(-c4ccccc4)c3)cc2)cc1]

150 mL of xylene was added to Intermediate 54-2 (10.0 g, 0.028 mol), N-[1,1′-biphenyl]-4-yl[1,1′: 3′,1″-terphenyl]-5′-amine (16.8 g, 0.042 mol), NaOtBu (8.1 g, 0.085 mol), Pd(dba)₂ (0.7 g, 1.1 mmol), and t-Bu₃P (0.5 g, 2.3 mmol), and the resulting mixture was reacted under stirring at 120° C. for 4 hours. After completion of the reaction, the resulting product was extracted and concentrated, and then subjected to column chromatography and recrystallization to obtain 12.4 g (yield 61.5%) of <Compound 54>. LC/MS: m/z=715 [(M)⁺]

### Synthesis Example 6: Synthesis of Compound 68

### (1) Preparation Example 1: Synthesis of Compound 68

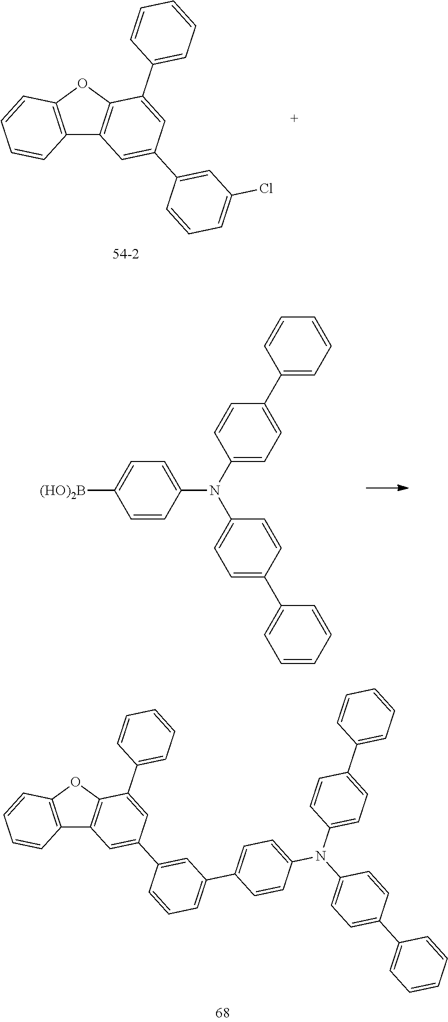

Intermediate 54-2 (10.0 g, 0.028 mol), B-[4-[bis([1,1′-biphenyl]-4-yl)amino]phenyl]boronic acid (14.9 g, 0.034 mol), K₂CO₃ (11.7 g, 0.085 mol), Pd(OAc)₂ (1.6 g, 1.4 mmol), X-Phos (1.3 g, 2.8 mmol), 200 mL of THF, and 50 mL of H₂O were put into a container, and the resulting mixture was reacted under stirring at 120° C. for 6 hours. After completion of the reaction, the resulting product was extracted and concentrated, and then subjected to column chromatography and recrystallization to obtain 11.3 g (yield 56.0%) of <Compound 68>.

LC/MS: m/z=715 [(M)⁺]

### Synthesis Example 7: Synthesis of Compound 76

### (1) Preparation Example 1: Synthesis of Intermediate 76-1

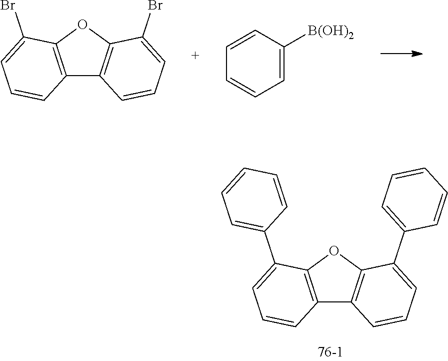 [SMILES: Brc1cccc2c1oc1c(Br)cccc12 ; OB(O)c1ccccc1 ; c1ccc(-c2cccc3c2oc2c(-c4ccccc4)cccc23)cc1]

200 mL of toluene, 50 mL of ethanol, and 50 mL of H₂O were added to 4,6-dibromodibenzofuran (10.0 g, 0.031 mol), phenylboronic acid (9.0 g, 0.074 mol), K₂CO₃ (25.4 g, 0.184 mol), and Pd(PPh₃)₄ (0.7 g, 0.0006 mol), and the resulting mixture was reacted under stirring at 80° C. for 6 hours. After completion of the reaction, the resulting product was extracted and concentrated, and then subjected to column chromatography to obtain 7.6 g (yield 77.3%) of <Intermediate 76-1>.

### (2) Preparation Example 2: Synthesis of Intermediate 76-2

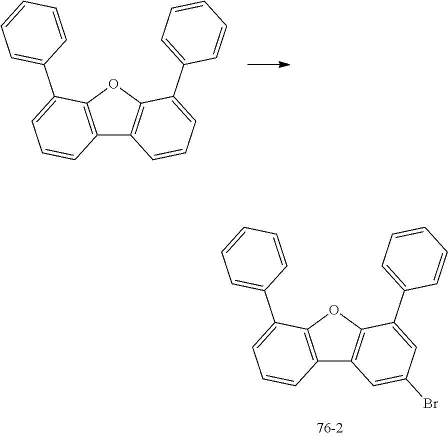 [SMILES: c1ccc(-c2cccc3c2oc2c(-c4ccccc4)cccc23)cc1 ; Brc1cc(-c2ccccc2)c2oc3c(-c4ccccc4)cccc3c2c1]

After Intermediate 76-1 (10.0 g, 0.031 mol) was dissolved in 100 mL of CHCl₃, bromine (in CHCl₃, 12.5 g, 0.078 mol) was slowly added dropwise thereto, and the resulting mixture was reacted under stirring at room temperature for 12 hours. After completion of the reaction, the resulting product was extracted and concentrated, and then subjected to column chromatography to obtain 7.5 g (yield 60.2%) of <Intermediate 76-2>.

### (3) Preparation Example 3: Synthesis of Intermediate 76-3

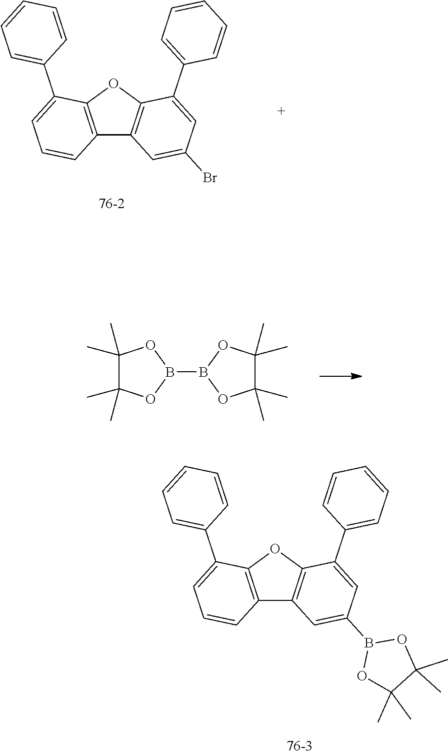

200 mL of dioxane was added to Intermediate 76-2 (10.0 g, 0.025 mol), bis(pinacolato)diboron (7.6 g, 0.030 mol), KOAc (7.4 g, 0.075 mol), and Pd(dppf)Cl₂ (0.9 g, 1.3 mmol), and the resulting mixture was reacted under stirring at 100° C. for 12 hours. After completion of the reaction, the resulting product was extracted and concentrated, and then subjected to column chromatography to obtain 8.2 g (yield 73.4%) of <Intermediate 76-3>.

### (4) Preparation Example 4: Synthesis of Intermediate 76-4

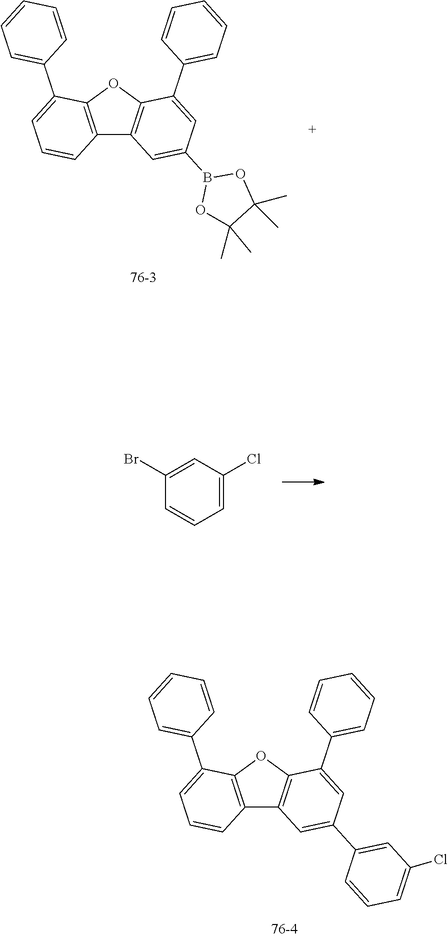 [SMILES: CC1(C)OB(c2cc(-c3ccccc3)c3oc4c(-c5ccccc5)cccc4c3c2)OC1(C)C ; Clc1cccc(Br)c1 ; Clc1cccc(-c2cc(-c3ccccc3)c3oc4c(-c5ccccc5)cccc4c3c2)c1]

200 mL of toluene, 50 mL of ethanol, and 50 mL of H₂O were added to Intermediate 76-3 (10.0 g, 0.022 mol), 1-bromo-3-chlorobenzene (5.2 g, 0.027 mol), K₂CO₃ (9.3 g, 0.067 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 6.5 g (yield 67.3%) of <Intermediate 76-4>.

### (5) Preparation Example 5: Synthesis of Compound 76

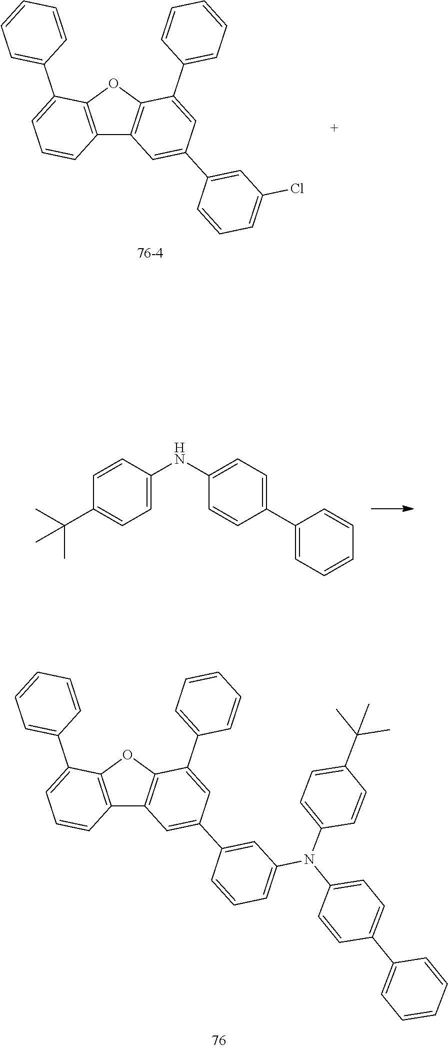 [SMILES: Clc1cccc(-c2cc(-c3ccccc3)c3oc4c(-c5ccccc5)cccc4c3c2)c1 ; CC(C)(C)c1ccc(Nc2ccc(-c3ccccc3)cc2)cc1 ; CC(C)(C)c1ccc(N(c2ccc(-c3ccccc3)cc2)c2cccc(-c3cc(-c4ccccc4)c4oc5c(-c6ccccc6)cccc5c4c3)c2)cc1]

150 mL of xylene was added to Intermediate 76-4 (10.0 g, 0.023 mol), N-[4-(1,1-dimethylethyl)phenyl][1,1′-biphenyl]-4-amine (10.5 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 10.4 g (yield 64.4%) of <Compound 76>.

LC/MS: m/z=695 [(M)⁺]

### Synthesis Example 8: Synthesis of Compound 81

### (1) Preparation Example 1: Synthesis of Compound 81

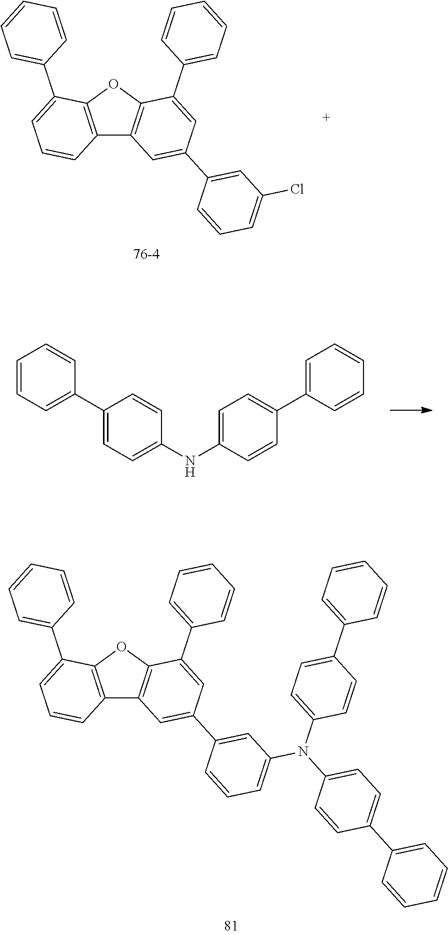 [SMILES: Clc1cccc(-c2cc(-c3ccccc3)c3oc4c(-c5ccccc5)cccc4c3c2)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4cc(-c5ccccc5)c5oc6c(-c7ccccc7)cccc6c5c4)c3)cc2)cc1]

150 mL of xylene was added to Intermediate 76-4 (10.0 g, 0.023 mol), bis(4-biphenylyl)amine (11.2 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 12.9 g (yield 77.7%) of <Compound 81>.

LC/MS: m/z=715 [(M)⁺]

### Synthesis Example 9: Synthesis of Compound 89

### (1) Preparation Example 1: Synthesis of Compound 89

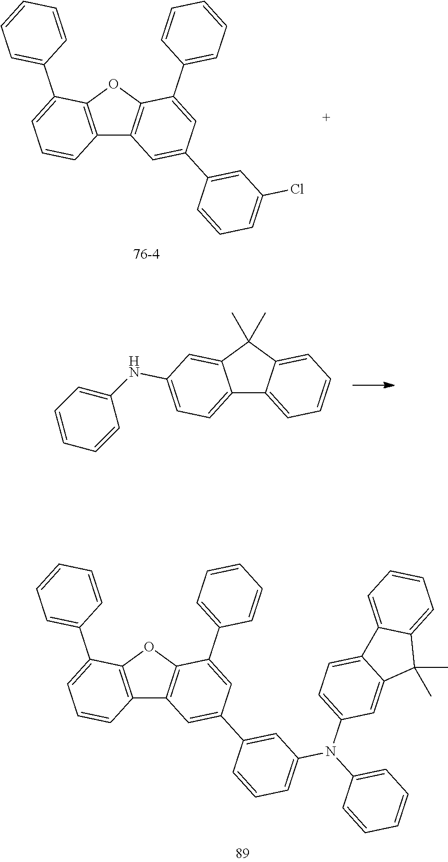 [SMILES: Clc1cccc(-c2cc(-c3ccccc3)c3oc4c(-c5ccccc5)cccc4c3c2)c1 ; CC1(C)c2ccccc2-c2ccc(Nc3ccccc3)cc21 ; CC1(C)c2ccccc2-c2ccc(N(c3ccccc3)c3cccc(-c4cc(-c5ccccc5)c5oc6c(-c7ccccc7)cccc6c5c4)c3)cc21]

150 mL of xylene was added to Intermediate 76-4 (10.0 g, 0.023 mol), 2-(phenylamino)-9,9-dimethylfluorene (9.9 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 10.8 g (yield 68.5%) of <Compound 89>. LC/MS: m/z=679 [(M)⁺]

### Synthesis Example 10: Synthesis of Compound 91

### (1) Preparation Example 1: Synthesis of Compound 91

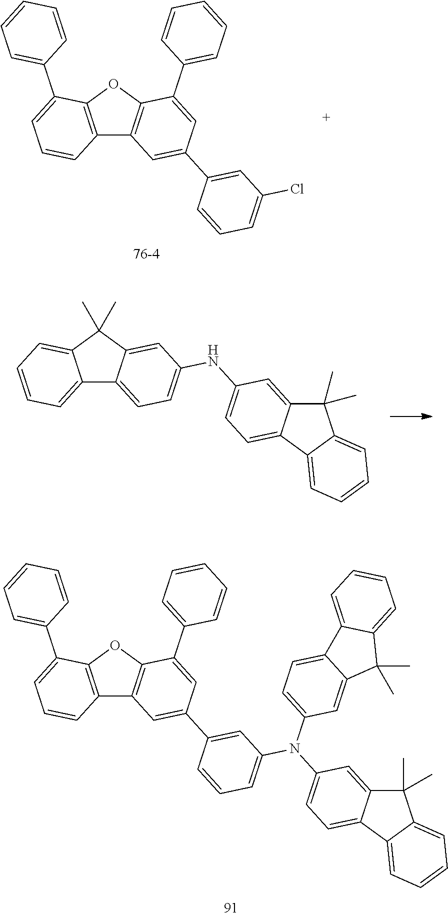 [SMILES: Clc1cccc(-c2cc(-c3ccccc3)c3oc4c(-c5ccccc5)cccc4c3c2)c1 ; CC1(C)c2ccccc2-c2ccc(Nc3ccc4c(c3)C(C)(C)c3ccccc3-4)cc21 ; CC1(C)c2ccccc2-c2ccc(N(c3cccc(-c4cc(-c5ccccc5)c5oc6c(-c7ccccc7)cccc6c5c4)c3)c3ccc4c(c3)C(C)(C)c3ccccc3-4)cc21]

150 mL of xylene was added to Intermediate 76-4 (10.0 g, 0.023 mol), bis(9,9-dimethyl-9H-fluoren-2-yl)amine (14.0 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.5 g (yield 62.3%) of <Compound 91>.

LC/MS: m/z=795 [(M)⁺]

### Synthesis Example 11: Synthesis of Compound 105

### (1) Preparation Example 1: Synthesis of Compound 105

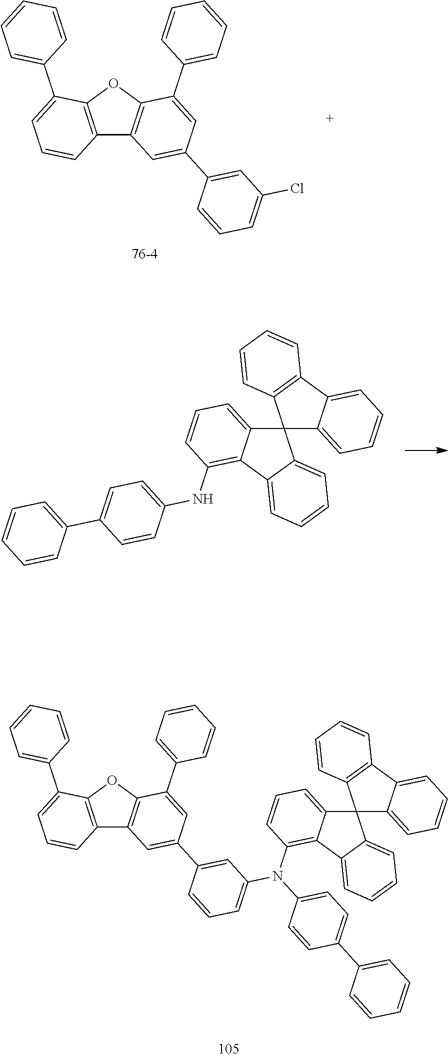 [SMILES: Clc1cccc(-c2cc(-c3ccccc3)c3oc4c(-c5ccccc5)cccc4c3c2)c1 ; c1ccc(-c2ccc(Nc3cccc4c3-c3ccccc3C43c4ccccc4-c4ccccc43)cc2)cc1 ; c1ccc(-c2ccc(N(c3cccc(-c4cc(-c5ccccc5)c5oc6c(-c7ccccc7)cccc6c5c4)c3)c3cccc4c3-c3ccccc3C43c4ccccc4-c4ccccc43)cc2)cc1]

150 mL of xylene was added to Intermediate 76-4 (10.0 g, 0.023 mol), N-[1,1′-biphenyl]-4-yl-9,9′-spirobi[9H-fluoren]-4-amine (16.8 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 10.9 g (yield 53.5%) of <Compound 105>.

LC/MS: m/z=877 [(M)⁺]

### Synthesis Example 12: Synthesis of Compound 110

### (1) Preparation Example 1: Synthesis of Compound 110

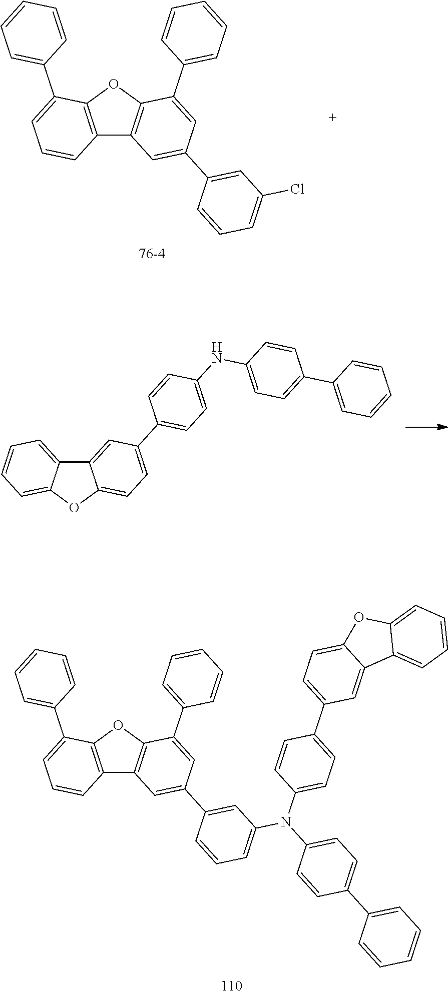 [SMILES: Clc1cccc(-c2cc(-c3ccccc3)c3oc4c(-c5ccccc5)cccc4c3c2)c1 ; c1ccc(-c2ccc(Nc3ccc(-c4ccc5oc6ccccc6c5c4)cc3)cc2)cc1 ; c1ccc(-c2ccc(N(c3ccc(-c4ccc5oc6ccccc6c5c4)cc3)c3cccc(-c4cc(-c5ccccc5)c5oc6c(-c7ccccc7)cccc6c5c4)c3)cc2)cc1]

150 mL of xylene was added to Intermediate 76-4 (10.0 g, 0.023 mol), N-[4-(2-dibenzofuranyl)phenyl][1,1′-biphenyl]-4-amine (14.3 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.0 g (yield 58.8%) of <Compound 110>.

LC/MS: m/z=805 [(M)⁺]

### Synthesis Example 13: Synthesis of Compound 112

### (1) Preparation Example 1: Synthesis of Intermediate 112-1

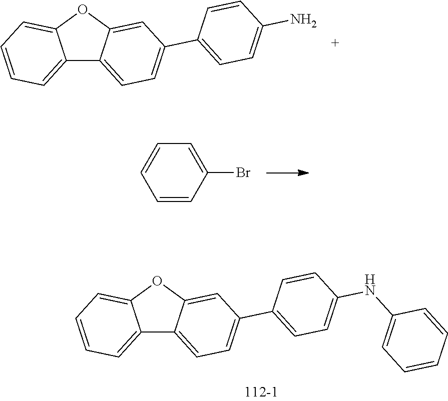 [SMILES: Nc1ccc(-c2ccc3c(c2)oc2ccccc23)cc1 ; Brc1ccccc1 ; c1ccc(Nc2ccc(-c3ccc4c(c3)oc3ccccc34)cc2)cc1]

150 mL of xylene was added to 4-(3-dibenzofuranyl)benzenamine (10.0 g, 0.039 mol), bromobenzene (9.1 g, 0.058 mol), NaOtBu (11.1 g, 0.116 mol), Pd(dba)₂ (0.9 g, 1.5 mmol), and t-Bu₃P (0.6 g, 3.1 mmol), and the resulting mixture was reacted under stirring at 70° C. for 4 hours. After completion of the reaction, the resulting product was extracted and concentrated, and then subjected to column chromatography and recrystallization to obtain 8.8 g (yield 68.0%) of <Intermediate 112-1>.

### (2) Preparation Example 2: Synthesis of Compound 112

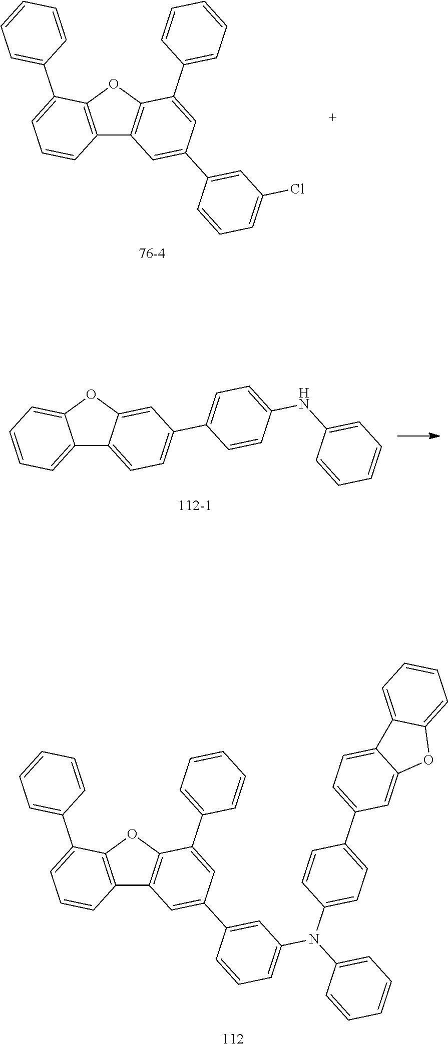 [SMILES: Clc1cccc(-c2cc(-c3ccccc3)c3oc4c(-c5ccccc5)cccc4c3c2)c1 ; c1ccc(Nc2ccc(-c3ccc4c(c3)oc3ccccc34)cc2)cc1 ; c1ccc(-c2cccc3c2oc2c(-c4ccccc4)cc(-c4cccc(N(c5ccccc5)c5ccc(-c6ccc7c(c6)oc6ccccc67)cc5)c4)cc23)cc1]

150 mL of xylene was added to Intermediate 76-4 (10.0 g, 0.023 mol), Intermediate 112-1 (11.7 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.0 g (yield 65.0%) of <Compound 112>.

LC/MS: m/z=729 [(M)⁺]

### Synthesis Example 14: Synthesis of Compound 117

### (1) Preparation Example 1: Synthesis of Intermediate 117-1

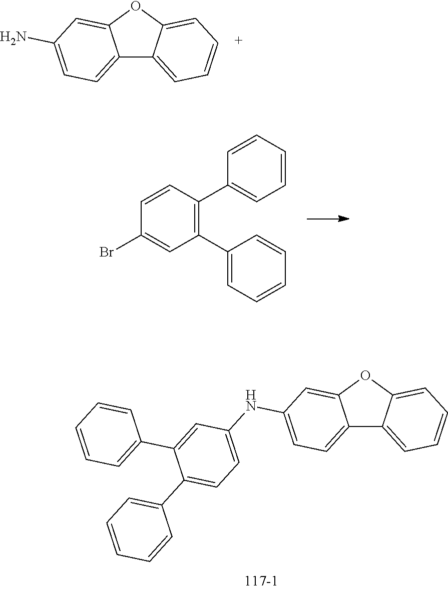 [SMILES: Nc1ccc2c(c1)oc1ccccc12 ; Brc1ccc(-c2ccccc2)c(-c2ccccc2)c1 ; c1ccc(-c2ccc(Nc3ccc4c(c3)oc3ccccc34)cc2-c2ccccc2)cc1]

150 mL of xylene was added to dibenzo[b,d]furan-3-amine (10.0 g, 0.055 mol), 4′-bromo-1,1′: 2′,1″-terphenyl (25.3 g, 0.082 mol), NaOtBu (15.7 g, 0.164 mol), Pd(dba)₂ (1.3 g, 2.2 mmol), and t-Bu₃P (0.9 g, 4.4 mmol), and the resulting mixture was reacted under stirring at 70° C. for 4 hours. After completion of the reaction, the resulting product was extracted and concentrated, and then subjected to column chromatography and recrystallization to obtain 9.5 g (yield 57.4%) of <Intermediate 117-1>.

### (2) Preparation Example 2: Synthesis of Compound 117

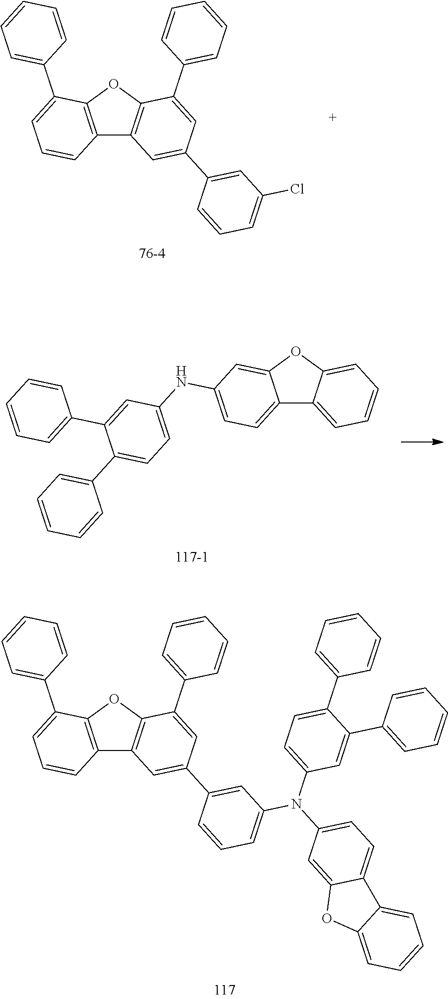 [SMILES: Clc1cccc(-c2cc(-c3ccccc3)c3oc4c(-c5ccccc5)cccc4c3c2)c1 ; c1ccc(-c2ccc(Nc3ccc4c(c3)oc3ccccc34)cc2-c2ccccc2)cc1 ; c1ccc(-c2ccc(N(c3cccc(-c4cc(-c5ccccc5)c5oc6c(-c7ccccc7)cccc6c5c4)c3)c3ccc4c(c3)oc3ccccc34)cc2-c2ccccc2)cc1]

150 mL of xylene was added to Intermediate 76-4 (10.0 g, 0.023 mol), Intermediate 117-1 (14.3 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 12.7 g (yield 67.9%) of <Compound 117>.

LC/MS: m/z=805 [(M)⁺]

### Synthesis Example 15: Synthesis of Compound 119

### (1) Preparation Example 1: Synthesis of Compound 119

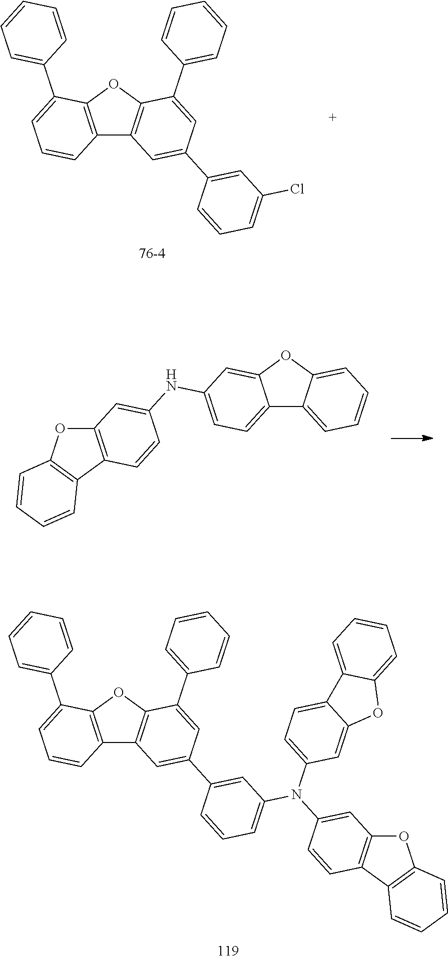 [SMILES: Clc1cccc(-c2cc(-c3ccccc3)c3oc4c(-c5ccccc5)cccc4c3c2)c1 ; c1ccc2c(c1)oc1cc(Nc3ccc4c(c3)oc3ccccc34)ccc12 ; c1ccc(-c2cccc3c2oc2c(-c4ccccc4)cc(-c4cccc(N(c5ccc6c(c5)oc5ccccc56)c5ccc6c(c5)oc5ccccc56)c4)cc23)cc1]

150 mL of xylene was added to Intermediate 76-4 (10.0 g, 0.023 mol), N-3-dibenzofuranyl-3-dibenzofuranamine (12.2 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 10.7 g (yield 62.0%) of <Compound 119>. LC/MS: m/z=743 [(M)⁺]

### Synthesis Example 16: Synthesis of Compound 121

### (1) Preparation Example 1: Synthesis of Compound 121

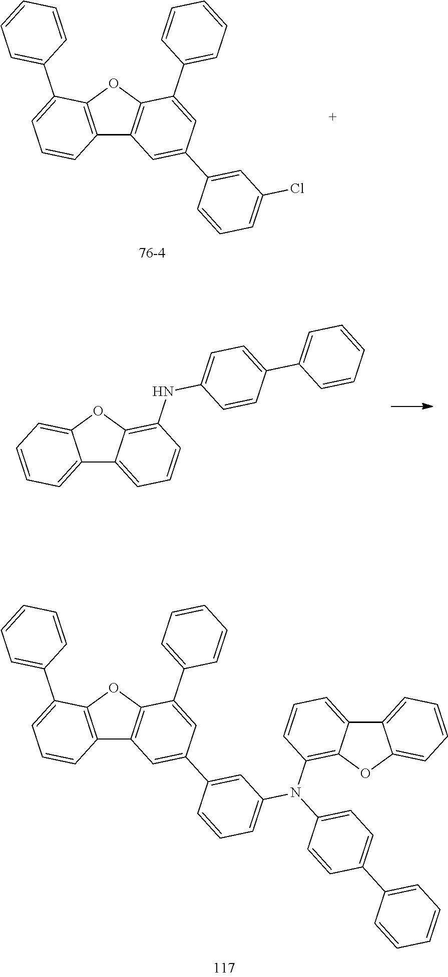 [SMILES: Clc1cccc(-c2cc(-c3ccccc3)c3oc4c(-c5ccccc5)cccc4c3c2)c1 ; c1ccc(-c2ccc(Nc3cccc4c3oc3ccccc34)cc2)cc1 ; c1ccc(-c2ccc(N(c3cccc(-c4cc(-c5ccccc5)c5oc6c(-c7ccccc7)cccc6c5c4)c3)c3cccc4c3oc3ccccc34)cc2)cc1]

150 mL of xylene was added to Intermediate 76-4 (10.0 g, 0.023 mol), N-[1,1′-biphenyl]-4-yl-4-dibenzofuranamine (11.7 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.3 g (yield 66.7%) of <Compound 121>. LC/MS: m/z=729 [(M)⁺]

### Synthesis Example 17: Synthesis of Compound 124

### (1) Preparation Example 1: Synthesis of Intermediate 124-1

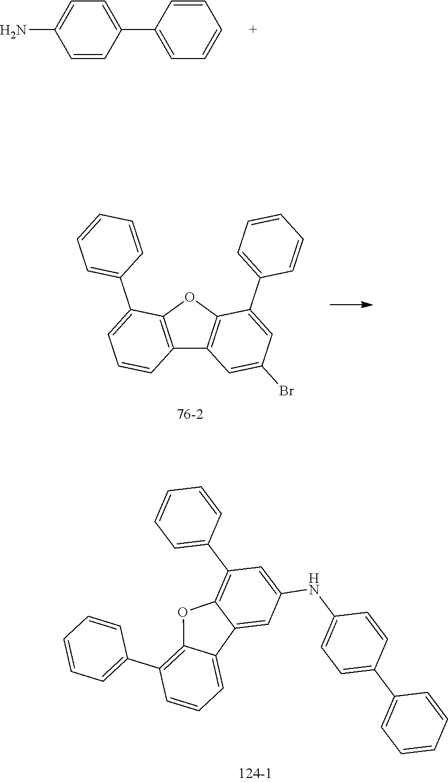 [SMILES: Nc1ccc(-c2ccccc2)cc1 ; Brc1cc(-c2ccccc2)c2oc3c(-c4ccccc4)cccc3c2c1 ; c1ccc(-c2ccc(Nc3cc(-c4ccccc4)c4oc5c(-c6ccccc6)cccc5c4c3)cc2)cc1]

150 mL of xylene was added to 4-aminobiphenyl (10.0 g, 0.059 mol), Intermediate 76-2 (35.4 g, 0.089 mol), NaOtBu (17.0 g, 0.177 mol), Pd(dba)₂ (1.4 g, 2.4 mmol), and t-Bu₃P (1.0 g, 4.7 mmol), and the resulting mixture was reacted under stirring at 120° C. for 4 hours. After completion of the reaction, the resulting product was extracted and concentrated, and then subjected to column chromatography and recrystallization to obtain 15.1 g (yield 52.4%) of <Intermediate 124-1>.

### (2) Preparation Example 2: Synthesis of Compound 124

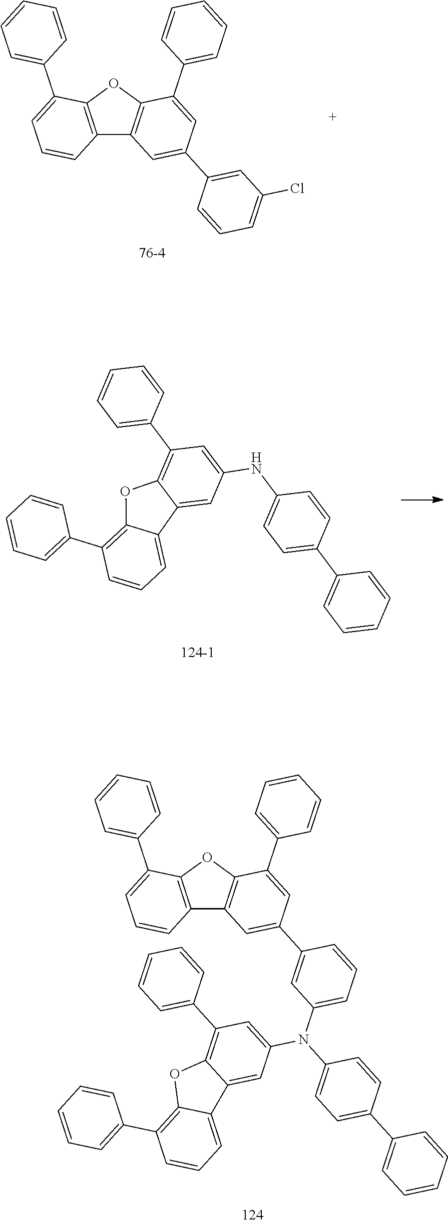 [SMILES: Clc1cccc(-c2cc(-c3ccccc3)c3oc4c(-c5ccccc5)cccc4c3c2)c1 ; c1ccc(-c2ccc(Nc3cc(-c4ccccc4)c4oc5c(-c6ccccc6)cccc5c4c3)cc2)cc1 ; c1ccc(-c2ccc(N(c3cccc(-c4cc(-c5ccccc5)c5oc6c(-c7ccccc7)cccc6c5c4)c3)c3cc(-c4ccccc4)c4oc5c(-c6ccccc6)cccc5c4c3)cc2)cc1]

150 mL of xylene was added to Intermediate 76-4 (10.0 g, 0.023 mol), Intermediate 124-1 (17.0 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 13.8 g (yield 67.4%) of <Compound 124>.

LC/MS: m/z=881 [(M)⁺]

### Synthesis Example 18: Synthesis of Compound 146

### (1) Preparation Example 1: Synthesis of Intermediate 146-1

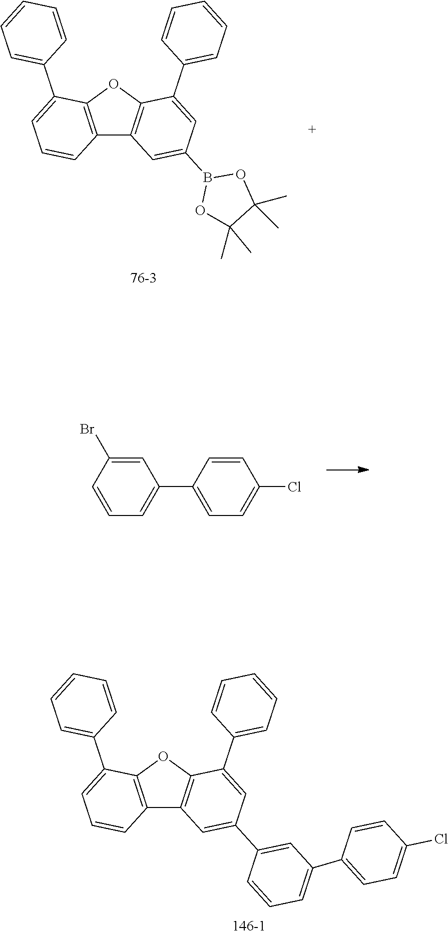 [SMILES: CC1(C)OB(c2cc(-c3ccccc3)c3oc4c(-c5ccccc5)cccc4c3c2)OC1(C)C ; Clc1ccc(-c2cccc(Br)c2)cc1 ; Clc1ccc(-c2cccc(-c3cc(-c4ccccc4)c4oc5c(-c6ccccc6)cccc5c4c3)c2)cc1]

200 mL of toluene, 50 mL of ethanol, and 50 mL of H₂O were added to Intermediate 76-3 (10.0 g, 0.022 mol), 3-bromo-4′-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.9 g (yield 60.7%) of <Intermediate 146-1>.

### (2) Preparation Example 2: Synthesis of Compound 146

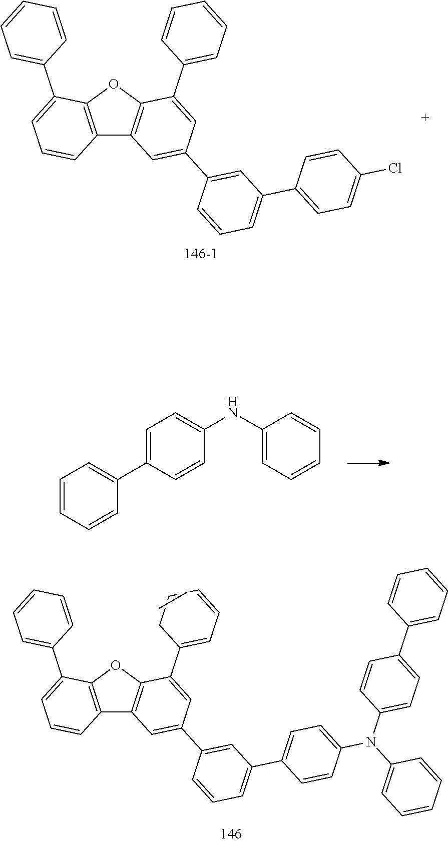

150 mL of xylene was added to Intermediate 146-1 (10.0 g, 0.020 mol), N-phenyl[1,1′-biphenyl]-4-amine (7.3 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 10.1 g (yield 71.5%) of <Compound 146>.

LC/MS: m/z=715 [(M)⁺]

### Synthesis Example 19: Synthesis of Compound 147

### (1) Preparation Example 1: Synthesis of Compound 147

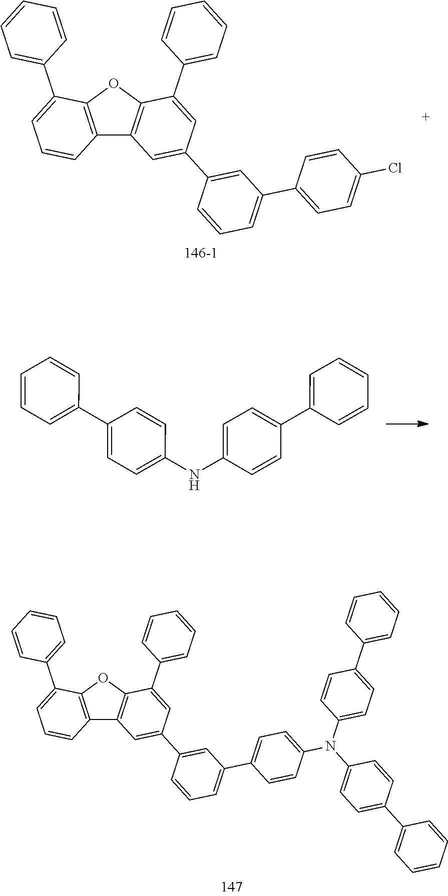 [SMILES: Clc1ccc(-c2cccc(-c3cc(-c4ccccc4)c4oc5c(-c6ccccc6)cccc5c4c3)c2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4cccc(-c5cc(-c6ccccc6)c6oc7c(-c8ccccc8)cccc7c6c5)c4)cc3)cc2)cc1]

150 mL of xylene was added to Intermediate 146-1 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 9.6 g (yield 61.5%) of <Compound 147>.

LC/MS: m/z=791 [(M)⁺]

### Synthesis Example 20: Synthesis of Compound 162

### (1) Preparation Example 1: Synthesis of Compound 162

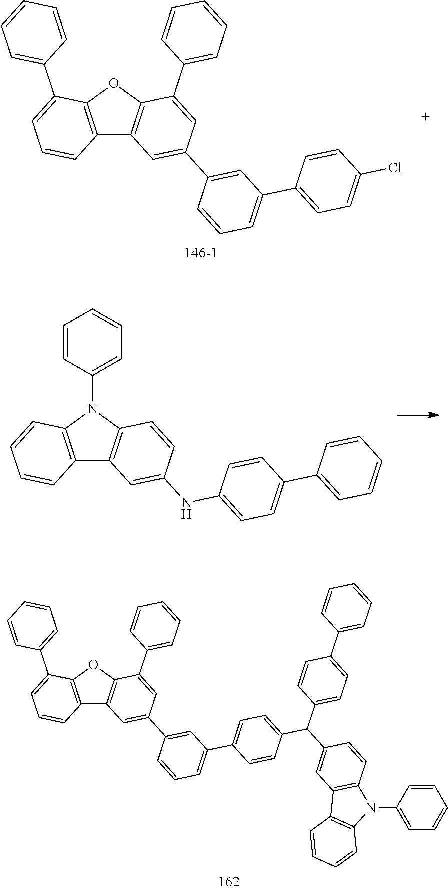 [SMILES: Clc1ccc(-c2cccc(-c3cc(-c4ccccc4)c4oc5c(-c6ccccc6)cccc5c4c3)c2)cc1 ; c1ccc(-c2ccc(Nc3ccc4c(c3)c3ccccc3n4-c3ccccc3)cc2)cc1 ; c1ccc(-c2ccc(C(c3ccc(-c4cccc(-c5cc(-c6ccccc6)c6oc7c(-c8ccccc8)cccc7c6c5)c4)cc3)c3ccc4c(c3)c3ccccc3n4-c3ccccc3)cc2)cc1]

150 mL of xylene was added to Intermediate 146-1 (10.0 g, 0.020 mol), N-[1,1′-biphenyl]-4-yl-9-phenyl-9H-carbazol-3-amine (12.1 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 11.7 g (yield 67.3%) of <Compound 162>.

LC/MS: m/z=881 [(M)⁺]

### Synthesis Example 21: Synthesis of Compound 193

### (1) Preparation Example 1: Synthesis of Intermediate 193-1

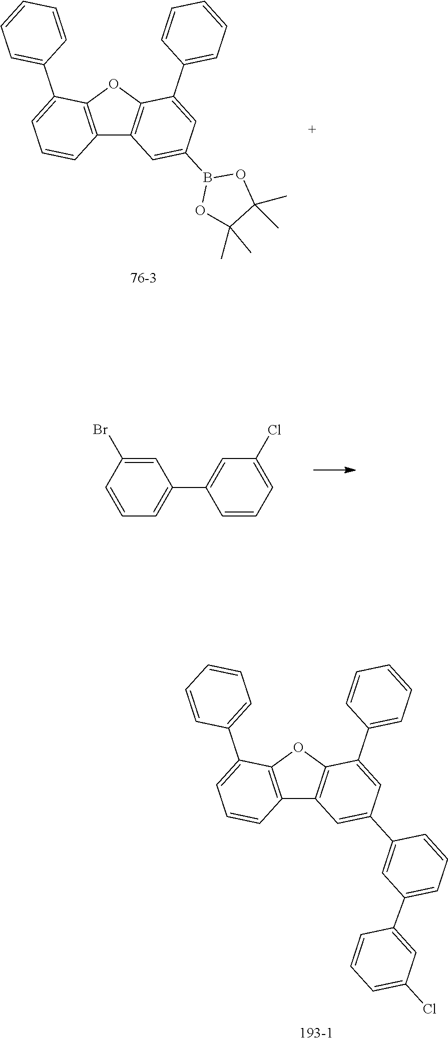 [SMILES: CC1(C)OB(c2cc(-c3ccccc3)c3oc4c(-c5ccccc5)cccc4c3c2)OC1(C)C ; Clc1cccc(-c2cccc(Br)c2)c1 ; Clc1cccc(-c2cccc(-c3cc(-c4ccccc4)c4oc5c(-c6ccccc6)cccc5c4c3)c2)c1]

200 mL of toluene, 50 mL of ethanol, and 50 mL of H₂O were added to Intermediate 76-3 (10.0 g, 0.022 mol), 3-bromo-3′-chloro-1,1′-biphenyl (7.2 g, 0.027 mol), K₂CO₃ (9.3 g, 0.067 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 7.4 g (yield 65.1%) of <Intermediate 193-1>.

### (2) Preparation Example 2: Synthesis of Compound 193

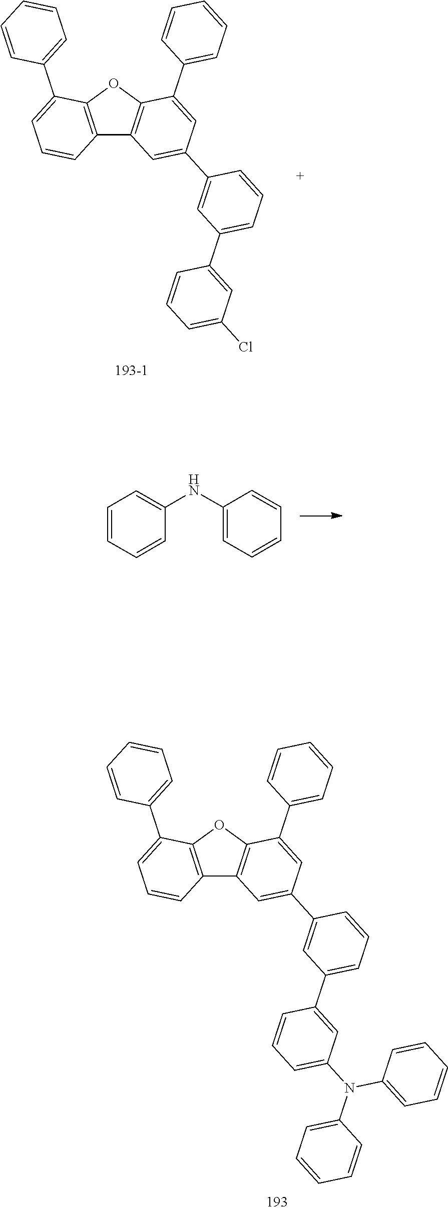 [SMILES: Clc1cccc(-c2cccc(-c3cc(-c4ccccc4)c4oc5c(-c6ccccc6)cccc5c4c3)c2)c1 ; c1ccc(Nc2ccccc2)cc1 ; c1ccc(-c2cccc3c2oc2c(-c4ccccc4)cc(-c4cccc(-c5cccc(N(c6ccccc6)c6ccccc6)c5)c4)cc23)cc1]

150 mL of xylene was added to Intermediate 193-1 (10.0 g, 0.020 mol), diphenylamine (5.0 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 7.6 g (yield 60.2%) of <Compound 193>.

LC/MS: m/z=639 [(M)⁺]

### Synthesis Example 22: Synthesis of Compound 195

### (1) Preparation Example 1: Synthesis of Intermediate 195-1

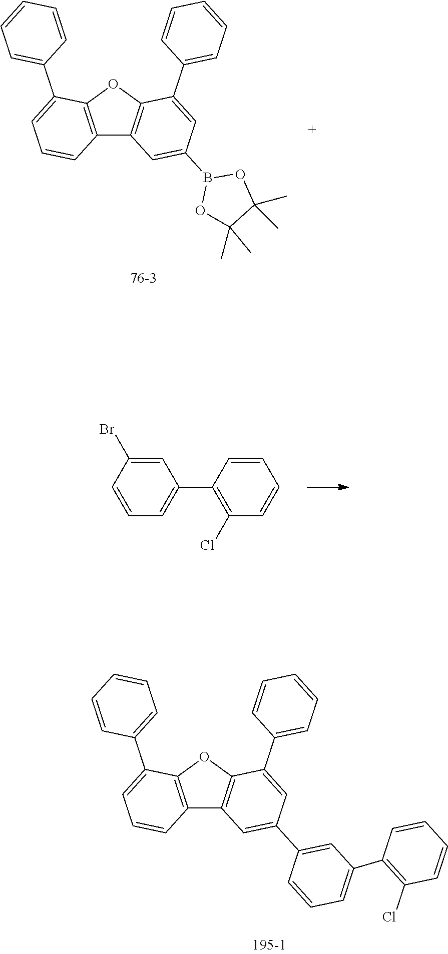 [SMILES: CC1(C)OB(c2cc(-c3ccccc3)c3oc4c(-c5ccccc5)cccc4c3c2)OC1(C)C ; Clc1ccccc1-c1cccc(Br)c1 ; Clc1ccccc1-c1cccc(-c2cc(-c3ccccc3)c3oc4c(-c5ccccc5)cccc4c3c2)c1]

200 mL of toluene, 50 mL of ethanol, and 50 mL of H₂O were added to Intermediate 76-3 (10.0 g, 0.022 mol), 3′-bromo-2-chloro-1,1′-biphenyl (7.2 g, 0.027 mol), K₂CO₃ (9.3 g, 0.067 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 7.0 g (yield 61.6%) of <Intermediate 195-1>.

### (2) Preparation Example 2: Synthesis of Compound 195

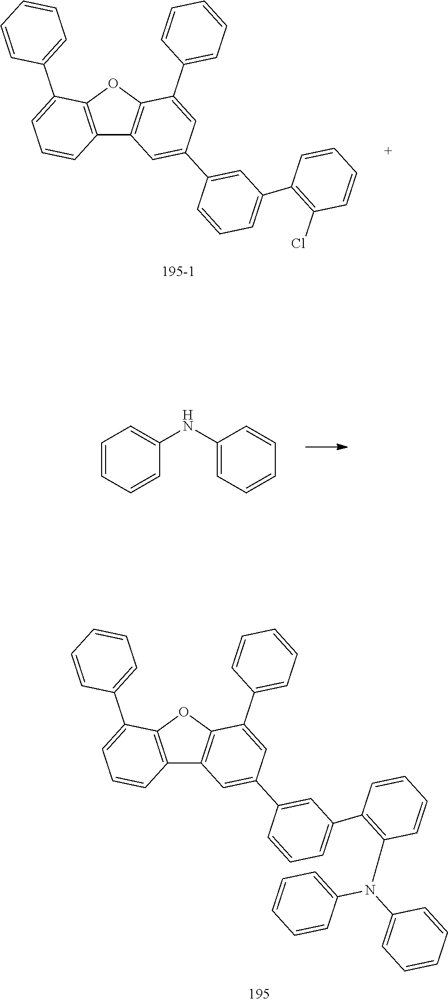 [SMILES: Clc1ccccc1-c1cccc(-c2cc(-c3ccccc3)c3oc4c(-c5ccccc5)cccc4c3c2)c1 ; c1ccc(Nc2ccccc2)cc1 ; c1ccc(-c2cccc3c2oc2c(-c4ccccc4)cc(-c4cccc(-c5ccccc5N(c5ccccc5)c5ccccc5)c4)cc23)cc1]

150 mL of xylene was added to Intermediate 195-1 (10.0 g, 0.020 mol), diphenylamine (5.0 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 7.1 g (yield 56.3%) of <Compound 195>.

LC/MS: m/z=639 [(M)⁺]

### Synthesis Example 23: Synthesis of Compound 244

### (1) Preparation Example 1: Synthesis of Intermediate 244-1

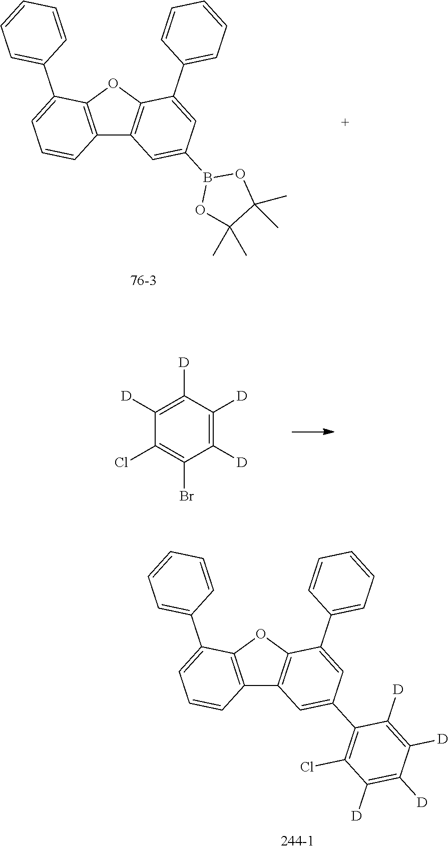 [SMILES: CC1(C)OB(c2cc(-c3ccccc3)c3oc4c(-c5ccccc5)cccc4c3c2)OC1(C)C ; [2H]c1c([2H])c([2H])c(Br)c(Cl)c1[2H] ; [2H]c1c([2H])c([2H])c(-c2cc(-c3ccccc3)c3oc4c(-c5ccccc5)cccc4c3c2)c(Cl)c1[2H]]

200 mL of toluene, 50 mL of ethanol, and 50 mL of H₂O were added to Intermediate 76-3 (10.0 g, 0.022 mol), 1-chloro-2-bromobenzene-3,4,5,6-d4 (5.3 g, 0.027 mol), K₂CO₃ (9.3 g, 0.067 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 6.7 g (yield 68.8%) of <Intermediate 244-1>.

### (2) Preparation Example 2: Synthesis of Compound 244

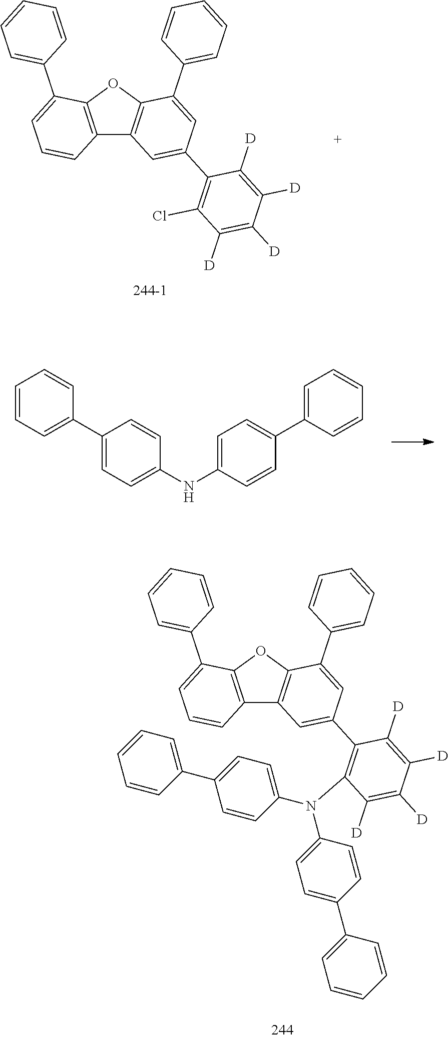 [SMILES: [2H]c1c([2H])c([2H])c(-c2cc(-c3ccccc3)c3oc4c(-c5ccccc5)cccc4c3c2)c(Cl)c1[2H] ; c1ccc(-c2ccc(Nc3ccc(-c4ccccc4)cc3)cc2)cc1 ; [2H]c1c([2H])c([2H])c(N(c2ccc(-c3ccccc3)cc2)c2ccc(-c3ccccc3)cc2)c(-c2cc(-c3ccccc3)c3oc4c(-c5ccccc5)cccc4c3c2)c1[2H]]

150 mL of xylene was added to Intermediate 244-1 (10.0 g, 0.023 mol), bis(4-biphenylyl)amine (11.1 g, 0.035 mol), NaOtBu (6.6 g, 0.069 mol), Pd(dba)₂ (0.5 g, 0.9 mmol), and t-Bu₃P (0.4 g, 1.8 mmol), and the resulting mixture was reacted under stirring at 120° C. for 4 hours. After completion of the reaction, the resulting product was extracted and concentrated, and then subjected to column chromatography and recrystallization to obtain 9.5 g (yield 57.4%) of <Compound 244>

LC/MS: m/z=719 [(M)⁺]

### Synthesis Example 24: Synthesis of Compound 259

### (1) Preparation Example 1: Synthesis of Intermediate 259-1

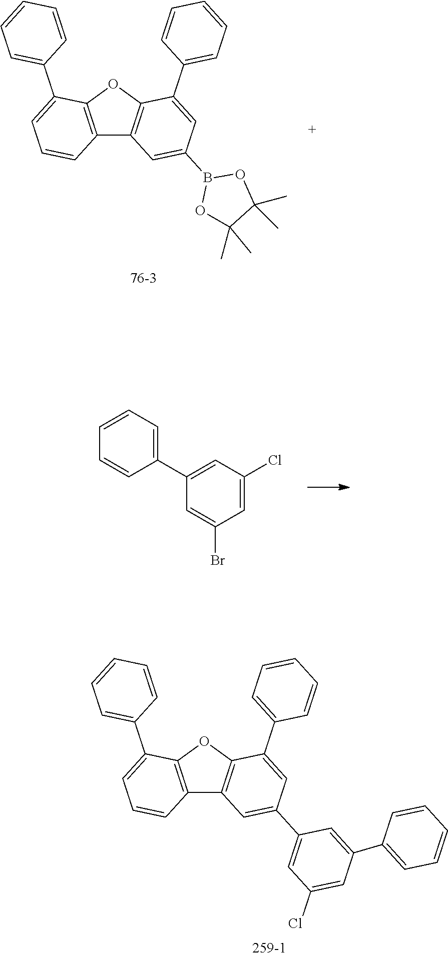 [SMILES: CC1(C)OB(c2cc(-c3ccccc3)c3oc4c(-c5ccccc5)cccc4c3c2)OC1(C)C ; Clc1cc(Br)cc(-c2ccccc2)c1 ; Clc1cc(-c2ccccc2)cc(-c2cc(-c3ccccc3)c3oc4c(-c5ccccc5)cccc4c3c2)c1]

200 mL of toluene, 50 mL of ethanol, and 50 mL of H₂O were added to Intermediate 76-3 (10.0 g, 0.022 mol), 3-bromo-5-chloro-1,1′-biphenyl (7.2 g, 0.027 mol), K₂CO₃ (9.3 g, 0.067 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 8.5 g (yield 74.8%) of <Intermediate 259-1>.

### (2) Preparation Example 2: Synthesis of Synthesis Example 259

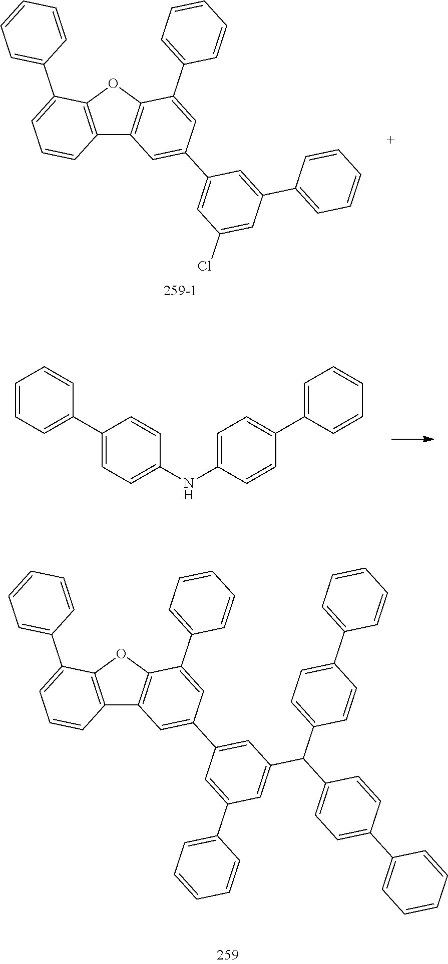 [SMILES: Clc1cc(-c2ccccc2)cc(-c2cc(-c3ccccc3)c3oc4c(-c5ccccc5)cccc4c3c2)c1 ; c1ccc(-c2ccc(Nc3ccc(-c4ccccc4)cc3)cc2)cc1 ; c1ccc(-c2ccc(C(c3ccc(-c4ccccc4)cc3)c3cc(-c4ccccc4)cc(-c4cc(-c5ccccc5)c5oc6c(-c7ccccc7)cccc6c5c4)c3)cc2)cc1]

150 mL of xylene was added to Intermediate 259-1 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 11.6 g (yield 74.3%) of <Compound 259>.

LC/MS: m/z=791 [(M)⁺]

### Synthesis Example 25: Synthesis of Compound 262

### (1) Preparation Example 1: Synthesis of Intermediate 262-1

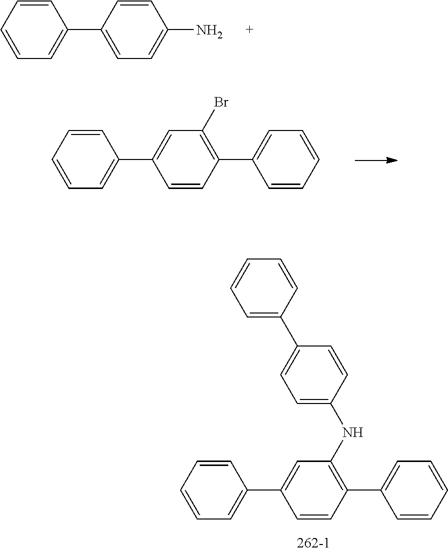 [SMILES: Nc1ccc(-c2ccccc2)cc1 ; "+" ; Brc1cc(-c2ccccc2)ccc1-c1ccccc1 ; c1ccc(-c2ccc(Nc3cc(-c4ccccc4)ccc3-c3ccccc3)cc2)cc1]

150 mL of xylene was added to 4-aminobiphenyl (10.0 g, 0.059 mol), 2′-bromo-1,1′: 4′,1″-terphenyl (27.4 g, 0.089 mol), NaOtBu (17.0 g, 0.177 mol), Pd(dba)₂ (1.4 g, 2.4 mmol), and t-Bu₃P (1.0 g, 4.7 mmol), and the resulting mixture was reacted under stirring at 120° C. for 4 hours. After completion of the reaction, the resulting product was extracted and concentrated, and then subjected to column chromatography and recrystallization to obtain 10.8 g (yield 46.0%) of <Intermediate 262-1>.

### (2) Preparation Example 2: Synthesis of Compound 262

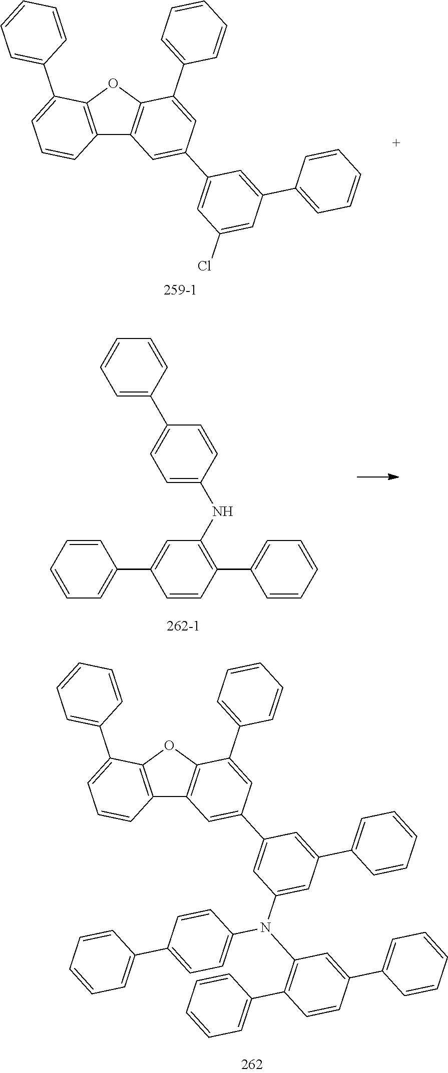 [SMILES: Clc1cc(-c2ccccc2)cc(-c2cc(-c3ccccc3)c3oc4c(-c5ccccc5)cccc4c3c2)c1 ; c1ccc(-c2ccc(Nc3cc(-c4ccccc4)ccc3-c3ccccc3)cc2)cc1 ; c1ccc(-c2ccc(N(c3cc(-c4ccccc4)cc(-c4cc(-c5ccccc5)c5oc6c(-c7ccccc7)cccc6c5c4)c3)c3cc(-c4ccccc4)ccc3-c3ccccc3)cc2)cc1]

150 mL of xylene was added to Intermediate 259-1 (10.0 g, 0.020 mol), Intermediate 262-1 (11.8 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 10.1 g (yield 59.0%) of <Compound 262>.

LC/MS: m/z=867 [(M)⁺]

### Synthesis Example 26: Synthesis of Compound 270

### (1) Preparation Example 1: Synthesis of Compound 270

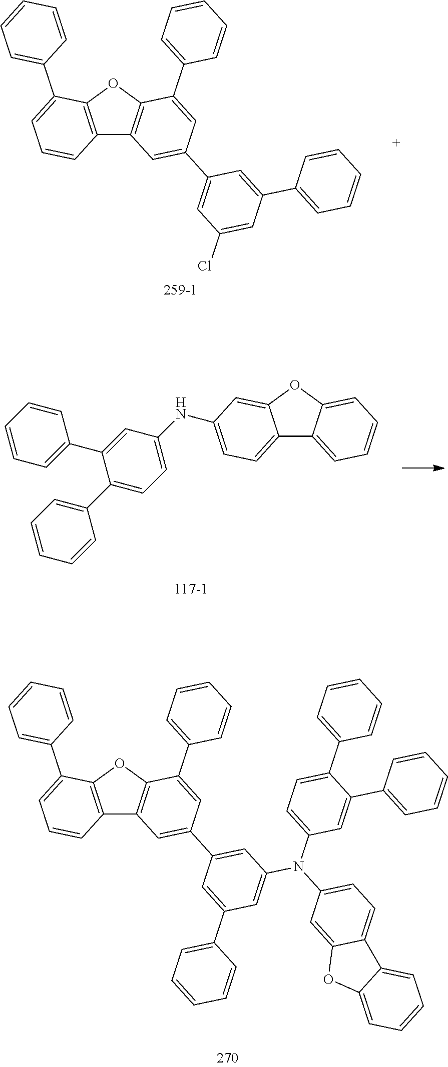 [SMILES: Clc1cc(-c2ccccc2)cc(-c2cc(-c3ccccc3)c3oc4c(-c5ccccc5)cccc4c3c2)c1 ; c1ccc(-c2ccc(Nc3ccc4c(c3)oc3ccccc34)cc2-c2ccccc2)cc1 ; c1ccc(-c2cc(-c3cc(-c4ccccc4)c4oc5c(-c6ccccc6)cccc5c4c3)cc(N(c3ccc(-c4ccccc4)c(-c4ccccc4)c3)c3ccc4c(c3)oc3ccccc34)c2)cc1]

150 mL of xylene was added to Intermediate 259-1 (10.0 g, 0.020 mol), Intermediate 117-1 (12.2 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 9.2 g (yield 52.9%) of <Compound 270>.

LC/MS: m/z=881 [(M)⁺]

### Synthesis Example 27: Synthesis of Compound 277

### (1) Preparation Example 1: Synthesis of Compound 277

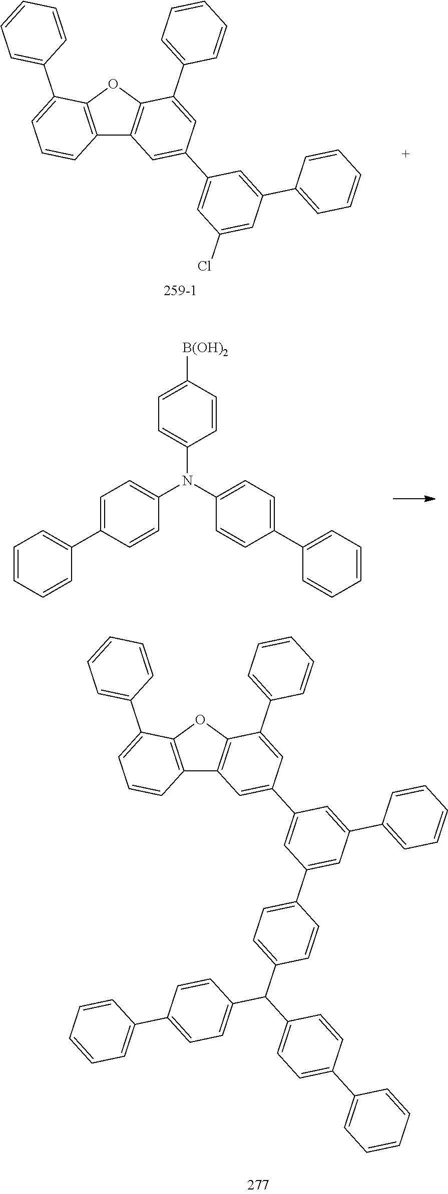 [SMILES: Clc1cc(-c2ccccc2)cc(-c2cc(-c3ccccc3)c3oc4c(-c5ccccc5)cccc4c3c2)c1 ; OB(O)c1ccc(N(c2ccc(-c3ccccc3)cc2)c2ccc(-c3ccccc3)cc2)cc1 ; c1ccc(-c2ccc(C(c3ccc(-c4ccccc4)cc3)c3ccc(-c4cc(-c5ccccc5)cc(-c5cc(-c6ccccc6)c6oc7c(-c8ccccc8)cccc7c6c5)c4)cc3)cc2)cc1]

Intermediate 259-1 (10.0 g, 0.020 mol), B-[4-[bis([1,1′-biphenyl]-4-yl)amino]phenyl]boronic acid (10.5 g, 0.024 mol), K₂CO₃ (8.2 g, 0.059 mol), Pd(OAc)₂ (1.1 g, 1.0 mmol), X-Phos (0.9 g, 2.0 mmol), 200 mL of THF, and 50 mL of H₂O were put into a container, and the resulting mixture was reacted under stirring at 120° C. for 6 hours. After completion of the reaction, the resulting product was extracted and concentrated, and then subjected to column chromatography and recrystallization to obtain 9.5 g (yield 55.5%) of <Compound 277>.

LC/MS: m/z=868 [(M)⁺]

### Synthesis Example 28: Synthesis of Compound 294

### (1) Preparation Example 1: Synthesis of Intermediate 294-1

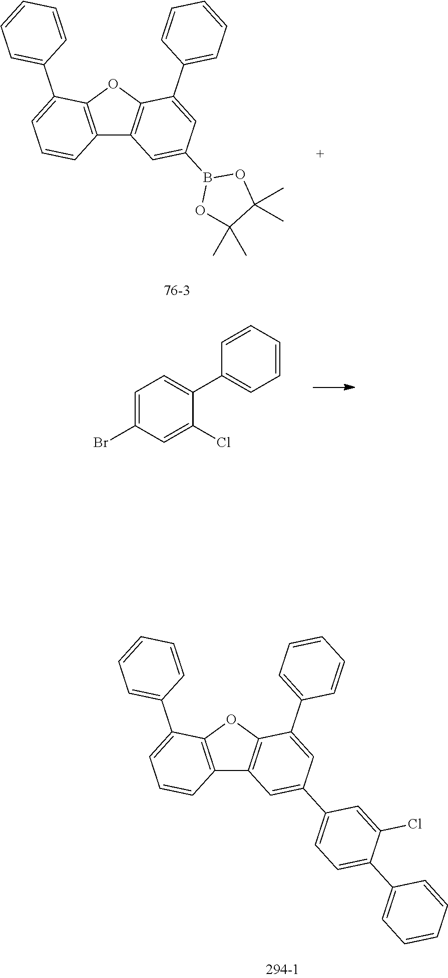 [SMILES: CC1(C)OB(c2cc(-c3ccccc3)c3oc4c(-c5ccccc5)cccc4c3c2)OC1(C)C ; Clc1cc(Br)ccc1-c1ccccc1 ; Clc1cc(-c2cc(-c3ccccc3)c3oc4c(-c5ccccc5)cccc4c3c2)ccc1-c1ccccc1]

200 mL of toluene, 50 mL of ethanol, and 50 mL of H₂O were added to Intermediate 76-3 (10.0 g, 0.022 mol), 4-bromo-2-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.5 g (yield 57.2%) of <Intermediate 294-1>.

(2) Preparation Example 2: Synthesis of Compound 294

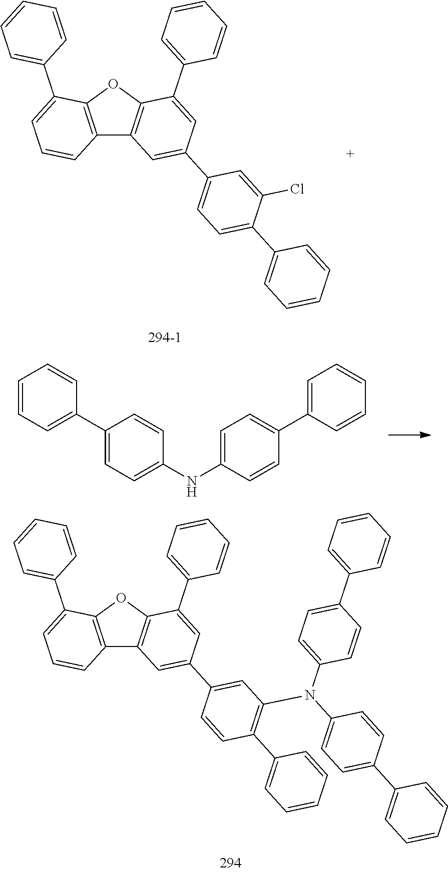

150 mL of xylene was added to Intermediate 294-1 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 11.2 g (yield 71.7%) of <Compound 294>.

LC/MS: m/z=791 [(M)⁺]

### Synthesis Example 29: Synthesis of Compound 338

### (1) Preparation Example 1: Synthesis of Intermediate 338-1

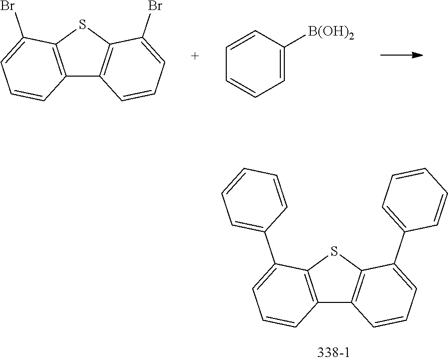 [SMILES: Brc1cccc2c1sc1c(Br)cccc12 ; OB(O)c1ccccc1 ; c1ccc(-c2cccc3c2sc2c(-c4ccccc4)cccc23)cc1]

200 mL of toluene, 50 mL of ethanol, and 50 mL of H₂O were added to 4,6-dibromodibenzothiophene (10.0 g, 0.029 mol), phenylboronic acid (8.6 g, 0.070 mol), K₂CO₃ (24.2 g, 0.175 mol), and Pd(PPh₃)₄ (0.7 g, 0.6 mmol), and the resulting mixture was reacted under stirring at 80° C. for 6 hours. After completion of the reaction, the resulting product was extracted and concentrated, and then subjected to column chromatography to obtain 6.4 g (yield 65.1%) of <Intermediate 338-1>.

### (2) Preparation Example 2: Synthesis of Intermediate 338-2

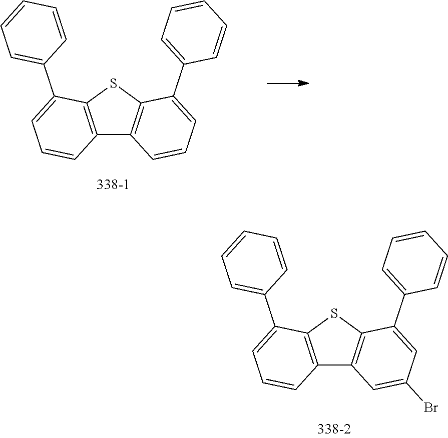 [SMILES: c1ccc(-c2cccc3c2sc2c(-c4ccccc4)cccc23)cc1 ; Brc1cc(-c2ccccc2)c2sc3c(-c4ccccc4)cccc3c2c1]

After Intermediate 338-1 was dissolved in 100 mL of CHCl₃, bromine (in CHCl₃, 11.9 g, 0.074 mol) was slowly added dropwise thereto, and the resulting mixture was reacted under stirring at room temperature for 12 hours. After completion of the reaction, the resulting product was extracted and concentrated, and then subjected to column chromatography to obtain 5.1 g (yield 41.3%) of <Intermediate 338-2> (3) Preparation Example 3: Synthesis of Intermediate 338-3

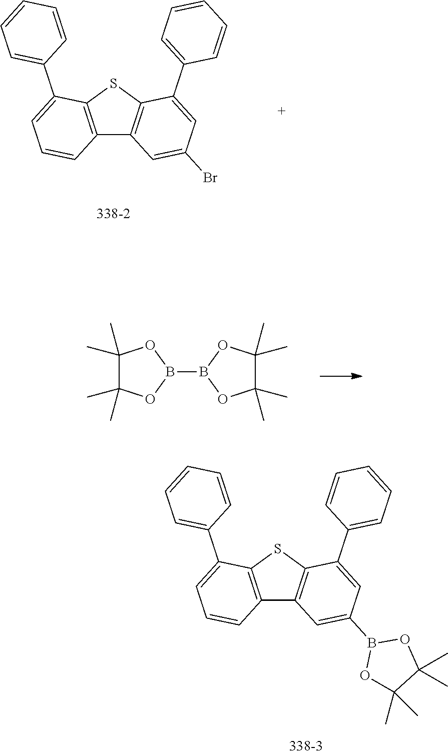

200 mL of dioxane was added to Intermediate 338-2 (10.0 g, 0.024 mol), bis(pinacolato)diboron (7.3 g, 0.029 mol), KOAc (7.1 g, 0.072 mol), and Pd(dppf)Cl₂ (0.9 g, 0.001 mol), and the resulting mixture was reacted under stirring at 100° C. for 12 hours. After completion of the reaction, the resulting product was extracted and concentrated, and then subjected to column chromatography to obtain 7.7 g (yield 69.2%) of <Intermediate 338-3>.

### (4) Preparation Example 4: Synthesis of Intermediate 338-4

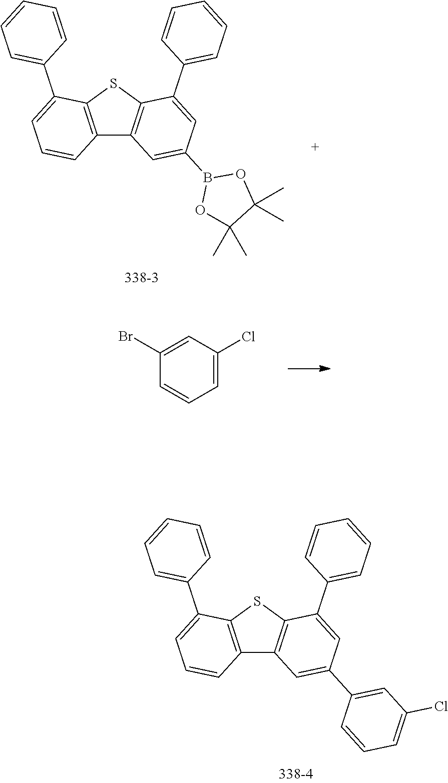 [SMILES: CC1(C)OB(c2cc(-c3ccccc3)c3sc4c(-c5ccccc5)cccc4c3c2)OC1(C)C ; Clc1cccc(Br)c1 ; Clc1cccc(-c2cc(-c3ccccc3)c3sc4c(-c5ccccc5)cccc4c3c2)c1]

200 mL of toluene, 50 mL of ethanol, and 50 mL of H₂O were added to Intermediate 338-3 (10.0 g, 0.022 mol), 1-bromo-3-chlorobenzene (5.0 g, 0.026 mol), K₂CO₃ (9.0 g, 0.065 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 6.2 g (yield 64.2%) of <Intermediate 338-4>.

### (5) Preparation Example 5: Synthesis of Compound 338

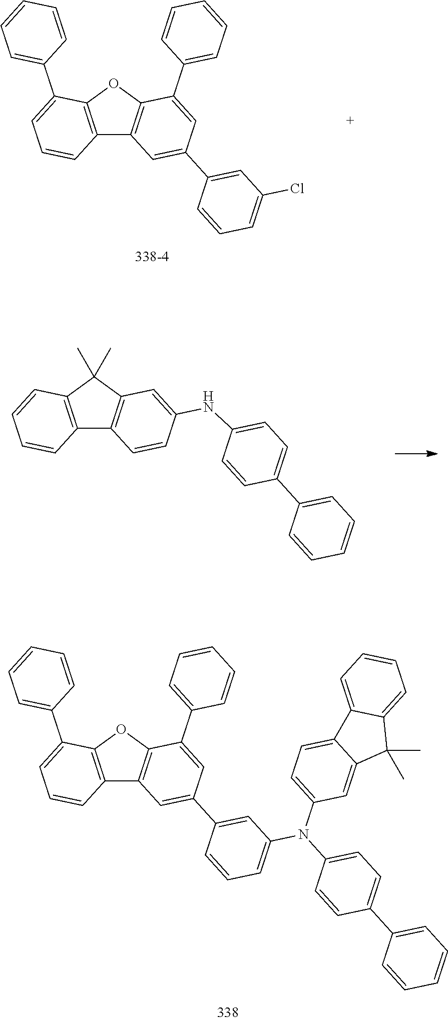 [SMILES: Clc1cccc(-c2cc(-c3ccccc3)c3oc4c(-c5ccccc5)cccc4c3c2)c1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3cccc(-c4cc(-c5ccccc5)c5oc6c(-c7ccccc7)cccc6c5c4)c3)cc21]

150 mL of xylene was added to Intermediate 338-4 (10.0 g, 0.022 mol), N-[1,1′-biphenyl]-4-yl-9,9-dimethyl-9H-fluoren-2-amine (12.1 g, 0.034 mol), NaOtBu (6.5 g, 0.067 mol), Pd(dba)₂ (0.5 g, 0.9 mmol), and t-Bu₃P (0.4 g, 1.8 mmol), and the resulting mixture was reacted under stirring at 120° C. for 4 hours. After completion of the reaction, the resulting product was extracted and concentrated, and then subjected to column chromatography and recrystallization to obtain 12.4 g (yield 71.8%) of <Compound 338>.

LC/MS: m/z=771 [(M)⁺]

### Synthesis Example 30: Synthesis of Compound 348

### (1) Preparation Example 1: Synthesis of Intermediate 348-1

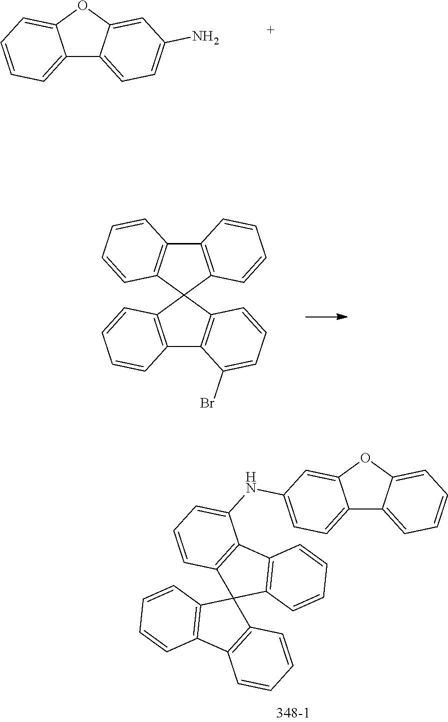 [SMILES: Nc1ccc2c(c1)oc1ccccc12 ; Brc1cccc2c1-c1ccccc1C21c2ccccc2-c2ccccc21 ; c1ccc2c(c1)-c1ccccc1C21c2ccccc2-c2c(Nc3ccc4c(c3)oc3ccccc34)cccc21]

150 mL of xylene was added to dibenzo[b,d]furan-3-amine (10.0 g, 0.055 mol), 4-bromo-9,9′-spirobi[9H-fluorene] (32.4 g, 0.082 mol), NaOtBu (15.7 g, 0.164 mol), Pd(dba)₂ (1.3 g, 2.2 mmol), and t-Bu₃P (0.9 g, 4.4 mmol), and the resulting mixture was reacted under stirring at 120° C. for 4 hours. After completion of the reaction, the resulting product was extracted and concentrated, and then subjected to column chromatography and recrystallization to obtain 11.8 g (yield 43.5%) of <Intermediate 348-1>.

### (2) Preparation Example 2: Synthesis of Compound 348

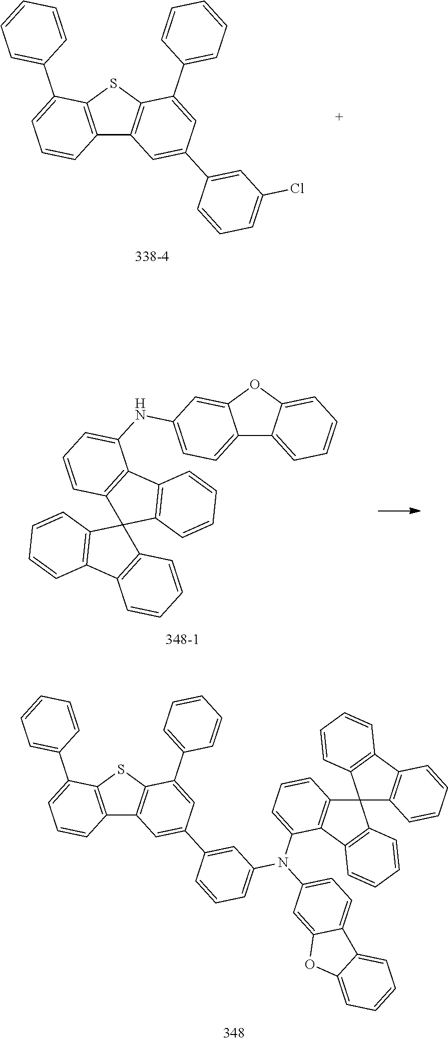 [SMILES: Clc1cccc(-c2cc(-c3ccccc3)c3sc4c(-c5ccccc5)cccc4c3c2)c1 ; c1ccc2c(c1)-c1ccccc1C21c2ccccc2-c2c(Nc3ccc4c(c3)oc3ccccc34)cccc21 ; c1ccc(-c2cccc3c2sc2c(-c4ccccc4)cc(-c4cccc(N(c5ccc6c(c5)oc5ccccc56)c5cccc6c5-c5ccccc5C65c6ccccc6-c6ccccc65)c4)cc23)cc1]

150 mL of xylene was added to Intermediate 338-4 (10.0 g, 0.022 mol), Intermediate 348-1 (16.7 g, 0.034 mol), NaOtBu (6.5 g, 0.067 mol), Pd(dba)₂ (0.5 g, 0.9 mmol), and t-Bu₃P (0.4 g, 1.8 mmol), and the resulting mixture was reacted under stirring at 120° C. for 4 hours. After completion of the reaction, the resulting product was extracted and concentrated, and then subjected to column chromatography and recrystallization to obtain 11.2 g (yield 55.1%) of <Compound 348>.

LC/MS: m/z=907 [(M)⁺]

### Synthesis Example 31: Synthesis of Compound 410

### (1) Preparation Example 1: Synthesis of Intermediate 410-1

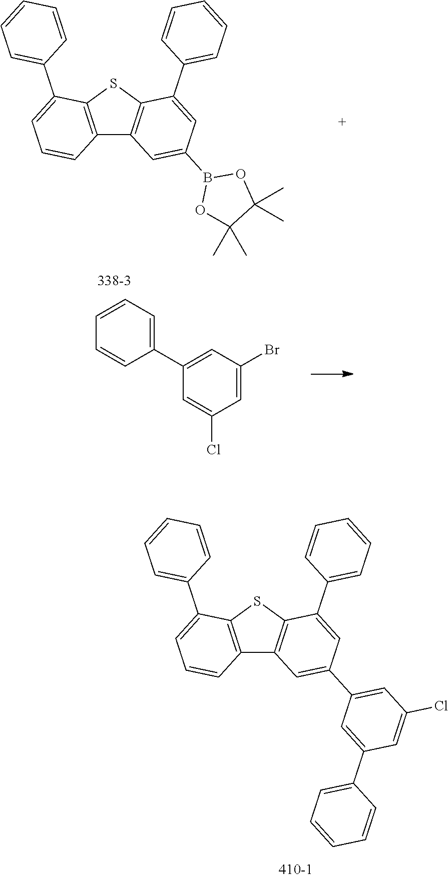 [SMILES: CC1(C)OB(c2cc(-c3ccccc3)c3sc4c(-c5ccccc5)cccc4c3c2)OC1(C)C ; Clc1cc(Br)cc(-c2ccccc2)c1 ; Clc1cc(-c2ccccc2)cc(-c2cc(-c3ccccc3)c3sc4c(-c5ccccc5)cccc4c3c2)c1]

200 mL of toluene, 50 mL of ethanol, and 50 mL of H₂O were added to Intermediate 338-3 (10.0 g, 0.022 mol), 3-bromo-5-chloro-1,1′-biphenyl (6.9 g, 0.026 mol), K₂CO₃ (9.0 g, 0.065 mol), and Pd(PPh₃)₄ (0.5 g, 0.0004 mol), and the resulting mixture was reacted under stirring at 80° C. for 6 hours. After completion of the reaction, the resulting product was extracted and concentrated, and then subjected to column chromatography and recrystallization to obtain 8.1 g (yield 71.6%) of <Intermediate 410-1>.

### (2) Preparation Example 2: Synthesis of Compound 410

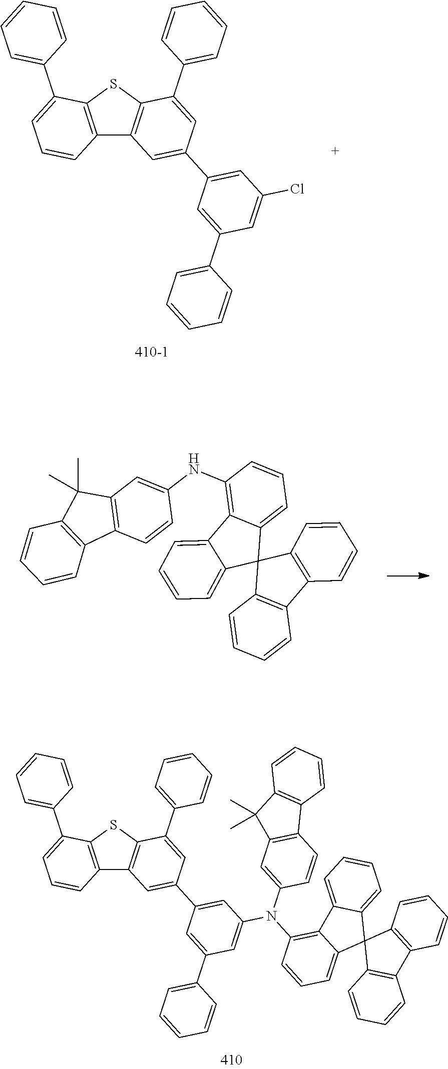 [SMILES: Clc1cc(-c2ccccc2)cc(-c2cc(-c3ccccc3)c3sc4c(-c5ccccc5)cccc4c3c2)c1 ; CC1(C)c2ccccc2-c2ccc(Nc3cccc4c3-c3ccccc3C43c4ccccc4-c4ccccc43)cc21 ; CC1(C)c2ccccc2-c2ccc(N(c3cc(-c4ccccc4)cc(-c4cc(-c5ccccc5)c5sc6c(-c7ccccc7)cccc6c5c4)c3)c3cccc4c3-c3ccccc3C43c4ccccc4-c4ccccc43)cc21]

150 mL of xylene was added to Intermediate 410-4 (10.0 g, 0.019 mol), N-(9,9-dimethyl-9H-fluoren-2-yl)-9,9′-spirobi[9H-fluoren]-4-amine (15.0 g, 0.029 mol), NaOtBu (5.5 g, 0.057 mol), Pd(dba)₂ (0.4 g, 0.8 mmol), and t-Bu₃P (0.3 g, 1.5 mmol), and the resulting mixture was reacted under stirring at 120° C. for 4 hours. After completion of the reaction, the resulting product was extracted and concentrated, and then subjected to column chromatography and recrystallization to obtain 9.6 g (yield 49.7%) of <Compound 410>.

LC/MS: m/z=1009 [(M)⁺]

### Synthesis Example 32: Synthesis of Compound 431

### (1) Preparation Example 1: Synthesis of Intermediate 431-1

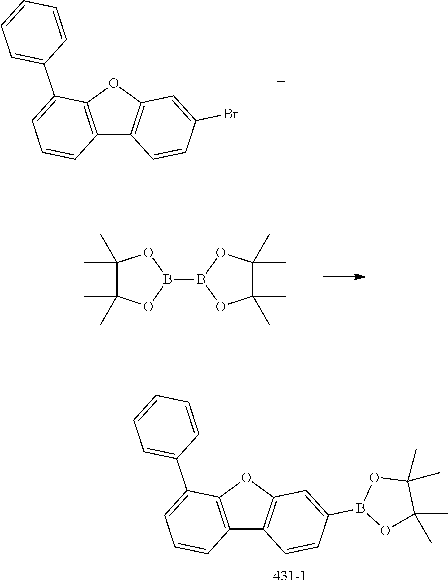

200 mL of dioxane was added to 3-bromo-6-phenyldibenzofuran (10.0 g, 0.031 mol), bis(pinacolato)diboron (9.4 g, 0.037 mol), KOAc (9.1 g, 0.093 mol), and Pd(dppf)Cl₂ (1.1 g, 1.5 mmol), and the resulting mixture was reacted under stirring at 100° C. for 12 hours. After completion of the reaction, the resulting product was extracted and concentrated, and then subjected to column chromatography and recrystallization to obtain 8.3 g (yield 72.5%) of <Intermediate 431-1>.

### (2) Preparation Example 2: Synthesis of Compound 431

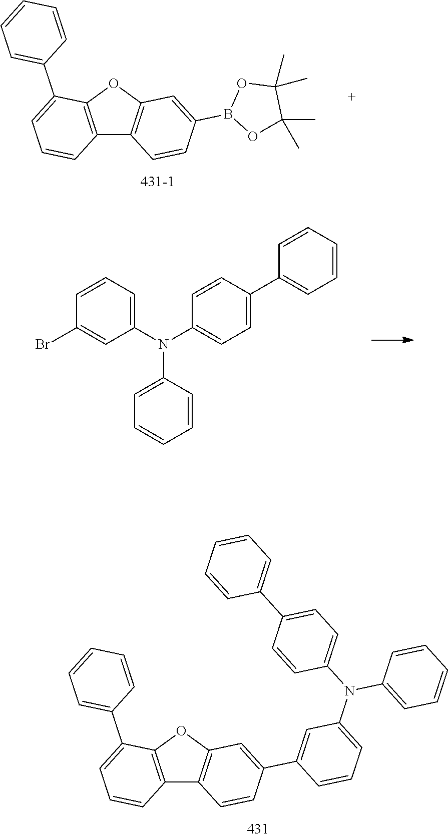 [SMILES: CC1(C)OB(c2ccc3c(c2)oc2c(-c4ccccc4)cccc23)OC1(C)C ; Brc1cccc(N(c2ccccc2)c2ccc(-c3ccccc3)cc2)c1 ; c1ccc(-c2ccc(N(c3ccccc3)c3cccc(-c4ccc5c(c4)oc4c(-c6ccccc6)cccc45)c3)cc2)cc1]

200 mL of toluene, 50 mL of ethanol, and 50 mL of H₂O were added to Intermediate 431-1 (10.0 g, 0.027 mol), N-(3-bromophenyl)-N-phenyl[1,1′-biphenyl]-4-amine (13.0 g, 0.032 mol), K₂CO₃ (11.2 g, 0.081 mol), and Pd(PPh₃)₄ (0.6 g, 0.5 mmol), and the resulting mixture was reacted under stirring at 80° C. for 6 hours. After completion of the reaction, the resulting product was extracted and concentrated, and then subjected to column chromatography and recrystallization to obtain 9.4 g (yield 61.7%) of <Compound 431>.

LC/MS: m/z=563 [(M)⁺]

### Synthesis Example 33: Synthesis of Compound 508

### (1) Preparation Example 1: Synthesis of Intermediate 508-1

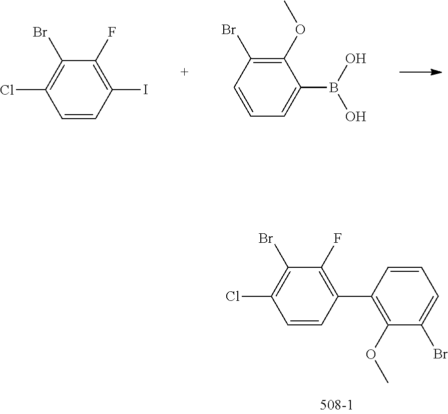 [SMILES: Fc1c(I)ccc(Cl)c1Br ; COc1c(Br)cccc1B(O)O ; COc1c(Br)cccc1-c1ccc(Cl)c(Br)c1F]

200 mL of toluene, 50 mL of ethanol, and 50 mL of H₂O were added to 2-bromo-1-chloro-3-fluoro-4-iodobenzene (10.0 g, 0.030 mol), 3-bromo-2-methoxyphenylboronic acid (8.3 g, 0.036 mol), K₂CO₃ (12.4 g, 0.090 mol), and Pd(PPh₃)₄ (0.7 g, 0.6 mmol), and the resulting mixture was reacted under stirring at 80° C. for 6 hours. After completion of the reaction, the resulting product was extracted and concentrated, and then subjected to column chromatography to obtain 4.9 g (yield 41.7%) of <Intermediate 508-1>.

### (2) Preparation Example 2: Synthesis of Intermediate 508-2

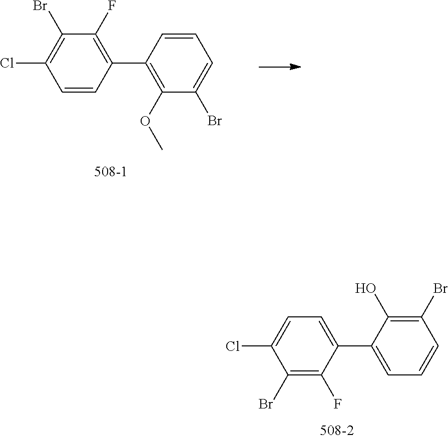 [SMILES: COc1c(Br)cccc1-c1ccc(Cl)c(Br)c1F ; Oc1c(Br)cccc1-c1ccc(Cl)c(Br)c1F]

Intermediate 1-1 (10.0 g, 0.025 mol) was dissolved in 60 mL of anhydrous DCM. Thereafter, BBr₃ (in DCM, 15.9 g, 0.063 mol) was added dropwise while maintaining the temperature at 0° C., and the resulting mixture was reacted under stirring at 25° C. for 16 hours. After completion of the reaction, the resulting product was extracted and concentrated, and then subjected to column chromatography to obtain 7.1 g (yield 73.6%) of <Intermediate 1-2>.

### (3) Preparation Example 3: Synthesis of Intermediate 508-3

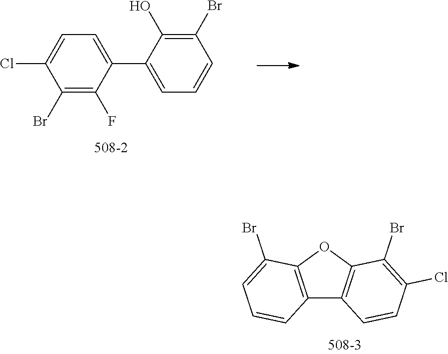 [SMILES: Oc1c(Br)cccc1-c1ccc(Cl)c(Br)c1F ; Clc1ccc2c(oc3c(Br)cccc32)c1Br]

500 mL of NMP was added to Intermediate 508-2 (10.0 g, 0.026 mol) and K₂CO₃ (22.6 g, 0.066 mol), and the resulting mixture was reacted under stirring at 180° C. for 24 hours. After completion of the reaction, the resulting product was extracted and concentrated, and then subjected to column chromatography to obtain 4.5 g (yield 47.5%) of <Intermediate 508-3>.

### (4) Preparation Example 4: Synthesis of Intermediate 508-4

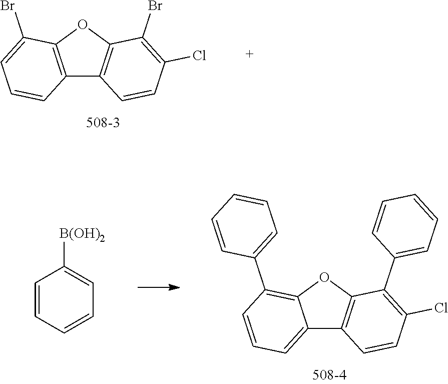 [SMILES: Clc1ccc2c(oc3c(Br)cccc32)c1Br ; OB(O)c1ccccc1 ; Clc1ccc2c(oc3c(-c4ccccc4)cccc32)c1-c1ccccc1]

200 mL of toluene, 50 mL of ethanol, and 50 mL of H₂O were added to Intermediate 508-3 (10.0 g, 0.028 mol), phenylboronic acid (8.1 g, 0.067 mol), K₂CO₃ (23.0 g, 0.167 mol), and Pd(PPh₃)₄ (0.6 g, 0.6 mmol), and the resulting mixture was reacted under stirring at 80° C. for 6 hours. After completion of the reaction, the resulting product was extracted and concentrated, and then subjected to column chromatography to obtain 6.0 g (yield 61.0%) of <Intermediate 508-4>.

### (5) Preparation Example 5: Synthesis of Intermediate 508-5

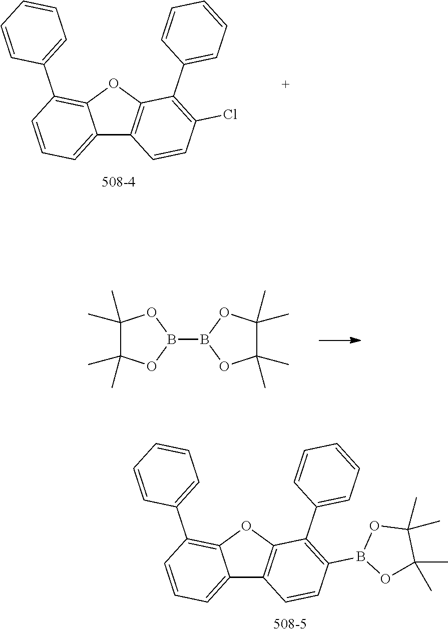

200 mL of dioxane was added to Intermediate 508-4 (10.0 g, 0.028 mol), bis(pinacolato)diboron (8.6 g, 0.034 mol), KOAc (8.3 g, 0.085 mol), and Pd(dppf)Cl₂ (1.0 g, 1.4 mmol), and the resulting mixture was reacted under stirring at 100° C. for 12 hours. After completion of the reaction, the resulting product was extracted and concentrated, and then subjected to column chromatography to obtain 8.8 g (yield 70.0%) of <Intermediate 508-5>.

### (6) Preparation Example 6: Synthesis of Intermediate 508-6

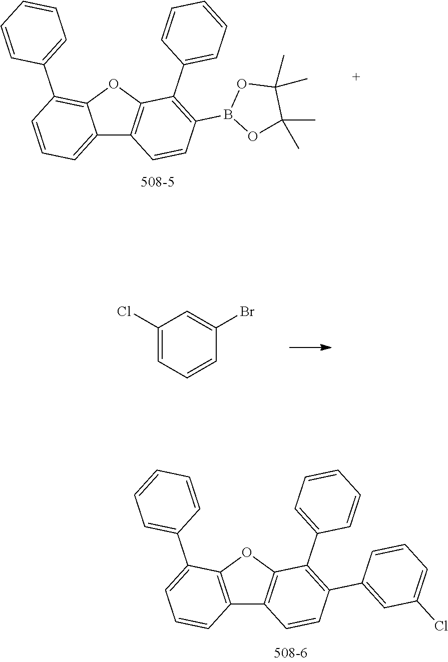 [SMILES: CC1(C)OB(c2ccc3c(oc4c(-c5ccccc5)cccc43)c2-c2ccccc2)OC1(C)C ; Clc1cccc(Br)c1 ; Clc1cccc(-c2ccc3c(oc4c(-c5ccccc5)cccc43)c2-c2ccccc2)c1]

200 mL of toluene, 50 mL of ethanol, and 50 mL of H₂O were added to Intermediate 508-5 (10.0 g, 0.022 mol), 1-bromo-3-chlorobenzene (5.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.2 g (yield 64.2%) of <Intermediate 508-6>.

### (7) Preparation Example 7: Synthesis of Compound 508

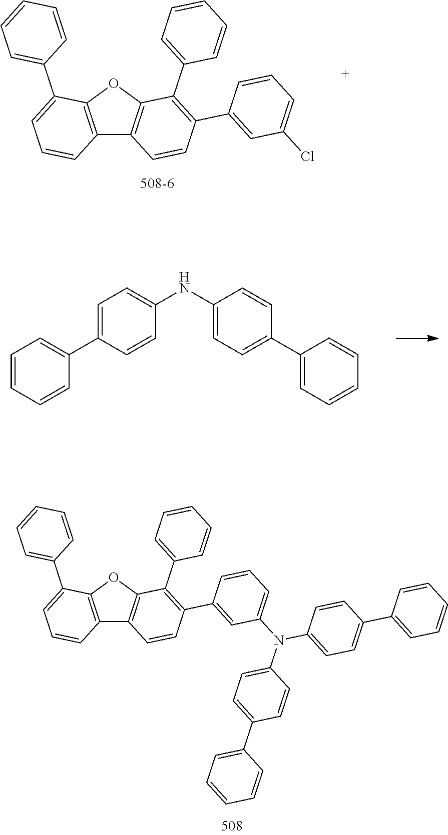 [SMILES: Clc1cccc(-c2ccc3c(oc4c(-c5ccccc5)cccc43)c2-c2ccccc2)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5c(oc6c(-c7ccccc7)cccc65)c4-c4ccccc4)c3)cc2)cc1]

150 mL of xylene was added to Intermediate 508-6 (10.0 g, 0.023 mol), bis(4-biphenylyl)amine (11.2 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 10.9 g (yield 65.6%) of <Compound 508>.

LC/MS: m/z=715 [(M)⁺]

### Synthesis Example 34: Synthesis of Compound 525

### (1) Preparation Example 1: Synthesis of Compound 525

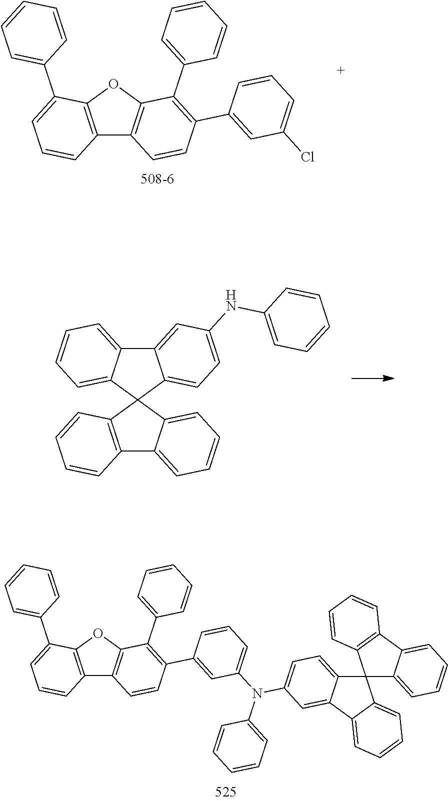 [SMILES: Clc1cccc(-c2ccc3c(oc4c(-c5ccccc5)cccc43)c2-c2ccccc2)c1 ; "+" ; c1ccc(Nc2ccc3c(c2)-c2ccccc2C32c3ccccc3-c3ccccc32)cc1 ; c1ccc(-c2cccc3c2oc2c(-c4ccccc4)c(-c4cccc(N(c5ccccc5)c5ccc6c(c5)-c5ccccc5C65c6ccccc6-c6ccccc65)c4)ccc23)cc1]

150 mL of xylene was added to Intermediate 508-6 (10.0 g, 0.023 mol), N-phenyl-9,9′-spirobi[9H-fluoren]-3-amine (14.2 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 13.2 g (yield 70.9%) of <Compound 525>.

LC/MS: m/z=801 [(M)⁺]

### Synthesis Example 35: Synthesis of Compound 539

### (1) Preparation Example 1: Synthesis of Compound 539

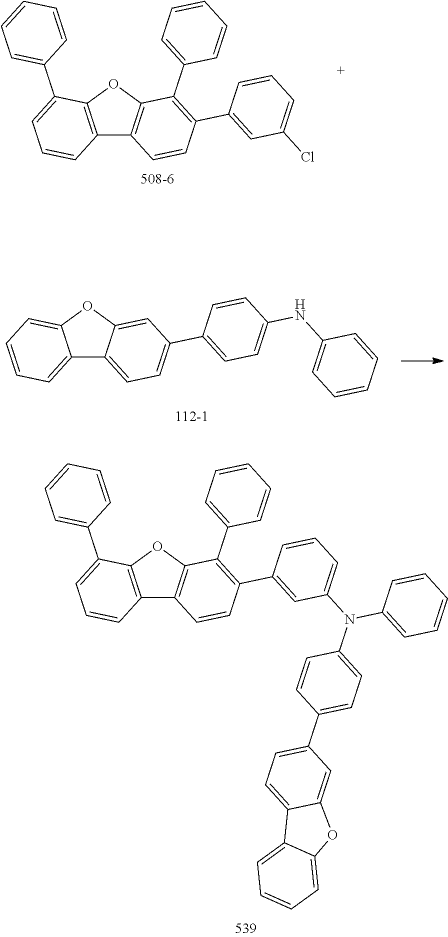 [SMILES: Clc1cccc(-c2ccc3c(oc4c(-c5ccccc5)cccc43)c2-c2ccccc2)c1 ; "+" ; c1ccc(Nc2ccc(-c3ccc4c(c3)oc3ccccc34)cc2)cc1 ; c1ccc(-c2cccc3c2oc2c(-c4ccccc4)c(-c4cccc(N(c5ccccc5)c5ccc(-c6ccc7c(c6)oc6ccccc67)cc5)c4)ccc23)cc1]

150 mL of xylene was added to Intermediate 508-6 (10.0 g, 0.023 mol), Intermediate 112-1 (11.7 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.1 g (yield 65.5%) of <Compound 539>.

LC/MS: m/z=729 [(M)⁺]

### Synthesis Example 36: Synthesis of Compound 569

### (1) Preparation Example 1: Synthesis of Intermediate 569-1

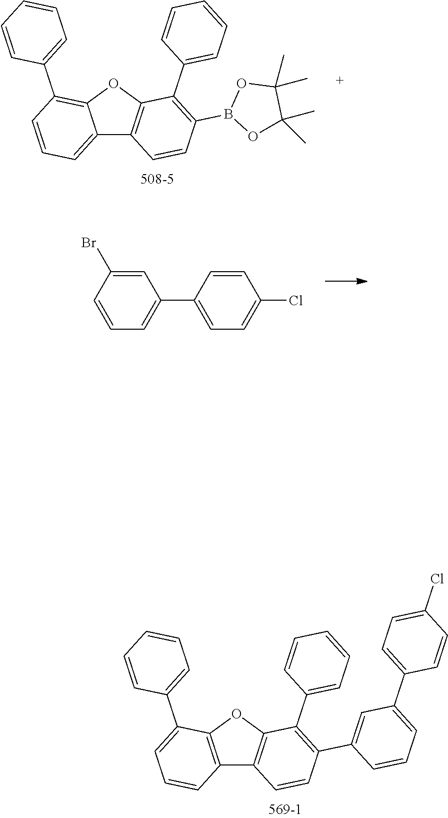 [SMILES: CC1(C)OB(c2ccc3c(oc4c(-c5ccccc5)cccc43)c2-c2ccccc2)OC1(C)C ; Clc1ccc(-c2cccc(Br)c2)cc1 ; Clc1ccc(-c2cccc(-c3ccc4c(oc5c(-c6ccccc6)cccc54)c3-c3ccccc3)c2)cc1]

200 mL of toluene, 50 mL of ethanol, and 50 mL of H₂O were added to Intermediate 508-5 (10.0 g, 0.022 mol), 3-bromo-4′-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.5 g (yield 57.2%) of <Intermediate 569-1>.

### (2) Preparation Example 2: Synthesis of Compound 569

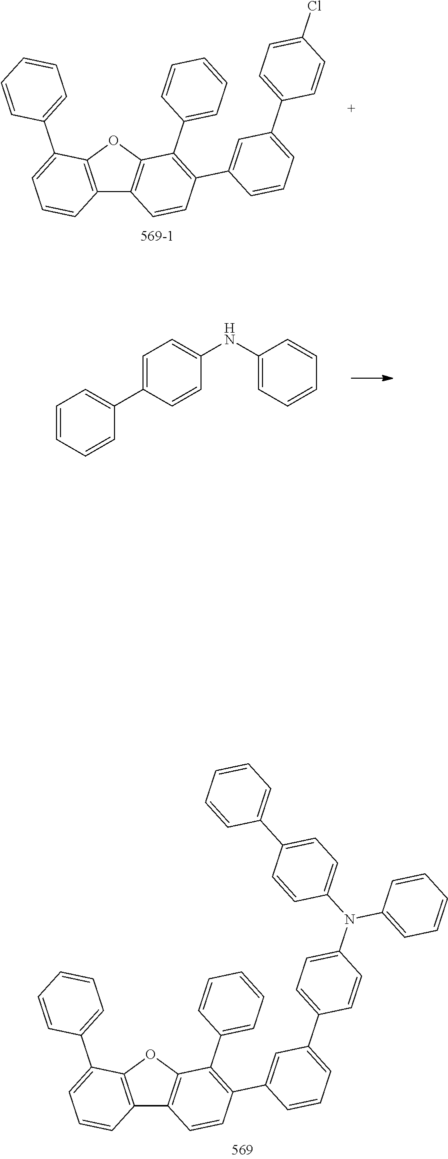 [SMILES: Clc1ccc(-c2cccc(-c3ccc4c(oc5c(-c6ccccc6)cccc54)c3-c3ccccc3)c2)cc1 ; c1ccc(Nc2ccc(-c3ccccc3)cc2)cc1 ; c1ccc(-c2ccc(N(c3ccccc3)c3ccc(-c4cccc(-c5ccc6c(oc7c(-c8ccccc8)cccc76)c5-c5ccccc5)c4)cc3)cc2)cc1]

150 mL of xylene was added to Intermediate 569-1 (10.0 g, 0.020 mol), N-phenyl[1,1′-biphenyl]-4-amine (7.3 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 9.6 g (yield 68.0%) of <Compound 569>.

LC/MS: m/z=715 [(M)⁺]

### Synthesis Example 37: Synthesis of Compound 579

### (1) Preparation Example 1: Synthesis of Compound 579

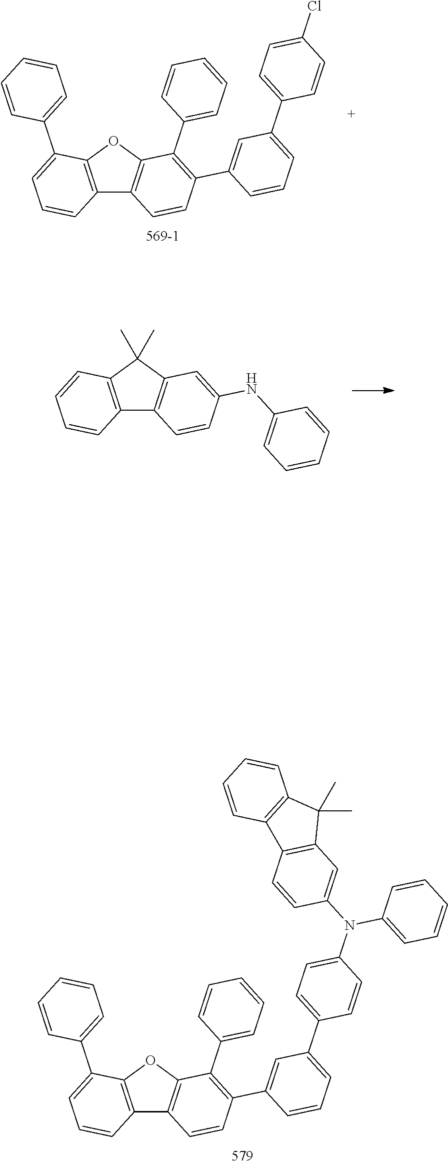 [SMILES: Clc1ccc(-c2cccc(-c3ccc4c(oc5c(-c6ccccc6)cccc54)c3-c3ccccc3)c2)cc1 ; CC1(C)c2ccccc2-c2ccc(Nc3ccccc3)cc21 ; CC1(C)c2ccccc2-c2ccc(N(c3ccccc3)c3ccc(-c4cccc(-c5ccc6c(oc7c(-c8ccccc8)cccc76)c5-c5ccccc5)c4)cc3)cc21]

150 mL of xylene was added to Intermediate 569-1 (10.0 g, 0.020 mol), 2-(phenylamino)-9,9-dimethylfluorene (8.4 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 10.2 g (yield 68.4%) of <Compound 579>.

LC/MS: m/z=755 [(M)⁺]

### Synthesis Example 38: Synthesis of Compound 598

### (1) Preparation Example 1: Synthesis of Compound 598

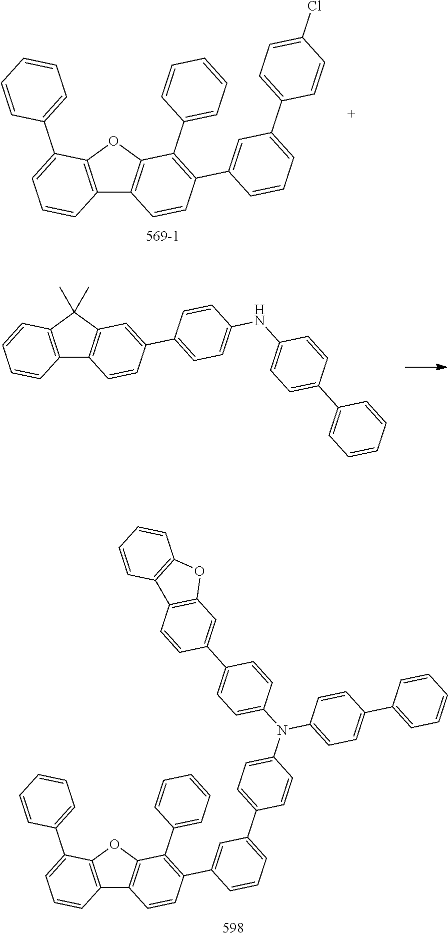 [SMILES: Clc1ccc(-c2cccc(-c3ccc4c(oc5c(-c6ccccc6)cccc54)c3-c3ccccc3)c2)cc1 ; CC1(C)c2ccccc2-c2ccc(-c3ccc(Nc4ccc(-c5ccccc5)cc4)cc3)cc21 ; c1ccc(-c2ccc(N(c3ccc(-c4cccc(-c5ccc6c(oc7c(-c8ccccc8)cccc76)c5-c5ccccc5)c4)cc3)c3ccc(-c4ccc5c(c4)oc4ccccc45)cc3)cc2)cc1]

150 mL of xylene was added to Intermediate 569-1 (10.0 g, 0.020 mol), N-[4-(3-dibenzofuranyl)phenyl][1,1′-biphenyl]-4-amine (12.2 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 10.0 g (yield 57.5%) of <Compound 598>.

LC/MS: m/z=881 [(M)⁺]

### Synthesis Example 39: Synthesis of Compound 616

### (1) Preparation Example 1: Synthesis of Intermediate 616-1

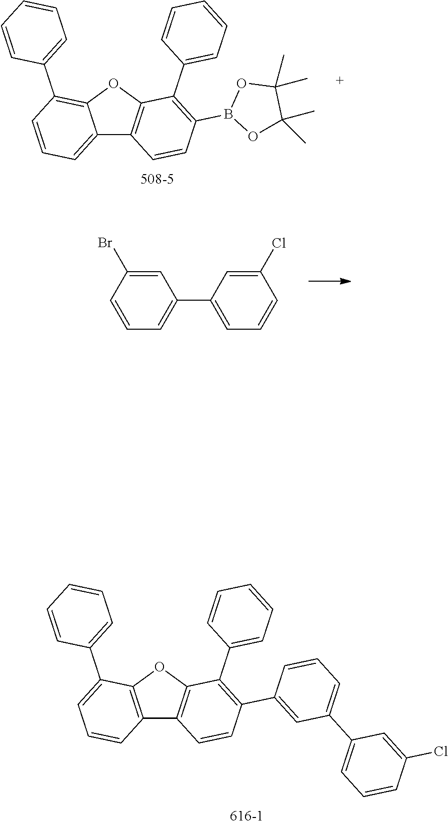 [SMILES: CC1(C)OB(c2ccc3c(oc4c(-c5ccccc5)cccc43)c2-c2ccccc2)OC1(C)C ; Clc1cccc(-c2cccc(Br)c2)c1 ; Clc1cccc(-c2cccc(-c3ccc4c(oc5c(-c6ccccc6)cccc54)c3-c3ccccc3)c2)c1]

200 mL of toluene, 50 mL of ethanol, and 50 mL of H₂O were added to Intermediate 508-5 (10.0 g, 0.022 mol), 3-bromo-3′-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.8 g (yield 59.9%) of <Intermediate 616-1>.

### (2) Preparation Example 2: Synthesis of Compound 616

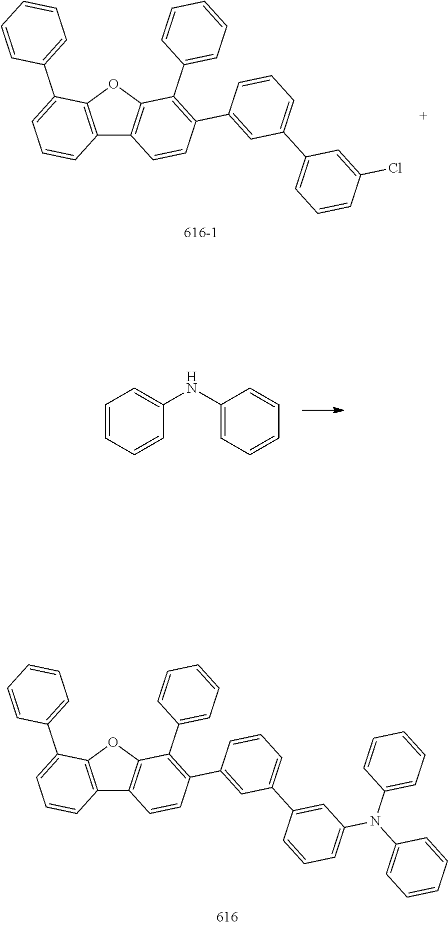 [SMILES: Clc1cccc(-c2cccc(-c3ccc4c(oc5c(-c6ccccc6)cccc54)c3-c3ccccc3)c2)c1 ; c1ccc(Nc2ccccc2)cc1 ; c1ccc(-c2cccc3c2oc2c(-c4ccccc4)c(-c4cccc(-c5cccc(N(c6ccccc6)c6ccccc6)c5)c4)ccc23)cc1]

150 mL of xylene was added to Intermediate 616-1 (10.0 g, 0.020 mol), diphenylamine (5.0 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 8.3 g (yield 65.8%) of <Compound 616>.

LC/MS: m/z=639 [(M)⁺]

### Synthesis Example 40: Synthesis of Compound 650

### (1) Preparation Example 1: Synthesis of Compound 650

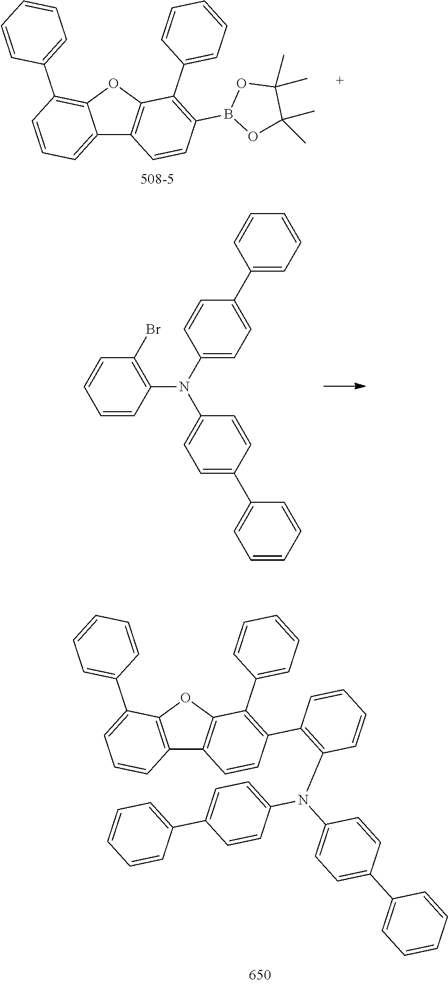 [SMILES: CC1(C)OB(c2ccc3c(oc4c(-c5ccccc5)cccc43)c2-c2ccccc2)OC1(C)C ; Brc1ccccc1N(c1ccc(-c2ccccc2)cc1)c1ccc(-c2ccccc2)cc1 ; c1ccc(-c2ccc(N(c3ccc(-c4ccccc4)cc3)c3ccccc3-c3ccc4c(oc5c(-c6ccccc6)cccc54)c3-c3ccccc3)cc2)cc1]

200 mL of toluene, 50 mL of ethanol, and 50 mL of H₂O were added to Intermediate 508-5 (10.0 g, 0.022 mol), N-[1,1′-biphenyl]-4-yl-N-(2-bromophenyl) [1,1′-biphenyl]-4-amine (12.8 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 9.0 g (yield 56.1%) of <Compound 650>.

LC/MS: m/z=715 [(M)⁺]

### Synthesis Example 41: Synthesis of Compound 682

### (1) Preparation Example 1: Synthesis of Intermediate 682-1

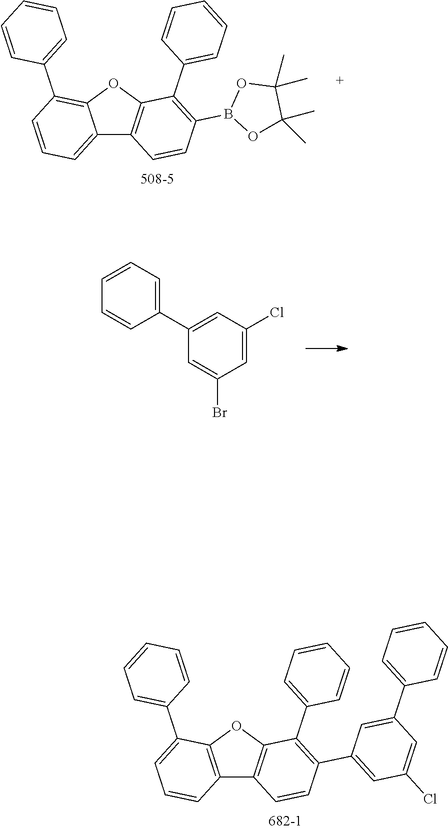 [SMILES: CC1(C)OB(c2ccc3c(oc4c(-c5ccccc5)cccc43)c2-c2ccccc2)OC1(C)C ; Clc1cc(Br)cc(-c2ccccc2)c1 ; Clc1cc(-c2ccccc2)cc(-c2ccc3c(oc4c(-c5ccccc5)cccc43)c2-c2ccccc2)c1]

200 mL of toluene, 50 mL of ethanol, and 50 mL of H₂O were added to Intermediate 508-5 (10.0 g, 0.022 mol), 3-bromo-5-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 5.7 g (yield 50.2%) of <Intermediate 682-1>.

### (2) Preparation Example 2: Synthesis of Compound 682

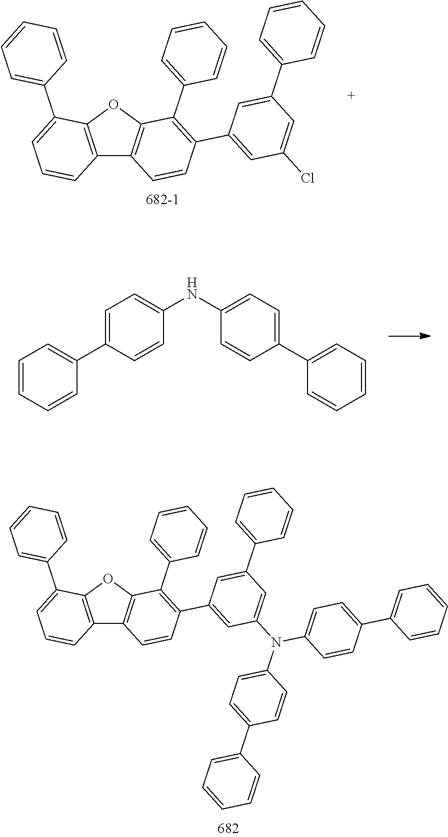 [SMILES: Clc1cc(-c2ccccc2)cc(-c2ccc3c(oc4c(-c5ccccc5)cccc43)c2-c2ccccc2)c1 ; c1ccc(-c2ccc(Nc3ccc(-c4ccccc4)cc3)cc2)cc1 ; c1ccc(-c2ccc(N(c3ccc(-c4ccccc4)cc3)c3cc(-c4ccccc4)cc(-c4ccc5c(oc6c(-c7ccccc7)cccc65)c4-c4ccccc4)c3)cc2)cc1]

150 mL of xylene was added to Intermediate 682-1 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 10.2 g (yield 65.3%) of <Compound 410>.

LC/MS: m/z=791 [(M)⁺]

### Synthesis Example 42: Synthesis of Compound 694

### (1) Preparation Example 1: Synthesis of Compound 694

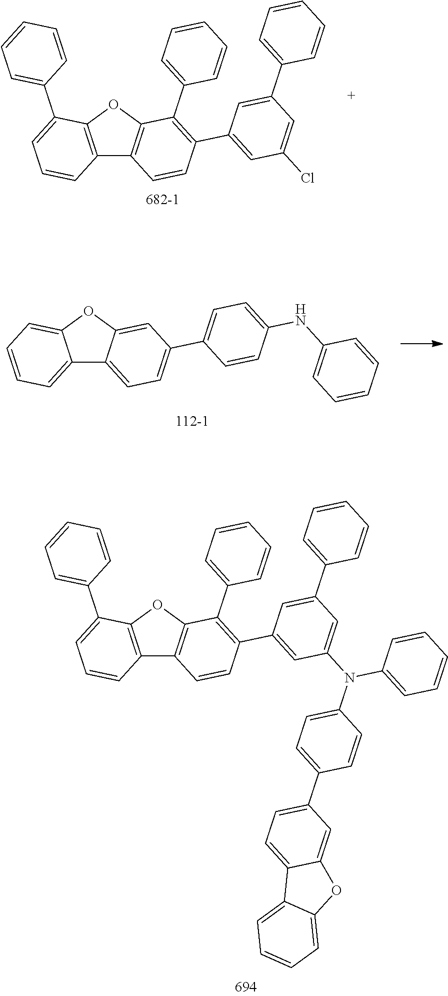 [SMILES: Clc1cc(-c2ccccc2)cc(-c2ccc3c(oc4c(-c5ccccc5)cccc43)c2-c2ccccc2)c1 ; c1ccc(Nc2ccc(-c3ccc4c(c3)oc3ccccc34)cc2)cc1 ; c1ccc(-c2cc(-c3ccc4c(oc5c(-c6ccccc6)cccc54)c3-c3ccccc3)cc(N(c3ccccc3)c3ccc(-c4ccc5c(c4)oc4ccccc45)cc3)c2)cc1]

150 mL of xylene was added to Intermediate 682-1 (10.0 g, 0.020 mol), Intermediate 112-1 (9.9 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 9.7 g (yield 61.0%) of <Compound 694>

LC/MS: m/z=805 [(M)⁺]

### Synthesis Example 43: Synthesis of Compound 708

### (1) Preparation Example 1: Synthesis of Intermediate 708-1

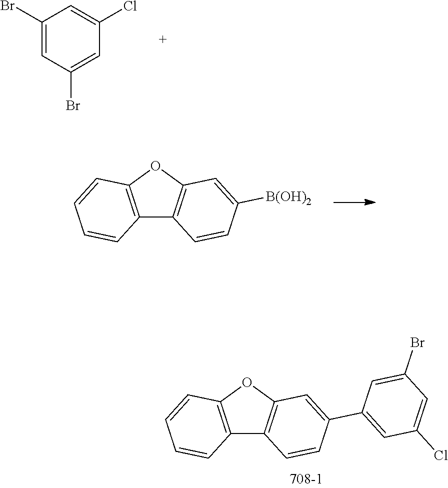 [SMILES: Clc1cc(Br)cc(Br)c1 ; OB(O)c1ccc2c(c1)oc1ccccc12 ; Clc1cc(Br)cc(-c2ccc3c(c2)oc2ccccc23)c1]

200 mL of toluene, 50 mL of ethanol, and 50 mL of H₂O were added to 1,3-dibromo-5-chlorobenzene (10.0 g, 0.037 mol), B-3-dibenzofuranylboronic acid (9.4 g, 0.044 mol), K₂CO₃ (15.3 g, 0.111 mol), and Pd(PPh₃)₄ (0.9 g, 0.7 mmol), and the resulting mixture was stirred at 80° C. for 6 hours and reacted. After completion of the reaction, the resulting product was extracted and concentrated, and then subjected to column chromatography to obtain 5.5 g (yield 41.6%) of <Intermediate 708-1>.

### (2) Preparation Example 2: Synthesis of Intermediate 708-2

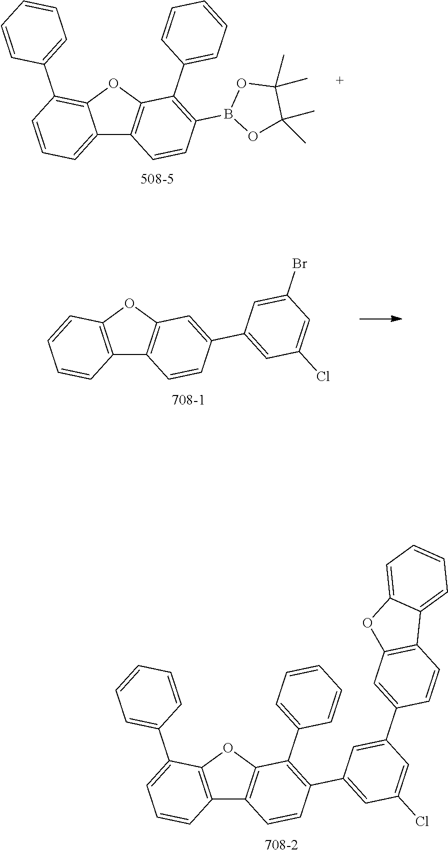 [SMILES: CC1(C)OB(c2ccc3c(oc4c(-c5ccccc5)cccc43)c2-c2ccccc2)OC1(C)C ; Clc1cc(Br)cc(-c2ccc3c(c2)oc2ccccc23)c1 ; Clc1cc(-c2ccc3c(c2)oc2ccccc23)cc(-c2ccc3c(oc4c(-c5ccccc5)cccc43)c2-c2ccccc2)c1]

200 mL of toluene, 50 mL of ethanol, and 50 mL of H₂O were added to Intermediate 508-5 (10.0 g, 0.022 mol), Intermediate 708-1 (9.6 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 7.4 g (yield 55.3%) of <Intermediate 708-2>.

### (3) Preparation Example 3: Synthesis of Compound 708

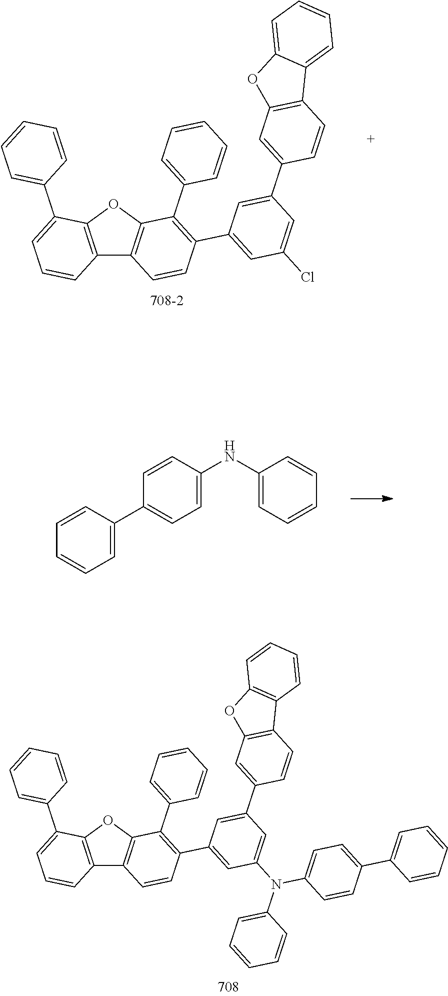 [SMILES: Clc1cc(-c2ccc3c(c2)oc2ccccc23)cc(-c2ccc3c(oc4c(-c5ccccc5)cccc43)c2-c2ccccc2)c1 ; c1ccc(Nc2ccc(-c3ccccc3)cc2)cc1 ; c1ccc(-c2ccc(N(c3ccccc3)c3cc(-c4ccc5c(c4)oc4ccccc45)cc(-c4ccc5c(oc6c(-c7ccccc7)cccc65)c4-c4ccccc4)c3)cc2)cc1]

150 mL of xylene was added to Intermediate 708-2 (10.0 g, 0.018 mol), N-phenyl[1,1′-biphenyl]-4-amine (6.2 g, 0.025 mol), NaOtBu (4.8 g, 0.050 mol), Pd(dba)₂ (0.4 g, 0.7 mmol), and t-Bu₃P (0.3 g, 1.3 mmol), and the resulting mixture was reacted under stirring at 120° C. for 4 hours. After completion of the reaction, the resulting product was extracted and concentrated, and then subjected to column chromatography and recrystallization to obtain 8.6 g (yield 63.7%) of <Compound 708>.

LC/MS: m/z=805 [(M)⁺]

### Synthesis Example 44: Synthesis of Compound 922

### (1) Preparation Example 1: Synthesis of Intermediate 922-1

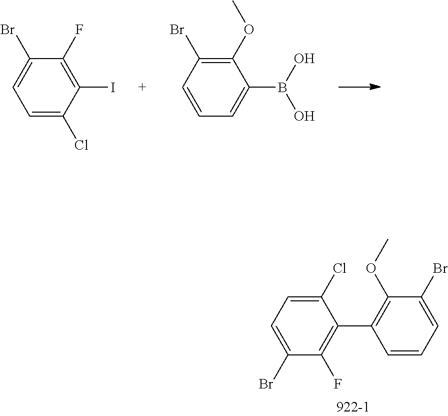

200 mL of toluene, 50 mL of ethanol, and 50 mL of H₂O were added to 3-bromo-6-chloro-3-fluoro-2-fluoroiodobenzene (10.0 g, 0.030 mol), B-(3-bromo-2-methoxyphenyl) boronic acid (8.3 g, 0.036 mol), K₂CO₃ (12.4 g, 0.090 mol), and Pd(PPh₃)₄ (0.7 g, 0.6 mmol), and the resulting mixture was reacted under stirring at 80° C. for 6 hours. After completion of the reaction, the resulting product was extracted and concentrated, and then subjected to column chromatography to obtain 5.2 g (yield 44.2%) of <Intermediate 922-1>.

### (2) Preparation Example 2: Synthesis of Intermediate 922-2

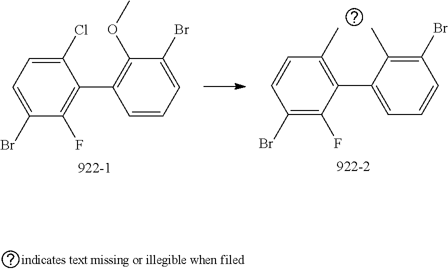 [SMILES: COc1c(Br)cccc1-c1c(Cl)ccc(Br)c1F ; Cc1ccc(Br)c(F)c1-c1cccc(Br)c1C]

Intermediate 922-1 (10.0 g, 0.025 mol) was dissolved in 60 mL of anhydrous DCM. Thereafter, BBr₃ (in DCM, 15.9 g, 0.063 mol) was added dropwise while maintaining the temperature at 0° C., and the resulting mixture was reacted under stirring at 25° C. for 16 hours. After completion of the reaction, the resulting product was extracted and concentrated, and then subjected to column chromatography to obtain 7.5 g (yield 77.8%) of <Intermediate 922-2>.

### (3) Preparation Example 3: Synthesis of Intermediate 922-3

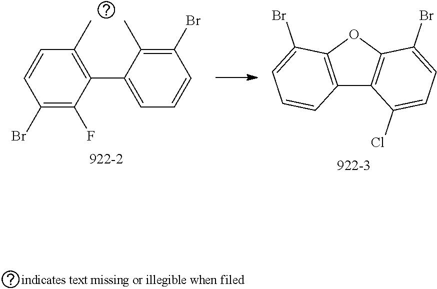 [SMILES: Cc1ccc(Br)c(F)c1-c1cccc(Br)c1C ; Clc1ccc(Br)c2oc3c(Br)cccc3c12]

500 mL of NMP was added to Intermediate 508-2 (10.0 g, 0.026 mol) and K₂CO₃ (22.6 g, 0.066 mol), and the resulting mixture was reacted under stirring at 180° C. for 24 hours. After completion of the reaction, the resulting product was extracted and concentrated, and then subjected to column chromatography to obtain 4.9 g (yield 51.7%) of <Intermediate 508-3>.

### (4) Preparation Example 4: Synthesis of Intermediate 922-4

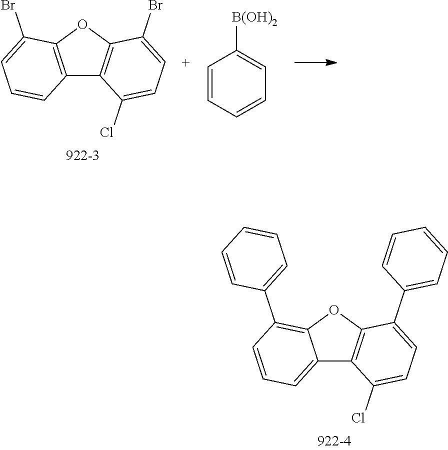

200 mL of toluene, 50 mL of ethanol, and 50 mL of H₂O were added to Intermediate 922-3 (10.0 g, 0.028 mol), phenylboronic acid (8.1 g, 0.067 mol), K₂CO₃ (23.0 g, 0.167 mol), and Pd(PPh₃)₄ (0.6 g, 0.6 mmol), and the resulting mixture was reacted under stirring at 80° C. for 6 hours. After completion of the reaction, the resulting product was extracted and concentrated, and then subjected to column chromatography to obtain 6.3 g (yield 64.0%) of <Intermediate 922-4>.

### (5) Preparation Example 5: Synthesis of Intermediate 922-5

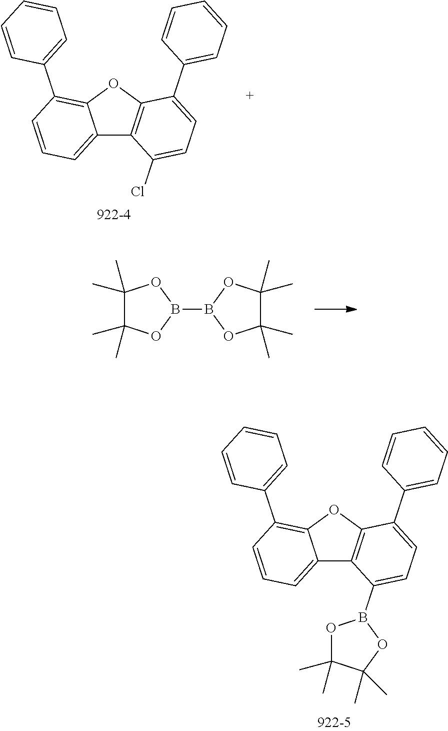

200 mL of dioxane was added to Intermediate 922-4 (10.0 g, 0.028 mol), bis(pinacolato)diboron (8.6 g, 0.034 mol), KOAc (8.3 g, 0.085 mol), and Pd(dppf)Cl₂ (1.0 g, 1.4 mmol), and the resulting mixture was reacted under stirring at 100° C. for 12 hours. After completion of the reaction, the resulting product was extracted and concentrated, and then subjected to column chromatography to obtain 7.8 g (yield 62.0%) of <Intermediate 922-5>.

### (6) Preparation Example 6: Synthesis of Intermediate 922-6

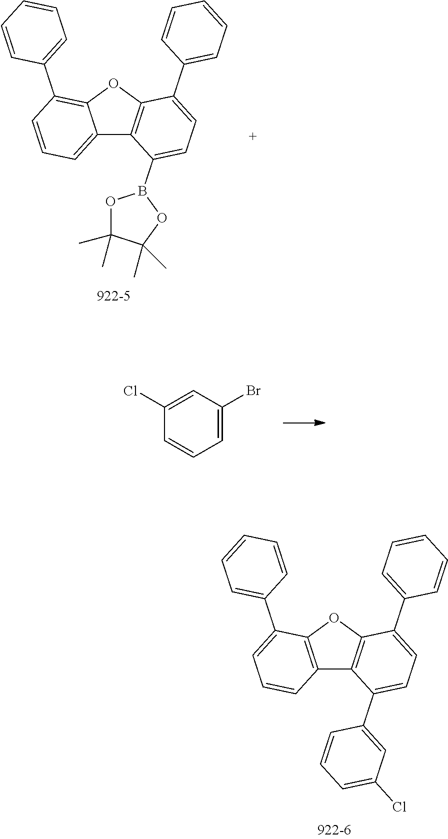 [SMILES: CC1(C)OB(c2ccc(-c3ccccc3)c3oc4c(-c5ccccc5)cccc4c23)OC1(C)C ; Clc1cccc(Br)c1 ; Clc1cccc(-c2ccc(-c3ccccc3)c3oc4c(-c5ccccc5)cccc4c23)c1]

200 mL of toluene, 50 mL of ethanol, and 50 mL of H₂O were added to Intermediate 922-5 (10.0 g, 0.022 mol), 1-bromo-3-chlorobenzene (5.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.0 g (yield 62.2%) of <Intermediate 922-6>.

### (7) Preparation Example 7: Synthesis of Compound 922

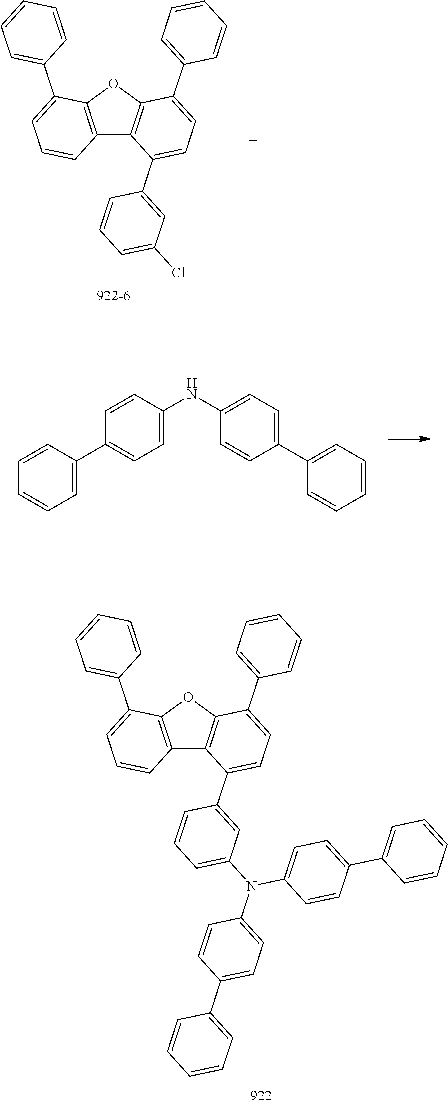 [SMILES: Clc1cccc(-c2ccc(-c3ccccc3)c3oc4c(-c5ccccc5)cccc4c23)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc(-c5ccccc5)c5oc6c(-c7ccccc7)cccc6c45)c3)cc2)cc1]

150 mL of xylene was added to Intermediate 922-6 (10.0 g, 0.023 mol), bis(4-biphenylyl)amine (11.2 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.8 g (yield 71.0%) of <Compound 922>.

LC/MS: m/z=715 [(M)⁺]

### Synthesis Example 45: Synthesis of Compound 937

### (1) Preparation Example 1: Synthesis of Compound 937

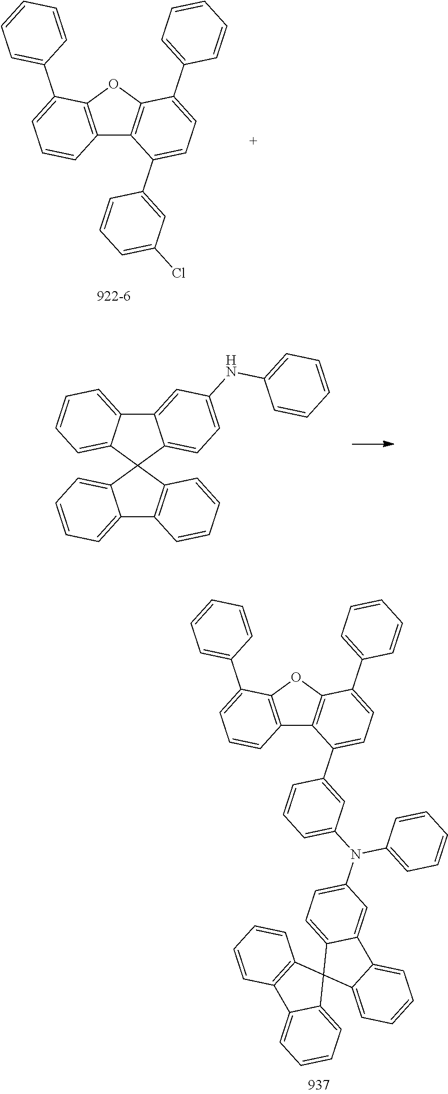 [SMILES: Clc1cccc(-c2ccc(-c3ccccc3)c3oc4c(-c5ccccc5)cccc4c23)c1 ; c1ccc(Nc2ccc3c(c2)-c2ccccc2C32c3ccccc3-c3ccccc32)cc1 ; c1ccc(-c2cccc3c2oc2c(-c4ccccc4)ccc(-c4cccc(N(c5ccccc5)c5ccc6c(c5)-c5ccccc5C65c6ccccc6-c6ccccc65)c4)c23)cc1]

150 mL of xylene was added to Intermediate 922-6 (10.0 g, 0.023 mol), N-phenyl-9,9′-spirobi[9H-fluoren]-3-amine (14.2 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.6 g (yield 62.3%) of <Compound 937>. LC/MS: m/z=801 [(M)⁺]

### Synthesis Example 46: Synthesis of Compound 947

### (1) Preparation Example 1: Synthesis of Compound 947

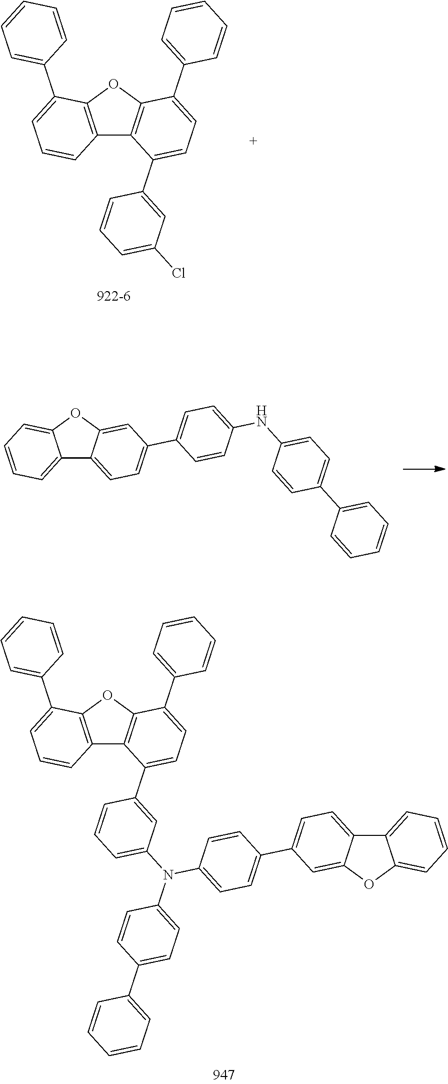 [SMILES: Clc1cccc(-c2ccc(-c3ccccc3)c3oc4c(-c5ccccc5)cccc4c23)c1 ; c1ccc(-c2ccc(Nc3ccc(-c4ccc5c(c4)oc4ccccc45)cc3)cc2)cc1 ; c1ccc(-c2ccc(N(c3ccc(-c4ccc5c(c4)oc4ccccc45)cc3)c3cccc(-c4ccc(-c5ccccc5)c5oc6c(-c7ccccc7)cccc6c45)c3)cc2)cc1]

150 mL of xylene was added to Intermediate 922-6 (10.0 g, 0.023 mol), N-[4-(3-dibenzofuranyl)phenyl][1,1′-biphenyl]-4-amine (14.3 g, 0.035 mol), NaOtBu (6.7 g, 0.070 mol), Pd(dba)₂ (0.5 g, 0.9 mmol), and t-Bu₃P (0.4 g, 1.9 mmol), and the resulting mixture was reacted under stirring at 120° C. for 4 hours. After completion of the reaction, the resulting product was extracted and concentrated, and then subjected to column chromatography and recrystallization to obtain 11.1 g (yield 59.4%) of <Compound 947>.

LC/MS: m/z=805 [(M)⁺]

### Synthesis Example 47: Synthesis of Compound 975

### (1) Preparation Example 1: Synthesis of Intermediate 975-1

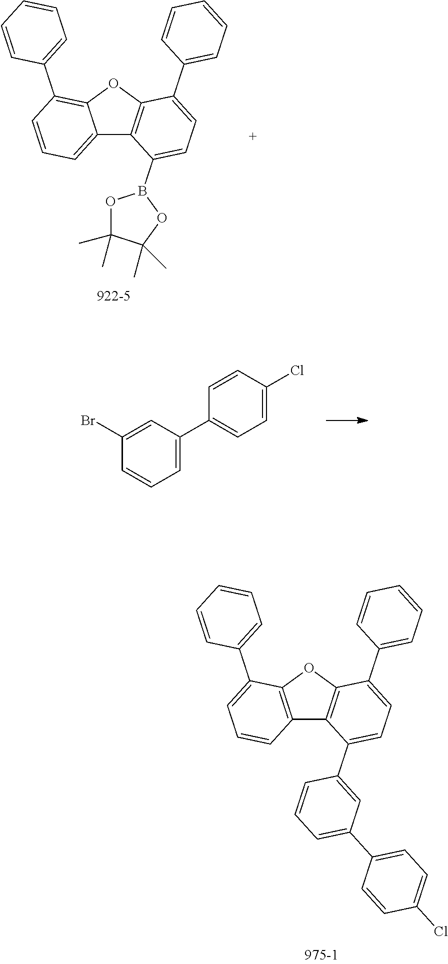 [SMILES: CC1(C)OB(c2ccc(-c3ccccc3)c3oc4c(-c5ccccc5)cccc4c23)OC1(C)C ; Clc1ccc(-c2cccc(Br)c2)cc1 ; Clc1ccc(-c2cccc(-c3ccc(-c4ccccc4)c4oc5c(-c6ccccc6)cccc5c34)c2)cc1]

200 mL of toluene, 50 mL of ethanol, and 50 mL of H₂O were added to Intermediate 922-5 (10.0 g, 0.022 mol), 3-bromo-4′-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.3 g (yield 55.5%) of <Intermediate 975-1>.

### (2) Preparation Example 2: Synthesis of Compound 975

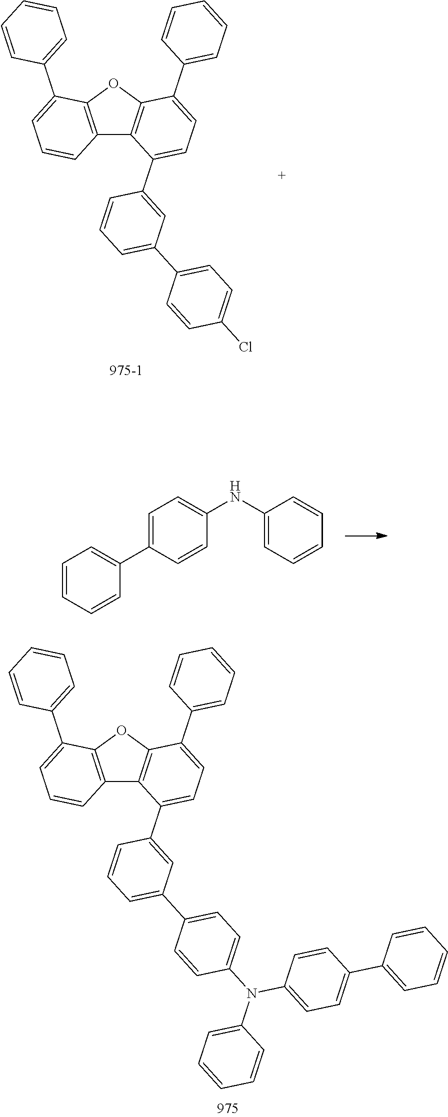 [SMILES: Clc1ccc(-c2cccc(-c3ccc(-c4ccccc4)c4oc5c(-c6ccccc6)cccc5c34)c2)cc1 ; c1ccc(Nc2ccc(-c3ccccc3)cc2)cc1 ; c1ccc(-c2ccc(N(c3ccccc3)c3ccc(-c4cccc(-c5ccc(-c6ccccc6)c6oc7c(-c8ccccc8)cccc7c56)c4)cc3)cc2)cc1]

150 mL of xylene was added to Intermediate 975-1 (10.0 g, 0.020 mol), N-phenyl[1,1′-biphenyl]-4-amine (7.3 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 8.5 g (yield 60.2%) of <Compound 975>.

LC/MS: m/z=715 [(M)⁺]

### Synthesis Example 48: Synthesis of Compound 1080

### (1) Preparation Example 1: Synthesis of Intermediate 1080-1

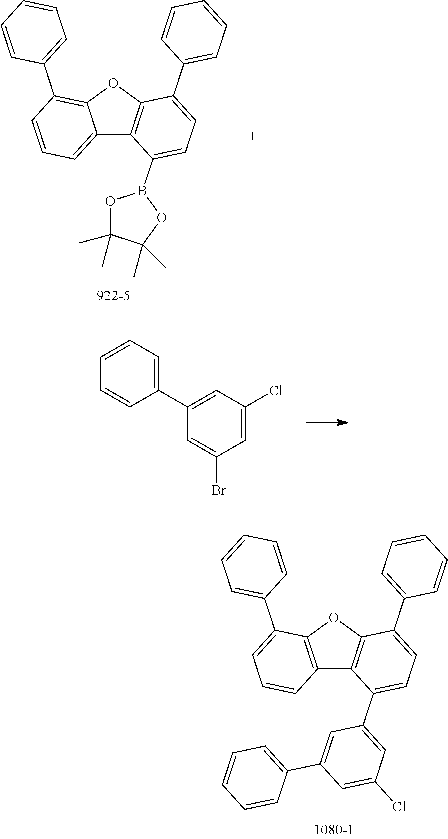 [SMILES: CC1(C)OB(c2ccc(-c3ccccc3)c3oc4c(-c5ccccc5)cccc4c23)OC1(C)C ; Clc1cc(Br)cc(-c2ccccc2)c1 ; Clc1cc(-c2ccccc2)cc(-c2ccc(-c3ccccc3)c3oc4c(-c5ccccc5)cccc4c23)c1]

200 mL of toluene, 50 mL of ethanol, and 50 mL of H₂O were added to Intermediate 922-5 (10.0 g, 0.022 mol), 3-bromo-5-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 6.4 g (yield 56.3%) of <Intermediate 1080-1>.

### (2) Preparation Example 2: Synthesis of Compound 1080

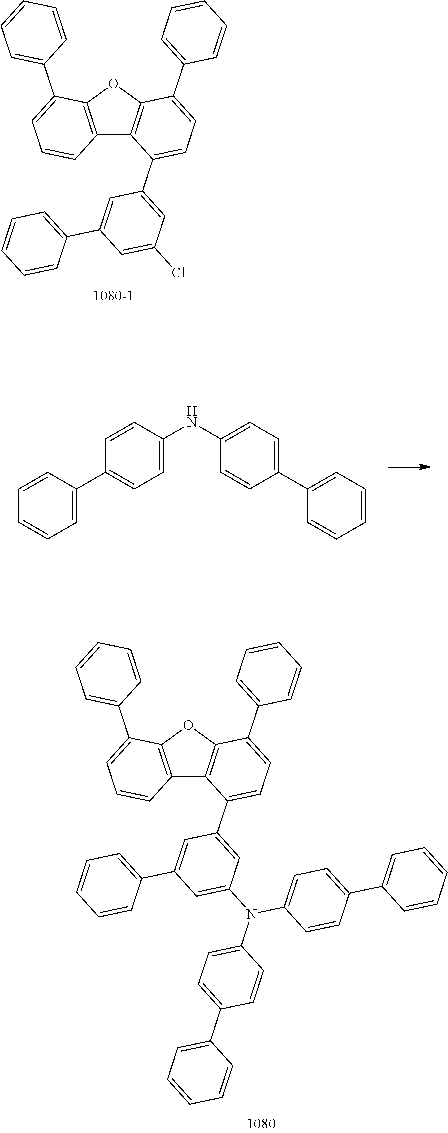 [SMILES: Clc1cc(-c2ccccc2)cc(-c2ccc(-c3ccccc3)c3oc4c(-c5ccccc5)cccc4c23)c1 ; c1ccc(-c2ccc(Nc3ccc(-c4ccccc4)cc3)cc2)cc1 ; c1ccc(-c2ccc(N(c3ccc(-c4ccccc4)cc3)c3cc(-c4ccccc4)cc(-c4ccc(-c5ccccc5)c5oc6c(-c7ccccc7)cccc6c45)c3)cc2)cc1]

150 mL of xylene was added to Intermediate 1080-1 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 9.7 g (yield 62.1%) of <Compound 1080>.

LC/MS: m/z=791 [(M)⁺]

### Synthesis Example 49: Synthesis of Compound 1102

### (1) Preparation Example 1: Synthesis of Intermediate 1102-1

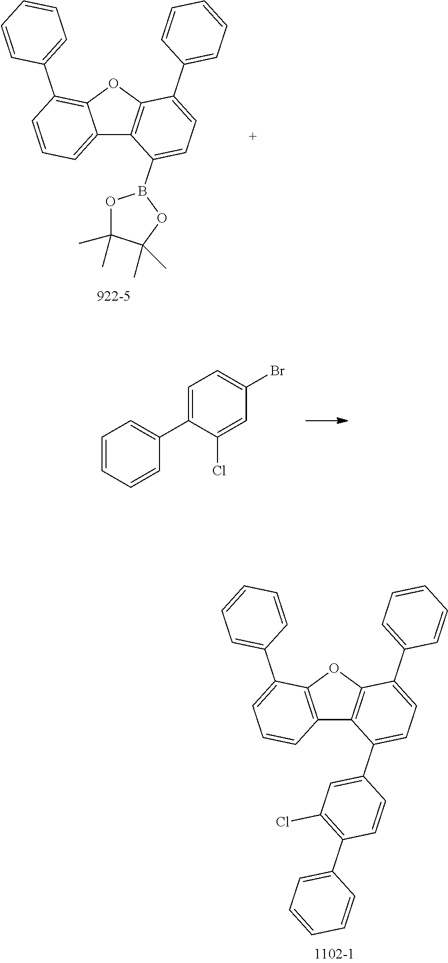 [SMILES: CC1(C)OB(c2ccc(-c3ccccc3)c3oc4c(-c5ccccc5)cccc4c23)OC1(C)C ; Clc1cc(Br)ccc1-c1ccccc1 ; Clc1cc(-c2ccc(-c3ccccc3)c3oc4c(-c5ccccc5)cccc4c23)ccc1-c1ccccc1]

200 mL of toluene, 50 mL of ethanol, and 50 mL of H₂O were added to Intermediate 922-5 (10.0 g, 0.022 mol), 4-bromo-2-chloro-1,1′-biphenyl (7.2 g, 0.027 mol), K₂CO₃ (9.3 g, 0.067 mol), and Pd(PPh₃)₄ (0.5 g, 0.4 mmol), and the resulting mixture was reacted under stirring at 80° C. for 6 hours. After completion of the reaction, the resulting product was extracted and concentrated, and then subjected to column chromatography and recrystallization to obtain 7.0 g (yield 61.6%) of <Intermediate 1102-1>.

### (2) Preparation Example 2: Synthesis of Compound 1102

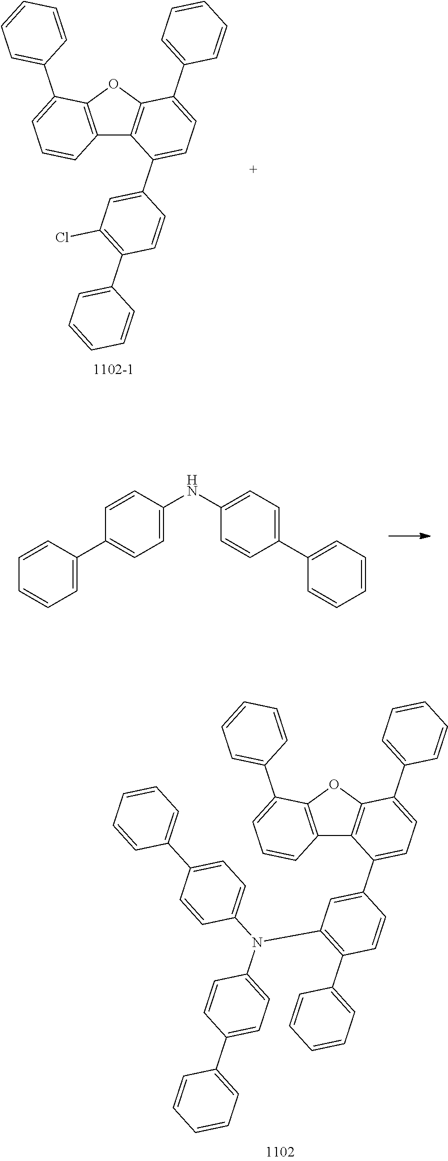 [SMILES: Clc1cc(-c2ccc(-c3ccccc3)c3oc4c(-c5ccccc5)cccc4c23)ccc1-c1ccccc1 ; c1ccc(-c2ccc(Nc3ccc(-c4ccccc4)cc3)cc2)cc1 ; c1ccc(-c2ccc(N(c3ccc(-c4ccccc4)cc3)c3cc(-c4ccc(-c5ccccc5)c5oc6c(-c7ccccc7)cccc6c45)ccc3-c3ccccc3)cc2)cc1]

150 mL of xylene was added to Intermediate 1102-1 (10.0 g, 0.020 mol), bis(4-biphenylyl)amine (9.5 g, 0.030 mol), NaOtBu (5.7 g, 0.059 mol), Pd(dba)₂ (0.5 g, 0.8 mmol), and t-Bu₃P (0.3 g, 1.6 mmol), and the resulting mixture was reacted under stirring at 120° C. for 4 hours. After completion of the reaction, the resulting product was extracted and concentrated, and then subjected to column chromatography and recrystallization to obtain 9.0 g (yield 57.6%) of <Compound 1102>.

LC/MS: m/z=791 [(M)⁺]

### Device Examples (EBL)

In exemplary embodiments according to the present invention, an ITO transparent electrode was patterned using an ITO glass substrate to which the ITO transparent electrode was attached on a glass substrate of 25 mm×25 mm×0.7 mm such that a light emitting area had a size of 2 mm×2 mm, and then washed. After the substrate was mounted in a vacuum chamber, a base pressure was set to 1×10⁻⁶ torr, and organic substances and a metal were deposited to have the following structure on the ITO.

### Device Examples 1 to 117

After an organic light emitting device having the following device structure was manufactured by employing a compound implemented by the present invention for an electron blocking layer, light emitting and driving characteristics of the compound implemented according to the present invention were measured.

ITO/hole injecting layer (HAT-CN, 5 nm)/hole transport layer (HT1, 100 nm)/electron blocking layer (10 nm)/light emitting layer (20 nm)/electron transport layer (ET1:Liq, 30 nm)/LiF (1 nm)/Al (100 nm)

After [HAT-CN] was film-formed to a thickness of 5 nm on top of an ITO transparent electrode to form a hole injecting layer, [HT1] was film-formed to a thickness of 100 nm to form a hole transport layer, the compound according to the present invention shown in the following Table 1 was film-formed to a thickness of 10 nm to form an electron blocking layer, and a light-emitting layer was formed by co-depositing [BH1] as a host compound and [BD1] as a dopant compound to a thickness of 20 nm. Thereafter, an electron transport layer (50% doping of the following [ET1] compound Liq) was deposited to a thickness of 30 nm, and then LiF was film-formed to a thickness of 1 nm to form an electron injecting layer, and Al was film-formed to a thickness of 100 nm, thereby manufacturing an organic light-emitting device.

### Device Comparative Example 1

An organic light emitting device for Device Comparative Example 2 was manufactured in the same manner as in the device structure in Examples 1 to 117, except that in the electron blocking layer, the following [EB1] was used instead of the compound according to the present invention.

### Device Comparative Example 2

An organic light emitting device for Device Comparative Example 2 was manufactured in the same manner as in the device structure in Examples 1 to 117, except that in the electron blocking layer, the following [EB2] was used instead of the compound according to the present invention.

### Experimental Example 1: Light-Emitting Characteristics of Device Examples 1 to 117

For the organic light-emitting devices manufactured by the Examples and the Comparative Examples, driving voltage, current efficiency and color coordinate were measured using a source meter (Model237, Keithley) and a luminance meter (PR-650, Photo Research), and the result values based on 1,000 nits are shown in the following [Table 1].

TABLE 1

| | Electron | | | | |
|---|---|---|---|---|---|
| Example | blocking layer | V | cd/A | CIEx | CIEy |
| 1 | Formula 5 | 4.20 | 7.84 | 0.1350 | 0.1334 |
| 2 | Formula 6 | 4.08 | 7.61 | 0.1340 | 0.1329 |
| 3 | Formula 11 | 4.19 | 7.71 | 0.1351 | 0.1322 |
| 4 | Formula 19 | 4.02 | 7.46 | 0.1338 | 0.1395 |
| 5 | Formula 21 | 4.18 | 7.81 | 0.1345 | 0.1360 |
| 6 | Formula 26 | 4.20 | 7.66 | 0.1331 | 0.1401 |
| 7 | Formula 35 | 4.20 | 7.84 | 0.1350 | 0.1334 |
| 8 | Formula 41 | 3.98 | 7.79 | 0.1348 | 0.1348 |
| 9 | Formula 52 | 4.20 | 7.82 | 0.1341 | 0.1311 |
| 10 | Formula 54 | 4.17 | 7.65 | 0.1345 | 0.1397 |
| 11 | Formula 68 | 4.27 | 7.57 | 0.1362 | 0.1342 |
| 12 | Formula 73 | 4.27 | 7.57 | 0.1362 | 0.1342 |
| 13 | Formula 76 | 4.24 | 7.52 | 0.1357 | 0.1325 |
| 14 | Formula 81 | 4.06 | 7.77 | 0.1360 | 0.1326 |
| 15 | Formula 83 | 4.15 | 7.48 | 0.1344 | 0.1391 |
| 16 | Formula 89 | 4.25 | 7.43 | 0.1338 | 0.1379 |
| 17 | Formula 91 | 4.01 | 7.84 | 0.1333 | 0.1346 |
| 18 | Formula 98 | 4.21 | 7.67 | 0.1335 | 0.1366 |
| 19 | Formula 105 | 4.02 | 7.94 | 0.1348 | 0.1336 |
| 20 | Formula 110 | 4.15 | 7.81 | 0.1368 | 0.1327 |
| 21 | Formula 111 | 4.21 | 7.60 | 0.1334 | 0.1386 |
| 22 | Formula 112 | 4.11 | 7.80 | 0.1348 | 0.1363 |
| 23 | Formula 117 | 4.20 | 7.96 | 0.1360 | 0.1334 |
| 24 | Formula 119 | 4.17 | 7.64 | 0.1344 | 0.1368 |
| 25 | Formula 121 | 4.15 | 7.81 | 0.1368 | 0.1327 |
| 26 | Formula 124 | 4.23 | 7.94 | 0.1344 | 0.1353 |
| 27 | Formula 129 | 4.25 | 7.43 | 0.1338 | 0.1379 |
| 28 | Formula 133 | 4.11 | 7.80 | 0.1348 | 0.1363 |
| 29 | Formula 145 | 4.29 | 7.53 | 0.1354 | 0.1317 |
| 30 | Formula 146 | 4.24 | 7.66 | 0.1351 | 0.1352 |
| 31 | Formula 147 | 4.06 | 7.77 | 0.1360 | 0.1326 |
| 32 | Formula 162 | 4.18 | 7.47 | 0.1354 | 0.1380 |
| 33 | Formula 165 | 4.30 | 7.51 | 0.1347 | 0.1337 |
| 34 | Formula 166 | 4.21 | 7.60 | 0.1334 | 0.1386 |
| 35 | Formula 170 | 4.25 | 7.52 | 0.1348 | 0.1361 |
| 36 | Formula 179 | 4.29 | 7.91 | 0.1342 | 0.1363 |
| 37 | Formula 180 | 4.12 | 7.61 | 0.1354 | 0.1358 |
| 38 | Formula 184 | 4.24 | 7.66 | 0.1351 | 0.1352 |
| 39 | Formula 190 | 4.25 | 7.52 | 0.1348 | 0.1361 |
| 40 | Formula 193 | 4.23 | 7.90 | 0.1368 | 0.1342 |
| 41 | Formula 195 | 4.14 | 7.57 | 0.1357 | 0.1338 |
| 42 | Formula 218 | 4.20 | 7.67 | 0.1336 | 0.1370 |
| 43 | Formula 229 | 4.14 | 7.55 | 0.1330 | 0.1374 |
| 44 | Formula 244 | 4.28 | 7.61 | 0.1340 | 0.1319 |
| 45 | Formula 259 | 4.05 | 7.52 | 0.1348 | 0.1361 |
| 46 | Formula 262 | 4.20 | 7.45 | 0.1355 | 0.1347 |
| 47 | Formula 264 | 4.05 | 7.52 | 0.1348 | 0.1361 |
| 48 | Formula 270 | 4.19 | 7.71 | 0.1351 | 0.1322 |
| 49 | Formula 277 | 4.15 | 7.59 | 0.1346 | 0.1337 |
| 50 | Formula 290 | 4.23 | 7.63 | 0.1337 | 0.1334 |
| 51 | Formula 294 | 4.11 | 7.45 | 0.1318 | 0.1355 |
| 52 | Formula 316 | 4.22 | 7.83 | 0.1344 | 0.1360 |
| 53 | Formula 338 | 4.24 | 7.74 | 0.1356 | 0.1360 |
| 54 | Formula 348 | 4.25 | 7.61 | 0.1345 | 0.1327 |
| 55 | Formula 356 | 4.15 | 7.82 | 0.1363 | 0.1341 |
| 56 | Formula 389 | 4.17 | 7.52 | 0.1349 | 0.1336 |
| 57 | Formula 410 | 4.13 | 7.80 | 0.1360 | 0.1327 |
| 58 | Formula 421 | 4.28 | 7.65 | 0.1352 | 0.1337 |
| 59 | Formula 431 | 4.32 | 7.71 | 0.1363 | 0.1346 |
| 60 | Formula 447 | 4.20 | 7.42 | 0.1388 | 0.1328 |
| 61 | Formula 468 | 4.13 | 7.80 | 0.1387 | 0.1347 |
| 62 | Formula 503 | 4.10 | 7.88 | 0.1363 | 0.1353 |
| 63 | Formula 508 | 4.09 | 7.79 | 0.1364 | 0.1338 |
| 64 | Formula 513 | 4.21 | 7.62 | 0.1341 | 0.1311 |
| 65 | Formula 518 | 4.16 | 7.60 | 0.1351 | 0.1352 |
| 66 | Formula 525 | 4.25 | 7.94 | 0.1388 | 0.1325 |
| 67 | Formula 537 | 4.18 | 7.63 | 0.1368 | 0.1332 |
| 68 | Formula 538 | 4.23 | 7.55 | 0.1371 | 0.1338 |
| 69 | Formula 539 | 4.14 | 7.64 | 0.1346 | 0.1340 |
| 70 | Formula 547 | 4.25 | 7.52 | 0.1340 | 0.1354 |
| 71 | Formula 555 | 4.09 | 7.79 | 0.1364 | 0.1338 |
| 72 | Formula 569 | 4.13 | 7.75 | 0.1363 | 0.1341 |
| 73 | Formula 570 | 4.10 | 7.92 | 0.1354 | 0.1352 |
| 74 | Formula 579 | 4.15 | 7.64 | 0.1367 | 0.1344 |
| 75 | Formula 586 | 4.12 | 7.72 | 0.1351 | 0.1353 |
| 76 | Formula 598 | 4.03 | 7.53 | 0.1361 | 0.1335 |
| 77 | Formula 616 | 4.10 | 7.76 | 0.1363 | 0.1341 |
| 78 | Formula 636 | 4.25 | 7.62 | 0.1358 | 0.1340 |
| 79 | Formula 650 | 4.14 | 7.68 | 0.1360 | 0.1349 |
| 80 | Formula 658 | 4.24 | 8.03 | 0.1361 | 0.1325 |
| 81 | Formula 682 | 4.06 | 7.77 | 0.1350 | 0.1326 |
| 82 | Formula 689 | 4.35 | 7.68 | 0.1344 | 0.1391 |
| 83 | Formula 694 | 4.14 | 7.57 | 0.1354 | 0.1380 |
| 84 | Formula 708 | 4.25 | 7.49 | 0.1328 | 0.1379 |
| 85 | Formula 757 | 4.18 | 7.45 | 0.1349 | 0.1336 |
| 86 | Formula 763 | 4.26 | 7.54 | 0.1345 | 0.1391 |
| 87 | Formula 811 | 4.20 | 7.67 | 0.1335 | 0.1386 |
| 88 | Formula 841 | 4.31 | 7.54 | 0.1330 | 0.1374 |
| 89 | Formula 871 | 4.17 | 7.62 | 0.1344 | 0.1368 |
| 90 | Formula 922 | 4.15 | 7.81 | 0.1361 | 0.1327 |
| 91 | Formula 930 | 4.21 | 7.60 | 0.1336 | 0.1386 |
| 92 | Formula 937 | 4.21 | 7.60 | 0.1330 | 0.1366 |
| 93 | Formula 945 | 4.29 | 7.53 | 0.1394 | 0.1327 |
| 94 | Formula 947 | 4.24 | 7.61 | 0.1351 | 0.1352 |
| 95 | Formula 974 | 4.36 | 7.52 | 0.1345 | 0.1371 |
| 96 | Formula 975 | 4.21 | 7.67 | 0.1334 | 0.1365 |
| 97 | Formula 983 | 4.20 | 7.63 | 0.1362 | 0.1376 |
| 98 | Formula 995 | 4.17 | 7.74 | 0.1344 | 0.1368 |
| 99 | Formula 1001 | 4.15 | 7.81 | 0.1358 | 0.1327 |
| 100 | Formula 1009 | 4.21 | 7.67 | 0.1334 | 0.1365 |
| 101 | Formula 1053 | 4.35 | 7.38 | 0.1344 | 0.1391 |
| 102 | Formula 1080 | 4.18 | 7.47 | 0.1354 | 0.1380 |
| 103 | Formula 1102 | 4.25 | 7.43 | 0.1329 | 0.1379 |
| 104 | Formula 1107 | 4.28 | 7.45 | 0.1338 | 0.1336 |
| 105 | Formula 1149 | 4.36 | 7.58 | 0.1345 | 0.1391 |
| 106 | Formula 1150 | 4.21 | 7.52 | 0.1340 | 0.1386 |
| 107 | Formula 1169 | 4.21 | 7.67 | 0.1335 | 0.1386 |
| 108 | Formula 1203 | 4.17 | 7.44 | 0.1344 | 0.1368 |
| 109 | Formula 1246 | 4.22 | 7.38 | 0.1341 | 0.1363 |
| 110 | Formula 1269 | 4.27 | 7.44 | 0.1339 | 0.1356 |
| 111 | Formula 1293 | 4.33 | 7.36 | 0.1340 | 0.1377 |
| 112 | Formula 1333 | 4.38 | 7.42 | 0.1329 | 0.1360 |
| 113 | Formula 1355 | 4.25 | 7.45 | 0.1354 | 0.1372 |
| 114 | Formula 1376 | 4.36 | 7.37 | 0.1336 | 0.1381 |
| 115 | Formula 1387 | 4.23 | 7.31 | 0.1344 | 0.1342 |
| 116 | Formula 1418 | 4.28 | 7.33 | 0.1349 | 0.1367 |
| 117 | Formula 1427 | 4.41 | 7.42 | 0.1337 | 0.1379 |
| Comparative | EB1 | 4.67 | 6.65 | 0.1353 | 0.1517 |
| Example 1 | | | | | |
| Comparative | EB2 | 4.59 | 6.91 | 0.1346 | 0.1458 |
| Example 2 | | | | | |

Referring to the results shown in [Table 1] above, it can be confirmed that when the compound according to the present invention is employed to an electron blocking layer in the organic light-emitting device, low-voltage driving characteristics and luminescent characteristics such as luminous efficiency and quantum efficiency are significantly excellent compared to the devices (Comparative Examples 1 to 2) in which compounds used as conventional materials for an electron blocking layer, which are contrasted with the characteristic structures of the compounds according to the present invention, were employed.

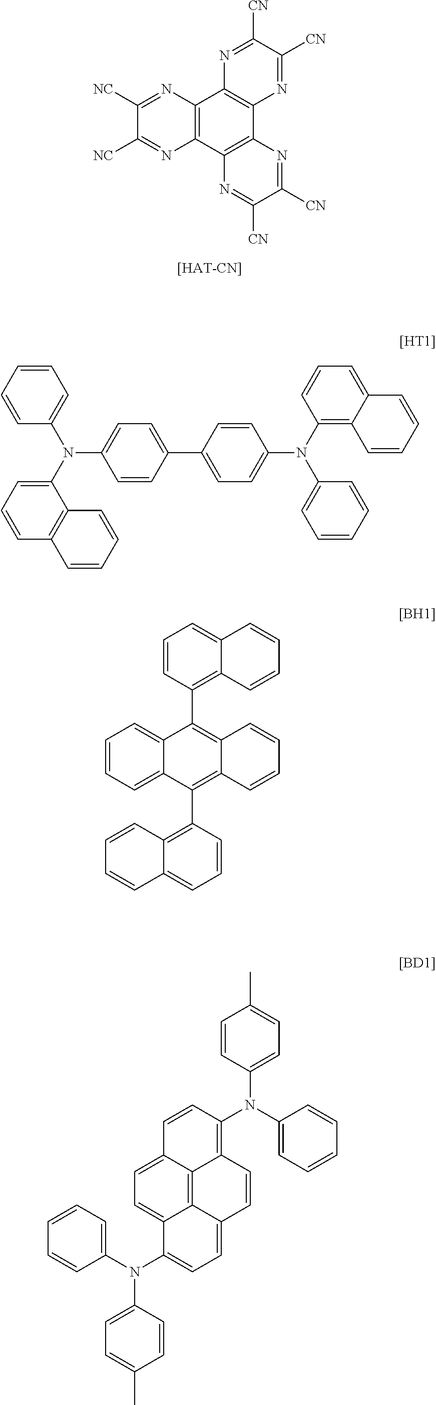

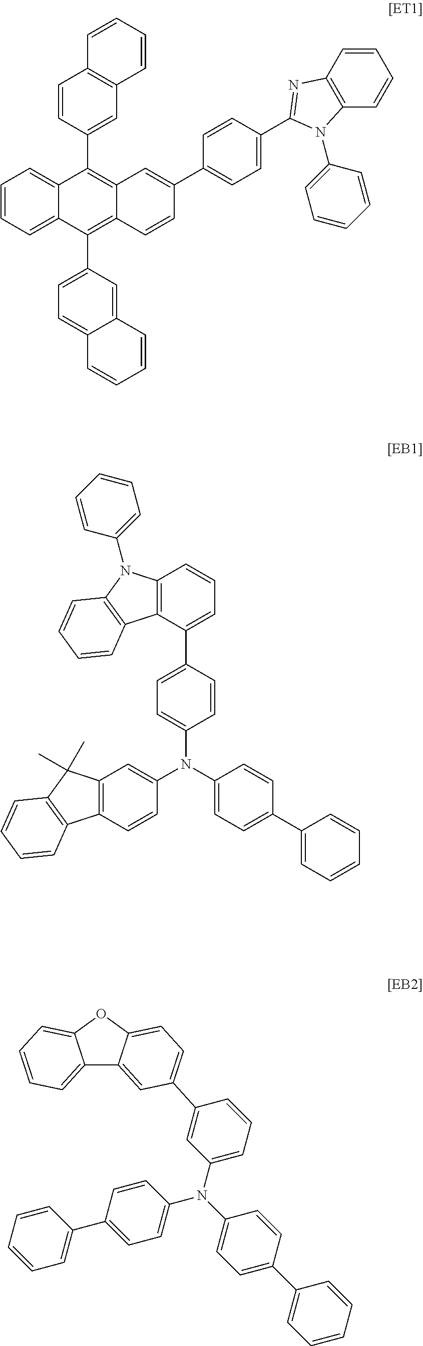 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2ccccc2-c2ccc(N(c3ccc(-c4ccccc4)cc3)c3ccc(-c4cccc5c4c4ccccc4n5-c4ccccc4)cc3)cc21 ; c1ccc(-c2ccc(N(c3ccc(-c4ccccc4)cc3)c3cccc(-c4ccc5oc6ccccc6c5c4)c3)cc2)cc1]

The present invention relates to an organic compound employed as an organic layer material such as an electron blocking layer in an organic light-emitting device. When the compound according to the present invention is employed for an electron-blocking layer in a device, it is possible to implement an organic light-emitting device having significantly improved low voltage driving characteristics and device characteristics such as long lifespan and luminous efficiency, and thus the present invention can be industrially useful for various lighting devices and display devices.

What is claimed is:

1. An organic compound represented by the following [Formula I]: 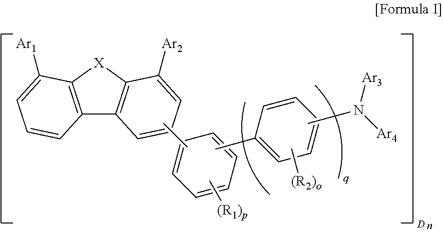
wherein, in [Formula I] above,
X is O or S,
Ar₁ and Ar₂ are each independently any one selected from hydrogen, a substituted or unsubstituted aryl group having 6 to 20 carbon atoms, and a substituted or unsubstituted heteroaryl group having 3 to 30 carbon atoms (provided that a case where both Ar₁ and Ar₂ are hydrogen is excluded),
Ar₃ and Ar₄ are each independently any one selected from a substituted or unsubstituted aryl group having 6 to 20 carbon atoms and a substituted or unsubstituted heteroaryl group having 3 to 30 carbon atoms,
R₁ and R₂ are the same as or different from each other, and are each independently hydrogen or deuterium, or any one selected from a substituted or unsubstituted aryl group having 6 to 20 carbon atoms and a substituted or unsubstituted heteroaryl group having 3 to 30 carbon atoms,
p and o are each an integer from 0 to 4, and when p and o are 2 or higher, a plurality of R₁ and R₂ are each the same as or different from each other,
q is an integer from 0 to 2, and when q is 2, the structures in a plurality of ( ) are the same as or different from each other, and
D is deuterium, n means the number of hydrogen atoms in [Formula I] above, which are replaced with deuterium atoms (D), and n is an integer from 0 to 60.

2. The organic compound of claim 1, wherein in the definitions of R₁, R₂, and Ar₁ to Ar₄, substituted or unsubstituted means that each of R₁, R₂, and Ar₁ to Ar₄ is substituted with one or two or more substituents selected from deuterium, a cyano group, a halogen group, a hydroxyl group, a nitro group, an alkyl group, an alkoxy group, a halogenated alkoxy group, a cycloalkyl group, a heterocycloalkyl group, an aryl group, a fluorenyl group, a heteroaryl group, and a silyl group, is substituted with a substituent to which two or more substituents among the above substituents are linked, or has no substituent.

3. The organic compound of claim 1, wherein the organic compound represented by [Formula I] comprises at least one deuterium atom in in the [Formula I] structure.

4. The organic compound of claim 1, wherein [Formula I] above is a compound in which hydrogen present in [Formula I] is partially substituted with deuterium (D), and the deuterium (D) substitution rate is 10 to 90%.

5. The organic compound of claim 3, wherein the deuterium (D) substitution rate is 20 to 80%.

6. The organic compound of claim 4, wherein the deuterium (D) substitution rate is 30 to 70%.

7. The organic compound of claim 1, wherein [Formula I] above is any one selected from among [Formula 1] to [Formula 1450] below:

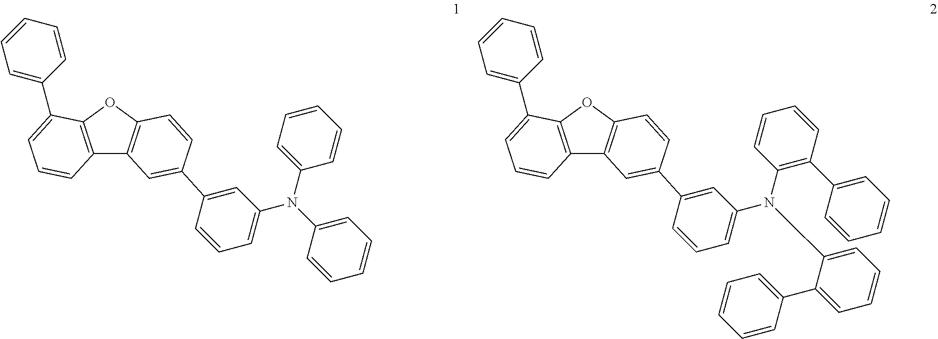 [SMILES: c1ccc(-c2cccc3c2oc2ccc(-c4cccc(N(c5ccccc5)c5ccccc5)c4)cc23)cc1 ; c1ccc(-c2ccccc2N(c2cccc(-c3ccc4oc5c(-c6ccccc6)cccc5c4c3)c2)c2ccccc2-c2ccccc2)cc1]

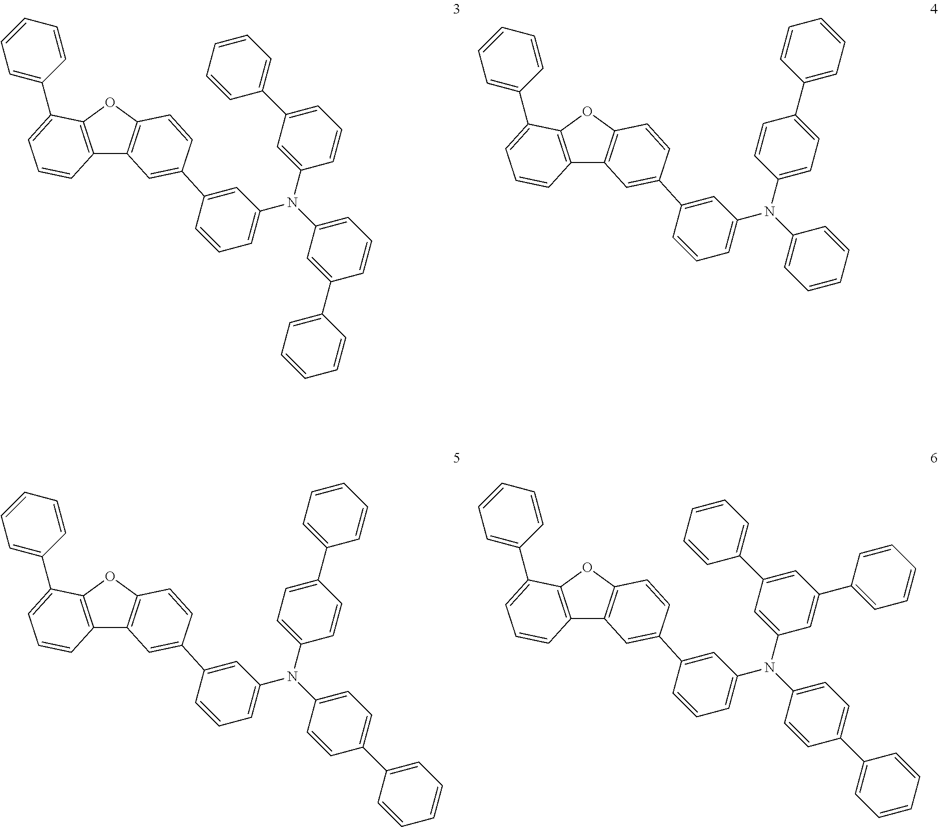 [SMILES: c1ccc(-c2cccc(N(c3cccc(-c4ccccc4)c3)c3cccc(-c4ccc5oc6c(-c7ccccc7)cccc6c5c4)c3)c2)cc1 ; c1ccc(-c2ccc(N(c3ccccc3)c3cccc(-c4ccc5oc6c(-c7ccccc7)cccc6c5c4)c3)cc2)cc1 ; c1ccc(-c2ccc(N(c3ccc(-c4ccccc4)cc3)c3cccc(-c4ccc5oc6c(-c7ccccc7)cccc6c5c4)c3)cc2)cc1 ; c1ccc(-c2ccc(N(c3cccc(-c4ccc5oc6c(-c7ccccc7)cccc6c5c4)c3)c3cc(-c4ccccc4)cc(-c4ccccc4)c3)cc2)cc1]

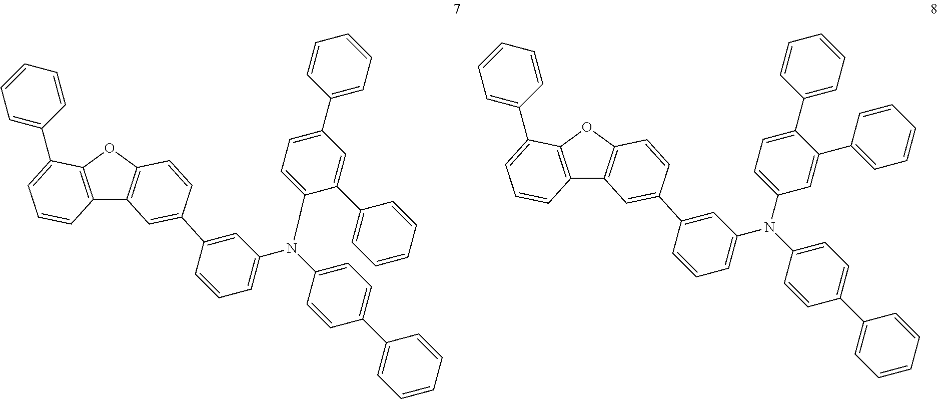 [SMILES: c1ccc(-c2ccc(N(c3cccc(-c4ccc5oc6c(-c7ccccc7)cccc6c5c4)c3)c3ccc(-c4ccccc4)cc3-c3ccccc3)cc2)cc1 ; c1ccc(-c2ccc(N(c3cccc(-c4ccc5oc6c(-c7ccccc7)cccc6c5c4)c3)c3ccc(-c4ccccc4)c(-c4ccccc4)c3)cc2)cc1]

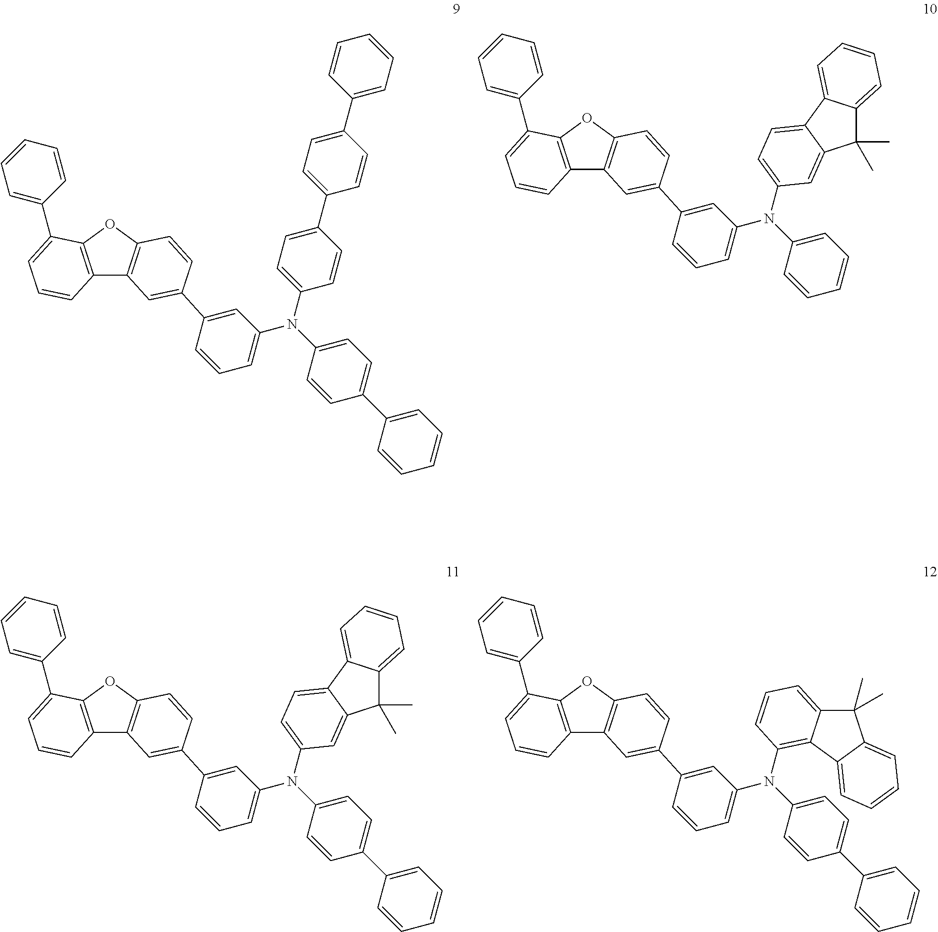 [SMILES: c1ccc(-c2ccc(-c3ccc(N(c4ccc(-c5ccccc5)cc4)c4cccc(-c5ccc6oc7c(-c8ccccc8)cccc7c6c5)c4)cc3)cc2)cc1 ; CC1(C)c2ccccc2-c2ccc(N(c3ccccc3)c3cccc(-c4ccc5oc6c(-c7ccccc7)cccc6c5c4)c3)cc21 ; CC1(C)c2ccccc2-c2ccc(N(c3ccc(-c4ccccc4)cc3)c3cccc(-c4ccc5oc6c(-c7ccccc7)cccc6c5c4)c3)cc21 ; CC1(C)c2ccccc2-c2c(N(c3ccc(-c4ccccc4)cc3)c3cccc(-c4ccc5oc6c(-c7ccccc7)cccc6c5c4)c3)cccc21]

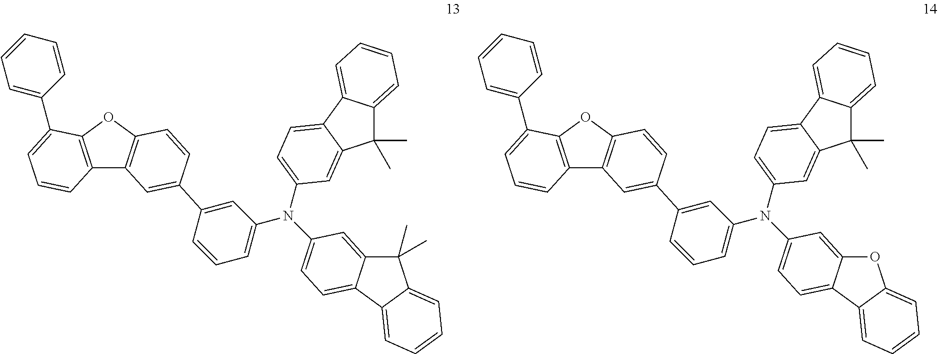 [SMILES: CC1(C)c2ccccc2-c2ccc(N(c3cccc(-c4ccc5oc6c(-c7ccccc7)cccc6c5c4)c3)c3ccc4c(c3)C(C)(C)c3ccccc3-4)cc21 ; CC1(C)c2ccccc2-c2ccc(N(c3cccc(-c4ccc5oc6c(-c7ccccc7)cccc6c5c4)c3)c3ccc4c(c3)oc3ccccc34)cc21]

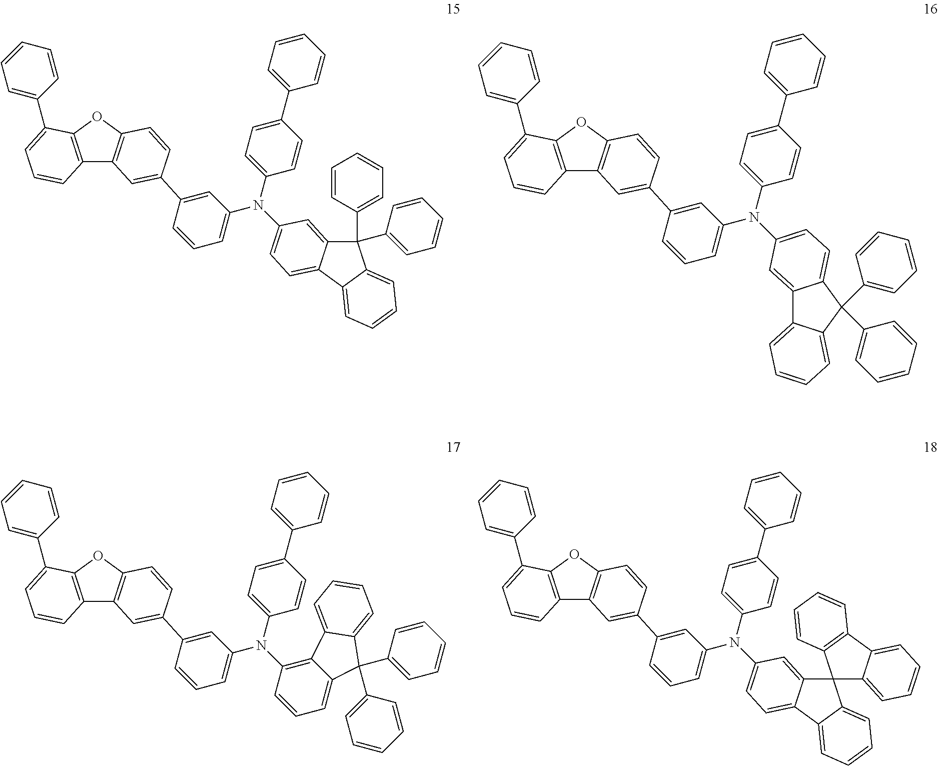 [SMILES: c1ccc(-c2ccc(N(c3cccc(-c4ccc5oc6c(-c7ccccc7)cccc6c5c4)c3)c3ccc4c(c3)C(c3ccccc3)(c3ccccc3)c3ccccc3-4)cc2)cc1 ; c1ccc(-c2ccc(N(c3cccc(-c4ccc5oc6c(-c7ccccc7)cccc6c5c4)c3)c3ccc4c(c3)-c3ccccc3C4(c3ccccc3)c3ccccc3)cc2)cc1 ; c1ccc(-c2ccc(N(c3cccc(-c4ccc5oc6c(-c7ccccc7)cccc6c5c4)c3)c3cccc4c3-c3ccccc3C4(c3ccccc3)c3ccccc3)cc2)cc1 ; c1ccc(-c2ccc(N(c3cccc(-c4ccc5oc6c(-c7ccccc7)cccc6c5c4)c3)c3ccc4c(c3)C3(c5ccccc5-c5ccccc53)c3ccccc3-4)cc2)cc1]

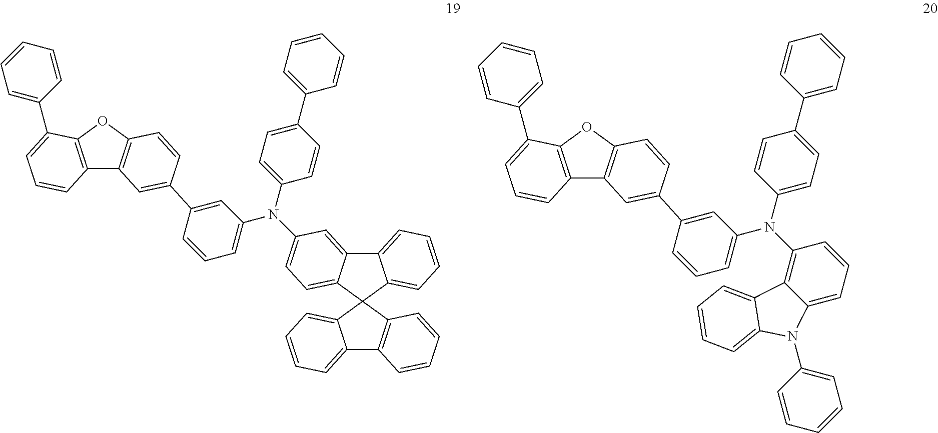 [SMILES: c1ccc(-c2ccc(N(c3cccc(-c4ccc5oc6c(-c7ccccc7)cccc6c5c4)c3)c3ccc4c(c3)-c3ccccc3C43c4ccccc4-c4ccccc43)cc2)cc1 ; c1ccc(-c2ccc(N(c3cccc(-c4ccc5oc6c(-c7ccccc7)cccc6c5c4)c3)c3cccc4c3c3ccccc3n4-c3ccccc3)cc2)cc1]

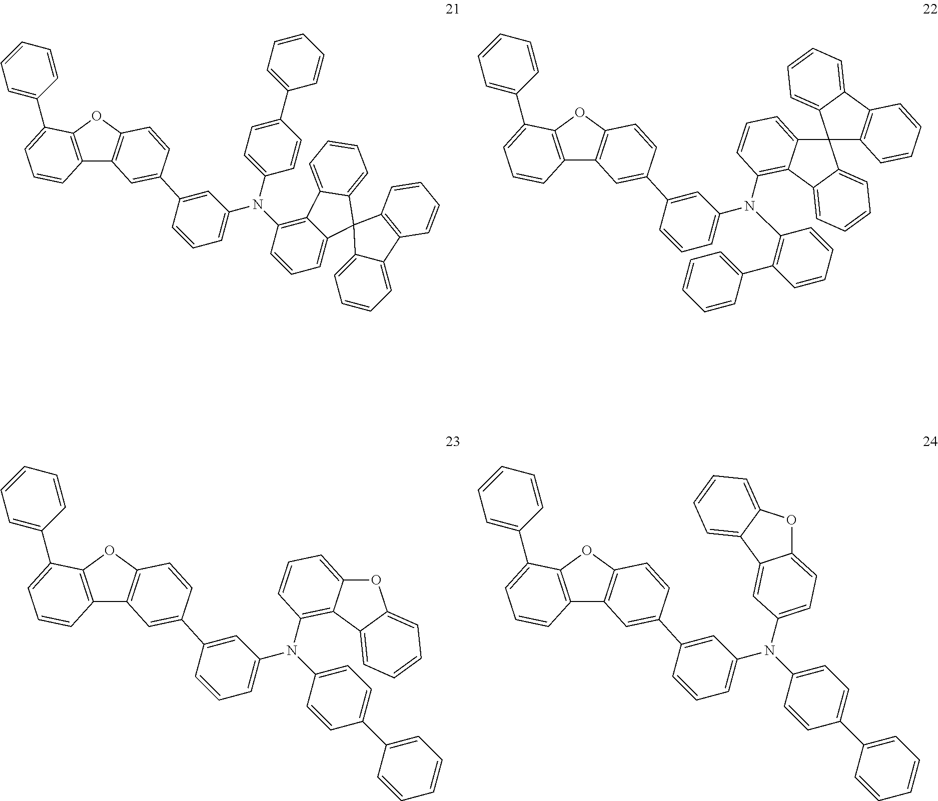 [SMILES: c1ccc(-c2ccc(N(c3cccc(-c4ccc5oc6c(-c7ccccc7)cccc6c5c4)c3)c3cccc4c3-c3ccccc3C43c4ccccc4-c4ccccc43)cc2)cc1 ; c1ccc(-c2ccccc2N(c2cccc(-c3ccc4oc5c(-c6ccccc6)cccc5c4c3)c2)c2cccc3c2-c2ccccc2C32c3ccccc3-c3ccccc32)cc1 ; c1ccc(-c2ccc(N(c3cccc(-c4ccc5oc6c(-c7ccccc7)cccc6c5c4)c3)c3cccc4oc5ccccc5c34)cc2)cc1 ; c1ccc(-c2ccc(N(c3cccc(-c4ccc5oc6c(-c7ccccc7)cccc6c5c4)c3)c3ccc4oc5ccccc5c4c3)cc2)cc1]

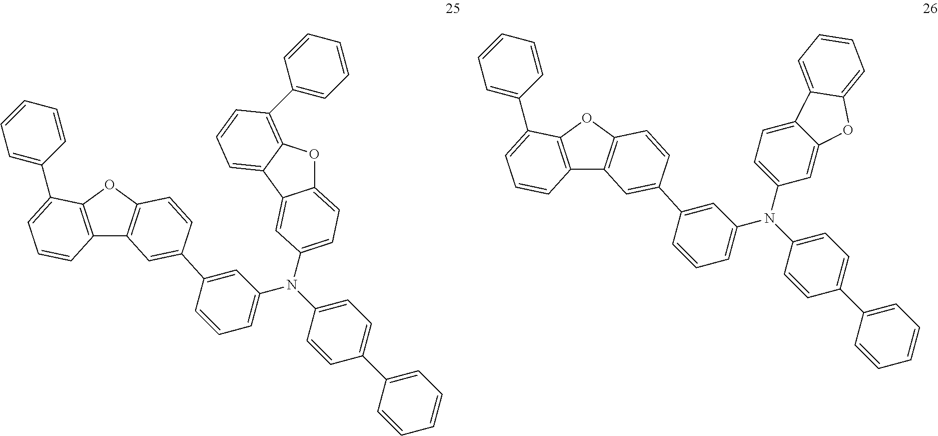 [SMILES: c1ccc(-c2ccc(N(c3cccc(-c4ccc5oc6c(-c7ccccc7)cccc6c5c4)c3)c3ccc4oc5c(-c6ccccc6)cccc5c4c3)cc2)cc1 ; c1ccc(-c2ccc(N(c3cccc(-c4ccc5oc6c(-c7ccccc7)cccc6c5c4)c3)c3ccc4c(c3)oc3ccccc34)cc2)cc1]

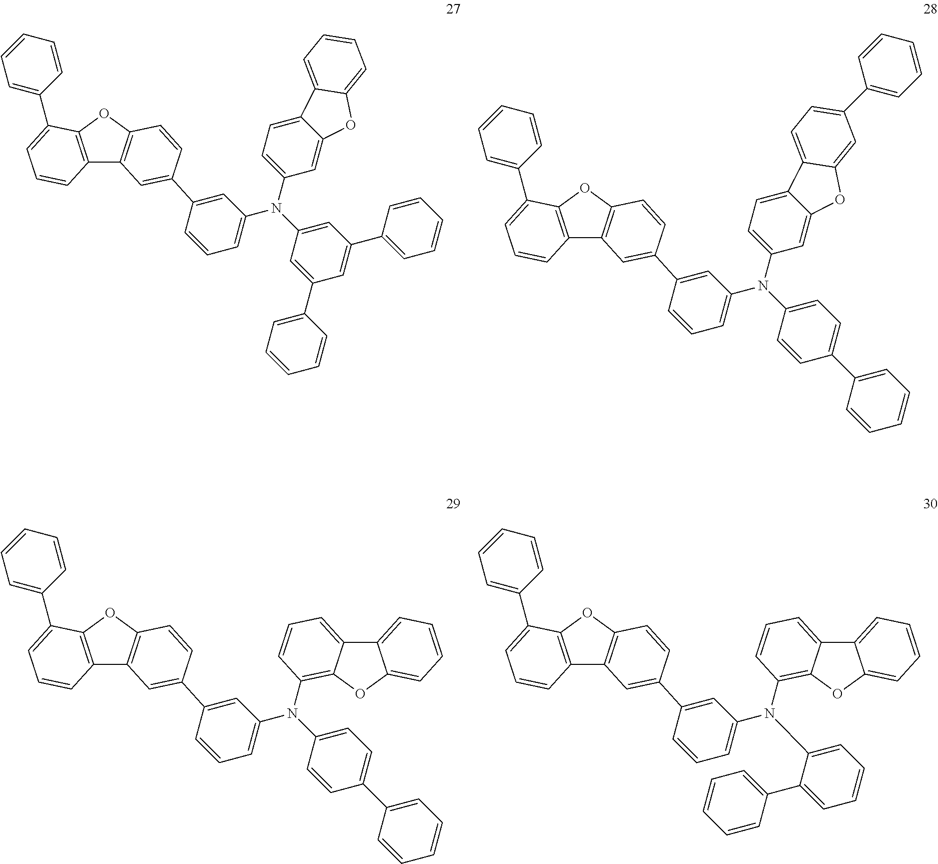 [SMILES: c1ccc(-c2cc(-c3ccccc3)cc(N(c3cccc(-c4ccc5oc6c(-c7ccccc7)cccc6c5c4)c3)c3ccc4c(c3)oc3ccccc34)c2)cc1 ; c1ccc(-c2ccc(N(c3cccc(-c4ccc5oc6c(-c7ccccc7)cccc6c5c4)c3)c3ccc4c(c3)oc3cc(-c5ccccc5)ccc34)cc2)cc1 ; c1ccc(-c2ccc(N(c3cccc(-c4ccc5oc6c(-c7ccccc7)cccc6c5c4)c3)c3cccc4c3oc3ccccc34)cc2)cc1 ; c1ccc(-c2ccccc2N(c2cccc(-c3ccc4oc5c(-c6ccccc6)cccc5c4c3)c2)c2cccc3c2oc2ccccc23)cc1]

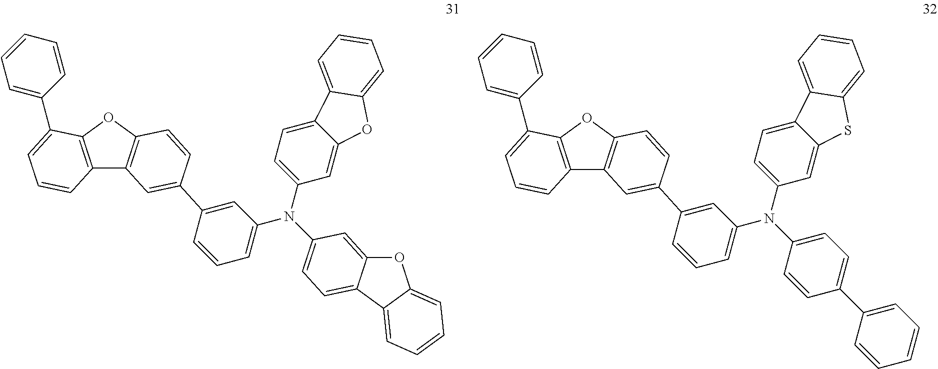 [SMILES: c1ccc(-c2cccc3c2oc2ccc(-c4cccc(N(c5ccc6c(c5)oc5ccccc56)c5ccc6c(c5)oc5ccccc56)c4)cc23)cc1 ; c1ccc(-c2ccc(N(c3cccc(-c4ccc5oc6c(-c7ccccc7)cccc6c5c4)c3)c3ccc4c(c3)sc3ccccc34)cc2)cc1]

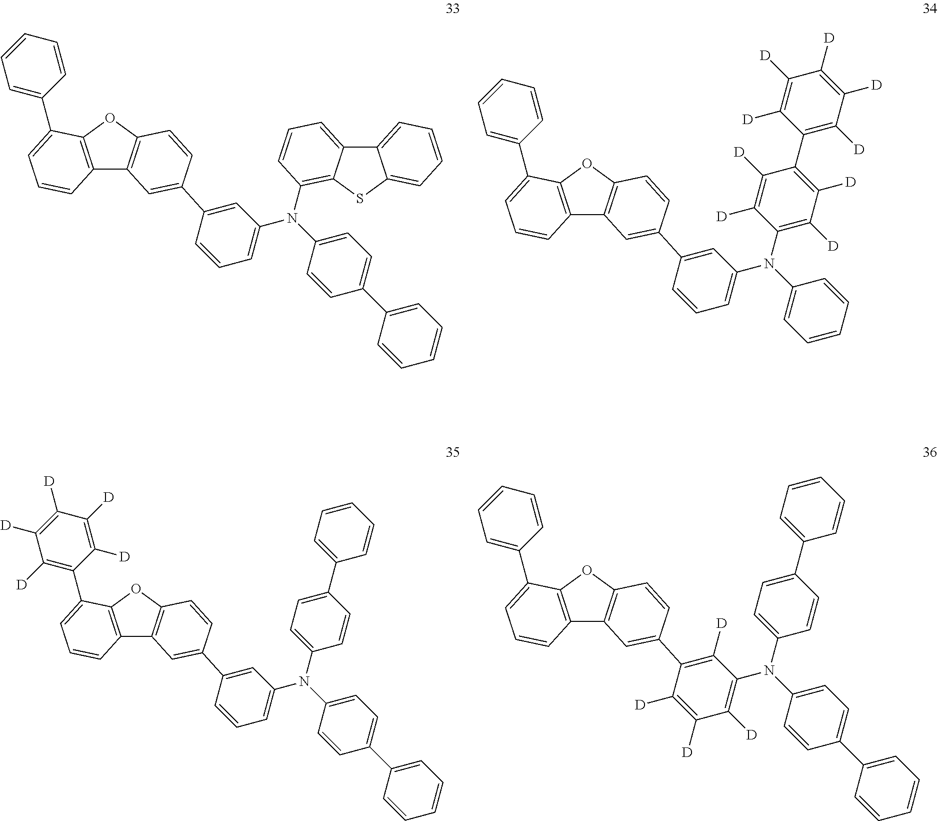 [SMILES: c1ccc(-c2ccc(N(c3cccc(-c4ccc5oc6c(-c7ccccc7)cccc6c5c4)c3)c3cccc4c3sc3ccccc34)cc2)cc1 ; [2H]c1c([2H])c([2H])c(-c2c([2H])c([2H])c(N(c3ccccc3)c3cccc(-c4ccc5oc6c(-c7ccccc7)cccc6c5c4)c3)c([2H])c2[2H])c([2H])c1[2H] ; [2H]c1c([2H])c([2H])c(-c2cccc3c2oc2ccc(-c4cccc(N(c5ccc(-c6ccccc6)cc5)c5ccc(-c6ccccc6)cc5)c4)cc23)c([2H])c1[2H] ; [2H]c1c([2H])c(-c2ccc3oc4c(-c5ccccc5)cccc4c3c2)c([2H])c(N(c2ccc(-c3ccccc3)cc2)c2ccc(-c3ccccc3)cc2)c1[2H]]

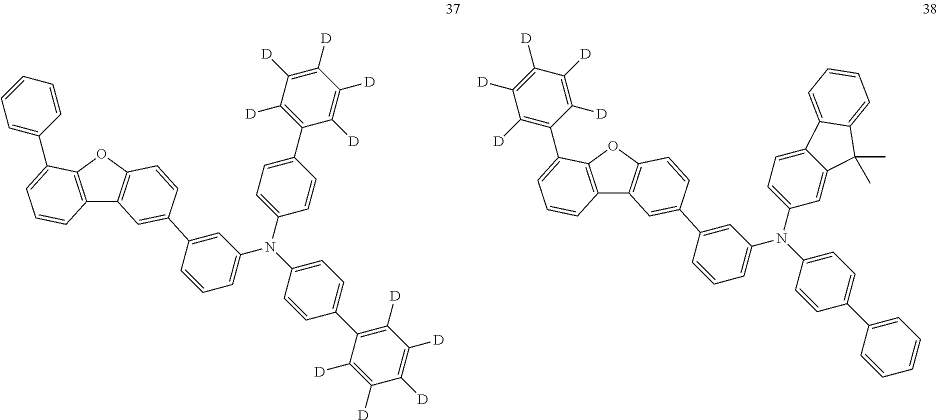 [SMILES: [2H]c1c([2H])c([2H])c(-c2ccc(N(c3ccc(-c4c([2H])c([2H])c([2H])c([2H])c4[2H])cc3)c3cccc(-c4ccc5oc6c(-c7ccccc7)cccc6c5c4)c3)cc2)c([2H])c1[2H] ; [2H]c1c([2H])c([2H])c(-c2cccc3c2oc2ccc(-c4cccc(N(c5ccc(-c6ccccc6)cc5)c5ccc6c(c5)C(C)(C)c5ccccc5-6)c4)cc23)c([2H])c1[2H]]

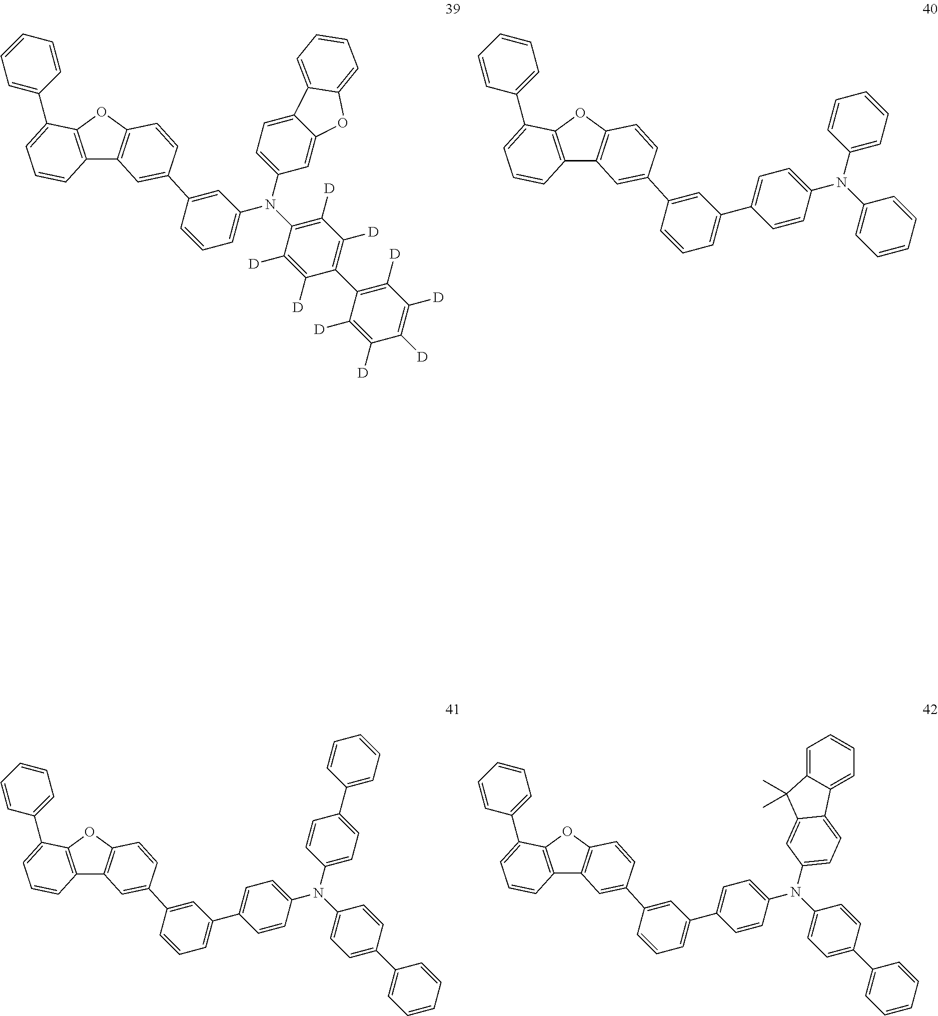 [SMILES: [2H]c1c([2H])c([2H])c(-c2c([2H])c([2H])c(N(c3cccc(-c4ccc5oc6c(-c7ccccc7)cccc6c5c4)c3)c3ccc4c(c3)oc3ccccc34)c([2H])c2[2H])c([2H])c1[2H] ; c1ccc(-c2cccc3c2oc2ccc(-c4cccc(-c5ccc(N(c6ccccc6)c6ccccc6)cc5)c4)cc23)cc1 ; c1ccc(-c2ccc(N(c3ccc(-c4ccccc4)cc3)c3ccc(-c4cccc(-c5ccc6oc7c(-c8ccccc8)cccc7c6c5)c4)cc3)cc2)cc1 ; CC1(C)c2ccccc2-c2ccc(N(c3ccc(-c4ccccc4)cc3)c3ccc(-c4cccc(-c5ccc6oc7c(-c8ccccc8)cccc7c6c5)c4)cc3)cc21]

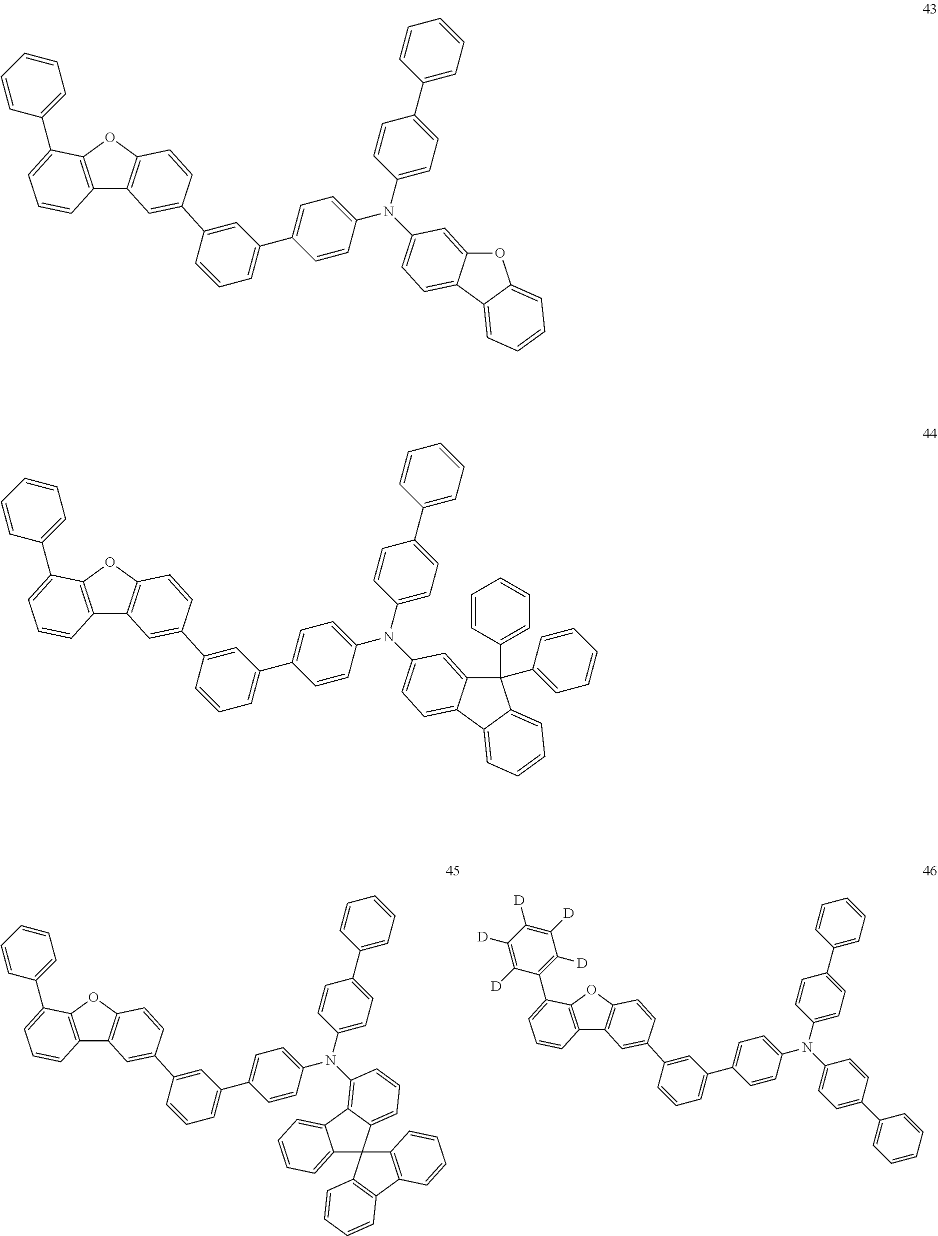 [SMILES: c1ccc(-c2ccc(N(c3ccc(-c4cccc(-c5ccc6oc7c(-c8ccccc8)cccc7c6c5)c4)cc3)c3ccc4c(c3)oc3ccccc34)cc2)cc1 ; c1ccc(-c2ccc(N(c3ccc(-c4cccc(-c5ccc6oc7c(-c8ccccc8)cccc7c6c5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6oc7c(-c8ccccc8)cccc7c6c5)c4)cc3)c3cccc4c3-c3ccccc3C43c4ccccc4-c4ccccc43)cc2)cc1 ; [2H]c1c([2H])c([2H])c(-c2cccc3c2oc2ccc(-c4cccc(-c5ccc(N(c6ccc(-c7ccccc7)cc6)c6ccc(-c7ccccc7)cc6)cc5)c4)cc23)c([2H])c1[2H]]

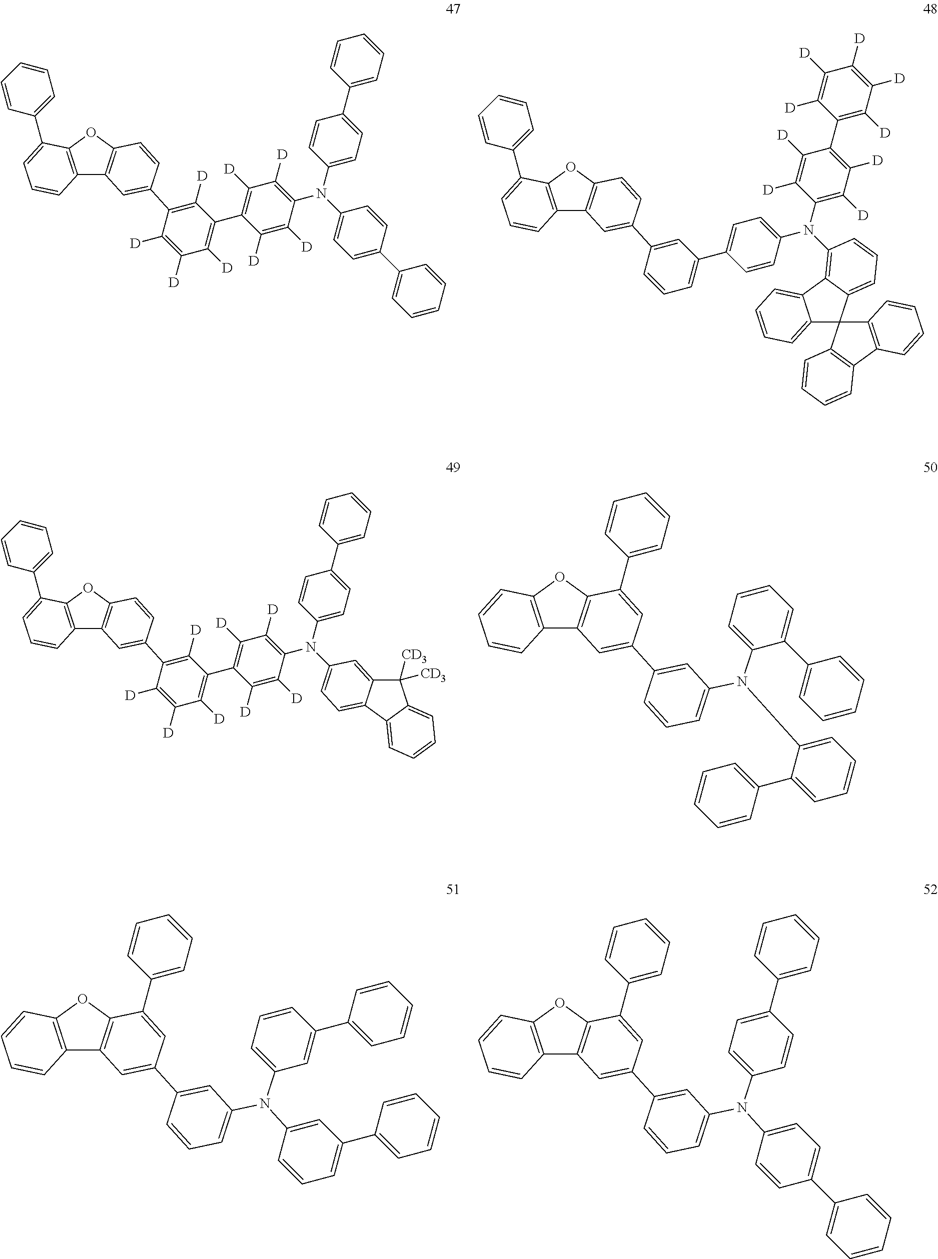

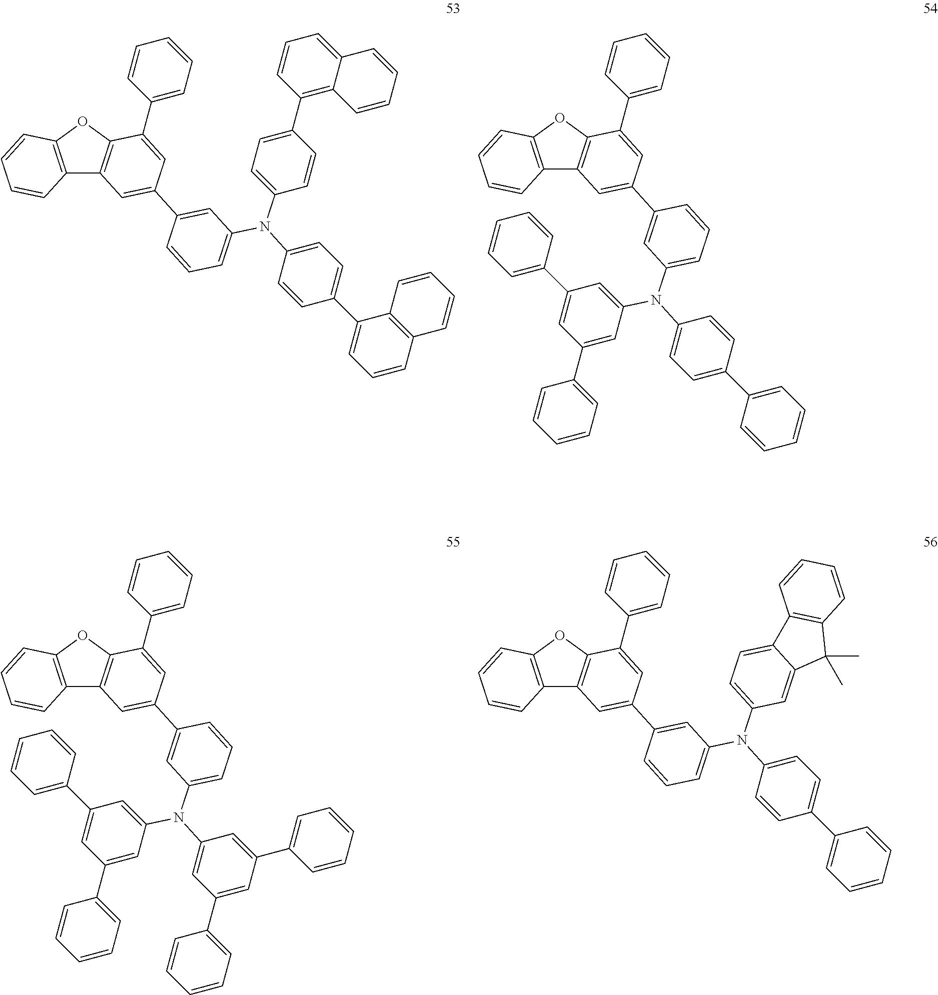 [SMILES: c1ccc(-c2cc(-c3cccc(N(c4ccc(-c5cccc6ccccc56)cc4)c4ccc(-c5cccc6ccccc56)cc4)c3)cc3c2oc2ccccc23)cc1 ; c1ccc(-c2ccc(N(c3cccc(-c4cc(-c5ccccc5)c5oc6ccccc6c5c4)c3)c3cc(-c4ccccc4)cc(-c4ccccc4)c3)cc2)cc1 ; c1ccc(-c2cc(-c3ccccc3)cc(N(c3cccc(-c4cc(-c5ccccc5)c5oc6ccccc6c5c4)c3)c3cc(-c4ccccc4)cc(-c4ccccc4)c3)c2)cc1 ; CC1(C)c2ccccc2-c2ccc(N(c3ccc(-c4ccccc4)cc3)c3cccc(-c4cc(-c5ccccc5)c5oc6ccccc6c5c4)c3)cc21]

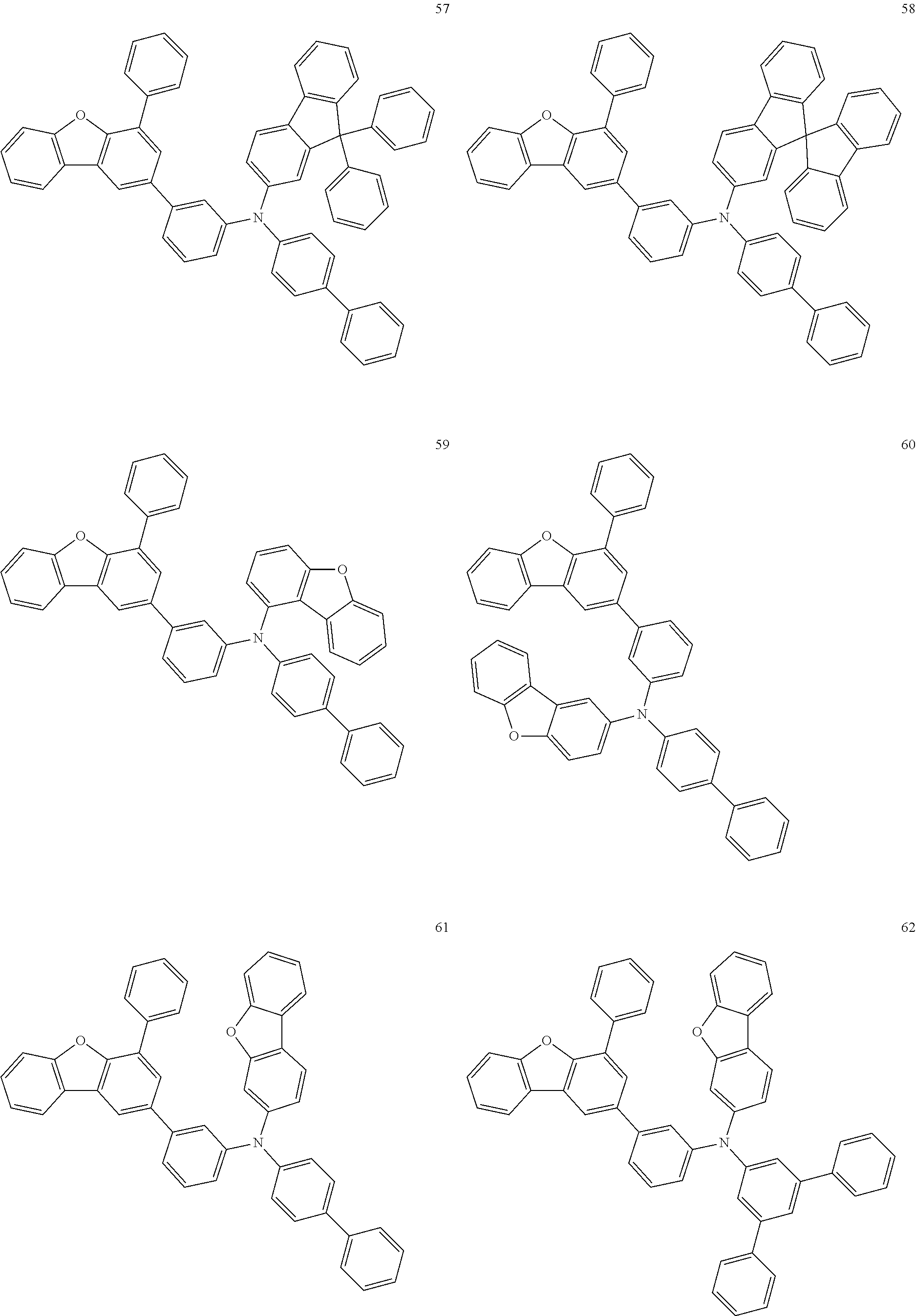

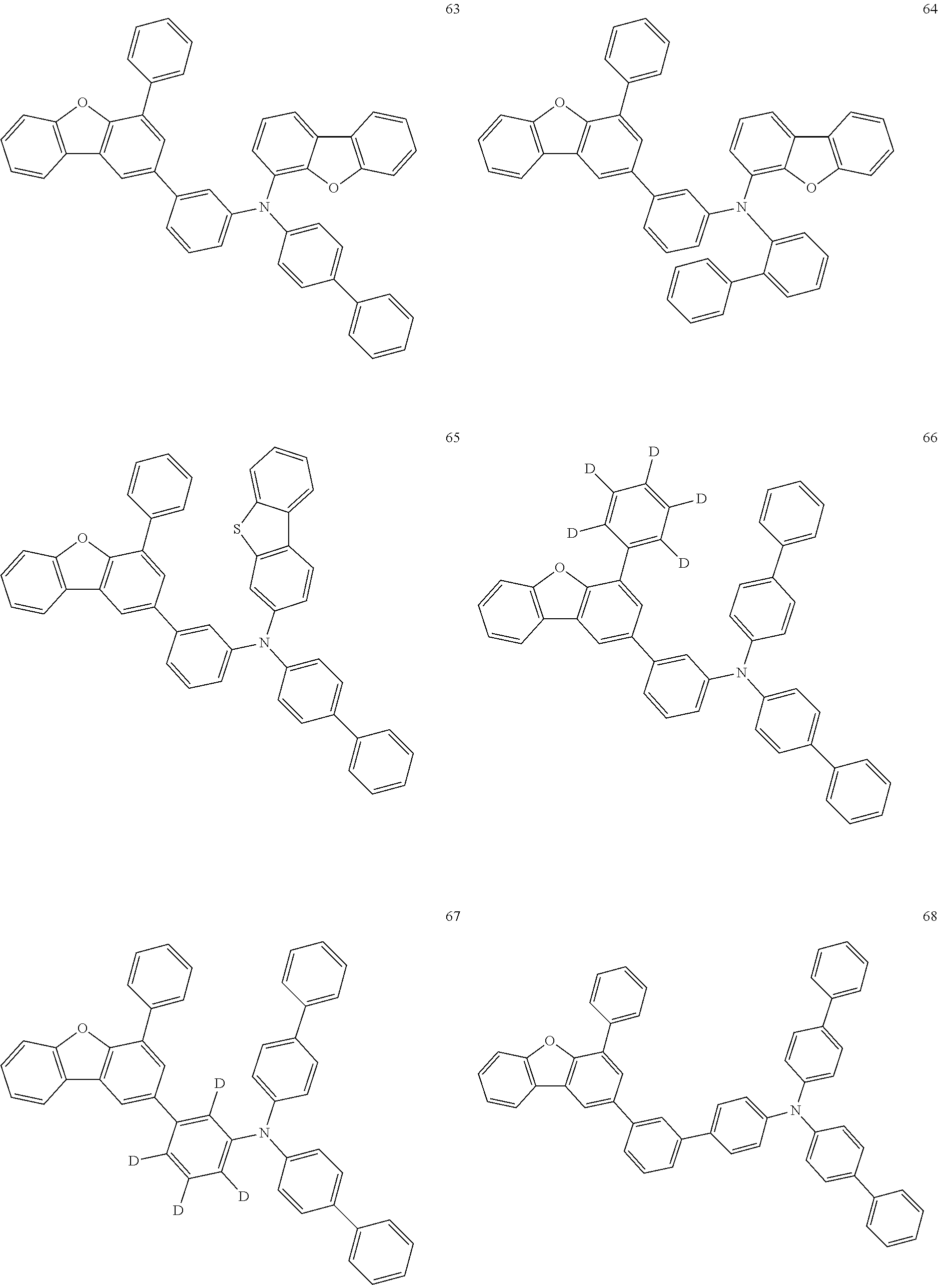

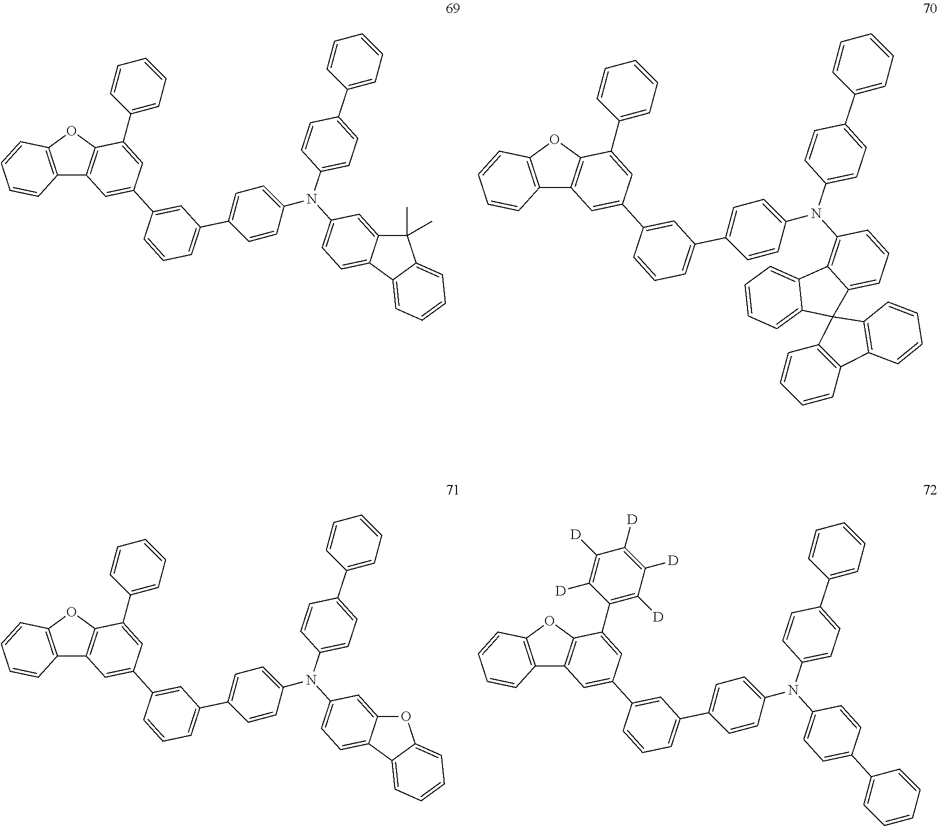 [SMILES: CC1(C)c2ccccc2-c2ccc(N(c3ccc(-c4ccccc4)cc3)c3ccc(-c4cccc(-c5cc(-c6ccccc6)c6oc7ccccc7c6c5)c4)cc3)cc21 ; c1ccc(-c2ccc(N(c3ccc(-c4cccc(-c5cc(-c6ccccc6)c6oc7ccccc7c6c5)c4)cc3)c3cccc4c3-c3ccccc3C43c4ccccc4-c4ccccc43)cc2)cc1 ; c1ccc(-c2ccc(N(c3ccc(-c4cccc(-c5cc(-c6ccccc6)c6oc7ccccc7c6c5)c4)cc3)c3ccc4c(c3)oc3ccccc34)cc2)cc1 ; [2H]c1c([2H])c([2H])c(-c2cc(-c3cccc(-c4ccc(N(c5ccc(-c6ccccc6)cc5)c5ccc(-c6ccccc6)cc5)cc4)c3)cc3c2oc2ccccc23)c([2H])c1[2H]]

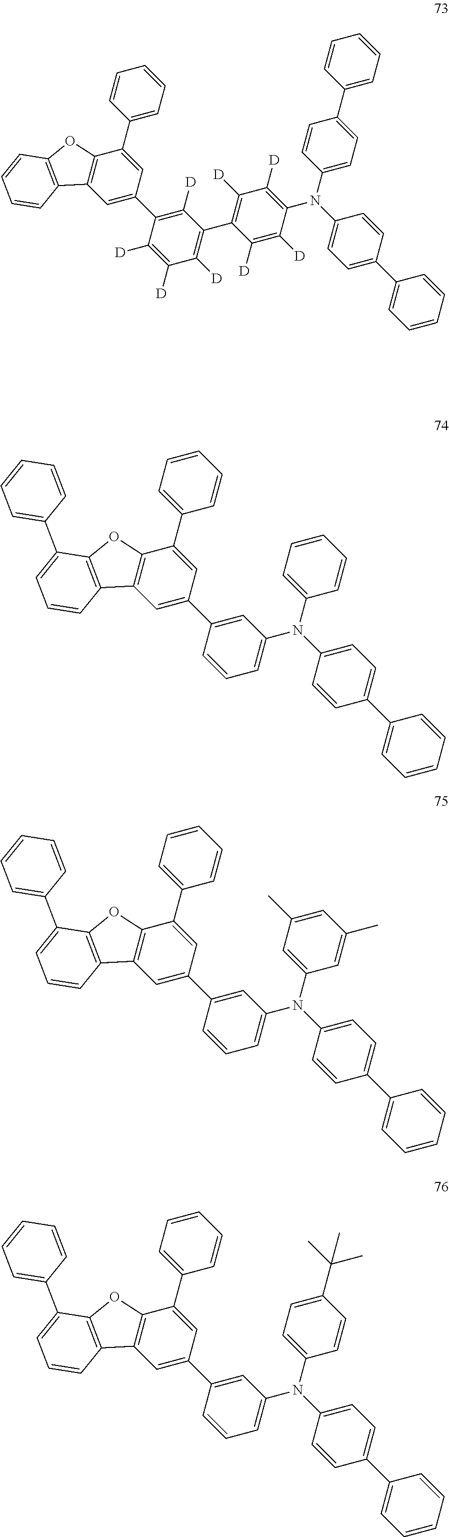 [SMILES: [2H]c1c([2H])c(-c2cc(-c3ccccc3)c3oc4ccccc4c3c2)c([2H])c(-c2c([2H])c([2H])c(N(c3ccc(-c4ccccc4)cc3)c3ccc(-c4ccccc4)cc3)c([2H])c2[2H])c1[2H] ; c1ccc(-c2ccc(N(c3ccccc3)c3cccc(-c4cc(-c5ccccc5)c5oc6c(-c7ccccc7)cccc6c5c4)c3)cc2)cc1 ; Cc1cc(C)cc(N(c2ccc(-c3ccccc3)cc2)c2cccc(-c3cc(-c4ccccc4)c4oc5c(-c6ccccc6)cccc5c4c3)c2)c1 ; CC(C)(C)c1ccc(N(c2ccc(-c3ccccc3)cc2)c2cccc(-c3cc(-c4ccccc4)c4oc5c(-c6ccccc6)cccc5c4c3)c2)cc1]

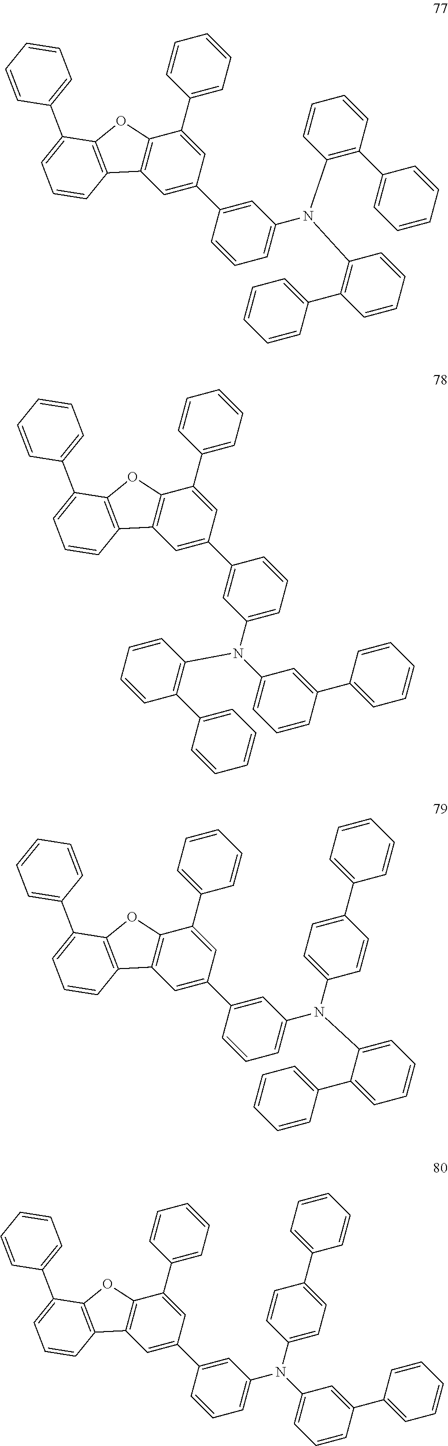 [SMILES: c1ccc(-c2ccccc2N(c2cccc(-c3cc(-c4ccccc4)c4oc5c(-c6ccccc6)cccc5c4c3)c2)c2ccccc2-c2ccccc2)cc1 ; c1ccc(-c2cccc(N(c3cccc(-c4cc(-c5ccccc5)c5oc6c(-c7ccccc7)cccc6c5c4)c3)c3ccccc3-c3ccccc3)c2)cc1 ; c1ccc(-c2ccc(N(c3cccc(-c4cc(-c5ccccc5)c5oc6c(-c7ccccc7)cccc6c5c4)c3)c3ccccc3-c3ccccc3)cc2)cc1 ; c1ccc(-c2ccc(N(c3cccc(-c4ccccc4)c3)c3cccc(-c4cc(-c5ccccc5)c5oc6c(-c7ccccc7)cccc6c5c4)c3)cc2)cc1]

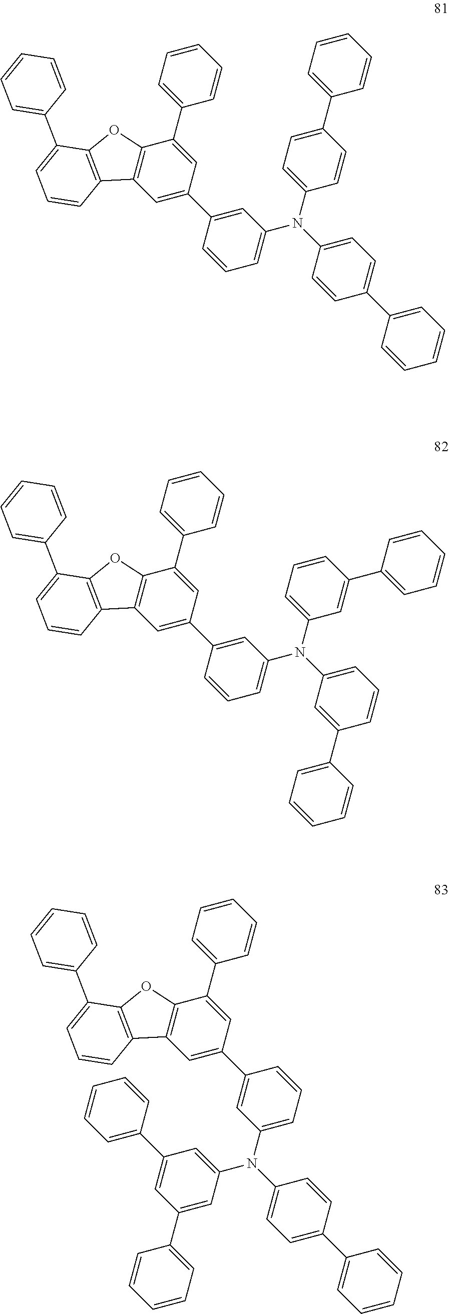 [SMILES: c1ccc(-c2ccc(N(c3ccc(-c4ccccc4)cc3)c3cccc(-c4cc(-c5ccccc5)c5oc6c(-c7ccccc7)cccc6c5c4)c3)cc2)cc1 ; c1ccc(-c2cccc(N(c3cccc(-c4ccccc4)c3)c3cccc(-c4cc(-c5ccccc5)c5oc6c(-c7ccccc7)cccc6c5c4)c3)c2)cc1 ; c1ccc(-c2ccc(N(c3cccc(-c4cc(-c5ccccc5)c5oc6c(-c7ccccc7)cccc6c5c4)c3)c3cc(-c4ccccc4)cc(-c4ccccc4)c3)cc2)cc1]

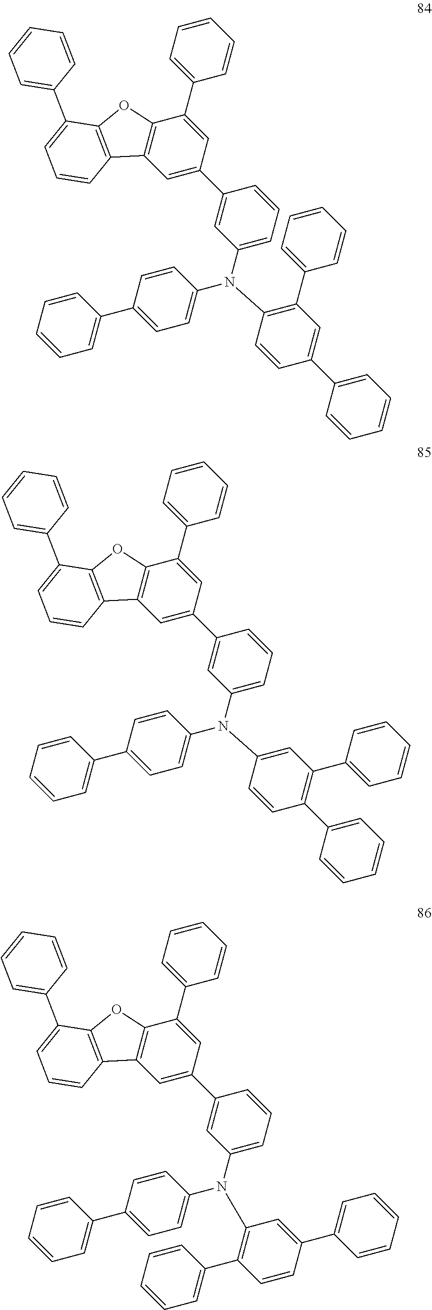 [SMILES: c1ccc(-c2ccc(N(c3cccc(-c4cc(-c5ccccc5)c5oc6c(-c7ccccc7)cccc6c5c4)c3)c3ccc(-c4ccccc4)cc3-c3ccccc3)cc2)cc1 ; c1ccc(-c2ccc(N(c3cccc(-c4cc(-c5ccccc5)c5oc6c(-c7ccccc7)cccc6c5c4)c3)c3ccc(-c4ccccc4)c(-c4ccccc4)c3)cc2)cc1 ; c1ccc(-c2ccc(N(c3cccc(-c4cc(-c5ccccc5)c5oc6c(-c7ccccc7)cccc6c5c4)c3)c3cc(-c4ccccc4)ccc3-c3ccccc3)cc2)cc1]

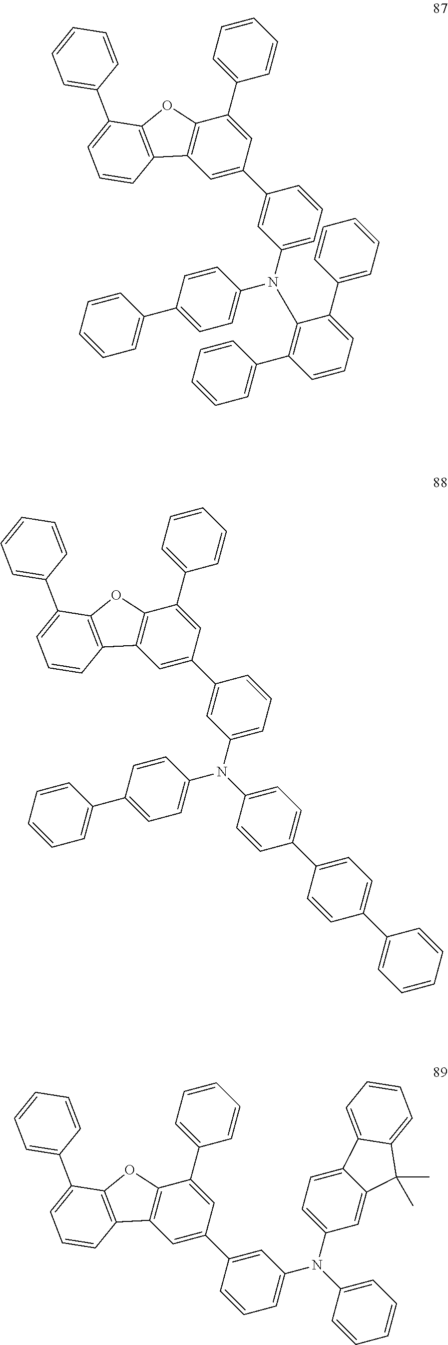 [SMILES: c1ccc(-c2ccc(N(c3cccc(-c4cc(-c5ccccc5)c5oc6c(-c7ccccc7)cccc6c5c4)c3)c3c(-c4ccccc4)cccc3-c3ccccc3)cc2)cc1 ; c1ccc(-c2ccc(-c3ccc(N(c4ccc(-c5ccccc5)cc4)c4cccc(-c5cc(-c6ccccc6)c6oc7c(-c8ccccc8)cccc7c6c5)c4)cc3)cc2)cc1 ; CC1(C)c2ccccc2-c2ccc(N(c3ccccc3)c3cccc(-c4cc(-c5ccccc5)c5oc6c(-c7ccccc7)cccc6c5c4)c3)cc21]

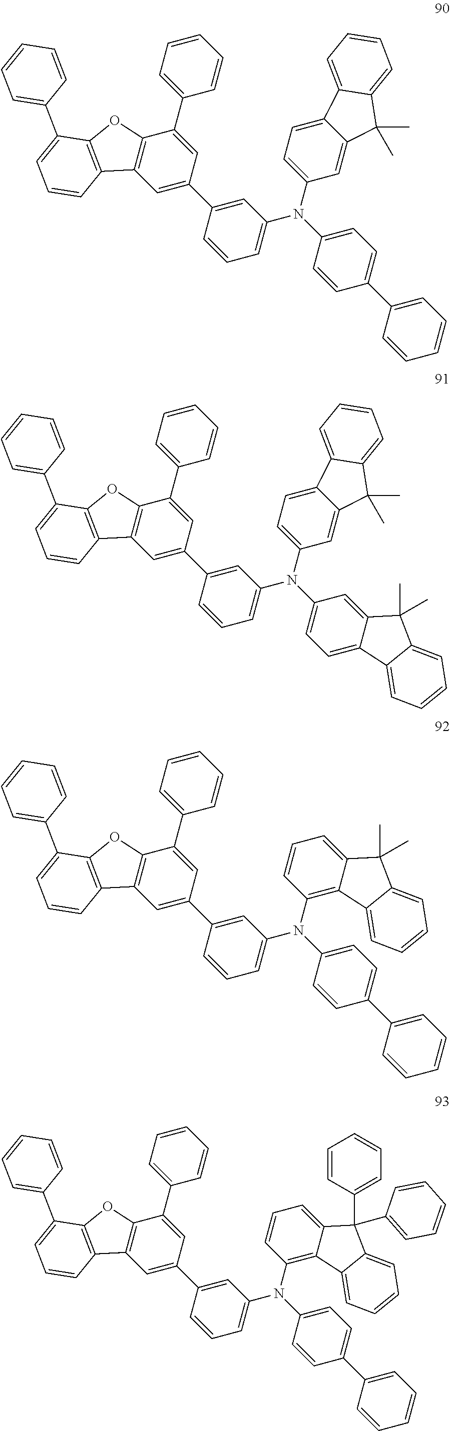 [SMILES: CC1(C)c2ccccc2-c2ccc(N(c3ccc(-c4ccccc4)cc3)c3cccc(-c4cc(-c5ccccc5)c5oc6c(-c7ccccc7)cccc6c5c4)c3)cc21 ; CC1(C)c2ccccc2-c2ccc(N(c3cccc(-c4cc(-c5ccccc5)c5oc6c(-c7ccccc7)cccc6c5c4)c3)c3ccc4c(c3)C(C)(C)c3ccccc3-4)cc21 ; CC1(C)c2ccccc2-c2c(N(c3ccc(-c4ccccc4)cc3)c3cccc(-c4cc(-c5ccccc5)c5oc6c(-c7ccccc7)cccc6c5c4)c3)cccc21 ; c1ccc(-c2ccc(N(c3cccc(-c4cc(-c5ccccc5)c5oc6c(-c7ccccc7)cccc6c5c4)c3)c3cccc4c3-c3ccccc3C4(c3ccccc3)c3ccccc3)cc2)cc1]

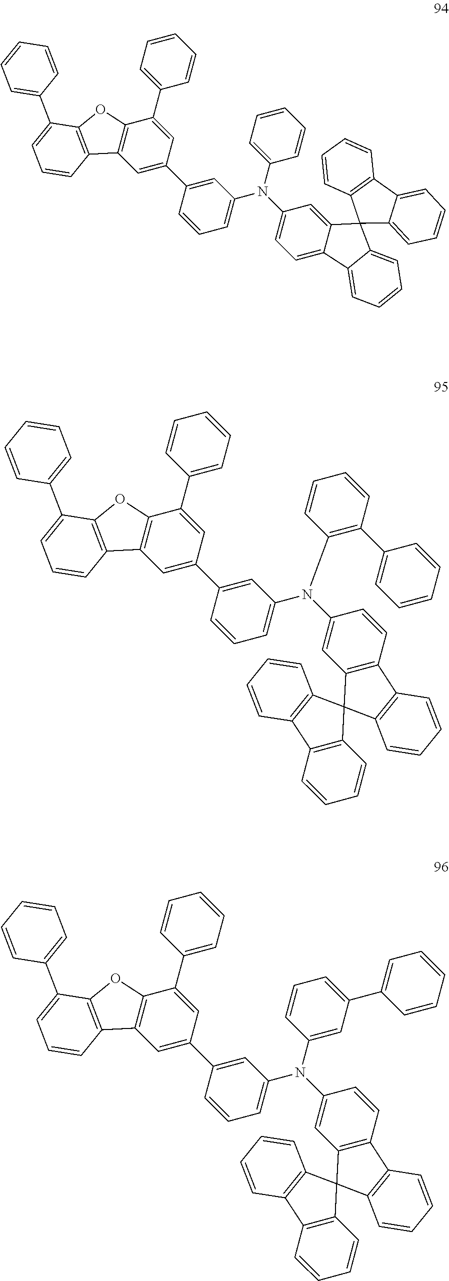 [SMILES: c1ccc(-c2cccc3c2oc2c(-c4ccccc4)cc(-c4cccc(N(c5ccccc5)c5ccc6c(c5)C5(c7ccccc7-c7ccccc75)c5ccccc5-6)c4)cc23)cc1 ; c1ccc(-c2ccccc2N(c2cccc(-c3cc(-c4ccccc4)c4oc5c(-c6ccccc6)cccc5c4c3)c2)c2ccc3c(c2)C2(c4ccccc4-c4ccccc42)c2ccccc2-3)cc1 ; c1ccc(-c2cccc(N(c3cccc(-c4cc(-c5ccccc5)c5oc6c(-c7ccccc7)cccc6c5c4)c3)c3ccc4c(c3)C3(c5ccccc5-c5ccccc53)c3ccccc3-4)c2)cc1]

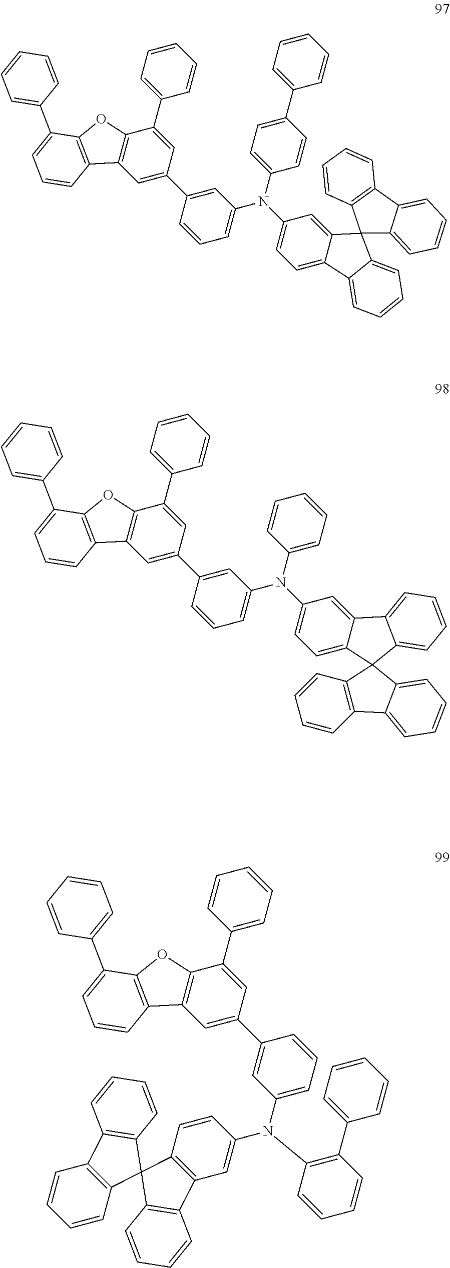 [SMILES: c1ccc(-c2ccc(N(c3cccc(-c4cc(-c5ccccc5)c5oc6c(-c7ccccc7)cccc6c5c4)c3)c3ccc4c(c3)C3(c5ccccc5-c5ccccc53)c3ccccc3-4)cc2)cc1 ; c1ccc(-c2cccc3c2oc2c(-c4ccccc4)cc(-c4cccc(N(c5ccccc5)c5ccc6c(c5)-c5ccccc5C65c6ccccc6-c6ccccc65)c4)cc23)cc1 ; c1ccc(-c2ccccc2N(c2cccc(-c3cc(-c4ccccc4)c4oc5c(-c6ccccc6)cccc5c4c3)c2)c2ccc3c(c2)-c2ccccc2C32c3ccccc3-c3ccccc32)cc1]

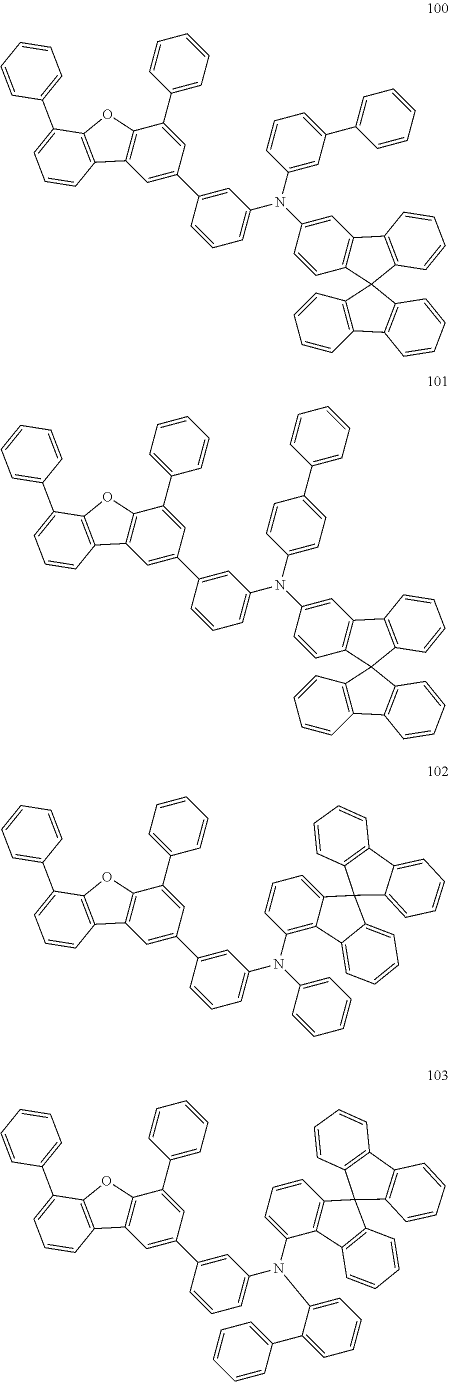 [SMILES: c1ccc(-c2cccc(N(c3cccc(-c4cc(-c5ccccc5)c5oc6c(-c7ccccc7)cccc6c5c4)c3)c3ccc4c(c3)-c3ccccc3C43c4ccccc4-c4ccccc43)c2)cc1 ; c1ccc(-c2ccc(N(c3cccc(-c4cc(-c5ccccc5)c5oc6c(-c7ccccc7)cccc6c5c4)c3)c3ccc4c(c3)-c3ccccc3C43c4ccccc4-c4ccccc43)cc2)cc1 ; c1ccc(-c2cccc3c2oc2c(-c4ccccc4)cc(-c4cccc(N(c5ccccc5)c5cccc6c5-c5ccccc5C65c6ccccc6-c6ccccc65)c4)cc23)cc1 ; c1ccc(-c2ccccc2N(c2cccc(-c3cc(-c4ccccc4)c4oc5c(-c6ccccc6)cccc5c4c3)c2)c2cccc3c2-c2ccccc2C32c3ccccc3-c3ccccc32)cc1]

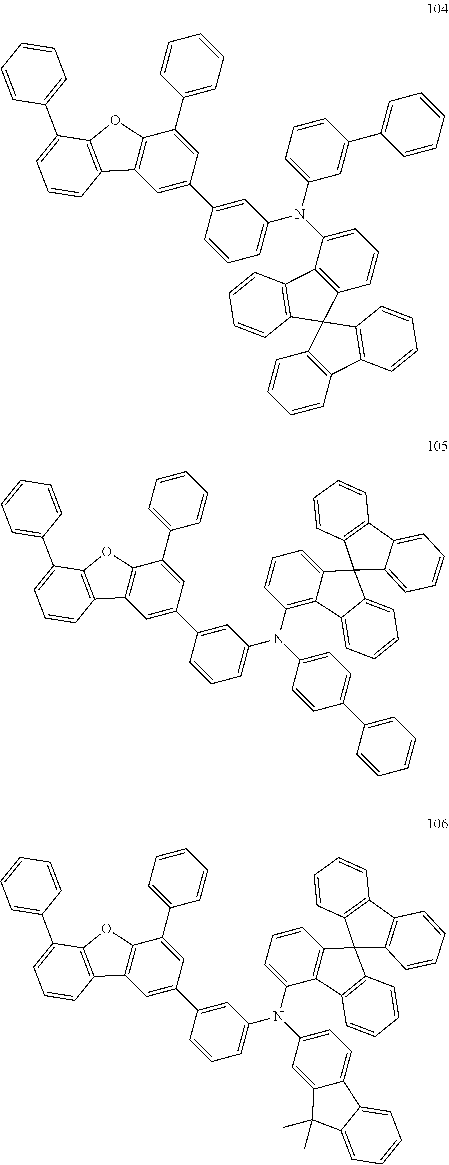 [SMILES: c1ccc(-c2cccc(N(c3cccc(-c4cc(-c5ccccc5)c5oc6c(-c7ccccc7)cccc6c5c4)c3)c3cccc4c3-c3ccccc3C43c4ccccc4-c4ccccc43)c2)cc1 ; c1ccc(-c2ccc(N(c3cccc(-c4cc(-c5ccccc5)c5oc6c(-c7ccccc7)cccc6c5c4)c3)c3cccc4c3-c3ccccc3C43c4ccccc4-c4ccccc43)cc2)cc1 ; CC1(C)c2ccccc2-c2ccc(N(c3cccc(-c4cc(-c5ccccc5)c5oc6c(-c7ccccc7)cccc6c5c4)c3)c3cccc4c3-c3ccccc3C43c4ccccc4-c4ccccc43)cc21]

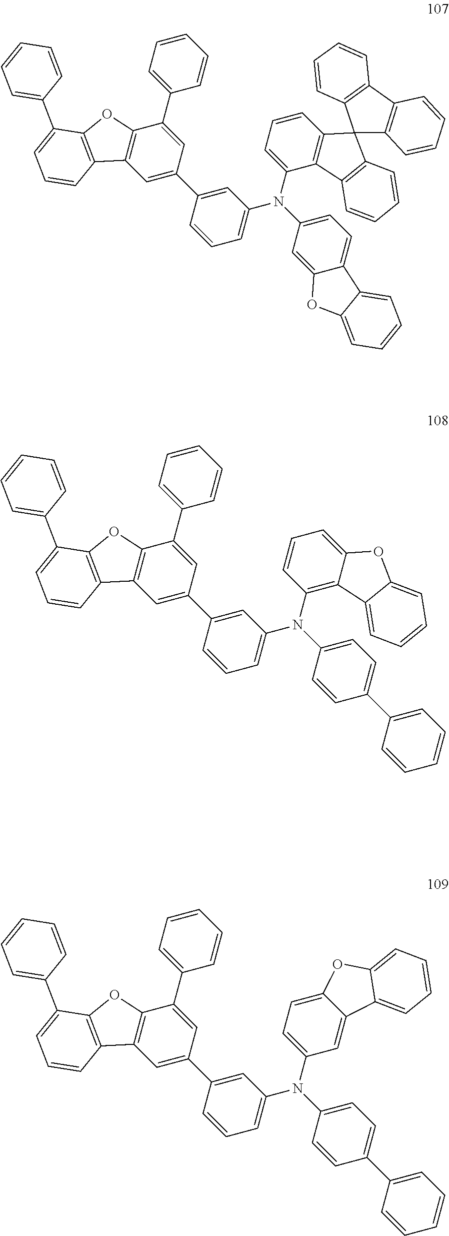 [SMILES: c1ccc(-c2cccc3c2oc2c(-c4ccccc4)cc(-c4cccc(N(c5ccc6c(c5)oc5ccccc56)c5cccc6c5-c5ccccc5C65c6ccccc6-c6ccccc65)c4)cc23)cc1 ; c1ccc(-c2ccc(N(c3cccc(-c4cc(-c5ccccc5)c5oc6c(-c7ccccc7)cccc6c5c4)c3)c3cccc4oc5ccccc5c34)cc2)cc1 ; c1ccc(-c2ccc(N(c3cccc(-c4cc(-c5ccccc5)c5oc6c(-c7ccccc7)cccc6c5c4)c3)c3ccc4oc5ccccc5c4c3)cc2)cc1]

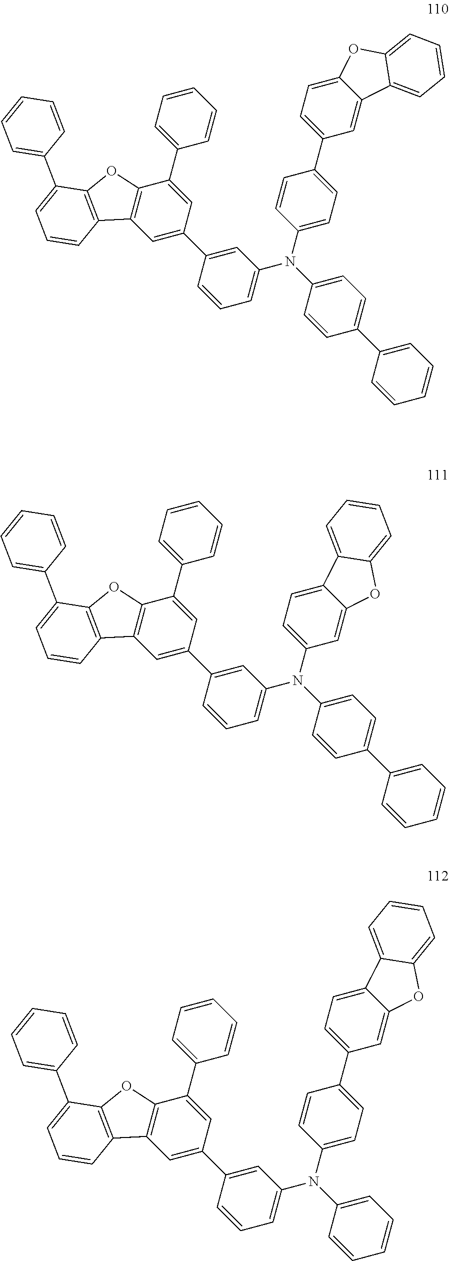 [SMILES: c1ccc(-c2ccc(N(c3ccc(-c4ccc5oc6ccccc6c5c4)cc3)c3cccc(-c4cc(-c5ccccc5)c5oc6c(-c7ccccc7)cccc6c5c4)c3)cc2)cc1 ; c1ccc(-c2ccc(N(c3cccc(-c4cc(-c5ccccc5)c5oc6c(-c7ccccc7)cccc6c5c4)c3)c3ccc4c(c3)oc3ccccc34)cc2)cc1 ; c1ccc(-c2cccc3c2oc2c(-c4ccccc4)cc(-c4cccc(N(c5ccccc5)c5ccc(-c6ccc7c(c6)oc6ccccc67)cc5)c4)cc23)cc1]

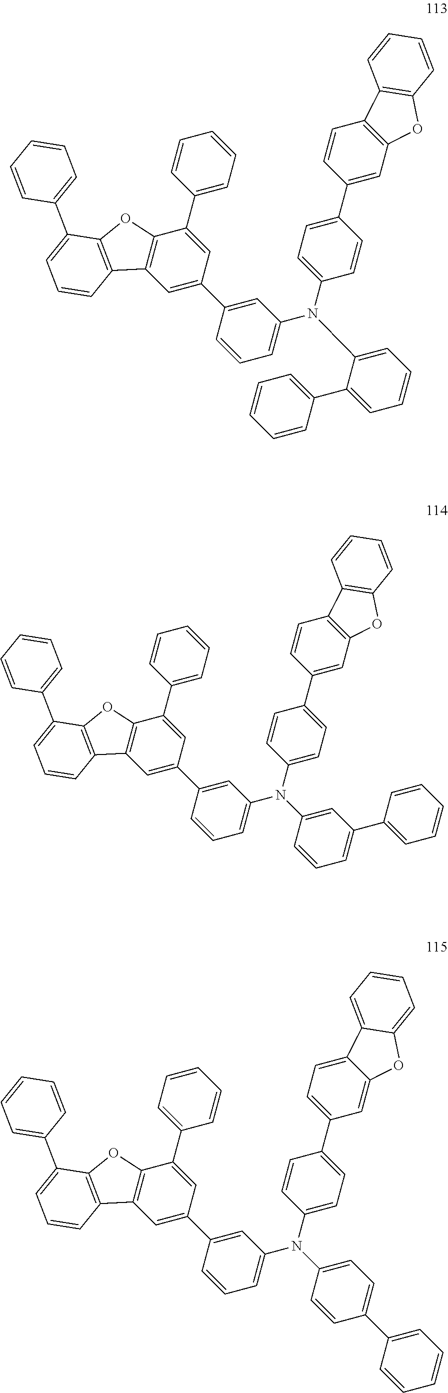 [SMILES: c1ccc(-c2ccccc2N(c2ccc(-c3ccc4c(c3)oc3ccccc34)cc2)c2cccc(-c3cc(-c4ccccc4)c4oc5c(-c6ccccc6)cccc5c4c3)c2)cc1 ; c1ccc(-c2cccc(N(c3ccc(-c4ccc5c(c4)oc4ccccc45)cc3)c3cccc(-c4cc(-c5ccccc5)c5oc6c(-c7ccccc7)cccc6c5c4)c3)c2)cc1 ; c1ccc(-c2ccc(N(c3ccc(-c4ccc5c(c4)oc4ccccc45)cc3)c3cccc(-c4cc(-c5ccccc5)c5oc6c(-c7ccccc7)cccc6c5c4)c3)cc2)cc1]

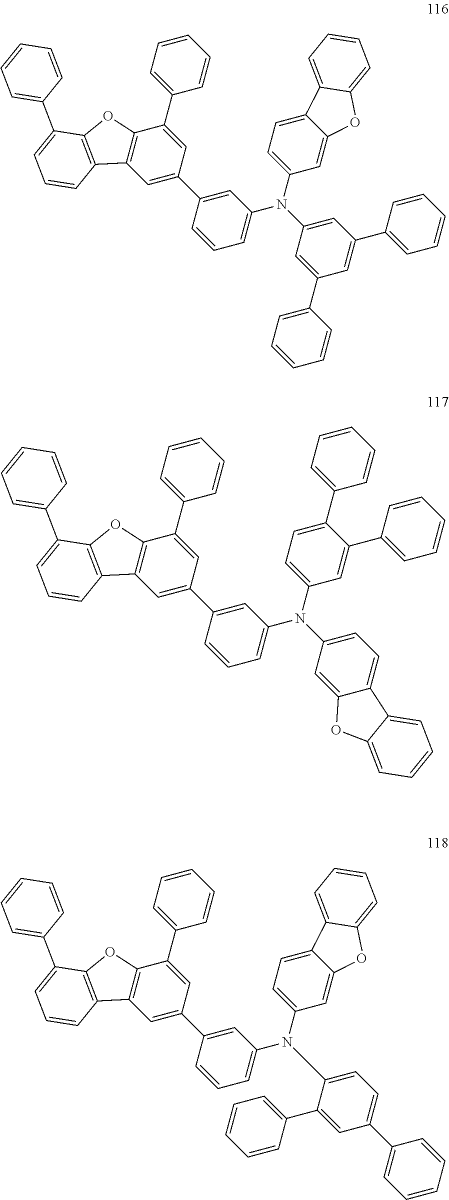 [SMILES: c1ccc(-c2cc(-c3ccccc3)cc(N(c3cccc(-c4cc(-c5ccccc5)c5oc6c(-c7ccccc7)cccc6c5c4)c3)c3ccc4c(c3)oc3ccccc34)c2)cc1 ; c1ccc(-c2ccc(N(c3cccc(-c4cc(-c5ccccc5)c5oc6c(-c7ccccc7)cccc6c5c4)c3)c3ccc4c(c3)oc3ccccc34)cc2-c2ccccc2)cc1 ; c1ccc(-c2ccc(N(c3cccc(-c4cc(-c5ccccc5)c5oc6c(-c7ccccc7)cccc6c5c4)c3)c3ccc4c(c3)oc3ccccc34)c(-c3ccccc3)c2)cc1]

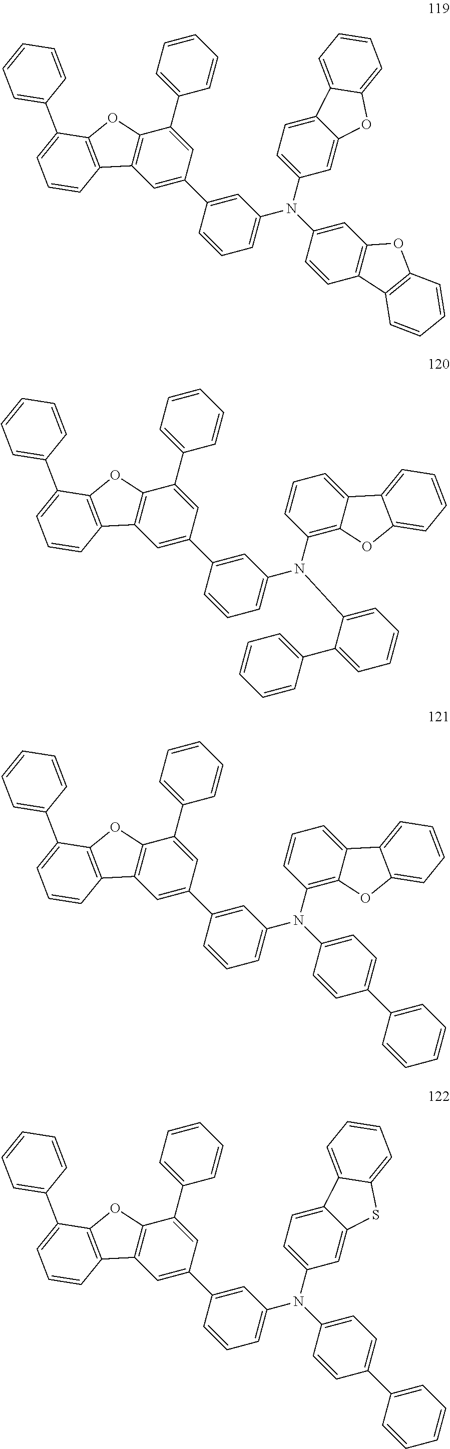 [SMILES: c1ccc(-c2cccc3c2oc2c(-c4ccccc4)cc(-c4cccc(N(c5ccc6c(c5)oc5ccccc56)c5ccc6c(c5)oc5ccccc56)c4)cc23)cc1 ; c1ccc(-c2ccccc2N(c2cccc(-c3cc(-c4ccccc4)c4oc5c(-c6ccccc6)cccc5c4c3)c2)c2cccc3c2oc2ccccc23)cc1 ; c1ccc(-c2ccc(N(c3cccc(-c4cc(-c5ccccc5)c5oc6c(-c7ccccc7)cccc6c5c4)c3)c3cccc4c3oc3ccccc34)cc2)cc1 ; c1ccc(-c2ccc(N(c3cccc(-c4cc(-c5ccccc5)c5oc6c(-c7ccccc7)cccc6c5c4)c3)c3ccc4c(c3)sc3ccccc34)cc2)cc1]

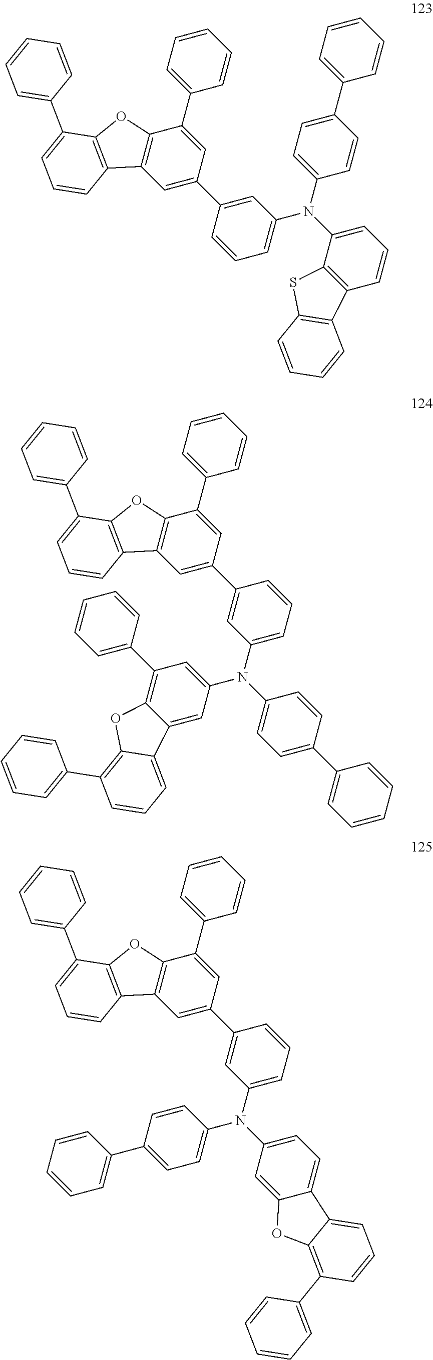 [SMILES: c1ccc(-c2ccc(N(c3cccc(-c4cc(-c5ccccc5)c5oc6c(-c7ccccc7)cccc6c5c4)c3)c3cccc4c3sc3ccccc34)cc2)cc1 ; c1ccc(-c2ccc(N(c3cccc(-c4cc(-c5ccccc5)c5oc6c(-c7ccccc7)cccc6c5c4)c3)c3cc(-c4ccccc4)c4oc5c(-c6ccccc6)cccc5c4c3)cc2)cc1 ; c1ccc(-c2ccc(N(c3cccc(-c4cc(-c5ccccc5)c5oc6c(-c7ccccc7)cccc6c5c4)c3)c3ccc4c(c3)oc3c(-c5ccccc5)cccc34)cc2)cc1]

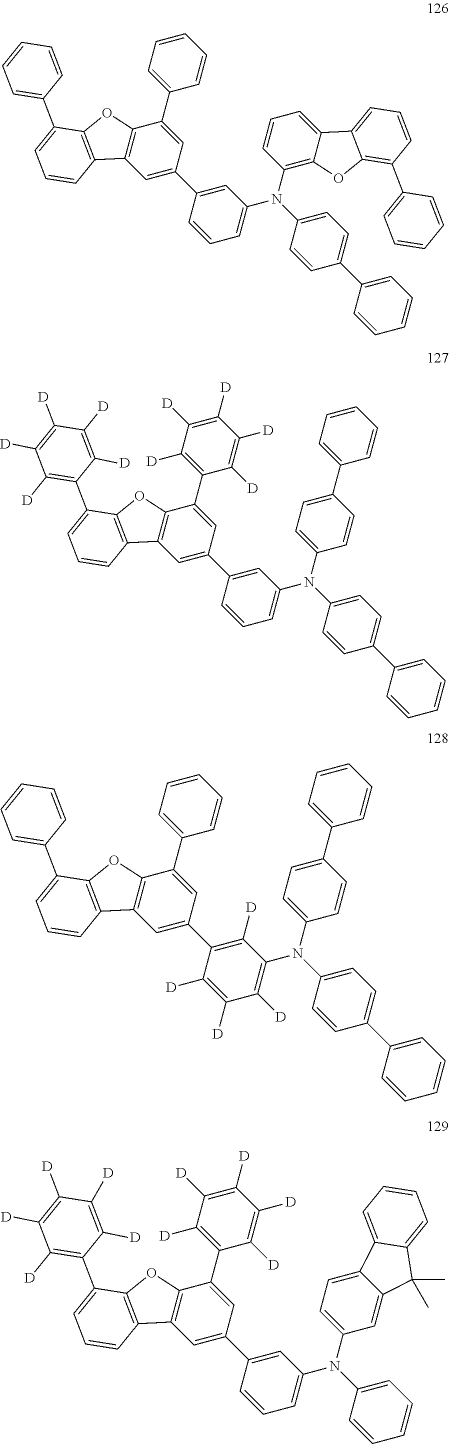

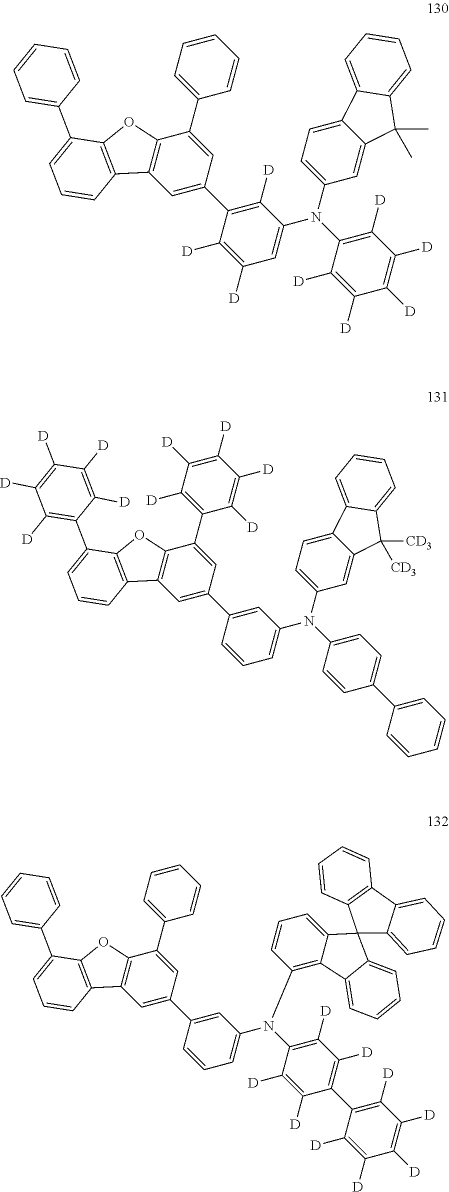 [SMILES: [2H]c1cc(N(c2ccc3c(c2)C(C)(C)c2ccccc2-3)c2c([2H])c([2H])c([2H])c([2H])c2[2H])c([2H])c(-c2cc(-c3ccccc3)c3oc4c(-c5ccccc5)cccc4c3c2)c1[2H] ; [2H]c1c([2H])c([2H])c(-c2cccc3c2oc2c(-c4c([2H])c([2H])c([2H])c([2H])c4[2H])cc(-c4cccc(N(c5ccc(-c6ccccc6)cc5)c5ccc6c(c5)C(C([2H])([2H])[2H])(C([2H])([2H])[2H])c5ccccc5-6)c4)cc23)c([2H])c1[2H] ; [2H]c1c([2H])c([2H])c(-c2c([2H])c([2H])c(N(c3cccc(-c4cc(-c5ccccc5)c5oc6c(-c7ccccc7)cccc6c5c4)c3)c3cccc4c3-c3ccccc3C43c4ccccc4-c4ccccc43)c([2H])c2[2H])c([2H])c1[2H]]

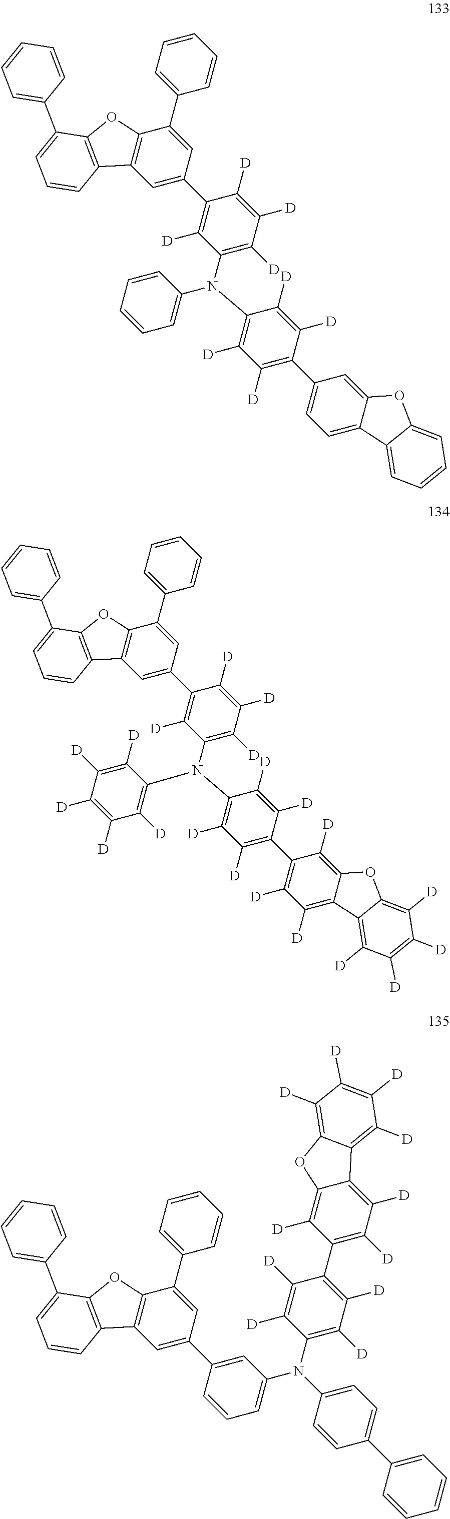

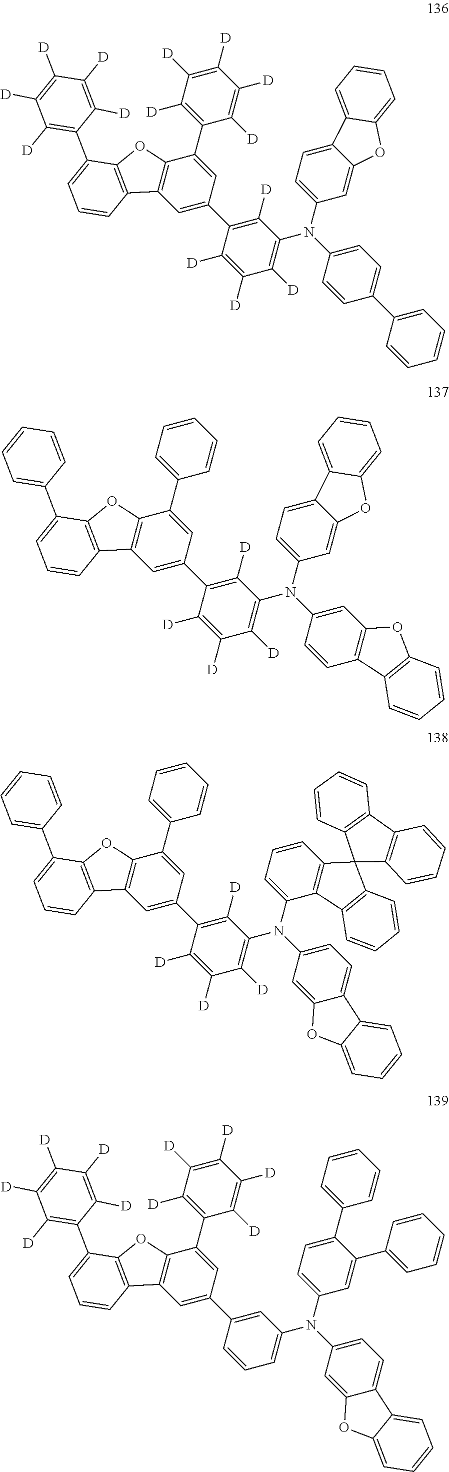

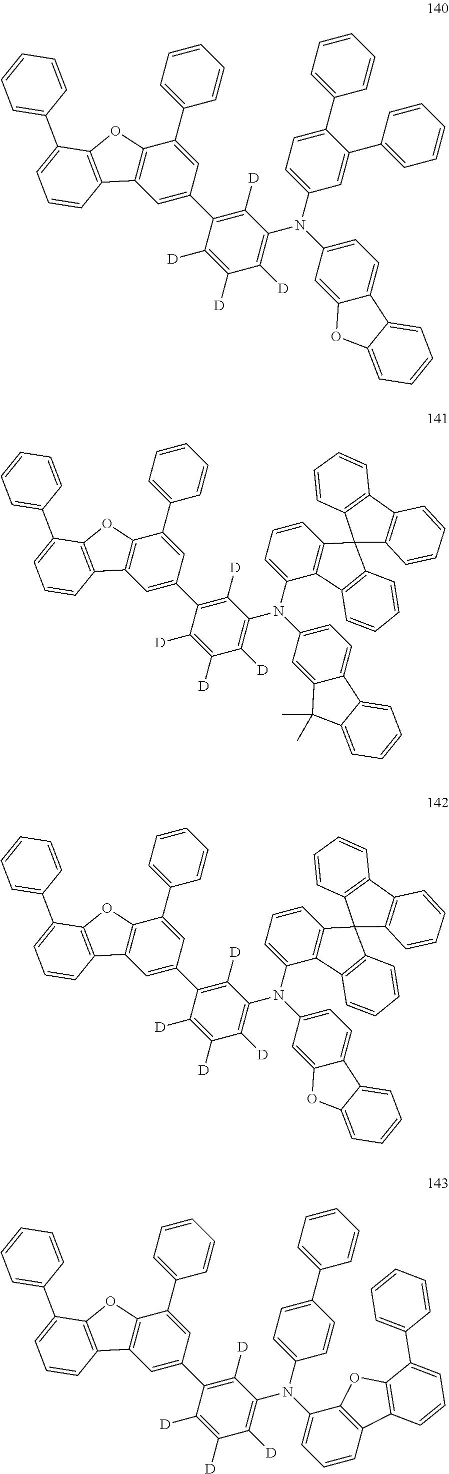

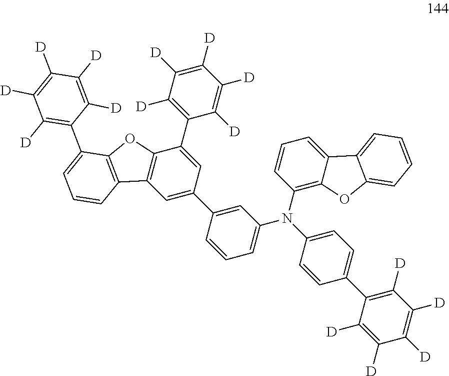 [SMILES: [2H]c1c([2H])c([2H])c(-c2ccc(N(c3cccc(-c4cc(-c5c([2H])c([2H])c([2H])c([2H])c5[2H])c5oc6c(-c7c([2H])c([2H])c([2H])c([2H])c7[2H])cccc6c5c4)c3)c3cccc4c3oc3ccccc34)cc2)c([2H])c1[2H]]

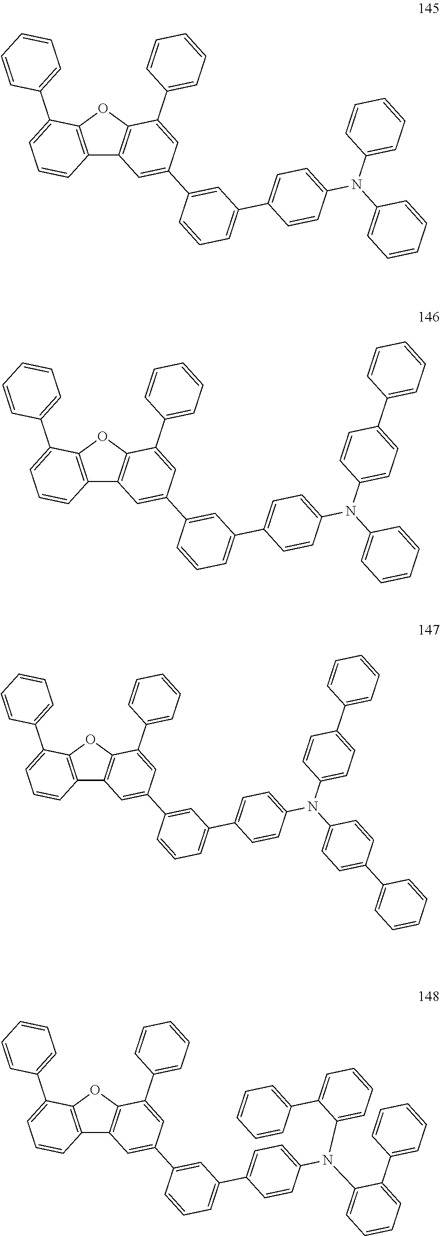 [SMILES: c1ccc(-c2cccc3c2oc2c(-c4ccccc4)cc(-c4cccc(-c5ccc(N(c6ccccc6)c6ccccc6)cc5)c4)cc23)cc1 ; c1ccc(-c2ccc(N(c3ccccc3)c3ccc(-c4cccc(-c5cc(-c6ccccc6)c6oc7c(-c8ccccc8)cccc7c6c5)c4)cc3)cc2)cc1 ; c1ccc(-c2ccc(N(c3ccc(-c4ccccc4)cc3)c3ccc(-c4cccc(-c5cc(-c6ccccc6)c6oc7c(-c8ccccc8)cccc7c6c5)c4)cc3)cc2)cc1 ; c1ccc(-c2ccccc2N(c2ccc(-c3cccc(-c4cc(-c5ccccc5)c5oc6c(-c7ccccc7)cccc6c5c4)c3)cc2)c2ccccc2-c2ccccc2)cc1]

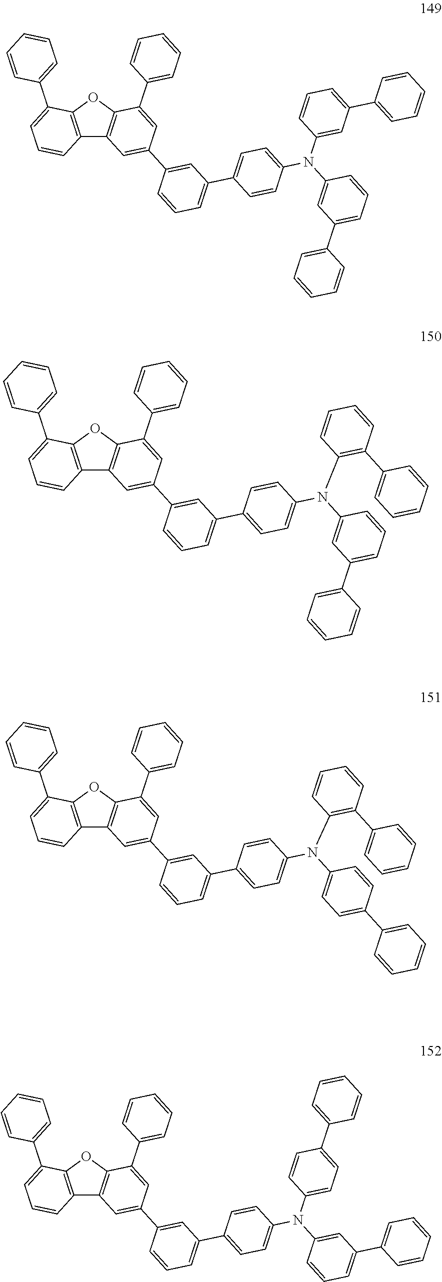 [SMILES: c1ccc(-c2cccc(N(c3ccc(-c4cccc(-c5cc(-c6ccccc6)c6oc7c(-c8ccccc8)cccc7c6c5)c4)cc3)c3cccc(-c4ccccc4)c3)c2)cc1 ; c1ccc(-c2cccc(N(c3ccc(-c4cccc(-c5cc(-c6ccccc6)c6oc7c(-c8ccccc8)cccc7c6c5)c4)cc3)c3ccccc3-c3ccccc3)c2)cc1 ; c1ccc(-c2ccc(N(c3ccc(-c4cccc(-c5cc(-c6ccccc6)c6oc7c(-c8ccccc8)cccc7c6c5)c4)cc3)c3ccccc3-c3ccccc3)cc2)cc1 ; c1ccc(-c2ccc(N(c3ccc(-c4cccc(-c5cc(-c6ccccc6)c6oc7c(-c8ccccc8)cccc7c6c5)c4)cc3)c3cccc(-c4ccccc4)c3)cc2)cc1]

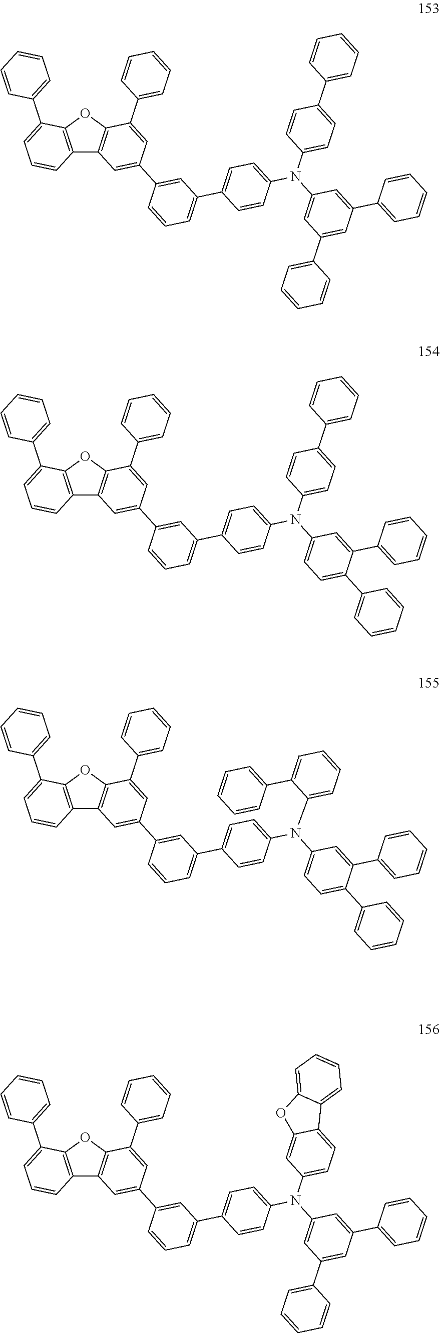 [SMILES: c1ccc(-c2ccc(N(c3ccc(-c4cccc(-c5cc(-c6ccccc6)c6oc7c(-c8ccccc8)cccc7c6c5)c4)cc3)c3cc(-c4ccccc4)cc(-c4ccccc4)c3)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc(-c4ccccc4)c(-c4ccccc4)c3)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccccc3-c3ccccc3)cc2-c2ccccc2)cc1 ; c1ccc(-c2cc(-c3ccccc3)cc(N(c3ccc(-c4cccc(-c5cc(-c6ccccc6)c6oc7c(-c8ccccc8)cccc7c6c5)c4)cc3)c3ccc4c(c3)oc3ccccc34)c2)cc1]

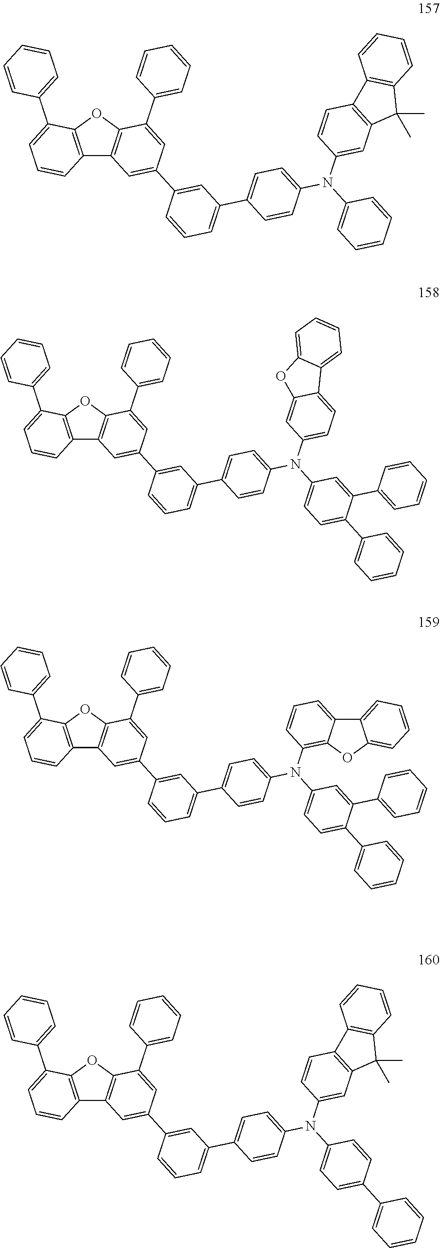 [SMILES: CC1(C)c2ccccc2-c2ccc(N(c3ccccc3)c3ccc(-c4cccc(-c5cc(-c6ccccc6)c6oc7c(-c8ccccc8)cccc7c6c5)c4)cc3)cc21 ; c1ccc(-c2ccc(N(c3ccc(-c4cccc(-c5cc(-c6ccccc6)c6oc7c(-c8ccccc8)cccc7c6c5)c4)cc3)c3ccc4c(c3)oc3ccccc34)cc2-c2ccccc2)cc1 ; c1ccc(-c2ccc(N(c3ccc(-c4cccc(-c5cc(-c6ccccc6)c6oc7c(-c8ccccc8)cccc7c6c5)c4)cc3)c3cccc4c3oc3ccccc34)cc2-c2ccccc2)cc1 ; CC1(C)c2ccccc2-c2ccc(N(c3ccc(-c4ccccc4)cc3)c3ccc(-c4cccc(-c5cc(-c6ccccc6)c6oc7c(-c8ccccc8)cccc7c6c5)c4)cc3)cc21]

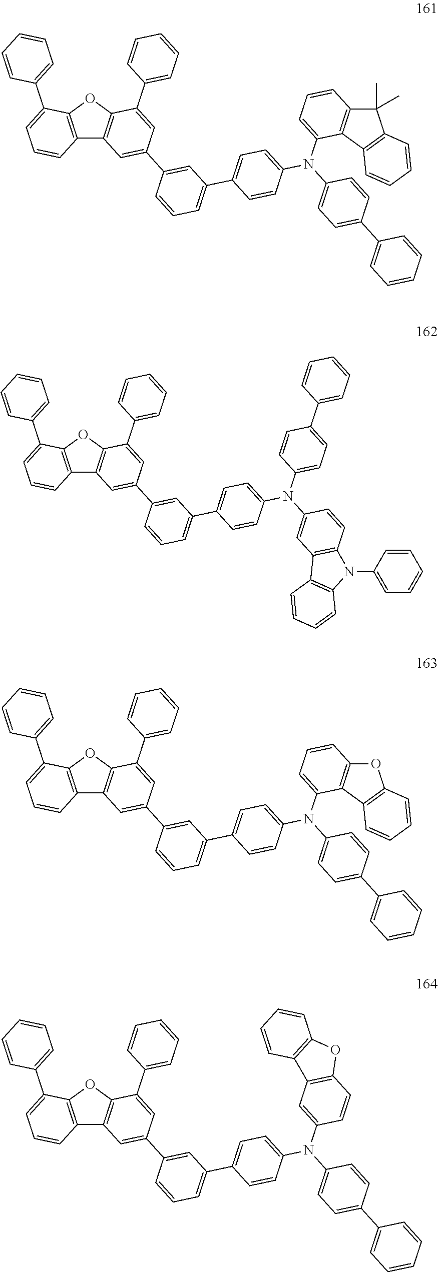 [SMILES: CC1(C)c2ccccc2-c2c(N(c3ccc(-c4ccccc4)cc3)c3ccc(-c4cccc(-c5cc(-c6ccccc6)c6oc7c(-c8ccccc8)cccc7c6c5)c4)cc3)cccc21 ; c1ccc(-c2ccc(N(c3ccc(-c4cccc(-c5cc(-c6ccccc6)c6oc7c(-c8ccccc8)cccc7c6c5)c4)cc3)c3ccc4c(c3)c3ccccc3n4-c3ccccc3)cc2)cc1 ; c1ccc(-c2ccc(N(c3ccc(-c4cccc(-c5cc(-c6ccccc6)c6oc7c(-c8ccccc8)cccc7c6c5)c4)cc3)c3cccc4oc5ccccc5c34)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4oc5ccccc5c4c3)cc2)cc1]

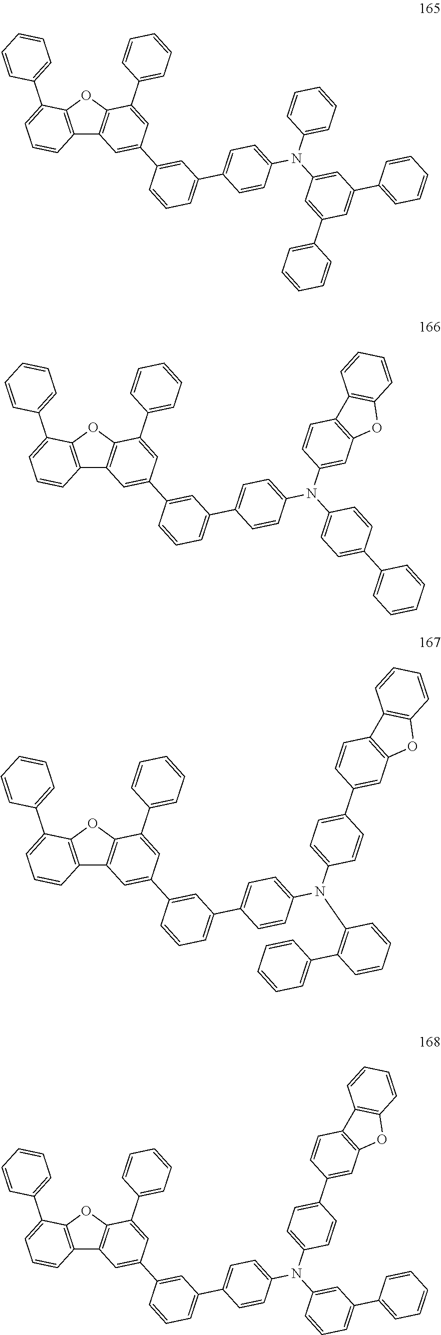 [SMILES: c1ccc(-c2cc(-c3ccccc3)cc(N(c3ccccc3)c3ccc(-c4cccc(-c5cc(-c6ccccc6)c6oc7c(-c8ccccc8)cccc7c6c5)c4)cc3)c2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)oc3ccccc34)cc2)cc1 ; c1ccc(-c2ccccc2N(c2ccc(-c3cccc(-c4cc(-c5ccccc5)c5oc6c(-c7ccccc7)cccc6c5c4)c3)cc2)c2ccc(-c3ccc4c(c3)oc3ccccc34)cc2)cc1 ; c1ccc(-c2cccc(N(c3ccc(-c4cccc(-c5cc(-c6ccccc6)c6oc7c(-c8ccccc8)cccc7c6c5)c4)cc3)c3ccc(-c4ccc5c(c4)oc4ccccc45)cc3)c2)cc1]

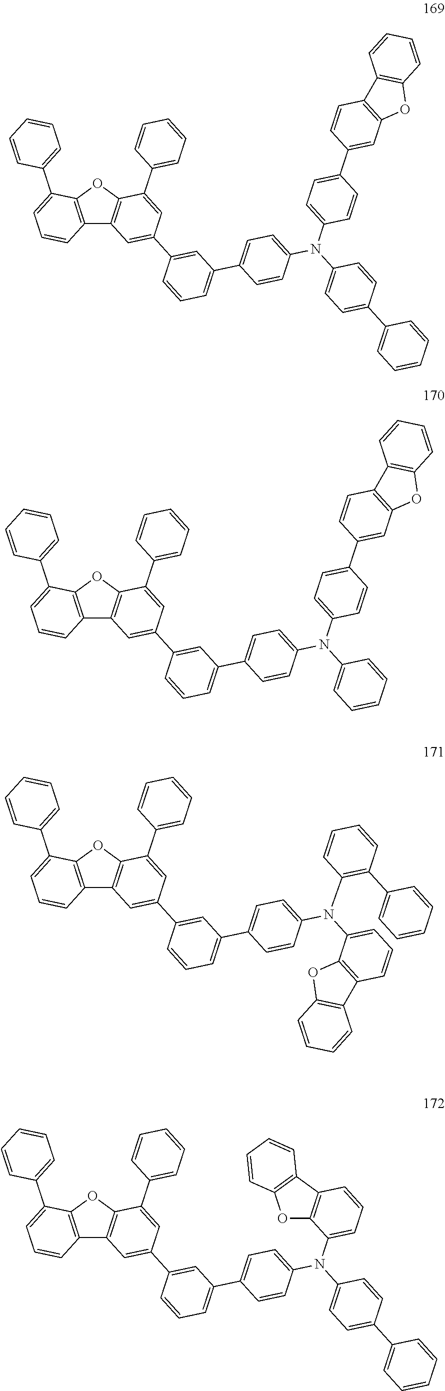 [SMILES: c1ccc(-c2ccc(N(c3ccc(-c4cccc(-c5cc(-c6ccccc6)c6oc7c(-c8ccccc8)cccc7c6c5)c4)cc3)c3ccc(-c4ccc5c(c4)oc4ccccc45)cc3)cc2)cc1 ; c1ccc(-c2cccc3c2oc2c(-c4ccccc4)cc(-c4cccc(-c5ccc(N(c6ccccc6)c6ccc(-c7ccc8c(c7)oc7ccccc78)cc6)cc5)c4)cc23)cc1 ; c1ccc(-c2ccccc2N(c2ccc(-c3cccc(-c4cc(-c5ccccc5)c5oc6c(-c7ccccc7)cccc6c5c4)c3)cc2)c2cccc3c2oc2ccccc23)cc1 ; c1ccc(-c2ccc(N(c3ccc(-c4cccc(-c5cc(-c6ccccc6)c6oc7c(-c8ccccc8)cccc7c6c5)c4)cc3)c3cccc4c3oc3ccccc34)cc2)cc1]

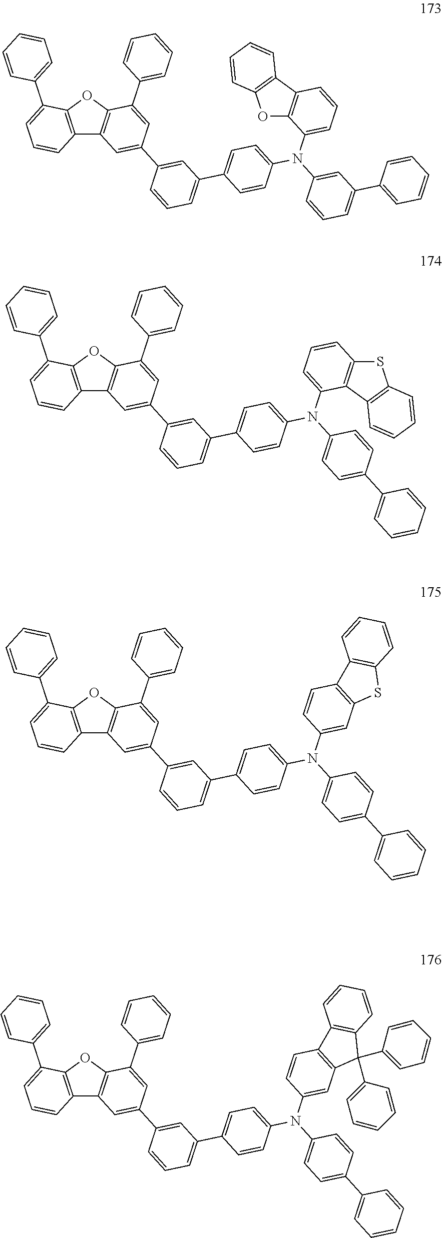 [SMILES: c1ccc(-c2cccc(N(c3ccc(-c4cccc(-c5cc(-c6ccccc6)c6oc7c(-c8ccccc8)cccc7c6c5)c4)cc3)c3cccc4c3oc3ccccc34)c2)cc1 ; c1ccc(-c2ccc(N(c3ccc(-c4cccc(-c5cc(-c6ccccc6)c6oc7c(-c8ccccc8)cccc7c6c5)c4)cc3)c3cccc4sc5ccccc5c34)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)sc3ccccc34)cc2)cc1 ; c1ccc(-c2ccc(N(c3ccc(-c4cccc(-c5cc(-c6ccccc6)c6oc7c(-c8ccccc8)cccc7c6c5)c4)cc3)c3ccc4c(c3)C(c3ccccc3)(c3ccccc3)c3ccccc3-4)cc2)cc1]

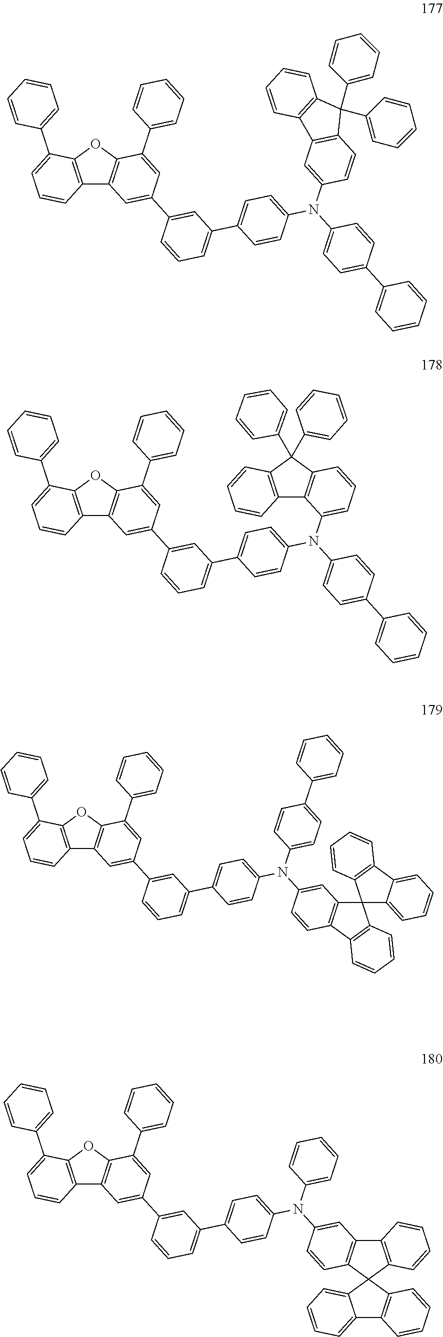

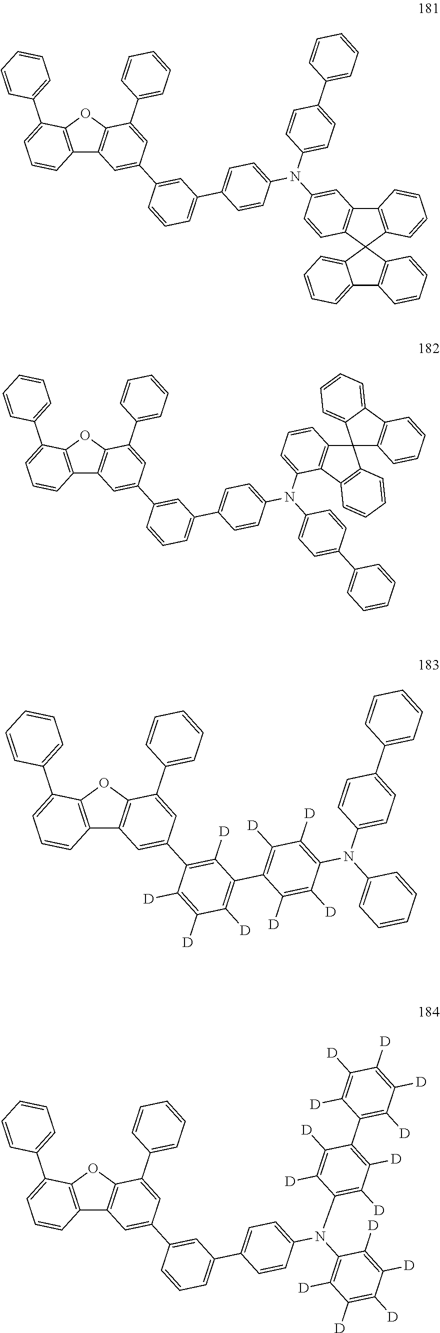

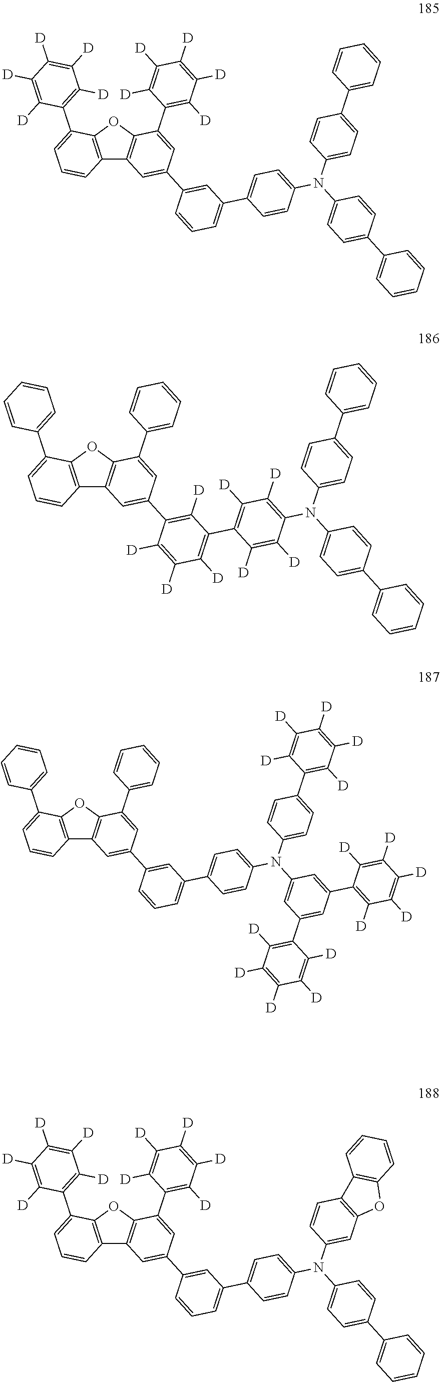

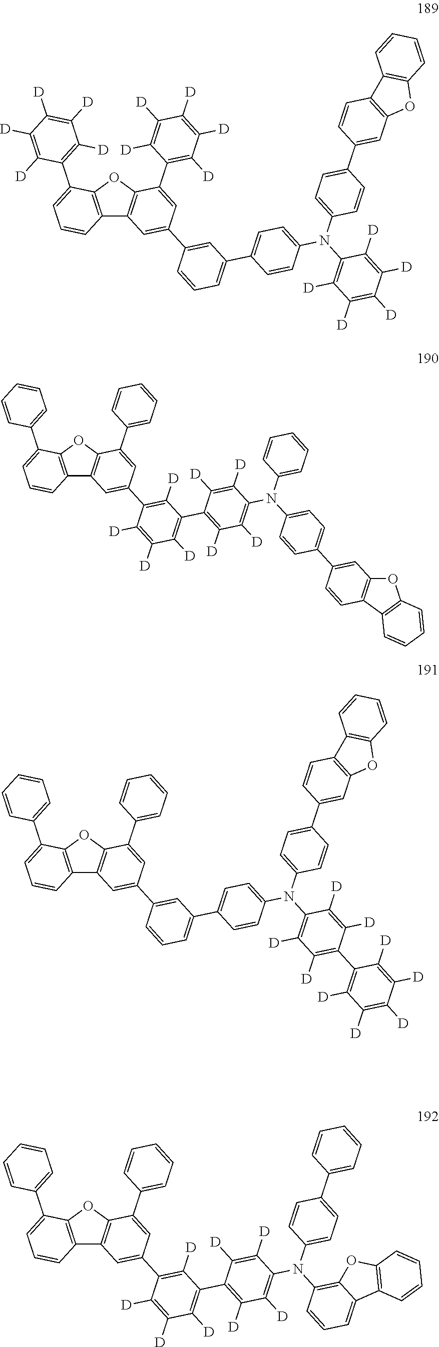

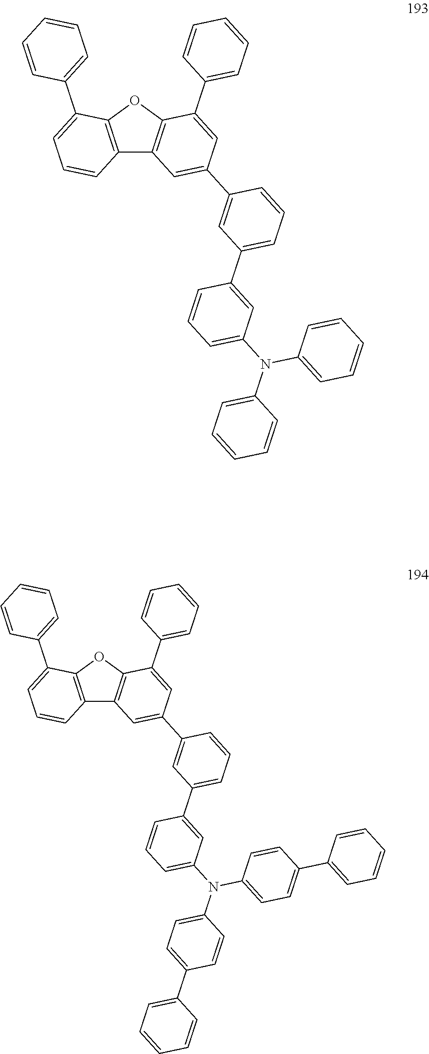 [SMILES: c1ccc(-c2cccc3c2oc2c(-c4ccccc4)cc(-c4cccc(-c5cccc(N(c6ccccc6)c6ccccc6)c5)c4)cc23)cc1 ; c1ccc(-c2ccc(N(c3ccc(-c4ccccc4)cc3)c3cccc(-c4cccc(-c5cc(-c6ccccc6)c6oc7c(-c8ccccc8)cccc7c6c5)c4)c3)cc2)cc1]

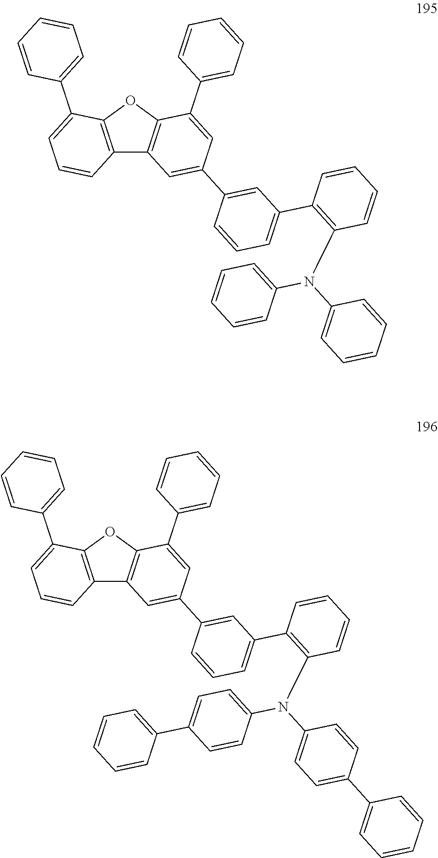 [SMILES: c1ccc(-c2cccc3c2oc2c(-c4ccccc4)cc(-c4cccc(-c5ccccc5N(c5ccccc5)c5ccccc5)c4)cc23)cc1 ; c1ccc(-c2ccc(N(c3ccc(-c4ccccc4)cc3)c3ccccc3-c3cccc(-c4cc(-c5ccccc5)c5oc6c(-c7ccccc7)cccc6c5c4)c3)cc2)cc1]

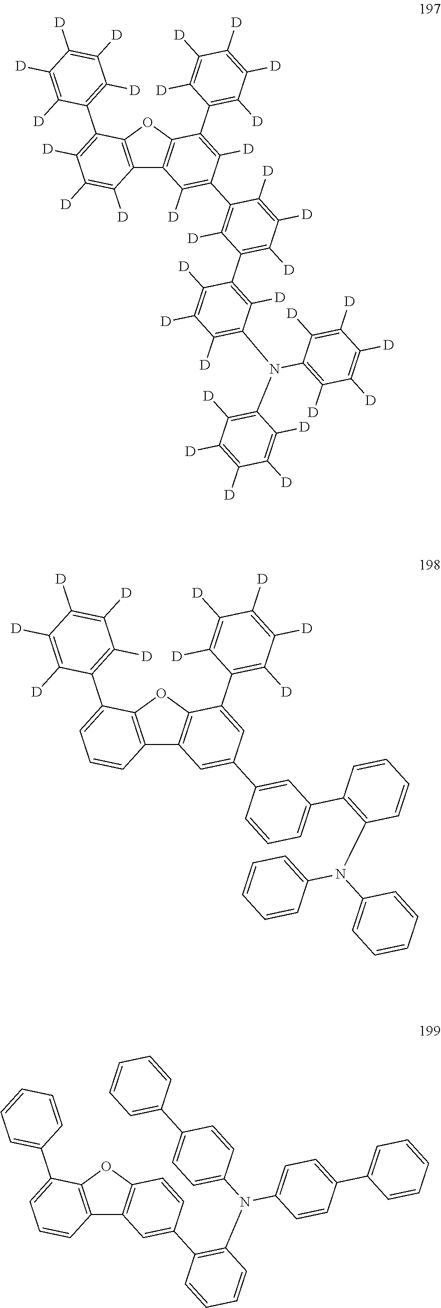 [SMILES: [2H]c1c([2H])c([2H])c(-c2c([2H])c([2H])c([2H])c3c2oc2c(-c4c([2H])c([2H])c([2H])c([2H])c4[2H])c([2H])c(-c4c([2H])c([2H])c([2H])c(-c5c([2H])c([2H])c([2H])c(N(c6c([2H])c([2H])c([2H])c([2H])c6[2H])c6c([2H])c([2H])c([2H])c([2H])c6[2H])c5[2H])c4[2H])c([2H])c23)c([2H])c1[2H] ; [2H]c1c([2H])c([2H])c(-c2cccc3c2oc2c(-c4c([2H])c([2H])c([2H])c([2H])c4[2H])cc(-c4cccc(-c5ccccc5N(c5ccccc5)c5ccccc5)c4)cc23)c([2H])c1[2H] ; c1ccc(-c2ccc(N(c3ccc(-c4ccccc4)cc3)c3ccccc3-c3ccc4oc5c(-c6ccccc6)cccc5c4c3)cc2)cc1]

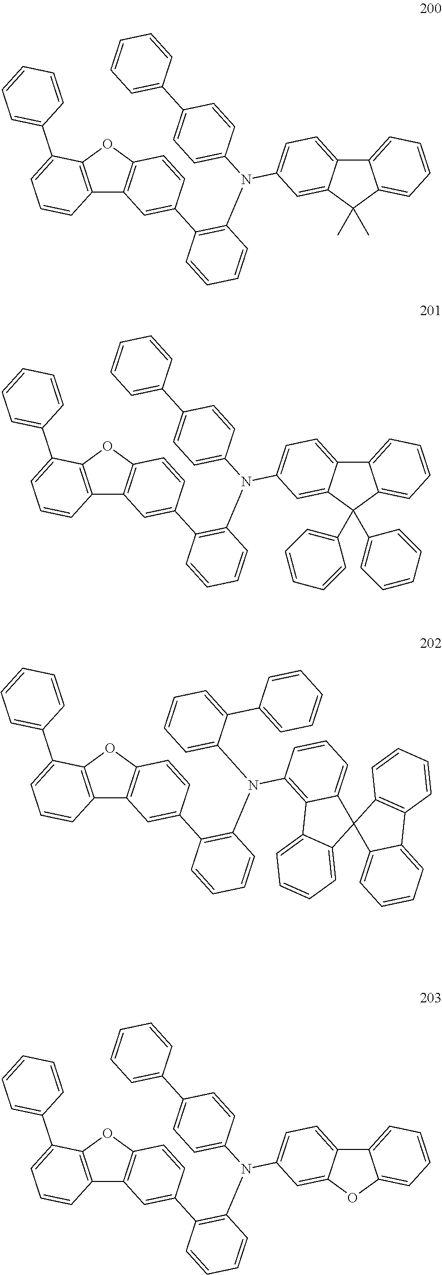 [SMILES: CC1(C)c2ccccc2-c2ccc(N(c3ccc(-c4ccccc4)cc3)c3ccccc3-c3ccc4oc5c(-c6ccccc6)cccc5c4c3)cc21 ; c1ccc(-c2ccc(N(c3ccc4c(c3)C(c3ccccc3)(c3ccccc3)c3ccccc3-4)c3ccccc3-c3ccc4oc5c(-c6ccccc6)cccc5c4c3)cc2)cc1 ; c1ccc(-c2ccccc2N(c2ccccc2-c2ccc3oc4c(-c5ccccc5)cccc4c3c2)c2cccc3c2-c2ccccc2C32c3ccccc3-c3ccccc32)cc1 ; c1ccc(-c2ccc(N(c3ccc4c(c3)oc3ccccc34)c3ccccc3-c3ccc4oc5c(-c6ccccc6)cccc5c4c3)cc2)cc1]

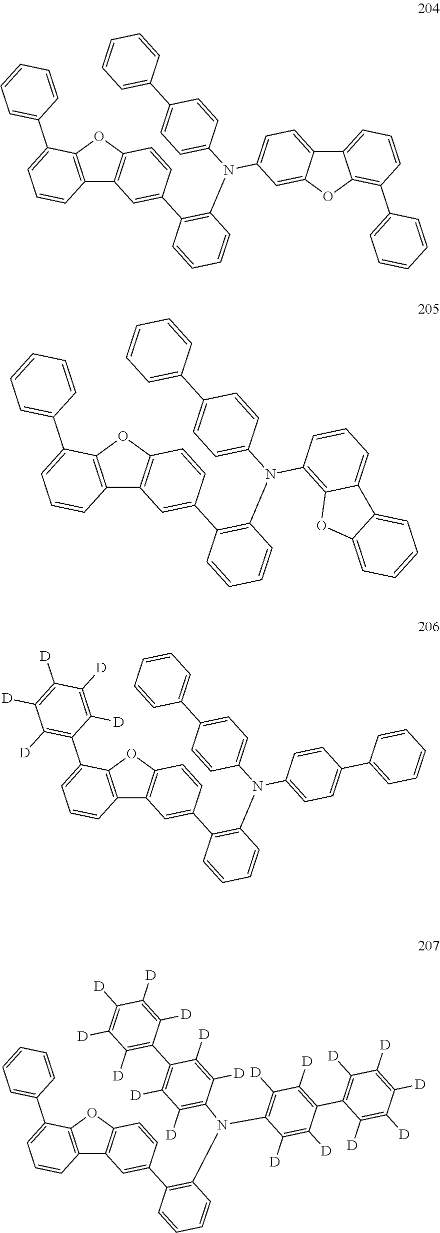 [SMILES: c1ccc(-c2ccc(N(c3ccc4c(c3)oc3c(-c5ccccc5)cccc34)c3ccccc3-c3ccc4oc5c(-c6ccccc6)cccc5c4c3)cc2)cc1 ; c1ccc(-c2ccc(N(c3ccccc3-c3ccc4oc5c(-c6ccccc6)cccc5c4c3)c3cccc4c3oc3ccccc34)cc2)cc1 ; [2H]c1c([2H])c([2H])c(-c2cccc3c2oc2ccc(-c4ccccc4N(c4ccc(-c5ccccc5)cc4)c4ccc(-c5ccccc5)cc4)cc23)c([2H])c1[2H] ; [2H]c1c([2H])c([2H])c(-c2c([2H])c([2H])c(N(c3ccccc3-c3ccc4oc5c(-c6ccccc6)cccc5c4c3)c3c([2H])c([2H])c(-c4c([2H])c([2H])c([2H])c([2H])c4[2H])c([2H])c3[2H])c([2H])c2[2H])c([2H])c1[2H]]

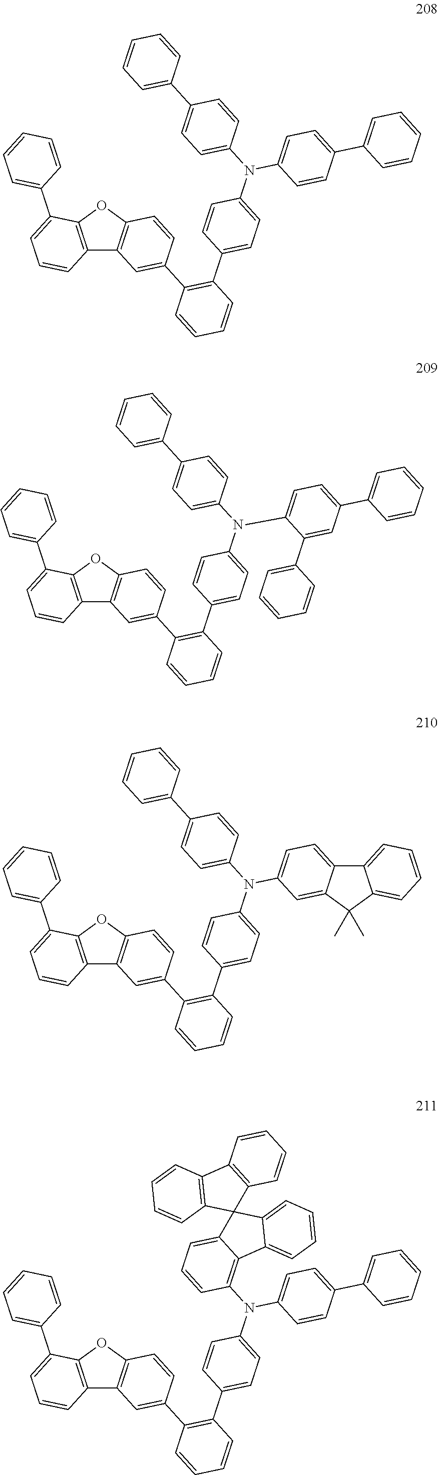 [SMILES: c1ccc(-c2ccc(N(c3ccc(-c4ccccc4)cc3)c3ccc(-c4ccccc4-c4ccc5oc6c(-c7ccccc7)cccc6c5c4)cc3)cc2)cc1 ; c1ccc(-c2ccc(N(c3ccc(-c4ccccc4-c4ccc5oc6c(-c7ccccc7)cccc6c5c4)cc3)c3ccc(-c4ccccc4)cc3-c3ccccc3)cc2)cc1 ; CC1(C)c2ccccc2-c2ccc(N(c3ccc(-c4ccccc4)cc3)c3ccc(-c4ccccc4-c4ccc5oc6c(-c7ccccc7)cccc6c5c4)cc3)cc21 ; c1ccc(-c2ccc(N(c3ccc(-c4ccccc4-c4ccc5oc6c(-c7ccccc7)cccc6c5c4)cc3)c3cccc4c3-c3ccccc3C43c4ccccc4-c4ccccc43)cc2)cc1]

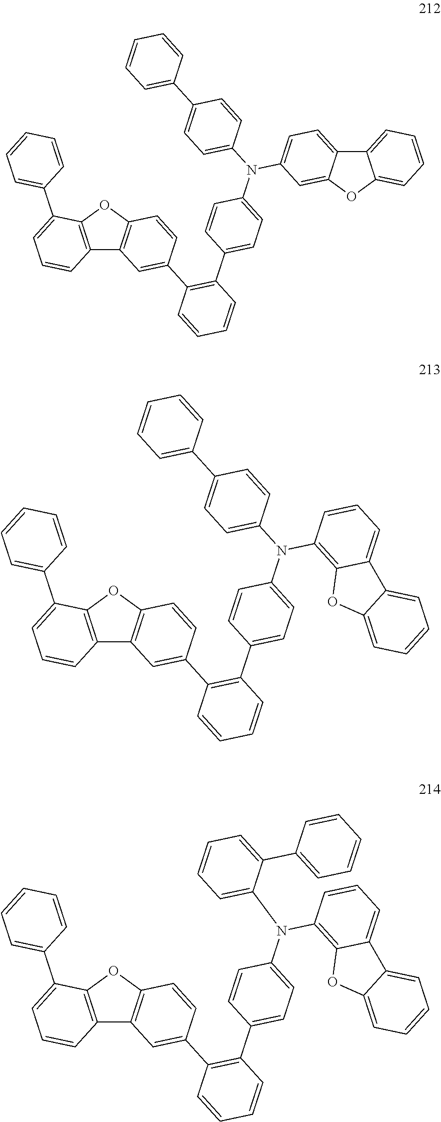 [SMILES: c1ccc(-c2ccc(N(c3ccc(-c4ccccc4-c4ccc5oc6c(-c7ccccc7)cccc6c5c4)cc3)c3ccc4c(c3)oc3ccccc34)cc2)cc1 ; c1ccc(-c2ccc(N(c3ccc(-c4ccccc4-c4ccc5oc6c(-c7ccccc7)cccc6c5c4)cc3)c3cccc4c3oc3ccccc34)cc2)cc1 ; c1ccc(-c2ccccc2N(c2ccc(-c3ccccc3-c3ccc4oc5c(-c6ccccc6)cccc5c4c3)cc2)c2cccc3c2oc2ccccc23)cc1]

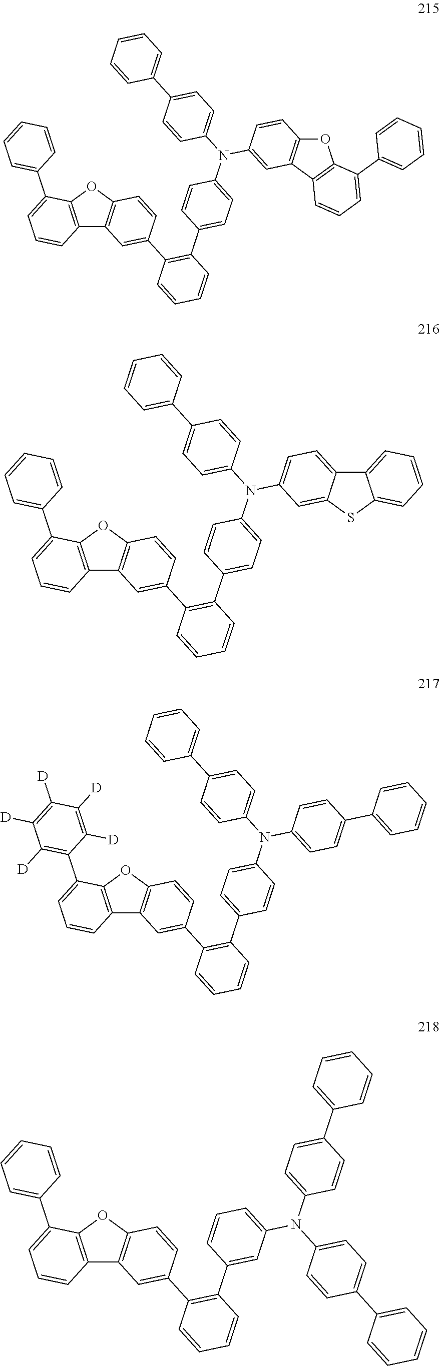 [SMILES: c1ccc(-c2ccc(N(c3ccc(-c4ccccc4-c4ccc5oc6c(-c7ccccc7)cccc6c5c4)cc3)c3ccc4oc5c(-c6ccccc6)cccc5c4c3)cc2)cc1 ; c1ccc(-c2ccc(N(c3ccc(-c4ccccc4-c4ccc5oc6c(-c7ccccc7)cccc6c5c4)cc3)c3ccc4c(c3)sc3ccccc34)cc2)cc1 ; [2H]c1c([2H])c([2H])c(-c2cccc3c2oc2ccc(-c4ccccc4-c4ccc(N(c5ccc(-c6ccccc6)cc5)c5ccc(-c6ccccc6)cc5)cc4)cc23)c([2H])c1[2H] ; c1ccc(-c2ccc(N(c3ccc(-c4ccccc4)cc3)c3cccc(-c4ccccc4-c4ccc5oc6c(-c7ccccc7)cccc6c5c4)c3)cc2)cc1]

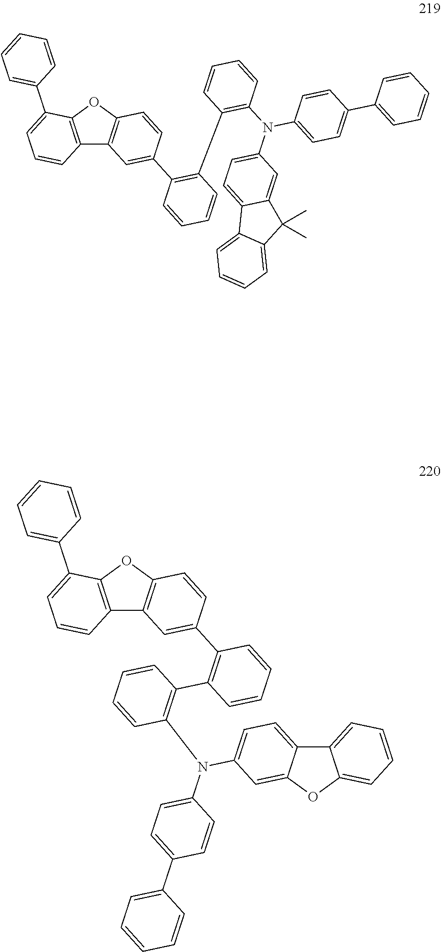 [SMILES: CC1(C)c2ccccc2-c2ccc(N(c3ccc(-c4ccccc4)cc3)c3ccccc3-c3ccccc3-c3ccc4oc5c(-c6ccccc6)cccc5c4c3)cc21 ; c1ccc(-c2ccc(N(c3ccc4c(c3)oc3ccccc34)c3ccccc3-c3ccccc3-c3ccc4oc5c(-c6ccccc6)cccc5c4c3)cc2)cc1]

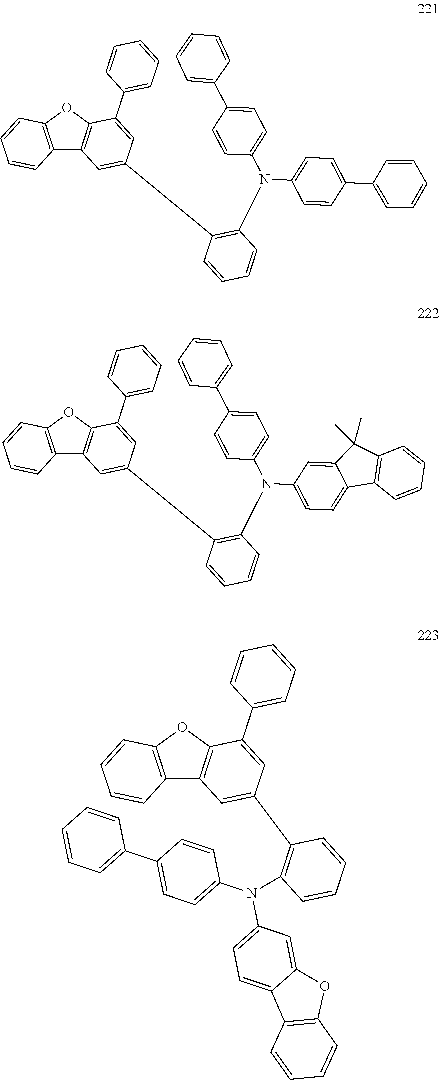 [SMILES: c1ccc(-c2ccc(N(c3ccc(-c4ccccc4)cc3)c3ccccc3-c3cc(-c4ccccc4)c4oc5ccccc5c4c3)cc2)cc1 ; CC1(C)c2ccccc2-c2ccc(N(c3ccc(-c4ccccc4)cc3)c3ccccc3-c3cc(-c4ccccc4)c4oc5ccccc5c4c3)cc21 ; c1ccc(-c2ccc(N(c3ccc4c(c3)oc3ccccc34)c3ccccc3-c3cc(-c4ccccc4)c4oc5ccccc5c4c3)cc2)cc1]

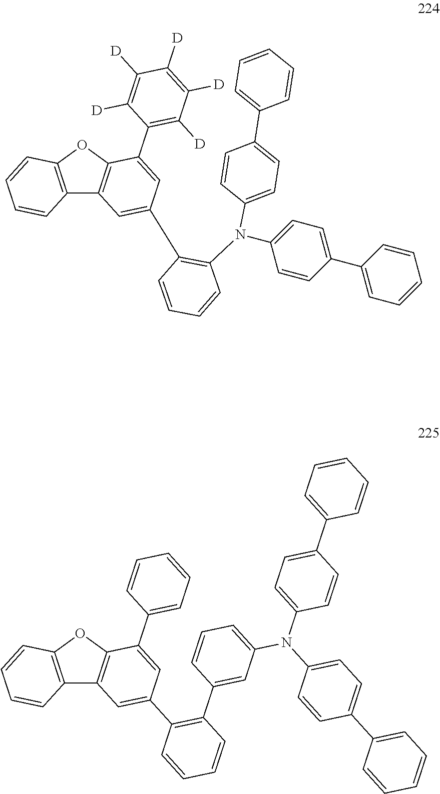 [SMILES: [2H]c1c([2H])c([2H])c(-c2cc(-c3ccccc3N(c3ccc(-c4ccccc4)cc3)c3ccc(-c4ccccc4)cc3)cc3c2oc2ccccc23)c([2H])c1[2H] ; c1ccc(-c2ccc(N(c3ccc(-c4ccccc4)cc3)c3cccc(-c4ccccc4-c4cc(-c5ccccc5)c5oc6ccccc6c5c4)c3)cc2)cc1]

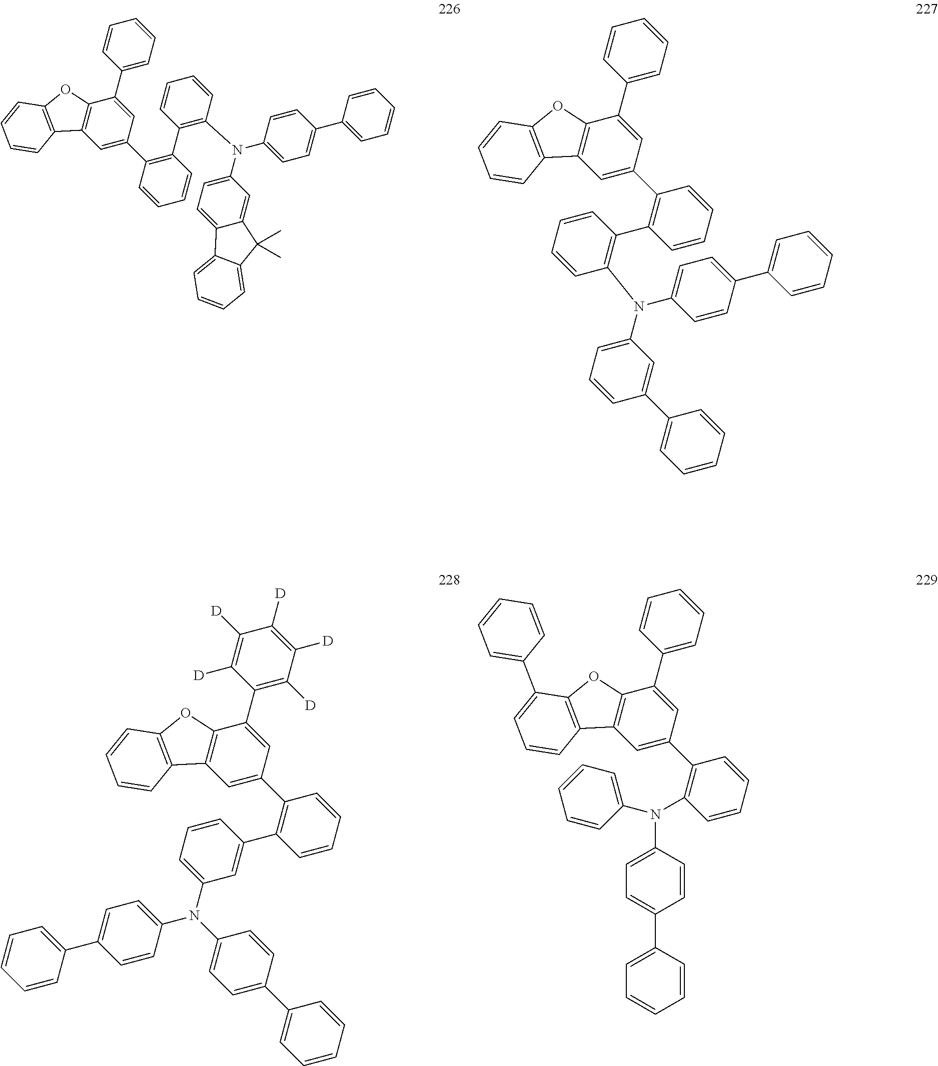 [SMILES: CC1(C)c2ccccc2-c2ccc(N(c3ccc(-c4ccccc4)cc3)c3ccccc3-c3ccccc3-c3cc(-c4ccccc4)c4oc5ccccc5c4c3)cc21 ; c1ccc(-c2ccc(N(c3cccc(-c4ccccc4)c3)c3ccccc3-c3ccccc3-c3cc(-c4ccccc4)c4oc5ccccc5c4c3)cc2)cc1 ; [2H]c1c([2H])c([2H])c(-c2cc(-c3ccccc3-c3cccc(N(c4ccc(-c5ccccc5)cc4)c4ccc(-c5ccccc5)cc4)c3)cc3c2oc2ccccc23)c([2H])c1[2H] ; c1ccc(-c2ccc(N(c3ccccc3)c3ccccc3-c3cc(-c4ccccc4)c4oc5c(-c6ccccc6)cccc5c4c3)cc2)cc1]

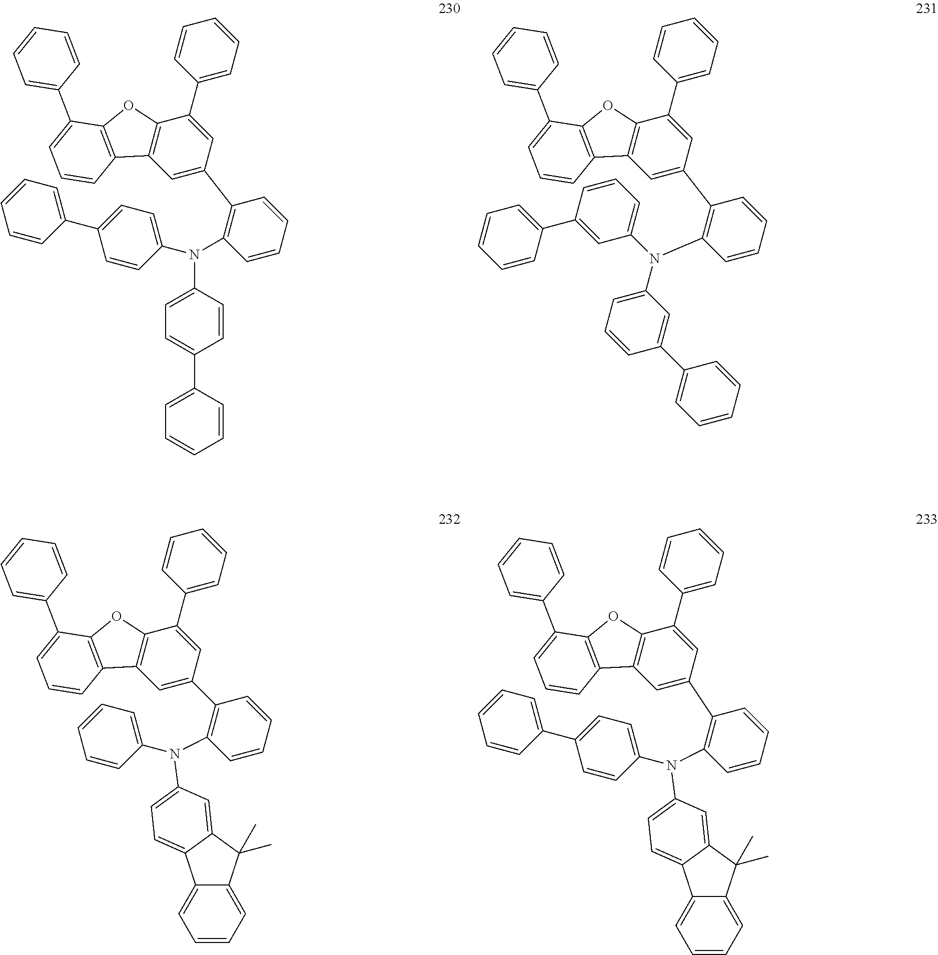 [SMILES: c1ccc(-c2ccc(N(c3ccc(-c4ccccc4)cc3)c3ccccc3-c3cc(-c4ccccc4)c4oc5c(-c6ccccc6)cccc5c4c3)cc2)cc1 ; c1ccc(-c2cccc(N(c3cccc(-c4ccccc4)c3)c3ccccc3-c3cc(-c4ccccc4)c4oc5c(-c6ccccc6)cccc5c4c3)c2)cc1 ; CC1(C)c2ccccc2-c2ccc(N(c3ccccc3)c3ccccc3-c3cc(-c4ccccc4)c4oc5c(-c6ccccc6)cccc5c4c3)cc21 ; CC1(C)c2ccccc2-c2ccc(N(c3ccc(-c4ccccc4)cc3)c3ccccc3-c3cc(-c4ccccc4)c4oc5c(-c6ccccc6)cccc5c4c3)cc21]

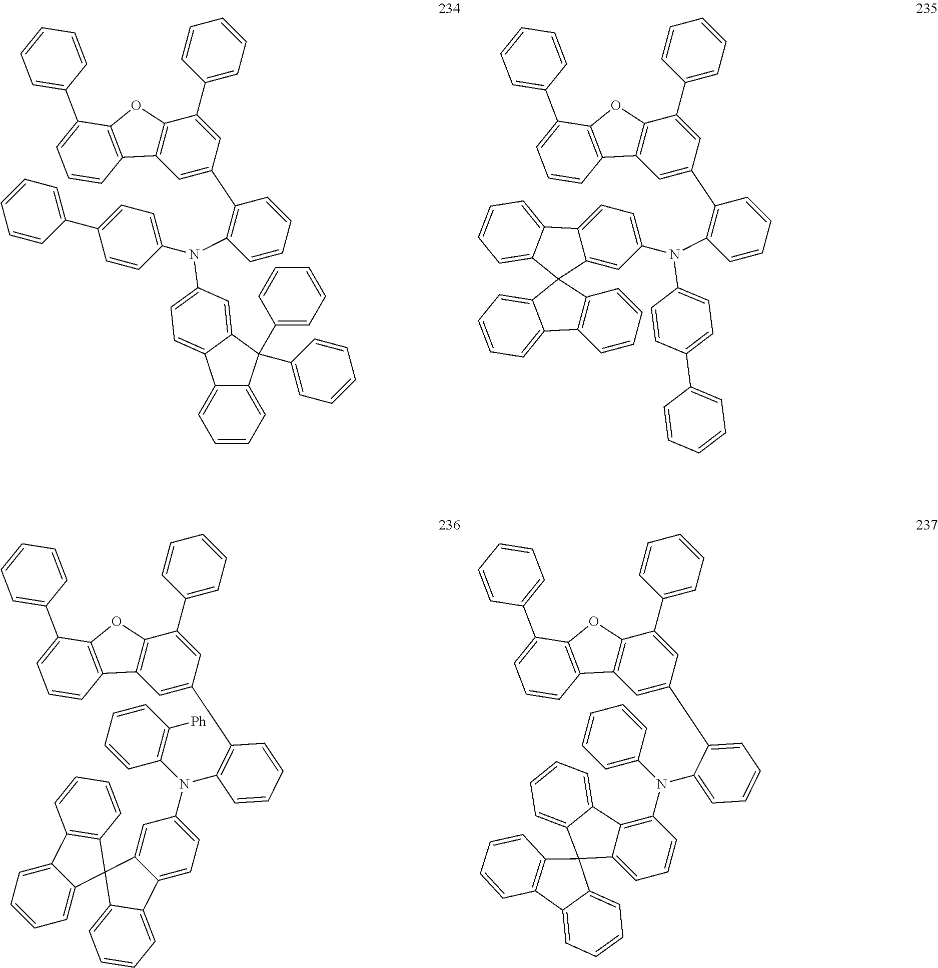 [SMILES: c1ccc(-c2ccc(N(c3ccc4c(c3)C(c3ccccc3)(c3ccccc3)c3ccccc3-4)c3ccccc3-c3cc(-c4ccccc4)c4oc5c(-c6ccccc6)cccc5c4c3)cc2)cc1 ; c1ccc(-c2ccc(N(c3ccc4c(c3)C3(c5ccccc5-c5ccccc53)c3ccccc3-4)c3ccccc3-c3cc(-c4ccccc4)c4oc5c(-c6ccccc6)cccc5c4c3)cc2)cc1 ; c1ccc(-c2ccccc2N(c2ccc3c(c2)C2(c4ccccc4-c4ccccc42)c2ccccc2-3)c2ccccc2-c2cc(-c3ccccc3)c3oc4c(-c5ccccc5)cccc4c3c2)cc1 ; c1ccc(-c2cccc3c2oc2c(-c4ccccc4)cc(-c4ccccc4N(c4ccccc4)c4cccc5c4-c4ccccc4C54c5ccccc5-c5ccccc54)cc23)cc1]

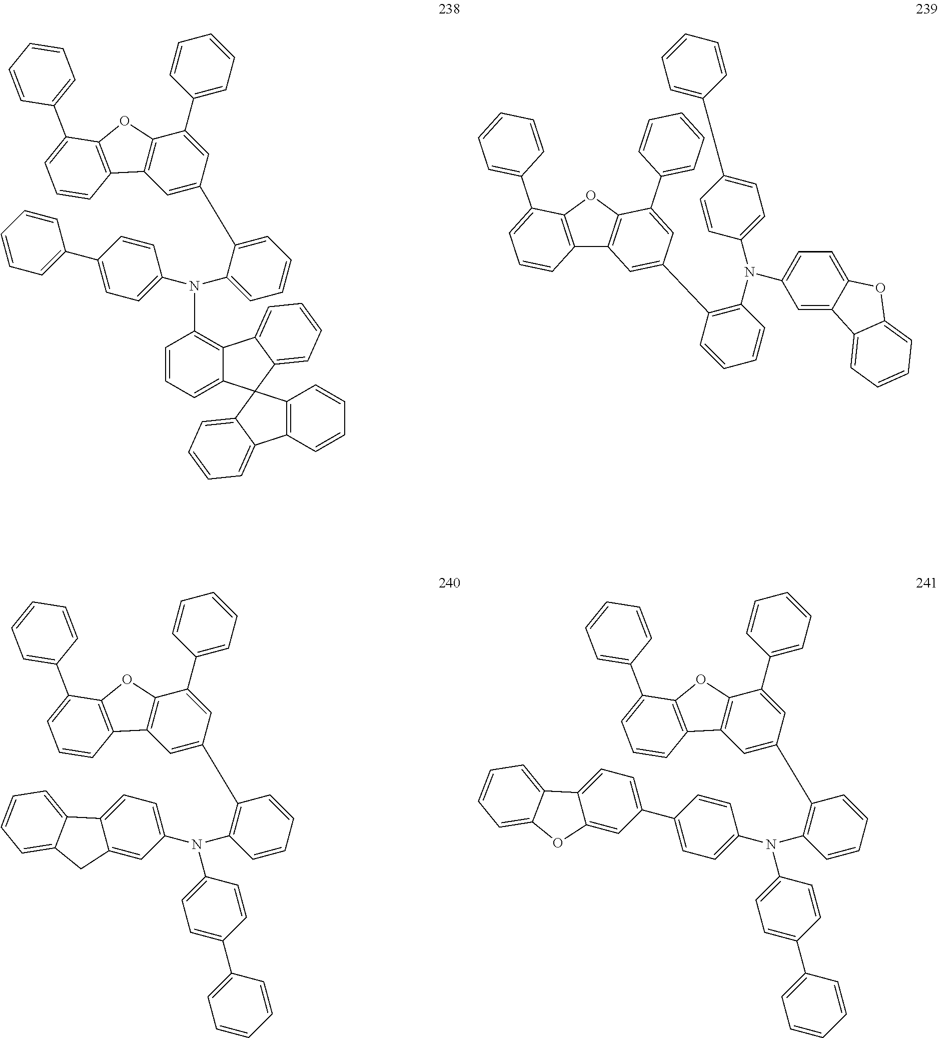 [SMILES: c1ccc(-c2ccc(N(c3ccccc3-c3cc(-c4ccccc4)c4oc5c(-c6ccccc6)cccc5c4c3)c3cccc4c3-c3ccccc3C43c4ccccc4-c4ccccc43)cc2)cc1 ; c1ccc(-c2ccc(N(c3ccc4oc5ccccc5c4c3)c3ccccc3-c3cc(-c4ccccc4)c4oc5c(-c6ccccc6)cccc5c4c3)cc2)cc1 ; c1ccc(-c2ccc(N(c3ccc4c(c3)Cc3ccccc3-4)c3ccccc3-c3cc(-c4ccccc4)c4oc5c(-c6ccccc6)cccc5c4c3)cc2)cc1 ; c1ccc(-c2ccc(N(c3ccc(-c4ccc5c(c4)oc4ccccc45)cc3)c3ccccc3-c3cc(-c4ccccc4)c4oc5c(-c6ccccc6)cccc5c4c3)cc2)cc1]

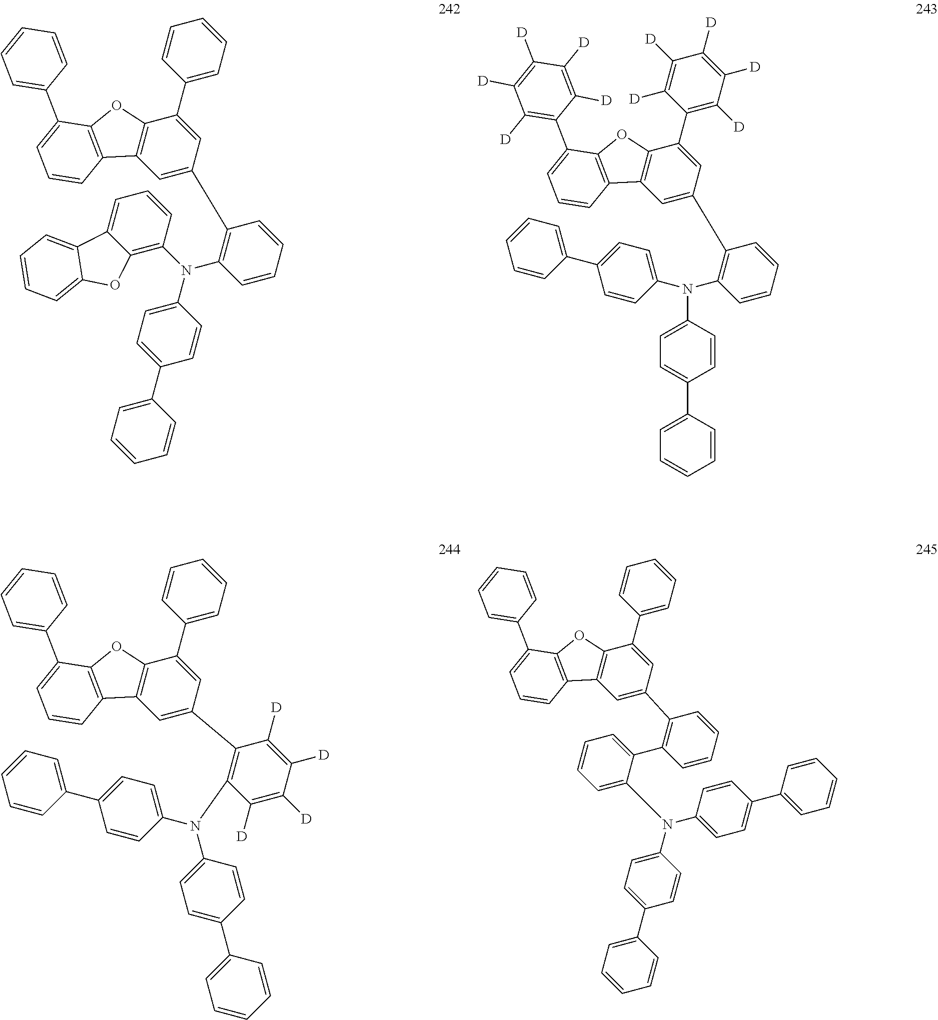 [SMILES: c1ccc(-c2ccc(N(c3ccccc3-c3cc(-c4ccccc4)c4oc5c(-c6ccccc6)cccc5c4c3)c3cccc4c3oc3ccccc34)cc2)cc1 ; [2H]c1c([2H])c([2H])c(-c2cccc3c2oc2c(-c4c([2H])c([2H])c([2H])c([2H])c4[2H])cc(-c4ccccc4N(c4ccc(-c5ccccc5)cc4)c4ccc(-c5ccccc5)cc4)cc23)c([2H])c1[2H] ; [2H]c1c([2H])c([2H])c(N(c2ccc(-c3ccccc3)cc2)c2ccc(-c3ccccc3)cc2)c(-c2cc(-c3ccccc3)c3oc4c(-c5ccccc5)cccc4c3c2)c1[2H] ; c1ccc(-c2ccc(N(c3ccc(-c4ccccc4)cc3)c3ccccc3-c3ccccc3-c3cc(-c4ccccc4)c4oc5c(-c6ccccc6)cccc5c4c3)cc2)cc1]

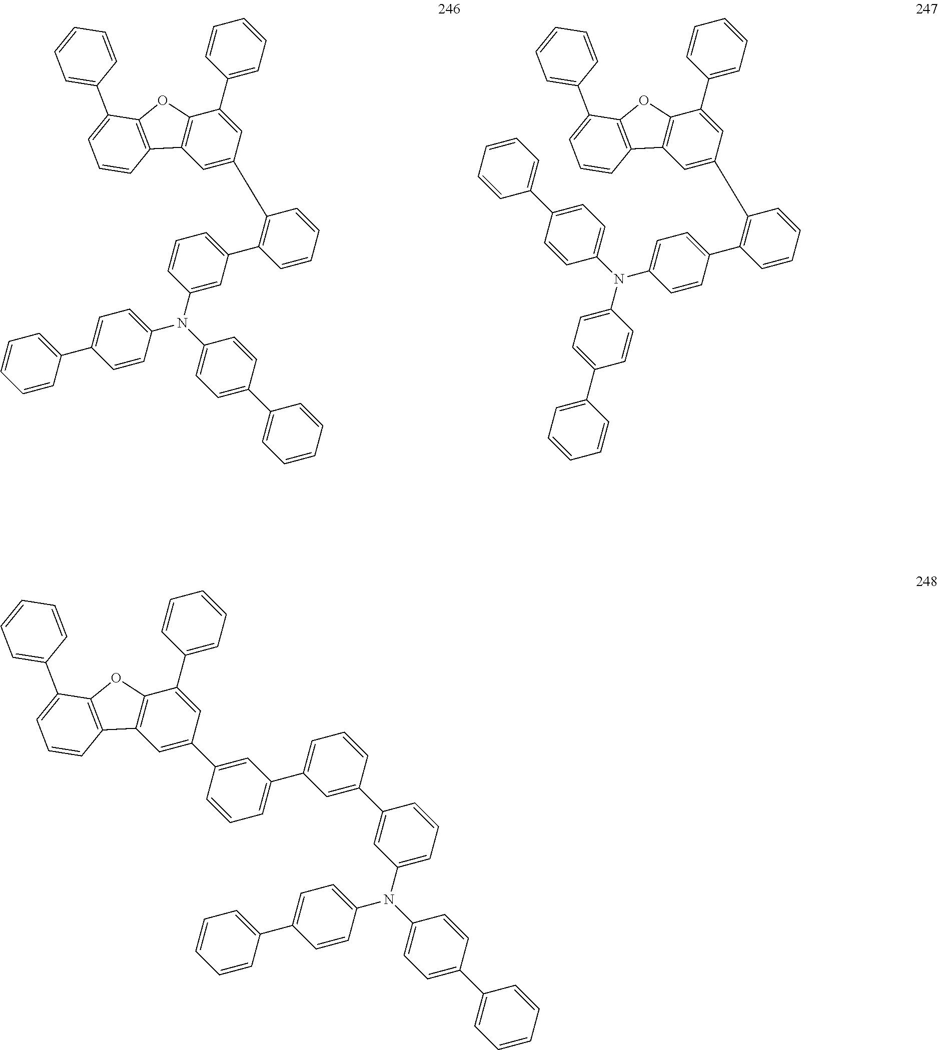 [SMILES: c1ccc(-c2ccc(N(c3ccc(-c4ccccc4)cc3)c3cccc(-c4ccccc4-c4cc(-c5ccccc5)c5oc6c(-c7ccccc7)cccc6c5c4)c3)cc2)cc1 ; c1ccc(-c2ccc(N(c3ccc(-c4ccccc4)cc3)c3ccc(-c4ccccc4-c4cc(-c5ccccc5)c5oc6c(-c7ccccc7)cccc6c5c4)cc3)cc2)cc1 ; c1ccc(-c2ccc(N(c3ccc(-c4ccccc4)cc3)c3cccc(-c4cccc(-c5cccc(-c6cc(-c7ccccc7)c7oc8c(-c9ccccc9)cccc8c7c6)c5)c4)c3)cc2)cc1]

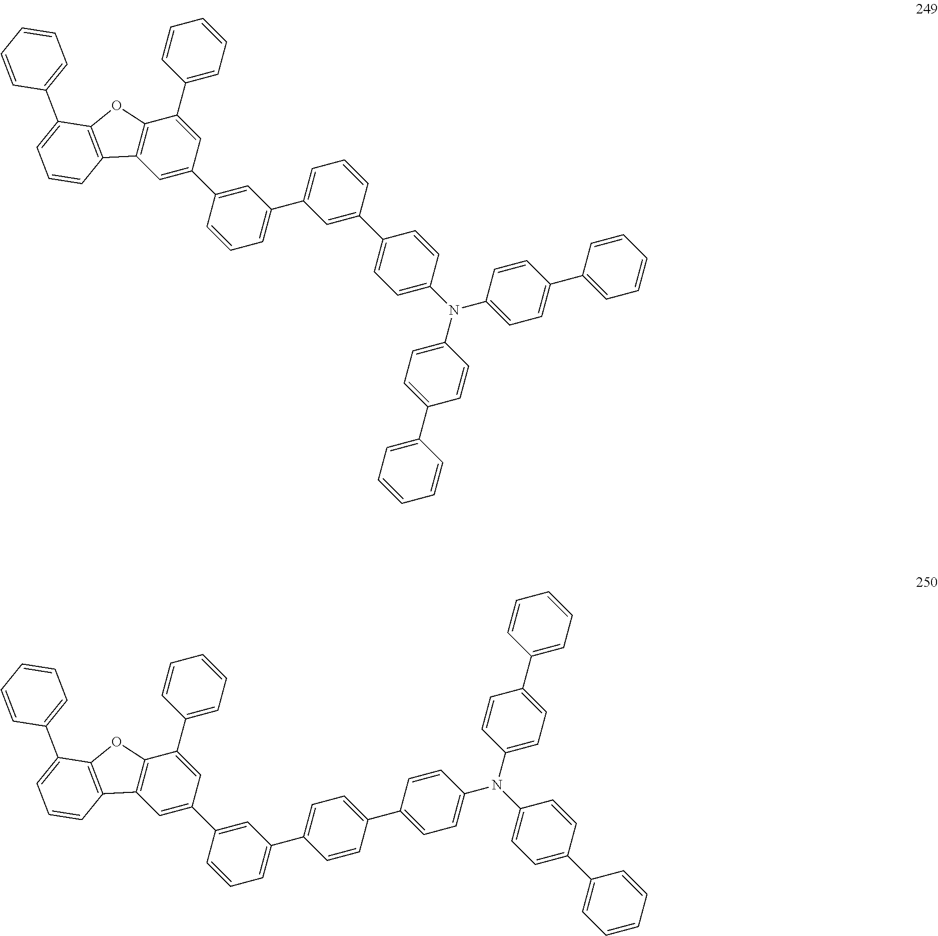 [SMILES: c1ccc(-c2ccc(N(c3ccc(-c4ccccc4)cc3)c3ccc(-c4cccc(-c5cccc(-c6cc(-c7ccccc7)c7oc8c(-c9ccccc9)cccc8c7c6)c5)c4)cc3)cc2)cc1 ; c1ccc(-c2ccc(N(c3ccc(-c4ccccc4)cc3)c3ccc(-c4ccc(-c5cccc(-c6cc(-c7ccccc7)c7oc8c(-c9ccccc9)cccc8c7c6)c5)cc4)cc3)cc2)cc1]

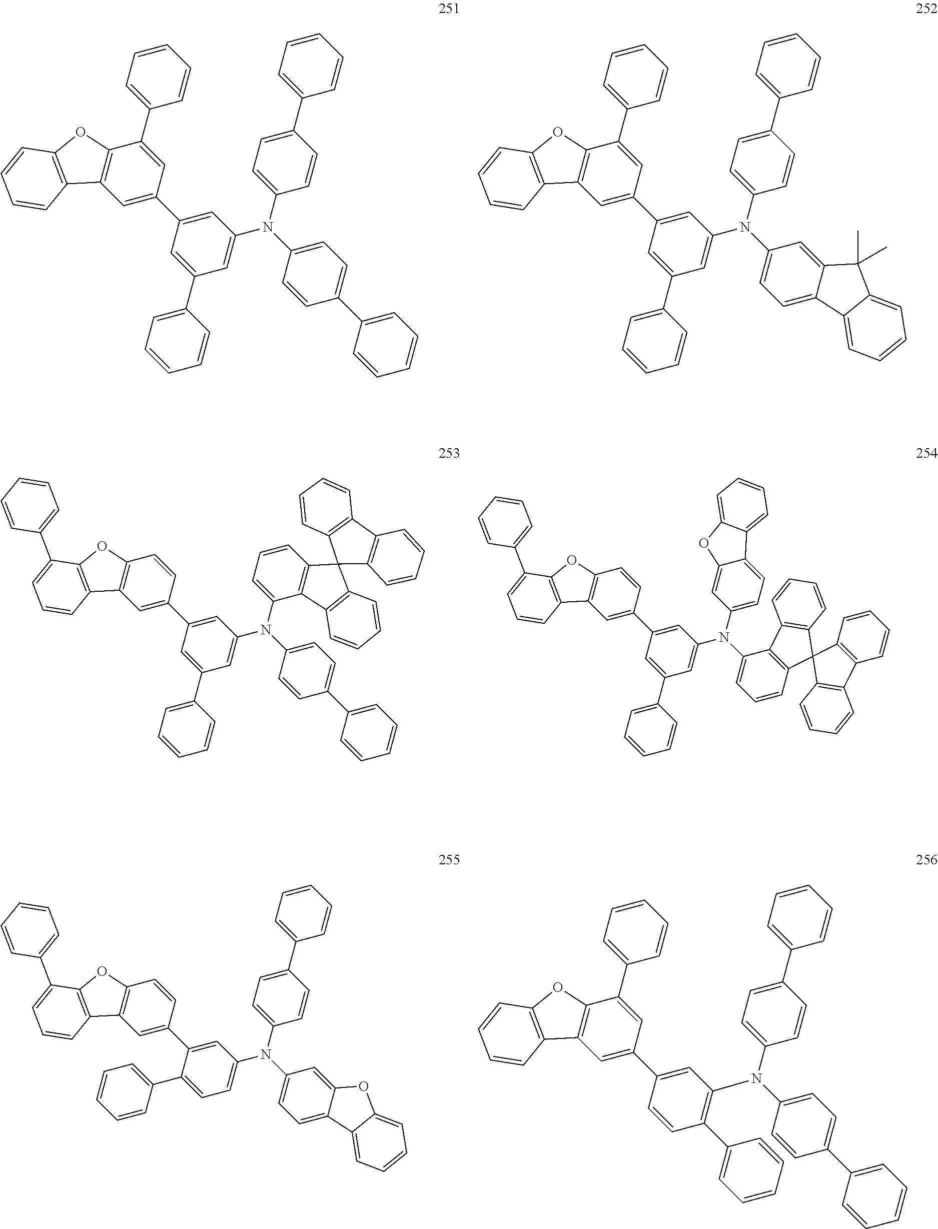

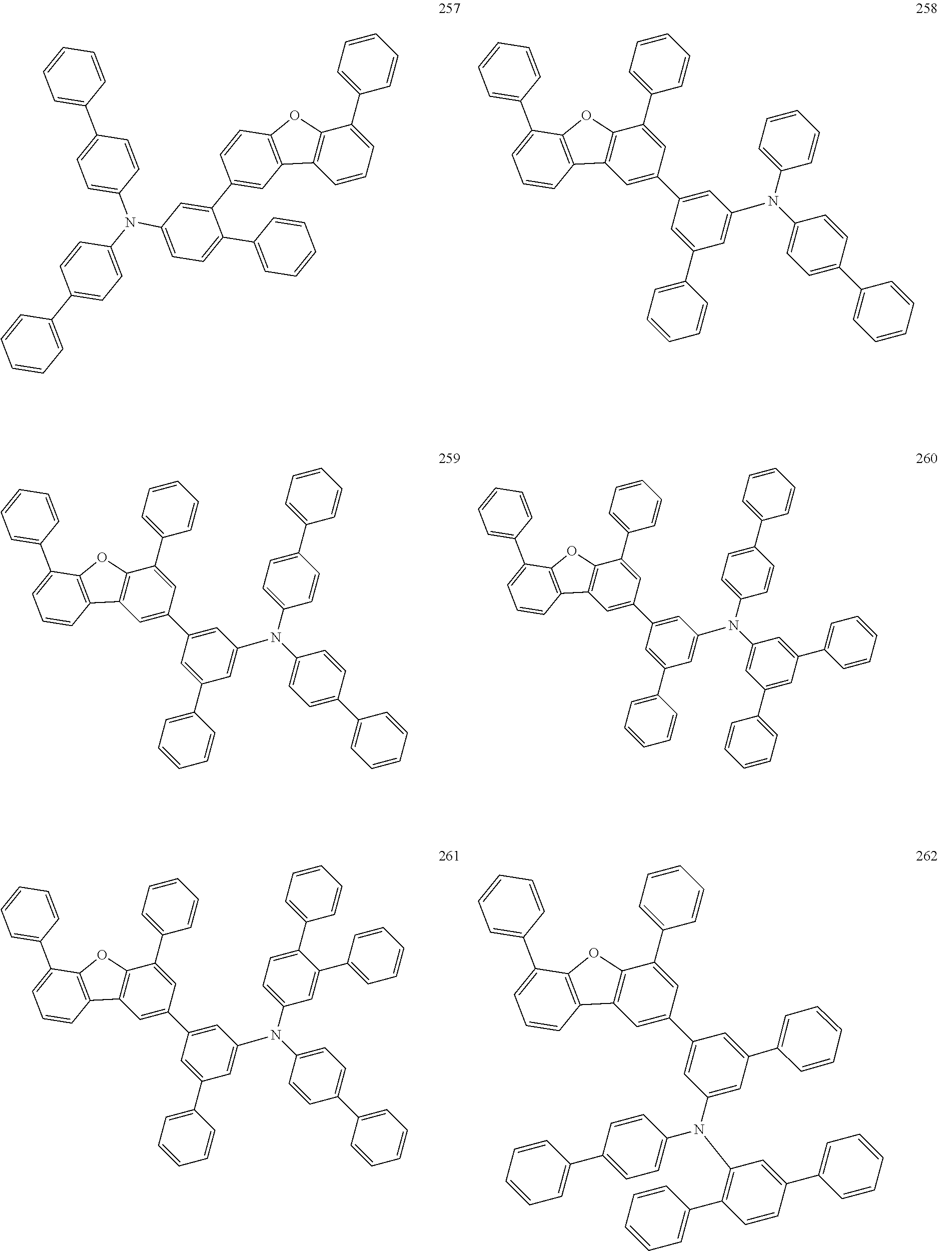

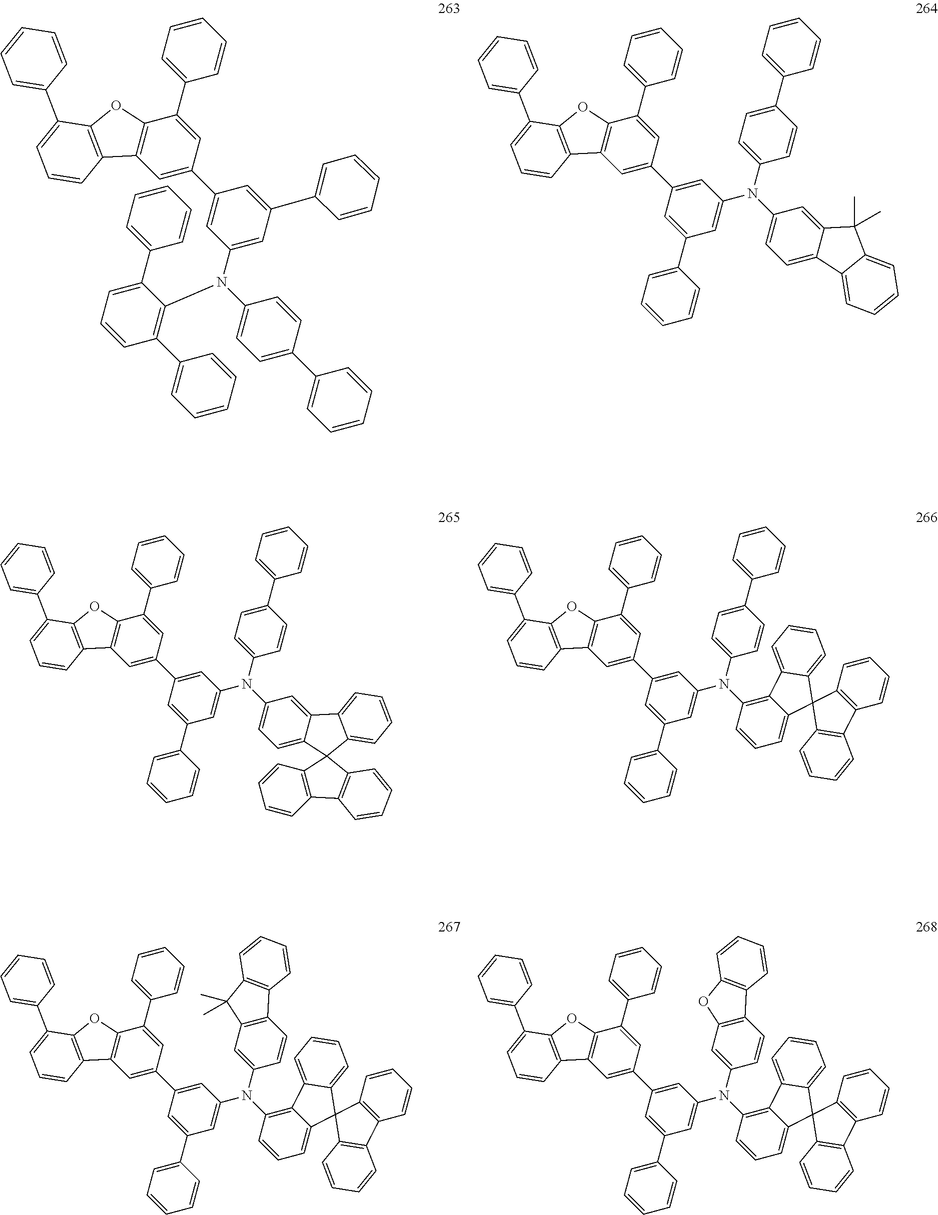

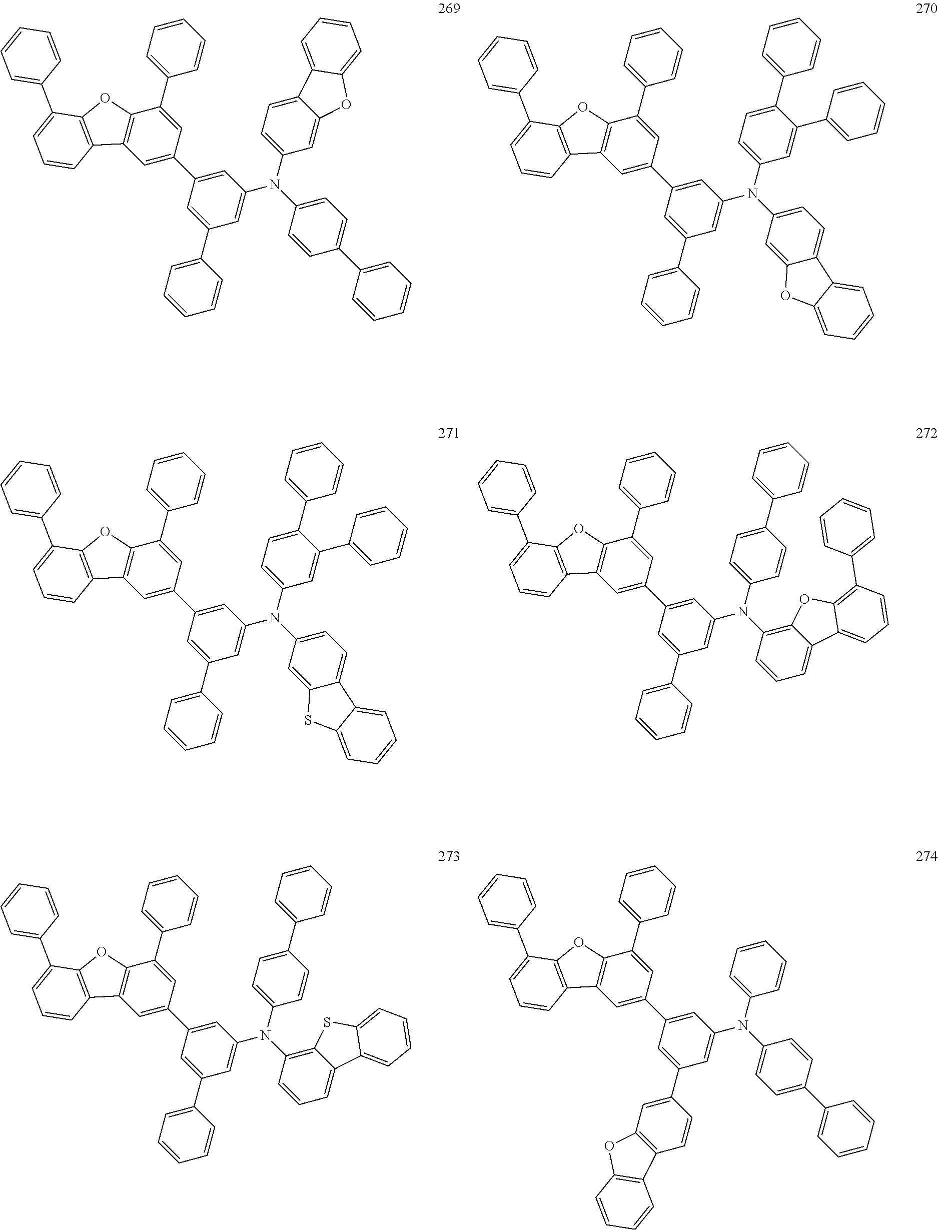

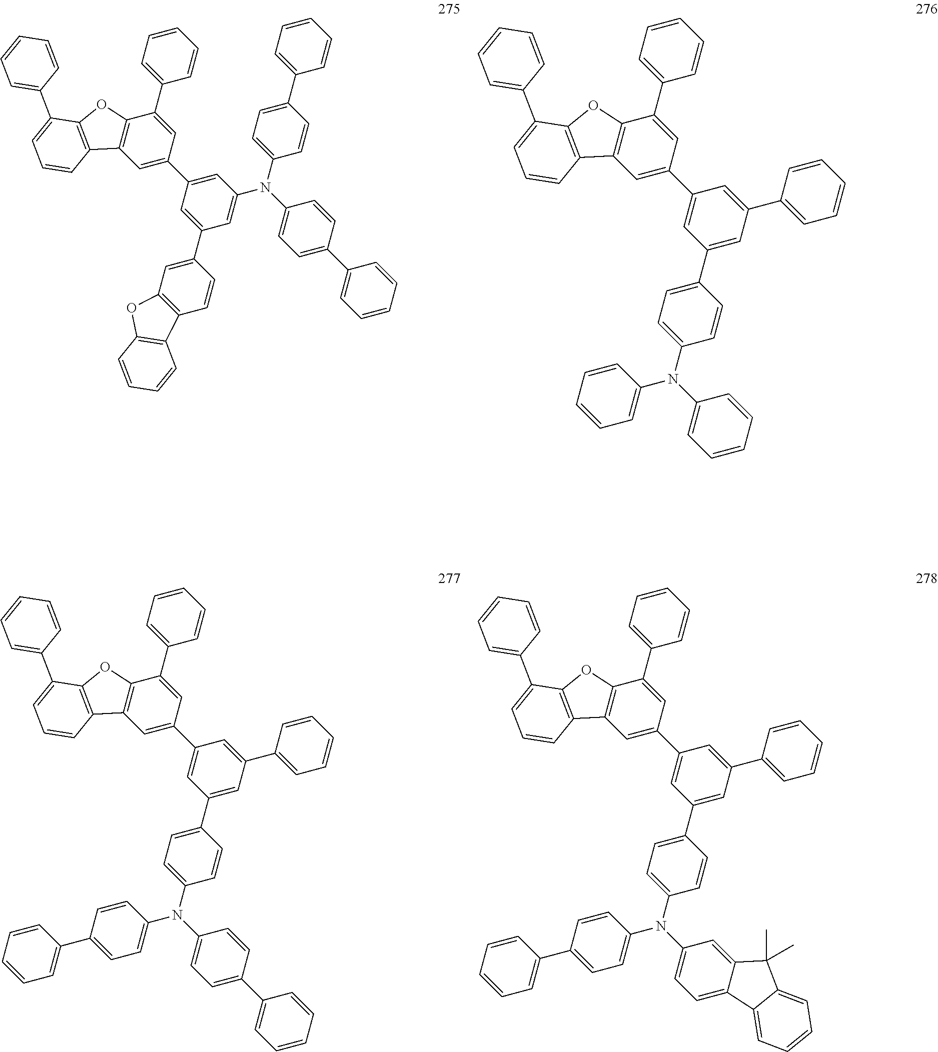 [SMILES: c1ccc(-c2ccc(N(c3ccc(-c4ccccc4)cc3)c3cc(-c4ccc5c(c4)oc4ccccc45)cc(-c4cc(-c5ccccc5)c5oc6c(-c7ccccc7)cccc6c5c4)c3)cc2)cc1 ; c1ccc(-c2cc(-c3ccc(N(c4ccccc4)c4ccccc4)cc3)cc(-c3cc(-c4ccccc4)c4oc5c(-c6ccccc6)cccc5c4c3)c2)cc1 ; c1ccc(-c2ccc(N(c3ccc(-c4ccccc4)cc3)c3ccc(-c4cc(-c5ccccc5)cc(-c5cc(-c6ccccc6)c6oc7c(-c8ccccc8)cccc7c6c5)c4)cc3)cc2)cc1 ; CC1(C)c2ccccc2-c2ccc(N(c3ccc(-c4ccccc4)cc3)c3ccc(-c4cc(-c5ccccc5)cc(-c5cc(-c6ccccc6)c6oc7c(-c8ccccc8)cccc7c6c5)c4)cc3)cc21]

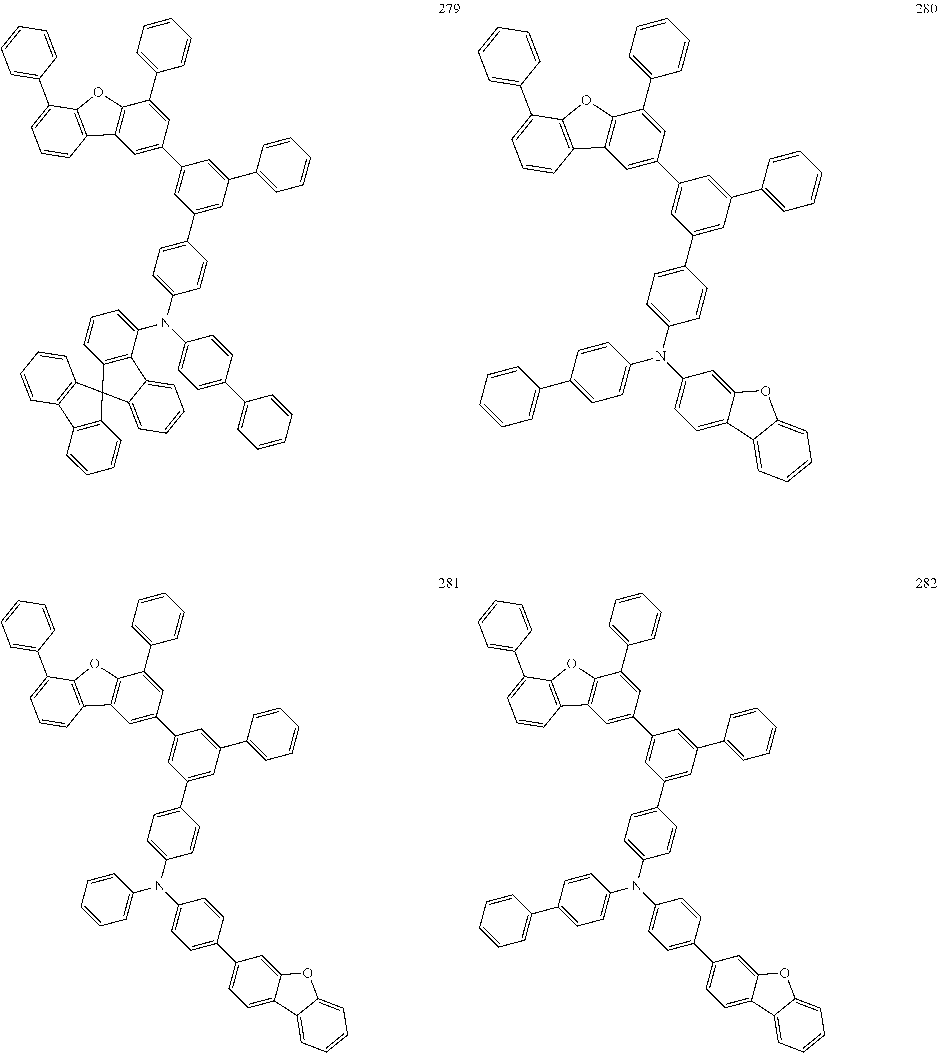

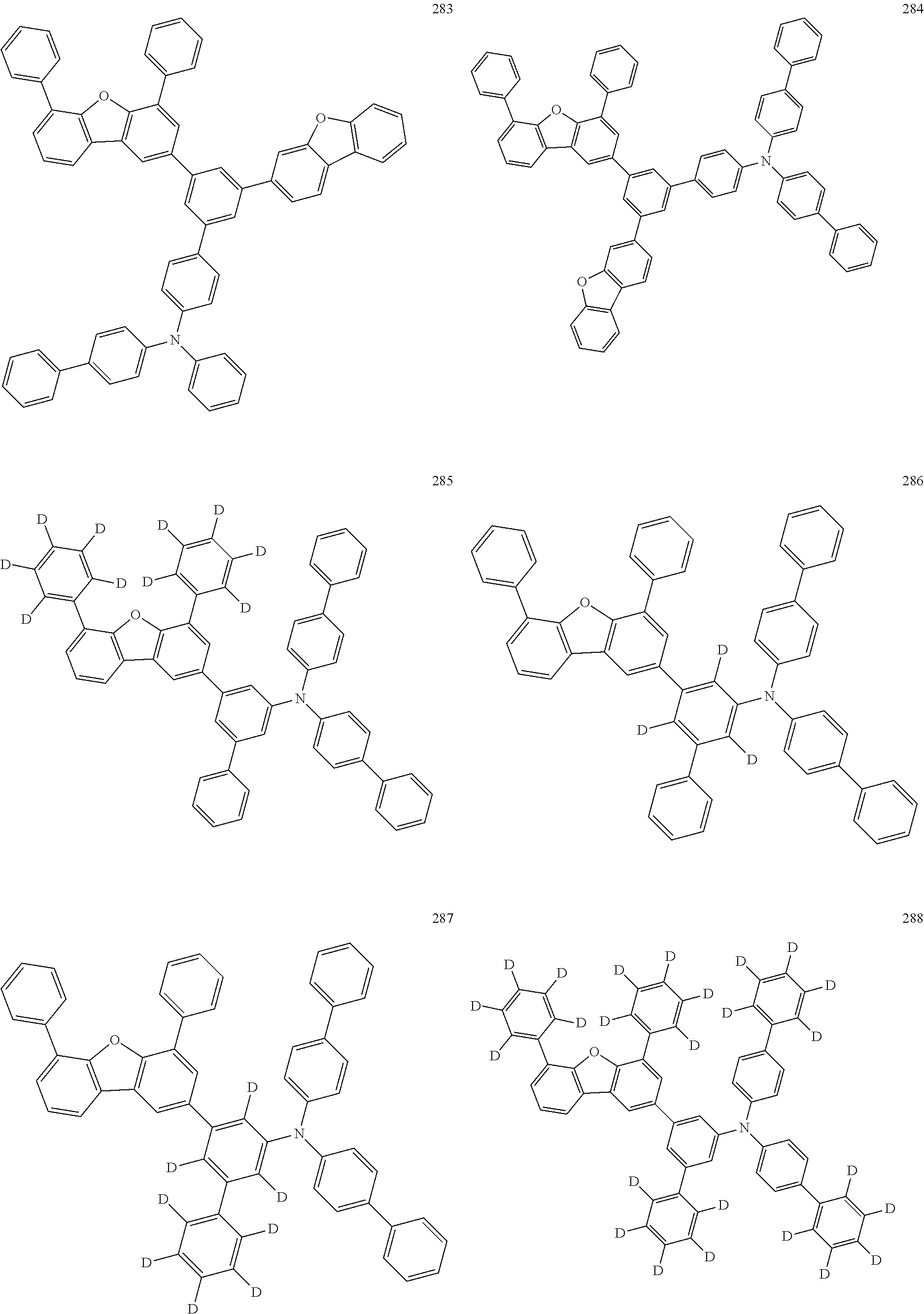

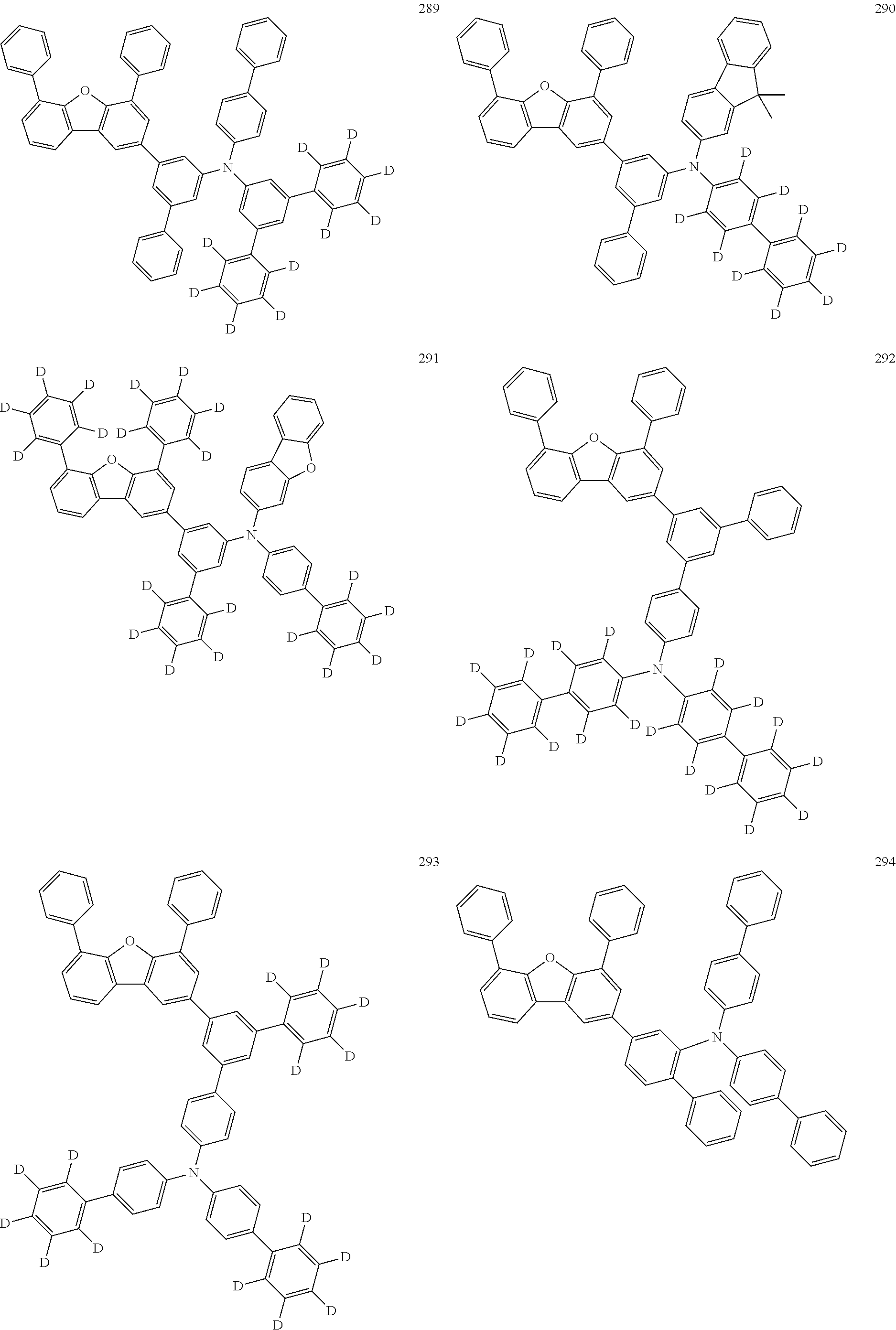

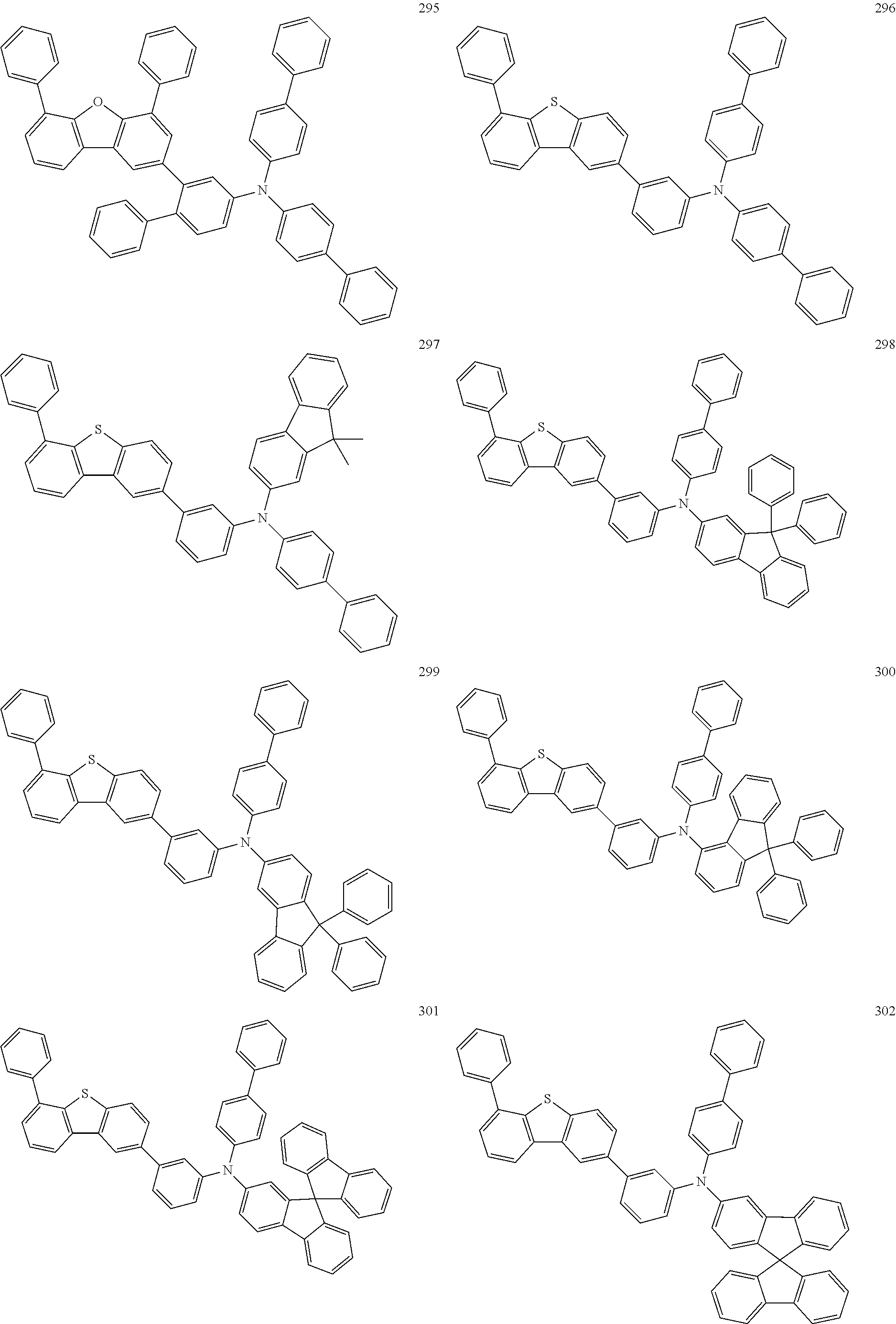

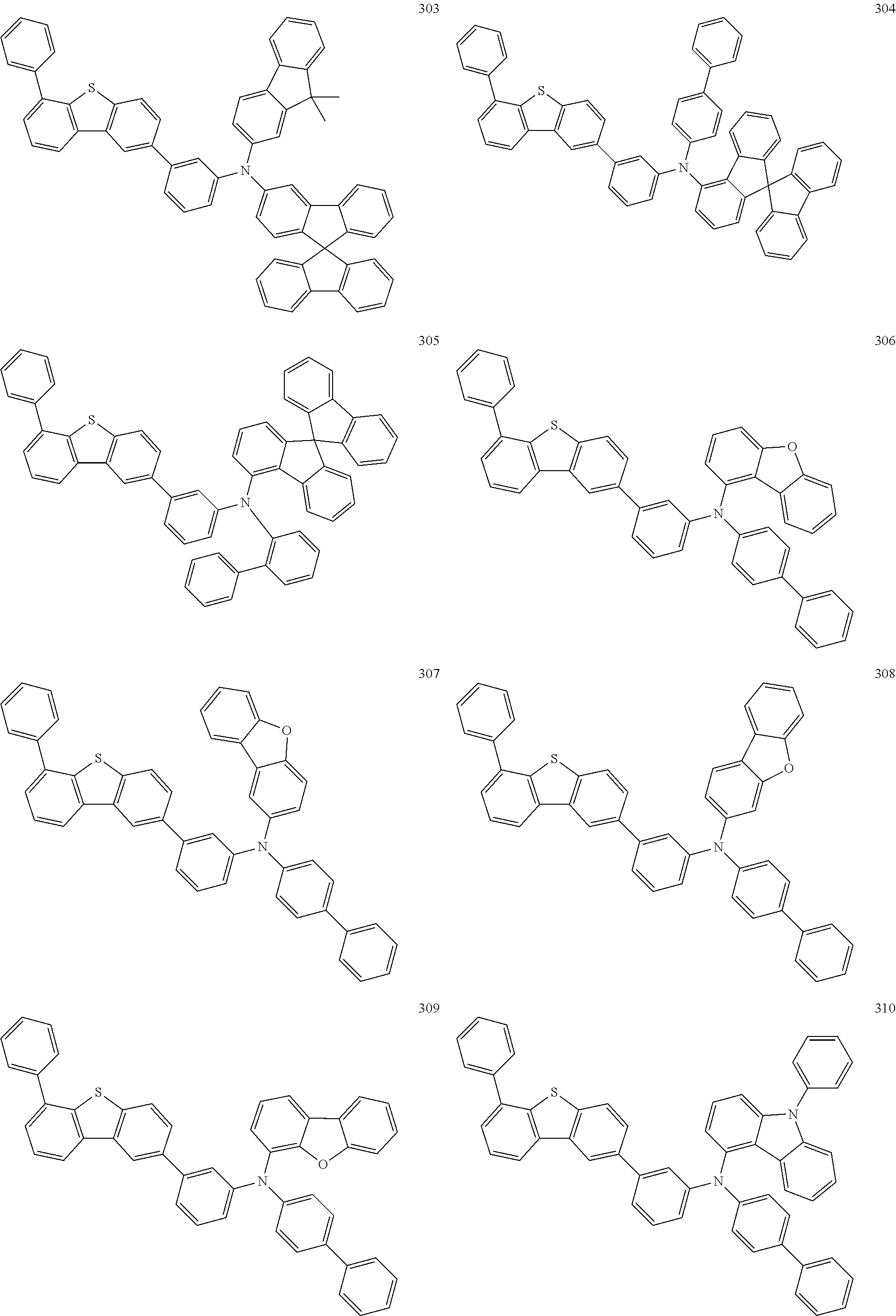

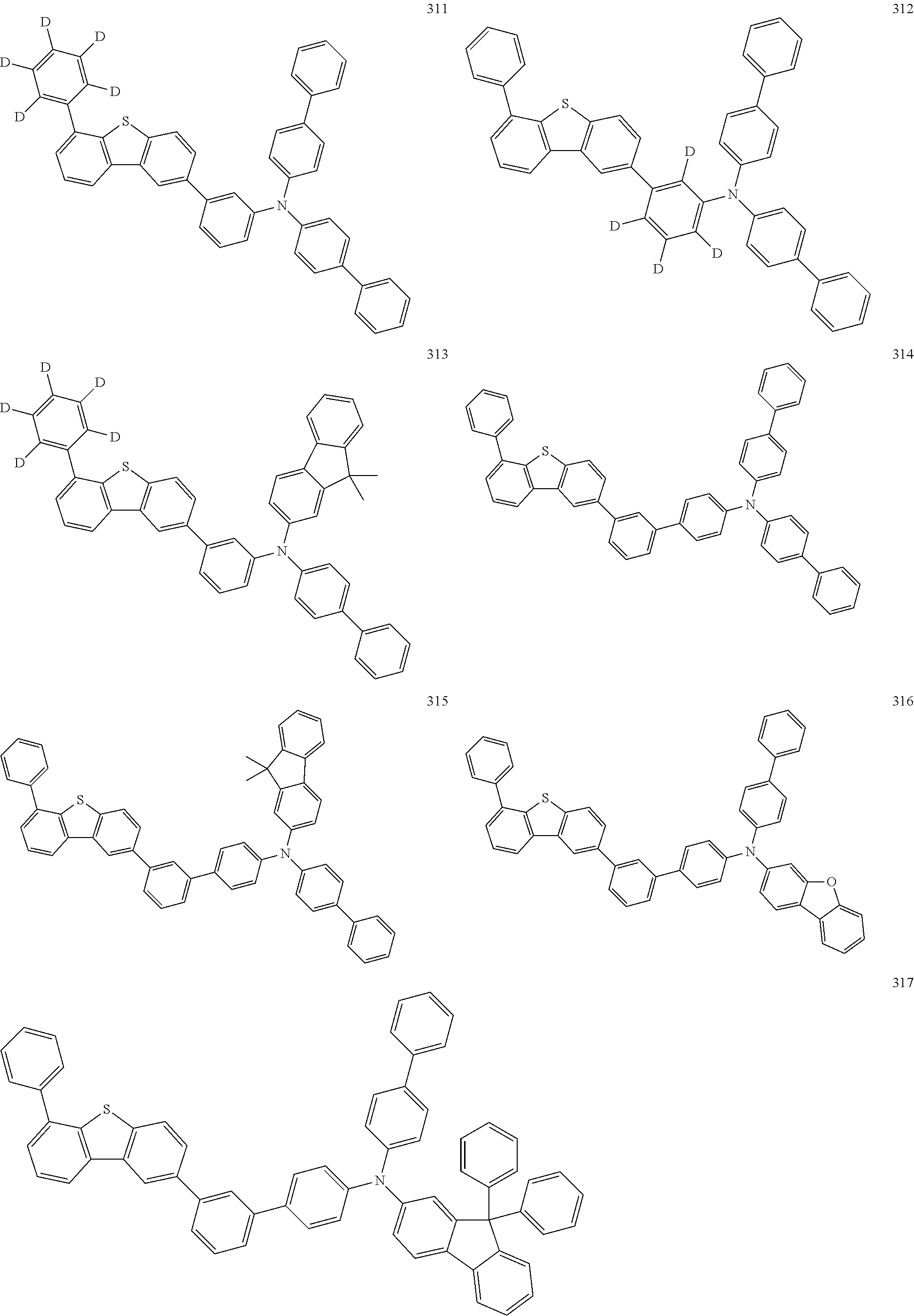

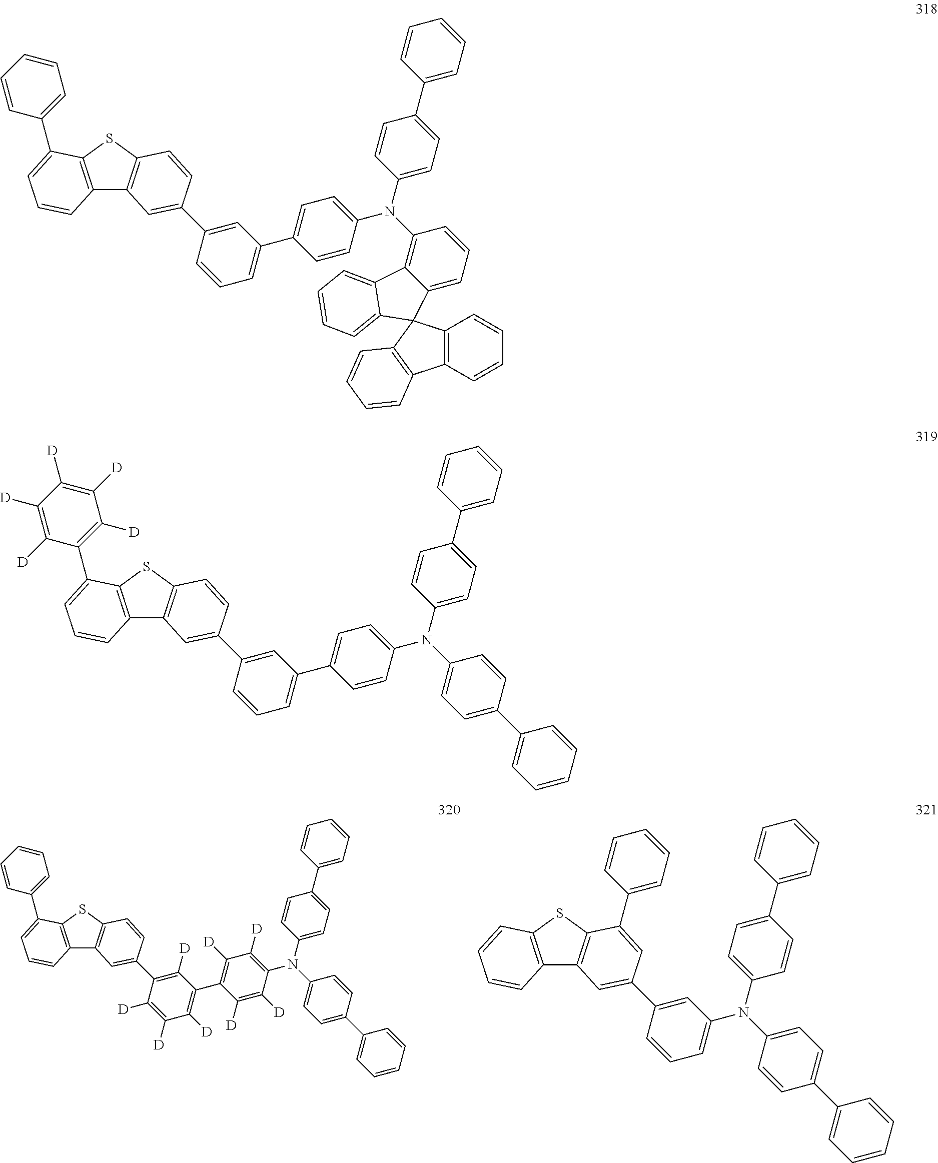 [SMILES: c1ccc(-c2ccc(N(c3ccc(-c4cccc(-c5ccc6sc7c(-c8ccccc8)cccc7c6c5)c4)cc3)c3cccc4c3-c3ccccc3C43c4ccccc4-c4ccccc43)cc2)cc1 ; [2H]c1c([2H])c([2H])c(-c2cccc3c2sc2ccc(-c4cccc(-c5ccc(N(c6ccc(-c7ccccc7)cc6)c6ccc(-c7ccccc7)cc6)cc5)c4)cc23)c([2H])c1[2H] ; [2H]c1c([2H])c(-c2ccc3sc4c(-c5ccccc5)cccc4c3c2)c([2H])c(-c2c([2H])c([2H])c(N(c3ccc(-c4ccccc4)cc3)c3ccc(-c4ccccc4)cc3)c([2H])c2[2H])c1[2H] ; c1ccc(-c2ccc(N(c3ccc(-c4ccccc4)cc3)c3cccc(-c4cc(-c5ccccc5)c5sc6ccccc6c5c4)c3)cc2)cc1]

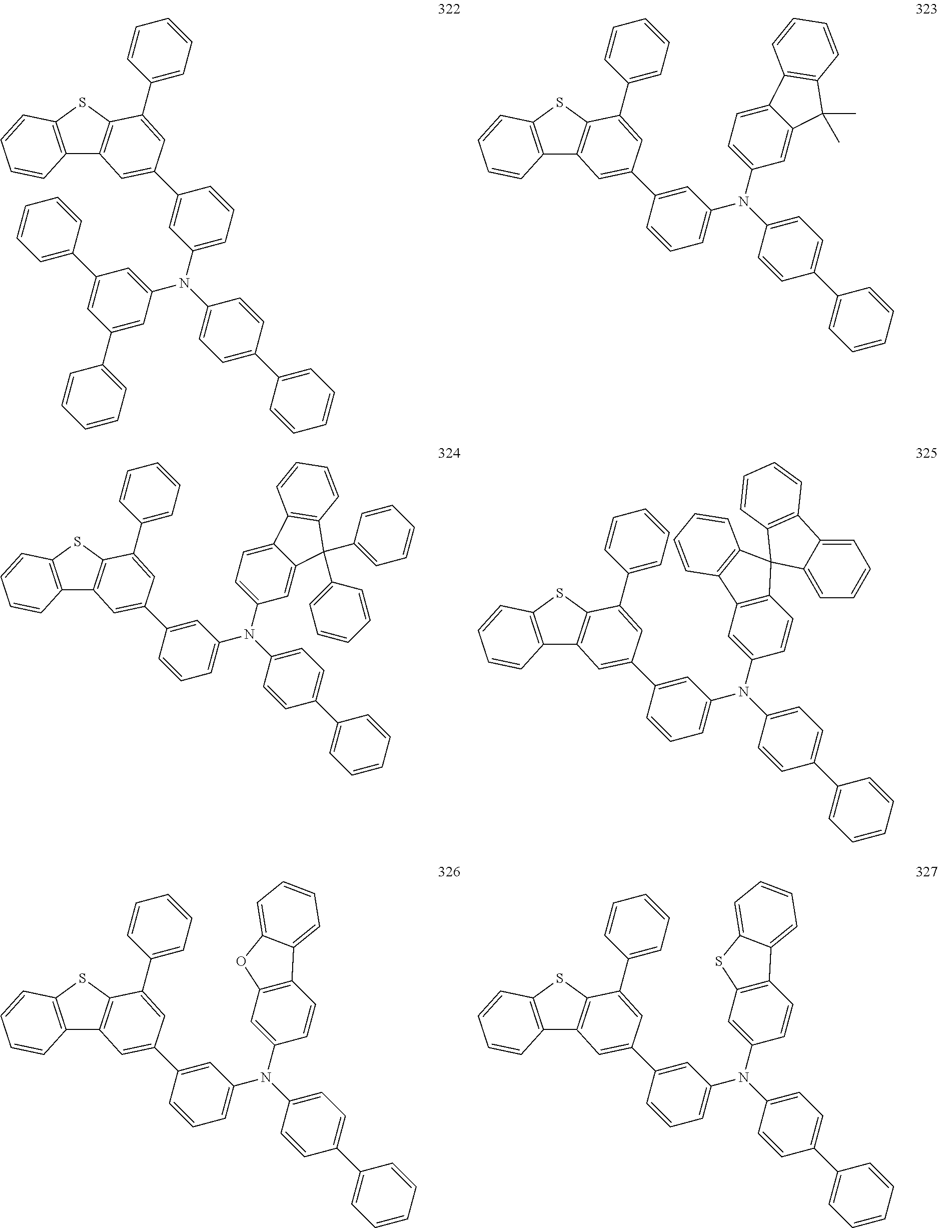

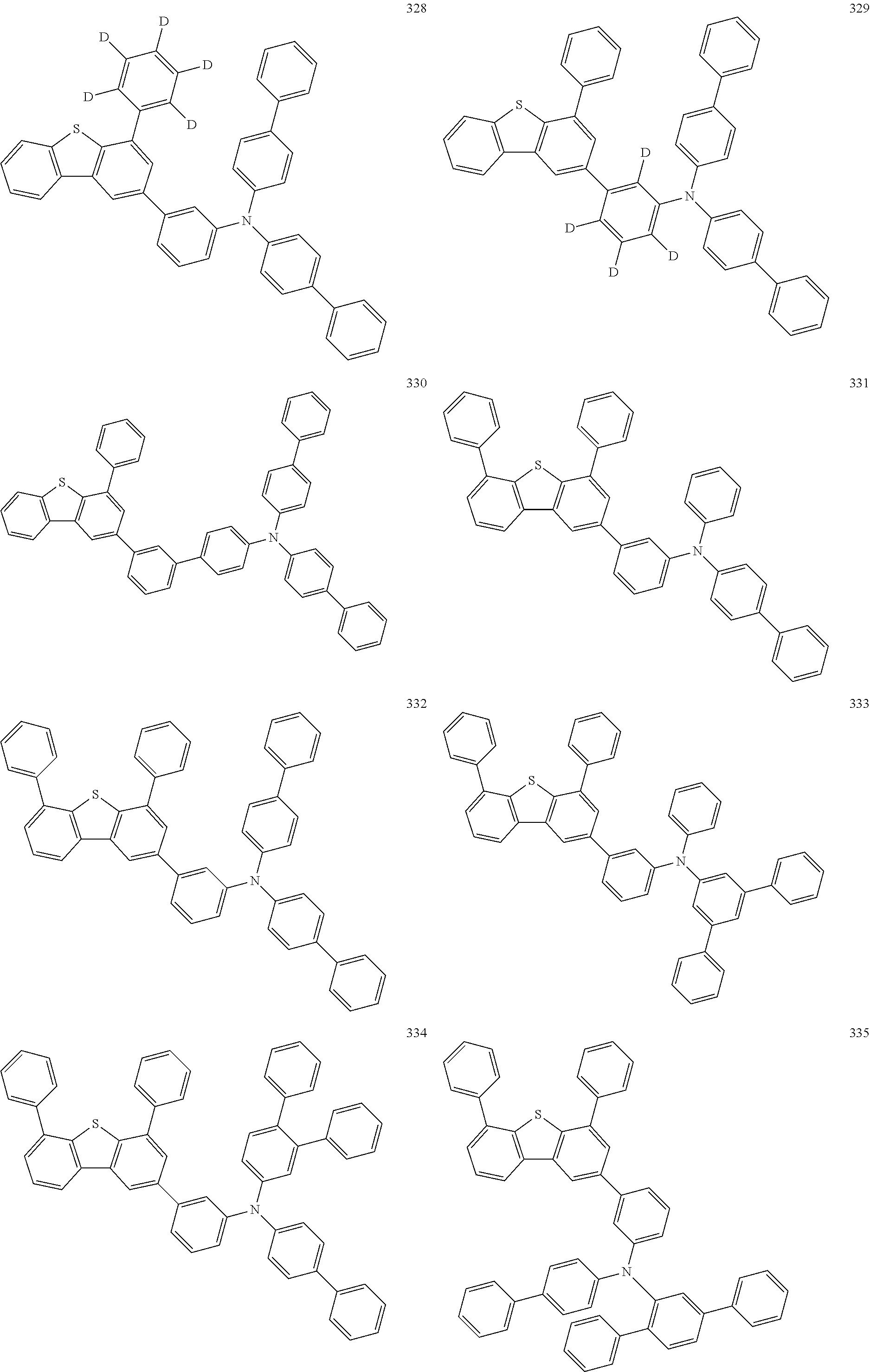

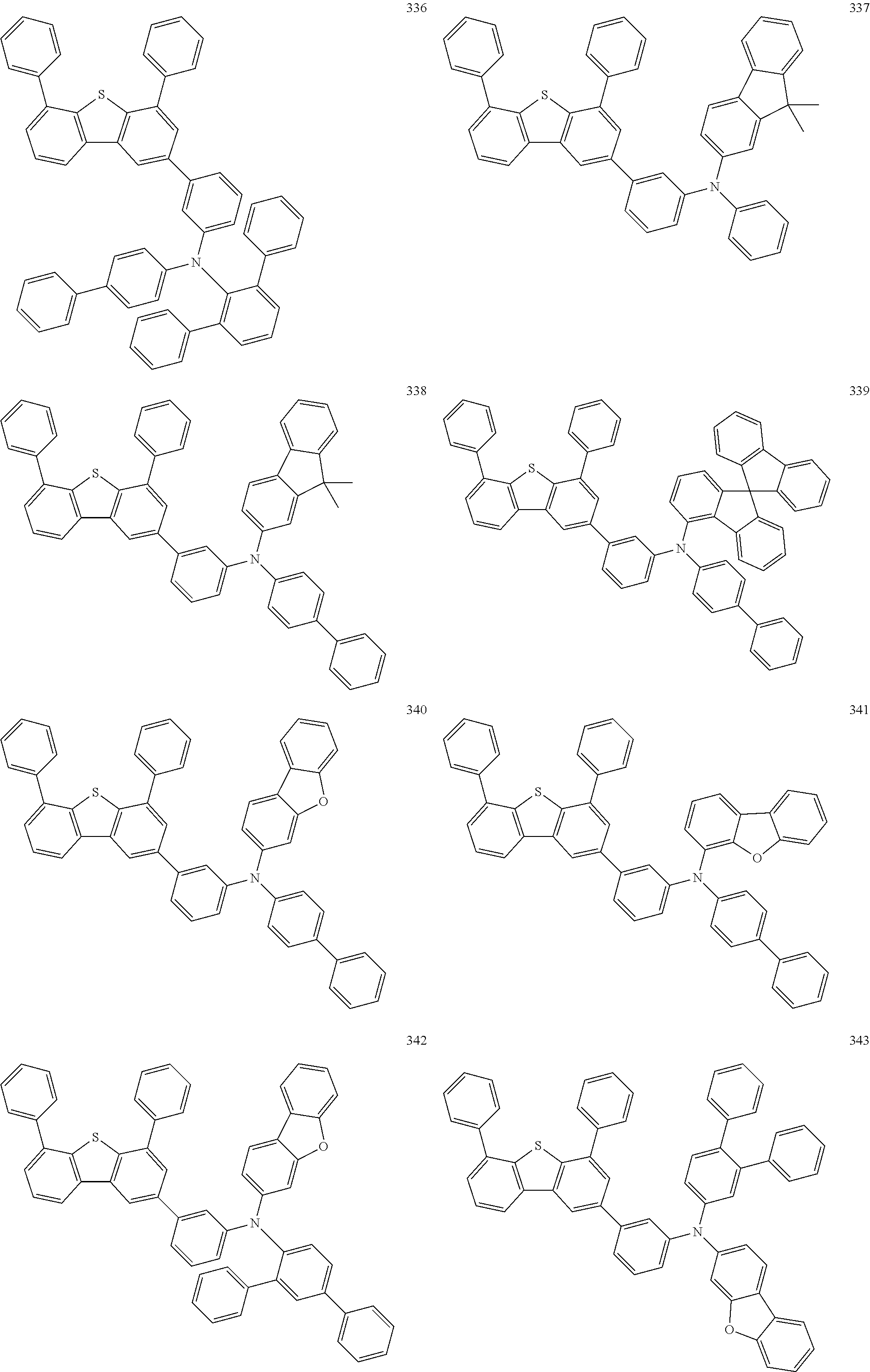

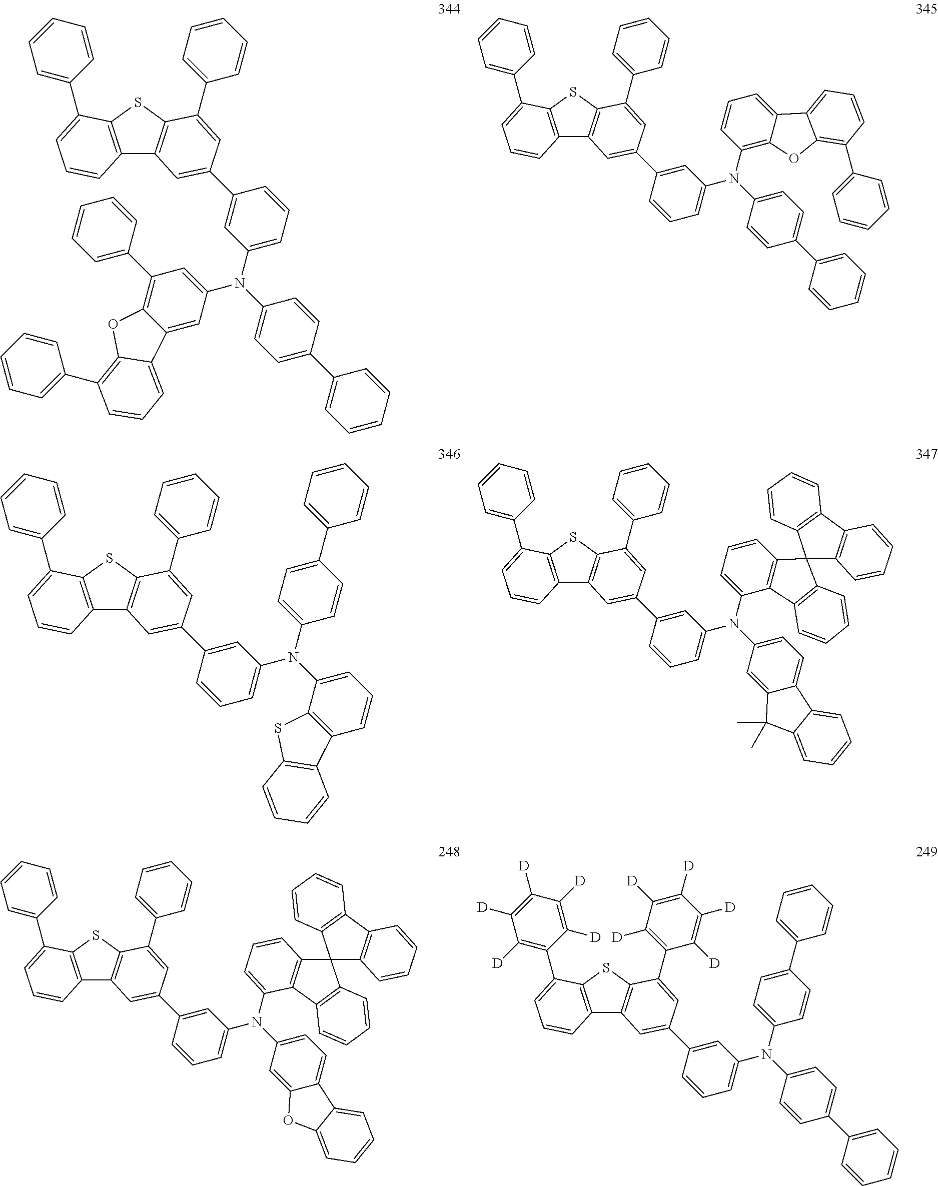

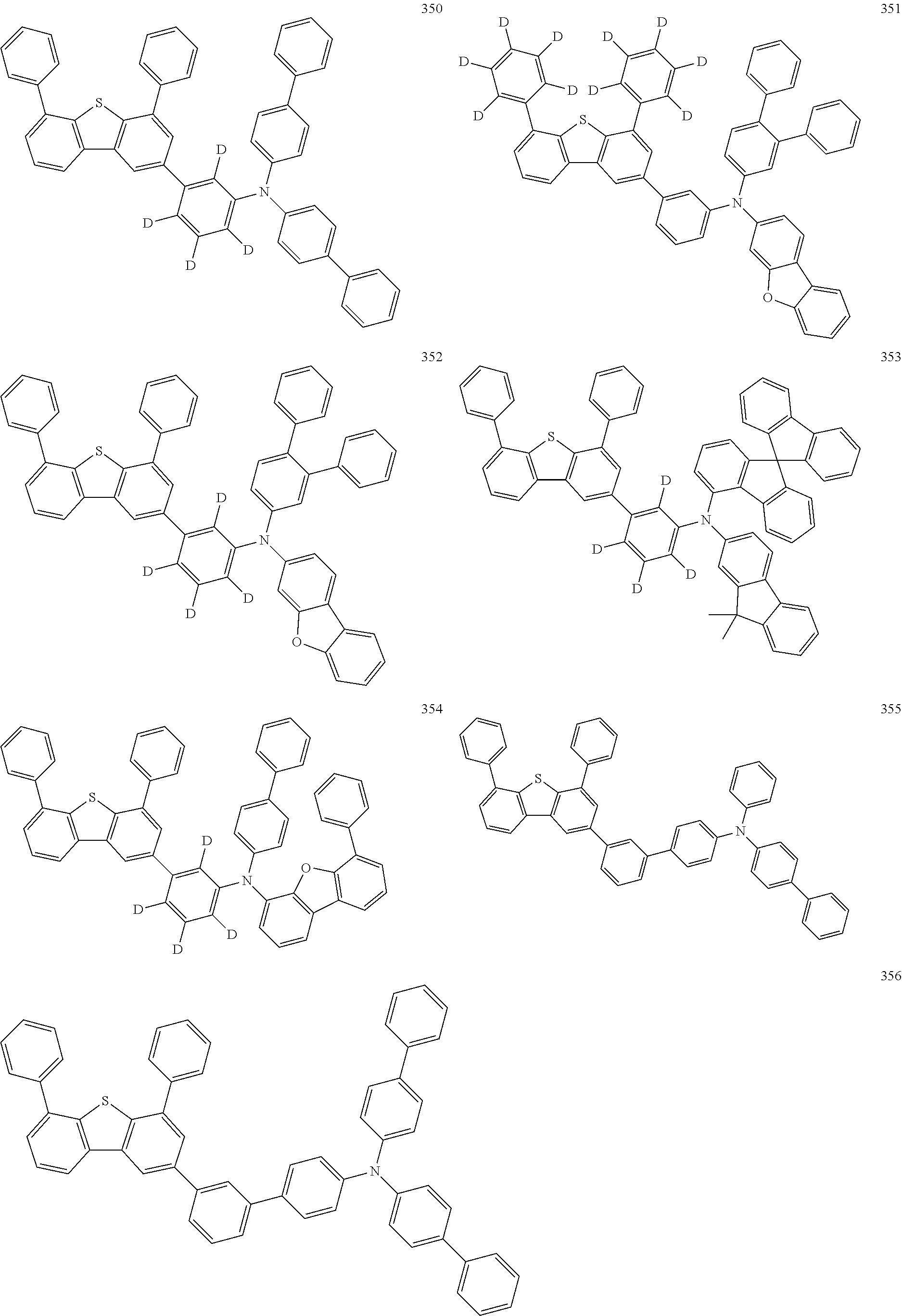

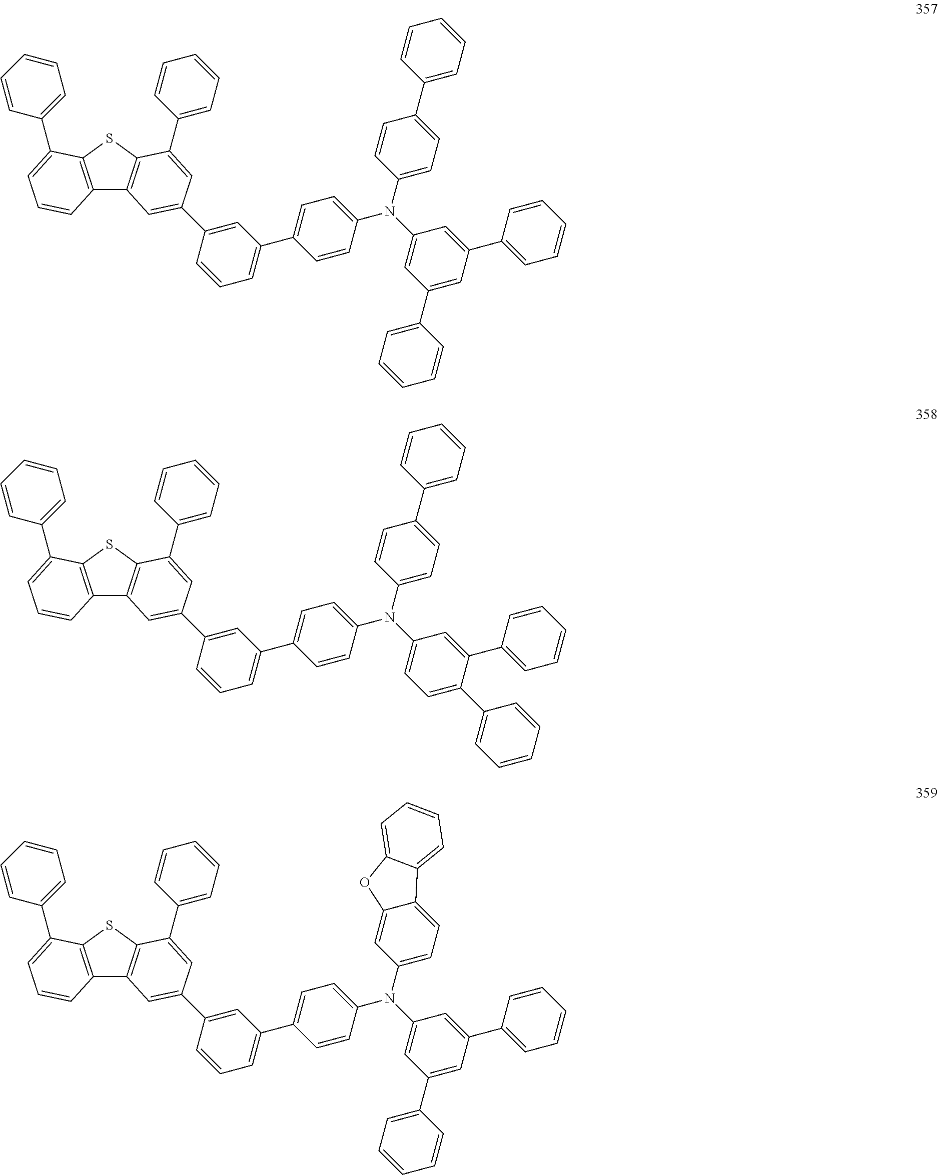

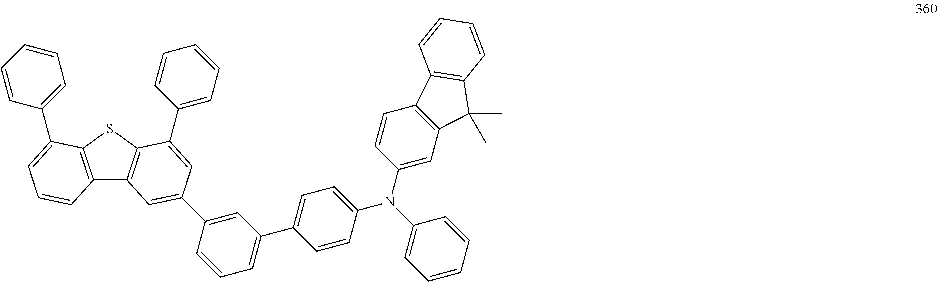 [SMILES: CC1(C)c2ccccc2-c2ccc(N(c3ccccc3)c3ccc(-c4cccc(-c5cc(-c6ccccc6)c6sc7c(-c8ccccc8)cccc7c6c5)c4)cc3)cc21]

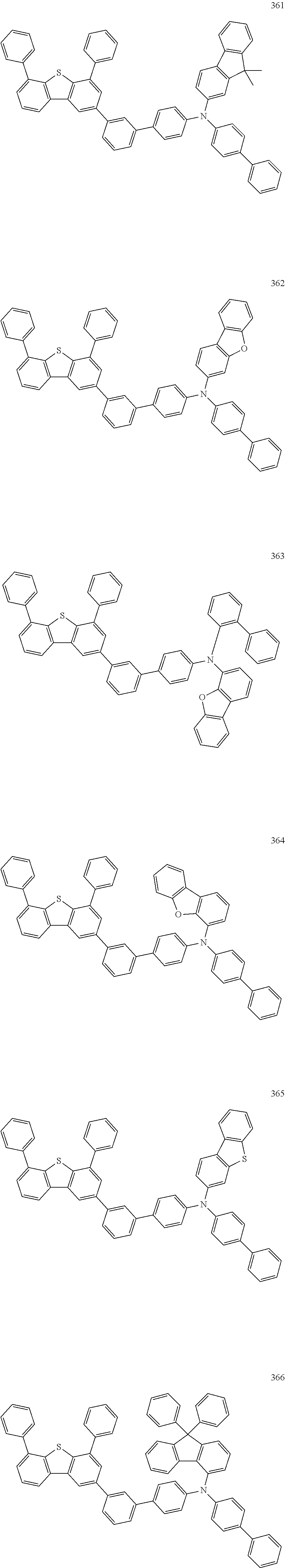

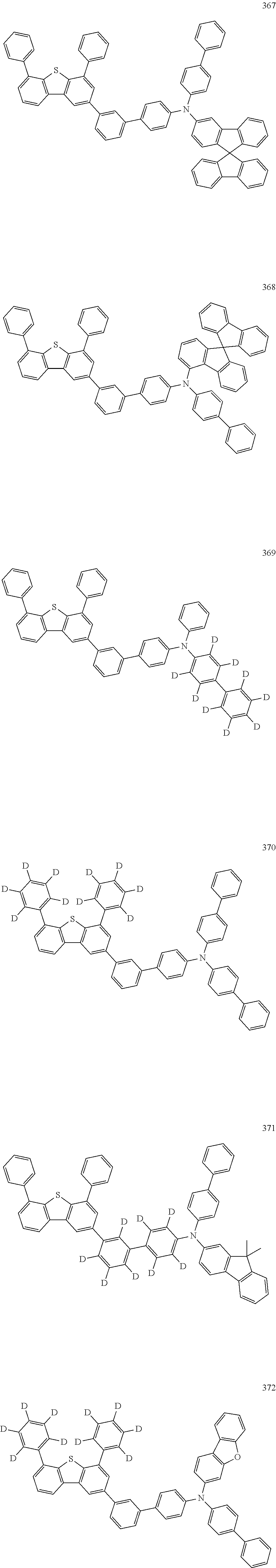

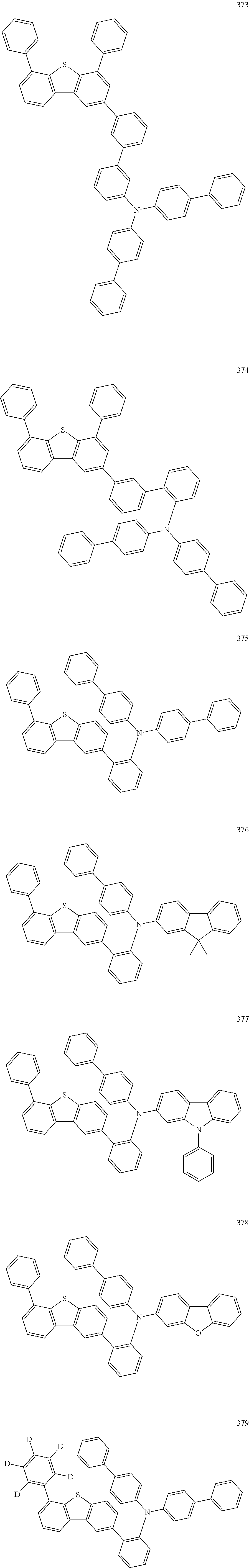

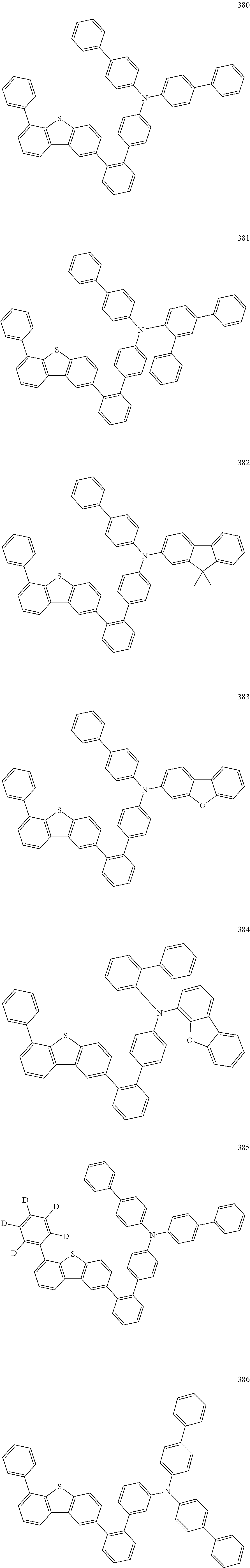

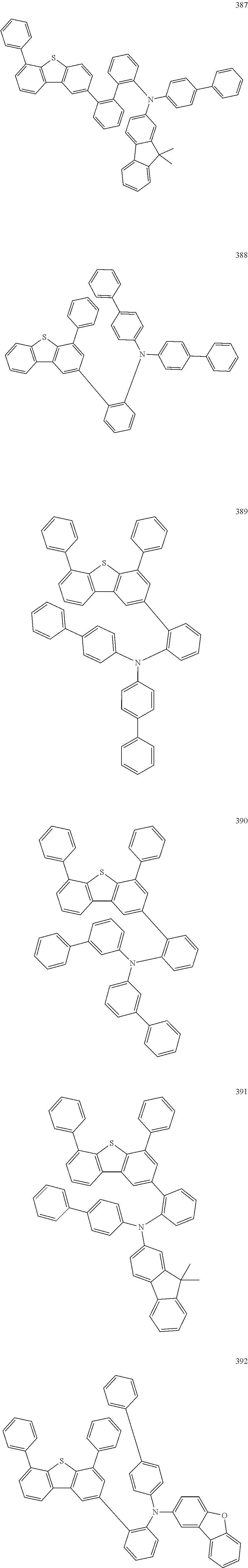

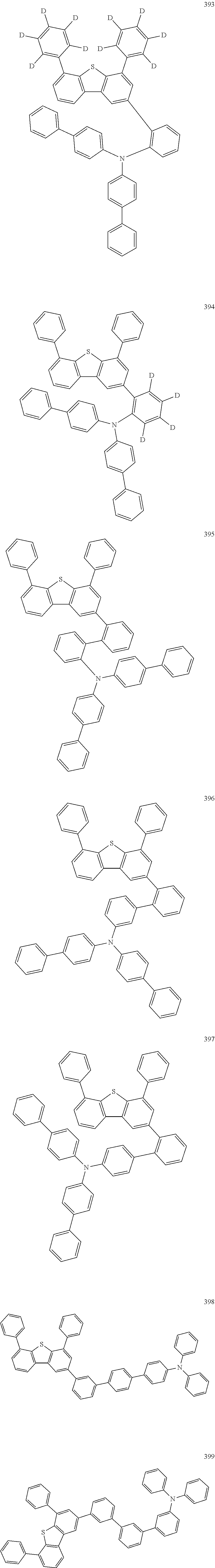

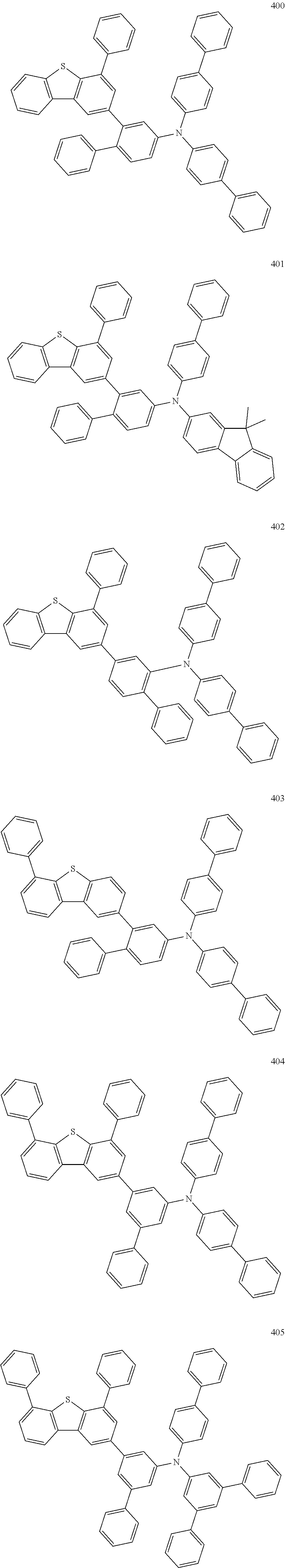

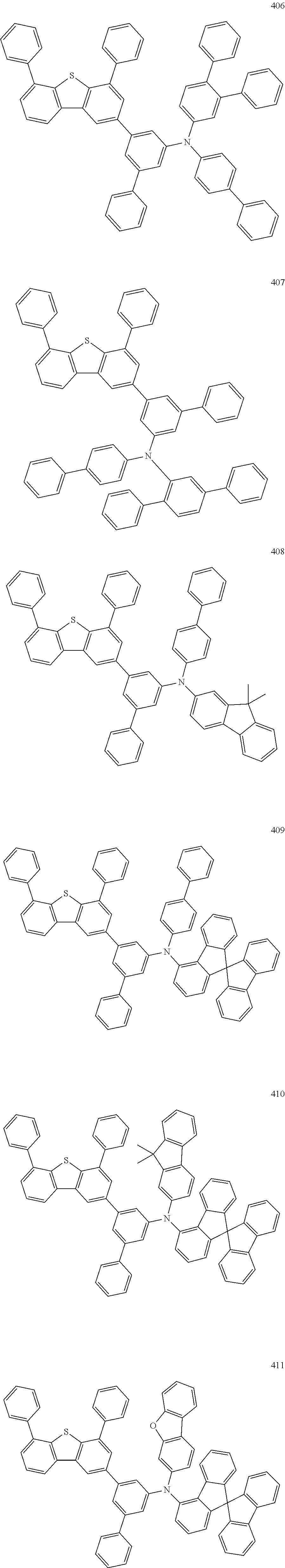

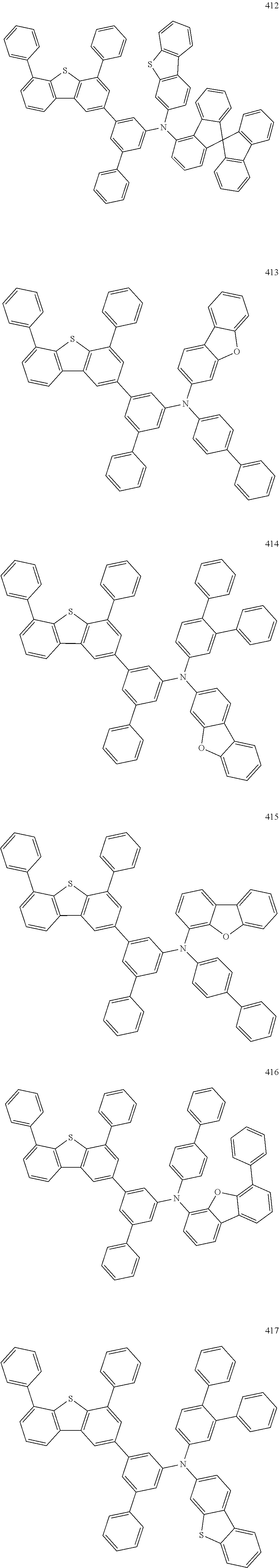

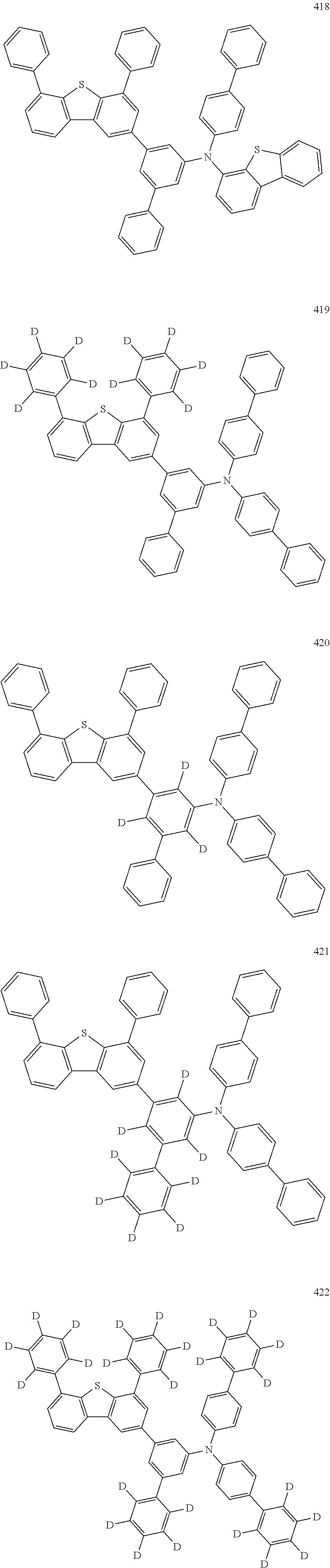

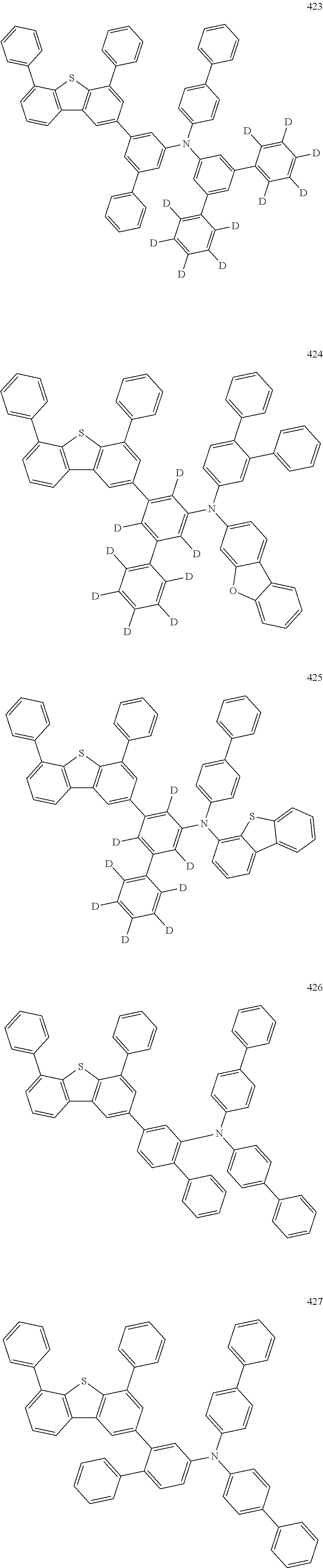

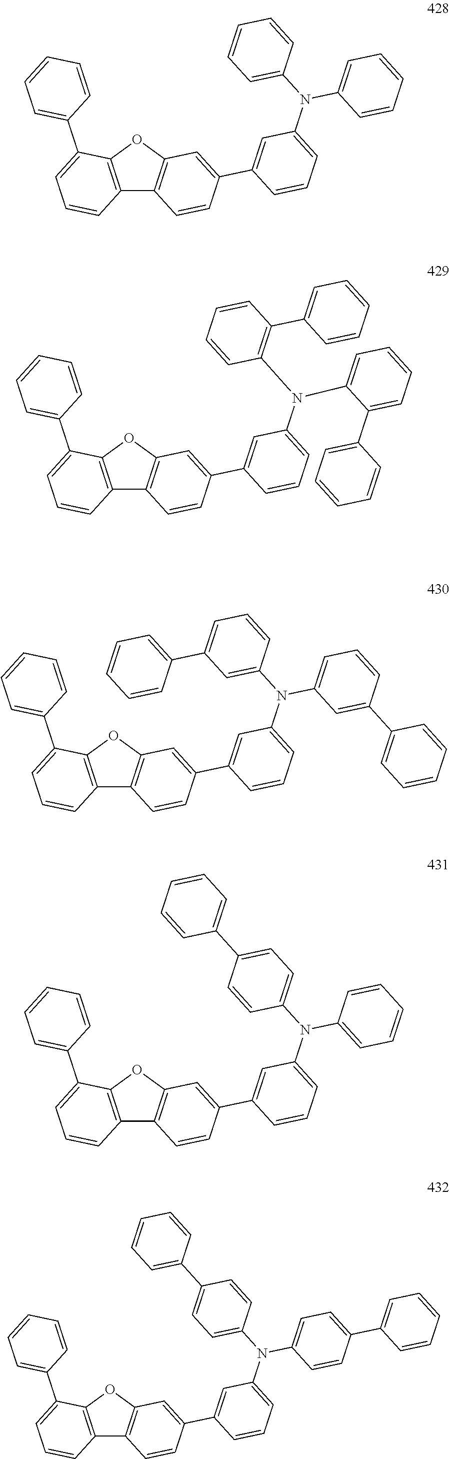 [SMILES: c1ccc(-c2cccc3c2oc2cc(-c4cccc(N(c5ccccc5)c5ccccc5)c4)ccc23)cc1 ; c1ccc(-c2ccccc2N(c2cccc(-c3ccc4c(c3)oc3c(-c5ccccc5)cccc34)c2)c2ccccc2-c2ccccc2)cc1 ; c1ccc(-c2cccc(N(c3cccc(-c4ccccc4)c3)c3cccc(-c4ccc5c(c4)oc4c(-c6ccccc6)cccc45)c3)c2)cc1 ; c1ccc(-c2ccc(N(c3ccccc3)c3cccc(-c4ccc5c(c4)oc4c(-c6ccccc6)cccc45)c3)cc2)cc1 ; c1ccc(-c2ccc(N(c3ccc(-c4ccccc4)cc3)c3cccc(-c4ccc5c(c4)oc4c(-c6ccccc6)cccc45)c3)cc2)cc1]

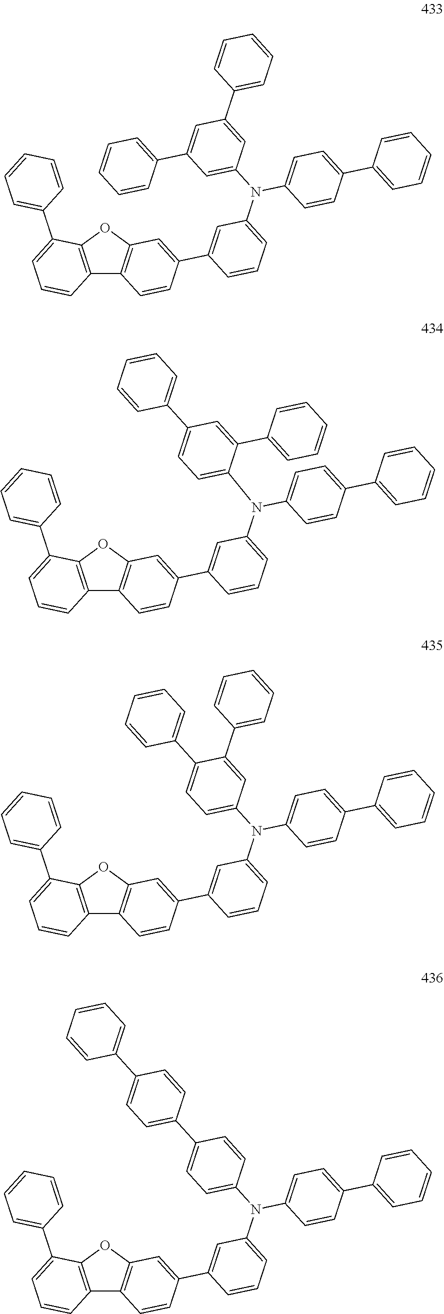 [SMILES: c1ccc(-c2ccc(N(c3cccc(-c4ccc5c(c4)oc4c(-c6ccccc6)cccc45)c3)c3cc(-c4ccccc4)cc(-c4ccccc4)c3)cc2)cc1 ; c1ccc(-c2ccc(N(c3cccc(-c4ccc5c(c4)oc4c(-c6ccccc6)cccc45)c3)c3ccc(-c4ccccc4)cc3-c3ccccc3)cc2)cc1 ; c1ccc(-c2ccc(N(c3cccc(-c4ccc5c(c4)oc4c(-c6ccccc6)cccc45)c3)c3ccc(-c4ccccc4)c(-c4ccccc4)c3)cc2)cc1 ; c1ccc(-c2ccc(-c3ccc(N(c4ccc(-c5ccccc5)cc4)c4cccc(-c5ccc6c(c5)oc5c(-c7ccccc7)cccc56)c4)cc3)cc2)cc1]

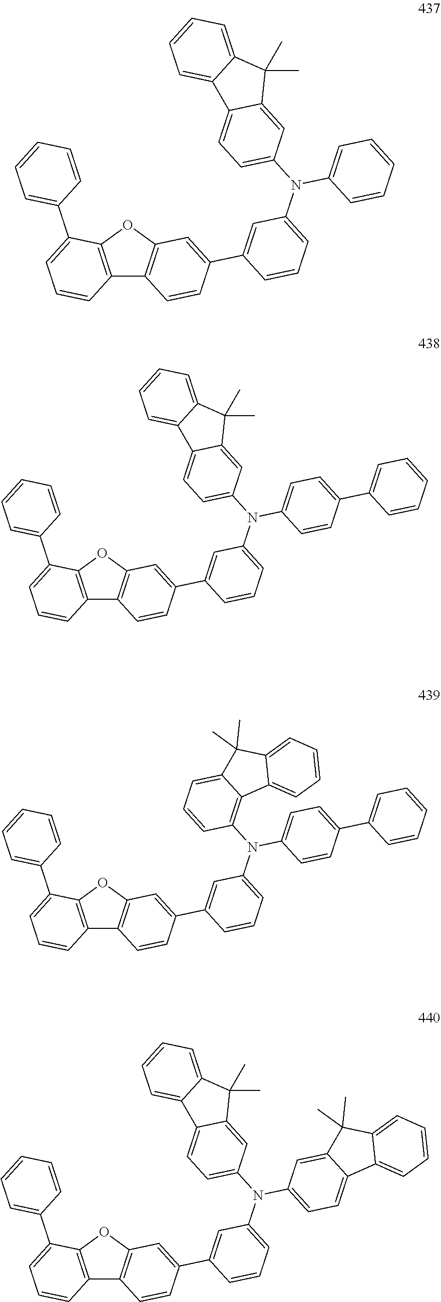 [SMILES: CC1(C)c2ccccc2-c2ccc(N(c3ccccc3)c3cccc(-c4ccc5c(c4)oc4c(-c6ccccc6)cccc45)c3)cc21 ; CC1(C)c2ccccc2-c2ccc(N(c3ccc(-c4ccccc4)cc3)c3cccc(-c4ccc5c(c4)oc4c(-c6ccccc6)cccc45)c3)cc21 ; CC1(C)c2ccccc2-c2c(N(c3ccc(-c4ccccc4)cc3)c3cccc(-c4ccc5c(c4)oc4c(-c6ccccc6)cccc45)c3)cccc21 ; CC1(C)c2ccccc2-c2ccc(N(c3cccc(-c4ccc5c(c4)oc4c(-c6ccccc6)cccc45)c3)c3ccc4c(c3)C(C)(C)c3ccccc3-4)cc21]

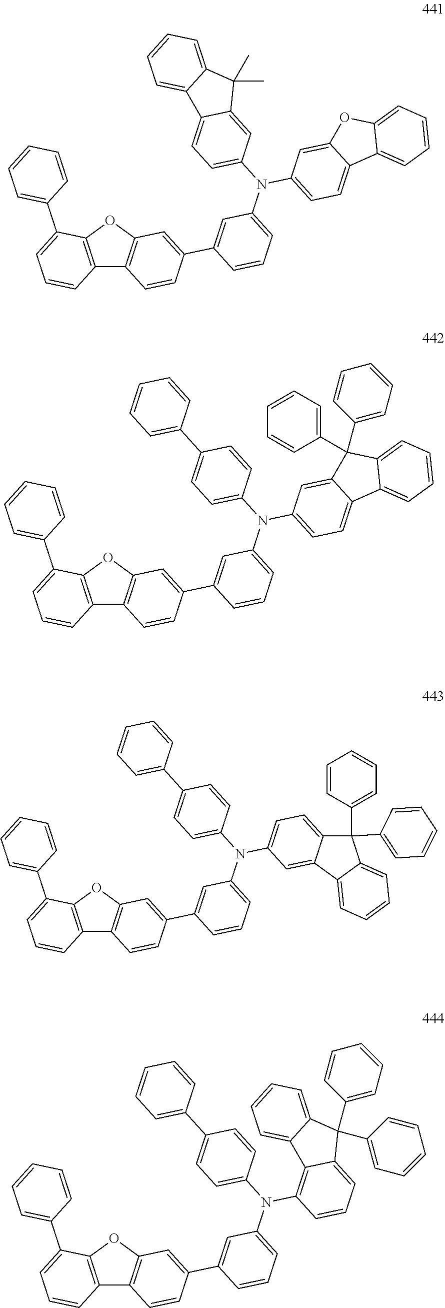 [SMILES: CC1(C)c2ccccc2-c2ccc(N(c3cccc(-c4ccc5c(c4)oc4c(-c6ccccc6)cccc45)c3)c3ccc4c(c3)oc3ccccc34)cc21 ; c1ccc(-c2ccc(N(c3cccc(-c4ccc5c(c4)oc4c(-c6ccccc6)cccc45)c3)c3ccc4c(c3)C(c3ccccc3)(c3ccccc3)c3ccccc3-4)cc2)cc1 ; c1ccc(-c2ccc(N(c3cccc(-c4ccc5c(c4)oc4c(-c6ccccc6)cccc45)c3)c3ccc4c(c3)-c3ccccc3C4(c3ccccc3)c3ccccc3)cc2)cc1 ; c1ccc(-c2ccc(N(c3cccc(-c4ccc5c(c4)oc4c(-c6ccccc6)cccc45)c3)c3cccc4c3-c3ccccc3C4(c3ccccc3)c3ccccc3)cc2)cc1]

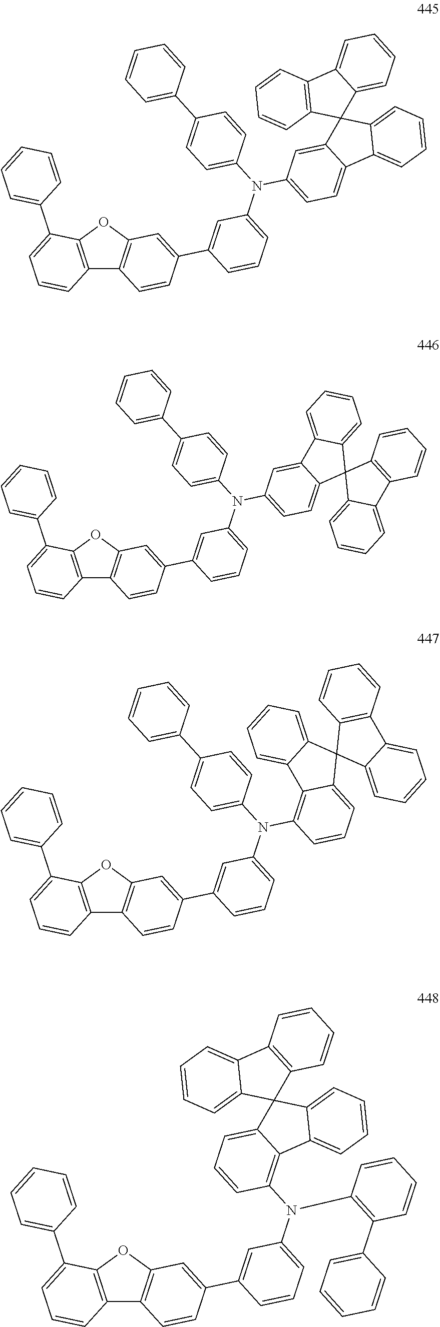 [SMILES: c1ccc(-c2ccc(N(c3cccc(-c4ccc5c(c4)oc4c(-c6ccccc6)cccc45)c3)c3ccc4c(c3)C3(c5ccccc5-c5ccccc53)c3ccccc3-4)cc2)cc1 ; c1ccc(-c2ccc(N(c3cccc(-c4ccc5c(c4)oc4c(-c6ccccc6)cccc45)c3)c3ccc4c(c3)-c3ccccc3C43c4ccccc4-c4ccccc43)cc2)cc1 ; c1ccc(-c2ccc(N(c3cccc(-c4ccc5c(c4)oc4c(-c6ccccc6)cccc45)c3)c3cccc4c3-c3ccccc3C43c4ccccc4-c4ccccc43)cc2)cc1 ; c1ccc(-c2ccccc2N(c2cccc(-c3ccc4c(c3)oc3c(-c5ccccc5)cccc34)c2)c2cccc3c2-c2ccccc2C32c3ccccc3-c3ccccc32)cc1]

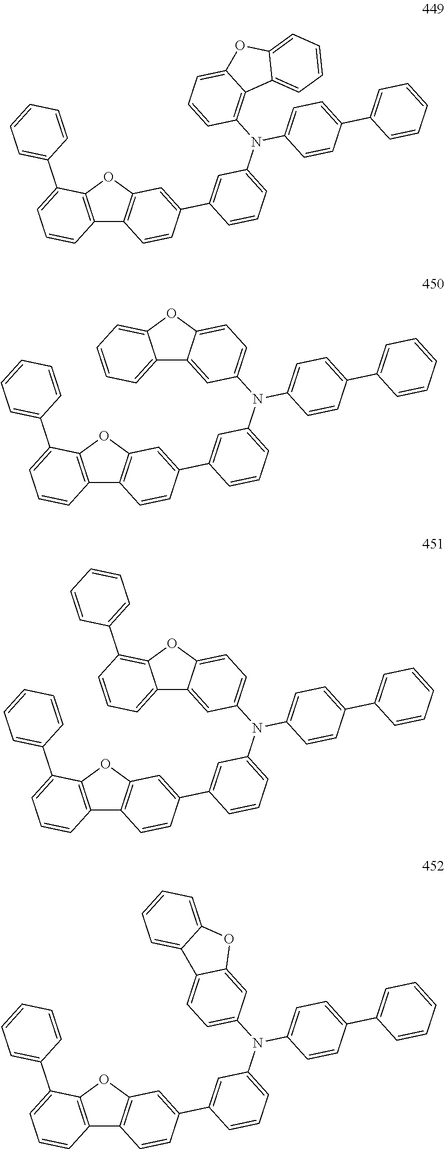 [SMILES: c1ccc(-c2ccc(N(c3cccc(-c4ccc5c(c4)oc4c(-c6ccccc6)cccc45)c3)c3cccc4oc5ccccc5c34)cc2)cc1 ; c1ccc(-c2ccc(N(c3cccc(-c4ccc5c(c4)oc4c(-c6ccccc6)cccc45)c3)c3ccc4oc5ccccc5c4c3)cc2)cc1 ; c1ccc(-c2ccc(N(c3cccc(-c4ccc5c(c4)oc4c(-c6ccccc6)cccc45)c3)c3ccc4oc5c(-c6ccccc6)cccc5c4c3)cc2)cc1 ; c1ccc(-c2ccc(N(c3cccc(-c4ccc5c(c4)oc4c(-c6ccccc6)cccc45)c3)c3ccc4c(c3)oc3ccccc34)cc2)cc1]

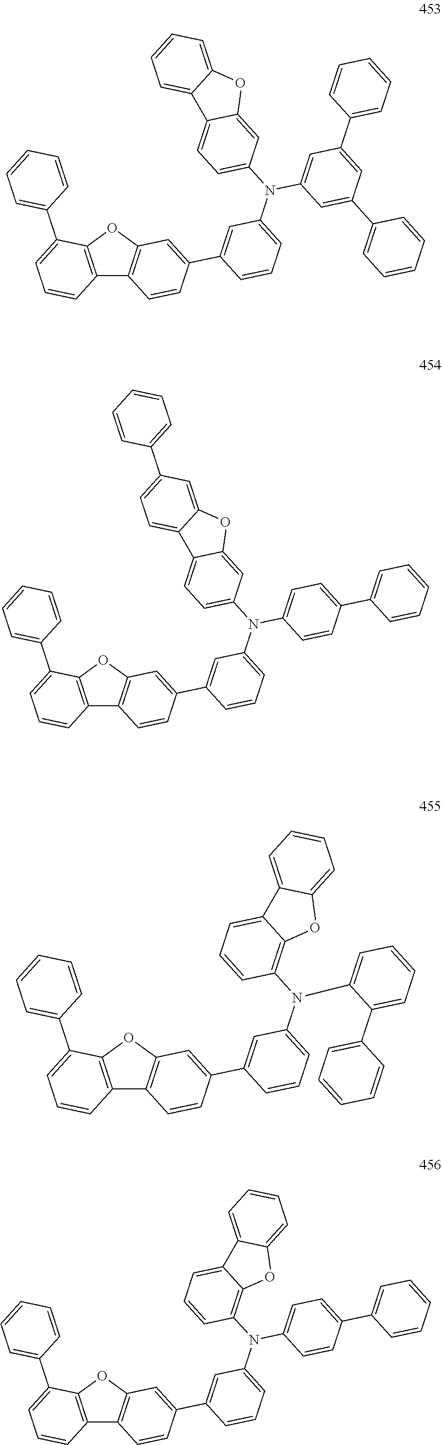 [SMILES: c1ccc(-c2cc(-c3ccccc3)cc(N(c3cccc(-c4ccc5c(c4)oc4c(-c6ccccc6)cccc45)c3)c3ccc4c(c3)oc3ccccc34)c2)cc1 ; c1ccc(-c2ccc(N(c3cccc(-c4ccc5c(c4)oc4c(-c6ccccc6)cccc45)c3)c3ccc4c(c3)oc3cc(-c5ccccc5)ccc34)cc2)cc1 ; c1ccc(-c2ccccc2N(c2cccc(-c3ccc4c(c3)oc3c(-c5ccccc5)cccc34)c2)c2cccc3c2oc2ccccc23)cc1 ; c1ccc(-c2ccc(N(c3cccc(-c4ccc5c(c4)oc4c(-c6ccccc6)cccc45)c3)c3cccc4c3oc3ccccc34)cc2)cc1]

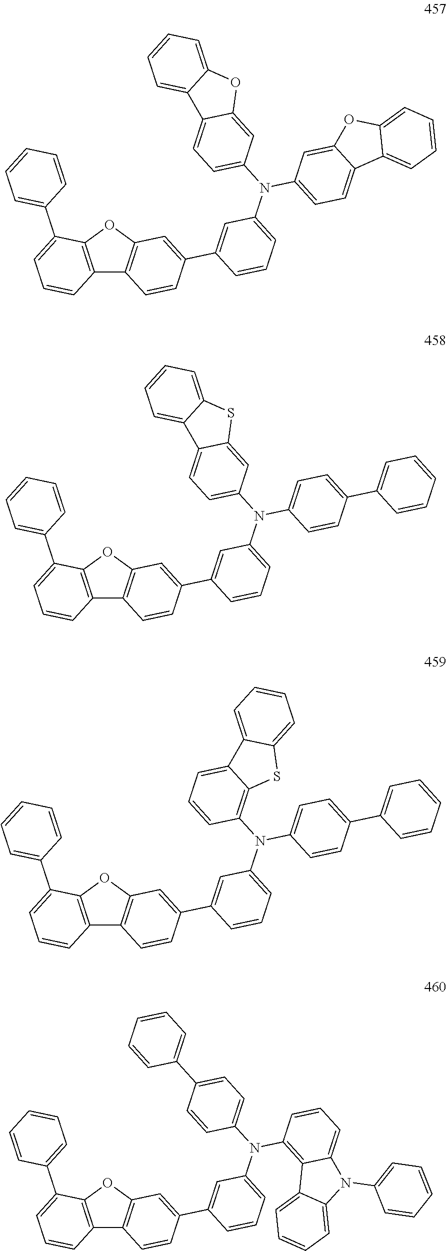 [SMILES: c1ccc(-c2cccc3c2oc2cc(-c4cccc(N(c5ccc6c(c5)oc5ccccc56)c5ccc6c(c5)oc5ccccc56)c4)ccc23)cc1 ; c1ccc(-c2ccc(N(c3cccc(-c4ccc5c(c4)oc4c(-c6ccccc6)cccc45)c3)c3ccc4c(c3)sc3ccccc34)cc2)cc1 ; c1ccc(-c2ccc(N(c3cccc(-c4ccc5c(c4)oc4c(-c6ccccc6)cccc45)c3)c3cccc4c3sc3ccccc34)cc2)cc1 ; c1ccc(-c2ccc(N(c3cccc(-c4ccc5c(c4)oc4c(-c6ccccc6)cccc45)c3)c3cccc4c3c3ccccc3n4-c3ccccc3)cc2)cc1]

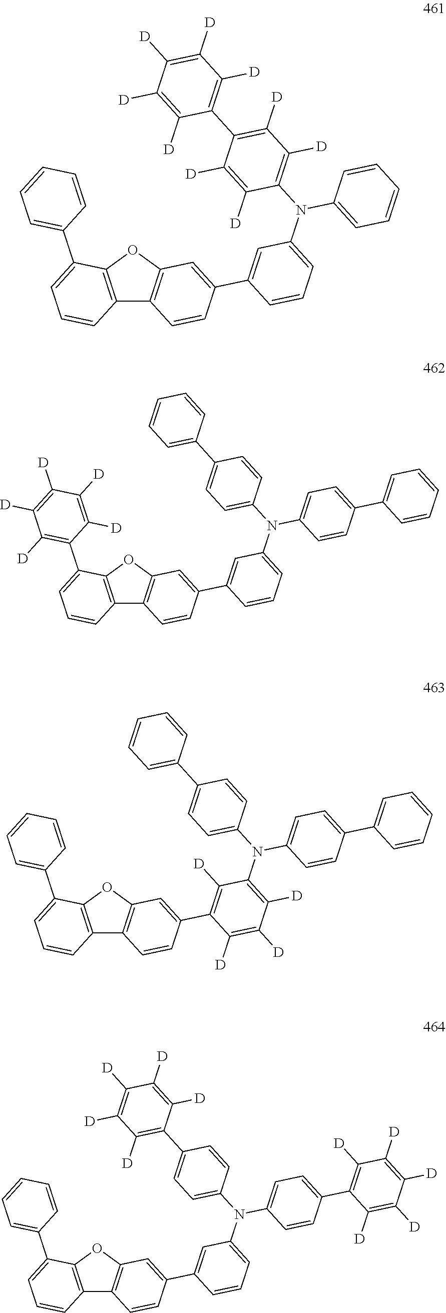 [SMILES: [2H]c1c([2H])c([2H])c(-c2c([2H])c([2H])c(N(c3ccccc3)c3cccc(-c4ccc5c(c4)oc4c(-c6ccccc6)cccc45)c3)c([2H])c2[2H])c([2H])c1[2H] ; [2H]c1c([2H])c([2H])c(-c2cccc3c2oc2cc(-c4cccc(N(c5ccc(-c6ccccc6)cc5)c5ccc(-c6ccccc6)cc5)c4)ccc23)c([2H])c1[2H] ; [2H]c1c([2H])c(-c2ccc3c(c2)oc2c(-c4ccccc4)cccc23)c([2H])c(N(c2ccc(-c3ccccc3)cc2)c2ccc(-c3ccccc3)cc2)c1[2H] ; [2H]c1c([2H])c([2H])c(-c2ccc(N(c3ccc(-c4c([2H])c([2H])c([2H])c([2H])c4[2H])cc3)c3cccc(-c4ccc5c(c4)oc4c(-c6ccccc6)cccc45)c3)cc2)c([2H])c1[2H]]

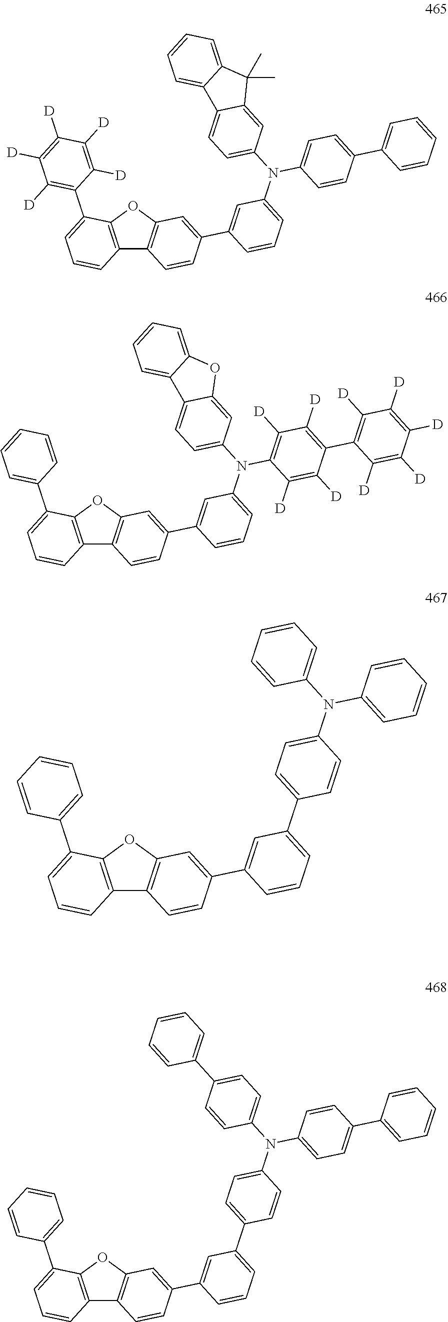 [SMILES: [2H]c1c([2H])c([2H])c(-c2cccc3c2oc2cc(-c4cccc(N(c5ccc(-c6ccccc6)cc5)c5ccc6c(c5)C(C)(C)c5ccccc5-6)c4)ccc23)c([2H])c1[2H] ; [2H]c1c([2H])c([2H])c(-c2c([2H])c([2H])c(N(c3cccc(-c4ccc5c(c4)oc4c(-c6ccccc6)cccc45)c3)c3ccc4c(c3)oc3ccccc34)c([2H])c2[2H])c([2H])c1[2H] ; c1ccc(-c2cccc3c2oc2cc(-c4cccc(-c5ccc(N(c6ccccc6)c6ccccc6)cc5)c4)ccc23)cc1 ; c1ccc(-c2ccc(N(c3ccc(-c4ccccc4)cc3)c3ccc(-c4cccc(-c5ccc6c(c5)oc5c(-c7ccccc7)cccc56)c4)cc3)cc2)cc1]

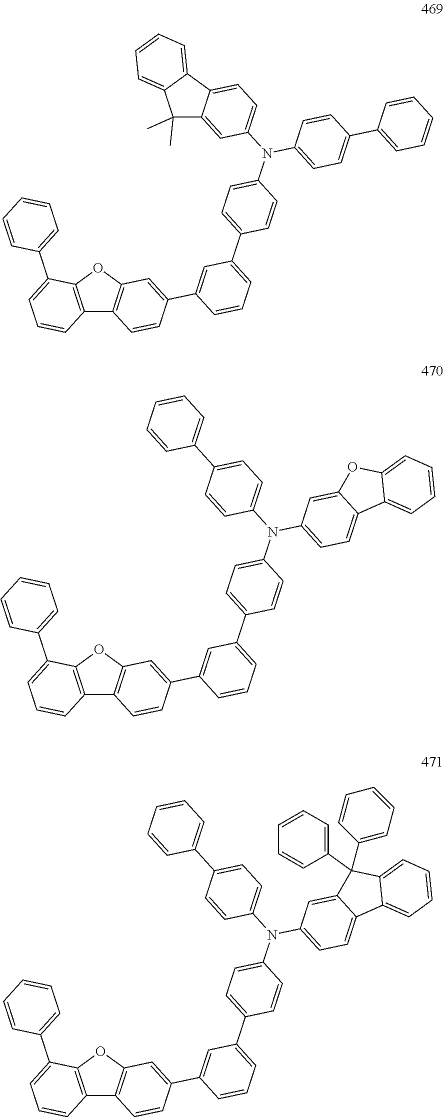 [SMILES: CC1(C)c2ccccc2-c2ccc(N(c3ccc(-c4ccccc4)cc3)c3ccc(-c4cccc(-c5ccc6c(c5)oc5c(-c7ccccc7)cccc56)c4)cc3)cc21 ; c1ccc(-c2ccc(N(c3ccc(-c4cccc(-c5ccc6c(c5)oc5c(-c7ccccc7)cccc56)c4)cc3)c3ccc4c(c3)oc3ccccc34)cc2)cc1 ; c1ccc(-c2ccc(N(c3ccc(-c4cccc(-c5ccc6c(c5)oc5c(-c7ccccc7)cccc56)c4)cc3)c3ccc4c(c3)C(c3ccccc3)(c3ccccc3)c3ccccc3-4)cc2)cc1]

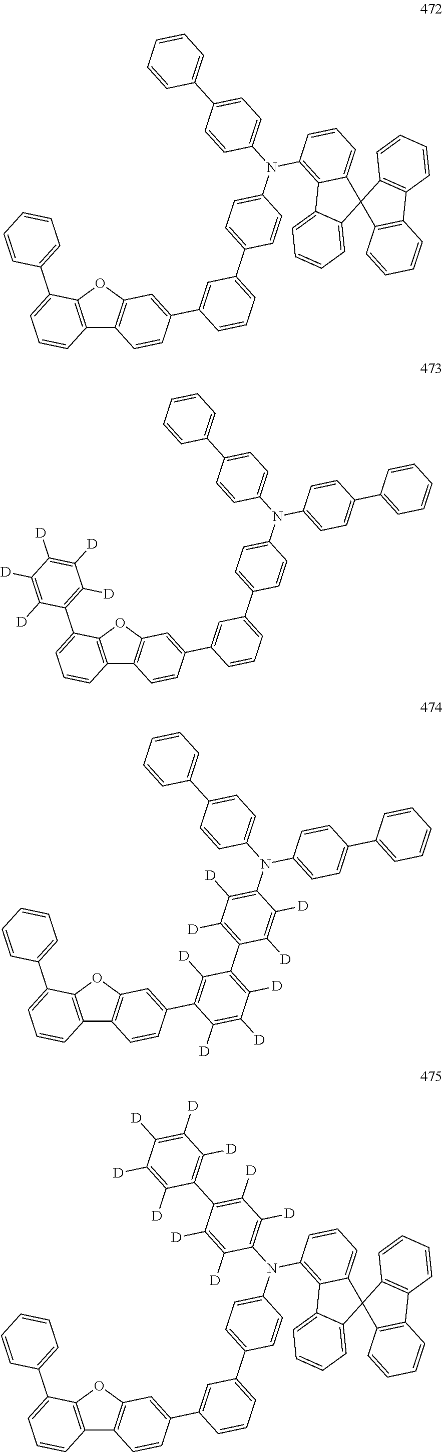

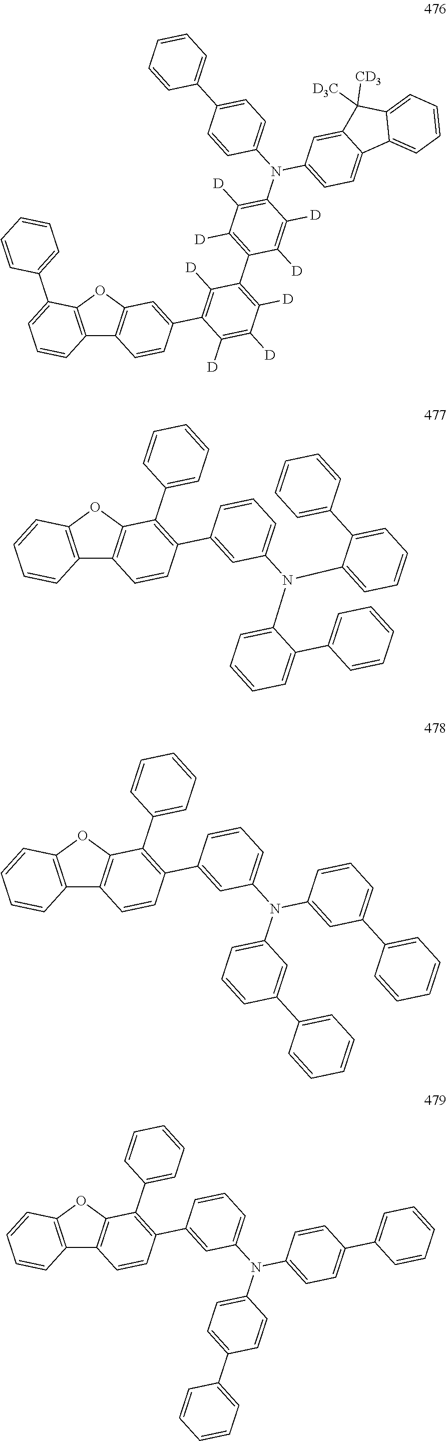 [SMILES: [2H]c1c([2H])c(-c2ccc3c(c2)oc2c(-c4ccccc4)cccc23)c([2H])c(-c2c([2H])c([2H])c(N(c3ccc(-c4ccccc4)cc3)c3ccc4c(c3)C(C([2H])([2H])[2H])(C([2H])([2H])[2H])c3ccccc3-4)c([2H])c2[2H])c1[2H] ; c1ccc(-c2ccccc2N(c2cccc(-c3ccc4c(oc5ccccc54)c3-c3ccccc3)c2)c2ccccc2-c2ccccc2)cc1 ; c1ccc(-c2cccc(N(c3cccc(-c4ccccc4)c3)c3cccc(-c4ccc5c(oc6ccccc65)c4-c4ccccc4)c3)c2)cc1 ; c1ccc(-c2ccc(N(c3ccc(-c4ccccc4)cc3)c3cccc(-c4ccc5c(oc6ccccc65)c4-c4ccccc4)c3)cc2)cc1]

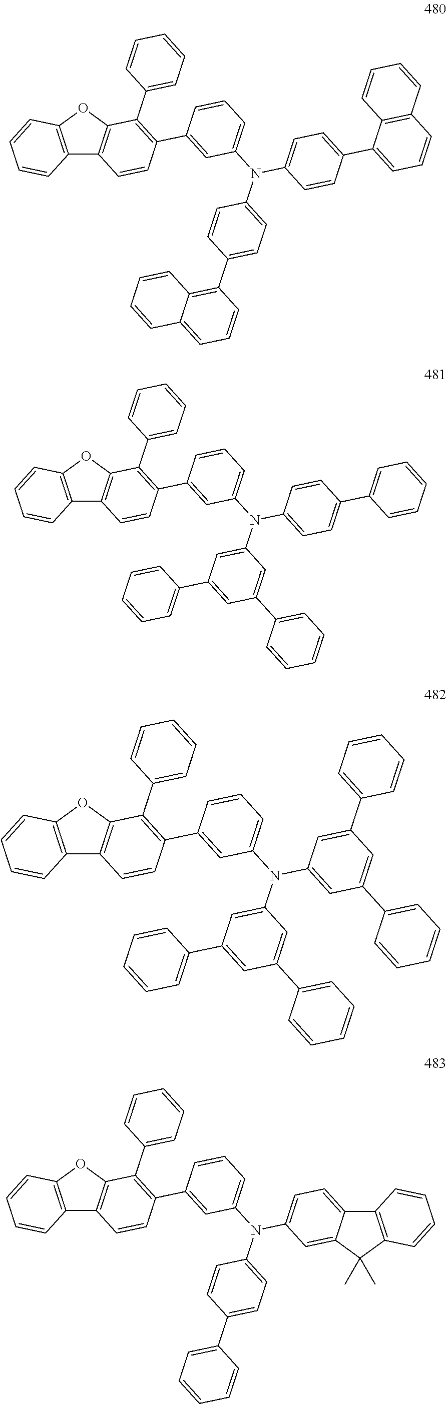 [SMILES: c1ccc(-c2c(-c3cccc(N(c4ccc(-c5cccc6ccccc56)cc4)c4ccc(-c5cccc6ccccc56)cc4)c3)ccc3c2oc2ccccc23)cc1 ; c1ccc(-c2ccc(N(c3cc(-c4ccccc4)cc(-c4ccccc4)c3)c3cccc(-c4ccc5c(oc6ccccc65)c4-c4ccccc4)c3)cc2)cc1 ; c1ccc(-c2cc(-c3ccccc3)cc(N(c3cc(-c4ccccc4)cc(-c4ccccc4)c3)c3cccc(-c4ccc5c(oc6ccccc65)c4-c4ccccc4)c3)c2)cc1 ; CC1(C)c2ccccc2-c2ccc(N(c3ccc(-c4ccccc4)cc3)c3cccc(-c4ccc5c(oc6ccccc65)c4-c4ccccc4)c3)cc21]

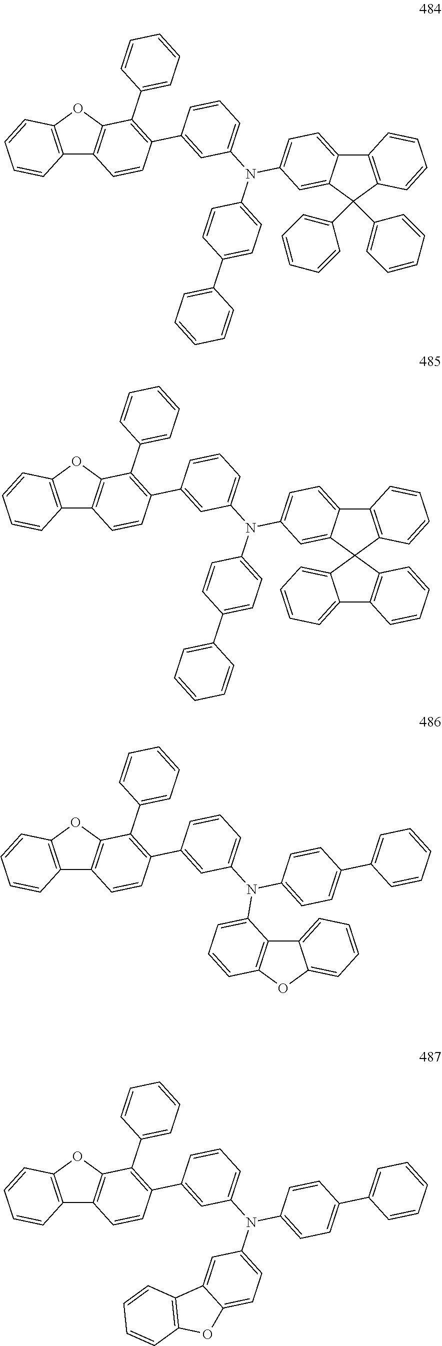 [SMILES: c1ccc(-c2ccc(N(c3cccc(-c4ccc5c(oc6ccccc65)c4-c4ccccc4)c3)c3ccc4c(c3)C(c3ccccc3)(c3ccccc3)c3ccccc3-4)cc2)cc1 ; c1ccc(-c2ccc(N(c3cccc(-c4ccc5c(oc6ccccc65)c4-c4ccccc4)c3)c3ccc4c(c3)C3(c5ccccc5-c5ccccc53)c3ccccc3-4)cc2)cc1 ; c1ccc(-c2ccc(N(c3cccc(-c4ccc5c(oc6ccccc65)c4-c4ccccc4)c3)c3cccc4oc5ccccc5c34)cc2)cc1 ; c1ccc(-c2ccc(N(c3cccc(-c4ccc5c(oc6ccccc65)c4-c4ccccc4)c3)c3ccc4oc5ccccc5c4c3)cc2)cc1]

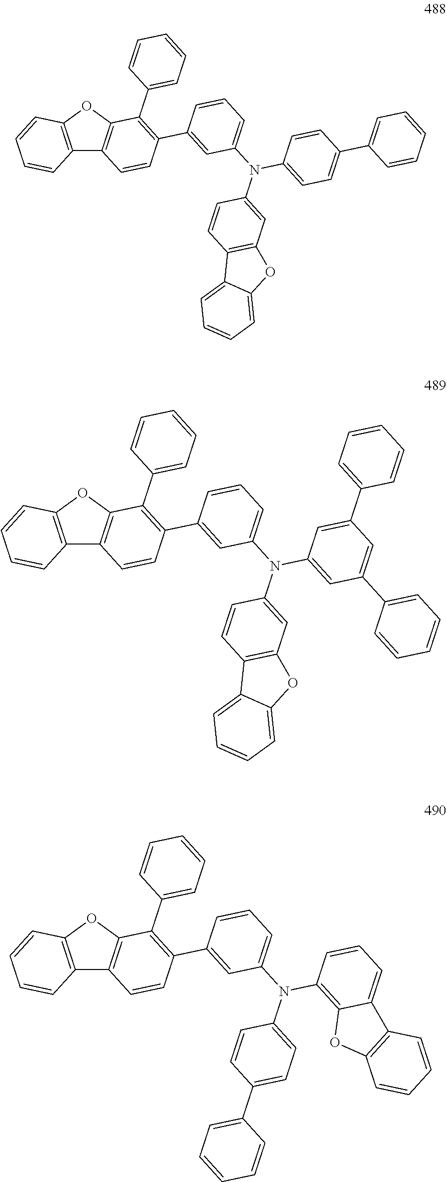 [SMILES: c1ccc(-c2ccc(N(c3cccc(-c4ccc5c(oc6ccccc65)c4-c4ccccc4)c3)c3ccc4c(c3)oc3ccccc34)cc2)cc1 ; c1ccc(-c2cc(-c3ccccc3)cc(N(c3cccc(-c4ccc5c(oc6ccccc65)c4-c4ccccc4)c3)c3ccc4c(c3)oc3ccccc34)c2)cc1 ; c1ccc(-c2ccc(N(c3cccc(-c4ccc5c(oc6ccccc65)c4-c4ccccc4)c3)c3cccc4c3oc3ccccc34)cc2)cc1]

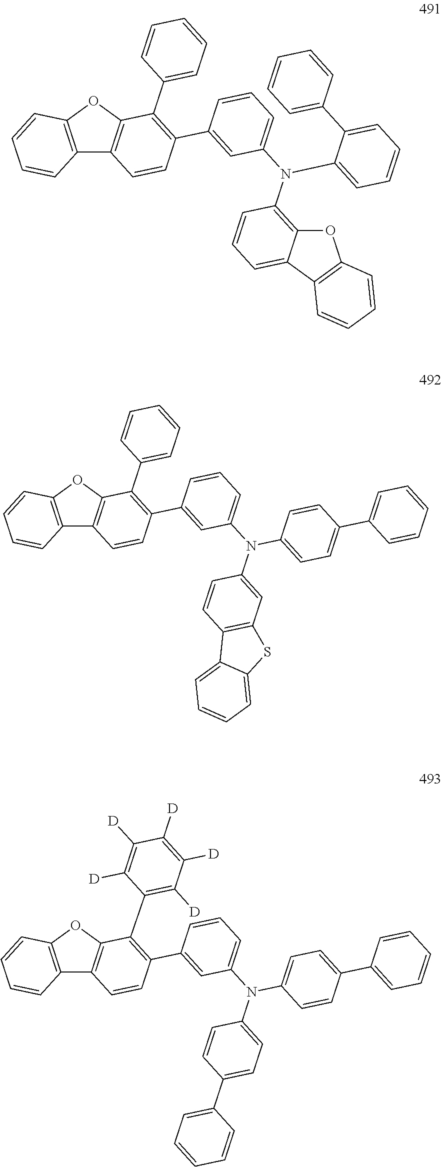 [SMILES: c1ccc(-c2ccccc2N(c2cccc(-c3ccc4c(oc5ccccc54)c3-c3ccccc3)c2)c2cccc3c2oc2ccccc23)cc1 ; c1ccc(-c2ccc(N(c3cccc(-c4ccc5c(oc6ccccc65)c4-c4ccccc4)c3)c3ccc4c(c3)sc3ccccc34)cc2)cc1 ; [2H]c1c([2H])c([2H])c(-c2c(-c3cccc(N(c4ccc(-c5ccccc5)cc4)c4ccc(-c5ccccc5)cc4)c3)ccc3c2oc2ccccc23)c([2H])c1[2H]]

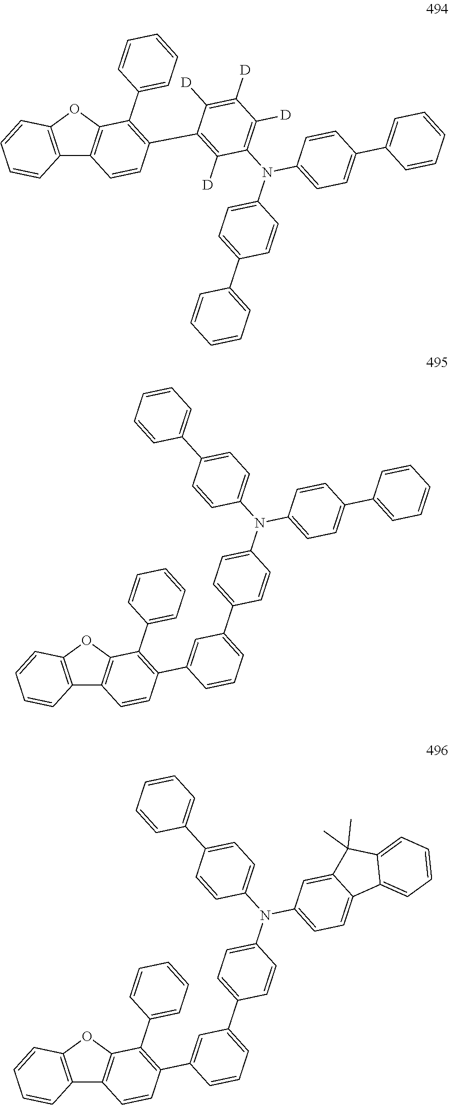 [SMILES: [2H]c1c([2H])c(-c2ccc3c(oc4ccccc43)c2-c2ccccc2)c([2H])c(N(c2ccc(-c3ccccc3)cc2)c2ccc(-c3ccccc3)cc2)c1[2H] ; c1ccc(-c2ccc(N(c3ccc(-c4ccccc4)cc3)c3ccc(-c4cccc(-c5ccc6c(oc7ccccc76)c5-c5ccccc5)c4)cc3)cc2)cc1 ; CC1(C)c2ccccc2-c2ccc(N(c3ccc(-c4ccccc4)cc3)c3ccc(-c4cccc(-c5ccc6c(oc7ccccc76)c5-c5ccccc5)c4)cc3)cc21]

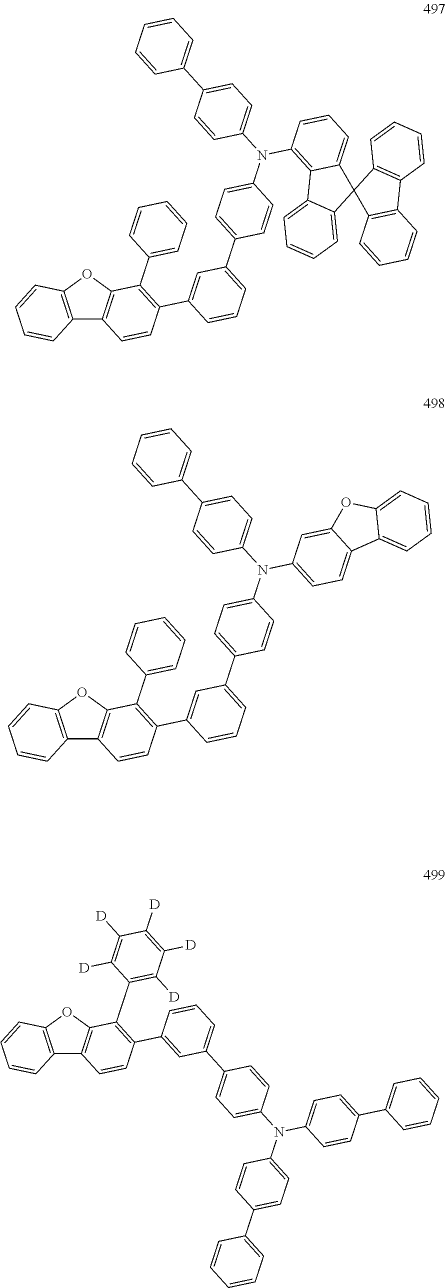 [SMILES: c1ccc(-c2ccc(N(c3ccc(-c4cccc(-c5ccc6c(oc7ccccc76)c5-c5ccccc5)c4)cc3)c3cccc4c3-c3ccccc3C43c4ccccc4-c4ccccc43)cc2)cc1 ; c1ccc(-c2ccc(N(c3ccc(-c4cccc(-c5ccc6c(oc7ccccc76)c5-c5ccccc5)c4)cc3)c3ccc4c(c3)oc3ccccc34)cc2)cc1 ; [2H]c1c([2H])c([2H])c(-c2c(-c3cccc(-c4ccc(N(c5ccc(-c6ccccc6)cc5)c5ccc(-c6ccccc6)cc5)cc4)c3)ccc3c2oc2ccccc23)c([2H])c1[2H]]

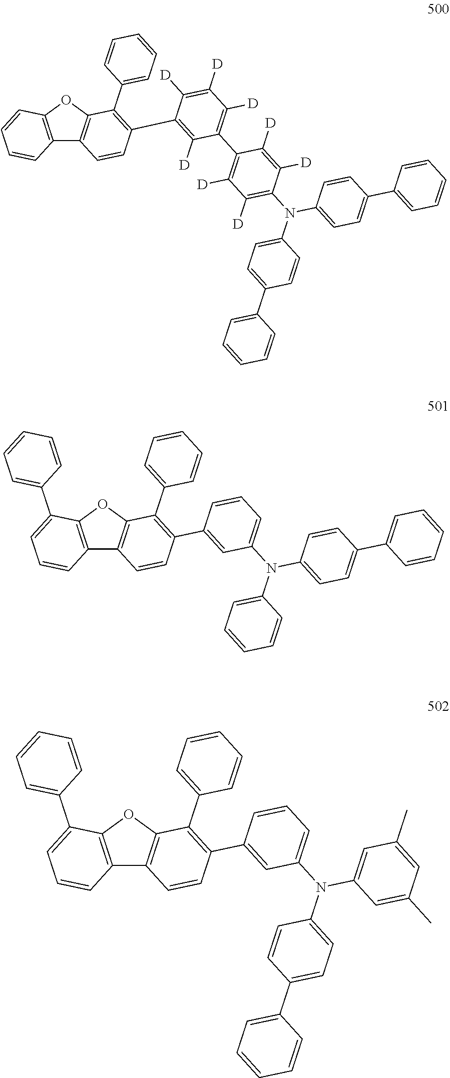 [SMILES: [2H]c1c([2H])c(-c2ccc3c(oc4ccccc43)c2-c2ccccc2)c([2H])c(-c2c([2H])c([2H])c(N(c3ccc(-c4ccccc4)cc3)c3ccc(-c4ccccc4)cc3)c([2H])c2[2H])c1[2H] ; c1ccc(-c2ccc(N(c3ccccc3)c3cccc(-c4ccc5c(oc6c(-c7ccccc7)cccc65)c4-c4ccccc4)c3)cc2)cc1 ; Cc1cc(C)cc(N(c2ccc(-c3ccccc3)cc2)c2cccc(-c3ccc4c(oc5c(-c6ccccc6)cccc54)c3-c3ccccc3)c2)c1]

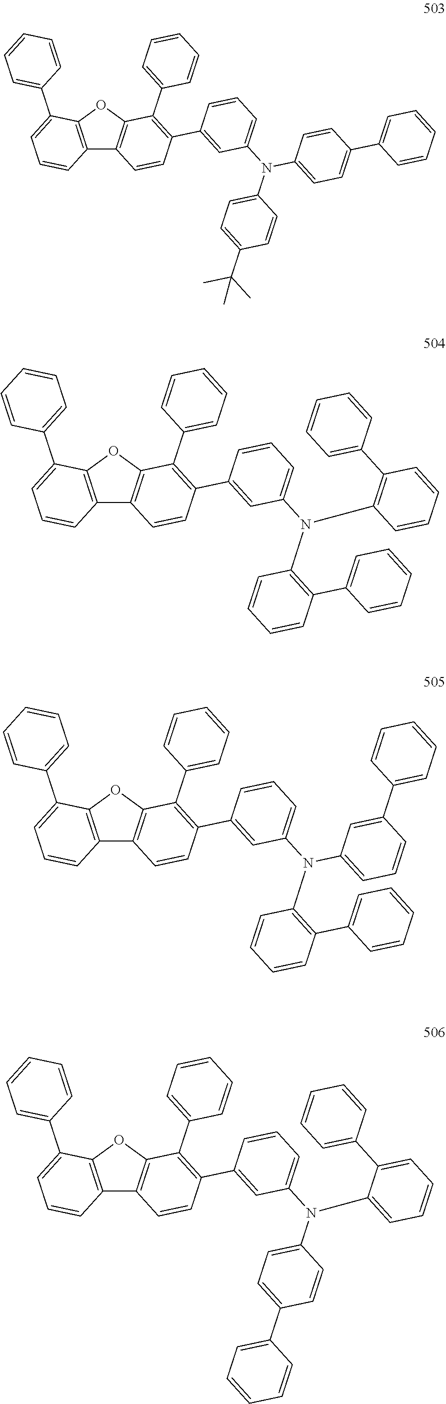 [SMILES: CC(C)(C)c1ccc(N(c2ccc(-c3ccccc3)cc2)c2cccc(-c3ccc4c(oc5c(-c6ccccc6)cccc54)c3-c3ccccc3)c2)cc1 ; c1ccc(-c2ccccc2N(c2cccc(-c3ccc4c(oc5c(-c6ccccc6)cccc54)c3-c3ccccc3)c2)c2ccccc2-c2ccccc2)cc1 ; c1ccc(-c2cccc(N(c3cccc(-c4ccc5c(oc6c(-c7ccccc7)cccc65)c4-c4ccccc4)c3)c3ccccc3-c3ccccc3)c2)cc1 ; c1ccc(-c2ccc(N(c3cccc(-c4ccc5c(oc6c(-c7ccccc7)cccc65)c4-c4ccccc4)c3)c3ccccc3-c3ccccc3)cc2)cc1]

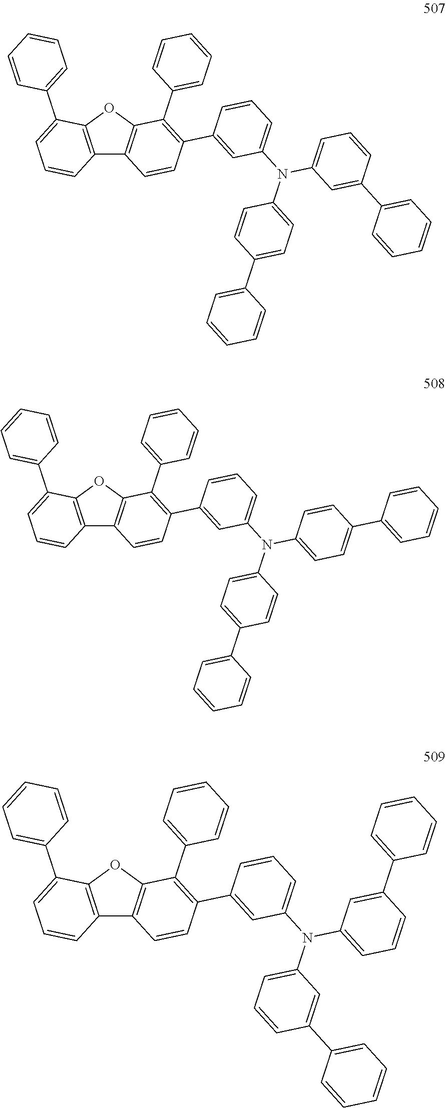 [SMILES: c1ccc(-c2ccc(N(c3cccc(-c4ccccc4)c3)c3cccc(-c4ccc5c(oc6c(-c7ccccc7)cccc65)c4-c4ccccc4)c3)cc2)cc1 ; c1ccc(-c2ccc(N(c3ccc(-c4ccccc4)cc3)c3cccc(-c4ccc5c(oc6c(-c7ccccc7)cccc65)c4-c4ccccc4)c3)cc2)cc1 ; c1ccc(-c2cccc(N(c3cccc(-c4ccccc4)c3)c3cccc(-c4ccc5c(oc6c(-c7ccccc7)cccc65)c4-c4ccccc4)c3)c2)cc1]

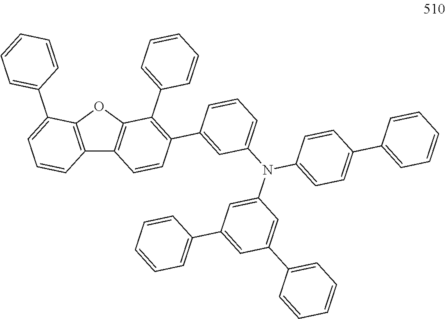 [SMILES: c1ccc(-c2ccc(N(c3cc(-c4ccccc4)cc(-c4ccccc4)c3)c3cccc(-c4ccc5c(oc6c(-c7ccccc7)cccc65)c4-c4ccccc4)c3)cc2)cc1]

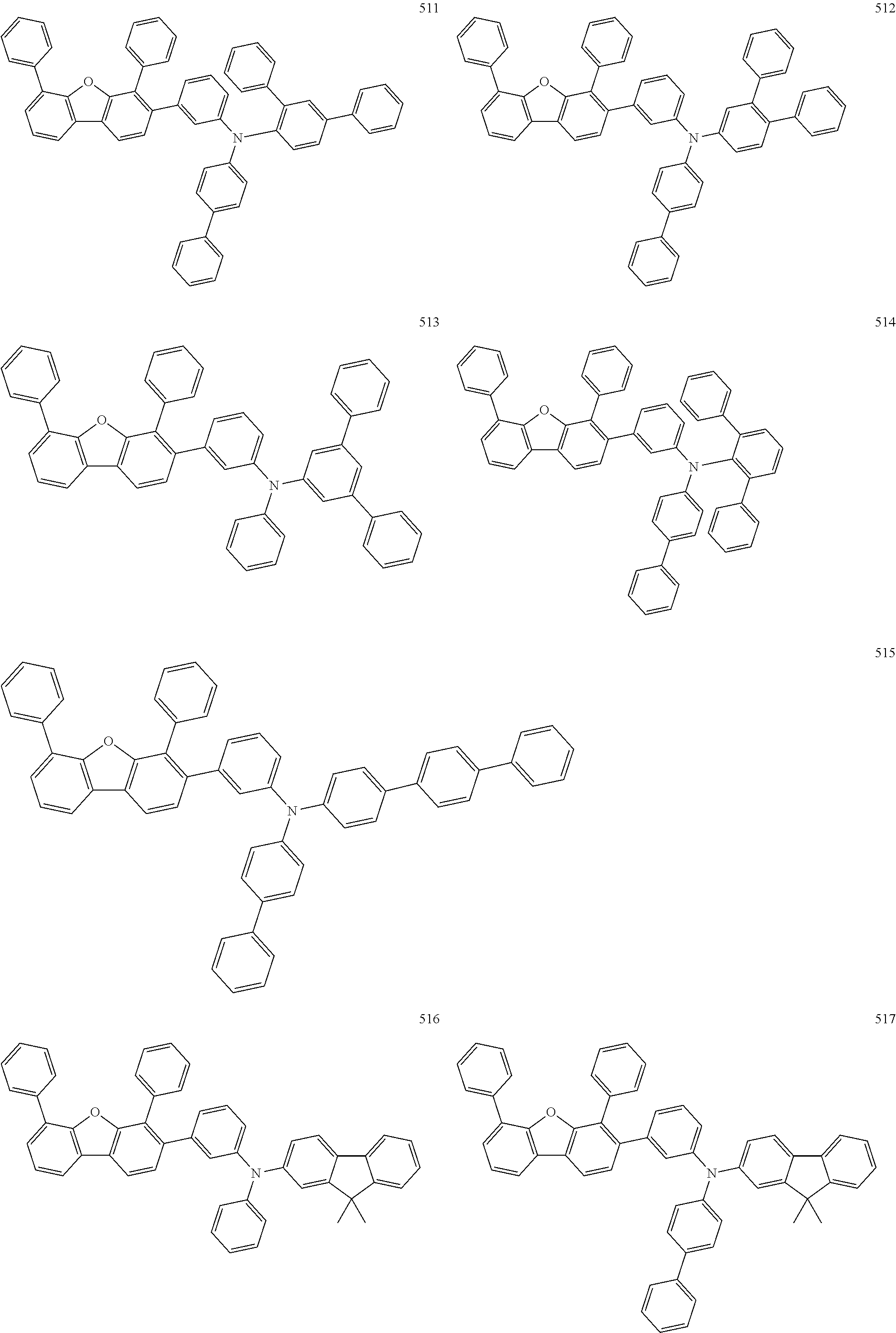

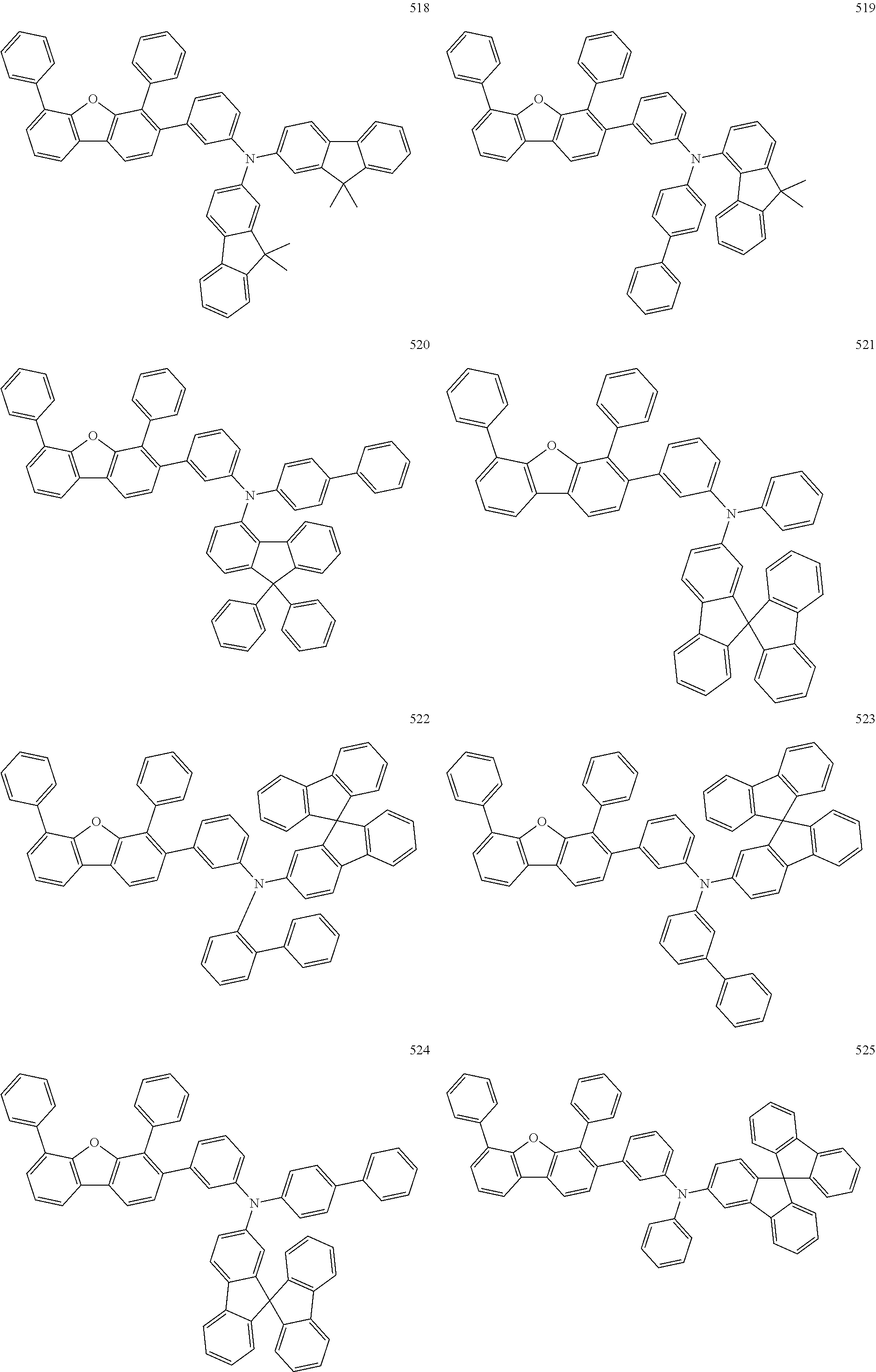

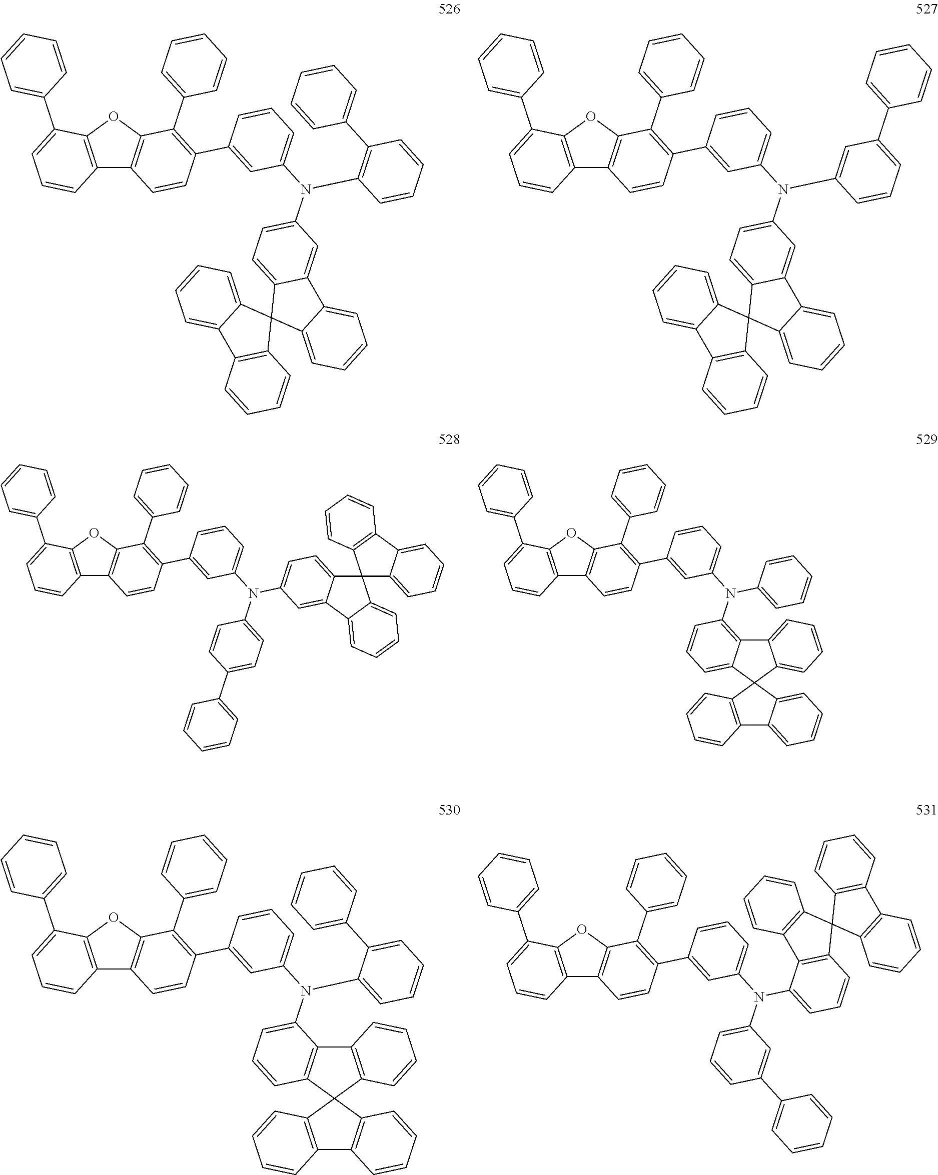

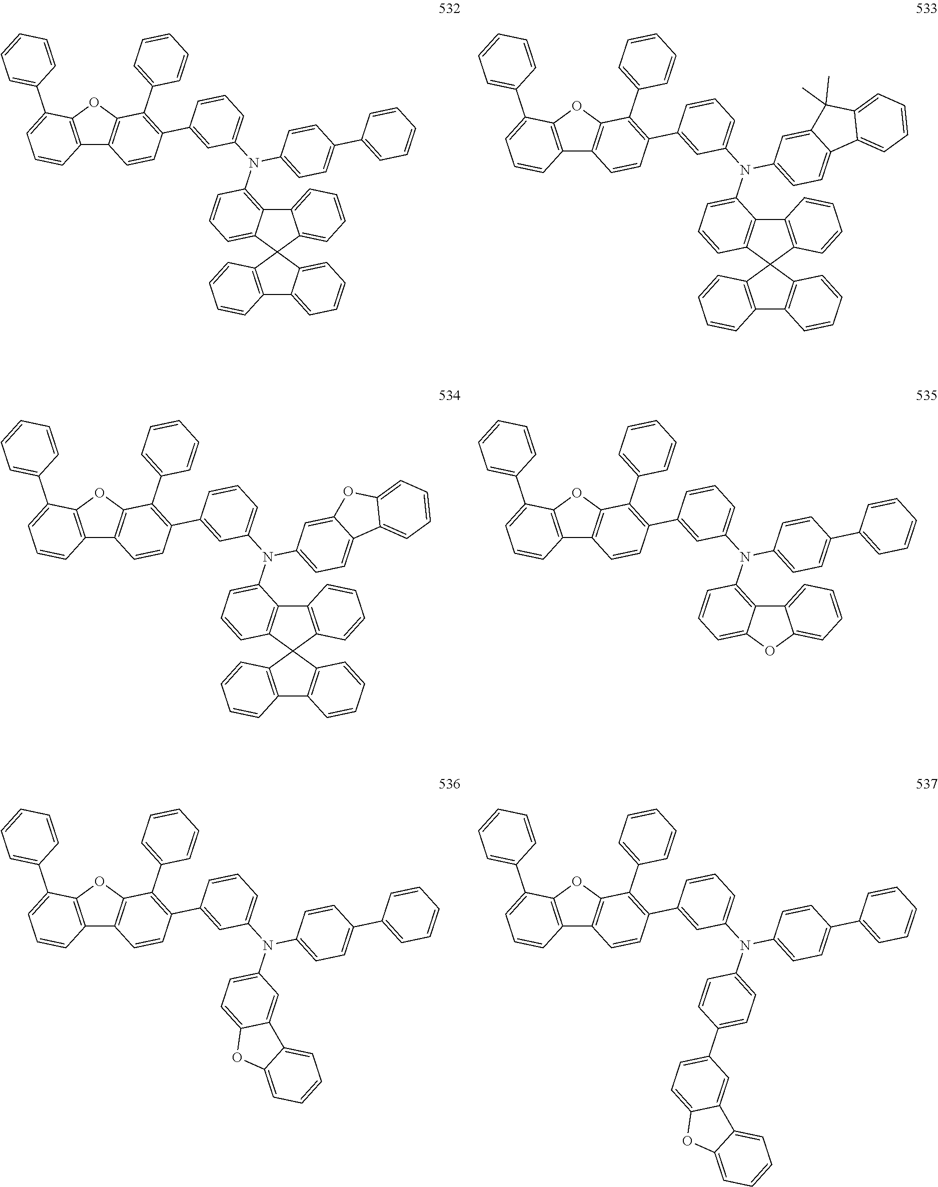

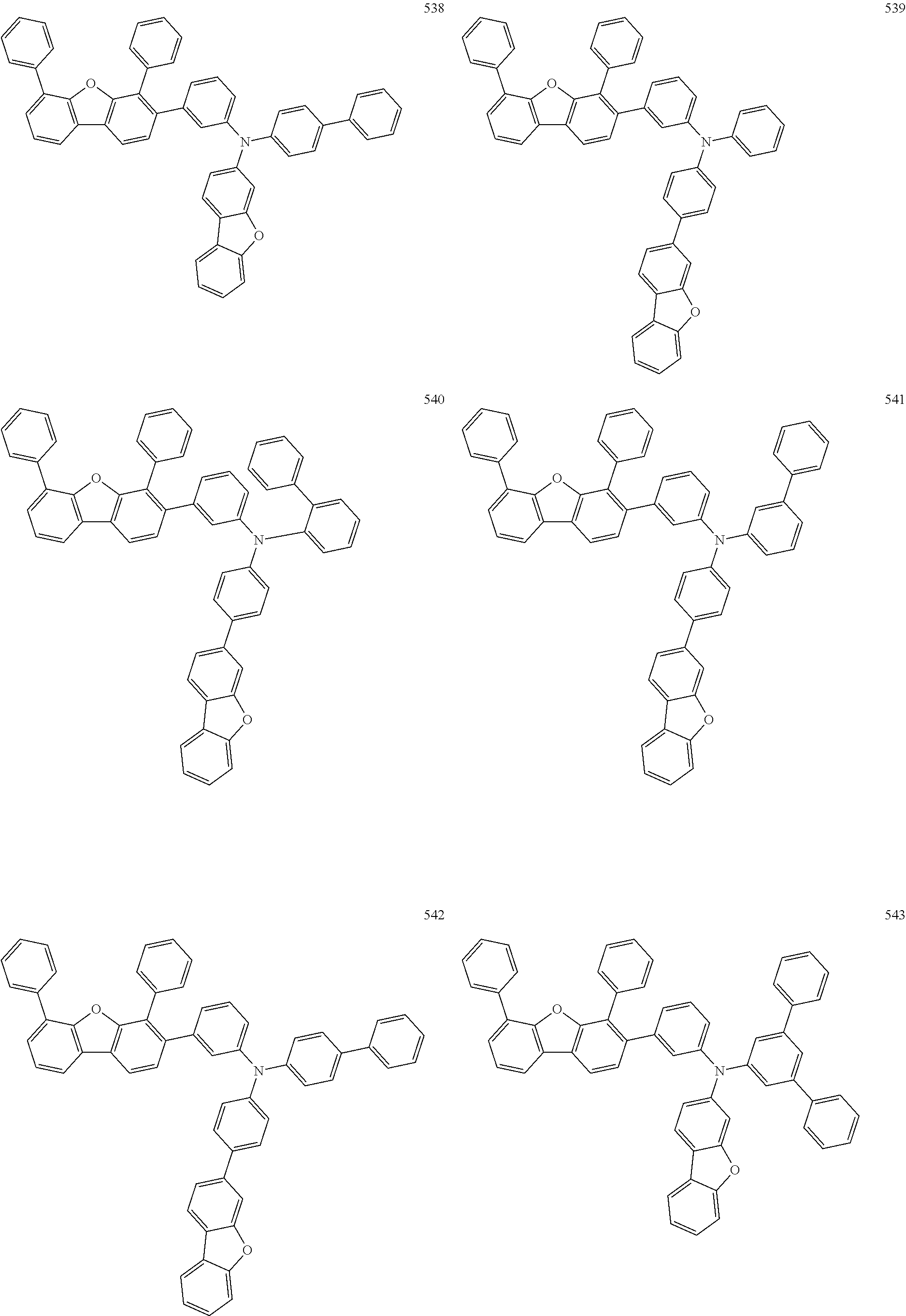

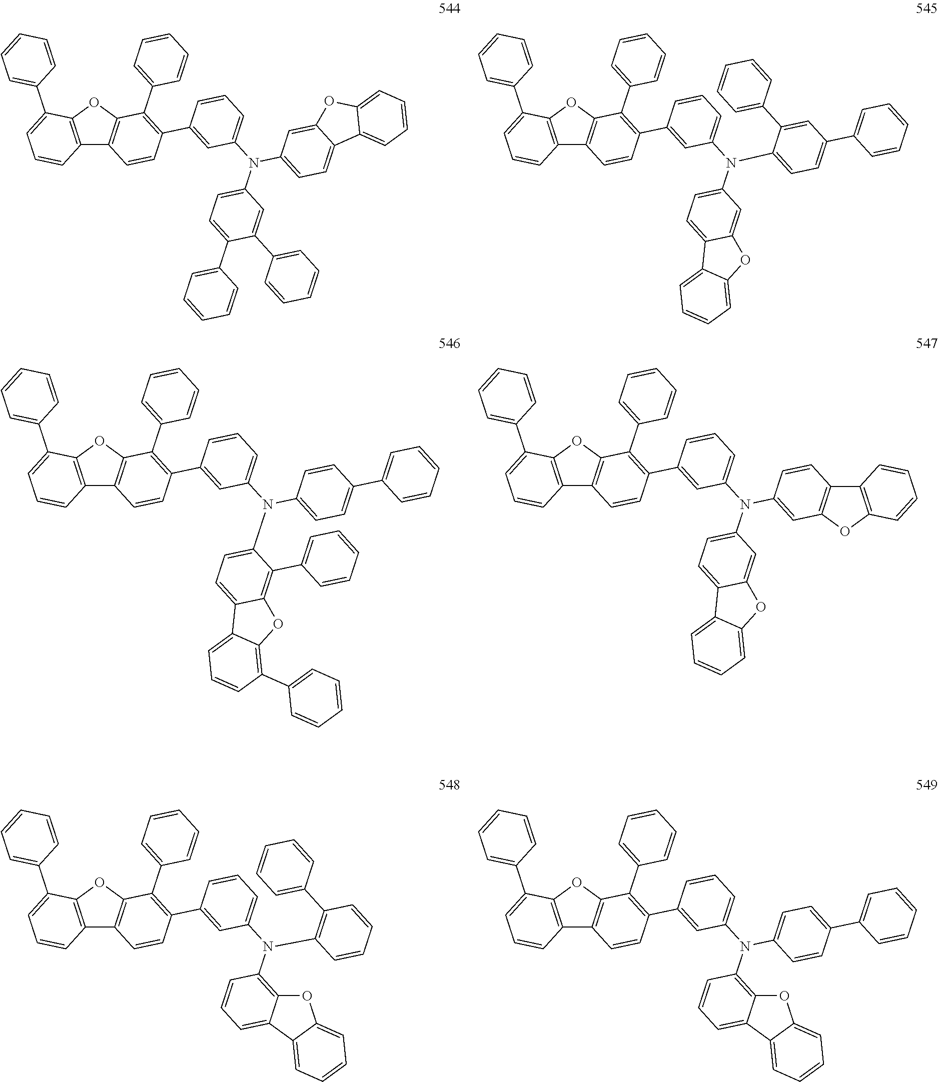

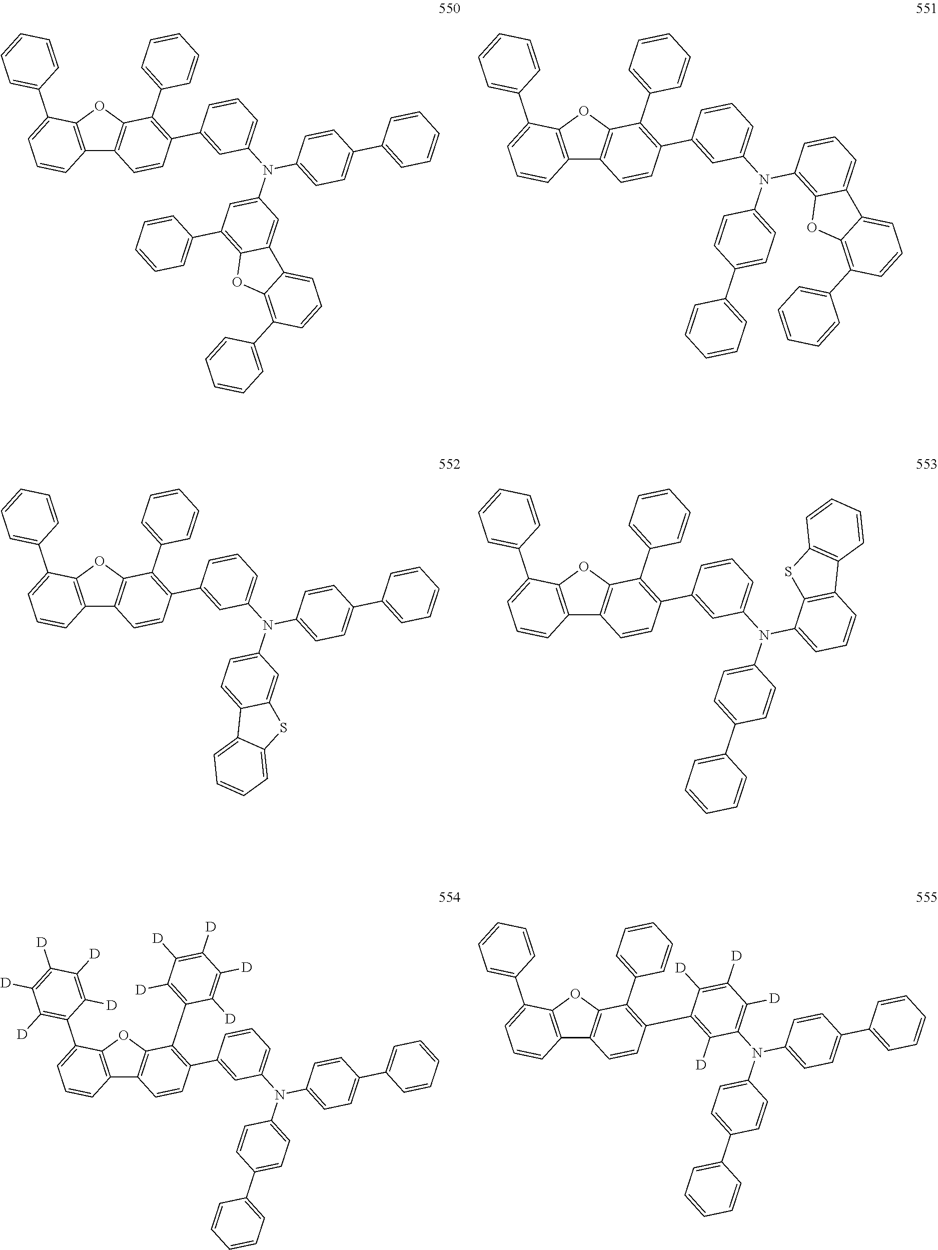

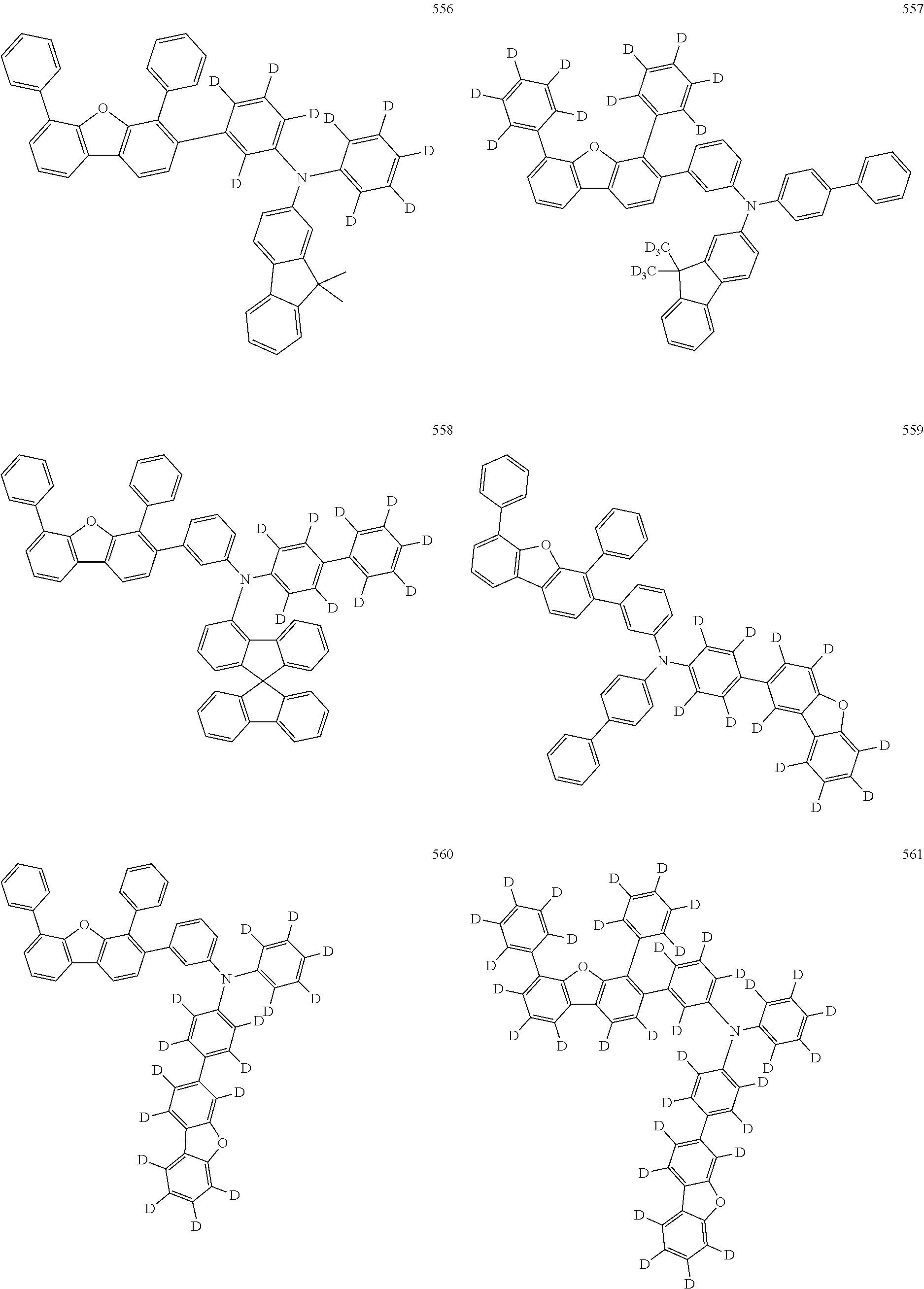

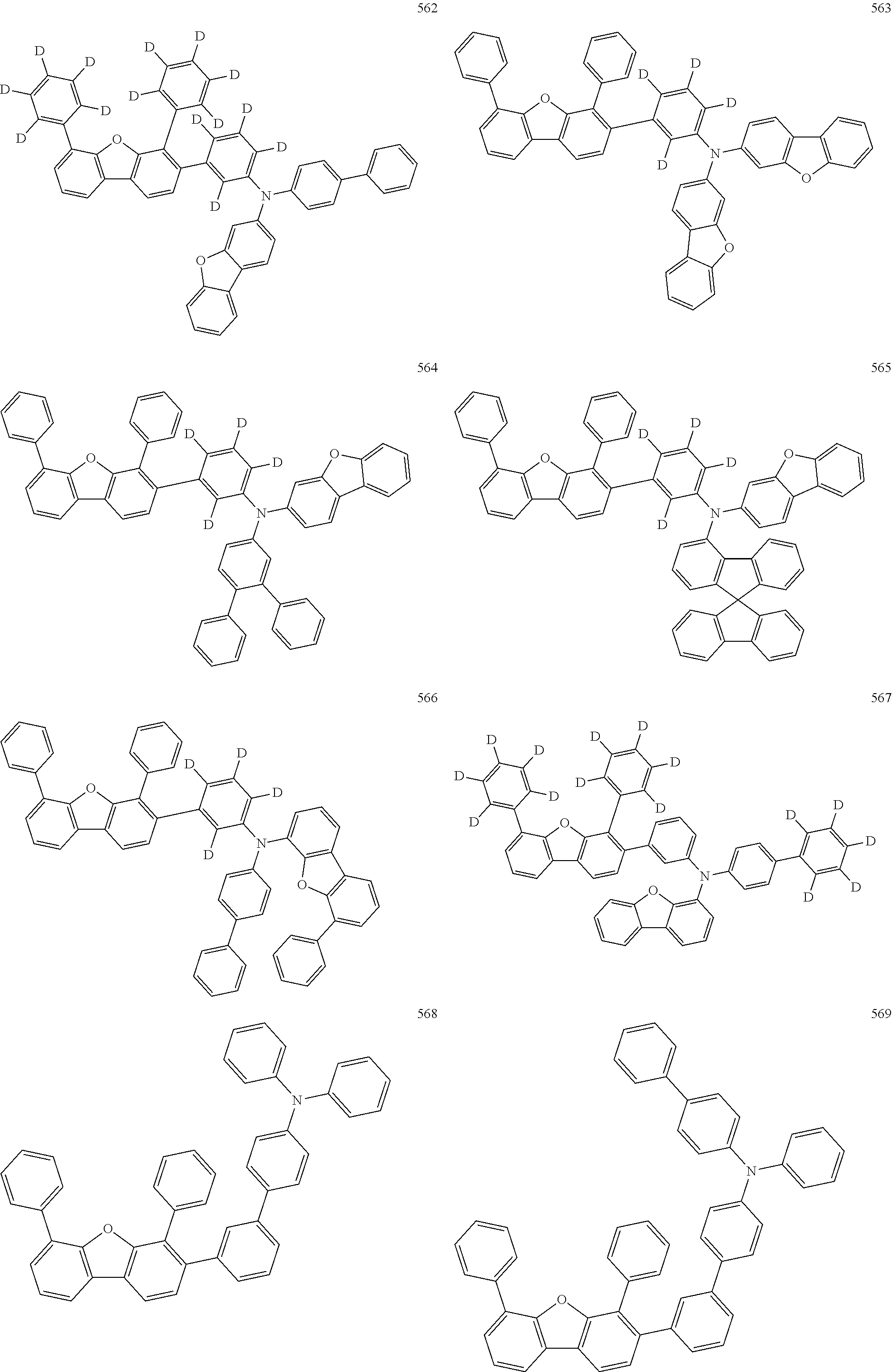

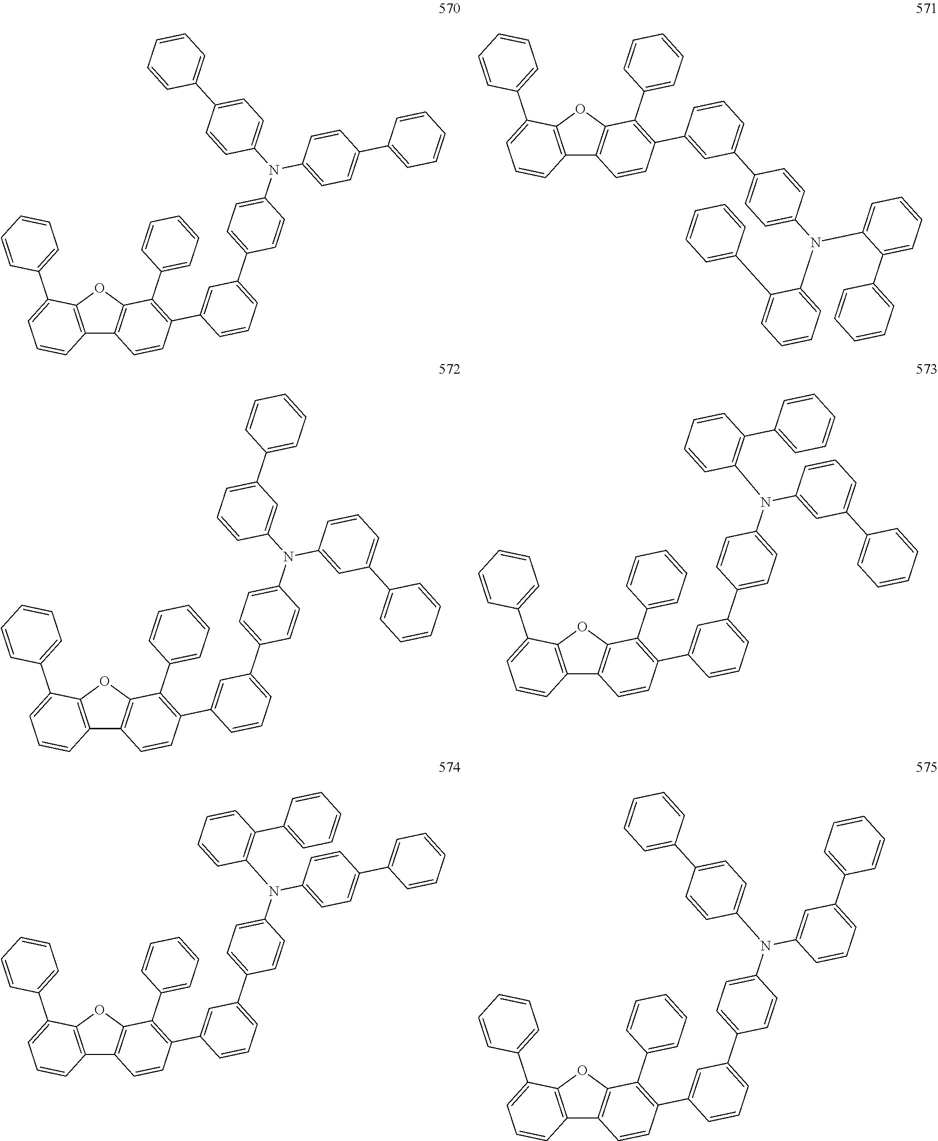

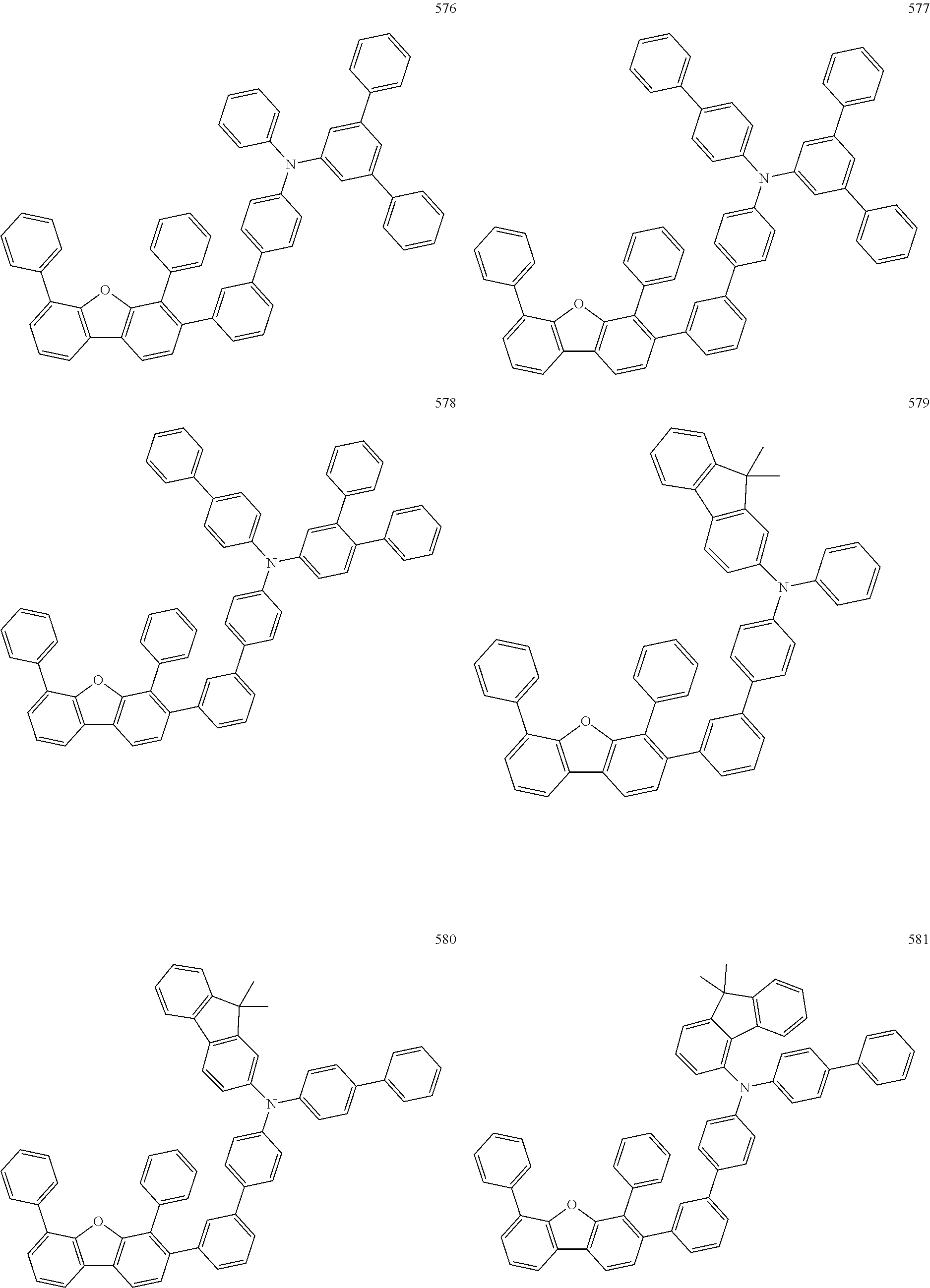

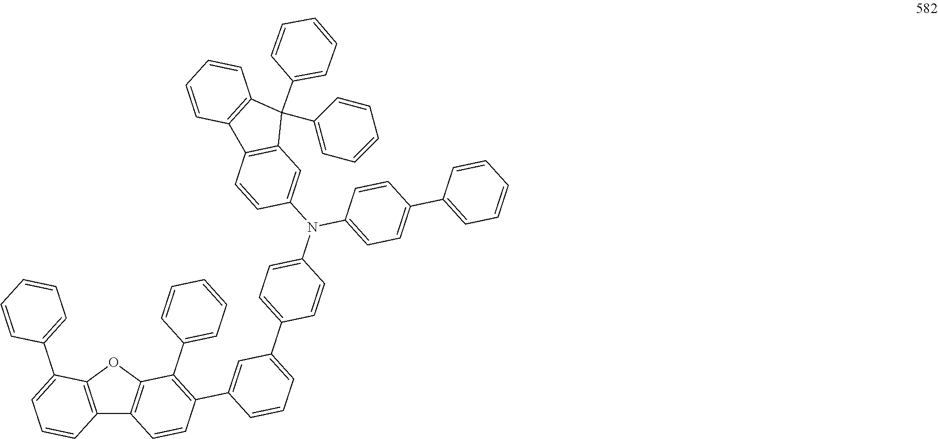 [SMILES: c1ccc(-c2ccc(N(c3ccc(-c4cccc(-c5ccc6c(oc7c(-c8ccccc8)cccc76)c5-c5ccccc5)c4)cc3)c3ccc4c(c3)C(c3ccccc3)(c3ccccc3)c3ccccc3-4)cc2)cc1]

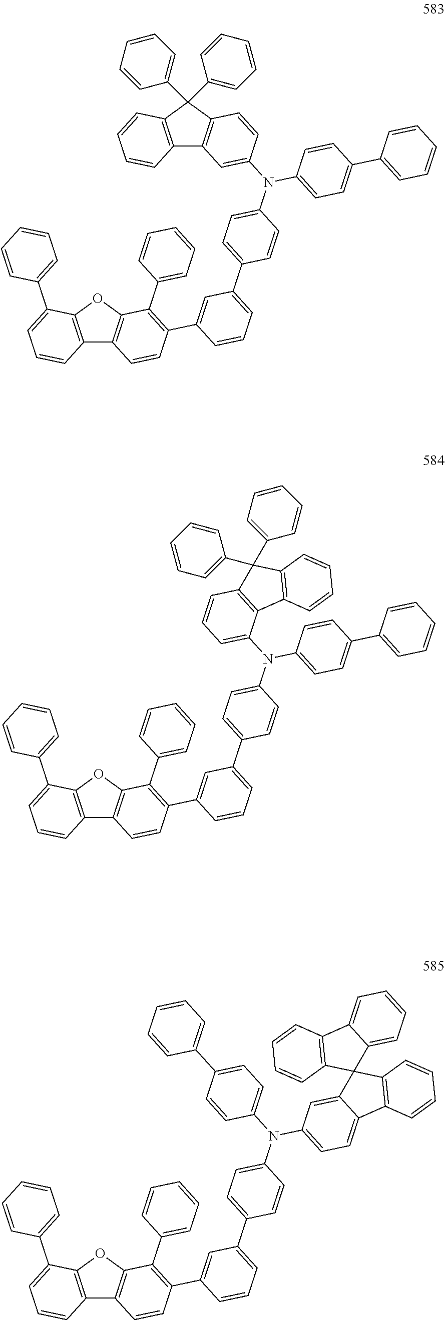 [SMILES: c1ccc(-c2ccc(N(c3ccc(-c4cccc(-c5ccc6c(oc7c(-c8ccccc8)cccc76)c5-c5ccccc5)c4)cc3)c3ccc4c(c3)-c3ccccc3C4(c3ccccc3)c3ccccc3)cc2)cc1 ; c1ccc(-c2ccc(N(c3ccc(-c4cccc(-c5ccc6c(oc7c(-c8ccccc8)cccc76)c5-c5ccccc5)c4)cc3)c3cccc4c3-c3ccccc3C4(c3ccccc3)c3ccccc3)cc2)cc1 ; c1ccc(-c2ccc(N(c3ccc(-c4cccc(-c5ccc6c(oc7c(-c8ccccc8)cccc76)c5-c5ccccc5)c4)cc3)c3ccc4c(c3)C3(c5ccccc5-c5ccccc53)c3ccccc3-4)cc2)cc1]

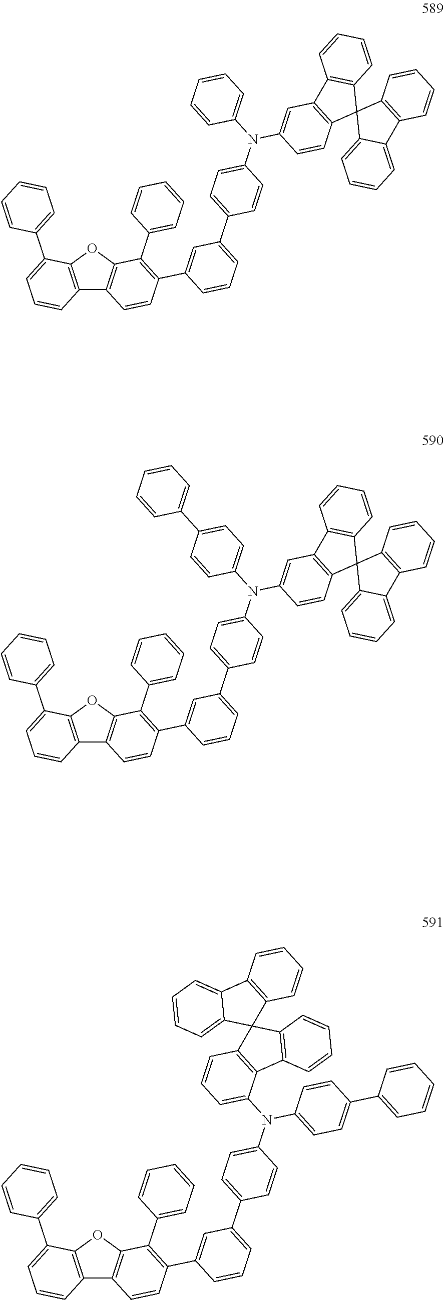 [SMILES: c1ccc(-c2cccc3c2oc2c(-c4ccccc4)c(-c4cccc(-c5ccc(N(c6ccccc6)c6ccc7c(c6)-c6ccccc6C76c7ccccc7-c7ccccc76)cc5)c4)ccc23)cc1 ; c1ccc(-c2ccc(N(c3ccc(-c4cccc(-c5ccc6c(oc7c(-c8ccccc8)cccc76)c5-c5ccccc5)c4)cc3)c3ccc4c(c3)-c3ccccc3C43c4ccccc4-c4ccccc43)cc2)cc1 ; c1ccc(-c2ccc(N(c3ccc(-c4cccc(-c5ccc6c(oc7c(-c8ccccc8)cccc76)c5-c5ccccc5)c4)cc3)c3cccc4c3-c3ccccc3C43c4ccccc4-c4ccccc43)cc2)cc1]

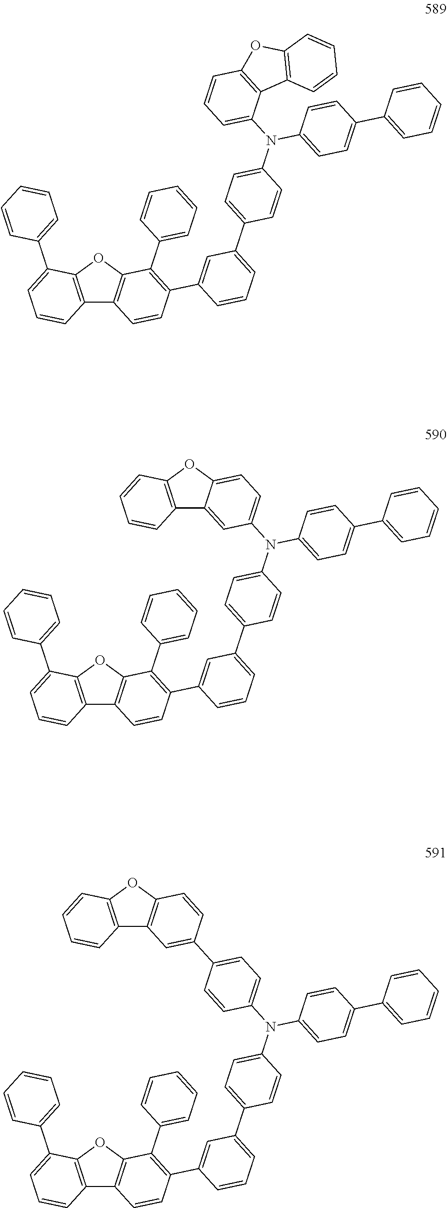 [SMILES: c1ccc(-c2ccc(N(c3ccc(-c4cccc(-c5ccc6c(oc7c(-c8ccccc8)cccc76)c5-c5ccccc5)c4)cc3)c3cccc4oc5ccccc5c34)cc2)cc1 ; c1ccc(-c2ccc(N(c3ccc(-c4cccc(-c5ccc6c(oc7c(-c8ccccc8)cccc76)c5-c5ccccc5)c4)cc3)c3ccc4oc5ccccc5c4c3)cc2)cc1 ; c1ccc(-c2ccc(N(c3ccc(-c4cccc(-c5ccc6c(oc7c(-c8ccccc8)cccc76)c5-c5ccccc5)c4)cc3)c3ccc(-c4ccc5oc6ccccc6c5c4)cc3)cc2)cc1]

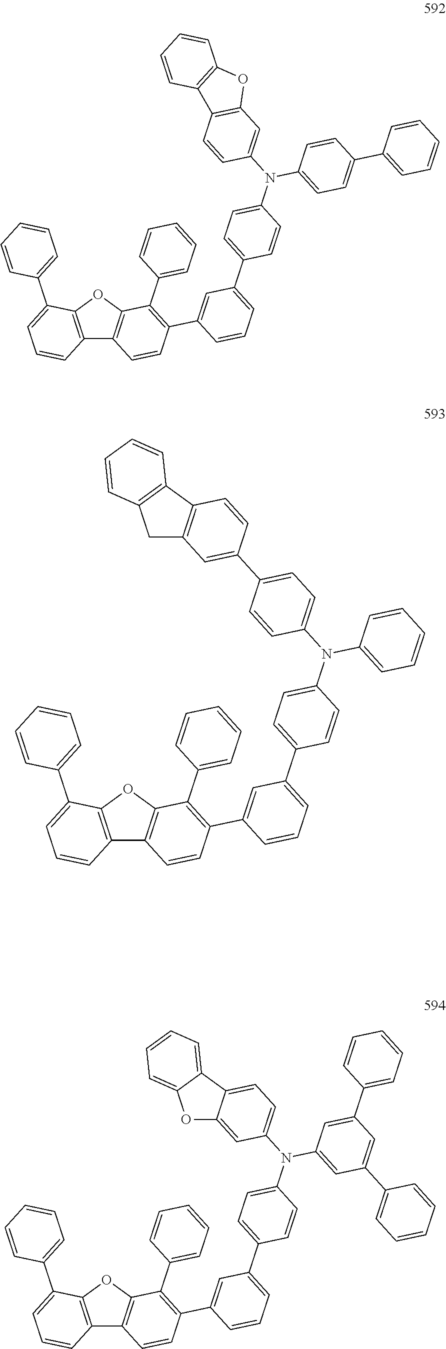 [SMILES: c1ccc(-c2ccc(N(c3ccc(-c4cccc(-c5ccc6c(oc7c(-c8ccccc8)cccc76)c5-c5ccccc5)c4)cc3)c3ccc4c(c3)oc3ccccc34)cc2)cc1 ; c1ccc(-c2cccc3c2oc2c(-c4ccccc4)c(-c4cccc(-c5ccc(N(c6ccccc6)c6ccc(-c7ccc8c(c7)Cc7ccccc7-8)cc6)cc5)c4)ccc23)cc1 ; c1ccc(-c2cc(-c3ccccc3)cc(N(c3ccc(-c4cccc(-c5ccc6c(oc7c(-c8ccccc8)cccc76)c5-c5ccccc5)c4)cc3)c3ccc4c(c3)oc3ccccc34)c2)cc1]

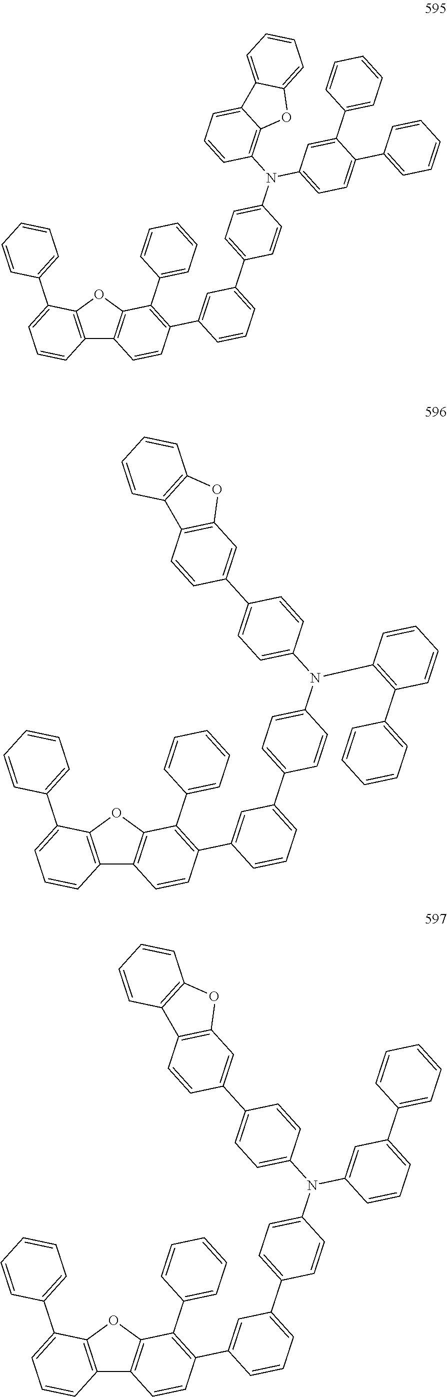 [SMILES: c1ccc(-c2ccc(N(c3ccc(-c4cccc(-c5ccc6c(oc7c(-c8ccccc8)cccc76)c5-c5ccccc5)c4)cc3)c3cccc4c3oc3ccccc34)cc2-c2ccccc2)cc1 ; c1ccc(-c2ccccc2N(c2ccc(-c3cccc(-c4ccc5c(oc6c(-c7ccccc7)cccc65)c4-c4ccccc4)c3)cc2)c2ccc(-c3ccc4c(c3)oc3ccccc34)cc2)cc1 ; c1ccc(-c2cccc(N(c3ccc(-c4cccc(-c5ccc6c(oc7c(-c8ccccc8)cccc76)c5-c5ccccc5)c4)cc3)c3ccc(-c4ccc5c(c4)oc4ccccc45)cc3)c2)cc1]

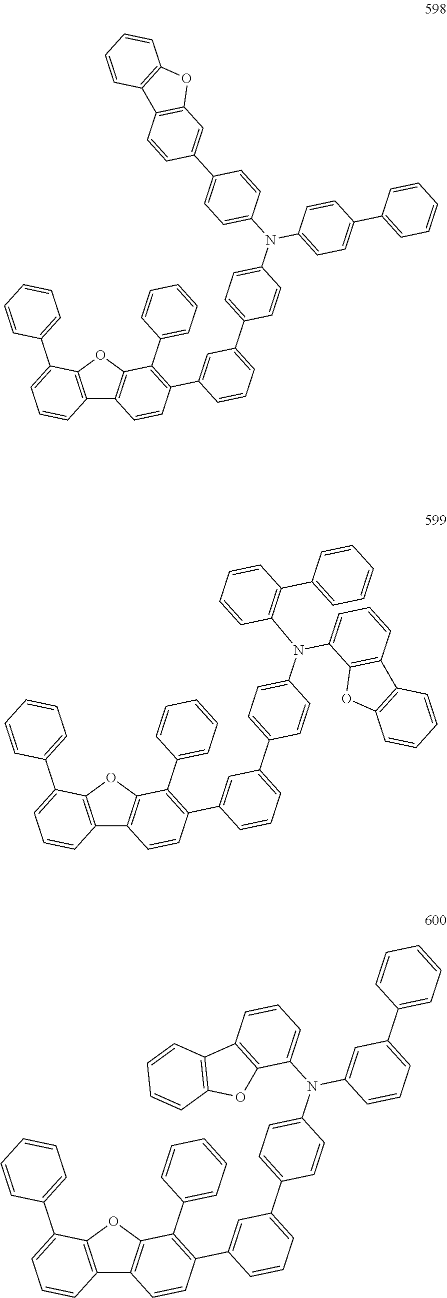 [SMILES: c1ccc(-c2ccc(N(c3ccc(-c4cccc(-c5ccc6c(oc7c(-c8ccccc8)cccc76)c5-c5ccccc5)c4)cc3)c3ccc(-c4ccc5c(c4)oc4ccccc45)cc3)cc2)cc1 ; c1ccc(-c2ccccc2N(c2ccc(-c3cccc(-c4ccc5c(oc6c(-c7ccccc7)cccc65)c4-c4ccccc4)c3)cc2)c2cccc3c2oc2ccccc23)cc1 ; c1ccc(-c2cccc(N(c3ccc(-c4cccc(-c5ccc6c(oc7c(-c8ccccc8)cccc76)c5-c5ccccc5)c4)cc3)c3cccc4c3oc3ccccc34)c2)cc1]

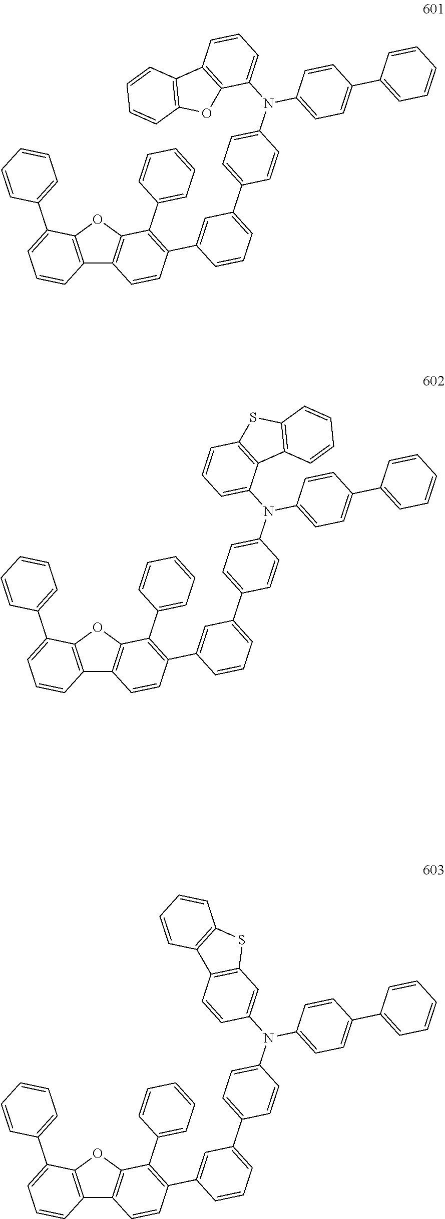 [SMILES: c1ccc(-c2ccc(N(c3ccc(-c4cccc(-c5ccc6c(oc7c(-c8ccccc8)cccc76)c5-c5ccccc5)c4)cc3)c3cccc4c3oc3ccccc34)cc2)cc1 ; c1ccc(-c2ccc(N(c3ccc(-c4cccc(-c5ccc6c(oc7c(-c8ccccc8)cccc76)c5-c5ccccc5)c4)cc3)c3cccc4sc5ccccc5c34)cc2)cc1 ; c1ccc(-c2ccc(N(c3ccc(-c4cccc(-c5ccc6c(oc7c(-c8ccccc8)cccc76)c5-c5ccccc5)c4)cc3)c3ccc4c(c3)sc3ccccc34)cc2)cc1]

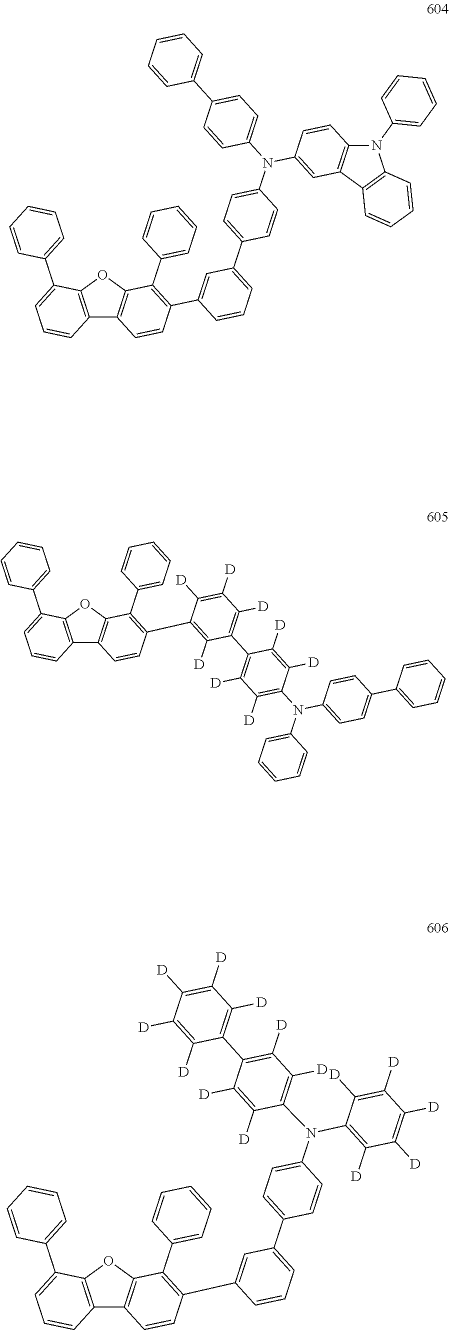 [SMILES: c1ccc(-c2ccc(N(c3ccc(-c4cccc(-c5ccc6c(oc7c(-c8ccccc8)cccc76)c5-c5ccccc5)c4)cc3)c3ccc4c(c3)c3ccccc3n4-c3ccccc3)cc2)cc1 ; [2H]c1c([2H])c(-c2ccc3c(oc4c(-c5ccccc5)cccc43)c2-c2ccccc2)c([2H])c(-c2c([2H])c([2H])c(N(c3ccccc3)c3ccc(-c4ccccc4)cc3)c([2H])c2[2H])c1[2H] ; [2H]c1c([2H])c([2H])c(-c2c([2H])c([2H])c(N(c3ccc(-c4cccc(-c5ccc6c(oc7c(-c8ccccc8)cccc76)c5-c5ccccc5)c4)cc3)c3c([2H])c([2H])c([2H])c([2H])c3[2H])c([2H])c2[2H])c([2H])c1[2H]]

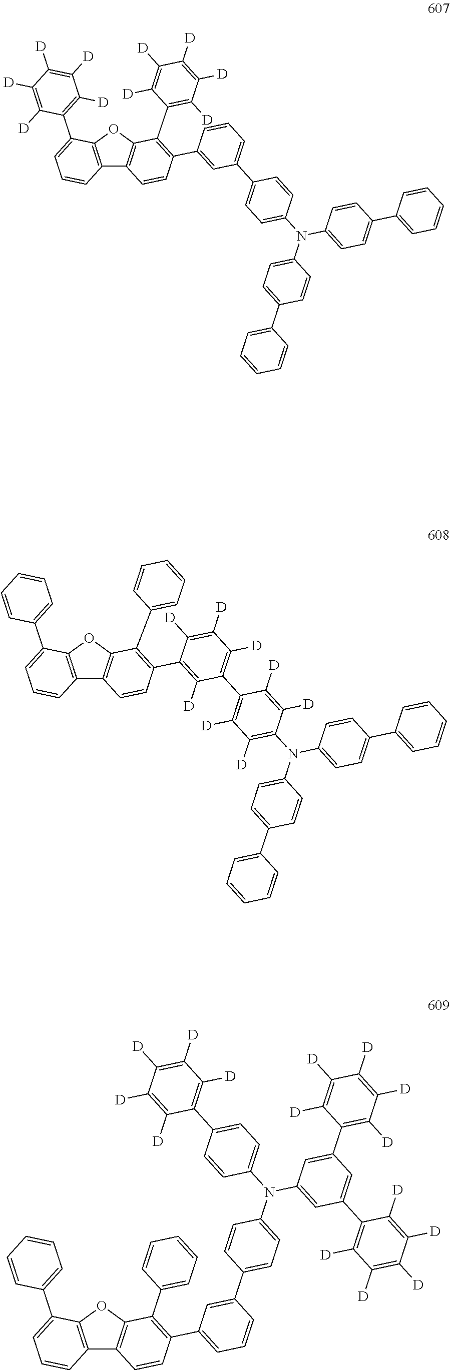

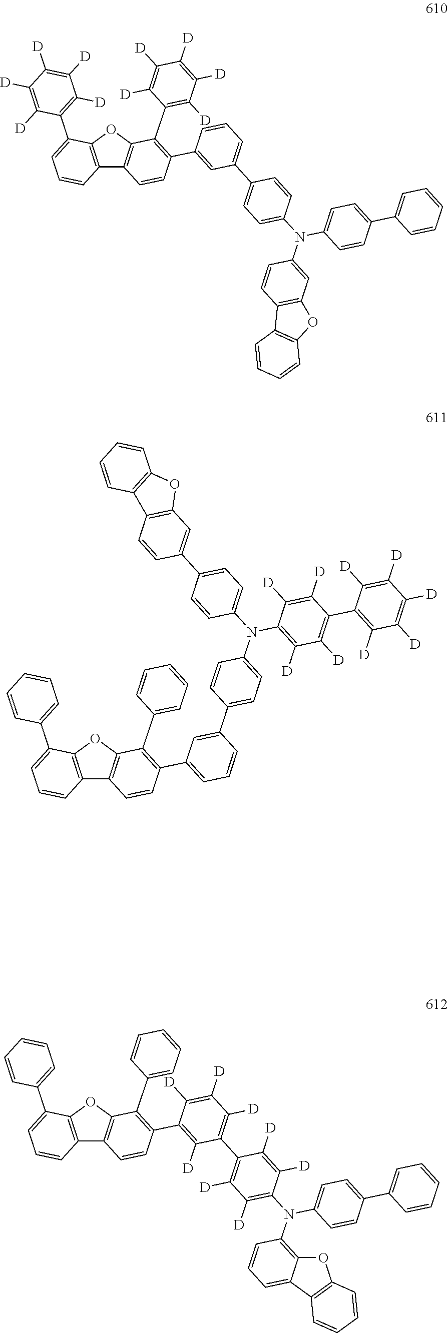 [SMILES: [2H]c1c([2H])c([2H])c(-c2cccc3c2oc2c(-c4c([2H])c([2H])c([2H])c([2H])c4[2H])c(-c4cccc(-c5ccc(N(c6ccc(-c7ccccc7)cc6)c6ccc7c(c6)oc6ccccc67)cc5)c4)ccc23)c([2H])c1[2H] ; [2H]c1c([2H])c([2H])c(-c2c([2H])c([2H])c(N(c3ccc(-c4cccc(-c5ccc6c(oc7c(-c8ccccc8)cccc76)c5-c5ccccc5)c4)cc3)c3ccc(-c4ccc5c(c4)oc4ccccc45)cc3)c([2H])c2[2H])c([2H])c1[2H] ; [2H]c1c([2H])c(-c2ccc3c(oc4c(-c5ccccc5)cccc43)c2-c2ccccc2)c([2H])c(-c2c([2H])c([2H])c(N(c3ccc(-c4ccccc4)cc3)c3cccc4c3oc3ccccc34)c([2H])c2[2H])c1[2H]]

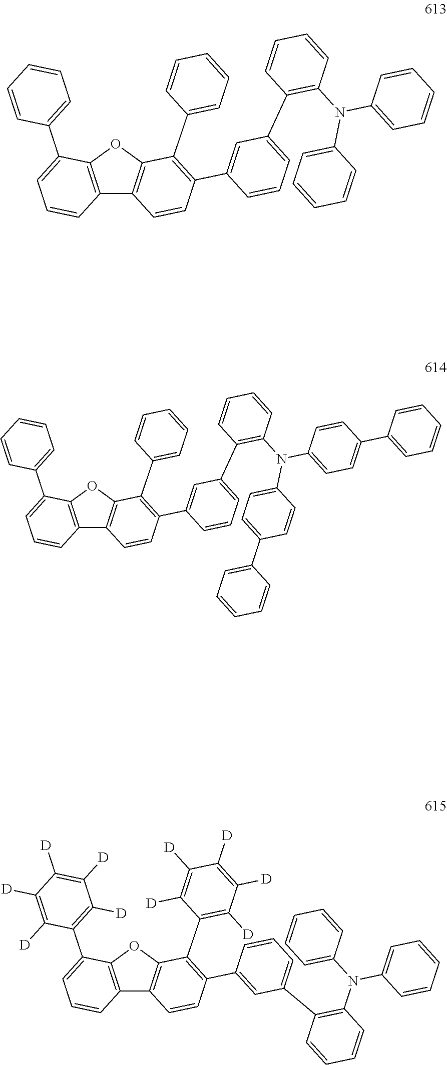 [SMILES: c1ccc(-c2cccc3c2oc2c(-c4ccccc4)c(-c4cccc(-c5ccccc5N(c5ccccc5)c5ccccc5)c4)ccc23)cc1 ; c1ccc(-c2ccc(N(c3ccc(-c4ccccc4)cc3)c3ccccc3-c3cccc(-c4ccc5c(oc6c(-c7ccccc7)cccc65)c4-c4ccccc4)c3)cc2)cc1 ; [2H]c1c([2H])c([2H])c(-c2cccc3c2oc2c(-c4c([2H])c([2H])c([2H])c([2H])c4[2H])c(-c4cccc(-c5ccccc5N(c5ccccc5)c5ccccc5)c4)ccc23)c([2H])c1[2H]]

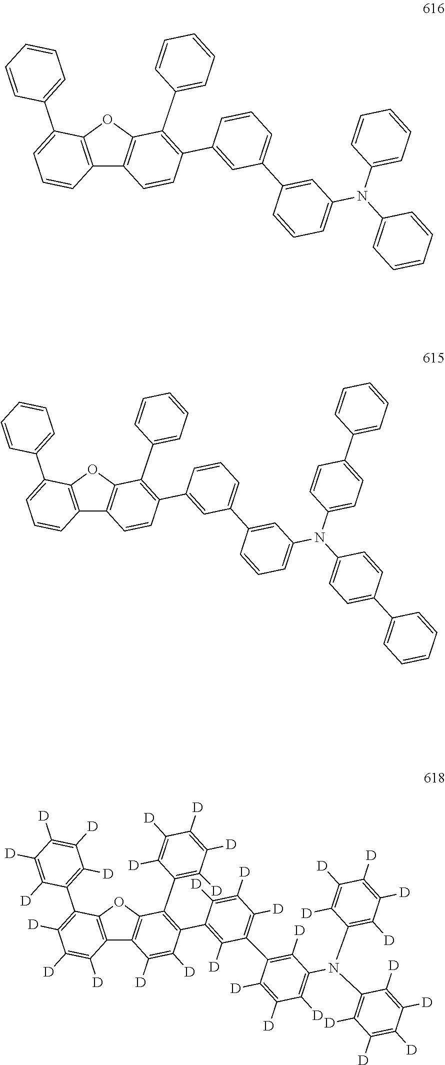 [SMILES: c1ccc(-c2cccc3c2oc2c(-c4ccccc4)c(-c4cccc(-c5cccc(N(c6ccccc6)c6ccccc6)c5)c4)ccc23)cc1 ; c1ccc(-c2ccc(N(c3ccc(-c4ccccc4)cc3)c3cccc(-c4cccc(-c5ccc6c(oc7c(-c8ccccc8)cccc76)c5-c5ccccc5)c4)c3)cc2)cc1 ; [2H]c1c([2H])c([2H])c(-c2c([2H])c([2H])c([2H])c3c2oc2c(-c4c([2H])c([2H])c([2H])c([2H])c4[2H])c(-c4c([2H])c([2H])c([2H])c(-c5c([2H])c([2H])c([2H])c(N(c6c([2H])c([2H])c([2H])c([2H])c6[2H])c6c([2H])c([2H])c([2H])c([2H])c6[2H])c5[2H])c4[2H])c([2H])c([2H])c23)c([2H])c1[2H]]

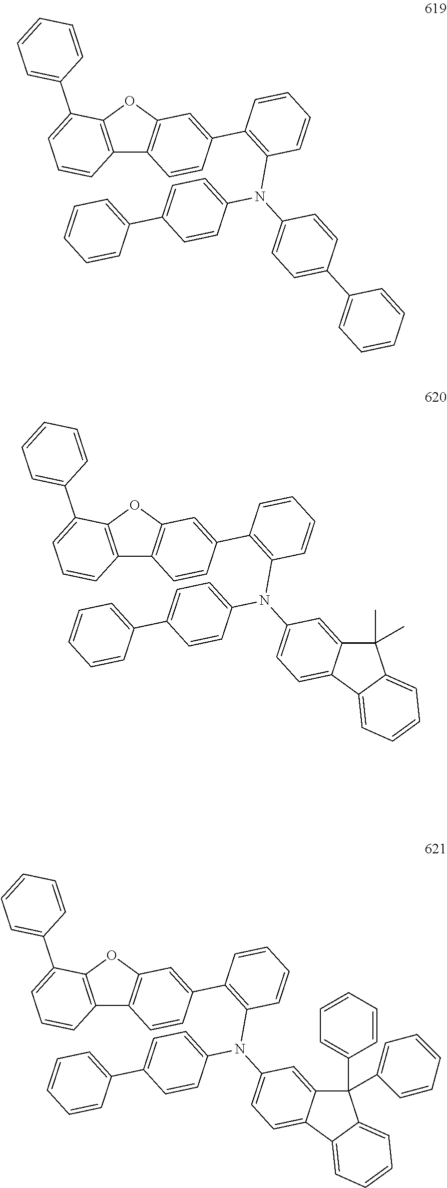 [SMILES: c1ccc(-c2ccc(N(c3ccc(-c4ccccc4)cc3)c3ccccc3-c3ccc4c(c3)oc3c(-c5ccccc5)cccc34)cc2)cc1 ; CC1(C)c2ccccc2-c2ccc(N(c3ccc(-c4ccccc4)cc3)c3ccccc3-c3ccc4c(c3)oc3c(-c5ccccc5)cccc34)cc21 ; c1ccc(-c2ccc(N(c3ccc4c(c3)C(c3ccccc3)(c3ccccc3)c3ccccc3-4)c3ccccc3-c3ccc4c(c3)oc3c(-c5ccccc5)cccc34)cc2)cc1]

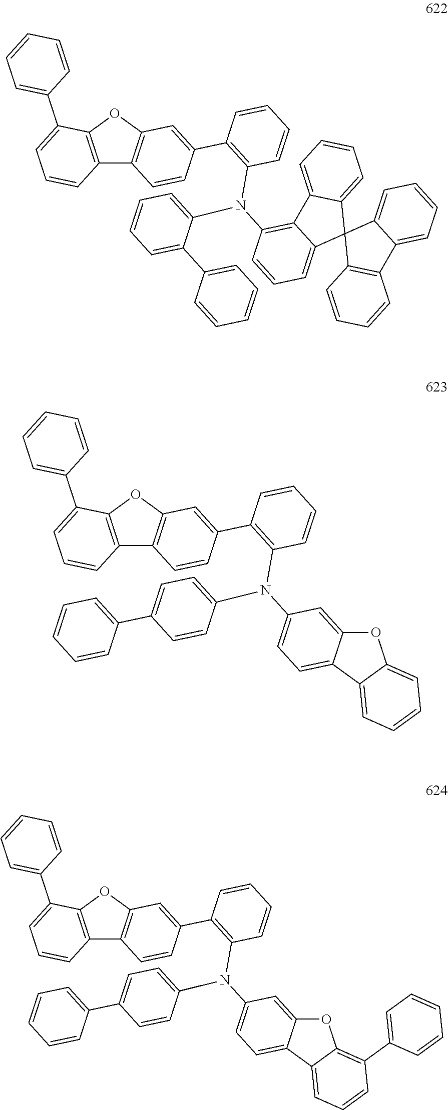 [SMILES: c1ccc(-c2ccccc2N(c2ccccc2-c2ccc3c(c2)oc2c(-c4ccccc4)cccc23)c2cccc3c2-c2ccccc2C32c3ccccc3-c3ccccc32)cc1 ; c1ccc(-c2ccc(N(c3ccc4c(c3)oc3ccccc34)c3ccccc3-c3ccc4c(c3)oc3c(-c5ccccc5)cccc34)cc2)cc1 ; c1ccc(-c2ccc(N(c3ccc4c(c3)oc3c(-c5ccccc5)cccc34)c3ccccc3-c3ccc4c(c3)oc3c(-c5ccccc5)cccc34)cc2)cc1]

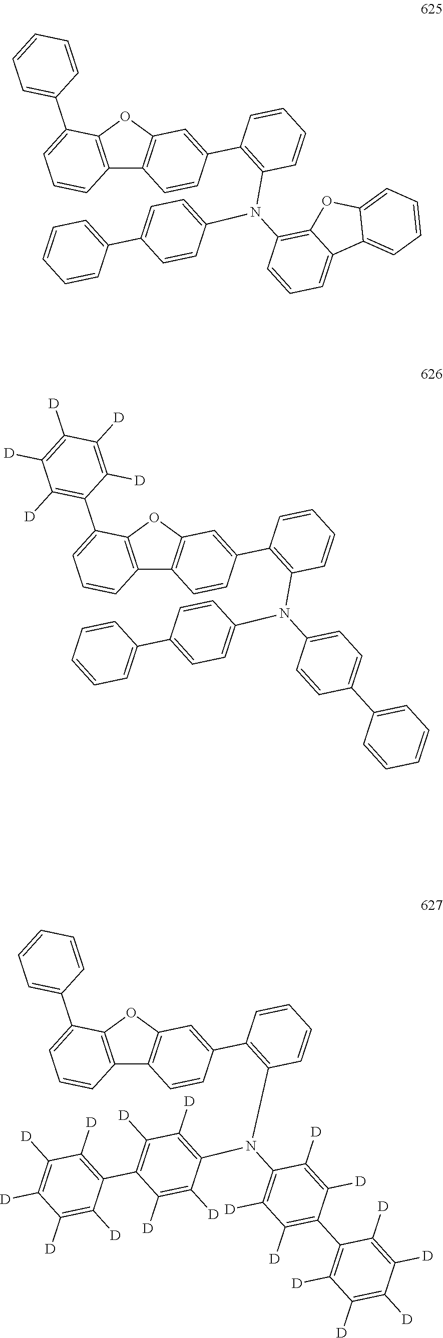 [SMILES: c1ccc(-c2ccc(N(c3ccccc3-c3ccc4c(c3)oc3c(-c5ccccc5)cccc34)c3cccc4c3oc3ccccc34)cc2)cc1 ; [2H]c1c([2H])c([2H])c(-c2cccc3c2oc2cc(-c4ccccc4N(c4ccc(-c5ccccc5)cc4)c4ccc(-c5ccccc5)cc4)ccc23)c([2H])c1[2H] ; [2H]c1c([2H])c([2H])c(-c2c([2H])c([2H])c(N(c3ccccc3-c3ccc4c(c3)oc3c(-c5ccccc5)cccc34)c3c([2H])c([2H])c(-c4c([2H])c([2H])c([2H])c([2H])c4[2H])c([2H])c3[2H])c([2H])c2[2H])c([2H])c1[2H]]

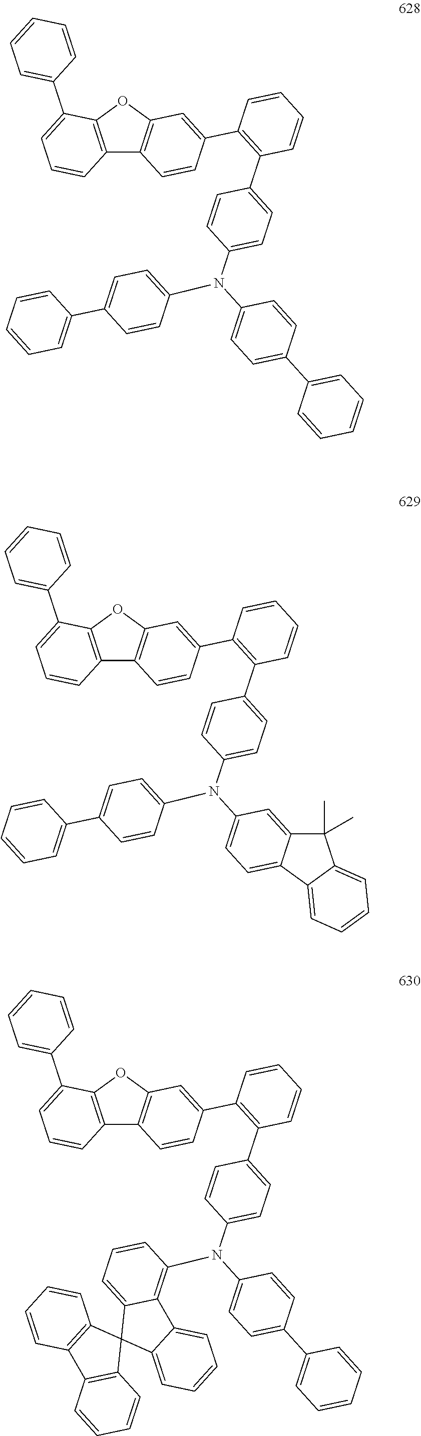 [SMILES: c1ccc(-c2ccc(N(c3ccc(-c4ccccc4)cc3)c3ccc(-c4ccccc4-c4ccc5c(c4)oc4c(-c6ccccc6)cccc45)cc3)cc2)cc1 ; CC1(C)c2ccccc2-c2ccc(N(c3ccc(-c4ccccc4)cc3)c3ccc(-c4ccccc4-c4ccc5c(c4)oc4c(-c6ccccc6)cccc45)cc3)cc21 ; c1ccc(-c2ccc(N(c3ccc(-c4ccccc4-c4ccc5c(c4)oc4c(-c6ccccc6)cccc45)cc3)c3cccc4c3-c3ccccc3C43c4ccccc4-c4ccccc43)cc2)cc1]

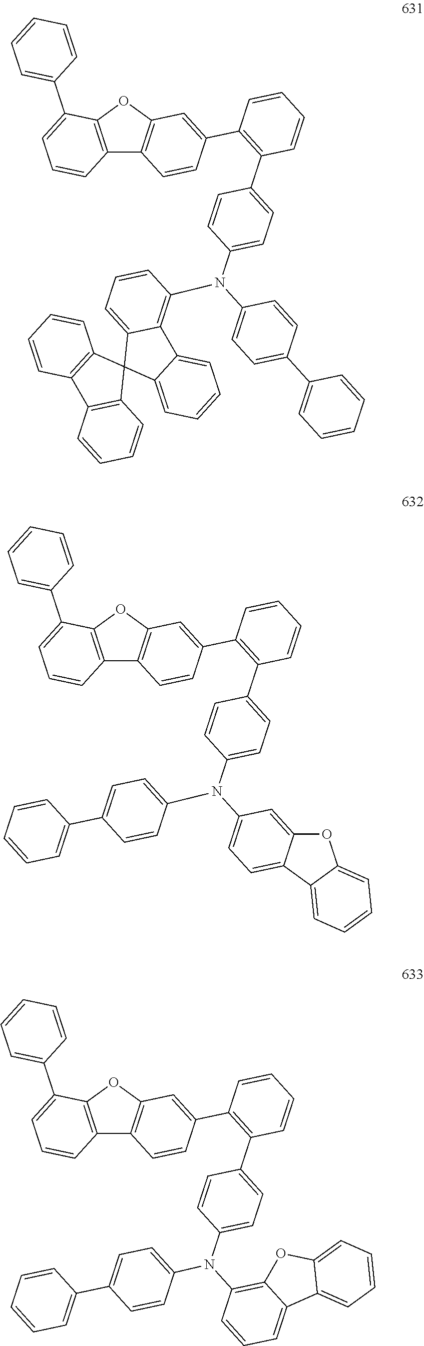 [SMILES: c1ccc(-c2ccc(N(c3ccc(-c4ccccc4-c4ccc5c(c4)oc4c(-c6ccccc6)cccc45)cc3)c3cccc4c3-c3ccccc3C43c4ccccc4-c4ccccc43)cc2)cc1 ; c1ccc(-c2ccc(N(c3ccc(-c4ccccc4-c4ccc5c(c4)oc4c(-c6ccccc6)cccc45)cc3)c3ccc4c(c3)oc3ccccc34)cc2)cc1 ; c1ccc(-c2ccc(N(c3ccc(-c4ccccc4-c4ccc5c(c4)oc4c(-c6ccccc6)cccc45)cc3)c3cccc4c3oc3ccccc34)cc2)cc1]

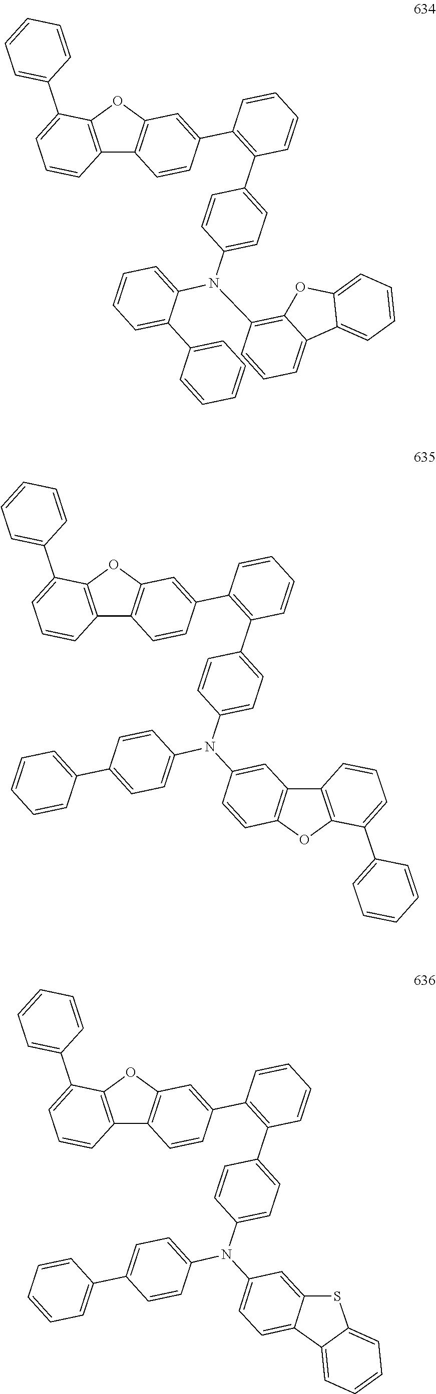 [SMILES: c1ccc(-c2ccccc2N(c2ccc(-c3ccccc3-c3ccc4c(c3)oc3c(-c5ccccc5)cccc34)cc2)c2cccc3c2oc2ccccc23)cc1 ; c1ccc(-c2ccc(N(c3ccc(-c4ccccc4-c4ccc5c(c4)oc4c(-c6ccccc6)cccc45)cc3)c3ccc4oc5c(-c6ccccc6)cccc5c4c3)cc2)cc1 ; c1ccc(-c2ccc(N(c3ccc(-c4ccccc4-c4ccc5c(c4)oc4c(-c6ccccc6)cccc45)cc3)c3ccc4c(c3)sc3ccccc34)cc2)cc1]

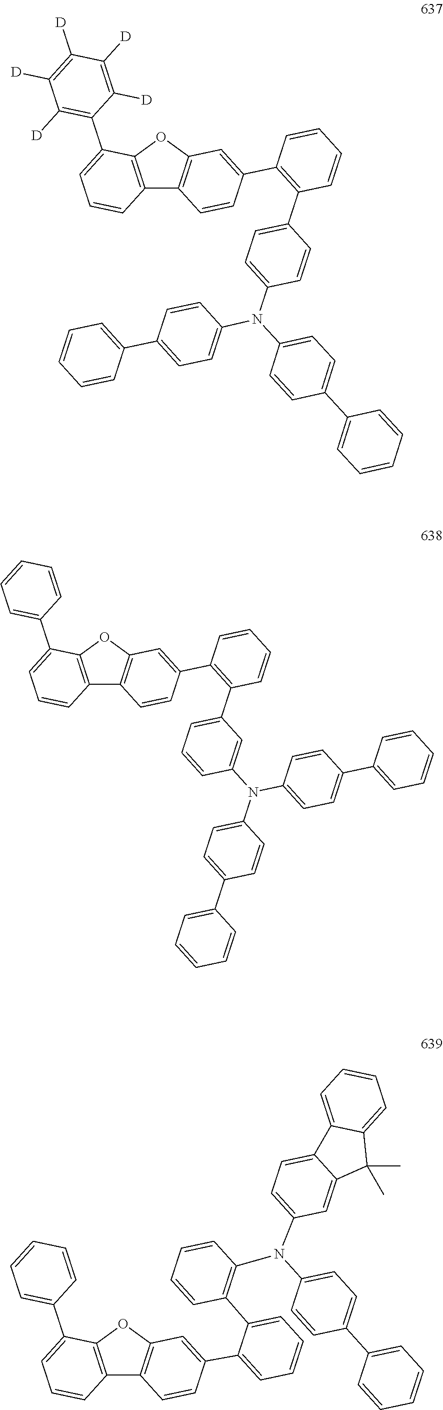 [SMILES: [2H]c1c([2H])c([2H])c(-c2cccc3c2oc2cc(-c4ccccc4-c4ccc(N(c5ccc(-c6ccccc6)cc5)c5ccc(-c6ccccc6)cc5)cc4)ccc23)c([2H])c1[2H] ; c1ccc(-c2ccc(N(c3ccc(-c4ccccc4)cc3)c3cccc(-c4ccccc4-c4ccc5c(c4)oc4c(-c6ccccc6)cccc45)c3)cc2)cc1 ; CC1(C)c2ccccc2-c2ccc(N(c3ccc(-c4ccccc4)cc3)c3ccccc3-c3ccccc3-c3ccc4c(c3)oc3c(-c5ccccc5)cccc34)cc21]

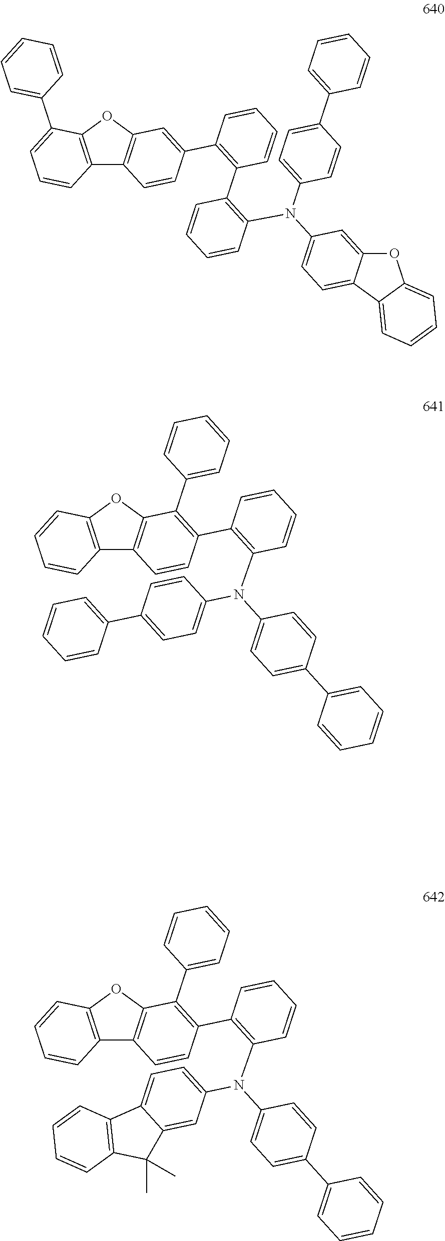 [SMILES: c1ccc(-c2ccc(N(c3ccc4c(c3)oc3ccccc34)c3ccccc3-c3ccccc3-c3ccc4c(c3)oc3c(-c5ccccc5)cccc34)cc2)cc1 ; c1ccc(-c2ccc(N(c3ccc(-c4ccccc4)cc3)c3ccccc3-c3ccc4c(oc5ccccc54)c3-c3ccccc3)cc2)cc1 ; CC1(C)c2ccccc2-c2ccc(N(c3ccc(-c4ccccc4)cc3)c3ccccc3-c3ccc4c(oc5ccccc54)c3-c3ccccc3)cc21]

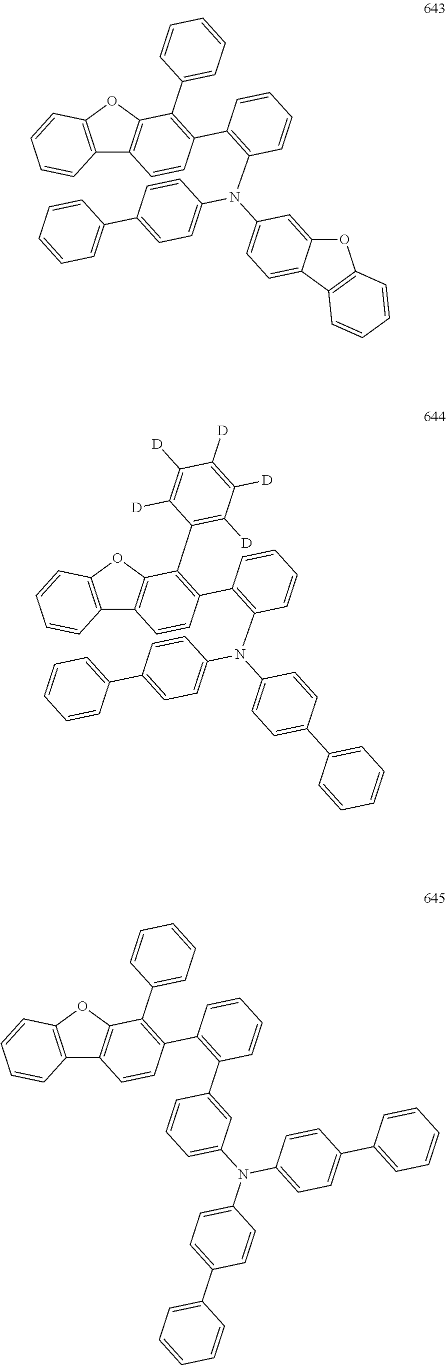 [SMILES: c1ccc(-c2ccc(N(c3ccc4c(c3)oc3ccccc34)c3ccccc3-c3ccc4c(oc5ccccc54)c3-c3ccccc3)cc2)cc1 ; [2H]c1c([2H])c([2H])c(-c2c(-c3ccccc3N(c3ccc(-c4ccccc4)cc3)c3ccc(-c4ccccc4)cc3)ccc3c2oc2ccccc23)c([2H])c1[2H] ; c1ccc(-c2ccc(N(c3ccc(-c4ccccc4)cc3)c3cccc(-c4ccccc4-c4ccc5c(oc6ccccc65)c4-c4ccccc4)c3)cc2)cc1]

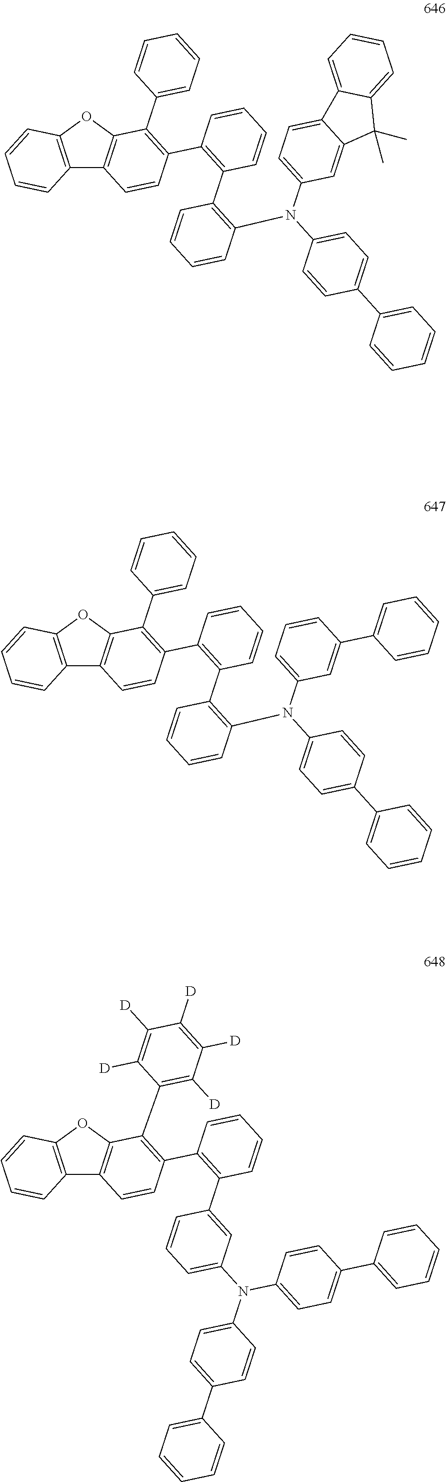 [SMILES: CC1(C)c2ccccc2-c2ccc(N(c3ccc(-c4ccccc4)cc3)c3ccccc3-c3ccccc3-c3ccc4c(oc5ccccc54)c3-c3ccccc3)cc21 ; c1ccc(-c2ccc(N(c3cccc(-c4ccccc4)c3)c3ccccc3-c3ccccc3-c3ccc4c(oc5ccccc54)c3-c3ccccc3)cc2)cc1 ; [2H]c1c([2H])c([2H])c(-c2c(-c3ccccc3-c3cccc(N(c4ccc(-c5ccccc5)cc4)c4ccc(-c5ccccc5)cc4)c3)ccc3c2oc2ccccc23)c([2H])c1[2H]]

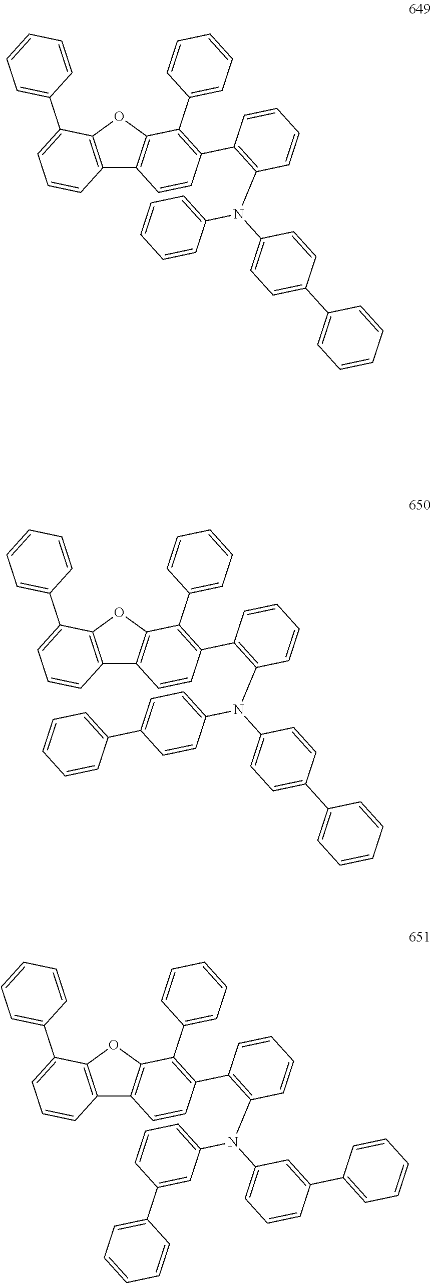 [SMILES: c1ccc(-c2ccc(N(c3ccccc3)c3ccccc3-c3ccc4c(oc5c(-c6ccccc6)cccc54)c3-c3ccccc3)cc2)cc1 ; c1ccc(-c2ccc(N(c3ccc(-c4ccccc4)cc3)c3ccccc3-c3ccc4c(oc5c(-c6ccccc6)cccc54)c3-c3ccccc3)cc2)cc1 ; c1ccc(-c2cccc(N(c3cccc(-c4ccccc4)c3)c3ccccc3-c3ccc4c(oc5c(-c6ccccc6)cccc54)c3-c3ccccc3)c2)cc1]

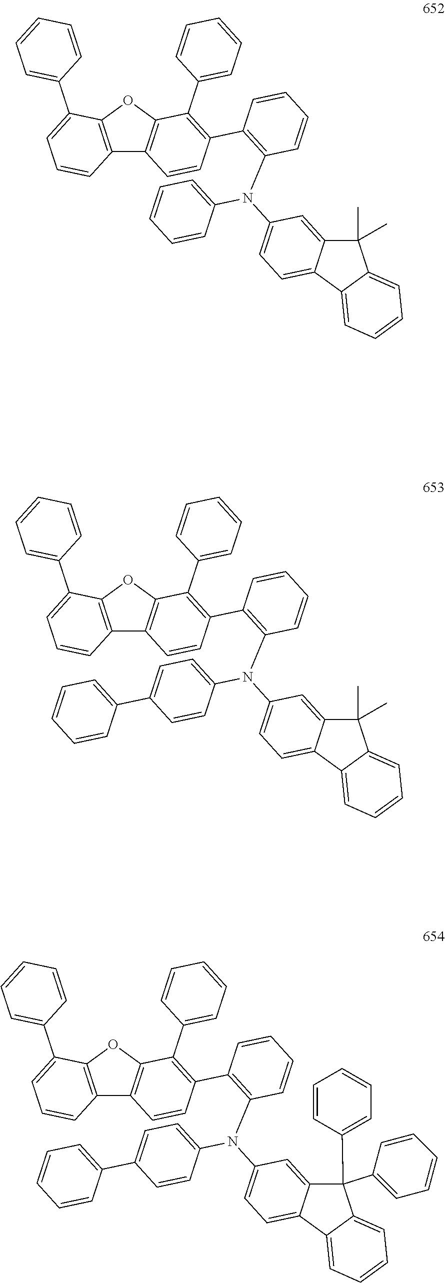 [SMILES: CC1(C)c2ccccc2-c2ccc(N(c3ccccc3)c3ccccc3-c3ccc4c(oc5c(-c6ccccc6)cccc54)c3-c3ccccc3)cc21 ; CC1(C)c2ccccc2-c2ccc(N(c3ccc(-c4ccccc4)cc3)c3ccccc3-c3ccc4c(oc5c(-c6ccccc6)cccc54)c3-c3ccccc3)cc21 ; c1ccc(-c2ccc(N(c3ccc4c(c3)C(c3ccccc3)(c3ccccc3)c3ccccc3-4)c3ccccc3-c3ccc4c(oc5c(-c6ccccc6)cccc54)c3-c3ccccc3)cc2)cc1]

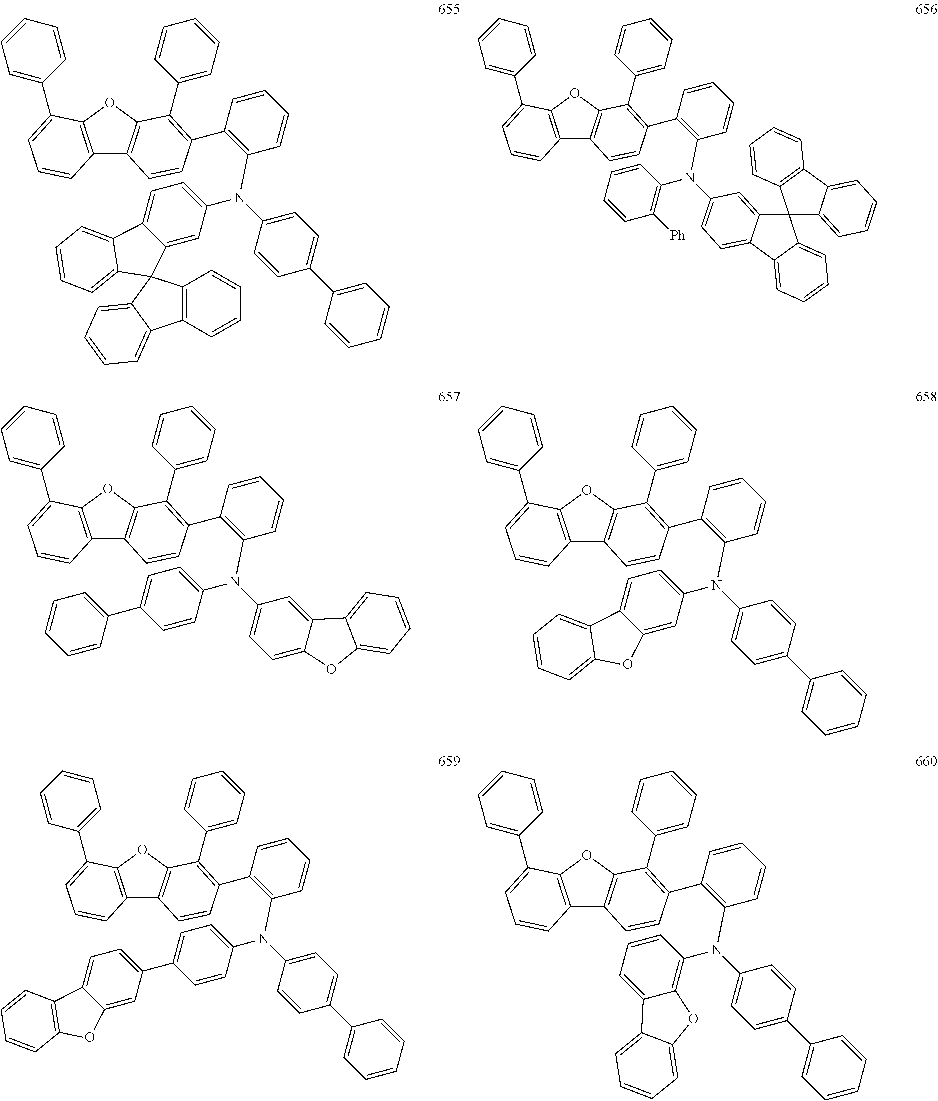

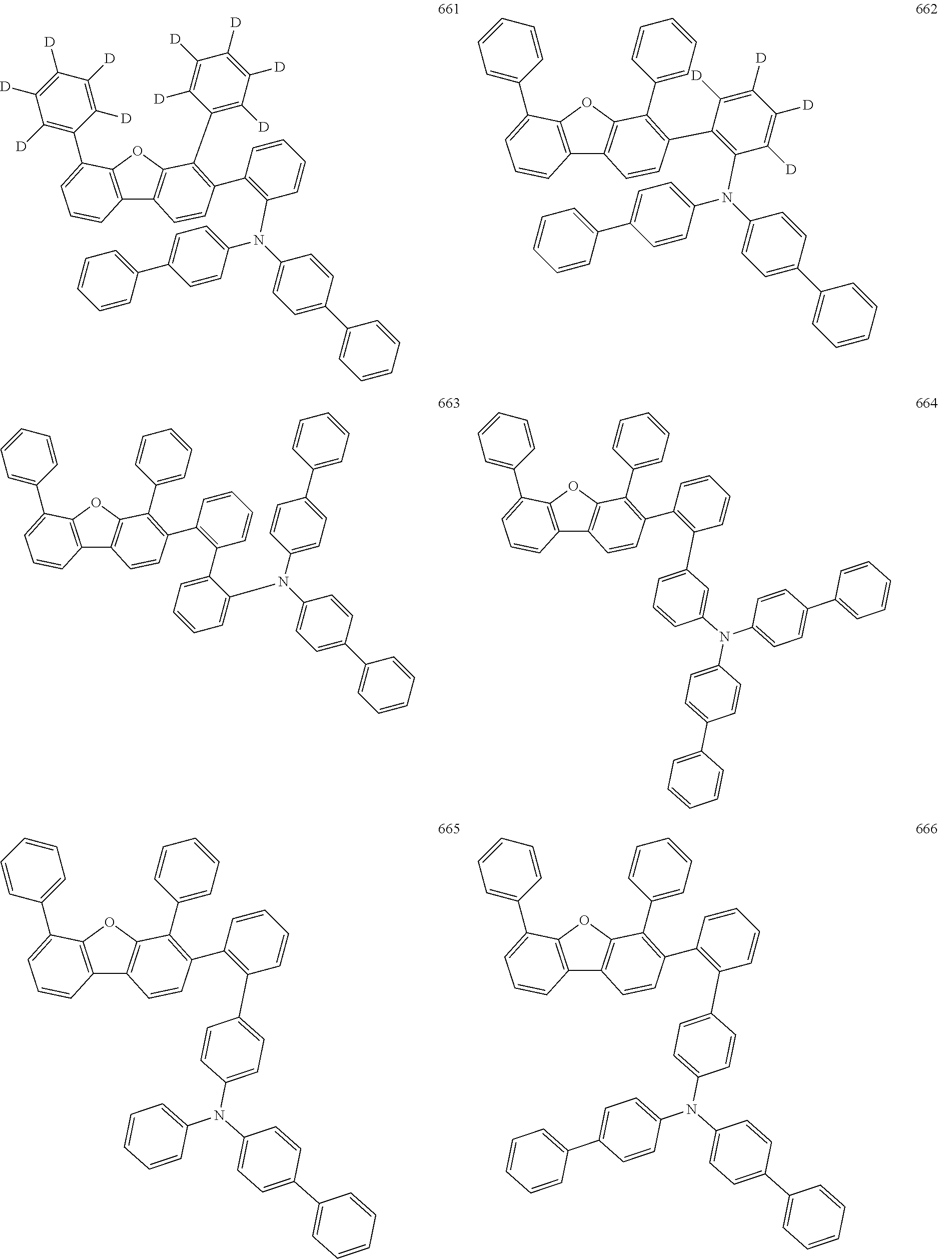

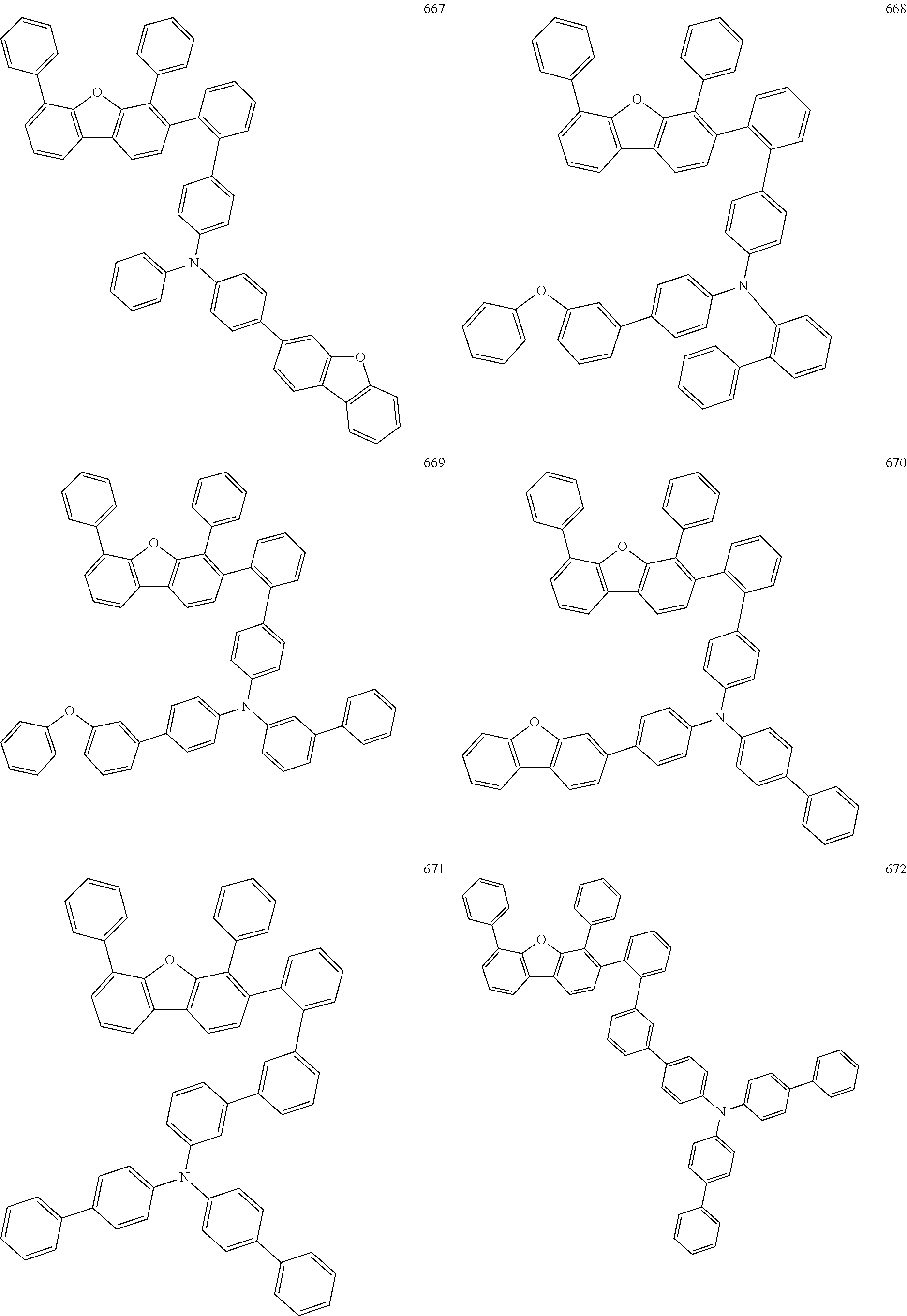

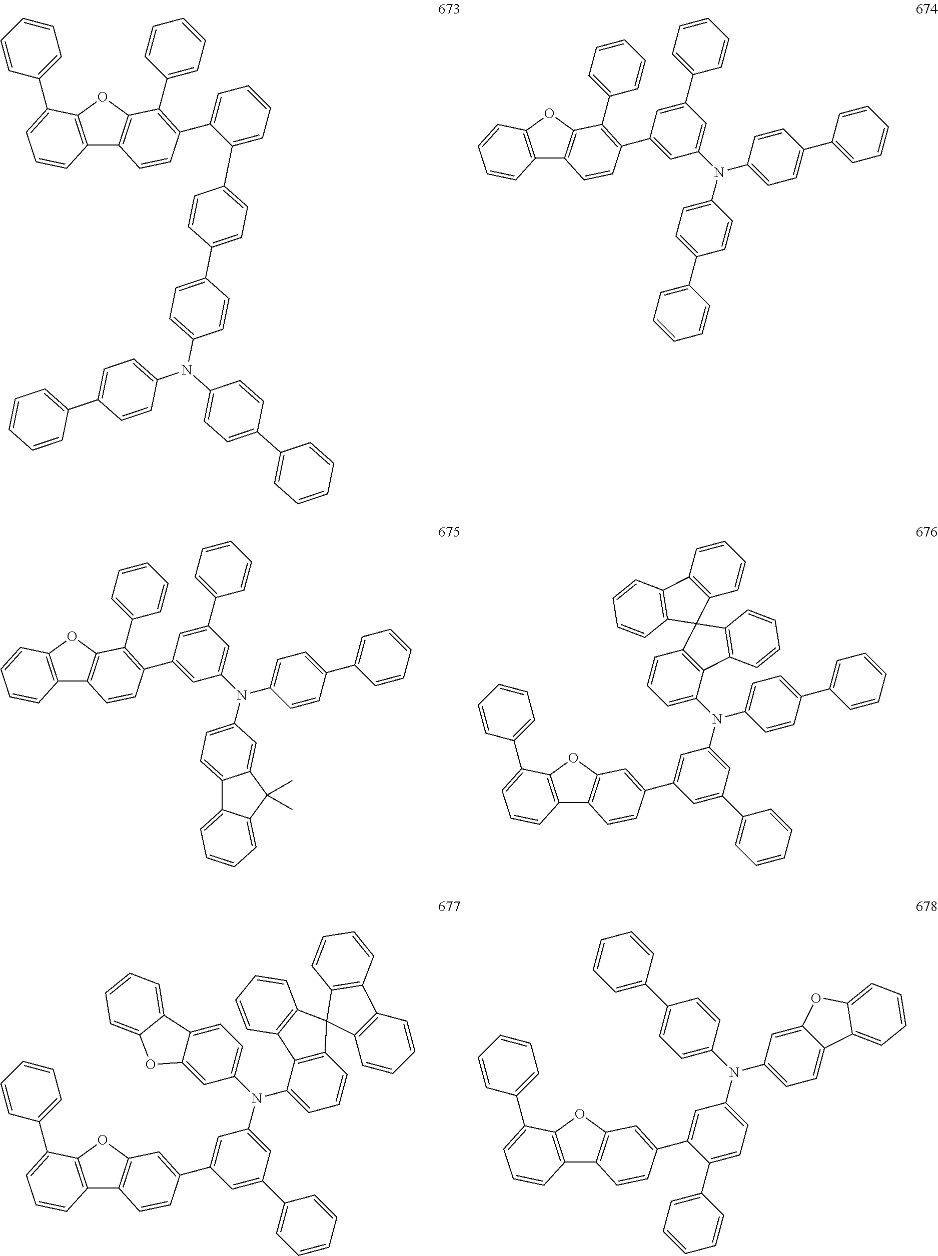

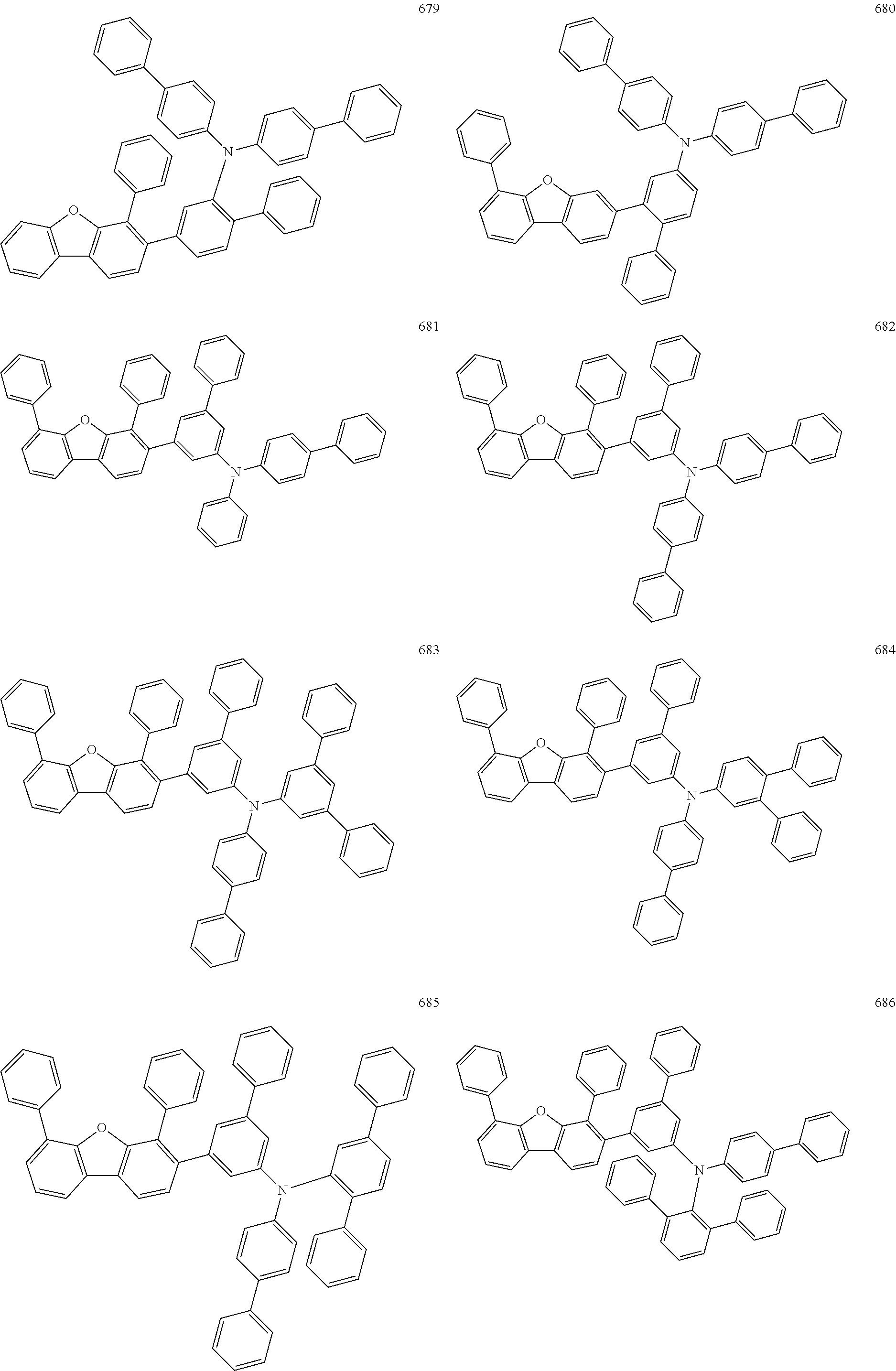

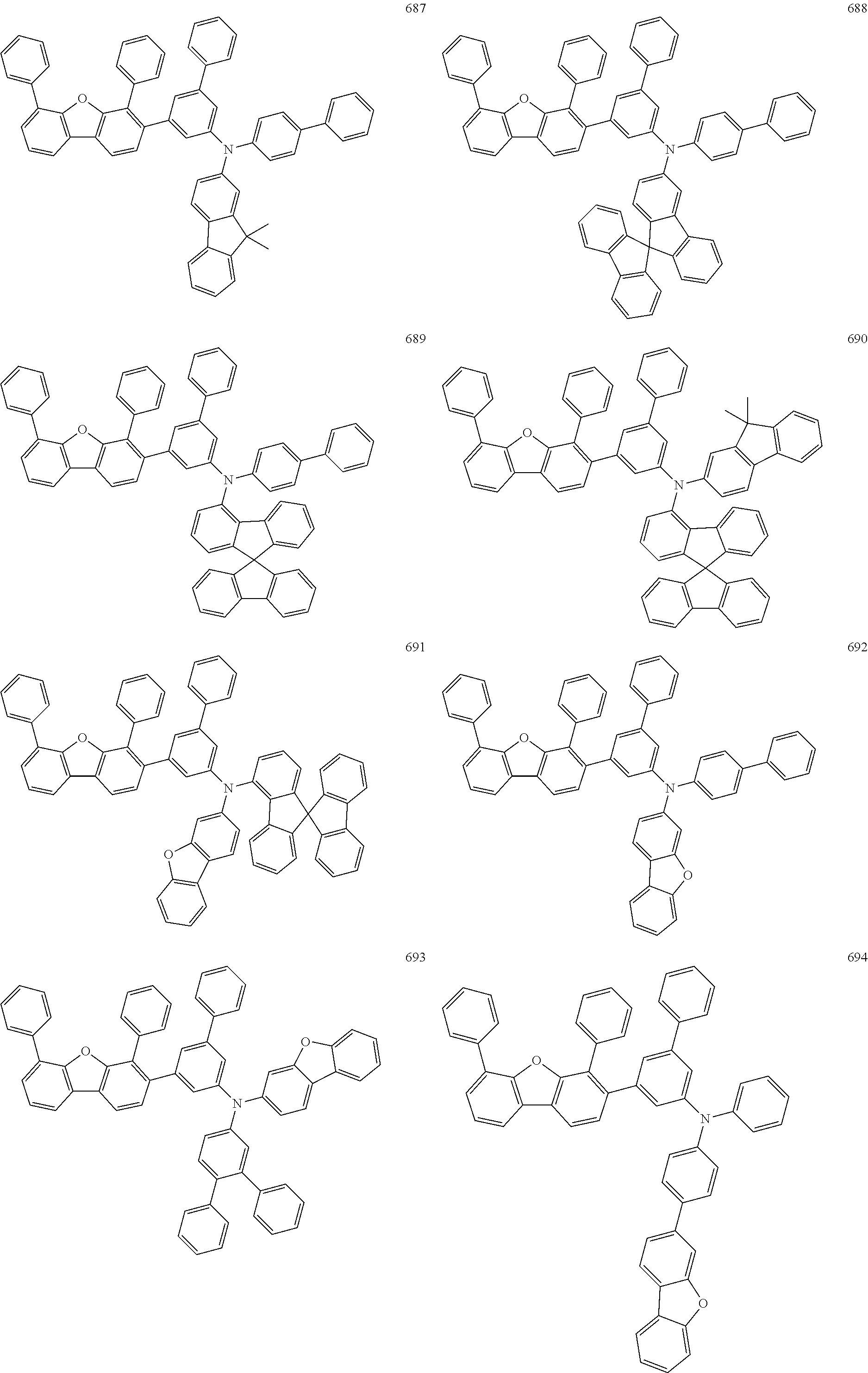

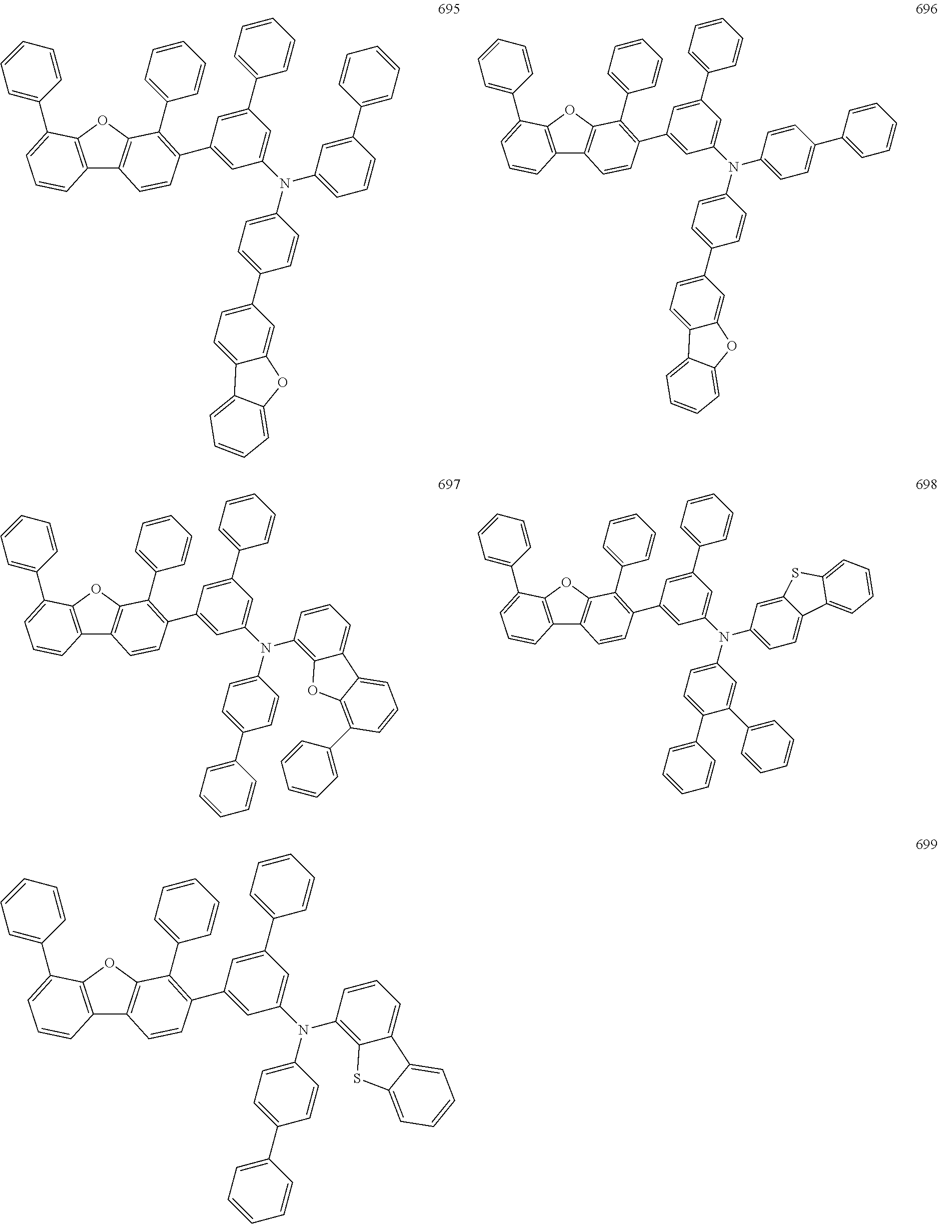

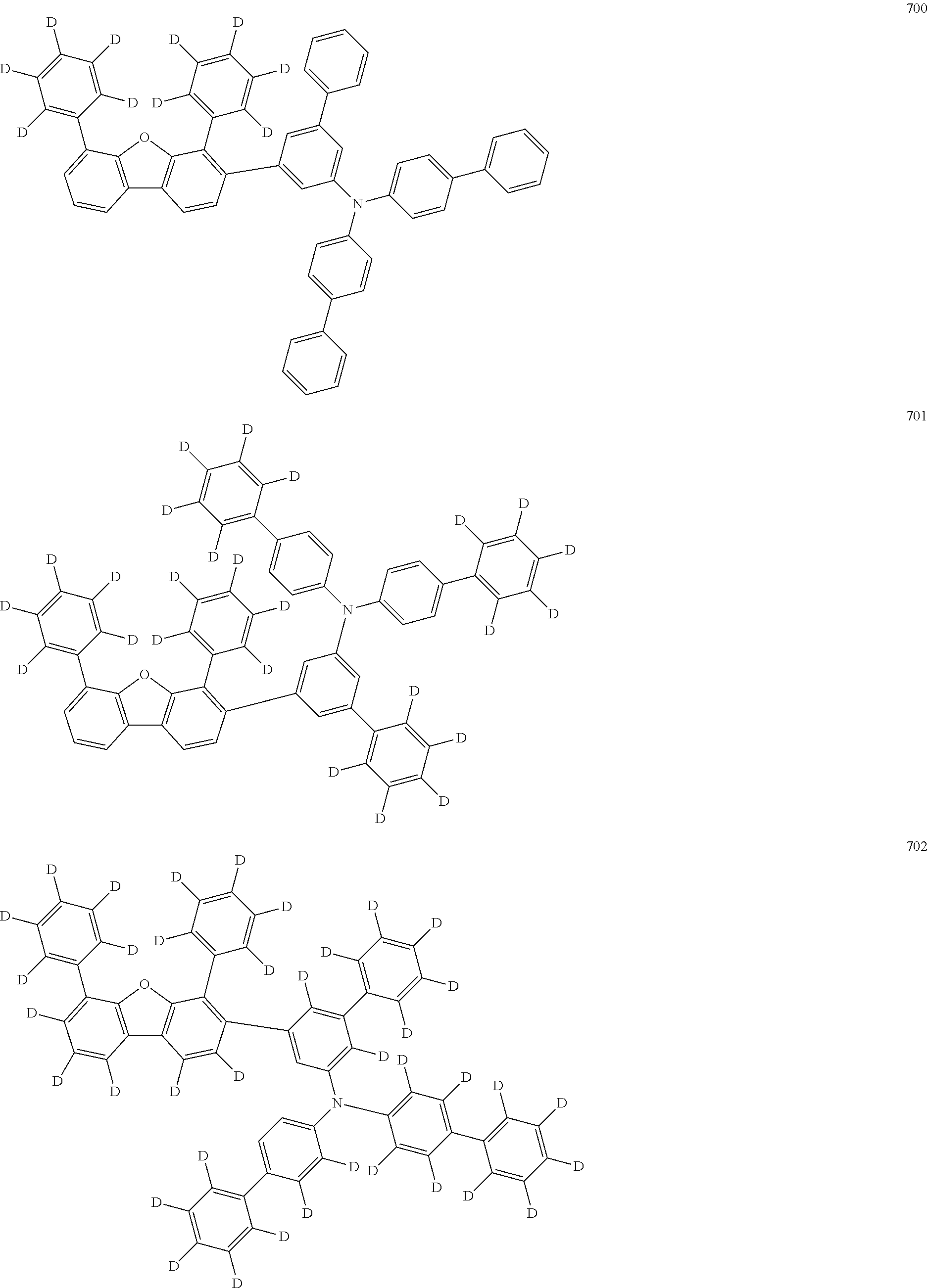

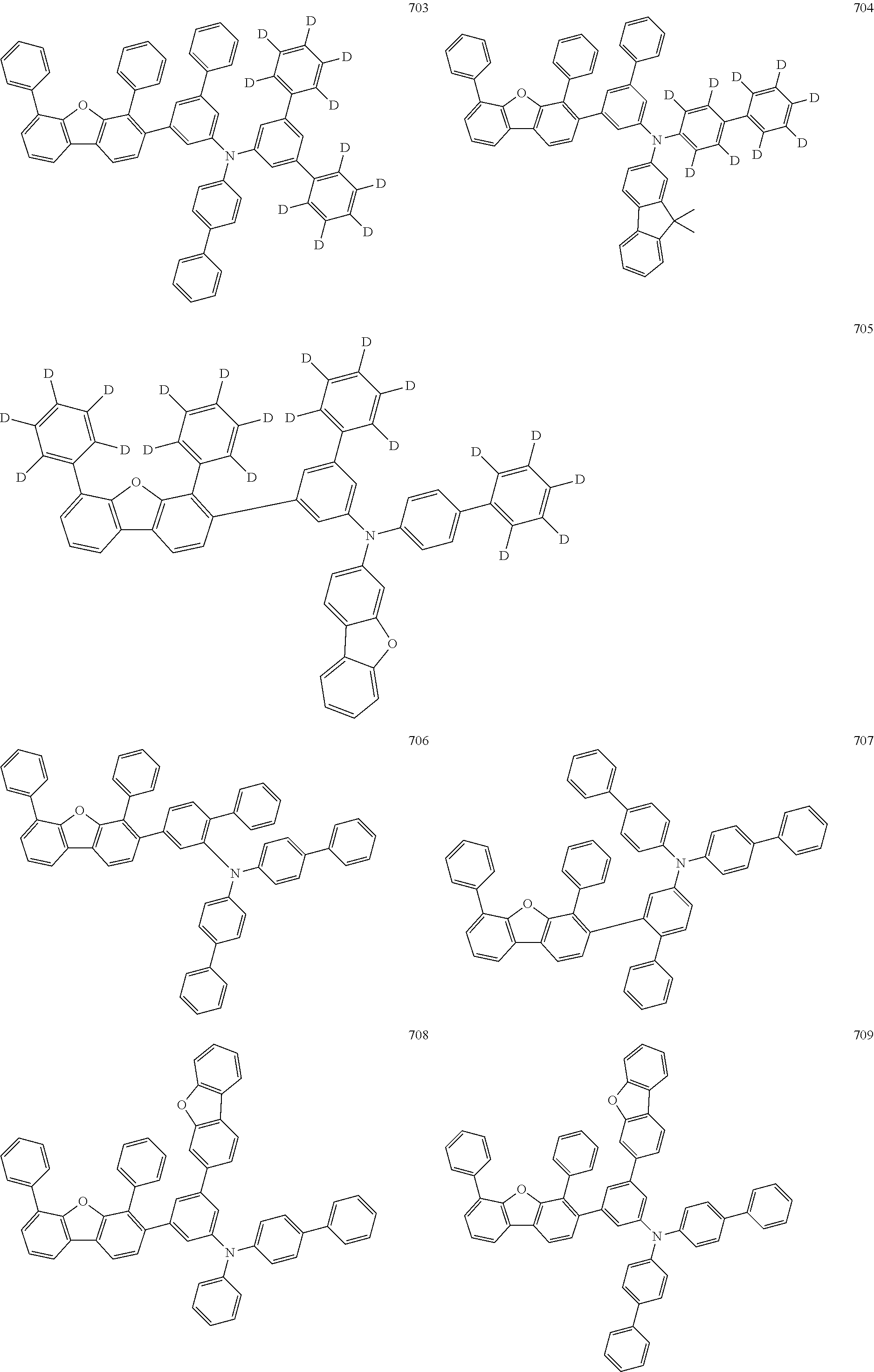

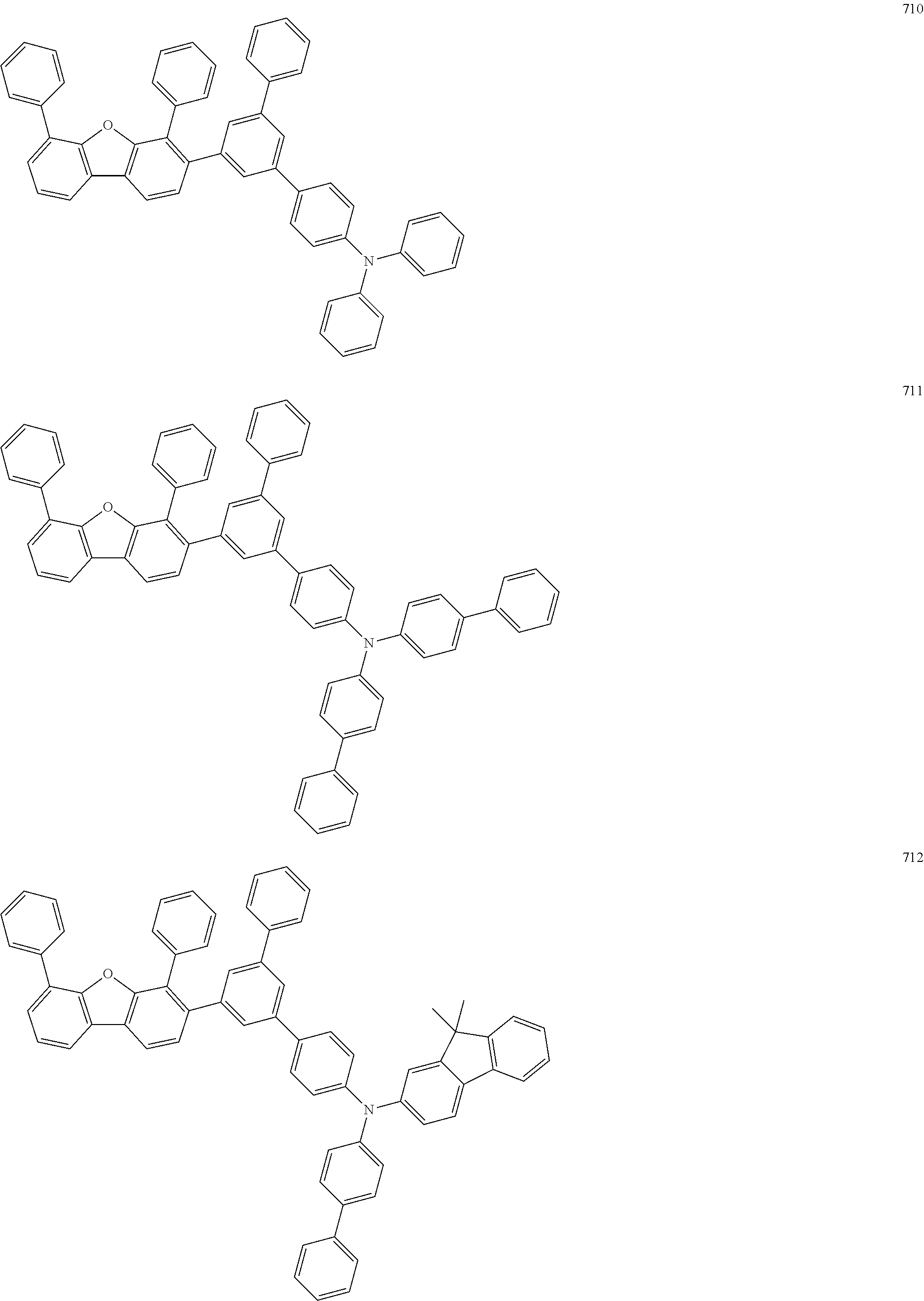

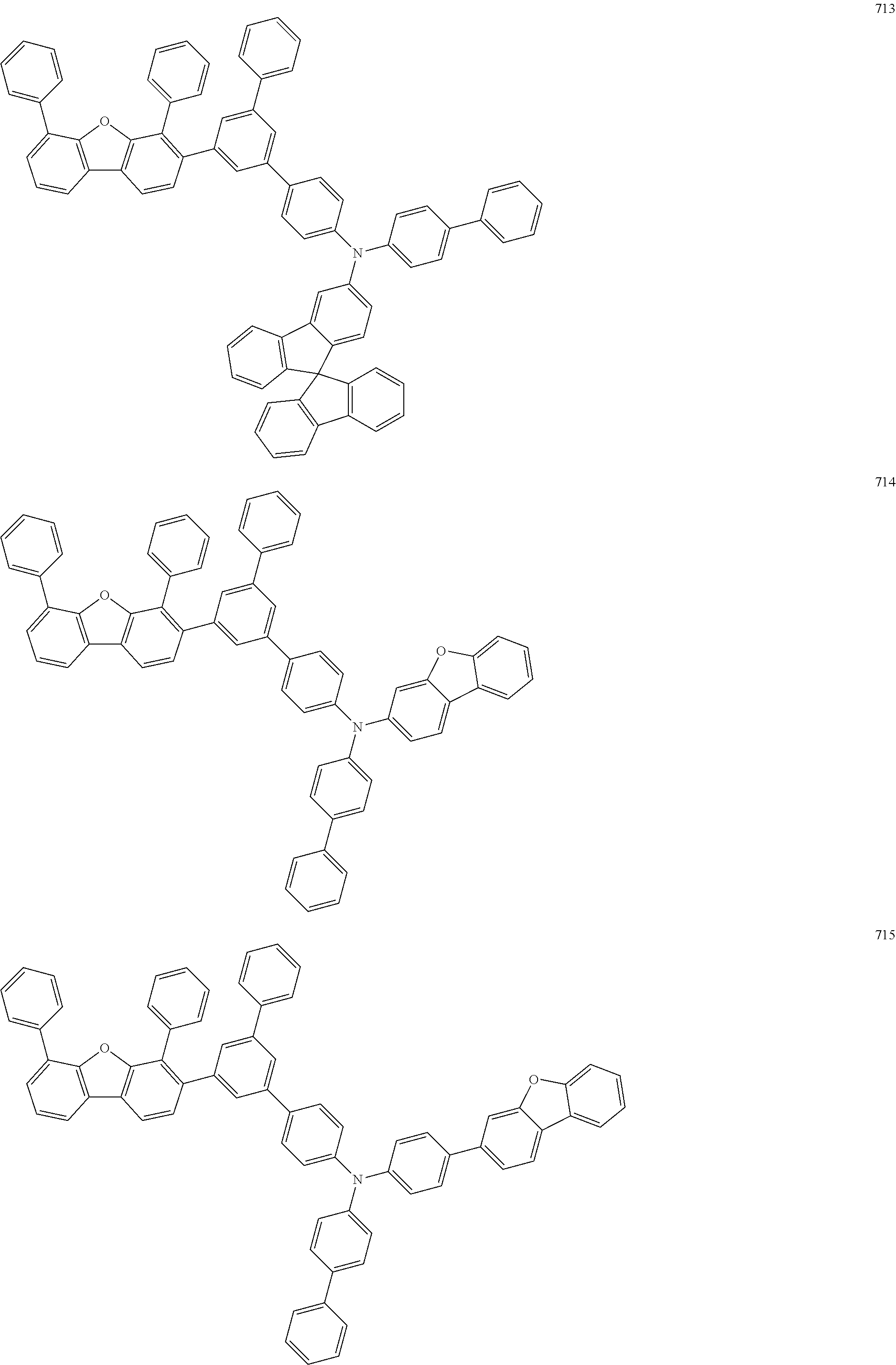

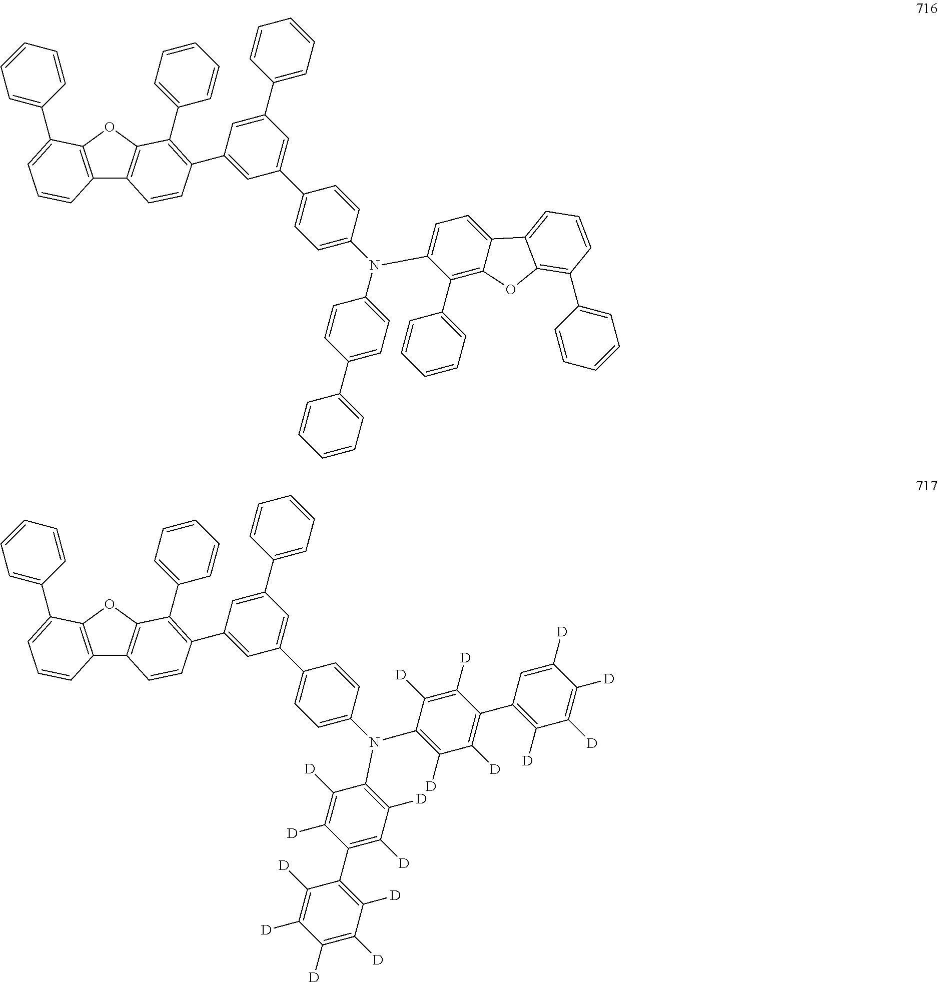 [SMILES: c1ccc(-c2ccc(N(c3ccc(-c4cc(-c5ccccc5)cc(-c5ccc6c(oc7c(-c8ccccc8)cccc76)c5-c5ccccc5)c4)cc3)c3ccc4c(oc5c(-c6ccccc6)cccc54)c3-c3ccccc3)cc2)cc1 ; [2H]c1cc(-c2c([2H])c([2H])c(N(c3ccc(-c4cc(-c5ccccc5)cc(-c5ccc6c(oc7c(-c8ccccc8)cccc76)c5-c5ccccc5)c4)cc3)c3c([2H])c([2H])c(-c4c([2H])c([2H])c([2H])c([2H])c4[2H])c([2H])c3[2H])c([2H])c2[2H])c([2H])c([2H])c1[2H]]

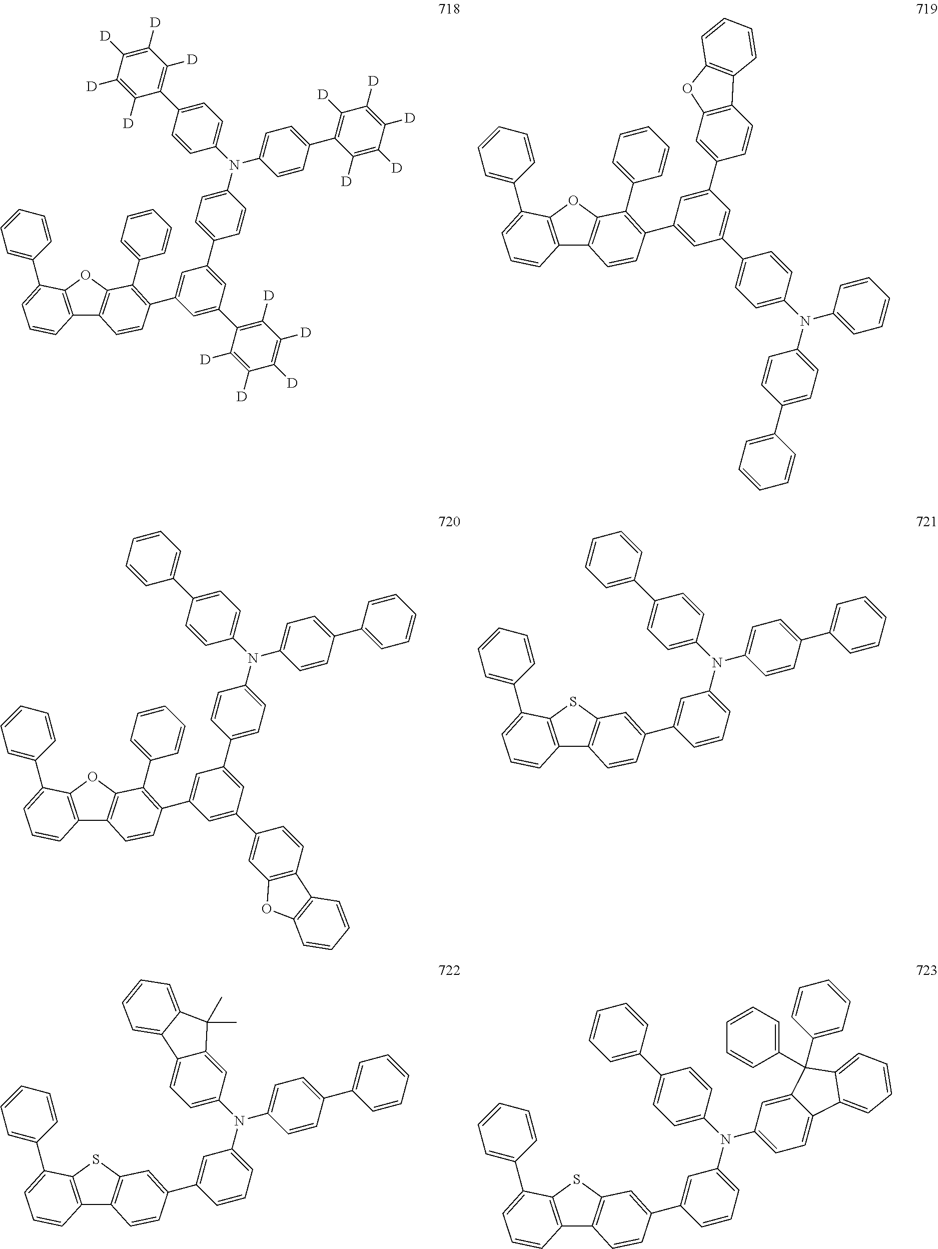

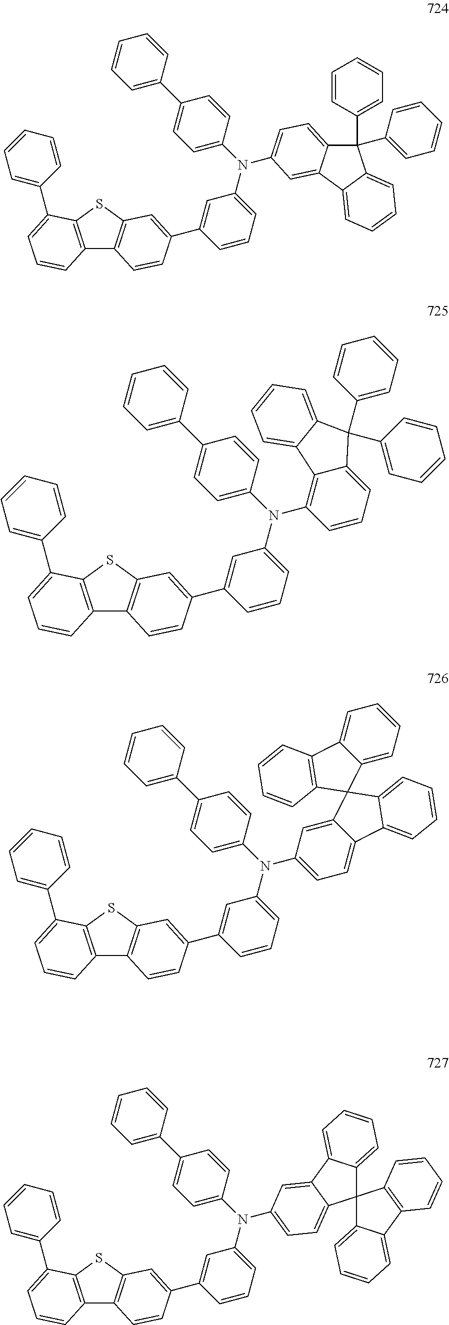 [SMILES: c1ccc(-c2ccc(N(c3cccc(-c4ccc5c(c4)sc4c(-c6ccccc6)cccc45)c3)c3ccc4c(c3)-c3ccccc3C4(c3ccccc3)c3ccccc3)cc2)cc1 ; c1ccc(-c2ccc(N(c3cccc(-c4ccc5c(c4)sc4c(-c6ccccc6)cccc45)c3)c3cccc4c3-c3ccccc3C4(c3ccccc3)c3ccccc3)cc2)cc1 ; c1ccc(-c2ccc(N(c3cccc(-c4ccc5c(c4)sc4c(-c6ccccc6)cccc45)c3)c3ccc4c(c3)C3(c5ccccc5-c5ccccc53)c3ccccc3-4)cc2)cc1 ; c1ccc(-c2ccc(N(c3cccc(-c4ccc5c(c4)sc4c(-c6ccccc6)cccc45)c3)c3ccc4c(c3)-c3ccccc3C43c4ccccc4-c4ccccc43)cc2)cc1]

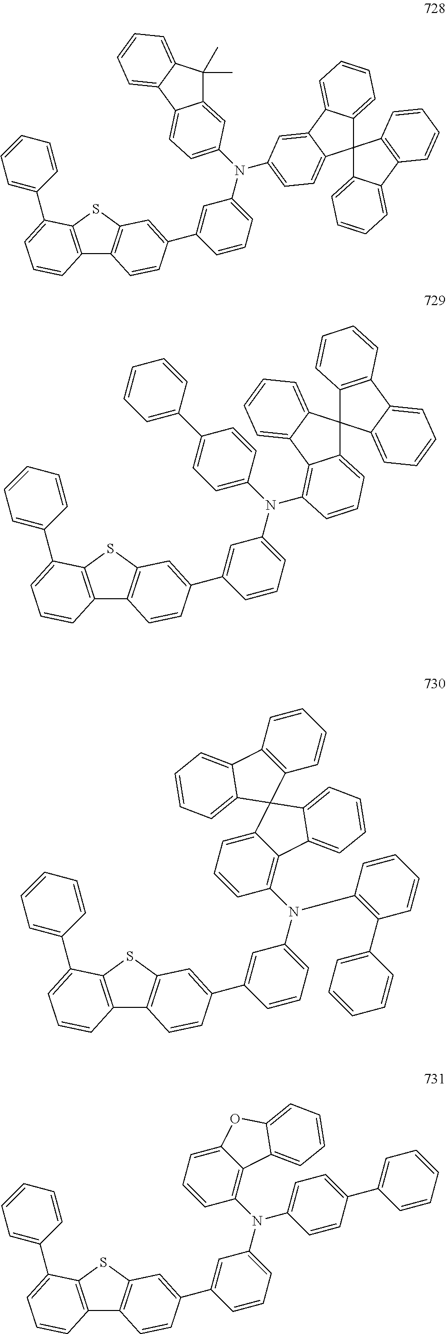 [SMILES: CC1(C)c2ccccc2-c2ccc(N(c3cccc(-c4ccc5c(c4)sc4c(-c6ccccc6)cccc45)c3)c3ccc4c(c3)-c3ccccc3C43c4ccccc4-c4ccccc43)cc21 ; c1ccc(-c2ccc(N(c3cccc(-c4ccc5c(c4)sc4c(-c6ccccc6)cccc45)c3)c3cccc4c3-c3ccccc3C43c4ccccc4-c4ccccc43)cc2)cc1 ; c1ccc(-c2ccccc2N(c2cccc(-c3ccc4c(c3)sc3c(-c5ccccc5)cccc34)c2)c2cccc3c2-c2ccccc2C32c3ccccc3-c3ccccc32)cc1 ; c1ccc(-c2ccc(N(c3cccc(-c4ccc5c(c4)sc4c(-c6ccccc6)cccc45)c3)c3cccc4oc5ccccc5c34)cc2)cc1]

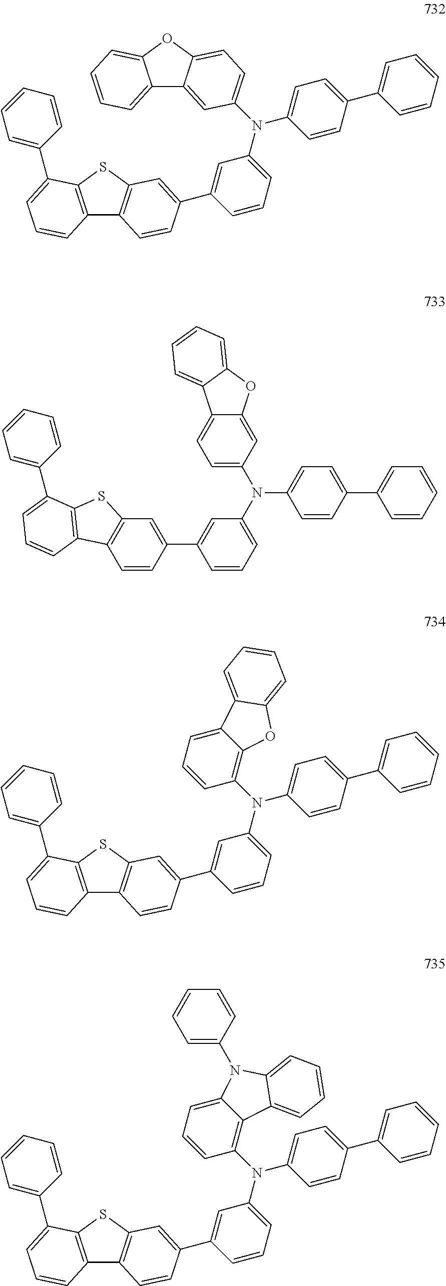 [SMILES: c1ccc(-c2ccc(N(c3cccc(-c4ccc5c(c4)sc4c(-c6ccccc6)cccc45)c3)c3ccc4oc5ccccc5c4c3)cc2)cc1 ; c1ccc(-c2ccc(N(c3cccc(-c4ccc5c(c4)sc4c(-c6ccccc6)cccc45)c3)c3ccc4c(c3)oc3ccccc34)cc2)cc1 ; c1ccc(-c2ccc(N(c3cccc(-c4ccc5c(c4)sc4c(-c6ccccc6)cccc45)c3)c3cccc4c3oc3ccccc34)cc2)cc1 ; c1ccc(-c2ccc(N(c3cccc(-c4ccc5c(c4)sc4c(-c6ccccc6)cccc45)c3)c3cccc4c3c3ccccc3n4-c3ccccc3)cc2)cc1]

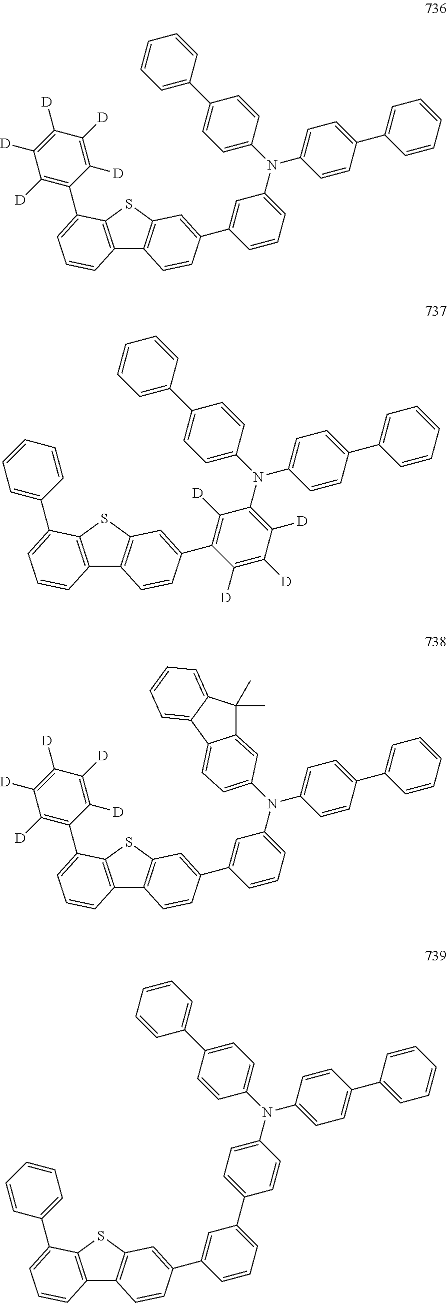 [SMILES: [2H]c1c([2H])c([2H])c(-c2cccc3c2sc2cc(-c4cccc(N(c5ccc(-c6ccccc6)cc5)c5ccc(-c6ccccc6)cc5)c4)ccc23)c([2H])c1[2H] ; [2H]c1c([2H])c(-c2ccc3c(c2)sc2c(-c4ccccc4)cccc23)c([2H])c(N(c2ccc(-c3ccccc3)cc2)c2ccc(-c3ccccc3)cc2)c1[2H] ; [2H]c1c([2H])c([2H])c(-c2cccc3c2sc2cc(-c4cccc(N(c5ccc(-c6ccccc6)cc5)c5ccc6c(c5)C(C)(C)c5ccccc5-6)c4)ccc23)c([2H])c1[2H] ; c1ccc(-c2ccc(N(c3ccc(-c4ccccc4)cc3)c3ccc(-c4cccc(-c5ccc6c(c5)sc5c(-c7ccccc7)cccc56)c4)cc3)cc2)cc1]

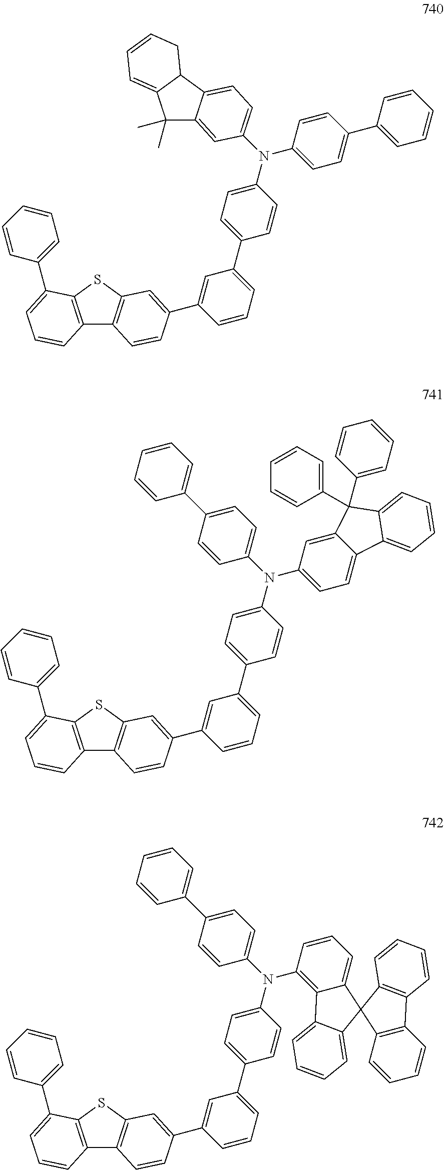 [SMILES: CC1(C)C2=CC=CCC2c2ccc(N(c3ccc(-c4ccccc4)cc3)c3ccc(-c4cccc(-c5ccc6c(c5)sc5c(-c7ccccc7)cccc56)c4)cc3)cc21 ; c1ccc(-c2ccc(N(c3ccc(-c4cccc(-c5ccc6c(c5)sc5c(-c7ccccc7)cccc56)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c(c5)sc5c(-c7ccccc7)cccc56)c4)cc3)c3cccc4c3-c3ccccc3C43c4ccccc4-c4ccccc43)cc2)cc1]

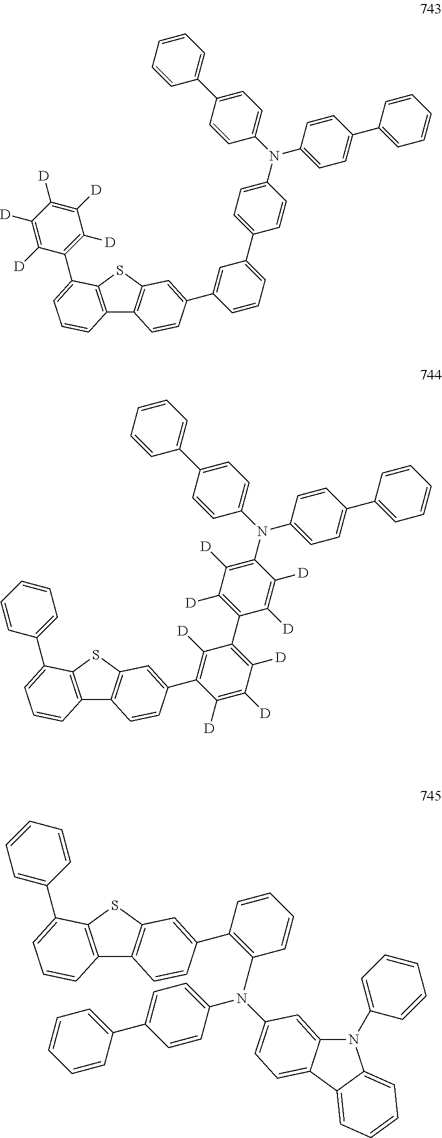 [SMILES: [2H]c1c([2H])c([2H])c(-c2cccc3c2sc2cc(-c4cccc(-c5ccc(N(c6ccc(-c7ccccc7)cc6)c6ccc(-c7ccccc7)cc6)cc5)c4)ccc23)c([2H])c1[2H] ; [2H]c1c([2H])c(-c2ccc3c(c2)sc2c(-c4ccccc4)cccc23)c([2H])c(-c2c([2H])c([2H])c(N(c3ccc(-c4ccccc4)cc3)c3ccc(-c4ccccc4)cc3)c([2H])c2[2H])c1[2H] ; c1ccc(-c2ccc(N(c3ccc4c5ccccc5n(-c5ccccc5)c4c3)c3ccccc3-c3ccc4c(c3)sc3c(-c5ccccc5)cccc34)cc2)cc1]

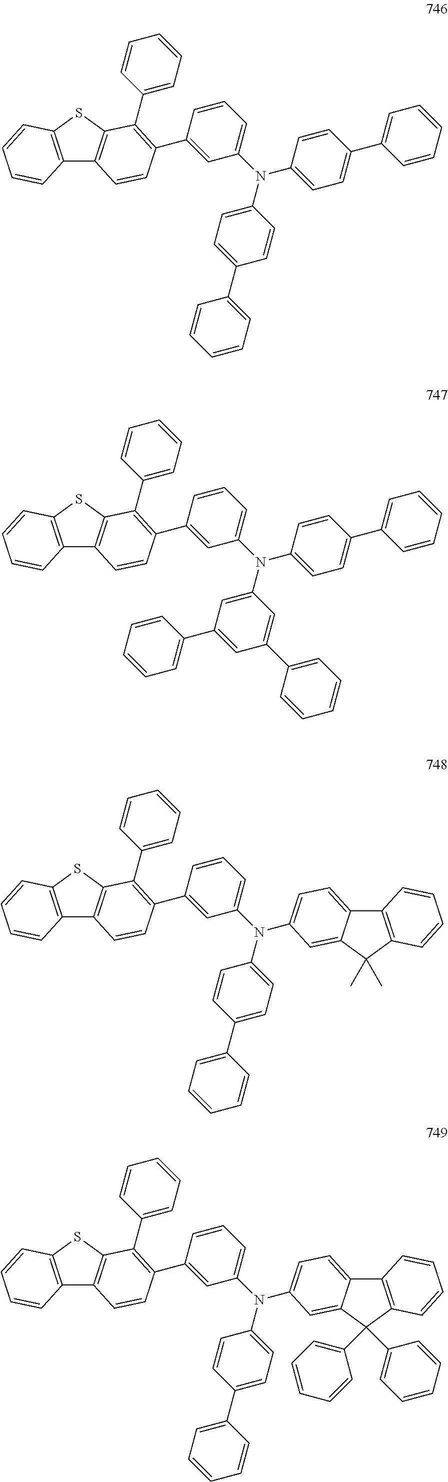 [SMILES: c1ccc(-c2ccc(N(c3ccc(-c4ccccc4)cc3)c3cccc(-c4ccc5c(sc6ccccc65)c4-c4ccccc4)c3)cc2)cc1 ; c1ccc(-c2ccc(N(c3cc(-c4ccccc4)cc(-c4ccccc4)c3)c3cccc(-c4ccc5c(sc6ccccc65)c4-c4ccccc4)c3)cc2)cc1 ; CC1(C)c2ccccc2-c2ccc(N(c3ccc(-c4ccccc4)cc3)c3cccc(-c4ccc5c(sc6ccccc65)c4-c4ccccc4)c3)cc21 ; c1ccc(-c2ccc(N(c3cccc(-c4ccc5c(sc6ccccc65)c4-c4ccccc4)c3)c3ccc4c(c3)C(c3ccccc3)(c3ccccc3)c3ccccc3-4)cc2)cc1]

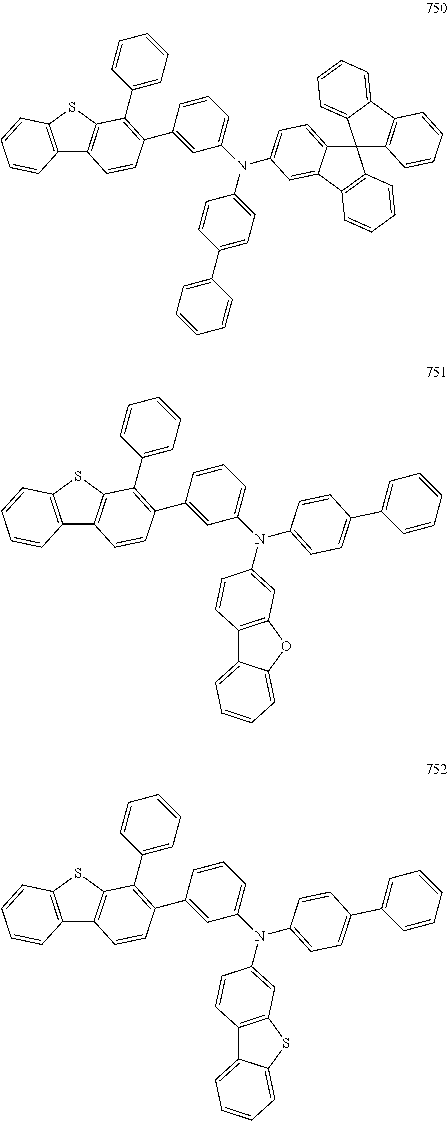 [SMILES: c1ccc(-c2ccc(N(c3cccc(-c4ccc5c(sc6ccccc65)c4-c4ccccc4)c3)c3ccc4c(c3)-c3ccccc3C43c4ccccc4-c4ccccc43)cc2)cc1 ; c1ccc(-c2ccc(N(c3cccc(-c4ccc5c(sc6ccccc65)c4-c4ccccc4)c3)c3ccc4c(c3)oc3ccccc34)cc2)cc1 ; c1ccc(-c2ccc(N(c3cccc(-c4ccc5c(sc6ccccc65)c4-c4ccccc4)c3)c3ccc4c(c3)sc3ccccc34)cc2)cc1]

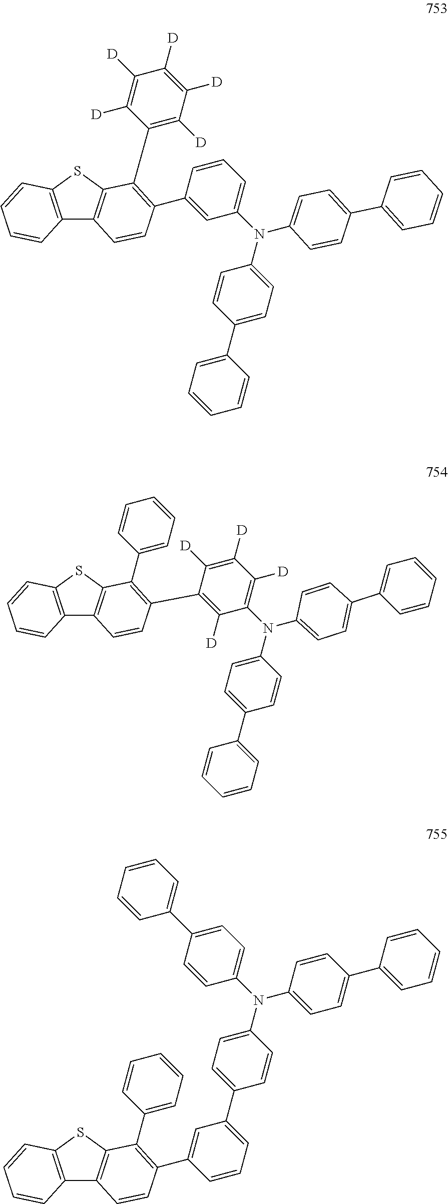 [SMILES: [2H]c1c([2H])c([2H])c(-c2c(-c3cccc(N(c4ccc(-c5ccccc5)cc4)c4ccc(-c5ccccc5)cc4)c3)ccc3c2sc2ccccc23)c([2H])c1[2H] ; [2H]c1c([2H])c(-c2ccc3c(sc4ccccc43)c2-c2ccccc2)c([2H])c(N(c2ccc(-c3ccccc3)cc2)c2ccc(-c3ccccc3)cc2)c1[2H] ; c1ccc(-c2ccc(N(c3ccc(-c4ccccc4)cc3)c3ccc(-c4cccc(-c5ccc6c(sc7ccccc76)c5-c5ccccc5)c4)cc3)cc2)cc1]

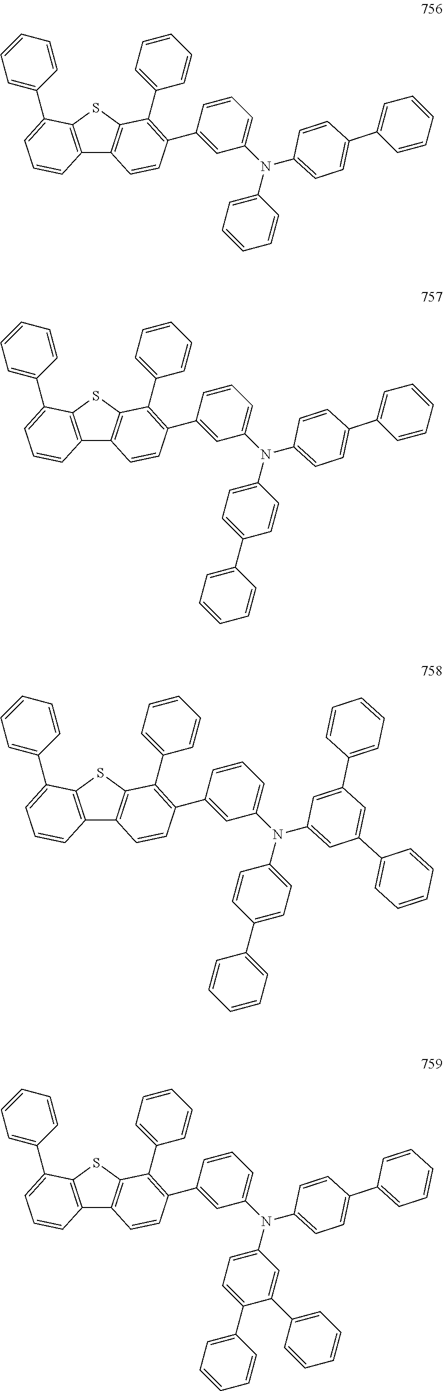

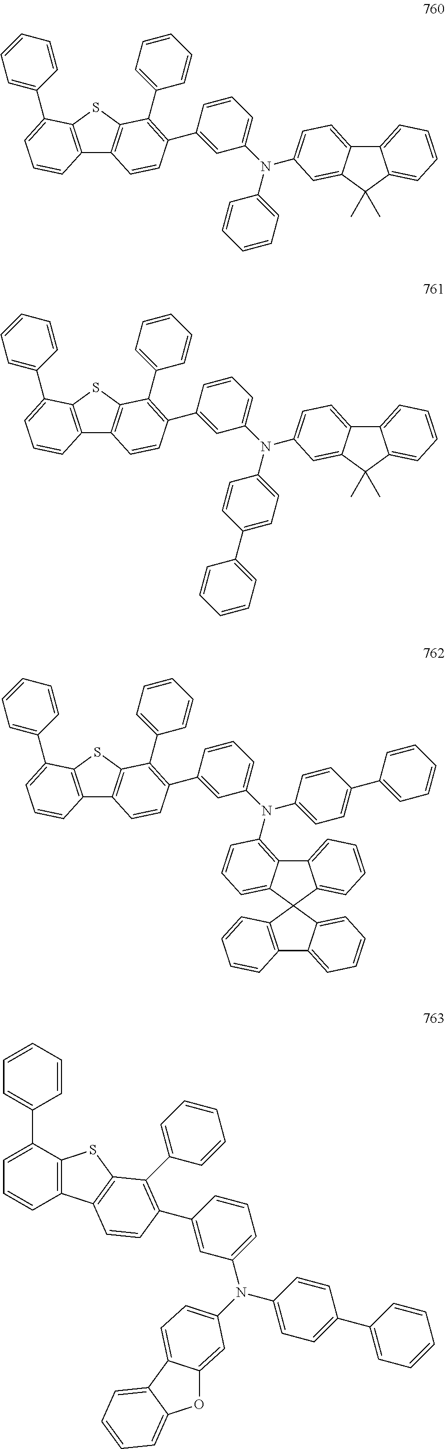 [SMILES: CC1(C)c2ccccc2-c2ccc(N(c3ccccc3)c3cccc(-c4ccc5c(sc6c(-c7ccccc7)cccc65)c4-c4ccccc4)c3)cc21 ; CC1(C)c2ccccc2-c2ccc(N(c3ccc(-c4ccccc4)cc3)c3cccc(-c4ccc5c(sc6c(-c7ccccc7)cccc65)c4-c4ccccc4)c3)cc21 ; c1ccc(-c2ccc(N(c3cccc(-c4ccc5c(sc6c(-c7ccccc7)cccc65)c4-c4ccccc4)c3)c3cccc4c3-c3ccccc3C43c4ccccc4-c4ccccc43)cc2)cc1 ; c1ccc(-c2ccc(N(c3cccc(-c4ccc5c(sc6c(-c7ccccc7)cccc65)c4-c4ccccc4)c3)c3ccc4c(c3)oc3ccccc34)cc2)cc1]

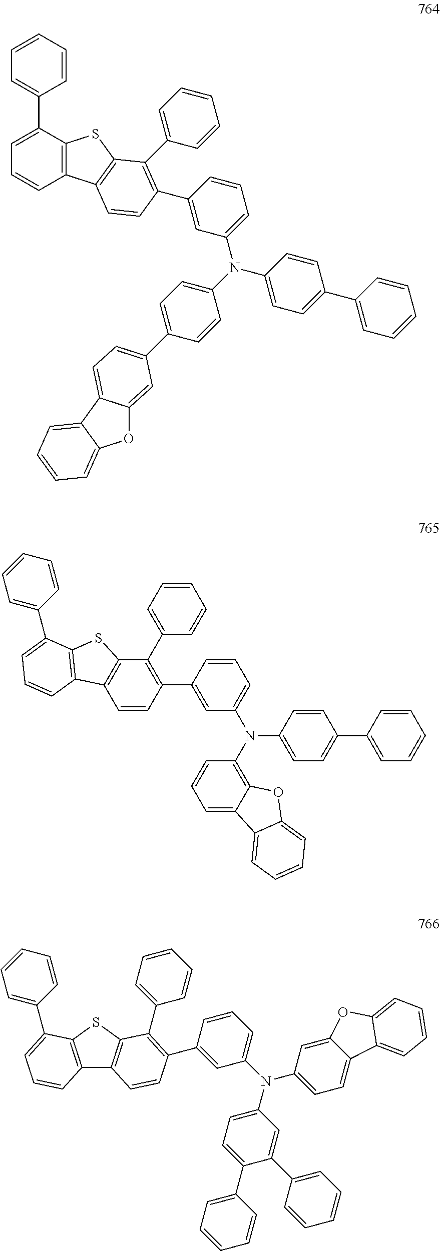 [SMILES: c1ccc(-c2ccc(N(c3ccc(-c4ccc5c(c4)oc4ccccc45)cc3)c3cccc(-c4ccc5c(sc6c(-c7ccccc7)cccc65)c4-c4ccccc4)c3)cc2)cc1 ; c1ccc(-c2ccc(N(c3cccc(-c4ccc5c(sc6c(-c7ccccc7)cccc65)c4-c4ccccc4)c3)c3cccc4c3oc3ccccc34)cc2)cc1 ; c1ccc(-c2ccc(N(c3cccc(-c4ccc5c(sc6c(-c7ccccc7)cccc65)c4-c4ccccc4)c3)c3ccc4c(c3)oc3ccccc34)cc2-c2ccccc2)cc1]

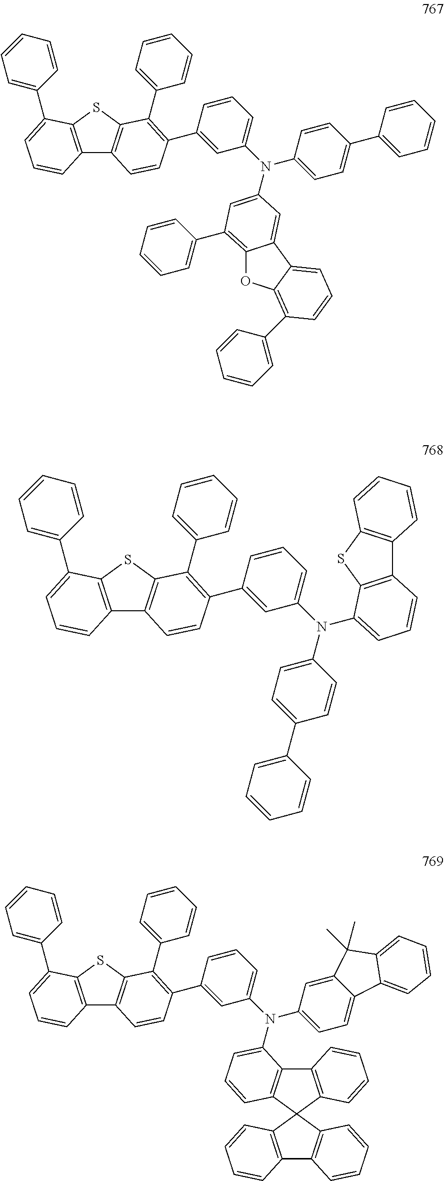 [SMILES: c1ccc(-c2ccc(N(c3cccc(-c4ccc5c(sc6c(-c7ccccc7)cccc65)c4-c4ccccc4)c3)c3cc(-c4ccccc4)c4oc5c(-c6ccccc6)cccc5c4c3)cc2)cc1 ; c1ccc(-c2ccc(N(c3cccc(-c4ccc5c(sc6c(-c7ccccc7)cccc65)c4-c4ccccc4)c3)c3cccc4c3sc3ccccc34)cc2)cc1 ; CC1(C)c2ccccc2-c2ccc(N(c3cccc(-c4ccc5c(sc6c(-c7ccccc7)cccc65)c4-c4ccccc4)c3)c3cccc4c3-c3ccccc3C43c4ccccc4-c4ccccc43)cc21]

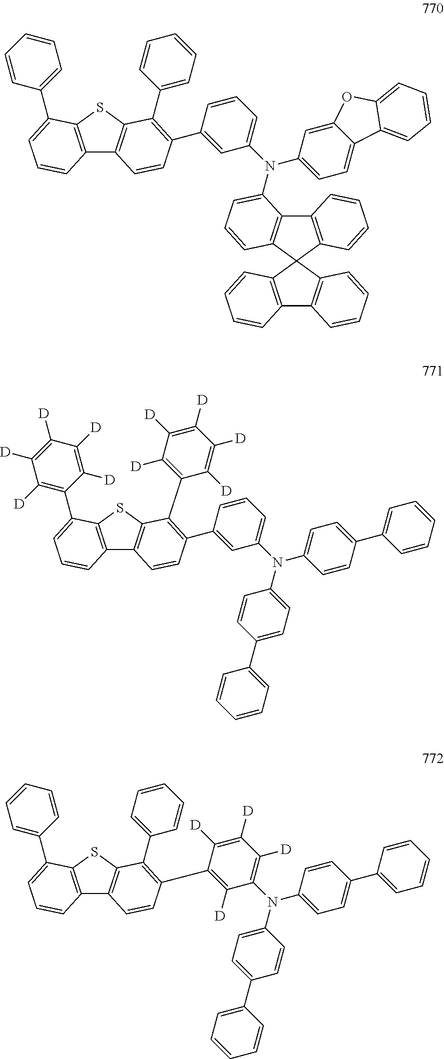 [SMILES: c1ccc(-c2cccc3c2sc2c(-c4ccccc4)c(-c4cccc(N(c5ccc6c(c5)oc5ccccc56)c5cccc6c5-c5ccccc5C65c6ccccc6-c6ccccc65)c4)ccc23)cc1 ; [2H]c1c([2H])c([2H])c(-c2cccc3c2sc2c(-c4c([2H])c([2H])c([2H])c([2H])c4[2H])c(-c4cccc(N(c5ccc(-c6ccccc6)cc5)c5ccc(-c6ccccc6)cc5)c4)ccc23)c([2H])c1[2H] ; [2H]c1c([2H])c(-c2ccc3c(sc4c(-c5ccccc5)cccc43)c2-c2ccccc2)c([2H])c(N(c2ccc(-c3ccccc3)cc2)c2ccc(-c3ccccc3)cc2)c1[2H]]

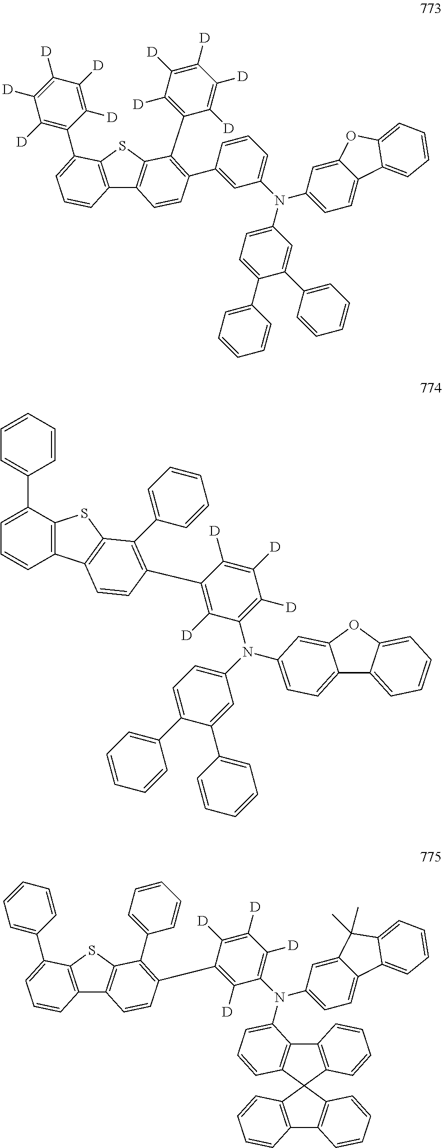 [SMILES: [2H]c1c([2H])c([2H])c(-c2cccc3c2sc2c(-c4c([2H])c([2H])c([2H])c([2H])c4[2H])c(-c4cccc(N(c5ccc(-c6ccccc6)c(-c6ccccc6)c5)c5ccc6c(c5)oc5ccccc56)c4)ccc23)c([2H])c1[2H] ; [2H]c1c([2H])c(-c2ccc3c(sc4c(-c5ccccc5)cccc43)c2-c2ccccc2)c([2H])c(N(c2ccc(-c3ccccc3)c(-c3ccccc3)c2)c2ccc3c(c2)oc2ccccc23)c1[2H] ; [2H]c1c([2H])c(-c2ccc3c(sc4c(-c5ccccc5)cccc43)c2-c2ccccc2)c([2H])c(N(c2ccc3c(c2)C(C)(C)c2ccccc2-3)c2cccc3c2-c2ccccc2C32c3ccccc3-c3ccccc32)c1[2H]]

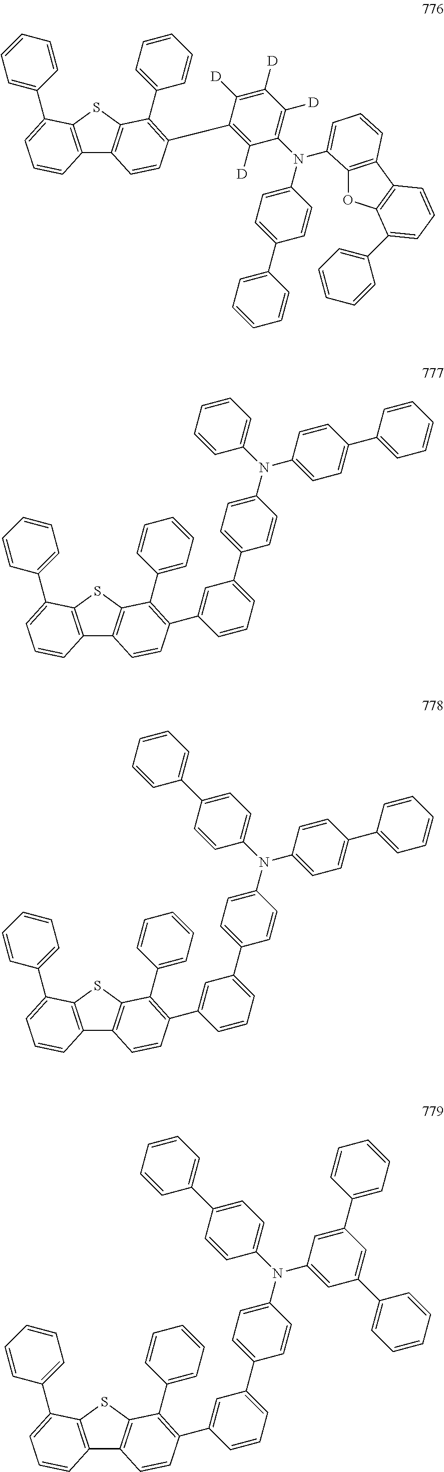 [SMILES: [2H]c1c([2H])c(-c2ccc3c(sc4c(-c5ccccc5)cccc43)c2-c2ccccc2)c([2H])c(N(c2ccc(-c3ccccc3)cc2)c2cccc3c2oc2c(-c4ccccc4)cccc23)c1[2H] ; c1ccc(-c2ccc(N(c3ccccc3)c3ccc(-c4cccc(-c5ccc6c(sc7c(-c8ccccc8)cccc76)c5-c5ccccc5)c4)cc3)cc2)cc1 ; c1ccc(-c2ccc(N(c3ccc(-c4ccccc4)cc3)c3ccc(-c4cccc(-c5ccc6c(sc7c(-c8ccccc8)cccc76)c5-c5ccccc5)c4)cc3)cc2)cc1 ; c1ccc(-c2ccc(N(c3ccc(-c4cccc(-c5ccc6c(sc7c(-c8ccccc8)cccc76)c5-c5ccccc5)c4)cc3)c3cc(-c4ccccc4)cc(-c4ccccc4)c3)cc2)cc1]

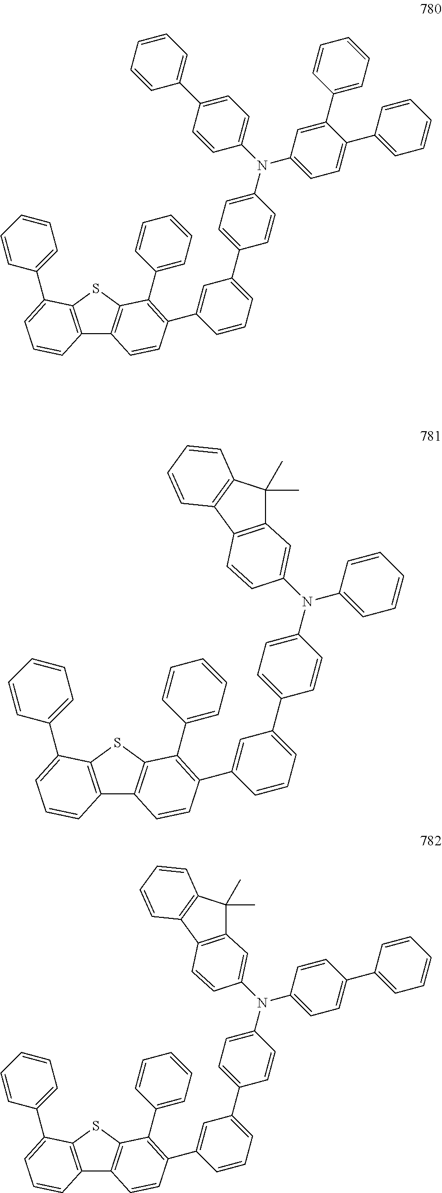 [SMILES: c1ccc(-c2ccc(N(c3ccc(-c4cccc(-c5ccc6c(sc7c(-c8ccccc8)cccc76)c5-c5ccccc5)c4)cc3)c3ccc(-c4ccccc4)c(-c4ccccc4)c3)cc2)cc1 ; CC1(C)c2ccccc2-c2ccc(N(c3ccccc3)c3ccc(-c4cccc(-c5ccc6c(sc7c(-c8ccccc8)cccc76)c5-c5ccccc5)c4)cc3)cc21 ; CC1(C)c2ccccc2-c2ccc(N(c3ccc(-c4ccccc4)cc3)c3ccc(-c4cccc(-c5ccc6c(sc7c(-c8ccccc8)cccc76)c5-c5ccccc5)c4)cc3)cc21]

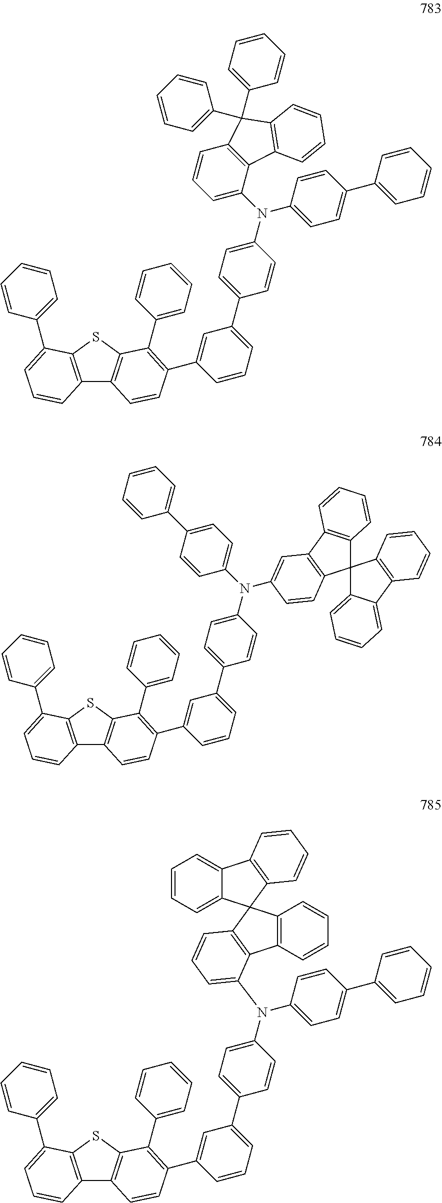 [SMILES: c1ccc(-c2ccc(N(c3ccc(-c4cccc(-c5ccc6c(sc7c(-c8ccccc8)cccc76)c5-c5ccccc5)c4)cc3)c3cccc4c3-c3ccccc3C4(c3ccccc3)c3ccccc3)cc2)cc1 ; c1ccc(-c2ccc(N(c3ccc(-c4cccc(-c5ccc6c(sc7c(-c8ccccc8)cccc76)c5-c5ccccc5)c4)cc3)c3ccc4c(c3)-c3ccccc3C43c4ccccc4-c4ccccc43)cc2)cc1 ; c1ccc(-c2ccc(N(c3ccc(-c4cccc(-c5ccc6c(sc7c(-c8ccccc8)cccc76)c5-c5ccccc5)c4)cc3)c3cccc4c3-c3ccccc3C43c4ccccc4-c4ccccc43)cc2)cc1]

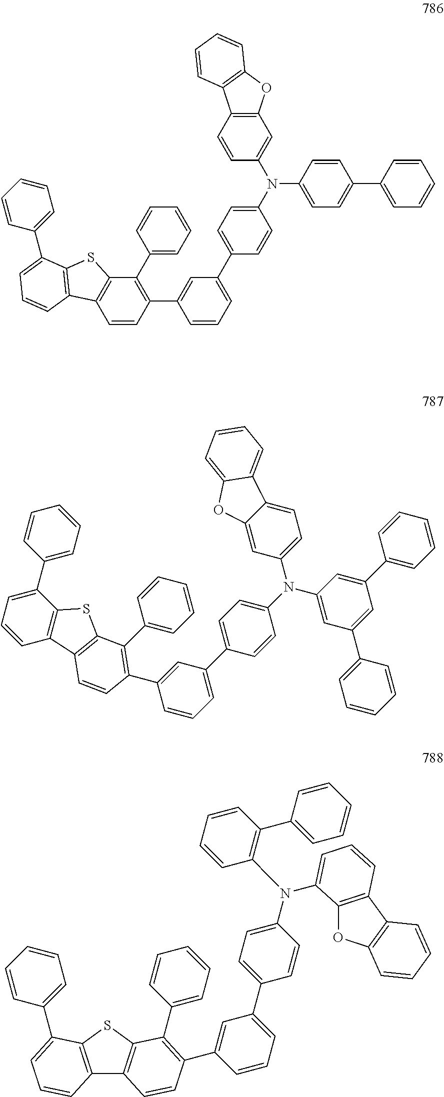 [SMILES: c1ccc(-c2ccc(N(c3ccc(-c4cccc(-c5ccc6c(sc7c(-c8ccccc8)cccc76)c5-c5ccccc5)c4)cc3)c3ccc4c(c3)oc3ccccc34)cc2)cc1 ; c1ccc(-c2cc(-c3ccccc3)cc(N(c3ccc(-c4cccc(-c5ccc6c(sc7c(-c8ccccc8)cccc76)c5-c5ccccc5)c4)cc3)c3ccc4c(c3)oc3ccccc34)c2)cc1 ; c1ccc(-c2ccccc2N(c2ccc(-c3cccc(-c4ccc5c(sc6c(-c7ccccc7)cccc65)c4-c4ccccc4)c3)cc2)c2cccc3c2oc2ccccc23)cc1]

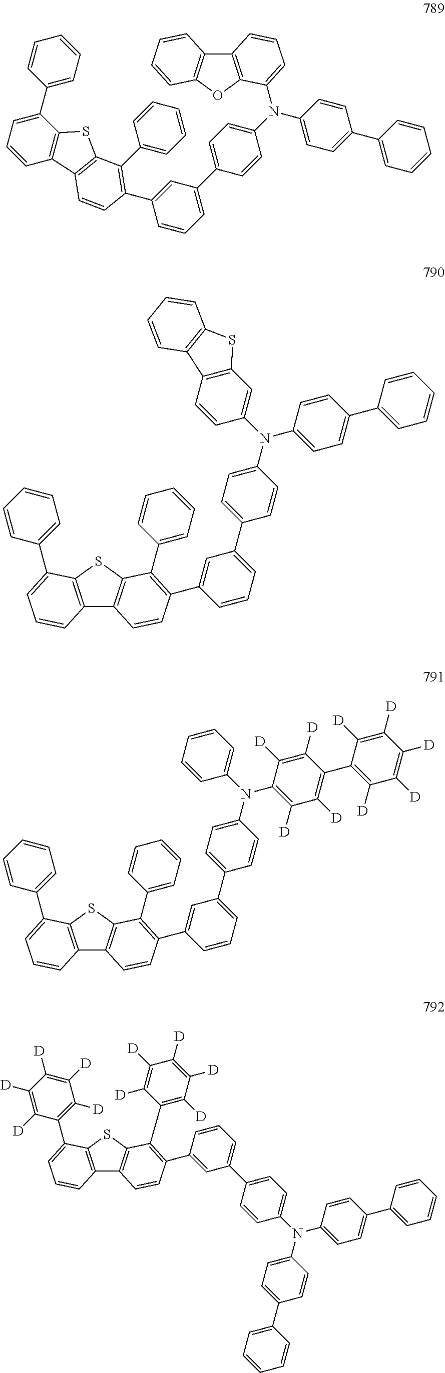

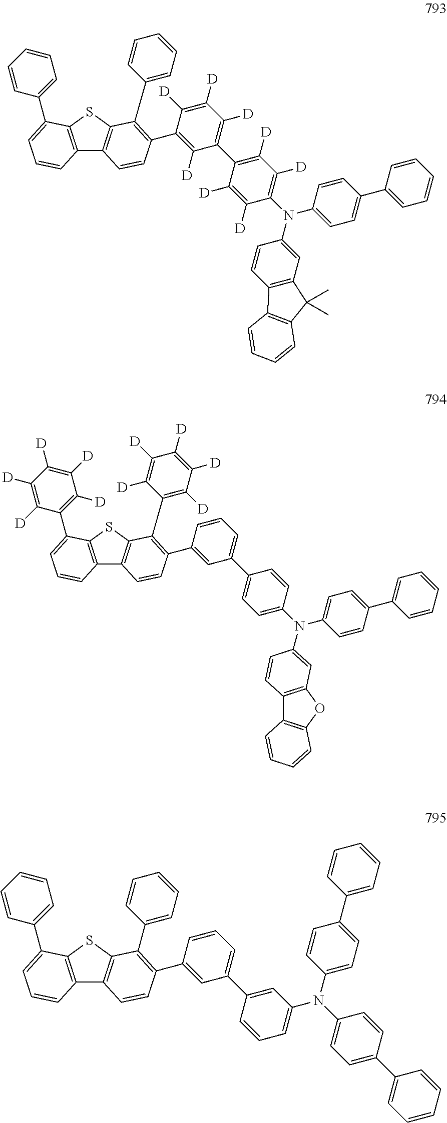 [SMILES: [2H]c1c([2H])c(-c2ccc3c(sc4c(-c5ccccc5)cccc43)c2-c2ccccc2)c([2H])c(-c2c([2H])c([2H])c(N(c3ccc(-c4ccccc4)cc3)c3ccc4c(c3)C(C)(C)c3ccccc3-4)c([2H])c2[2H])c1[2H] ; [2H]c1c([2H])c([2H])c(-c2cccc3c2sc2c(-c4c([2H])c([2H])c([2H])c([2H])c4[2H])c(-c4cccc(-c5ccc(N(c6ccc(-c7ccccc7)cc6)c6ccc7c(c6)oc6ccccc67)cc5)c4)ccc23)c([2H])c1[2H] ; c1ccc(-c2ccc(N(c3ccc(-c4ccccc4)cc3)c3cccc(-c4cccc(-c5ccc6c(sc7c(-c8ccccc8)cccc76)c5-c5ccccc5)c4)c3)cc2)cc1]

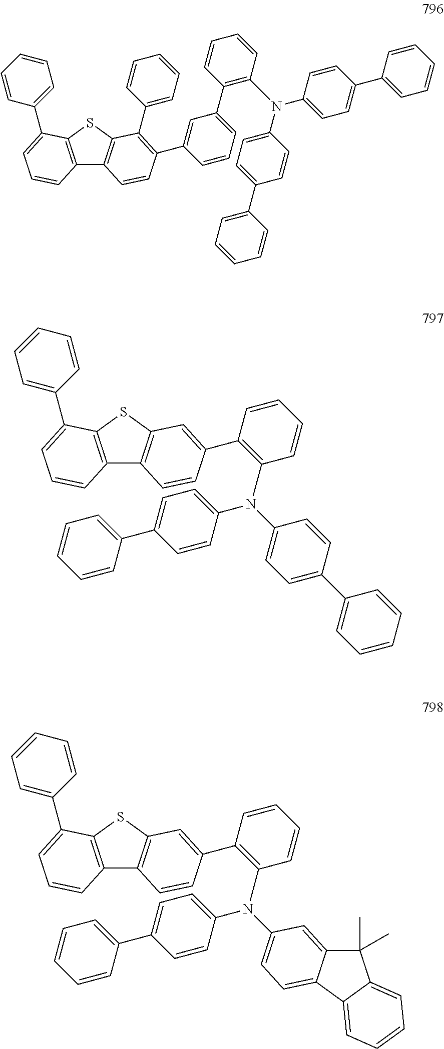 [SMILES: c1ccc(-c2ccc(N(c3ccc(-c4ccccc4)cc3)c3ccccc3-c3cccc(-c4ccc5c(sc6c(-c7ccccc7)cccc65)c4-c4ccccc4)c3)cc2)cc1 ; c1ccc(-c2ccc(N(c3ccc(-c4ccccc4)cc3)c3ccccc3-c3ccc4c(c3)sc3c(-c5ccccc5)cccc34)cc2)cc1 ; CC1(C)c2ccccc2-c2ccc(N(c3ccc(-c4ccccc4)cc3)c3ccccc3-c3ccc4c(c3)sc3c(-c5ccccc5)cccc34)cc21]

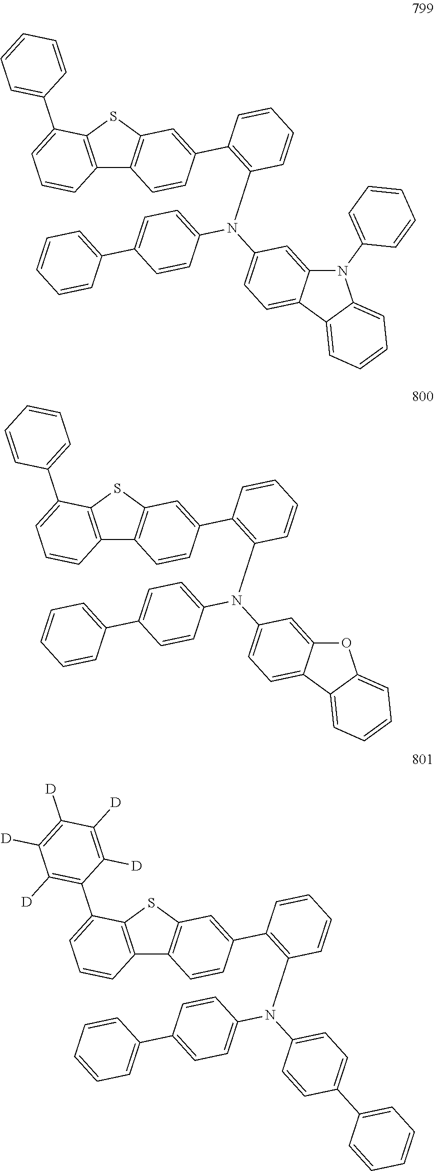 [SMILES: c1ccc(-c2ccc(N(c3ccc4c5ccccc5n(-c5ccccc5)c4c3)c3ccccc3-c3ccc4c(c3)sc3c(-c5ccccc5)cccc34)cc2)cc1 ; c1ccc(-c2ccc(N(c3ccc4c(c3)oc3ccccc34)c3ccccc3-c3ccc4c(c3)sc3c(-c5ccccc5)cccc34)cc2)cc1 ; [2H]c1c([2H])c([2H])c(-c2cccc3c2sc2cc(-c4ccccc4N(c4ccc(-c5ccccc5)cc4)c4ccc(-c5ccccc5)cc4)ccc23)c([2H])c1[2H]]

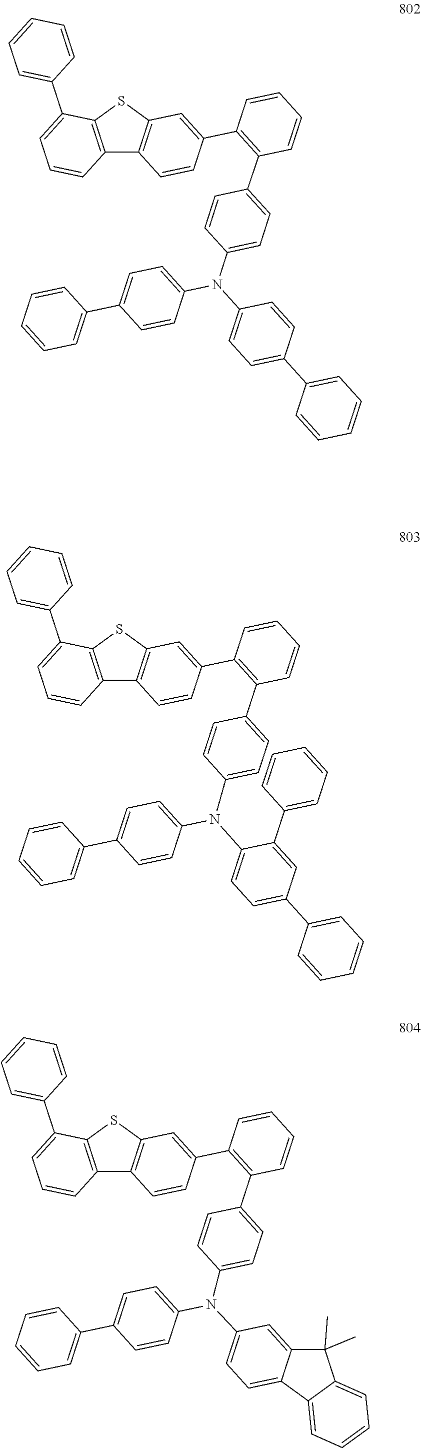 [SMILES: c1ccc(-c2ccc(N(c3ccc(-c4ccccc4)cc3)c3ccc(-c4ccccc4-c4ccc5c(c4)sc4c(-c6ccccc6)cccc45)cc3)cc2)cc1 ; c1ccc(-c2ccc(N(c3ccc(-c4ccccc4-c4ccc5c(c4)sc4c(-c6ccccc6)cccc45)cc3)c3ccc(-c4ccccc4)cc3-c3ccccc3)cc2)cc1 ; CC1(C)c2ccccc2-c2ccc(N(c3ccc(-c4ccccc4)cc3)c3ccc(-c4ccccc4-c4ccc5c(c4)sc4c(-c6ccccc6)cccc45)cc3)cc21]

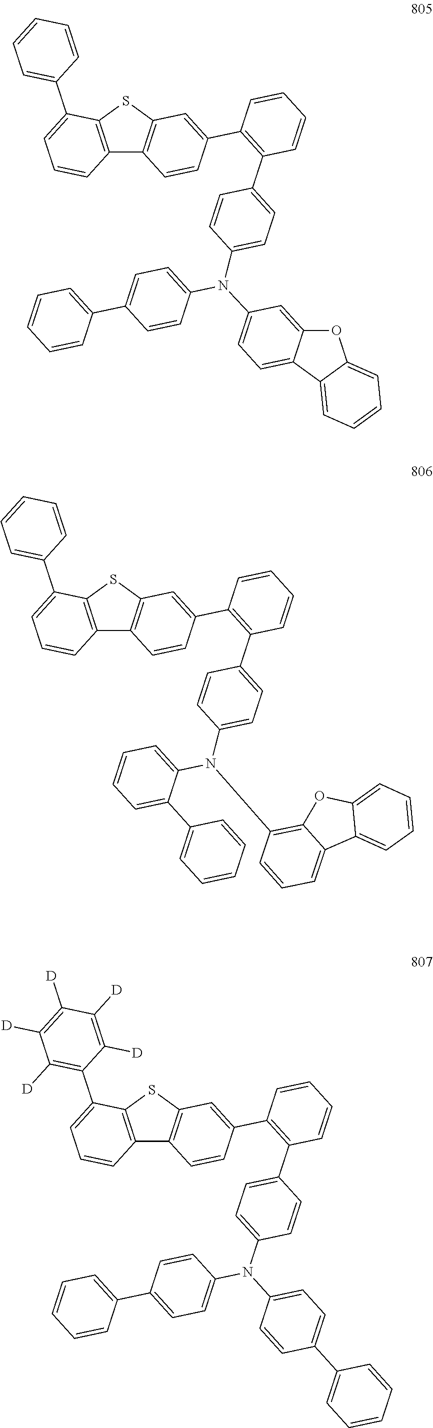 [SMILES: c1ccc(-c2ccc(N(c3ccc(-c4ccccc4-c4ccc5c(c4)sc4c(-c6ccccc6)cccc45)cc3)c3ccc4c(c3)oc3ccccc34)cc2)cc1 ; c1ccc(-c2ccccc2N(c2ccc(-c3ccccc3-c3ccc4c(c3)sc3c(-c5ccccc5)cccc34)cc2)c2cccc3c2oc2ccccc23)cc1 ; [2H]c1c([2H])c([2H])c(-c2cccc3c2sc2cc(-c4ccccc4-c4ccc(N(c5ccc(-c6ccccc6)cc5)c5ccc(-c6ccccc6)cc5)cc4)ccc23)c([2H])c1[2H]]

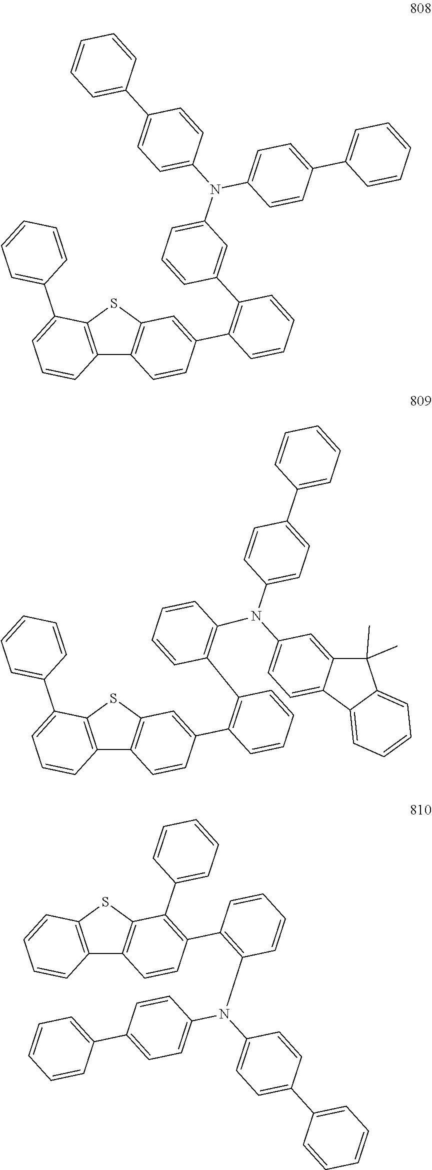 [SMILES: c1ccc(-c2ccc(N(c3ccc(-c4ccccc4)cc3)c3cccc(-c4ccccc4-c4ccc5c(c4)sc4c(-c6ccccc6)cccc45)c3)cc2)cc1 ; CC1(C)c2ccccc2-c2ccc(N(c3ccc(-c4ccccc4)cc3)c3ccccc3-c3ccccc3-c3ccc4c(c3)sc3c(-c5ccccc5)cccc34)cc21 ; c1ccc(-c2ccc(N(c3ccc(-c4ccccc4)cc3)c3ccccc3-c3ccc4c(sc5ccccc54)c3-c3ccccc3)cc2)cc1]

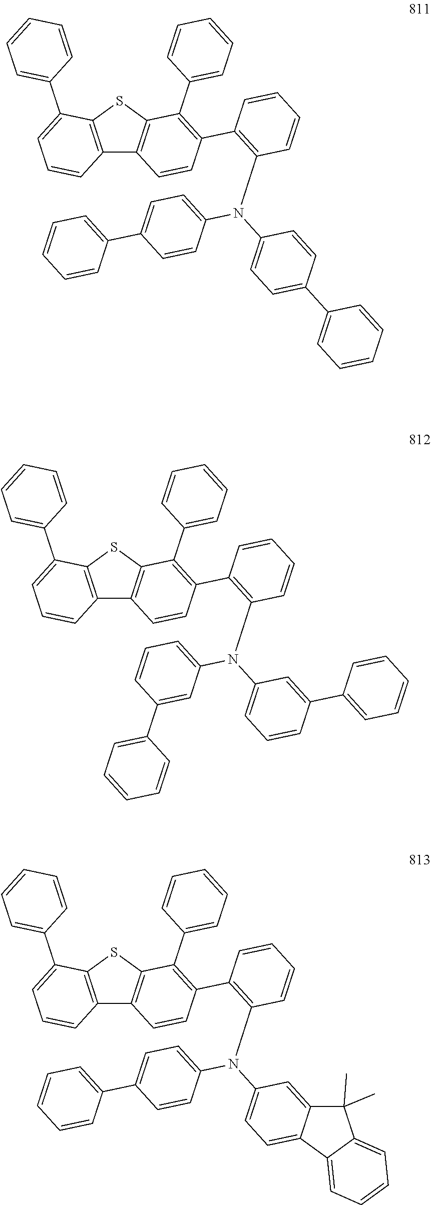 [SMILES: c1ccc(-c2ccc(N(c3ccc(-c4ccccc4)cc3)c3ccccc3-c3ccc4c(sc5c(-c6ccccc6)cccc54)c3-c3ccccc3)cc2)cc1 ; c1ccc(-c2cccc(N(c3cccc(-c4ccccc4)c3)c3ccccc3-c3ccc4c(sc5c(-c6ccccc6)cccc54)c3-c3ccccc3)c2)cc1 ; CC1(C)c2ccccc2-c2ccc(N(c3ccc(-c4ccccc4)cc3)c3ccccc3-c3ccc4c(sc5c(-c6ccccc6)cccc54)c3-c3ccccc3)cc21]

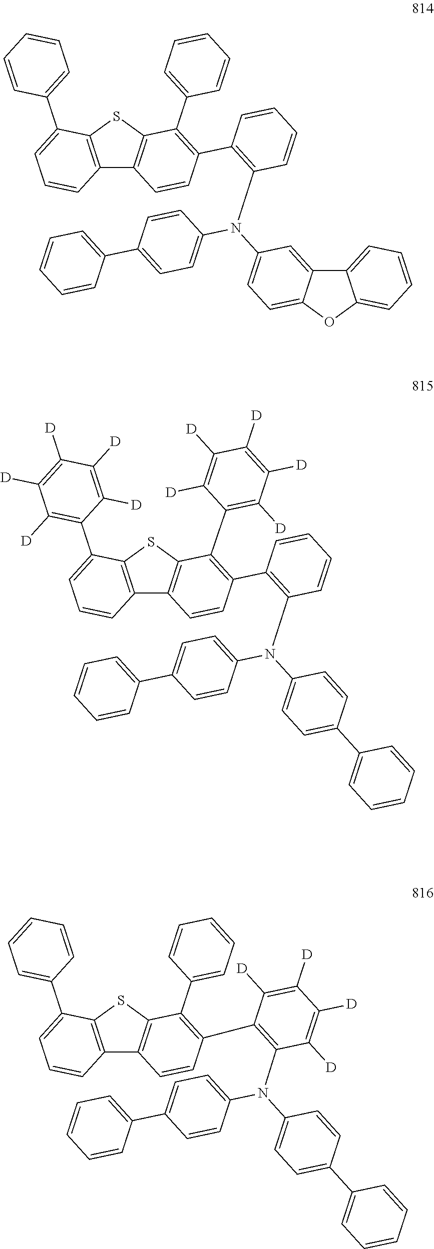 [SMILES: c1ccc(-c2ccc(N(c3ccc4oc5ccccc5c4c3)c3ccccc3-c3ccc4c(sc5c(-c6ccccc6)cccc54)c3-c3ccccc3)cc2)cc1 ; [2H]c1c([2H])c([2H])c(-c2cccc3c2sc2c(-c4c([2H])c([2H])c([2H])c([2H])c4[2H])c(-c4ccccc4N(c4ccc(-c5ccccc5)cc4)c4ccc(-c5ccccc5)cc4)ccc23)c([2H])c1[2H] ; [2H]c1c([2H])c([2H])c(N(c2ccc(-c3ccccc3)cc2)c2ccc(-c3ccccc3)cc2)c(-c2ccc3c(sc4c(-c5ccccc5)cccc43)c2-c2ccccc2)c1[2H]]

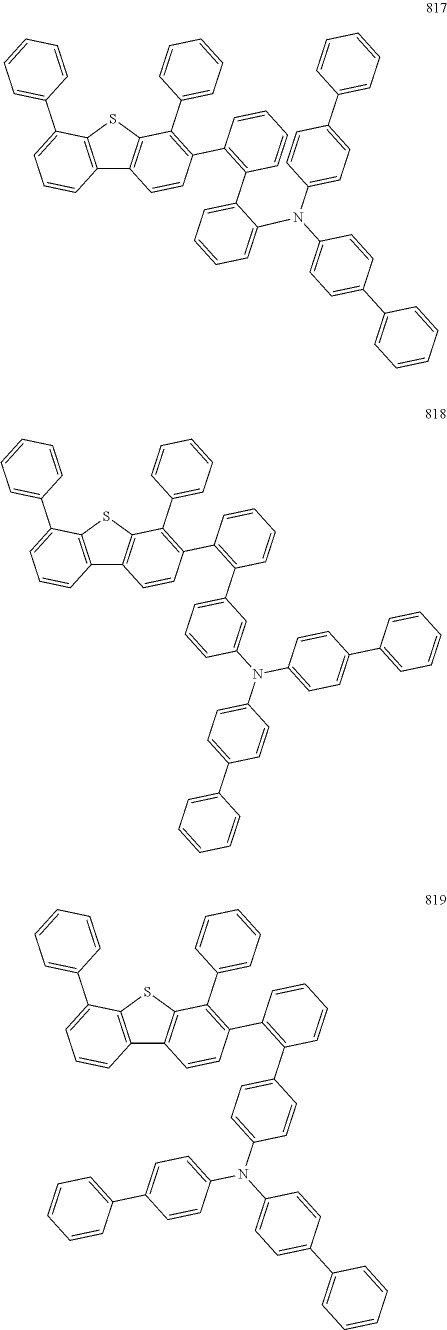 [SMILES: c1ccc(-c2ccc(N(c3ccc(-c4ccccc4)cc3)c3ccccc3-c3ccccc3-c3ccc4c(sc5c(-c6ccccc6)cccc54)c3-c3ccccc3)cc2)cc1 ; c1ccc(-c2ccc(N(c3ccc(-c4ccccc4)cc3)c3cccc(-c4ccccc4-c4ccc5c(sc6c(-c7ccccc7)cccc65)c4-c4ccccc4)c3)cc2)cc1 ; c1ccc(-c2ccc(N(c3ccc(-c4ccccc4)cc3)c3ccc(-c4ccccc4-c4ccc5c(sc6c(-c7ccccc7)cccc65)c4-c4ccccc4)cc3)cc2)cc1]

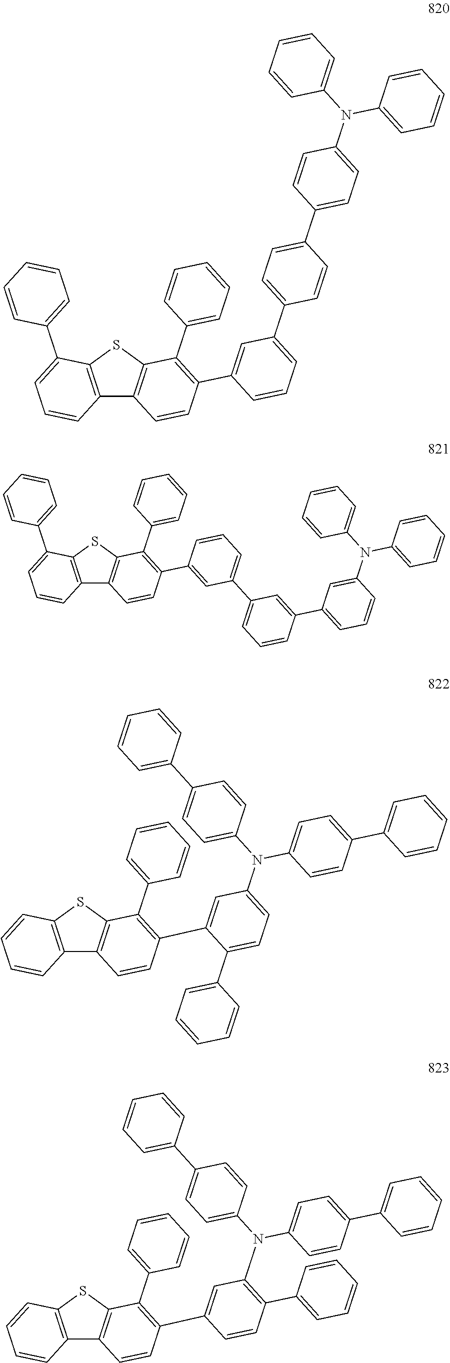 [SMILES: c1ccc(-c2cccc3c2sc2c(-c4ccccc4)c(-c4cccc(-c5ccc(-c6ccc(N(c7ccccc7)c7ccccc7)cc6)cc5)c4)ccc23)cc1 ; c1ccc(-c2cccc3c2sc2c(-c4ccccc4)c(-c4cccc(-c5cccc(-c6cccc(N(c7ccccc7)c7ccccc7)c6)c5)c4)ccc23)cc1 ; c1ccc(-c2ccc(N(c3ccc(-c4ccccc4)cc3)c3ccc(-c4ccccc4)c(-c4ccc5c(sc6ccccc65)c4-c4ccccc4)c3)cc2)cc1 ; c1ccc(-c2ccc(N(c3ccc(-c4ccccc4)cc3)c3cc(-c4ccc5c(sc6ccccc65)c4-c4ccccc4)ccc3-c3ccccc3)cc2)cc1]

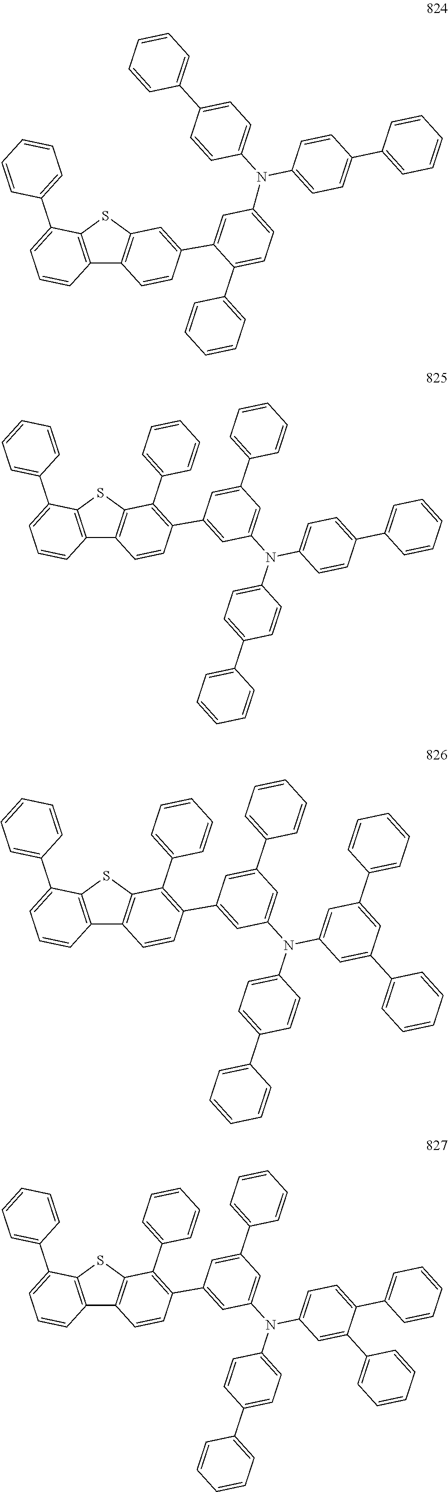 [SMILES: c1ccc(-c2ccc(N(c3ccc(-c4ccccc4)cc3)c3ccc(-c4ccccc4)c(-c4ccc5c(c4)sc4c(-c6ccccc6)cccc45)c3)cc2)cc1 ; c1ccc(-c2ccc(N(c3ccc(-c4ccccc4)cc3)c3cc(-c4ccccc4)cc(-c4ccc5c(sc6c(-c7ccccc7)cccc65)c4-c4ccccc4)c3)cc2)cc1 ; c1ccc(-c2ccc(N(c3cc(-c4ccccc4)cc(-c4ccccc4)c3)c3cc(-c4ccccc4)cc(-c4ccc5c(sc6c(-c7ccccc7)cccc65)c4-c4ccccc4)c3)cc2)cc1 ; c1ccc(-c2ccc(N(c3cc(-c4ccccc4)cc(-c4ccc5c(sc6c(-c7ccccc7)cccc65)c4-c4ccccc4)c3)c3ccc(-c4ccccc4)c(-c4ccccc4)c3)cc2)cc1]

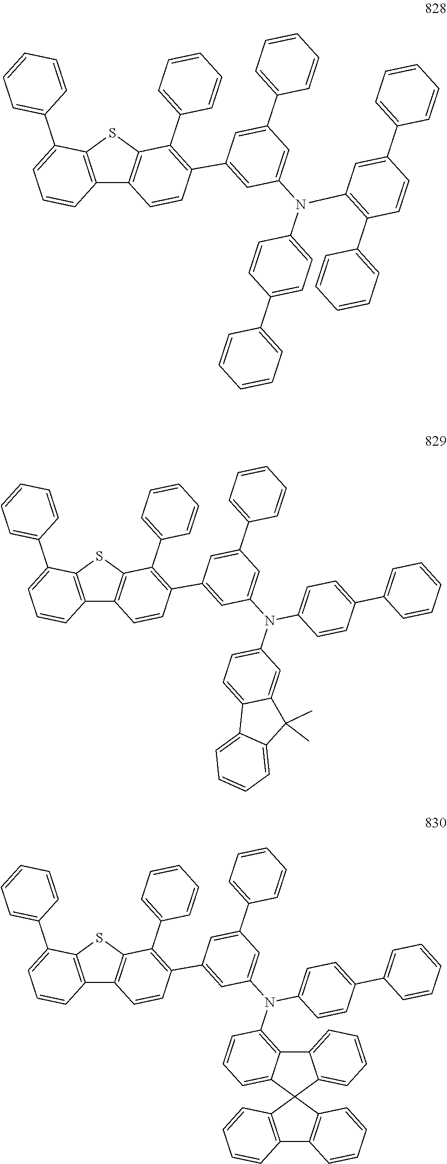 [SMILES: c1ccc(-c2ccc(N(c3cc(-c4ccccc4)cc(-c4ccc5c(sc6c(-c7ccccc7)cccc65)c4-c4ccccc4)c3)c3cc(-c4ccccc4)ccc3-c3ccccc3)cc2)cc1 ; CC1(C)c2ccccc2-c2ccc(N(c3ccc(-c4ccccc4)cc3)c3cc(-c4ccccc4)cc(-c4ccc5c(sc6c(-c7ccccc7)cccc65)c4-c4ccccc4)c3)cc21 ; c1ccc(-c2ccc(N(c3cc(-c4ccccc4)cc(-c4ccc5c(sc6c(-c7ccccc7)cccc65)c4-c4ccccc4)c3)c3cccc4c3-c3ccccc3C43c4ccccc4-c4ccccc43)cc2)cc1]

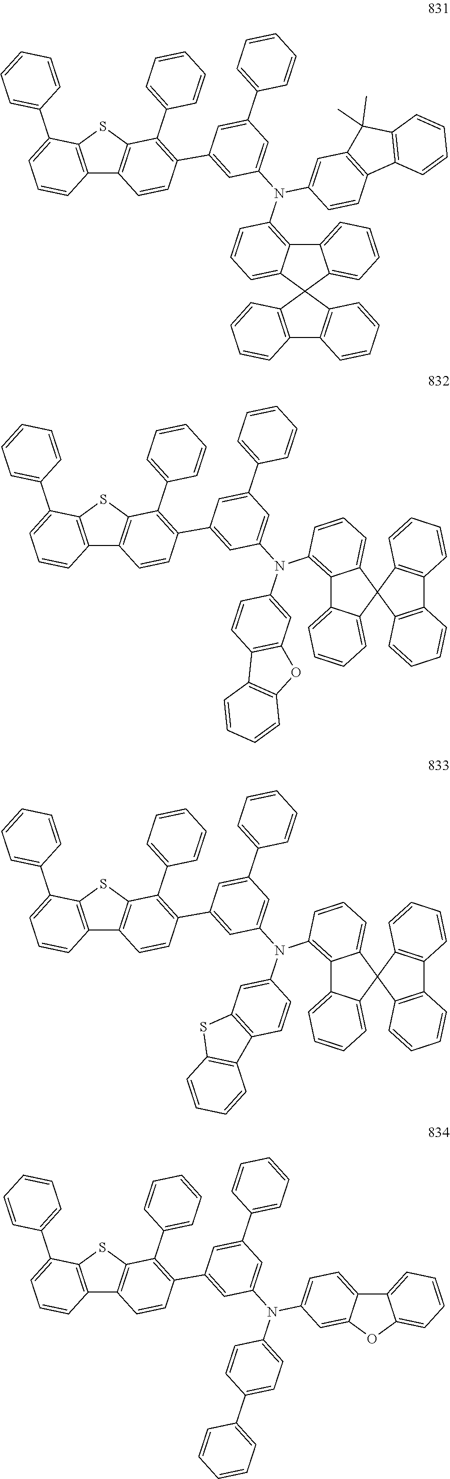

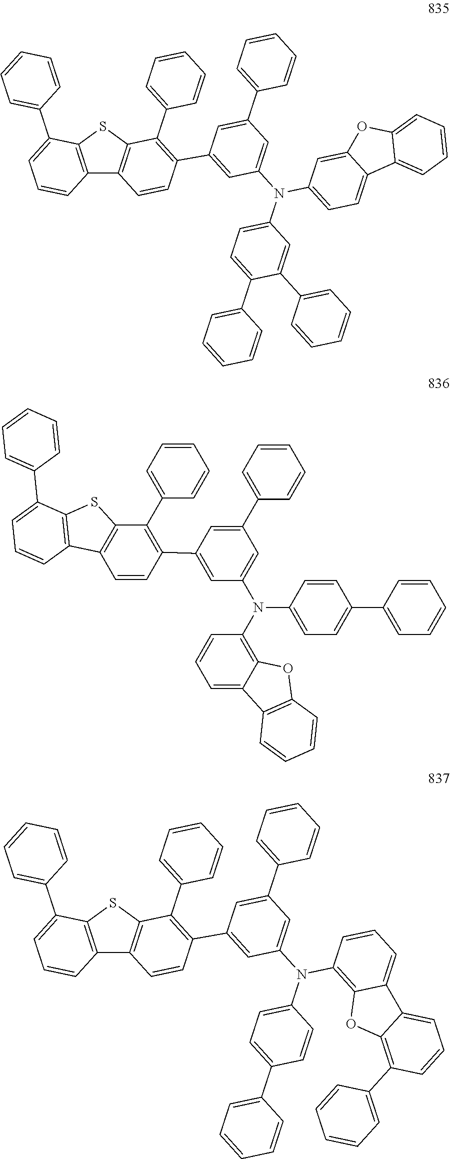 [SMILES: c1ccc(-c2cc(-c3ccc4c(sc5c(-c6ccccc6)cccc54)c3-c3ccccc3)cc(N(c3ccc(-c4ccccc4)c(-c4ccccc4)c3)c3ccc4c(c3)oc3ccccc34)c2)cc1 ; c1ccc(-c2ccc(N(c3cc(-c4ccccc4)cc(-c4ccc5c(sc6c(-c7ccccc7)cccc65)c4-c4ccccc4)c3)c3cccc4c3oc3ccccc34)cc2)cc1 ; c1ccc(-c2ccc(N(c3cc(-c4ccccc4)cc(-c4ccc5c(sc6c(-c7ccccc7)cccc65)c4-c4ccccc4)c3)c3cccc4c3oc3c(-c5ccccc5)cccc34)cc2)cc1]

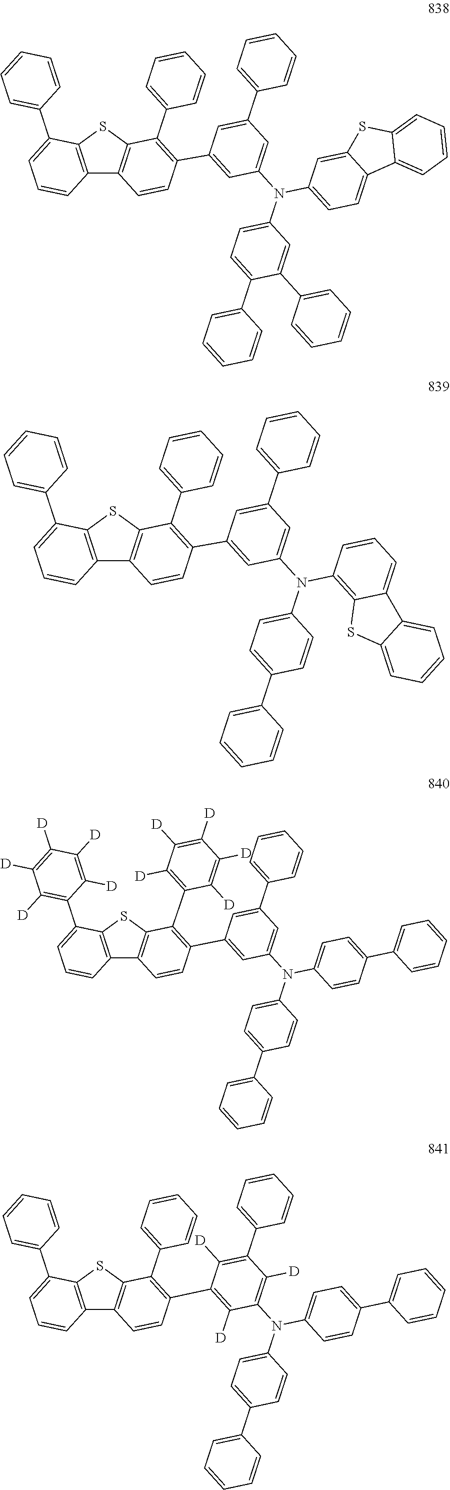

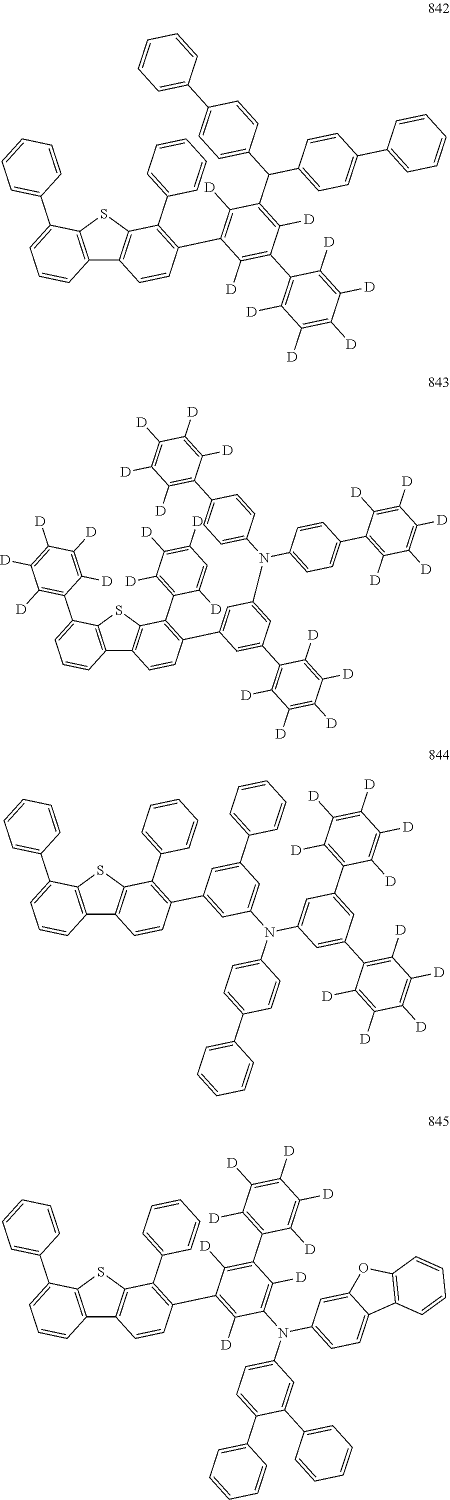

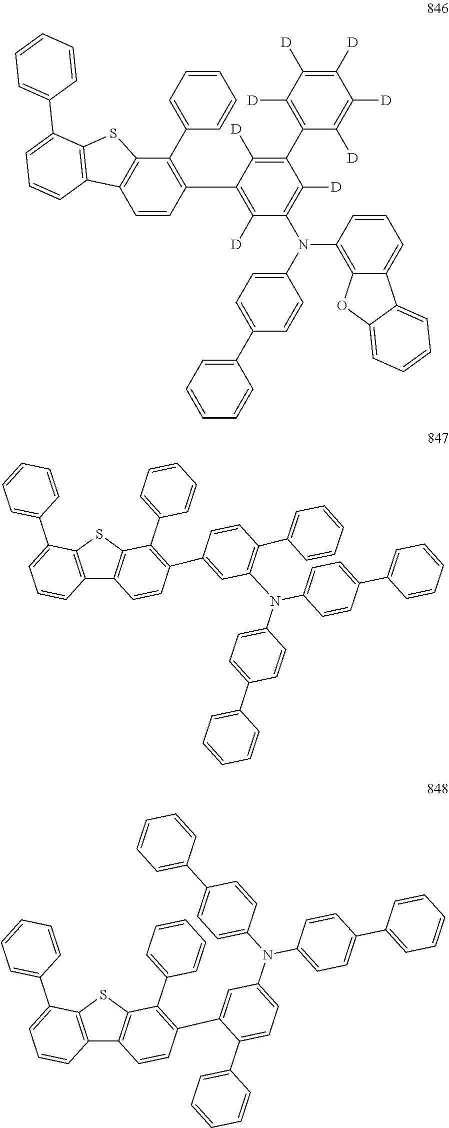 [SMILES: [2H]c1c([2H])c([2H])c(-c2c([2H])c(-c3ccc4c(sc5c(-c6ccccc6)cccc54)c3-c3ccccc3)c([2H])c(N(c3ccc(-c4ccccc4)cc3)c3cccc4c3oc3ccccc34)c2[2H])c([2H])c1[2H] ; c1ccc(-c2ccc(N(c3ccc(-c4ccccc4)cc3)c3cc(-c4ccc5c(sc6c(-c7ccccc7)cccc65)c4-c4ccccc4)ccc3-c3ccccc3)cc2)cc1 ; c1ccc(-c2ccc(N(c3ccc(-c4ccccc4)cc3)c3ccc(-c4ccccc4)c(-c4ccc5c(sc6c(-c7ccccc7)cccc65)c4-c4ccccc4)c3)cc2)cc1]

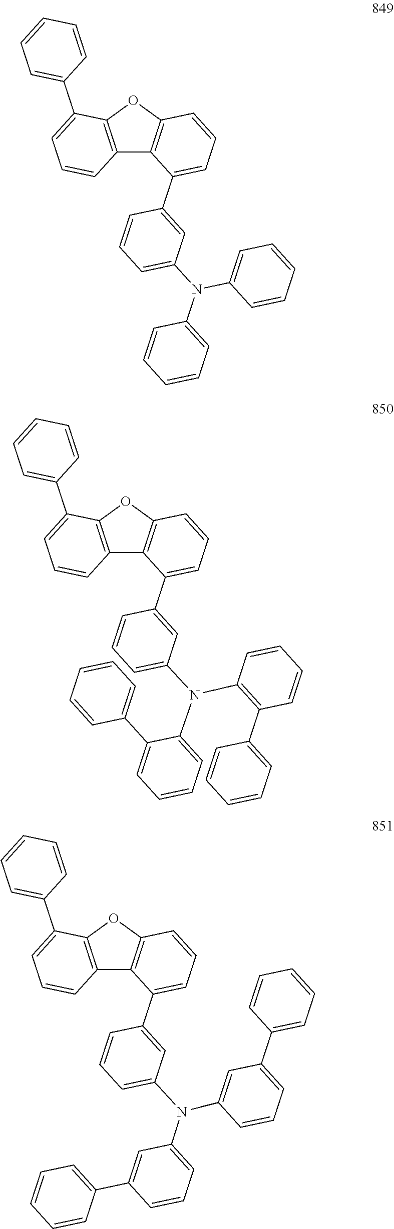 [SMILES: c1ccc(-c2cccc3c2oc2cccc(-c4cccc(N(c5ccccc5)c5ccccc5)c4)c23)cc1 ; c1ccc(-c2ccccc2N(c2cccc(-c3cccc4oc5c(-c6ccccc6)cccc5c34)c2)c2ccccc2-c2ccccc2)cc1 ; c1ccc(-c2cccc(N(c3cccc(-c4ccccc4)c3)c3cccc(-c4cccc5oc6c(-c7ccccc7)cccc6c45)c3)c2)cc1]

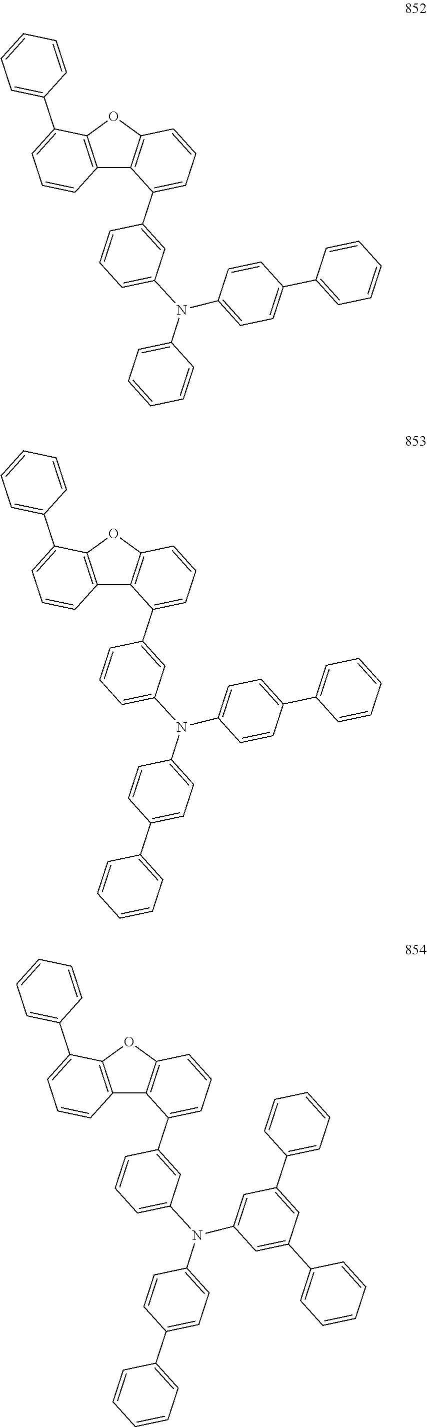 [SMILES: c1ccc(-c2ccc(N(c3ccccc3)c3cccc(-c4cccc5oc6c(-c7ccccc7)cccc6c45)c3)cc2)cc1 ; c1ccc(-c2ccc(N(c3ccc(-c4ccccc4)cc3)c3cccc(-c4cccc5oc6c(-c7ccccc7)cccc6c45)c3)cc2)cc1 ; c1ccc(-c2ccc(N(c3cc(-c4ccccc4)cc(-c4ccccc4)c3)c3cccc(-c4cccc5oc6c(-c7ccccc7)cccc6c45)c3)cc2)cc1]

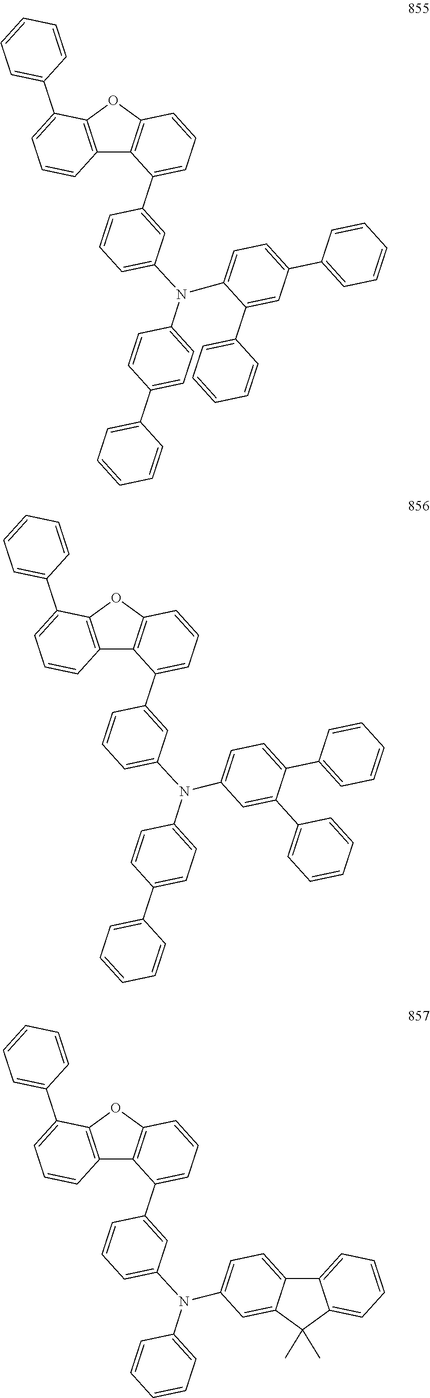 [SMILES: c1ccc(-c2ccc(N(c3cccc(-c4cccc5oc6c(-c7ccccc7)cccc6c45)c3)c3ccc(-c4ccccc4)cc3-c3ccccc3)cc2)cc1 ; c1ccc(-c2ccc(N(c3cccc(-c4cccc5oc6c(-c7ccccc7)cccc6c45)c3)c3ccc(-c4ccccc4)c(-c4ccccc4)c3)cc2)cc1 ; CC1(C)c2ccccc2-c2ccc(N(c3ccccc3)c3cccc(-c4cccc5oc6c(-c7ccccc7)cccc6c45)c3)cc21]

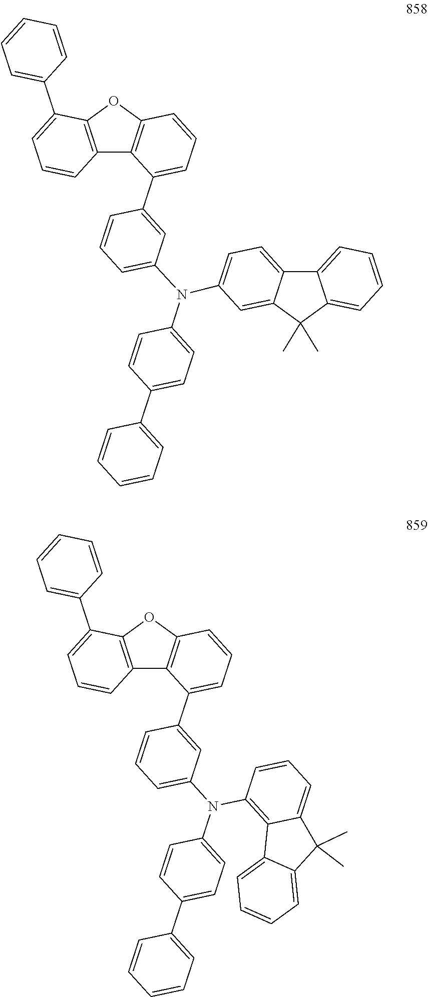 [SMILES: CC1(C)c2ccccc2-c2ccc(N(c3ccc(-c4ccccc4)cc3)c3cccc(-c4cccc5oc6c(-c7ccccc7)cccc6c45)c3)cc21 ; CC1(C)c2ccccc2-c2c(N(c3ccc(-c4ccccc4)cc3)c3cccc(-c4cccc5oc6c(-c7ccccc7)cccc6c45)c3)cccc21]

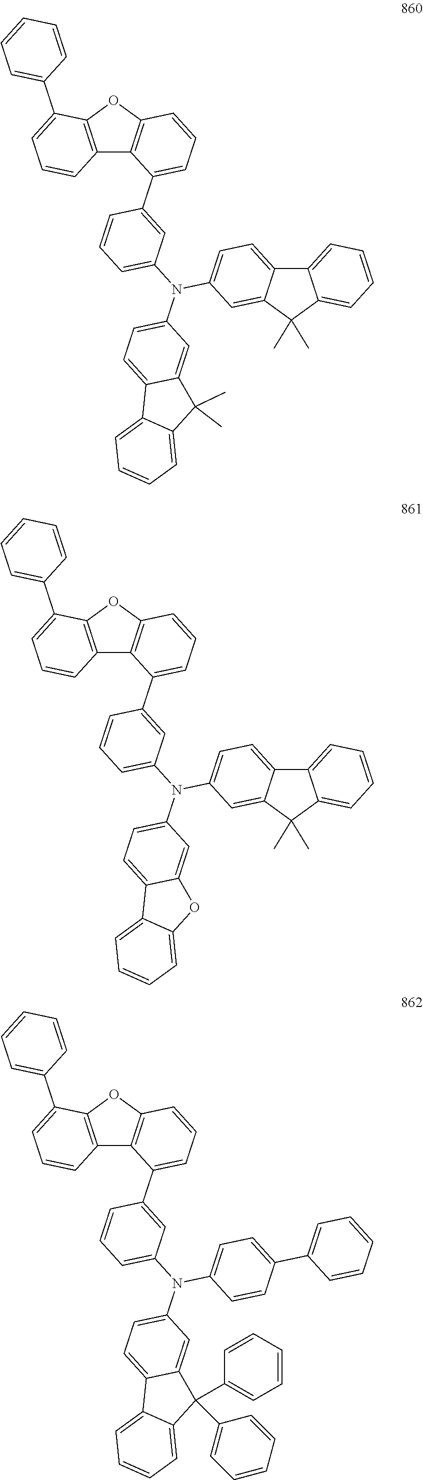 [SMILES: CC1(C)c2ccccc2-c2ccc(N(c3cccc(-c4cccc5oc6c(-c7ccccc7)cccc6c45)c3)c3ccc4c(c3)C(C)(C)c3ccccc3-4)cc21 ; CC1(C)c2ccccc2-c2ccc(N(c3cccc(-c4cccc5oc6c(-c7ccccc7)cccc6c45)c3)c3ccc4c(c3)oc3ccccc34)cc21 ; c1ccc(-c2ccc(N(c3cccc(-c4cccc5oc6c(-c7ccccc7)cccc6c45)c3)c3ccc4c(c3)C(c3ccccc3)(c3ccccc3)c3ccccc3-4)cc2)cc1]

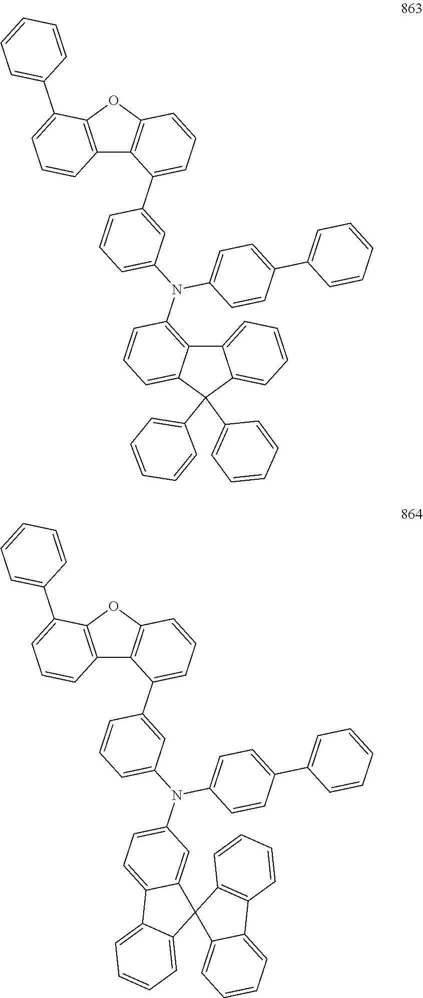 [SMILES: c1ccc(-c2ccc(N(c3cccc(-c4cccc5oc6c(-c7ccccc7)cccc6c45)c3)c3cccc4c3-c3ccccc3C4(c3ccccc3)c3ccccc3)cc2)cc1 ; c1ccc(-c2ccc(N(c3cccc(-c4cccc5oc6c(-c7ccccc7)cccc6c45)c3)c3ccc4c(c3)C3(c5ccccc5-c5ccccc53)c3ccccc3-4)cc2)cc1]

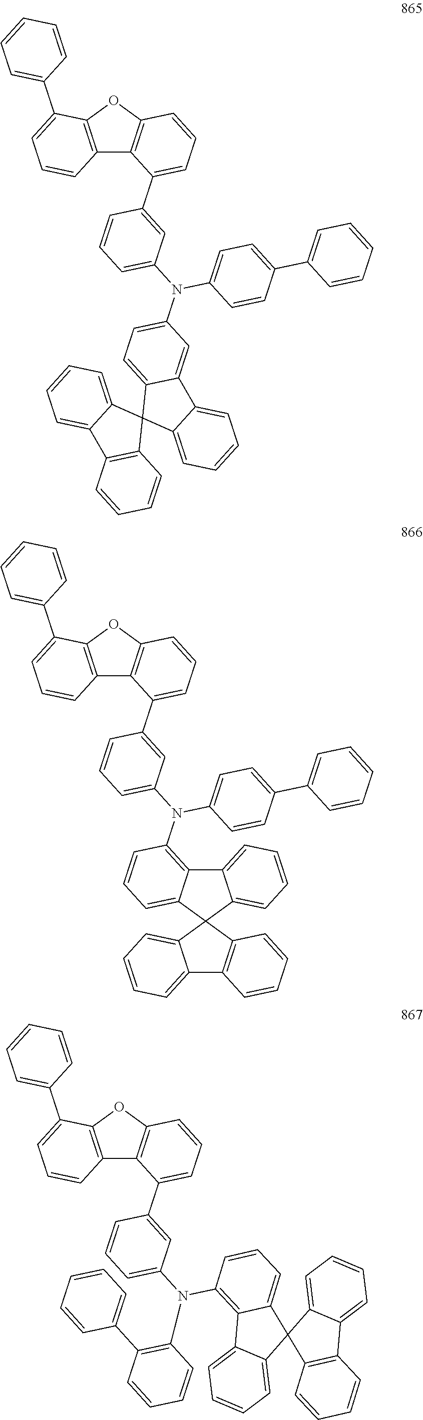 [SMILES: c1ccc(-c2ccc(N(c3cccc(-c4cccc5oc6c(-c7ccccc7)cccc6c45)c3)c3ccc4c(c3)-c3ccccc3C43c4ccccc4-c4ccccc43)cc2)cc1 ; c1ccc(-c2ccc(N(c3cccc(-c4cccc5oc6c(-c7ccccc7)cccc6c45)c3)c3cccc4c3-c3ccccc3C43c4ccccc4-c4ccccc43)cc2)cc1 ; c1ccc(-c2ccccc2N(c2cccc(-c3cccc4oc5c(-c6ccccc6)cccc5c34)c2)c2cccc3c2-c2ccccc2C32c3ccccc3-c3ccccc32)cc1]

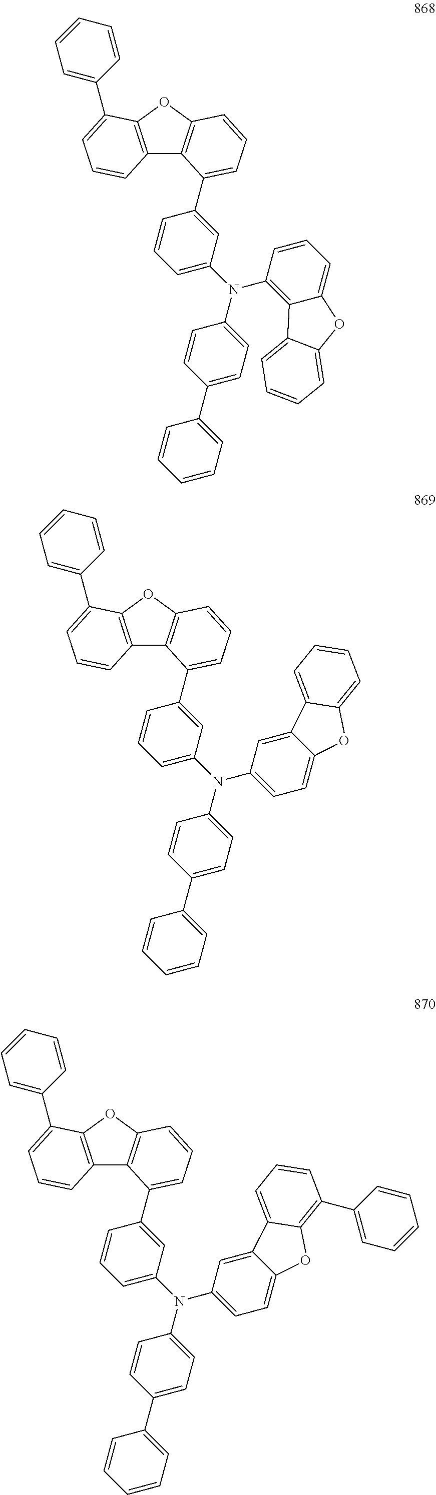 [SMILES: c1ccc(-c2ccc(N(c3cccc(-c4cccc5oc6c(-c7ccccc7)cccc6c45)c3)c3cccc4oc5ccccc5c34)cc2)cc1 ; c1ccc(-c2ccc(N(c3cccc(-c4cccc5oc6c(-c7ccccc7)cccc6c45)c3)c3ccc4oc5ccccc5c4c3)cc2)cc1 ; c1ccc(-c2ccc(N(c3cccc(-c4cccc5oc6c(-c7ccccc7)cccc6c45)c3)c3ccc4oc5c(-c6ccccc6)cccc5c4c3)cc2)cc1]

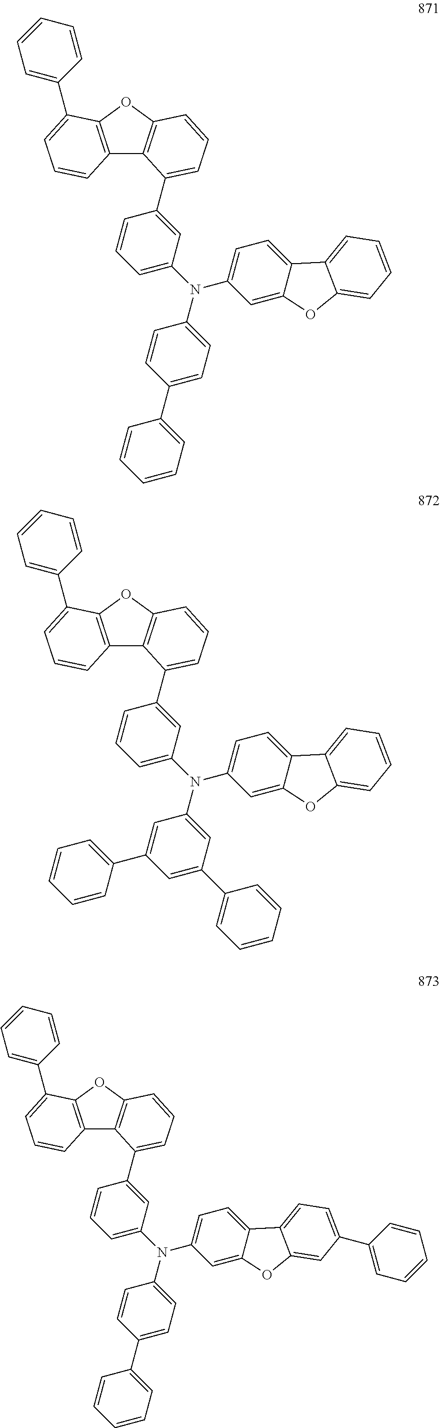 [SMILES: c1ccc(-c2ccc(N(c3cccc(-c4cccc5oc6c(-c7ccccc7)cccc6c45)c3)c3ccc4c(c3)oc3ccccc34)cc2)cc1 ; c1ccc(-c2cc(-c3ccccc3)cc(N(c3cccc(-c4cccc5oc6c(-c7ccccc7)cccc6c45)c3)c3ccc4c(c3)oc3ccccc34)c2)cc1 ; c1ccc(-c2ccc(N(c3cccc(-c4cccc5oc6c(-c7ccccc7)cccc6c45)c3)c3ccc4c(c3)oc3cc(-c5ccccc5)ccc34)cc2)cc1]

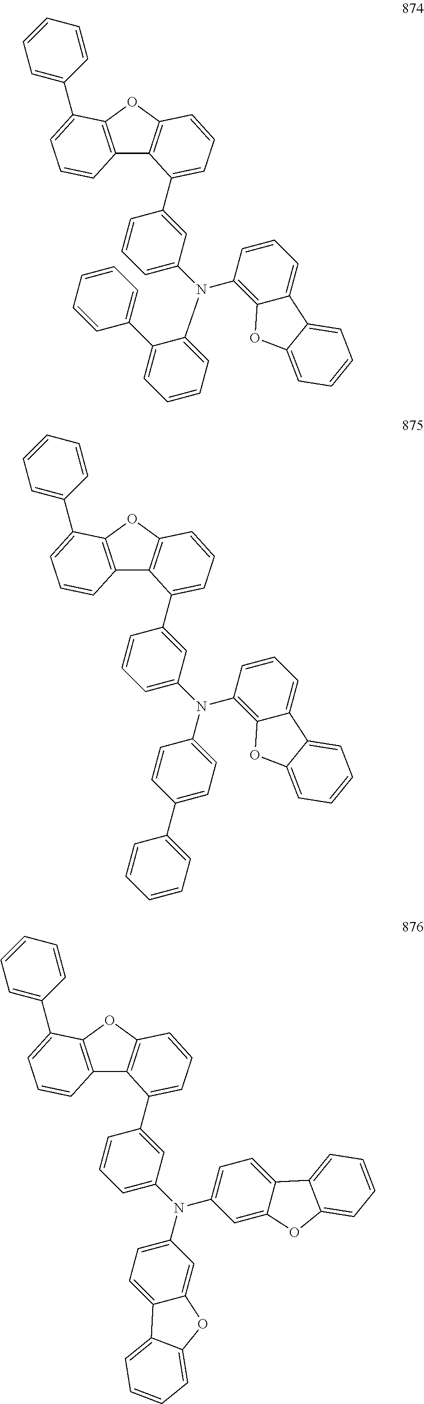 [SMILES: c1ccc(-c2ccccc2N(c2cccc(-c3cccc4oc5c(-c6ccccc6)cccc5c34)c2)c2cccc3c2oc2ccccc23)cc1 ; c1ccc(-c2ccc(N(c3cccc(-c4cccc5oc6c(-c7ccccc7)cccc6c45)c3)c3cccc4c3oc3ccccc34)cc2)cc1 ; c1ccc(-c2cccc3c2oc2cccc(-c4cccc(N(c5ccc6c(c5)oc5ccccc56)c5ccc6c(c5)oc5ccccc56)c4)c23)cc1]

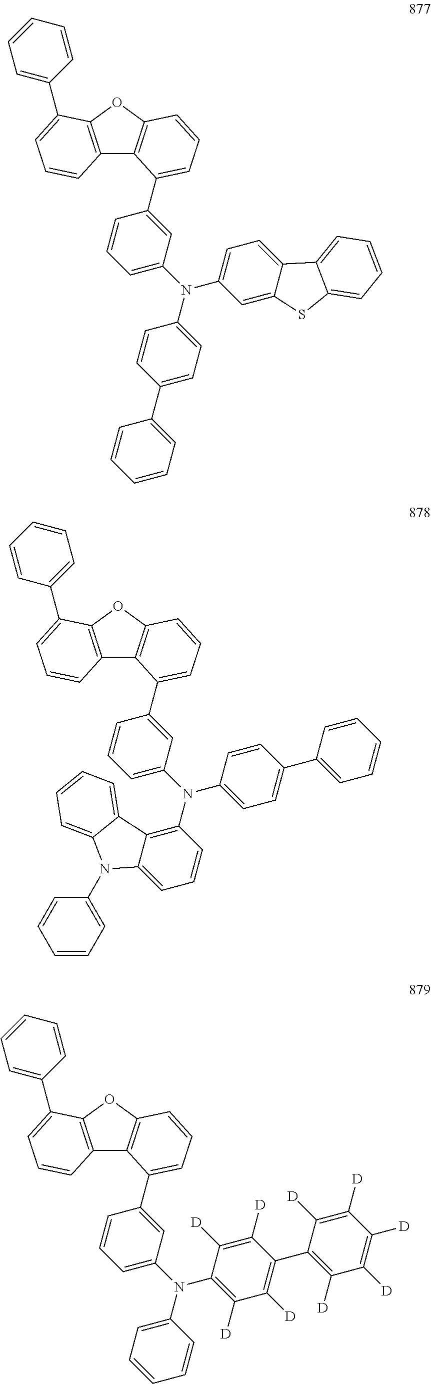 [SMILES: c1ccc(-c2ccc(N(c3cccc(-c4cccc5oc6c(-c7ccccc7)cccc6c45)c3)c3ccc4c(c3)sc3ccccc34)cc2)cc1 ; c1ccc(-c2ccc(N(c3cccc(-c4cccc5oc6c(-c7ccccc7)cccc6c45)c3)c3cccc4c3c3ccccc3n4-c3ccccc3)cc2)cc1 ; [2H]c1c([2H])c([2H])c(-c2c([2H])c([2H])c(N(c3ccccc3)c3cccc(-c4cccc5oc6c(-c7ccccc7)cccc6c45)c3)c([2H])c2[2H])c([2H])c1[2H]]

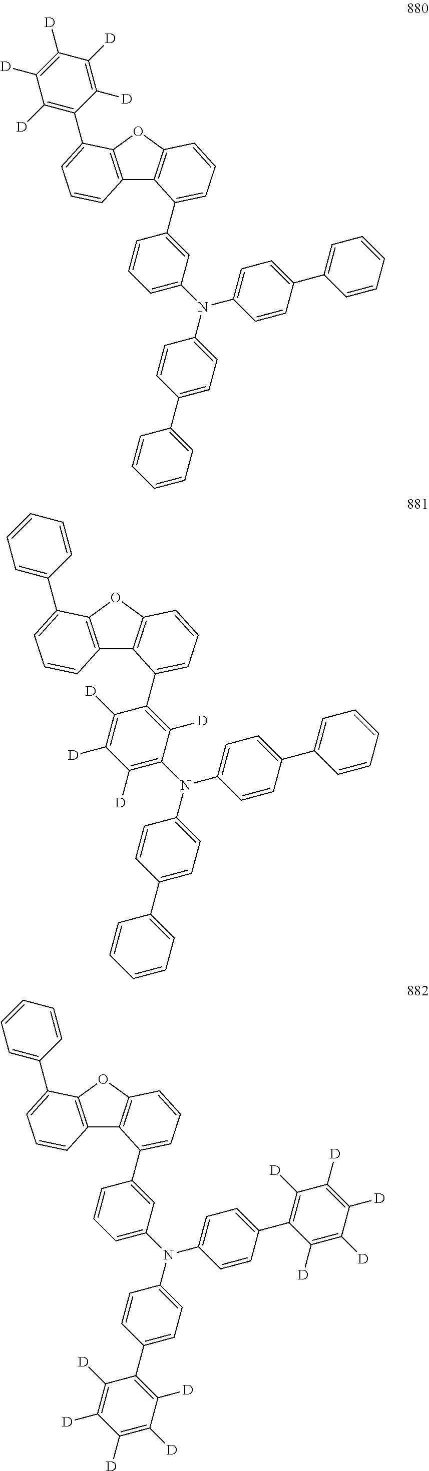 [SMILES: [2H]c1c([2H])c([2H])c(-c2cccc3c2oc2cccc(-c4cccc(N(c5ccc(-c6ccccc6)cc5)c5ccc(-c6ccccc6)cc5)c4)c23)c([2H])c1[2H] ; [2H]c1c([2H])c(-c2cccc3oc4c(-c5ccccc5)cccc4c23)c([2H])c(N(c2ccc(-c3ccccc3)cc2)c2ccc(-c3ccccc3)cc2)c1[2H] ; [2H]c1c([2H])c([2H])c(-c2ccc(N(c3ccc(-c4c([2H])c([2H])c([2H])c([2H])c4[2H])cc3)c3cccc(-c4cccc5oc6c(-c7ccccc7)cccc6c45)c3)cc2)c([2H])c1[2H]]

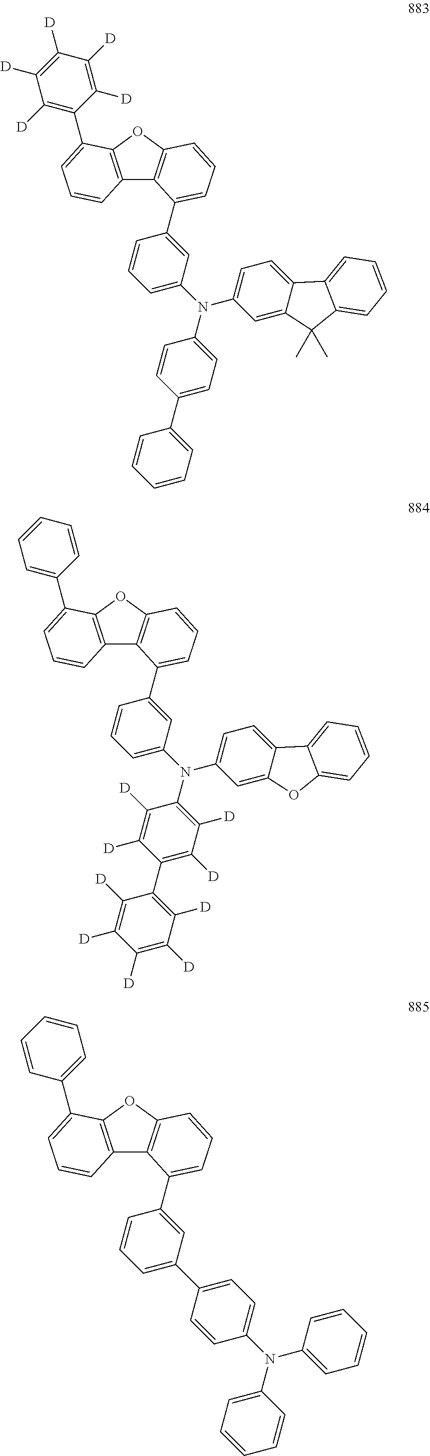 [SMILES: [2H]c1c([2H])c([2H])c(-c2cccc3c2oc2cccc(-c4cccc(N(c5ccc(-c6ccccc6)cc5)c5ccc6c(c5)C(C)(C)c5ccccc5-6)c4)c23)c([2H])c1[2H] ; [2H]c1c([2H])c([2H])c(-c2c([2H])c([2H])c(N(c3cccc(-c4cccc5oc6c(-c7ccccc7)cccc6c45)c3)c3ccc4c(c3)oc3ccccc34)c([2H])c2[2H])c([2H])c1[2H] ; c1ccc(-c2cccc3c2oc2cccc(-c4cccc(-c5ccc(N(c6ccccc6)c6ccccc6)cc5)c4)c23)cc1]

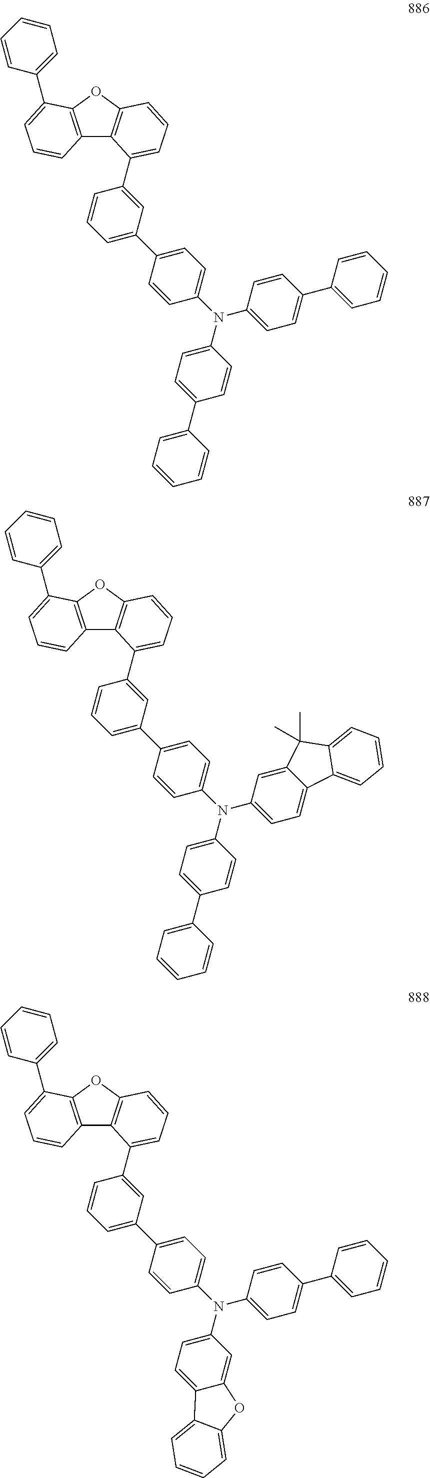 [SMILES: c1ccc(-c2ccc(N(c3ccc(-c4ccccc4)cc3)c3ccc(-c4cccc(-c5cccc6oc7c(-c8ccccc8)cccc7c56)c4)cc3)cc2)cc1 ; CC1(C)c2ccccc2-c2ccc(N(c3ccc(-c4ccccc4)cc3)c3ccc(-c4cccc(-c5cccc6oc7c(-c8ccccc8)cccc7c56)c4)cc3)cc21 ; c1ccc(-c2ccc(N(c3ccc(-c4cccc(-c5cccc6oc7c(-c8ccccc8)cccc7c56)c4)cc3)c3ccc4c(c3)oc3ccccc34)cc2)cc1]

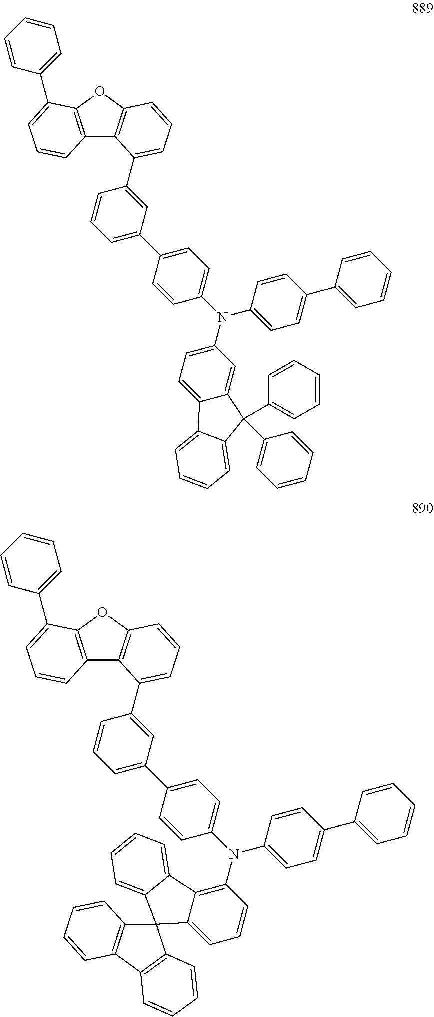 [SMILES: c1ccc(-c2ccc(N(c3ccc(-c4cccc(-c5cccc6oc7c(-c8ccccc8)cccc7c56)c4)cc3)c3ccc4c(c3)C(c3ccccc3)(c3ccccc3)c3ccccc3-4)cc2)cc1 ; c1ccc(-c2ccc(N(c3ccc(-c4cccc(-c5cccc6oc7c(-c8ccccc8)cccc7c56)c4)cc3)c3cccc4c3-c3ccccc3C43c4ccccc4-c4ccccc43)cc2)cc1]

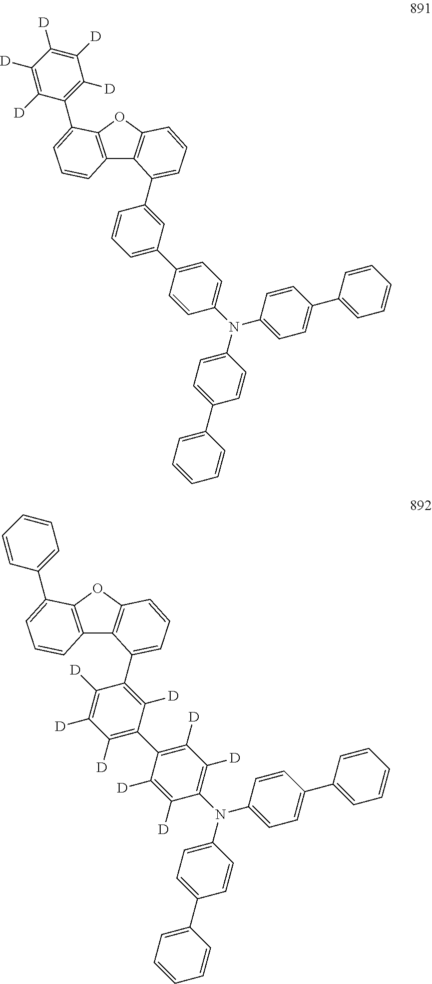 [SMILES: [2H]c1c([2H])c([2H])c(-c2cccc3c2oc2cccc(-c4cccc(-c5ccc(N(c6ccc(-c7ccccc7)cc6)c6ccc(-c7ccccc7)cc6)cc5)c4)c23)c([2H])c1[2H] ; [2H]c1c([2H])c(-c2c([2H])c([2H])c(N(c3ccc(-c4ccccc4)cc3)c3ccc(-c4ccccc4)cc3)c([2H])c2[2H])c([2H])c(-c2cccc3oc4c(-c5ccccc5)cccc4c23)c1[2H]]

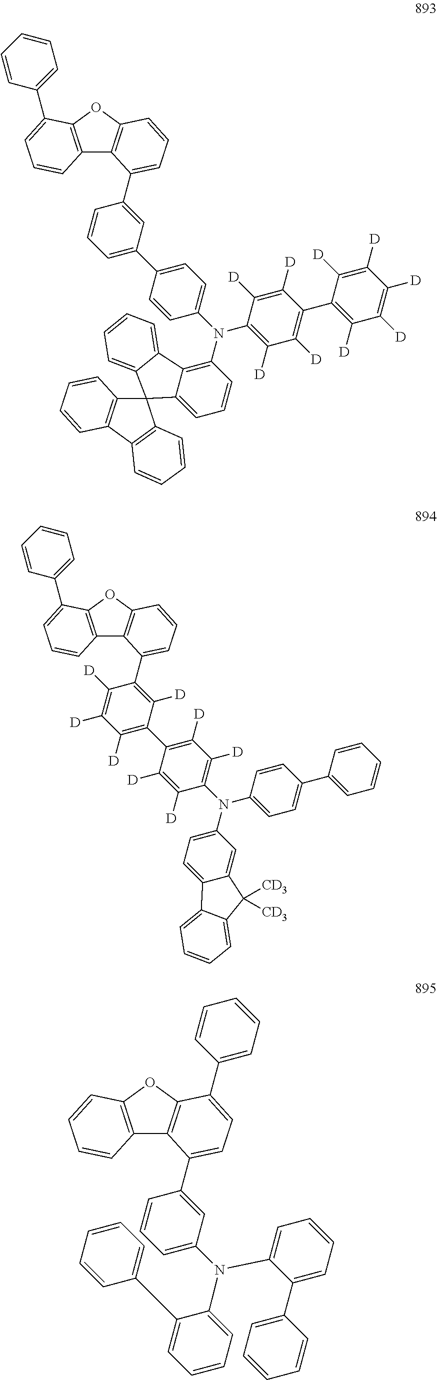 [SMILES: [2H]c1c([2H])c([2H])c(-c2c([2H])c([2H])c(N(c3ccc(-c4cccc(-c5cccc6oc7c(-c8ccccc8)cccc7c56)c4)cc3)c3cccc4c3-c3ccccc3C43c4ccccc4-c4ccccc43)c([2H])c2[2H])c([2H])c1[2H] ; [2H]c1c([2H])c(-c2c([2H])c([2H])c(N(c3ccc(-c4ccccc4)cc3)c3ccc4c(c3)C(C([2H])([2H])[2H])(C([2H])([2H])[2H])c3ccccc3-4)c([2H])c2[2H])c([2H])c(-c2cccc3oc4c(-c5ccccc5)cccc4c23)c1[2H] ; c1ccc(-c2ccccc2N(c2cccc(-c3ccc(-c4ccccc4)c4oc5ccccc5c34)c2)c2ccccc2-c2ccccc2)cc1]

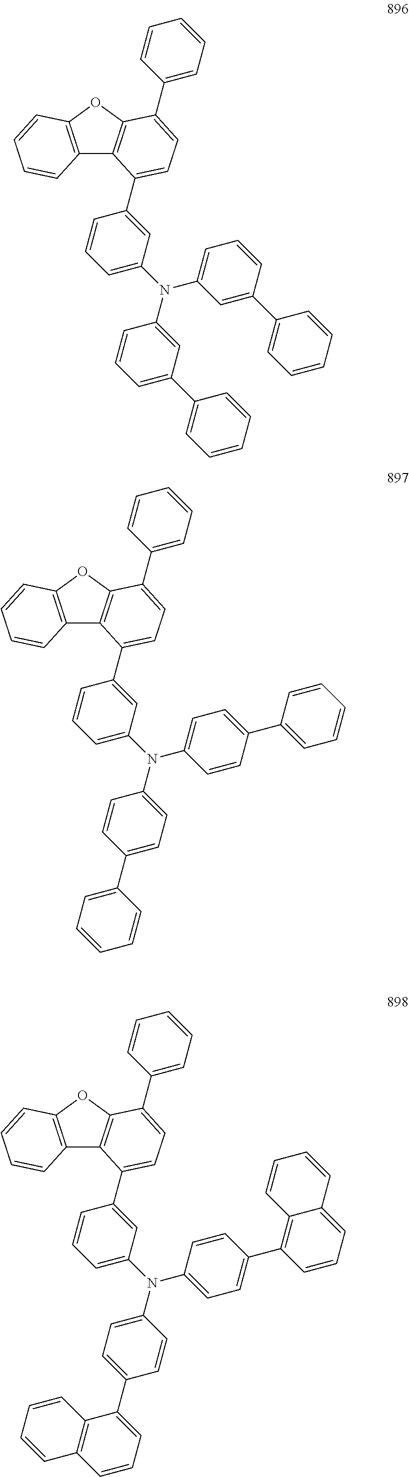 [SMILES: c1ccc(-c2cccc(N(c3cccc(-c4ccccc4)c3)c3cccc(-c4ccc(-c5ccccc5)c5oc6ccccc6c45)c3)c2)cc1 ; c1ccc(-c2ccc(N(c3ccc(-c4ccccc4)cc3)c3cccc(-c4ccc(-c5ccccc5)c5oc6ccccc6c45)c3)cc2)cc1 ; c1ccc(-c2ccc(-c3cccc(N(c4ccc(-c5cccc6ccccc56)cc4)c4ccc(-c5cccc6ccccc56)cc4)c3)c3c2oc2ccccc23)cc1]

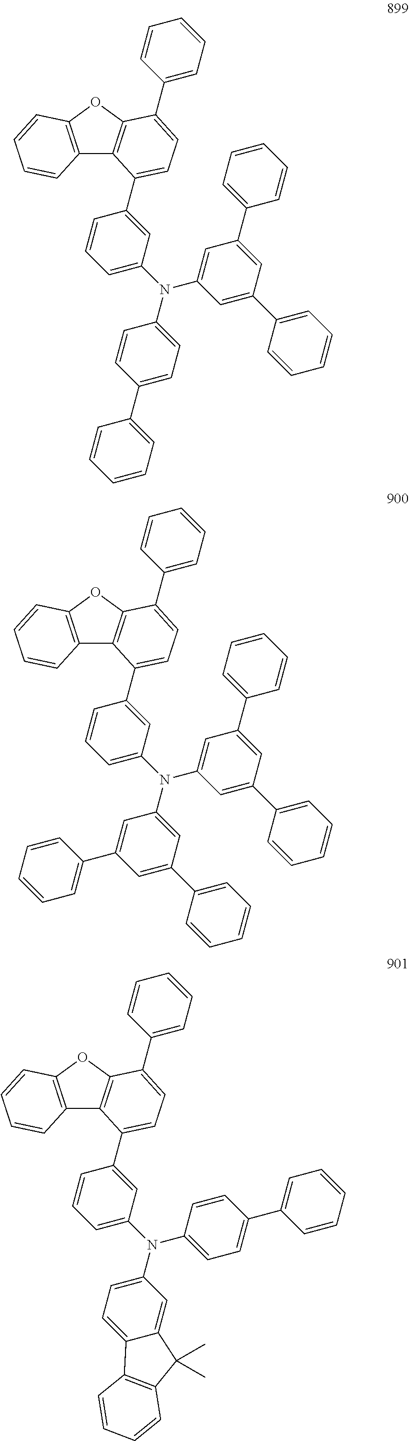

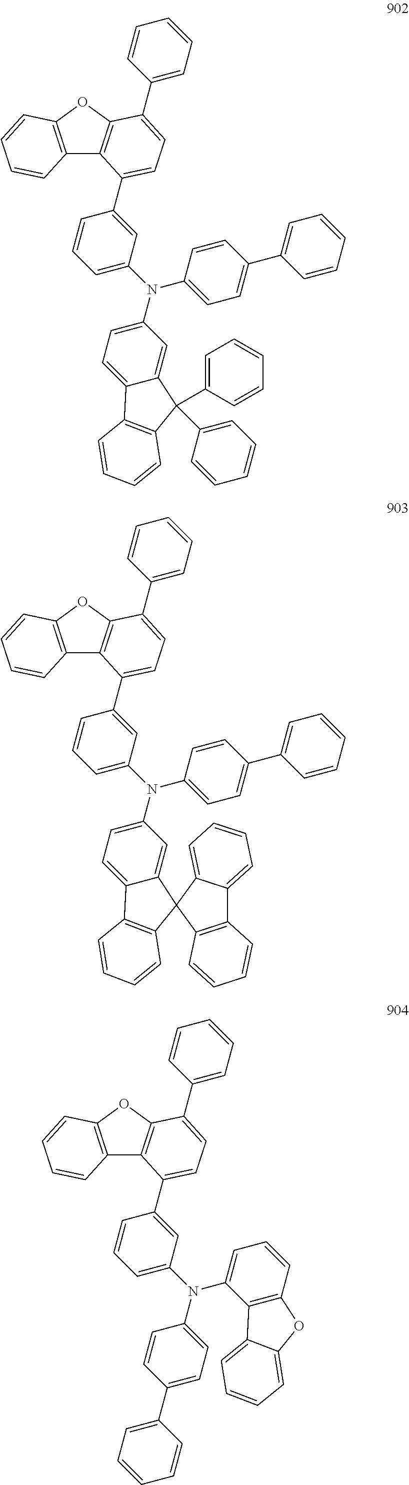

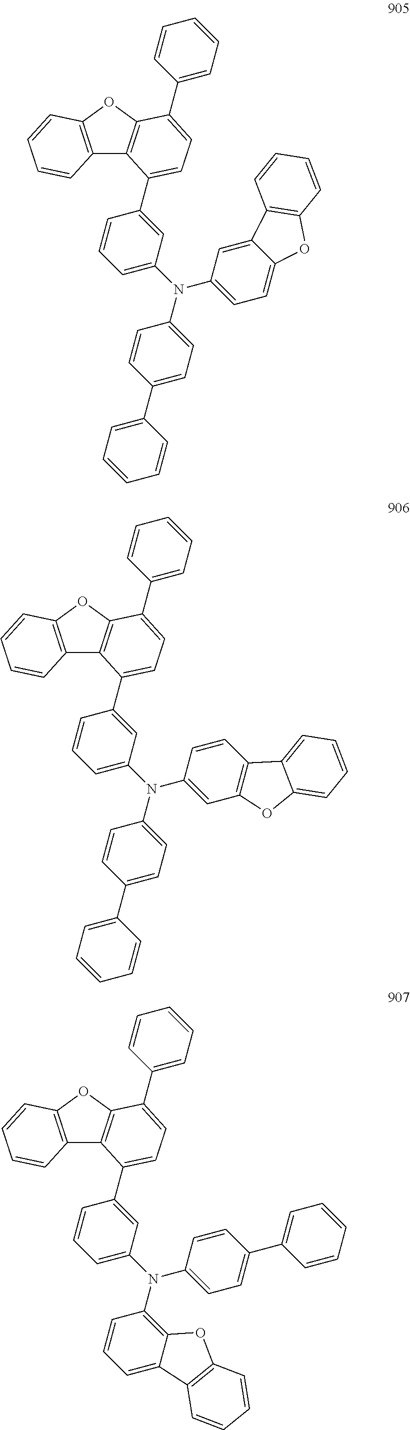

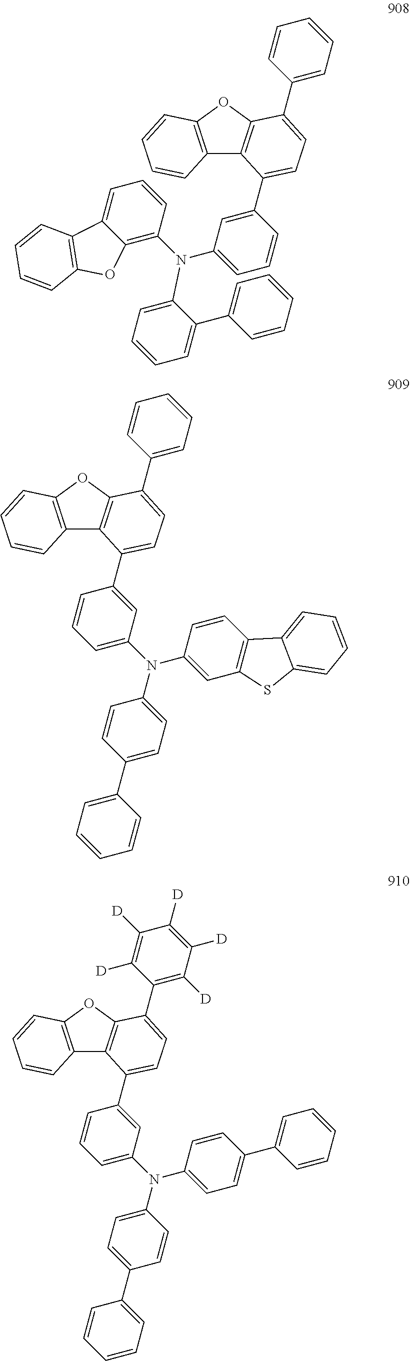

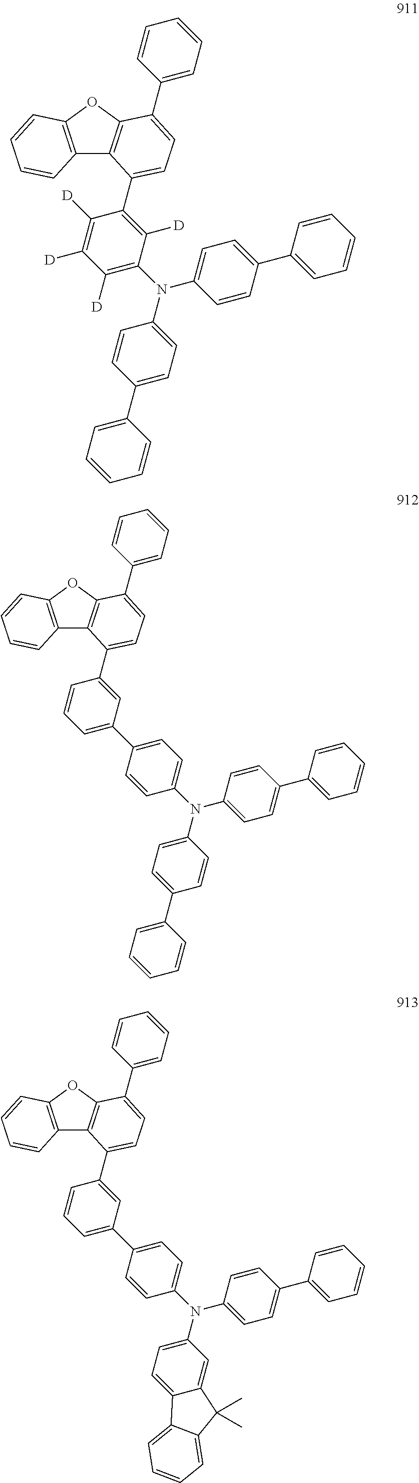

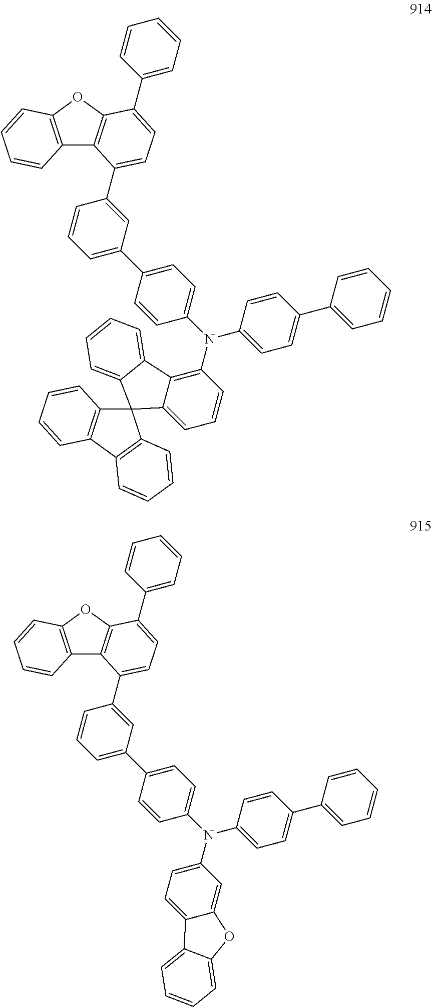

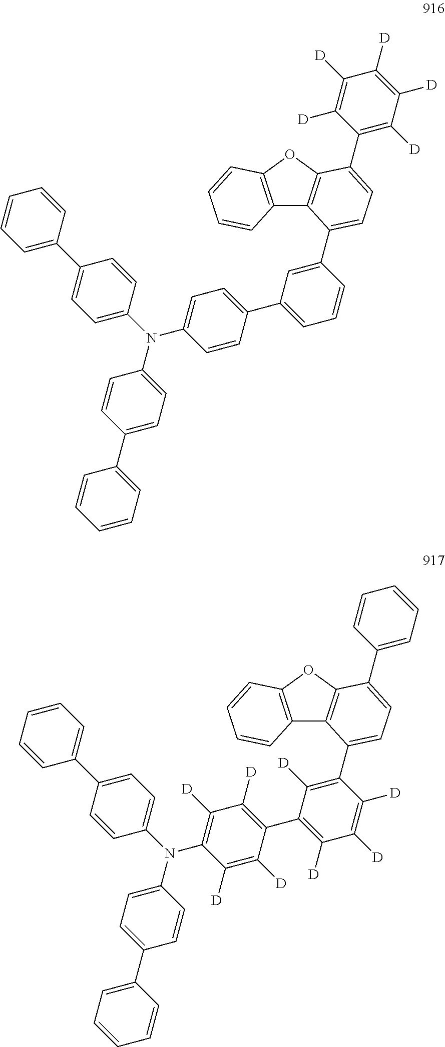 [SMILES: [2H]c1c([2H])c([2H])c(-c2ccc(-c3cccc(-c4ccc(N(c5ccc(-c6ccccc6)cc5)c5ccc(-c6ccccc6)cc5)cc4)c3)c3c2oc2ccccc23)c([2H])c1[2H] ; [2H]c1c([2H])c(-c2c([2H])c([2H])c(N(c3ccc(-c4ccccc4)cc3)c3ccc(-c4ccccc4)cc3)c([2H])c2[2H])c([2H])c(-c2ccc(-c3ccccc3)c3oc4ccccc4c23)c1[2H]]

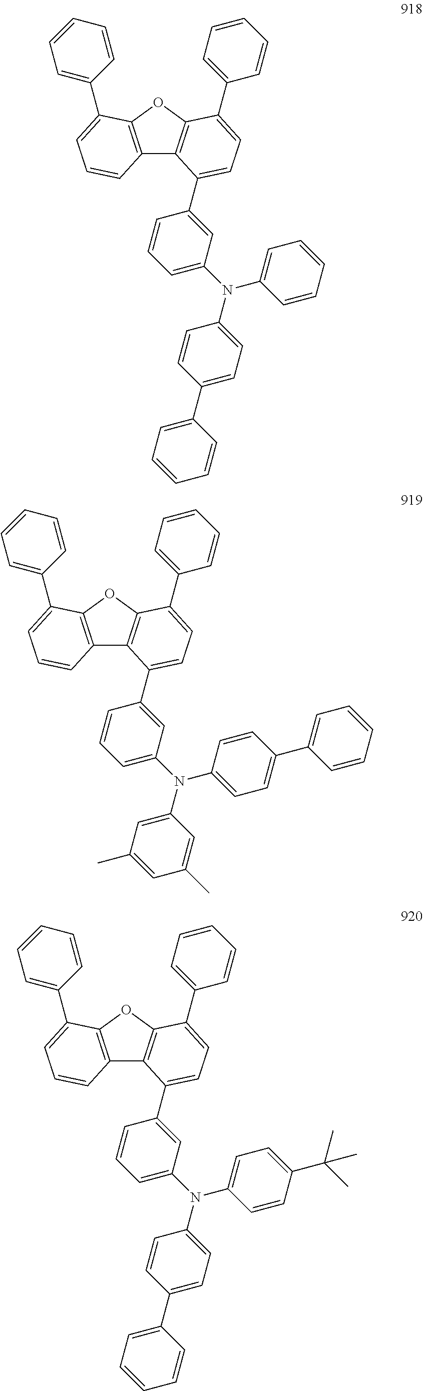

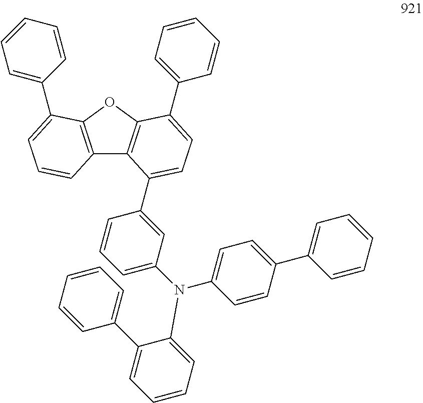 [SMILES: c1ccc(-c2ccc(N(c3cccc(-c4ccc(-c5ccccc5)c5oc6c(-c7ccccc7)cccc6c45)c3)c3ccccc3-c3ccccc3)cc2)cc1]

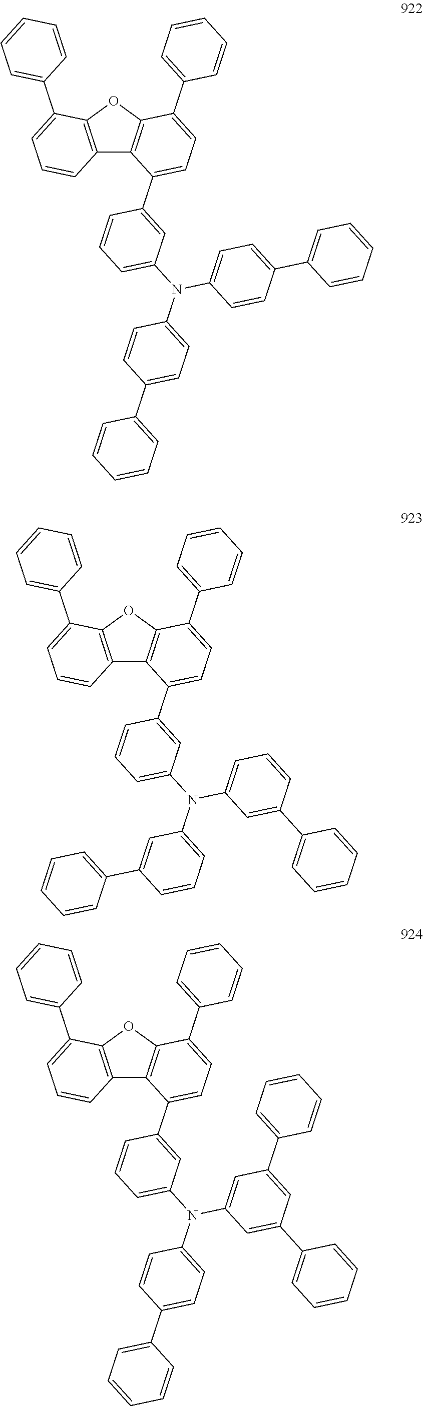

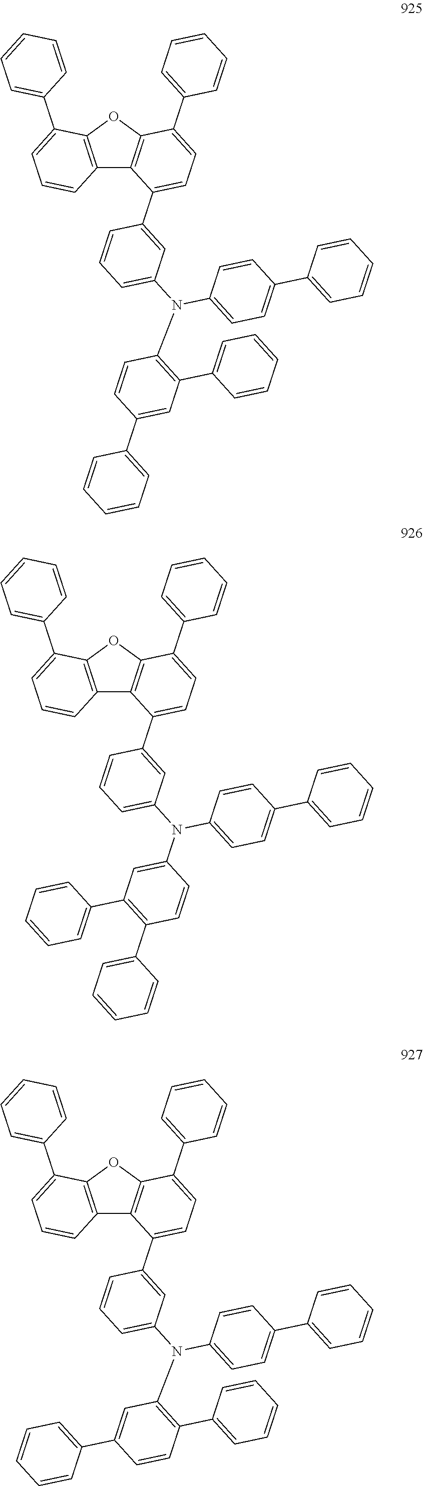 [SMILES: c1ccc(-c2ccc(N(c3cccc(-c4ccc(-c5ccccc5)c5oc6c(-c7ccccc7)cccc6c45)c3)c3ccc(-c4ccccc4)cc3-c3ccccc3)cc2)cc1 ; c1ccc(-c2ccc(N(c3cccc(-c4ccc(-c5ccccc5)c5oc6c(-c7ccccc7)cccc6c45)c3)c3ccc(-c4ccccc4)c(-c4ccccc4)c3)cc2)cc1 ; c1ccc(-c2ccc(N(c3cccc(-c4ccc(-c5ccccc5)c5oc6c(-c7ccccc7)cccc6c45)c3)c3cc(-c4ccccc4)ccc3-c3ccccc3)cc2)cc1]

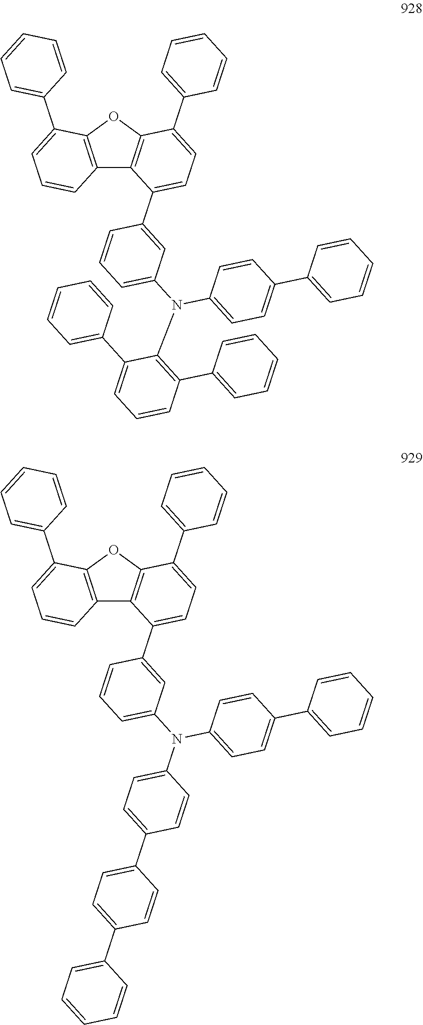 [SMILES: c1ccc(-c2ccc(N(c3cccc(-c4ccc(-c5ccccc5)c5oc6c(-c7ccccc7)cccc6c45)c3)c3c(-c4ccccc4)cccc3-c3ccccc3)cc2)cc1 ; c1ccc(-c2ccc(-c3ccc(N(c4ccc(-c5ccccc5)cc4)c4cccc(-c5ccc(-c6ccccc6)c6oc7c(-c8ccccc8)cccc7c56)c4)cc3)cc2)cc1]

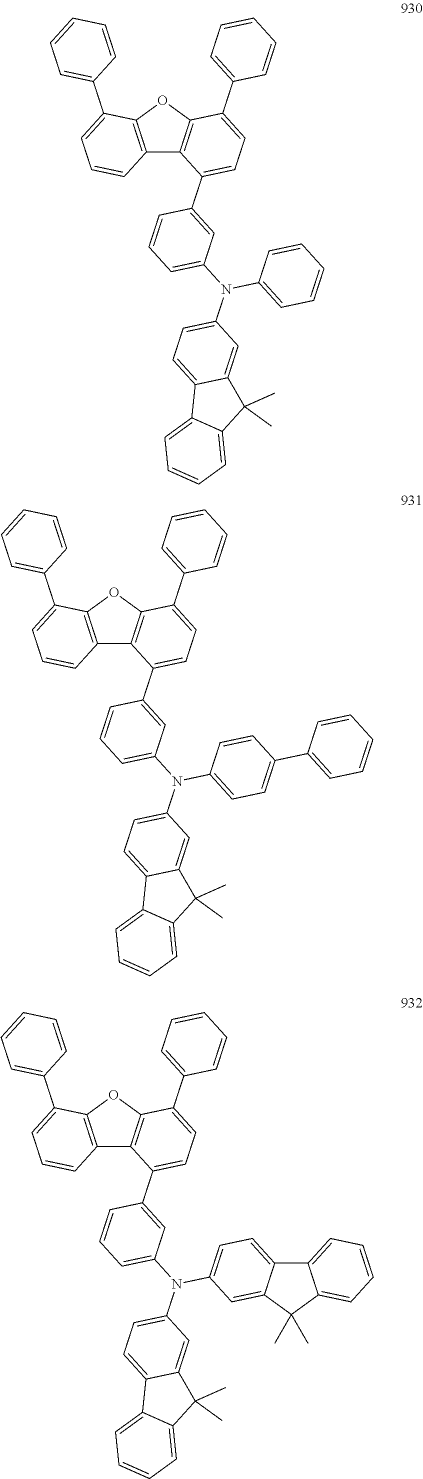

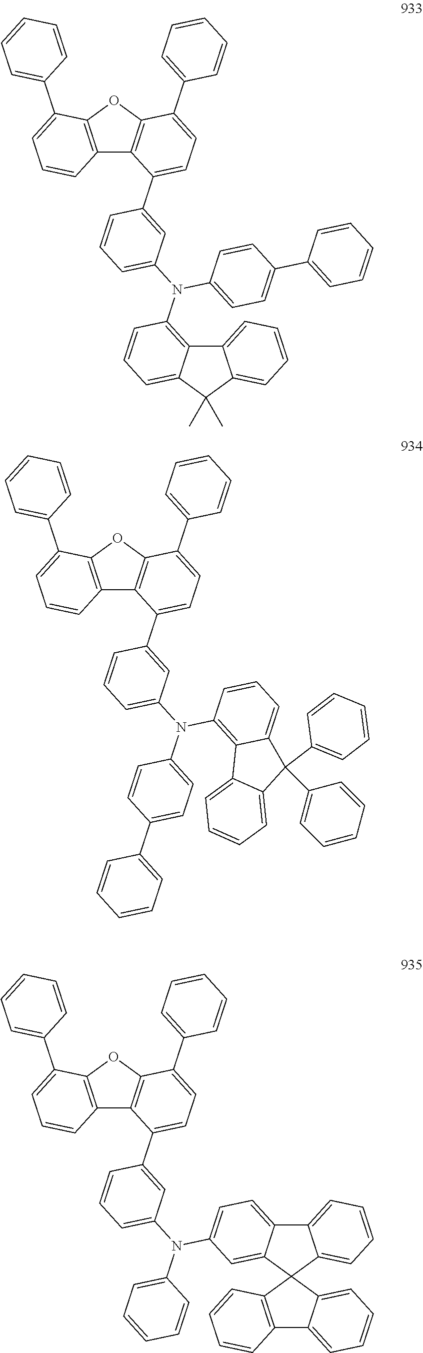

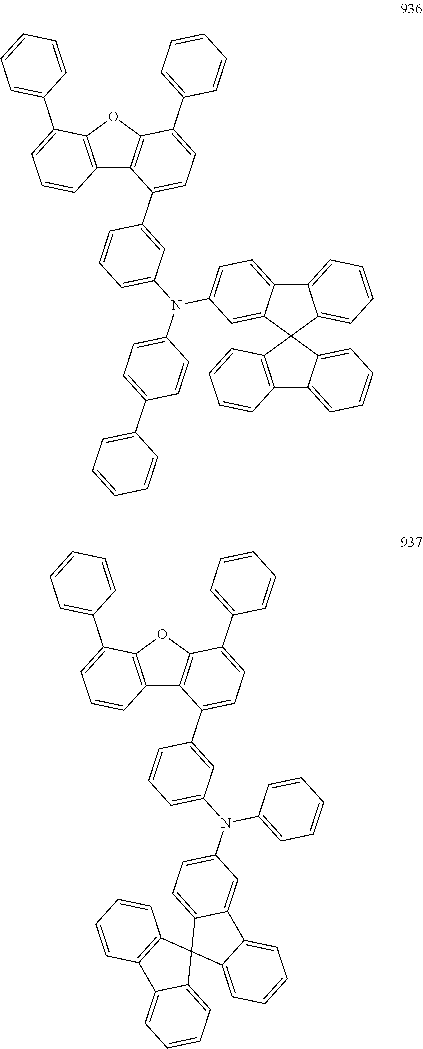 [SMILES: c1ccc(-c2ccc(N(c3cccc(-c4ccc(-c5ccccc5)c5oc6c(-c7ccccc7)cccc6c45)c3)c3ccc4c(c3)C3(c5ccccc5-c5ccccc53)c3ccccc3-4)cc2)cc1 ; c1ccc(-c2cccc3c2oc2c(-c4ccccc4)ccc(-c4cccc(N(c5ccccc5)c5ccc6c(c5)-c5ccccc5C65c6ccccc6-c6ccccc65)c4)c23)cc1]

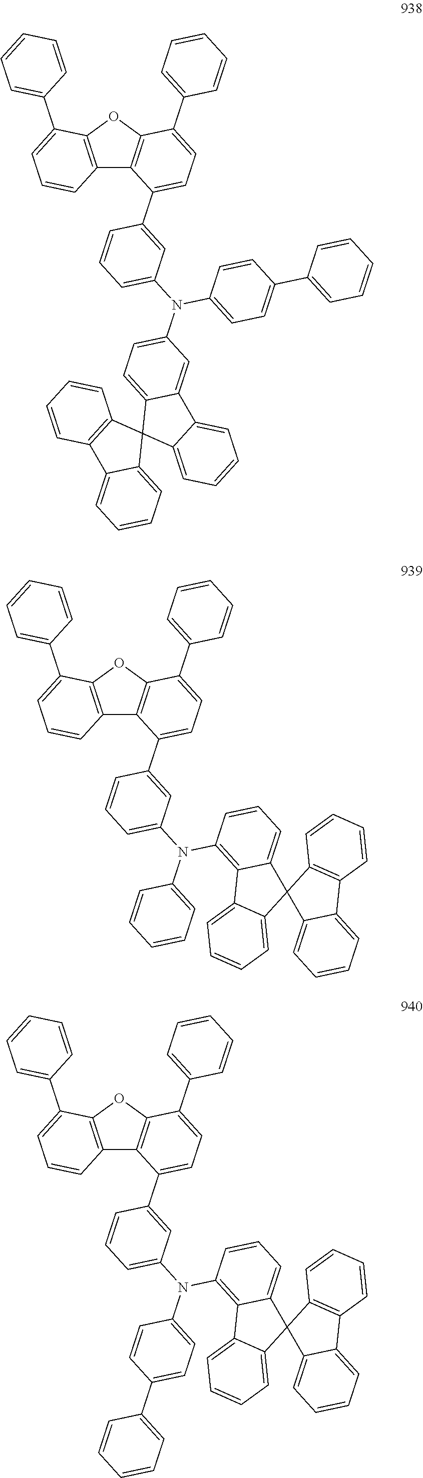 [SMILES: c1ccc(-c2ccc(N(c3cccc(-c4ccc(-c5ccccc5)c5oc6c(-c7ccccc7)cccc6c45)c3)c3ccc4c(c3)-c3ccccc3C43c4ccccc4-c4ccccc43)cc2)cc1 ; c1ccc(-c2cccc3c2oc2c(-c4ccccc4)ccc(-c4cccc(N(c5ccccc5)c5cccc6c5-c5ccccc5C65c6ccccc6-c6ccccc65)c4)c23)cc1 ; c1ccc(-c2ccc(N(c3cccc(-c4ccc(-c5ccccc5)c5oc6c(-c7ccccc7)cccc6c45)c3)c3cccc4c3-c3ccccc3C43c4ccccc4-c4ccccc43)cc2)cc1]

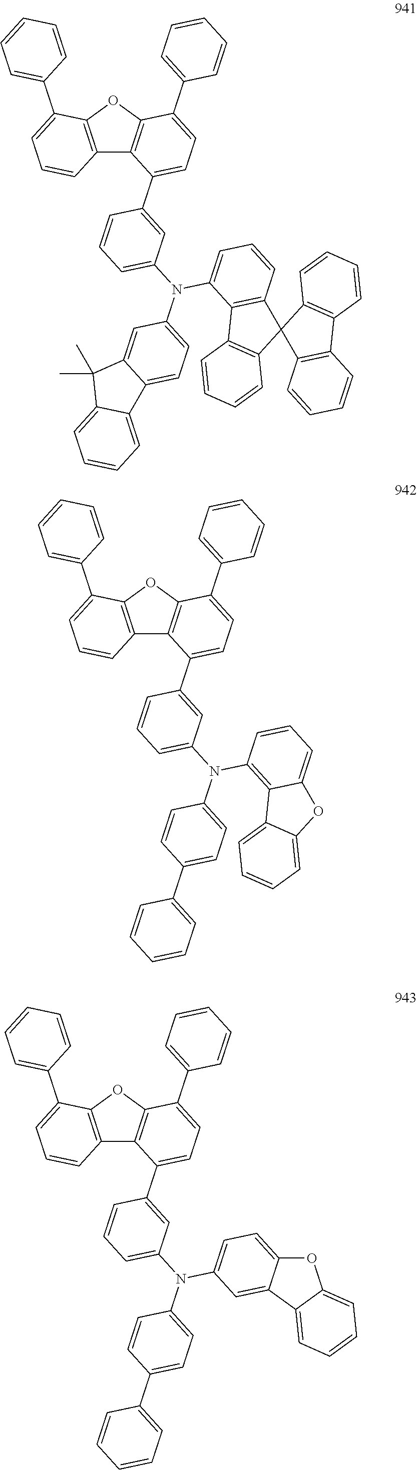

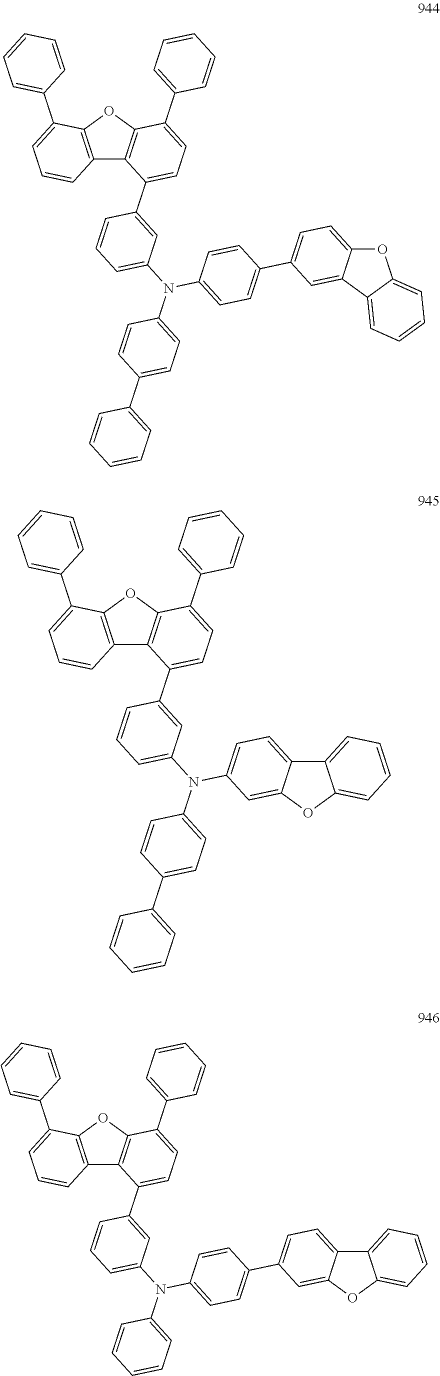 [SMILES: c1ccc(-c2ccc(N(c3ccc(-c4ccc5oc6ccccc6c5c4)cc3)c3cccc(-c4ccc(-c5ccccc5)c5oc6c(-c7ccccc7)cccc6c45)c3)cc2)cc1 ; c1ccc(-c2ccc(N(c3cccc(-c4ccc(-c5ccccc5)c5oc6c(-c7ccccc7)cccc6c45)c3)c3ccc4c(c3)oc3ccccc34)cc2)cc1 ; c1ccc(-c2cccc3c2oc2c(-c4ccccc4)ccc(-c4cccc(N(c5ccccc5)c5ccc(-c6ccc7c(c6)oc6ccccc67)cc5)c4)c23)cc1]

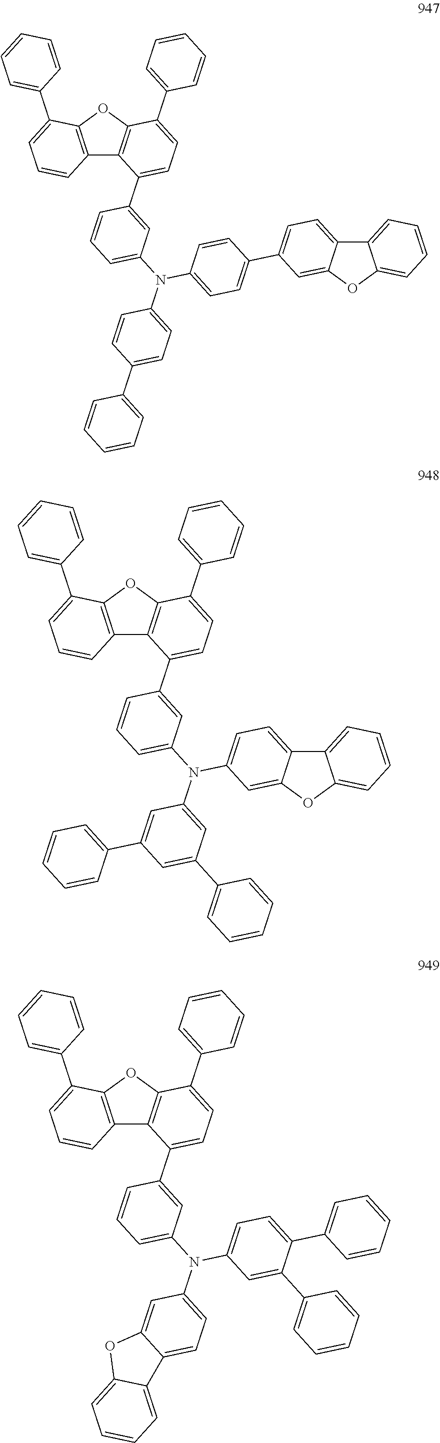 [SMILES: c1ccc(-c2ccc(N(c3ccc(-c4ccc5c(c4)oc4ccccc45)cc3)c3cccc(-c4ccc(-c5ccccc5)c5oc6c(-c7ccccc7)cccc6c45)c3)cc2)cc1 ; c1ccc(-c2cc(-c3ccccc3)cc(N(c3cccc(-c4ccc(-c5ccccc5)c5oc6c(-c7ccccc7)cccc6c45)c3)c3ccc4c(c3)oc3ccccc34)c2)cc1 ; c1ccc(-c2ccc(N(c3cccc(-c4ccc(-c5ccccc5)c5oc6c(-c7ccccc7)cccc6c45)c3)c3ccc4c(c3)oc3ccccc34)cc2-c2ccccc2)cc1]

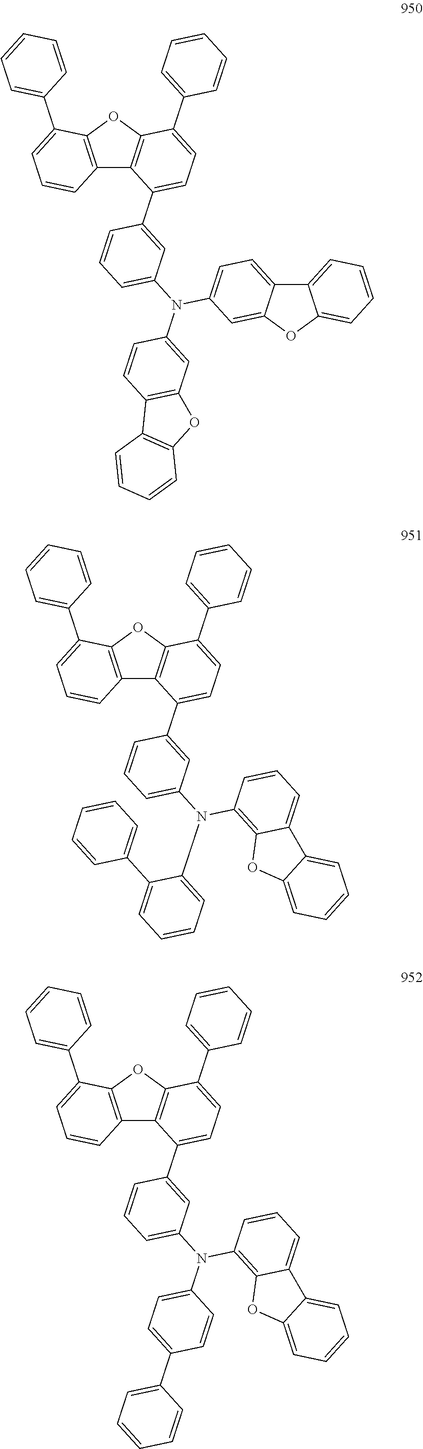 [SMILES: c1ccc(-c2cccc3c2oc2c(-c4ccccc4)ccc(-c4cccc(N(c5ccc6c(c5)oc5ccccc56)c5ccc6c(c5)oc5ccccc56)c4)c23)cc1 ; c1ccc(-c2ccccc2N(c2cccc(-c3ccc(-c4ccccc4)c4oc5c(-c6ccccc6)cccc5c34)c2)c2cccc3c2oc2ccccc23)cc1 ; c1ccc(-c2ccc(N(c3cccc(-c4ccc(-c5ccccc5)c5oc6c(-c7ccccc7)cccc6c45)c3)c3cccc4c3oc3ccccc34)cc2)cc1]

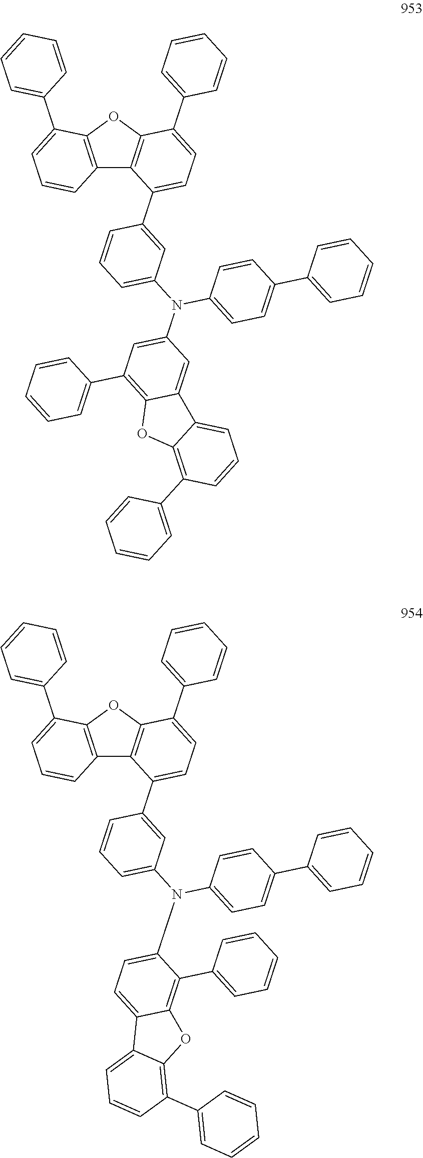 [SMILES: c1ccc(-c2ccc(N(c3cccc(-c4ccc(-c5ccccc5)c5oc6c(-c7ccccc7)cccc6c45)c3)c3cc(-c4ccccc4)c4oc5c(-c6ccccc6)cccc5c4c3)cc2)cc1 ; c1ccc(-c2ccc(N(c3cccc(-c4ccc(-c5ccccc5)c5oc6c(-c7ccccc7)cccc6c45)c3)c3ccc4c(oc5c(-c6ccccc6)cccc54)c3-c3ccccc3)cc2)cc1]

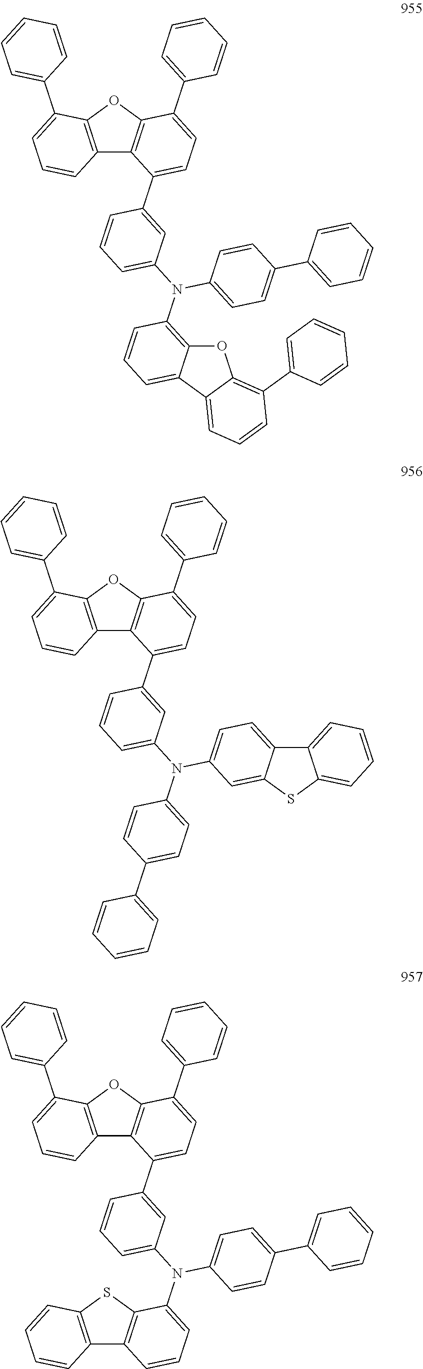 [SMILES: c1ccc(-c2ccc(N(c3cccc(-c4ccc(-c5ccccc5)c5oc6c(-c7ccccc7)cccc6c45)c3)c3cccc4c3oc3c(-c5ccccc5)cccc34)cc2)cc1 ; c1ccc(-c2ccc(N(c3cccc(-c4ccc(-c5ccccc5)c5oc6c(-c7ccccc7)cccc6c45)c3)c3ccc4c(c3)sc3ccccc34)cc2)cc1 ; c1ccc(-c2ccc(N(c3cccc(-c4ccc(-c5ccccc5)c5oc6c(-c7ccccc7)cccc6c45)c3)c3cccc4c3sc3ccccc34)cc2)cc1]

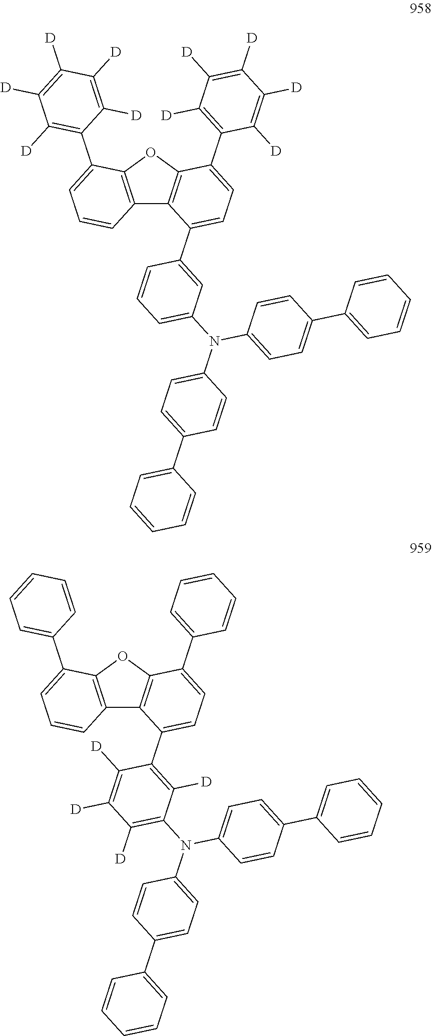 [SMILES: [2H]c1c([2H])c([2H])c(-c2cccc3c2oc2c(-c4c([2H])c([2H])c([2H])c([2H])c4[2H])ccc(-c4cccc(N(c5ccc(-c6ccccc6)cc5)c5ccc(-c6ccccc6)cc5)c4)c23)c([2H])c1[2H] ; [2H]c1c([2H])c(-c2ccc(-c3ccccc3)c3oc4c(-c5ccccc5)cccc4c23)c([2H])c(N(c2ccc(-c3ccccc3)cc2)c2ccc(-c3ccccc3)cc2)c1[2H]]

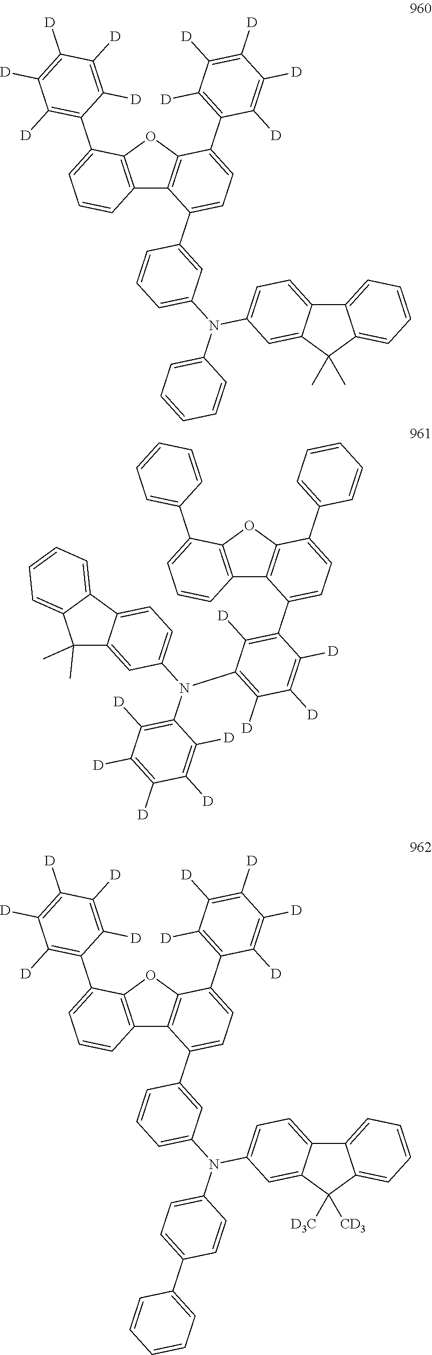

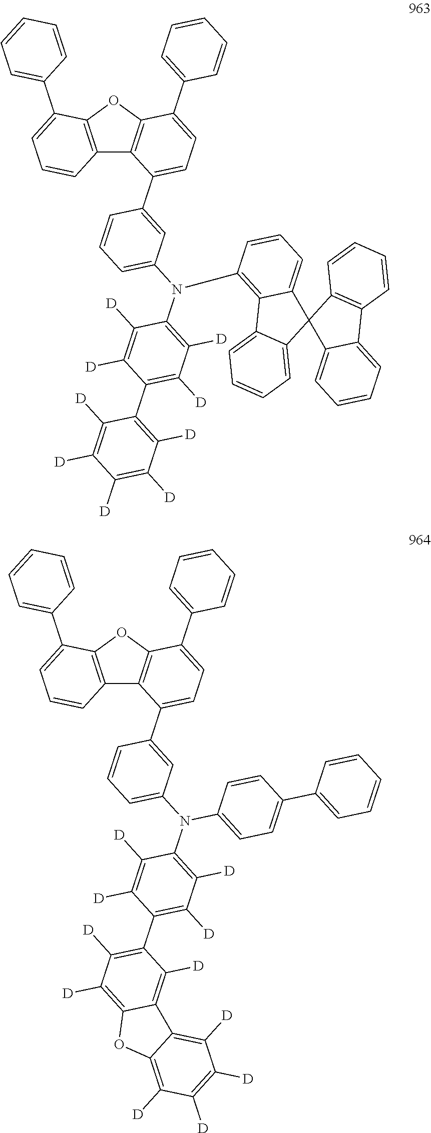 [SMILES: [2H]c1c([2H])c([2H])c(-c2c([2H])c([2H])c(N(c3cccc(-c4ccc(-c5ccccc5)c5oc6c(-c7ccccc7)cccc6c45)c3)c3cccc4c3-c3ccccc3C43c4ccccc4-c4ccccc43)c([2H])c2[2H])c([2H])c1[2H] ; [2H]c1c([2H])c(N(c2ccc(-c3ccccc3)cc2)c2cccc(-c3ccc(-c4ccccc4)c4oc5c(-c6ccccc6)cccc5c34)c2)c([2H])c([2H])c1-c1c([2H])c([2H])c2oc3c([2H])c([2H])c([2H])c([2H])c3c2c1[2H]]

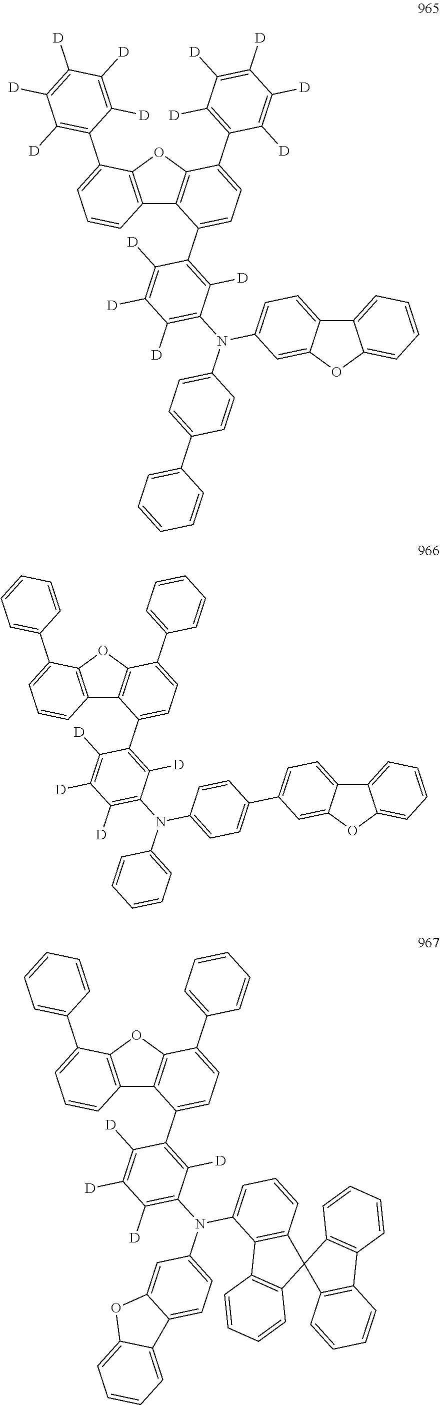 [SMILES: [2H]c1c([2H])c([2H])c(-c2cccc3c2oc2c(-c4c([2H])c([2H])c([2H])c([2H])c4[2H])ccc(-c4c([2H])c([2H])c([2H])c(N(c5ccc(-c6ccccc6)cc5)c5ccc6c(c5)oc5ccccc56)c4[2H])c23)c([2H])c1[2H] ; [2H]c1c([2H])c(-c2ccc(-c3ccccc3)c3oc4c(-c5ccccc5)cccc4c23)c([2H])c(N(c2ccccc2)c2ccc(-c3ccc4c(c3)oc3ccccc34)cc2)c1[2H] ; [2H]c1c([2H])c(-c2ccc(-c3ccccc3)c3oc4c(-c5ccccc5)cccc4c23)c([2H])c(N(c2ccc3c(c2)oc2ccccc23)c2cccc3c2-c2ccccc2C32c3ccccc3-c3ccccc32)c1[2H]]

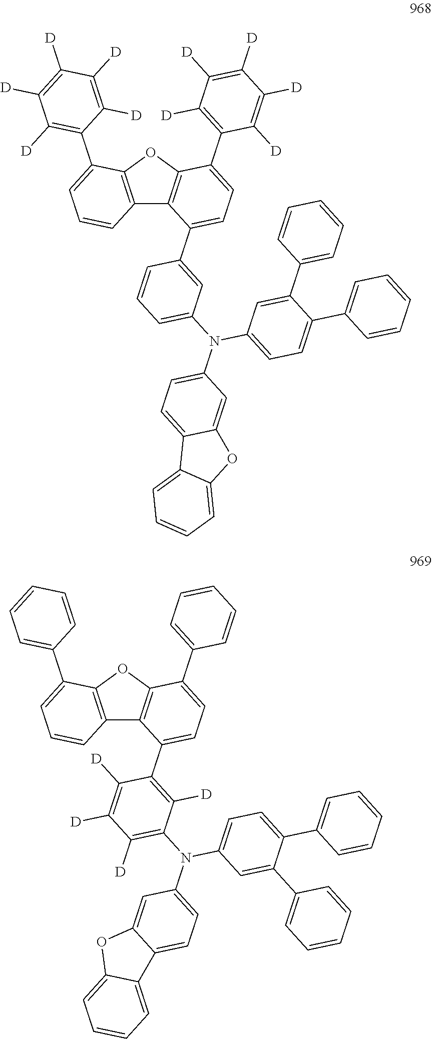 [SMILES: [2H]c1c([2H])c([2H])c(-c2cccc3c2oc2c(-c4c([2H])c([2H])c([2H])c([2H])c4[2H])ccc(-c4cccc(N(c5ccc(-c6ccccc6)c(-c6ccccc6)c5)c5ccc6c(c5)oc5ccccc56)c4)c23)c([2H])c1[2H] ; [2H]c1c([2H])c(-c2ccc(-c3ccccc3)c3oc4c(-c5ccccc5)cccc4c23)c([2H])c(N(c2ccc(-c3ccccc3)c(-c3ccccc3)c2)c2ccc3c(c2)oc2ccccc23)c1[2H]]

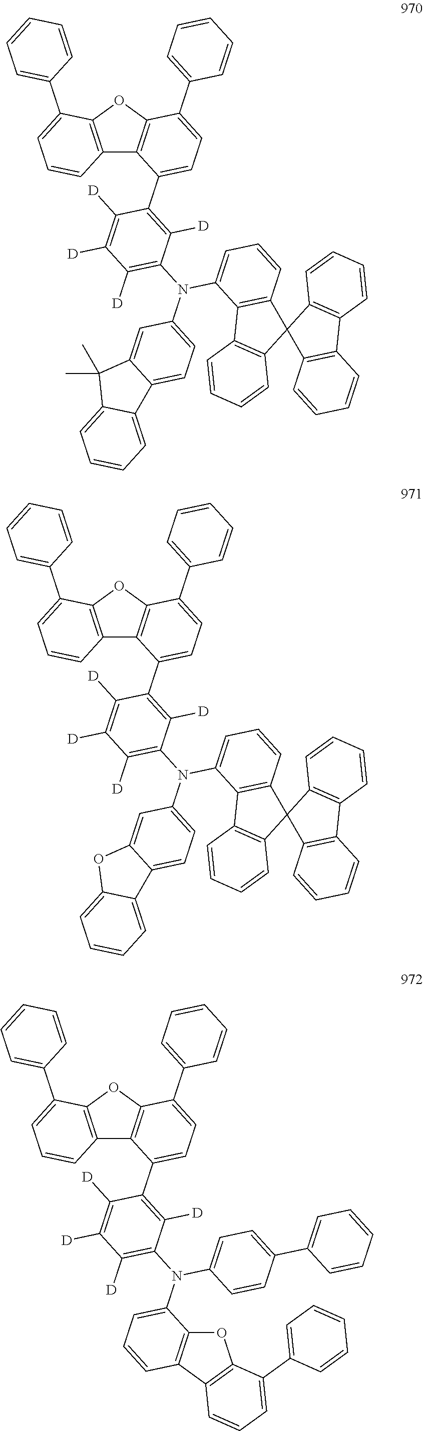 [SMILES: [2H]c1c([2H])c(-c2ccc(-c3ccccc3)c3oc4c(-c5ccccc5)cccc4c23)c([2H])c(N(c2ccc3c(c2)C(C)(C)c2ccccc2-3)c2cccc3c2-c2ccccc2C32c3ccccc3-c3ccccc32)c1[2H] ; [2H]c1c([2H])c(-c2ccc(-c3ccccc3)c3oc4c(-c5ccccc5)cccc4c23)c([2H])c(N(c2ccc3c(c2)oc2ccccc23)c2cccc3c2-c2ccccc2C32c3ccccc3-c3ccccc32)c1[2H] ; [2H]c1c([2H])c(-c2ccc(-c3ccccc3)c3oc4c(-c5ccccc5)cccc4c23)c([2H])c(N(c2ccc(-c3ccccc3)cc2)c2cccc3c2oc2c(-c4ccccc4)cccc23)c1[2H]]

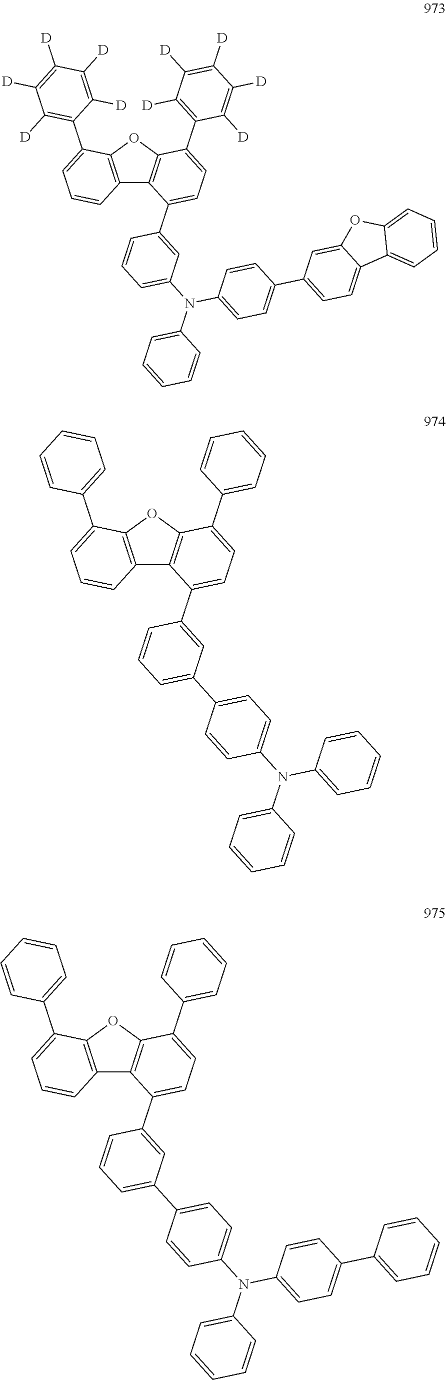 [SMILES: [2H]c1c([2H])c([2H])c(-c2cccc3c2oc2c(-c4c([2H])c([2H])c([2H])c([2H])c4[2H])ccc(-c4cccc(N(c5ccccc5)c5ccc(-c6ccc7c(c6)oc6ccccc67)cc5)c4)c23)c([2H])c1[2H] ; c1ccc(-c2cccc3c2oc2c(-c4ccccc4)ccc(-c4cccc(-c5ccc(N(c6ccccc6)c6ccccc6)cc5)c4)c23)cc1 ; c1ccc(-c2ccc(N(c3ccccc3)c3ccc(-c4cccc(-c5ccc(-c6ccccc6)c6oc7c(-c8ccccc8)cccc7c56)c4)cc3)cc2)cc1]

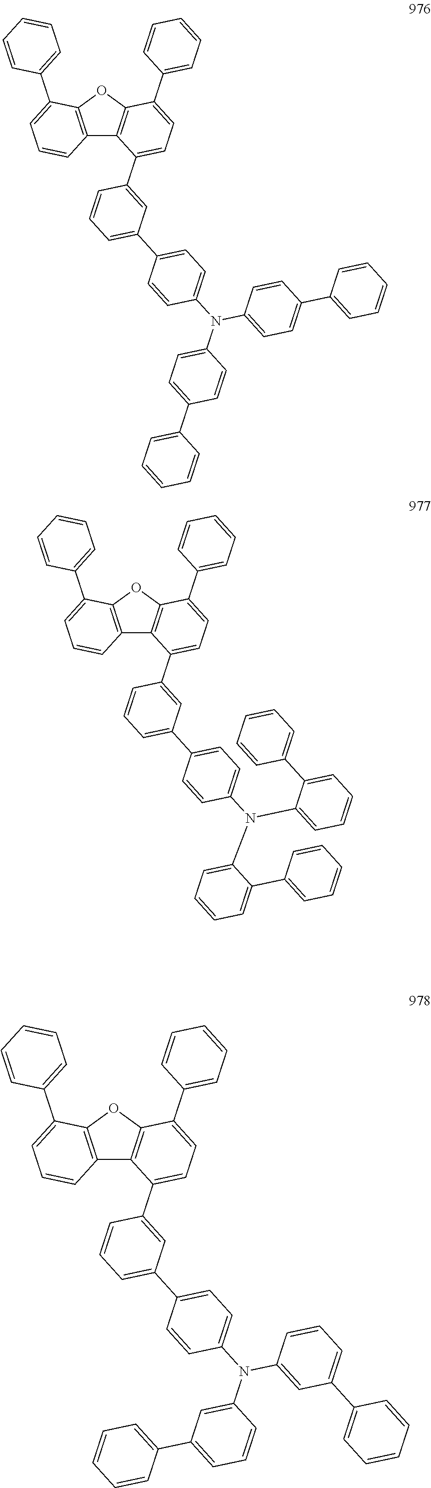

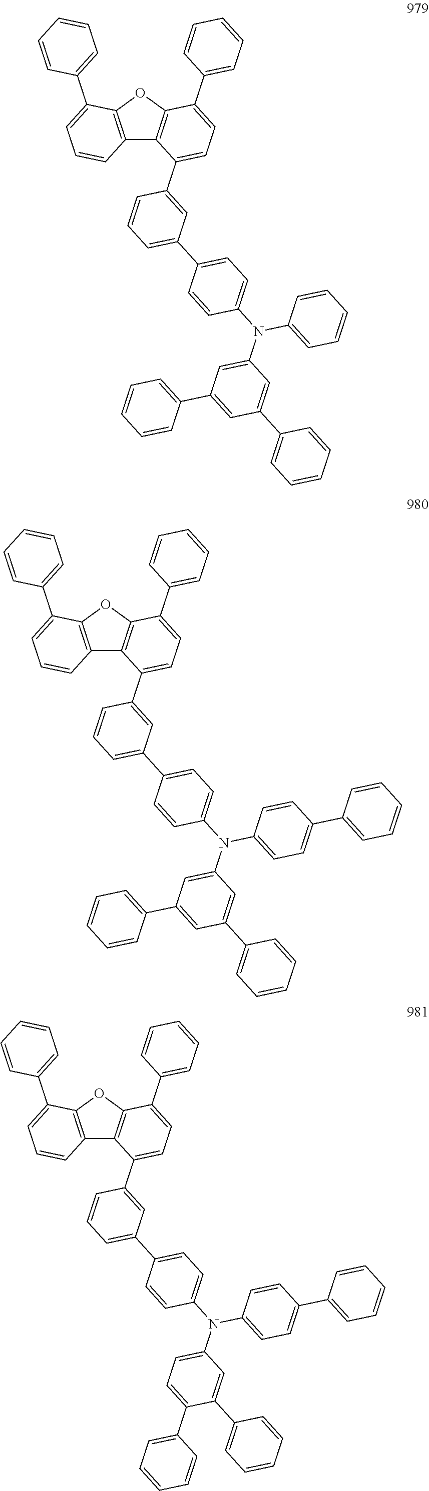 [SMILES: c1ccc(-c2cc(-c3ccccc3)cc(N(c3ccccc3)c3ccc(-c4cccc(-c5ccc(-c6ccccc6)c6oc7c(-c8ccccc8)cccc7c56)c4)cc3)c2)cc1 ; c1ccc(-c2ccc(N(c3ccc(-c4cccc(-c5ccc(-c6ccccc6)c6oc7c(-c8ccccc8)cccc7c56)c4)cc3)c3cc(-c4ccccc4)cc(-c4ccccc4)c3)cc2)cc1 ; c1ccc(-c2ccc(N(c3ccc(-c4cccc(-c5ccc(-c6ccccc6)c6oc7c(-c8ccccc8)cccc7c56)c4)cc3)c3ccc(-c4ccccc4)c(-c4ccccc4)c3)cc2)cc1]

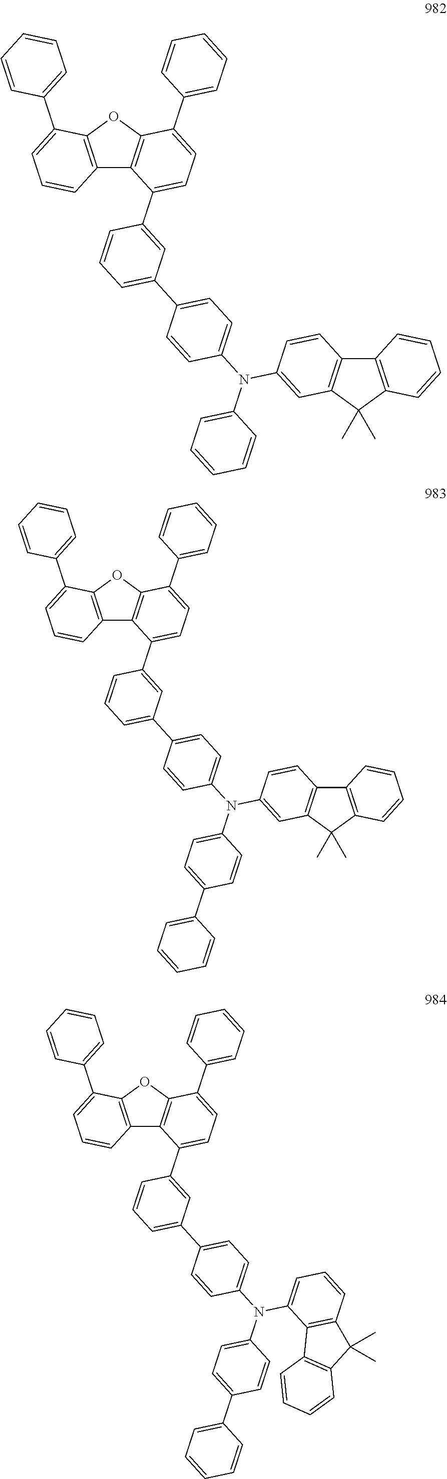

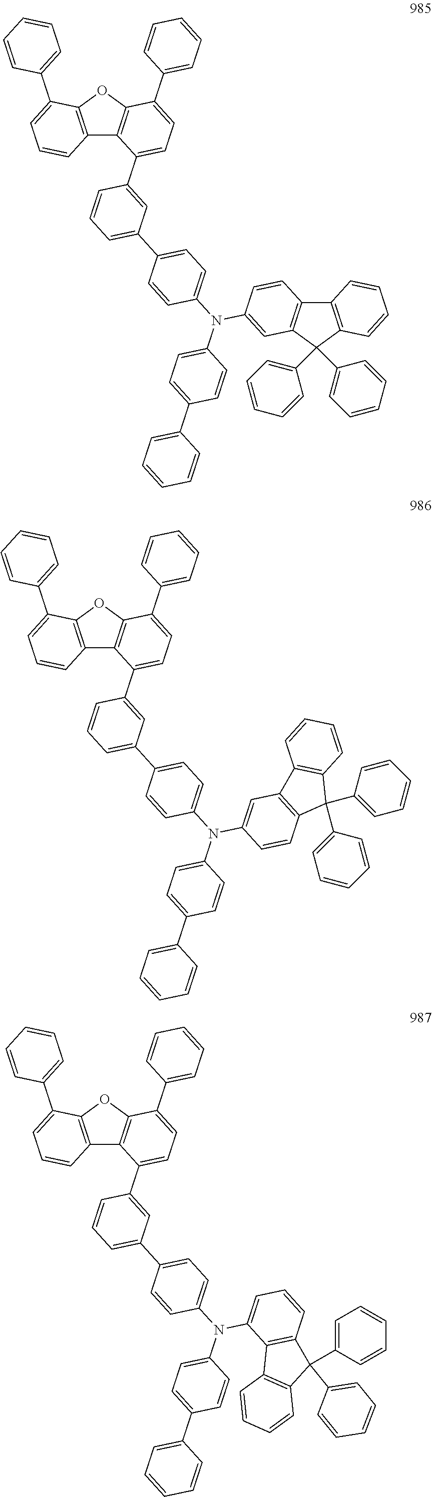

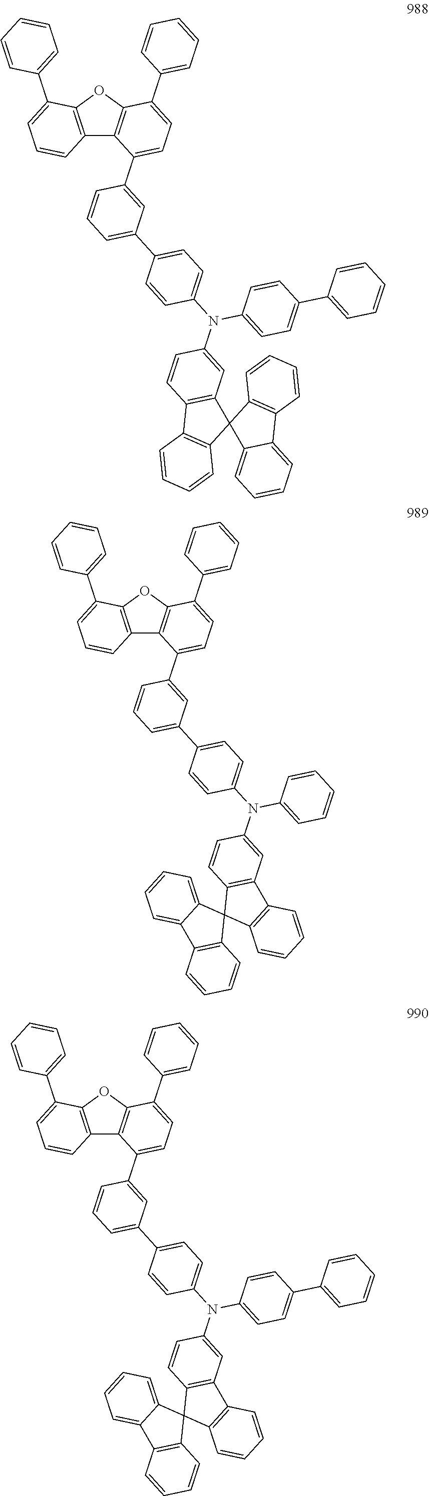

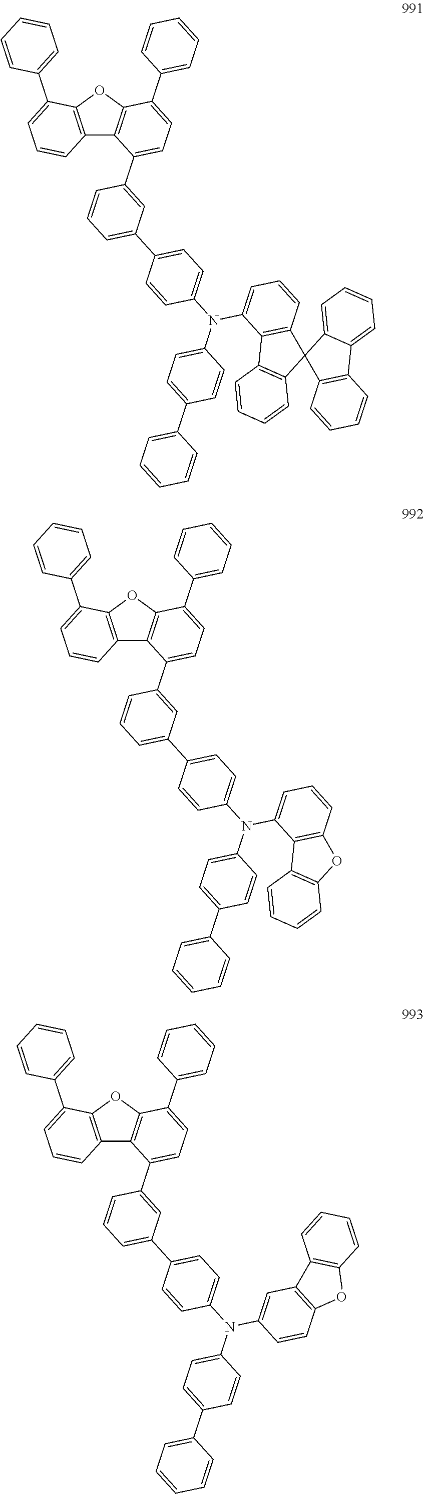

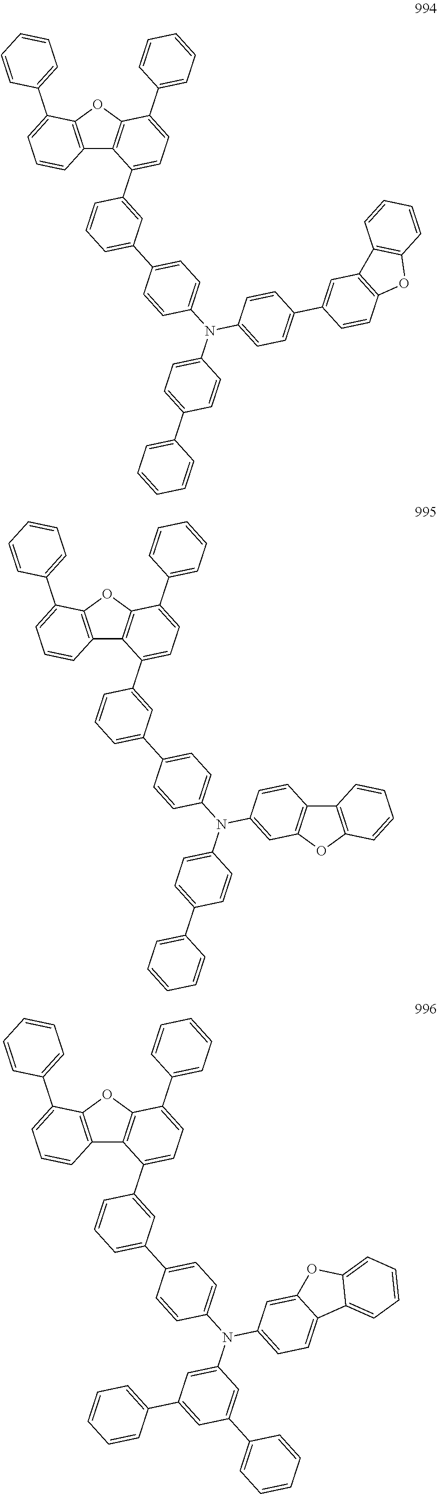

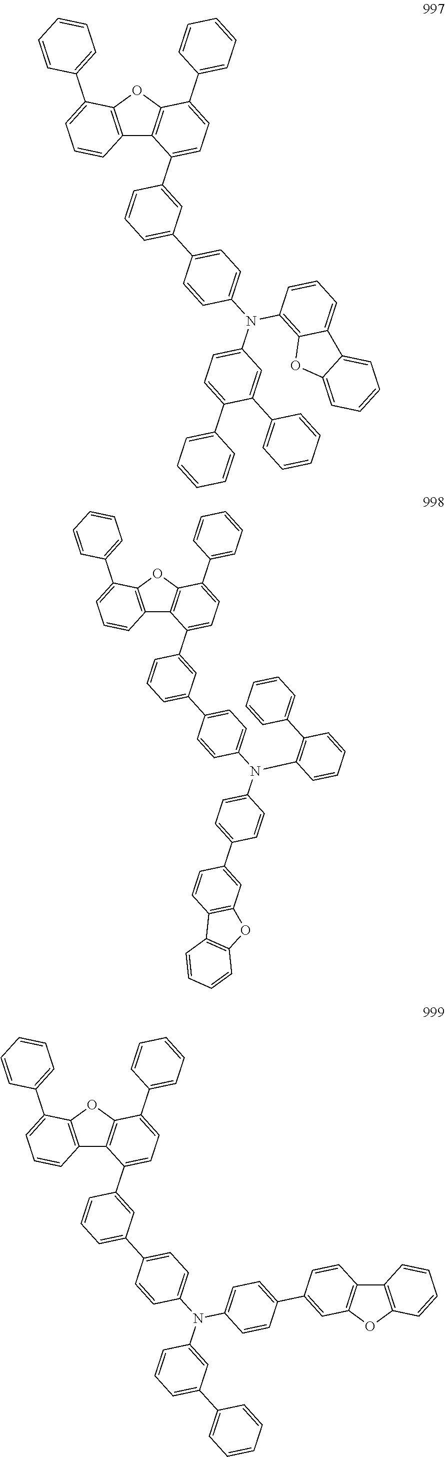

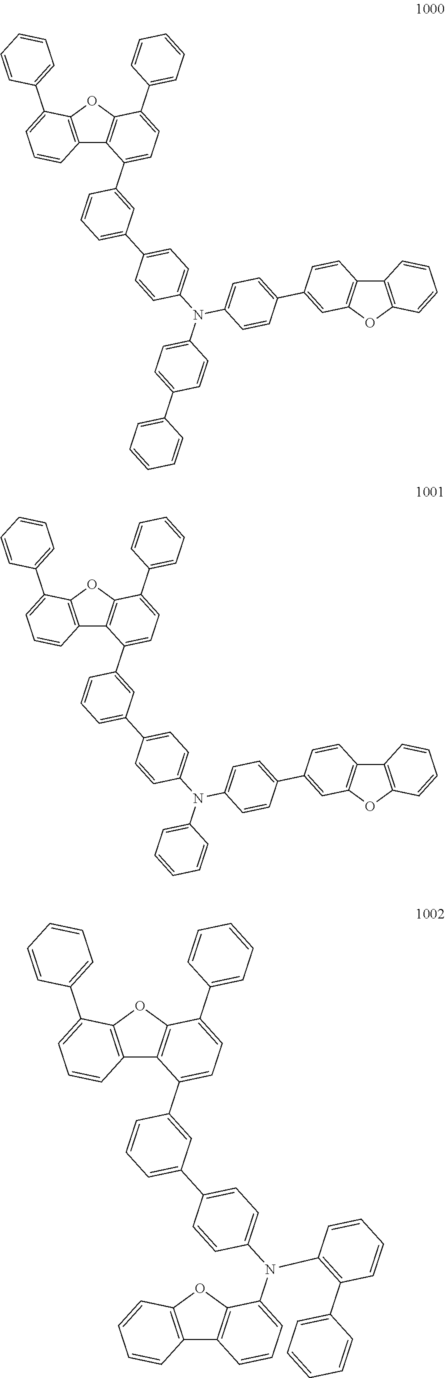 [SMILES: c1ccc(-c2ccc(N(c3ccc(-c4cccc(-c5ccc(-c6ccccc6)c6oc7c(-c8ccccc8)cccc7c56)c4)cc3)c3ccc(-c4ccc5c(c4)oc4ccccc45)cc3)cc2)cc1 ; c1ccc(-c2cccc3c2oc2c(-c4ccccc4)ccc(-c4cccc(-c5ccc(N(c6ccccc6)c6ccc(-c7ccc8c(c7)oc7ccccc78)cc6)cc5)c4)c23)cc1 ; c1ccc(-c2ccccc2N(c2ccc(-c3cccc(-c4ccc(-c5ccccc5)c5oc6c(-c7ccccc7)cccc6c45)c3)cc2)c2cccc3c2oc2ccccc23)cc1]

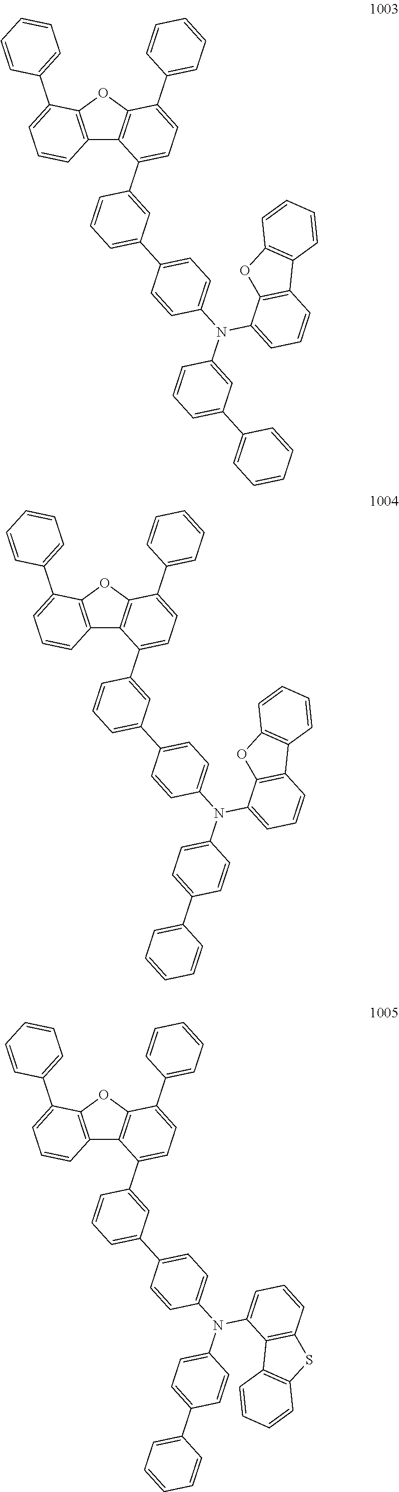 [SMILES: c1ccc(-c2cccc(N(c3ccc(-c4cccc(-c5ccc(-c6ccccc6)c6oc7c(-c8ccccc8)cccc7c56)c4)cc3)c3cccc4c3oc3ccccc34)c2)cc1 ; c1ccc(-c2ccc(N(c3ccc(-c4cccc(-c5ccc(-c6ccccc6)c6oc7c(-c8ccccc8)cccc7c56)c4)cc3)c3cccc4c3oc3ccccc34)cc2)cc1 ; c1ccc(-c2ccc(N(c3ccc(-c4cccc(-c5ccc(-c6ccccc6)c6oc7c(-c8ccccc8)cccc7c56)c4)cc3)c3cccc4sc5ccccc5c34)cc2)cc1]

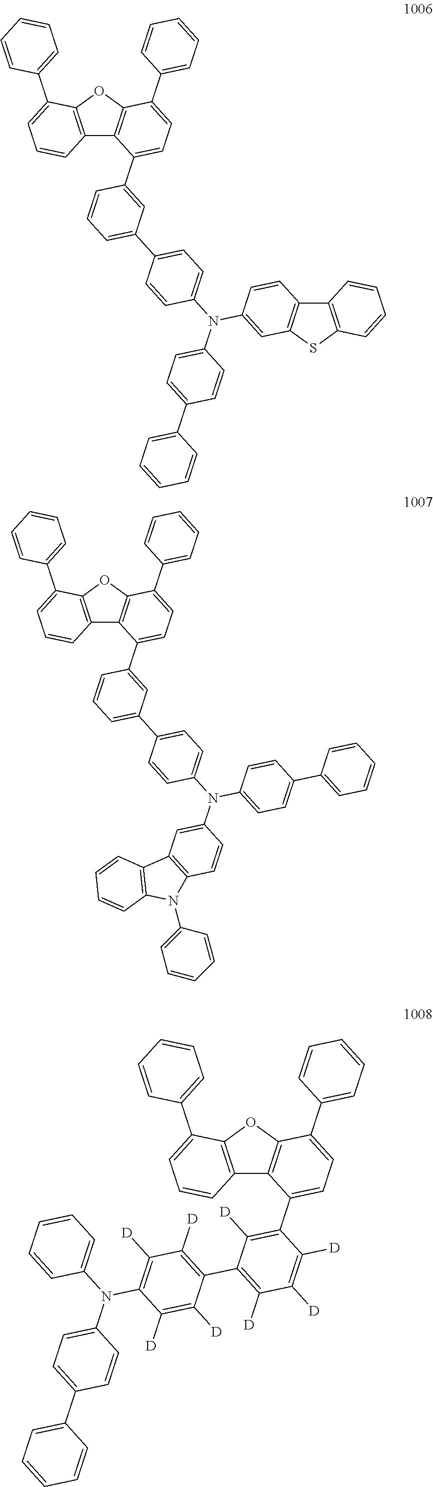

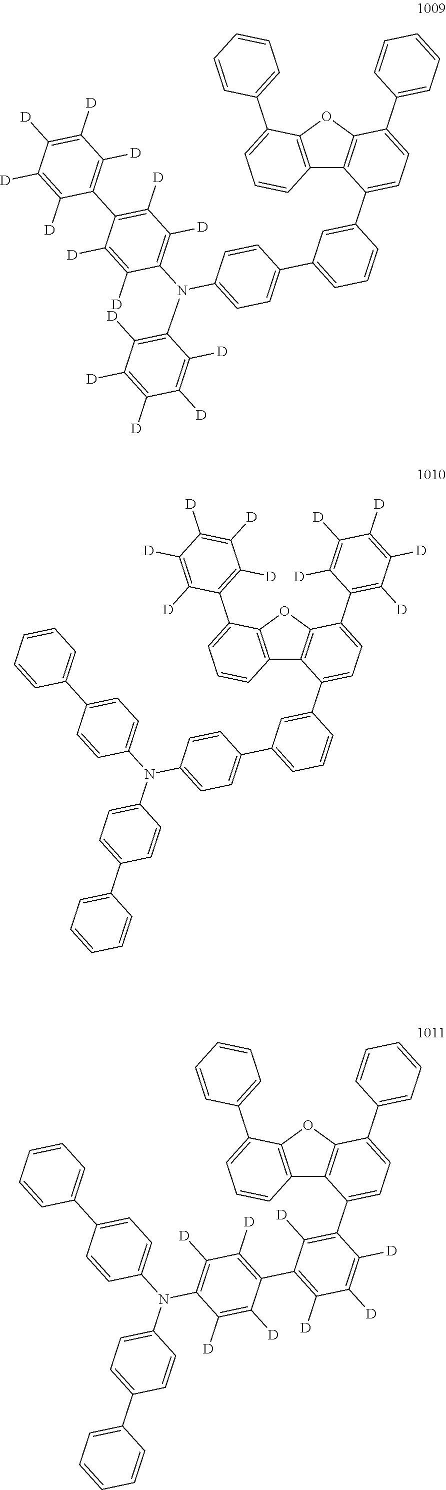 [SMILES: [2H]c1c([2H])c([2H])c(-c2c([2H])c([2H])c(N(c3ccc(-c4cccc(-c5ccc(-c6ccccc6)c6oc7c(-c8ccccc8)cccc7c56)c4)cc3)c3c([2H])c([2H])c([2H])c([2H])c3[2H])c([2H])c2[2H])c([2H])c1[2H] ; [2H]c1c([2H])c([2H])c(-c2cccc3c2oc2c(-c4c([2H])c([2H])c([2H])c([2H])c4[2H])ccc(-c4cccc(-c5ccc(N(c6ccc(-c7ccccc7)cc6)c6ccc(-c7ccccc7)cc6)cc5)c4)c23)c([2H])c1[2H] ; [2H]c1c([2H])c(-c2c([2H])c([2H])c(N(c3ccc(-c4ccccc4)cc3)c3ccc(-c4ccccc4)cc3)c([2H])c2[2H])c([2H])c(-c2ccc(-c3ccccc3)c3oc4c(-c5ccccc5)cccc4c23)c1[2H]]

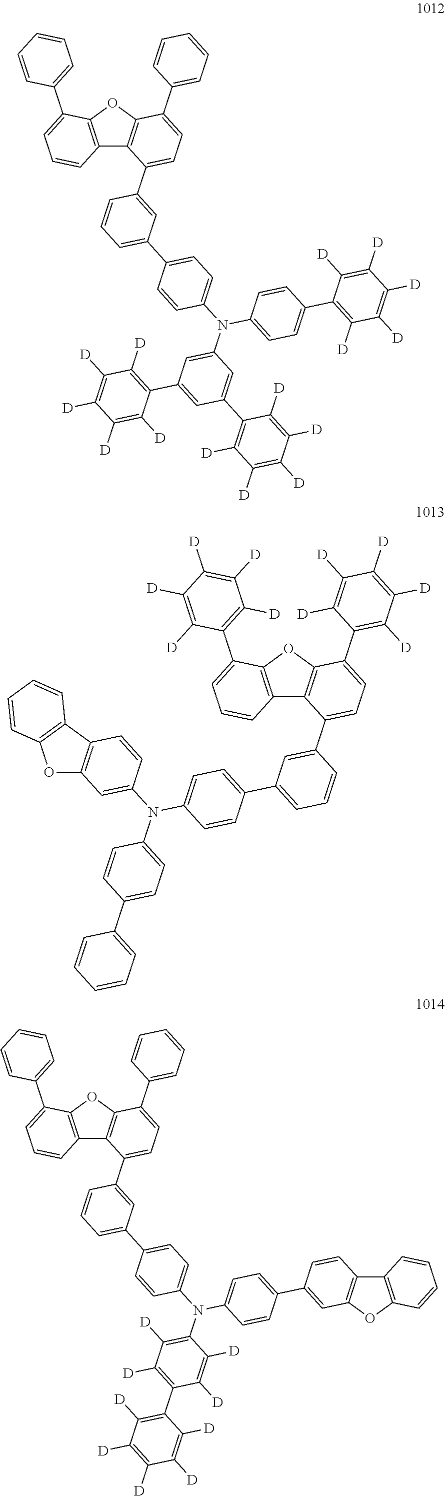

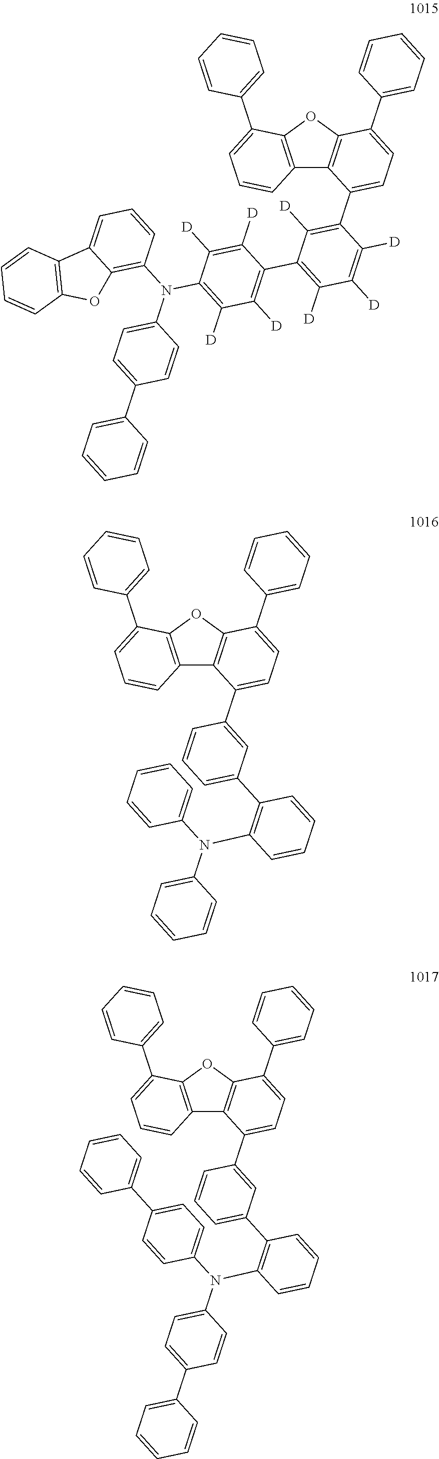 [SMILES: [2H]c1c([2H])c(-c2c([2H])c([2H])c(N(c3ccc(-c4ccccc4)cc3)c3cccc4c3oc3ccccc34)c([2H])c2[2H])c([2H])c(-c2ccc(-c3ccccc3)c3oc4c(-c5ccccc5)cccc4c23)c1[2H] ; c1ccc(-c2cccc3c2oc2c(-c4ccccc4)ccc(-c4cccc(-c5ccccc5N(c5ccccc5)c5ccccc5)c4)c23)cc1 ; c1ccc(-c2ccc(N(c3ccc(-c4ccccc4)cc3)c3ccccc3-c3cccc(-c4ccc(-c5ccccc5)c5oc6c(-c7ccccc7)cccc6c45)c3)cc2)cc1]

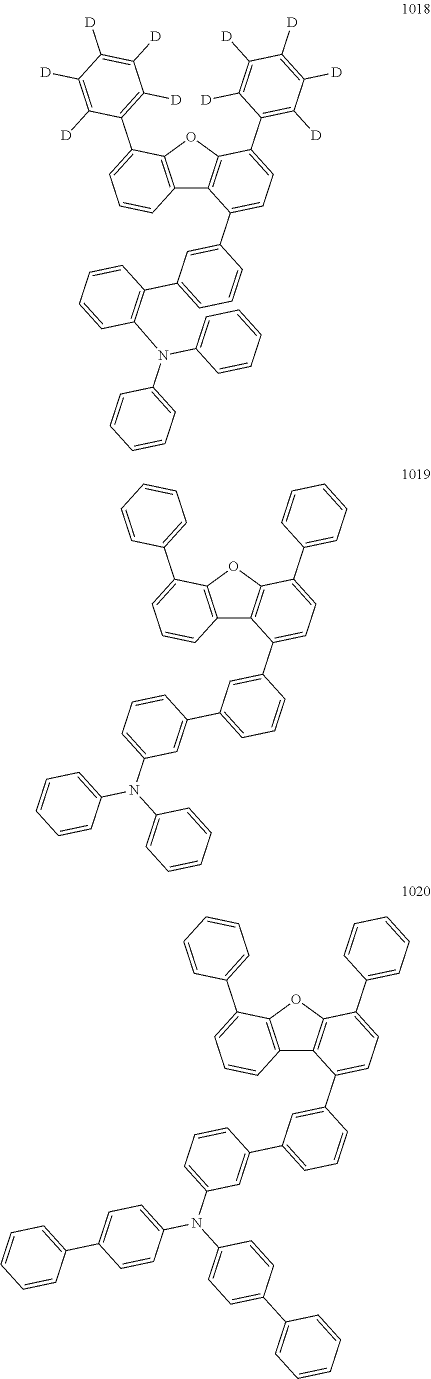

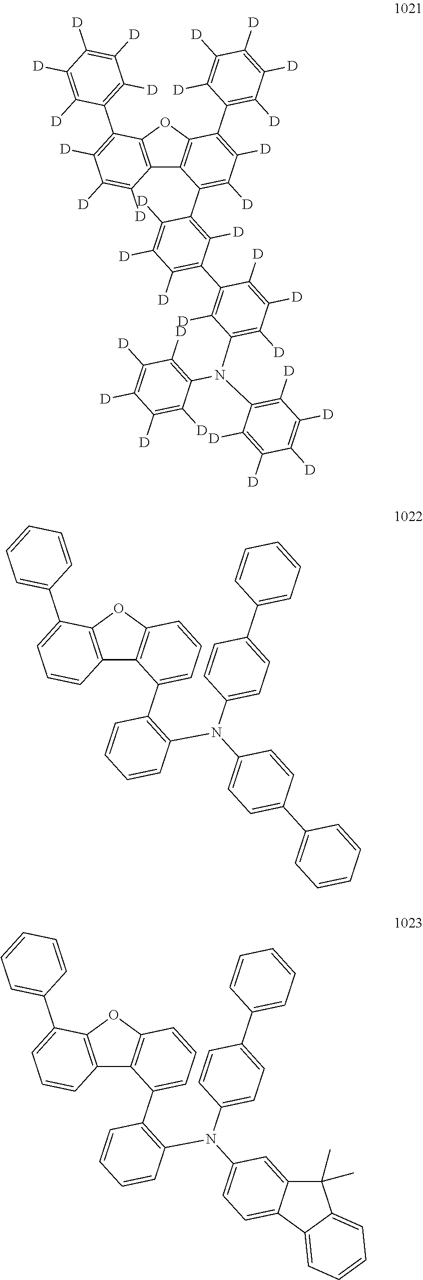 [SMILES: [2H]c1c([2H])c([2H])c(-c2c([2H])c([2H])c([2H])c3c2oc2c(-c4c([2H])c([2H])c([2H])c([2H])c4[2H])c([2H])c([2H])c(-c4c([2H])c([2H])c([2H])c(-c5c([2H])c([2H])c([2H])c(N(c6c([2H])c([2H])c([2H])c([2H])c6[2H])c6c([2H])c([2H])c([2H])c([2H])c6[2H])c5[2H])c4[2H])c23)c([2H])c1[2H] ; c1ccc(-c2ccc(N(c3ccc(-c4ccccc4)cc3)c3ccccc3-c3cccc4oc5c(-c6ccccc6)cccc5c34)cc2)cc1 ; CC1(C)c2ccccc2-c2ccc(N(c3ccc(-c4ccccc4)cc3)c3ccccc3-c3cccc4oc5c(-c6ccccc6)cccc5c34)cc21]

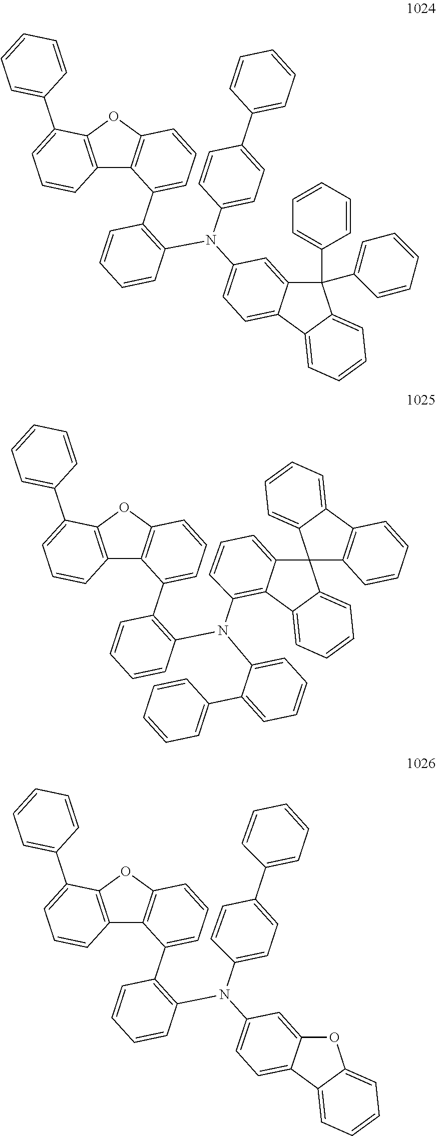 [SMILES: c1ccc(-c2ccc(N(c3ccc4c(c3)C(c3ccccc3)(c3ccccc3)c3ccccc3-4)c3ccccc3-c3cccc4oc5c(-c6ccccc6)cccc5c34)cc2)cc1 ; c1ccc(-c2ccccc2N(c2ccccc2-c2cccc3oc4c(-c5ccccc5)cccc4c23)c2cccc3c2-c2ccccc2C32c3ccccc3-c3ccccc32)cc1 ; c1ccc(-c2ccc(N(c3ccc4c(c3)oc3ccccc34)c3ccccc3-c3cccc4oc5c(-c6ccccc6)cccc5c34)cc2)cc1]

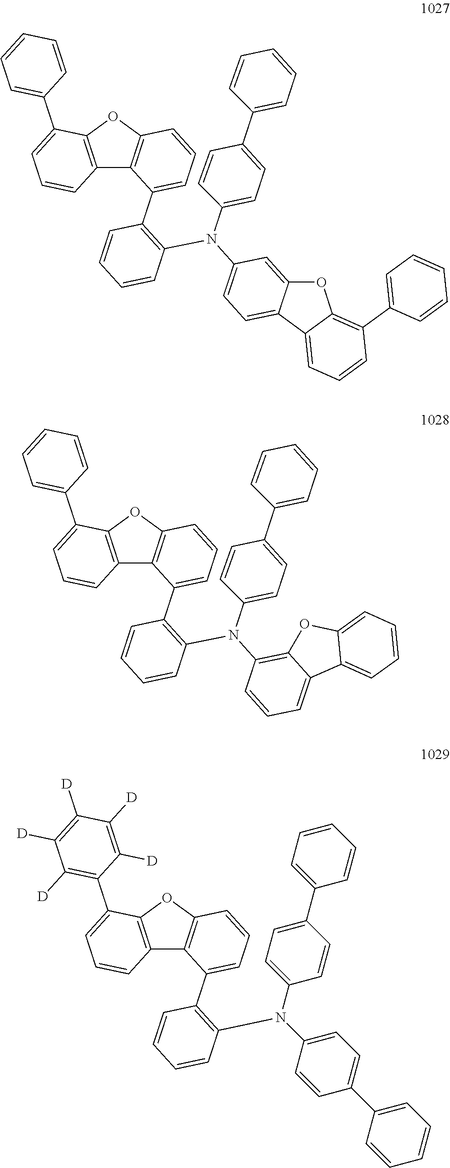 [SMILES: c1ccc(-c2ccc(N(c3ccc4c(c3)oc3c(-c5ccccc5)cccc34)c3ccccc3-c3cccc4oc5c(-c6ccccc6)cccc5c34)cc2)cc1 ; c1ccc(-c2ccc(N(c3ccccc3-c3cccc4oc5c(-c6ccccc6)cccc5c34)c3cccc4c3oc3ccccc34)cc2)cc1 ; [2H]c1c([2H])c([2H])c(-c2cccc3c2oc2cccc(-c4ccccc4N(c4ccc(-c5ccccc5)cc4)c4ccc(-c5ccccc5)cc4)c23)c([2H])c1[2H]]

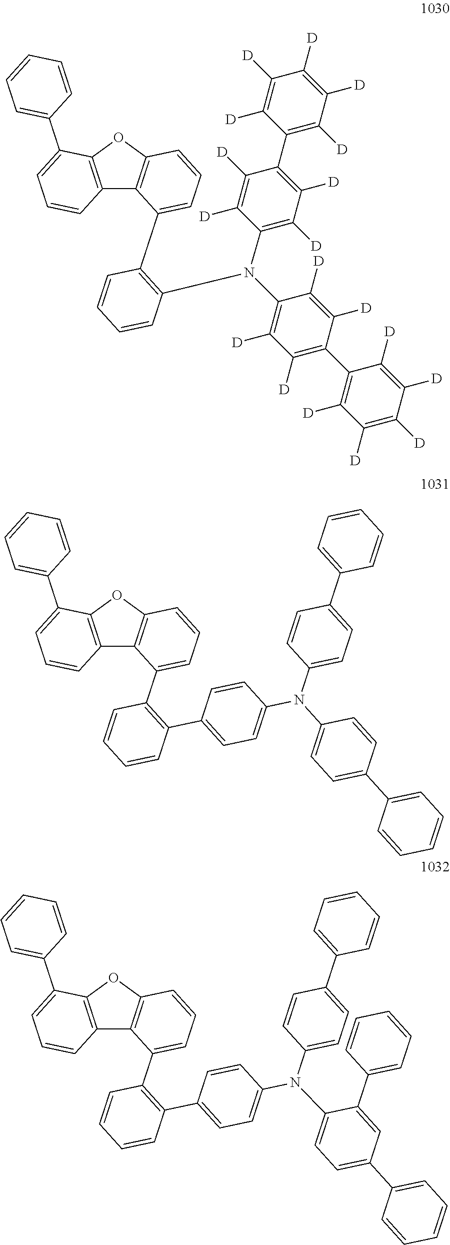 [SMILES: [2H]c1c([2H])c([2H])c(-c2c([2H])c([2H])c(N(c3ccccc3-c3cccc4oc5c(-c6ccccc6)cccc5c34)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3ccc(-c4ccccc4)cc3)c3ccc(-c4ccccc4-c4cccc5oc6c(-c7ccccc7)cccc6c45)cc3)cc2)cc1 ; c1ccc(-c2ccc(N(c3ccc(-c4ccccc4-c4cccc5oc6c(-c7ccccc7)cccc6c45)cc3)c3ccc(-c4ccccc4)cc3-c3ccccc3)cc2)cc1]

[chemical structure]

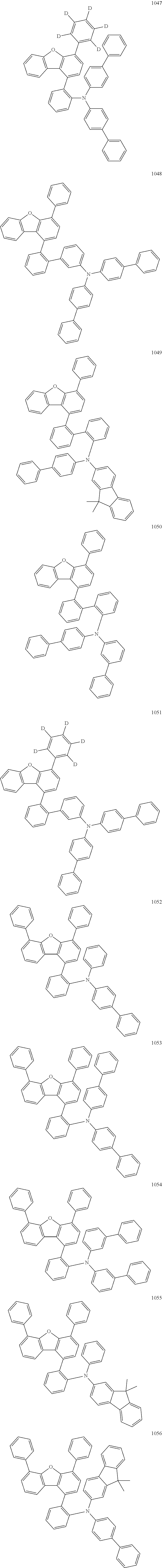

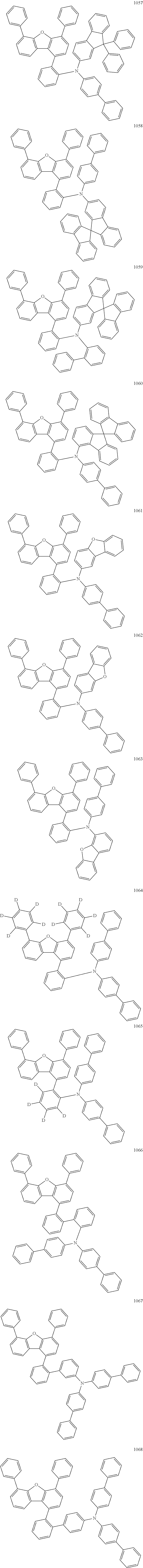

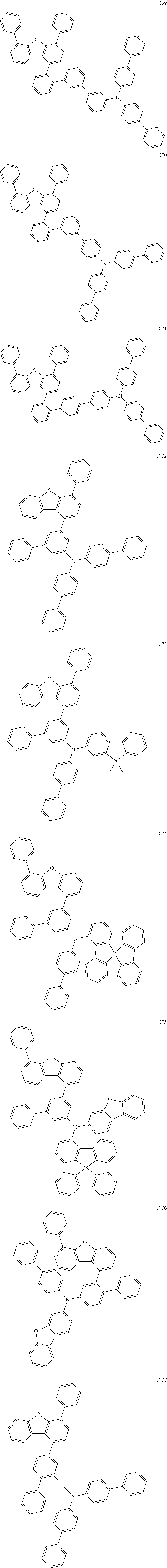

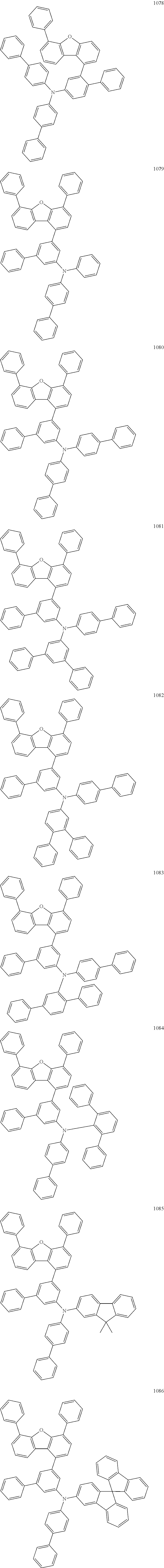

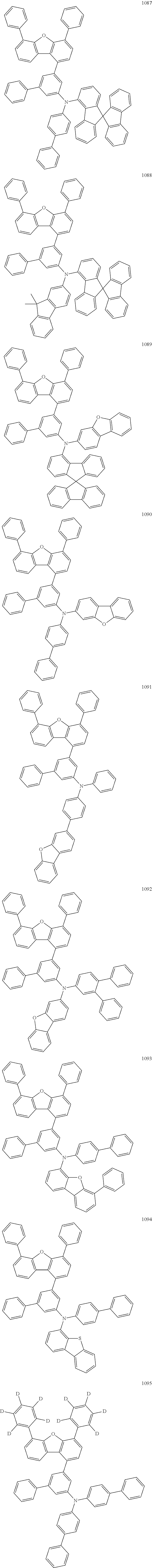

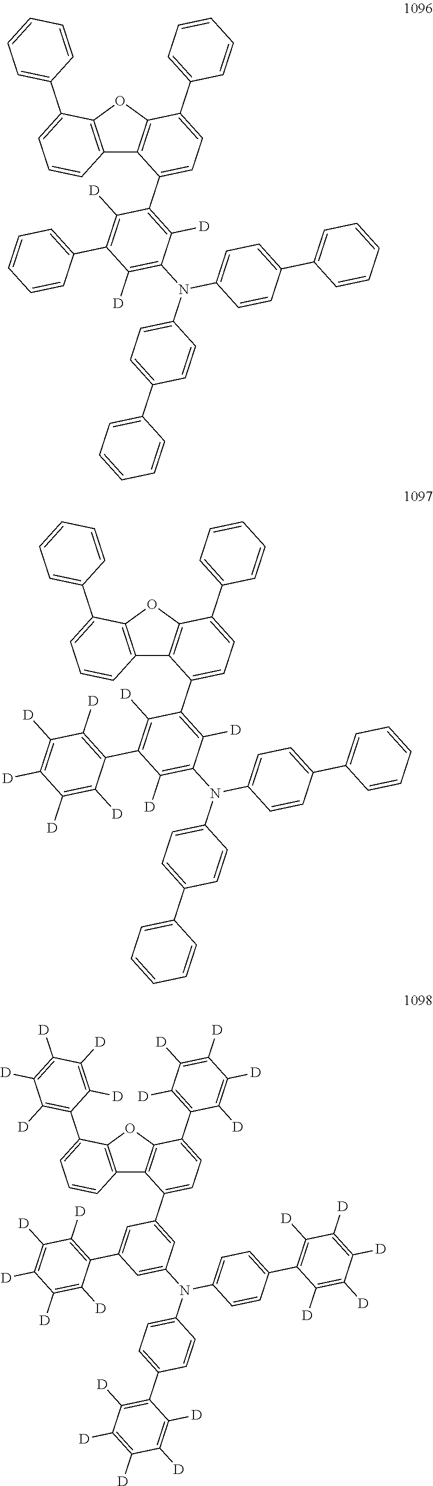

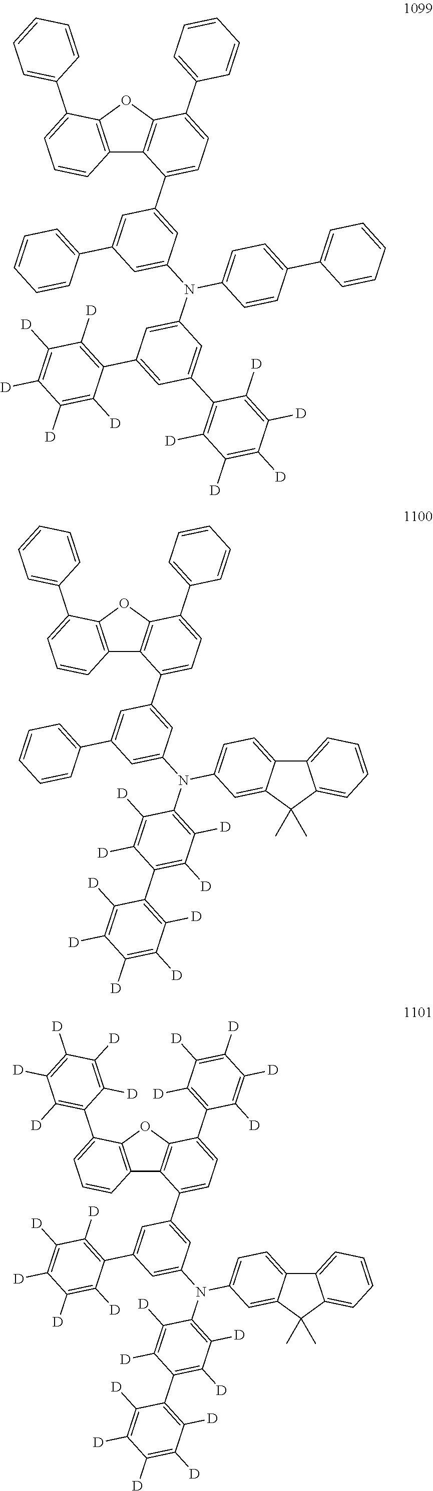

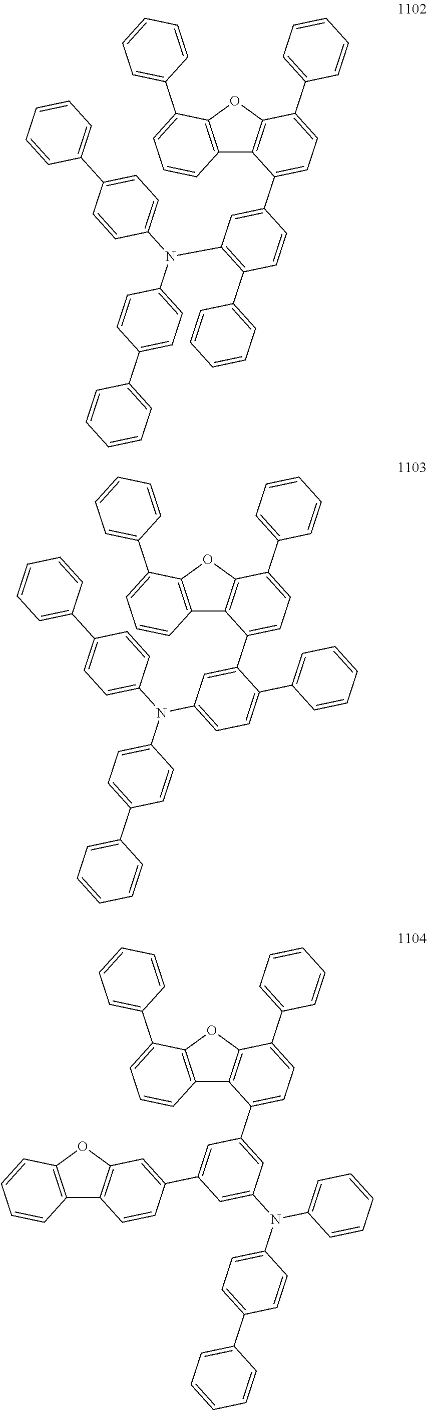

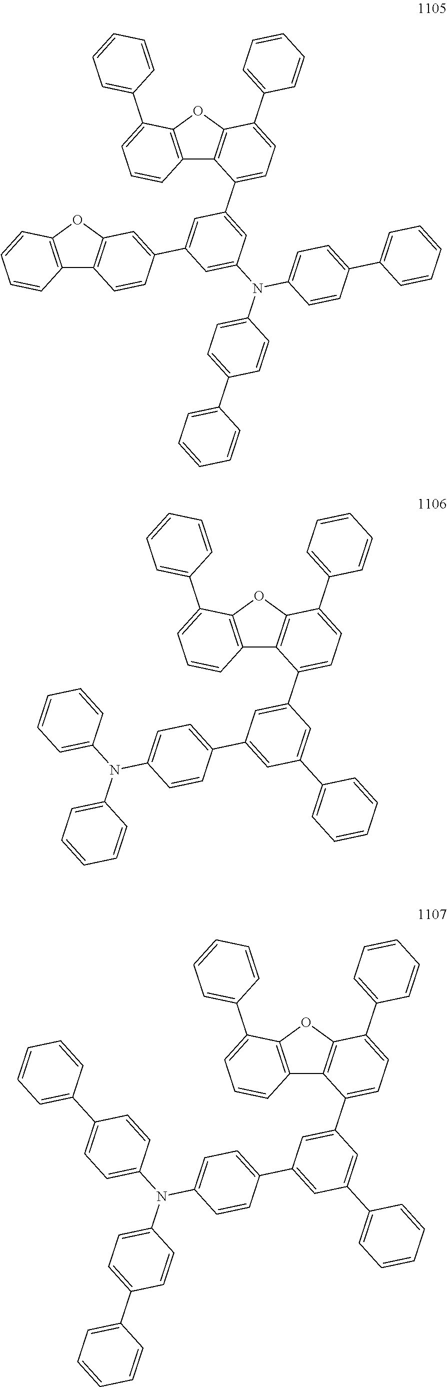 [SMILES: c1ccc(-c2ccc(N(c3ccc(-c4ccccc4)cc3)c3cc(-c4ccc5c(c4)oc4ccccc45)cc(-c4ccc(-c5ccccc5)c5oc6c(-c7ccccc7)cccc6c45)c3)cc2)cc1 ; c1ccc(-c2cc(-c3ccc(N(c4ccccc4)c4ccccc4)cc3)cc(-c3ccc(-c4ccccc4)c4oc5c(-c6ccccc6)cccc5c34)c2)cc1 ; c1ccc(-c2ccc(N(c3ccc(-c4ccccc4)cc3)c3ccc(-c4cc(-c5ccccc5)cc(-c5ccc(-c6ccccc6)c6oc7c(-c8ccccc8)cccc7c56)c4)cc3)cc2)cc1]

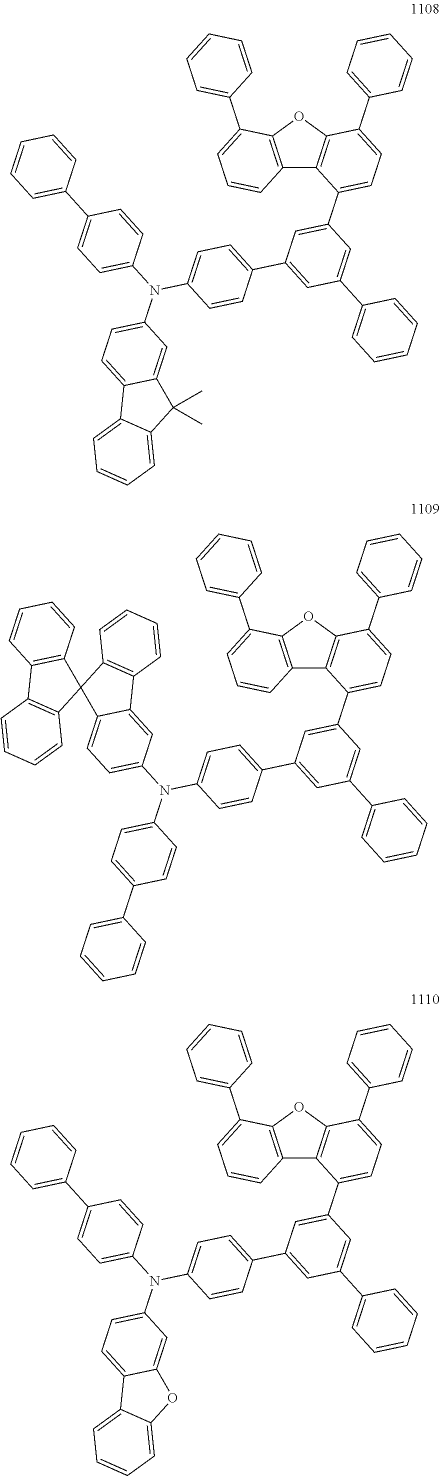 [SMILES: CC1(C)c2ccccc2-c2ccc(N(c3ccc(-c4ccccc4)cc3)c3ccc(-c4cc(-c5ccccc5)cc(-c5ccc(-c6ccccc6)c6oc7c(-c8ccccc8)cccc7c56)c4)cc3)cc21 ; c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc4c(c3)-c3ccccc3C43c4ccccc4-c4ccccc43)cc2)cc1 ; c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc4c(c3)oc3ccccc34)cc2)cc1]

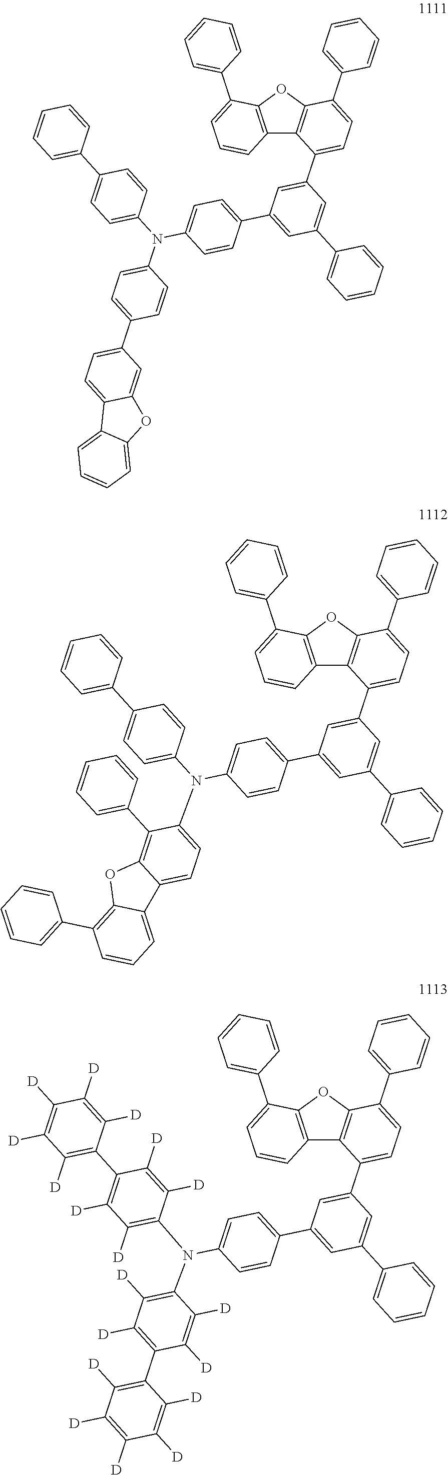 [SMILES: c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc(-c4ccc5c(c4)oc4ccccc45)cc3)cc2)cc1 ; c1ccc(-c2ccc(N(c3ccc(-c4cc(-c5ccccc5)cc(-c5ccc(-c6ccccc6)c6oc7c(-c8ccccc8)cccc7c56)c4)cc3)c3ccc4c(oc5c(-c6ccccc6)cccc54)c3-c3ccccc3)cc2)cc1 ; [2H]c1c([2H])c([2H])c(-c2c([2H])c([2H])c(N(c3ccc(-c4cc(-c5ccccc5)cc(-c5ccc(-c6ccccc6)c6oc7c(-c8ccccc8)cccc7c56)c4)cc3)c3c([2H])c([2H])c(-c4c([2H])c([2H])c([2H])c([2H])c4[2H])c([2H])c3[2H])c([2H])c2[2H])c([2H])c1[2H]]

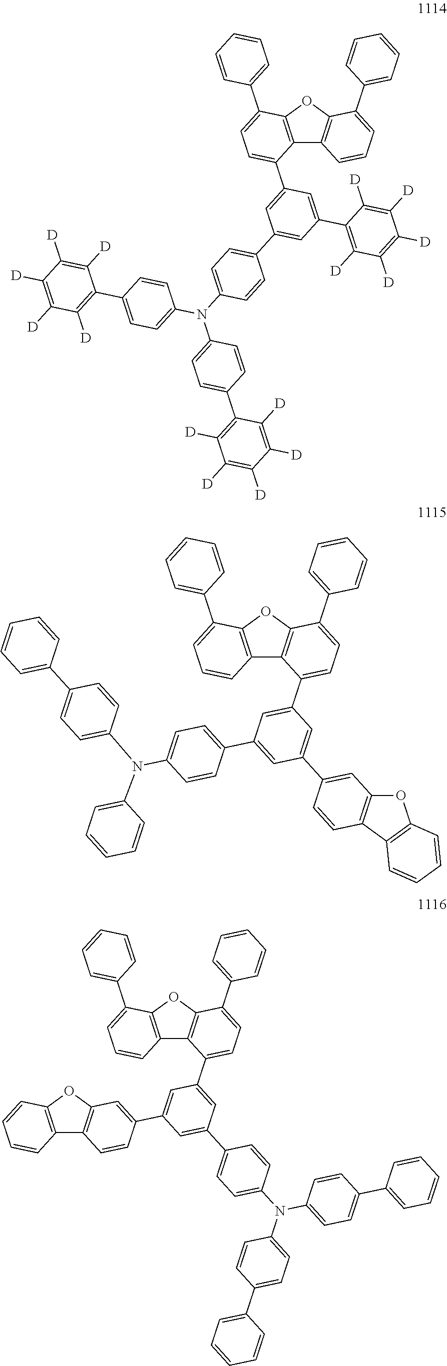 [SMILES: [2H]c1c([2H])c([2H])c(-c2ccc(N(c3ccc(-c4cc(-c5c([2H])c([2H])c([2H])c([2H])c5[2H])cc(-c5ccc(-c6ccccc6)c6oc7c(-c8ccccc8)cccc7c56)c4)cc3)c3ccc(-c4c([2H])c([2H])c([2H])c([2H])c4[2H])cc3)cc2)c([2H])c1[2H] ; c1ccc(-c2ccc(N(c3ccccc3)c3ccc(-c4cc(-c5ccc6c(c5)oc5ccccc56)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5ccc6c(c5)oc5ccccc56)cc(-c5ccc(-c6ccccc6)c6oc7c(-c8ccccc8)cccc7c56)c4)cc3)cc2)cc1]

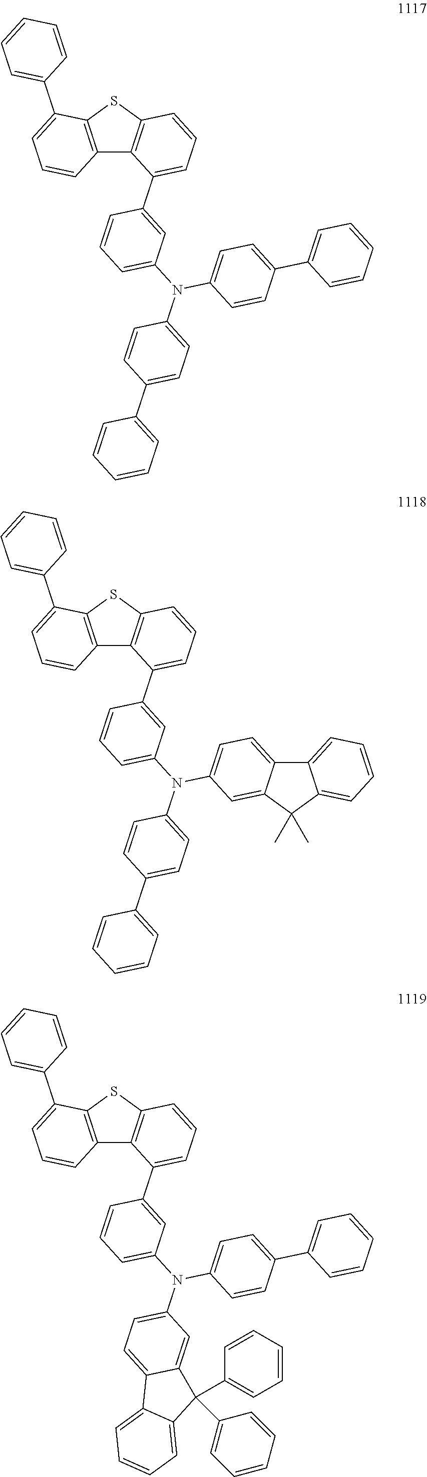 [SMILES: c1ccc(-c2ccc(N(c3ccc(-c4ccccc4)cc3)c3cccc(-c4cccc5sc6c(-c7ccccc7)cccc6c45)c3)cc2)cc1 ; CC1(C)c2ccccc2-c2ccc(N(c3ccc(-c4ccccc4)cc3)c3cccc(-c4cccc5sc6c(-c7ccccc7)cccc6c45)c3)cc21 ; c1ccc(-c2ccc(N(c3cccc(-c4cccc5sc6c(-c7ccccc7)cccc6c45)c3)c3ccc4c(c3)C(c3ccccc3)(c3ccccc3)c3ccccc3-4)cc2)cc1]

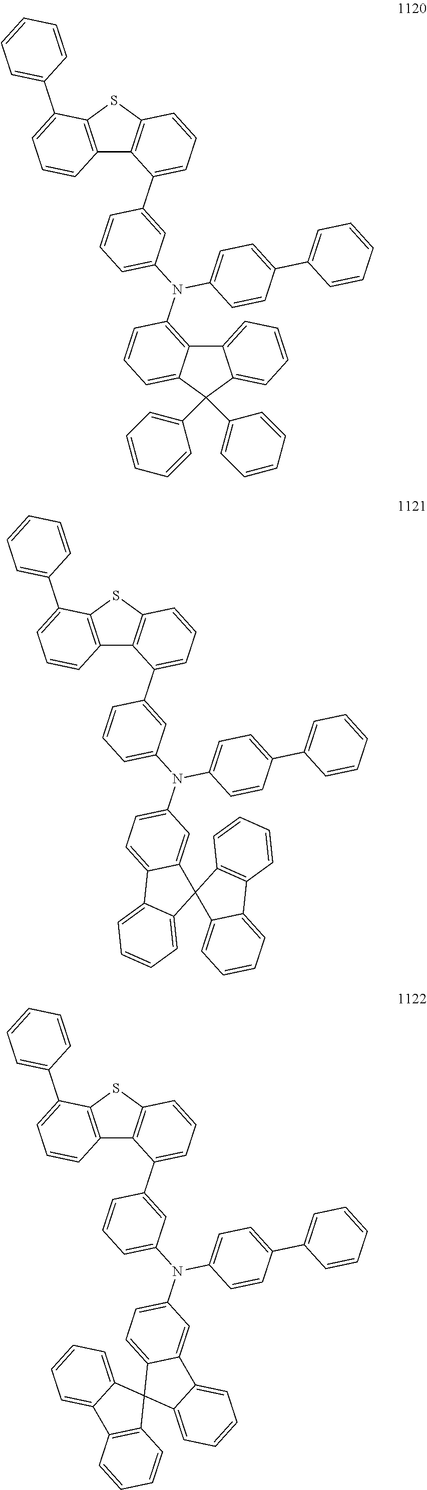 [SMILES: c1ccc(-c2ccc(N(c3cccc(-c4cccc5sc6c(-c7ccccc7)cccc6c45)c3)c3cccc4c3-c3ccccc3C4(c3ccccc3)c3ccccc3)cc2)cc1 ; c1ccc(-c2ccc(N(c3cccc(-c4cccc5sc6c(-c7ccccc7)cccc6c45)c3)c3ccc4c(c3)C3(c5ccccc5-c5ccccc53)c3ccccc3-4)cc2)cc1 ; c1ccc(-c2ccc(N(c3cccc(-c4cccc5sc6c(-c7ccccc7)cccc6c45)c3)c3ccc4c(c3)-c3ccccc3C43c4ccccc4-c4ccccc43)cc2)cc1]

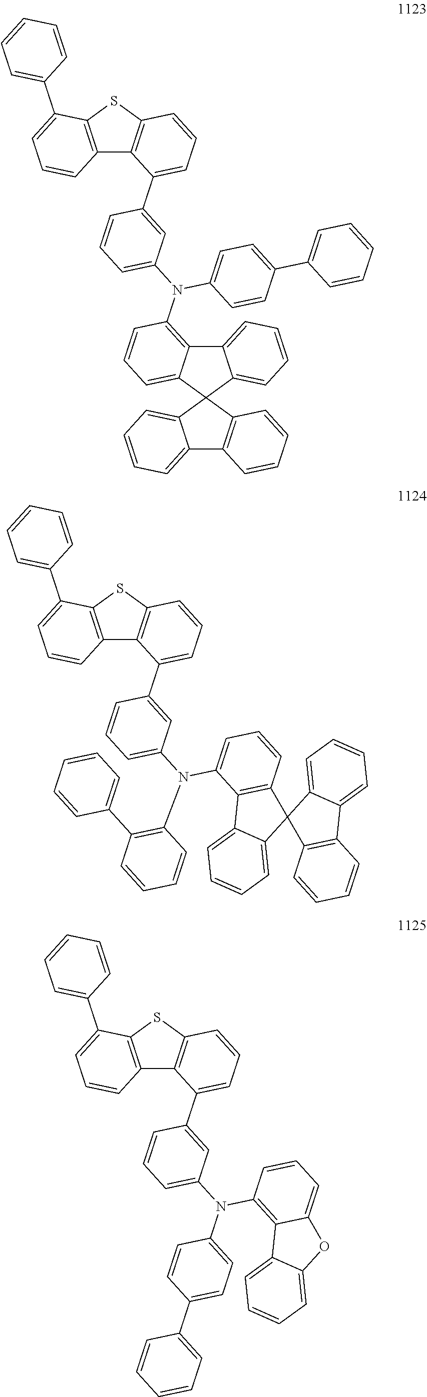 [SMILES: c1ccc(-c2ccc(N(c3cccc(-c4cccc5sc6c(-c7ccccc7)cccc6c45)c3)c3cccc4c3-c3ccccc3C43c4ccccc4-c4ccccc43)cc2)cc1 ; c1ccc(-c2ccccc2N(c2cccc(-c3cccc4sc5c(-c6ccccc6)cccc5c34)c2)c2cccc3c2-c2ccccc2C32c3ccccc3-c3ccccc32)cc1 ; c1ccc(-c2ccc(N(c3cccc(-c4cccc5sc6c(-c7ccccc7)cccc6c45)c3)c3cccc4oc5ccccc5c34)cc2)cc1]

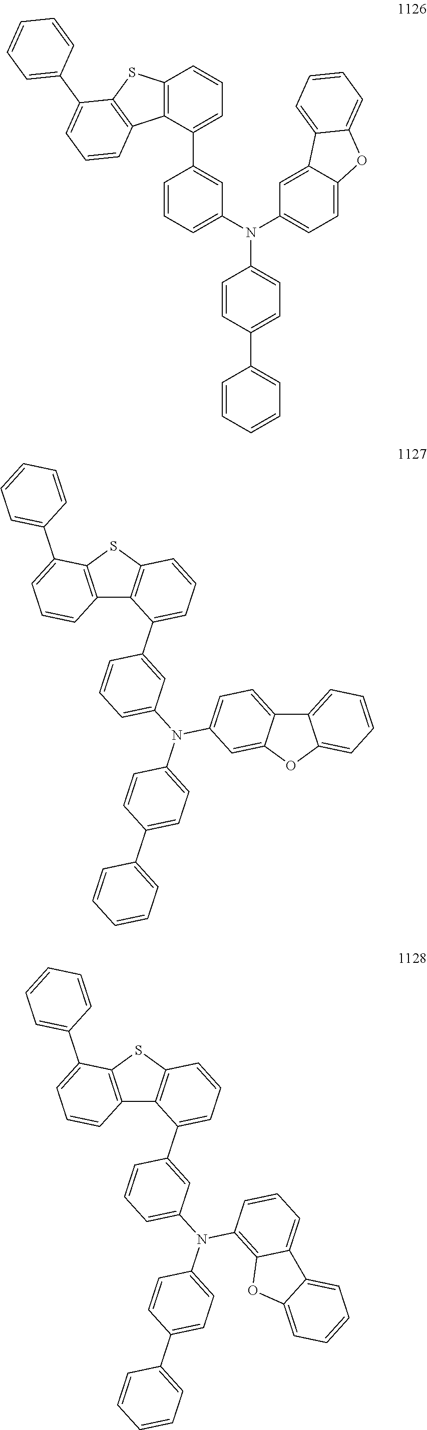 [SMILES: c1ccc(-c2ccc(N(c3cccc(-c4cccc5sc6c(-c7ccccc7)cccc6c45)c3)c3ccc4oc5ccccc5c4c3)cc2)cc1 ; c1ccc(-c2ccc(N(c3cccc(-c4cccc5sc6c(-c7ccccc7)cccc6c45)c3)c3ccc4c(c3)oc3ccccc34)cc2)cc1 ; c1ccc(-c2ccc(N(c3cccc(-c4cccc5sc6c(-c7ccccc7)cccc6c45)c3)c3cccc4c3oc3ccccc34)cc2)cc1]

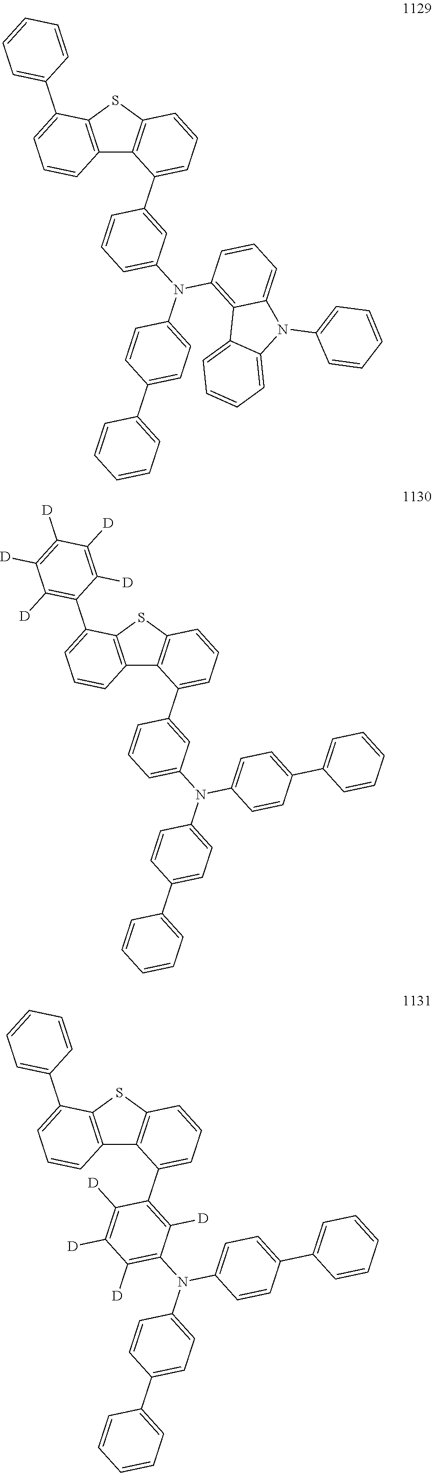

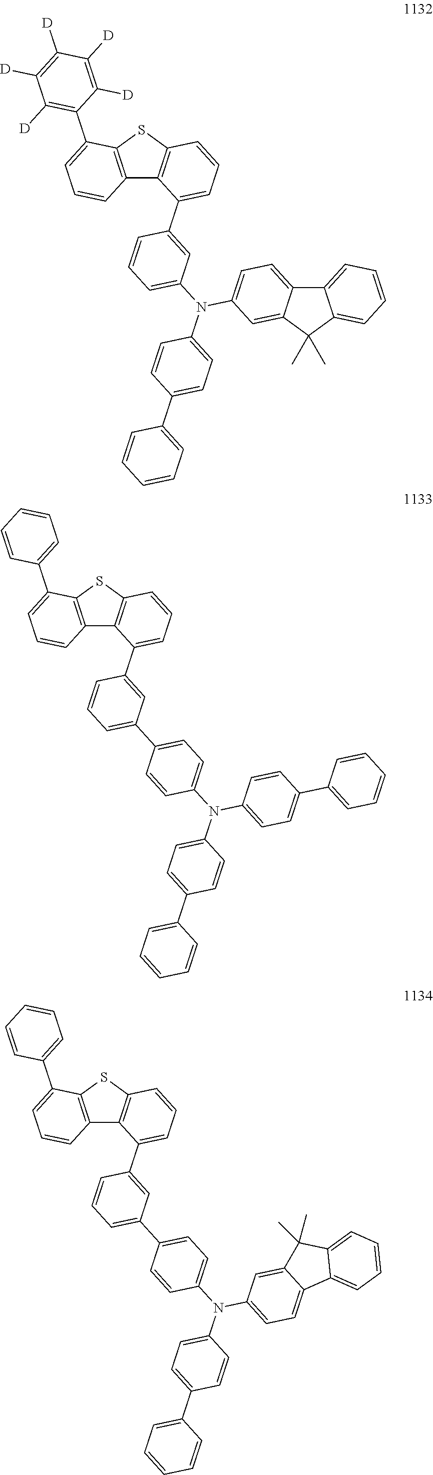 [SMILES: [2H]c1c([2H])c([2H])c(-c2cccc3c2sc2cccc(-c4cccc(N(c5ccc(-c6ccccc6)cc5)c5ccc6c(c5)C(C)(C)c5ccccc5-6)c4)c23)c([2H])c1[2H] ; c1ccc(-c2ccc(N(c3ccc(-c4ccccc4)cc3)c3ccc(-c4cccc(-c5cccc6sc7c(-c8ccccc8)cccc7c56)c4)cc3)cc2)cc1 ; CC1(C)c2ccccc2-c2ccc(N(c3ccc(-c4ccccc4)cc3)c3ccc(-c4cccc(-c5cccc6sc7c(-c8ccccc8)cccc7c56)c4)cc3)cc21]

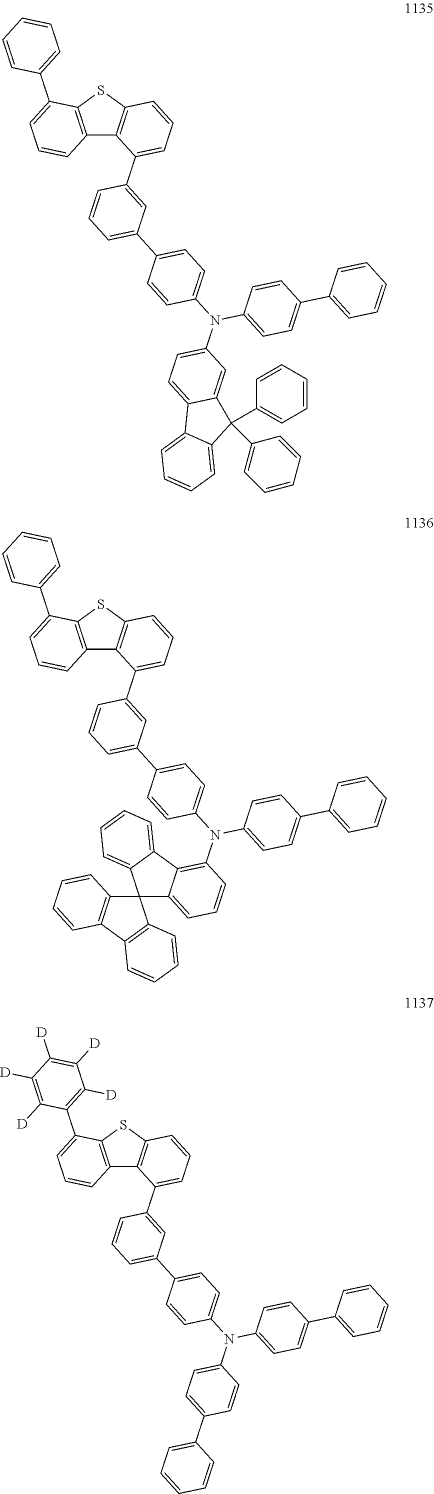 [SMILES: c1ccc(-c2ccc(N(c3ccc(-c4cccc(-c5cccc6sc7c(-c8ccccc8)cccc7c56)c4)cc3)c3ccc4c(c3)C(c3ccccc3)(c3ccccc3)c3ccccc3-4)cc2)cc1 ; c1ccc(-c2ccc(N(c3ccc(-c4cccc(-c5cccc6sc7c(-c8ccccc8)cccc7c56)c4)cc3)c3cccc4c3-c3ccccc3C43c4ccccc4-c4ccccc43)cc2)cc1 ; [2H]c1c([2H])c([2H])c(-c2cccc3c2sc2cccc(-c4cccc(-c5ccc(N(c6ccc(-c7ccccc7)cc6)c6ccc(-c7ccccc7)cc6)cc5)c4)c23)c([2H])c1[2H]]

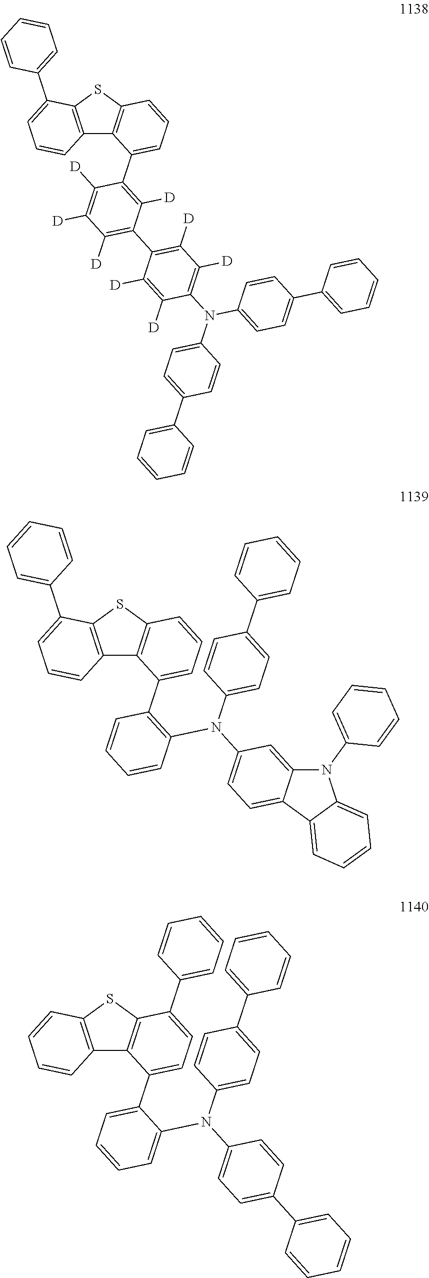 [SMILES: [2H]c1c([2H])c(-c2c([2H])c([2H])c(N(c3ccc(-c4ccccc4)cc3)c3ccc(-c4ccccc4)cc3)c([2H])c2[2H])c([2H])c(-c2cccc3sc4c(-c5ccccc5)cccc4c23)c1[2H] ; c1ccc(-c2ccc(N(c3ccc4c5ccccc5n(-c5ccccc5)c4c3)c3ccccc3-c3cccc4sc5c(-c6ccccc6)cccc5c34)cc2)cc1 ; c1ccc(-c2ccc(N(c3ccc(-c4ccccc4)cc3)c3ccccc3-c3ccc(-c4ccccc4)c4sc5ccccc5c34)cc2)cc1]

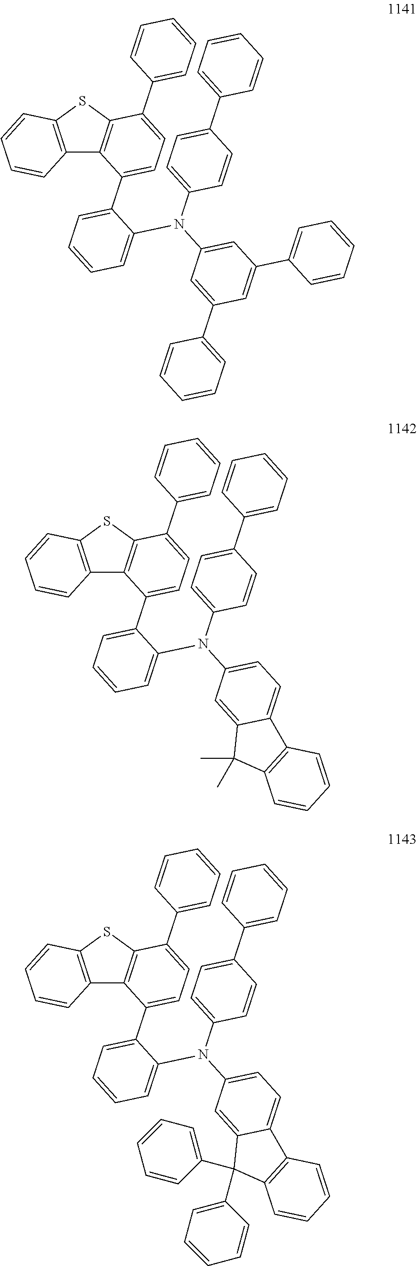

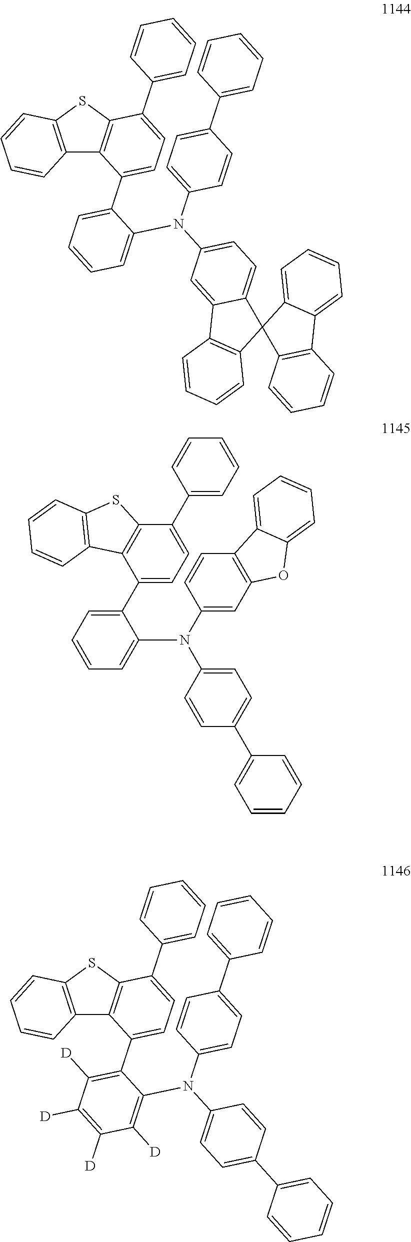

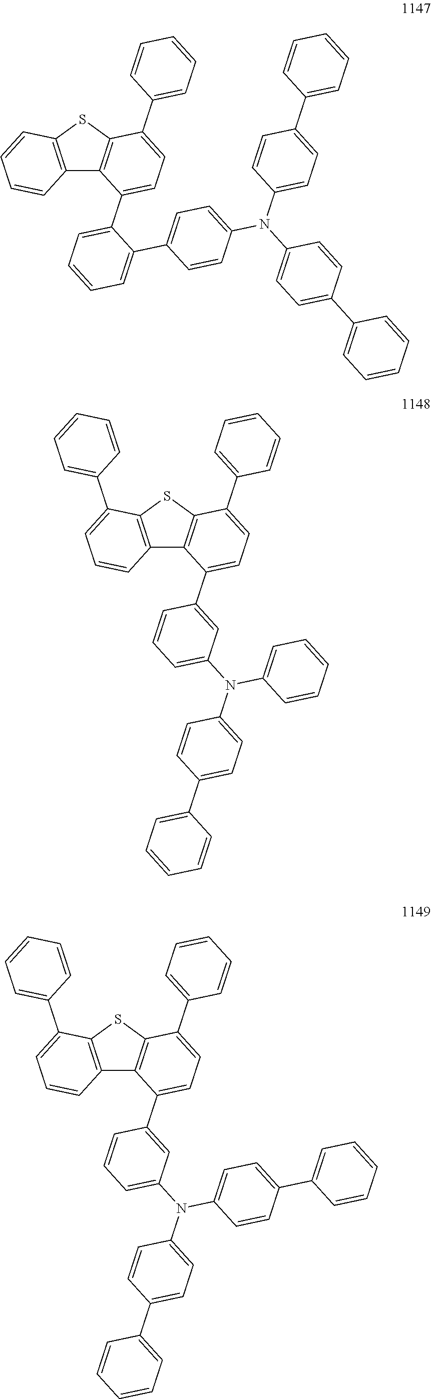 [SMILES: c1ccc(-c2ccc(N(c3ccc(-c4ccccc4)cc3)c3ccc(-c4ccccc4-c4ccc(-c5ccccc5)c5sc6ccccc6c45)cc3)cc2)cc1 ; c1ccc(-c2ccc(N(c3ccccc3)c3cccc(-c4ccc(-c5ccccc5)c5sc6c(-c7ccccc7)cccc6c45)c3)cc2)cc1 ; c1ccc(-c2ccc(N(c3ccc(-c4ccccc4)cc3)c3cccc(-c4ccc(-c5ccccc5)c5sc6c(-c7ccccc7)cccc6c45)c3)cc2)cc1]

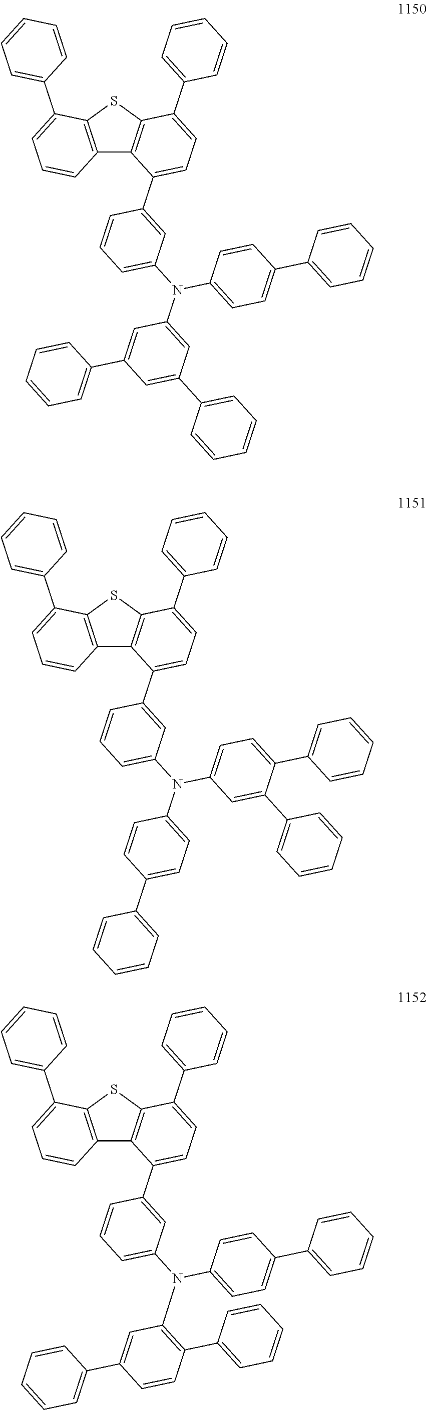 [SMILES: c1ccc(-c2ccc(N(c3cc(-c4ccccc4)cc(-c4ccccc4)c3)c3cccc(-c4ccc(-c5ccccc5)c5sc6c(-c7ccccc7)cccc6c45)c3)cc2)cc1 ; c1ccc(-c2ccc(N(c3cccc(-c4ccc(-c5ccccc5)c5sc6c(-c7ccccc7)cccc6c45)c3)c3ccc(-c4ccccc4)c(-c4ccccc4)c3)cc2)cc1 ; c1ccc(-c2ccc(N(c3cccc(-c4ccc(-c5ccccc5)c5sc6c(-c7ccccc7)cccc6c45)c3)c3cc(-c4ccccc4)ccc3-c3ccccc3)cc2)cc1]

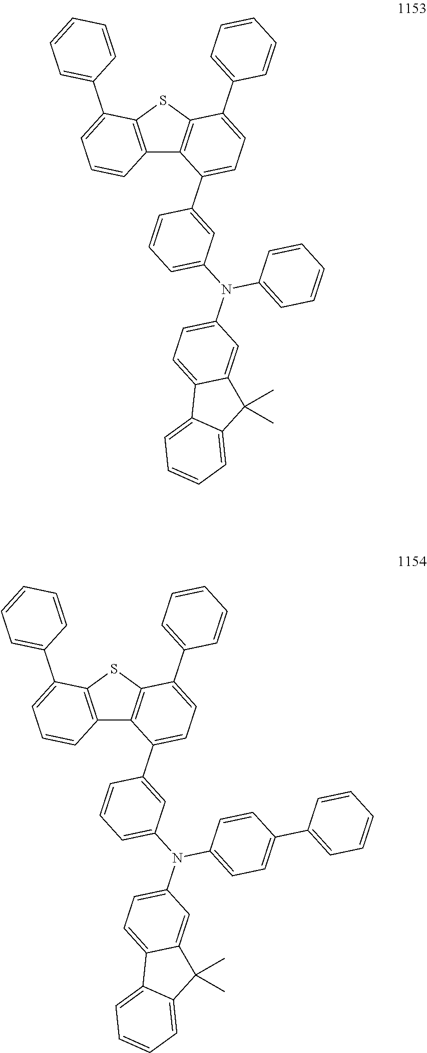 [SMILES: CC1(C)c2ccccc2-c2ccc(N(c3ccccc3)c3cccc(-c4ccc(-c5ccccc5)c5sc6c(-c7ccccc7)cccc6c45)c3)cc21 ; CC1(C)c2ccccc2-c2ccc(N(c3ccc(-c4ccccc4)cc3)c3cccc(-c4ccc(-c5ccccc5)c5sc6c(-c7ccccc7)cccc6c45)c3)cc21]

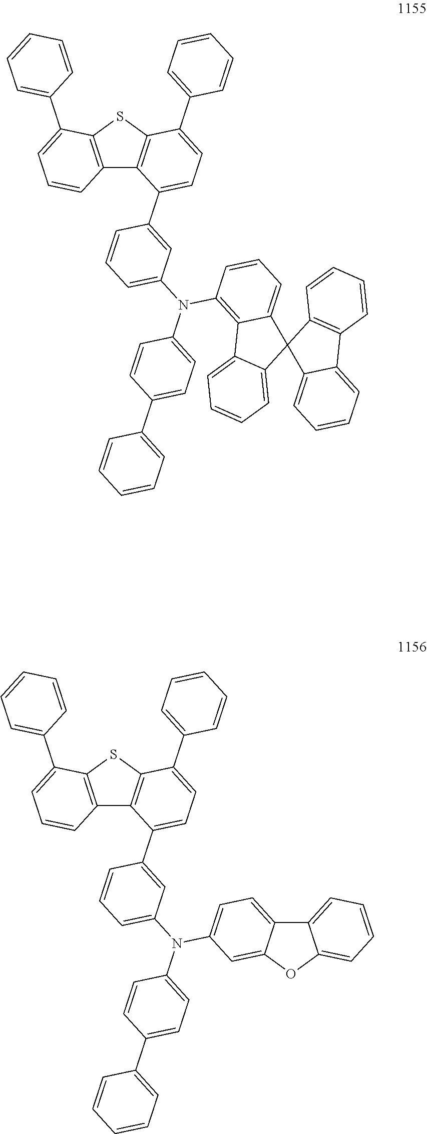 [SMILES: c1ccc(-c2ccc(N(c3cccc(-c4ccc(-c5ccccc5)c5sc6c(-c7ccccc7)cccc6c45)c3)c3cccc4c3-c3ccccc3C43c4ccccc4-c4ccccc43)cc2)cc1 ; c1ccc(-c2ccc(N(c3cccc(-c4ccc(-c5ccccc5)c5sc6c(-c7ccccc7)cccc6c45)c3)c3ccc4c(c3)oc3ccccc34)cc2)cc1]

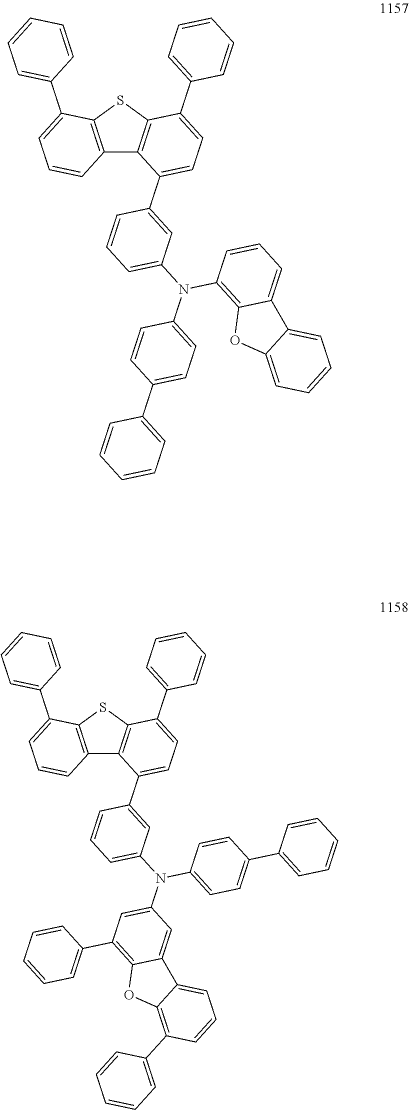 [SMILES: c1ccc(-c2ccc(N(c3cccc(-c4ccc(-c5ccccc5)c5sc6c(-c7ccccc7)cccc6c45)c3)c3cccc4c3oc3ccccc34)cc2)cc1 ; c1ccc(-c2ccc(N(c3cccc(-c4ccc(-c5ccccc5)c5sc6c(-c7ccccc7)cccc6c45)c3)c3cc(-c4ccccc4)c4oc5c(-c6ccccc6)cccc5c4c3)cc2)cc1]

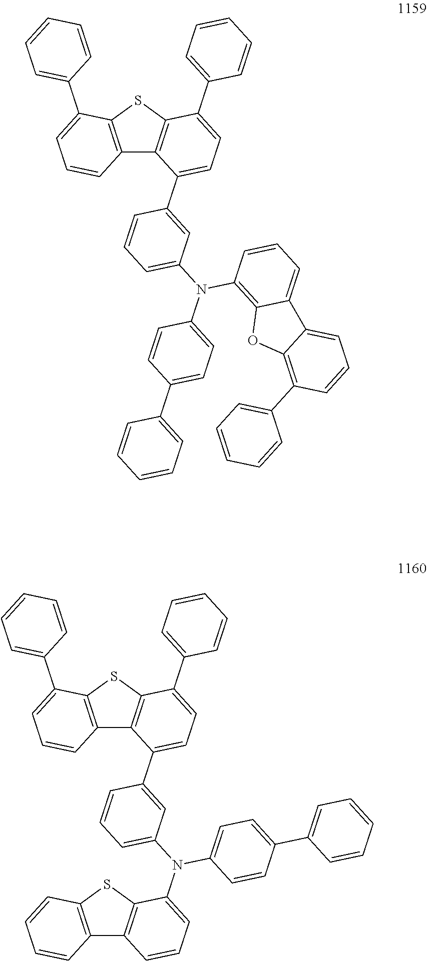 [SMILES: c1ccc(-c2ccc(N(c3cccc(-c4ccc(-c5ccccc5)c5sc6c(-c7ccccc7)cccc6c45)c3)c3cccc4c3oc3c(-c5ccccc5)cccc34)cc2)cc1 ; c1ccc(-c2ccc(N(c3cccc(-c4ccc(-c5ccccc5)c5sc6c(-c7ccccc7)cccc6c45)c3)c3cccc4c3sc3ccccc34)cc2)cc1]

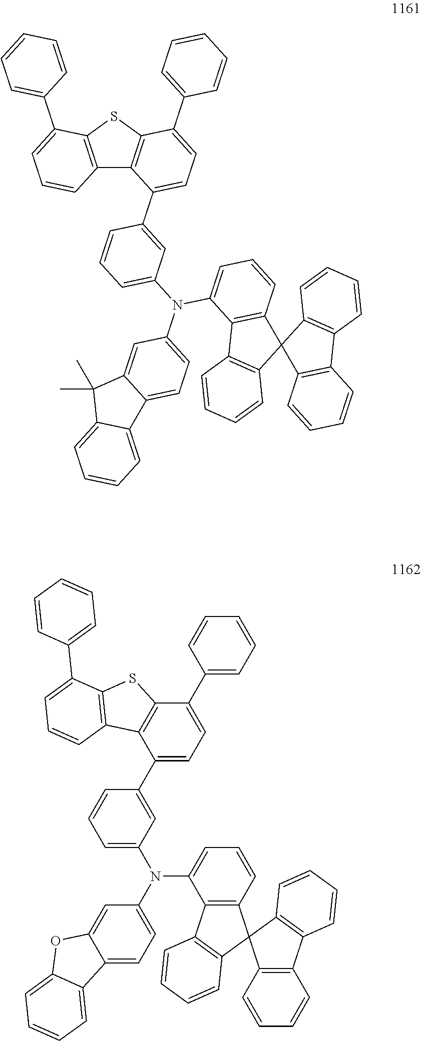 [SMILES: CC1(C)c2ccccc2-c2ccc(N(c3cccc(-c4ccc(-c5ccccc5)c5sc6c(-c7ccccc7)cccc6c45)c3)c3cccc4c3-c3ccccc3C43c4ccccc4-c4ccccc43)cc21 ; c1ccc(-c2cccc3c2sc2c(-c4ccccc4)ccc(-c4cccc(N(c5ccc6c(c5)oc5ccccc56)c5cccc6c5-c5ccccc5C65c6ccccc6-c6ccccc65)c4)c23)cc1]

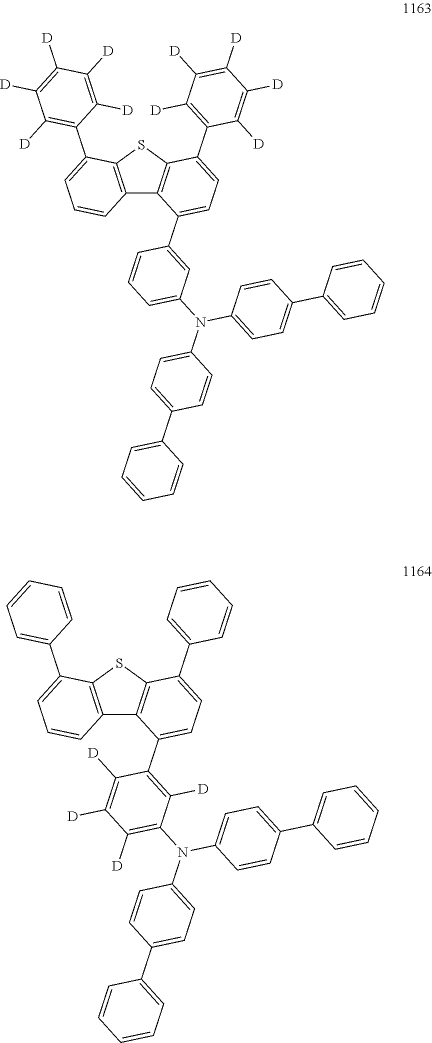 [SMILES: [2H]c1c([2H])c([2H])c(-c2cccc3c2sc2c(-c4c([2H])c([2H])c([2H])c([2H])c4[2H])ccc(-c4cccc(N(c5ccc(-c6ccccc6)cc5)c5ccc(-c6ccccc6)cc5)c4)c23)c([2H])c1[2H] ; [2H]c1c([2H])c(-c2ccc(-c3ccccc3)c3sc4c(-c5ccccc5)cccc4c23)c([2H])c(N(c2ccc(-c3ccccc3)cc2)c2ccc(-c3ccccc3)cc2)c1[2H]]

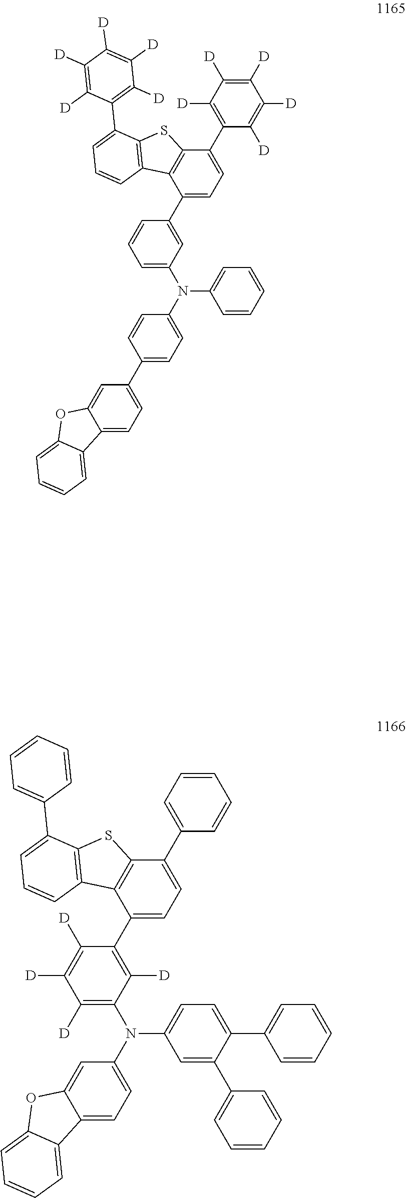 [SMILES: [2H]c1c([2H])c([2H])c(-c2cccc3c2sc2c(-c4c([2H])c([2H])c([2H])c([2H])c4[2H])ccc(-c4cccc(N(c5ccccc5)c5ccc(-c6ccc7c(c6)oc6ccccc67)cc5)c4)c23)c([2H])c1[2H] ; [2H]c1c([2H])c(-c2ccc(-c3ccccc3)c3sc4c(-c5ccccc5)cccc4c23)c([2H])c(N(c2ccc(-c3ccccc3)c(-c3ccccc3)c2)c2ccc3c(c2)oc2ccccc23)c1[2H]]

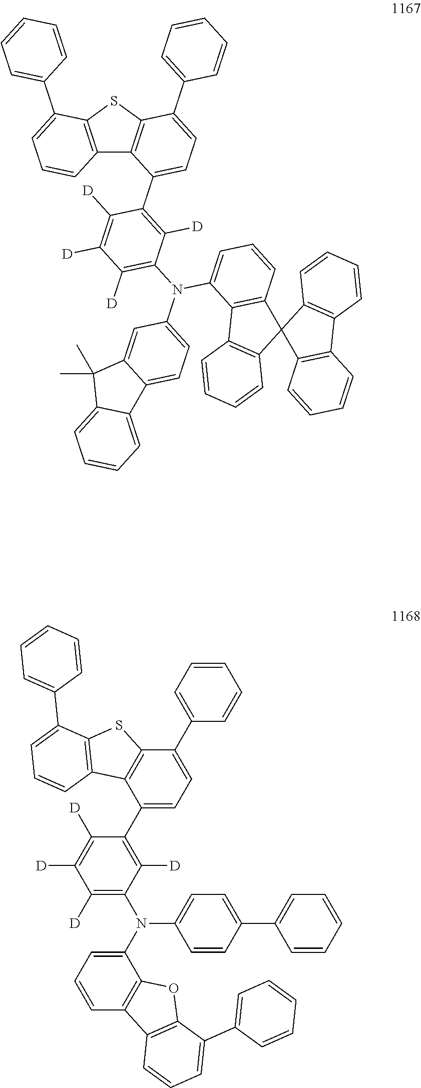 [SMILES: [2H]c1c([2H])c(-c2ccc(-c3ccccc3)c3sc4c(-c5ccccc5)cccc4c23)c([2H])c(N(c2ccc3c(c2)C(C)(C)c2ccccc2-3)c2cccc3c2-c2ccccc2C32c3ccccc3-c3ccccc32)c1[2H] ; [2H]c1c([2H])c(-c2ccc(-c3ccccc3)c3sc4c(-c5ccccc5)cccc4c23)c([2H])c(N(c2ccc(-c3ccccc3)cc2)c2cccc3c2oc2c(-c4ccccc4)cccc23)c1[2H]]

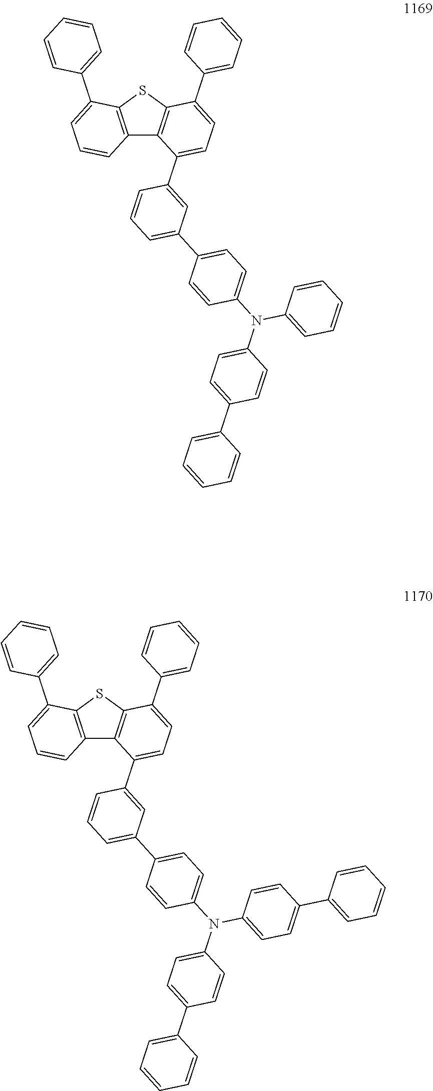 [SMILES: c1ccc(-c2ccc(N(c3ccccc3)c3ccc(-c4cccc(-c5ccc(-c6ccccc6)c6sc7c(-c8ccccc8)cccc7c56)c4)cc3)cc2)cc1 ; c1ccc(-c2ccc(N(c3ccc(-c4ccccc4)cc3)c3ccc(-c4cccc(-c5ccc(-c6ccccc6)c6sc7c(-c8ccccc8)cccc7c56)c4)cc3)cc2)cc1]

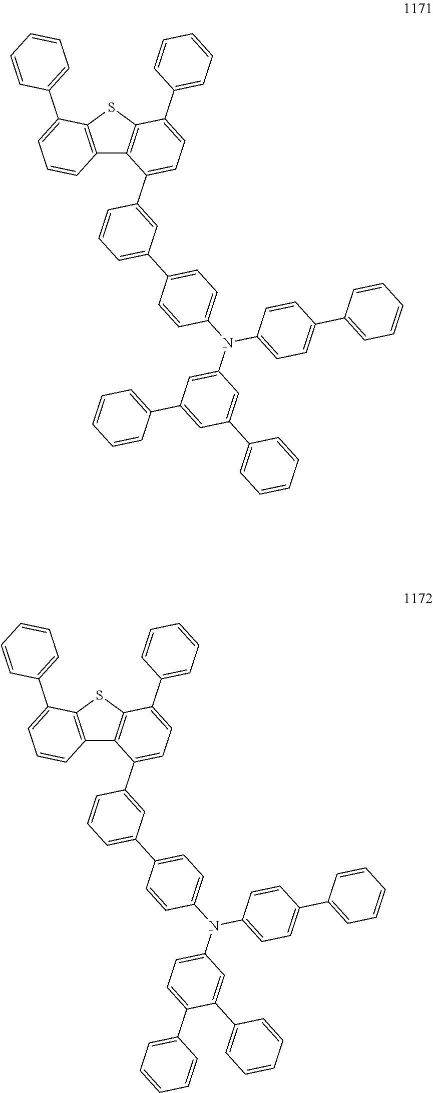 [SMILES: c1ccc(-c2ccc(N(c3ccc(-c4cccc(-c5ccc(-c6ccccc6)c6sc7c(-c8ccccc8)cccc7c56)c4)cc3)c3cc(-c4ccccc4)cc(-c4ccccc4)c3)cc2)cc1 ; c1ccc(-c2ccc(N(c3ccc(-c4cccc(-c5ccc(-c6ccccc6)c6sc7c(-c8ccccc8)cccc7c56)c4)cc3)c3ccc(-c4ccccc4)c(-c4ccccc4)c3)cc2)cc1]

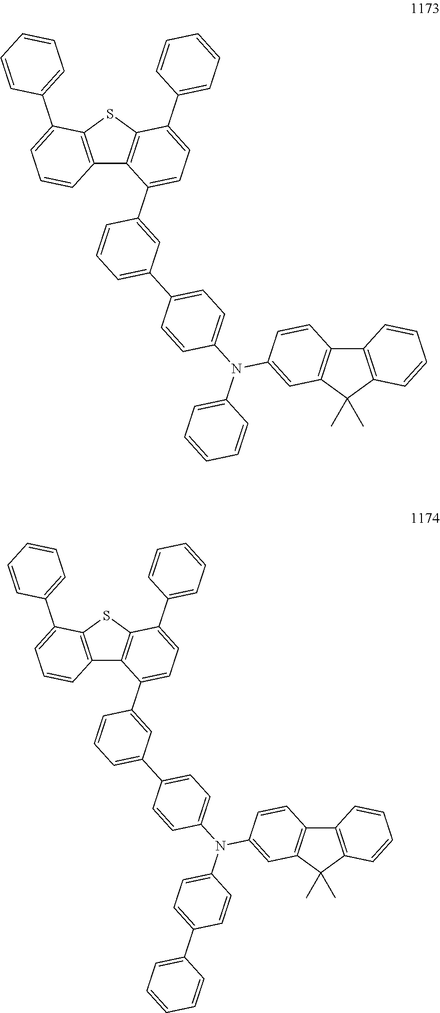 [SMILES: CC1(C)c2ccccc2-c2ccc(N(c3ccccc3)c3ccc(-c4cccc(-c5ccc(-c6ccccc6)c6sc7c(-c8ccccc8)cccc7c56)c4)cc3)cc21 ; CC1(C)c2ccccc2-c2ccc(N(c3ccc(-c4ccccc4)cc3)c3ccc(-c4cccc(-c5ccc(-c6ccccc6)c6sc7c(-c8ccccc8)cccc7c56)c4)cc3)cc21]

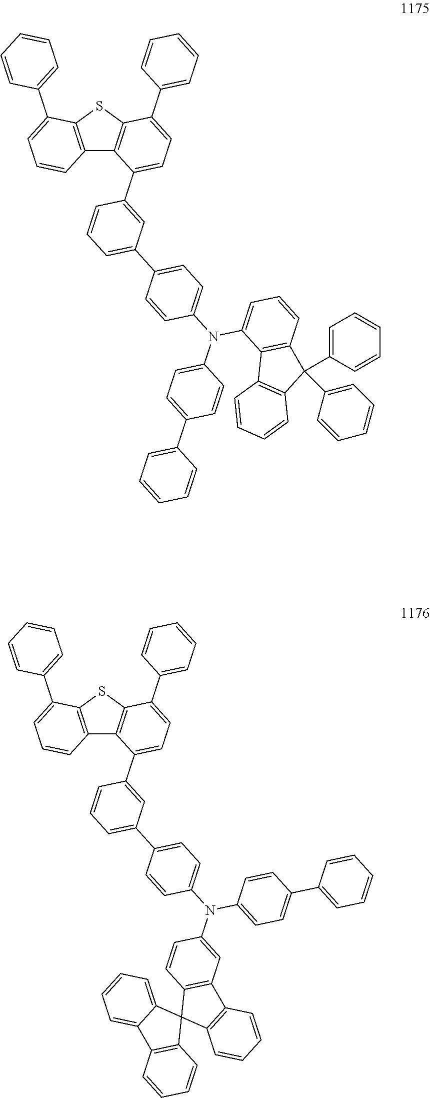 [SMILES: c1ccc(-c2ccc(N(c3ccc(-c4cccc(-c5ccc(-c6ccccc6)c6sc7c(-c8ccccc8)cccc7c56)c4)cc3)c3cccc4c3-c3ccccc3C4(c3ccccc3)c3ccccc3)cc2)cc1 ; c1ccc(-c2ccc(N(c3ccc(-c4cccc(-c5ccc(-c6ccccc6)c6sc7c(-c8ccccc8)cccc7c56)c4)cc3)c3ccc4c(c3)-c3ccccc3C43c4ccccc4-c4ccccc43)cc2)cc1]

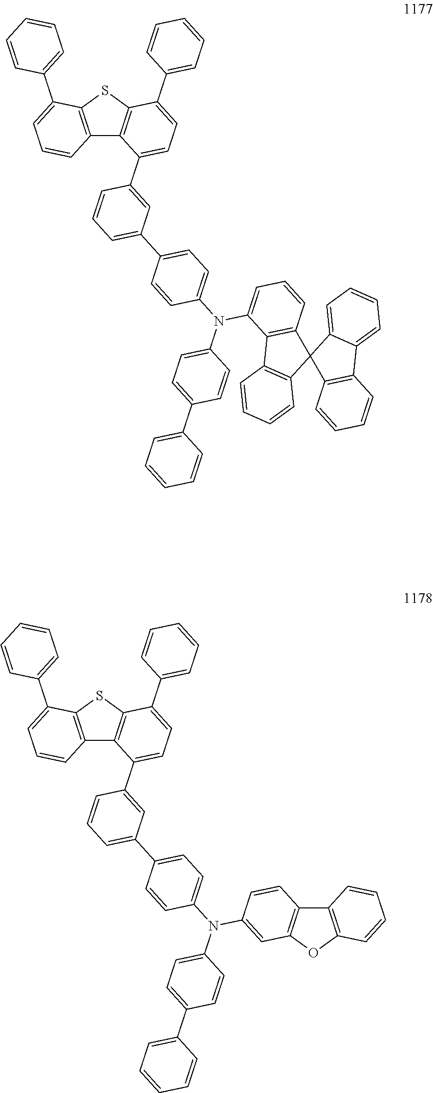 [SMILES: c1ccc(-c2ccc(N(c3ccc(-c4cccc(-c5ccc(-c6ccccc6)c6sc7c(-c8ccccc8)cccc7c56)c4)cc3)c3cccc4c3-c3ccccc3C43c4ccccc4-c4ccccc43)cc2)cc1 ; c1ccc(-c2ccc(N(c3ccc(-c4cccc(-c5ccc(-c6ccccc6)c6sc7c(-c8ccccc8)cccc7c56)c4)cc3)c3ccc4c(c3)oc3ccccc34)cc2)cc1]

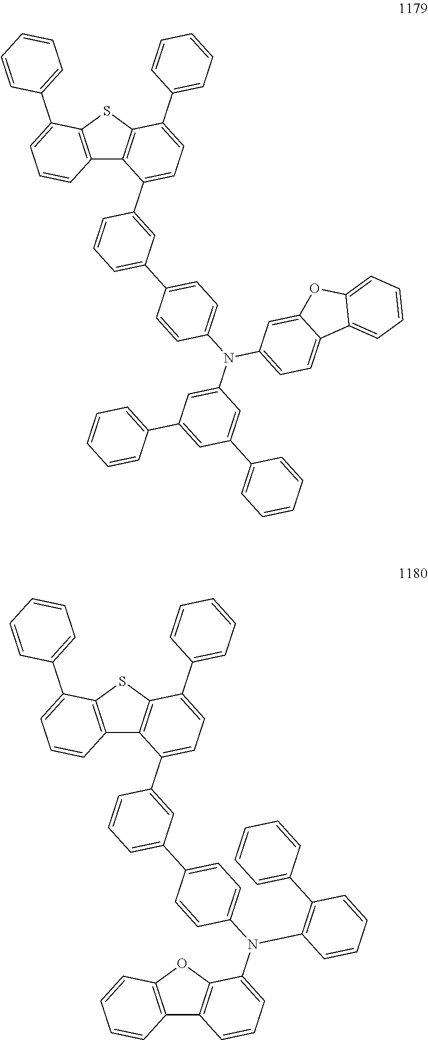 [SMILES: c1ccc(-c2cc(-c3ccccc3)cc(N(c3ccc(-c4cccc(-c5ccc(-c6ccccc6)c6sc7c(-c8ccccc8)cccc7c56)c4)cc3)c3ccc4c(c3)oc3ccccc34)c2)cc1 ; c1ccc(-c2ccccc2N(c2ccc(-c3cccc(-c4ccc(-c5ccccc5)c5sc6c(-c7ccccc7)cccc6c45)c3)cc2)c2cccc3c2oc2ccccc23)cc1]

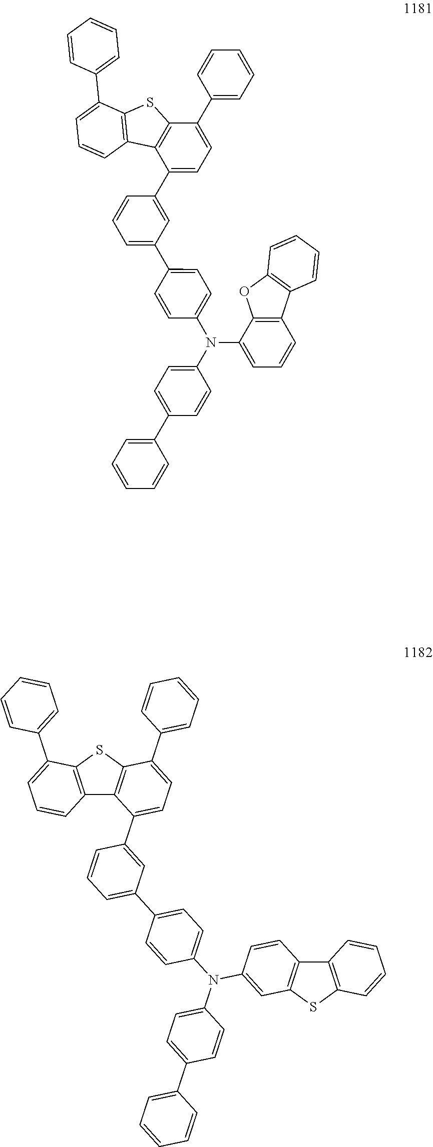 [SMILES: c1ccc(-c2ccc(N(c3ccc(-c4cccc(-c5ccc(-c6ccccc6)c6sc7c(-c8ccccc8)cccc7c56)c4)cc3)c3cccc4c3oc3ccccc34)cc2)cc1 ; c1ccc(-c2ccc(N(c3ccc(-c4cccc(-c5ccc(-c6ccccc6)c6sc7c(-c8ccccc8)cccc7c56)c4)cc3)c3ccc4c(c3)sc3ccccc34)cc2)cc1]

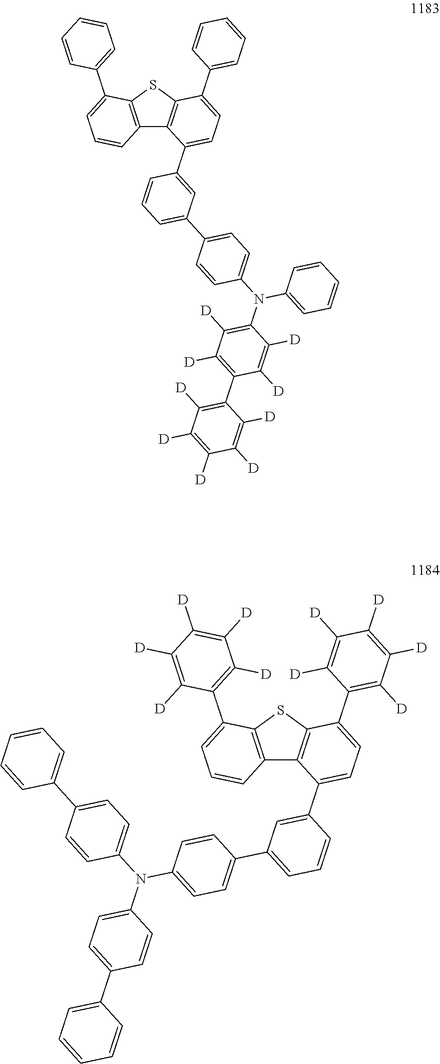 [SMILES: [2H]c1c([2H])c([2H])c(-c2c([2H])c([2H])c(N(c3ccccc3)c3ccc(-c4cccc(-c5ccc(-c6ccccc6)c6sc7c(-c8ccccc8)cccc7c56)c4)cc3)c([2H])c2[2H])c([2H])c1[2H] ; [2H]c1c([2H])c([2H])c(-c2cccc3c2sc2c(-c4c([2H])c([2H])c([2H])c([2H])c4[2H])ccc(-c4cccc(-c5ccc(N(c6ccc(-c7ccccc7)cc6)c6ccc(-c7ccccc7)cc6)cc5)c4)c23)c([2H])c1[2H]]

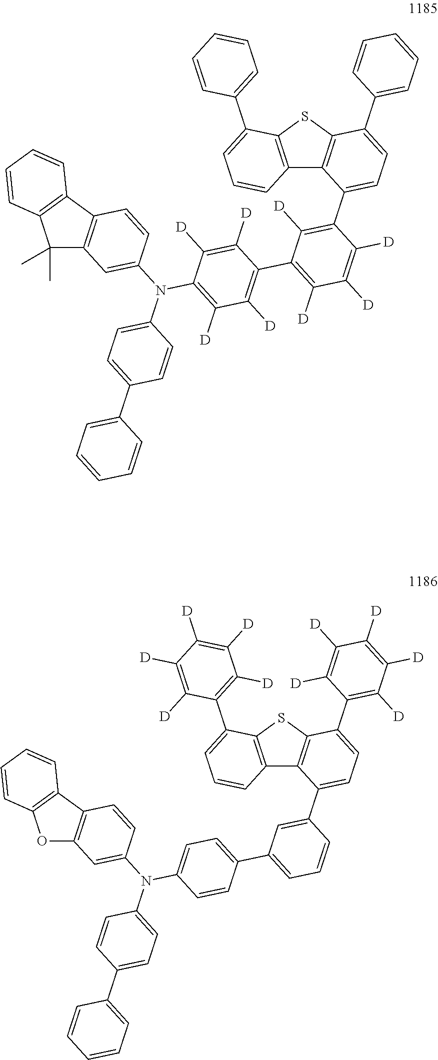 [SMILES: [2H]c1c([2H])c(-c2c([2H])c([2H])c(N(c3ccc(-c4ccccc4)cc3)c3ccc4c(c3)C(C)(C)c3ccccc3-4)c([2H])c2[2H])c([2H])c(-c2ccc(-c3ccccc3)c3sc4c(-c5ccccc5)cccc4c23)c1[2H] ; [2H]c1c([2H])c([2H])c(-c2cccc3c2sc2c(-c4c([2H])c([2H])c([2H])c([2H])c4[2H])ccc(-c4cccc(-c5ccc(N(c6ccc(-c7ccccc7)cc6)c6ccc7c(c6)oc6ccccc67)cc5)c4)c23)c([2H])c1[2H]]

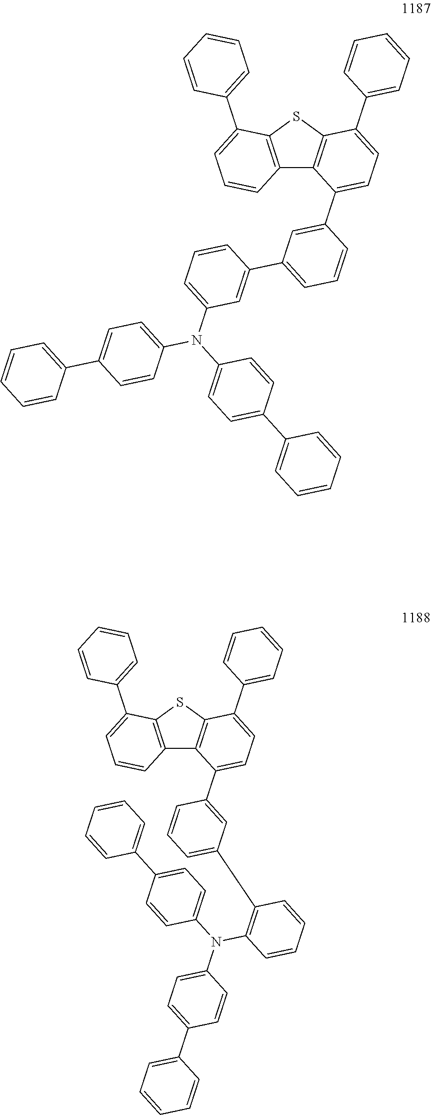 [SMILES: c1ccc(-c2ccc(N(c3ccc(-c4ccccc4)cc3)c3cccc(-c4cccc(-c5ccc(-c6ccccc6)c6sc7c(-c8ccccc8)cccc7c56)c4)c3)cc2)cc1 ; c1ccc(-c2ccc(N(c3ccc(-c4ccccc4)cc3)c3ccccc3-c3cccc(-c4ccc(-c5ccccc5)c5sc6c(-c7ccccc7)cccc6c45)c3)cc2)cc1]

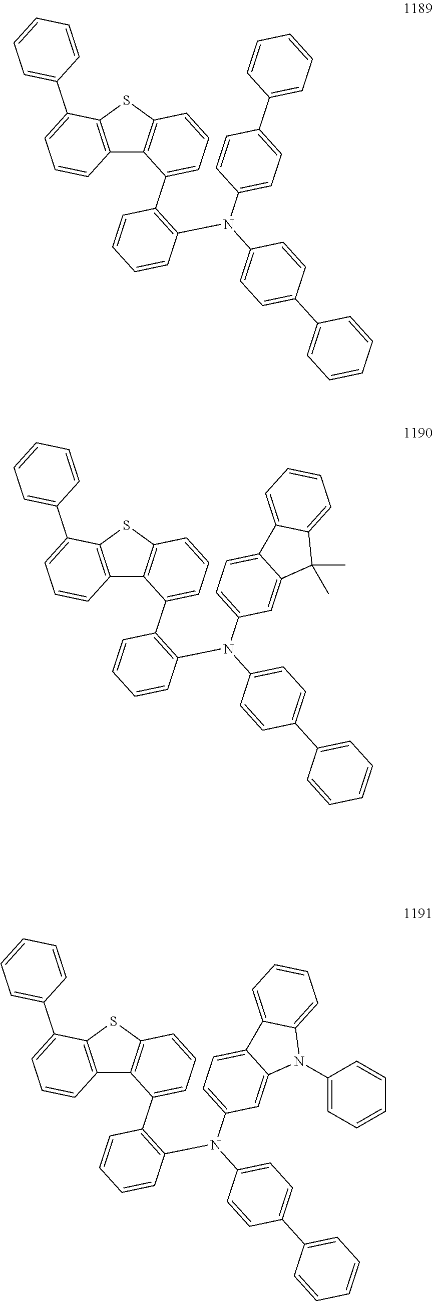 [SMILES: c1ccc(-c2ccc(N(c3ccc(-c4ccccc4)cc3)c3ccccc3-c3cccc4sc5c(-c6ccccc6)cccc5c34)cc2)cc1 ; CC1(C)c2ccccc2-c2ccc(N(c3ccc(-c4ccccc4)cc3)c3ccccc3-c3cccc4sc5c(-c6ccccc6)cccc5c34)cc21 ; c1ccc(-c2ccc(N(c3ccc4c5ccccc5n(-c5ccccc5)c4c3)c3ccccc3-c3cccc4sc5c(-c6ccccc6)cccc5c34)cc2)cc1]

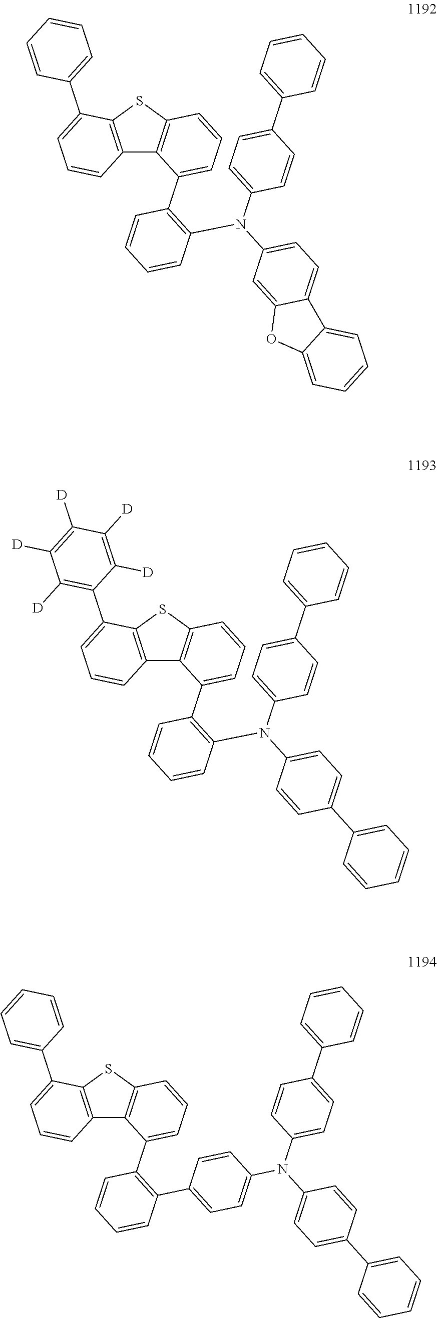 [SMILES: c1ccc(-c2ccc(N(c3ccc4c(c3)oc3ccccc34)c3ccccc3-c3cccc4sc5c(-c6ccccc6)cccc5c34)cc2)cc1 ; [2H]c1c([2H])c([2H])c(-c2cccc3c2sc2cccc(-c4ccccc4N(c4ccc(-c5ccccc5)cc4)c4ccc(-c5ccccc5)cc4)c23)c([2H])c1[2H] ; c1ccc(-c2ccc(N(c3ccc(-c4ccccc4)cc3)c3ccc(-c4ccccc4-c4cccc5sc6c(-c7ccccc7)cccc6c45)cc3)cc2)cc1]

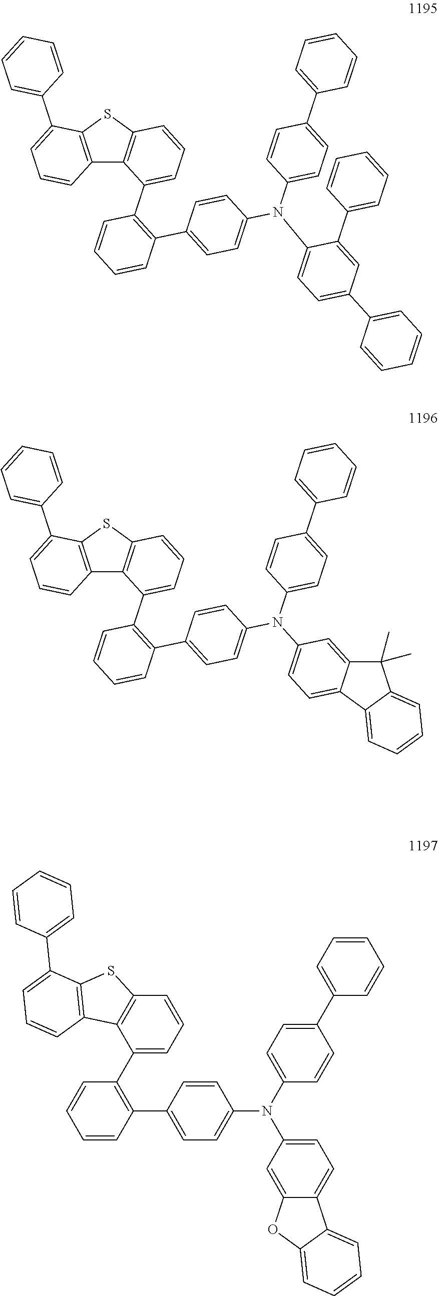 [SMILES: c1ccc(-c2ccc(N(c3ccc(-c4ccccc4-c4cccc5sc6c(-c7ccccc7)cccc6c45)cc3)c3ccc(-c4ccccc4)cc3-c3ccccc3)cc2)cc1 ; CC1(C)c2ccccc2-c2ccc(N(c3ccc(-c4ccccc4)cc3)c3ccc(-c4ccccc4-c4cccc5sc6c(-c7ccccc7)cccc6c45)cc3)cc21 ; c1ccc(-c2ccc(N(c3ccc(-c4ccccc4-c4cccc5sc6c(-c7ccccc7)cccc6c45)cc3)c3ccc4c(c3)oc3ccccc34)cc2)cc1]

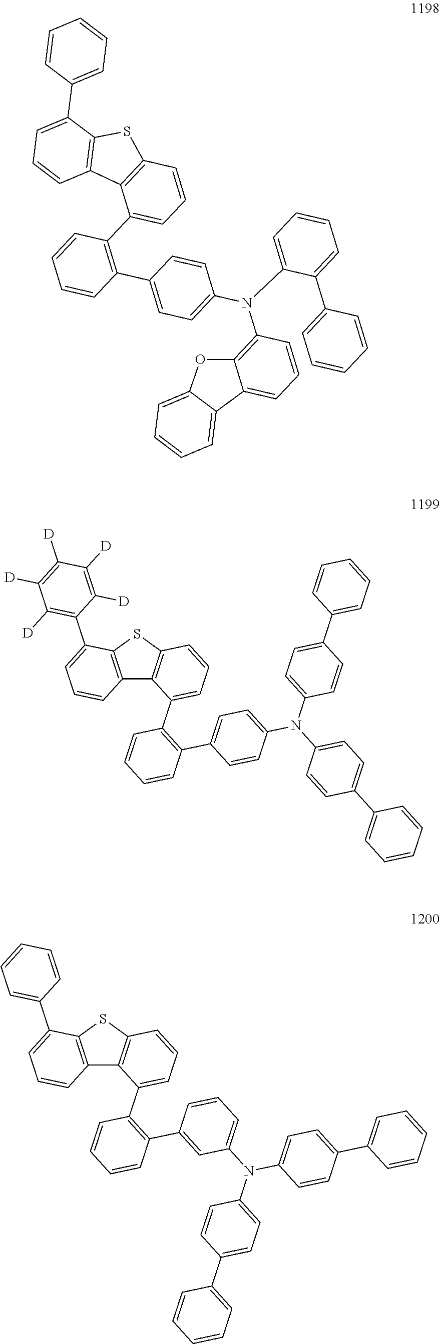 [SMILES: c1ccc(-c2ccccc2N(c2ccc(-c3ccccc3-c3cccc4sc5c(-c6ccccc6)cccc5c34)cc2)c2cccc3c2oc2ccccc23)cc1 ; [2H]c1c([2H])c([2H])c(-c2cccc3c2sc2cccc(-c4ccccc4-c4ccc(N(c5ccc(-c6ccccc6)cc5)c5ccc(-c6ccccc6)cc5)cc4)c23)c([2H])c1[2H] ; c1ccc(-c2ccc(N(c3ccc(-c4ccccc4)cc3)c3cccc(-c4ccccc4-c4cccc5sc6c(-c7ccccc7)cccc6c45)c3)cc2)cc1]

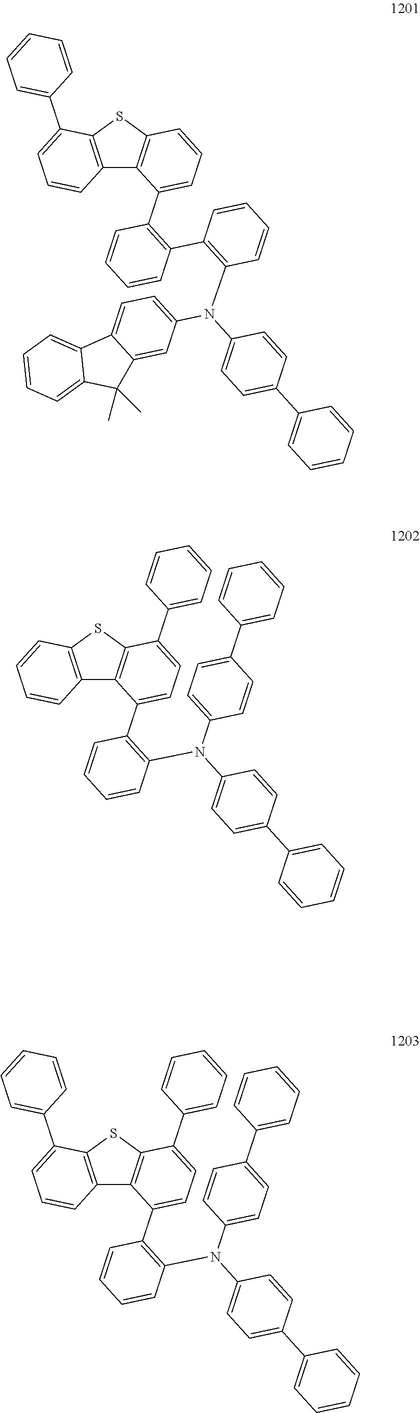 [SMILES: CC1(C)c2ccccc2-c2ccc(N(c3ccc(-c4ccccc4)cc3)c3ccccc3-c3ccccc3-c3cccc4sc5c(-c6ccccc6)cccc5c34)cc21 ; c1ccc(-c2ccc(N(c3ccc(-c4ccccc4)cc3)c3ccccc3-c3ccc(-c4ccccc4)c4sc5ccccc5c34)cc2)cc1 ; c1ccc(-c2ccc(N(c3ccc(-c4ccccc4)cc3)c3ccccc3-c3ccc(-c4ccccc4)c4sc5c(-c6ccccc6)cccc5c34)cc2)cc1]

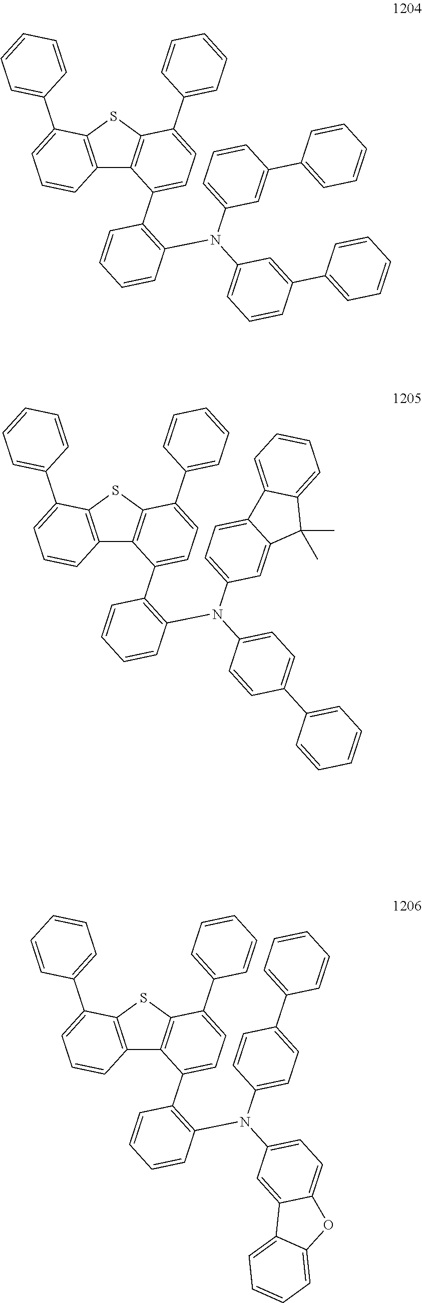 [SMILES: c1ccc(-c2cccc(N(c3cccc(-c4ccccc4)c3)c3ccccc3-c3ccc(-c4ccccc4)c4sc5c(-c6ccccc6)cccc5c34)c2)cc1 ; CC1(C)c2ccccc2-c2ccc(N(c3ccc(-c4ccccc4)cc3)c3ccccc3-c3ccc(-c4ccccc4)c4sc5c(-c6ccccc6)cccc5c34)cc21 ; c1ccc(-c2ccc(N(c3ccc4oc5ccccc5c4c3)c3ccccc3-c3ccc(-c4ccccc4)c4sc5c(-c6ccccc6)cccc5c34)cc2)cc1]

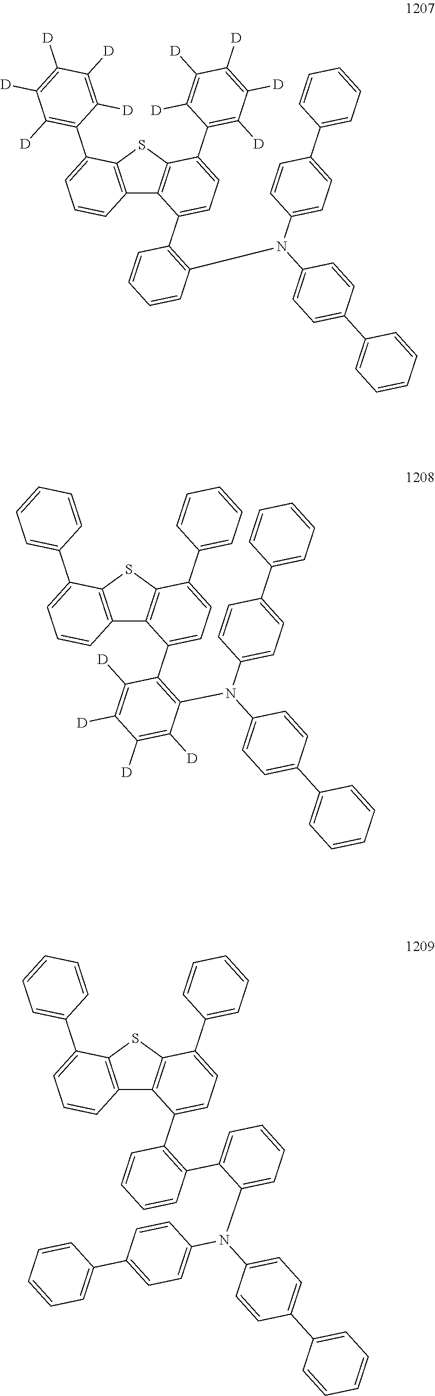 [SMILES: [2H]c1c([2H])c([2H])c(-c2cccc3c2sc2c(-c4c([2H])c([2H])c([2H])c([2H])c4[2H])ccc(-c4ccccc4N(c4ccc(-c5ccccc5)cc4)c4ccc(-c5ccccc5)cc4)c23)c([2H])c1[2H] ; [2H]c1c([2H])c([2H])c(N(c2ccc(-c3ccccc3)cc2)c2ccc(-c3ccccc3)cc2)c(-c2ccc(-c3ccccc3)c3sc4c(-c5ccccc5)cccc4c23)c1[2H] ; c1ccc(-c2ccc(N(c3ccc(-c4ccccc4)cc3)c3ccccc3-c3ccccc3-c3ccc(-c4ccccc4)c4sc5c(-c6ccccc6)cccc5c34)cc2)cc1]

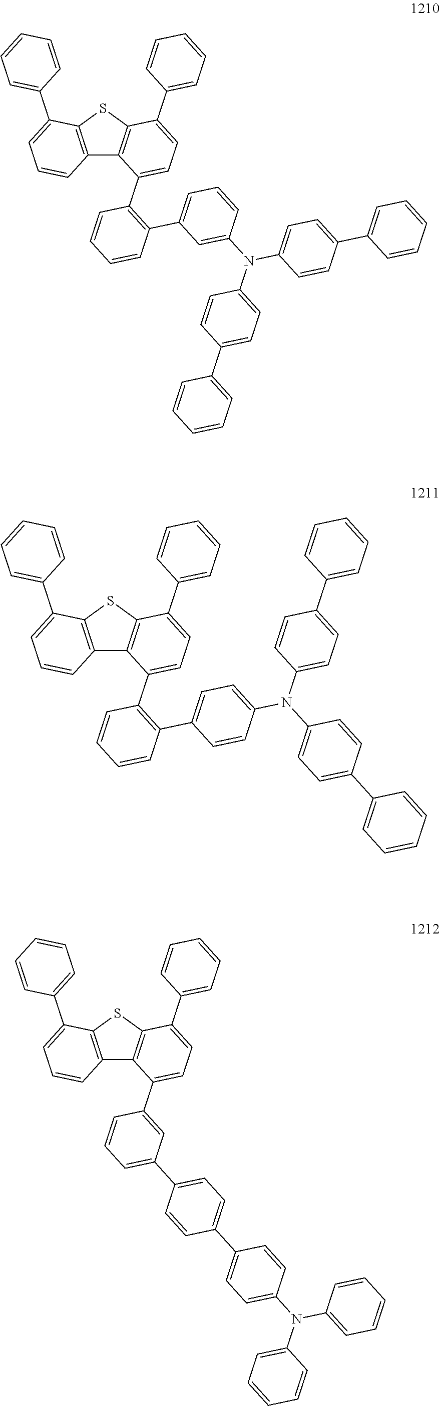 [SMILES: c1ccc(-c2ccc(N(c3ccc(-c4ccccc4)cc3)c3cccc(-c4ccccc4-c4ccc(-c5ccccc5)c5sc6c(-c7ccccc7)cccc6c45)c3)cc2)cc1 ; c1ccc(-c2ccc(N(c3ccc(-c4ccccc4)cc3)c3ccc(-c4ccccc4-c4ccc(-c5ccccc5)c5sc6c(-c7ccccc7)cccc6c45)cc3)cc2)cc1 ; c1ccc(-c2cccc3c2sc2c(-c4ccccc4)ccc(-c4cccc(-c5ccc(-c6ccc(N(c7ccccc7)c7ccccc7)cc6)cc5)c4)c23)cc1]

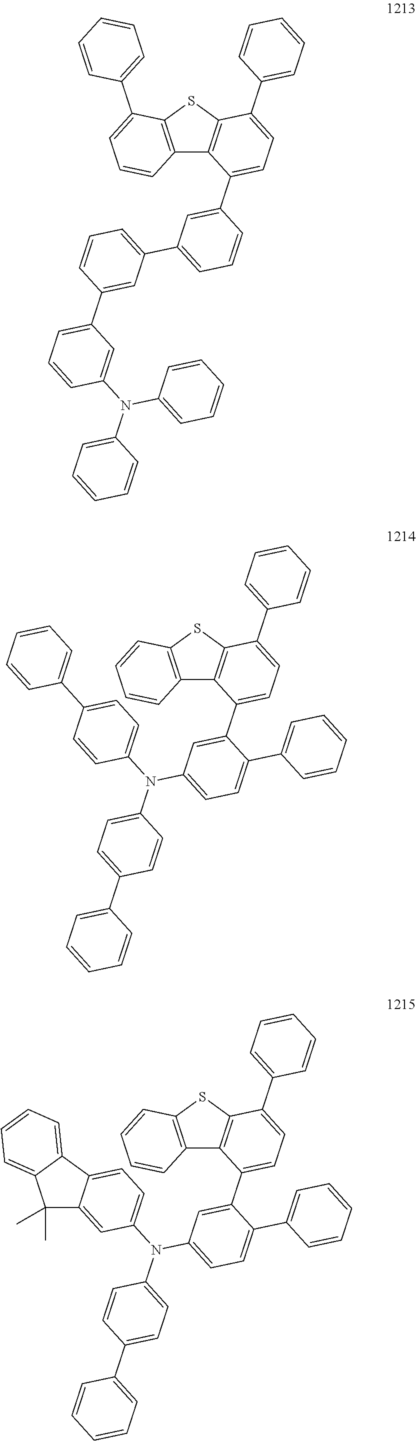 [SMILES: c1ccc(-c2cccc3c2sc2c(-c4ccccc4)ccc(-c4cccc(-c5cccc(-c6cccc(N(c7ccccc7)c7ccccc7)c6)c5)c4)c23)cc1 ; c1ccc(-c2ccc(N(c3ccc(-c4ccccc4)cc3)c3ccc(-c4ccccc4)c(-c4ccc(-c5ccccc5)c5sc6ccccc6c45)c3)cc2)cc1 ; CC1(C)c2ccccc2-c2ccc(N(c3ccc(-c4ccccc4)cc3)c3ccc(-c4ccccc4)c(-c4ccc(-c5ccccc5)c5sc6ccccc6c45)c3)cc21]

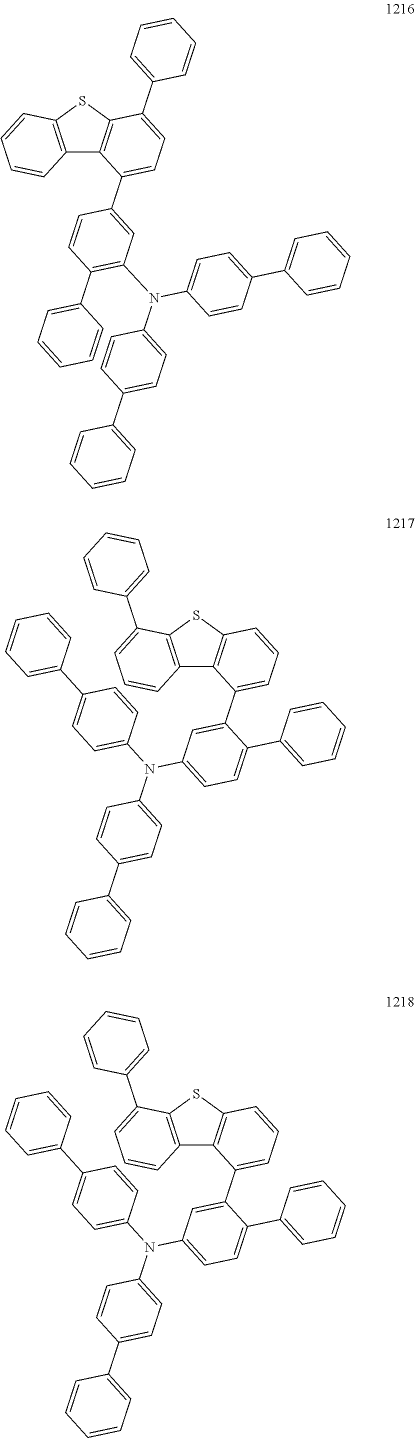

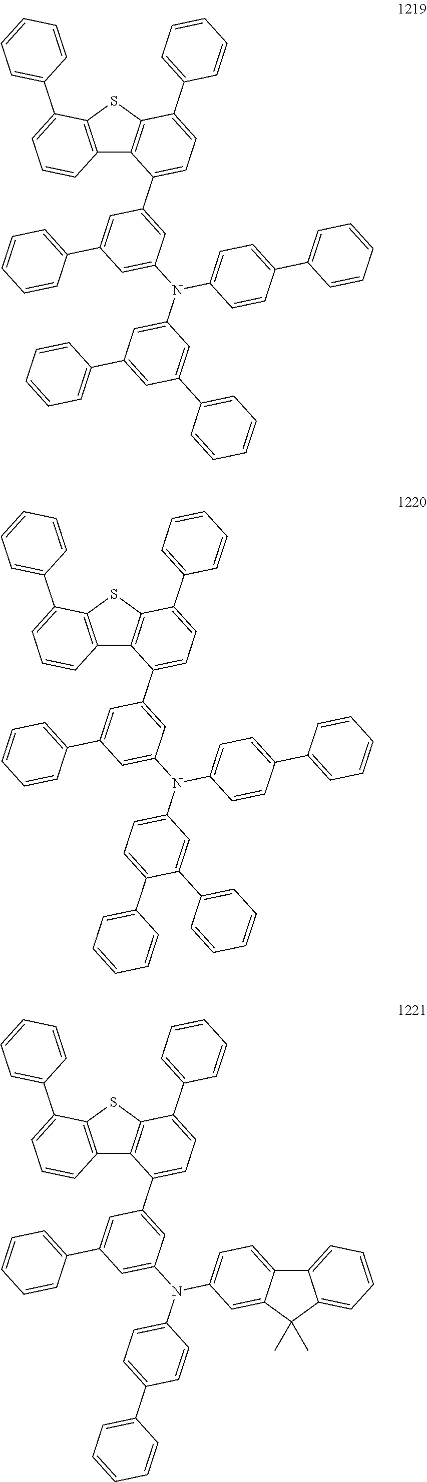 [SMILES: c1ccc(-c2ccc(N(c3cc(-c4ccccc4)cc(-c4ccccc4)c3)c3cc(-c4ccccc4)cc(-c4ccc(-c5ccccc5)c5sc6c(-c7ccccc7)cccc6c45)c3)cc2)cc1 ; c1ccc(-c2ccc(N(c3cc(-c4ccccc4)cc(-c4ccc(-c5ccccc5)c5sc6c(-c7ccccc7)cccc6c45)c3)c3ccc(-c4ccccc4)c(-c4ccccc4)c3)cc2)cc1 ; CC1(C)c2ccccc2-c2ccc(N(c3ccc(-c4ccccc4)cc3)c3cc(-c4ccccc4)cc(-c4ccc(-c5ccccc5)c5sc6c(-c7ccccc7)cccc6c45)c3)cc21]

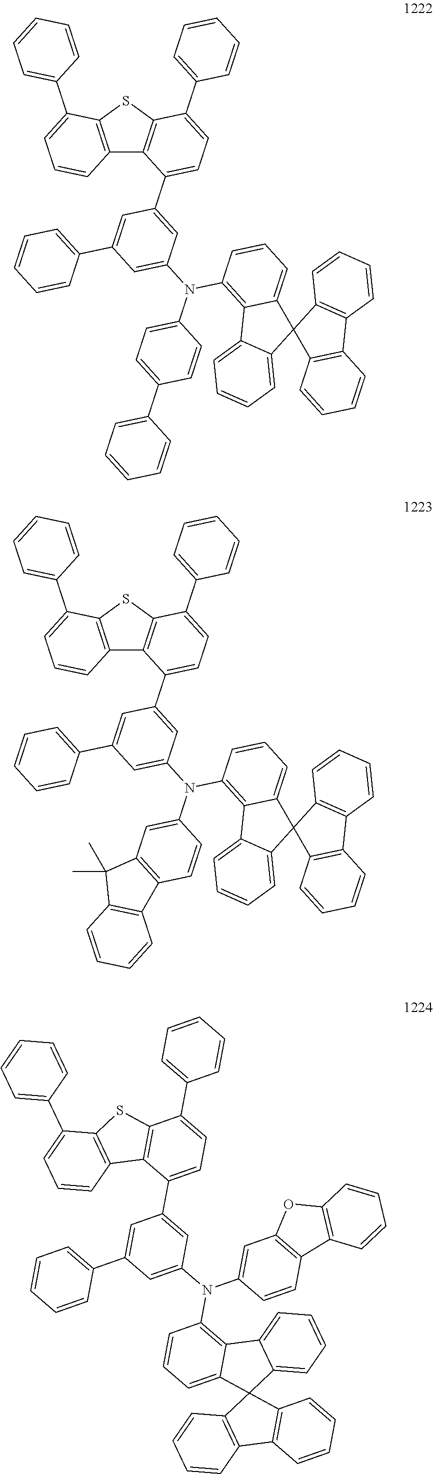 [SMILES: c1ccc(-c2ccc(N(c3cc(-c4ccccc4)cc(-c4ccc(-c5ccccc5)c5sc6c(-c7ccccc7)cccc6c45)c3)c3cccc4c3-c3ccccc3C43c4ccccc4-c4ccccc43)cc2)cc1 ; CC1(C)c2ccccc2-c2ccc(N(c3cc(-c4ccccc4)cc(-c4ccc(-c5ccccc5)c5sc6c(-c7ccccc7)cccc6c45)c3)c3cccc4c3-c3ccccc3C43c4ccccc4-c4ccccc43)cc21 ; c1ccc(-c2cc(-c3ccc(-c4ccccc4)c4sc5c(-c6ccccc6)cccc5c34)cc(N(c3ccc4c(c3)oc3ccccc34)c3cccc4c3-c3ccccc3C43c4ccccc4-c4ccccc43)c2)cc1]

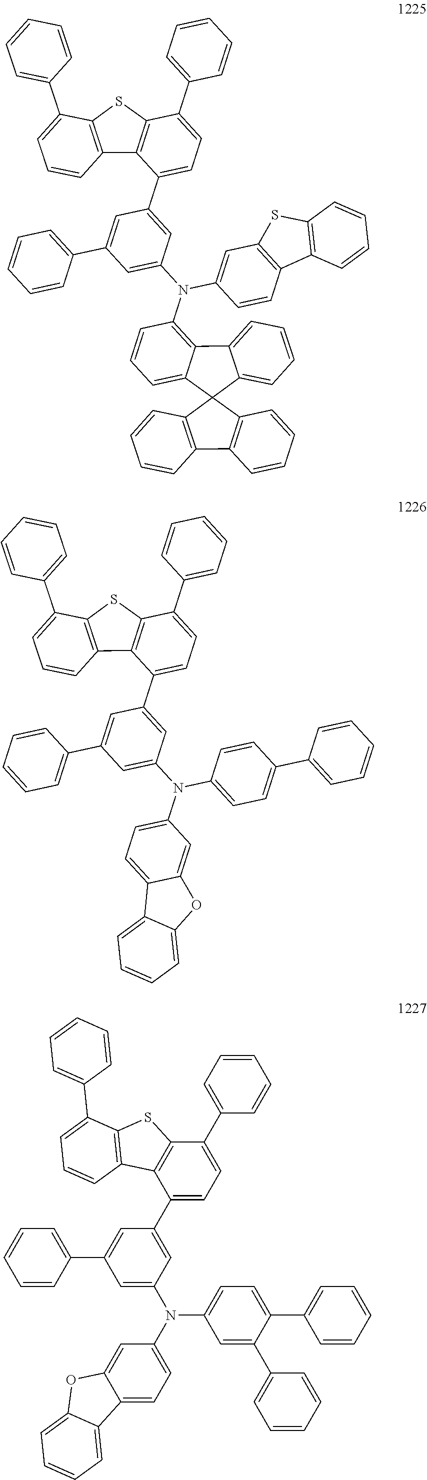 [SMILES: c1ccc(-c2cc(-c3ccc(-c4ccccc4)c4sc5c(-c6ccccc6)cccc5c34)cc(N(c3ccc4c(c3)sc3ccccc34)c3cccc4c3-c3ccccc3C43c4ccccc4-c4ccccc43)c2)cc1 ; c1ccc(-c2ccc(N(c3cc(-c4ccccc4)cc(-c4ccc(-c5ccccc5)c5sc6c(-c7ccccc7)cccc6c45)c3)c3ccc4c(c3)oc3ccccc34)cc2)cc1 ; c1ccc(-c2cc(-c3ccc(-c4ccccc4)c4sc5c(-c6ccccc6)cccc5c34)cc(N(c3ccc(-c4ccccc4)c(-c4ccccc4)c3)c3ccc4c(c3)oc3ccccc34)c2)cc1]

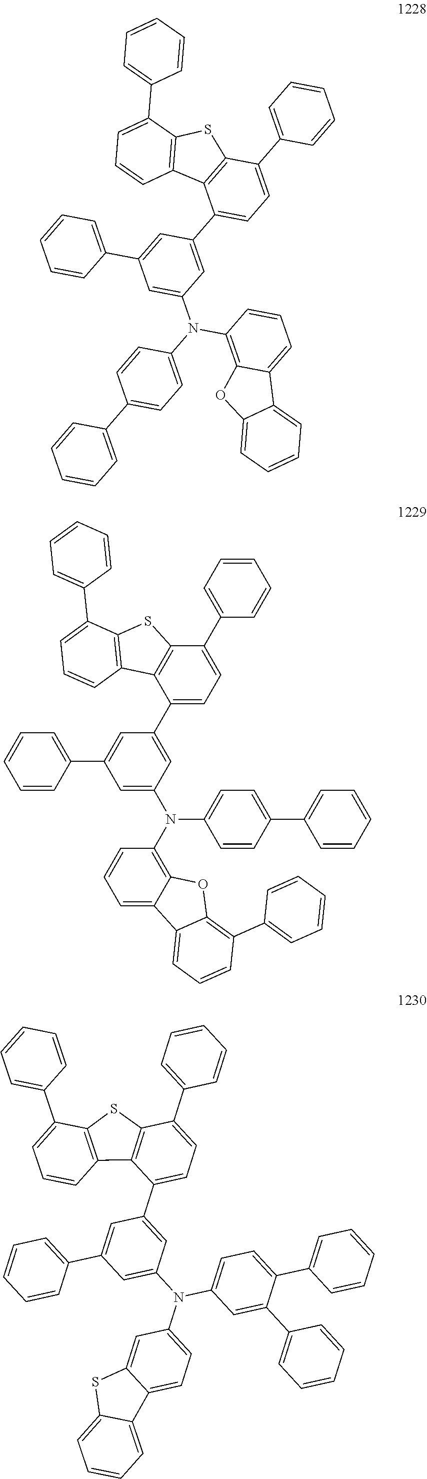

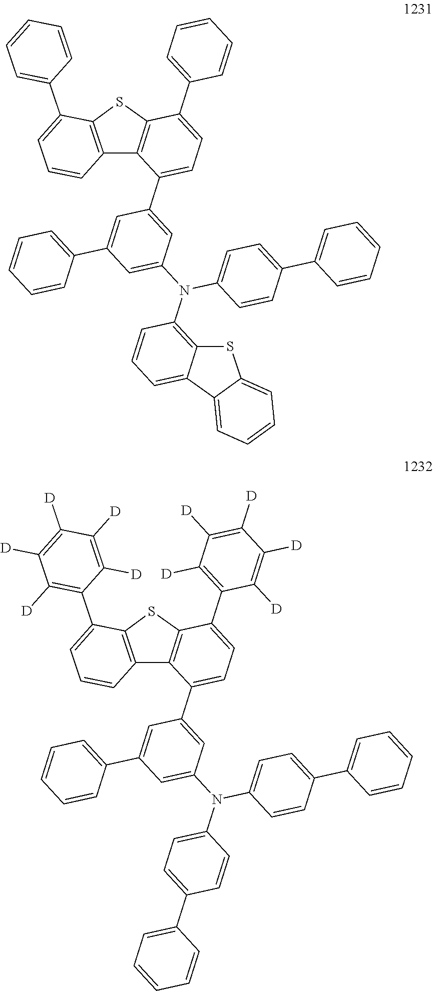 [SMILES: c1ccc(-c2ccc(N(c3cc(-c4ccccc4)cc(-c4ccc(-c5ccccc5)c5sc6c(-c7ccccc7)cccc6c45)c3)c3cccc4c3sc3ccccc34)cc2)cc1 ; [2H]c1c([2H])c([2H])c(-c2cccc3c2sc2c(-c4c([2H])c([2H])c([2H])c([2H])c4[2H])ccc(-c4cc(-c5ccccc5)cc(N(c5ccc(-c6ccccc6)cc5)c5ccc(-c6ccccc6)cc5)c4)c23)c([2H])c1[2H]]

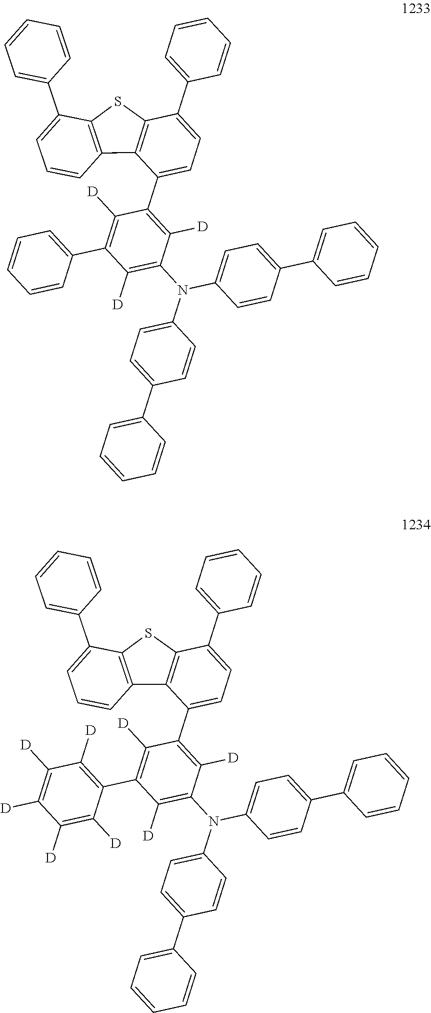 [SMILES: [2H]c1c(-c2ccccc2)c([2H])c(N(c2ccc(-c3ccccc3)cc2)c2ccc(-c3ccccc3)cc2)c([2H])c1-c1ccc(-c2ccccc2)c2sc3c(-c4ccccc4)cccc3c12 ; [2H]c1c([2H])c([2H])c(-c2c([2H])c(-c3ccc(-c4ccccc4)c4sc5c(-c6ccccc6)cccc5c34)c([2H])c(N(c3ccc(-c4ccccc4)cc3)c3ccc(-c4ccccc4)cc3)c2[2H])c([2H])c1[2H]]

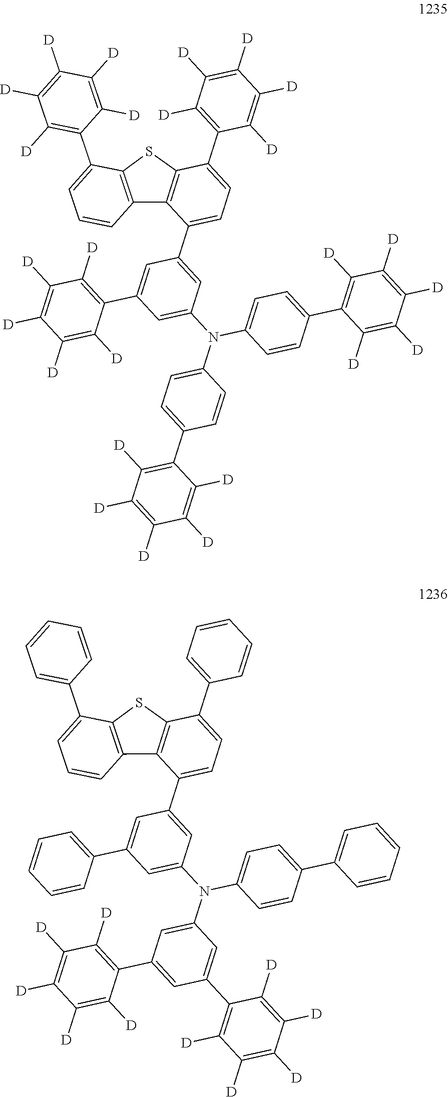 [SMILES: [2H]c1c([2H])c([2H])c(-c2ccc(N(c3ccc(-c4c([2H])c([2H])c([2H])c([2H])c4[2H])cc3)c3cc(-c4c([2H])c([2H])c([2H])c([2H])c4[2H])cc(-c4ccc(-c5c([2H])c([2H])c([2H])c([2H])c5[2H])c5sc6c(-c7c([2H])c([2H])c([2H])c([2H])c7[2H])cccc6c45)c3)cc2)c([2H])c1[2H] ; [2H]c1c([2H])c([2H])c(-c2cc(-c3c([2H])c([2H])c([2H])c([2H])c3[2H])cc(N(c3ccc(-c4ccccc4)cc3)c3cc(-c4ccccc4)cc(-c4ccc(-c5ccccc5)c5sc6c(-c7ccccc7)cccc6c45)c3)c2)c([2H])c1[2H]]

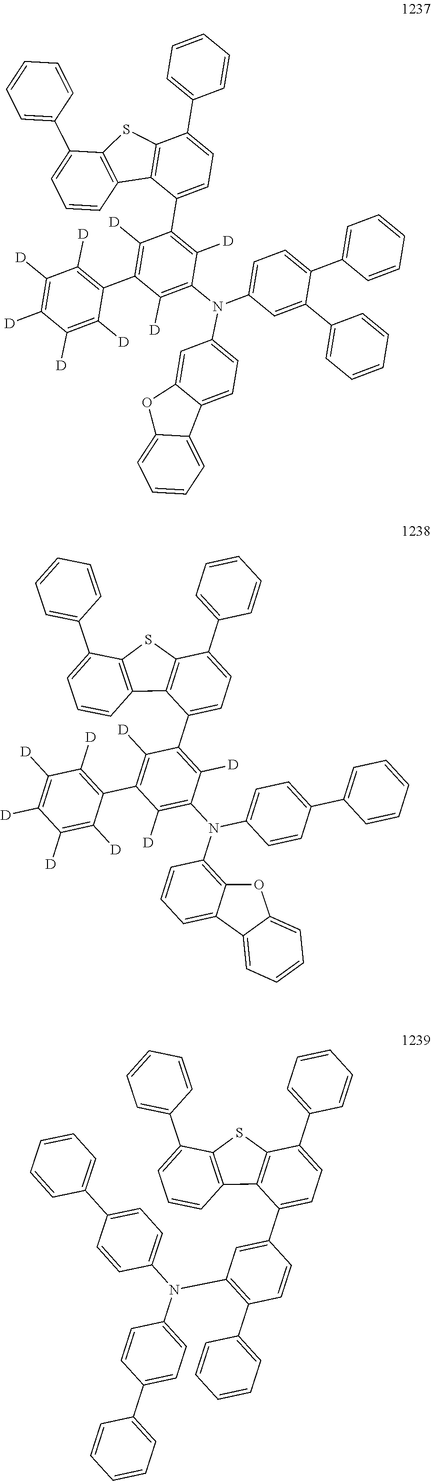 [SMILES: [2H]c1c([2H])c([2H])c(-c2c([2H])c(-c3ccc(-c4ccccc4)c4sc5c(-c6ccccc6)cccc5c34)c([2H])c(N(c3ccc(-c4ccccc4)c(-c4ccccc4)c3)c3ccc4c(c3)oc3ccccc34)c2[2H])c([2H])c1[2H] ; [2H]c1c([2H])c([2H])c(-c2c([2H])c(-c3ccc(-c4ccccc4)c4sc5c(-c6ccccc6)cccc5c34)c([2H])c(N(c3ccc(-c4ccccc4)cc3)c3cccc4c3oc3ccccc34)c2[2H])c([2H])c1[2H] ; c1ccc(-c2ccc(N(c3ccc(-c4ccccc4)cc3)c3cc(-c4ccc(-c5ccccc5)c5sc6c(-c7ccccc7)cccc6c45)ccc3-c3ccccc3)cc2)cc1]

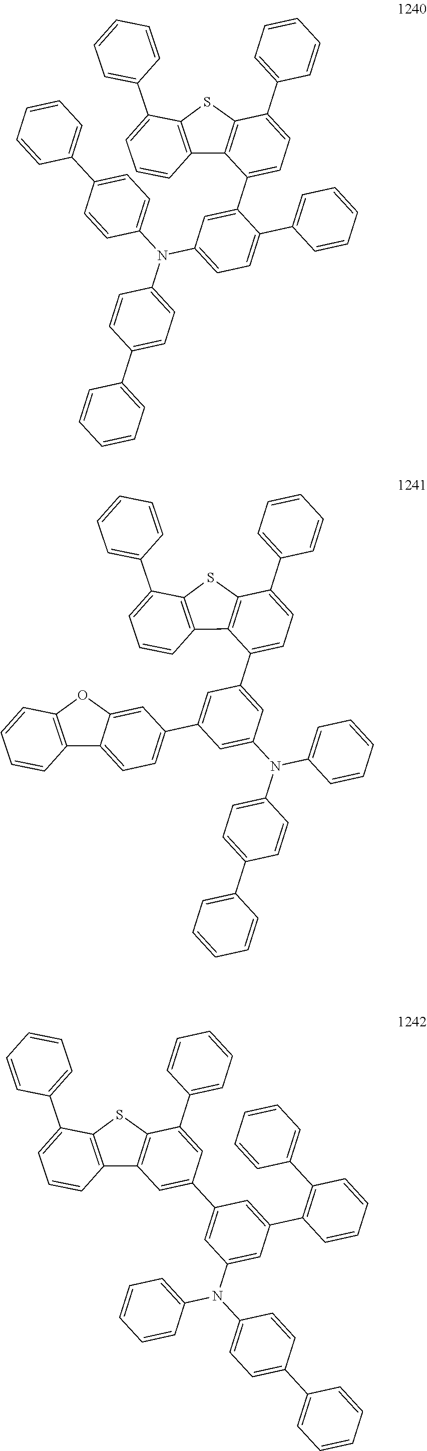 [SMILES: c1ccc(-c2ccc(N(c3ccc(-c4ccccc4)cc3)c3ccc(-c4ccccc4)c(-c4ccc(-c5ccccc5)c5sc6c(-c7ccccc7)cccc6c45)c3)cc2)cc1 ; c1ccc(-c2ccc(N(c3ccccc3)c3cc(-c4ccc5c(c4)oc4ccccc45)cc(-c4ccc(-c5ccccc5)c5sc6c(-c7ccccc7)cccc6c45)c3)cc2)cc1 ; c1ccc(-c2ccc(N(c3ccccc3)c3cc(-c4cc(-c5ccccc5)c5sc6c(-c7ccccc7)cccc6c5c4)cc(-c4ccccc4-c4ccccc4)c3)cc2)cc1]

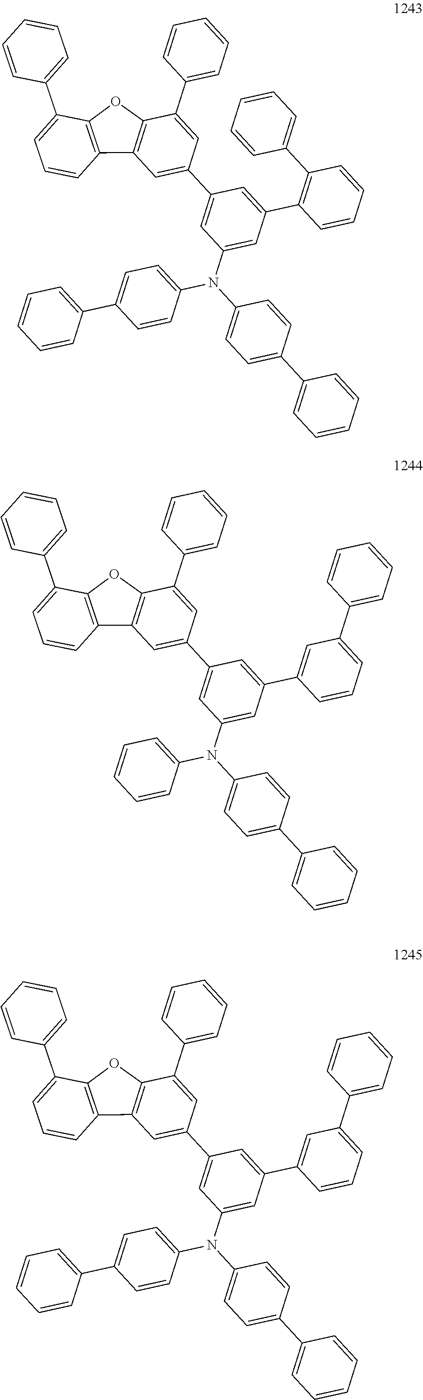 [SMILES: c1ccc(-c2ccc(N(c3ccc(-c4ccccc4)cc3)c3cc(-c4cc(-c5ccccc5)c5oc6c(-c7ccccc7)cccc6c5c4)cc(-c4ccccc4-c4ccccc4)c3)cc2)cc1 ; c1ccc(-c2ccc(N(c3ccccc3)c3cc(-c4cccc(-c5ccccc5)c4)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4cccc(-c5ccccc5)c4)cc(-c4cc(-c5ccccc5)c5oc6c(-c7ccccc7)cccc6c5c4)c3)cc2)cc1]

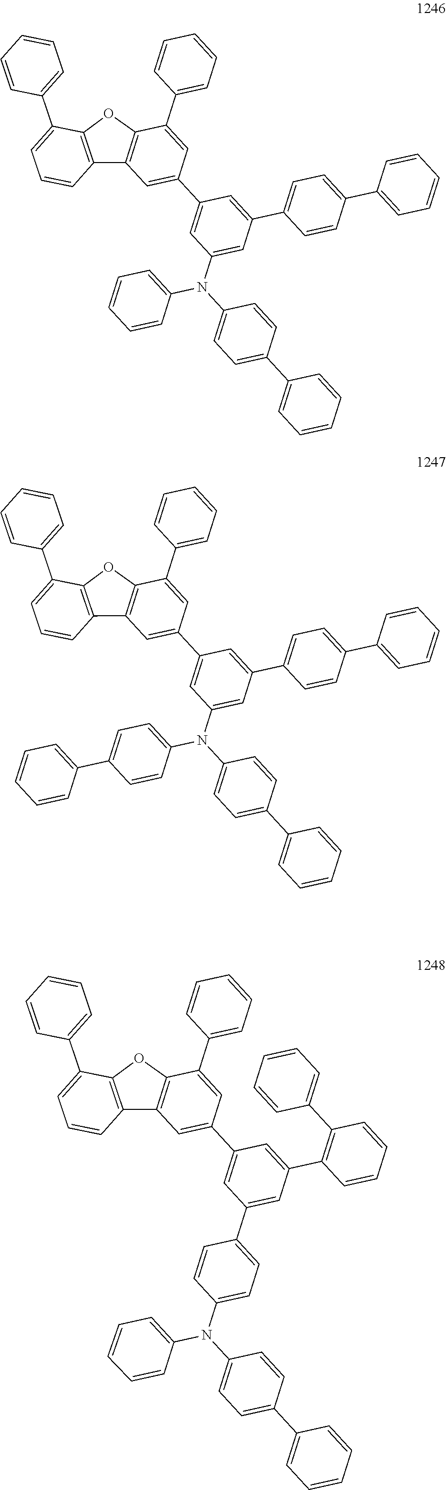 [SMILES: c1ccc(-c2ccc(-c3cc(-c4cc(-c5ccccc5)c5oc6c(-c7ccccc7)cccc6c5c4)cc(N(c4ccccc4)c4ccc(-c5ccccc5)cc4)c3)cc2)cc1 ; c1ccc(-c2ccc(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)cc2)cc1 ; c1ccc(-c2ccc(N(c3ccccc3)c3ccc(-c4cc(-c5cc(-c6ccccc6)c6oc7c(-c8ccccc8)cccc7c6c5)cc(-c5ccccc5-c5ccccc5)c4)cc3)cc2)cc1]

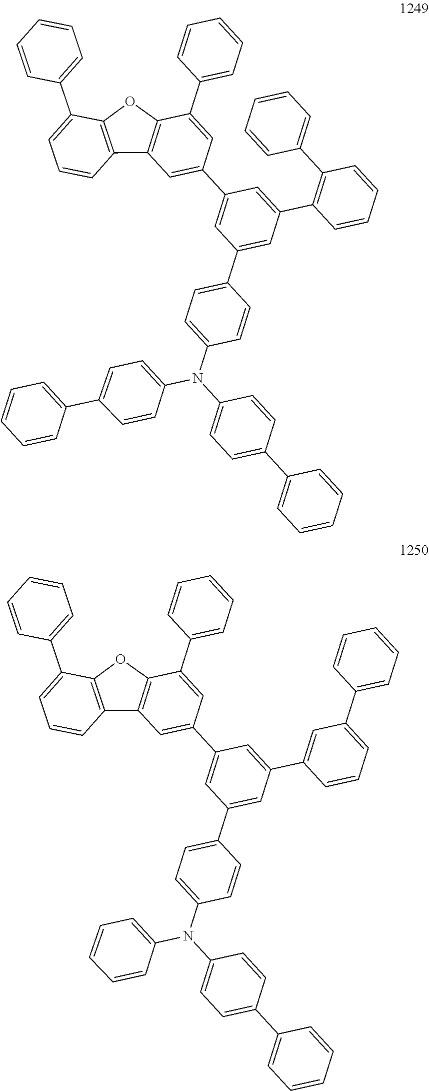 [SMILES: c1ccc(-c2ccc(N(c3ccc(-c4ccccc4)cc3)c3ccc(-c4cc(-c5cc(-c6ccccc6)c6oc7c(-c8ccccc8)cccc7c6c5)cc(-c5ccccc5-c5ccccc5)c4)cc3)cc2)cc1 ; c1ccc(-c2ccc(N(c3ccccc3)c3ccc(-c4cc(-c5cccc(-c6ccccc6)c5)cc(-c5cc(-c6ccccc6)c6oc7c(-c8ccccc8)cccc7c6c5)c4)cc3)cc2)cc1]

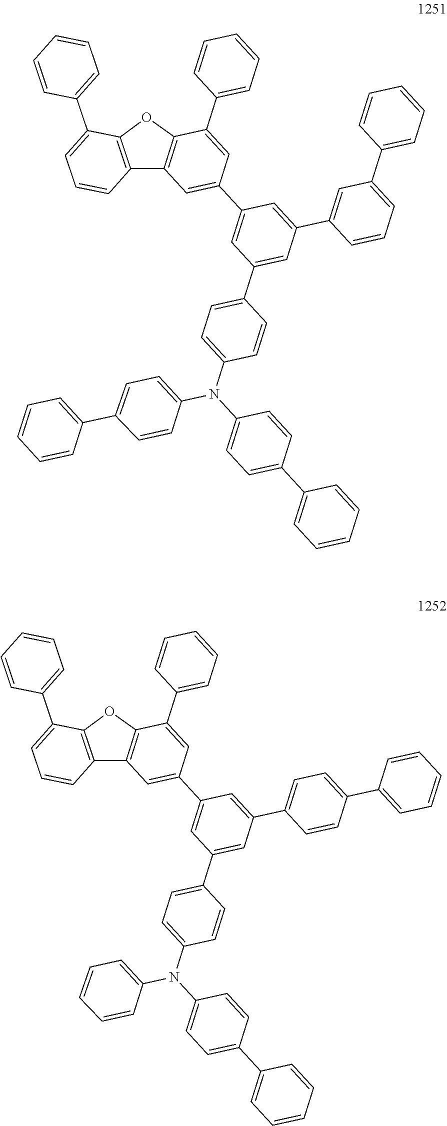 [SMILES: c1ccc(-c2ccc(N(c3ccc(-c4ccccc4)cc3)c3ccc(-c4cc(-c5cccc(-c6ccccc6)c5)cc(-c5cc(-c6ccccc6)c6oc7c(-c8ccccc8)cccc7c6c5)c4)cc3)cc2)cc1 ; c1ccc(-c2ccc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)cc2)cc1]

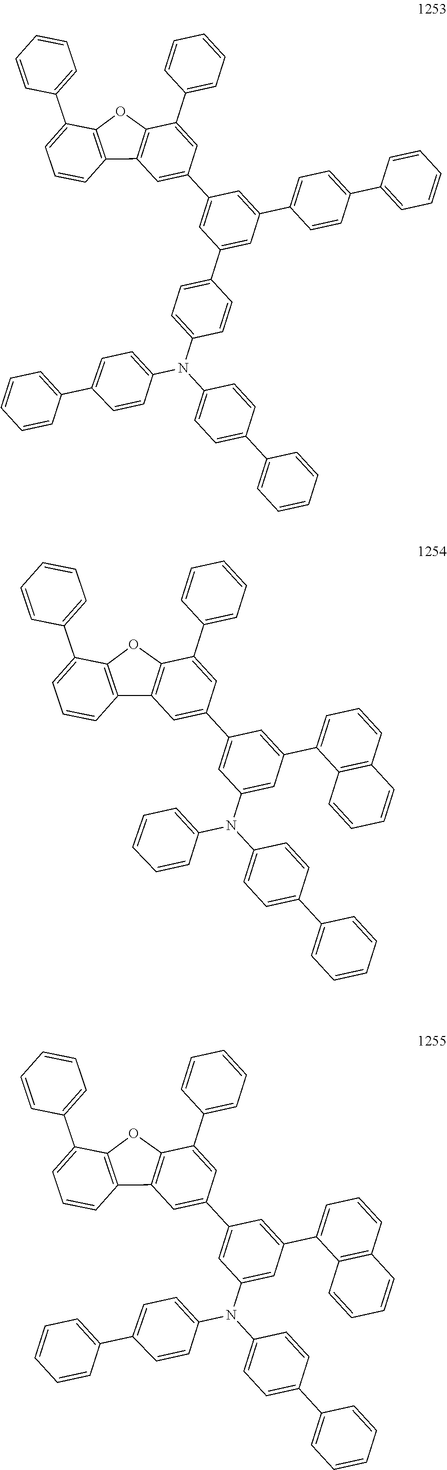 [SMILES: c1ccc(-c2ccc(-c3cc(-c4ccc(N(c5ccc(-c6ccccc6)cc5)c5ccc(-c6ccccc6)cc5)cc4)cc(-c4cc(-c5ccccc5)c5oc6c(-c7ccccc7)cccc6c5c4)c3)cc2)cc1 ; c1ccc(-c2ccc(N(c3ccccc3)c3cc(-c4cc(-c5ccccc5)c5oc6c(-c7ccccc7)cccc6c5c4)cc(-c4cccc5ccccc45)c3)cc2)cc1 ; c1ccc(-c2ccc(N(c3ccc(-c4ccccc4)cc3)c3cc(-c4cc(-c5ccccc5)c5oc6c(-c7ccccc7)cccc6c5c4)cc(-c4cccc5ccccc45)c3)cc2)cc1]

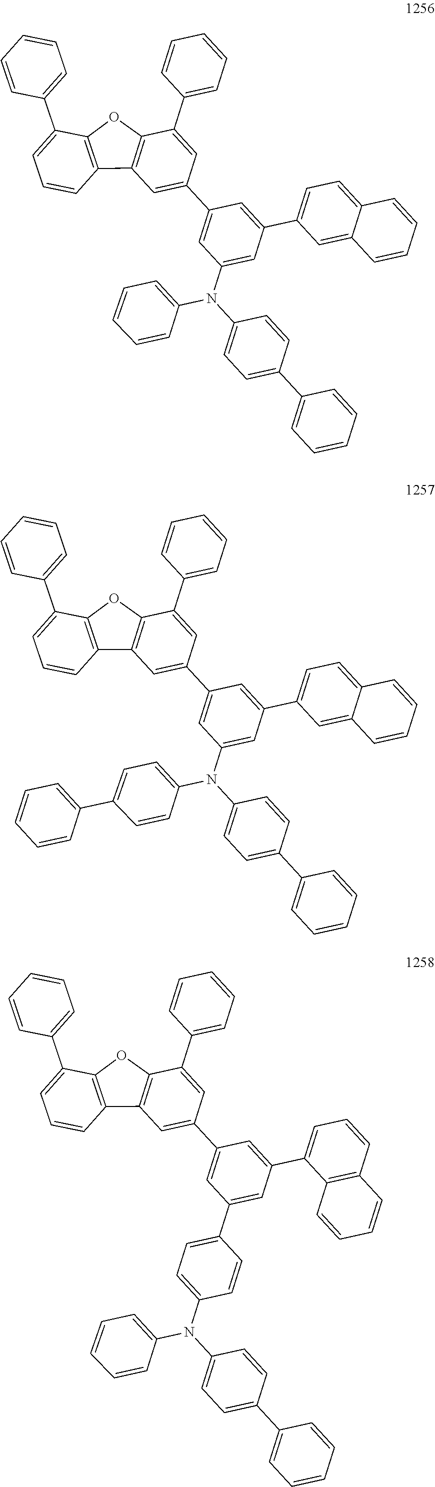 [SMILES: c1ccc(-c2ccc(N(c3ccccc3)c3cc(-c4ccc5ccccc5c4)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4ccc5ccccc5c4)cc(-c4cc(-c5ccccc5)c5oc6c(-c7ccccc7)cccc6c5c4)c3)cc2)cc1 ; c1ccc(-c2ccc(N(c3ccccc3)c3ccc(-c4cc(-c5cc(-c6ccccc6)c6oc7c(-c8ccccc8)cccc7c6c5)cc(-c5cccc6ccccc56)c4)cc3)cc2)cc1]

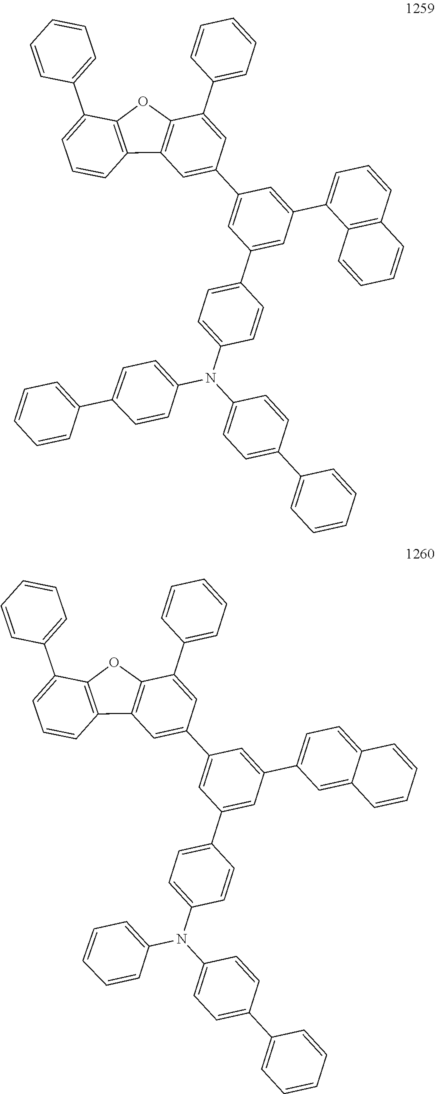 [SMILES: c1ccc(-c2ccc(N(c3ccc(-c4ccccc4)cc3)c3ccc(-c4cc(-c5cc(-c6ccccc6)c6oc7c(-c8ccccc8)cccc7c6c5)cc(-c5cccc6ccccc56)c4)cc3)cc2)cc1 ; c1ccc(-c2ccc(N(c3ccccc3)c3ccc(-c4cc(-c5ccc6ccccc6c5)cc(-c5cc(-c6ccccc6)c6oc7c(-c8ccccc8)cccc7c6c5)c4)cc3)cc2)cc1]

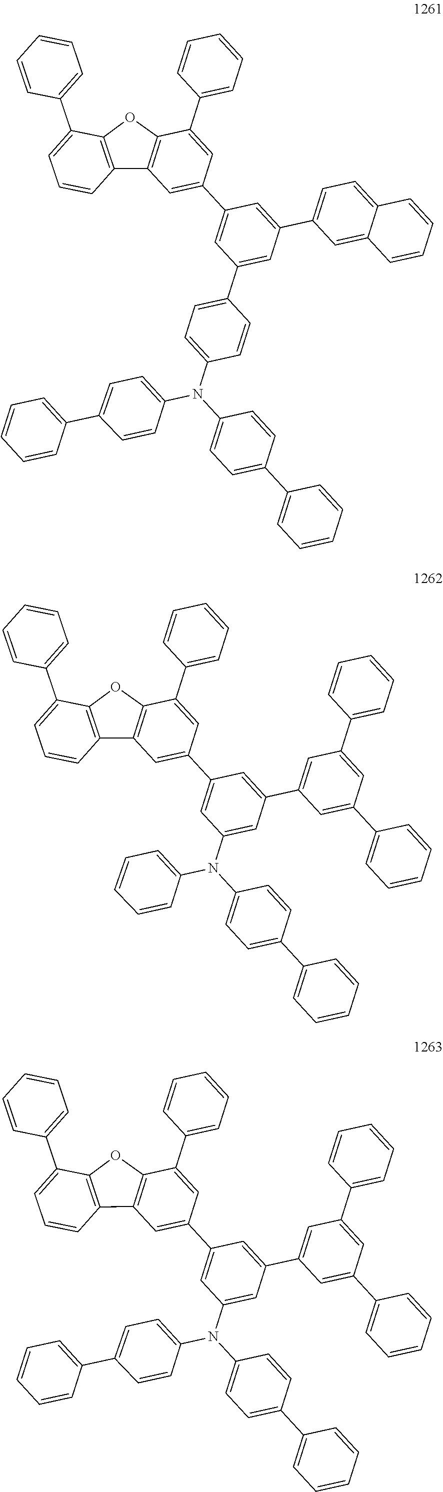 [SMILES: c1ccc(-c2ccc(N(c3ccc(-c4ccccc4)cc3)c3ccc(-c4cc(-c5ccc6ccccc6c5)cc(-c5cc(-c6ccccc6)c6oc7c(-c8ccccc8)cccc7c6c5)c4)cc3)cc2)cc1 ; c1ccc(-c2ccc(N(c3ccccc3)c3cc(-c4cc(-c5ccccc5)cc(-c5ccccc5)c4)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4cc(-c5ccccc5)cc(-c5ccccc5)c4)cc(-c4cc(-c5ccccc5)c5oc6c(-c7ccccc7)cccc6c5c4)c3)cc2)cc1]

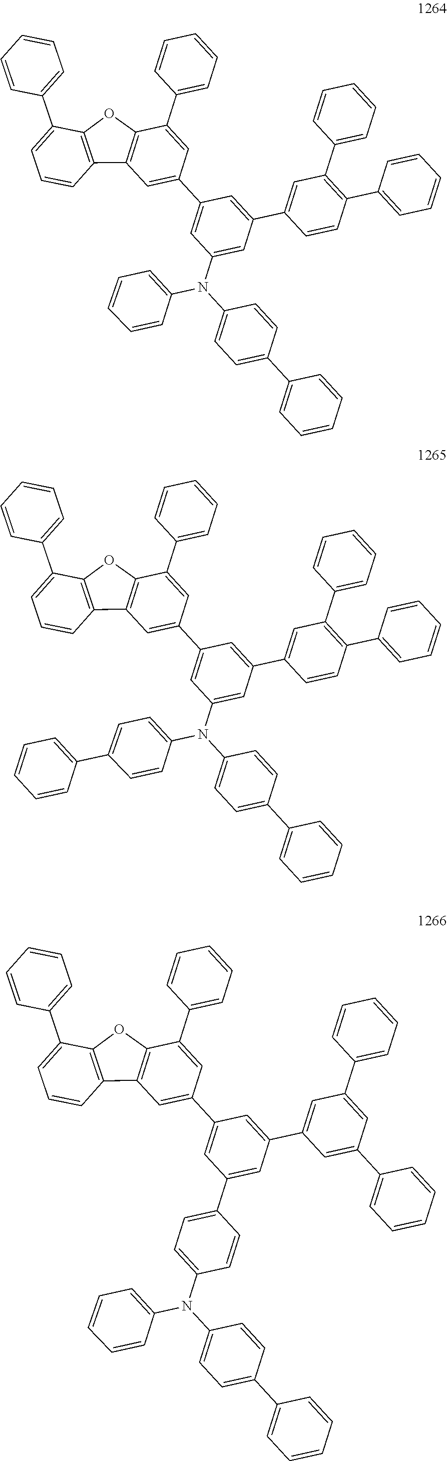 [SMILES: c1ccc(-c2ccc(N(c3ccccc3)c3cc(-c4ccc(-c5ccccc5)c(-c5ccccc5)c4)cc(-c4cc(-c5ccccc5)c5oc6c(-c7ccccc7)cccc6c5c4)c3)cc2)cc1 ; c1ccc(-c2ccc(N(c3ccc(-c4ccccc4)cc3)c3cc(-c4ccc(-c5ccccc5)c(-c5ccccc5)c4)cc(-c4cc(-c5ccccc5)c5oc6c(-c7ccccc7)cccc6c5c4)c3)cc2)cc1 ; c1ccc(-c2ccc(N(c3ccccc3)c3ccc(-c4cc(-c5cc(-c6ccccc6)cc(-c6ccccc6)c5)cc(-c5cc(-c6ccccc6)c6oc7c(-c8ccccc8)cccc7c6c5)c4)cc3)cc2)cc1]

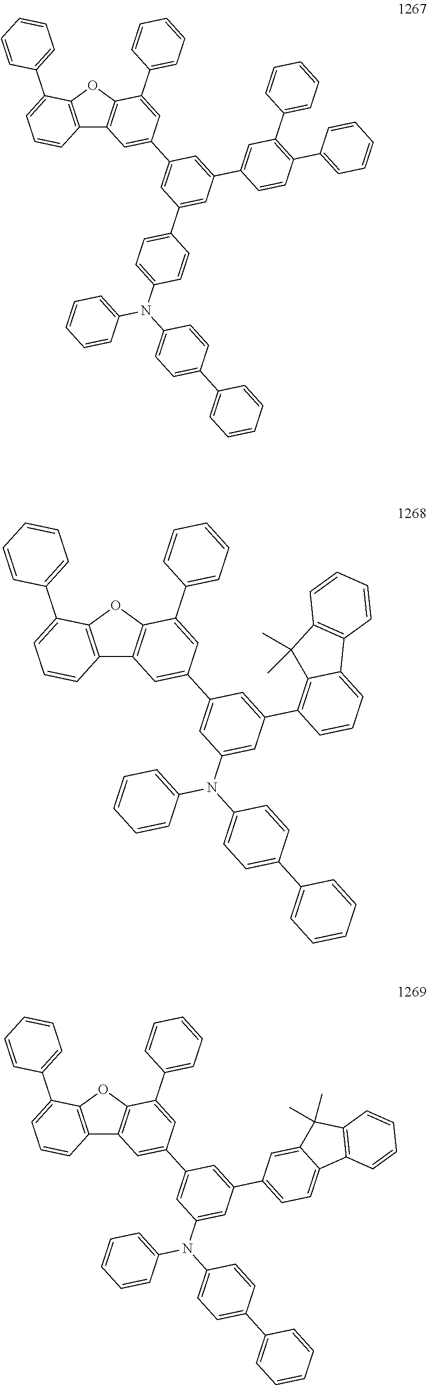 [SMILES: c1ccc(-c2ccc(N(c3ccccc3)c3ccc(-c4cc(-c5ccc(-c6ccccc6)c(-c6ccccc6)c5)cc(-c5cc(-c6ccccc6)c6oc7c(-c8ccccc8)cccc7c6c5)c4)cc3)cc2)cc1 ; CC1(C)c2ccccc2-c2cccc(-c3cc(-c4cc(-c5ccccc5)c5oc6c(-c7ccccc7)cccc6c5c4)cc(N(c4ccccc4)c4ccc(-c5ccccc5)cc4)c3)c21 ; CC1(C)c2ccccc2-c2ccc(-c3cc(-c4cc(-c5ccccc5)c5oc6c(-c7ccccc7)cccc6c5c4)cc(N(c4ccccc4)c4ccc(-c5ccccc5)cc4)c3)cc21]

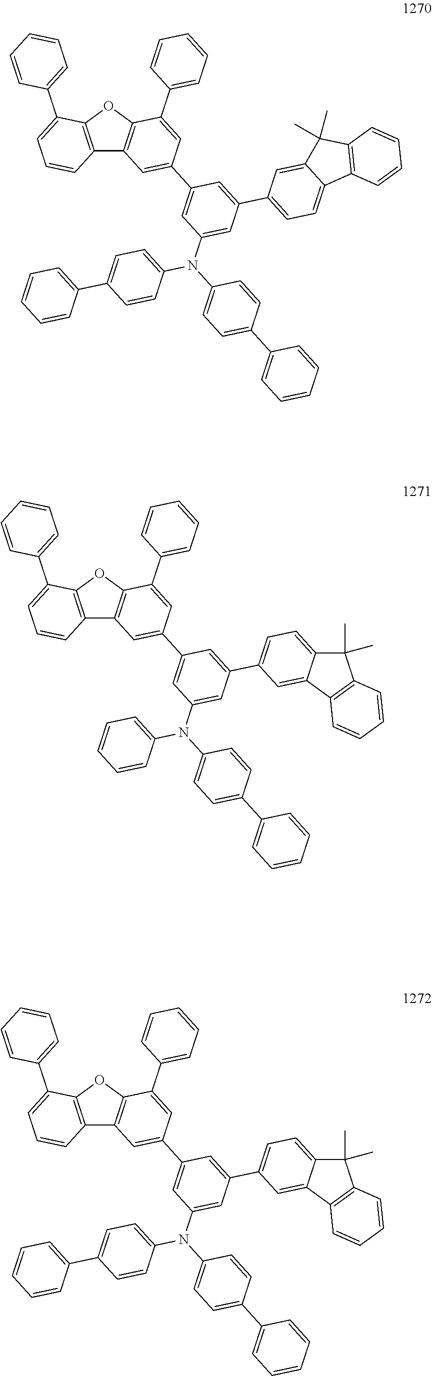 [SMILES: CC1(C)c2ccccc2-c2ccc(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)cc21 ; CC1(C)c2ccccc2-c2cc(-c3cc(-c4cc(-c5ccccc5)c5oc6c(-c7ccccc7)cccc6c5c4)cc(N(c4ccccc4)c4ccc(-c5ccccc5)cc4)c3)ccc21 ; CC1(C)c2ccccc2-c2cc(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)ccc21]

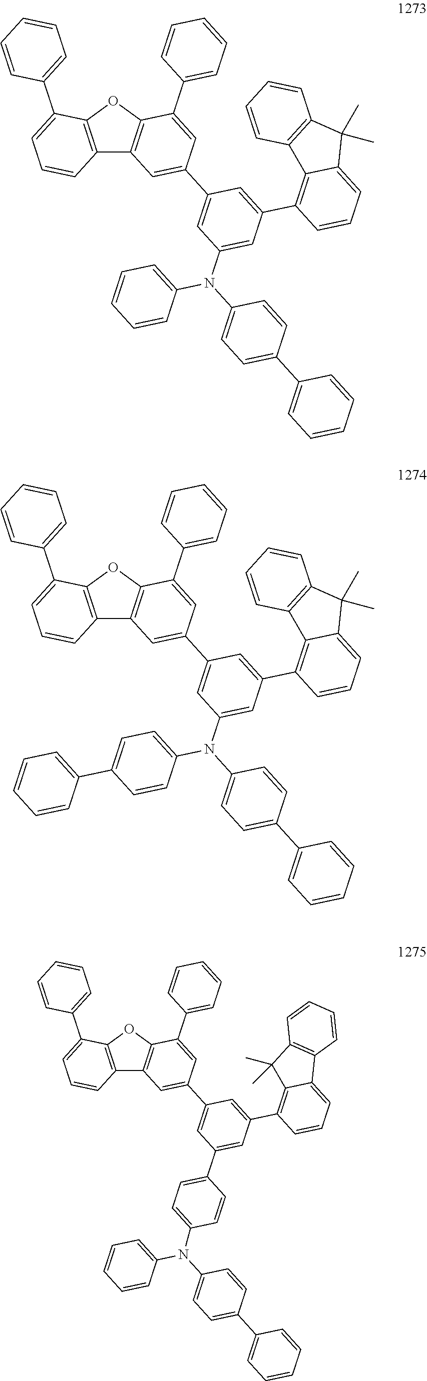 [SMILES: CC1(C)c2ccccc2-c2c(-c3cc(-c4cc(-c5ccccc5)c5oc6c(-c7ccccc7)cccc6c5c4)cc(N(c4ccccc4)c4ccc(-c5ccccc5)cc4)c3)cccc21 ; CC1(C)c2ccccc2-c2c(-c3cc(-c4cc(-c5ccccc5)c5oc6c(-c7ccccc7)cccc6c5c4)cc(N(c4ccc(-c5ccccc5)cc4)c4ccc(-c5ccccc5)cc4)c3)cccc21 ; CC1(C)c2ccccc2-c2cccc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)c21]

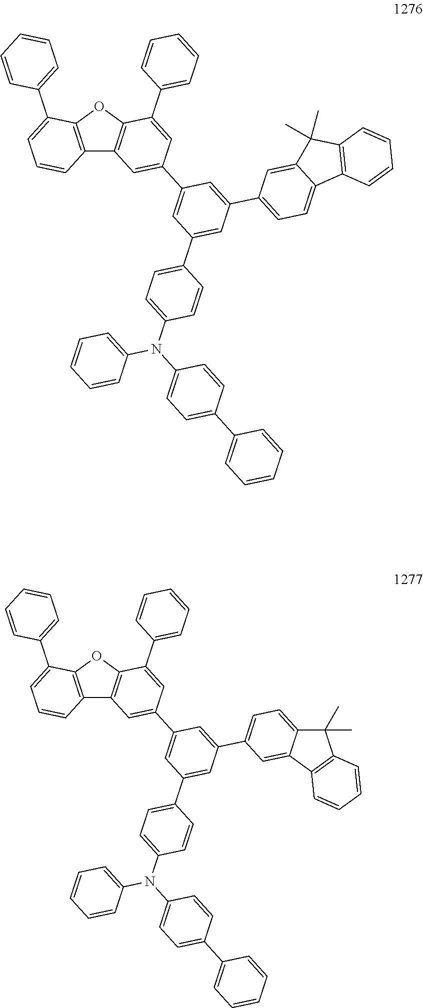 [SMILES: CC1(C)c2ccccc2-c2ccc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)cc21 ; CC1(C)c2ccccc2-c2cc(-c3cc(-c4ccc(N(c5ccccc5)c5ccc(-c6ccccc6)cc5)cc4)cc(-c4cc(-c5ccccc5)c5oc6c(-c7ccccc7)cccc6c5c4)c3)ccc21]

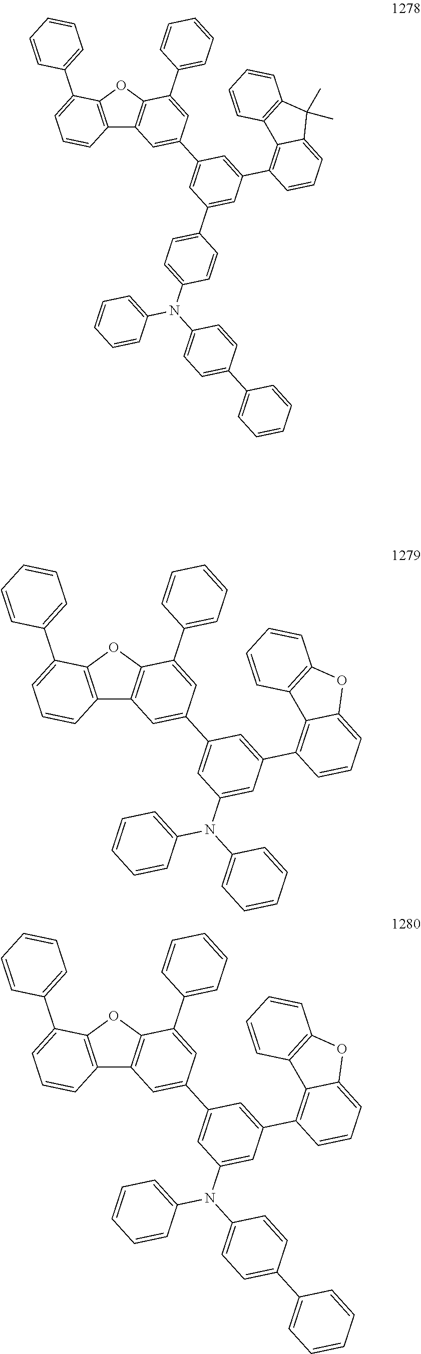

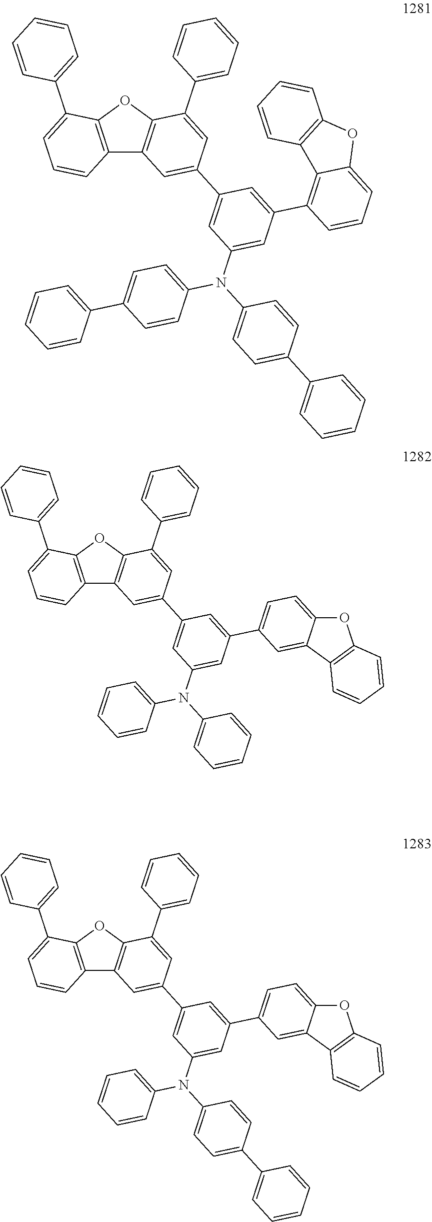 [SMILES: c1ccc(-c2ccc(N(c3ccc(-c4ccccc4)cc3)c3cc(-c4cc(-c5ccccc5)c5oc6c(-c7ccccc7)cccc6c5c4)cc(-c4cccc5oc6ccccc6c45)c3)cc2)cc1 ; c1ccc(-c2cccc3c2oc2c(-c4ccccc4)cc(-c4cc(-c5ccc6oc7ccccc7c6c5)cc(N(c5ccccc5)c5ccccc5)c4)cc23)cc1 ; c1ccc(-c2ccc(N(c3ccccc3)c3cc(-c4ccc5oc6ccccc6c5c4)cc(-c4cc(-c5ccccc5)c5oc6c(-c7ccccc7)cccc6c5c4)c3)cc2)cc1]

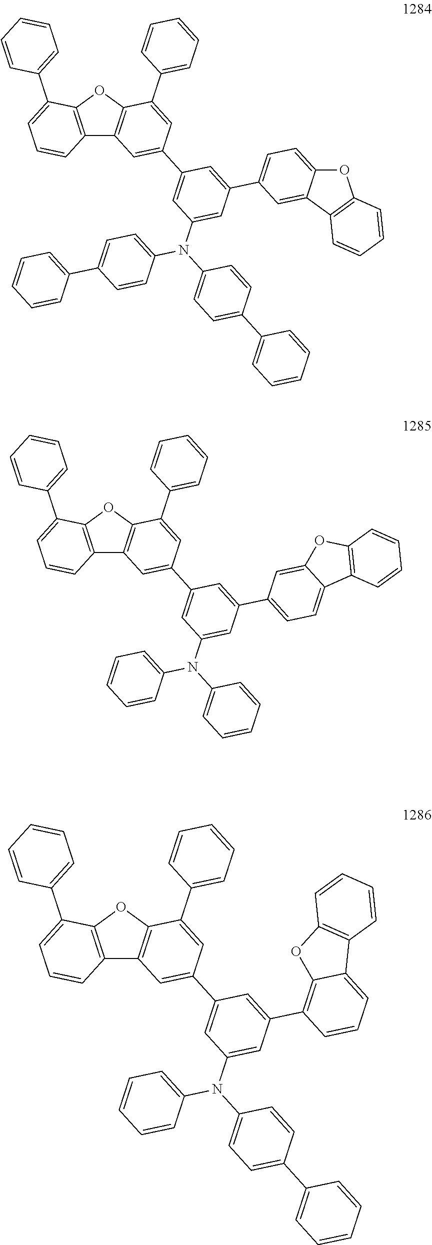 [SMILES: c1ccc(-c2ccc(N(c3ccc(-c4ccccc4)cc3)c3cc(-c4ccc5oc6ccccc6c5c4)cc(-c4cc(-c5ccccc5)c5oc6c(-c7ccccc7)cccc6c5c4)c3)cc2)cc1 ; c1ccc(-c2cccc3c2oc2c(-c4ccccc4)cc(-c4cc(-c5ccc6c(c5)oc5ccccc56)cc(N(c5ccccc5)c5ccccc5)c4)cc23)cc1 ; c1ccc(-c2ccc(N(c3ccccc3)c3cc(-c4cc(-c5ccccc5)c5oc6c(-c7ccccc7)cccc6c5c4)cc(-c4cccc5c4oc4ccccc45)c3)cc2)cc1]

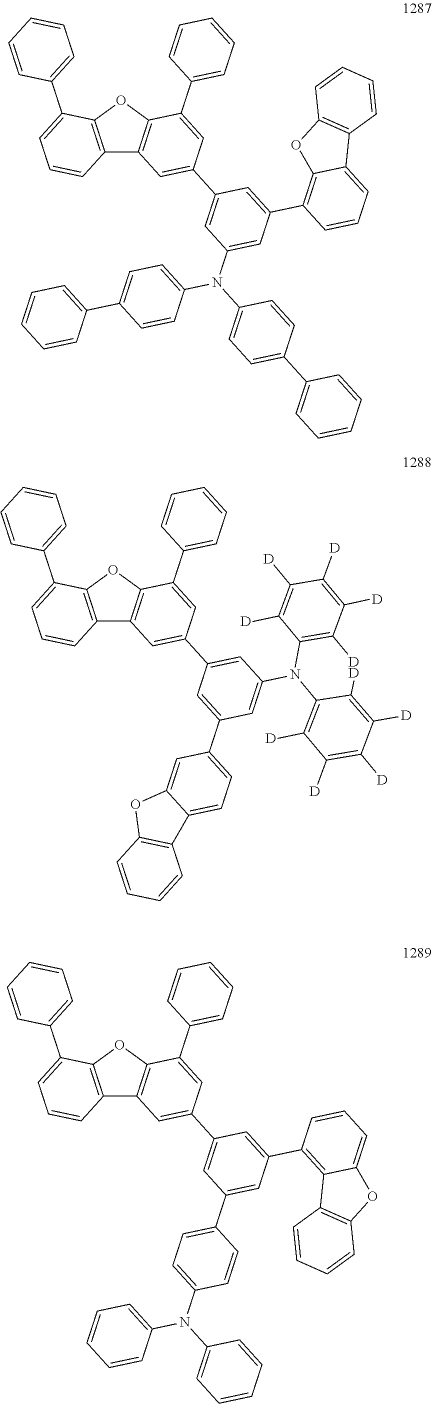

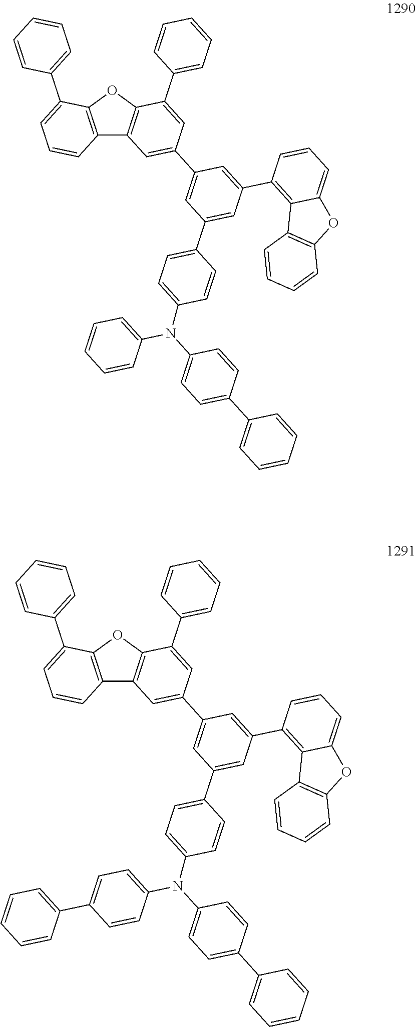 [SMILES: c1ccc(-c2ccc(N(c3ccccc3)c3ccc(-c4cc(-c5cc(-c6ccccc6)c6oc7c(-c8ccccc8)cccc7c6c5)cc(-c5cccc6oc7ccccc7c56)c4)cc3)cc2)cc1 ; c1ccc(-c2ccc(N(c3ccc(-c4ccccc4)cc3)c3ccc(-c4cc(-c5cc(-c6ccccc6)c6oc7c(-c8ccccc8)cccc7c6c5)cc(-c5cccc6oc7ccccc7c56)c4)cc3)cc2)cc1]

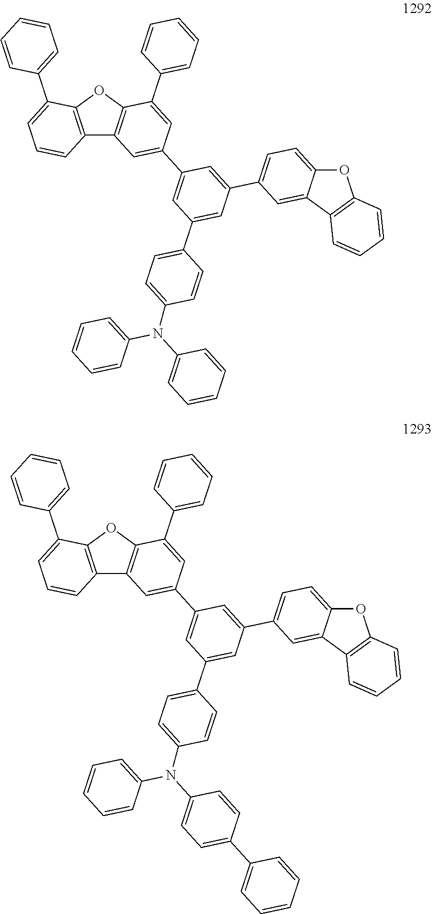 [SMILES: c1ccc(-c2cccc3c2oc2c(-c4ccccc4)cc(-c4cc(-c5ccc(N(c6ccccc6)c6ccccc6)cc5)cc(-c5ccc6oc7ccccc7c6c5)c4)cc23)cc1 ; c1ccc(-c2ccc(N(c3ccccc3)c3ccc(-c4cc(-c5ccc6oc7ccccc7c6c5)cc(-c5cc(-c6ccccc6)c6oc7c(-c8ccccc8)cccc7c6c5)c4)cc3)cc2)cc1]

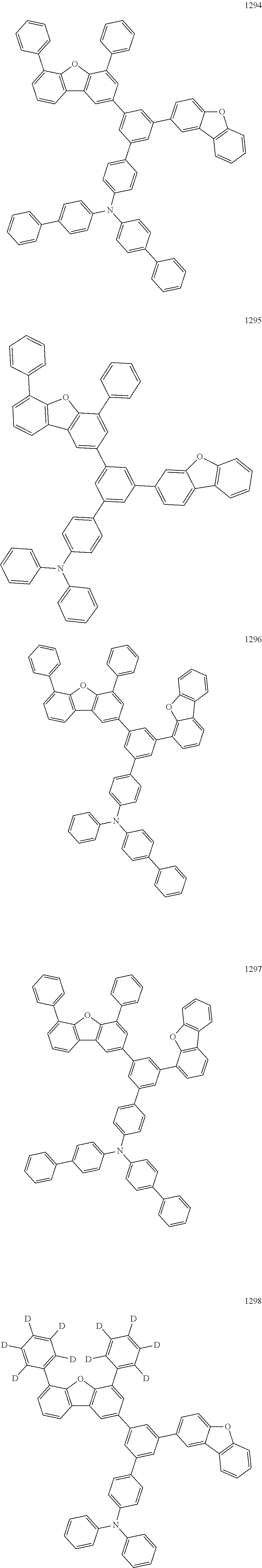

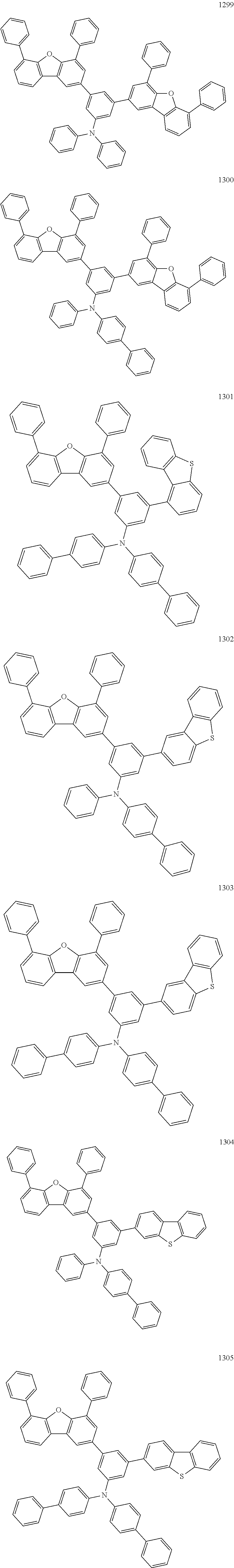

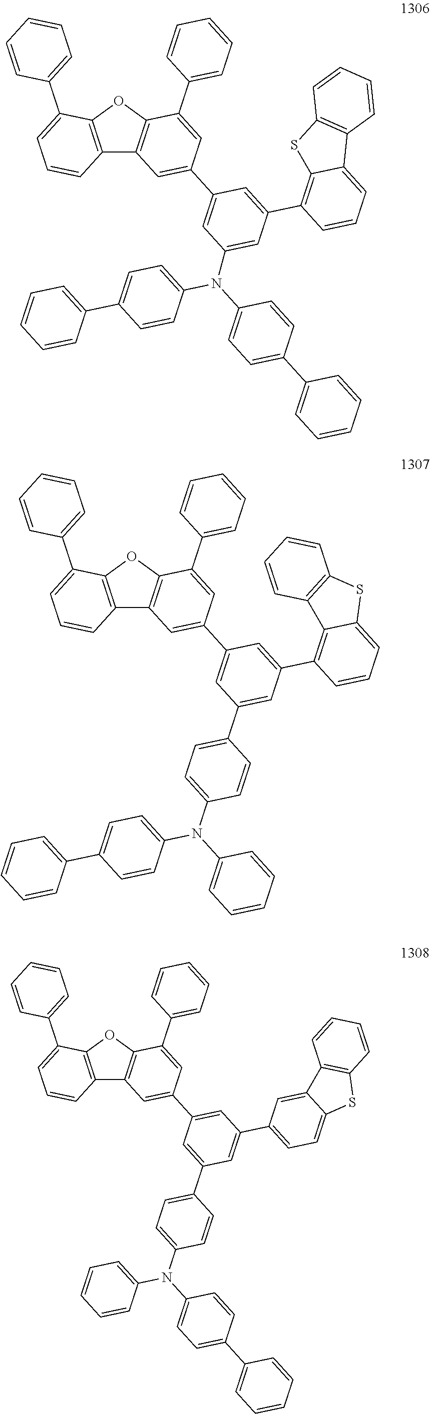 [SMILES: c1ccc(-c2ccc(N(c3ccc(-c4ccccc4)cc3)c3cc(-c4cc(-c5ccccc5)c5oc6c(-c7ccccc7)cccc6c5c4)cc(-c4cccc5c4sc4ccccc45)c3)cc2)cc1 ; c1ccc(-c2ccc(N(c3ccccc3)c3ccc(-c4cc(-c5cc(-c6ccccc6)c6oc7c(-c8ccccc8)cccc7c6c5)cc(-c5cccc6sc7ccccc7c56)c4)cc3)cc2)cc1 ; c1ccc(-c2ccc(N(c3ccccc3)c3ccc(-c4cc(-c5ccc6sc7ccccc7c6c5)cc(-c5cc(-c6ccccc6)c6oc7c(-c8ccccc8)cccc7c6c5)c4)cc3)cc2)cc1]

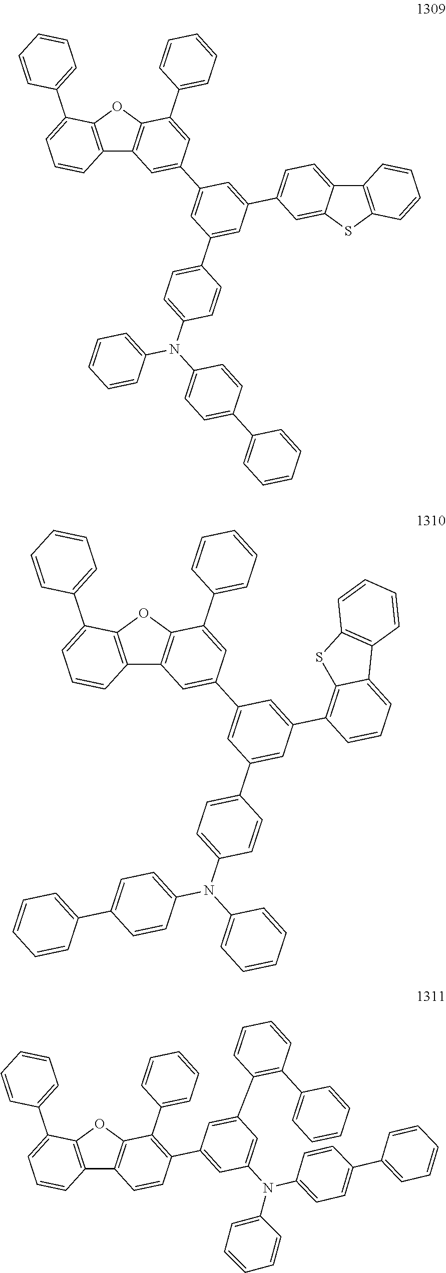 [SMILES: c1ccc(-c2ccc(N(c3ccccc3)c3ccc(-c4cc(-c5ccc6c(c5)sc5ccccc56)cc(-c5cc(-c6ccccc6)c6oc7c(-c8ccccc8)cccc7c6c5)c4)cc3)cc2)cc1 ; c1ccc(-c2ccc(N(c3ccccc3)c3ccc(-c4cc(-c5cc(-c6ccccc6)c6oc7c(-c8ccccc8)cccc7c6c5)cc(-c5cccc6c5sc5ccccc56)c4)cc3)cc2)cc1 ; c1ccc(-c2ccc(N(c3ccccc3)c3cc(-c4ccccc4-c4ccccc4)cc(-c4ccc5c(oc6c(-c7ccccc7)cccc65)c4-c4ccccc4)c3)cc2)cc1]

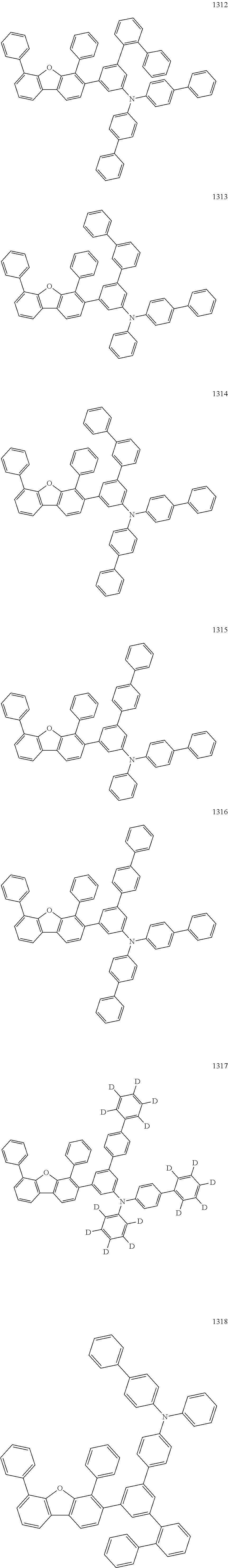

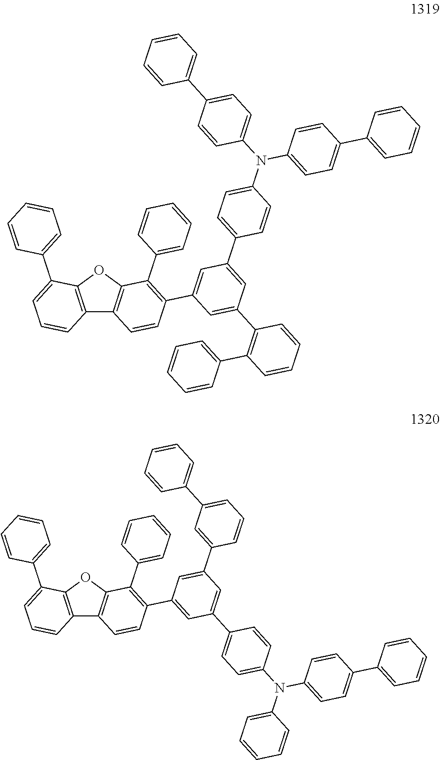 [SMILES: c1ccc(-c2ccc(N(c3ccc(-c4ccccc4)cc3)c3ccc(-c4cc(-c5ccccc5-c5ccccc5)cc(-c5ccc6c(oc7c(-c8ccccc8)cccc76)c5-c5ccccc5)c4)cc3)cc2)cc1 ; c1ccc(-c2ccc(N(c3ccccc3)c3ccc(-c4cc(-c5cccc(-c6ccccc6)c5)cc(-c5ccc6c(oc7c(-c8ccccc8)cccc76)c5-c5ccccc5)c4)cc3)cc2)cc1]

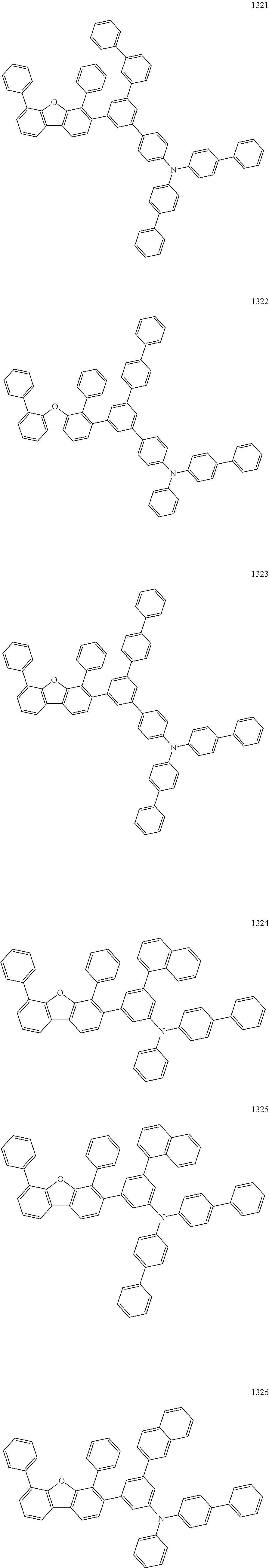

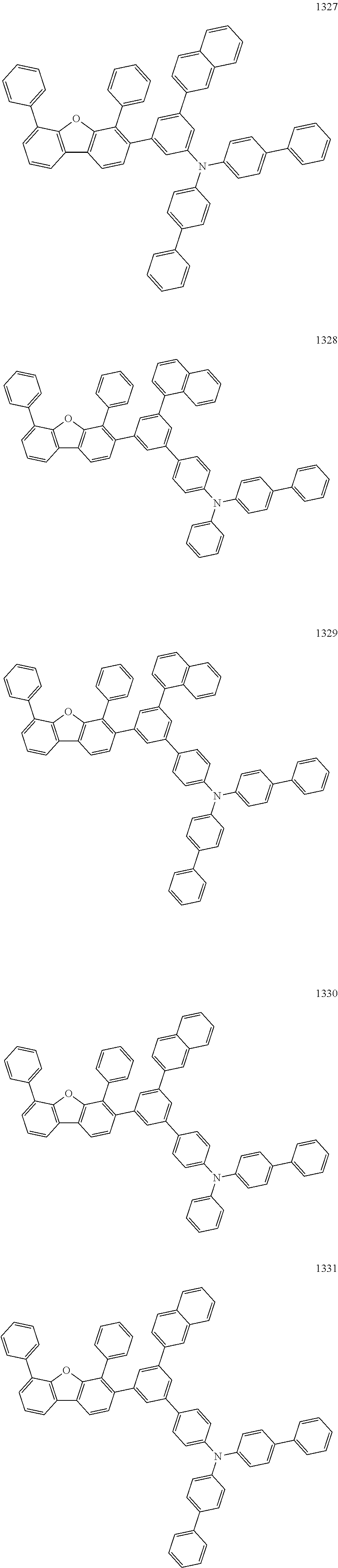

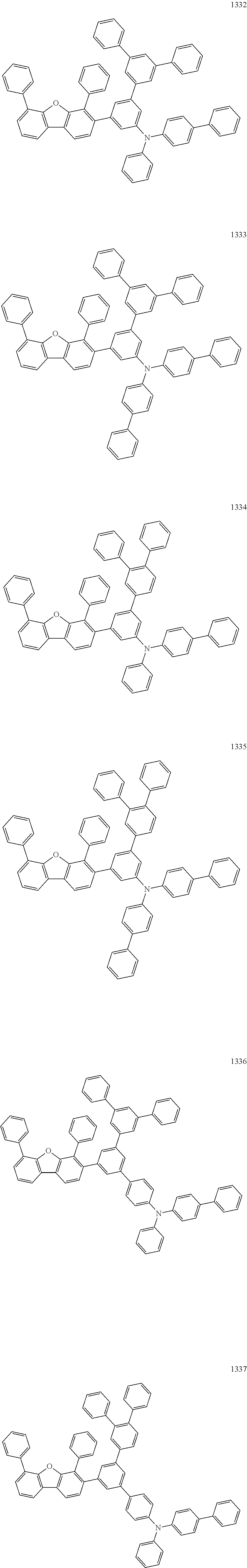

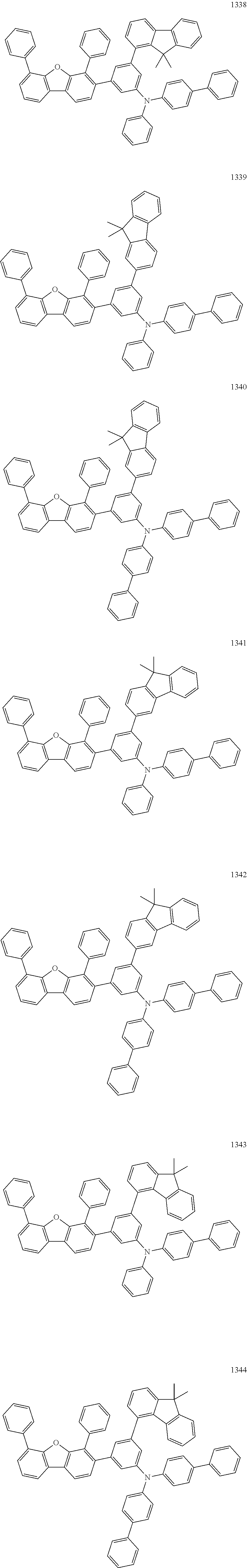

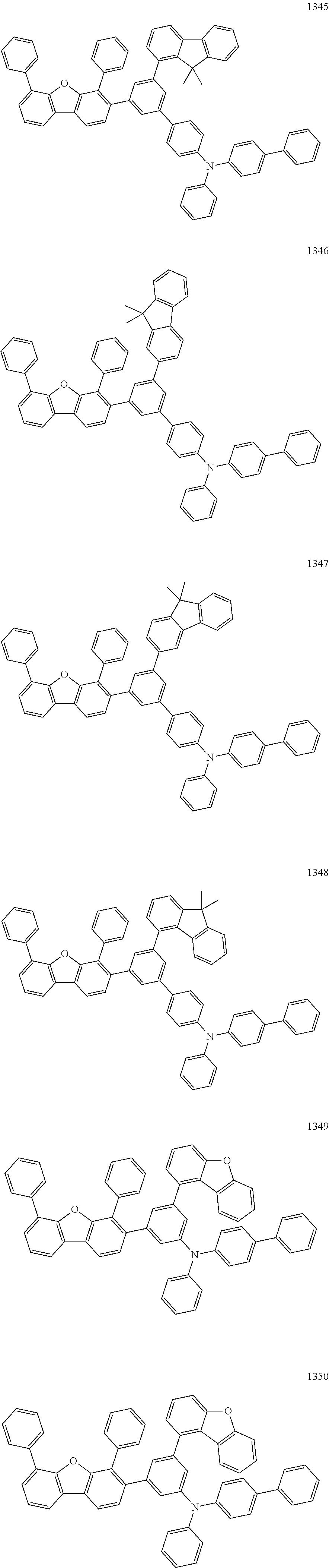

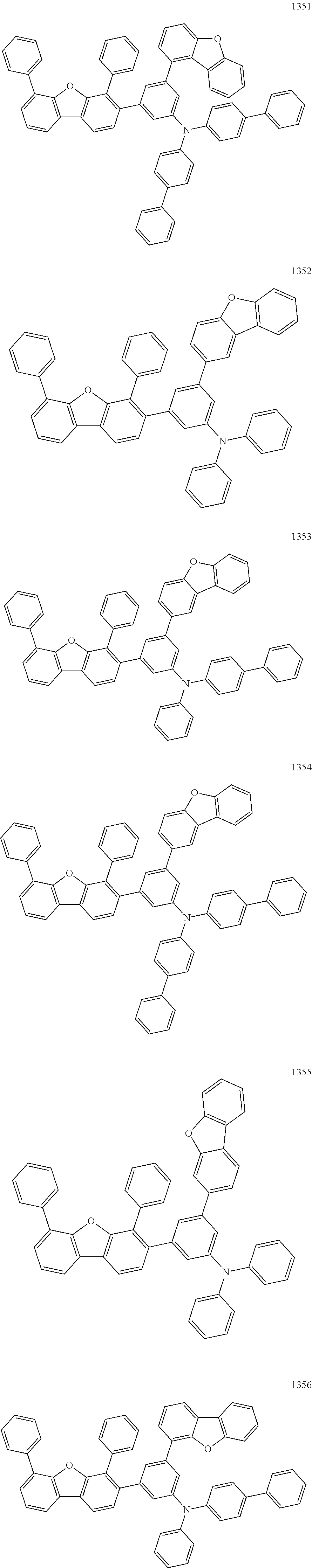

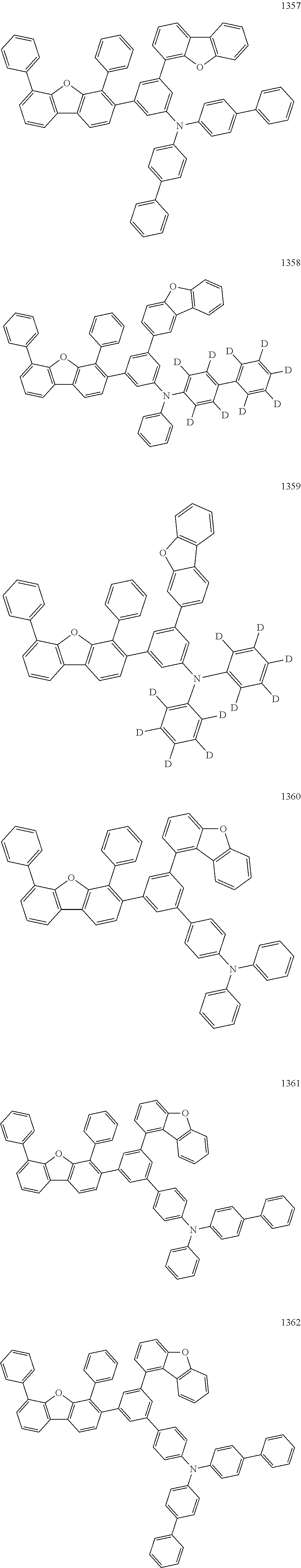

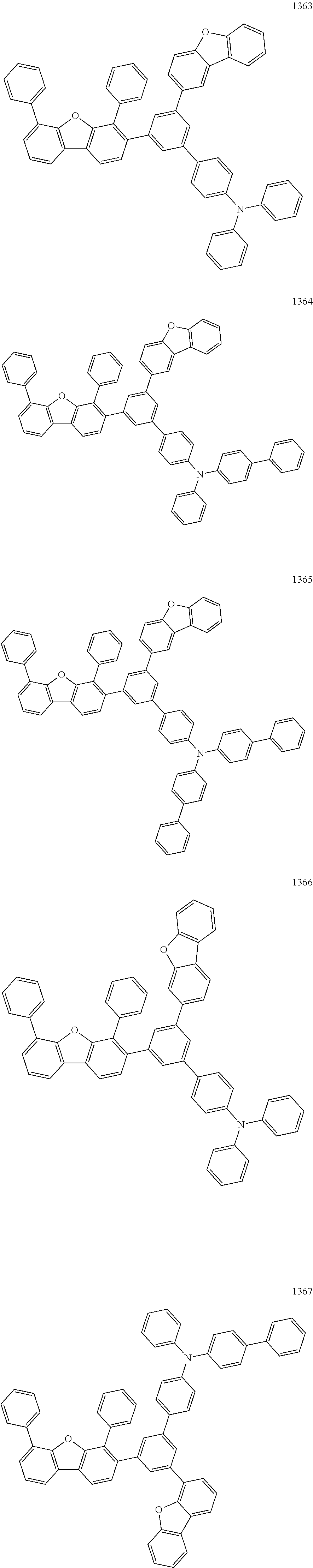

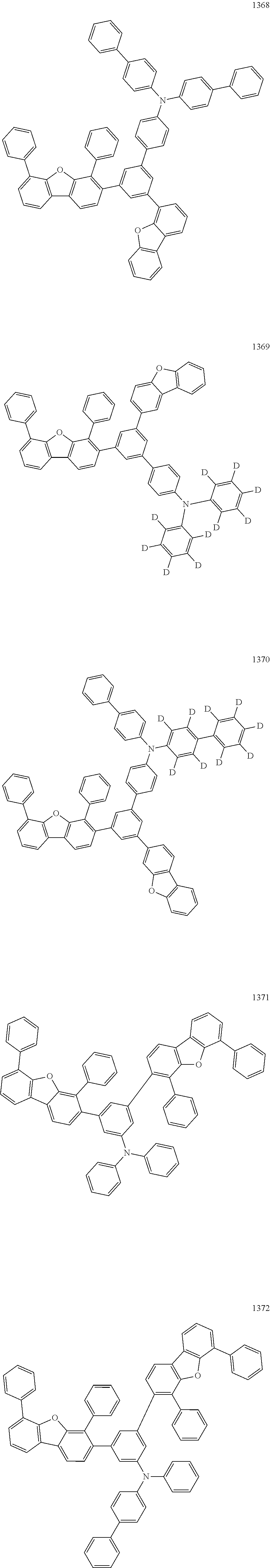

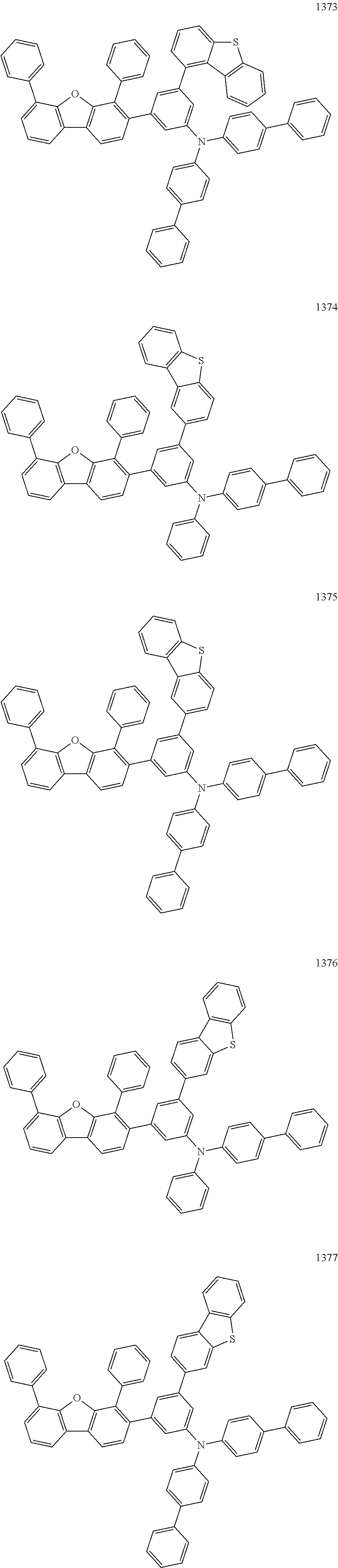

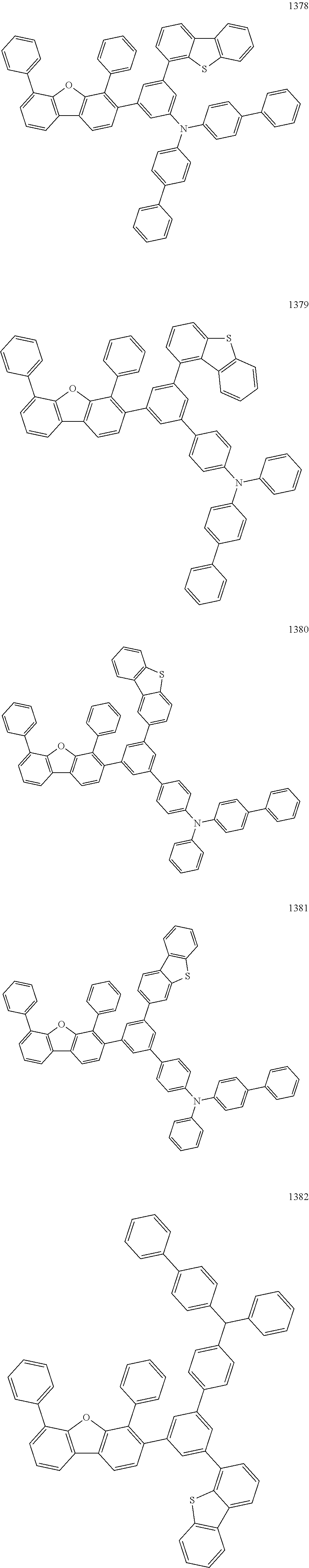

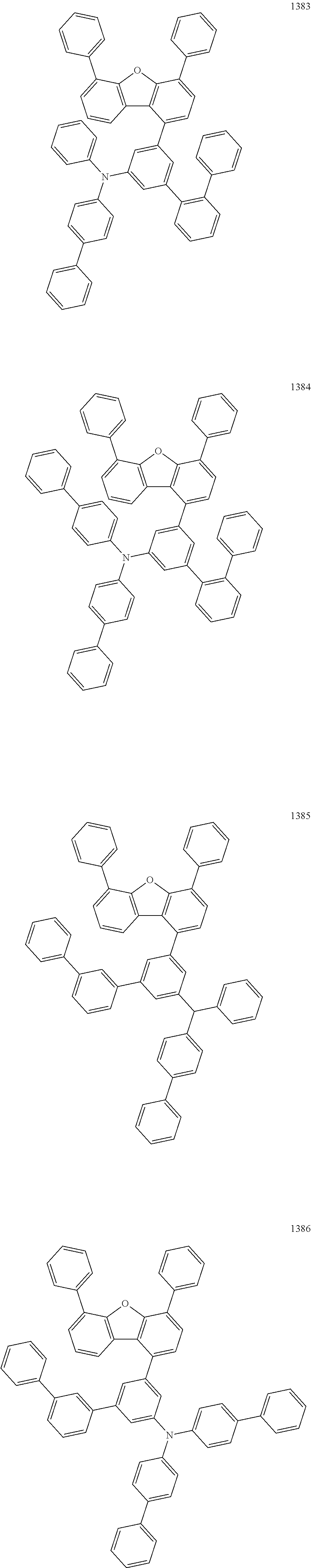 [SMILES: c1ccc(-c2ccc(N(c3ccccc3)c3cc(-c4ccccc4-c4ccccc4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4ccccc4-c4ccccc4)cc(-c4ccc(-c5ccccc5)c5oc6c(-c7ccccc7)cccc6c45)c3)cc2)cc1 ; c1ccc(-c2ccc(C(c3ccccc3)c3cc(-c4cccc(-c5ccccc5)c4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4cccc(-c5ccccc5)c4)cc(-c4ccc(-c5ccccc5)c5oc6c(-c7ccccc7)cccc6c45)c3)cc2)cc1]

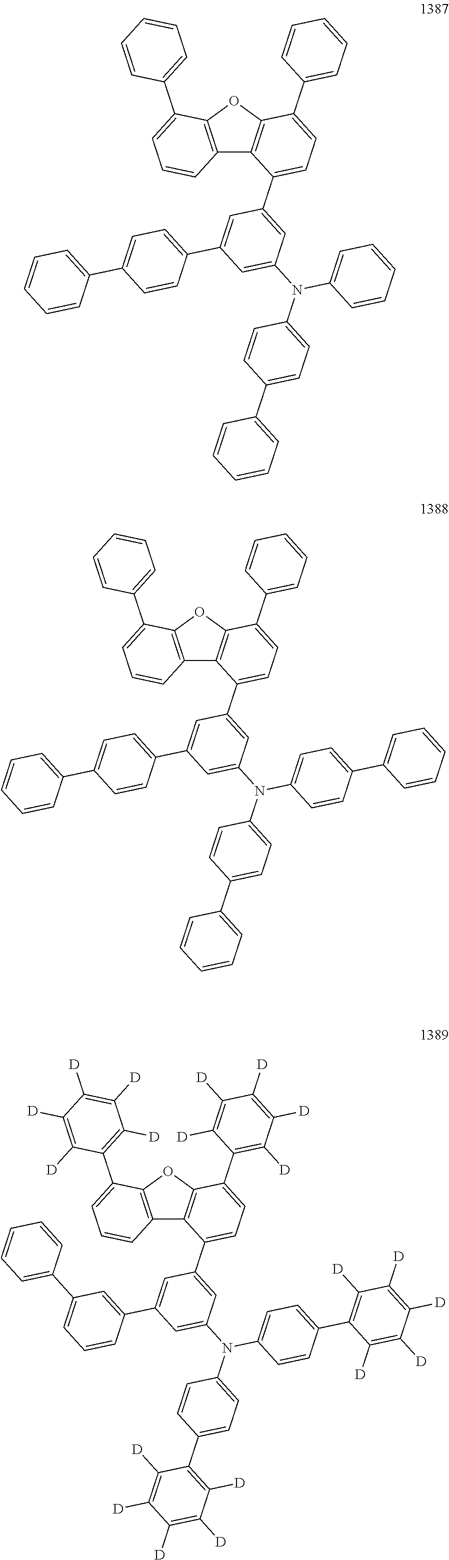 [SMILES: c1ccc(-c2ccc(-c3cc(-c4ccc(-c5ccccc5)c5oc6c(-c7ccccc7)cccc6c45)cc(N(c4ccccc4)c4ccc(-c5ccccc5)cc4)c3)cc2)cc1 ; c1ccc(-c2ccc(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)cc2)cc1 ; [2H]c1c([2H])c([2H])c(-c2ccc(N(c3ccc(-c4c([2H])c([2H])c([2H])c([2H])c4[2H])cc3)c3cc(-c4cccc(-c5ccccc5)c4)cc(-c4ccc(-c5c([2H])c([2H])c([2H])c([2H])c5[2H])c5oc6c(-c7c([2H])c([2H])c([2H])c([2H])c7[2H])cccc6c45)c3)cc2)c([2H])c1[2H]]

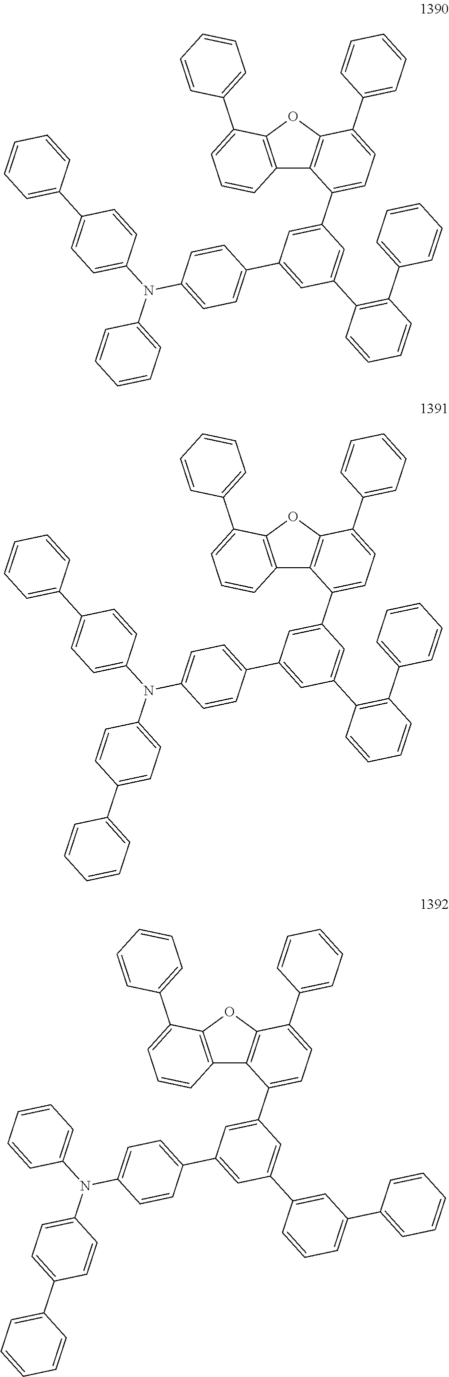 [SMILES: c1ccc(-c2ccc(N(c3ccccc3)c3ccc(-c4cc(-c5ccccc5-c5ccccc5)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5ccccc5-c5ccccc5)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5cccc(-c6ccccc6)c5)cc(-c5ccc(-c6ccccc6)c6oc7c(-c8ccccc8)cccc7c56)c4)cc3)cc2)cc1]

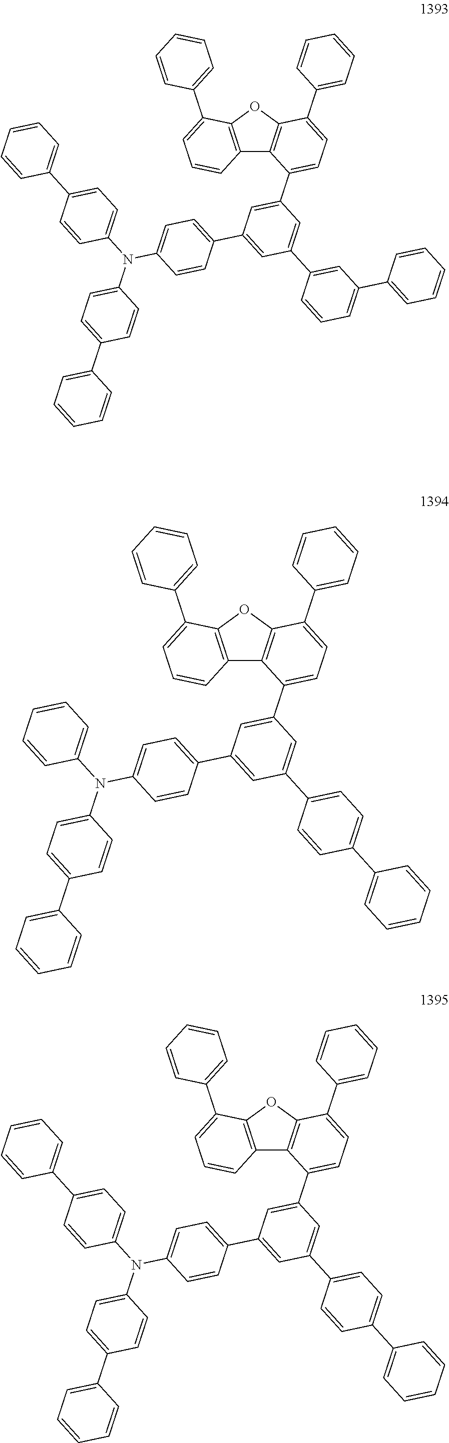 [SMILES: c1ccc(-c2ccc(N(c3ccc(-c4ccccc4)cc3)c3ccc(-c4cc(-c5cccc(-c6ccccc6)c5)cc(-c5ccc(-c6ccccc6)c6oc7c(-c8ccccc8)cccc7c56)c4)cc3)cc2)cc1 ; c1ccc(-c2ccc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)cc2)cc1 ; c1ccc(-c2ccc(-c3cc(-c4ccc(N(c5ccc(-c6ccccc6)cc5)c5ccc(-c6ccccc6)cc5)cc4)cc(-c4ccc(-c5ccccc5)c5oc6c(-c7ccccc7)cccc6c45)c3)cc2)cc1]

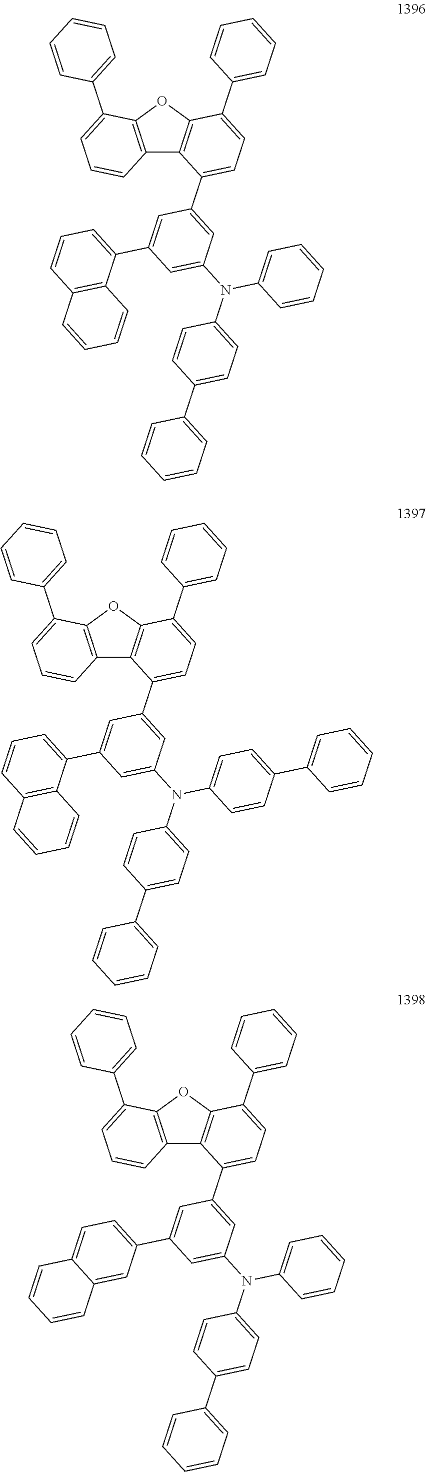

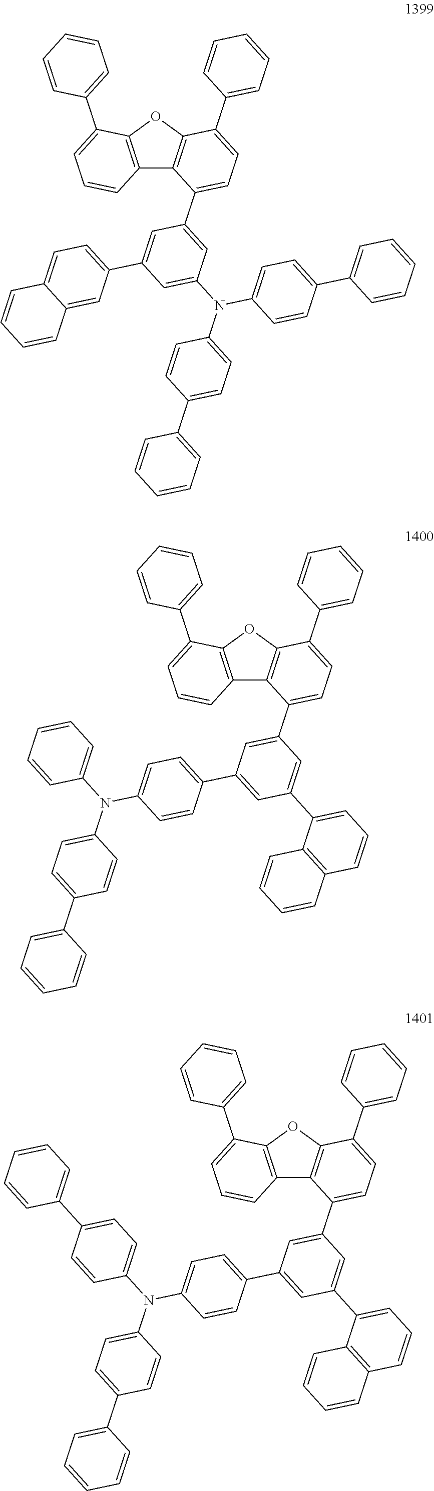 [SMILES: c1ccc(-c2ccc(N(c3ccc(-c4ccccc4)cc3)c3cc(-c4ccc5ccccc5c4)cc(-c4ccc(-c5ccccc5)c5oc6c(-c7ccccc7)cccc6c45)c3)cc2)cc1 ; c1ccc(-c2ccc(N(c3ccccc3)c3ccc(-c4cc(-c5cccc6ccccc56)cc(-c5ccc(-c6ccccc6)c6oc7c(-c8ccccc8)cccc7c56)c4)cc3)cc2)cc1 ; c1ccc(-c2ccc(N(c3ccc(-c4ccccc4)cc3)c3ccc(-c4cc(-c5cccc6ccccc56)cc(-c5ccc(-c6ccccc6)c6oc7c(-c8ccccc8)cccc7c56)c4)cc3)cc2)cc1]

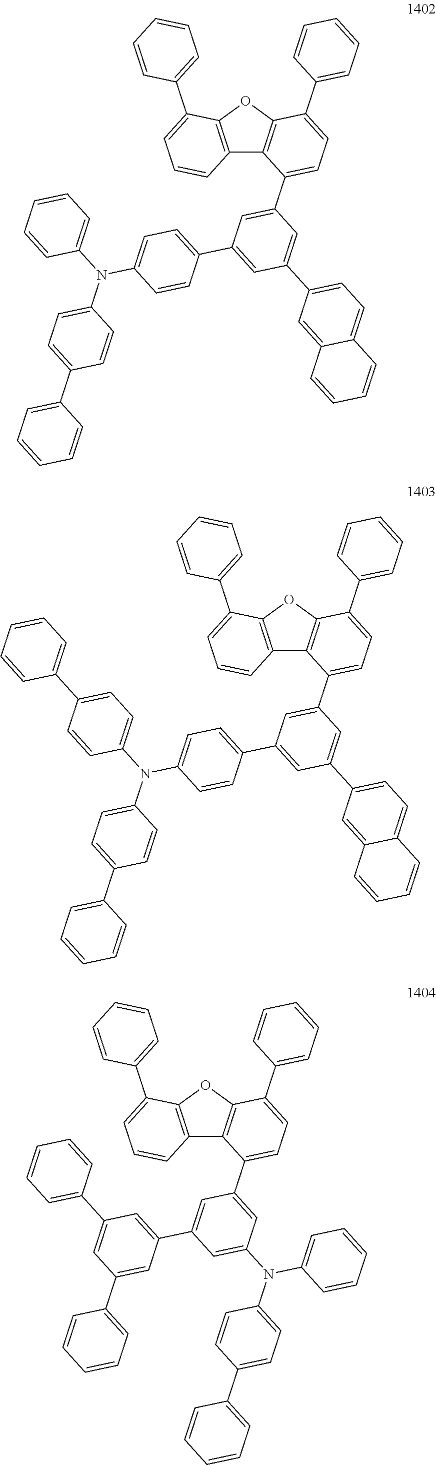

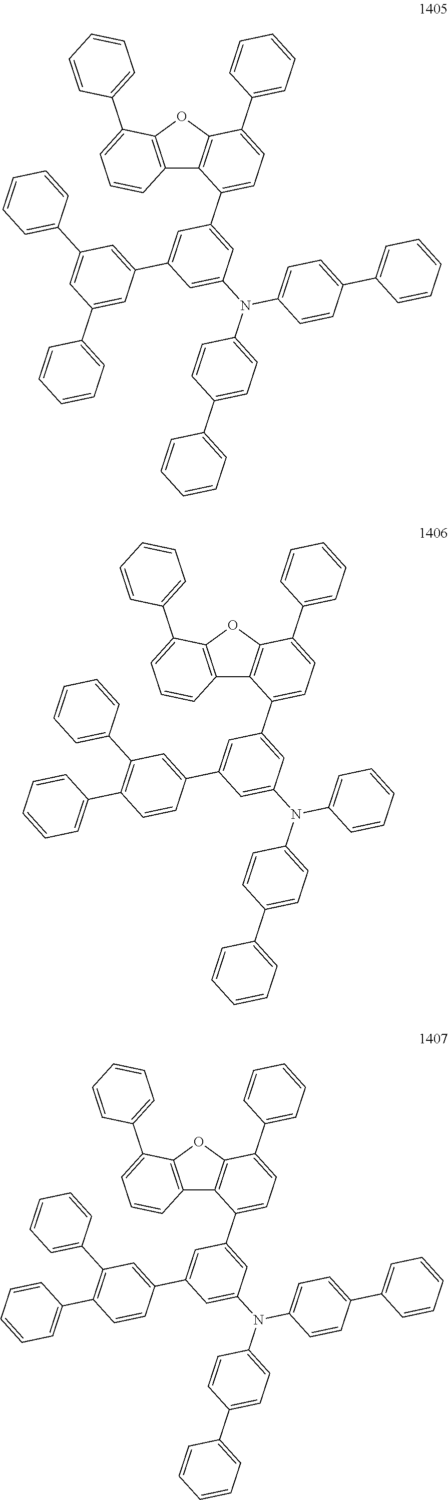 [SMILES: c1ccc(-c2ccc(N(c3ccc(-c4ccccc4)cc3)c3cc(-c4cc(-c5ccccc5)cc(-c5ccccc5)c4)cc(-c4ccc(-c5ccccc5)c5oc6c(-c7ccccc7)cccc6c45)c3)cc2)cc1 ; c1ccc(-c2ccc(N(c3ccccc3)c3cc(-c4ccc(-c5ccccc5)c(-c5ccccc5)c4)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4ccc(-c5ccccc5)c(-c5ccccc5)c4)cc(-c4ccc(-c5ccccc5)c5oc6c(-c7ccccc7)cccc6c45)c3)cc2)cc1]

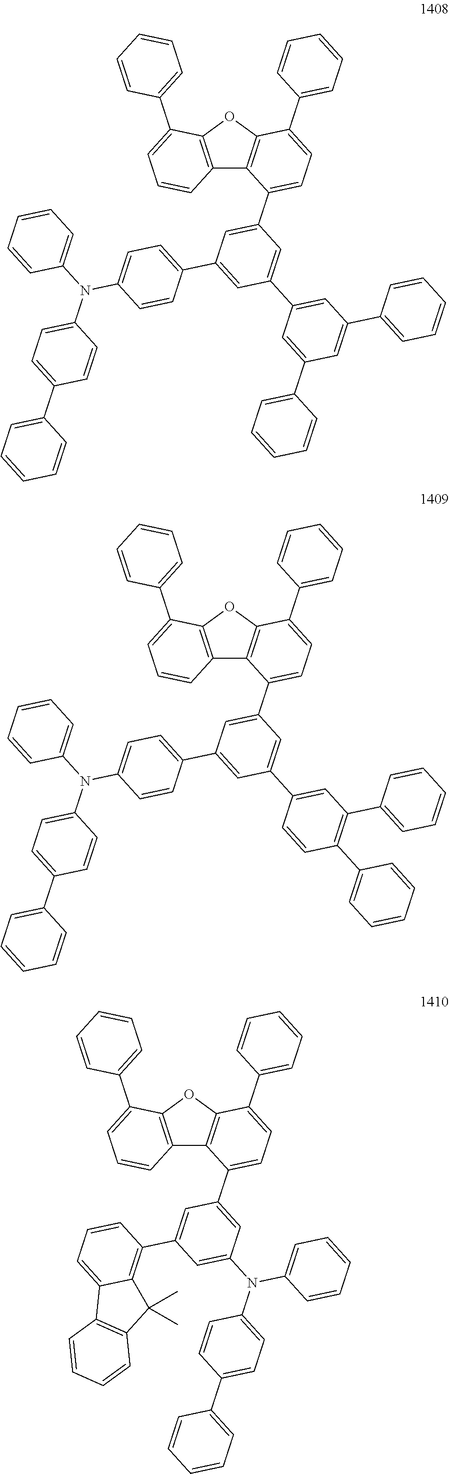 [SMILES: c1ccc(-c2ccc(N(c3ccccc3)c3ccc(-c4cc(-c5cc(-c6ccccc6)cc(-c6ccccc6)c5)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5ccc(-c6ccccc6)c(-c6ccccc6)c5)cc(-c5ccc(-c6ccccc6)c6oc7c(-c8ccccc8)cccc7c56)c4)cc3)cc2)cc1 ; CC1(C)c2ccccc2-c2cccc(-c3cc(-c4ccc(-c5ccccc5)c5oc6c(-c7ccccc7)cccc6c45)cc(N(c4ccccc4)c4ccc(-c5ccccc5)cc4)c3)c21]

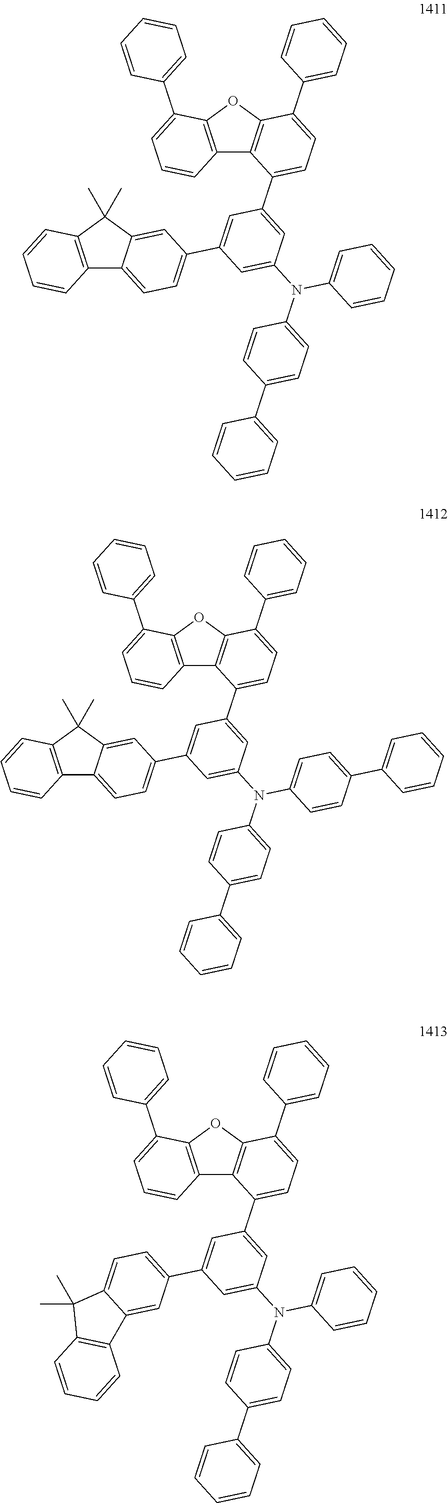 [SMILES: CC1(C)c2ccccc2-c2ccc(-c3cc(-c4ccc(-c5ccccc5)c5oc6c(-c7ccccc7)cccc6c45)cc(N(c4ccccc4)c4ccc(-c5ccccc5)cc4)c3)cc21 ; CC1(C)c2ccccc2-c2ccc(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)cc21 ; CC1(C)c2ccccc2-c2cc(-c3cc(-c4ccc(-c5ccccc5)c5oc6c(-c7ccccc7)cccc6c45)cc(N(c4ccccc4)c4ccc(-c5ccccc5)cc4)c3)ccc21]

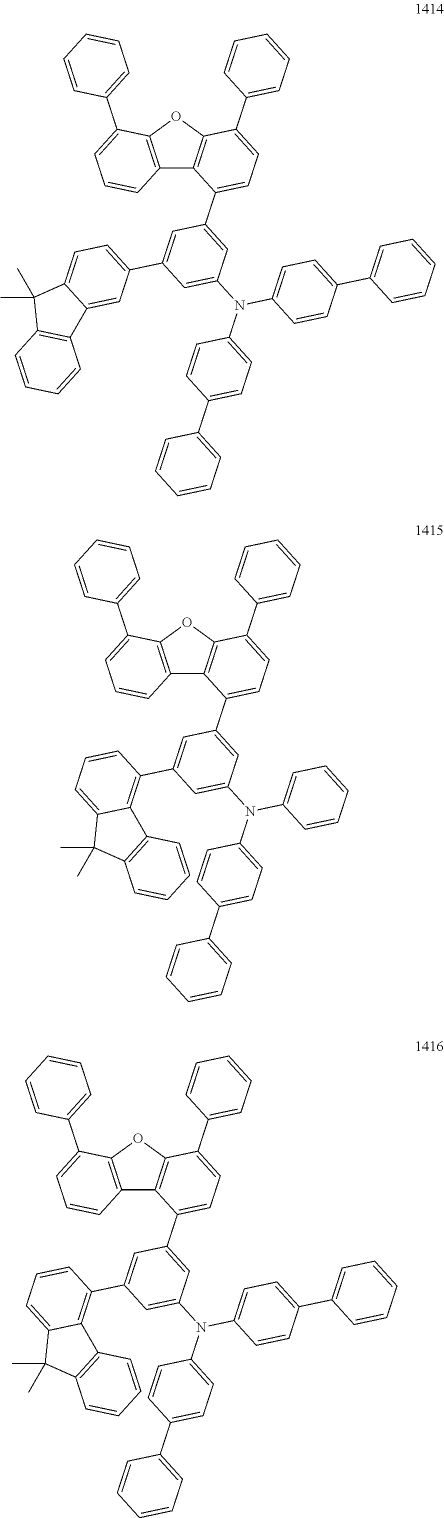 [SMILES: CC1(C)c2ccccc2-c2cc(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)ccc21 ; CC1(C)c2ccccc2-c2c(-c3cc(-c4ccc(-c5ccccc5)c5oc6c(-c7ccccc7)cccc6c45)cc(N(c4ccccc4)c4ccc(-c5ccccc5)cc4)c3)cccc21 ; CC1(C)c2ccccc2-c2c(-c3cc(-c4ccc(-c5ccccc5)c5oc6c(-c7ccccc7)cccc6c45)cc(N(c4ccc(-c5ccccc5)cc4)c4ccc(-c5ccccc5)cc4)c3)cccc21]

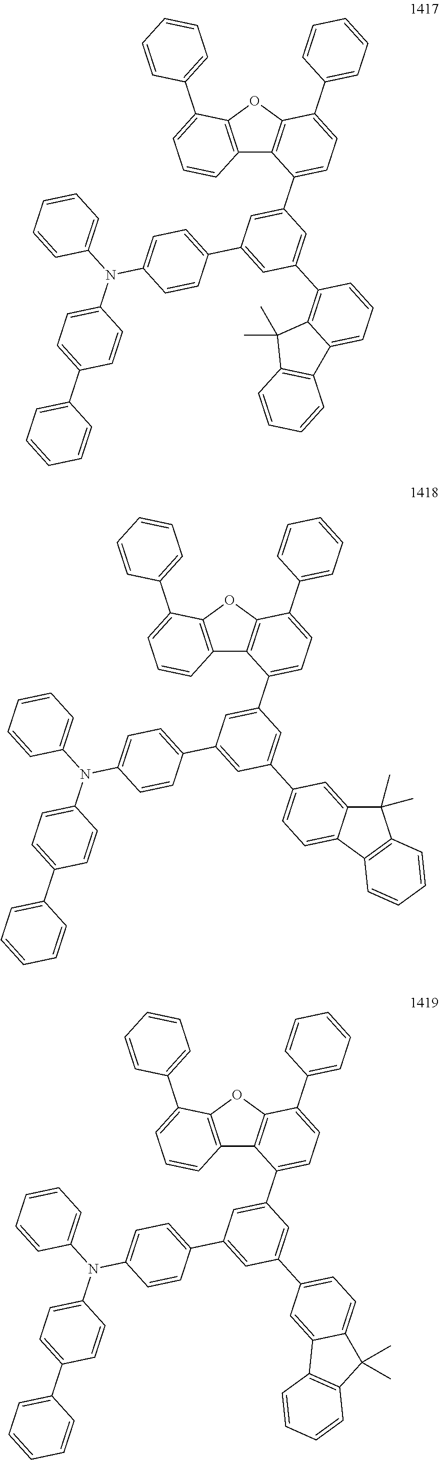 [SMILES: CC1(C)c2ccccc2-c2cccc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)c21 ; CC1(C)c2ccccc2-c2ccc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)cc21 ; CC1(C)c2ccccc2-c2cc(-c3cc(-c4ccc(N(c5ccccc5)c5ccc(-c6ccccc6)cc5)cc4)cc(-c4ccc(-c5ccccc5)c5oc6c(-c7ccccc7)cccc6c45)c3)ccc21]

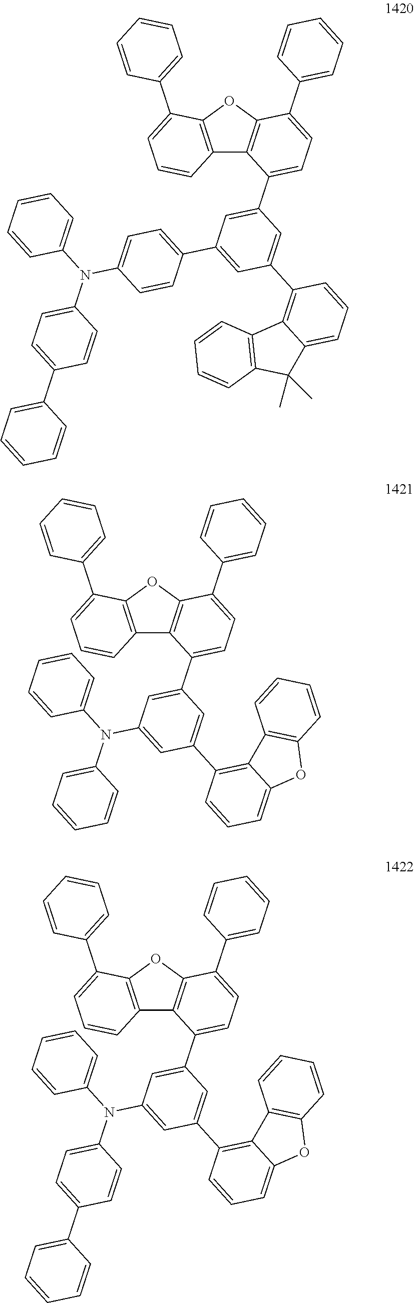

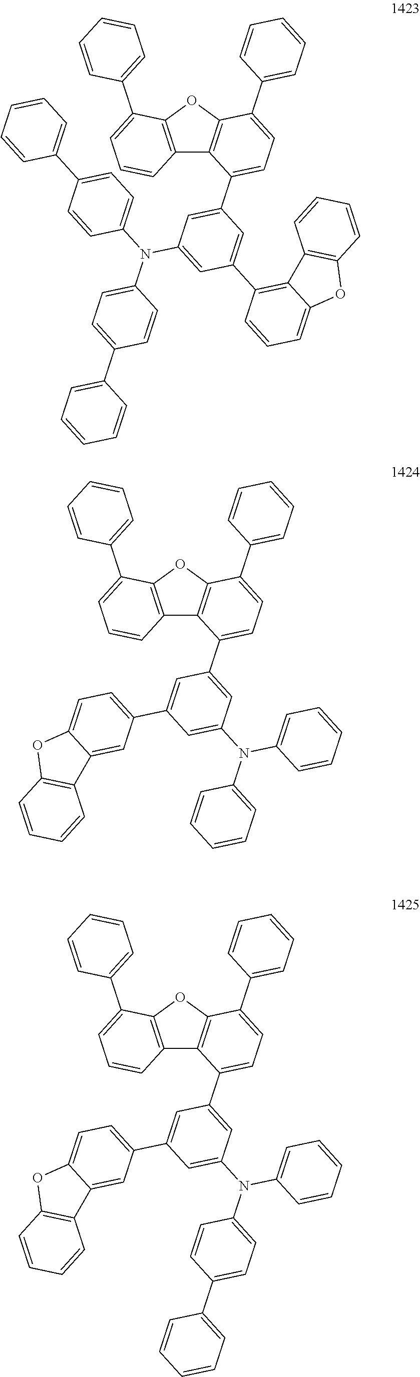

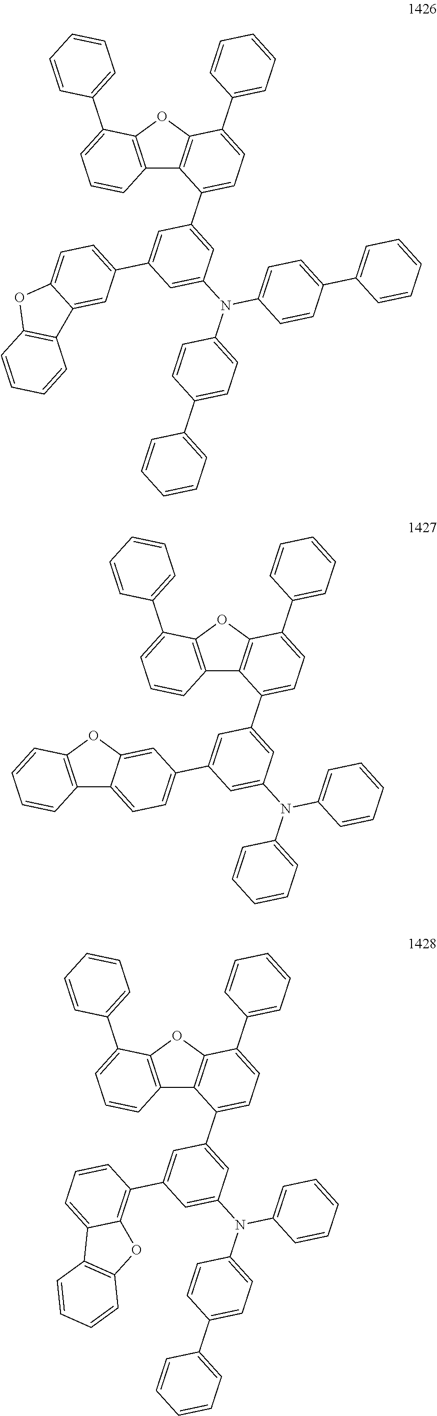 [SMILES: c1ccc(-c2ccc(N(c3ccc(-c4ccccc4)cc3)c3cc(-c4ccc5oc6ccccc6c5c4)cc(-c4ccc(-c5ccccc5)c5oc6c(-c7ccccc7)cccc6c45)c3)cc2)cc1 ; c1ccc(-c2cccc3c2oc2c(-c4ccccc4)ccc(-c4cc(-c5ccc6c(c5)oc5ccccc56)cc(N(c5ccccc5)c5ccccc5)c4)c23)cc1 ; c1ccc(-c2ccc(N(c3ccccc3)c3cc(-c4cccc5c4oc4ccccc45)cc(-c4ccc(-c5ccccc5)c5oc6c(-c7ccccc7)cccc6c45)c3)cc2)cc1]

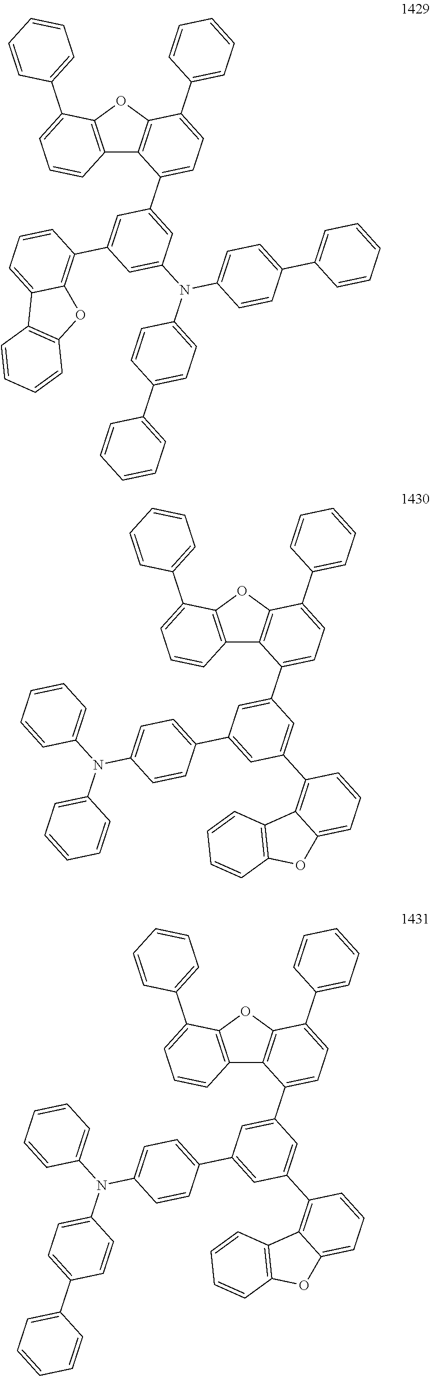

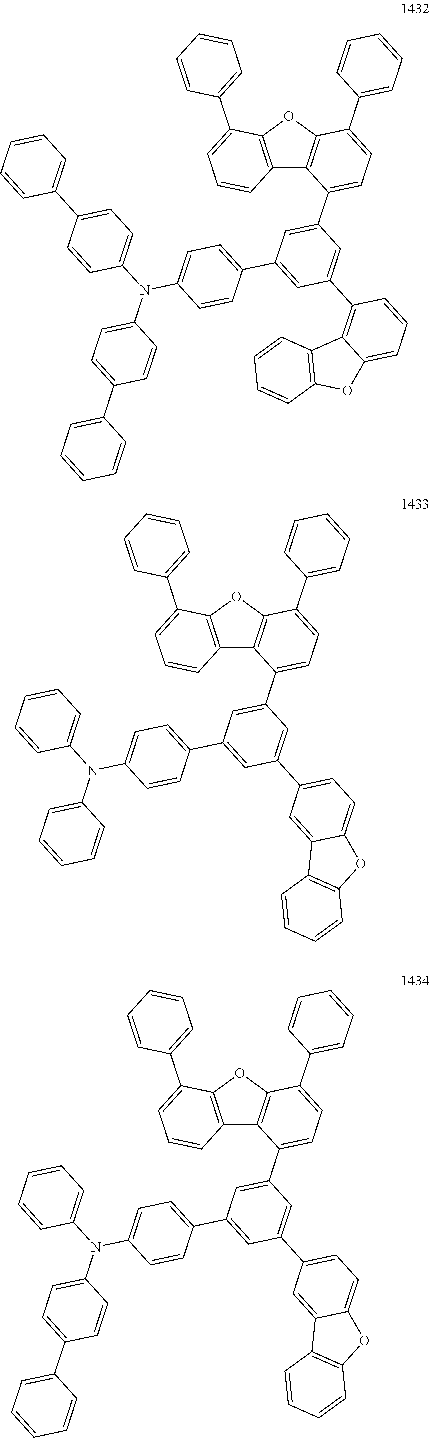 [SMILES: c1ccc(-c2ccc(N(c3ccc(-c4ccccc4)cc3)c3ccc(-c4cc(-c5cccc6oc7ccccc7c56)cc(-c5ccc(-c6ccccc6)c6oc7c(-c8ccccc8)cccc7c56)c4)cc3)cc2)cc1 ; c1ccc(-c2cccc3c2oc2c(-c4ccccc4)ccc(-c4cc(-c5ccc(N(c6ccccc6)c6ccccc6)cc5)cc(-c5ccc6oc7ccccc7c6c5)c4)c23)cc1 ; c1ccc(-c2ccc(N(c3ccccc3)c3ccc(-c4cc(-c5ccc6oc7ccccc7c6c5)cc(-c5ccc(-c6ccccc6)c6oc7c(-c8ccccc8)cccc7c56)c4)cc3)cc2)cc1]

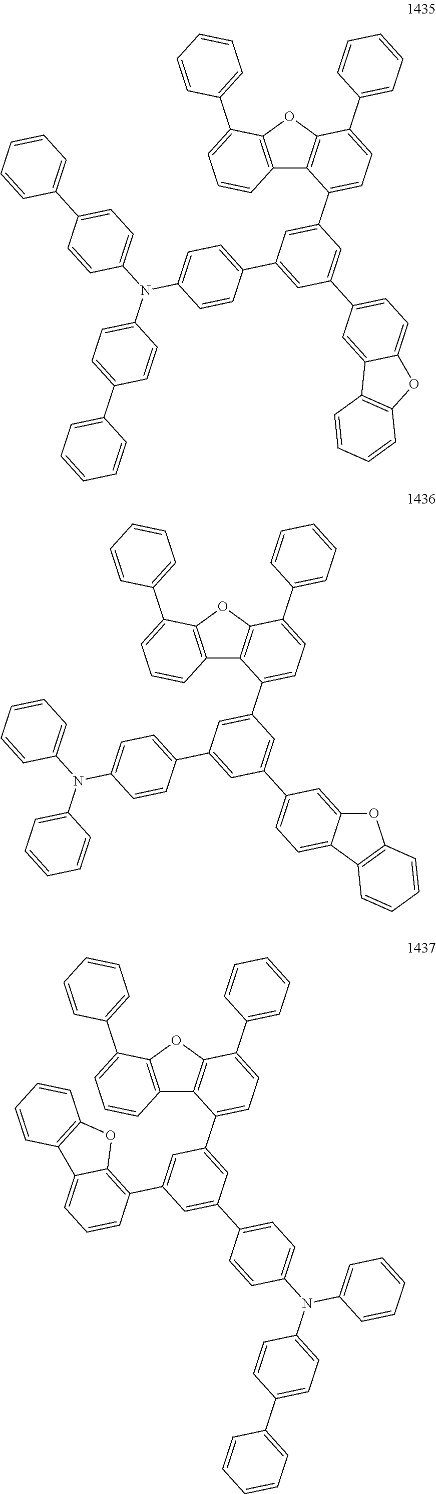 [SMILES: c1ccc(-c2ccc(N(c3ccc(-c4ccccc4)cc3)c3ccc(-c4cc(-c5ccc6oc7ccccc7c6c5)cc(-c5ccc(-c6ccccc6)c6oc7c(-c8ccccc8)cccc7c56)c4)cc3)cc2)cc1 ; c1ccc(-c2cccc3c2oc2c(-c4ccccc4)ccc(-c4cc(-c5ccc(N(c6ccccc6)c6ccccc6)cc5)cc(-c5ccc6c(c5)oc5ccccc56)c4)c23)cc1 ; c1ccc(-c2ccc(N(c3ccccc3)c3ccc(-c4cc(-c5cccc6c5oc5ccccc56)cc(-c5ccc(-c6ccccc6)c6oc7c(-c8ccccc8)cccc7c56)c4)cc3)cc2)cc1]

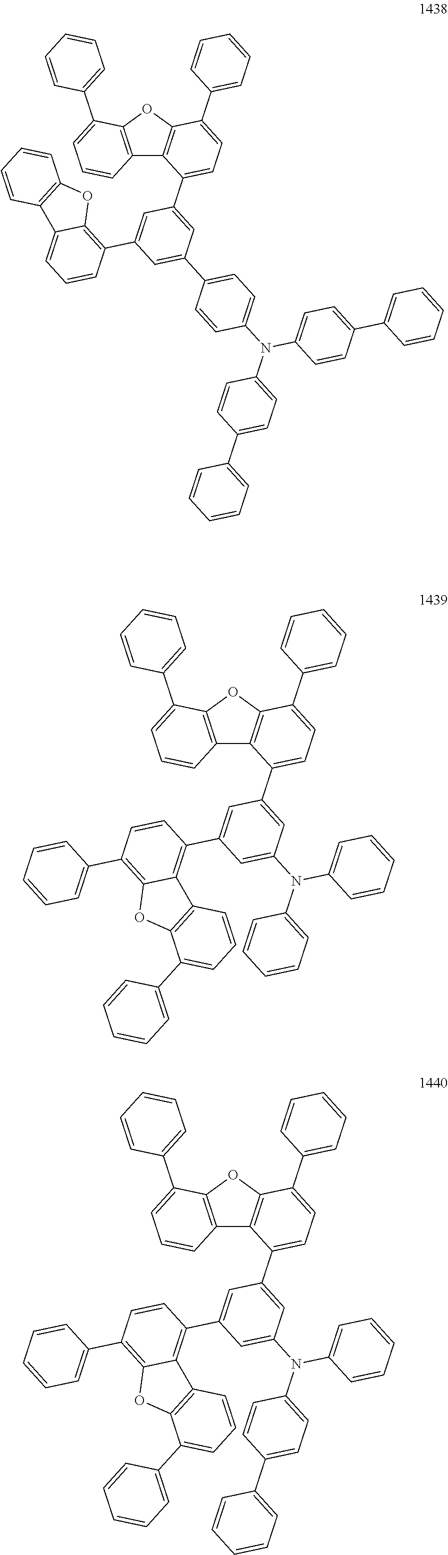 [SMILES: c1ccc(-c2ccc(N(c3ccc(-c4ccccc4)cc3)c3ccc(-c4cc(-c5cccc6c5oc5ccccc56)cc(-c5ccc(-c6ccccc6)c6oc7c(-c8ccccc8)cccc7c56)c4)cc3)cc2)cc1 ; c1ccc(-c2cccc3c2oc2c(-c4ccccc4)ccc(-c4cc(-c5ccc(-c6ccccc6)c6oc7c(-c8ccccc8)cccc7c56)cc(N(c5ccccc5)c5ccccc5)c4)c23)cc1 ; c1ccc(-c2ccc(N(c3ccccc3)c3cc(-c4ccc(-c5ccccc5)c5oc6c(-c7ccccc7)cccc6c45)cc(-c4ccc(-c5ccccc5)c5oc6c(-c7ccccc7)cccc6c45)c3)cc2)cc1]

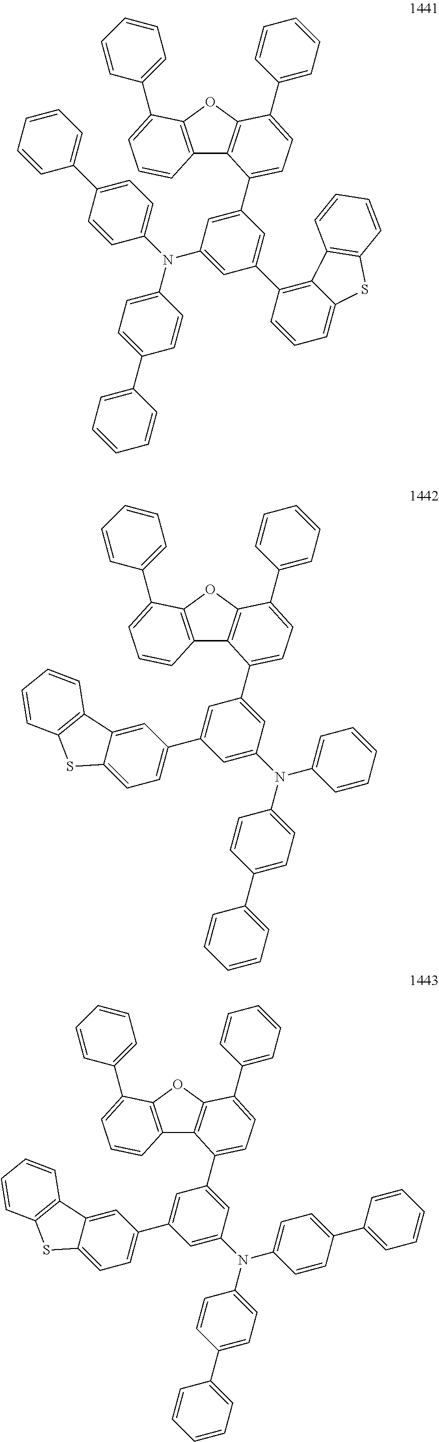

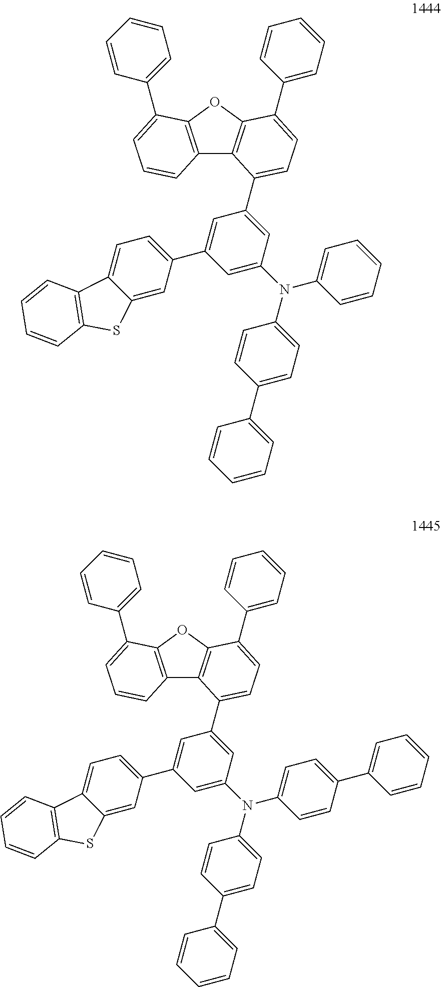 [SMILES: c1ccc(-c2ccc(N(c3ccccc3)c3cc(-c4ccc5c(c4)sc4ccccc45)cc(-c4ccc(-c5ccccc5)c5oc6c(-c7ccccc7)cccc6c45)c3)cc2)cc1 ; c1ccc(-c2ccc(N(c3ccc(-c4ccccc4)cc3)c3cc(-c4ccc5c(c4)sc4ccccc45)cc(-c4ccc(-c5ccccc5)c5oc6c(-c7ccccc7)cccc6c45)c3)cc2)cc1]

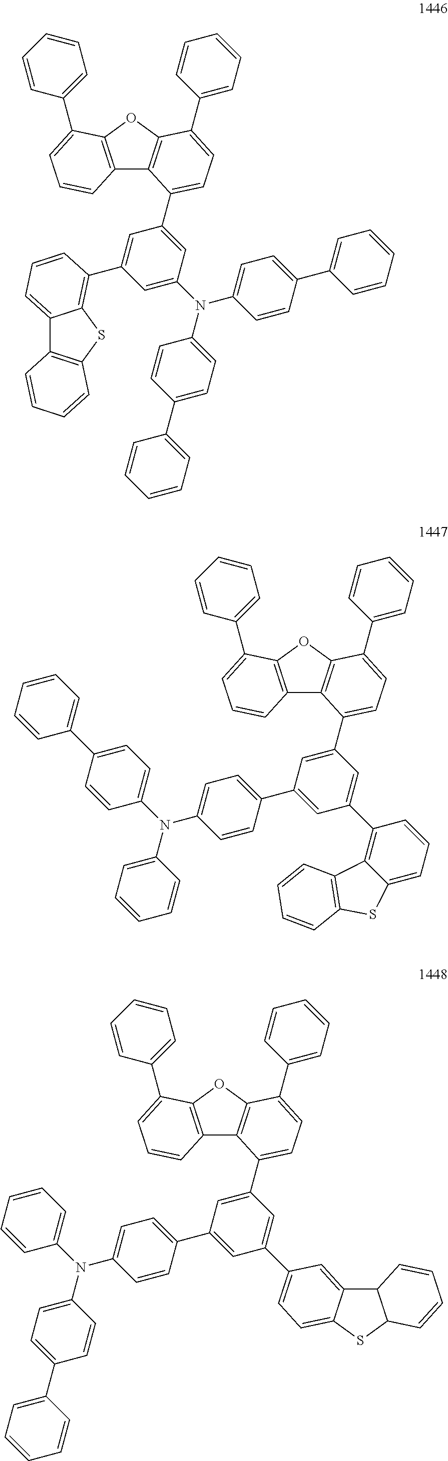 [SMILES: c1ccc(-c2ccc(N(c3ccc(-c4ccccc4)cc3)c3cc(-c4cccc5c4sc4ccccc45)cc(-c4ccc(-c5ccccc5)c5oc6c(-c7ccccc7)cccc6c45)c3)cc2)cc1 ; c1ccc(-c2ccc(N(c3ccccc3)c3ccc(-c4cc(-c5cccc6sc7ccccc7c56)cc(-c5ccc(-c6ccccc6)c6oc7c(-c8ccccc8)cccc7c56)c4)cc3)cc2)cc1 ; C1=CC2Sc3ccc(-c4cc(-c5ccc(N(c6ccccc6)c6ccc(-c7ccccc7)cc6)cc5)cc(-c5ccc(-c6ccccc6)c6oc7c(-c8ccccc8)cccc7c56)c4)cc3C2C=C1]

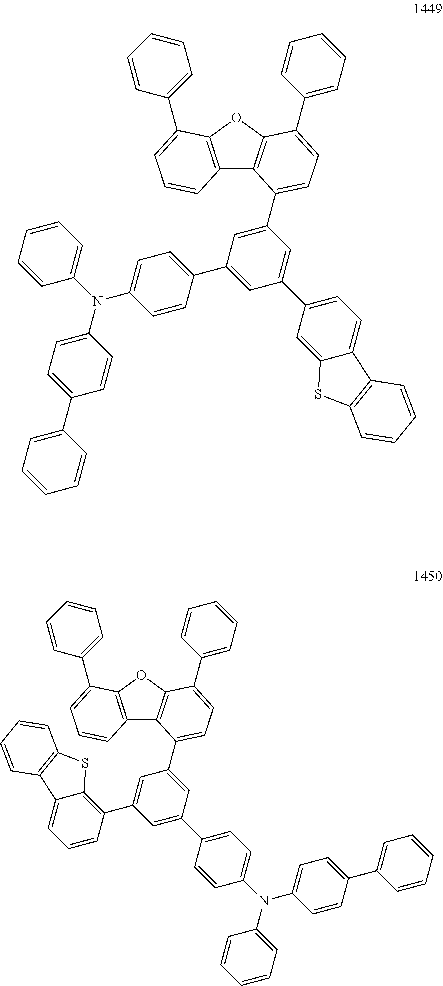 [SMILES: c1ccc(-c2ccc(N(c3ccccc3)c3ccc(-c4cc(-c5ccc6c(c5)sc5ccccc56)cc(-c5ccc(-c6ccccc6)c6oc7c(-c8ccccc8)cccc7c56)c4)cc3)cc2)cc1 ; c1ccc(-c2ccc(N(c3ccccc3)c3ccc(-c4cc(-c5cccc6c5sc5ccccc56)cc(-c5ccc(-c6ccccc6)c6oc7c(-c8ccccc8)cccc7c56)c4)cc3)cc2)cc1]

8. An organic light-emitting device comprising a first electrode, a second electrode, and an organic layer having one or more layers disposed between the first electrode and the second electrode, wherein one or more layers of the organic layer comprise one or more of the organic compounds implemented by [Formula I] according to claim 1.

9. The organic light-emitting device of claim 8, wherein the organic layer comprises one or more layers of an electron injecting layer, an electron transport layer, a hole injecting layer, a hole transport layer, an electron blocking layer, a hole blocking layer, and a light-emitting layer, and
one or more layers of the layers comprise the organic compound represented by [Formula I] above.

10. The organic light-emitting device of claim 9, wherein the electron blocking layer comprises the organic compound represented by [Formula I] above.
